(19) United States
(12) Patent Application Publication
(10) Pub. No.: US 2023/0212606 A1
(43) Pub. Date: Jul. 6, 2023

(54) COMPOSITIONS AND METHODS FOR TREATING KCNQ4-ASSOCIATED HEARING LOSS

(71) Applicant: Akouos, Inc., Boston, MA (US)

(72) Inventors: Emmanuel John Simons, Brookline, MA (US); Robert Ng, Newton, MA (US); Linda B. Couto, Pleasanton, CA (US); Gregory Scott Robinson, Wilmington, MA (US); Katherine Diane Gribble, Chestnut Hill, MA (US)

(21) Appl. No.: 17/923,636

(22) PCT Filed: May 12, 2021
(86) PCT No.: PCT/US21/31939

§ 371 Date: Nov. 7, 2022

Related U.S. Application Data
(60) Provisional application No. 63/024,488, filed on May 13, 2020.

(51) Int. Cl. C12N 15/86; C07K 14/47

(52) U.S. Cl. CPC C12N 15/86; C07K 14/47; C12N 2750/14143

(57) ABSTRACT

The present disclosure provides technologies comprising a polynucleotide capable of expressing and/or inhibiting a KCNQ4 gene product.

### CROSS-REFERENCE TO RELATED APPLICATION

The application claims the benefit of U.S. Application Ser. No. 63/024,488 filed on May 13, 2020, the disclosure of which is hereby incorporated by reference in its entirety.

### SEQUENCE LISTING

The instant application contains a Sequence Listing which has been submitted electronically in ASCII format and is hereby incorporated by reference in its entirety. Said ASCII copy, created on May 11, 2021, is named 2013615-0286_SL.txt and is 819,483 bytes in size.

### BACKGROUND

There are many types of hearing loss. Some hearing loss is related to one or more genes. The present disclosure provides technologies related to KCNQ4-associated hearing loss.

### SUMMARY

The present disclosure recognizes that diseases or conditions associated with hearing loss can be treated via, e.g., replacement, addition and/or inhibition of certain gene products. The present disclosure further provides that gene products involved in development, function, and/or maintenance of ear cells, e.g., inner ear cells, e.g., hair cells can be useful for treatment of diseases or conditions associated with cell loss, e.g., hair cell loss. Accordingly, the present disclosure provides various technologies including those for methods of making, using, and/or administering compositions to express a gene product involved in the development, function, and/or maintenance of inner ear cells, e.g., hair cells.

Technologies provided by the present disclosure also include use of such compositions in treatment of hearing loss, or diseases or conditions associated with hearing loss. In some embodiments, a gene product can be encoded by a KCNQ4 gene or a characteristic portion thereof. In some embodiments, a gene product can be KCNQ4 protein or a characteristic portion thereof. In some embodiments, a variant KCNQ4 gene product is inhibited. In some embodiments, the present disclosure provides technologies to express functional KCNQ4. In some embodiments, the present disclosure provides technologies to inhibit a KCNQ4 variant. In some embodiments, the present disclosure provides technologies to both express functional KCNQ4 and inhibit a KCNQ4 variant.

The present disclosure further provides that viral delivery, e.g., via AAV particles, can be useful for administration of compositions to express gene products involved in development, function, and/or maintenance of inner ear cells, and/or treatment of hearing loss, or diseases or conditions associated with hearing loss. As described herein, AAV particles comprise (i) a AAV polynucleotide construct (e.g., a recombinant AAV polynucleotide construct), and (ii) a capsid comprising capsid proteins. In some embodiments, an AAV polynucleotide construct comprises KCNQ4 gene or a characteristic portion thereof. In some embodiments, AAV particles described herein are referred to as rAAV-KCNQ4 or rAAV-KCNQ4 particles. In some embodiments, AAV particles described herein comprise Anc80 AAV capsid proteins and are referred to as rAAV Anc80-KCNQ4 or rAAV Anc80-KCNQ4 particles.

Among other things, the present disclosure provides a construct comprising a coding sequence operably linked to a promoter, wherein the coding sequence encodes a Kv7.4 protein.

The present disclosure also provides a construct comprising a coding sequence operably linked to a promoter, wherein the coding sequence encodes a KCNQ4 inhibitory nucleic acid, which comprises a nucleotide sequence complementary to a KCNQ4 gene. In some embodiments, AAV particles described herein are referred to as rAAV-KCNQ4-Inhibitory-RNA or rAAV-KCNQ4-Inhibitory-RNA particles. In some embodiments, AAV particles described herein comprise Anc80 AAV capsid proteins and are referred to as rAAV Anc80-KCNQ4-Inhibitory-RNA or rAAV Anc80-KCNQ4-Inhibitory-RNA particles.

In some embodiments, the coding sequence is a KCNQ4 gene. In some embodiments, the KCNQ4 gene is a primate KCNQ4 gene. In some embodiments, the KCNQ4 gene is a human KCNQ4 gene. In some embodiments, the KCNQ4 gene is a murine (or mouse) KCNQ4 gene.

In some embodiments, the human KCNQ4 gene comprises a nucleic acid sequence according to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 25, SEQ ID NO: 26, SEQ ID NO: 27, SEQ ID NO: 28, SEQ ID NO: 29, SEQ ID NO: 30, or SEQ ID NO: 90. In some embodiments, the human KCNQ4 gene comprises a nucleic acid sequence according to SEQ ID NO: 9 or 10. In some embodiments, the mouse (or murine) KCNQ4 gene comprises a nucleic acid sequence according to SEQ ID NO: 91.

In some embodiments, the Kv7.4 protein is a primate Kv7.4 protein. In some embodiments, the Kv7.4 protein is a human Kv7.4 protein. In some embodiments, the Kv7.4 protein is a mouse Kv7.4 protein. In some embodiments, the Kv7.4 protein comprises an amino acid sequence according to SEQ ID NO: 11, SEQ ID NO: 12, SEQ ID NO: 13, or SEQ ID NO: 92.

In some embodiments, the promoter is an inducible promoter, a constitutive promoter, or a tissue-specific promoter. In some embodiments, the promotor is a cochlear hair cell-specific promoter. In some embodiments, the cochlear hair cell-specific promoter is a ATOH1 promoter, a POU4F3 promoter, a LHX3 promoter, a MYO7A promoter, a MYO6 promoter, a α9ACHR promoter, or a α10ACHR promoter. In some embodiments, the promoter is a CAG promoter, a CBA promoter, an smCBA promoter, a CMV promoter, or a CB7 promoter. In some embodiments, the promoter comprises a nucleic acid sequence according to SEQ ID NO: 22, SEQ ID NO: 23, SEQ ID NO: 24, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308, SEQ ID NO: 309, and/or SEQ ID NO: 310.

In some embodiments, a construct of the present disclosure comprises two AAV inverted terminal repeats (ITRs), wherein the two AAV ITRs flank the coding sequence and promoter. In some embodiments, the two AAV ITRs are or are derived from AAV2 ITRs. In some embodiments, the two AAV ITRs comprise: (i) a 5′ ITR comprising a nucleic acid sequence according to SEQ ID NO: 15 and a 3′ ITR comprising a nucleic acid sequence according to SEQ ID NO: 16. In some embodiments, the two AAV ITRs comprise: (i) a 5′ ITR comprising a nucleic acid sequence according to SEQ ID NO: 19 and a 3′ ITR comprising a nucleic acid sequence according to SEQ ID NO: 20. In some embodiments, the construct comprises a nucleic acid sequence according to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 25, SEQ ID NO: 26, SEQ ID NO: 27, SEQ ID NO: 28, SEQ ID NO: 29, SEQ ID NO: 30 or SEQ ID NO: 90. In some embodiments, the construct comprises a nucleic acid sequence according to one or more of SEQ ID NOs: 1-41 and/or 42-70 and/or 96-97.

The present disclosure also provides a construct comprising a coding sequence operably linked to a promoter, wherein the coding sequence encodes a KCNQ4 inhibitory nucleic acid, which comprises a nucleotide sequence complementary to a KCNQ4 gene. In some embodiments, the KCNQ4 inhibitory nucleic acid is an miRNA, an siRNA, or shRNA. In some embodiments, the KCNQ4 inhibitory RNA comprises a sequence according to SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO:63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 96, or SEQ ID NO: 97. In some embodiments, the KCNQ4 inhibitory RNA is a gRNA. In some embodiments, the KCNQ4 inhibitory RNA comprises a sequence according to SEQ ID NO: 42, SEQ ID NO: 43, SEQ ID NO: 44, SEQ ID NO: 45, SEQ ID NO: 46, SEQ ID NO: 47, SEQ ID NO: 48. In some embodiments, the promoter is an H1 or U6 promoter.

In some embodiments, one or more KCNQ4 inhibitory nucleic acids are engineered into a miR scaffold targeting region in a chimeric intron of a construct described herein. For example, in some embodiments, one, two, three, four, five, six, seven, eight, nine, ten, or more KCNQ4 inhibitory nucleic acids are engineered into the miR scaffold targeting region in the chimeric intron of a construct described herein. In some embodiments, one or more KCNQ4 inhibitory nucleic acids are engineered into a miR scaffold targeting region in a 3′ UTR of a construct described herein. For example, in some embodiments, one, two, three, four, five, six, seven, eight, nine, ten, or more KCNQ4 inhibitory nucleic acids are engineered into the miR scaffold targeting region in a 3′ UTR of a construct described herein.

In some embodiments, the present disclosure provides a construct comprising an inhibitory nucleic acid, wherein the inhibitory nucleic acid is or comprises one or more of miR1-155; miR2-155; miR4-155; miR5-155; miR6-155; miR7-155 miR1-16; miR1-26; miR1-96; miR1-122; miR1-135; miR1-182; miR1-183; miR1-335; miR1-451 and/or an miRNA selected from the group consisting of miR1-155; miR2-155; miR4-155; miR5-155; miR6-155; miR7-155; miR1-16; miR1-26; miR1-96; miR1-122; miR1-135; miR1-182; miR1-183; miR1-335; miR1-451 and combinations thereof. In some embodiments, the present disclosure provides a construct comprising an inhibitory nucleic acid, wherein the inhibitory nucleic acid is or comprises one or more of miR2-26; mir6-26; and combinations thereof.

In some embodiments, the present disclosure provides an AAV particle, further comprising a construct as provided herein. In some embodiments, the AAV particle further comprises an AAV capsid, wherein the AAV capsid is or is derived from an AAV2, AAV3, AAV4, AAV5, AAV6, AAV7, AAV8, AAV9, AAV10, AAV-rh8, AAV-rh10, AAV-rh39, AAV-rh43 or AAV Anc80 capsid. In some embodiments, the AAV capsid is an AAV Anc80 capsid.

In some embodiments, the present disclosure provides a composition comprising at least one construct provided herein. In some embodiments, the composition comprises an AAV particle as provided herein. In some embodiments, a particle of the composition further comprises an AAV capsid, wherein the AAV capsid is or is derived from an AAV2, AAV3, AAV4, AAV5, AAV6, AAV7, AAV8, AAV9, AAV10, AAV-rh8, AAV-rh10, AAV-rh39, AAV-rh43 or AAV Anc80 capsid. In some embodiments, the AAV capsid of the AAV particle is an AAV Anc80 capsid. In some embodiments, the composition is a pharmaceutical composition. In some embodiments, the composition further comprises a pharmaceutically acceptable carrier.

The present disclosure also provides a cell. In some embodiments, the cell comprises one or more constructs, compositions and/or particles as provided herein. In some embodiments, the cell is in vivo, ex vivo, or in vitro. In some embodiments, the cell is a mammalian cell. In some embodiments, the mammalian cell is a human cell. In some embodiments, the cell is immortalized to generate a stable cell line. In some embodiments, the human cell is in the ear of a subject. In some embodiments, the cell has at least one copy of an endogenous KCNQ4 gene has at least one sequence variation. In some embodiments, the at least one sequence variation results in a loss-of-function gene product.

In some embodiments, the present disclosure provides a cell comprising a coding sequence operably linked to a promoter, wherein the coding sequence encodes a Kv7.4 protein. In some embodiments, the present disclosure provides a cell comprising a coding sequence operably linked to a promoter, wherein the coding sequence encodes a loss-of-function KCNQ4 variant gene product. In some embodiments, a KCNQ4 gene product is a Kv7.4 protein. In some such embodiments, the present disclosure provides a population of cells comprising one or more cells, wherein the population is or comprises a stable cell line.

In some embodiments, the inner ear cell is an outer hair cell. In some embodiments, the inner ear cell is in the ear of a subject. In some embodiments, the inner ear cell is in vitro or ex vivo.

The present disclosure also provides a system. In some embodiments, the system comprises at least one composition as provided herein.

The present disclosure provides a method comprising contacting an inner ear cell with at least one composition as described herein.

The present disclosure provides a system, a method, or a kit comprising a device for as described in FIGS. 32-35.

The present disclosure provides a method comprising contacting an inner ear cell with at least one construct as provided herein and one or more plasmids comprising an AAV Rep gene, AAV Cap gene, AAV VA gene, AAV Eta gene, and AAV E4 gene.

In some embodiments, the inner ear cell is an outer hair cell. In some embodiments, the inner ear cell is in the ear of a subject. In some embodiments, the inner ear cell is in vitro or ex vivo.

The present disclosure provides a method comprising introducing at least one composition as provided herein into the inner ear of a subject. In some embodiments, the composition is introduced into the cochlea of the subject. In some embodiments, the composition is introduced via a round window membrane injection.

In some embodiments, a method of the present disclosure further comprises measuring a hearing level of the subject. In some embodiments, a hearing level is measured by performing an auditory brainstem response (ABR) test. In some embodiments, the method further comprises comparing the hearing level of the subject to a reference hearing level. In some embodiments, the reference hearing level is a published or historical reference hearing level. In some embodiments, the hearing level of the subject is measured after any construct provided herein, and the reference hearing level is a hearing level of the subject that was measured before any construct as provided herein was introduced. In some embodiments, the method further comprises measuring a level of a KCNQ4 gene product in a subject. In some embodiments, the level of the KCNQ4 gene product is measured in the inner ear of the subject. In some embodiments, the level of the KCNQ4 gene product is measured in the cochlea of the subject. In some embodiments, the method further comprises comparing the level of a KCNQ4 gene product in the subject to a reference KCNQ4 gene product level. In some embodiments, the reference hearing level is a published or historical reference KCNQ4 gene product level. In some embodiments, the level of a KCNQ4 gene product in the subject is measured after any construct as provided herein is introduced, and the reference KCNQ4 gene product level is a KCNQ4 gene product level of the subject that was measured before any composition as provided herein was introduced.

The present disclosure also provides a method of treating hearing loss comprising administering at least one composition as provided herein to a subject in need thereof. In some embodiments, the present disclosure provides a method of treating hearing loss comprising administering at least one particle as provided herein, to a subject in need thereof.

In some embodiments, any constructs as provided herein may be used in the treatment of hearing loss. In some embodiments, any composition as provided herein may be used in the treatment of hearing loss. In some embodiments, any particle as provided herein may be used in the treatment of hearing loss. In some embodiments, the present disclosure provides a use of a construct as provided herein for manufacture of a medicament to treat hearing loss. In some embodiments, the present disclosure provides a use of a composition as provided herein for the manufacture of a medicament to treat hearing loss. In some embodiments, the present disclosure provides a use of a particle as provided herein for the manufacture of a medicament to treat hearing loss.

### Definitions

The scope of the present disclosure is defined by the claims appended hereto and is not limited by certain embodiments described herein. Those skilled in the art, reading the present specification, will be aware of various modifications that may be equivalent to such described embodiments, or otherwise within the scope of the claims. In general, terms used herein are in accordance with their understood meaning in the art, unless clearly indicated otherwise. Explicit definitions of certain terms are provided below; meanings of these and other terms in particular instances throughout this specification will be clear to those skilled in the art from context.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

The articles “a” and “an,” as used herein, should be understood to include plural referents unless clearly indicated to the contrary. Claims or descriptions that include “or” between one or more members of a group are considered satisfied if one, more than one, or all of the group members are present in, employed in, or otherwise relevant to a given product or process unless indicated to the contrary or otherwise evident from the context. In some embodiments, exactly one member of a group is present in, employed in, or otherwise relevant to a given product or process. In some embodiments, more than one, or all group members are present in, employed in, or otherwise relevant to a given product or process. It is to be understood that the present disclosure encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists (e.g., in Markush group or similar format), it is to be understood that each subgroup of the elements is also disclosed, and any element(s) can be removed from the group. It should be understood that, in general, where embodiments or aspects are referred to as “comprising” particular elements, features, etc., certain embodiments or aspects “consist,” or “consist essentially of,” such elements, features, etc. For purposes of simplicity, those embodiments have not in every case been specifically set forth in so many words herein. It should also be understood that any embodiment or aspect can be explicitly excluded from the claims, regardless of whether the specific exclusion is recited in the specification.

Throughout the specification, whenever a polynucleotide or polypeptide is represented by a sequence of letters (e.g., A, C, G, and T, which denote adenosine, cytidine, guanosine, and thymidine, respectively, in the case of a polynucleotide), such polynucleotides or polypeptides are presented in 5′ to 3′ or N-terminus to C-terminus order, from left to right.

Administration: As used herein, the term “administration” typically refers to administration of a composition to a subject or system to achieve delivery of an agent to a subject or system. In some embodiments, an agent is, or is included in, a composition; in some embodiments, an agent is generated through metabolism of a composition or one or more components thereof. Those of ordinary skill in the art will be aware of a variety of routes that may, in appropriate circumstances, be utilized for administration to a subject, for example a human. For example, in some embodiments, administration may be systematic or local. In some embodiments, a systematic administration can be intravenous. In some embodiments, administration can be local. Local administration can involve delivery to cochlear perilymph via, e.g., injection through a round-window membrane or into scala-tympani, a scala-media injection through endolymph, perilymph and/or endolymph following canalostomy. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Allele: As used herein, the term “allele” refers to one of two or more existing genetic variants of a specific polymorphic genomic locus.

Amelioration: As used herein, the term “amelioration” refers to prevention, reduction or palliation of a state, or improvement of a state of a subject. Amelioration may include, but does not require, complete recovery or complete prevention of a disease, disorder or condition.

Amino acid: In its broadest sense, as used herein, the term “amino acid” refers to any compound and/or substance that can be incorporated into a polypeptide chain, e.g., through formation of one or more peptide bonds. In some embodiments, an amino acid has a general structure, e.g., H2N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than standard amino acids, regardless of whether it is prepared synthetically or obtained from a natural source. In some embodiments, an amino acid, including a carboxy- and/or amino-terminal amino acid in a polypeptide, can contain a structural modification as compared with general structure as shown above. For example, in some embodiments, an amino acid may be modified by methylation, amidation, acetylation, pegylation, glycosylation, phosphorylation, and/or substitution (e.g., of an amino group, a carboxylic acid group, one or more protons, and/or a hydroxyl group) as compared with a general structure. In some embodiments, such modification may, for example, alter circulating half-life of a polypeptide containing a modified amino acid as compared with one containing an otherwise identical unmodified amino acid. In some embodiments, such modification does not significantly alter a relevant activity of a polypeptide containing a modified amino acid, as compared with one containing an otherwise identical unmodified amino acid.

Approximately or About: As used herein, the terms “approximately” or “about” may be applied to one or more values of interest, including a value that is similar to a stated reference value. In some embodiments, the term “approximately” or “about” refers to a range of values that fall within ±10% (greater than or less than) of a stated reference value unless otherwise stated or otherwise evident from context (except where such number would exceed 100% of a possible value). For example, in some embodiments, the term “approximately” or “about” may encompass a range of values that within 10%, 9%, 8%, 7%, 6%, 5%, 4%, 3%, 2%, 1%, or less of a reference value.

Associated: As used herein, the term “associated” describes two events or entities as “associated” with one another, if the presence, level and/or form of one is correlated with that of the other. For example, a particular entity (e.g., polypeptide, genetic signature, metabolite, microbe, etc.) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Biologically active: As used herein, the term “biologically active” refers to an observable biological effect or result achieved by an agent or entity of interest. For example, in some embodiments, a specific binding interaction is a biological activity. In some embodiments, modulation (e.g., induction, enhancement, or inhibition) of a biological pathway or event is a biological activity. In some embodiments, presence or extent of a biological activity is assessed through detection of a direct or indirect product produced by a biological pathway or event of interest.

Characteristic portion: As used herein, the term “characteristic portion,” in the broadest sense, refers to a portion of a substance whose presence (or absence) correlates with presence (or absence) of a particular feature, attribute, or activity of the substance. In some embodiments, a characteristic portion of a substance is a portion that is found in a given substance and in related substances that share a particular feature, attribute or activity, but not in those that do not share the particular feature, attribute or activity. In some embodiments, a characteristic portion shares at least one functional characteristic with the intact substance. For example, in some embodiments, a “characteristic portion” of a protein or polypeptide is one that contains a continuous stretch of amino acids, or a collection of continuous stretches of amino acids, that together are characteristic of a protein or polypeptide. In some embodiments, each such continuous stretch generally contains at least 2, 5, 10, 15, 20, 50, or more amino acids. In general, a characteristic portion of a substance (e.g., of a protein, antibody, etc.) is one that, in addition to a sequence and/or structural identity specified above, shares at least one functional characteristic with the relevant intact substance. In some embodiments, a characteristic portion may be biologically active.

Characteristic sequence: As used herein, the term “characteristic sequence” is a sequence that is found in all members of a family of polypeptides or nucleic acids, and therefore can be used by those of ordinary skill in the art to define members of the family.

Characteristic sequence element: As used herein, the phrase “characteristic sequence element” refers to a sequence element found in a polymer (e.g., in a polypeptide or nucleic acid) that represents a characteristic portion of that polymer. In some embodiments, presence of a characteristic sequence element correlates with presence or level of a particular activity or property of a polymer. In some embodiments, presence (or absence) of a characteristic sequence element defines a particular polymer as a member (or not a member) of a particular family or group of such polymers. A characteristic sequence element typically comprises at least two monomers (e.g., amino acids or nucleotides). In some embodiments, a characteristic sequence element includes at least 2, 3, 4, 5, 6, 7, 8, 9, 10, 11, 12, 13, 14, 15, 20, 25, 30, 35, 40, 45, 50, or more monomers (e.g., contiguously linked monomers). In some embodiments, a characteristic sequence element includes at least first and second stretches of contiguous monomers spaced apart by one or more spacer regions whose length may or may not vary across polymers that share a sequence element.

Cleavage: As used herein, the term “cleavage” refers to generation of a break in DNA. For example, in some embodiments, cleavage could refer to either a single-stranded break or a double-stranded break depending on a type of nuclease that may be employed to cause such a break.

Combination therapy: As used herein, the term “combination therapy” refers to those situations in which a subject is simultaneously exposed to two or more therapeutic regimens (e.g., two or more therapeutic agents). In some embodiments, two or more agents may be administered simultaneously. In some embodiments, two or more agents may be administered sequentially. In some embodiments, two or more agents may be administered in overlapping dosing regimens.

Comparable: As used herein, the term “comparable” refers to two or more agents, entities, situations, sets of conditions, subjects, populations, etc., that may not be identical to one another but that are sufficiently similar to permit comparison therebetween so that one skilled in the art will appreciate that conclusions may reasonably be drawn based on differences or similarities observed. In some embodiments, comparable sets of agents, entities, situations, sets of conditions, subjects, populations, etc. are characterized by a plurality of substantially identical features and one or a small number of varied features. Those of ordinary skill in the art will understand, in context, what degree of identity is required in any given circumstance for two or more such agents, entities, situations, sets of conditions, subjects, populations, etc. to be considered comparable. For example, those of ordinary skill in the art will appreciate that sets of agents, entities, situations, sets of conditions, subjects, populations, etc. are comparable to one another when characterized by a sufficient number and type of substantially identical features to warrant a reasonable conclusion that differences in results obtained or phenomena observed under or with different sets of circumstances, stimuli, agents, entities, situations, sets of conditions, subjects, populations, etc. are caused by or indicative of the variation in those features that are varied.

Construct: As used herein, the term “construct” refers to a composition including a polynucleotide capable of carrying at least one heterologous polynucleotide. In some embodiments, a construct can be a plasmid, a transposon, a cosmid, an artificial chromosome (e.g., a human artificial chromosome (HAC), a yeast artificial chromosome (YAC), a bacterial artificial chromosome (BAC), or a P1-derived artificial chromosome (PAC)) or a viral construct, and any Gateway® plasmids. A construct can, e.g., include sufficient cis-acting elements for expression; other elements for expression can be supplied by the host primate cell or in an in vitro expression system. A construct may include any genetic element (e.g., a plasmid, a transposon, a cosmid, an artificial chromosome, or a viral construct, etc.) that is capable of replicating when associated with proper control elements. Thus, in some embodiments, “construct” may include a cloning and/or expression construct and/or a viral construct (e.g., an adeno-associated virus (AAV) construct, an adenovirus construct, a lentivirus construct, or a retrovirus construct).

Conservative: As used herein, the term “conservative” refers to instances describing a conservative amino acid substitution, including a substitution of an amino acid residue by another amino acid residue having a side chain R group with similar chemical properties (e.g., charge or hydrophobicity). In general, a conservative amino acid substitution will not substantially change functional properties of interest of a protein, for example, ability of a receptor to bind to a ligand. Examples of groups of amino acids that have side chains with similar chemical properties include: aliphatic side chains such as glycine (Gly, G), alanine (Ala, A), valine (Val, V), leucine (Leu, L), and isoleucine (Ile, I); aliphatic-hydroxyl side chains such as serine (Ser, S) and threonine (Thr, T); amide-containing side chains such as asparagine (Asn, N) and glutamine (Gln, Q); aromatic side chains such as phenylalanine (Phe, F), tyrosine (Tyr, Y), and tryptophan (Trp, W); basic side chains such as lysine (Lys, K), arginine (Arg, R), and histidine (His, H); acidic side chains such as aspartic acid (Asp, D) and glutamic acid (Glu, E); and sulfur-containing side chains such as cysteine (Cys, C) and methionine (Met, M). Conservative amino acids substitution groups include, for example, valine/leucine/isoleucine (Val/Leu/Ile, V/L/I), phenylalanine/tyrosine (Phe/Tyr, F/Y), lysine/arginine (Lys/Arg, K/R), alanine/valine (Ala/Val, A/V), glutamate/aspartate (Glu/Asp, E/D), and asparagine/glutamine (Asn/Gln, N/Q). In some embodiments, a conservative amino acid substitution can be a substitution of any native residue in a protein with alanine, as used in, for example, alanine scanning mutagenesis. In some embodiments, a conservative substitution is made that has a positive value in the PAM250 log-likelihood matrix disclosed in Gonnet et al., 1992, Science 256:1443-1445, which is incorporated herein by reference in its entirety. In some embodiments, a substitution is a moderately conservative substitution wherein the substitution has a nonnegative value in the PAM250 log-likelihood matrix. One skilled in the art would appreciate that a change (e.g., substitution, addition, deletion, etc.) of amino acids that are not conserved between the same protein from different species is less likely to have an effect on the function of a protein and therefore, these amino acids should be selected for mutation. Amino acids that are conserved between the same protein from different species should not be changed (e.g., deleted, added, substituted, etc.), as these mutations are more likely to result in a change in function of a protein.

CONSERVATIVE AMINO ACID SUBSTITUTIONS

| For | | |
|---|---|---|
| Amino Acid | Code | Replace With |
| Alanine | A | D-ala, Gly, Aib, β-Ala, Acp, L-Cys, D-Cys |
| Arginine | R | D-Arg, Lys, D-Lys, homo-Arg, D-homo-Arg, |
| | | Met, Ile, D-Met, D-Ile, Orn, D-Orn |
| Asparagine | N | D-Asn, Asp, D-Asp, Glu, D-Glu, Gln, D-Gln |
| Aspartic | D | D-Asp, D-Asn, Asn, Glu, D-Glu, Gln, D-Gln |
| Acid | | |
| Cysteine | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic | E | D-Glu, D-Asp, Asp, Asn, D-Asn, Gln, D-Gln |
| Acid | | |
| Glycine | G | Ala, D-Ala, Pro, D-Pro, Aib, β-Ala, Acp |
| Isoleucine | I | D-Ile, Val, D-Val, AdaA, AdaG, Leu, D-Leu, |
| | | Met, D-Met |
| Leucine | L | D-Leu, Val, D-Val, AdaA, AdaG, Leu, D-Leu, |
| | | Met, D-Met |
| Lysine | K | D-Lys, Arg, D-Arg, homo-Arg, D-homo-Arg, |
| | | Met, D-Met, Ile, D-Ile, Orn, D-Orn |
| Methionine | M | D-Met, S-Me-Cys, Ile, D-Ile, Leu, D-Leu, |
| | | Val, D-Val |
| Phenylalanine | F | D-Phe, Tyr, D-Thr, L-Dopa, His, D-His, Trp, |
| | | D-Trp, Trans-3,4 or 5-phenylproline, AdaA, |
| | | AdaG, cis-3,4 or 5-phenylproline, Bpa, D-Bpa |
| Proline | P | D-Pro, L-I-thioazolidine-4-carboxylic acid, |
| | | D-or-L-1-oxazolidine-4-carboxylic acid |
| | | (Kauer, U.S. Pat. No. 4,511,390) |
| Serine | S | D-Ser, Thr, D-Thr, allo-Thr, Met, D-Met, |
| | | Met (O), D-Met (O), L-Cys, D-Cys |
| Threonine | T | D-Thr, Ser, D-Ser, allo-Thr, Met, D-Met, |
| | | Met (O), D-Met (O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met, |
| | | AdaA, AdaG |

Control: As used herein, the term “control” refers to the art-understood meaning of a “control” being a standard against which results are compared. Typically, controls are used to augment integrity in experiments by isolating variables in order to make a conclusion about such variables. In some embodiments, a control is a reaction or assay that is performed simultaneously with a test reaction or assay to provide a comparator. For example, in one experiment, a “test” (i.e., a variable being tested) is applied. In a second experiment, a “control,” the variable being tested is not applied. In some embodiments, a control is a historical control (e.g., of a test or assay performed previously, or an amount or result that is previously known). In some embodiments, a control is or comprises a printed or otherwise saved record. In some embodiments, a control is a positive control. In some embodiments, a control is a negative control.

Determining, measuring, evaluating, assessing, assaying and analyzing: As used herein, the terms “determining,” “measuring,” “evaluating,” “assessing,” “assaying,” and “analyzing” may be used interchangeably to refer to any form of measurement, and include determining if an element is present or not. These terms include both quantitative and/or qualitative determinations. Assaying may be relative or absolute. For example, in some embodiments, “Assaying for the presence of can be determining an amount of something present and/or determining whether or not it is present or absent.

Editing: As used herein, the term “edit,” “editing,” or “edited” refers to a method of altering a nucleic acid sequence of a polynucleotide (e.g., a wild type naturally occurring nucleic acid sequence or a mutated naturally occurring sequence) by selective deletion of a specific nucleic acid sequence (e.g., a genomic target sequence), a given specific inclusion of new sequence through use of an exogenous nucleic acid sequence, or a replacement of nucleic acid sequence with an exogenous nucleic acid sequence. In some embodiments, such a specific genomic target includes, but may be not limited to, a chromosomal region, mitochondrial DNA, a gene, a promoter, an open reading frame or any nucleic acid sequence.

Engineered: In general, as used herein, the term “engineered” refers to an aspect of having been manipulated by the hand of man. For example, a cell or organism is considered to be “engineered” if it has been manipulated so that its genetic information is altered (e.g., new genetic material not previously present has been introduced, for example by transformation, mating, somatic hybridization, transfection, transduction, or other mechanism, or previously present genetic material is altered or removed, for example by substitution or deletion mutation, or by mating protocols). As is common practice and is understood by those in the art, progeny of an engineered polynucleotide or cell are typically still referred to as “engineered” even though the actual manipulation was performed on a prior entity.

Excipient: As used herein, the term “excipient” refers to an inactive (e.g., non-therapeutic) agent that may be included in a pharmaceutical composition, for example to provide or contribute to a desired consistency or stabilizing effect. In some embodiments, suitable pharmaceutical excipients may include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Expression: As used herein, the term “expression” of a nucleic acid sequence refers to generation of any gene product (e.g., transcript, e.g., mRNA, e.g., polypeptide, etc.) from a nucleic acid sequence. In some embodiments, a gene product can be a transcript. In some embodiments, a gene product can be a polypeptide. In some embodiments, expression of a nucleic acid sequence involves one or more of the following: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Functional: As used herein, the term “functional” describes something that exists in a form in which it exhibits a property and/or activity by which it is characterized. For example, in some embodiments, a “functional” biological molecule is a biological molecule in a form in which it exhibits a property and/or activity by which it is characterized. In some such embodiments, a functional biological molecule is characterized relative to another biological molecule which is non-functional in that the “non-functional” version does not exhibit the same or equivalent property and/or activity as the “functional” molecule. A biological molecule may have one function, two functions (i.e., bifunctional) or many functions (i.e., multifunctional).

Gene: As used herein, the term “gene” refers to a DNA sequence in a chromosome that codes for a gene product (e.g., an RNA product, e.g., a polypeptide product). In some embodiments, a gene includes coding sequence (i.e., sequence that encodes a particular product). In some embodiments, a gene includes non-coding sequence. In some particular embodiments, a gene may include both coding (e.g., exonic) and non-coding (e.g., intronic) sequence. In some embodiments, a gene may include one or more regulatory sequences (e.g., promoters, enhancers, etc.) and/or intron sequences that, for example, may control or impact one or more aspects of gene expression (e.g., cell-type-specific expression, inducible expression, etc.). As used herein, the term “gene” generally refers to a portion of a nucleic acid that encodes a polypeptide or fragment thereof; the term may optionally encompass regulatory sequences, as will be clear from context to those of ordinary skill in the art. This definition is not intended to exclude application of the term “gene” to non-protein-coding expression units but rather to clarify that, in most cases, the term as used in this document refers to a polypeptide-coding nucleic acid. In some embodiments, a gene may encode a polypeptide, but that polypeptide may not be functional, e.g., a gene variant may encode a polypeptide that does not function in the same way, or at all, relative to the wild-type gene. In some embodiments, a gene may encode a transcript which, in some embodiments, may be toxic beyond a threshold level. In some embodiments, a gene may encode a polypeptide, but that polypeptide may not be functional and/or may be toxic beyond a threshold level.

Genome Editing System: As used herein, the term “genome editing system” refers to any system having DNA editing activity. Among other things, DNA editing activity can include deleting, replacing, or inserting a DNA sequence in a genome. In some embodiments, a genome editing system comprises RNA-guided DNA editing activity. In some embodiments, a genome editing system of the present disclosure includes more than one component. In some embodiments, a genome editing system includes at least two components adapted from naturally occurring CRISPR systems: a guide RNA (gRNA) and an RNA-guided nuclease. In certain embodiments, these two components form a complex that is capable of associating with a specific nucleic acid sequence and editing DNA in or around that nucleic acid sequence, for instance by making one or more of a single-strand break (an SSB or nick), a double-strand break (a DSB) and/or a point mutation. In some embodiments, genome editing systems of the present disclosure lack a component having cleavage activity but maintain a component(s) having DNA binding activity. In some such embodiments, a genome editing system of the present disclosure comprises a component(s) that functions as an inhibitor of DNA activity, e.g., transcription, translation, etc. In some embodiments, a genome editing system of the present disclosure comprises a component(s) fused to modulators to modulate target DNA expression.

Genomic modification: As used herein, the term “genomic modification” refers to a change made in a genomic region of a cell that permanently alters a genome (e.g., an endogenous genome) of that cell. In some embodiments, such changes are in vitro, ex vivo, or in vivo. In some embodiments, every cell in a living organism is modified. In some embodiments, only a particular set of cells such as, e.g., in a specific organ, is modified. For example, in some embodiments, a genome is modified by deletion, substitution, or addition of one or more nucleotides from one or more genomic regions. In some embodiments, a genomic modification is performed in a stem cell or undifferentiated cell. In some such embodiments, progeny of a genomically modified cell or organism will also be genomically modified, relative to a parental genome prior to modification. In some embodiments, a genomic modification is performed on a mature or post-mitotic cell such that no progeny will be generated and thus, no genomic modifications propagated other than in a particular cell.

Hearing loss: As used herein, the term “hearing loss” may be used to a partial or total inability of a living organism to hear. In some embodiments, hearing loss may be acquired. In some embodiments, hearing loss may be hereditary. In some embodiments, hearing loss may be genetic. In some embodiments, hearing loss may be as a result of disease or trauma (e.g., physical trauma, treatment with one or more agents resulting in hearing loss, etc.). In some embodiments, hearing loss may be due to one or more known genetic causes and/or syndromes. In some embodiments, hearing loss may be of unknown etiology. In some embodiments, hearing loss may or may not be mitigated by use of hearing aids or other treatments.

Heterologous: As used herein, the term “heterologous” may be used in reference to one or more regions of a particular molecule as compared to another region and/or another molecule. For example, in some embodiments, heterologous polypeptide domains, refers to the fact that polypeptide domains do not naturally occur together (e.g., in the same polypeptide). For example, in fusion proteins generated by the hand of man, a polypeptide domain from one polypeptide may be fused to a polypeptide domain from a different polypeptide. In such a fusion protein, two polypeptide domains would be considered “heterologous” with respect to each other, as they do not naturally occur together.

Identity: As used herein, the term “identity” refers to overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of percent identity of two nucleic acid or polypeptide sequences, for example, can be performed by aligning two sequences for optimal comparison purposes (e.g., gaps can be introduced in one or both of a first and a second sequences for optimal alignment and non-identical sequences can be disregarded for comparison purposes). In some embodiments, a length of a sequence aligned for comparison purposes is at least 30%, at least 40%, at least 50%, at least 60%, at least 70%, at least 80%, at least 90%, at least 95%, or substantially 100% of length of a reference sequence; nucleotides at corresponding positions are then compared. When a position in the first sequence is occupied by the same residue (e.g., nucleotide or amino acid) as a corresponding position in the second sequence, then the two molecules (i.e., first and second) are identical at that position. Percent identity between two sequences is a function of the number of identical positions shared by the two sequences being compared, taking into account the number of gaps, and the length of each gap, which needs to be introduced for optimal alignment of the two sequences. Comparison of sequences and determination of percent identity between two sequences can be accomplished using a mathematical algorithm. For example, percent identity between two nucleotide sequences can be determined using the algorithm of Meyers and Miller (CABIOS, 1989, 4: 11-17, which is herein incorporated by reference in its entirety), which has been incorporated into the ALIGN program (version 2.0). In some embodiments, nucleic acid sequence comparisons made with the ALIGN program use a PAM120 weight residue table, a gap length penalty of 12 and a gap penalty of 4.

Inhibitory nucleic acid: As used herein, the term “inhibitory nucleic acid” refers to a nucleic acid sequence that hybridizes specifically to a target gene, including target DNA or RNA (e.g., a target mRNA (e.g., a potassium channel gene product, e.g., a potassium channel mRNA, e.g., KCNQ4 mRNA)). Thereby, in some embodiments, an inhibitory nucleic acid inhibits expression and/or activity of a target gene. In some embodiments, an inhibitory nucleic acid is a short interfering RNA (siRNA), a short hairpin RNA (shRNA), a microRNA (or “miRNA”), an antisense oligonucleotide, a guide RNA (gRNA), or a ribozyme. In some embodiments, an inhibitory nucleic acid is between about 10 nucleotides to about 30 nucleotides in length (e.g., about 10 nucleotides to about 28 nucleotides, about 10 nucleotides to about 26 nucleotides, about 10 nucleotides to about 24 nucleotides, about 10 nucleotides to about 22 nucleotides, about 10 nucleotides to about 20 nucleotides, about 10 nucleotides to about 18 nucleotides, about 10 nucleotides to about 16 nucleotides, about 10 nucleotides to about 14 nucleotides, about 10 nucleotides to about 12 nucleotides, about 12 nucleotides to about 30 nucleotides, about 12 nucleotides to about 28 nucleotides, about 12 nucleotides to about 26 nucleotides, about 12 nucleotides to about 24 nucleotides, about 12 nucleotides to about 22 nucleotides, about 12 nucleotides to about 20 nucleotides, about 12 nucleotides to about 18 nucleotides, about 12 nucleotides to about 16 nucleotides, about 12 nucleotides to about 14 nucleotides, about 16 nucleotides to about 30 nucleotides, about 16 nucleotides to about 28 nucleotides, about 16 nucleotides to about 26 nucleotides, about 16 nucleotides to about 24 nucleotides, about 16 nucleotides to about 22 nucleotides, about 16 nucleotides to about 20 nucleotides, about 16 nucleotides to about 18 nucleotides, about 18 nucleotides to about 30 nucleotides, about 18 nucleotides to about 28 nucleotides, about 18 nucleotides to about 26 nucleotides, about 18 nucleotides to about 24 nucleotides, about 18 nucleotides to about 22 nucleotides, about 18 nucleotides to about 20 nucleotides, about 20 nucleotides to about 30 nucleotides, about 20 nucleotides to about 28 nucleotides, about 20 nucleotides to about 26 nucleotides, about 20 nucleotides to about 24 nucleotides, about 20 nucleotides to about 22 nucleotides, about 22 nucleotides to about 30 nucleotides, about 22 nucleotides to about 28 nucleotides, about 22 nucleotides to about 26 nucleotides, about 22 nucleotides to about 24 nucleotides, about 24 nucleotides to about 30 nucleotides, about 24 nucleotides to about 28 nucleotides, about 24 nucleotides to about 26 nucleotides, about 26 nucleotides to about 30 nucleotides, about 26 nucleotides to about 28 nucleotides, about 28 nucleotides to about 30 nucleotides, or 11, 12, 13, 14, 15, 16, 17, 18, 19, 20, 21, 22, 23, 24, 25, 26, 27, 28, 29 or 30 nucleotides). In some embodiments, an inhibitory nucleic acid is an inhibitory RNA that targets KCNQ4. In some such embodiments, an inhibitory KCNQ4 RNA hybridizes specifically to a target on a KCNQ4. In some such embodiments, a KCNQ4 inhibitory RNA includes, e.g., an inhibitory nucleic acid is a short interfering RNA (siRNA), a short hairpin RNA (shRNA), a microRNA (miRNA), an antisense oligonucleotide, a guide RNA (gRNA), or a ribozyme. In some embodiments, hybridizing of an inhibitory KCNQ4 RNA reduces expression of a KCNQ4 gene product. Exemplary KCNQ4 inhibitory RNA sequences are provided herein.

Improve, increase, enhance, inhibit or reduce: As used herein, the terms “improve,” “increase,” “enhance,” “inhibit,” “reduce,” or grammatical equivalents thereof, indicate values that are relative to a baseline or other reference measurement. In some embodiments, a value is statistically significantly difference that a baseline or other reference measurement. In some embodiments, an appropriate reference measurement may be or comprise a measurement in a particular system (e.g., in a single individual) under otherwise comparable conditions absent presence of (e.g., prior to and/or after) a particular agent or treatment, or in presence of an appropriate comparable reference agent. In some embodiments, an appropriate reference measurement may be or comprise a measurement in comparable system known or expected to respond in a particular way, in presence of the relevant agent or treatment. In some embodiments, an appropriate reference is a negative reference; in some embodiments, an appropriate reference is a positive reference.

Knockdown: As used herein, the term “knockdown” refers to a decrease in expression of one or more gene products. In some embodiments, an inhibitory nucleic acid achieve knockdown. In some embodiments, a genome editing system described herein achieves knockdown.

Knockout: As used herein, the term “knockout” refers to ablation of expression of one or more gene products. In some embodiments, a genome editing system described herein achieve knockout.

Modulating: As used herein, the term “modulating,” means mediating a detectable increase or decrease in a level of a response in a subject compared with a level of a response in a subject in absence of a treatment or compound, and/or compared with a level of a response in an otherwise identical but untreated subject. The term encompasses perturbing and/or affecting a native signal or response thereby mediating a beneficial therapeutic response in a subject, preferably, a human.

Nuclease: As used herein, the term “nuclease” refers to an agent, for example a protein or a small molecule, capable of cleaving a phosphodiester bond connecting nucleotide residues in a nucleic acid molecule. In some embodiments, a nuclease is a protein, e.g., an enzyme that can bind a nucleic acid molecule and cleave a phosphodiester bond connecting nucleotide residues within the nucleic acid molecule. A nuclease may be an endonuclease, cleaving a phosphodiester bonds within a polynucleotide chain, or an exonuclease, cleaving a phosphodiester bond at the end of the polynucleotide chain. In some embodiments, a nuclease is a site-specific nuclease, binding and/or cleaving a specific phosphodiester bond within a specific nucleotide sequence, which is also referred to herein as the “recognition sequence,” the “nuclease target site,” or the “target site.” In some embodiments, a nuclease is a RNA-guided (i.e., RNA-programmable) nuclease, which complexes with (e.g., binds with) an RNA having a sequence that complements a target site, thereby providing the sequence specificity of a nuclease. In some embodiments, a nuclease recognizes a single stranded target site, while in other embodiments, a nuclease recognizes a double-stranded target site, for example a double-stranded DNA target site. Target sites of many naturally occurring nucleases, for example, many naturally occurring DNA restriction nucleases, are well known to those of skill in the art. In many cases, a DNA nuclease, such as EcoRI, HindIII, or BamHI, recognize a palindromic, double-stranded DNA target site of 4 to 10 base pairs in length, and cut each of the two DNA strands at a specific position within a target site. Some endonucleases cut a double-stranded nucleic acid target site symmetrically, i.e., cutting both strands at the same position so that the ends comprise base-paired nucleotides, also referred to herein as blunt ends. Other endonucleases cut a double-stranded nucleic acid target sites asymmetrically, i.e., cutting each strand at a different position so that the ends comprise unpaired nucleotides. Unpaired nucleotides at an end of a double-stranded DNA molecule are also referred to as “overhangs,” e.g., as “5′-overhang” or as “3′-overhang,” depending on whether unpaired nucleotide(s) form(s) the 5′ or the 3′ end of a given DNA strand. Double-stranded DNA molecule ends ending with unpaired nucleotide(s) are also referred to as sticky ends, as they can “stick to” other double-stranded DNA molecule ends comprising complementary unpaired nucleotide(s). A nuclease protein typically comprises a “binding domain” that mediates interaction of a protein with a nucleic acid substrate, and also, in some cases, specifically binds to a target site, and a “cleavage domain” that catalyzes the cleavage of a phosphodiester bond within a nucleic acid backbone. In some embodiments, a nuclease protein can bind and cleave a nucleic acid molecule in a monomeric form, while, in other embodiments, a nuclease protein has to dimerize or multimerize in order to cleave a target nucleic acid molecule. Binding domains and cleavage domains of naturally occurring nucleases, as well as modular binding domains and cleavage domains that can be fused to create nucleases binding specific target sites, are well known to those of skill in the art.

Nucleic acid: As used herein, the term “nucleic acid”, in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid residue (e.g., a nucleotide and/or nucleoside); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 1 10, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is partly or wholly single stranded; in some embodiments, a nucleic acid is partly or wholly double stranded. In some embodiments, a nucleic acid has a nucleotide sequence comprising at least one element that encodes, or is complementary to a sequence that encodes, a polypeptide. In some embodiments, a nucleic acid has enzymatic activity.

Operably linked: As used herein, refers to a juxtaposition wherein the components described are in a relationship permitting them to function in their intended manner. A control element “operably linked” to a functional element is associated in such a way that expression and/or activity of the functional element is achieved under conditions compatible with the control element. In some embodiments, “operably linked” control elements are contiguous (e.g., covalently linked) with coding elements of interest; in some embodiments, control elements act in trans to or otherwise at a from the functional element of interest. In some embodiments, “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. In some embodiments, for example, a functional linkage may include transcriptional control. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

Pharmaceutical composition: As used herein, the term “pharmaceutical composition” refers to a composition in which an active agent is formulated together with one or more pharmaceutically acceptable carriers. In some embodiments, an active agent is present in unit dose amount appropriate for administration in a therapeutic regimen that shows a statistically significant probability of achieving a predetermined therapeutic effect when administered to a relevant population. In some embodiments, a pharmaceutical composition may be specially formulated for administration in solid or liquid form, including those adapted for, e.g., administration, for example, an injectable formulation that is, e.g., an aqueous or non-aqueous solution or suspension or a liquid drop designed to be administered into an ear canal. In some embodiments, a pharmaceutical composition may be formulated for administration via injection either in a particular organ or compartment, e.g., directly into an ear, or systemic, e.g., intravenously. In some embodiments, a formulation may be or comprise drenches (aqueous or non-aqueous solutions or suspensions), tablets, boluses, powders, granules, pastes, capsules, powders, etc. In some embodiments, an active agent may be or comprise an isolated, purified, or pure compound.

Pharmaceutically acceptable: As used herein, the term “pharmaceutically acceptable” which, for example, may be used in reference to a carrier, diluent, or excipient used to formulate a pharmaceutical composition as disclosed herein, means that a carrier, diluent, or excipient is compatible with other ingredients of a composition and not deleterious to a recipient thereof.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting a subject compound from one organ, or portion of a body, to another organ, or portion of a body. Each carrier must be is “acceptable” in the sense of being compatible with other ingredients of a formulation and not injurious to a patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Polypeptide: As used herein, the term “polypeptide” refers to any polymeric chain of residues (e.g., amino acids) that are typically linked by peptide bonds. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may include one or more pendant groups or other modifications, e.g., modifying or attached to one or more amino acid side chains, at a polypeptide's N-terminus, at a polypeptide's C-terminus, or any combination thereof. In some embodiments, such pendant groups or modifications may be acetylation, amidation, lipidation, methylation, pegylation, etc., including combinations thereof. In some embodiments, polypeptides may contain L-amino acids, D-amino acids, or both and may contain any of a variety of amino acid modifications or analogs known in the art. In some embodiments, useful modifications may be or include, e.g., terminal acetylation, amidation, methylation, etc. In some embodiments, a protein may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. In some embodiments, a protein is antibodies, antibody fragments, biologically active portions thereof, and/or characteristic portions thereof.

Polynucleotide: As used herein, the term “polynucleotide” refers to any polymeric chain of nucleic acids. In some embodiments, a polynucleotide is or comprises RNA; in some embodiments, a polynucleotide is or comprises DNA. In some embodiments, a polynucleotide is, comprises, or consists of one or more natural nucleic acid residues. In some embodiments, a polynucleotide is, comprises, or consists of one or more nucleic acid analogs. In some embodiments, a polynucleotide analog differs from a nucleic acid in that it does not utilize a phosphodiester backbone. Alternatively or additionally, in some embodiments, a polynucleotide has one or more phosphorothioate and/or 5′-N-phosphoramidite linkages rather than phosphodiester bonds. In some embodiments, a polynucleotide is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a polynucleotide is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a polynucleotide comprises one or more modified sugars (e.g., 2′-fluororibose, ribose, 2′-deoxyribose, arabinose, and hexose) as compared with those in natural nucleic acids. In some embodiments, a polynucleotide has a nucleotide sequence that encodes a functional gene product such as an RNA or protein. In some embodiments, a polynucleotide includes one or more introns. In some embodiments, a polynucleotide is prepared by one or more of isolation from a natural source, enzymatic synthesis by polymerization based on a complementary template (in vivo or in vitro), reproduction in a recombinant cell or system, and chemical synthesis. In some embodiments, a polynucleotide is at least 3, 4, 5, 6, 7, 8, 9, 10, 15, 20, 25, 30, 35, 40, 45, 50, 55, 60, 65, 70, 75, 80, 85, 90, 95, 100, 1 10, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a polynucleotide is partly or wholly single stranded; in some embodiments, a polynucleotide is partly or wholly double stranded. In some embodiments, a polynucleotide has a nucleotide sequence comprising at least one element that encodes, or is the complement of a sequence that encodes, a polypeptide. In some embodiments, a polynucleotide has enzymatic activity.

Protein: As used herein, the term “protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means.

Recombinant: As used herein, the term “recombinant” is intended to refer to polypeptides that are designed, engineered, prepared, expressed, created, manufactured, and/or or isolated by recombinant means, such as polypeptides expressed using a recombinant expression construct transfected into a host cell; polypeptides isolated from a recombinant, combinatorial human polypeptide library; polypeptides isolated from an animal (e.g., a mouse, rabbit, sheep, fish, etc.) that is transgenic for or otherwise has been manipulated to express a gene or genes, or gene components that encode and/or direct expression of the polypeptide or one or more component(s), portion(s), element(s), or domain(s) thereof; and/or polypeptides prepared, expressed, created or isolated by any other means that involves splicing or ligating selected nucleic acid sequence elements to one another, chemically synthesizing selected sequence elements, and/or otherwise generating a nucleic acid that encodes and/or directs expression of a polypeptide or one or more component(s), portion(s), element(s), or domain(s) thereof. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source such as, for example, in the germline of a source organism of interest (e.g., of a human, a mouse, etc.).

Reference: As used herein, the term “reference” describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control. In some embodiments, a reference is a negative control reference; in some embodiments, a reference is a positive control reference.

Regulatory Element: As used herein, the term “regulatory element” or “regulatory sequence” refers to non-coding regions of DNA that regulate, in some way, expression of one or more particular genes. In some embodiments, such genes are apposed or “in the neighborhood” of a given regulatory element. In some embodiments, such genes are located quite far from a given regulatory element. In some embodiments, a regulatory element impairs or enhances transcription of one or more genes. In some embodiments, a regulatory element may be located in cis to a gene being regulated. In some embodiments, a regulatory element may be located in trans to a gene being regulated. For example, in some embodiments, a regulatory sequence refers to a nucleic acid sequence which is regulates expression of a gene product operably linked to a regulatory sequence. In some such embodiments, this sequence may be an enhancer sequence and other regulatory elements which regulate expression of a gene product.

Sample: As used herein, the term “sample” typically refers to an aliquot of material obtained or derived from a source of interest. In some embodiments, a source of interest is a biological or environmental source. In some embodiments, a source of interest may be or comprise a cell or an organism, such as a microbe (e.g., virus), a plant, or an animal (e.g., a human). In some embodiments, a source of interest is or comprises biological tissue or fluid. In some embodiments, a biological tissue or fluid may be or comprise amniotic fluid, aqueous humor, ascites, bile, bone marrow, blood, breast milk, cerebrospinal fluid, cerumen, chyle, chime, ejaculate, endolymph, exudate, feces, gastric acid, gastric juice, lymph, mucus, pericardial fluid, perilymph, peritoneal fluid, pleural fluid, pus, rheum, saliva, sebum, semen, serum, smegma, sputum, synovial fluid, sweat, tears, urine, vaginal secretions, vitreous humour, vomit, and/or combinations or component(s) thereof. In some embodiments, a biological fluid may be or comprise an intracellular fluid, an extracellular fluid, an intravascular fluid (blood plasma), an interstitial fluid, a lymphatic fluid, and/or a transcellular fluid. In some embodiments, a biological fluid may be or comprise a plant exudate. In some embodiments, a biological tissue or sample may be obtained, for example, by aspirate, biopsy (e.g., fine needle or tissue biopsy), swab (e.g., oral, nasal, skin, or vaginal swab), scraping, surgery, washing or lavage (e.g., bronchioalveolar, ductal, nasal, ocular, oral, uterine, vaginal, or other washing or lavage). In some embodiments, a biological sample is or comprises cells obtained from an individual. In some embodiments, a sample is a “primary sample” obtained directly from a source of interest by any appropriate means. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to one or more techniques such as amplification or reverse transcription of nucleic acid, isolation and/or purification of certain components, etc.

Subject: As used herein, the term “subject” refers an organism, typically a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

Substantially: As used herein, the term “substantially” refers to a qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result. The term “substantially” is therefore used herein to capture a potential lack of completeness inherent in many biological and chemical phenomena.

Target site: As used herein, the term “target site” means a portion of a nucleic acid to which a binding molecule, e.g., a microRNA, an siRNA, a guide RNA (“gRNA”) or a guide RNA:Cas complex, will bind, provided sufficient conditions for binding exist. In some embodiments, a nucleic acid comprising a target site is double stranded. In some embodiments, a nucleic acid comprising a target site is single stranded. Typically, a target site comprises a nucleic acid sequence to which a binding molecule, e.g., a gRNA or a gRNA:Cas complex described herein, binds and/or that is cleaved as a result of such binding. In some embodiments, a target site comprises a nucleic acid sequence (also referred to herein as a target sequence or protospacer) that is complementary to a DNA sequence to which the targeting sequence (also referred to herein as the spacer) of a gRNA described herein binds. In some embodiments in the context of RNA-guided nucleases, e.g., CRISPR/Cas nucleases, a target site typically comprises a nucleotide sequence (also referred to herein as a target sequence or a protospacer) that is complementary to a sequence comprised in a gRNA (also referred to herein as the targeting sequence or the spacer) of an RNA-programmable nuclease. In some such embodiments, a target site further comprises a protospacer adjacent motif (PAM) at the 3′ end or 5′ end adjacent to the gRNA-complementary sequence. For an RNA-guided nuclease Cas9, a target sequence may be, in some embodiments, 16-24 base pairs plus a 3-6 base pair PAM (e.g., NNN, wherein N represents any nucleotide). Exemplary PAM sequences for RNA-guided nucleases, such as Cas9, are known to those of skill in the art and include, without limitation, NNG, NGN, NAG, NGA, NGG, NGAG and NGCG wherein N represents any nucleotide. In addition, Cas9 nucleases from different species have been described, e.g., S. thermophilus recognizes a PAM that comprises the sequence NGGNG, and Cas9 from S. aureus recognizes a PAM that comprises the sequence NNGRRT. In some embodiments, Cas9 from S. aureus recognizes a PAM that comprises the sequence NNNRRT. Additional PAM sequences are known in the art, including, but not limited to NNAGAAW and NAAR (see, e.g., Esvelt and Wang, Molecular Systems Biology, 9:641 (2013), the entire content of which is incorporated herein by reference). For example, the target site of an RNA-guided nuclease, such as, e.g., Cas9, may comprise a structure [Nz]-[PAM], where each N is, independently, any nucleotide, and z is an integer between 1 and 50. In some embodiments, z is at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, or at least 50. In some embodiments, z is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50. In some embodiments, Z is 20.

Treatment: As used herein, the term “treatment” (also “treat” or “treating”) refers to any administration of a therapy that partially or completely alleviates, ameliorates, eliminates, reverses, relieves, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder and/or condition and/or of a subject who exhibits only early signs of the disease, disorder, and/or condition. Alternatively, or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of a given disease, disorder, and/or condition.

Variant: As used herein, the term “variant” refers to a version of something, e.g., a gene sequence, that is different, in some way, from another version. To determine if something is a variant, a reference version is typically chosen and a variant is different relative to that reference version. In some embodiments, a variant can have the same or a different (e.g., increased or decreased) level of activity or functionality than a wild type sequence. For example, in some embodiments, a variant can have improved functionality as compared to a wild-type sequence if it is, e.g., codon-optimized to resist degradation, e.g., by an inhibitory nucleic acid, e.g., miRNA. Such a variant is referred to herein as a gain-of-function variant. In some embodiments, a variant has a reduction or elimination in activity or functionality or a change in activity that results in a negative outcome (e.g., increased electrical activity resulting in chronic depolarization that leads to cell death). Such a variant is referred to herein as a loss-of-function variant. For example, in some embodiments, a KCNQ4 gene sequence is a wild-type sequence, which encodes a functional protein and exists in a majority of members of species with genomes containing the KCNQ4 gene. In some such embodiments, a gain-of-function variant can be a gene sequence of KCNQ4 that contains one or more nucleotide differences relative to a wild-type KCNQ4 gene sequence. In some embodiments, a gain-of-function variant is a codon-optimized sequence which encodes a transcript or polypeptide that may have improved properties (e.g., less susceptibility to degradation, e.g., less susceptibility to miRNA mediated degradation) than its corresponding wild type(e.g., non-codon optimized) version. In some embodiments, a loss-of-function variant has one or more changes that result in a transcript or polypeptide that is defective in some way (e.g., decreased function, non-functioning) relative to the wild type transcript and/or polypeptide. For example, in some embodiments, a mutation in a KCNQ4 sequence results in a non-functional or otherwise defective KCNQ4 protein, which impairs or prevents function of a KCNQ4-containing potassium channel in ear outer hair cells. In some such embodiments, such loss-of-function variant KCNQ4-containing channels result in chronic depolarization of outer hair cells and, consequently, cell death.

### BRIEF DESCRIPTION OF THE DRAWING

FIG. 1 shows overall schematic of exons in a KCNQ4 gene and lists certain exemplary mutations known to result in hearing loss.

FIG. 2 shows an exemplary inhibitory RNA knockdown strategy using at least one construct, in accordance with an embodiment of the present disclosure.

FIG. 3 shows levels of expression of KCNQ4 in HEK293 cells with or without treatment with exemplary shRNA-mediated knockdown.

FIG. 4 shows exemplary shRNA and miRNA constructs for KCNQ4 knockdown.

FIG. 5 shows exemplary designs of eight miRNA targeting constructs for inhibiting KCNQ4 expression.

FIG. 6 depicts a schematic that displays exemplary miRNA constructs for choosing miRNAs targeting sequences expressed within a variety of compartments within the ear.

FIG. 7 shows a schematic of exemplary miR constructs and reporter system for KCNQ4 knockdown, in accordance with an embodiment of the present disclosure. In this reporter system, if an miRNA binds to KCNQ4-mScarlet mRNA, cleavage of the mRNA occurs and no detectable signal (mScarlet) is produced by the reporter, demonstrating effective KCNQ4 knockdown. By contrast, if an miRNA does not bind to KCNQ4-mScarlet mRNA, no cleavage occurs and a detectable signal (mScarlet) produced, demonstrating KCNQ4 expression (i.e., no knockdown).

FIG. 8 shows an exemplary miR mediated knockdown (miR1-155), using a luciferase reporter assay in accordance with an embodiment of the present disclosure. FIG. 8 discloses SEQ ID NOS 292-296, respectively, in order of appearance.

FIG. 9 shows an exemplary CRISPR-based construct and reporter system for KCNQ4 knockdown, in accordance with an embodiment of the present disclosure.

FIG. 10 shows levels of expression of KCNQ4 in HEK cells after CRISPR-mediated knockdown (N=3 biological replicates).

FIG. 11 shows results from an exemplary assay measuring levels of mRNA expression of KCNQ4 in HEK cells after shRNA-mediated knockdown (N=3 biological replicates per condition).

FIG. 12 shows results from an exemplary assay measuring levels of mRNA expression of KCNQ4 in HEK cells after shRNA-mediated knockdown (N=3 biological replicates per condition).

FIG. 13 shows an exemplary miR-based construct, reporter system and assay for KCNQ4 knockdown, in accordance with an embodiment of the present disclosure.

FIGS. 14A-14B show results obtained when exemplary scaffolds and targeting sequences were evaluated and level of KCNQ4 was assessed after evaluation.

FIG. 15 shows results obtained when exemplary scaffolds and targeting sequences were used and KCNQ4 levels were assessed.

FIG. 16 shows results obtained when exemplary scaffolds and targeting sequences were evaluated using an exemplary off-target reporter assay.

FIG. 17 shows results obtained when exemplary scaffolds and targeting sequences were evaluated using an exemplary passenger reporter assay.

FIG. 18 shows results obtained when exemplary scaffolds and targeting sequences were used and KCNQ4 levels were assessed.

FIG. 19 shows results obtained when exemplary scaffolds and targeting sequences were used and KCNQ4 levels were assessed.

FIG. 20 shows results obtained when exemplary scaffolds and targeting sequences were used and KCNQ4 levels were assessed.

FIG. 21 shows in vitro knockdown of KCNQ4 by exemplary scaffolds and targeting sequences described herein.

FIG. 22 shows an image depicting a western blot to assess in vitro knockdown of KCNQ4 using exemplary scaffolds and targeting sequences described herein.

FIG. 23 shows in vitro knockdown of KCNQ4 by exemplary scaffolds and targeting sequences described herein.

FIG. 24 shows an image depicting a western blot to assess in vitro knockdown of KCNQ4 using exemplary scaffolds and targeting sequences described herein.

FIG. 25 shows results obtained when exemplary scaffolds and targeting sequences were evaluated and level of KCNQ4 was assessed after evaluation.

FIG. 26 shows images of HEK cells transduced with an exemplary AAVAnc80-CAG.EGFP construct at three MOIs.

FIG. 27 shows results obtained when exemplary scaffolds and targeting sequences were used and KCNQ4 channel conductance levels were assessed.

FIG. 28 shows an image depicting a western blot to assess in vitro knockdown of KCNQ4 using exemplary scaffolds and targeting sequences described herein.

FIGS. 29A-29B is a schematic representation of an administration method as described herein. FIG. 29A includes an image of a delivery device as described herein. A delivery device as shown is intended for intracochlear administration of injected fluid through the round window membrane, with a stopper (green) to guide insertion depth. FIG. 29B includes an images showing an expected flow of injected fluid through scala tympani to scala vestibuli (via communication at the helicotrema at the cochlear apex) and then out of the cochlea through a vent placed in the stapes footplate of a delivery device within the oval window (Talei 2019, which is incorporated herein in its entirety by reference).

FIG. 30 depicts seven miRNA targeting constructs for inhibiting KCNQ4 expression that can be used in accordance with the present disclosure.

FIG. 31 depicts off-targets of seven miRNA targeting constructs for inhibiting KCNQ4 expression that can be used in accordance with the present disclosure.

FIG. 32 illustrates a perspective of a device for delivering fluid to an inner ear, according to aspects of the present disclosure.

FIG. 33 illustrates a sideview of a bent needle sub-assembly, according to aspects of the present disclosure.

FIG. 34 illustrates a perspective view of a device for delivering fluid to an inner ear, according to aspects of the present disclosure.

FIG. 35 illustrates a perspective view of a bent needle sub-assembly coupled to the distal end of a device, according to aspects of the present disclosure.

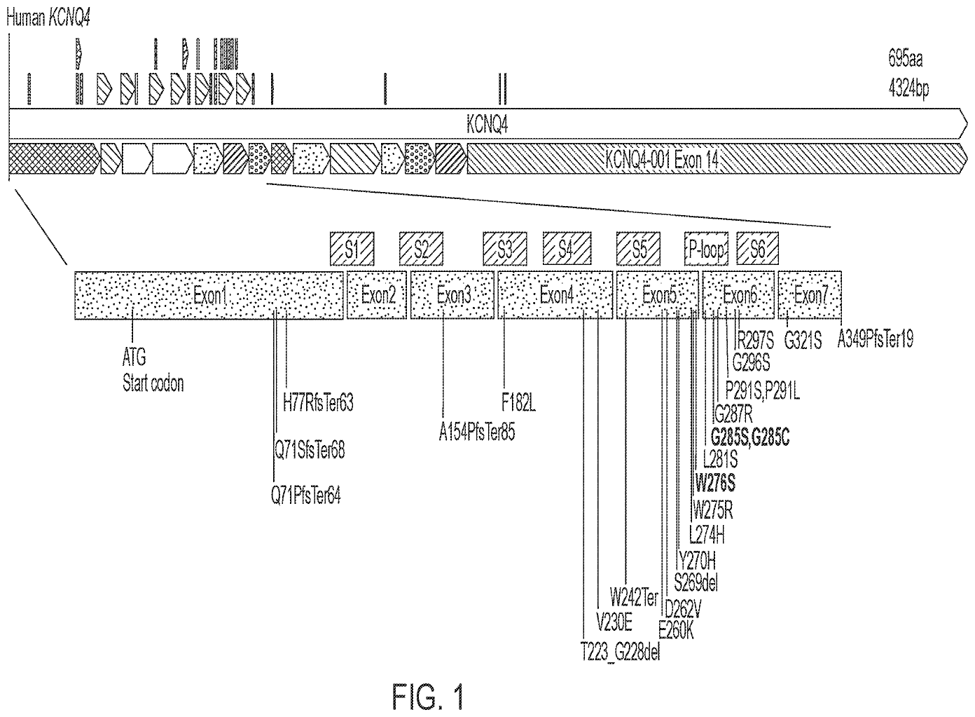

### DETAILED DESCRIPTION OF CERTAIN EMBODIMENTS

### 1. Hearing Loss

Generally, an ear can be described as including: an outer ear, middle ear, inner ear, hearing (acoustic) nerve, and auditory system (which processes sound as it travels from the ear to the brain). In addition to detecting sound, ears also help to maintain balance. Thus, in some embodiments, inner ear disorders can cause hearing loss, tinnitus, vertigo, imbalance, or combinations thereof.

Hearing loss can be a result of genetic factors, environmental factors, or a combination of genetic and environmental factors. About half of all people who have tinnitus-phantom noises in their auditory system (ringing, buzzing, chirping, humming, or beating)—also have an over-sensitivity to/reduced tolerance for certain sound frequency and volume ranges, known as hyperacusis (also spelled hyperacousis). A variety of nonsyndromic and syndromic-related hearing losses will be known to those of skill in the art (e.g., DFNB4, and Pendred syndrome, respectively). Environmental causes of hearing impairment or loss may include, e.g., certain medications, specific infections before or after birth, and/or exposure to loud noise over an extended period. In some embodiments, hearing loss can result from noise, ototoxic agents, presbyacusis, disease, infection or cancers that affect specific parts of an ear. In some embodiments, ischemic damage can cause hearing loss via pathophysiological mechanisms. In some embodiments, intrinsic abnormalities, like congenital mutations to genes that play an important role in cochlear anatomy or physiology, or genetic or anatomical changes in supporting and/or hair cells can be responsible for or contribute to hearing loss.

Hearing loss and/or deafness is one of the most common human sensory deficits, and can occur for many reasons. In some embodiments, a subject may be born with hearing loss or without hearing, while others may lose hearing slowly over time. Approximately 36 million American adults report some degree of hearing loss, and one in three people older than 60 and half of those older than 85 experience hearing loss. Approximately 1.5 in 1,000 children are born with profound hearing loss, and another two to three per 1,000 children are born with partial hearing loss (Smith et al., 2005, Lancet 365:879-890, which is incorporated in its entirety herein by reference). More than half of these cases are attributed to a genetic basis (Di Domenico, et al., 2011, J. Cell. Physiol. 226:2494-2499, which is incorporated in its entirety herein by reference).

Treatments for hearing loss currently consist of hearing amplification for mild to severe losses and cochlear implantation for severe to profound losses (Kral and O'Donoghue, 2010, N. Engl. J. Med. 363:1438-1450, which is incorporated in its entirety herein by reference). Recent research in this arena has focused on cochlear hair cell regeneration, applicable to the most common forms of hearing loss, including presbycusis, noise damage, infection, and ototoxicity. There remains a need for effective treatments, such as gene therapy, which can repair and/or mitigate a source of a hearing problem.

In some embodiments, nonsyndromic hearing loss and/or deafness is not associated with other signs and symptoms. In some embodiments, syndromic hearing loss and/or deafness occurs in conjunction with abnormalities in other body parts. Approximately 70 percent to 80 percent of genetic hearing loss and/or deafness cases are nonsyndromic; remaining cases are often caused by specific genetic syndromes. Nonsyndromic deafness and/or hearing loss can have different patterns of inheritance, and can occur at any age. Types of nonsyndromic deafness and/or hearing loss are generally named according to their inheritance patterns. For example, autosomal dominant forms are designated DFNA, autosomal recessive forms are DFNB, and X-linked forms are DFN. Each type is also numbered in the order in which it was first described. For example, DFNA1 was the first described autosomal dominant type of nonsyndromic deafness.

Between 75 percent and 80 percent of genetically causative hearing loss and/or deafness cases are inherited in an autosomal recessive pattern, which means both copies of a gene in each cell have mutations. Usually, each parent of an individual with autosomal recessive hearing loss and/or deafness is a carrier of one copy of the mutated gene, but is not affected by this form of hearing loss. Another 20 percent to 25 percent of nonsyndromic hearing loss and/or deafness cases are autosomal dominant, which means one copy of the altered gene in each cell is sufficient to result in deafness and/or hearing loss. People with autosomal dominant deafness and/or hearing loss most often inherit an altered copy of the gene from a parent who is deaf and/or has hearing loss. Between one to two percent of cases of deafness and/or hearing loss show an X-linked pattern of inheritance, which means a mutated gene responsible for a condition is located on an X chromosome (one of the two sex chromosomes). Males with X-linked nonsyndromic hearing loss and/or deafness tend to develop more severe hearing loss earlier in life than females who inherit a copy of the same gene mutation. A characteristic of X-linked inheritance is that fathers cannot pass X-linked traits to their sons.

Mitochondrial nonsyndromic deafness, which results from changes to mitochondrial DNA, occurs in less than one percent of cases in the United States. Altered mitochondrial DNA is passed from a mother to all of her sons and daughters. This type of deafness is not inherited from fathers. The causes of syndromic and nonsyndromic deafness and/or hearing loss are complex. Researchers have identified more than 30 genes that, when altered, are associated with syndromic and/or nonsyndromic deafness and/or hearing loss; however, some of these genes have not been fully characterized. Different mutations in a given gene can be associated with different types of deafness and/or hearing loss, and some genes are associated with both syndromic and nonsyndromic deafness and/or hearing loss.

In some embodiments, deafness and/or hearing loss can be conductive (arising from the ear canal or middle ear), sensorineural (arising from the inner ear or auditory nerve), or mixed. In some embodiments, nonsyndromic deafness and/or hearing loss is associated with permanent hearing loss caused by damage to inner ear structures (sensorineural deafness). In some embodiments, sensorineural hearing loss can be due to poor hair cell function. In some embodiments, sensorineural hearing impairments involve the eighth cranial nerve (the vestibulocochlear nerve) or auditory brain regions. In some such embodiments, only auditory centers of a brain are affected. In such a situation, cortical deafness may occur, where sounds may be heard at normal thresholds, but quality of sound perceived is so poor that speech cannot be understood.

Hearing loss that results from middle ear changes is called conductive hearing loss. Some forms of nonsyndromic deafness and/or hearing loss involve changes in both inner and middle ear regions, called mixed hearing loss. Hearing loss and/or deafness that is present before a child learns to speak can be classified as prelingual or congenital. Hearing loss and/or deafness that occurs after development of speech can be classified as postlingual. Most autosomal recessive loci related to syndromic or nonsyndromic hearing loss cause prelingual severe-to-profound hearing loss.

As is known to those of skill in the art, hair cells are sensory receptors for both auditory and vestibular systems of vertebrate ears. Hair cells detect movement in their environments and, in mammals, hair cells are located within the cochlea of the ear, in the organ of Corti. Mammalian ears are known to have two types of hair cells—inner hair cells and outer hair cells. In some embodiments, outer hair cells amplify low level sound frequencies, either through mechanical movement of hair cell bundles or electrically-driven movement of hair cell soma. In some embodiments, inner hair cells transform vibrations in cochlear fluid into electrical signals that the auditory nerve transmits to the brain. In some embodiments, hair cells may be abnormal at birth, or damaged during the lifetime of an individual. In some embodiments, outer hair cells may be able to regenerate. In some embodiments, inner hair cells are not capable of regeneration after illness or injury. In some embodiments, sensorineural hearing loss is due to abnormalities in hair cells.

As is known to those of skill in the art, hair cells do not occur in isolation, and their function is supported by a wide variety of cells which can collectively be referred to as supporting cells. Supporting cells may fulfill numerous functions, and include a number of cell types, including but not limited to Hensen's cells, Deiters' cells, pillar cells, Claudius cells, inner phalangeal cells, and border cells. In some embodiments, sensorineural hearing loss is due to abnormalities in supporting cells. In some embodiments, supporting cells may be abnormal at birth, or damaged during the lifetime of an individual. In some embodiments, supporting cells may be able to regenerate. In some embodiments, certain supporting cells may not be capable of regeneration.

### 2. KCNQ4 and Hearing Loss

Typically, a human KCNQ4 gene has 4324 nucleic acid bases and encodes a 695 amino acid protein, with a predicted mass of approximately 77 kDa. A human KCNQ4 gene has 14 exons and located on chromosome 1. A KCNQ4 gene encodes a Kv7.4, a voltage-gated potassium channel subunit that forms a homotetrameric potassium channel. In other words, four KCNQ4 protein subunits form a single, voltage-gated potassium channel (Naito et al., 2013, which is incorporated herein by reference in its entirety). In some embodiments, a KCNQ4 protein (Kv7.4) may be part of a heteromeric channel, i.e., a heterotetrameric potassium channel comprising Kv7.4 and other KCNQ proteins, e.g., KCNQ3.

In some embodiments, one or more mutations in a KCNQ4 gene product may be associated with hearing loss. In some embodiments, distortion product optoacoustic emissions (DPOAEs) are absent in individuals affected by KCNQ4-mediated hearing loss.

Voltage-gated potassium channel subunit Kv7.4 is a protein encoded by aKCNQ4 gene and is normally most highly expressed in outer hair cells of the ear. As is known to those of skill in the art, OHCs are non-regenerative cells. Kv7.4 channels help maintain resting potential in ear hair cells. For example, Kv7.4 is expressed in bases of hair cells that help maintain hair cell resting potential (Kharkovets et al., 2006, which is incorporated in its entirety herein by reference) and in some embodiments, is expressed at approximately 8-fold higher in OHCs than IHCs.

Without being bound by any particular theory, the present disclosure contemplates that, either in addition or as an alternative to gene therapy that treats OHCs, gene therapy may be used to treat IHCs. KCNQ4 is typically expressed at low levels in IHCs (e.g., relative to OHCs) and, in some embodiments, gene therapy that impacts expression of KCNQ4 in IHCs may improve function of K+ channels. In some such embodiments, when gene therapy is used to treat IHCs instead of or in addition to OHCs in a subject in need thereof, channel conduction and/or hearing may improve.

In some embodiments, defects or changes in ion channels are associated with deafness. For example, in some embodiments, a change in a gene product of an ion channel, e.g., a Kv7.4 channel, may impact its function. In some embodiments, for example, one or more mutations in a KCNQ4 gene product can result in a non-functional or less functional ion channels as compared to ion channels comprises of subunits encoded by genes without one or more mutations. In some such embodiments, where one or more variations exists in KCNQ4 (e.g., relative to a wild-type sequence), for example, a resultant loss-of-function Kv7.4 protein variant can result in a non-functional or less functional channel. For example, in some embodiments, a loss-of-function Kv7.4 variant is or is part of an ion channel that antagonizes potassium currents. In some such embodiments, OHCs are chronically depolarized and eventually die (Jung et al., 2018, which is incorporated in its entirety herein by reference). In some embodiments, changes in one or more gene products of KCNQ4 is/are associated with hearing loss.

In some embodiments, KCNQ4-mediated hearing loss is DFNA2. DFNA2 is nonsyndromic hearing loss inherited as an autosomal dominant mutation in a genomic sequence of KCNQ4 (which, in turn, impacts function of Kv7.4 in hair cells). DFNA2 nonsyndromic hearing loss, in some embodiments, manifests as sensorineural post-lingual hearing impairment that is progressive and symmetric; generally, no vestibular impairment is present. In some such embodiments, hearing loss is symmetric, predominantly high-frequency sensorineural hearing loss (SNHL). In some such embodiments, hearing loss is progressive or eventually progresses across all frequencies.

Without wishing to be bound by any particular theory, in KCNQ4-related hearing loss, at younger ages, hearing loss tends to be mild for low frequency sounds and moderate for high frequency sounds. At older ages, hearing loss tends to moderate for low frequency sounds and severe to profound for high frequency sounds. Hearing loss tends to be present at high frequency sounds at all ages, likely present from birth. In some embodiments, patients with a KCNQ4 mutation experience hearing loss that requires a hearing aid between approximately ten to forty years of age, and experience severe-to-profound loss across all hearing frequencies by approximately seventy years of age (see, e.g., Table 1 for an exemplary set of range characterizations of hearing threshold vs severity of hearing loss)

TABLE 1

| Severity | Threshold (dB) | |
|---|---|---|
| Mild | 26-40 | dB |
| Moderate | 41-55 | dB |
| Moderately-severe | 56-70 | dB |
| Severe | 71-90 | dB |
| Profound | 90 | dB |

Without being bound by any particular theory, the present disclosure contemplates technologies including gene therapy may be beneficial in one or more instances of a loss-of-function KCNQ4 variant. For example, in some embodiments, gene therapy includes administering a gain-of-function KCNQ4 variant (e.g., wild type, e.g., gain-of-function KCNQ4) that restores function, e.g., Kv7.4 channel function. In some embodiments, a gain-of-function KCNQ4 gene product (e.g., a wild type gene product, e.g., a codon-optimized gene product) is administered to a subject in need thereof.

In some embodiments, gene therapy includes suppressing one or more gene products associated with a loss-of-function KCNQ4 variant In some such embodiments, suppression of a loss-of-function KCNQ4 variant may help to restore or prevent hearing loss. For example, without being bound by any particular theory, it is contemplated that, in some embodiments, a loss-of-function KCNQ4 variant gene product encodes a loss-of-function Kv7.4 variant. In some such embodiments, it is contemplated that such a loss-of-function Kv7.4 variant needs to be suppressed. In some embodiments, suppression alleviates toxicity or damage caused by a buildup of loss-of-function Kv7.4 variant protein. In some embodiments, a loss-of-function KCNQ4 variant gene product (e.g., Kv7.4 variant protein) is suppressed using gene therapy.

In some embodiments, gene therapy is administered to suppress a loss-of-function KCNQ4 variant and/or to express a gain-of-function KCNQ4 gene product (e.g., a wild type gene product, e.g., a codon optimized gene product). In some such embodiments, suppression and/or replacement of one or more KCNQ4 gene products mitigates, attenuates, or restores hearing loss in a subject. For example, the present disclosure recognizes that, in some embodiments, suppression of a loss-of-function KCNQ4 gene product variant (e.g., mRNA, e.g., protein) is desirable. In some such embodiments, suppression of a loss-of-function KCNQ4 gene product variant may occur alone, concomitant with, or subsequent to expression of a gain-of-function KCNQ4 gene product (e.g., functional Kv7.4 protein, e.g., functional ion channels that do not result in chronic depolarization and cell damage or death).

In some embodiments, suppression and/or replacement is accomplished using a single construct, or more than one construct (e.g., one construct comprising components to achieve suppression of a loss-of-function KCNQ4 variant gene product and another construct comprising components to achieve expression of a gain-of-function KCNQ4 gene product).

### 3. Compositions

Among other things, the present disclosure provides compositions. In some embodiments, a composition comprises a construct as described herein. In some embodiments, a composition comprises one or more constructs as described herein. In some embodiments, a composition comprises a plurality of constructs as described herein. In some embodiments, when more than one construct is included in the composition, the constructs are different from one another.

In some embodiments, a composition comprises a polynucleotide encoding a KCNQ4 protein or characteristic portion thereof. In some embodiments, a composition comprises a polynucleotide encoding an inhibitory molecule, e.g., an miRNA, etc. In some embodiments, a composition comprises at least one polynucleotide encoding a KCNQ4 protein or characteristic portion thereof and at least one polynucleotide encoding an inhibitory molecule, e.g., an miRNA.

In some embodiments, a composition comprises an AAV particle as described herein. In some embodiments, a composition comprises one or more AAV particles as described herein. In some embodiments, a composition comprises a plurality of AAV particles. In come embodiments, when more than one type of AAV particle is included in a composition, the more than one type of AAV particles are each different types of particles.

In some embodiments, a composition comprises a cell.

In some embodiments, a composition is or comprises a pharmaceutical composition.

a. KCNQ4 Polynucleotides

Among other things, the present disclosure provides polynucleotides, e.g., polynucleotides comprising a KCNQ gene or characteristic portion thereof, as well as compositions comprising inhibitory molecules, e.g., inhibitory to target site of KCNQ4 genes or characteristics thereof, e.g., miRNA, etc. The present disclosure also provides methods utilizing such polynucleotides and/or compositions.

In some embodiments, a polynucleotide of the present disclosure may be or comprise DNA or RNA. In some embodiments, DNA can be genomic DNA or cDNA. In some embodiments, RNA can be an mRNA, an miRNA, an shRNA/siRNA, a gRNA, etc.

In some embodiments, a polynucleotide comprises exons and/or introns of a KCNQ4 gene.

In some embodiments, a gene product is expressed from a polynucleotide comprising a KCNQ4 gene or characteristic portion thereof. In some embodiments, expression of such a polynucleotide can utilize one or more control elements (e.g., promoters, enhancers, splice sites, polyadenylation sites, translation initiation sites, etc.). Thus, in some embodiments, a polynucleotide provided herein can comprise one or more control elements.

In some embodiments, a KCNQ4 gene is a mammalian KCNQ4 gene. In some embodiments, a KCNQ4 gene is a murine KCNQ4 gene. An exemplary murine KCNQ4 cDNA sequence is or includes the sequence of SEQ ID NO: 91. In some embodiments, a KCNQ4 gene is a primate KCNQ4 gene. In some embodiments, a KCNQ4 gene is a human KCNQ4 gene. An exemplary human KCNQ4 cDNA sequence is or includes the sequence of SEQ ID NO: 2. An exemplary human KCNQ4 cDNA sequence is or includes the sequence of SEQ ID NO: 90. An exemplary human KCNQ4 genomic DNA sequence can be found in SEQ ID NO: 5. Exemplary human KCNQ4 cDNA sequences including untranslated regions is or includes the sequence of SEQ ID NOs: 6, 7, or 8. An exemplary human KCNQ4 RNA sequence can be found in, e.g., SEQ ID NO: 1. Exemplary human codon-optimized KCNA4 sequences (e.g., optimized to resist gRNA binding and/or miRNA degradation) can be found in, e.g., SEQ ID NOs: 9 or 10.

For example, in some embodiments, the present disclosure describes exemplary constructs that have been codon-optimized (e.g., Exemplary pITR-CMV.hKCNQ4codop_v2.mScarlet, SEQ ID NO: 255) to resist microRNA. In some embodiments, the present disclosure recognizes that one challenge of exogenously providing a polynucleotide that encodes a functional (e.g., wild type, e.g., gain-of-function) KCNQ4 gene product is that it may, in some embodiments, be vulnerable to microRNA-mediated (e.g., exogenously provided and/or endogenous miRNAs) degradation. In some such embodiments, the present disclosure recognizes that codon optimization, which may change a polynucleotide sequence without materially altering the polypeptide sequence of a KCNQ4 gene product, may be more resistant to microRNA-mediated degradation as compared to a non-codon optimized (i.e., wild-type) KCNQ4 gene sequence. In some embodiments, a construct comprising a codon-optimized KCNQ4 polynucleotide can be used in conjunction with a construct comprising an miRNA. In some embodiments, such miRNA can be used to knock-down (or suppress) a loss-of-function KCNQ4 variant.

As another example, in some embodiments, the present disclosure describes exemplary constructs that can for delivery of a gRNA to be used in conjunction with a CRISPR/Cas9-mediated genome editing strategy as described herein. In some embodiments, such exemplary constructs comprise a gRNA that targets a SaCas9 enzyme to an appropriate genomic location. In some embodiments, such exemplary constructs comprise a gRNA that targets a SaCas9 enzyme to an appropriate genomic location in addition to a KCNQ4 construct that has been engineered to resist SaCas9-mediated gene silencing (e.g., Exemplary Construct pITR-CMV.hKCNQ4codop.U6-hsammu386Fw sequence (SEQ ID NO: 269) or Exemplary pITR-CMV.hKCNQ4codop.U6-hsa408Rev sequence (SEQ ID NOs: 270 or 273).

The present disclosure recognizes that one challenge of exogenously providing a polynucleotide that encodes a functional (e.g., wild type, e.g., gain-of-function) KCNQ4 gene product is that it may, in some embodiments, be vulnerable to microRNA-mediated degradation (exogenously provided and/or endogenous miRNAs) or gRNA interference (e.g., via gRNA binding). In some such embodiments, the present disclosure recognizes that codon optimization, which changes a polynucleotide sequence without materially altering its resultant polypeptide sequence, of, e.g., a KCNQ4 gene product, may be more resistant to microRNA-mediated degradation or to gRNA binding, as compared to a non-codon optimized (i.e., wild-type) KCNQ4 gene sequence. In some embodiments, a construct comprising a codon-optimized KCNQ4 polynucleotide can be used in conjunction with a construct comprising an miRNA, which miRNA can be used to knock-down (suppress) a loss-of-function KCNQ4 variant. Exemplary codon-optimized sequences that resist miRNA-mediated degradation or gRNA binding may be or comprise SEQ ID NO:s 9 or 10 or portions thereof, respectively.

Changes in a wild-type sequence of a KCNQ4 gene can be or comprise missense or nonsense mutations. In some such embodiments, a resultant Kv7.4 protein is a loss-of-function variant (e.g., a protein that antagonizes normal channel function). In some such embodiments, changes in a wild-type sequence of KCNQ4 can result in hearing loss or increase risk of hearing loss in progeny of a subject that has at least one change in one copy of a KCNQ4 gene.

KCNQ4-mediated hearing loss is transmitted in an autosomal dominant manner; that is, a mutation in one copy of KCNQ4 can result in hearing loss. Many allelic variants in KCNQ4 are known and at least thirty different loss-of-function KCNQ4 mutations have been identified, thus far, localized to various different genomic regions. In some embodiments, a change in a wild-type sequence is a change in an exonic sequence. For example, the three most frequent missense mutations described in the DVD (Deafness Variation Database) are F182L (exon 4), V672M and S680F (exon 14). In some embodiments, a change in a wild-type sequence is a change in an intronic sequence. For example, as is known to those of skill in the art, in some embodiments, an intronic (splice acceptor) mutation causes DFNA2 (c.1044-1051del) or A349PfsX19.

The present disclosure includes technologies that may, in some embodiments, targeting a KCNQ4 gene product, e.g., a KCNQ4 transcript, e.g., a KCNQ4 mRNA (SEQ ID NO:4). In some embodiments, an inhibitory nucleic acid molecule or genome editing system targets nucleotides of SEQ ID NOs: 1-10 and/or 25-30 and/or 90-91, or a portion thereof. In some embodiments, an inhibitory nucleic acid molecule or genome editing system comprises (i) a nucleotide sequence that is at least 85%, 90%, 91%, 92%, 93%, 94%, 95%, 96%, 97%, 98%, 99%, or 100% identical to any one of SEQ ID NOs:42-70 or SEQ ID NOs: 96-97 (or a portion thereof) and/or (ii) a nucleotide sequence that is complementary to a nucleotide sequence that is at least 90%, 91%, 92%, 93%, 94%, 95%, 96%, 97%, 98%, 99%, or 100% identical to any one of SEQ ID NOs:1-10 and/or 25-30 and/or 90-91 (or a portion thereof).

The amino acid and nucleotide sequences of human KCNQ4 are known in the art and can be found in publicly available databases, for example, the National Center for Biotechnology Information (NCBI) Reference Sequence (RefSeq) database, where they are listed under RefSeq accession numbers NP_004691 (current accession.version number NP_004691.2) and NM_004700 (current accession.version number NM_004700.4), respectively (where “amino acid sequence” refers to the sequence of the KCNQ4 polypeptide and “nucleotide sequence” in this context refers to the KCNQ4 mRNA sequence as represented in genomic DNA, it being understood that the actual mRNA nucleotide sequence contains U rather than T).

The human KCNQ4 gene has been assigned NCBI Gene ID: 9132, and the genomic KCNQ4 sequence has RefSeq accession number NG_008139 (current accession.version number NG_008139.3). The nucleotide sequence of human KCNQ4 mRNA is set forth as SEQ ID NO: 4.

In some embodiments, a KCNQ4 nucleic acid sequence is a codon optimized sequence. In some embodiments, the codon optimized sequence is approximately 70, 75, 80, 85, 90, 95, 96, 97, 98, 99, 99.5 or 99.9% similar to the wild-type KCNQ4 nucleic acid sequence or any known functional variant thereof, which variant is capable of generating a functional gene product.

The present disclosure recognizes that certain changes to a polynucleotide sequence will not impact its expression or a protein encoded by said polynucleotide. For example, in some embodiments, a polynucleotide comprises a KCNQ4 gene having one or more silent mutations. In some such embodiments, the disclosure provides a polynucleotide that comprises a KCNQ4 gene having one or more silent mutations, e.g., a KCNQ4 gene having a sequence different from SEQ ID NOs: 1-10 and/or 25-30 and/or 90-91, but encoding the same amino acid sequence as a wild-type or gain-of-function KCNQ4 gene.

In some embodiments, the disclosure provides a polynucleotide that comprises an KCNQ4 gene or gene product having a sequence different from any of SEQ ID NOs: 1-10 and/or 25-30 and/or 90-91 that encodes an amino acid sequence including one or more mutations (e.g., a different amino acid sequence when compared to that produced from a functional, e.g., wild type, e.g., gain-of-function (e.g., codon-optimized) KCNQ4 gene), where the one or more mutations are conservative amino acid substitutions.

One skilled in the art would appreciate that a change (e.g., substitution, addition, deletion, etc.) of amino acids that are not conserved between the same protein from different species is less likely to have an effect on the function of a protein and therefore, these amino acids should be selected for mutation. Amino acids that are conserved between the same protein from different species should not be changed (e.g., deleted, added, substituted, etc.), as these mutations are more likely to result in a change in function of a protein.

In some embodiments, the disclosure provides a polynucleotide that comprises a KCNQ4 gene having a sequence different from SEQ ID NOs: 1-10 and/or 25-30 and/or 90-91 that encodes an amino acid sequence including one or more mutations (e.g., a different amino acid sequence when compared to that produced from a functional (e.g., wild-type, e.g., gain-of-function, e.g., codon-optimized) KCNQ4 gene), where the one or more mutations are not within a characteristic portion of a KCNQ4 gene or encoded Kv7.4 protein.

In some embodiments, a polynucleotide in accordance with the present disclosure comprises a KCNQ4 gene or gene product that is at least 85%, at least 90%, at least 95%, at least 98%, or at least 99% identical to a sequence of SEQ ID NOs: 1-10 and/or 25-30 and/or 90-91.

In some embodiments, a polynucleotide in accordance with the present disclosure comprises KCNQ4 sequence that is identical to the sequence of SEQ ID NOs: 1-10 and/or 25-30 and/or 90-91. As will be understood to those of skill in the art, sequences disclosed herein can be optimized or further (e.g., codon optimized) to achieve increased or optimal expression in an animal, e.g., a mammal, e.g., a human. For example, in some embodiments, a polynucleotide of the present disclosure comprises a sequence encoding KCNQ4, which sequence is codon optimized to prevent, e.g., gRNA or miRNA binding, etc.

By way of non-limiting example, a KCNQ4 polynucleotide in accordance with the present disclosure may be or comprise one or more of the following:

EXEMPLARY HUMAN KCNQ4 mRNA SEQUENCE
(SEQ ID NO: 1)
GACAUGUGAGCGGCGCGCGCCGGUGGCAGGUGGAAAGGCGAGCGGCAUGGAGCGCGUAAUAAGA
GAGUUGGAGUCGGAAAGAGCAGCCCCAGUCGCCGGGGAAGCGGGAGGUCAGUGCGGGCUCCGGC
GGCCCCCAGGCUCCGAGCGCCCGCCCGCGGCCCCGGCCCGGCCCCUAGCCCCCGCCGCCCGCGC
CCGCCCCGGGUCGCCCCUCUGGCCCCGGGUCCGAGCCAUGCGUCUCUGAGCGCCCCGAGCGCGC
CCCCGCCCCGGACCGUGCCCGGGCCCCGGCGCCCCCAGCCCGGCGCCGCCCAUGGCCGAGGCCC
CCCCGCGCCGCCUCGGCCUGGGUCCCCCGCCCGGGGACGCCCCCCGCGCGGAGCUAGUGGCGCU
CACGGCCGUGCAGAGCGAACAGGGCGAGGCGGGCGGGGGCGGCUCCCCGCGCCGCCUCGGCCUC
CUGGGCAGCCCCCUGCCGCCGGGCGCGCCCCUCCCUGGGCCGGGCUCCGGCUCGGGCUCCGCCU
GCGGCCAGCGCUCCUCGGCCGCGCACAAGCGCUACCGCCGCCUGCAGAACUGGGUCUACAACGU
GCUGGAGCGGCCCCGCGGCUGGGCCUUCGUCUACCACGUCUUCAUAUUUUUGCUGGUCUUCAGC
UGCCUGGUGCUGUCUGUGCUGUCCACUAUCCAGGAGCACCAGGAACUUGCCAACGAGUGUCUCC
UCAUCUUGGAAUUCGUGAUGAUCGUGGUUUUCGGCUUGGAGUACAUCGUCCGGGUCUGGUCCGC
CGGAUGCUGCUGCCGCUACCGAGGAUGGCAGGGUCGCUUCCGCUUUGCCAGAAAGCCCUUCUGU
GUCAUCGACUUCAUCGUGUUCGUGGCCUCGGUGGCCGUCAUCGCCGCGGGUACCCAGGGCAACA
UCUUCGCCACGUCCGCGCUGCGCAGCAUGCGCUUCCUGCAGAUCCUGCGCAUGGUGCGCAUGGA
CCGCCGCGGCGGCACCUGGAAGCUGCUGGGCUCAGUGGUCUACGCGCAUAGCAAGGAGCUGAUC
ACCGCCUGGUACAUCGGGUUCCUGGUGCUCAUCUUCGCCUCCUUCCUGGUCUACCUGGCUGAGA
AGGACGCCAACUCCGACUUCUCCUCCUACGCCGACUCGCUCUGGUGGGGGACGAUUACAUUGAC
AACCAUCGGCUAUGGUGACAAGACACCGCACACAUGGCUGGGCAGGGUCCUGGCUGCUGGCUUC
GCCUUACUGGGCAUCUCUUUCUUUGCCCUGCCUGCCGGCAUCCUAGGCUCCGGCUUUGCCCUGA
AGGUCCAGGAGCAGCACCGGCAGAAGCACUUCGAGAAGCGGAGGAUGCCGGCAGCCAACCUCAU
CCAGGCUGCCUGGCGCCUGUACUCCACCGAUAUGAGCCGGGCCUACCUGACAGCCACCUGGUAC
UACUAUGACAGUAUCCUCCCAUCCUUCAGAGAGCUGGCCCUCUUGUUUGAGCACGUGCAACGGG
CCCGCAAUGGGGGCCUACGGCCCCUGGAGGUGCGGCGGGCGCCGGUACCCGACGGAGCACCCUC
CCGUUACCCGCCCGUUGCCACCUGCCACCGGCCGGGCAGCACCUCCUUCUGCCCUGGGGAAAGC
AGCCGGAUGGGCAUCAAAGACCGCAUCCGCAUGGGCAGCUCCCAGCGGCGGACGGGUCCUUCCA
AGCAGCAUCUGGCACCUCCAACAAUGCCCACCUCCCCAAGCAGCGAGCAGGUGGGUGAGGCCAC
CAGCCCCACCAAGGUGCAAAAGAGCUGGAGCUUCAAUGACCGCACCCGCUUCCGGGCAUCUCUG
AGACUCAAACCCCGCACCUCUGCUGAGGAUGCCCCCUCAGAGGAAGUAGCAGAGGAGAAGAGCU
ACCAGUGUGAGCUCACGGUGGACGACAUCAUGCCUGCUGUGAAGACAGUCAUCCGCUCCAUCAG
GAUUCUCAAGUUCCUGGUGGCCAAAAGGAAAUUCAAGGAGACACUGCGACCGUACGACGUGAAG
GACGUCAUUGAGCAGUACUCAGCAGGCCACCUGGACAUGCUGGGCCGGAUCAAGAGCCUGCAAA
CUCGGGUGGACCAAAUUGUGGGUCGGGGGCCCGGGGACAGGAAGGCCCGGGAGAAGGGCGACAA
GGGGCCCUCCGACGCGGAGGUGGUGGAUGAAAUCAGCAUGAUGGGACGCGUGGUCAAGGUGGAG
AAGCAGGUGCAGUCCAUCGAGCACAAGCUGGACCUGCUGUUGGGCUUCUAUUCGCGCUGCCUGC
GCUCUGGCACCUCGGCCAGCCUGGGCGCCGUGCAAGUGCCGCUGUUCGACCCCGACAUCACCUC
CGACUACCACAGCCCUGUGGACCACGAGGACAUCUCCGUCUCCGCACAGACGCUCAGCAUCUCC
CGCUCGGUCAGCACCAACAUGGACUGAGGGACUUCUCAGAGGCAGGGCAGCACACGGCCAGCCC
CGCGGCCUGGCGCUCCGACUGCCCUCUGAGGCCUCCGGACUCCUCUCGUACUUGAACUCACUCC
CUCACGGGGAGAGAGACCACACGCAGUAUUGAGCUGCCUGAGUGGGCGUGGUACCUGCUGUGGG
UGCCAGCGCCCCUUCCCCACCUCAGGAGCGUGAGAUGCCAGGUCGCACAGAGGGCAGCAGCAGC
GGCCGUCCCGCGGCCUCUGGGCCCCCCAGUGCCCUGCCCACUCCAUCAAGGCCCUAUGUGGCCC
ACCUGGCAGGGGCACAGCCCCGGGAGUGGGAGCGGGCGCUGGGGCCCUGGGCCCUGACCCAGCU
UCCAGCUAUGCAAGGUGAGGUCUCUGGCCCACCCUUCGGACACAGCAGGGAAGCCCUCCCGCCA
AGUCCCCGCCCCACUUGGGGGUGGGCCAAGGUGCCCCCACAGGUACCCACAAAGCACAGGACCC
UGCCACAAGGCAGGUGGACACCAUAUAUGCAAACCAUGUUAAAUAUGCAACUUUGGGGACCCCC
AUGGGGUCUCUCUGUCCCUCCCCCAUUGGGAGCUGGGCCCCCAGCAGUAGCUGGUCUCAGGCUG
CUUGGCCACCACCCUGUCCCUAUUCUUUGGCUUAUCACUCCUUCCCCUCCCAGCAUGGGGCCUG
UUUCUCCCCUGCCCUCUCCUAAGGGCAAUGCCUGGGCCUUUCUUCCCAUUUGCAAGUGUCAGCU
CCCAGGGGCUCCCUCCUCCUGCUGGGUGGCCACUCCCCUCCUUGGCCCUCCAGACACCACUCAU
AGUCAGCACAGGUUUCUGUAUCCUCCCCAAAACUCCCAGACAGUGCUUCGUGGACGAUCGCACA
AACAUAGCCUUUUAGUUUCUCCAGACAGGAAGAAAGCCUCUCACACUUAAACAUGCAAUGACGU
GACACACUUGGAGACAUGAGUGCAGAGCCACUCAGCCGCUCCUGGGCCUCUGCAGCAGAUGCCA
GUGGACUGGCCUUGCAGGGUGACGACCACUAAGAGGAAGACCCCCAACUCCAUCUGAGCAGGAG
AAGGAGCUUUGAAGUAACCCGAGAGCUCUCCAGGCCCCACCCAGACCUUUACCCGCUCCCCUUC
UUCAAGAAGAUCUCCUCCUCUCUGGUCCAGGAGCCCUAACCCACUGCCUCUGCCUGUCCCCAAG
GGCCCGCCUCCGUGUCUCCACAGCACAACUCGGGCCCAGGCCUGACACCACUGGAGAGACCCCA
GGCCCACUUCUAGCCAGGCCUGUGCCUUCCUAGUCACUCUAACUCCCAGAGAGAAUAAGAAUGC
AUGUAAUAGCUAUACCAACCGCGCAUCCGGCUUUCACAUGCACUGUCUCCCCUCCCUCCACACC
CCACUUCUUCACUUCAAUUGGCAGCGCCACAUCCAGGCGUCAGCCCCCAUUCACUCCAGGAACA
CUUUCUUAUCCCCACCCCUUUGCUCCUCUUCUGCAAAGCCAAUGCAGGUGGCAGGAAGGUGAGG
GGUAGUGGACCAAUGGCAACCCUCUGUGGGAACAAGGGGCCGAGGCCACGCUGCCUGCAUCUCG
UGCUGGGGACCUGCAUGCGCCAGCACCAGGGCUUGGACUGGAUCUUACUCAGUCCAUGGUGCCC
AGCCUCUGCCCCAACAUGCCCUCUGCAUGUGACCGUCAUGCCCUGGAUGGAGCCACUCCUGGCU
CACCCCACCUGCACUGCACUGUCCCCAGAGAGCCACCCCUCCACCCACUCAGAGACAGCUGUGG
AGAGGGCCAGGAGAAUGGGAUUACCCUAUGACCAAGGAGACAUGGGAAGAAGCCCUCCUUCCUU
CCACGAUCGAGGUUCCGCCAUCAACUCGGUUCUCGGAUAUGCAAGUACCUCACUUUGUUAACUU
AUUAACUUAUUGGUUUCAUUAAAGUUUUCAAGAGGA; (from RefSeq accession number
NM_004700.4 with Treplaced by U; AUG initiation codon in underlined
bold italics, starting at position 308).
EXEMPLARY HUMAN KCNQ4 ISOFORM A cDNA CODING
SEQUENCE
(SEQ ID NO: 2)
ATGGCCGAGGCCCCCCCGCGCCGCCTCGGCCTGGGTCCCCCGCCCGGGGACGCCCCCCGCGCGG
AGCTAGTGGCGCTCACGGCCGTGCAGAGCGAACAGGGCGAGGCGGGCGGGGGCGGCTCCCCGCG
CCGCCTCGGCCTCCTGGGCAGCCCCCTGCCGCCGGGCGCGCCCCTCCCTGGGCCGGGCTCCGGC
TCGGGCTCCGCCTGCGGCCAGCGCTCCTCGGCCGCGCACAAGCGCTACCGCCGCCTGCAGAACT
GGGTCTACAACGTGCTGGAGCGGCCCCGCGGCTGGGCCTTCGTCTACCACGTCTTCATATTTTT
GCTGGTCTTCAGCTGCCTGGTGCTGTCTGTGCTGTCCACTATCCAGGAGCACCAGGAACTTGCC
AACGAGTGTCTCCTCATCTTGGAATTCGTGATGATCGTGGTTTTCGGCTTGGAGTACATCGTCC
GGGTCTGGTCCGCCGGATGCTGCTGCCGCTACCGAGGATGGCAGGGTCGCTTCCGCTTTGCCAG
AAAGCCCTTCTGTGTCATCGACTTCATCGTGTTCGTGGCCTCGGTGGCCGTCATCGCCGCGGGT
ACCCAGGGCAACATCTTCGCCACGTCCGCGCTGCGCAGCATGCGCTTCCTGCAGATCCTGCGCA
TGGTGCGCATGGACCGCCGCGGCGGCACCTGGAAGCTGCTGGGCTCAGTGGTCTACGCGCATAG
CAAGGAGCTGATCACCGCCTGGTACATCGGGTTCCTGGTGCTCATCTTCGCCTCCTTCCTGGTC
TACCTGGCTGAGAAGGACGCCAACTCCGACTTCTCCTCCTACGCCGACTCGCTCTGGTGGGGGA
CGATTACATTGACAACCATCGGCTATGGTGACAAGACACCGCACACATGGCTGGGCAGGGTCCT
GGCTGCTGGCTTCGCCTTACTGGGCATCTCTTTCTTTGCCCTGCCTGCCGGCATCCTAGGCTCC
GGCTTTGCCCTGAAGGTCCAGGAGCAGCACCGGCAGAAGCACTTCGAGAAGCGGAGGATGCCGG
CAGCCAACCTCATCCAGGCTGCCTGGCGCCTGTACTCCACCGATATGAGCCGGGCCTACCTGAC
AGCCACCTGGTACTACTATGACAGTATCCTCCCATCCTTCAGAGAGCTGGCCCTCTTGTTTGAG
CACGTGCAACGGGCCCGCAATGGGGGCCTACGGCCCCTGGAGGTGCGGCGGGCGCCGGTACCCG
ACGGAGCACCCTCCCGTTACCCGCCCGTTGCCACCTGCCACCGGCCGGGCAGCACCTCCTTCTG
CCCTGGGGAAAGCAGCCGGATGGGCATCAAAGACCGCATCCGCATGGGCAGCTCCCAGCGGCGG
ACGGGTCCTTCCAAGCAGCATCTGGCACCTCCAACAATGCCCACCTCCCCAAGCAGCGAGCAGG
TGGGTGAGGCCACCAGCCCCACCAAGGTGCAAAAGAGCTGGAGCTTCAATGACCGCACCCGCTT
CCGGGCATCTCTGAGACTCAAACCCCGCACCTCTGCTGAGGATGCCCCCTCAGAGGAAGTAGCA
GAGGAGAAGAGCTACCAGTGTGAGCTCACGGTGGACGACATCATGCCTGCTGTGAAGACAGTCA
TCCGCTCCATCAGGATTCTCAAGTTCCTGGTGGCCAAAAGGAAATTCAAGGAGACACTGCGACC
GTACGACGTGAAGGACGTCATTGAGCAGTACTCAGCAGGCCACCTGGACATGCTGGGCCGGATC
AAGAGCCTGCAAACTCGGGTGGACCAAATTGTGGGTCGGGGGCCCGGGGACAGGAAGGCCCGGG
AGAAGGGCGACAAGGGGCCCTCCGACGCGGAGGTGGTGGATGAAATCAGCATGATGGGACGCGT
GGTCAAGGTGGAGAAGCAGGTGCAGTCCATCGAGCACAAGCTGGACCTGCTGTTGGGCTTCTAT
TCGCGCTGCCTGCGCTCTGGCACCTCGGCCAGCCTGGGCGCCGTGCAAGTGCCGCTGTTCGACC
CCGACATCACCTCCGACTACCACAGCCCTGTGGACCACGAGGACATCTCCGTCTCCGCACAGAC
GCTCAGCATCTCCCGCTCGGTCAGCACCAACATGGACTGA
EXEMPLARY HUMAN KCNQ4 ISOFORM A cDNA SEQUENCE
(WITHOUT STOP CODON)
(SEQ ID NO: 90)
ATGGCCGAGGCCCCCCCGCGCCGCCTCGGCCTGGGTCCCCCGCCCGGGGACGCCCCCCGCGCGG
AGCTAGTGGCGCTCACGGCCGTGCAGAGCGAACAGGGCGAGGCGGGCGGGGGCGGCTCCCCGCG
CCGCCTCGGCCTCCTGGGCAGCCCCCTGCCGCCGGGCGCGCCCCTCCCTGGGCCGGGCTCCGGC
TCGGGCTCCGCCTGCGGCCAGCGCTCCTCGGCCGCGCACAAGCGCTACCGCCGCCTGCAGAACT
GGGTCTACAACGTGCTGGAGCGGCCCCGCGGCTGGGCCTTCGTCTACCACGTCTTCATATTTTT
GCTGGTCTTCAGCTGCCTGGTGCTGTCTGTGCTGTCCACTATCCAGGAGCACCAGGAACTTGCC
AACGAGTGTCTCCTCATCTTGGAATTCGTGATGATCGTGGTTTTCGGCTTGGAGTACATCGTCC
GGGTCTGGTCCGCCGGATGCTGCTGCCGCTACCGAGGATGGCAGGGTCGCTTCCGCTTTGCCAG
AAAGCCCTTCTGTGTCATCGACTTCATCGTGTTCGTGGCCTCGGTGGCCGTCATCGCCGCGGGT
ACCCAGGGCAACATCTTCGCCACGTCCGCGCTGCGCAGCATGCGCTTCCTGCAGATCCTGCGCA
TGGTGCGCATGGACCGCCGCGGCGGCACCTGGAAGCTGCTGGGCTCAGTGGTCTACGCGCATAG
CAAGGAGCTGATCACCGCCTGGTACATCGGGTTCCTGGTGCTCATCTTCGCCTCCTTCCTGGTC
TACCTGGCTGAGAAGGACGCCAACTCCGACTTCTCCTCCTACGCCGACTCGCTCTGGTGGGGGA
CGATTACATTGACAACCATCGGCTATGGTGACAAGACACCGCACACATGGCTGGGCAGGGTCCT
GGCTGCTGGCTTCGCCTTACTGGGCATCTCTTTCTTTGCCCTGCCTGCCGGCATCCTAGGCTCC
GGCTTTGCCCTGAAGGTCCAGGAGCAGCACCGGCAGAAGCACTTCGAGAAGCGGAGGATGCCGG
CAGCCAACCTCATCCAGGCTGCCTGGCGCCTGTACTCCACCGATATGAGCCGGGCCTACCTGAC
AGCCACCTGGTACTACTATGACAGTATCCTCCCATCCTTCAGAGAGCTGGCCCTCTTGTTTGAG
CACGTGCAACGGGCCCGCAATGGGGGCCTACGGCCCCTGGAGGTGCGGCGGGCGCCGGTACCCG
ACGGAGCACCCTCCCGTTACCCGCCCGTTGCCACCTGCCACCGGCCGGGCAGCACCTCCTTCTG
CCCTGGGGAAAGCAGCCGGATGGGCATCAAAGACCGCATCCGCATGGGCAGCTCCCAGCGGCGG
ACGGGTCCTTCCAAGCAGCATCTGGCACCTCCAACAATGCCCACCTCCCCAAGCAGCGAGCAGG
TGGGTGAGGCCACCAGCCCCACCAAGGTGCAAAAGAGCTGGAGCTTCAATGACCGCACCCGCTT
CCGGGCATCTCTGAGACTCAAACCCCGCACCTCTGCTGAGGATGCCCCCTCAGAGGAAGTAGCA
GAGGAGAAGAGCTACCAGTGTGAGCTCACGGTGGACGACATCATGCCTGCTGTGAAGACAGTCA
TCCGCTCCATCAGGATTCTCAAGTTCCTGGTGGCCAAAAGGAAATTCAAGGAGACACTGCGACC
GTACGACGTGAAGGACGTCATTGAGCAGTACTCAGCAGGCCACCTGGACATGCTGGGCCGGATC
AAGAGCCTGCAAACTCGGGTGGACCAAATTGTGGGTCGGGGGCCCGGGGACAGGAAGGCCCGGG
AGAAGGGCGACAAGGGGCCCTCCGACGCGGAGGTGGTGGATGAAATCAGCATGATGGGACGCGT
GGTCAAGGTGGAGAAGCAGGTGCAGTCCATCGAGCACAAGCTGGACCTGCTGTTGGGCTTCTAT
TCGCGCTGCCTGCGCTCTGGCACCTCGGCCAGCCTGGGCGCCGTGCAAGTGCCGCTGTTCGACC
CCGACATCACCTCCGACTACCACAGCCCTGTGGACCACGAGGACATCTCCGTCTCCGCACAGAC
GCTCAGCATCTCCCGCTCGGTCAGCACCAACATGGAC
EXEMPLARY HUMAN KCNQ4 ISOFORM B cDNA CODING
SEQUENCE
(SEQ ID NO: 3)
ATGGCCGAGGCCCCCCCGCGCCGCCTCGGCCTGGGTCCCCCGCCCGGGGACGCCCCCCGCGCGG
AGCTAGTGGCGCTCACGGCCGTGCAGAGCGAACAGGGCGAGGCGGGCGGGGGCGGCTCCCCGCG
CCGCCTCGGCCTCCTGGGCAGCCCCCTGCCGCCGGGCGCGCCCCTCCCTGGGCCGGGCTCCGGC
TCGGGCTCCGCCTGCGGCCAGCGCTCCTCGGCCGCGCACAAGCGCTACCGCCGCCTGCAGAACT
GGGTCTACAACGTGCTGGAGCGGCCCCGCGGCTGGGCCTTCGTCTACCACGTCTTCATATTTTT
GCTGGTCTTCAGCTGCCTGGTGCTGTCTGTGCTGTCCACTATCCAGGAGCACCAGGAACTTGCC
AACGAGTGTCTCCTCATCTTGGAATTCGTGATGATCGTGGTTTTCGGCTTGGAGTACATCGTCC
GGGTCTGGTCCGCCGGATGCTGCTGCCGCTACCGAGGATGGCAGGGTCGCTTCCGCTTTGCCAG
AAAGCCCTTCTGTGTCATCGACTTCATCGTGTTCGTGGCCTCGGTGGCCGTCATCGCCGCGGGT
ACCCAGGGCAACATCTTCGCCACGTCCGCGCTGCGCAGCATGCGCTTCCTGCAGATCCTGCGCA
TGGTGCGCATGGACCGCCGCGGCGGCACCTGGAAGCTGCTGGGCTCAGTGGTCTACGCGCATAG
CAAGGAGCTGATCACCGCCTGGTACATCGGGTTCCTGGTGCTCATCTTCGCCTCCTTCCTGGTC
TACCTGGCTGAGAAGGACGCCAACTCCGACTTCTCCTCCTACGCCGACTCGCTCTGGTGGGGGA
CGATTACATTGACAACCATCGGCTATGGTGACAAGACACCGCACACATGGCTGGGCAGGGTCCT
GGCTGCTGGCTTCGCCTTACTGGGCATCTCTTTCTTTGCCCTGCCTGCCGGCATCCTAGGCTCC
GGCTTTGCCCTGAAGGTCCAGGAGCAGCACCGGCAGAAGCACTTCGAGAAGCGGAGGATGCCGG
CAGCCAACCTCATCCAGGCTGCCTGGCGCCTGTACTCCACCGATATGAGCCGGGCCTACCTGAC
AGCCACCTGGTACTACTATGACAGTATCCTCCCATCCTTCAGCAGCCGGATGGGCATCAAAGAC
CGCATCCGCATGGGCAGCTCCCAGCGGCGGACGGGTCCTTCCAAGCAGCATCTGGCACCTCCAA
CAATGCCCACCTCCCCAAGCAGCGAGCAGGTGGGTGAGGCCACCAGCCCCACCAAGGTGCAAAA
GAGCTGGAGCTTCAATGACCGCACCCGCTTCCGGGCATCTCTGAGACTCAAACCCCGCACCTCT
GCTGAGGATGCCCCCTCAGAGGAAGTAGCAGAGGAGAAGAGCTACCAGTGTGAGCTCACGGTGG
ACGACATCATGCCTGCTGTGAAGACAGTCATCCGCTCCATCAGGATTCTCAAGTTCCTGGTGGC
CAAAAGGAAATTCAAGGAGACACTGCGACCGTACGACGTGAAGGACGTCATTGAGCAGTACTCA
GCAGGCCACCTGGACATGCTGGGCCGGATCAAGAGCCTGCAAACTCGGGTGGACCAAATTGTGG
GTCGGGGGCCCGGGGACAGGAAGGCCCGGGAGAAGGGCGACAAGGGGCCCTCCGACGCGGAGGT
GGTGGATGAAATCAGCATGATGGGACGCGTGGTCAAGGTGGAGAAGCAGGTGCAGTCCATCGAG
CACAAGCTGGACCTGCTGTTGGGCTTCTATTCGCGCTGCCTGCGCTCTGGCACCTCGGCCAGCC
TGGGCGCCGTGCAAGTGCCGCTGTTCGACCCCGACATCACCTCCGACTACCACAGCCCTGTGGA
CCACGAGGACATCTCCGTCTCCGCACAGACGCTCAGCATCTCCCGCTCGGTCAGCACCAACATG
GAGTGA
EXEMPLARY HUMAN KCNQ4 ISOFORM XI cDNA CODING
SEQUENCE
(SEQ ID NO: 4)
ATGCCGGCAGCCAACCTCATCCAGGCTGCCTGGCGCCTGTACTCCACCGATATGAGCCGGGCCT
ACCTGACAGCCACCTGGTACTACTATGACAGTATCCTCCCATCCTTCAGAGAGCTGGCCCTCTT
GTTTGAGCACGTGCAACGGGCCCGCAATGGGGGCCTACGGCCCCTGGAGGTGCGGCGGGCGCCG
GTACCCGACGGAGCACCCTCCCGTTACCCGCCCGTTGCCACCTGCCACCGGCCGGGCAGCACCT
CCTTCTGCCCTGGGGAAAGCAGCCGGATGGGCATCAAAGACCGCATCCGCATGGGCAGCTCCCA
GCGGCGGACGGGTCCTTCCAAGCAGCATCTGGCACCTCCAACAATGCCCACCTCCCCAAGCAGC
GAGCAGGTGGGTGAGGCCACCAGCCCCACCAAGGTGCAAAAGAGCTGGAGCTTCAATGACCGCA
CCCGCTTCCGGGCATCTCTGAGACTCAAACCCCGCACCTCTGCTGAGGATGCCCCCTCAGAGGA
AGTAGCAGAGGAGAAGAGCTACCAGTGTGAGCTCACGGTGGACGACATCATGCCTGCTGTGAAG
ACAGTCATCCGCTCCATCAGGATTCTCAAGTTCCTGGTGGCCAAAAGGAAATTCAAGGAGACAC
TGCGACCGTACGACGTGAAGGACGTCATTGAGCAGTACTCAGCAGGCCACCTGGACATGCTGGG
CCGGATCAAGAGCCTGCAAACTCGGGTGGACCAAATTGTGGGTCGGGGGCCCGGGGACAGGAAG
GCCCGGGAGAAGGGCGACAAGGGGCCCTCCGACGCGGAGGTGGTGGATGAAATCAGCATGATGG
GACGCGTGGTCAAGGTGGAGAAGCAGGTGCAGTCCATCGAGCACAAGCTGGACCTGCTGTTGGG
CTTCTATTCGCGCTGCCTGCGCTCTGGCACCTCGGCCAGCCTGGGCGCCGTGCAAGTGCCGCTG
TTCGACCCCGACATCACCTCCGACTACCACAGCCCTGTGGACCACGAGGACATCTCCGTCTCCG
CACAGACGCTCAGCATCTCCCGCTCGGTCAGCACCAACATGGACTGA
EXEMPLARY HUMAN KCNQ4 GENOMIC DNA SEQUENCE
(SEQ ID NO: 5)
GCAGGACCACAGCACTGTTGCCTTCTGCATGGTCTTCCAGAGGTAGAGGTTAGAGGAAGGATCT
TACAGGAAGAATCCTGAAGGCAGAGGAGGAGCCCTGCAAGAAGGGAGGGCTTTGACCTTCATTT
CCACGCCCCTTTCTCCCTCTGTGTCTCCTCTGTGTGGGCCCCCTCCAGCCCTGAGGATCAATGG
GGGTTCACTTTCACTGAGCTCAGCAACACTGCTCCCTCCCCACTTCCTGTGGCTCTGAGCTGAC
TGGCCTGCTTGGTCTAGGCTGGGGGCTGGTCAGTGCCACACCCAGATCTGGGGAAAAGAAATTC
CTTGGCTGGCGAGGTGGCTCACGCCTGTAATCCCAGCACTTTGGGAGGCCAAGACGGGCGGATC
AGGAGGTCAGGAGATCGAGACCATCCTGGCTAACACGGTGAAACCCCGTCTCTACTAAAAATAC
AAAAAAATTAGCCAGGCATGGTGGCGGGCGCCTGTAGTGCCAGCTACTTGGGAGGCTGAGGCAG
GAGAATGGTGTGAACCCGGGAGGCAGAGCTTGCAGTGAGCCGAGATTGCGCCACTGCACTCCAG
ACTGGGCAACAGAGCGAGACTCCATCTCAAAAAAAAAAAAAAAAGGAAAAGAAAAAGAAATTCC
TTGGGCCCCTCCAGCACCTCGTGGCCTAAGTCTCTTGTCCTTTCTGGGCTGCACAGGGAAAAGG
AAGAAGGCCTTATCTTTCTCTCATGCTTCCTCCTGCTCTCAGTAGAGGTGGGTTCCCAAAGGTC
AATAATGTGATGCCATCACCCCCTGAGCACTCTCCACTCCAATCCCCTCCTAGGAAGGCTTCCT
AGATTGAGAAGAACTGCTAGGACCAGGGAATTCCCAGAGCATCTCCCTGCTCCACCTGAAACAC
GTACCTGAAGAGCAGTCAGACGAATGGGTCACGTGCGTACAGCCCCCTGCATGGTCTCTAACTT
TTTCATGCATTATTGCATTCAGTGCTGATAGCCAGGGAAAGTGGGGAAGAGGTGGAGTGACTTG
CCCAAGGTCCCCAGGTATTCCACAGGTGGAGCTAGGACTGGAAGCCCAGCCATGACTTGTAGTT
TAGGGCTCTTTCTGCCCCTGGCACTGCCAGCCTCTCTGGATGCTGGTGCCTCCCACTTCACCCA
TCCTGGGTTCTCTCCTAGGGAGCCACAGCCTCTGCAGCATGCCCAGCCTCAGGGCCTGGCCAGG
CTCCCTGTTTCTTATCAGGCCAAATTGGACCCTCCATCCTGGCATTCAAGTCCTCCTGCGTGCC
CCTGGCTAAGCTTGCTCTTCCGAATGCTCTGTAACATTCTTCCCACACCAGTACACACACACAC
ACACACACACACACACACACACACACACACATTAAAGCAGCACCTGAGTGAATTAACAGCCATA
CAAGGCAAGTCTCTTCACTGAGCCTCAGTCTTCTTGAGTGTAACATGGGACCGTGAACAGTATT
ACCGCACAAGGTTGTGGGGATGCTGAAATAGACAATGCAGGTAAAACATCCTGCATAAGGCTTA
GGATTGACTGCAGGCCTCATTAATGGTAGCTGCAGGCATTATTGGTTGTACTTCTCTGCGTCTC
TCCCAGTCCTCCCCACTCCTGTCCCCAAGAAAGCTCCCTCTGCCATTGCCTGTGGAGACTTCTC
CCTCCTCTGAGCTCTAGCAACCCTTGGAGCTGTAAGAAAGCCCAGGGTCAGGTGGCTAGCAGGC
AGAGAGATTTTAGGAGGCAAGTGAGTGGAACCCAGACCTCCTCGCCTCGATTCAACTAGCGCAC
TTCTGCTCCAAAGCTTTGTACACATTGGGGTGCTACCTTACAATTCTGTCGGTAAAAAGGATTC
CATTGCTGATAAAGTCTAAAAATCACTGAGTTAAACCACCCCACCTCTCACTCGAGGAAAGGAA
CCTGAGCCCTTGATTGACCTGGGGTCACAGTGGGAATCACTGACAGGCTTGAGCCTAGCCTGAG
AGACCATAGTCCTAGCCACTTCCTGCCCTCCATCCTTTCAGACCTGGAAGTTTGATCCCTTGGC
ACGAGTAAGTTCTAGAGAACAAGGATTGGCCGTGTATGGTTATCCTCAGCTCCCAGCACCCAGT
GCCCAGCCGGCAGGTGCCAGTCAGTGGAAAGCACAGGTCACAGTTCTGGGGGTCACCAATAGGC
TGTGTAGACTTAGGTGAGTCCCTGTGCCTCTCAGAGCCTCAGCTGTCTCAAGCAAGCTGCTGCT
CTCTGGCCCAAAAGGGACTGGGCCTGGCACACAGATGGCACTCAGCAAATGGAAGCACTCGCCC
TCCCACCCTTCCTCTCAGTCAATACTTGCTCCATTGATGGCTGATGGGGACAGTGATGAATTAC
AGAAGATCTTGGAGCCCTGCCCACTTCTTGCACAGATGGGGAAACAAGTCCAGGGTGGGGAAGA
GACTTGCCCAGGTGGGAGTGAGTCTTAGTGAGTGCGTCTGTCTCCTCAGTGGGGCAGGGAAGCT
TTCGCAGTGTTACCAGGGTGGAGACAGGGCCTGGGGGTGGAAAGGAAACTGCTGAGGGTGGGGT
CAGGGGAACAGGTGGGAGAGGGAAGGAGGAGGGTAGGGAACCACGGTGTCCAGTCCCCTCCAGC
AGAGGGGCCAATTGAGGGAGCCTGAGACAGCTGTTTGCTCAGAAAAGTGTCTTAGTCACTAAAG
GTTGTGGTGGGGAAAGTCCGTCCTCCCAGTCATGTCCTGGGAATCCGGATGGCGCAGGAAGGCC
ACCCGGTGACCCTAAGAGTGGCCACCTGTCCTCTCTGAACTGGACTTTCTCTTCTGGCCCTTCC
ACTCTTCCATGTGTACACATATGCCAAGTAAATTTGCTGTGGCCTCCTGGAACCATCTCCCCCA
TCCCTCTCACCCCTTAGGAAGAGTTCTGTGTCCCTCCATTACCCATACGCCCGCAGGAGAAACA
GGCTTCTATGCCCTATGAAAGTCCTTCCTGAGATCTAACTTCCATCCCTCTAGCTGCAACCACA
GTCTCTTTCCTCTTTCTGCTGCCTTTGAGCAGGTAATCTGGAGCTGGAGTTGGGAATGATGAGA
CACTCCCTGCAGTGAGGGCTCCTCACAAGGTGGGGAAGGGACCTTTGTAGTCCCCCAGCTGGGA
AGAGAGCCATGGGATCCAGAGGTGATGCTGAGCTTGAAGATTAGATTTCCCAGAACAGACCCAG
TTTCAGATATTCTGGGTGTTAAGGGCAACCTGAATTCAAATTCCATTTCTGCTACAGAACAAGA
GCCTCAAAAAGACCCCCTAGAAACACCCCCAGGGCGCTTGCAACTGCTTTCCTGTGCACCTTTC
ATGTCCTAGACCTTAGTCCCATTTCCCAGGATCTATGATTGAGAAGAACCAGCCAGGCAGTTCC
ACAGAGGGAGGACAGTGCTGCATATTTTGGGGGTGCCATCTTCAGCTCATAGAGAGAAGATGGG
TTAACTCAGCCCAGGCACCACATCCCTAGTGGGGCCTTGGGGTCCAGAAACTGTCCCCAGCTTC
CAGTAGAGGATCCGGGCATAACCTGCCCTGGGGCACCCGGCTCTGGCCCTGGGCACACCTGGAC
CAGGAGCAGCAGAAGAGATTCAGGTCAGACCCCAGAGGGCACCCCCAGCTAGGCAGAGGGGAAA
GGATCACTATGGGGTAGGGGTGTGGGCCGGTTGCCAAGCCCAGTTTAGGGACCCCACAGATCCC
TGCAGCCCACGGCCCTGCCTCCCTCCCTCCCTCACTGGCGCTCAGCAGATCAATGCTGCCTTTG
CTGACAGCTGAGAATCGAGCTCGCCTTCCCGCCCCTTCCCCCGCCCCTCCCGCTCGGCTTCGTC
CCTCGAGATCCTCCCGGAGGAACCGGGAAGAGTTTGCTGCGGAAGGCTCACCCTGGGGCAGGGC
CTGCGGAGGGAGCGGCTGGTGTGGCCGCAGCTTTCCGTGGAGGAAGAGGGAAAGAGGATCGGGA
AACCCAAGTTACCAACCCTGTGCAGGGCCCAGGAGGGCTAAAGGAGAGGGACTGCCCCCTACAG
CCAGAGAGATGGAGGTCGGGGACTAAGAAAAACTTTCCAGCTGGAGAGGGAATGCCCTGGGACA
AAGTATCCCCTTTTCTGGCTGGGATTCTGTGGGCCTTGGGTAGGGGGTGTGTGTCCCCTCTAAG
GCTGGCTTAGAGCTCATCTCCCAACACTCCATCTGCCCAAACCCAGTTCCCTGACCTGGGAGTG
GGGCAGGGGAGCAGGCCCCTCCATCGGGGAGGATCCTCTTCTGGGCAGGGTGGCTCTTCCCCCA
TCACGATGAAGGGAAGCTCAGAGGTGGCAGTTGGTGGGAGAAAGACACACCCAGGCCCACTGTG
TTCCACACACCCTGATGCTGCCTAGCAGCCCGTGGGCTCCAGGCCTTTCAGGTAAGAGCAGGAC
AACCGCCGCTGCCCACCCAGCCACACACAGCACTGGGCACACTTTAAGCACCCGCACCAGGCAC
ACAGTGCTCGACCCCAACGGACACACCTCATCCCCTTCGGACTCCACCTCCACTTTCGCAGCCA
CCCACACCCGACCCATAACTCACACCTCACTGACTCCATCCCCGCACCTGGTAGACCCACAGCC
ACCCACCCTGCACACACAAGTAGACGCCGTTCCACCGGTCTACACCTAAACTCCACGGGACACT
GCGTCCCTTCCCCTGCCGCCCGCGGCCACAACTCCACATTCCACTTTCCACAAGCACACACGAG
GCGTCCCACCCCGGGCTGGCCTTCGGGGCCACACTGCCCCACGCAGCGGTGGACACCACTTGCA
CGCGTCCGGCTTCCCGGCCCCGCGCGCTGCCCCCGCCACGCGGTTCGGCCCAGGCACCAACTCG
GCCGCCCGTGCGCCCTGCCCCGCCGCCTGCTCCGCGCGTTCCCTCCCTCCGCCTCGCCTCGCTT
GCTCGCTCGCTCCCTCCCGATTTGGGAAGGCGGCCGCGGGGCGGGCGGGGGAGGGGCGGGGCGG
GGGAGGGTGACATGTGAGCGGCGCGCGCCGGTGGCAGGTGGAAAGGCGAGCGGCATGGAGCGCG
TAATAAGAGAGTTGGAGTCGGAAAGAGCAGCCCCAGTCGCCGGGGAAGCGGGAGGTCAGTGCGG
GCTCCGGCGGCCCCCAGGCTCCGAGCGCCCGCCCGCGGCCCCGGCCCGGCCCCTAGCCCCCGCC
GCCCGCGCCCGCCCCGGGTCGCCCCTCTGGCCCCGGGTCCGAGCCATGCGTCTCTGAGCGCCCC
GAGCGCGCCCCCGCCCCGGACCGTGCCCGGGCCCCGGCGCCCCCAGCCCGGCGCCGCCCATGGC
CGAGGCCCCCCCGCGCCGCCTCGGCCTGGGTCCCCCGCCCGGGGACGCCCCCCGCGCGGAGCTA
GTGGCGCTCACGGCCGTGCAGAGCGAACAGGGCGAGGCGGGCGGGGGCGGCTCCCCGCGCCGCC
TCGGCCTCCTGGGCAGCCCCCTGCCGCCGGGCGCGCCCCTCCCTGGGCCGGGCTCCGGCTCGGG
CTCCGCCTGCGGCCAGCGCTCCTCGGCCGCGCACAAGCGCTACCGCCGCCTGCAGAACTGGGTC
TACAACGTGCTGGAGCGGCCCCGCGGCTGGGCCTTCGTCTACCACGTCTTCATGTGAGTTTGCG
ACCCCGCGCCCTTCCGCGTTTCCCCGCGCAAGCCTGGCCTCCCGGGGCACGGCCGCCCCGCCCT
GGCTCCGCCTTCTACCCCCCTGCCTCAGGGCCGACCCTCATCTCTCTCCCCCCAGGCCTAAGCC
CGGTTTCTGATCCCCTCGCTGAGCCCGACCCTAAGCCCTGATCTCCCAGGCCTGACCCCTGCTT
GACCTCGCTTCTGACCTCCCCGCACCGCAACTGCTTATTTCCATCCTGACCGCCAATCTCTGCT
CCTCTGTCCCAGGTACTCCCGACCGCCAGCTGCCTCCCCCAAGTCCGCTTGGCGAAGTCTGGGG
CCCCTGGAGTGGGCGCCCTTTTCCTCTTCCCCACCCCTGCCTCCCTGTGTCCCAGACACAAAGA
GGAACCCTTGGCTCCCACACGCTTCTGTCACCTGCTCCCCAGCTCTGAGCTATGAAGGGCTCCC
CTCAGGGGTGCTCCTCTCCAGGGCGGCCCTCATTCCTACCCCTGCCCAGCTGACTGTGGGTGTG
TGGGGGTCCCTGTGGGCCCCCTGGGCCCTGACACTCGCATATGCTGTGTCCGACTTGATCTGTG
TCTTCGCTCAGTCTTCCCCTCCACCCTCAACTTCCCAGTGGCAACGGCGGCCGCCCTGCCTGCC
CTGCCCTACCCTGCCGGCTTGGCCCAGCTCGCCTGCCTTGTTGGCTGGCAGCACCAGGCCTGGC
TCTAGGCACCAGGGGAAAAAGAAAGAGGAGAGGGGTAGGTTTGGAAACACTGACGCTGCTGGGG
CCCCCTCCTCCCTCTCACCAGGCTCCTACAACTTTCAAAAACACTGACCCCTCTTGGGTGTGAA
GAGTGAATTTTGGCAGCTCAGAGCTCAGTCTTGGGGTACGGCCTGGCCCAGCCAGGAACATGAG
AGGCCCCGCAGATATCAGTTGAAGGGCTGGGCCTGACGCTGGGACGCTCAGAGGTGCCCAGAGA
GAGACCTCTGGTCCCTGGAAAATGTGTGGCTATTAATCCCTTTTTGCTGCCACCAGAGAGGGCC
CTTTGGCGCTGGGGTCTCCTGGGTGGCCCACCTGCCTGCCCACCTGACTGTCTAGCAGCCCTCC
TGCTGGCCTGGCTGTCCCTCTGCCAGCCTGAGTTTCCACCTGTGTGCGAGTTGGCCAGTCTGCT
CACCCTTCCACCTAACTGTCCGCAGTCCTTCTGCTTGCCTGTTTCCATCCATCTGTGGCCTGTG
CAGTTATCCGGGTGCCTGTCTGTGCATCTGCCTATCCACCTGTCCACCTGACTGTCCACTCTTC
CTCTACCTGTCTTTTCCACCTGTCTCTGGGTCTGTCCTTCGGCCTGTTTCTCGGGTGCTTGCTG
GCCTGCCTCTGCCCCATCTGAACTGGGCCCACATAATTTCAGAGGTGAAGGGGAGAAAGATGCG
GCACAGGGAGGGGCAGGGGGGGAAGACAGCAGGGCTCTCTCGGCCATTAAGTGCGTTGGGGGTG
GCAACCTTGACTCTGTGCAGAGACCTCAGCTGGGCTAGGCTGGGGCAGAGAGTTGAAATTGCTC
AGGACATGAGGTCAAGCTTCGGTTCAGGGTCAACGGTTTGTAGTAGCCCAGTCCTTAGTGTCCA
TTGGGAGGCCTAGGATGAATATTTGTGGCTATAAAGAGGCCCGTTGGAGTGAGGAAGGCCCAGG
GCTGGTGGAGTTGGGGGTGAGGGGATGGGGACTACTTCTTAGGAGTGGCTACAGAGCAGCCTCC
TTGAGTGCCTAAGGAGGGTGAGACACCCCCCAGAGAACAACTGTGCCCTCCGTGGGCTCCTCTT
GGGAAGGGACTTTTCCAAGGCATGGCAGGGCCTGGGGAAGCCAATAGGGCTCTGAGGTGGGGTC
CTTACCCCCCCAGGCTTGGCCTTACAGCCCCCACAAGGGTCAGTGGAGGTGAAGGACCTGCCTC
CTGTCCCCTGGCCAGCTGCTGTTGGTGCCTCCTTTGAAAGGACCGCCCCTTCCTCTCCAGGAGC
TAATCCACTGGCACCTTTGCCCCACATCCTGCCCTTTCCTGATGCCAGGAAATGCCCTCGCCAG
GGCCGTGCTTGTCCTGACCACTTGTCCCCGGGAGCCCAGGGCCAGGGCATGTCTTGGGAACTCT
GGGGGAAGAAGTATGGTGTGAAAGTGTCCTGGTGTTTAAGGATCTCAGGGAATTGGTCACCCAT
CTTAGAGGTCAAAGGGGACAGAGGTGTCAGGAACCCCACCTCTTATGTGCTCTGTGACGTTGGG
TTGATTGCTGGGCCTCTCTGAGCTTTAGGTCTGTAGGTTGTCAAAGCGGGTAGAAACCCCAACC
TTGCGGCCTCTCTCTTGGATGAGGTAATAGAGTGGGAAAGAGCTTGGTAAGCTCCAGGGGCCTG
TCTGAGCTTGAAAGACGGTTGTTATTATTGGGGCTGGAGGAGGGGGCCCACACAGAAGGTCTCT
CCTGTTTGTGAGGAGCTCCAGATGTGGGGTTTCTCAGTCACACTGCAGGGTCTTGCCAGGAAGT
ACTTCCTGCTGTTGGACTCCTAGCCAGACCATTACACTTTGGGTCAAATCCTTTCTCCTCTTCC
CAATCTGCCTCAGTGGCAGCCCAAGTGAGGGGAGTCCAGGGAGTACTGTTAGCTAGGACCCCCT
TCCCAGAGACGCAGGCTGTGGCAGGAGGGGCCCCAGCAGAAGAGCCTGCAAGAGAAGGGCTCTG
GGCGGGGCGCAGTGGCTCTCACCTGTAATCCCAGCACTGTGAGAGGCTGAGGTGGGAGGATCGC
TTGAGCCTAGGCAACATGGTGAGACCCCTGACTCTACAAAATTTAAAAATTAGCCGGGCATGGT
GGTGCACACCTGTAGACACAATTACTTGGGAGGCTGAGGTGGGAGGATCACTTGAACCCAGGAA
TTCTAGGCTATTGTGAACTGTGATTGCACCACTGCATTCCAGCCTGAGTGACAGGATGAGACCC
TATCAATTAAAAAAGAGAGAGAAAGAGAAGGGCTCAGGAAGGAGGGAGCTGGGGTGAGAGGAGC
GCATTCGCCCTAGGGTATGCCTCCCTCCCATCTGCCAGCCCGTCCCCTCTGAGCCCCACACAGA
ACCTGCATCTTTCTGGCTCCAAAAGGTGGGAGATTGCCTAGAGTACAAGCCCAGATTTGGCCTG
TCCTGCGGCAGCTGCAAGCTCTGGGAAGAGAGGACTGCTGACTCCTGTCTTCTCCTGTTGGGAT
CCAGAATGAGGGGCCACTGAGCCCCTCTCCAAGCTTTCCCTCTCTCTGTGGACCCTCAGTCTGA
TCACCAAGCTGCATCCCCCTCCTATGCCCCAGTCCCTGATTTAAGCCCTGAAGCCTTCACAGGC
CCTGCTCTCTGAATTTTAAGAAATACCACCTCGTGGCCCATCCTGCTCGCTGAGCCCTTATGGG
GAATGCTCGCTGTCCCCTTGCTGCCCCCTCTCAAAGGCCACTCCAGCTTCCCTTGAAGTCAGAG
TGCTGGAGGGGGCTGAGCAAGACCCTATCTCCCTTCATCCTCCCCAGCCACTATGAGCCGCAGG
CCTGCCGCTCCCAGCCCCCAGGGCCCAGCAGACTTTCTCTATGTGAAGGTTTGCGGCTCTGTAA
ACATCCTCCCTCAGCCTTGTCCGTGGGGTCAGAAGCTGTGGGAATGGGGAGGGGAGGAGGTGGC
AGCAGCTTCTGGGGCCCGCAGACTCCTAACCCCCGAGCCCCCGACCTCCCACCCCGGCCATCTG
AGAAAGAACATCGCCTGGGTTGGAAATCATCCCAGCAAAGTGTCCTCAGAACCCTCAGGCATCC
CCGCTAGGTCAGTCGGTCCTCACATTTTGACAGCCCCATTTTACTTGTAAGAAAACTGAGGCTC
TGGAGACCAAAGTCACCTGGGAAATGAGTAGCAGATACAGCGGACTCTGTGCAGAGACCTCAGC
TGGGCTAGGCCAGGGCAGAGAGTTGAAATTGCTCCTGCTTCCTTGTCCAGGACTGTTCGCTCCG
TTCCTAGTGGCCACCGGGCTTGGCTCTTGGAGTCTGAAATGAAATCTCTGGTTGTGTGTGCAGC
ACCAACTATGCACAGGGCACCCTGCTGAGCTCTCTCTAAACCGGTCTCATTGTGTCCTTCTGCA
GCCTTGTGAGGTGGGCACTGTTATCCTCCTCATTTGACAGATGGGGAAACTGGGACTCAGCAGA
GGCCACCCAGTTAATGTGGAAGAGCCTCAGCCCGGAGCGGGGGCTGACTGGGAGCTGTTGCGCT
CAGCACCACAGCCTCAGCTAGCAGCACCCCAAGTCCAGGCTCCTCAAGAACAGCACTCCTGTGT
CATCTGTCCGTCCATCCCAGCACCCCGCACACAGTCAGCAGGGGGTGGGCAGTGATGGCATTTT
GTGGAATTGAGTGGAAACTGCCAGCCTCACCCAGTGCAGCCACGTTTTAGTTGTAGACCACGTC
ACTGCTGCGTACCTGTCTGAATGTCTCACACATGCACTCACTGCAACATCGACACACATGCACA
AACACACACACAGCATCCATCCAACAAAAAGAGTTCTAGTTTAGAGCTGCCCCTACCTTTTCCC
TGACTTCACAGACACTCACCATGGACACACACCCGGGCCCGTGGCTCCAGCCCAGACCCCTAGG
ACAGAACAAAGCTTGCTCCTGTCCTTGAGTTTTTGGCCTTCACCTATACACAACTGCAGGGCTG
CCCTTGCGGGTCTGTGTGTATTTGTGTAGCCACCTTGGAGGGCAGCCTCAGAACTGTGGAGGAG
CGGGGTGGCTGGCCTCGTAGAACTTCGCTGGCGTTTGCTCCTCCTGGGGGGTCCTCGGGGGTCC
TTTGATCTGACCTTGGGGTATACTGCTGGGTACACCAAGACACAGAAAGGGGCAGCTACTCATC
CAAGGTCACACCTAGTGAGTTAGTGGAGAACTGTGCCAGGAACCCAAGCCTTTTGTCTCCCAGA
TCAGGACTTTAGATAACAATTTTCCTAACCTAGCCAACAGGGGAGATGGATGCTCTAAGAACTT
TGCCCTTGTGATTGGGAAAGTCTTTGAGTGTGACCCAAATTTTTCGTGCTGCTGTTTTTCAGCC
CTAGTCCTTTTGCTTGGTCTTTGTAAGGAGCTGGGGAGACCATCCTGTTCTTATACTGCCTTCC
TCCTCCTCCTTGCACTCCCCTTCACGCTCCTTTGCCTCCTTCTTCTGAAGTTCTTTGGTGTCAC
TGGAGCCGGTTCGATCTCTCACTGACGGCCCACCCCACCCCAACTCCATAGTACGTTCATTCTT
CCATTGAAGACGCGCTGCTAGATGTCTAACTTCAATCCCTATTGCTGCCGAAGCCCTTATCCTT
GGGATATTTAAGCCAGGAGTGTTTCAAGAGGTGGTTATTATTCTTGGGCTGATGGCAGCACTTT
GAGTGGATGATTAATTTCAGGCTCTCTGAGCCAGTCAAAGGAGCTGGAACTGCCTGGGAGCAGG
GAAAAAGGAAGGCCGAGGCCCTGCCCACAAAGGAGGCTGCCCAGGGAACTTCCCCAGACTCCCT
CCTACCTCCCTAGCACCAATGTGGGTCTTTGGGGAAGGTCCGATCTTCTTGTGGGCTATGGAGC
CCCCCAGCAGGGAATTCTGCATGTTTGCCCTGGGGGAAGCTCCTAGTGTGGGAGTTGAGAGGTT
GAGGATGGGGGAATAGCAGGGACTTTTCGTTTCTCCAGAAGCTGTCCTGCGGGGAGTGAAGAGC
ACGGGCTCTGAGGCTGACAGATCTGCGTTCCTTCCTGGTTCTAGCCTTAGCAGTCGTGTGTCCT
TGGGCAAGTTACTTAACCTCTCTGGGCTTCAGTTTTCTCATCTGAAAAATGGGAGTAATCATAA
TACCTGCCTCTAAGATTGTCAGGACTGGATGAGATAAGGCACCGAACACTGCTGGGCACTGTTA
GCCATGATCATCGTTACTGTTATTATTATGCTGTTATCACTGTCCTCATTCTTCTCCTCCGGGG
AGAGGTGTGGTAGGTGTGCTGGGCTCAGCAAGGTGAGTGTGTCTGAGTCTGCTCTGGAGAGAAT
AGGAGCTATGTCTGCCTGTGTCCTGGGGCGAGTGGTTCTCCATGGGAGCTGAGGCTGTGTCTTT
TCAGCCTCCTGTGGGCCCTGCCCATGAGAGTGTCTGACTTACATTCTCTCGTGTAACGCTCACA
GCCATCTTATCAGGTGGGTGCTGTCTTCTTCATGTCACAGTTGAGCAAAGTGAGGCTGGGTGAG
GGGATGTCACCCAGCTCATGAATGGAGCCCAGCCTGGCTACCTAGCATGCTCACTGATTTGTTT
CTTCCTTCATCTGGCAAACAGTTACAGAGCATCAGCTCCATACCAGGACCAGGGAGCCAGAGAG
AGCTAGACCATGCCTGTACCCTGCCTGGCAGAGTGGGGGATTGGGGGTCACTGTGAGGAGGTCG
CAGAGTCTGCCGGGACCACTGGCCTCAGCTGCTTCTTCTCCCAGGGAGAGGTTTGGAGAGAGGT
CTGGTCTGTGTGTAGCTGTGAGTGTGTCTGTGAGTTATGGGGAGGACGGTGTATGCCAGAGGGC
CACTGTGCGGCCGAGTTTGGGCCTCATGGTCCAACCCTGGCAGCTGGCCTGGCCCCCGAGAGAT
AAAAGTAGAGCCATGGTGCCCAGAGAGACAGCCAGGGACTCCTGGGAGAGGAGGGCACTCCCCC
ACTCTCTCCTGAGCAAATGATCATGGAGAATGAGGGCAGGGAATCCCAAGGGAGATTTAGAGAT
CTCAGTCCCCATGCTTACTGGGAAGGTGTCCACCCCCACTCCACCCCAGTTCCTGAGCCTCGGT
ATCTTCTTCTGTAAAATGGGCCTAACAGGAAAGTGGACCTCTCAGGGCTGCCTGGCACACAGTC
GTGGTGGGCAAATGAGAGAGACTGCTGTCTCAGGACCTTGAAATGCATAGGGTACCAGGTAGGG
AGCTCTCTTCCAGCATCTTCTCTGGGGGCCCCTCAGGTGACAGGGTGCCACTTCACAGGGCACT
CAGGGGGCTGCCTGCCCCCAGTGGAGGCTCTGAGGGGCTGGAGAAAGGGTGCTGAGCCGACCTC
AGTGGCTACACAGAGCCCCCAGGACTTCAGGTTCCCTGGGCAACCCTGTCCCTGGGGCAGGAGC
TGCGTGCCCTGGGGATGAGGCACAGGAGGGGCAGCCCCAGGCAGCAGACTAATTGGGCTGGGAA
TCCGTGGGCAGCGCCTGAGGAAGGGAGGCAACAGGAGCTCAGCGATTCCCTCCCTCCTCACCCC
ACGGAGATTTGGGGGACAGTGGATAGAGCAGGGTCTCTAGCCTAAAGGATGGAGGGAAAATCTT
GAGGCACATTCTCCTGAGACTCCTTGGACACCCAGGCCGGGAAGGTGGGACTGGAGGAAAGCTC
ACTGGAAATAAATGGAGCATGGTTCCCATCAAAGCAGAGGAGATGGAGGTCAGACAGCAGGAAG
GACTGCCCAAGAACCAGGAGGTGAATGAGATTTTCTGGGATATTGTTTCAAAAACTTGGAGCCC
CCTGTCTAGCCGGGCTGGACAACATTTACTCGAATTGTCCCTATCAAGCAAGGAATGGCTCCCT
ACTCCCACAGAGCGAAAGATGGAGGGGAGATGCCTGTTCCCTTTGAGCCCAACTTTGGCGGGGT
GTGGGTCGGGGGTGGGGTAGGGATCAGATCGGGTACCCACTGGCTTTTGGGCAGTGGTGGGAGG
GTCACAGGCTGCTCCTCCTTCTCTGCCACTTCTCTCTGGGAGCCTCGGGCCTGTCCAGGTTGCT
GTCTTTGGGTTTATTTTTTGCTGGGCTTGGCTGTCCCCTGGGAGGGGAGGGGGCAGGGAGAGAA
AGGAAAAGCAGGAACAGCTTTGTCAGCACAATCTCAGGCGCCTTCGCTACTGCCCGTCTTGGCC
CAGCTGCCACCCTCTACTGGAGGGACCAGCCCCTGGGCACAGTGGGGATTCAGACCCTATGGAA
CTTCCTCGCTTAGAGGCAGGACCAGCAGGAAGGGACTAGGCCTTTCTACCCCCATCCTCTCTCC
ATATATCCCTGCTATACAGAACCTCAGGTCTCAGCGCTGGAGCAGATCCACGGGCTTCATTTCA
CAGGTGGGGAAACTGAGGCTAGAAGGGACCTGATGGCTCAAGGGCACAGCCCACACGGGGGCCT
TTTCCACCTTCCCTCCTTATGGAGTAACATGGGAGAAATGCTGCCTCCCTGCTGCCCGTTCCCT
GGGTGAACTCATTCACTCATCTCATTTTCCAGCCTCTGGGGATTGGTGGCAGTCTGGTGGGAGA
GACGGGGGACGGGGAAAGGACAGTGTGACCCATGGCCCAGCAGAGGAAGGGAGGTACAACCTGG
GGATGCACAGAGGCTAGGTGGGGGTATCAATCCCTCCCCTATATGCTTTTTATCCTAAAAAAAT
ACATTCGCCCTATTTTACAGACAGGGGGATTAAGGTGTAAAGAAGTTAATGACTTGCCCAAAGT
CAACATAGCTGACAGGTGGTAGAGGCAGGATTTGAACCTAGCATGTGCATCTCTGGGAGCCATG
CATTTGGCCACTCCACCATGCTGCCTCCCCAAGGGGCCCCTGTGGGCCCTTCTTTCTTTTCTTT
TCTTTCTTTCTTTTTTTTTTTTTTTTTTTTGAAGCAGGGCCTCACTCTGTCACCCACGCTGGAG
TGCATGATCACTATGATCACTGCAGACTCAACCTCCTGGGCTCAAGTGATCCTTCCACCTCACC
CTCCTAAGGAGCTGGGACTACAGGCGCACACCAAGACACCCAGCTAGTTTTTAAATTTTTCTGT
GGAGACAGGGTCTTGCTATGTTGTCCAGGCTGATCTCCAACTCCTGGCTCCAGCGATCCTCCCA
CGGTGGCCTCGGGAGCCACCATGGCTGCCTGCTGGGCCCTTCCTTCTTGAATGCAACCCCCCTG
GCTTTCCTCAAACCTGCCTCTGTGGCTTTTCTCTCTTGGTTCTATCAGCAAGCAGCTCTGCCTC
CTCTTTCTGCCCCTTGAATGTTGGGGCTTTGTCTCAAGTCCTCTTCACAGTACATAGTTTCAGG
GGGTGGTCTCATGCACTCCTACGACATCAGGGGTCACCCCTATGATGACAGCCTCCGCTCTGCA
GTTTCATCCCTGCGCTTCCTGCCTGCTGACACCTCTCCTCCTCATTCCCTGGGCATCTCAAACT
CAGCATCTCCCTCCAAACCTGCCCTCCTTCCTCAATCCATGTGACAGTGACTCCCCGATCCCCA
GTGCCCAAGTCAGAAATGCCCTCCACACAAGCATCCTTTGCTCTCACCTAAGAAATGACTTACA
GTGTCCCAAAGCATTGCGCTGTTTAGTGCTTCTGTGCTTTTCCTTCTGCCTGCAACTCCCTCCC
CTCTTCTGCCCACTGCATCATCTAGCTAACTCCTATTCATCCTTCAACTCCCAGTTTGAGCACC
ACCTCTTCTTGGAAGCACTCCCTATCCCCATTACCCCCAGGCTCAACTGAGGGCCCTTCAGTGC
CCCCAGAGTTCTGTTCACCCAGCCCTGTGCGTTTTTTGCTTTTCTGCATGGGACTATGAGAACT
GGGTGTGTGGGGGTTGAACACAATTCTTGGCACATGGTCAGCAATGAGTGAATGTTTGATGAAG
GTGGCATTGGGGCCAAGTAGTAAGGATGAGAAGGGTTTTGCCAACTAGAGAGAAGTGGAGAAAA
GACCCCTCAGCCCTCTGTGTGGAGCACTGAGCTGTGGGTGTTCATGATTTTTATCCCGAGTGAG
CTCCAGGTGAAGGGAACAGCAGAGCAAAGGCCTTGAGATGGGACTTCCTTCCCCTCACTCCTTT
GCCCCAAGACCTGCAGAGCACAGGGCCTTGATTGACAGGCTGTGGAGGTGGGCCTTACCCTGTG
GGCAGTGGGGAGCCATACACGGTGACAGGCCAGGGTGGGGACACTGGATGGGAACCAGGGTTTG
ACTCTTTCTGAGTGAGTGCTGTGCAGACGTGAGGTCATCTATTTTTTCCAGTACCCGCTAGGTA
GCAGGCACTATTATTGTTCCCATTTTGCAGACAAGAAACTGGGGCTCAAAGAAGAGAAACACCA
CATACTAAGTTACACAGCTTGTCTGTGGTCGAGTCAAGACTCAAACCCATGTCGGCATCAGGGC
CAAGCCCGAGCTCTAAATGGTTCTCAGCCTCGCCAGCTGACCCAAGGCAGTTTCCAGTGGCCTA
AGGCAAAACTGAGGAGAATAAGGACGGGGTGGCGACTCTTTCATAAAGCTAAATTTATTCCACT
TAAAGGACTGTCTTTTAGTATGAGATGATGTCCTTTCTTTTATGAAATGATGGGCTGGGAAAAA
AATGTCCTTGCTTGGCAAAATAAAAAGTTGACAACCTTATGCAGTCCCCAAAGTGTTTTTGGAA
ATTTTACCGGTGGTGCGTGAAATCCCTGAGTCTGGGCGCCATCCCGGCTCCTCCCCACTGAGCT
GTGGAGCCAGAAGAGGGAGGGGTCTGGTAGGGACAGAGGGATCAGGGGACAGAGGAGCCTCAGG
TCCCGCACTTTGCAGTGAGGACCAACCCAAGGCTCTTTCCTAAGATTGGGTTGGGCGGGTGGGG
GGGTGGGGGGCAGATCAGCCAGAATCAAGGGCTCACCCCTCAGTTAAAGGAGCACTGGACCGGG
AGGCAGCAGGCTTGGGCTCAGCTCCTGCCTCTGCTTCCTGGAGGCTGTGTGATGCCAGGCAGGT
GACCCAACCTCTCTGAGGCACCAGCTTCCTTATACATAGAATGACAATTGCCATCTGTCCCTGC
TCACCTCACGGGGCTGCTTCCTATAGCAAGTGCTTGGTAAACTGCAAAGTGCTGTGAAGAACAG
GGGGGTTGTGGTGGTTTTACCTTTTTTTTTTTTTTTTTTCCTTTTGAGACAGAGTCTTACTCTT
TCACCCAGGCTGGAAGGCAGTGGTGTGATCATGGTTCACTGCAGCCTTGAACTCCTGGGCTCAA
GCGATCCTCCTGCCTCAGCCTTTCATGTAGCTGAGACCGCAGGTGCACGTCACGGCGCTAGGCT
AATTTTTAAATTTTTTGTAGAGAGGGGATCTTGTTATGTTGCCCAGGCTGGTTTCGAATTCCTG
TGCTCAAGCAATTTTCCCACCCTGGCCTCCCAAAGTGCTGAGATTACGGACATGAGCCACTGCA
CCCAGCCCTGGTTTTACTGTTCTTATCTTGCCATCAGAGGATCCCACTTCTCTAGGGTCCTGGG
CCCCTCATCTCACTTTCACTTTTCACTTGCACTGCTTTGAGCCCTGAGGAAGAGACATATGGGG
GAGCGGGGCAGGTGGAATGACACAGGCCCTGTAAAAGCAGGGTGGCCACACTAGCAATTCCTGT
CCTCAGGCTCCCCTGGAGGACAGCTCAGAGATGCCCAGGCTGGAGACCTGGGGAACTCTAGGAA
AAGAATGCACGAAGCTGGAGCCAGGCTAGAGTTCTAAGCACAGACTTTGTCACTGAGTAATAAG
ATTCATAGTCTAATCTCTACCAGGTATTGTGCTAAATATTTTTCACACTGCTTCATTTAATCCT
TCCAAGAGCCCTATGAGGTAGGTCCTATCATTATCCCCATTGGATGGAAGATGACACTGAAGCT
TAAAGGTGAAACACTGCCCTTGTCACACAGCCAGGAAGTGGTGAATGCCCAAGAGGACGGGAGT
TTTCTGGGCTCAGCTCAGACAACTGAGTGCCTGGAGAGGCCGAGTGTGCAACGATTCTGTGACA
TGAGAGGGTGGGAACGGTTTGCAGGACTGTGGTGGGGGGCCGAGGGGGTAATCAGTGCACATCA
GGGGCACCGGGTTGGGGTCGGAAGGCAAGAGACACGATCCTGCTGGCTAGCTTCTAGCAGTCAC
CTCCCTCTCTGAGTCTCAGCCTCCCTCTCTGCTCTGGCTCTCAATAATAACAAAGATAATACTA
GCAGGACCTTACTGAGTGCTTACCGTGGACCAGATGCTGCTCTGAGAGCTTGCCACGCCTTCTA
TCACTTTTTCCTCTCAATGACACTATGAGGTAGGTACTGTTATTATGCCTACTTTTCAGATGAG
GAAGTTGAGGCACAAAGAGGTTACGTGACTTGCCTGGGTACATGTAGCAGGGCCAGGATTCTAA
CCACAAGAGTCTAGCTCTATAGGCTGTGATCTTACCCACCTAACTCACCCGGACTATGTCTGCC
TGCTTCTAATGGGGGGTGATATTAGGAGAAGTTGAGCCTTAAAGTTCAAGGTCCTGGGCCGGGC
GCTGTGGCTCACGCCTGTAATCCCAGCACTTTGGGAGGCAGAGGCAGGCAGACCACCTGAGGTC
AGGAGTTCGAGACTAGCCTGGCCAACATGGTGAAACCCCGTCTCTACTGAAAATACAAAAAATT
ATCTGGGTGTGATGGTGCGCACCTATAGTCCCAGCTACTTGGGAGGTTGAGGCATCAGAATCGC
TTAAACCCGGGAGGTAGAGGTTGCAGTGAGCCGAGATCGTGCCACTGCACTCCAGCCTGGATGA
CAGAGTGAGACTCCATCAAATAAATAAATAAATAAATAAATAAATAAATAAATACAGTTCAAGG
TCCTGCTCCCACCCCGATTCTACCCTGGGGACTCATTCCTTCCCTCTCTCGCCCTTTCATTTGA
GAAACCTTCACTTCTAAATGAAGTTCAGCTGCTTATGCCAGTCTCCCCGGGCCAGGCCCTGTGC
CAGACTCTGGGGACAGAGCAATGAATCAGACCTTCCTCTGCTCCCTGGGGGAGATGGGCTGGGA
AGCTGGCAGTTACCAGGCAGGGTGATAAGTGCCACTGTCCAGGCAGCACGGAGTCCAGTGGGAG
CCCAGCACAGAGGGCCAAGCCCTGCCCCAATCTGGGCATGGCTGGGAAGGCTTCCCAGAGGAAG
AGGCTTAAGTTGAGACTGAAAGATGAGTAGAAGTAGGAGTTAGCTAGGTGAAAAGGAGCAAGGA
ACAGTGTTTCAGGCAGAGAGAATAGCACAGGCAGAGACCCAGAAGTGAGGCTGAACAGGGCATT
TGCAAAGAACAGCAAGAAGTCAGAAGAGCGGGATCCTAGAGGAGGCAAGAGGTATCTGGAGAAG
AGGCTGGGATGCAACAGGGCCAGGCTCGGCAGTGAGGACACCGGGCTGGGGAGCTGCAGCTTCC
TGAGAGGGTGATGGGAAGGAAGTGATGTCAGGAGTAACATGGCAGATTCGTGTTTTAGAAACTC
CTTTCTGGGACTGGCGAGGAGGATGCTGGAGGGGTAAGGACTCTGGCAAGGAGCCCTGGGGATG
CTACCAACTTGTCCCCCTTTTTCCTTTACCTTCCCCCCAGGGTCCAGCCTCCTGAGTGCCTCCT
CCAGAGCTATTGACCAACCCTGCTCAGGGCCTGGCTCAACCTGACCCGGAGCCCTAGGACTTTG
CCTATAGGGAAAGTTCAGCTGCTTATGCAAACTCTGGCTTGGGGGGAGATGTGTGTGGTTGAGC
TGGGAGCTGGTGGTGTGTGGGGGTGGCCTTGGAAGCAGAGAGGGTGGGTGCAGGCGCCGAGGCT
GTCTGGAAAGGGAGTCTGGGGATATAAGCTTGGGGTCCATGGGGGTCCCTAGAATAGTGGCCTT
ATAATGGCTCCAAGAGGGTACACTGGGATTTGGCCCAGATAATGGAGGGACACCCTGGCGAAGG
GGCAGAGTCCTGGCAGTTCAGCCAGCCCCAGCTCTGTCTTTTTCTTGAAGCTCAACAGAGTATG
GAAATCATTGGTCTCACCCAGCCCCCTCATTCTTCTGAGGGGGAAACAGGCTCAGAACAGGGCA
GGGACTAAACTCAGGTCAGCAGTAGAGCCCAGACCAGAACCCAGGCTCAGTCCAAGCATCTTAG
TTGTCTCCAATGATGATAAATAACAGTGAAAACCTTTCTGCAGAGCTTCCCAGGGGCTGGCCTG
GGCTTAGGCACCCTCTGGATGGTCTTGGGCAGACGTTTTTCACATCTCGAGCCTTAGCTCGAGG
TCCAAAATACTACCTGTACTGGGAAGGAGGTCCTCTCTCCCACTGACAGTCCACAAAGCCGTTT
TAGGCATGCTGGACCCATTGTGGGCTTTACTTATTGTTTGTTCAATGAATCCCACTGAATTCAT
GTAACAACCCTGGAGGAAGATACTATTATGCTATTTTGTGCTGAGGCTCAGAAAGGAGAAGTGA
CTTGCCCAAGGTCATTCAGCTGAGAAGTTGCTGACTTAGGGCTGGACTCCAGGGGTCCAGATGC
CTGTCTAGGATCCCTGATGGACACAGCCCCATTTTTGAGCCTGCGCTCTCAGCGAAAATCAGTT
GGTGTCTCAACGAGACCCATCGTCAGCTGGGCAGGGTGTGTGTATATATGTGGAAGCTGGGCTG
GGCTGCTGGCTGGAACTCAAGCCACACCAACCCCTGTGCCCCTGTGTTTGGACCTGGCTGCAGC
TGCTGCCCGTGGGGGAAGGGGACCTTGCCACTTGCCCTCCTGCATCAAGGGTTTTTTCTCCTTG
GCCTGGCTGCCTGGGCAAAGGAGCTGGCTGTGGCATGACCATGTGGGCACCTGCCAGCCCCTCC
CTTGTTGGGGTCTGGTCATCTAATGCCCTTCACATGACAGGCATGGGGTGGGCCCTGGCTGGGG
AGAGGGATTTGCACTCTCCTAATCCAAGGGCTGCAGCCCTCCCTCTGTCTTGGCCAGGGCCGTG
GGAGAGGTTTGTCCTTGCCCCATGCCCAGCAGCCCCAGCTCAGATCCCTGGCATACACCCAGTT
CCTCTGCTTGGGGCCTTCCCCTGTCCTGCTTCTGCTCCCCACCCTCCTACTCTGTGAGCAGAAT
CAGGAGCTAGGGGGCAATGCTGGGGCATTCAGGACTGATGGAGGTGCAGTGCTACATAGCATGT
GGGACTGAAGGATGGGGGCGATGTCAGGGAGGTTTAGGGCACATGTTGGGGCAAAGTTGAGGGA
AGATGATTGGGGTGCAGAGCAGGAGTGTTTGGGGCTCATCCTGGGCCACCCCTTCCTGGAAAAA
TTTGGCTACAAGTCAGGGAGGCAAATGTGTGGGCCATGCCCCCCCCCTCGCTAGGCAGTAAACA
CTGCCGTCGCCTGGGAGACAAGAAAACATCTTGGTCCCCCCACGGTTGTTTATACCAGGACTCC
CTCTCCTTCTCTGGCCTGGTCTGGCCTCTCCACTCCTGAGCTGGGCTACCTGAGTTGAGGGTCC
AGCCCCCGGTCATGGCAGGAGGCCCTGGCAGCACGCCCACTGGCCCCCACCAGGGACTGAGCAC
TCACCACCTCCTGGTGGCCTGTCCCATTGGAGGCCCTAGGTCTTATTGATCAGAAACGTGCTCC
CAAGCCTTGGCCTCCACGAGGGAGGCCTGGGACTGTGCCCTGGATTTGCTCCAACCCACTGCCT
GAATGCAGTCACTGGCCTCCCGGTAACCTCAGCTTCCCATGTTCAGTAGCAGGCAGGATCTAGA
TTAGAGCTTTTCAAACTGTTTCAAGTCATAGATCTGGTTCTTTGAATGAGACCTGATACAGAAG
CACAATATATAAAACAGATCAAAGTAGGGCTACTCTGGGTGGAGTCACGGGTTGGGGCCCAGAA
TTCTACCCACTCTATGCCAGCCTATATGGCCCTGGCATATCAACCCAGAGCCTGTACATGGTCC
TCCTTGCTCTAGAACACATTCCAGGTAACCCTGGAATTCCCTGGCCAAATACTTATGCCTACTT
CCAGGGTCTGCACAGGCCCTTCTCCCAGGGGCTGTCAGAGCAGCCCTAGGCTCAAGGACGGCTG
TTTGTAGTGGGTATAGCAAAAGCCCGGAAGAGCAGGCTGAGGCACCCCCAGGAGTGTACCCAAG
GCCCCTCCCAGGGAGGGATGGGCCCAGTGTGGGAAGAGAATCTGAGGATCGGAGGGTAGCAAGG
GGCTGGCTGTCCCTTCCCTGCTGGATTCCAGCATGGGATTCCAAGGCATCCAAGAACTAAAATT
CAAACCTGGTCTTCTAGGTCATTATGAAGGTATATGTGTCAGGAAGGATAGAGCAAATTTTATT
TAAAAGTTTGTTAACCTGATTGAAACTTTTAAATATTTAGACTTATGGTATGTGGGTCTCCATT
TATACTCTCTCCCCAGTCCTGCCAGAGTTAGGGGTGAGCTTGCATCAATCCTCCTTCCACCCAC
CCACCTACCTCTGCAGCAACCCCAAGACAGCCTTACCCAGAACTCTCAGGCTTCACAGGACAGT
GTGGAAGTCCCACTCGCTGAAACTCAAGGCCCTGGTGACTGCATCTTTCTGTTCACTCCCCAGA
TACTGTCTGAGCATCAGCTGGGGCCAGCCTGGGGGTTGTGGAGGAGCCAGACTGGCCTTGCCCT
AGAGGGATGGATCTCAGTGAGGAGTAAGCGGGTAGGAACACTCTCCCTCTTGTAGAATAGTGTG
AGAAGGGGTGCCTGAGGGGGCCCACAGGGGCCCTGGAATCCTAACGAGGGGCTAAAGAAGCTCC
TCTATTCAAAGCCGATCATGGATGGATGAGGTGGAAGAGAGACAGCCCAAGAAGGGGAGACAGT
ACAAAAGCCACCAAGGTGGAAATGTACAGGGAGTGGAGAAGTCGAGTGTCGCTGGTGCTTAGGA
TGTGTGTACAGCCTCAAACGCTGGTCTAAGGAGCTTGGGTTTTCTATTTTAACAGTAAGAAGCT
GTTGTAGGATTGTAAATGGGGTAGGGGCTGAGGAAGAGGCATTCAGACTGCAAGGTAAGTGGAC
TGGAGGTGGGGATAGGGGGCTGGAATTGTGGTGGTCAGTGAGAGGCTGGCTGGGGGGAAGGGGA
ATGGGGGTGAGTCCTGGGCTGACCAGGAATGGGACTTGAGGGGCTAGGAAGAAAGAGGGGATCT
TAACTCTGCTTTCTGGGACCACATCTCCTATCTCCTCCTCCTTTGTTCTGTCTCAGTTTTTGTC
TTCTTCTCCTGCTATTCCACTGCCCTAGGTCTCCAGGCTGGGGAGAGGGGCACTCACCCTAAAC
CCCCTCTCCAAAGCAGGCCACTTCACACAAGTGGCCAGGCAAGAGGCCGCAGTTGTATAAGAGG
CTTTTCTCAGCTGGCTGGACTACTCATTCGTTCATTTATTCATTCATTCACTCAACATTTATTG
AGAACCTACAATGCACCAGGCCCTGCACTAGGTTTTGGAGAGAAAGTAGGAACAAGATCAAGTA
CCTGCATTCATGGAGCTTACATTGAGGGGGGCGGGAGAACCAGGAACTGTATCTATCCCCGGTG
TCTACCATCTGGCACCTGTGCCTGGGCTGGATGCCTGGGAACAGGGACTCTTCCCATTACCTAC
TCATTTGTTCCAGCTGCCCTGTGTCAGCACTCACCTCTCTGGACAATGACAGAGGAGAAGGGGT
CCCATGCCCAGGATGCCAGCCCCAGGGAGCACCTGTCTCTGTAAGAACAGCCTGGCCCTCGGGG
CCAGGACCTGGGCTCAGGCCTAGTTCTTCCTACTCACTCCTGACTTTGAGCCCTTTGCAAGGAC
CCGACTTTGATTTCTTTATCTGTCAGGTGGGATTTGGGGAGGTCCTGAGGAGGCGAGGGGACCA
GAATACTGGACCTCAGCTCCAGTATTCTCCCTTGGTGGGCAACTCCTTTGGCTATGATGGGAGG
GGGAAGGGGGCTTGGGACTCAATATTATCATCATTACCCATACAAGATAGTAATAGTTGCTACC
TATTGTGTAATTTATTCCGCACTATCTGTATGGTCGGTGTGTCTTCTCTCCCCATTTTCCGGAT
GAGGAAACTGAGTCTCCGTAAGGTGAAATGACTTACTTGAGGTCACACGTGAAACTACCTACTC
CAAGAGCCCTTTAGGATGGTGCTGTGAGGTGGAAGGGCGAGGCCGAGGCGGCGGTGGCGTCGCG
TCCCCCCTCTCCGCCCCTGGCCTCGGGGACGCGGCGGCACCTTCACACTTTCCGCTTGGCCCGC
TGCTCGCCTTTCAGGCCGCCGCGTTTTCAATTGTTAATTTGGAAACGGAAAAAGTAGCCGGCCG
GGCGGGGGAGGCCGCGGGGAGGTAATCGGAGCCGCAGCACCGCCCCGGCCCCTCCCGCTCTCGG
GGCCTGGCATTGGCAAAGCCCCGCCCCTCCCAGGGACGCAAGAGCCCCGCCCACCATTCCCCGC
CCCCGGCATGCCCCGCCCATTTCCGTAAGCCCCGCCCTCGCCGAGGCGGGCTGGGGGCACCTGG
GAGTGCCGAGCAGTGGCGCCCCTGCTGAGGAGAAGGAGCCCGGGCGGGGGAAGGGGGCCGAGGG
CTGTGCTGGGGGAGGAGTCCCCCAGGAGCCCTCCCCGCAGGAACCCGTACCTCTCCCGAGAAGT
CGCGCAGGGTGTCTGCTTCCCTTGTCCCTACGTGAACCCAAGCCTTTCTGCAACCCCTCTGTAG
GTTCACCCAGGGGAGACCCCTGCCTCCAGACTGCCGGACCAGGGATCATTGGTCCCATTAGAAA
GACGAAGAAACTGAGTCCTGAGAGGGACAGTTAACTTGCTAAAAGTCACACAGCCAAGGCAGAG
CAGAGCTGAATTCAAGCCCATTCTTTTGAGTGAGATACATTTAGGGACTGTCTGCCTCATTTAG
AAGAGGAGGGGGCATCAGGACCAGAGATACAATTTGTCCCCTTCCCCCTATCTCCAGCACGGTT
TCTGTGGTTAGTAAGTTACTGGAAGTTCTGCTTATCATTATTCAATTATGGGAGGGTTTTGGAG
GTAGAAGGAGGTCCCTCTAAACTTTCCATGTAAATGAAATGTAAAAGACCCTCTTCTGGGGACT
CCCAGAACACAGAAGTCCCTGAGGACTGGCAGCTTCAGGAGCCGTCCACGGCCTTGCTTCCCAT
TAGGGAAGCTGGAATTGCCAGTCCCAGATGGATCAGCAAAAAATAGGGCCCTCAGAGACTCCCA
CCCAATCCCTTCCTCCCACTGAGGAATAAGCTGAGACTCAGACACAGTTTCCCAAGTCTTCAGC
TGACTGGTGGCGGAGTCAGGAGACAGAACCCACCGACCCAGCTTTCTAGCTGTGTCCACGCCCT
TCTGCTTCTCCCTCTGCTGCCCCCCAATCCTGACACCTCTCTCACAGCCACCACTCAGGATCCT
GCCTCCATCAGCTGGGCCTGCAGGCTGGGCAGCCTTTCTCTTTACAGGCGGAGGAAACTAAGAA
GAGTATCTGCAGATGTCCTCCCAGCTTCCAGTTAAAGACCCTCCCAACCACCCTTCCACCTTAG
CTGTGGGTCAGGCCCAGAGAAGCTGCGCTCCTGGCCTCGAGTCACACAGCAAGCCTTTTCCAGC
TTGCAAAGCCCACTGGATTCAGTCTGGCTGGTCCTGGATTTGGGGTTCCCACTGGGAGCATGGC
TCTCAGGGCAGCTGTAACCAGATGCCCCAGGTGTGGCCTGTGAGTTTGCATGCTGCTTTGCTTA
TTAGTAACAAAGGCATTGAAACCCCTCTTCCTCATTGTGTCCCCAAACCACCCACTGGGTGGGG
TCCTCAATGGCTGAGTCTACAGAACTCCTGGGCTGGGCTCAGTCTTGGCACCTGAGCTCCCCAG
GGAACTCTGCCCTCCACGGTGCTCCTCTTCGACCCTGTCAGCTCTTGGTGCTCTCTCTGCCCTG
GAAGTACTGAGAACCCAGTGGCAAGGACTAACTCTTTCCCTTGTCTTGGACTCTTCCCTCCCTC
TCCCTCCCCCAATATAGTGCACAGGTTCTGCTGACTCAGAGGACAGAAGGCTCCTTTGAGGTTA
CCCTGACCCCTGCCAGGTTCAAGTAGCTGGGAAACTGGGCAATGCTTGAACCCACTGCCACAAA
GTCACATGCAGCAGGAAGGGCAGGGGCAGGTCCCATCTTGGCCCCAGGATGAGGGAATTTTTGA
GGTGGGAGTGAGAGGCAGGTGCCTGAGTCCTTCTACAGACAGAGGAGAAATCCAAAGAACATTG
CATTAGGAATCAGGAAATCTGGGTGCCAGGCCCAACTCTGTCTCTGATCTACTGTATGATCACA
GGTAAGCCAGGCCCCTCTCTGGGACTCAGTTTCCCCATCTATAAATGAGAAGGTTGGTTAATCT
CTAAGGGACCTTTCAGTGCCCAGATATAGCCAGTATCTGGTCATATCTTGCCACTTCCGCTTCC
CCTCATACAAGTCACCATCATCAGCTGGTAGTGGCTCATGCCTGTAAGCCCAGCACTTTGGGAG
ATGGAGGCAGGAGAATTGTTTGAGCCCAGGAGTTCGAGACGAGCCCGGGCAACATGGGGAGACC
CTGTCTCTACAAAAAATAAATATAAATTAGTCGGGCATGGTGGCACCCACCTGTGGCCCCAGCT
ACTCAGGGGGCTGAGGCTGGCGGATTGTTTGAGCCCAGGAGGTTGAGGCTGCAGTGAGCCGTGA
TTGCACCACTGCACTCCAGCCTGGGTAACAGAGTGAGACCCTGTCTCAAAAAAAAAAAAAAAAA
AAAGTCACACAGGGTGAAGTGGCATGTGCCTGTAGTCTCAGCTACTCAGGCAGCTGAGGTGGGA
GGATCATTTGAGCCCAGGAGTTCTGGGCTGTGGTGAGCTATTCCAATCAGGTGTCCACACTAAG
TTTGGCATAAATATGGTGACCTCCTGGGAACAGAGGACCATCAAGTTGCCTAGGGAGGGGTGAA
CTGGCCCAGGTCAGAAACGGAGCAGGTCAAAACTCCCGTGTTGACCAGTAGTGGGATCACACCT
GTGAATAGCCACTACAATCCAGCCTGGGCAACATAACAAGACCCCATCTCTTAAAAATAAAAAT
AAACAAAACAAACAAACAAACAAAAAGTCACTGGCATCTCTTGCCAGGATTATACTTTCCAACT
CACAATGGGGCAAACGTCATTGCTCCCTCCTCCCCTCTCCCCCACCATAGTCCGTTCACTCAGC
AGCCTGTGTGATCTTTTTAAACATAAGGAAGATTGAGCTACTCCCTGCTCAAACTCCCCATCTC
ACTCAGAGTAAAAGCCAAGATCCTTACCATAGCCAGTAGATGGTCCGCTTTCCACCTCCCCTGC
CTCTCTGCCCTCACCCCCCACCACTCTCCCCTTGCTCATTCCACTCTGGGATCACTCCTTTCCT
GGTAGTTCTGGAACACAGAAGGCACCCTTCTGGCTCAGATGTGTTGGATTTGTTTTTCCCTCTG
GAGGTGGGATGGGGGTTAAGGGAGCATGAGGTGGGGGCTCTGACCTAAGCCTGCCAGTCTGAGC
TCATGCTCAGACACAGCATGAGTAAAGGTGGGGAGGGGAAAAGAGCGCGTTTGCCATTGTCAGG
GACAGAGCTATGATAATGCAGTGCCAAGTGTCAGGTTGGGTACCGGCTAGTGAAGAGCAAGTTG
GCCTGGTGGGTGGGGCTTTCCTTGCAATCATGCTAAGGGGTTTGGGCCTGGGCCCACAACTCAG
TGGATTTCACACTTTTTAAAAAAACAGCAAAGCTTTTTGTTCACATGAAATTTTTTTTTTTTTT
TAAGATGGAGTCTTGCTCTGTCGCCCAGGCTGGAGTGCAGTGGCGCAATCTCGGCTCACTGCAG
GCTCCGCCTCCTGGGTTCACGCCATTCTTCTGCCTCAGCCTCTGGAGTAGCTGGGACTACAGGC
GCCCACCACCACGCCTGGCTAAGTTTTTGTATTTTTAGTAGAGATGGGGTTTCACCGTGTTAGC
CAGGATGGTCTCGATCTCCTGACCTCGTGATCCGCCTGCCTCAGCCTCCCAAAGTGCTGGGATT
ACAGGCGTGAGCCACCGCGCCCGGCCTCACATGAAATTTTTACGCCAAAGCCAGCATGTAAAAC
TGAGCCACCCGAATCTGCACGGAGCTGCTCAGTGGAACCGAGGGAAGGTTTCCACTAGCCATCT
TGCTTGAGTTTCTCAAATCCTGGCTGCCTAAATTCCACCCCATGTTTCCCCTTCCTTTCAATGG
CATCCTGCTTGGGAGGGCCCACACCTCCCTGTTTGAGAAGTCAGCTCTGTCTGGCCAGTCCCAA
CAGATGGATGCCCCTGTGTCCTCTTTGTTCCACTCTGGCTCTTGTCAACACATTGCTCAGCCCA
CTGACCTGGTGCAACAGGTAGGCAGGCTGGCACGGCAGAAGGAAGCAGACAGAACAAGTCGGAA
GGTTTGGGGCTGGTGCAGGTGGGAGAGGAGGAAGCTATTCCTCATCAAACACCTTGTCTAGAAG
ACACTGCCTTTCATGTCCCGTGGGACAGAAACCATTCAAGCTGGCTCAATAAAAACTGGCTTCT
GTACTATTACATTTGGGATAAAGAAAAAGAAAAATTGGCTTCTGGAGGTAGAGGACAGATATGG
TAAGGAAAGAGAATTCTTTAGAGGCTTCAAGGCAGAAAGCACATTTTGGATTCATGGGATAGAG
AGCAGGGGGATGAGGTTCCAGCGTAGTGCAGTCTCATGGCCCATCTCTCTGCACCTCTGCCAAC
TTTCCTCTGTGGTTCTTAGTTCCATTCCTGCGAAAGACAATCCGATTGGCTCAGCCCAACCTAT
GGGTGGGCCCTTCTTGGGTCAGTCTCCAGCTCTCCATGAATCTGTGGCGGCCAGGAGAGTGCTG
TGGAGCCAACCGCAGGGCTGTCATGTGTCGCTCTGCAGTCTGGGCGCCACAAAGGGGCCGGCTG
AGAGGGCTGAGGGGTGCTGGAATCCAGCCTATGCCCAGGGGTGGGGCAGCCCAGGGGAAGGGAT
GCCTGTGCTAATGGGTCCGTCTAGAGGGGTCAAGTTTTTATAACTTGTCAGGCTAACGGGAATG
CTTTTTTTCTAAGCCATCCTCCAAGAGGGGGCATTTTTTTCATAGTTTGTTCTATGTGCTGGCG
GCTGCTGTCCCTCCAGGGACAGCCTCAGGGAAACTCAGCTGAGGGCCAACTCTCTCCTTTCTAC
CCCACTGCTGGAGAGTCACAAGGCGGAAGGGACTTTGGCCATTACTGAGTAGTGCTCCACCTAC
ATGTTGCAGACAGGGAAACTGAGGCTCCATCAGGGCCCCATTGCAATTTCTAAGCCCCAGCGCA
AGCCTCTGGCCCAGAGAAGCAGCAGCGGCAGTGGCGGCGGCCCTGGTGTGGGGCCCTGGGTCAC
GTGCTCTTGCTCCCTTTGCCATATTTAGAATCGCTGGATCAGGGGAAACTTCTCCTCCCAGCCC
CTGAGGATTCTTGGAAAGAGCCTCGCTCTGAGGCCTGGCTTACTGCCCCTCCCCTTATTCCCCT
GCAGGAGAAAGTAAGTGCCTGCTCTCTCTAGAAGAGTCTGGGGGCCTCCAGGCTGCCCCCCTTG
TCAGGAAGTGCCTGTCAAGTCTAGCTGAAGTCCCTTCTTCGGTATCAGCTCCGGTTTGGGGTTT
AAGGAGCCCCTTTCTTCCAGAAGGTGATGGTGGCCATAGCTTCTGCCCTCTAGGGACCTTGGCT
GAATGGAGGACAGACTGGCCTCAGGGTCCTCCAGCTGAGGGGAGACAGAGCCCTGCCTTTGGGC
CCTGGTCTAGGGGGCAAGGAAAGAGTCACGCGCTCCCCAGGCCAGATGCACAAATCACTTGAGC
TTGGGGGTTGGAGACCAGCCTGGGAAAATAGCAAGACCTTGTCTCTACTAAAAATCAAAAAAAA
TTAGCCAGGTGTGGTGGTCCCGGCTACTCAGGAGGCTGAGGCAGGCAGATTTCTTGAGCCTTGG
AGAGTGATGCTGCAGTGAGCTATGATTGCACCACTGCATTCCAACACAGGCAACAGAGCAAGAC
CCTGTCTCAAAAGAAAAGAAATCAGGGAAACTGCTGGTGGCTGGGCAGTCCCCAGACACTGGCA
GGGCTCAGTCTGGGAGGGACGCTGGAAAGACGAGGGAAGGAAGGAGGCTCTGGAGGAAGGAGGG
GGCAAGGCCTGGATCTGTATTTACCTTGGCCTTGGTGGATACTTGGGGGTGGGGAGCGGTCCTC
TTCTCACACAGGCTGCTTCCTAGTGGATAATCAAGCCTTAAAAACAAACAGCAACAAAAAACCT
AAATAAAACCCTGAAATGCATGTGTTGAGTGCCTGTGATGTTCCAGGTCTGAAGAGCCATCCTC
GCTGCTGAGGATAATGGTCCTGTGCTCCCATGGGACCCCTCTCCAGGCGTGGTCTTTTCTCCCA
CCTTCCTCTCCCAGCTCCATACCTCCTCCATCTCTTCAGATCCCCCACCCCACCCTCCTGCCCA
CTTCTGAGAGTTGGGCCTTGCCTCTTCCCAGAGCAGTCCCCAGTGAGTAGGGGCTGTCGGCCTC
CTCCCTTCCCACAGCCTCCTCCCACCTACATGCCTTCCTGTCCCTGTGGAAGCTGGAGGGATCC
TTCCTACTCCACACCAGCTCCTCCCTGGCTCTGGATCCCAGCCTCCCATCTTCCTTGGGGACTT
CATTCCATTGACTACTCACTCTGTCTCCCCGCATCTTCAACTTCTCCTTCTTGATCTCTTGGCT
CCTTCCACTCTGGCTCTCTCATCTTAAAAGAAAAGAAAAAGATTCCCTATACCCTGCATCCTTC
TCCAGCTACGATCTCTGATCTCTTCTTTGCAGTCAAACATCTTCAAATTGTTAAACCTACACTT
ACTGTCTCTACCTCCTCACTGCCCCTCCCCTTTCTCACTTTTTCTGGGCCTGTTCTGGTCTTCC
TCTCCTTCTGAGAGAAGGCTGCCCTTGCCAAGGGACCCAGTGACCAATGTGTCACCAAATCCAG
AGGACATTTTTCACGCCTTATCTCACTTGACCTCTTGGCAGCATTTGACACTGTTGTCTACTCC
CGCTTTGAAACATGTTCTTCACGTGGCCTCTCTGACACTTCTCTCTTCTGCTTTCCCTCCCAGC
TCTCTGGCTATCCCTCCCAGTCTGCGTAGAGGGCCTCTGTTCCTGCTGCACCGCTGGGGCTCCA
TCGGGTCCTCCTCTAACTTTGCTACAAGCTCTCTGGACTGTCTCAGCTGCTCCTGTGACCTCAG
TCACCACCTCTACCCTGATGTCCCCCAGATCTGCATTTCCAGCCGGGTCTCTCTCTTCATACCC
ATGGAGTGTTTGCCCAGTGCCCATAGCCCTCTCTCTCTCTCAACATGCCCACCACCCAACACCT
GCTCCTCCAGTGGGGTCTCCCCCGTCCCATGAAGGCACTCATGGCACCTTCATCCATCCAATGC
CCAAACCAGAAATAGAGAAGTTGTTCTCCTTCCTAGTTCCCGTTTCCAGTTAATCACCGAGCCC
TACTTCCTGCGGTGTTTTCCAACCCATCCACTCCTCTTCCCTGCTGCCACCACCCTGGGCCAGG
CCACAGTCATCTGTCGTCTGAGCTATTGTGGAGCTTTCTTGGTCTTTCTGCTGAGTCTTATCTC
CTTTGATCTATTCTGTGTACTTCCCCAAGTGGTCCTGAGATGCAAATTGGACTGTGTCATTTCC
TCTCTTAGAACTCCACAATTGGCCAGGTGCGGTGGCTCACGCCTGTAATCCCAGCACTTTGGGA
GGCCGAGGCAGGTGGATCACTTGAGGTCAGGAGTTCAAGACCAGCCTGGCCAACATGGTGAAAC
CCCGTCTCTACTAAAAATACAAAACTTAGCCGGGCGTGGTGGTAGGCACCTGTAATCCCAGCTA
CTCGGGAGGCTGAGGCACGAGAATCTCTTGAACCCAGGAGGCGGAGGTTGCAGTGAGCCAACAC
CACTGCACTCCAGCCTGGGCGACAGAGTGAGACTCCGTCTCAAAAAAAAAAAGAACTCTACAAT
GACTCCCCATTGCCTTTGGGATCATGTTCACAGTGCCTGAGAATGCTACTCAAGGCCCCTGGGT
CTGGCCCTCTCTGGTTTTTCCACTGCCCCCTTCACTTTAGGCCACACCATCCAGAACTGCTGGC
CGTTCTGGGCTGCCTCGACGGCCACATTCCCACTGCTTGCACATCCTGCTTCCTCTGCCTGGGG
CACCCCGACCCCTGTCCAGTTACCTCCTCCAAGAAGTCTTCACTGATCCCTCCACTTTGGTTGT
GCGGAAGGCTTCTTGGGACTCTCCCCGACCCCTTGAGTACACTGTCATGGCCTCGTCCATCCCG
TCACCACTGTGTCATGTTCTGTGTTGTTAATTCCTTGGGACCTGGATTATTCATTTCTGGTTTT
ACAATAAACCTGGCTGAGTGTAGGTGTCTGAAGTGAATATAGCCCAGAGAGGTCAGGTGACAAC
CTCTTTTCTGCTGTGAGATGCCCTGCCATCTTTCCTACCCCCATCATATCCCCATCTCTCTGGC
CTGGCCTCCCAGCAGCTGTCCCTCCAGGGACATGTTGGACATGTCGAGAGAGAGAGCCGTGGAC
ACTGGGCAAACGCCCAGTGGGTATGAAGAGAGAGACCTCAGCTGGATCCAGGGACATCCCCAGA
GAATCTGGAGCTGAGCTGATGGCCGATCCTCTCCACCCCACTGCTGGAGAGTTACCAAGCTACA
GGGACCTTAACCATCACTGAGTTGTCCCCGACCTTTGTGTGTGTTTCTGACAGGGAAATTGAGG
CCCAGAGCATTAGTAGGGCCCGGTGGTCCAGGTGGTCTTGGGCTCAGGTCCTGCGCTTATGACT
TTGAACAAGTCATACCTTTCTTTTCTCATTTGAATAACAGCTTTATTGAGATATAATTCACATA
CAGTAAAATTCCTCCTTTTAAAGTGTTCAGTTCAGAGGATTTTAGTGTATTCACAGAGCTCTGC
AGTCACCACAGTATACGCCTCTGAACCGCCCTCTCCTTATCAGGGTGATGATGGTACGGTTCCA
TGGGGTCACTGTGAGAACTCATTAAGAACATGCATTTAGAGGGCTTGGCACAGTGTCTGGCCAA
TAGGAAGCCTTTGGCCAGCAGGAGCAGTTATGGTTATTACTTCAGGTCACCACATGAGAGCCAC
CTCCCCAGCAGAGCTTTCCCCCTCCTGCCCTGTGGCCGCTTCTGGACAGTGACTTGAGGAGGTA
TAAATGTGGAGAAGGCGGTGATTGCAGTTGTTTAAAAGCCCTGAGCTGGCTAATAATTGAGTCA
GCTGCAACTGCTCCCATCACCTAAGCCAGTGATTCTCAAAGTGTGGTCCCCATAACAGCAGCAA
TAGTATCACGAGGGACCTTTGTAGAAAGGCATATTTTCCACTTCAAACTTACTGAATCAGACAC
TGTAGGAATAGGTCCCAGAAATCAGTATTTTAACAAGCCCTCCAGGTGACTCTGACACCCACCG
TAGTTTGAAAACCACAAATCTAAGCAAACCATTCACTTTTTCATCTCTAATCCTCTTGCTCCCT
CCTGAACGAGTGGGATAGTCATGGGATGGGGAGATGGGGGAAATGGAGAGGAAAGGTTGGATAA
GCCTTGTGATCAGACCAGCTGGGGAGAGCCAGCCAGGCAACACCCGCCTGTCTCATTCTGCCTC
CTGGGCTGGGCAGGGAGAAAGAACATGGGCCACAACTTGCAGGCAGACATGATGGGCAAAGATG
TCCTAGAAAGTGGCCGCTAGTCTGGCCTGTCTGCTGGGGAGATTAGAGAGGGCCCAGGCCAGCC
AGCACCCATGTTTCATCTATATTTGTATAGATGGAGAAACTGAGGTCCCAGAGGGGGCCAGAGA
GAATCCTTAGAAAGTCCTTTGTTGGATGAATGAAGCCACACCCTAGTTGTGGCAGGCATGAGAC
CCCAGCTAAGGGGCTCCTTCACCTTGTCTCCTCTACCCCTGACCTCCAGATACTGATCTCTCTT
CTGCTGTCCCTGTGTGAGGACCACCCCCACTCCAGGAAGGCCAAGGCAGCTTGAGGGTCAGCCT
GAGCTGGGGTTCTAGCTGCAGCTCTGCCACTGATTCACTGTGTGATCCTGAACAAGTCTATCCC
TCCCTGAGCTCTATTTCCTCAGCTGGAAGATATGGATAATTATTCAGGTGCCTGGAACACAGGA
TCTTTTCTTTTTTTGAGATACAGAGTCTGGCTCTGTCCCCCAAGCTAGAATGCAGTGGCGAGAT
CACAGCTCACTGTAGCCTCAACCTCCCTGGCTCAAGCGATCTTCCCACCTCAGCCTCCCGAGTA
GCTGGGACCACAGATGCACACCCCCATGCCTGGCTAATTTTTGTTTTGATTTTTTTTGTAGAGA
CAGCATCTCACAATGTTGCCCAGGCTGGTCTTGACCTCCTGGACTCAAGCAATCCTCCCACCTT
GGACTCCCAAATGTTGGGATTACAGGCATGAGCCACCGCGCCTCGTCCCAGGATGTTTTCTATT
AGTCCAAGTATATCCCTTCAAATGTAATGAGAGTATCAAGGGTATAGAAACTCTTAGCCAGTGG
GTTAAAGCAGGGAGAAGTGAAGTTTGGGGGCTTTTTAAGCAGCGTCCGCCAAATGGAGATGGTC
AAGGTCAGAGGCGTTACTTTTTAGGAAAGACATGGGGCAGCCAGAGGAGTCAGGGATGGATGTG
CTTTGGGGACCTCTTCAGTGGGTACAAACTGGAGGCTTTGGCTTGGTAACATGGTGTTTTGATG
TTTGGTTACAGTGAACTCCTGCTATTCATTTACTTAGTCTTTCAATAGTGCTGTGAAATCAATC
AAGCAATCGGTCAGTCAGAATAAGGGGCAAGAGAGTGGAGATGAGGCCATTCTTTCAGAGAGGG
GATGGCTGGGGAGGCCCCTCCAAAGAAGTGGCATTGGAACAGACCTGGGTGAGGCAGAGGAAAC
AGTAAAGCAGGTTCAAAGCCTGGAGGTGGAAATATGTTAGGTGTGTTCCAGGACTGGCAAGGAG
GCCAGAATAAGTGGGGTGCAGTGAGCCAGCAGGAGAGTGAAGAAGGTCAAGTTTGGAGGCGGGG
GTGGAGGCACAGACCTTGCAGGGCCTTGTTGTCAGTGCCGAGGAGTTGGACTTTCCCTCTCTAA
GTGCGATGGAAGTCCGACGAGAGTTTTGTGGAGAGCGATGGTTTCCAGCTGTGCATTGTGGGAA
GATGACCCTGGCTGCGTTGAAGGGAGTGGTCACATTCAGGCACAGTGGAGGCAGGGAGGCCAGG
TAGGTGCCAGATGGTGGCGGCTTGGTTGGGATGGTGCGACCTGGCCTGCCCAGGGATAGATTCT
GAAGGTAGAGCCAATGGAATTTGCTCAGAAGGGGATGTGCAGGGGGAGGGAAAGGAAGAATGAA
GGAGCCTGAGCCCTGGGGGAGCCGTGGTGCCGTTACTGAGATGGGTAGGCTGGAGGTGAGGAGG
ACTTGAGGACAAATTGAGAACTGGTGTTTGGAGGTGTTGGGTTTGAGAGGCCACCAGACATCCA
AGAAGAGTCATTGAGCAGGCATTTGGACACACGAGCTGGACACCTGGGAGAGGCTGCCACATCC
TCACTCCTGCTAAATGGCAGAGGCGACTGTGGTCAAGGGGTGGGGCAGGATCCAAAGTGGGCTT
TTCTTGTTTTTGTTTTTGTTTTTGTTTTTGTTTTTGAGACGGAGTCTCACTCTGTCGCCCAGGC
TGGAGTGCAGTGGCGCAATCTTGGCTCACTGCAAGCTCTGCCTCCTGGGTTCATGCCATTGTCC
TGCCTCAGCCTCCCGAGTAGCTGGGACTACAGGCATCCGCCAGCAAGCCCAGCTGATTTCTTGT
ATTTTTAATAGAGACAGGGTTTCACCGTGTTAGTCAGGATGGTCTCGATCTCCTGACCTTGTGA
TCTGCCCACCTCGGCCTCCCAAAGTACTGGGATTACAGGCGTGAGCCACTGCGCCCGGCCTTTT
TTTTTTTTTTTTTTTTTTTTGAGATGATGTCTTGCTCTTGTTGCCCAGACTGGAGTGCAATGGT
GCCATCTTGGCTCACTGCAACCTCCGCCTCCCAAGTTCAAGTGATTCTCCTCCCTCAGCCTCCT
GAGTAGCTGGGATTACAGGCGCCTACCAACACGCCTGGCTAACTTTTGTATTTTTAGTAGAGGC
GGGGTTTCACGATGTTGGCCAGGCTGGTCTGTAACTCCTGACCTCAGGCTATCCACCCACCTCG
GCCTCCCAAAGTGCTGGGATTACAGGCATGAGCCACCGCACCCAGCCTGCATTTTAAATTTTAT
ATTAGTTTGTTATTACCGAAACAACCCATGTTTGTTCCAAACAATATATTAGGAAATACTGGCT
GGGCGAGTGGCTTACATTGCCAGCTCTGGGAGGCAGAGGCGGGAGGATCACTTGAGCCCAGGAG
TTTGAGACCAGCCTGGGCAACATAGTTCAACCCCATCTCTACAAAAAATTTAAAAATTAGCCGG
GTGTGGTGTCATGTGCCTGTGGTCTCAGCTACTCAGGAGGCTGAGGTAGGAGGATGGCTTGAGC
TCAGGAGGTCAAGGCTACAGTGAGTCGTGATCATGCTACTGCACTCCAGCCTGGTTGACAGAGT
GAGACCCTGTCTCAAAAAAAAACAAAACAAAACAAAAAATAGAAAATACAGATAAGCAGAAAGC
AAAACAAAAGCCAAATCACTTAATTTTACCACCCATAGATAAGCCTTGCTGACTCTGGGTACGT
GTGTAAAAACCATCTCTGTTTCAAACATTGGGATCACATTGCCGTTAAGGTTTTATAATCTGCT
TTCCTTTTTTTTTAAATTGTGAAGTGGAAATTTTATAGATGTGTAATTACTGACATGGCAAGAA
AAGTGTTAATGATGGCCAGGCTTGGTGGCTCACGCCTGTAATCCCAGCACTTTGGGAGGCCGAG
GCAGGTGGATCACGAGGTCAGGAGATCAAGACCATCCTGGCCAACATGGTGCAACCCCGTCTCT
ACTAAAAATACAAAAATTAGCTGGGCACGGTGGCATGTGCCTGTAGCCCCAGCTACTCGGGAGG
CTGAGGCAGCAGAATCGCTTGAACCCGGGAGGCGGAGGTTGCAGTGAGCCGAGATCACACCACT
GCACTTCAGCCTGGTAACAGGATGAGACTCCATCTCAAAAAAAAAAAAAAGCAAAAGGAAAAAG
AAAAGTGTTAATGAAACATTCAAGAAGAAGTCCCCAGAGTGGCCCACTGCTGCCTGAGCTTCTG
AGGTCAGCTGAGAAACGGGACTGGAGGGTGTCTGTCAAGGCCGGGGTAAAGTCTTAAAAAGTCC
TGGCTGTGGCCATTCCCCGAGGAGAGGGCTGCATGAGATAACCTCATGAAGGACCCTTCTGGTC
CTAAACCCTGCTTCAGCTGCAGTCCCGATGCAGGTACATGTGGCCGCTGCCTTTTTTGTGATTA
ATTATTGAACATGAGGAGCTGGTGCTCTTCTCTTCTGTTCTCCCGTACCCCGTGTTTTTGCTGA
CTCAGCTCTGGTCTGGAACTGGCCGAGGAATAAGCTCTTCTCGAACTCATTCACTCATAACACT
TTACCGACATCTGCCACCTACCTTCTCAAACCAGGGGTATTTGCTGGGATACACTTGCCCCCCA
AAAAGGTCAGGAACCCCTGTCCAGGAGGTGTAAGTGAGCCAGGGCAGTGCCACGCCCTTTGCAT
GTCTTCCTTCCATAGCCCCCACTTCATAGATAACGAGATGGCTCAGAGAAGGCAGGTGACAGTC
TCAAAACCACACACACAAGCAGGACTGGGATCCAAACTCAGGTCTCTCTGACCCCGAGCTGCTG
TCCTTAAGGTCACCACGAAACATGAATATAAATAAAGCCACATAGAAGGTGACTTGGGCCACAC
ATAGGGAGGCAAGCATCGGGAGTTGGCAGTTCTGAAGGGGGGATAGTGGGGTGCGGGAATCAGA
GGCTCCGTAGAGCTTTGTAGCATTTGTGTTCCTGTAGGCGTGGACGATGAGCAGAGTCTCGCAG
GCACAGACATGAGGTGTTCTGCGTAGCATGCACCGTGCCAGCAACGGTGCAGAGGTGGGGAGAC
TGGGGACATCTGGGAAGGCTGGCGGGTCAGTACACAGAGATGTATTTCCAGAGAAGCCTGTTCC
CTCCGGAGTCTCCTCCAGGGTGTGTGCATGTTCAGCACACCCCCATCCTAAAGAGGGGTGCTCC
TGGAGGCCACCTCATTCGCCTCTGCTGTGTCTGCAGCCTGTGACCTCTCACCTCGCTCTCCTTC
ACCCATCCACATACCGCAAGGCCACCAGTTTCTCTCCTCATGCCCTTGGTCCTCTTCCCATCCA
CTGGGAGGTTGGGAACCCTGGCTCTAATCTCAGCTCTGTCCTGGGCACACTGGGCGACAAAGGG
TGAGTCCTTCTTCCTCTTCGGGCCTGTTTCCTCGTGGTGCACTGGGGGATGAGGGAAGTATTGG
ATGTGCTAGGGTCTAAAGTTCCTTCCATTTCTATGTCTCTGGTTCTGGGCCACTCTGTAGTTTC
ATGCTGTCTGCAGGGAAGTTCCTGGCCTCGATGGCCCAGCTCCACACTGCCCAATTCCCCTCCC
TACATCCTCTCCCTGCTGCATCATTCCTTCTCTACCTGTCCTCCATGATCCTCTTTCCTTTTCT
AGCTGTCTCGTTACTTAATTCCACGAAGGCAGGGGCAGTTGTCCCAATTCTGCAGATGAGGCCA
GGCTCCTGCCACTTTGGAGTGCATAAGAAAGAAGGTCCCTCCTTCATCCGCTGACCTTCCCTGA
GCCCTGGGCTGACCTCTGGGGCACTTCTCCCAAGACATCTTAAAACCAAGCTGGGGCCAAGGCC
ACCACTTGTGGATGGCTAGACCTGGAGGAACCCTGAGTCAGTCTATTTCCACTGCCAGACCTGC
TACTCACAGATGAGTGACTACCTCTCTGAGCTGCAGCCTGCAGCAGGGGGCAATTGAGATAGTT
GTGAGGTCCAGTAGCTTGGGGCTTGAGGGCTGCTGCCTTTGGTGCCCAGCACAGAGCTGTAACT
CCAGGGAATTCCAATTCTGATTTCCACATAATTTTCTGAAAATAATGTTCTCCTCTCTTGGCTC
TGTAACCCCCTGCCAGGGGCACCTTGGCTGTCCTGTCCCTCCAACAATCTAACCCTCTCCCTCA
TGTTGTAATTGCAGATTTTTGCTGGTCTTCAGCTGCCTGGTGCTGTCTGTGCTGTCCACTATCC
AGGAGCACCAGGAACTTGCCAACGAGTGTCTCCTCATCTTGGTAAGTGCTGGGAGTCCGGGACT
GAGGGGGGCTGTGTGAGGTAGCACCTCTGGGCTGGGAGTCCGGGACTGAGGGGGGCTGTGTGAG
GTAGCACCTCTGGGCTGGGAGTCCGGGACTGAAGGGGGCTGTGTGAGGTAGCACCTCTGGGCTG
GGAGTCCGGGACTGAGGGGGGCCGTGTGAGGTGGCACCTCTGGGCTGAGGGCTGAAGGTGGAAA
GAGGCTTCTCATACCATGGCTCAGAGAGCAGCTTGCTGTGCTGTGTTCCCTCAGCTGTGGCTGC
CCCTCTCTGGGCATCATCTCCACTTTATAGCAAGTGTTGGGGGACCTCCTGCCTCTATGCAAGG
GCTTATGATGGGTCCAGAATAGAAGTGAGTAGGCTCCACCCCTTCTCAGGACAAAATCTGCAAA
CTCCACGTGTGGTGGATTTTAGGTCAGTTTTTGCCTAAACACAAAACACCAAATGGAAGCCCCT
CAGAGGGAGACAGCTGACAGCCAGCAACCCCTTTTGTCACCAGGTCAGAGTTAGGGAGTCTGAG
ACAGCTGGGACAGGGAGGGCCTGTTGAGGAGCAGAAAAATCCGATCAGTTCATGGGGACCGGAA
GGGGAGGACACACTAGGGCTTTTAGTCTTCTTGCAGGAGGCGGAGGGGGCCACCTGCCCTCCGG
AATCGTCAAGTCCAGGAAACTCCAGGAGAGGGGTCCGAGGAGGCCCTGGTCCCCCTAACCCAGG
GACTCTTGGCTGGGTCCGCGCTGTGACCTGGGCCCCAGTTCTGGAGACTGAGGACCAGAACTCA
GGCCCCTGGTCCCACAGGAATTCGTGATGATCGTGGTTTTCGGCTTGGAGTACATCGTCCGGGT
CTGGTCCGCCGGATGCTGCTGCCGCTACCGAGGATGGCAGGGTCGCTTCCGCTTTGCCAGAAAG
CCCTTCTGTGTCATCGGTAATGAGGCGCGCCCCGCCCGACCTGACCCCTGACCCCAGGAGCCAG
GGTGACGCCTTTACTCCCAATCCCGACTCTGACCCTGGAGACCCGCACAGATTCAGCGGACCCT
CCCCCTCCCTCCCCAGTCCCCTGCCACATCCCCAGAAGTCCCCGCCTCCGGGTCCGTGCGCGGG
GTAGGCTGGCTGTGATCTCGCCGCCCCCGCCCCTGCAGACTTCATCGTGTTCGTGGCCTCGGTG
GCCGTCATCGCCGCGGGTACCCAGGGCAACATCTTCGCCACGTCCGCGCTGCGCAGCATGCGCT
TCCTGCAGATCCTGCGCATGGTGCGCATGGACCGCCGCGGCGGCACCTGGAAGCTGCTGGGCTC
AGTGGTCTACGCGCATAGCAAGGTGAGGCCTGCAAGCCGCGCGCGGAGACCCGAGGGCGTGTCT
GGGGCACGACCAGAGCGGGCAGGGCGGAGAGGGCGAGGTCAGAGGGGCTGTCTCCGTGCTGGGA
AGGGGTGTGGCCTGCGGGGGTTGGAGCCCTAGCAGGAGGCGAGGTCTAAGCGGGTGGGGCGAAG
TGGATTCTGAATTAGGTAGGGGCGGGCCTAGGAGTCCGGACCGGATCAGGGGGCGGGGCCGGAG
AGGCGGGGCTTGAGGGTTTCCCCTGGATGCTTCTGGGTTTGAAGGGACCACTCGTACCACAAAG
TGGGCAGCTGAGGTGGGACTTGAGGGTGGGGCTGGAGCGGGGCTAGGGTGGGCCCCAGGTGGAA
GCCTGAAAGTGGGACTTGGGGGACGGGGCAAGGTAGGCCGGGTGCTGGAGTCCTGAGCCTGGAA
CCTGGAGACTCAAGGCAGGCGGGGTGAGGGTGGGGGTGGGAATGGGGGTCGGGGTAGGGGGAGT
GCTGGCGGTTTCGCGAAGGCCGGGGCAGGGTCGCCGTGATGGGAGGAGCTGAGAAAGAAAAGCG
AGCCTGGGACTCCGGGAGATGGGGGACCTTTATCCCTTTCCCGTGTGGAAGCCCCCCACATCTC
CCAGGCAGGCACAGCGCTCCTCACCGCGCCCCTCCGCCTGCCCCGCAGGAGCTGATCACCGCCT
GGTACATCGGGTTCCTGGTGCTCATCTTCGCCTCCTTCCTGGTCTACCTGGCTGAGAAGGACGC
CAACTCCGACTTCTCCTCCTACGCCGACTCGCTCTGGTGGGGGACGGTGCGTGAGGGTCTTTGT
AGGGCTGCCCTTCTCCCTGGGATCCTCCCTGGGAACTTCCCGAGGTCTGCCCATCGTGACTCCT
GACTCAGGGTTGAGCGGGCCTGCCCCTGCCCTCTCACTAGAGCTGGGACCCCCCTGAGACCAGC
CCCAATGCTAACCCCCCAACCTGCTCTGGTGGACCTGCCTCTGTCCCCAGCTAGCCCCGCACAT
CAAGACCTTGTGACTATACCCCTTTCCCCTGACCAGCCCAGGAGAGGGAGAATCCATCTATGAC
CCTAACCAGCCCCCGTGGGTGACCAGGGGCCCCTTCCCTCATGATCAGGCTCCTACCTGCCTGT
ACCCCCAACAAGCCGTAGGTGGCCCCCGTGACCAGTCCTGCCTGTAACCTGTTTGTGTCTCCAG
ATTACATTGACAACCATCGGCTATGGTGACAAGACACCGCACACATGGCTGGGCAGGGTCCTGG
CTGCTGGCTTCGCCTTACTGGGCATCTCTTTCTTTGCCCTGCCTGCCGTGAGTTGCCTCCTGCT
CAGTTGGTGGGGGAGGCTGAGGGTGGGACCTGCTCACCCCTGTCACACATTTGCCTGGGGAGCC
TGGGGTACCTCAGAGGGGCAAGGATGGGGACACCCTTGCAGCCTCTTACTGCCCCACCACTGCC
AGCACATTCCCCCAACCATGCCCTATCCCTCTAGGGCATCCTAGGCTCCGGCTTTGCCCTGAAG
GTCCAGGAGCAGCACCGGCAGAAGCACTTCGAGAAGCGGAGGATGCCGGCAGCCAACCTCATCC
AGGTACAAGATGCCCGGGAAGAAGCCCTAGGAGCAGGGCCGTGTGACTGCACTGGTGTGTCAAC
CCTGTGTGCTGACCCATGTCCCCAGCCCAACTGTGACACCACTTTGAAGCTCAAAAGGGCTATG
TGACTTTCCTGGAGCCACATGCCAAGTCACACACAGAGCCAGGATGCTCACTGACAGTGCCAGA
AACTTCCCCCTGCACCAAATGCCTGGTTTAGGGCTAGCCAGAACTGCCCTATTGCCCAACTCCA
GCCCAACTTCACAGGGCCTGGGGAGGGGCGGAGCCCAGACAGGAGCTGGGAGGCCTCTGGTGGA
GACCCACTGCTGCCCCCATCAGCTGTGCAGCAGTAAGCCTGCCCCACTTCCAGCACCTTCATTC
ATTCATTCATTCCACAAGCACTCACACAAATGAAGAGAGGATGGTGCAGACAGGGCTTTTAGGA
ATCTCAACATTTAGCAGGTGGGTGGGCCTGCAAAGTTGAGCTGGGCAAGGAGACAGAAGGAACA
GCCAGAGGGGTGGGTAGGAAGCCAGGAGTGTGTGGTCTCAGTTTCCCTCTGTGCCATGGCGTGT
TGTGACTGTGTGTGGGCTGAGGCCAGGGCTCTGATTTTCTGTGACCCTGGGACTAGGCCTTGGC
TTTTTCTAGTCAGGGGTGTGTGTAAGGGAACTCCTTGCAGGAAATGCTTCAGGGAATACTGGCC
CAGTCCACCTCTGCCTGCTCCCTAGCCTGTCACCCTCTGAGCCCCAAACCCAAGCCTCACTCAG
CCCTTCCTTATCTCTCCTCCCCAGGGTAGAACCCTCCTCCCTGTCGCTGTTATCTCCAGCCACC
AGCTCAGCCTTTTCTTCTTAGCGTCCCTTGGGGCACAATGGCCACTGTCCCCTGGGAGCTGGCC
TTCTGAGGGGCATTGTAGGGGCAGGCGGCTGGCACTGTCCATGCCAAGAGACAGCTAGTATTGT
GGTGCCCATAATTAATGAGACAGCTCTGGCAGCTGCTCCCCAGCCCATGCCAGGGGCCTAGGCA
AGCAGGAAGTAAAAATATTCCTGTCTTAGGAGGGTGCCTGGCTGGTGAGGTCAGCAGTGGCAGC
CCCTTAAGGACAAGAGGCATCCAAAGAAGGGAGGCGTCCAGGGCCCAGCTCTGCCTTGTCCTGC
CTAGCAGCCTCCATGCCTCCCTGCTCTCACTGATGGCCACACTCACAGTCCCTCCCTGGCTCTT
CCTACGTGGAGGGAGAGGGACAGTCTCTCCTCCTGTCTGACTTTCATCCCTCCTGCTGCAGAGG
AAGCTCGGCAAGCCCATGGATGCTGAGGGGTTGGTAGGTAATGCTTTTCCTCCCCTCTCAGTCC
TTAGTATTCCCATGGGCAGTGACAGGCAGCTGGGAAAGATGGAGTGGCATTGCTAGTAATAATA
ATGACATTCTGCAAACATTTGCAAAGCACCCACATGTGCCAGACACTATATTTGCACTGAGAAT
GCAGCGGTAACAAAGACACAGCTTGCAGTCTAGGGGGAGAGAAAGTTATCAGTCAAATACACAA
TGAATAGGTATTCATCAAATGTGATAAGGGGAATAACAGGAGTGTTTAACAGAGGAACCTGGCA
TGAAGTGCCTACTATATACCAGGCACTGAGCTTGGCATTTAATATACAACTGTTCATTTAATCC
TCACAACACCCTGGTAGGCAAGTAGTGTTACATGCGTTTTACAGGTGAAGAAACTGAGGTCCTG
AAGCTTTACTTAATTTGTCCTCACTAATAAGGGGCAGAGCTGGGATTGGAAACCCATGTCTGCC
TGACAACAAAACTCCAGCTCTTAACCTCTATGCTCTATGGCCCCTGGCTCTGACATTGATCTGC
TTTGTCCCCTCGGGGAAGTCGTTTCTCCCTAGGCCTTACTTTCTTGGCTGCCCCTGTCAGTGGC
TGTGGAATTGGAACTGGCCTCTGGCTCTGGGTAACCCACAACTGGACCAAGGACTGGGTTCTTT
CAGGGGAGAGGGCTGGTGGGGACTGAGAGGCCTCACTGATGCCCCATCCCCCACGTCCCCCATA
CCACCAGGCTGCCTGGCGCCTGTACTCCACCGATATGAGCCGGGCCTACCTGACAGCCACCTGG
TACTACTATGACAGTATCCTCCCATCCTTCAGGTAGGTCCTGCTGGGGGTGGGGGTGGGTGGGG
GGCTGGCAGCAATGCCCTTTGAGGACAAGTGGCTGAGACTCAACCCTGGAGGGTGGAAAGGGGG
TGATGGCTCCCAGGGGAGCACCCTGGGCTGGCTCTGCAGATCCTATGTAATTCAGAGCCTGGAG
TGTCCTTTATTCCACTCTGCTATGCCCAGGGAAGAGGTGGATACTGGACATAGGGCATTGGTGA
AGCCCTGCCCCATGGCTGAACCCCGGCCCTGGAGTGCTGGCAGTGTGCATGCTCACCCATAGGT
GATGCCATCTAGGGCAGTGATACAGCCAGCTCGTCTCCCCTCAGTCCTGAATGGGGGACAGAGT
TAGTGGATATTGCAGCTGGTTGGAGAAAGTGTGGGATGGGCAGAAACATGTGTTCCAGACTTAG
GTGCATGCTCCTACCCTGAACCCCCATGTAGGAATATGTGCCCACTTTTACTGGGCCAGTCAGC
AGCCTCAGGGCCCAGAAGTGGGTCTGCCCCACTGGCCCGGGCCAGGCTAAGACTGCATGGGGGT
GGCACCAGGAGAGGGATTATTAATAGCATCACAAAGGTGCGTGTGGCCAGGTGCCTGGCTAGGC
CCTCAGGCAGGAATGAAAACCATGGAGCCCAGCTCGGTCCCACGGGCAGGCGGGCATCAAGCTT
GTGGCTGGGGCAGGATGCTGTGCTCAGCGCTTCTGCGGGTGCCTTTTTTGCCTCATTAGTTCTC
CAGGAGAAGCTGTTGCGCGATTCCACTCGCCTCTGCACCTGGCTTCCCCATCCCTGCGCCCTCC
CAGCCCTAGGGGCTTCCCAGGGGAGAAGCAGGAGAGAAGGGGCCGGGAAGAGGGGCTCCCAGCA
TCACACTGGTGCCCCCACATCTCTCCACCCGGCAGAGGCAAGCTCCTACCTCAGAGTGGGAAGG
TGACTAGATTAGAAGAGAGGCCTTTTGTTCAAGGCTGGGCATTCGAGAGTGGGGCAGGGCTGAT
TTGTAAGAAATACAATGTGTATGAGGGAAGATCTGGGGGTGGGGGTGGTCAGATGGTCTAGGAC
CTCTGCCCAGAGGAACAGAGCCTAGTCTGGAGATTCTGGATCCACTGGGCATCTGCTCCCTGGA
CCTTAGTTTGGTCATCCATGCAATGGGAAGGCAGGCAGGAGAGAGGGAAGATACCCTCCTTGGC
TCATGGCAGCAGAAAATGGCCTGGAGAGTTAGGAAGAAGCCACGGTCCCGAATCTAGTCTTCAA
AAACCCAGTGGCAAGAGTGAGTGCTGGAGGGGAAGGAAGCTGGGCTAGCCACCCTGTCACACCG
CCCTCCTCACCTCCATCTCCCCTTAAAATCCAGAGTCAGATCTGGAGGGAAAGGATGTCAGGGC
CACTGCTGGCCTCTCTGAGGAGGCACGGCACGTTCAGGCAGTCAGCTTGATGGGCTGAGAGTAA
GCTCCTTGCACGGCTGCCCTTGAGCTGTGCCAGCCTTCACATCCATGCTTGGCATCCCTGGGAA
CTGGGGGTAGAAAGGGAGTGGGGAAGGCCAGTCTCCCACTGTCCACCCTGTCCTATTCTGGCCG
GGCTGTCAGTGAACACCTCTAGAGCAGCAGGACCTGGCAAGATCTGAGCTCAGGAGCTCTGTGC
CCACTGGGTGAGGGGGGTGGTGCCATGGCCCTGCCTGCCACTGATGGTGCCCTCTCCTGCAGAG
AGCTGGCCCTCTTGTTTGAGCACGTGCAACGGGCCCGCAATGGGGGCCTACGGCCCCTGGAGGT
GCGGCGGGCGCCGGTACCCGACGGAGCACCCTCCCGTTACCCGCCCGTTGCCACCTGCCACCGG
CCGGGCAGCACCTCCTTCTGCCCTGGGGAAAGGTAGGGGCCCCGTGGGGCTGCCACCTCCTCTT
GCTTCTCCTCCTCTTCTTCCATTCTCTGTCTTCATCCTCTCTCAGACCTGCCTTCAGCCACTTC
GTGGGTGTCTGGGCCTGTTTGGGGGACTCCTAGGCCGTCAGAGGGCCAGACAGGTACAGACTGA
GGGCCTCTCTGGAGGGGTCAGGCTGGACTCACCTTTTTGTCCTGTGGGGCTCTGAGGGTCTGAG
TGGGAGAGATGTCCATGCCATCCTTTCCCATCATCTCCTCCGTTCATGGCATTGTCCTCTTTCT
TTCTCTGTTCCCACGTCCCCCTTACTCCTCCATCCCTGCACCCCCATCCCTCCCGGGAAGCTGG
ACAGAGGAGGAGGCCACTGGCCACAGGTGCTTACGGCTCAGGTAATCGTGGGCACCATCCTGGG
GTGTGAGGGCAGGGAACCCCCTCCCCCGGCATGTTGCTCTAGGAGGGACTTAGTACTGAACTCG
CCTCTTGGGAACGAGATGCCAGCTGGAAAGAGAGCTCTGTGATTAAGACCTCTGTAGGACCTGC
GTAAACTCAGCCAGTGACTAATGTTCCTTTCTTGTGCAGCCCTTGTCTGCAACTTTGCTAATCC
CAGAGGGTCCTCTTGGGGTTCTGTTTCCTTCCTTACTGAGGTACACTTTGGCGCTCCCAGCCAT
CTCTTTCCCCTTGAACTGTCTCTCCTGAAGCTTTTTACAAAATCTTGAAAGAAGAACAAAGAAC
AAACAATGCTTACCCCCACATAAACGTTCCTAAAGAGAAGAATTTCAGGCATGTTTGGATAAGG
GAAGCCCGTCCACTCACAGCATGGGAAAGTGGCTGCTCTTGTAGAGGTTTAGCTTTGAGGTCAG
GGGTTAAATCCCCCTCATTCTTTTTCCCTCGTCCTTTACCCATCTCTTTGTTTTGTCTCATCTT
CACACATCACCCCTGAACCAGCCAAATGGACATCTGGCCTTTGAGCTTCAATGTCATTCTGAGC
CCCAGGCCCAGAACAAAGAATAGGAGAAGAGGCGAGCCGCCCAGTGATCAGCAGGGGAGGCGTG
GTGGAAGCTCGCCTCTGCCACCCGATGCTGTGTGTCCTGGAGGAAGCCTTCAGTCATTCTGAAC
CTGTGTGTCCTGGCGGAAAATCGGCAGTGTTTAGGCTGATTGGGGCTCTGGGTACTCTAGAGTT
TCTGGGCTGAAAAGGCCTAGGGAGGAGTTTTGGTGCAGAGGATGCAGGGAGCAGCCTCCCTTCC
TTGTTCTCAGTGAGGGCACTTCTGGGCCAGGGGCTGAACAGGAAGTGGGTGGAGAGGGAGAGTC
CCTCCAGGTCTGGGGCTGGAGGGAGGGGAAGCGTGACTGCCAGGCCAGGCTGAGAGCTCCCTGG
GAGAGTAGGGCCCCTCTCAGATTCTCTGCCCGAAGGTCCCTCATTGTTACCTTCATCCCTACCA
AACATAGTTTCCAGGCGGAAACTCAAGAGTTTTTGTATCCCAGCAATCCTGTCCAGGACCTGAG
CCAGGGCTCTCTGTGCTTCCAGTTCACTCTGGTCTCATTTTGACTGAATAGGAAGGCAAGGGGA
ATGGACATTAGCCTGGGGCCTAGTACCTGTACACTCCCCACGGCTCCTTTGGTCCCTGACACCC
CCAGGGCCCAGGAACAGATTACTACTAAGGGCTTGGCTGTCACTGACTCCCAGCCTCGTATCTA
TCTGCCTGGGAGGGGACTTGGCGAGTGTCTGGGACACAGGGTGATGGTAGTAAAGTGGCCCTTT
CTCCCAGGGACCCCAGGGGGCTGAGCCATGGTTTGTAGATCAGTCACCAGGCTCAGGCCCAATG
GCCGTATCGTTAATCGTGGCTGTCTAGACCGCCAGCCAGGACACCCTTCCTCCACCAAAAACAC
ACACACATATACACACTCGGTTGGCCACACCTCTGGTGGTGCTTGGGGATGGACCTGGGCCAAG
TGTGCCCACAGGAAGTGGTGCTAAAGGAGCAGGATTTCCTGGGGGATGGCATTGCCAGGGCCAG
CACCTCTCCTCTTCAGGGTTTTGCAATGAGTTTATCAAACCATGGTCTCCAGACCTCCTGCATC
AGCATCCCATGGAGTGTTTGTTCAAAATGCAGATACCTGGACCTGTCTCCAAGCCCACAGGATC
AGAATCTCTGGAGAAGAGACACAGCCTTCTGCATATGTAACATGGTCCCCAGGGGATCCCTATG
CAAAGTGTGAGAACATCTACGTTCTGGTGATGGGGCTGGAGGATGATTTAGGTGGGGGTCCTTC
TTTCCCACCAGCTACCGGAGGCCGGCCCCATCCCAGCAGCTCCCCACACTCTGCCCACAGCCCC
TTTCCCGCTAACCTCTGTTCGTGTCTTATTCTCAAACTTCCAGCCAGATGTTTAATAATAAACG
GAGTTTCTTTCGGATCCACGCCAGCTGGAGACCGAGGTACCCCCTCGCCTGCTCCTCCTGTCCC
CACTCCTCCTTGCCCCACCCAATTTGGGGGCTGGAGAGGGGACAGGATGGGCCAAGGACAAGGT
GCATGTGCCTGCCGCCTGCCGTCTGCCTTGGGTGCCCAGTGAACCCCTCGCCTTTCTGGGATGC
CTCTGCTAGGTCTGGGGCATGGGCCCAGGGTGCCAGGAGGGGCACGTGCAGGAGCCTTAAGGAT
GTGTGCTGGCTCTGCTGCTGATGCCCTCTGTGATCTTGGCCAAATTGCTTCCCTTCTTGGACCT
CAGTTTTCCGTCTGTGGAATGGGCAGATTGAACTAGAGGACCTCTGAGGGTCCTTCTCACTCTG
ACTTTTGCCAGGCCTTGGGCTTAGCTTTCTGTCTCCCTGCTACCCACCTGGGGACCTAGCAGAT
GCCTGAAGGAGCTGGGTTTGGCACCTTTGCCAGCTCCAGGACCCCAGCTCTTTTAGCGGCATCT
CCATGGCATTCCTTGGTACCTGCCCTGTGCCAGGACTTGCTCTGTGCACTAGGGACACGGTGAT
GAATAAAACATCGTCCTCGGAGAGTTGCAAAGAGTAATTGCAGCTCAGTGTGATGAGTGCAACA
AAGGGAGGAAGCACAGGGGCTGTGGAGCCCGGGTCAGGCACACAACCCAGAGGAAGCCCTAAAA
GCAGTGGGTTTCAGGGAGGGCATTCCAGGCAGTGAAGCAGCACGGACCCAAGCAAGGAGGCAAG
AGGGAGCACAGTGTGGGGAAAAAAGCTGGCAGCCCAGTTTGGCCCCCCTGAGACTGGACATCAG
AGCGAAGGTTGGGGGCAGAGCTGGGTCATGCAGAACCTAAAAGCCCAGTGTGAGCTTCACCCTG
TTGGCATGCTTGGCAGCCTGAGCTCATGGAGTCCTGGGTTTCCAAAGGTGCCTCTGGGGCCCTG
TGGGCTGGGGTCCCGGAGCTAGGAGATGAGTGGCAGGGCTCCTGGCTGCCCATGTGTGCTTCTG
CTGAGAGGTGTTTTTTCCTAGAAACGTGGGTTCTGGACCACAATCTAGCTACTACCCACTATTC
GTGTGTCCTTGGGCAAATCACCTGACCCCTTTGCACCTCAGTTTCCTCAGATGTAAAATGCAGA
TGTAAATAGGGTCTGCTTCCACTAAAACTGGACCTGGCTCCAATAAATATTGGCCTTATTTGAT
TTGGTATGTTCAGCAGTTAAGATCTTATTTACCTTAAAAGAAAATAGCTTAGAAACCATGGCTG
TGGGCAATGGTTTGAGCTGAAGCGTTGAGCTAAGGAGTCTCTGGGTCAGGCCCGCCATTTTGCA
AGCCTCCTTTGGCTGTGGAGGGTGTGGACAGGAAGGACACAGCCATGGCAGAGGCCAGTGGCGA
GGCTGTTTCCATAGTCCCTGAAGAGGGGCTGTATGTGGCTTCAGAAGTCGTAGGCTGCCTCTGT
TCCCCCAGTGAGAGTGGCCCTGGGAGGGCAGATCATGAGGCCCTAAAGAAAGTCCTTGTGGAAG
TGAGTTCAAGGGGTTGGGACCAGCAGGGCAGCCTGGGCAGAGCTCGGCCTCCCCAGCAGGGCAG
CCACAGTCCAGCCCACCCCATCTTTCTCCCAGGTACCAACAGATCCTTAGCCAGAGTTTCTTGA
CCTTGGCATCATTGACATTAGATAATTCTTTGTGGATTGCGACTATCACATGCATGGTAGGATG
TGGAACAGCACCCTTGGCCTCTACCTACTAGATGTCTACCTACTAGACATTGCCACATGTCCCC
TGAGTGGGGGAACTTGCTCCAGTTGAGAACCACTGCTCTACACCCTATCCTGATTGCTTGACCC
AGAAAAAGAACATAGACTCTGAAGTCAGAGAAGGCTGGGTTCCAGTTCTTGCTCCATCTCATAG
GAGCTCTGTGACCTTGGCCAAATCATCTCACCTCTTTCCGTGTATATAAAATAGGGATGGTAAA
GACCGCCCACCACACACAGTGGCTGAGAGAACTGAAAGAACTAAGGCAGCCTGATGGGACCCTG
CACGTTCTGGGAGCCTGGGAGGGGAAAGAGCTTTTTGGTGCCCACTGTGTGCCAGGTTTTCTTC
CACCTTCTTTTAGCTTTCAGACTTAGAGGAGCTGATATGAGCCATTTTCTGGGCCCTGCCCAAG
GAACTGGAATCCAGACCCACTCCTGGGAAAACAGTGCCTTCTTGCATTAAAAAGGAGCTCAGGG
CCTCTGGCTGGCTGGAAACTTACAGAACCCCACAAACCAGACTGGAAGCTTCCTCAACCATCAC
AGAATCAGAGCTGGAAAGGGACCTTTCAAGGTCTCTAGTGCAAGCCCTCGAACTCCTTCCTCCA
TAAGTCCTGCTGAGTGCAAGCTCAGCCTTGCTTGCATTACTTCTTTTGACAGGGAGGTTGCCCT
CTTCTCCAGAACGCCATTGGAGCCTTATGTGGTGGGCGCAGTGATGTGGAGTTCCAGGCCCTTG
GCCAAAGTGTCAGGGCCTTCTCCACTCTCCTCTCTTCCTCTCCCTCTTGGAGGAGTGGAGGACA
GGAGAAGGGGTTGCACGGACCGGGCAGGGTGGAGGGTGCTTGCCAGCATGAGGGAGCCTGCAGG
AATGGCTCTGCAGAGGAAAGTTTGGGAGAGCCAACCTCAAACTTTGATTGGAATCTTTGGGATG
TGTCGATGGAGTCCTGGGCCAGTGGAGAGCATGCTGGATAGTGGGAACTGGGAACTGTCCCTGT
CCTTTTGATACCCTCAGGGCCCCCATCCTTGACCTATTAACCTCTCCCCTCTGGGCAGAGGCCG
CCACCCCTAAGGATGGCCCTACATCCTGGCCACCTTCCTGAAGGTGCCCCTGGTTTCGAGGTCT
CCCTCACTTCAGCACCTTGGCTCCCTCCCCTCCACCCCATGCCCTGGGCATGGGAGACCCCAGT
TCCCACCCAATACCGAAACCAGAATCTCTTCTCTGCTCAACCCTTGATCTGAAAGCCTCTGAGA
GTAGGTGACACGGTGTCCTCCCCCAACTCCTGCCCCACTTCAGGTCTCCTCTAAGGCTGGCTGG
TACTCTCTTGATCTGGGGCCCCAGACATTTGGGGTGGGTCCCCCTGAACCCGAACCTGAACCTG
TCGGAATCTCTAGTGGCAAACCACAGCAGGGGAGGCAGCGATGACGTCACAGGGCATGGGATGG
AGGACATTTGCATAGGTCCTACCCCAACAGCCATAACATTGTCCTTGAACCTCTTGGACACAGA
GTTACTCTACCAGATCATGGTTAACCAGGGCTTGTCCTGGGGAATGAGGCCTCTGCTAACTTGT
TTTCCTCCAACGTTTGGGGAACGTGGTGGGGAGTTGGATGCGGGAAGGGAAGTCTGAGTGCATG
TGTAGACGTGCACGTACACGTTCACATGCACACAGACTGTGTAAATGCTGTTGAACCGGGAGCT
CAGTAGGCAGTATCGGGGGGTAGATCTTTAGCGGGGATCCAAGACCACACAGGCAGGATGTCAA
TCAAGGTGGGCTGAACTTAGGTAGGAGAGACCACAGAAGAACATGTTATCAAATAGAGACCCTC
ACTGAGAGGGCAGGCACCAGGCATGGCCTGGGCATACATGTGGGCCCAGCAGCTAGAACCATGG
GGCCGAGGCCCTCAGTAGGCCATCACGCTGCACACCTCCAGGGCACCACTGCATGGAAGTCTGT
GTGATGGAGTCCTTATTTTGCAGTGCACTGCCTGCACAGCTGTACGAGGCCCTAAAACCCTGAC
CAGTGTGGGATCAGATAGCCGCAGCAGTGGGGAGTGGAGGAAAATATGAAATGACACATGTAAA
CACACGCCAACATATGTAGACACACCAGTGCCTGTACACCCACACTCGCACATGTGTGTGCACA
AGGTGTGTGTGTGCATGCGCGCACACACACACACCCTCGACTCCACACAGCCTGCCCCTGTGTA
CAGTGAGGACTGGAGGCCTACCTCCGTGTGCATGGCCAGGTGAGCTTCCGACCCTCAGCAGCCT
GTCTGTGCCCAAAGGGGTCGCCACCCCCACCCTCTGCCCACTCACCTGCGCCAGGCCCAGCTCC
CACATCCTCCTGTCTGTGTCTCTGACTTTGGATTTCTCCCACTCCTTCCACTCTGACATCTGTC
TCCTGTGCACCCCTTATCTGTCCGGGTGATGAGGGGTGGGATGTGGGTGGTGGGGAGAGGATCT
GCTTCGACTGACCCCGAATCAGCCTCTTGTAGGAGTGAGGGCCTCCCTTACCACATCCTTGTTC
CATCCCAAGAAGTCTCCGCTTGGCGGGGAATCCAGACCTAATAGCCACCCCCAAGTCCTAAGTC
AGCTTTGTCCCCACTCACCCCCACCCCCAGCTCTGGCTAACTTGGCTCTCTCCCAACCTGCCTG
CCCTGCCAGCAGCCGGATGGGCATCAAAGACCGCATCCGCATGGGCAGCTCCCAGCGGCGGACG
GGTCCTTCCAAGCAGCATCTGGCACCTCCAACAATGCCCACCTCCCCAAGCAGCGAGCAGGTGG
GTGAGGCCACCAGCCCCACCAAGGTGCAAAAGAGCTGGAGCTTCAATGACCGCACCCGCTTCCG
GGCATCTCTGAGACTCAAACCCCGCACCTCTGCTGAGGGTAAGCCCCCGGGGGGCTGAGTCCGA
TCGAGGGCCGGCTGAGGGTGGCAGGGCGGGGACAGTCTGGGCTGGGAGCAGAAAGATCTGGCTC
GCGTCTCAGCTCTGGAACTGATGGACCGTCTTTGGGCTCTGGTCTTCTCATCTGTAAAATGGGC
ATTATAATATTTGCTCTGCTATACAATGGACATCAGACTGTTTGGGAATCACGTGGAGGGTGGC
ATTTCATAGGGGTTAGCAGCACAGGCTTTGGAGCGAGACTGCCTGGATTTGCATCCTGGCTTTG
TCACTTATCTTAGGCGAGTGACCTAATTTCTCTGGGCCTCATTCTCCTCATTCATAAAATGGGA
ACAATAATAGCATCCACTTCCTAGGATTATTGTGAGGATTAAATGAGATAATCCATGGAAGGTG
AACAGAAGACTTTTGGGAATATAGGAGGTGCTCAACAAATAGCTAAACACAAATGTACTTTGGA
AGCGGCAGGAATGCATCCTTGATGAGCCCTGACTATGCACTGCCCAGCACTGGCTTATCTCATT
TACTTCTCCCAGTGACCCTGGAAAGTAGGTTCTGTTTTTGTCCCCACTTTACAGACGTGGGCAC
TGAGATCTCTCAGCTTGTGAGTGGCAGAGGTGCTCTCCTCAAGCAGGTCTATTCAGCTCCAAAG
CCCAGGCCTTCTTCCTACCCCTCTTCACCCAGCCCAGGCTGCAAGTATGAGACTGTGGACCATG
CTGATGTCACTTTTCTGAGTTTTGGCTCTGCTGGAGACTTCCACTGTGATTTAGGCAGGAGACT
GCCCCTTTCCATGCCTCAGTGTCCTCTTCAGGAAAATGAGAGGGTTGGACCAAGCCCCCTGGAA
GACTTCTCCCAGCTTTGACATACTTTAGGCCCCTGGGTGGGGAGAGGTGGGGTGAGTGCACAAG
GATGGGGGATGGGGACAGCTCCCCCTTTGGCCTTGGAGGGAGGGGACCCAGCCATGCTGTTTCT
GCCTGTGCTGTGTCAGCCCTCAAGATGCTGCTGGCTGACACCTCAGAGTTTACAGAACCCCATA
GACCAGTCTTTGAGAGGTGCTGCCCATAGAAGCCTGGGCACAGCTGGTGCCCAGGACCTCCCTT
CCCTGTCCTGGAAGCTGTCCGGGCTCTTGCCACTGCTCCTGTGAGGCAAGGACAGCATCACCTC
TCTGCGCAACACAGGGCACTAGCCTCAGAGAAGCTTCTCTCATTCAGGGAAGAATGGTTTGTTT
TAAGGGCCTTCCCTAGTAGCAGAGCTGGGCCTGGGAGCCAGCCACTTGGGTGTCCTTCCATCTT
AGCTGAAGGAGAAGACTGTGTGCATGATTACATGCAGTGCCATCAAACCCCTTATCTCTGGGGC
TCACTGTTCTGCAAGGTGGAACCAGTGGGGCTTCCTCCAGGGCTTTGTGCAACAAACTTGCCCT
GGACTAGGCCAGCTGTGCCCTAAAGCAGCTCCCACCTACCCACCAGGCAGATGTTTACCCTGAC
TGGCCACCGGGGCAGATGGGCATGCAACACACTGGGGCTTTCCACCCTGGGGAGGGCAGTGAGA
CACAGGAGCCCCAAGAAGGTTCTGAGCCCTTTCTGGGCCTCTGTCTTCCTATTGGACACTCTAC
TGGTGGTTTGGCATACAAGGGTCGGATGGGAGGTTGGGAGTGGCCCCTTTGGTGGGCCACTTGC
CCCATACCTGCCTTTTCAGATGCCCCCTCAGAGGAAGTAGCAGAGGAGAAGAGCTACCAGTGTG
AGCTCACGGTGGACGACATCATGCCTGCTGTGAAGACAGTCATCCGCTCCATCAGGTAAGACTG
AGGCCTGAGGCGAGCACCCCCCTCCGTGTGCCACCCACTGCTGCTCCCCATCTGGGCGGGTGGA
TCACTTGAGGTCAGGAGTTTGAGACCAGCCTGGCCAACATGGTGAAACCCCGTCTCTACTAAAA
ATACAAAAAATTAGCCAGACGTGGTGGTGGATGCCTGTAATCCCAGCTACTTGGGAGGCTGAGG
CAGGAGAATCACTTGAACCTGGGAGGCAGAGGTTGCATTAAGCCAAGATAGTGCCATTGCACTC
CAGCCTGAGCAACAAGAGTGAAACTGTATAAAAAAAAAAAATGTATTTTTTATGGTGGGTCTTG
GTCAAAAAAGTTTGAAAGCCCCTGCTCTACTGGCTGTCTCCATGCCTCAAAAATTAAGCAGGGA
CCAGGGTCTCCTAGGCCACGGCCTAGGCATGGATCTGGCCTCCAAGCTCCTTGGTGTGCCGTCC
TGGCCTAGTCTCCATCTCCAGCCTGCAGCTCTAGCTCCAGAAACCCCCTCAACCTCCAAGTTTT
CTGGGCTCCCCACTTTCTTCCCTGGCTGATACACCTGCCCACAAGCCTCTTTTCTTGTATTCCT
TTTAAAATTAGAGTCAGGCTGGGCGTGGTGGCTCATGCCTGTAATCCCACCACTTTGGGAGGCT
GAGGCAAGAGAATCACTTGAGGTCAGAAGTTCGAGACCAGCCTGGGCAGCATAGTGAAATCTTG
TCTTTGCAAAAAAAAAAAAAAAAAATTAAAAAATTAGCCAGGCATGTTGGTGTGCCCCTGTAGT
TCCAGCTACTTGGGAGGCCAAGGTGGAAGGATGGCTTGAGTGCAGGAGTTTGAGGCTGTAGTGA
GCCGTGATTGAGCTACTGCACTCCAGCCTGGGTGACAGAGTGACACCTTGTCTCTAAATAGATA
AATAGATAAAATCAGAGTCAGCTGGACCTGTCCCCACTACCCCCCACAGTCTGAAGCCTCTGCT
TACCCCCTTTTCACCCATCCATTCTCTCCCCTCCATGTGCCCAGTGTCAAGGACATAGTACCTG
ACCTGCCACCTGACCCAAGCCAGCTCTCCTGGGCCACCCCACCGACCTCTCAGCCAGCATGCCC
CAAACCTGCTCCTCTTCCTGGGCTGGTTCCCATTTCAGGCAGTGGCACCACCATCCACTTGGTC
ACCCAAGCCATGGGAGTCATCCTGGACTCAGACCAGGGAGACTTTCAGCCCCACCTCCTGAACT
GCTCCAAGCTCTTCCATATCTCCTCCCCCATCCCCTAAAGCTAGTTTCCCTGCCCACGCCTCTG
CCCCTCCAGGGTATTCTCCACACAGGCTGGCAGAGCTGGCATGCTAAAGCAGAGTAACTGAGCC
GGGAACAGTGGGTCACACCTGTAGTCCCAGCACTTTGGGAGGCTGAGACGGGCAGATCGCTTGA
GTCCAGGAGTTCGAGACCAGCCTGGGCAGCATAGTGGGACCCCCATCTCTTTAAAAAATAAAAA
TTAAAAAAAAAAAAAACAGAGTTATTGATGTGCCTGCCCCACAATGTCCCATTGCCCAGTCTCT
GCCAGAGATGCAGGGCTCTTCAAAGCACACCCCAGCCTCCTTCTCGAGGCTGATCTCCTGTCTT
CTCATACCATGGCACCCTGAACCCCATCCTCCACCCCGGCTGGTTGATTAACCCCTTTCCATCT
CATCCCTGTTTCTGCTACTTGGAGGTAGGGGAGGCTGGGAGACGCAGCAGAGGCTGTTCATGGT
GGCCAAGCAGGAGGTGCCCTGGTGCTGGACAAGGGGATAGCAAAGAGATGGAGAGGGTGCTGGG
AAAGAATCCCTGCTCTAACAGGACACTCCCTCTGAGCCCCCTCCCCCAACAGGATTCTCAAGTT
CCTGGTGGCCAAAAGGAAATTCAAGGAGACACTGCGACCGTACGACGTGAAGGACGTCATTGAG
CAGTACTCAGCAGGCCACCTGGACATGCTGGGCCGGATCAAGAGCCTGCAAACTCGGTGGGTGC
ACCGGGCCTGTTGGGAGGCAGGGGCAGGGAGGCAGCGACCCCAGCCCTGAATGAAGTCTGAGGC
CAACCCCCGAGCACCCCCAAGGGCTCACTGCTGCAGCTGAGTTTCTAGACTGAGGAGGTCCCTG
GCTTGCAGTGCCAGCCTGAGGCCAGACCCAGCACCCGTCCCAAGGCTGAGAAACAACCCAACCA
TAGTTTCTCTGTGAGGTGGGTCCTCTGTGCAAGCCCTATTGACTCATATGCCTTGGGGGCTTTG
TTTAGAATGACTGTTTCCTGGTCCTAAAATAATCAGCCTAAAATAATCACTGACCTCCCATCTC
CCAAGAGAATAGGCAAATTGTCCTCACTGCCCACCTCTTGCCTCTGGAATCCTCCCACATCTCT
CTGGTAGACCCAGCATTAGGAAGAATCCTCACCCCAAGACCCACATCACCTGCACCTTGCCCTT
CTCCTCTTCTGGCCTGCATTTTCACTGTCCACTCATTCATTTAATCACTCGAGAAATAGTCAGT
GGGCATTTACTACTTGCCAGCCTGTGGGGATATGGCAGAAAGCGCAGCAGAGTTCCCACACTTA
TGGAGCTTTCATTCTAGTGGAAGGAGAATAAGTGATAAATAAGTAAAACTGGAATATGTCAGAC
AACGATAAATACTAAGGAGGAAAATAAGGGTCAGGCTGTTAGGGTTTGCAATTTTAAATAGGTG
AGAAGGTTCTAGAGGGAACAGCAAGTGCCAAGGACTTGAGAGGGGCAAGTGGCTGGAGTGTTGG
CTTCAGCACAGCAGGGAGCCTGGTGTGGCTGCACTGGAGTGAGAGGTAGGAGGTGAGGTCAGAG
GGCTAACGGCACTCAGAGAGTGTAAAGTCCTGTAGGCCCCTGCAAAGACGTTGGTGTTCATCCA
GGATGAGCTGCAAGCATTGGAGGGTTTTGTGCAAACGACTGACAAGATCTGAATTGTCTTTTAA
AAGGATCACCCTGGGTGCTGTGTTGAGGATGGACTTAGCGGAGTGAGGGTGGACACAGGTAGAC
CAATGAGAACCTGCTTCAGACTTCCACGCATGAGACCGTGGGGACTTGGAACAGAGTGGCAGCT
GCAGAGGTGGGACAAGTGGTCAGATTCTAGATACATTTTGTAGGCAAAGTTGACAGGGTTTTCT
GATGGGGTGTTAAGAGAAAGAAGAGAGTCAAGGATGAAGCTAAGGTTTTTGGCTTCAGCAAGAG
GAAAAATCGAGTTGCCATTTGTTGACATGGAGAAGGCAATGAATGGAGCAAGTTTCGGTCAGGA
GATTTGGGAGGTGCCGGGTTTGTGATGCCTATCTGACATTCAAGTGGAGTGTTGATCTGGCAGT
TGAATAGAGAAGTGTGGAGTGCAGAGCAGAAGTACAGGATGGAGATGAAGATTTGGTGTCGAAA
GTGTAGAGGAATTGAAAATTATGGATCTGAAGACATCAACTAGTGAGTGGAGTAAATAGAGAAT
AGAGGTCCCAAGAGTGAGCCTTGGATCCTGCAGTGTTTCCAGGTTGCAGAGATAGAGGAGGAAC
TAACAAAGGAGACTGTAAAGGAGAGGTCAGGGAAGTAGGAAAAGAACTGGGAGAGGGTGGTGTT
TCAAATGAAGAAAGGGTTTGCTGGAAGAAGGGGTGATCAGATGTGTTGAGTGCTGCTAGTAAGT
CAAGTAAAATGAGGACAGGAGTTGATCAATGGATTTAGCAACATGGGGTTATTTGGGAGCTTGA
CAACAGTAGTTTTGGTGAAATGGCAGGGGTGAAAGCCTGACTGGAGTAGGTTTAAGAGAGAAGG
GGAGGAAAGGAATTGGAGGTAGAGGATAGAGATAACACTTTCAAGAATTTTGCTGCAAAGGAGA
GCAGAAAAATAAGGTGAGGTCAGTTCTAGCCTTGTCCACCCACCTTAAAAGTCACATTCCCACG
TGTCTCCACTCCTCCCATCCTCTTTCCCCTTTTCCCCAATCCATCTTTTCTTTTCTTTTCTTTT
CTTTTTTTTTTTTTTGAGACAGTATCTTGCTCTGTCGCCCAGGCTGGAGTGCAGTGGTGCAATC
ACAGCGCACTGCAGCCTCGACCTCTCCGGCTCAAGTGATCTTCCTGCCTCAGCCTCCTAAGTAG
CTGGGACCACAGGCATGAGCTACCATGTCCAGCTAATTTTTAAAAAATTATTTTTGTAGAGAGG
AGGGCTTGCTATGTTGCCAAGTCTGTCTCGAATTCCTGGGCTCAAGTGATACCTCCCTACCTAG
CCTCCCCAAATGCTGGGATTATAGGCATGCGCCACTGCGCTGGGCCTCCTAGTCCACTATTTCC
ATTCCTCTTTCACCAGACGGTTGTTTTACTTGTCAGGTTCGTGTGTGGTGCCTTTGCTTGGCCA
GTGGAGAACCACCAGAGCAGGGGCCAGTCCCTCCACCCTCAACTCAGCCTCCATCTGGCAGGGC
AATGGCCCCTTCCCTCAGATTGCCTGTCCCCTCGTGGTGCCTTCTCCTTCATCAGGCAACCTAT
AATTCTTGTGGAAGGGAGGGACGGCGTGTCCCCGGGCCCTCTGATGGTTCCCTTACCCTTAGGG
TGGACCAAATTGTGGGTCGGGGGCCCGGGGACAGGAAGGCCCGGGAGAAGGGCGACAAGGGGCC
CTCCGACGCGGAGGTGGTGGATGAAATCAGCATGATGGGACGCGTGGTCAAGGTGGAGAAGCAG
GTGAGTGTAGGATGGGGTGGCGGAGCTGGCCATACCAAGAAGCTCTGGGGGCCGCGGTGACATG
GGGAGGATGCGTTTCCCACAGCCACAGCCCAAGGGTCCCCGAGAAGACCTAAGAGCCCCCTCCC
CGGCCGAAGCCCCCGCCCTCGCCGCCAGACATTCCCATAAACCCGCCCTCTCCTGCTAAGCCCC
GCCCCCAGGCTGCATAAGCCCGCCCATAACCTTCAGATAAGCCTACCTCAGTTCCCCCTACTCC
ATTTGACCCCGCCTCTCAGTTGTCGCTGTAGCTCGCGGACTATCCCCATCGGTCACACCTAACC
ACCCCCCGGGTTATGCACCAGTGCCCAGCTTCGCCCCTCGCTCTAGCCAAGCTCCACCTTTCCA
GGCGGGATTTGTGCTTCCCAGATAAGCCCCGCCCACTCCACCGGCTAGGGTCCGCCCCGAGACC
CAAGCCCCGCCCCCGGCCGCGTCCCCTCCGGTCCCAGGCCCTGCTTCCCAGCTGCGCCCCGTCC
CCAGGTGCAGTCCATCGAGCACAAGCTGGACCTGCTGTTGGGCTTCTATTCGCGCTGCCTGCGC
TCTGGCACCTCGGCCAGCCTGGGCGCCGTGCAAGTGCCGCTGTTCGACCCCGACATCACCTCCG
ACTACCACAGCCCTGTGGACCACGAGGACATCTCCGTCTCCGCACAGACGCTCAGCATCTCCCG
CTCGGTCAGCACCAACATGGACTGAGGGACTTCTCAGAGGCAGGGCAGCACACGGCCAGCCCCG
CGGCCTGGCGCTCCGACTGCCCTCTGAGGCCTCCGGACTCCTCTCGTACTTGAACTCACTCCCT
CACGGGGAGAGAGACCACACGCAGTATTGAGCTGCCTGAGTGGGCGTGGTACCTGCTGTGGGTG
CCAGCGCCCCTTCCCCACCTCAGGAGCGTGAGATGCCAGGTCGCACAGAGGGCAGCAGCAGCGG
CCGTCCCGCGGCCTCTGGGCCCCCCAGTGCCCTGCCCACTCCATCAAGGCCCTATGTGGCCCAC
CTGGCAGGGGCACAGCCCCGGGAGTGGGAGCGGGCGCTGGGGCCCTGGGCCCTGACCCAGCTTC
CAGCTATGCAAGGTGAGGTCTCTGGCCCACCCTTCGGACACAGCAGGGAAGCCCTCCCGCCAAG
TCCCCGCCCCACTTGGGGGTGGGCCAAGGTGCCCCCACAGGTACCCACAAAGCACAGGACCCTG
CCACAAGGCAGGTGGACACCATATATGCAAACCATGTTAAATATGCAACTTTGGGGACCCCCAT
GGGGTCTCTCTGTCCCTCCCCCATTGGGAGCTGGGCCCCCAGCAGTAGCTGGTCTCAGGCTGCT
TGGCCACCACCCTGTCCCTATTCTTTGGCTTATCACTCCTTCCCCTCCCAGCATGGGGCCTGTT
TCTCCCCTGCCCTCTCCTAAGGGCAATGCCTGGGCCTTTCTTCCCATTTGCAAGTGTCAGCTCC
CAGGGGCTCCCTCCTCCTGCTGGGTGGCCACTCCCCTCCTTGGCCCTCCAGACACCACTCATAG
TCAGCACAGGTTTCTGTATCCTCCCCAAAACTCCCAGACAGTGCTTCGTGGACGATCGCACAAA
CATAGCCTTTTAGTTTCTCCAGACAGGAAGAAAGCCTCTCACACTTAAACATGCAATGAGGTGA
CACACTTGGAGACATGAGTGCAGAGCCACTCAGCCGCTCCTGGGCCTCTGCAGCAGATGCCAGT
GGACTGGCCTTGCAGGGTGACGACCACTAAGAGGAAGACCCCCAACTCCATCTGAGCAGGAGAA
GGAGCTTTGAAGTAACCCGAGAGCTCTCCAGGCCCCACCCAGACCTTTACCCGCTCCCCTTCTT
CAAGAAGATCTCCTCCTCTCTGGTCCAGGAGCCCTAACCCACTGCCTCTGCCTGTCCCCAAGGG
CCCGCCTCCGTGTCTCCACAGCACAACTCGGGCCCAGGCCTGACACCACTGGAGAGACCCCAGG
CCCACTTCTAGCCAGGCCTGTGCCTTCCTAGTCACTCTAACTCCCAGAGAGAATAAGAATGCAT
GTAATAGCTATACCAACCGCGCATCCGGCTTTCACATGCACTGTCTCCCCTCCCTCCACACCCC
ACTTCTTCACTTCAATTGGCAGCGCCACATCCAGGCGTCAGCCCCCATTCACTCCAGGAACACT
TTCTTATCCCCACCCCTTTGCTCCTCTTCTGCAAAGCCAATGCAGGTGGCAGGAAGGTGAGGGG
TAGTGGACCAATGGCAACCCTCTGTGGGAACAAGGGGCCGAGGCCACGCTGCCTGCATCTCGTG
CTGGGGACCTGCATGCGCCAGCACCAGGGCTTGGACTGGATCTTACTCAGTCCATGGTGCCCAG
CCTCTGCCCCAACATGCCCTCTGCATGTGACCGTCATGCCCTGGATGGAGCCACTCCTGGCTCA
CCCCACCTGCACTGCACTGTCCCCAGAGAGCCACCCCTCCACCCACTCAGAGACAGCTGTGGAG
AGGGCCAGGAGAATGGGATTACCCTATGACCAAGGAGACATGGGAAGAAGCCCTCCTTCCTTCC
ACGATCGAGGTTCCGCCATCAACTCGGTTCTCGGATATGCAAGTACCTCACTTTGTTAACTTAT
TAACTTATTGGTTTCATTAAAGTTTTCAAGAGGAGGTGATTGTTCTTGGTTTTGTTCAGTGGCT
GTGAGGTGAGGCTCCTGGTTTGGGGGTGGGGACAAGGAGATTTGGATTGGTCGGGACTCTTTGT
TGTATACAACAAAAACCCAACCCTGTATGGTTTAAACAGACAAACAAAAAGAAACTTATTGGCA
CAGAGAACTGAGAAGTCCAGGGGATAGATTTGGTTTCATGCACAGTGAGATCTAGATGCTTAAA
CGTGTCAGGACTCTTGTTTCCTGTAAGCCTTCCTTTCTGGCAGGCTGACCTCTCATGGTGACCA
GTAAAATTGGCCACCACTTTCCTAATAGTTTGCATCAGTCAAGATCAAGTCAGGAAGGCAGAAA
CAACACTAAGTTTTTCAGCAAAAGAATTTATTATAGGTAATGGGTTACACTGATGCAGGAGGGC
TGAAAGAACAAAAGAGAATTTTCTCTAGGTGATTCTGAGATAGCCTAGAGAAAATAACTGCAGA
AAGCAGTTACCACTCCCAGAGCTGCAAGAGTATAGGAAACAGGTTGGGGTTATCAGAACCCACA
AGCTTGCGGAGAGGGAGCCCGGTAGTGTTGGGGCTCAGAGATCTGAAAAGGAGGTACTCCCTGG
CTGGTGCTAGTACCTCTTAGGGGACACAATGAGGCTTGTCTGGGAGTATGGGAAAAAAGCCGAA
GTCTGGGATCAACTGTTGCAACCTGGCTGTTGCTGATAAGGATAGGAAGCAAAGGAAGAGAGAA
GTCTCCACTTTTCTTGCCTTCCAGTCTTCCCCTAGTACCCCTTATTGTCAAAGCTAGCGAACAA
AGGAGTGATGTGGTTTGCAGAGTCCCAGCCCCAGTATCACAGAGTTAGAAGGCTGCACTTGGAA
CTGAAAGACAATAACATGGGCACGGGCCACCCCTTTGGACACACAGTTTCCACAAACATCAATC
CATACATACTTGAACCGCCATATAACGACAGTAGCTTCCACCTAGTAAGATGAAACTGTCCTTT
ATACAAATGAAGACACTCTAGTCCCCTCCCCCTAAATGGGAAAAACACAACGTCCCACGGTTCA
ACAGATGCATCCATCTCCAGGGGTTAGTCACTGAATCCATATTTGGGTTAAGTATTCCTCTAAT
TCAGGCACAATTCCACCTAGAACTAAACACTAAAGTAAAGTTGATCATCACATACAATGTTTAT
ATAAAATATGGGAGAAAGGAGGAGGGAGGAAAAAATAATATGTACAGTGCACACACACACATGC
ACACACATCCCAAGCAAGGAAGGAAGTACGCATAGCCGCAATCGACCTCTTTCTGGTATCTCTG
GTCACAGTGCCATGGTTGGTATGAATAATTTCCTTATTCCATTGCCCTTCCATGTTTCCTTTGC
CCTCAGCCAACACCTCAGCTTTTTAGGGACCTTTTTTTTTTTTTTTTTTTGAGACAGAGTCTCG
CTTGGTTGCCAGGCTGGAATGTAGCGGTGCGATCTCAGCTCACTGCAACTTCCACCTCCTGGGT
TCAAGCGATTCTCCTGCCTTAGCCTCCCAAGTAGCTGGGACTACAGGCATGTGCCACCACACCC
AGCTAATTTTTGTATTTTTAGTAGAGATGGGGTTTCACCATGTTGGCCAGGATGGTCTTGATCT
CCTGATCTCGTGATCCACCTGCCTTGTCCTCCCAAAGTGCTGGGATTACAGGCGTGAGCCACCG
TGCCTGGCCAGGGATCTTTATATGGTGGAGTGACCCAGATAATCATTCCTGAAGGGTCTGAATC
CTCAGCGGTCCTGCATGGGTGGGTGTCACAGCTTTTCATTGACTTTTGCTATCATACATGGAAG
TACTTATTATAGAAAGGCCTTAGAGAATCCCTTGGGTTCCAGACTTAGTCCTCCTTGCCCTGTT
GTGTGGCAGCAACCAATTTCCGCTCGGTAATGAAGATCAGTCATGCAGCCAGTGTATTAACTCT
TTATTTTTCCTGCTGGTTGAGTGGCATGAGGAGCCCACAATGGCCTGGTT
EXEMPLARY HUMAN KCNQ4 TRANSCRIPT VARIANT 1 cDNA
SEQUENCE INCLUDING UNTRANSLATED REGIONS
(SEQ ID NO: 6)
GACATGTGAGCGGCGCGCGCCGGTGGCAGGTGGAAAGGCGAGCGGCATGGAGCGCGTAATAAGA
GAGTTGGAGTCGGAAAGAGCAGCCCCAGTCGCCGGGGAAGCGGGAGGTCAGTGCGGGCTCCGGC
GGCCCCCAGGCTCCGAGCGCCCGCCCGCGGCCCCGGCCCGGCCCCTAGCCCCCGCCGCCCGCGC
CCGCCCCGGGTCGCCCCTCTGGCCCCGGGTCCGAGCCATGCGTCTCTGAGCGCCCCGAGCGCGC
CCCCGCCCCGGACCGTGCCCGGGCCCCGGCGCCCCCAGCCCGGCGCCGCCCATGGCCGAGGCCC
CCCCGCGCCGCCTCGGCCTGGGTCCCCCGCCCGGGGACGCCCCCCGCGCGGAGCTAGTGGCGCT
CACGGCCGTGCAGAGCGAACAGGGCGAGGCGGGCGGGGGCGGCTCCCCGCGCCGCCTCGGCCTC
CTGGGCAGCCCCCTGCCGCCGGGCGCGCCCCTCCCTGGGCCGGGCTCCGGCTCGGGCTCCGCCT
GCGGCCAGCGCTCCTCGGCCGCGCACAAGCGCTACCGCCGCCTGCAGAACTGGGTCTACAACGT
GCTGGAGCGGCCCCGCGGCTGGGCCTTCGTCTACCACGTCTTCATATTTTTGCTGGTCTTCAGC
TGCCTGGTGCTGTCTGTGCTGTCCACTATCCAGGAGCACCAGGAACTTGCCAACGAGTGTCTCC
TCATCTTGGAATTCGTGATGATCGTGGTTTTCGGCTTGGAGTACATCGTCCGGGTCTGGTCCGC
CGGATGCTGCTGCCGCTACCGAGGATGGCAGGGTCGCTTCCGCTTTGCCAGAAAGCCCTTCTGT
GTCATCGACTTCATCGTGTTCGTGGCCTCGGTGGCCGTCATCGCCGCGGGTACCCAGGGCAACA
TCTTCGCCACGTCCGCGCTGCGCAGCATGCGCTTCCTGCAGATCCTGCGCATGGTGCGCATGGA
CCGCCGCGGCGGCACCTGGAAGCTGCTGGGCTCAGTGGTCTACGCGCATAGCAAGGAGCTGATC
ACCGCCTGGTACATCGGGTTCCTGGTGCTCATCTTCGCCTCCTTCCTGGTCTACCTGGCTGAGA
AGGACGCCAACTCCGACTTCTCCTCCTACGCCGACTCGCTCTGGTGGGGGACGATTACATTGAC
AACCATCGGCTATGGTGACAAGACACCGCACACATGGCTGGGCAGGGTCCTGGCTGCTGGCTTC
GCCTTACTGGGCATCTCTTTCTTTGCCCTGCCTGCCGGCATCCTAGGCTCCGGCTTTGCCCTGA
AGGTCCAGGAGCAGCACCGGCAGAAGCACTTCGAGAAGCGGAGGATGCCGGCAGCCAACCTCAT
CCAGGCTGCCTGGCGCCTGTACTCCACCGATATGAGCCGGGCCTACCTGACAGCCACCTGGTAC
TACTATGACAGTATCCTCCCATCCTTCAGAGAGCTGGCCCTCTTGTTTGAGCACGTGCAACGGG
CCCGCAATGGGGGCCTACGGCCCCTGGAGGTGCGGCGGGCGCCGGTACCCGACGGAGCACCCTC
CCGTTACCCGCCCGTTGCCACCTGCCACCGGCCGGGCAGCACCTCCTTCTGCCCTGGGGAAAGC
AGCCGGATGGGCATCAAAGACCGCATCCGCATGGGCAGCTCCCAGCGGCGGACGGGTCCTTCCA
AGCAGCATCTGGCACCTCCAACAATGCCCACCTCCCCAAGCAGCGAGCAGGTGGGTGAGGCCAC
CAGCCCCACCAAGGTGCAAAAGAGCTGGAGCTTCAATGACCGCACCCGCTTCCGGGCATCTCTG
AGACTCAAACCCCGCACCTCTGCTGAGGATGCCCCCTCAGAGGAAGTAGCAGAGGAGAAGAGCT
ACCAGTGTGAGCTCACGGTGGACGACATCATGCCTGCTGTGAAGACAGTCATCCGCTCCATCAG
GATTCTCAAGTTCCTGGTGGCCAAAAGGAAATTCAAGGAGACACTGCGACCGTACGACGTGAAG
GACGTCATTGAGCAGTACTCAGCAGGCCACCTGGACATGCTGGGCCGGATCAAGAGCCTGCAAA
CTCGGGTGGACCAAATTGTGGGTCGGGGGCCCGGGGACAGGAAGGCCCGGGAGAAGGGCGACAA
GGGGCCCTCCGACGCGGAGGTGGTGGATGAAATCAGCATGATGGGACGCGTGGTCAAGGTGGAG
AAGCAGGTGCAGTCCATCGAGCACAAGCTGGACCTGCTGTTGGGCTTCTATTCGCGCTGCCTGC
GCTCTGGCACCTCGGCCAGCCTGGGCGCCGTGCAAGTGCCGCTGTTCGACCCCGACATCACCTC
CGACTACCACAGCCCTGTGGACCACGAGGACATCTCCGTCTCCGCACAGACGCTCAGCATCTCC
CGCTCGGTCAGCACCAACATGGACTGAGGGACTTCTCAGAGGCAGGGCAGCACACGGCCAGCCC
CGCGGCCTGGCGCTCCGACTGCCCTCTGAGGCCTCCGGACTCCTCTCGTACTTGAACTCACTCC
CTCACGGGGAGAGAGACCACACGCAGTATTGAGCTGCCTGAGTGGGCGTGGTACCTGCTGTGGG
TGCCAGCGCCCCTTCCCCACCTCAGGAGCGTGAGATGCCAGGTCGCACAGAGGGCAGCAGCAGC
GGCCGTCCCGCGGCCTCTGGGCCCCCCAGTGCCCTGCCCACTCCATCAAGGCCCTATGTGGCCC
ACCTGGCAGGGGCACAGCCCCGGGAGTGGGAGCGGGCGCTGGGGCCCTGGGCCCTGACCCAGCT
TCCAGCTATGCAAGGTGAGGTCTCTGGCCCACCCTTCGGACACAGCAGGGAAGCCCTCCCGCCA
AGTCCCCGCCCCACTTGGGGGTGGGCCAAGGTGCCCCCACAGGTACCCACAAAGCACAGGACCC
TGCCACAAGGCAGGTGGACACCATATATGCAAACCATGTTAAATATGCAACTTTGGGGACCCCC
ATGGGGTCTCTCTGTCCCTCCCCCATTGGGAGCTGGGCCCCCAGCAGTAGCTGGTCTCAGGCTG
CTTGGCCACCACCCTGTCCCTATTCTTTGGCTTATCACTCCTTCCCCTCCCAGCATGGGGCCTG
TTTCTCCCCTGCCCTCTCCTAAGGGCAATGCCTGGGCCTTTCTTCCCATTTGCAAGTGTCAGCT
CCCAGGGGCTCCCTCCTCCTGCTGGGTGGCCACTCCCCTCCTTGGCCCTCCAGACACCACTCAT
AGTCAGCACAGGTTTCTGTATCCTCCCCAAAACTCCCAGACAGTGCTTCGTGGACGATCGCACA
AACATAGCCTTTTAGTTTCTCCAGACAGGAAGAAAGCCTCTCACACTTAAACATGCAATGAGGT
GACACACTTGGAGACATGAGTGCAGAGCCACTCAGCCGCTCCTGGGCCTCTGCAGCAGATGCCA
GTGGACTGGCCTTGCAGGGTGACGACCACTAAGAGGAAGACCCCCAACTCCATCTGAGCAGGAG
AAGGAGCTTTGAAGTAACCCGAGAGCTCTCCAGGCCCCACCCAGACCTTTACCCGCTCCCCTTC
TTCAAGAAGATCTCCTCCTCTCTGGTCCAGGAGCCCTAACCCACTGCCTCTGCCTGTCCCCAAG
GGCCCGCCTCCGTGTCTCCACAGCACAACTCGGGCCCAGGCCTGACACCACTGGAGAGACCCCA
GGCCCACTTCTAGCCAGGCCTGTGCCTTCCTAGTCACTCTAACTCCCAGAGAGAATAAGAATGC
ATGTAATAGCTATACCAACCGCGCATCCGGCTTTCACATGCACTGTCTCCCCTCCCTCCACACC
CCACTTCTTCACTTCAATTGGCAGCGCCACATCCAGGCGTCAGCCCCCATTCACTCCAGGAACA
CTTTCTTATCCCCACCCCTTTGCTCCTCTTCTGCAAAGCCAATGCAGGTGGCAGGAAGGTGAGG
GGTAGTGGACCAATGGCAACCCTCTGTGGGAACAAGGGGCCGAGGCCACGCTGCCTGCATCTCG
TGCTGGGGACCTGCATGCGCCAGCACCAGGGCTTGGACTGGATCTTACTCAGTCCATGGTGCCC
AGCCTCTGCCCCAACATGCCCTCTGCATGTGACCGTCATGCCCTGGATGGAGCCACTCCTGGCT
CACCCCACCTGCACTGCACTGTCCCCAGAGAGCCACCCCTCCACCCACTCAGAGACAGCTGTGG
AGAGGGCCAGGAGAATGGGATTACCCTATGACCAAGGAGACATGGGAAGAAGCCCTCCTTCCTT
CCACGATCGAGGTTCCGCCATCAACTCGGTTCTCGGATATGCAAGTACCTCACTTTGTTAACTT
ATTAACTTATTGGTTTCATTAAAGTTTTCAAGAGGA
EXEMPLARY HUMAN KCNQ4 TRANSCRIPT VARIANT 2 cDNA
SEQUENCE INCLUDING UNTRANSLATED REGIONS
(SEQ ID NO: 7)
GACATGTGAGCGGCGCGCGCCGGTGGCAGGTGGAAAGGCGAGCGGCATGGAGCGCGTAATAAGA
GAGTTGGAGTCGGAAAGAGCAGCCCCAGTCGCCGGGGAAGCGGGAGGTCAGTGCGGGCTCCGGC
GGCCCCCAGGCTCCGAGCGCCCGCCCGCGGCCCCGGCCCGGCCCCTAGCCCCCGCCGCCCGCGC
CCGCCCCGGGTCGCCCCTCTGGCCCCGGGTCCGAGCCATGCGTCTCTGAGCGCCCCGAGCGCGC
CCCCGCCCCGGACCGTGCCCGGGCCCCGGCGCCCCCAGCCCGGCGCCGCCCATGGCCGAGGCCC
CCCCGCGCCGCCTCGGCCTGGGTCCCCCGCCCGGGGACGCCCCCCGCGCGGAGCTAGTGGCGCT
CACGGCCGTGCAGAGCGAACAGGGCGAGGCGGGCGGGGGCGGCTCCCCGCGCCGCCTCGGCCTC
CTGGGCAGCCCCCTGCCGCCGGGCGCGCCCCTCCCTGGGCCGGGCTCCGGCTCGGGCTCCGCCT
GCGGCCAGCGCTCCTCGGCCGCGCACAAGCGCTACCGCCGCCTGCAGAACTGGGTCTACAACGT
GCTGGAGCGGCCCCGCGGCTGGGCCTTCGTCTACCACGTCTTCATATTTTTGCTGGTCTTCAGC
TGCCTGGTGCTGTCTGTGCTGTCCACTATCCAGGAGCACCAGGAACTTGCCAACGAGTGTCTCC
TCATCTTGGAATTCGTGATGATCGTGGTTTTCGGCTTGGAGTACATCGTCCGGGTCTGGTCCGC
CGGATGCTGCTGCCGCTACCGAGGATGGCAGGGTCGCTTCCGCTTTGCCAGAAAGCCCTTCTGT
GTCATCGACTTCATCGTGTTCGTGGCCTCGGTGGCCGTCATCGCCGCGGGTACCCAGGGCAACA
TCTTCGCCACGTCCGCGCTGCGCAGCATGCGCTTCCTGCAGATCCTGCGCATGGTGCGCATGGA
CCGCCGCGGCGGCACCTGGAAGCTGCTGGGCTCAGTGGTCTACGCGCATAGCAAGGAGCTGATC
ACCGCCTGGTACATCGGGTTCCTGGTGCTCATCTTCGCCTCCTTCCTGGTCTACCTGGCTGAGA
AGGACGCCAACTCCGACTTCTCCTCCTACGCCGACTCGCTCTGGTGGGGGACGATTACATTGAC
AACCATCGGCTATGGTGACAAGACACCGCACACATGGCTGGGCAGGGTCCTGGCTGCTGGCTTC
GCCTTACTGGGCATCTCTTTCTTTGCCCTGCCTGCCGGCATCCTAGGCTCCGGCTTTGCCCTGA
AGGTCCAGGAGCAGCACCGGCAGAAGCACTTCGAGAAGCGGAGGATGCCGGCAGCCAACCTCAT
CCAGGCTGCCTGGCGCCTGTACTCCACCGATATGAGCCGGGCCTACCTGACAGCCACCTGGTAC
TACTATGACAGTATCCTCCCATCCTTCAGCAGCCGGATGGGCATCAAAGACCGCATCCGCATGG
GCAGCTCCCAGCGGCGGACGGGTCCTTCCAAGCAGCATCTGGCACCTCCAACAATGCCCACCTC
CCCAAGCAGCGAGCAGGTGGGTGAGGCCACCAGCCCCACCAAGGTGCAAAAGAGCTGGAGCTTC
AATGACCGCACCCGCTTCCGGGCATCTCTGAGACTCAAACCCCGCACCTCTGCTGAGGATGCCC
CCTCAGAGGAAGTAGCAGAGGAGAAGAGCTACCAGTGTGAGCTCACGGTGGACGACATCATGCC
TGCTGTGAAGACAGTCATCCGCTCCATCAGGATTCTCAAGTTCCTGGTGGCCAAAAGGAAATTC
AAGGAGACACTGCGACCGTACGACGTGAAGGACGTCATTGAGCAGTACTCAGCAGGCCACCTGG
ACATGCTGGGCCGGATCAAGAGCCTGCAAACTCGGGTGGACCAAATTGTGGGTCGGGGGCCCGG
GGACAGGAAGGCCCGGGAGAAGGGCGACAAGGGGCCCTCCGACGCGGAGGTGGTGGATGAAATC
AGCATGATGGGACGCGTGGTCAAGGTGGAGAAGCAGGTGCAGTCCATCGAGCACAAGCTGGACC
TGCTGTTGGGCTTCTATTCGCGCTGCCTGCGCTCTGGCACCTCGGCCAGCCTGGGCGCCGTGCA
AGTGCCGCTGTTCGACCCCGACATCACCTCCGACTACCACAGCCCTGTGGACCACGAGGACATC
TCCGTCTCCGCACAGACGCTCAGCATCTCCCGCTCGGTCAGCACCAACATGGACTGAGGGACTT
CTCAGAGGCAGGGCAGCACACGGCCAGCCCCGCGGCCTGGCGCTCCGACTGCCCTCTGAGGCCT
CCGGACTCCTCTCGTACTTGAACTCACTCCCTCACGGGGAGAGAGACCACACGCAGTATTGAGC
TGCCTGAGTGGGCGTGGTACCTGCTGTGGGTGCCAGCGCCCCTTCCCCACCTCAGGAGCGTGAG
ATGCCAGGTCGCACAGAGGGCAGCAGCAGCGGCCGTCCCGCGGCCTCTGGGCCCCCCAGTGCCC
TGCCCACTCCATCAAGGCCCTATGTGGCCCACCTGGCAGGGGCACAGCCCCGGGAGTGGGAGCG
GGCGCTGGGGCCCTGGGCCCTGACCCAGCTTCCAGCTATGCAAGGTGAGGTCTCTGGCCCACCC
TTCGGACACAGCAGGGAAGCCCTCCCGCCAAGTCCCCGCCCCACTTGGGGGTGGGCCAAGGTGC
CCCCACAGGTACCCACAAAGCACAGGACCCTGCCACAAGGCAGGTGGACACCATATATGCAAAC
CATGTTAAATATGCAACTTTGGGGACCCCCATGGGGTCTCTCTGTCCCTCCCCCATTGGGAGCT
GGGCCCCCAGCAGTAGCTGGTCTCAGGCTGCTTGGCCACCACCCTGTCCCTATTCTTTGGCTTA
TCACTCCTTCCCCTCCCAGCATGGGGCCTGTTTCTCCCCTGCCCTCTCCTAAGGGCAATGCCTG
GGCCTTTCTTCCCATTTGCAAGTGTCAGCTCCCAGGGGCTCCCTCCTCCTGCTGGGTGGCCACT
CCCCTCCTTGGCCCTCCAGACACCACTCATAGTCAGCACAGGTTTCTGTATCCTCCCCAAAACT
CCCAGACAGTGCTTCGTGGACGATCGCACAAACATAGCCTTTTAGTTTCTCCAGACAGGAAGAA
AGCCTCTCACACTTAAACATGCAATGACGTGACACACTTGGAGACATGAGTGCAGAGCCACTCA
GCCGCTCCTGGGCCTCTGCAGCAGATGCCAGTGGACTGGCCTTGCAGGGTGACGACCACTAAGA
GGAAGACCCCCAACTCCATCTGAGCAGGAGAAGGAGCTTTGAAGTAACCCGAGAGCTCTCCAGG
CCCCACCCAGACCTTTACCCGCTCCCCTTCTTCAAGAAGATCTCCTCCTCTCTGGTCCAGGAGC
CCTAACCCACTGCCTCTGCCTGTCCCCAAGGGCCCGCCTCCGTGTCTCCACAGCACAACTCGGG
CCCAGGCCTGACACCACTGGAGAGACCCCAGGCCCACTTCTAGCCAGGCCTGTGCCTTCCTAGT
CACTCTAACTCCCAGAGAGAATAAGAATGCATGTAATAGCTATACCAACCGCGCATCCGGCTTT
CACATGCACTGTCTCCCCTCCCTCCACACCCCACTTCTTCACTTCAATTGGCAGCGCCACATCC
AGGCGTCAGCCCCCATTCACTCCAGGAACACTTTCTTATCCCCACCCCTTTGCTCCTCTTCTGC
AAAGCCAATGCAGGTGGCAGGAAGGTGAGGGGTAGTGGACCAATGGCAACCCTCTGTGGGAACA
AGGGGCCGAGGCCACGCTGCCTGCATCTCGTGCTGGGGACCTGCATGCGCCAGCACCAGGGCTT
GGACTGGATCTTACTCAGTCCATGGTGCCCAGCCTCTGCCCCAACATGCCCTCTGCATGTGACC
GTCATGCCCTGGATGGAGCCACTCCTGGCTCACCCCACCTGCACTGCACTGTCCCCAGAGAGCC
ACCCCTCCACCCACTCAGAGACAGCTGTGGAGAGGGCCAGGAGAATGGGATTACCCTATGACCA
AGGAGACATGGGAAGAAGCCCTCCTTCCTTCCACGATCGAGGTTCCGCCATCAACTCGGTTCTC
GGATATGCAAGTACCTCACTTTGTTAACTTATTAACTTATTGGTTTCATTAAAGTTTTCAAGAG
GA
EXEMPLARY HUMAN KCNQ4 TRANSCRIPT X1 cDNA SEQUENCE
INCLUDING UNTRANSLATED REGIONS
(SEQ ID NO: 8)
GACCACTCGTACCACAAAGTGGGCAGCTGAGGAGCTGATCACCGCCTGGTACATCGGGTTCCTG
GTGCTCATCTTCGCCTCCTTCCTGGTCTACCTGGCTGAGAAGGACGCCAACTCCGACTTCTCCT
CCTACGCCGACTCGCTCTGGTGGGGGACGATTACATTGACAACCATCGGCTATGGTGACAAGAC
ACCGCACACATGGCTGGGCAGGGTCCTGGCTGCTGGCTTCGCCTTACTGGGCATCTCTTTCTTT
GCCCTGCCTGCCGGCATCCTAGGCTCCGGCTTTGCCCTGAAGGTCCAGGAGCAGCACCGGCAGA
AGCACTTCGAGAAGCGGAGGATGCCGGCAGCCAACCTCATCCAGGCTGCCTGGCGCCTGTACTC
CACCGATATGAGCCGGGCCTACCTGACAGCCACCTGGTACTACTATGACAGTATCCTCCCATCC
TTCAGAGAGCTGGCCCTCTTGTTTGAGCACGTGCAACGGGCCCGCAATGGGGGCCTACGGCCCC
TGGAGGTGCGGCGGGCGCCGGTACCCGACGGAGCACCCTCCCGTTACCCGCCCGTTGCCACCTG
CCACCGGCCGGGCAGCACCTCCTTCTGCCCTGGGGAAAGCAGCCGGATGGGCATCAAAGACCGC
ATCCGCATGGGCAGCTCCCAGCGGCGGACGGGTCCTTCCAAGCAGCATCTGGCACCTCCAACAA
TGCCCACCTCCCCAAGCAGCGAGCAGGTGGGTGAGGCCACCAGCCCCACCAAGGTGCAAAAGAG
CTGGAGCTTCAATGACCGCACCCGCTTCCGGGCATCTCTGAGACTCAAACCCCGCACCTCTGCT
GAGGATGCCCCCTCAGAGGAAGTAGCAGAGGAGAAGAGCTACCAGTGTGAGCTCACGGTGGACG
ACATCATGCCTGCTGTGAAGACAGTCATCCGCTCCATCAGGATTCTCAAGTTCCTGGTGGCCAA
AAGGAAATTCAAGGAGACACTGCGACCGTACGACGTGAAGGACGTCATTGAGCAGTACTCAGCA
GGCCACCTGGACATGCTGGGCCGGATCAAGAGCCTGCAAACTCGGGTGGACCAAATTGTGGGTC
GGGGGCCCGGGGACAGGAAGGCCCGGGAGAAGGGCGACAAGGGGCCCTCCGACGCGGAGGTGGT
GGATGAAATCAGCATGATGGGACGCGTGGTCAAGGTGGAGAAGCAGGTGCAGTCCATCGAGCAC
AAGCTGGACCTGCTGTTGGGCTTCTATTCGCGCTGCCTGCGCTCTGGCACCTCGGCCAGCCTGG
GCGCCGTGCAAGTGCCGCTGTTCGACCCCGACATCACCTCCGACTACCACAGCCCTGTGGACCA
CGAGGACATCTCCGTCTCCGCACAGACGCTCAGCATCTCCCGCTCGGTCAGCACCAACATGGAC
TGAGGGACTTCTCAGAGGCAGGGCAGCACACGGCCAGCCCCGCGGCCTGGCGCTCCGACTGCCC
TCTGAGGCCTCCGGACTCCTCTCGTACTTGAACTCACTCCCTCACGGGGAGAGAGACCACACGC
AGTATTGAGCTGCCTGAGTGGGCGTGGTACCTGCTGTGGGTGCCAGCGCCCCTTCCCCACCTCA
GGAGCGTGAGATGCCAGGTCGCACAGAGGGCAGCAGCAGCGGCCGTCCCGCGGCCTCTGGGCCC
CCCAGTGCCCTGCCCACTCCATCAAGGCCCTATGTGGCCCACCTGGCAGGGGCACAGCCCCGGG
AGTGGGAGCGGGCGCTGGGGCCCTGGGCCCTGACCCAGCTTCCAGCTATGCAAGGTGAGGTCTC
TGGCCCACCCTTCGGACACAGCAGGGAAGCCCTCCCGCCAAGTCCCCGCCCCACTTGGGGGTGG
GCCAAGGTGCCCCCACAGGTACCCACAAAGCACAGGACCCTGCCACAAGGCAGGTGGACACCAT
ATATGCAAACCATGTTAAATATGCAACTTTGGGGACCCCCATGGGGTCTCTCTGTCCCTCCCCC
ATTGGGAGCTGGGCCCCCAGCAGTAGCTGGTCTCAGGCTGCTTGGCCACCACCCTGTCCCTATT
CTTTGGCTTATCACTCCTTCCCCTCCCAGCATGGGGCCTGTTTCTCCCCTGCCCTCTCCTAAGG
GCAATGCCTGGGCCTTTCTTCCCATTTGCAAGTGTCAGCTCCCAGGGGCTCCCTCCTCCTGCTG
GGTGGCCACTCCCCTCCTTGGCCCTCCAGACACCACTCATAGTCAGCACAGGTTTCTGTATCCT
CCCCAAAACTCCCAGACAGTGCTTCGTGGACGATCGCACAAACATAGCCTTTTAGTTTCTCCAG
ACAGGAAGAAAGCCTCTCACACTTAAACATGCAATGAGGTGACACACTTGGAGACATGAGTGCA
GAGCCACTCAGCCGCTCCTGGGCCTCTGCAGCAGATGCCAGTGGACTGGCCTTGCAGGGTGACG
ACCACTAAGAGGAAGACCCCCAACTCCATCTGAGCAGGAGAAGGAGCTTTGAAGTAACCCGAGA
GCTCTCCAGGCCCCACCCAGACCTTTACCCGCTCCCCTTCTTCAAGAAGATCTCCTCCTCTCTG
GTCCAGGAGCCCTAACCCACTGCCTCTGCCTGTCCCCAAGGGCCCGCCTCCGTGTCTCCACAGC
ACAACTCGGGCCCAGGCCTGACACCACTGGAGAGACCCCAGGCCCACTTCTAGCCAGGCCTGTG
CCTTCCTAGTCACTCTAACTCCCAGAGAGAATAAGAATGCATGTAATAGCTATACCAACCGCGC
ATCCGGCTTTCACATGCACTGTCTCCCCTCCCTCCACACCCCACTTCTTCACTTCAATTGGCAG
CGCCACATCCAGGCGTCAGCCCCCATTCACTCCAGGAACACTTTCTTATCCCCACCCCTTTGCT
CCTCTTCTGCAAAGCCAATGCAGGTGGCAGGAAGGTGAGGGGTAGTGGACCAATGGCAACCCTC
TGTGGGAACAAGGGGCCGAGGCCACGCTGCCTGCATCTCGTGCTGGGGACCTGCATGCGCCAGC
ACCAGGGCTTGGACTGGATCTTACTCAGTCCATGGTGCCCAGCCTCTGCCCCAACATGCCCTCT
GCATGTGACCGTCATGCCCTGGATGGAGCCACTCCTGGCTCACCCCACCTGCACTGCACTGTCC
CCAGAGAGCCACCCCTCCACCCACTCAGAGACAGCTGTGGAGAGGGCCAGGAGAATGGGATTAC
CCTATGACCAAGGAGACATGGGAAGAAGCCCTCCTTCCTTCCACGATCGAGGTTCCGCCATCAA
CTCGGTTCTCGGATATGCAAGTACCTCACTTTGTTAACTTATTAACTTATTGGTTTCATTAAAG
TTTTCAAGAGGAGGTGA
KCNQ4 (codon optimized; minimizes gRNA binding)
(SEQ ID NO: 9)
ATGGCTGAAGCCCCTCCTAGAAGGCTTGGACTGGGACCTCCTCCTGGGGATGCTCCTAGAGCTG
AACTGGTGGCTCTGACAGCCGTGCAGTCTGAACAAGGCGAAGCTGGTGGCGGCGGATCTCCACG
TAGACTTGGACTGCTGGGAAGCCCTCTTCCTCCTGGTGCTCCACTTCCTGGACCTGGCAGTGGA
TCTGGATCTGCCTGTGGCCAGAGAAGCTCTGCCGCTCACAAGAGATACCGGCGGCTGCAGAACT
GGGTGTACAACGTGCTGGAAAGACCCAGAGGCTGGGCCTTCGTGTACCACGTGTTCATCTTTCT
GCTGGTGTTCAGCTGCCTGGTGCTGTCCGTGCTGAGCACCATCCAAGAACATCAAGAGCTGGCT
AACGAGTGCCTGTTAATACTGGAGTTTGTGATGATTGTGGTGTTCGGCCTCGAGTACATCGTCC
GCGTTTGGAGCGCCGGCTGCTGCTGCAGATATAGAGGTTGGCAAGGCAGATTCCGCTTCGCCAG
AAAGCCCTTCTGCGTGATCGACTTCATCGTGTTCGTGGCCAGCGTGGCCGTGATTGCTGCTGGC
ACACAGGGCAACATCTTCGCCACAAGCGCCCTGCGGAGCATGCGGTTTCTGCAGATCCTGAGAA
TGGTCCGAATGGACAGAAGAGGCGGCACCTGGAAGCTGCTGGGCTCTGTGGTGTACGCCCACAG
CAAAGAGCTGATCACCGCCTGGTACATCGGATTTCTGGTGCTGATCTTCGCCTCCTTCCTGGTG
TACCTGGCCGAGAAGGACGCCAACAGCGACTTTAGCAGCTACGCCGACTCTCTTTGGTGGGGCA
CCATCACACTGACCACCATCGGCTACGGCGACAAGACCCCTCACACATGGCTGGGAAGAGTGCT
GGCCGCTGGATTTGCTCTGCTGGGCATCAGCTTTTTCGCCCTGCCTGCCGGAATCCTCGGATCT
GGCTTTGCCCTGAAGGTGCAAGAGCAGCACCGGCAGAAGCACTTCGAGAAGAGAAGAATGCCTG
CCGCCAACCTGATTCAGGCCGCTTGGAGACTGTACAGCACCGACATGAGCAGAGCCTACCTGAC
CGCCACGTGGTATTATTACGACTCGATCCTGCCTAGCTTCCGCGAACTGGCCCTGCTGTTTGAG
CATGTGCAGAGAGCCAGAAACGGCGGCCTCAGACCTCTGGAAGTTCGGAGAGCACCTGTGCCTG
ATGGCGCCCCTTCTAGATATCCTCCAGTGGCCACCTGTCACAGACCCGGCAGCACATCTTTTTG
CCCTGGCGAGTCTAGCCGGATGGGCATCAAGGACAGAATCAGAATGGGCAGCAGCCAGCGGAGA
ACAGGCCCTTCTAAACAGCATCTGGCCCCTCCAACCATGCCTACAAGCCCTAGCTCTGAGCAAG
TGGGCGAAGCCACCTCTCCTACCAAGGTGCAGAAGTCCTGGTCCTTCAACGACCGGACCAGATT
CAGAGCCAGCCTGAGACTGAAGCCCAGAACCTCTGCCGAGGATGCCCCTTCTGAAGAGGTGGCC
GAAGAGAAGTCCTACCAGTGCGAGCTGACCGTGGACGACATCATGCCAGCCGTGAAAACCGTGA
TACGGTCTATCCGGATCCTGAAGTTCCTGGTGGCCAAGCGGAAGTTCAAAGAGACACTGCGGCC
CTACGACGTGAAGGACGTGATCGAGCAGTATTCTGCCGGCCACCTGGACATGCTGGGCAGAATC
AAGAGCCTGCAGACCAGAGTGGACCAGATCGTTGGAAGAGGCCCAGGCGACAGAAAGGCCAGAG
AGAAGGGCGATAAGGGCCCATCTGATGCCGAGGTTGTCGACGAGATATCAATGATGGGCAGAGT
GGTCAAGGTGGAAAAACAGGTGCAGAGCATCGAGCACAAGCTGGACCTGCTGCTGGGATTCTAC
AGCCGGTGTCTGAGAAGCGGCACATCTGCATCTCTGGGCGCTGTGCAGGTCCCACTGTTCGACC
CTGATATCACCAGCGACTATCACAGCCCCGTGGACCACGAGGACATCTCCGTTTCTGCTCAGAC
CCTGAGCATGAGCAGATCCGTGTCCACCAACATGGAG
KCNQ4 (codon-optimized to resist miRNA-mediated degradation)
(SEQ ID NO: 10)
ATGGCTGAAGCCCCTCCTAGAAGGCTTGGACTGGGACCTCCTCCTGGGGATGCTCCTAGAGCTG
AACTGGTGGCTCTGACAGCCGTGCAGTCTGAACAAGGCGAAGCTGGTGGCGGCGGATCTCCACG
TAGACTTGGACTGCTGGGAAGCCCTCTTCCTCCTGGTGCTCCACTTCCTGGACCTGGCAGTGGA
TCTGGATCTGCCTGTGGCCAGAGAAGCTCTGCCGCTCACAAGAGATACCGGCGGCTGCAGAACT
GGGTGTACAACGTGCTGGAAAGACCCAGAGGCTGGGCCTTCGTGTACCACGTGTTCATCTTTCT
GCTGGTGTTCAGCTGCCTGGTGCTGTCCGTGCTGAGCACCATCCAAGAACATCAAGAGCTGGCT
AACGAGTGCCTGTTAATACTGGAGTTTGTGATGATTGTGGTGTTCGGCCTCGAGTACATCGTCC
GCGTTTGGAGCGCCGGCTGCTGCTGCAGATATAGAGGTTGGCAAGGCAGATTCCGCTTCGCCAG
AAAGCCCTTCTGCGTGATCGACTTCATCGTGTTCGTGGCCAGCGTGGCCGTGATTGCTGCTGGC
ACACAGGGCAACATCTTCGCCACAAGCGCCCTGCGGAGCATGCGGTTTCTGCAGATCCTGAGAA
TGGTCCGAATGGACAGAAGAGGCGGCACCTGGAAGCTGCTGGGCTCTGTGGTGTACGCCCACAG
CAAAGAGCTGATCACCGCCTGGTACATCGGATTTCTGGTGCTGATCTTCGCCTCCTTCCTGGTG
TACCTGGCCGAGAAGGACGCCAACAGCGACTTTAGCAGCTACGCCGACTCTCTTTGGTGGGGCA
CCATCACACTGACCACCATCGGCTACGGCGACAAGACCCCTCACACATGGCTGGGAAGAGTGCT
GGCCGCTGGATTTGCTCTGCTGGGCATCAGCTTTTTCGCCCTGCCTGCCGGAATCCTCGGATCT
GGCTTTGCCCTGAAGGTGCAAGAGCAGCATAGACAAAAGCATTTTGAAAAGAGAAGAATGCCTG
CCGCCAACCTGATTCAGGCCGCTTGGAGACTGTACAGCACCGACATGAGCAGAGCCTACCTGAC
CGCCACGTGGTATTATTACGATTCGATCCTGCCTAGCTTCCGCGAACTGGCCCTGCTGTTTGAG
CATGTGCAGAGAGCCAGAAACGGCGGCCTCAGACCTCTGGAAGTTCGGAGAGCACCTGTGCCTG
ATGGCGCCCCTTCTAGATATCCTCCAGTGGCCACCTGTCACAGACCCGGCAGCACATCTTTTTG
CCCTGGCGAGTCTAGCCGGATGGGCATCAAGGACAGAATCAGAATGGGCAGCAGCCAGCGGAGA
ACAGGCCCTTCTAAACAGCATCTGGCCCCTCCAACCATGCCTACAAGCCCTAGCTCTGAGCAAG
TGGGCGAAGCCACCTCTCCTACCAAGGTGCAGAAGTCCTGGTCCTTCAACGACCGGACCAGATT
TAGAGCCAGCCTCCGGCTGAAGCCCAGAACCTCTGCCGAGGATGCCCCTTCTGAAGAGGTGGCC
GAAGAGAAGTCCTACCAGTGCGAGCTGACCGTGGACGACATCATGCCAGCCGTGAAAACCGTGA
TAAGGTCTATACGCATCCTGAAGTTCCTGGTGGCCAAGCGGAAGTTCAAAGAGACACTGCGGCC
CTACGACGTGAAGGACGTGATCGAGCAGTATTCTGCCGGCCACCTGGACATGCTGGGCAGAATC
AAGAGCCTGCAGACCAGAGTGGACCAGATCGTTGGAAGAGGCCCAGGCGACAGAAAGGCCAGAG
AGAAGGGCGATAAGGGCCCATCTGATGCCGAGGTTGTCGACGAGATATCAATGATGGGCAGAGT
CGTGAAAGTCGAAAAACAGGTGCAGAGCATCGAGCACAAGCTGGACCTGCTGCTGGGATTCTAC
AGCCGGTGTCTGAGAAGCGGCACATCTGCATCTCTGGGCGCTGTGCAGGTCCCACTGTTCGATC
CTGATATTACGAGCGATTATCACAGCCCCGTGGACCACGAGGACATCTCCGTTTCTGCTCAGAC
CCTGAGCATCAGCAGATCCGTGTCCACCAACATGGAC
EXEMPLARY MOUSE KCNQ4 MEMBER 4 cDNA CODING SEQUENCE
(SEQ ID NO: 91)
ATGGCCGAGGCCCCCCCGCGCCGCCTCGGCCTGGGCCCCCCGCCCGGGGACGCCCCCCGCGCGG
AGTTGGTGGCGCTCACGGCCGTGCAGAGTGAACAGGGCGAGGCGGGCGGGGGCGGCTCTCCGCG
TCGCCTCGGCCTTCTGGGCAGCCCCCTGCCGCCGGGCGCGCCCCTCCCTGGGCCGGGCTCCGGC
TCGGGCTCCGCCTGCGGCGGCCAGCGCTCCTCCGCCGCGCAGAAGCGCTACCGCCGCCTGCAGA
ACTGGGTCTACAACGTGCTGGAGCGGCCCCGCGGGTGGGCCTTCGTCTACCACGTCTTCATATT
TTTGCTAGTCTTCAGCTGCCTGGTGCTGTCTGTACTGTCCACCATCCAGGAGCACCAGGAACTT
GCCAACGAGTGTCTCCTTATCTTGGAATTCGTGATGATTGTGGTCTTTGGCTTGGAGTATATAA
TCCGTGTCTGGTCGGCCGGATGCTGTTGTCGCTACAGAGGATGGCAGGGACGCTTTCGCTTCGC
CAGGAAACCCTTCTGTGTCATCGACTTCATCGTGTTCGTGGCCTCGGTGGCAGTGATAGCTGCG
GGCACACAAGGCAACATCTTTGCTACGTCCGCGTTGCGCAGTATGCGCTTCCTACAGATCCTGC
GCATGGTGCGTATGGATCGCCGCGGTGGCACCTGGAAGCTGTTGGGATCCGTGGTCTATGCGCA
CAGTAAGGAGCTGATCACCGCCTGGTACATCGGGTTCCTGGTGCTCATCTTTGCCTCTTTCCTG
GTCTACCTGGCTGAGAAGGATGCCAACTCTGACTTCTCCTCCTATGCCGACTCGCTCTGGTGGG
GGACGATCACACTGACGACCATTGGCTATGGTGACAAGACGCCACATACATGGCTGGGCAGGGT
TCTGGCTGCCGGCTTCGCCTTACTGGGCATCTCCTTCTTTGCCCTGCCTGCCGGCATCCTGGGC
TCTGGCTTTGCCCTGAAGGTCCAGGAGCAGCACAGGCAGAAGCACTTTGAGAAGCGCAGGATGC
CAGCAGCTAATCTCATCCAGGCTGCGTGGCGCCTGTACTCCACGGACACAAGCCGGGCCTATTT
GACAGCCACCTGGTATTACTATGACAGCATTCTCCCATCTTTCAGAGAGCTGGCCCTCTTGTTT
GAGCACATACAACGGGCCCGCAATGGGGGCTTACGGCCCCTGGAGGTGAGGCGGGCGCCAGTAC
CTGATGGAGCGCCCTCTCGCTACCCGCCCGTTGCCACCTGCCACCGGCCAGGCAGTGCCTCCTT
CTGCCCTGGGGAAAGCAGCCGGATGGGCATCAAAGACCGAATCCGCATAAGCAGCTCCCAGAAG
CGGACAGGCCCCTCCAAGCAGCATCTGGCCCCTCCGCCGATTCCCACCTCGCCAAGCAGTGAGC
AGGTGGGCGAGGCATCCAGCCCCAGCAAGGTGCAGAAAAGCTGGAGCTTCAATGACCGCACCCG
CTTCCGGGCCTCTCTAAGACTCAAGCCTCGCTGCTCTGCTGAGGAGGGCCCCTCAGAGGAAGTG
GCCGAGGAGAAGAGCTACCAGTGTGAGCTTACGGTGGATGATGTCATGCCTGCTGTGAAGACGG
TCATCCGTTCTGTCAGGATTCTGAAGTTCCTGGTAGCCAAAAGGAAATTCAAGGAGACGCTGCG
GCCATATGATGTGAAGGATGTCATTGAGCAGTATTCAGCAGGACACCTGGACATGCTGGGACGA
ATCAAGAGCCTGCAAGCCCGGGTGGACCAAATTGTGGGTCGGGGCCCTGGGGACCGCAAGACTC
GGGAGAAGGGCGATAAGGGTCCTTCAGACACGGAGGCAGTGGATGAAATCAGCATGATGGGGCG
CGTAGTCAAGGTGGAAAAGCAGGTGCAGTCCATCGAACATAAGTTGGATCTGCTGCTGGGCTTC
TACTCGCGCTGCCTCCGCTCTGGCACCTCGGCCAGCCTGGGCACTGTGCAAGTGCCGCTCTTCG
ACCCCGACATCACCTCGGACTACCACAGCCCTGTAGACCACGAGGACATCTCTGTCTCCGCACA
GACGCTCAGCATCTCTCGCTCAGTCAGCACCAACATGGAC

b. KCNQ4 Polypeptides

Among other things, the present disclosure provides polypeptides encoded by a KCNQ4 gene or gene product or characteristic portion thereof. In some embodiments, a KCNQ4 gene is a mammalian KCNQ4 gene. In some embodiments, a KCNQ4 gene is a murine KCNQ4 gene. In some embodiments, a KCNQ4 gene is a primate KCNQ4 gene. In some embodiments, a KCNQ4 gene is a human KCNQ4 gene.

In some embodiments, a polypeptide comprises a Kv7.4 protein or characteristic portion thereof. In some embodiments, a Kv7.4 protein or characteristic portion thereof is a mammalian Kv7.4 protein or characteristic portion thereof, e.g., primate Kv7.4 protein or characteristic portion thereof. In some embodiments, a Kv7.4 protein or characteristic portion thereof is a human Kv7.4 protein or characteristic portion thereof.

In some embodiments, a polypeptide provided herein comprises post-translational modifications. In some embodiments, a Kv7.4 protein or characteristic portion thereof provided herein comprises post-translational modifications. In some embodiments, post-translational modifications can comprise but is not limited to glycosylation (e.g., N-linked glycosylation, 0-linked glycosylation), phosphorylation, acetylation, amidation, hydroxylation, methylation, ubiquitylation, sulfation, and/or a combination thereof.

By way of non-limiting example, a KCNQ4 polypeptide in accordance with the present disclosure may be or comprise one or more of the following:

KCNQ4 EXEMPLARY HUMAN KCNQ4 POLYPEPTIDE ISOFORM
ASEQUENCE
(SEQ ID NO: 11)
MAEAPPRRLGLGPPPGDAPRAELVALTAVQSEQGEAGGGGSPRRLGLLGSPLPPGAPLPGPGSG
SGSACGQRSSAAHKRYRRLQNWVYNVLERPRGWAFVYHVFIFLLVFSCLVLSVLSTIQEHQELA
NECLLILEFVMIVVFGLEYIVRVWSAGCCCRYRGWQGRFRFARKPFCVIDFIVFVASVAVIAAG
TQGNIFATSALRSMRFLQILRMVRMDRRGGTWKLLGSWYAHSKELITAWYIGFLVLIFASFLV
YLAEKDANSDFSSYADSLWWGTITLTTIGYGDKTPHTWLGRVLAAGFALLGISFFALPAGILGS
GFALKVQEQHRQKHFEKRRMPAANLIQAAWRLYSTDMSRAYLTATWYYYDSILPSFRELALLFE
HVQRARNGGLRPLEVRRAPVPDGAPSRYPPVATCHRPGSTSFCPGESSRMGIKDRIRMGSSQRR
TGPSKQHLAPPTMPTSPSSEQVGEATSPTKVQKSWSFNDRTRFRASLRLKPRTSAEDAPSEEVA
EEKSYQCELTVDDIMPAVKTVIRSIRILKFLVAKRKFKETLRPYDVKDVIEQYSAGHLDMLGRI
KSLQTRVDQIVGRGPGDRKAREKGDKGPSDAEWDEISMMGRWKVEKQVQSIEHKLDLLLGFY
SRCLRSGTSASLGAVQVPLFDPDITSDYHSPVDHEDISVSAQTLSISRSVSTNMD
EXEMPLARY HUMAN KCNQ4 POLYPEPTIDE ISOFORM B
SEQUENCE
(SEQ ID NO: 12)
MAEAPPRRLGLGPPPGDAPRAELVALTAVQSEQGEAGGGGSPRRLGLLGSPLPPGAPLPGPGSG
SGSACGQRSSAAHKRYRRLQNWVYNVLERPRGWAFVYHVFIFLLVFSCLVLSVLSTIQEHQELA
NECLLILEFVMIVVFGLEYIVRVWSAGCCCRYRGWQGRFRFARKPFCVIDFIVFVASVAVIAAG
TQGNIFATSALRSMRFLQILRMVRMDRRGGTWKLLGSVVYAHSKELITAWYIGFLVLIFASFLV
YLAEKDANSDFSSYADSLWWGTITLTTIGYGDKTPHTWLGRVLAAGFALLGISFFALPAGILGS
GFALKVQEQHRQKHFEKRRMPAANLIQAAWRLYSTDMSRAYLTATWYYYDSILPSFSSRMGIKD
RIRMGSSQRRTGPSKQHLAPPTMPTSPSSEQVGEATSPTKVQKSWSFNDRTRFRASLRLKPRTS
AEDAPSEEVAEEKSYQCELTVDDIMPAVKTVIRSIRILKFLVAKRKFKETLRPYDVKDVIEQYS
AGHLDMLGRIKSLQTRVDQIVGRGPGDRKAREKGDKGPSDAEVVDEISMMGRVVKVEKQVQSIE
HKLDLLLGFYSRCLRSGTSASLGAVQVPLFDPDITSDYHSPVDHEDISVSAQTLSISRSVSTNM
D
EXEMPLARY HUMAN KCNQ4 POLYPEPTIDE ISOFORM XI
SEQUENCE
(SEQ ID NO: 13)
MPAANLIQAAWRLYSTDMSRAYLTATWYYYDSILPSFRELALLFEHVQRARNGGLRPLEVRRAP
VPDGAPSRYPPVATCHRPGSTSFCPGESSRMGIKDRIRMGSSQRRTGPSKQHLAPPTMPTSPSS
EQVGEATSPTKVQKSWSFNDRTRFRASLRLKPRTSAEDAPSEEVAEEKSYQCELTVDDIMPAVK
TVIRSIRILKFLVAKRKFKETLRPYDVKDVIEQYSAGHLDMLGRIKSLQTRVDQIVGRGPGDRK
AREKGDKGPSDAEVVDEISMMGRVVKVEKQVQSIEHKLDLLLGFYSRCLRSGTSASLGAVQVPL
FDPDITSDYHSPVDHEDISVSAQTLSISRSVSTNMD
EXEMPLARY MOUSE KCNQ4 POLYPEPTIDE MEMBER 4 SEQUENCE
(SEQ ID NO: 92)
MAEAPPRRLGLGPPPGDAPRAELVALTAVQSEQGEAGGGGSPRRLGLLGSPLPPGAPLPGPGSG
SGSACGGQRSSAAQKRYRRLQNWVYNVLERPRGWAFVYHVFIFLLVFSCLVLSVLSTIQEHQEL
ANECLLILEFVMIVVFGLEYIIRVWSAGCCCRYRGWQGRFRFARKPFCVIDFIVFVASVAVIAA
GTQGNIFATSALRSMRFLQILRMVRMDRRGGTWKLLGSVVYAHSKELITAWYIGFLVLIFASFL
VYLAEKDANSDFSSYADSLWWGTITLTTIGYGDKTPHTWLGRVLAAGFALLGISFFALPAGILG
SGFALKVQEQHRQKHFEKRRMPAANLIQAAWRLYSTDTSRAYLTATWYYYDSILPSFRELALLF
EHIQRARNGGLRPLEVRRAPVPDGAPSRYPPVATCHRPGSASFCPGESSRMGIKDRIRISSSQK
RTGPSKQHLAPPPIPTSPSSEQVGEASSPSKVQKSWSFNDRTRFRASLRLKPRCSAEEGPSEEV
AEEKSYQCELTVDDVMPAVKTVIRSVRILKFLVAKRKFKETLRPYDVKDVIEQYSAGHLDMLGR
IKSLQARVDQIVGRGPGDRKTREKGDKGPSDTEAVDEISMMGRWKVEKQVQSIEHKLDLLLGF
YSRCLRSGTSASLGTVQVPLFDPDITSDYHSPVDHEDISVSAQTLSISRSVSTNMD

c. Constructs

Among other things, the present disclosure provides that some polynucleotides as described herein are polynucleotide constructs. Polynucleotide constructs according to the present disclosure include all those known in the art, including cosmids, plasmids (e.g., naked or contained in liposomes) and viral constructs (e.g., lentiviral, retroviral, adenoviral, and adeno-associated viral constructs) that incorporate a polynucleotide comprising a KCNQ4 gene or characteristic portion thereof. Those of skill in the art will be capable of selecting suitable constructs, as well as cells, for making any of the nucleic acids described herein. In some embodiments, a construct is a plasmid (i.e., a circular DNA molecule that can autonomously replicate inside a cell). In some embodiments, a construct can be a cosmid (e.g., pWE or sCos series).

Constructs provided herein can be of different sizes. In some embodiments, a construct is a plasmid and can include a total length of up to about 1 kb, up to about 2 kb, up to about 3 kb, up to about 4 kb, up to about 5 kb, up to about 6 kb, up to about 7 kb, up to about 8 kb, up to about 9 kb, up to about 10 kb, up to about 11 kb, up to about 12 kb, up to about 13 kb, up to about 14 kb, or up to about 15 kb. In some embodiments, a construct is a plasmid and can have a total length in a range of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 1 kb to about 9 kb, about 1 kb to about 10 kb, about 1 kb to about 11 kb, about 1 kb to about 12 kb, about 1 kb to about 13 kb, about 1 kb to about 14 kb, or about 1 kb to about 15 kb.

In some embodiments, a construct is a viral construct and can have a total number of nucleotides of up to 10 kb. In some embodiments, a viral construct can have a total number of nucleotides in the range of about 1 kb to about 2 kb, 1 kb to about 3 kb, about 1 kb to about 4 kb, about 1 kb to about 5 kb, about 1 kb to about 6 kb, about 1 kb to about 7 kb, about 1 kb to about 8 kb, about 1 kb to about 9 kb, about 1 kb to about 10 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 2 kb to about 9 kb, about 2 kb to about 10 kb, about 3 kb to about 4 kb, about 3 kb to about 5 kb, about 3 kb to about 6 kb, about 3 kb to about 7 kb, about 3 kb to about 8 kb, about 3 kb to about 9 kb, about 3 kb to about 10 kb, about 4 kb to about 5 kb, about 4 kb to about 6 kb, about 4 kb to about 7 kb, about 4 kb to about 8 kb, about 4 kb to about 9 kb, about 4 kb to about 10 kb, about 5 kb to about 6 kb, about 5 kb to about 7 kb, about 5 kb to about 8 kb, about 5 kb to about 9 kb, about 5 kb to about 10 kb, about 6 kb to about 7 kb, about 6 kb to about 8 kb, about 6 kb to about 9 kb, about 6 kb to about 10 kb, about 7 kb to about 8 kb, about 7 kb to about 9 kb, about 7 kb to about 10 kb, about 8 kb to about 9 kb, about 8 kb to about 10 kb, or about 9 kb to about 10 kb.

In some embodiments, a construct is an adeno-associated virus (AAV) construct and can have a total number of nucleotides of up to 5 kb in a single construct. In some embodiments, an AAV construct can have a total number of nucleotides in the range of about 1 kb to about 2 kb, 1 kb to about 3 kb, about 1 kb to about 4 kb, about 1 kb to about 5 kb, about 2 kb to about 3 kb, about 2 kb to about 4 kb, about 2 kb to about 5 kb, about 3 kb to about 4 kb, about 3 kb to about 5 kb, about 4 kb to about 5 kb.

In some embodiments, a construct is a lentivirus construct and can have a total number of nucleotides of up to 8 kb. In some examples, a lentivirus construct can have a total number of nucleotides of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 3 kb to about 4 kb, about 3 kb to about 5 kb, about 3 kb to about 6 kb, about 3 kb to about 7 kb, about 3 kb to about 8 kb, about 4 kb to about 5 kb, about 4 kb to about 6 kb, about 4 kb to about 7 kb, about 4 kb to about 8 kb, about 5 kb to about 6 kb, about 5 kb to about 7 kb, about 5 kb to about 8 kb, about 6 kb to about 8 kb, about 6 kb to about 7 kb, or about 7 kb to about 8 kb

In some embodiments, a construct is an adenovirus construct and can have a total number of nucleotides of up to 8 kb. In some embodiments, an adenovirus construct can have a total number of nucleotides in the range of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 3 kb to about 4 kb, about 3 kb to about 5 kb, about 3 kb to about 6 kb, about 3 kb to about 7 kb, about 3 kb to about 8 kb, about 4 kb to about 5 kb, about 4 kb to about 6 kb, about 4 kb to about 7 kb, about 4 kb to about 8 kb, about 5 kb to about 6 kb, about 5 kb to about 7 kb, about 5 kb to about 8 kb, about 6 kb to about 7 kb, about 6 kb to about 8 kb, or about 7 kb to about 8 kb.

Any of the constructs described herein can further include a control sequence, e.g., a control sequence selected from the group of a transcription initiation sequence, a transcription termination sequence, a promoter sequence, an enhancer sequence, an RNA splicing sequence, a polyadenylation (polyA) sequence, a Kozak consensus sequence, and/or additional untranslated regions which may house pre- or post-transcriptional regulatory and/or control elements. In some embodiments, a promoter can be a native promoter, a constitutive promoter, an inducible promoter, and/or a tissue-specific promoter. Non-limiting examples of control sequences are described herein. The foregoing methods for producing recombinant constructs are not meant to be limiting, and other suitable methods will be apparent to the skilled artisan.

d. Viral Constructs

The present disclosure provides technologies (e.g., compositions, methods, etc.) that are or comprise viral constructs. In some embodiments, a viral construct is an adenovirus. In some embodiments, a viral construct is an adeno-associated virus (AAV). In some embodiments, a viral construct may also be based on an alphavirus. Alphaviruses include Sindbis (and VEEV) virus, Aura virus, Babanki virus, Barmah Forest virus, Bebaru virus, Cabassou virus, Chikungunya virus, Eastern equine encephalitis virus, Everglades virus, Fort Morgan virus, Getah virus, Highlands J virus, Kyzylagach virus, Mayaro virus, Me Tri virus, Middelburg virus, Mosso das Pedras virus, Mucambo virus, Ndumu virus, O′nyong-nyong virus, Pixuna virus, Rio Negro virus, Ross River virus, Salmon pancreas disease virus, Semliki Forest virus, Southern elephant seal virus, Tonate virus, Trocara virus, Una virus, Venezuelan equine encephalitis virus, Western equine encephalitis virus, and Whataroa virus. Generally, genomes of such viruses encode nonstructural (e.g., replicon) and structural proteins (e.g., capsid and envelope) that can be translated in cytoplasm of a host cell. Ross River virus, Sindbis virus, Semliki Forest virus (SFV), and Venezuelan equine encephalitis virus (VEEV) have all been used to develop viral transfer constructs for transgene delivery. Pseudotyped viruses may be formed by combining alphaviral envelope glycoproteins and retroviral capsids. Examples of alphaviral constructs can be found in U.S. Publication Nos. 20150050243, 20090305344, and 20060177819; constructs and methods of their making are incorporated herein by reference in their entireties.

i. AAV constructs

Recombinant AAV constructs (“rAAVs”; see, e.g., Asokan et al., Mol. Ther. 20: 699-7080, 2012, which is herein incorporated by reference in its entirety) of the disclosure are typically comprised of (i) a transgene or a portion thereof and a regulatory sequence, and (ii) 5′ and 3′ AAV inverted terminal repeats (ITRs). It is this recombinant AAV construct which is packaged into a capsid protein and delivered to a selected target cell. In some embodiments, the transgene is a nucleic acid sequence (e.g., inhibitory nucleic acid sequence), heterologous to the construct sequences, which encodes a polypeptide, protein, functional RNA molecule (e.g., miRNA, miRNA inhibitor) or other gene product, of interest. A nucleic acid coding sequence is operatively linked to regulatory components in a manner which permits transgene transcription, translation, and/or expression in a cell of a target tissue. In some embodiments, a recombinant AAV construct is packaged into a capsid to form an rAAV particle and delivered to a selected target cell (e.g., an outer hair cell).

In some embodiments, and rAAV construct also comprises conventional control elements that are operably linked to a transgene in a manner which permits its transcription, translation and/or expression in a cell transfected with a plasmid construct or infected with a virus produced by the disclosure.

AAV constructs as described in the present disclosure may include one or more additional elements as described herein (e.g., regulatory elements e.g., one or more of a promoter, a polyA sequence, and an IRES).

Methods for obtaining viral constructs are known in the art. For example, to produce AAV constructs, methods typically involve culturing a host cell which comprises a nucleic acid sequence encoding an AAV capsid protein or fragment thereof; a functional rep gene; a recombinant AAV construct comprising an AAV inverted terminal repeats (ITRs) and a transgene; and/or sufficient helper functions to permit packaging of the recombinant AAV construct into AAV capsid proteins.

In some embodiments, components to be cultured in a host cell to package an AAV construct in an AAV capsid may be provided to the host cell in trans. Alternatively, one or more components (e.g., recombinant AAV construct, rep sequences, cap sequences, and/or helper functions) may be provided by a stable host cell that has been engineered to contain one or more such components using methods known to those of skill in the art. In some embodiments, such a stable host cell contains such component(s) under control of an inducible promoter. In some embodiments, such component(s) may be under control of a constitutive promoter. In some embodiments, a selected stable host cell may contain selected component(s) under control of a constitutive promoter and other selected component(s) under control of one or more inducible promoters. For example, a stable host cell may be generated that is derived from HEK293 cells (which contain E1 helper functions under the control of a constitutive promoter), but that contain rep and/or cap proteins under control of inducible promoters. Other stable host cells may be generated by one of skill in the art using routine methods.

Recombinant AAV construct, rep sequences, cap sequences, and helper functions required for producing an AAV of the disclosure may be delivered to a packaging host cell using any appropriate genetic element (e.g., construct). A selected genetic element may be delivered by any suitable method known in the art, e.g., to those with skill in nucleic acid manipulation and include genetic engineering, recombinant engineering, and synthetic techniques (see, e.g., Sambrook et al., Molecular Cloning: A Laboratory Manual, Cold Spring Harbor Press, Cold Spring Harbor, N.Y., which is incorporated in its entirety herein by reference). Similarly, methods of generating AAV virions are well known and any suitable method can be used with the present disclosure (see, e.g., K. Fisher et al., J. Virol., 70:520-532 (1993) and U.S. Pat. No. 5,478,745, each of which is incorporated in its entirety herein by reference).

In some embodiments, recombinant AAVs may be produced using a triple transfection method (e.g., as described in U.S. Pat. No. 6,001,650, which is incorporated in its entirety herein by reference). In some embodiments, recombinant AAVs are produced by transfecting a host cell with a recombinant AAV construct (comprising a transgene) to be packaged into AAV particles, an AAV helper function construct, and an accessory function construct. An AAV helper function construct encodes “AAV helper function” sequences (i.e., rep and cap), which function in trans for productive AAV replication and encapsidation. In some embodiments, an AAV helper function construct supports efficient AAV construct production without generating any detectable wild-type AAV virions (i.e., AAV virions containing functional rep and cap genes). Non-limiting examples of constructs suitable for use with the present disclosure include pHLP19 (see, e.g., U.S. Pat. No. 6,001,650, which is incorporated in its entirety herein by reference) and pRep6cap6 construct (see, e.g., U.S. Pat. No. 6,156,303, which is incorporated in its entirety herein by reference). An accessory function construct encodes nucleotide sequences for non-AAV derived viral and/or cellular functions upon which AAV is dependent for replication (i.e., “accessory functions”). Accessory functions may include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any known helper viruses such as adenovirus, herpesvirus (other than herpes simplex virus type-1), and vaccinia virus.

Additional methods for generating and isolating AAV viral constructs suitable for delivery to a subject are described in, e.g., U.S. Pat. Nos. 7,790,449; 7,282,199; WO 2003/042397; WO 2005/033321, WO 2006/110689; and U.S. Pat. No. 7,588,772, each of which is incorporated in its entirety herein by reference. In one system, a producer cell line is transiently transfected with a construct that encodes a transgene flanked by ITRs and a construct(s) that encodes rep and cap. In another system, a packaging cell line that stably supplies rep and cap is transiently transfected with a construct encoding a transgene flanked by ITRs. In each of these systems, in some embodiments, AAV virions are produced in response to infection with helper adenovirus or herpesvirus, and AAVs are separated from contaminating virus. In some embodiments, systems do not require infection with helper virus to recover the AAV. As will be understood to those of skill in the art, in some embodiments, a helper function is or comprises at least one of e.g., adenovirus E1, E2a, VA, and E4 or herpesvirus UL5, UL8, UL52, and UL29, and herpesvirus polymerase. In some such embodiments, a helper function is supplied, in trans, by or to a given system. In some such embodiments, helper functions can be supplied by transient transfection of cells with constructs that encode helper functions. In some embodiments, cells can be engineered to stably contain genes encoding at least one helper function. In some such embodiments, where cells are stably engineered to contain genes encoding helper function(s) helper function expression can be controlled at a transcriptional or posttranscriptional level.

AAV serotypes

As described herein, in some embodiments, a viral construct of the present disclosure is an adeno-associated virus (AAV) construct. AAV systems are generally well known in the art (see, e.g., Kelleher and Vos, Biotechniques, 17(6):1110-17 (1994); Cotten et al., P.N.A.S. U.S.A., 89(13):6094-98 (1992); Curiel, Nat Immun, 13(2-3):141-64 (1994); Muzyczka, Curr Top Microbiol Immunol, 158:97-129 (1992); and Asokan A, et al., Mol. Ther., 20(4):699-708 (2012), each of which is incorporated in its entirety herein by reference). Methods for generating and using AAV constructs are described, for example, in U.S. Pat. Nos. 5,139,941 and 4,797,368, each of which is incorporated in its entirety herein by reference. Several AAV serotypes have been characterized, including AAV1, AAV2, AAV3 (e.g., AAV3B), AAV4, AAV5, AAV6, AAV7, AAV8, AAV9, AAV10, and AAV11, as well as variants thereof. In some embodiments, an AAV construct is an AAV2/6, AAV2/8 or AAV2/9 construct (e.g., AAV6, AAV8 or AAV9 serotype having AAV2 ITR). Other AAV constructs are described in, e.g., Sharma et al., Brain Res Bull. 2010 Feb. 15; 81(2-3): 273, which is incorporated in its entirety herein by reference. Generally, any AAV serotype may be used to deliver a transgene described herein. However, serotypes are known to have different tropisms, e.g., they preferentially infect different tissues. In some embodiments, an AAV construct is a self-complementary AAV construct.

iii. Capsids

In some embodiments, one or more recombinant AAV constructs of the present disclosure is packaged into a capsid of AAV2, 3, 4, 5, 6, 7, 8, 9, 10, rh8, rh10, rh39, rh43, AAV2.7m8, AAV8BP2, or Anc80 serotype or one or more hybrids thereof. In some embodiments, a capsid is from an ancestral serotype. For example, in some embodiments, a capsid is an Anc80 capsid (e.g., an Anc80L65 capsid). In some embodiments, a capsid comprises a polypeptide represented by SEQ ID NO: 14. In some embodiments, a capsid comprises a polypeptide with at least 85%, 90%, 95%, 98% or 99% sequence identity to a polypeptide of SEQ ID NO: 14.

Any combination of ITRs and capsids may be used in recombinant AAV constructs of the present disclosure, for example, wild-type or variant AAV2 ITRs and Anc80 capsid, wild-type or variant AAV2 ITRs and AAV6 capsid, etc. In some embodiments of the present disclosure, an rAAV particle is wholly comprised of AAV2 components (i.e., capsid and ITRs are AAV2 serotype). In some embodiments of the present disclosure an rAAV particle is an rAAV2/Anc80 particle which comprises an Anc80 capsid (e.g., comprising a polypeptide of SEQ ID NO: 14) that encapsidates a nucleic acid construct with wild-type AAV2 ITRs (e.g., any of SEQ ID NOs: 15, 16, 17, 18, 19, 20 and/or 21) flanking a portion of a construct comprising all or a characteristic portion of a KCNQ4 encoding sequence (e.g., SEQ ID NOs:1-10). In some embodiments, an ITR is at least 85%, 90%, 95%, 98% or 99% identical to an ITR of SEQ ID NOs: 15, 16, 17, 18, 19, 20, or 21.

Anc80L65 Capsid
(SEQ ID NO: 14)
MAADGYLPDWLEDNLSEGIREWWDLKPGAPKPKANQQKQDDGRGLVLPGY
KYLGPFNGLDKGEPVNAADAAALEHDKAYDQQLKAGDNPYLRYNHADAEF
QERLQEDTSFGGNLGRAVFQAKKRVLEPLGLVEEGAKTAPGKKRPVEQSP
QEPDSSSGIGKKGQQPARKRLNFGQTGDSESVPDPQPLGEPPAAPSGVGS
NTMAAGGGAPMADNNEGADGVGNASGNWHCDSTWLGDRVITTSTRTWALP
TYNNHLYKQISSQSGGSTNDNTYFGYSTPWGYFDFNRFHCHFSPRDWQRL
INNNWGFRPKKLNFKLFNIQVKEVTTNDGTTTIANNLTSTVQVFTDSEYQ
LPYVLGSAHQGCLPPFPADVFMIPQYGYLTLNNGSQAVGRSSFYCLEYFP
SQMLRTGNNFQFSYTFEDVPFHSSYAHSQSLDRLMNPLIDQYLYYLSRTQ
TTSGTAGNRTLQFSQAGPSSMANQAKNWLPGPCYRQQRVSKTTNQNNNSN
FAWTGATKYHLNGRDSLVNPGPAMATHKDDEDKFFPMSGVLIFGKQGAGN
SNVDLDNVMITNEEEIKTTNPVATEEYGTVATNLQSANTAPATGTVNSQG
ALPGMVWQDRDVYLQGPIWAKIPHTDGHEHPSPLMGGFGLKHPPPQILIK
NTPVPANPPTTFSPAKFASFITQYSTGQVSVEIEWELQKENSKRWNPEIQ
YTSNYNKSTNVDFAVDTNGVYSEPRPIGTRYLTRNL

iv. Inverted Terminal Repeat Sequences (ITRs)

AAV sequences of a construct typically comprise the cis-acting 5′ and 3′ inverted terminal repeat sequences (See, e.g., B. J. Carter, in “Handbook of Parvoviruses,” ed., P. Tijsser, CRC Press, pp. 155 168 (1990), which is incorporated in its entirety herein by reference). In some embodiments, ITR sequences are about 145 nt in length. For example, wild type AAV2 ITRs are generally about 145 nt in length. Preferably, substantially the entire sequences encoding ITRs are used in a given molecule, although some degree of minor modification of these sequences is permissible. Ability to modify ITR sequences is within the skill of the art. (See, e.g., texts such as Sambrook et al. “Molecular Cloning. A Laboratory Manual,” 2d ed., Cold Spring Harbor Laboratory, New York (1989); and K. Fisher et al., J Virol., 70:520 532 (1996), each of which is incorporated in its entirety herein by reference). An example of such a molecule employed in the present disclosure is a “cis-acting” construct comprising a sequence encoding a gene product (e.g., a KCNQ4 gene product) or inhibitory nucleic acid thereof (e.g., an miRNA), in which such a sequence and its associated regulatory elements are flanked by 5′ or “left” and 3′ or “right” AAV ITR sequences. 5′ and left designations refer to a position of an ITR sequence relative to an entire construct, read left to right, in a sense direction. For example, in some embodiments, a 5′ or left ITR is an ITR that is closest to a promoter (as opposed to a polyadenylation sequence) for a given construct, when a construct is depicted in a sense orientation, linearly. 3′ and right designations refer to a position of an ITR sequence relative to an entire construct, read left to right, in a sense direction. For example, in some embodiments, a 3′ or right ITR is an ITR that is closest to a polyadenylation sequence (as opposed to a promoter sequence) for a given construct, when a construct is depicted in a sense orientation, linearly. ITRs as provided herein are depicted in 5′ to 3′ order in accordance with a sense strand. Accordingly, one of skill in the art will appreciate that a 5′ or “left” orientation ITR can also be depicted as a 3′ or “right” ITR when converting from sense to antisense direction. Further, it is well within the ability of one of skill in the art to transform a given sense ITR sequence (e.g., a 5′/left AAV ITR) into an antisense sequence (e.g., 3′/right ITR sequence). Accordingly, based upon known AAV ITRs one of skill in the art would understand, in looking at sequences disclosed herein, whether an ITR was in a sense or antisense orientation and whether it would go on a “left” or “right” side of a construct, whether or not it is explicitly labeled as such. One of ordinary skill in the art would understand how to modify a given ITR sequence for use as either a 5′/left or 3′/right ITR, or an antisense version thereof.

AAV ITR sequences may be obtained from any known AAV, including presently identified mammalian AAV types. In some embodiments, an ITR is or comprises 145 nucleotides. In some embodiments an ITR is a wild-type AAV2 ITR, e.g., the 5′ ITR of SEQ ID NO: 15 and the 3′ ITR of SEQ ID NO: 16. In some embodiments an ITR is derived from a wild-type AAV2 ITR and includes one or more modifications, e.g., truncations, deletions, substitutions or insertions as is known in the art. In some embodiments, an ITR comprises fewer than 145 nucleotides (e.g., SEQ ID NO:s 19 or 20), e.g., 119, 127, 130, 134 or 141 nucleotides (see, e.g., SEQ ID Nos: 17, 18, 19, and 20. For example, in some embodiments, an ITR comprises 110, 111, 112, 113, 114, 115, 116, 117, 118, 119, 120, 121, 122, 123,124, 125, 126, 127, 128, 129, 130, 131, 132, 133, 134, 135, 136, 137, 138, 139, 140, 141, 142, 143 144, or 145 nucleotides.

A non-limiting example of a 5′/left AAV ITR sequence is SEQ ID NO: 17. A non-limiting example of a 3′/right AAV ITR sequence is SEQ ID NO: 18. In some embodiments, constructs and/or constructs of the present disclosure comprise a 5′/left AAV ITR and/or a 3′/right AAV ITR. In some embodiments, a 5′/left AAV ITR sequence is SEQ ID NO: 16. In some embodiments, a 3′/right AAV ITR sequence is SEQ ID NO: 16. In some embodiments, a 5′/left AAV ITR sequence is SEQ ID NO: 15 and a 3′/right AAV ITR sequence is SEQ ID NO: 16. In some embodiments, an ITR is at least 85%, 90%, 95%, 98% or 99% identical to the ITR represented by SEQ ID NOs: 15, 16, 17, 18, or 19. In some embodiments, 5′/left and a 3′/right AAV ITRs (e.g., SEQ ID NOs: 15 and 16) flank a portion of a transgene and/or construct comprising all or a portion of a KCNQ4 gene product (e.g., SEQ ID NOs: 1-10).

In some embodiments, an ITR sequence is at least 85%, 90%, 95%, 98% or 99% identical to any ITR sequence disclosed herein. By way of non-limiting example, ITR sequences may be or comprise the following:

5′/Left AAV ITR
(SEQ ID NO: 15)
TTGGCCACTCCCTCTCTGCGCGCTCGCTCGCTCACTGAGGCCGCCCGGGCAAAGCCCGGGCGTC
GGGCGACCTTTGGTCGCCCGGCCTCAGTGAGCGAGCGAGCGCGCAGAGAGGGAGTGGCCAACTC
CATCACTAGGGGTTCCT
3′/Right AAV ITR
(SEQ ID NO: 16)
AGGAACCCCTAGTGATGGAGTTGGCCACTCCCTCTCTGCGCGCTCGCTCGCTCACTGAGGCCGG
GCGACCAAAGGTCGCCCGACGCCCGGGCTTTGCCCGGGCGGCCTCAGTGAGCGAGCGAGCGCGC
AGAGAGGGAGTGGCCAA
5′/Left AAV ITR
(SEQ ID NO: 17)
CCTGCAGGCAGCTGCGCGCTCGCTCGCTCACTGAGGCCGCCCGGGCGTCGGGCGACCTTTGGTC
GCCCGGCCTCAGTGAGCGAGCGAGCGCGCAGAGAGGGAGTGGCCAACTCCATCACTAGGGGTT
3′/Right AAV ITR
(SEQ ID NO: 18)
AGGAACCCCTAGTGATGGAGTTGGCCACTCCCTCTCTGCGCGCTCGCTCGCTCACTGAGGCCGG
GCGACCAAAGGTCGCCCGACGCCCGGGCTTTGCCCGGGCGGCCTCAGTGAGCGAGCGAGCGCGC
AGCTGCCTGCAGG
AAV ITR
(SEQ ID NO: 19)
CTGCGCGCTCGCTCGCTCACTGAGGCCGCCCGGGCGTCGGGCGACCTTTGGTCGCCCGGCCTCA
GTGAGCGAGCGAGCGCGCAGAGAGGGAGTGGCCAACTCCATCACTAGGGGTTCCT
AAV ITR
(SEQ ID NO: 20)
AGGAACCCCTAGTGATGGAGTTGGCCACTCCCTCTCTGCGCGCTCGCTCGCTCACTGAGGCCGG
GCGACCAAAGGTCGCCCGACGCCCGGGCTTTGCCCGGGCGGCCTCAGTGAGCGAGCGAGCGCGC
AG
AAV ITR
(SEQ ID NO: 21)
TTGGCCACTCCCTCTCTGCGCGCTCGCTCGCTCACTGAGGCCGCCCGGGCAAAGCCCGGGCGTC
GGGCGACCTTTGGTCGCCCGGCCTCAGTGAGCGAGCGAGCGCGCAGAGAGGGAGTGGCCAACTC
CATCACTAGGGGTTCCT

v. Promoters

Non-limiting examples of promoters are described herein. Additional examples of promoters are known in the art.

In some embodiments, a construct (e.g., an rAAV construct) comprises a promoter. The term “promoter” refers to a DNA sequence recognized by enzymes/proteins that can promote and/or initiate transcription of an operably linked gene (e.g., an KCNQ4 gene or inhibitory nucleic acid thereof). In some embodiments, a construct encoding a KCNQ4 gene product (e.g., a human Kv7.4 protein, etc.) or inhibitory nucleic acid thereof (e.g., an miRNA, etc.) can include a promoter and/or an enhancer. For example, a promoter typically refers to, e.g., a nucleotide sequence to which an RNA polymerase and/or any associated factor binds and from which it can initiate transcription. Thus, in some embodiments, a construct (e.g., an rAAV construct) comprises a promoter operably linked to one of the non-limiting example promoters described herein.

In some embodiments, a promoter is an inducible promoter, a constitutive promoter, a mammalian cell promoter, a viral promoter, a chimeric promoter, an engineered promoter, a tissue-specific promoter, or any other type of promoter known in the art. In some embodiments, a promoter is a RNA polymerase II promoter, such as a mammalian RNA polymerase II promoter. In some embodiments, a promoter is a RNA polymerase III promoter, including, but not limited to, a HI promoter, a human U6 promoter, a mouse U6 promoter, or a swine U6 promoter. A promoter will generally be one that is able to promote transcription in an inner ear cell. In some embodiments, a promoter is a cochlea-specific promoter or a cochlea-oriented promoter. In some embodiments, a promoter is a hair cell specific promoter, or a supporting cell specific promoter.

A variety of promoters are known in the art, which can be used herein. Non-limiting examples of promoters that can be used herein include: human EF1α, human cytomegalovirus (CMV) (U.S. Pat. No. 5,168,062, which is incorporated in its entirety herein by reference), human ubiquitin C (UBC), mouse phosphoglycerate kinase 1, polyoma adenovirus, simian virus 40 (SV40), β-globin, β-actin, α-fetoprotein, γ-globin, β-interferon, γ-glutamyl transferase, mouse mammary tumor virus (MMTV), Rous sarcoma virus, rat insulin, glyceraldehyde-3-phosphate dehydrogenase, metallothionein II (MT II), amylase, cathepsin, MI muscarinic receptor, retroviral LTR (e.g., human T-cell leukemia virus HTLV), AAV ITR, interleukin-2, collagenase, platelet-derived growth factor, adenovirus 5 E2, stromelysin, murine MX gene, glucose regulated proteins (GRP78 and GRP94), α-2-macroglobulin, vimentin, MHC class I gene H-2κ b, HSP70, proliferin, tumor necrosis factor, thyroid stimulating hormone a gene, immunoglobulin light chain, T-cell receptor, HLA DQa and DQ, interleukin-2 receptor, MEW class II, MEW class II HLA-DRa, muscle creatine kinase, prealbumin (transthyretin), elastase I, albumin gene, c-fos, c-HA-ras, neural cell adhesion molecule (NCAM), H2B (TH2B) histone, rat growth hormone, human serum amyloid (SAA), troponin I (TN I), duchenne muscular dystrophy, human immunodeficiency virus, and Gibbon Ape Leukemia Virus (GALV) promoters. Additional examples of promoters are known in the art. See, e.g., Lodish, Molecular Cell Biology, Freeman and Company, New York 2007, each of which is incorporated in its entirety herein by reference. In some embodiments, a promoter is the CMV immediate early promoter.

In some embodiments, a promoter is an inducible promoter, a mammalian cell promoter, a viral promoter, a chimeric promoter, an engineered promoter, a constitutive promoter, a tissue-specific promoter, or any other type of promoter known in the art.

In some embodiments, a promoter is a RNA polymerase II promoter, such as a mammalian RNA polymerase II promoter.

In some embodiments, a promoter is a RNA polymerase III promoter (e.g., an H1 promoter, a U6 promoter (e.g., a human U6 promoter, a mouse U6 promoter, a swine U6 promoter, etc.).

In some embodiments, a promoter of the present disclosure will generally be one that is able to function (i.e., transcribe), in cochlear cells such as hair cells, e.g., IHCs, e.g., OHCs.

In some embodiments, a promoter is a cochlea-specific promoter or a cochlea-oriented promoter.

A variety of promoters is known in the art, any of which can be used herein. Non-limiting examples of promoters that can be used herein include: human elongation factor 1α-subunit (EF1a) (Liu et al. (2007) Exp. Mol. Med. 39(2): 170-175; Accession No. J04617.1; Gill et al., Gene Ther. 8(20):1539-1546, 2001; Xu et al., Human Gene Ther. 12(5):563-573, 2001; Xu et al., Gene Ther. 8:1323-1332; Ikeda et al., Gene Ther. 9:932-938, 2002; Gilham et al., J. Gene Med. 12(2):129-136, 2010, each of which is incorporated in its entirety herein by reference), cytomegalovirus (Xu et al., Human Gene Ther. 12(5):563-573, 2001; Xu et al., Gene Ther. 8:1323-1332; Gray et al., Human Gene Ther. 22:1143-1153, 2011, each of which is incorporated in its entirety herein by reference), human immediate-early cytomegalovirus (CMV) (U.S. Pat. No. 5,168,062, Liu et al. (2007) Exp. Mol. Med. 39(2): 170-175; Accession No. X17403.1 or KY490085.1, each of which is incorporated in its entirety herein by reference), human ubiquitin C (UBC) (Gill et al., Gene Ther. 8(20):1539-1546, 2001; Qin et al., PLoS One 5(5):e10611, 2010, each of which is incorporated in its entirety herein by reference), mouse phosphoglycerate kinase 1, polyoma adenovirus, simian virus 40 (SV40), β-globin, β-actin, α-fetoprotein, γ-globin, β-interferon, γ-glutamyl transferase, mouse mammary tumor virus (MMTV), Rous sarcoma virus, rat insulin, glyceraldehyde-3-phosphate dehydrogenase, metallothionein II (MT II), amylase, cathepsin, MI muscarinic receptor, retroviral LTR (e.g., human T-cell leukemia virus HTLV, each of which is incorporated in its entirety herein by reference), AAV ITR, interleukin-2, collagenase, platelet-derived growth factor, adenovirus 5 E2, stromelysin, murine MX gene, glucose regulated proteins (GRP78 and GRP94), α-2-macroglobulin, vimentin, MHC class I gene H-2-κ b, HSP70, proliferin, tumor necrosis factor, thyroid stimulating hormone a gene, immunoglobulin light chain, T-cell receptor, HLA DQα and DQβ, interleukin-2 receptor, MHC class II, MHC class II HLA-DRα, muscle creatine kinase, prealbumin (transthyretin), elastase I, albumin gene, c-fos, c-HA-ras, neural cell adhesion molecule (NCAM), H2B (TH2B) histone, rat growth hormone, human serum amyloid (SAA), troponin I (TN I), duchenne muscular dystrophy, human immunodeficiency virus, Gibbon Ape Leukemia Virus (GALV) promoters, promoter of HNRPA2B1-CBX1 (UCOE) (Powell and Gray (2015) Discov. Med. 19(102): 49-57; Antoniou et al., Human Gene Ther. 24(4):363-374, 2013), β-glucuronidase (GUSB) (Husain et al., Gene Ther. 16:927-932, 2009), chicken β-actin (CBA) (Liu et al. (2007) Exp. Mol. Med. 39(2): 170-175; Stone et al. (2005) Mol. Ther. 11(6): 843-848; Klein et al., Exp. Neurol. 176(1):66-74, 2002; Ohlfest et al., Blood 105:2691-2698, 2005; Gray et al., Human Gene Ther. 22:1143-1153, 2011, each of which is incorporated in its entirety herein by reference), a human β-actin promoter (HBA) (Accession No. Y00474.1), murine myosin VIIA (musMyo7) (Boeda et al. (2001) Hum. Mol. Genet. 10(15): 1581-1589; Accession No. AF384559.1, each of which is incorporated in its entirety herein by reference), human myosin VIIA (hsMyo7) (Boeda et al. (2001) Hum. Mol. Genet. 10(15): 1581-1589; Accession No. NG_009086.1, each of which is incorporated in its entirety herein by reference), murine poly(ADP-ribose) polymerase 2 (musPARP2) (Ame et al. (2001) J. Biol. Chem. 276(14): 11092-11099; Accession No. AF191547.1, each of which is incorporated in its entirety herein by reference), human poly(ADP-ribose) polymerase 2 (hsPARP2) (Ame et al. (2001) J. Biol. Chem. 276(14): 11092-11099; Accession No. X16612.1 or AF479321.1, each of which is incorporated in its entirety herein by reference), acetylcholine receptor epsilon-subunit (AChε) (Duclert et al. (1993) PNAS 90(7): 3043-3047; Accession No. S58221.1 or CR933736.12, each of which is incorporated in its entirety herein by reference), Rous sarcoma virus (RSV) (Liu et al. (2007) Exp. Mol. Med. 39(2): 170-175; Accession No. M77786.1, each of which is incorporated in its entirety herein by reference), (GFAP) (Liu et al. (2007) Exp. Mol. Med. 39(2): 170-175; Stone et al. (2005) Mol. Ther. 11(6): 843-848; Accession No. NG_008401.1 or M67446.1, each of which is incorporated in its entirety herein by reference), hAAT (Van Linthout et al., Human Gene Ther. 13(7):829-840, 2002; Cunningham et al., Mol. Ther. 16(6):1081-1088, 2008, each of which is incorporated in its entirety herein by reference), and a CBA hybrid (CBh) (Gray et al. (2011) Human Gene Therapy 22: 1143-1153; Accession No. KF926476.1 or KC152483.1, each of which is incorporated in its entirety herein by reference). Additional examples of promoters are known in the art. See, e.g., Lodish, Molecular Cell Biology, Freeman and Company, New York 2007.

In some embodiments, a promoter is a CMV immediate early promoter.

In some embodiments, a promoter is a CAG promoter or a CAG/CBA promoter.

In some embodiments, a promoter is an smCBA promoter.

In some embodiments, a construct or construct of the present disclosure comprises a CAG promoter. In some embodiments, a CAG promoter comprises, in order from 5′ to 3′, nucleotide sequences of SEQ ID NOs: 22, 23, and 24. In some such embodiments, a CAG promoter comprises a CMV early enhancer element (e.g., SEQ ID NO: 22 or SEQ ID NO: 298 or SEQ ID NO: 299), a chicken beta actin (CBA) gene sequence (e.g., SEQ ID NO: 23), and a chimeric intron/3′ splice sequence from a rabbit beta globin gene (e.g., SEQ ID NO: 24). In some embodiments, a promoter is at least 85%, 90%, 95%, 98% or 99% identical to CAG promoter represented by SEQ ID NOs: 22, 23, 24, 300, or 301.

The term “constitutive” promoter refers to a nucleotide sequence that, when operably linked with a nucleic acid encoding a protein (e.g., a KCNQ4 protein) or an inhibitory nucleic acid (e.g., as described herein), causes RNA to be transcribed from the nucleic acid in a cell under most or all physiological conditions.

Examples of constitutive promoters include, without limitation, a retroviral Rous sarcoma virus (RSV) LTR promoter, a cytomegalovirus (CMV) promoter (see, e.g., Boshart et al. Cell 41:521-530, 1985, which is incorporated in its entirety herein by reference), an SV40 promoter, a dihydrofolate reductase promoter, a beta-actin promoter, a phosphoglycerol kinase (PGK) promoter, and an EF1-alpha promoter (Invitrogen).

In some embodiments, inducible promoters allow regulation of gene expression and can be regulated by exogenously supplied compounds, environmental factors such as temperature, or presence of a specific physiological state, e.g., acute phase, a particular functional or biological state of a cell, e.g., a particular differentiation state of a cell, or in replicating cells only. Inducible promoters and inducible systems are available from a variety of commercial sources, including, without limitation, Invitrogen, Clontech, and Ariad. Additional examples of inducible promoters are known in the art.

Examples of inducible promoters regulated by exogenously supplied compounds include a zinc-inducible sheep metallothionine (MT) promoter, a dexamethasone (Dex)—inducible mouse mammary tumor virus (MMTV) promoter, a T7 polymerase promoter system (WO 98/10088, which is incorporated in its entirety herein by reference); an ecdysone insect promoter (No et al. Proc. Natl. Acad. Sci. U.S.A. 93:3346-3351, 1996, which is incorporated in its entirety herein by reference), a tetracycline-repressible system (Gossen et al. Proc. Natl. Acad. Sci. U.S.A. 89:5547-5551, 1992, which is incorporated in its entirety herein by reference), a tetracycline-inducible system (Gossen et al. Science 268:1766-1769, 1995, see also Harvey et al. Curr. Opin. Chem. Biol. 2:512-518, 1998, each of which is incorporated in its entirety herein by reference), an RU486-inducible system (Wang et al. Nat. Biotech. 15:239-243, 1997; and Wang et al. Gene Ther. 4:432-441, 1997, each of which is incorporated in its entirety herein by reference), and a rapamycin-inducible system (Magari et al. J. Clin. Invest. 100:2865-2872, 1997, which is incorporated in its entirety herein by reference).

In some embodiments, regulatory sequences impart tissue-specific gene expression capabilities. In some cases, tissue-specific regulatory sequences bind tissue-specific transcription factors that induce transcription in a tissue-specific manner.

The term “tissue-specific” promoter refers to a promoter that is active only in certain specific cell types and/or tissues (e.g., transcription of a specific gene occurs only within cells expressing transcription regulatory and/or control proteins that bind to the tissue-specific promoter).

In some embodiments, a tissue-specific promoter is a cochlea-specific promoter. In some embodiments, the tissue-specific promoter is a cochlear hair cell-specific promoter. Non-limiting examples of cochlear hair cell-specific promoters include but are not limited to an ATOH1 promoter, a POU4F3 promoter, an LHX3 promoter, a MYO7A promoter, a MYO6 promoter, an α9ACHR promoter, and aa α10ACHR promoter. In some embodiments, a promoter is a cochlear hair cell-specific promoter such as a PRESTIN promoter or an ONCOMOD promoter. See, e.g., Zheng et al., Nature 405:149-155, 2000; Tian et al. Dev. Dyn. 23 1: 199-203, 2004; and Ryan et al., Adv. Otorhinolaryngol. 66: 99-115, 2009, each of which is incorporated in their entirety herein by reference.

In some embodiments, a tissue-specific promoter is an ear cell specific promoter. In some embodiments, a tissue-specific promoter is an inner ear cell specific promoter. Non-limiting examples of inner ear non-sensory cell-specific promoters include but are not limited to: GJB2, GJB6, SLC26A4, TECTA, DFNA5, COCH, NDP, SYN1, GFAP, PLP, TAK1, or SOX21. In some embodiments, a cochlear non-sensory cell specific promoter may be an inner ear supporting cell specific promoter. Non-limiting examples of inner ear supporting cell specific promoters include but are not limited to: SOX2, FGFR3, PROX1, GLAST1, LGR5, HES1, HES5, NOTCH1, JAG1, CDKN1A, CDKN1B, SOX10, P75, CD44, HEY2, LFNG, or S100b.

In some embodiments, provided AAV constructs comprise a promoter sequence selected from a CAG, a CBA, a CMV, or a CB7 promoter. In some embodiments of any of the therapeutic compositions described herein, the first or sole AAV construct further includes at least one promoter sequence selected from Cochlea and/or inner ear specific promoters.

In some embodiments, a promoter sequence is at least 85%, 90%, 95%, 98% or 99% identical to any promoter sequence disclosed herein. By way of non-limiting example, promoter sequences provided in accordance with the present disclosure may be or comprise the following:

CMV Enhancer
(SEQ ID NO: 22)
GACATTGATTATTGACTAGTTATTAATAGTAATCAATTACGGGGTCATTAGTTCATAGCCCATA
TATGGAGTTCCGCGTTACATAACTTACGGTAAATGGCCCGCCTGGCTGACCGCCCAACGACCCC
CGCCCATTGACGTCAATAATGACGTATGTTCCCATAGTAACGCCAATAGGGACTTTCCATTGAC
GTCAATGGGTGGACTATTTACGGTAAACTGCCCACTTGGCAGTACATCAAGTGTATCATATGCC
AAGTACGCCCCCTATTGACGTCAATGACGGTAAATGGCCCGCCTGGCATTATGCCCAGTACATG
ACCTTATGGGACTTTCCTACTTGGCAGTACATCTACGTATTAGTCATCGCTATTACCATGG
CBA Gene Sequence
(SEQ ID NO: 23)
GTCGAGGTGAGCCCCACGTTCTGCTTCACTCTCCCCATCTCCCCCCCCTCCCCACCCCCAATTT
TGTATTTATTTATTTTTTAATTATTTTGTGCAGCGATGGGGGCGGGGGGGGGGGGGGGGCGCGC
GCCAGGCGGGGCGGGGCGGGGCGAGGGGCGGGGCGGGGCGAGGCGGAGAGGTGCGGCGGCAGCC
AATCAGAGCGGCGCGCTCCGAAAGTTTCCTTTTATGGCGAGGCGGCGGCGGCGGCGGCCCTATA
AAAAGCGAAGCGCGCGGCGGGCG
CBA promoter
(SEQ ID NO: 297)
GTCGAGGTGAGCCCCACGTTCTGCTTCACTCTCCCCATCTCCCCCCCCTCCCCACCCCCAATTT
TGTATTTATTTATTTTTTAATTATTTTGTGCAGCGATGGGGGCGGGGGGGGGGGGGGCGCGCGC
CAGGCGGGGCGGGGCGGGGCGAGGGGCGGGGCGGGGCGAGGCGGAGAGGTGCGGCGGCAGCCAA
TCAGAGCGGCGCGCTCCGAAAGTTTCCTTTTATGGCGAGGCGGCGGCGGCGGCGGCCCTATAAA
AAGCGAAGCGCGCGGCGGGCG
CMV/CBA enhancer/promoter
(SEQ ID NO: 298)
GACATTGATTATTGACTAGTTATTAATAGTAATCAATTACGGGGTCATTAGTTCATAGCCCATA
TATGGAGTTCCGCGTTACATAACTTACGGTAAATGGCCCGCCTGGCTGACCGCCCAACGACCCC
CGCCCATTGACGTCAATAATGACGTATGTTCCCATAGTAACGCCAATAGGGACTTTCCATTGAC
GTCAATGGGTGGACTATTTACGGTAAACTGCCCACTTGGCAGTACATCAAGTGTATCATATGCC
AAGTACGCCCCCTATTGACGTCAATGACGGTAAATGGCCCGCCTGGCATTATGCCCAGTACATG
ACCTTATGGGACTTTCCTACTTGGCAGTACATCTACGTATTAGTCATCGCTATTACCATGGGTC
GAGGTGAGCCCCACGTTCTGCTTCACTCTCCCCATCTCCCCCCCCTCCCCACCCCCAATTTTGT
ATTTATTTATTTTTTAATTATTTTGTGCAGCGATGGGGGCGGGGGGGGGGGGGGGGCGCGCGCC
AGGCGGGGCGGGGCGGGGCGAGGGGCGGGGCGGGGCGAGGCGGAGAGGTGCGGCGGCAGCCAAT
CAGAGCGGCGCGCTCCGAAAGTTTCCTTTTATGGCGAGGCGGCGGCGGCGGCGGCCCTATAAAA
AGCGAAGCGCGCGGCGGGCG
CMV/CBA enhancer/promoter
(SEQ ID NO: 299)
GACATTGATTATTGACTAGTTATTAATAGTAATCAATTACGGGGTCATTAGTTCATAGCCCATA
TATGGAGTTCCGCGTTACATAACTTACGGTAAATGGCCCGCCTGGCTGACCGCCCAACGACCCC
CGCCCATTGACGTCAATAATGACGTATGTTCCCATAGTAACGCCAATAGGGACTTTCCATTGAC
GTCAATGGGTGGACTATTTACGGTAAACTGCCCACTTGGCAGTACATCAAGTGTATCATATGCC
AAGTACGCCCCCTATTGACGTCAATGACGGTAAATGGCCCGCCTGGCATTATGCCCAGTACATG
ACCTTATGGGACTTTCCTACTTGGCAGTACATCTACGTATTAGTCATCGCTATTACCATGGGTC
GAGGTGAGCCCCACGTTCTGCTTCACTCTCCCCATCTCCCCCCCCTCCCCACCCCCAATTTTGT
ATTTATTTATTTTTTAATTATTTTGTGCAGCGATGGGGGCGGGGGGGGGGGGGGCGCGCGCCAG
GCGGGGCGGGGCGGGGCGAGGGGCGGGGCGGGGCGAGGCGGAGAGGTGCGGCGGCAGCCAATCA
GAGCGGCGCGCTCCGAAAGTTTCCTTTTATGGCGAGGCGGCGGCGGCGGCGGCCCTATAAAAAG
CGAAGCGCGCGGCGGGCG
CAG enhancer/promoter
(SEQ ID NO: 300)
GACATTGATTATTGACTAGTTATTAATAGTAATCAATTACGGGGTCATTAGTTCATAGCCCATA
TATGGAGTTCCGCGTTACATAACTTACGGTAAATGGCCCGCCTGGCTGACCGCCCAACGACCCC
CGCCCATTGACGTCAATAATGACGTATGTTCCCATAGTAACGCCAATAGGGACTTTCCATTGAC
GTCAATGGGTGGACTATTTACGGTAAACTGCCCACTTGGCAGTACATCAAGTGTATCATATGCC
AAGTACGCCCCCTATTGACGTCAATGACGGTAAATGGCCCGCCTGGCATTATGCCCAGTACATG
ACCTTATGGGACTTTCCTACTTGGCAGTACATCTACGTATTAGTCATCGCTATTACCATGGGTC
GAGGTGAGCCCCACGTTCTGCTTCACTCTCCCCATCTCCCCCCCCTCCCCACCCCCAATTTTGT
ATTTATTTATTTTTTAATTATTTTGTGCAGCGATGGGGGCGGGGGGGGGGGGGGGGCGCGCGCC
AGGCGGGGCGGGGCGGGGCGAGGGGCGGGGCGGGGCGAGGCGGAGAGGTGCGGCGGCAGCCAAT
CAGAGCGGCGCGCTCCGAAAGTTTCCTTTTATGGCGAGGCGGCGGCGGCGGCGGCCCTATAAAA
AGCGAAGCGCGCGGCGGGCGGGAGTCGCTGCGTTGCCTTCGCCCCGTGCCCCGCTCCGCGCCGC
CTCGCGCCGCCCGCCCCGGCTCTGACTGACCGCGTTACTCCCACAGGTGAGCGGGCGGGACGGC
CCTTCTCCTCCGGGCTGTAATTAGCGCTTGGTTTAATGACGGCTCGTTTCTTTTCTGTGGCTGC
GTGAAAGCCTTAAAGGGCTCCGGGAGGGCCCTTTGTGCGGGGGGGAGCGGCTCGGGGGGTGCGT
GCGTGTGTGTGTGCGTGGGGAGCGCCGCGTGCGGCCCGCGCTGCCCGGCGGCTGTGAGCGCTGC
GGGCGCGGCGCGGGGCTTTGTGCGCTCCGCGTGTGCGCGAGGGGAGCGCGGCCGGGGGCGGTGC
CCCGCGGTGCGGGGGGGCTGCGAGGGGAACAAAGGCTGCGTGCGGGGTGTGTGCGTGGGGGGGT
GAGCAGGGGGTGTGGGCGCGGCGGTCGGGCTGTAACCCCCCCCTGCACCCCCCTCCCCGAGTTG
CTGAGCACGGCCCGGCTTCGGGTGCGGGGCTCCGTGCGGGGCGTGGCGCGGGGCTCGCCGTGCC
GGGCGGGGGGTGGCGGCAGGTGGGGGTGCCGGGCGGGGCGGGGCCGCCTCGGGCCGGGGAGGGC
TCGGGGGAGGGGCGCGGCGGCCCCCGGAGCGCCGGCGGCTGTCGAGGCGCGGCGAGCCGCAGCC
ATTGCCTTTTATGGTAATCGTGCGAGAGGGCGCAGGGACTTCCTTTGTCCCAAATCTGTGCGGA
GCCGAAATCTGGGAGGCGCCGCCGCACCCCCTCTAGCGGGCGCGGGGCGAAGCGGTGCGGCGCC
GGCAGGAAGGAAATGGGCGGGGAGGGCCTTCGTGCGTCGCCGCGCCGCCGTCCCCTTCTCCCTC
TCCAGCCTCGGGGCTGTCCGCGGGGGGACGGCTGCCTTCGGGGGGGACGGGGCAGGGCGGGGTT
CGGCTTCTGGCGTGTGACCGGCGGCTCTAGAGCCTCTGCTAACCATGTTCATGCCTTCTTCTTT
TTCCTAGAG
CAG enhancer/promoter
(SEQ ID NO: 301)
GAGATTGATTATTGAGTAGTTATTAATAGTAATCAATTACGGGGTCATTAGTTCATAGCCCATA
TATGGAGTTCCGCGTTACATAACTTACGGTAAATGGCCCGCCTGGCTGACCGCCCAACGACCCC
CGCCCATTGAGGTCAATAATGAGGTATGTTCCCATAGTAACGCCAATAGGGAGTTTCCATTGAG
GTCAATGGGTGGACTATTTACGGTAAACTGCCCACTTGGCAGTACATCAAGTGTATCATATGCC
AAGTACGCCCCCTATTGACGTCAATGACGGTAAATGGCCCGCCTGGCATTATGCCCAGTACATG
ACCTTATGGGACTTTCCTACTTGGCAGTACATCTACGTATTAGTCATCGCTATTACCATGGGTC
GAGGTGAGCCCCACGTTCTGCTTCACTCTCCCCATCTCCCCCCCCTCCCCACCCCCAATTTTGT
ATTTATTTATTTTTTAATTATTTTGTGCAGCGATGGGGGCGGGGGGGGGGGGGGCGCGCGCCAG
GCGGGGCGGGGCGGGGCGAGGGGCGGGGCGGGGCGAGGCGGAGAGGTGCGGCGGCAGCCAATCA
GAGCGGCGCGCTCCGAAAGTTTCCTTTTATGGCGAGGCGGCGGCGGCGGCGGCCCTATAAAAAG
CGAAGCGCGCGGCGGGCGGGAGTCGCTGCGTTGCCTTCGCCCCGTGCCCCGCTCCGCGCCGCCT
CGCGCCGCCCGCCCCGGCTCTGACTGACCGCGTTACTCCCACAGGTGAGCGGGCGGGACGGCCC
TTCTCCTCCGGGCTGTAATTAGCGCTTGGTTTAATGACGGCTCGTTTCTTTTCTGTGGCTGCGT
GAAAGCCTTAAAGGGCTCCGGGAGGGCCCTTTGTGCGGGGGGGAGCGGCTCGGGGGGTGCGTGC
GTGTGTGTGTGCGTGGGGAGCGCCGCGTGCGGCCCGCGCTGCCCGGCGGCTGTGAGCGCTGCGG
GCGCGGCGCGGGGCTTTGTGCGCTCCGCGTGTGCGCGAGGGGAGCGCGGCCGGGGGCGGTGCCC
CGCGGTGCGGGGGGGCTGCGAGGGGAACAAAGGCTGCGTGCGGGGTGTGTGCGTGGGGGGGTGA
GCAGGGGGTGTGGGCGCGGCGGTCGGGCTGTAACCCCCCCCTGCACCCCCCTCCCCGAGTTGCT
GAGCACGGCCCGGCTTCGGGTGCGGGGCTCCGTGCGGGGCGTGGCGCGGGGCTCGCCGTGCCGG
GCGGGGGGTGGCGGCAGGTGGGGGTGCCGGGCGGGGCGGGGCCGCCTCGGGCCGGGGAGGGCTC
GGGGGAGGGGCGCGGCGGCCCCCGGAGCGCCGGCGGCTGTCGAGGCGCGGCGAGCCGCAGCCAT
TGCCTTTTATGGTAATCGTGCGAGAGGGCGCAGGGACTTCCTTTGTCCCAAATCTGTGCGGAGC
CGAAATCTGGGAGGCGCCGCCGCACCCCCTCTAGCGGGCGCGGGGCGAAGCGGTGCGGCGCCGG
CAGGAAGGAAATGGGCGGGGAGGGCCTTCGTGCGTCGCCGCGCCGCCGTCCCCTTCTCCCTCTC
CAGCCTCGGGGCTGTCCGCGGGGGGACGGCTGCCTTCGGGGGGGACGGGGCAGGGCGGGGTTCG
GCTTCTGGCGTGTGACCGGCGGCTCTAGAGCCTCTGCTAACCATGTTCATGCCTTCTTCTTTTT
CCTAGAG
Chimeric Intron
(SEQ ID NO: 24)
GGAGTCGCTGCGTTGCCTTCGCCCCGTGCCCCGCTCCGCGCCGCCTCGCGCCGCCCGCCCCGGC
TCTGACTGACCGCGTTACTCCCACAGGTGAGCGGGCGGGACGGCCCTTCTCCTCCGGGCTGTAA
TTAGCGCTTGGTTTAATGACGGCTCGTTTCTTTTCTGTGGCTGCGTGAAAGCCTTAAAGGGCTC
CGGGAGGGCCCTTTGTGCGGGGGGGAGCGGCTCGGGGGGTGCGTGCGTGTGTGTGTGCGTGGGG
AGCGCCGCGTGCGGCCCGCGCTGCCCGGCGGCTGTGAGCGCTGCGGGCGCGGCGCGGGGCTTTG
TGCGCTCCGCGTGTGCGCGAGGGGAGCGCGGCCGGGGGCGGTGCCCCGCGGTGCGGGGGGGCTG
CGAGGGGAACAAAGGCTGCGTGCGGGGTGTGTGCGTGGGGGGGTGAGCAGGGGGTGTGGGCGCG
GCGGTCGGGCTGTAACCCCCCCCTGCACCCCCCTCCCCGAGTTGCTGAGCACGGCCCGGCTTCG
GGTGCGGGGCTCCGTGCGGGGCGTGGCGCGGGGCTCGCCGTGCCGGGCGGGGGGTGGCGGCAGG
TGGGGGTGCCGGGCGGGGCGGGGCCGCCTCGGGCCGGGGAGGGCTCGGGGGAGGGGCGCGGCGG
CCCCCGGAGCGCCGGCGGCTGTCGAGGCGCGGCGAGCCGCAGCCATTGCCTTTTATGGTAATCG
TGCGAGAGGGCGCAGGGACTTCCTTTGTCCCAAATCTGTGCGGAGCCGAAATCTGGGAGGCGCC
GCCGCACCCCCTCTAGCGGGCGCGGGGCGAAGCGGTGCGGCGCCGGCAGGAAGGAAATGGGCGG
GGAGGGCCTTCGTGCGTCGCCGCGCCGCCGTCCCCTTCTCCCTCTCCAGCCTCGGGGCTGTCCG
CGGGGGGACGGCTGCCTTCGGGGGGGACGGGGCAGGGCGGGGTTCGGCTTCTGGCGTGTGACCG
GCGGCTCTAGAGCCTCTGCTAACCATGTTCATGCCTTCTTCTTTTTCCTACAG

In certain embodiments, a promoter is an endogenous human ATOH1 enhancer-promoter as set forth in SEQ ID NO: 302. In some embodiments, an enhancer-promoter sequence is at least 85%, 90%, 95%, 98% or 99% identical to enhancer-promoter sequence represented by SEQ ID NO: 302.

Human ATOH1 enhancer-promoter
(SEQ ID NO: 302)
CTATGGAGTTTGCATAACAAACGTTTGGCAGCTCGCTCTCTTACACTCCA
TTAACAAGCTGTAACATATAGCTGCAGGTTGCTATAATCTCATTAATATT
TTGGAAACTTGAATATTGAGTATTTCTGAGTGCTCATTCCCCATATGCCA
GCCACTTCTGCCATGCTGACTGGTTCCTTTCTCTCCATTATTAGCAATTA
GCTTCTTACCTTCCAAAGTCAGATCCAAGGTATCCAAGATACTAGCAAAG
GAATCAACTATGTGTGCAAGTTAAGCATGCTTAATATCACCCAAACAAAC
AAAGAGGCAGCATTTCTTAAAGTAATGAAGATAGATAAATCGGGTTAGTC
CTTTGCGACACTGCTGGTGCTTTCTAGAGTTTTATATATTTTAAGCAGCT
TGCTTTATATTCTGTCTTTGCCTCCCACCCCACCAGCACTTTTATTTGTG
GAGGGTTTTGGCTCGCCACACTTTGGGAAACTTATTTGATTTCACGGAGA
GCTGAAGGAAGATCATTTTTGGCAACAGACAAGTTTAAACACGATTTCTA
TGGGACATTGCTAACTGGGGCCCCTAAGGAGAAAGGGGAAACTGAGCGGA
GAATGGGTTAAATCCTTGGAAGCAGGGGAGAGGCAGGGGAGGAGAGAAGT
CGGAGGAGTATAAAGAAAAGGACAGGAACCAAGAAGCGTGGGGGTGGTTT
GCCGTAATGTGAGTGTTTCTTAATTAGAGAACGGTTGACAATAGAGGGTC
TGGCAGAGGCTCCTGGCCGCGGTGCGGAGCGTCTGGAGCGGAGCACGCGC
TGTCAGCTGGTGAGCGCACTCTCCTTTCAGGCAGCTCCCCGGGGAGCTGT
GCGGCCACATTTAACACCATCATCACCCCTCCCCGGCCTCCTCAACCTCG
GCCTCCTCCTCGTCGACAGCCTTCCTTGGCCCCCACCAGCAGAGCTCACA
GTAGCGAGCGTCTCTCGCCGTCTCCCGCACTCGGCCGGGGCCTCTCTCCT
CCCCCAGCTGCGCAGCGGGAGCCGCCACTGCCCACTGCACCTCCCAGCAA
CCAGCCCAGCACGCAAAGAAGCTGCGCAAAGTTAAAGCCAAGCAATGCCA
AGGGGAGGGGAAGCTGGAGGCGGGCTTTGAGTGGCTTCTGGGCGCCTGGC
GGGTCCAGAATCGCCCAGAGCCGCCCGCGGTCGTGCACATCTGACCCGAG
TCAGCTTGGGCACCAGCCGAGAGCCGGCTCCGCACCGCTCCCGCACCCCA
GCCGCCGGGGTGGTGACACACACCGGAGTCGAATTACAGGCCTGCAATTA
ACATATGAATCTGAGGAATTTAAAAGAAGGAAAAAAAAAAAAAAACCTGA
GCAGGCTTGGGAGTCCTCTGCACACAAGAACTTTTCTCGGGGTGTAAAAA
CTCTTTGATTGGCTGCTCGCACGCGCCTGCCCGCGCCCTCCATTGGCTGA
GAAGACACGCGACCGGCGCGAGGAGGGGGTTGGGAGAGGAGCGGGGGGAG
ACTGAGTGGCGCGTGCCGCTTTTTAAAGGGGCGCAGCGCCTTCAGCAACC
GGAGAAGCATAGTTGCACGCGACCTGGTGTGTGATCTCCGAGTGGGTGGG
GGAGGGTCGAGGAGGGAAAAAAAAATAAGACGTTGCAGAAGAGACCCGGA
AAGGGCCTTTTTTTTGGTTGAGCTGGTGTCCCAGTGCTGCCTCCGATCCT
GAGCCTCCGAGCCTTTGCAGTGCAA

In certain embodiments, a promoter is an endogenous human SLC26A4 immediate promoter as set forth in SEQ ID NO: 303 or 304. In certain embodiments, a promoter is an endogenous human SLC26A4 enhancer-promoter as set forth in SEQ ID NO: 305, 306, or 307. In some embodiments, an enhancer-promoter sequence is at least 85%, 90%, 95%, 98% or 99% identical to a promoter or enhancer-promoter sequence represented by SEQ ID NO: 303, 304, 305, 306, or 307. In certain embodiments, a promoter is a human SLC26A4 endogenous enhancer-promoter sequence comprised within SEQ ID NO: 305, 306, or 307.

Human SLC26A4 immediate promoter
(SEQ ID NO: 303)
CTGCCTTCTGAGAGCGCTATAAAGGCAGCGGAAGGGTAGTCCGCGGGGCATTCCGGGCGG
Human SLC26A4 immediate promoter
(SEQ ID NO: 304)
CTCTAGGCGGGCTCTGCTCTTCTTTAAGGAGTCCCACAGGGCCTGGCCCGCCCCTGACCT
Human SLC26A4 enhancer-promoter
(SEQ ID NO: 305)
TAAAGAGTTGTGAGTTGTGTAGGTGAGTTGCCATGGAGCTACAAATATGAGTTGATATTCTGAA
ATCCTAGACAGCCATCTCCAAGGTTAAGAAAAATCCTTATGCACTCACTTGCAAAGATATCCAC
AGCATGCTCTTAATGGAGAAAAACAAAGCCTTAGATCAAATATGTAAAGTAATTTTTAGTTTTT
TGAAAAGGTATGTTTGGGCTATAGATAAATCTGTTCAAAAAACATGAGAGAAGATAATAATGGT
TGAAAGGAGACACAGTGCTTGCCCTCAAGAAGTTTTTGTCTAGTGAGGGAGAGAGAACTTGTAT
GTAAATAAAATTGTGTTACTAAGGTAGATAGTGAGAAGTAACTTAAGAGAGGATCAGATAAGGT
ATTAAGAGAATACAGAAAAGGGTCTGGATTAATTCTGAACAGCATCAAAGAATGTTCTTGCAAG
AGATAGTGTTTTCACCAGATCTTGAAGGTATGGATGAGGGTATACAGAGTGAGTATATTCAGAT
TCTACTTTAAAACAAATACTTTCCTCTGTTGTAGTGGAGTTGAGCTATACATCCAACAATAATG
AAAAAATACACGCATATATACATATATGGAGAGAGATACATATTTTAGTAGATGTAGCAATTGA
TTAATAAATGTACAGTTTAAGTCGCATGCAAAACCTTGGAGTGATAGCAAACTTCATTGTAGGA
TGTTTAGCAGCATCTCTGGTCTCTACTCACTAGATCCCAATAGCATCTCCCTAGGTGTGACAAC
CAAAAATGTCTCCAGGCATTGACCTCTGGAGGCAAAAAAAGCCCTTTATTAAGAACCAGTGGTA
TACATAAGTAAAACATACACAAGAGATTCCTCCCCTCTTCTCTGTATGTGAATAAAAATTGCAA
AGTTCATGACCTGGATTTTCCTTTTAGGTTTCTTCTTTAGTGGTTCTTAACTTCATTGGGTGAA
GTAAGCCTTTGAAGATCTGTTGAAAGCTGTTGACTCATTCACTTCTCAGGAAAACGCACATGCT
GACTACCATTTCAGAGAATTTGCATCAGGGTTCTCTGGGGAGGAGTTCTGAGTTCTGTTTCCAG
GAGCTCGTAGAATTGTCATGGTCTGCATATGCAAGGCAGGTGGATTACGGAAGGTTGATGTACA
GAGGTCTGTATTTTGGAGCCTCTTCTGTATTTACTTCAGAACACTAACAATCAGGCGAGAATGT
TCTGGTTTATCAAACCCTTCCTTCTGCCTTTCATCTTAACCATGCATTAGTTTTAACAAAGTTC
ATCCCAACAGAAGACAAAACACTGATGAGGTAGGATAGCTCCAGCTCCTCCTCCCTCTCTTCTA
GTCTTGATTTCCATGTAGTCCAGTTTATTCCTTCCCTGATTGTCCAGGAGAATGAGAAAAAGAA
AAAACAGAGTCTAGTGGGTAAGAAAGGGCCACCTGGACGGCTTGATTTGGATTGTGAAATAAAA
CACACACACATGCACACGTAGAATAAGTGGCTAAAATCTGAGTAAATCGTGAACTCTCTGTATC
CTCCACCCATTGAATACTCCTAAAAGACTTTCTAGAAATTCAAGGACTTATTAATATAGAAACC
TGGCCATTGTTCCTCTTCTCCTCCCCATGTGGTATGAGAGCACCTGTGGCAGGCTCCCAGAGAC
CACGGACCTCTTCCTCTAGGCGGGCTCTGCTCTTCTTTAAGGAGTCCCACAGGGCCTGGCCCGC
CCCTGACCTCGCAACCCTTGAGATTAGTAACGGGATGAGTGAGGATCCGGGTGGCCCCTGCGTG
GCAGCCAGTAAGAGTCTCAGCCTTCCCGGTTCGGGAAAGGGGAAGAATGCAGGAGGGGTAGGAT
TTCTTTCCTGATAGGATCGGTTGGGAAAGACCGCAGCCTGTGTGTGTCTTTCCCTTCGACCAAG
GTGTCTGTTGCTCCGTAAATAAAACGTCCCACTGCCTTCTGAGAGCGCTATAAAGGCAGCGGAA
GGGTAGTCCGCGGGGC
Human SLC26A4 enhancer-promoter
(SEQ ID NO: 306)
GGCTGCTCGGAAAACAGGACGAGGGGAGAGACTTGCTCAATAAGCTGAAAGTTCTGCCCCCGAG
AGGGCTGCGACAGCTGCTGGAATGTGCCTGCAGCGTCCGCCTCTTGGGGACCCGCGGAGCGCGC
CCTGACGGTTCCACGCCTGGCCCGGGGGTCTGCACCTCTCCTCCAGTGCGCACCTGGAGCTGCG
TCCCGGGTCAGGTGCGGGGAGGGAGGGAATCTCAGTGTCCCCTTCCAGCCTTGCAAGCGCCTTT
GGCCCCTGCCCCAGCCCCTCGGTTTGGGGGAGATTTCAGAACGCGGACAGCGCCCTGGCTGCGG
GCCATAGGGGACTGGGTGGAACTCGGGAAGCCCCCAGAGCAGGGGCTTACTCGCTTCAAGTTTG
GGGAACCCCGGGCAGCGGGTGCAGGCCACGAGACCCGAAGGTTCTCAGGTGCCCCCCTGCAGGC
TGGCCGTGCGCGCCGTGGGGCGCTTGTCGCGAGCGCCGAGGGCTGCAGGACGCGGACCAGACTC
GCGGTGCAGGGGGGCCTGGCTGCAGCTAACAGGTGATCCCGTTCTTTCTGTTCCTCGCTCTTCC
CCTCCGATCGTCCTCGCTTACCGCGTGTCCTCCCTCCTCGCTGTCCTCTGGCTCGCAGGTCATG
GCAGCGCCAGGCGGCAGGTCGGAGCCGCCGCAGCTCCCCGAGTACAGCTGCAGCTACATGGTGT
CGCGGCCGGTCTACAGCGAGCTCGCTTTCCAGCAACAGCACGAGCGGCGCCTGCAGGAGCGCAA
GACGCTGCGGGAGAGCCTGGCCAAGTGCTGCAGGTAGCGGCCGCGCGGGCCTGCGTAGAGAGAA
GCGGAGCGGGGCGTCCACGCCTTGGGGAGGGAAGGGCGTCCCCAGCGGGCGAGAGTGGGGTGCG
GGCGGCGGAGCCCCTGGGCGCCAGCTGCTTCTCCCAGAGGCCCGACTTTCGGTCTCCGGTCCTC
CACGCCGCCCTTCTGGTGGGAGGGTGGCTCCATCAGTCTCGGGCCCGAAATGAACTTACCTGGG
AAACTCGCCTTTGGGGAGAGTGGGTTCTAGGAGCCCCGTCTCTCTTTTTCCTCTCTGAAGGAAA
CTTGGAGTGCCTCTTGGGGTACAGTGGGTCCCTGTTGCCTTCTTGGGAGCTTGTTTAAATGAAA
TGAATAGGGAAACCCAGCTCTTGACCAGGAGGAGTCCTTGAAACACTCAAGCTAAGTAGGCGGG
CTACCATTCAGTTAGAGACCAGGATGCAAGCTAGAACCCAGGGGAGCGCGGGGTGTGCCAAGTA
CTTCATCAGCAGGCTGTGGGACCCCTGGGGAAAGCCACCCTCAGTCTCTAAACCCAAACATGCC
GTAACTAGATGTCACAAACATAAAGAAATTAGAGTTTCTAAAACCTTTCATTATAG
Human SLC26A4 enhancer-promoter
(SEQ ID NO: 307)
CGGAAGGTTGATGTACAGAGGTCTGTATTTTGGAGCCTCTTCTGTATTTACTTCAGAACACTAA
CAATCAGGCGAGAATGTTCTGGTTTATCAAACCCTTCCTTCTGCCTTTCATCTTAACCATGCAT
TAGTTTTAACAAAGTTCATCCCAACAGAAGACAAAACACTGATGAGGTAGGATAGCTCCAGCTC
CTCCTCCCTCTCTTCTAGTCTTGATTTCCATGTAGTCCAGTTTATTCCTTCCCTGATTGTCCAG
GAGAATGAGAAAAAGAAAAAACAGAGTCTAGTGGGTAAGAAAGGGCCACCTGGACGGCTTGATT
TGGATTGTGAAATAAAACACACACACATGCACACGTAGAATAAGTGGCTAAAATCTGAGTAAAT
CGTGAACTCTCTGTATCCTCCACCCATTGAATACTCCTAAAAGACTTTCTAGAAATTCAAGGAC
TTATTAATATAGAAACCTGGCCATTGTTCCTCTTCTCCTCCCCATGTGGTATGAGAGCACCTGT
GGCAGGCTCCCAGAGACCACGGACCTCTTCCTCTAGGCGGGCTCTGCTCTTCTTTAAGGAGTCC
CACAGGGCCTGGCCCGCCCCTGACCTCGCAACCCTTGAGATTAGTAACGGGATGAGTGAGGATC
CGGGTGGCCCCTGCGTGGCAGCCAGTAAGAGTCTCAGCCTTCCCGGTTCGGGAAAGGGGAAGAA
TGCAGGAGGGGTAGGATTTCTTTCCTGATAGGATCGGTTGGGAAAGACCGCAGCCTGTGTGTGT
CTTTCCCTTCGACCAAGGTGTCTGTTGCTCCGTAAATAAAACGTCCCACTGCCTTCTGAGAGCG
CTATAAAGGCAGCGGAAGGGTAGTCCGCGGGGCATTCCGGGCGGGGCGCGAGCAGAGACAGGTG
AGTT

In certain embodiments, a promoter is a human LGR5 enhancer-promoter as set forth in SEQ ID NO: 308. In some embodiments, an enhancer-promoter sequence is at least 85%, 90%, 95%, 98% or 99% identical to enhancer-promoter sequence represented by SEQ ID NO: 308. In some embodiments, a promoter is a human LGR5 endogenous enhancer-promoter sequence comprised within SEQ ID NO: 308.

Human LGR5 enhancer-promoter
(SEQ ID NO: 308)
AGGGCTATTTGTACCTCAACGAGGGCTTCTCTCCAAGAAAGCCCTGAATC
CTTTTCCTCCTTTTTCCTGCAGATTCACTATAGGACACTTTTTGAAGCAA
GAGCATGCATTTTCCCCCTGGCGCTCTGCAGCGGTTCTCAGAGCCCAGTG
TCACTCACATAGGTGGGACTGCTCTCAGTTCAGAGAGCGCTGGGACACTT
AAGATGAAAAGTCCCTGGAAGTTAGCAAACAGCCATCTGTCACTCTGGCA
TCGATTTAGTAAAAGTGAGTTCTAGGGTATTCTAAACGAGTTTTAAAAAA
CAAATGAGTGAGTTCGAGTTCCTCACCCCGCAAGAGATAGGAAGGCAGCA
GTGGAGTGCTCGCTCAGGAGCTGTATTTGTTTAGCGATTAGCCTAGAGCT
TTGATTTTAGGGCAAAAGCGAGCCAGACAGTGCGGCAGACGTAAGGATCA
AAAAGGCCACCTATCATTCGCCGGGGACGCCTGCCTCCTTACCCTGATAA
CGTAACTATTTCTCTGCATAGGATTTTAGTTTTTGTGTTTTTGTTTTGTT
TTATTCTGTTTAATCACTTCAAGTATCTCATCCATTATTTGAAGCGGGCT
CGGAGGAAACGTGCCGCATCCTCCAGTCCTTGTGCGTCTGTTTAGGTCTC
TCCGAAGCAGGTCCCTCTCGACTCTTAGATCTGGGTCTCCAGCACGCATG
AAGGGGTAAGGGTGGGGGGGTCCCCTATTCCGGCGCGCGGCGTTGAGCAC
TGAATCTTCCAGGCGGAGGCTCAGTGGGAGCGCCGAGAACTCGCCAGTAC
CGCGCGCTGCCTGCTGCCTGCTGCCTCCCAGCCCAGGACTTGGGAAAGGA
GGGAGGGGACAAGTGGAGGGAAAGTGGGGCCGGGCGGGGGGTGCCTGGGA
AGCCAGGCTGCGCTGACGTCACTGGGCGCGCAATTCGGGCTGGAGCGCTT
TAAAAAACGAGCGTGCAAGCAGAGATGCTGCTCCACACCGCTCAGGCCGC
GAGCAGCAGCAAGGCGCACCGCCACTGTCGCCGCTGCAGCCAGGGCTGCT
CCGAAGGCCGGCGTGGCGGCAACCGGCACCTCTGTCCCCGCCGCGCTTCT
CCTCGCCGCCCACGCCGTGGGGTCAGGAACGCGGCGTCTGGCGCTGCAGA
CGCCCGCTGAGTTGCAGAAGCCCACGGAGCGGCGCCCGGCGCGCCACGGC
CCGTAGCAGTCCGGTGCTGCTCTCCGCCCGCGTCCGGCTCGTGGCCCCCT
ACTTCGGGCACCGACCGGT

In certain embodiments, a promoter is a human SYN1 enhancer-promoter as set forth in SEQ ID NO: 309. In some embodiments, an enhancer-promoter sequence is at least 85%, 90%, 95%, 98% or 99% identical to enhancer-promoter sequence represented by SEQ ID NO: 309. In some embodiments, a promoter is a human SYN1 endogenous enhancer-promoter sequence comprised within SEQ ID NO: 309.

Human SYN1 enhancer-promoter
(SEQ ID NO: 309)
TGCGTATGAGTGCAAGTGGGTTTTAGGACCAGGATGAGGCGGGGTGGGGG
TGCCTACCTGACGACCGACCCCGACCCACTGGACAAGCACCCAACCCCCA
TTCCCCAAATTGCGCATCCCCTATCAGAGAGGGGGAGGGGAAACAGGATG
CGGCGAGGCGCGTGCGCACTGCCAGCTTCAGCACCGCGGACAGTGCCTTC
GCCCCCGCCTGGCGGCGCGCGCCACCGCCGCCTCAGCACTGAAGGCGCGC
TGACGTCACTCGCCGGTCCCCCGCAAACTCCCCTTCCCGGCCACCTTGGT
CGCGTCCGCGCCGCCGCCGGCCCAGCCGGACCGCACCACGCGAGGCGCGA
GATAGGGGGGCACGGGCGCGACCATCTGCGCTGCGGCGCCGGCGACTCAG
CGCTGCCTCAGTCTGCGGTGGGCAGCGGAGGAGTCGTGTCGTGCCTGAGA
GCGCAGTCGAGAA

In certain embodiments, a promoter is a human GFAP enhancer-promoter as set forth in SEQ ID NO: 310. In some embodiments, an enhancer-promoter sequence is at least 85%, 90%, 95%, 98% or 99% identical to enhancer-promoter sequence represented by SEQ ID NO: 310. In some embodiments, a promoter is a human GFAP endogenous enhancer-promoter sequence comprised within SEQ ID NO: 310.

Human GFAP enhancer-promoter
(SEQ ID NO: 310)
CCCACCTCCCTCTCTGTGCTGGGACTCACAGAGGGAGACCTCAGGAGGCAGTCTGTCCATCACA
TGTCCAAATGCAGAGCATACCCTGGGCTGGGCGCAGTGGCGCACAACTGTAATTCCAGCACTTT
GGGAGGCTGATGTGGAAGGATCACTTGAGCCCAGAAGTTCTAGACCAGCCTGGGCAACATGGCA
AGACCCTATCTCTACAAAAAAAGTTAAAAAATGAGCCACGTGTGGTGAGACACACCTGTAGTCC
CAGCTATTCAGGAGGCTGAGGTGAGGGGATCACTTAAGGCTGGGAGGTTGAGGCTGCAGTGAGT
CGTGGTTGCGCCACTGCACTCCAGCCTGGGCAACAGTGAGACCCTGTCTCAAAAGACAAAAAAA
AAAAAAAAAAAAAAAAGAACATATCCTGGTGTGGAGTAGGGGACGCTGCTCTGACAGAGGCTCG
GGGGCCTGAGCTGGCTCTGTGAGCTGGGGAGGAGGCAGACAGCCAGGCCTTGTCTGCAAGCAGA
CCTGGCAGCATTGGGCTGGCCGCCCCCCAGGGCCTCCTCTTCATGCCCAGTGAATGACTCACCT
TGGCACAGACACAATGTTCGGGGTGGGCACAGTGCCTGCTTCCCGCCGCACCCCAGCCCCCCTC
AAATGCCTTCCGAGAAGCCCATTGAGCAGGGGGCTTGCATTGCACCCCAGCCTGACAGCCTGGC
ATCTTGGGATAAAAGCAGCACAGCCCCCTAGGGGCTGCCCTTGCTGTGTGGCGCCACCGGCGGT
GGAGAACAAGGCTCTATTCAGCCTGTGCCCAGGAAAGGGGATCAGGGGATGCCCAGGCATGGAC
AGTGGGTGGCAGGGGGGGAGAGGAGGGCTGTCTGCTTCCCAGAAGTCCAAGGACACAAATGGGT
GAGGGGACTGGGCAGGGTTCTGACCCTGTGGGACCAGAGTGGAGGGCGTAGATGGACCTGAAGT
CTCCAGGGACAACAGGGCCCAGGTCTCAGGCTCCTAGTTGGGCCCAGTGGCTCCAGCGTTTCCA
AACCCATCCATCCCCAGAGGTTCTTCCCATCTCTCCAGGCTGATGTGTGGGAACTCGAGGAAAT
AAATCTCCAGTGGGAGACGGAGGGGTGGCCAGGGAAACGGGGCGCTGCAGGAATAAAGACGAGC
CAGCACAGCCAGCTCATGTGTAACGGCTTTGTGGAGCTGTCAAGGCCTGGTCTCTGGGAGAGAG
GCACAGGGAGGCCAGACAAGGAAGGGGTGACCTGGAGGGACAGATCCAGGGGCTAAAGTCCTGA
TAAGGCAAGAGAGTGCCGGCCCCCTCTTGCCCTATCAGGACCTCCACTGCCACATAGAGGCCAT
GATTGACCCTTAGACAAAGGGCTGGTGTCCAATCCCAGCCCCCAGCCCCAGAACTCCAGGGAAT
GAATGGGCAGAGAGCAGGAATGTGGGACATCTGTGTTCAAGGGAAGGACTCCAGGAGTCTGCTG
GGAATGAGGCCTAGTAGGAAATGAGGTGGCCCTTGAGGGTACAGAACAGGTTCATTCTTCGCCA
AATTCCCAGCACCTTGCAGGCACTTACAGCTGAGTGAGATAATGCCTGGGTTATGAAATCAAAA
AGTTGGAAAGCAGGTCAGAGGTCATCTGGTACAGCCCTTCCTTCCCTTTTTTTTTTTTTTTTTT
GTGAGACAAGGTCTCTCTCTGTTGCCCAGGCTGGAGTGGCGCAAACACAGCTCACTGCAGCCTC
AACCTACTGGGCTCAAGCAATCCTCCAGCCTCAGCCTCCCAAAGTGCTGGGATTACAAGCATGA
GCCACCCCACTCAGCCCTTTCCTTCCTTTTTAATTGATGCATAATAATTGTAAGTATTCATCAT
GGTCCAACCAACCCTTTCTTGACCCACCTTCCTAGAGAGAGGGTCCTCTTGCTTCAGCGGTCAG
GGCCCCAGACCCATGGTCTGGCTCCAGGTACCACCTGCCTCATGCAGGAGTTGGCGTGCCCAGG
AAGCTCTGCCTCTGGGCACAGTGACCTCAGTGGGGTGAGGGGAGCTCTCCCCATAGCTGGGCTG
CGGCCCAACCCCACCCCCTCAGGCTATGCCAGGGGGTGTTGCCAGGGGCACCCGGGCATCGCCA
GTCTAGCCCACTCCTTCATAAAGCCCTCGCATCCCAGGAGCGAGCAGAGCCAGAGCAGGTTGGA
GAGGAGACGCATCACCTCCGCTGCTCGC

vi. Enhancers and 5′ cap

In some instances, a construct can include a promoter sequence and/or an enhancer sequence. In some embodiments, an enhancer is a nucleotide sequence that can increase a level of transcription of a nucleic acid encoding a protein of interest (e.g., a KCNQ4 protein). In some embodiments, enhancer sequences (50-1500 base pairs in length) generally increase a level of transcription by providing additional binding sites for transcription-associated proteins (e.g., transcription factors). In some embodiments, an enhancer sequence is found within an intronic sequence. Unlike promoter sequences, enhancer sequences can act at much larger distance away from a transcription start site (e.g., as compared to a promoter). Non-limiting examples of enhancers include a RSV enhancer, a CMV enhancer, and a SV40 enhancer. An example of a CMV enhancer is described in, e.g., Boshart et al., Cell 41(2):521-530, 1985, which is incorporated in its entirety herein by reference.

As described herein, a 5′ cap (also termed an RNA cap, an RNA 7-methylguanosine cap or an RNA m.sup.7G cap) is a modified guanine nucleotide that has been added to a “front” or 5′ end of a eukaryotic messenger RNA shortly after a start of transcription. In some embodiments, a 5′ cap consists of a terminal group which is linked to a first transcribed nucleotide. Its presence is critical for recognition by a ribosome and protection from RNases. Cap addition is coupled to transcription, and occurs co-transcriptionally, such that each influences the other. Shortly after start of transcription, a 5′ end of an mRNA being synthesized is bound by a cap-synthesizing complex associated with RNA polymerase. This enzymatic complex catalyzes a chemical reactions that are required for mRNA capping. Synthesis proceeds as a multi-step biochemical reaction. A capping moiety can be modified to modulate functionality of mRNA such as its stability or efficiency of translation.

vii. Untranslated Regions (UTRs)

In some embodiments, any constructs described herein can include one or more untranslated regions. In some embodiments, a construct can include a 5′ UTR and/or a 3′ UTR. In some embodiments, if more than one UTR is present, UTRs may come from a single gene or more than one gene.

As is understood by those of skill in the art, an untranslated region (UTR) of a gene is transcribed but not translated. In some embodiments, a 5′ UTR starts at a transcription start site and continues to a start codon but does not include that start codon. In some embodiments, a 3′ UTR starts immediately following a stop codon and continues until a transcriptional termination signal. Without wishing to be bound by any particular theory, there is a growing body of evidence regarding regulatory roles played by UTRs in terms of stability of nucleic acid molecule and translation. In some embodiments, regulatory features of a UTR can be incorporated into any technologies (e.g., constructs, compositions, kits, or methods) as described herein to, e.g., enhance stability of a KCNQ4 protein.

For example, in some embodiments, a 5′ UTR is included in any constructs described herein. Non-limiting examples of 5′ UTRs including those from the following genes: albumin, serum amyloid A, Apolipoprotein A/B/E, transferrin, alpha fetoprotein, erythropoietin, and Factor VIII, can be used to enhance expression of a nucleic acid molecule, such as a mRNA. In some embodiments, 5′ UTRs have also been known, e.g., to form secondary structures that are involved in elongation factor binding.

In some embodiments, a 5′ UTR from an mRNA that is transcribed by a cell in a cochlea can be included in any technologies (e.g., constructs, compositions, kits, and methods) described herein.

In some embodiments, 3′ UTRs are known to have stretches of adenosines and uridines embedded in them. These AU-rich signatures are particularly prevalent in genes with high rates of turnover. Based on their sequence features and functional properties, AU-rich elements (AREs) can be separated into three classes (Chen et al., Mol. Cell. Biol. 15:5777-5788, 1995; Chen et al., Mol. Cell Biol. 15:2010-2018, 1995, each of which is incorporated in its entirety herein by reference): Class I AREs contain several dispersed copies of an AUUUA motif within U-rich regions. For example, c-Myc and MyoD mRNAs contain class I AREs. Class II AREs possess two or more overlapping UUAUUUA(U/A) (U/A) nonamers. GM-CSF and TNF-alpha mRNAs are examples that contain class II AREs. Class III AREs are less well defined. These U-rich regions do not contain an AUUUA motif. Two well-studied examples of this class are c-Jun and myogenin mRNAs.

Most proteins binding to AREs are known to destabilize a messenger, whereas members of the ELAV family, most notably HuR, have been documented to increase stability of mRNA. HuR binds to AREs of all three classes. Engineering HuR specific binding sites into a 3′ UTR of nucleic acid molecules will lead to HuR binding and thus, stabilization of a message in vivo.

In some embodiments, introduction, removal, or modification of 3′ UTR AREs can be used to modulate stability of an mRNA encoding a KCNQ4 protein (Kv7.4). In other embodiments, AREs can be removed or mutated to increase intracellular stability and thus increase translation and production of a KCNQ4 protein (Kv7.4).

In some embodiments, a UTR sequence is at least 85%, 90%, 95%, 98% or 99% identical to any UTR sequence disclosed herein (e.g., SEQ ID Nos: 25, 26, 27, 28, 29, and/or 30). By way of non-limiting example, untranslated regions may be or comprise the following:

HUMAN 5′ UTR SEQUENCE FROM ENDOGENOUS KCNQ4
TRANSCIRPT VARIANT 1
(SEQ ID NO: 25)
GACATGTGAGCGGCGCGCGCCGGTGGCAGGTGGAAAGGCGAGCGGCATGGAGCGCGTAATAAGA
GAGTTGGAGTCGGAAAGAGCAGCCCCAGTCGCCGGGGAAGCGGGAGGTCAGTGCGGGCTCCGGC
GGCCCCCAGGCTCCGAGCGCCCGCCCGCGGCCCCGGCCCGGCCCCTAGCCCCCGCCGCCCGCGC
CCGCCCCGGGTCGCCCCTCTGGCCCCGGGTCCGAGCCATGCGTCTCTGAGCGCCCCGAGCGCGC
CCCCGCCCCGGACCGTGCCCGGGCCCCGGCGCCCCCAGCCCGGCGCCGCCC
HUMAN 5′ UTR SEQUENCE FROM ENDOGENOUS KCNQ4
TRANSCIRPT VARIANT 2
(SEQ ID NO: 26)
GACATGTGAGCGGCGCGCGCCGGTGGCAGGTGGAAAGGCGAGCGGCATGGAGCGCGTAATAAGA
GAGTTGGAGTCGGAAAGAGCAGCCCCAGTCGCCGGGGAAGCGGGAGGTCAGTGCGGGCTCCGGC
GGCCCCCAGGCTCCGAGCGCCCGCCCGCGGCCCCGGCCCGGCCCCTAGCCCCCGCCGCCCGCGC
CCGCCCCGGGTCGCCCCTCTGGCCCCGGGTCCGAGCCATGCGTCTCTGAGCGCCCCGAGCGCGC
CCCCGCCCCGGACCGTGCCCGGGCCCCGGCGCCCCCAGCCCGGCGCCGCCC
HUMAN 5′ UTR SEQUENCE FROM ENDOGENOUS KCNQ4
TRANSCRIPT VARIANT X1
(SEQ ID NO: 27)
GACCACTCGTACCACAAAGTGGGCAGCTGAGGAGCTGATCACCGCCTGGTACATCGGGTTCCTG
GTGCTCATCTTCGCCTCCTTCCTGGTCTACCTGGCTGAGAAGGACGCCAACTCCGACTTCTCCT
CCTACGCCGACTCGCTCTGGTGGGGGACGATTACATTGACAACCATCGGCTATGGTGACAAGAC
ACCGCACACATGGCTGGGCAGGGTCCTGGCTGCTGGCTTCGCCTTACTGGGCATCTCTTTCTTT
GCCCTGCCTGCCGGCATCCTAGGCTCCGGCTTTGCCCTGAAGGTCCAGGAGCAGCACCGGCAGA
AGCACTTCGAGAAGCGGAGG
HUMAN 3′ UTR SEQUENCE FROM ENDOGENOUS KCNQ4
TRANSCIRPT VARIANT 1
(SEQ ID NO: 28)
GGGACTTCTCAGAGGCAGGGCAGCACACGGCCAGCCCCGCGGCCTGGCGCTCCGACTGCCCTCT
GAGGCCTCCGGACTCCTCTCGTACTTGAACTCACTCCCTCACGGGGAGAGAGACCACACGCAGT
ATTGAGCTGCCTGAGTGGGCGTGGTACCTGCTGTGGGTGCCAGCGCCCCTTCCCCACCTCAGGA
GCGTGAGATGCCAGGTCGCACAGAGGGCAGCAGCAGCGGCCGTCCCGCGGCCTCTGGGCCCCCC
AGTGCCCTGCCCACTCCATCAAGGCCCTATGTGGCCCACCTGGCAGGGGCACAGCCCCGGGAGT
GGGAGCGGGCGCTGGGGCCCTGGGCCCTGACCCAGCTTCCAGCTATGCAAGGTGAGGTCTCTGG
CCCACCCTTCGGACACAGCAGGGAAGCCCTCCCGCCAAGTCCCCGCCCCACTTGGGGGTGGGCC
AAGGTGCCCCCACAGGTACCCACAAAGCACAGGACCCTGCCACAAGGCAGGTGGACACCATATA
TGCAAACCATGTTAAATATGCAACTTTGGGGACCCCCATGGGGTCTCTCTGTCCCTCCCCCATT
GGGAGCTGGGCCCCCAGCAGTAGCTGGTCTCAGGCTGCTTGGCCACCACCCTGTCCCTATTCTT
TGGCTTATCACTCCTTCCCCTCCCAGCATGGGGCCTGTTTCTCCCCTGCCCTCTCCTAAGGGCA
ATGCCTGGGCCTTTCTTCCCATTTGCAAGTGTCAGCTCCCAGGGGCTCCCTCCTCCTGCTGGGT
GGCCACTCCCCTCCTTGGCCCTCCAGACACCACTCATAGTCAGCACAGGTTTCTGTATCCTCCC
CAAAACTCCCAGACAGTGCTTCGTGGAGGATCGCACAAACATAGCCTTTTAGTTTCTCCAGACA
GGAAGAAAGCCTCTCACACTTAAACATGCAATGACGTGACACACTTGGAGACATGAGTGCAGAG
CCACTCAGCCGCTCCTGGGCCTCTGCAGCAGATGCCAGTGGACTGGCCTTGCAGGGTGACGACC
ACTAAGAGGAAGACCCCCAACTCCATCTGAGCAGGAGAAGGAGCTTTGAAGTAACCCGAGAGCT
CTCCAGGCCCCACCCAGACCTTTACCCGCTCCCCTTCTTCAAGAAGATCTCCTCCTCTCTGGTC
CAGGAGCCCTAACCCACTGCCTCTGCCTGTCCCCAAGGGCCCGCCTCCGTGTCTCCACAGCACA
ACTCGGGCCCAGGCCTGACACCACTGGAGAGACCCCAGGCCCACTTCTAGCCAGGCCTGTGCCT
TCCTAGTCACTCTAACTCCCAGAGAGAATAAGAATGCATGTAATAGCTATACCAACCGCGCATC
CGGCTTTCACATGCACTGTCTCCCCTCCCTCCACACCCCACTTCTTCACTTCAATTGGCAGCGC
CACATCCAGGCGTCAGCCCCCATTCACTCCAGGAACACTTTCTTATCCCCACCCCTTTGCTCCT
CTTCTGCAAAGCCAATGCAGGTGGCAGGAAGGTGAGGGGTAGTGGACCAATGGCAACCCTCTGT
GGGAACAAGGGGCCGAGGCCACGCTGCCTGCATCTCGTGCTGGGGACCTGCATGCGCCAGCACC
AGGGCTTGGACTGGATCTTACTCAGTCCATGGTGCCCAGCCTCTGCCCCAACATGCCCTCTGCA
TGTGACCGTCATGCCCTGGATGGAGCCACTCCTGGCTCACCCCACCTGCACTGCACTGTCCCCA
GAGAGCCACCCCTCCACCCACTCAGAGACAGCTGTGGAGAGGGCCAGGAGAATGGGATTACCCT
ATGACCAAGGAGACATGGGAAGAAGCCCTCCTTCCTTCCACGATCGAGGTTCCGCCATCAACTC
GGTTCTCGGATATGCAAGTACCTCACTTTGTTAACTTATTAACTTATTGGTTTCATTAAAGTTT
TCAAGAGGA
HUMAN 3′ UTR SEQUENCE FROM ENDOGENOUS KCNQ4
TRANSCIRPT VARIANT 2
(SEQ ID NO: 29)
GGGACTTCTCAGAGGCAGGGCAGCACACGGCCAGCCCCGCGGCCTGGCGCTCCGACTGCCCTCT
GAGGCCTCCGGACTCCTCTCGTACTTGAACTCACTCCCTCACGGGGAGAGAGACCACACGCAGT
ATTGAGCTGCCTGAGTGGGCGTGGTACCTGCTGTGGGTGCCAGCGCCCCTTCCCCACCTCAGGA
GCGTGAGATGCCAGGTCGCACAGAGGGCAGCAGCAGCGGCCGTCCCGCGGCCTCTGGGCCCCCC
AGTGCCCTGCCCACTCCATCAAGGCCCTATGTGGCCCACCTGGCAGGGGCACAGCCCCGGGAGT
GGGAGCGGGCGCTGGGGCCCTGGGCCCTGACCCAGCTTCCAGCTATGCAAGGTGAGGTCTCTGG
CCCACCCTTCGGACACAGCAGGGAAGCCCTCCCGCCAAGTCCCCGCCCCACTTGGGGGTGGGCC
AAGGTGCCCCCACAGGTACCCACAAAGCACAGGACCCTGCCACAAGGCAGGTGGACACCATATA
TGCAAACCATGTTAAATATGCAACTTTGGGGACCCCCATGGGGTCTCTCTGTCCCTCCCCCATT
GGGAGCTGGGCCCCCAGCAGTAGCTGGTCTCAGGCTGCTTGGCCACCACCCTGTCCCTATTCTT
TGGCTTATCACTCCTTCCCCTCCCAGCATGGGGCCTGTTTCTCCCCTGCCCTCTCCTAAGGGCA
ATGCCTGGGCCTTTCTTCCCATTTGCAAGTGTCAGCTCCCAGGGGCTCCCTCCTCCTGCTGGGT
GGCCACTCCCCTCCTTGGCCCTCCAGACACCACTCATAGTCAGCACAGGTTTCTGTATCCTCCC
CAAAACTCCCAGACAGTGCTTCGTGGAGGATCGCACAAACATAGCCTTTTAGTTTCTCCAGACA
GGAAGAAAGCCTCTCACACTTAAACATGCAATGACGTGACACACTTGGAGACATGAGTGCAGAG
CCACTCAGCCGCTCCTGGGCCTCTGCAGCAGATGCCAGTGGACTGGCCTTGCAGGGTGACGACC
ACTAAGAGGAAGACCCCCAACTCCATCTGAGCAGGAGAAGGAGCTTTGAAGTAACCCGAGAGCT
CTCCAGGCCCCACCCAGACCTTTACCCGCTCCCCTTCTTCAAGAAGATCTCCTCCTCTCTGGTC
CAGGAGCCCTAACCCACTGCCTCTGCCTGTCCCCAAGGGCCCGCCTCCGTGTCTCCACAGCACA
ACTCGGGCCCAGGCCTGACACCACTGGAGAGACCCCAGGCCCACTTCTAGCCAGGCCTGTGCCT
TCCTAGTCACTCTAACTCCCAGAGAGAATAAGAATGCATGTAATAGCTATACCAACCGCGCATC
CGGCTTTCACATGCACTGTCTCCCCTCCCTCCACACCCCACTTCTTCACTTCAATTGGCAGCGC
CACATCCAGGCGTCAGCCCCCATTCACTCCAGGAACACTTTCTTATCCCCACCCCTTTGCTCCT
CTTCTGCAAAGCCAATGCAGGTGGCAGGAAGGTGAGGGGTAGTGGACCAATGGCAACCCTCTGT
GGGAACAAGGGGCCGAGGCCACGCTGCCTGCATCTCGTGCTGGGGACCTGCATGCGCCAGCACC
AGGGCTTGGACTGGATCTTACTCAGTCCATGGTGCCCAGCCTCTGCCCCAACATGCCCTCTGCA
TGTGACCGTCATGCCCTGGATGGAGCCACTCCTGGCTCACCCCACCTGCACTGCACTGTCCCCA
GAGAGCCACCCCTCCACCCACTCAGAGACAGCTGTGGAGAGGGCCAGGAGAATGGGATTACCCT
ATGACCAAGGAGACATGGGAAGAAGCCCTCCTTCCTTCCACGATCGAGGTTCCGCCATCAACTC
GGTTCTCGGATATGCAAGTACCTCACTTTGTTAACTTATTAACTTATTGGTTTCATTAAAGTTT
TCAAGAGGA
EXEMPLARY HUMAN 3′ UTR SEQUENCE FROM ENDOGENOUS
KCNQ4 TRANSCIRPT VARIANT X1
(SEQ ID NO: 30)
GGGACTTCTCAGAGGCAGGGCAGCACACGGCCAGCCCCGCGGCCTGGCGCTCCGACTGCCCTCT
GAGGCCTCCGGACTCCTCTCGTACTTGAACTCACTCCCTCACGGGGAGAGAGACCACACGCAGT
ATTGAGCTGCCTGAGTGGGCGTGGTACCTGCTGTGGGTGCCAGCGCCCCTTCCCCACCTCAGGA
GCGTGAGATGCCAGGTCGCACAGAGGGCAGCAGCAGCGGCCGTCCCGCGGCCTCTGGGCCCCCC
AGTGCCCTGCCCACTCCATCAAGGCCCTATGTGGCCCACCTGGCAGGGGCACAGCCCCGGGAGT
GGGAGCGGGCGCTGGGGCCCTGGGCCCTGACCCAGCTTCCAGCTATGCAAGGTGAGGTCTCTGG
CCCACCCTTCGGACACAGCAGGGAAGCCCTCCCGCCAAGTCCCCGCCCCACTTGGGGGTGGGCC
AAGGTGCCCCCACAGGTACCCACAAAGCACAGGACCCTGCCACAAGGCAGGTGGACACCATATA
TGCAAACCATGTTAAATATGCAACTTTGGGGACCCCCATGGGGTCTCTCTGTCCCTCCCCCATT
GGGAGCTGGGCCCCCAGCAGTAGCTGGTCTCAGGCTGCTTGGCCACCACCCTGTCCCTATTCTT
TGGCTTATCACTCCTTCCCCTCCCAGCATGGGGCCTGTTTCTCCCCTGCCCTCTCCTAAGGGCA
ATGCCTGGGCCTTTCTTCCCATTTGCAAGTGTCAGCTCCCAGGGGCTCCCTCCTCCTGCTGGGT
GGCCACTCCCCTCCTTGGCCCTCCAGACACCACTCATAGTCAGCACAGGTTTCTGTATCCTCCC
CAAAACTCCCAGACAGTGCTTCGTGGAGGATCGCACAAACATAGCCTTTTAGTTTCTCCAGACA
GGAAGAAAGCCTCTCACACTTAAACATGCAATGACGTGACACACTTGGAGACATGAGTGCAGAG
CCACTCAGCCGCTCCTGGGCCTCTGCAGCAGATGCCAGTGGACTGGCCTTGCAGGGTGACGACC
ACTAAGAGGAAGACCCCCAACTCCATCTGAGCAGGAGAAGGAGCTTTGAAGTAACCCGAGAGCT
CTCCAGGCCCCACCCAGACCTTTACCCGCTCCCCTTCTTCAAGAAGATCTCCTCCTCTCTGGTC
CAGGAGCCCTAACCCACTGCCTCTGCCTGTCCCCAAGGGCCCGCCTCCGTGTCTCCACAGCACA
ACTCGGGCCCAGGCCTGACACCACTGGAGAGACCCCAGGCCCACTTCTAGCCAGGCCTGTGCCT
TCCTAGTCACTCTAACTCCCAGAGAGAATAAGAATGCATGTAATAGCTATACCAACCGCGCATC
CGGCTTTCACATGCACTGTCTCCCCTCCCTCCACACCCCACTTCTTCACTTCAATTGGCAGCGC
CACATCCAGGCGTCAGCCCCCATTCACTCCAGGAACACTTTCTTATCCCCACCCCTTTGCTCCT
CTTCTGCAAAGCCAATGCAGGTGGCAGGAAGGTGAGGGGTAGTGGACCAATGGCAACCCTCTGT
GGGAACAAGGGGCCGAGGCCACGCTGCCTGCATCTCGTGCTGGGGACCTGCATGCGCCAGCACC
AGGGCTTGGACTGGATCTTACTCAGTCCATGGTGCCCAGCCTCTGCCCCAACATGCCCTCTGCA
TGTGACCGTCATGCCCTGGATGGAGCCACTCCTGGCTCACCCCACCTGCACTGCACTGTCCCCA
GAGAGCCACCCCTCCACCCACTCAGAGACAGCTGTGGAGAGGGCCAGGAGAATGGGATTACCCT
ATGACCAAGGAGACATGGGAAGAAGCCCTCCTTCCTTCCACGATCGAGGTTCCGCCATCAACTC
GGTTCTCGGATATGCAAGTACCTCACTTTGTTAACTTATTAACTTATTGGTTTCATTAAAGTTT
TCAAGAGGAGGTGA

viii. Kozak Sequences

In some embodiments, a construct of the present disclosure comprises one or more Kozak sequences. In some embodiments, natural 5′ UTRs include a sequence that plays a role in translation initiation. For example, in some embodiments, they harbor signatures like Kozak sequences, which are commonly known to be involved in a process by which a ribosome initiates translation of many genes. Kozak sequences generally have a consensus sequence CCR(A/G)CCAUGG, where R is a purine (A or G) three bases upstream of a start codon (AUG), which is followed by another “G”. In some embodiments, Kozak sequences may be included in synthetic or additional sequence elements, such as cloning sites.

ix. Internal Ribosome Entry Site (IRES)

In some embodiments, a construct of the present disclosure comprises one or more polynucleotide internal ribosome entry site (IRES). For example, in some embodiments, a construct of the present disclosure (e.g., a construct encoding a KCNQ4 gene product (e.g., human Kv7.4 protein, etc.) may include an IRES. In some embodiments, an IRES sequence is used to produce more than one polypeptide from a single gene transcript. In some embodiments, an IRES forms a complex secondary structure that allows translation initiation to occur from any position with an mRNA immediately downstream from where an IRES is located (see, e.g., Pelletier and Sonenberg, Mol. Cell. Biol. 8(3):1103-1112, 1988, which is incorporated in its entirety herein by reference).

There are several IRES sequences known to those skilled in the art, including those from, e.g., foot and mouth disease virus (FMDV), encephalomyocarditis virus (EMCV), human rhinovirus (HRV), cricket paralysis virus, human immunodeficiency virus (HIV), hepatitis A virus (HAV), hepatitis C virus (HCV), and poliovirus (PV). See e.g., Alberts, Molecular Biology of the Cell, Garland Science, 2002; and Hellen et al., Genes Dev. 15(13):1593-612, 2001, each of which is incorporated in its entirety herein by reference.

In some embodiments, an IRES sequence incorporated into a construct that encodes a KCNQ4 gene product (e.g., human Kv7.4 protein, etc.) or inhibitory nucleic acid (e.g., miRNA, etc.) thereof is foot and mouth disease virus (FMDV). Foot and Mouth Disease Virus 2A sequence is a small peptide (approximately 18 amino acids in length) that has been shown to mediate cleavage of polyproteins (Ryan, M D et al., EMBO 4:928-933, 1994; Mattion et al., J. Virology 70:8124-8127, 1996; Furler et al., Gene Therapy 8:864-873, 2001; and Halpin et al., Plant Journal 4:453-459, 1999, each of which is incorporated in its entirety herein by reference). Cleavage activity of the 2A sequence has previously been demonstrated in artificial systems including plasmids and gene therapy constructs (AAV and retroviruses) (Ryan et al., EMBO 4:928-933, 1994; Mattion et al., J. Virology 70:8124-8127, 1996; Furler et al., Gene Therapy 8:864-873, 2001; and Halpin et al., Plant Journal 4:453-459, 1999; de Felipe et al., Gene Therapy 6:198-208, 1999; de Felipe et al., Human Gene Therapy 11:1921-1931, 2000; and Klump et al., Gene Therapy 8:811-817, 2001, each of which is incorporated in its entirety herein by reference).

x. tRNA Sequences

In some embodiments, a construct of the present disclosure comprises a tRNA sequence. For instance, in some embodiments, a tRNA sequence may be used to facilitate a multiplex gRNA or shRNA/siRNA strategy. For example, in some embodiments, a tRNA may be included in a construct comprising at least 2, 3, 4, 5, 6, 7, 8, 9, 10 gRNAs; at least 2, 3, 4, 5, 6, 7, 8, 9, 10 shRNA/siRNAs etc. (see, e.g., PNAS 2015, 112 (11) 3570-3575, which is incorporated in its entirety herein by reference).

xi. Other Intronic Sequences

In some embodiments a construct of the present disclosure includes one or more intronic sequences, which intronic sequences do not comprise a UTR sequence. In some embodiments, non-UTR sequences may be incorporated into 5′ or 3′ UTRs. In some embodiments, introns or portions of intron sequences may be incorporated into t flanking regions of a polynucleotide in any constructs, compositions, kits, and methods provided herein. Incorporation of intronic sequences may increase protein production as well as mRNA levels. An intron can be an intron from a KCNQ4 gene or can be an intron from a heterologous gene, e.g., a hybrid adenovirus/mouse immunoglobulin intron (Yew et al., Human Gene Ter. 8(5):575-584, 1997, which is incorporated in its entirety herein by reference), an SV40 intron (Ostedgaard et al., Proc. Natl. Acad. Sci. U.S.A. 102(8):2952-2957, 2005, which is incorporated in its entirety herein by reference), an MVM intron (Wu et al., Mol. Ther. 16(2):280-289, 2008, which is incorporated in its entirety herein by reference), a factor IX truncated intron 1 (Wu et al., Mol. Ther. 16(2):280-289, 2008; Kurachi et al., J. Biol. Chem. 270(10):5276-5281, 1995, each of which is incorporated in its entirety herein by reference), a chimeric J-globulin splice donor/immunoglobulin heavy chain splice acceptor intron (Wu et al., Mol. Ther. 16(2):280-289, 2008; Choi et al., Mol. Brain 7:17, 2014, each of which is incorporated in its entirety herein by reference), SV40 late splice donor/splice acceptor intron (19S/16S) (Yew et al., Human Gene Ther. 8(5):575-584, 1997, which is incorporated in its entirety herein by reference), hybrid adenovirus spice donor/IgG splice acceptor (Choi et al., Mol. Brain 7:17, 1991; Huang and Gorman, Mol. Cell Biol. 10(4):1805-1810, 1990, each of which is incorporated in its entirety herein by reference). In some embodiments, an intronic sequence is at least 85%, 90%, 95%, 98% or 99% identical to any intronic sequence disclosed herein.

By way of non-limiting example, an intronic sequence in accordance with the present disclosure may be or comprise the following:

SD Intron
(SEQ ID NO: 31)
GTAAGTATCAAGGTTACAAGACAGGTTTAAGGAGA
CCAATAGAAACTGGGCTTGTCGAGACAGAGAAGAC
TCTTGCGTTTCT
SA Intron
(SEQ ID NO: 32)
GATAGGCACCTATTGGTCTTACTGACATCCACTTT
GCCTTTCTCTCCACAG

xii. Polyadenylation Sequences

In some embodiments, a construct of the present disclosure may comprise at least one poly(A) sequence. Most nascent eukaryotic mRNA possesses a poly(A) tail at its 3′ end which is added during a complex process that includes cleavage of a primary transcript and a coupled polyadenylation reaction (see, e.g., Proudfoot et al., Cell 108:501-512, 2002). A poly(A) tail confers mRNA stability and transferability (see, e.g., Molecular Biology of the Cell, Third Edition by B. Alberts et al., Garland Publishing, 1994). In some embodiments, a poly(A) sequence is positioned 3′ to a nucleic acid sequence encoding a KCNQ4 gene product or inhibitory nucleic acid molecule.

In some embodiments, polyadenylation refers to a covalent linkage of a polyadenylyl moiety, or its modified variant, to a messenger RNA molecule. In eukaryotic organisms, most messenger RNA (mRNA) molecules are polyadenylated at a 3′ end. In some embodiments, a 3′ poly(A) tail is a long sequence of adenine nucleotides (often several hundred) added to pre-mRNA through enzymatic action, polyadenylate polymerase. In higher eukaryotes, a poly(A) tail is added onto transcripts that contain a specific sequence, a polyadenylation signal. In some embodiments, a poly(A) tail and a protein bound to it aid in protecting mRNA from degradation by exonucleases. As will be understood to those of skill in the art, polyadenylation is also important for transcription termination, export of mRNA from a cell's nucleus, and translation. Polyadenylation occurs in a cell nucleus immediately after transcription of DNA into RNA, but additionally can also occur later in the cytoplasm. After transcription has been terminated, an mRNA chain is cleaved through action of an endonuclease complex associated with RNA polymerase. A cleavage site is usually characterized by the presence of a base sequence AAUAAA near a given cleavage site. After an mRNA has been cleaved, adenosine residues are added to the free 3′ end at the cleavage site.

In some embodiments, a poly(A) signal sequence is a sequence that triggers endonuclease cleavage of an mRNA and addition of a series of adenosines to the3′ end of a cleaved mRNA. A “poly(A)” portion refers to a series of adenosines attached by polyadenylation to an mRNA. In some embodiments of for the present disclosure, such as, e.g., transient expression, a polyA is between 50 and 5000 (SEQ ID NO: 93), preferably greater than 64, more preferably greater than 100, most preferably greater than 300 or 400. Poly(A) sequences can be modified chemically or enzymatically to modulate mRNA functionality such as localization, stability or efficiency of translation.

There are several poly(A) signal sequences that can be used, including those derived from bovine growth hormone (bgh) (Woychik et al., Proc. Natl. Acad. Sci. U.S.A. 81(13):3944-3948, 1984; U.S. Pat. No. 5,122,458; Yew et al., Human Gene Ther. 8(5):575-584, 1997; Xu et al., Human Gene Ther. 12(5):563-573, 2001; Xu et al., Gene Ther. 8:1323-1332, 2001; Wu et al., Mol. Ther. 16(2):280-289, 2008; Gray et al., Human Gene Ther. 22:1143-1153, 2011; Choi et al., Mol. Brain 7:17, 2014, each of which is incorporated in its entirety herein by reference), mouse-β-globin, mouse-α-globin (Orkin et al., EMBO J. 4(2):453-456, 1985; Thein et al., Blood 71(2):313-319, 1988, each which is incorporated in its entirety herein by reference), human collagen, polyoma virus (Batt et al., Mol. Cell Biol. 15(9):4783-4790, 1995, each of which is incorporated in its entirety herein by reference), Herpes simplex virus thymidine kinase gene (HSV TK), IgG heavy-chain gene polyadenylation signal (US 2006/0040354, which is incorporated in its entirety herein by reference), human growth hormone (hGH) (Szymanski et al., Mol. Therapy 15(7):1340-1347, 2007; Ostegaard et al., Proc. Natl. Acad. Sci. U.S.A. 102(8):2952-2957, 2005, each of which is incorporated in its entirety herein by reference), synthetic polyA (Levitt et al., Genes Dev. 3(7):1019-1025, 1989; Yew et al., Human Gene Ther. 8(5):575-584, 1997; Ostegaard et al., Proc. Natl. Acad. Sci. U.S.A. 102(8):2952-2957, 2005; Choi et al., Mol. Brain 7:17, 2014, each of which is incorporated in its entirety herein by reference), HIV-1 upstream poly(A) enhancer (Schambach et al., Mol. Ther. 15(6):1167-1173, 2007, each of which is incorporated in its entirety herein by reference), adenovirus (L3) upstream poly(A) enhancer (Schambach et al., Mol. Ther. 15(6):1167-1173, 2007, which is incorporated in its entirety herein by reference), hTHGB upstream poly(A) enhancer (Schambach et al., Mol. Ther. 15(6):1167-1173, 2007), hC2 upstream poly(A) enhancer (Schambach et al., Mol. Ther. 15(6):1167-1173, 2007), the group consisting of SV40 poly(A) signal sequence, such as the SV40 late and early poly(A) signal sequence (Schek et al., Mol. Cell Biol. 12(12):5386-5393, 1992; Choi et al., Mol. Brain 7:17, 2014; Schambach et al., Mol. Ther. 15(6):1167-1173, 2007, each of which is incorporated in its entirety herein by reference). Non-limiting examples of poly(A) signal sequences include SEQ ID NOs: 33, 34, or 35.

In some embodiments, a poly(A) signal sequence can be the sequence AATAAA. In some embodiments, an AATAAA sequence may be substituted with other hexanucleotide sequences with homology to AATAAA which are capable of signaling polyadenylation, including ATTAAA, AGTAAA, CATAAA, TATAAA, GATAAA, ACTAAA, AATATA, AAGAAA, AATAAT, AAAAAA, AATGAA, AATCAA, AACAAA, AATCAA, AATAAC, AATAGA, AATTAA, or AATAAG (see, e.g., WO 06/12414, which is incorporated in its entirety herein by reference).

In some embodiments, a poly(A) signal sequence can be a synthetic polyadenylation site (see, e.g., the pCl-neo expression construct of Promega which is based on Levitt el al, Genes Dev. 3(7):1019-1025, 1989, which is incorporated in its entirety herein by reference). In some embodiments, a poly(A) signal sequence is a polyadenylation signal of soluble neuropilin-1 (sNRP) (AAATAAAATACGAAATG (SEQ ID NO: 94)) (see, e.g., WO 05/073384, which is incorporated in its entirety herein by reference). In some embodiments, a poly(A) sequence is a bovine growth hormone poly(A) sequence. In some such embodiments, a bGH poly(A) sequence is or comprises SEQ ID NO: 36. In some embodiments, a construct or construct of the present disclosure comprises a bovine growth hormone polyA sequence represented by SEQ ID NO: 36. Additional examples of poly(A) signal sequences are known in the art.

In some embodiments, a polyA sequence is at least 85%, 90%, 95%, 98% or 99% identical to the polyA sequence of SEQ ID NOs: 33, 34, 35, or 36. In some embodiments, a polyadenylation sequence is at least 85%, 90%, 95%, 98% or 99% identical to any polyadenylation sequence disclosed herein.

By way of non-limiting example, a polyadenylation sequence may be or comprise:

Poly(A) signal sequence
(SEQ ID NO: 33)
GCTGATCAGCCTCGACTGTGCCTTCTAGTTGCCAG
CCATCTGTTGTTTGCCCCTCCCCCGTGCCTTCCTT
GACCCTGGAAGGTGCCACTCCCACTGTCCTTTCCT
AATAAAATGAGGAAATTGCATCGCATTGTCTGAGT
AGGTGTCATTCTATTCTGGGGGGTGGGGTGGGGCA
GGACAGCAAGGGGGAGGATTGGGAAGACAATAGCA
GGCATGCTGGGGATGCGGTGGGCTCTATGG
Poly(A) signal sequence
(SEQ ID NO: 34)
AATAAAATATCTTTATTTTCATTACATCTGTGTGT
TGGTTTTTTGTGTG
Poly(A) signal sequence
(SEQ ID NO: 35)
CGGCAATAAAAAGACAGAATAAAACGCACGGTGTT
GGGTCGTTTGTTC
bGHpA
(SEQ ID NO: 36)
CTGTGCCTTCTAGTTGCCAGCCATCTGTTGTTTGC
CCCTCCCCCGTGCCTTCCTTGACCCTGGAAGGTGC
CACTCCCACTGTCCTTTCCTAATAAAATGAGGAAA
TTGCATCGCATTGTCTGAGTAGGTGTCATTCTATT
CTGGGGGGTGGGGTGGGGCAGGACAGCAAGGGGGA
GGATTGGGAAGACAATAGCAGGCATGCTGGGGATG
CGGTGGGCTCTATGG

xiii. Other Regulatory Sequences

In some embodiments, a construct of the present disclosure can include one or more additional regulatory elements, e.g., a woodchuck hepatitis virus posttranscriptional regulatory element (WPRE), e.g., SEQ ID NO: 37. In some embodiments, a WPRE sequence is at least 85%, 90%, 95%, 98% or 99% identical to the WPRE sequence represented by SEQ ID NO: 37. In some embodiments, a regulatory element impacts expression of, e.g., a coding sequence of a construct (e.g., a sequence encoding a KCNQ4 gene product). In some embodiments, a regulatory element is a WPRE. In some such embodiments, such a regulatory element enhances or strengthens expression of one or more elements of a construct (e.g., a KCNQ4 gene product). By way of non-limiting example, a regulatory sequence may be or comprise the following:

WPRE element
(SEQ ID NO: 37)
AATCAACCTCTGGATTACAAAATTTGTGAAAGATT
GACTGGTATTCTTAACTATGTTGCTCCTTTTACGC
TATGTGGATACGCTGCTTTAATGCCTTTGTATCAT
GCTATTGCTTCCCGTATGGCTTTCATTTTCTCCTC
CTTGTATAAATCCTGGTTGCTGTCTCTTTATGAGG
AGTTGTGGCCCGTTGTCAGGCAACGTGGCGTGGTG
TGCACTGTGTTTGCTGACGCAACCCCCACTGGTTG
GGGCATTGCCACCACCTGTCAGCTCCTTTCCGGGA
CTTTCGCTTTCCCCCTCCCTATTGCCACGGCGGAA
CTCATCGCCGCCTGCCTTGCCCGCTGCTGGACAGG
GGCTCGGCTGTTGGGCACTGACAATTCCGTGGTGT
TGTCGGGGAAGCTGACGTCCTTTCCATGGCTGCTC
GCCTGTGTTGCCACCTGGATTCTGCGCGGGACGTC
CTTCTGCTACGTCCCTTCGGCCCTCAATCCAGCGG
ACCTTCCTTCCCGCGGCCTGCTGCCGGCTCTGCGG
CCTCTTCCGCGTCTTCGCCTTCGCCCTCAGACGAG
TCGGATCTCCCTTTGGGCCGCCTCCCCGC

xiv. Destabilization Domains

Any compositions (e.g., constructs) provided herein can optionally include a sequence that is or encodes a destabilization domain. In some embodiments, a destabilization domain is an amino acid sequence that decreases in vivo or in vitro half-life of a protein that includes a destabilization domain, e.g., as compared to the same protein lacking a stabilization domain. For example, in some embodiments, a destabilization domain may result in targeting of a protein that includes a destabilization domain for proteosomal degradation. Non-limiting examples of destabilization domains include a destabilizing domain of E. coli dihydrofolate reductase (DHFR) (Iwamoto et al. (2010) Chem. Biol. 17(9): 981-998, which is incorporated in its entirety herein by reference) and FK-506 binding protein (FKBP) (Wenlin et al. (2015) PLoS One 10(12): e0145783, which is incorporated in its entirety herein by reference). SEQ ID NO: 38 is an exemplary amino acid sequence of a DHFR destabilization domain. In some embodiments, a degradation sequence is at least 85%, 90%, 95%, 98% or 99% identical to the degradation sequence of SEQ ID NO: 38. Additional examples of destabilization domains are known in the art.

In some embodiments, any constructs provided herein can optionally include a degradation sequence, e.g., a CL1 degradation sequence of SEQ ID NO: 39. In some embodiments, a CL1 degradation sequence is at least 85%, 90%, 95%, 98% or 99% identical to the degradation sequence of SEQ ID NO: 39.

DHFR destabilization domain
(SEQ ID NO: 38)
ISLIAALAVDHVIGMETVMPWNLPADLAWFKRNTL
NKPVIMGRHTWESIGRPLPGRKNIILSSQPSTDDR
VTWVKSVDEAIAACGDVPEIMVIGGGRVYEQFLPK
AQKLYLTHIDAEVEGDTHFPDYEPDDWESVFSEFH
DADAQNSHSYCFEILERR
CL1
(SEQ ID NO: 39)
GCCTGCAAGAACTGGTTCAGCAGCCTGAGCCACTT
CGTGATCCACCTG

xv. Degron Domains

In some embodiments, any constructs provided herein can optionally include a C2H2 Zinc Finger “controllable” degron sequence and/or controllable destabilizing domain for a protein (e.g., a Cas9 protein). SEQ ID NO: 40 is an exemplary amino acid sequence of a C2H2 zinc finger degron domain.

C2H2 Zinc Finger Degron
(SEQ ID NO: 40)
FQCNQCGASFTQKGNLLRHIKLH

xvi. Reporter Sequences or Elements

Any constructs provided herein can optionally include a sequence encoding a reporter protein (“a reporter sequence”). For example, in some embodiments, a reporter sequence may be a FLAG, an eGFP, an mScarlet, a luciferase or any variant thereof. In some embodiments, a reporter sequence is visibly detectable without intervention. In some embodiments, a reporter element may be detected using a combination of fluorescent, histochemical, and/or transcript or protein analyses. Non-limiting examples of reporter sequences are described herein. Additional examples of reporter sequences are known in the art. In some embodiments, reporter sequence can be used to verify tissue-specific targeting capabilities and tissue-specific promoter regulatory activity of any constructs described herein.

In some embodiments, a reporter sequence is a FLAG tag (e.g., a 3xFLAG tag). In some embodiments, constructs or constructs of the present disclosure may comprise a 3×FLAG sequence. In some embodiments, presence of a reporter (e.g., of a construct carrying a FLAG tag in a mammalian cell (e.g., an inner ear cell, e.g., a cochlear hair or supporting cell) is detected by protein binding or detection assays (e.g., Western blots, immunohistochemistry, radioimmunoassay (RIA), mass spectrometry). An exemplary 3xFLAG tag sequence is provided as SEQ ID NO: 41.

3xFLAG tag sequence
(SEQ ID NO: 41)
GGATCCCGGGCTGACTACAAAGACCATGACGGTGA
TTATAAAGATCATGACATCGACTACAAGGATGAGG
ATGAGAAG

xvii. Additional Sequences

In some embodiments, constructs or constructs of the present disclosure may comprise a T2A element or sequence. In some embodiments, constructs of the present disclosure may include one or more cloning sites. In some such embodiments, cloning sites may not be fully removed prior to manufacturing for administration to a subject.

xviii. Single Construct Systems

In some embodiments, the present disclosure provides compositions which may include a single construct system. In some such embodiments, a single construct may deliver an inhibitory nucleic acid and/or a nucleic acid that encodes a functional (e.g., wild-type or otherwise functional, e.g., codon optimized) copy of KCNQ4. In some embodiments, a construct system is or comprises an AAV construct. In some embodiments described herein, a single AAV construct is capable of expressing a full-length KCNQ4 messenger RNA in a target cell. In some embodiments of any methods described herein, a single construct (e.g., any constructs described herein) that includes a sequence encoding a functional KCNQ4 protein (e.g., any construct that generates functional KCNQ4 protein) can be administered to a subject.

xix. Multiple Construct Systems

In some embodiments, a construct system of the present disclosure may comprise more than one construct (e.g., a dual or triple construct, e.g., for delivery of various components of a system provided by the present disclosure (e.g., a gene editing system and a transgene expression system). For example, in some embodiments, a dual construct may include two separate AAV constructs, each comprising a different component or construct (e.g., a CRISPR/Cas9 component and a replacement KCNQ4 component). In some embodiments, one construct (e.g., a first AAV construct) may comprise an inhibitory nucleic acid (e.g., an siRNA, a microRNA) and a second construct (e.g., a second AAV construct) may comprise a sequence encoding a functional KCNQ4 (e.g., a wild-type KCNQ4, e.g., a codon-optimized KCNQ4). In some embodiments, a dual AAV construct may include two separate AAV constructs, each including different or the same regulatory regions or promoters. In some embodiments, each construct comprises regulatory elements and promoters specific to a target, e.g., an ear cell, e.g., a hair cell, e.g., an outer hair cell.

In some embodiments, the present disclosure provides technologies that comprise multiple constructs. In some such embodiments, constructs may be all of a single construct type (e.g., AAV), or more than one type (e.g., AAV, adeno, etc.). In some such embodiments that comprise multiple constructs, each construct comprises a component of a system provided by the present disclosure, e.g., one construct comprises an inhibitory nucleic acid (e.g., a KCNQ4 miRNA) and another construct comprises a sequence encoding a functional KCNQ4 (e.g., a wild-type KCNQ4 gene that encodes a functional Kv7.4 protein, e.g., a codon-optimized KCNQ4 gene that encodes a functional Kv7.4 protein, etc.).

In some embodiments comprising multiple AAV constructs, AAV constructs may be of the same or different types, e.g., the same or different serotype.

In some embodiments, the present disclosure provides compositions comprising one or more constructs to deliver a therapeutic gene product or portion thereof to a subject in need thereof. For example, in some embodiments, a KCNQ4 gene is changed (e.g., via substitution, deletion, addition) in a genome of a subject. In some such embodiments, one or more constructs may be administered to a subject. In some such embodiments, one or more constructs may be administered to either (i) knockdown a variant (e.g., with a substitution, addition, or deletion) or nonfunctional (e.g., loss-of-function) KCNQ4 and/or (ii) provide a functional KCNQ4

xx. Exemplary Constructs

In some embodiments, the present disclosure provides technologies (e.g., compositions, systems, particles, comprising AAV-based constructs. In some embodiments, such technologies comprise a single construct. In some embodiments, such technologies comprise multiple constructs. In some embodiments, the present disclosure provides compositions or systems comprising multiple AAV particles each comprised of a single construct. In some embodiments, a single construct may deliver a polynucleotide that encodes a functional (e.g., wild type or otherwise functional, e.g., codon optimized) copy of a KCNQ4 gene. In some embodiments, a construct is or comprises an rAAV construct. In some embodiments described herein, a single rAAV construct is capable of expressing a full-length KCNQ4 messenger RNA or a characteristic protein thereof in a target cell (e.g., an inner ear cell). In some embodiments, a single construct (e.g., any of the constructs described herein) can include a sequence encoding a functional KCNQ4 protein (e.g., any construct that generates functional KCNQ4 protein). In some embodiments, a single construct (e.g., any of the constructs described herein) can include a sequence encoding a functional KCNQ4 protein (e.g., any construct that generates functional KCNQ4 protein) and optionally additional polypeptide sequences (e.g., regulatory sequences, and/or reporter sequences). In some embodiments, a single construct may deliver a polynucleotide that encodes a functional Cas9 protein. In some embodiments, a single construct may deliver a polynucleotide that encodes catalytically dead Cas9 protein. In some embodiments, a single construct may deliver a polynucleotide that encodes any number of miRNA, siRNA, shRNA, sgRNA, and/or associated regulatory regions.

In some embodiments, a single construct composition or system may comprise any or all of the exemplary construct components described herein. In some embodiments, an exemplary single construct is represented by SEQ ID NO: 172-291. In some embodiments, an exemplary single construct is at least 85%, 90%, 95%, 98% or 99% identical to the sequences represented by SEQ ID NO: 172-291. One skilled in the art would recognize that constructs may undergo additional modifications including codon-optimization, introduction of novel but functionally equivalent (e.g., silent mutations), addition of reporter sequences, and/or other routine modification.

### Exemplary Construct Embodiments

Thirty (30) miRNA Construct Sequences

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a microRNA backbone and KCNQ4 targeting sequence exemplified by SEQ ID NO: 104 engineered into the chimeric intron, optionally a cloning site exemplified by SEQ ID NO: 156, an eGFP coding region exemplified by SEQ ID NO: 103, optionally a cloning site exemplified by SEQ ID NO: 157, a microRNA backbone and KCNQ4 targeting sequence exemplified by SEQ ID NO: 104, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a microRNA backbone and KCNQ4 targeting sequence exemplified by SEQ ID NO: 105 engineered into the chimeric intron, optionally a cloning site exemplified by SEQ ID NO: 156, an eGFP coding region exemplified by SEQ ID NO: 103, optionally a cloning site exemplified by SEQ ID NO: 157, a microRNA backbone and KCNQ4 targeting sequence exemplified by SEQ ID NO: 105, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a microRNA backbone and KCNQ4 targeting sequence exemplified by SEQ ID NO: 106 engineered into the chimeric intron, optionally a cloning site exemplified by SEQ ID NO: 156, an eGFP coding region exemplified by SEQ ID NO: 103, optionally a cloning site exemplified by SEQ ID NO: 157, a microRNA backbone and KCNQ4 targeting sequence exemplified by SEQ ID NO: 106, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a microRNA backbone and KCNQ4 targeting sequence exemplified by SEQ ID NO: 104 engineered into the chimeric intron, optionally a cloning site exemplified by SEQ ID NO: 156, an eGFP coding region exemplified by SEQ ID NO: 103, optionally a cloning site exemplified by SEQ ID NO: 157, a microRNA backbone and KCNQ4 targeting sequence exemplified by SEQ ID NO: 107, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a microRNA backbone and KCNQ4 targeting sequence exemplified by SEQ ID NO: 105 engineered into the chimeric intron, optionally a cloning site exemplified by SEQ ID NO: 156, an eGFP coding region exemplified by SEQ ID NO: 103, optionally a cloning site exemplified by SEQ ID NO: 157, a microRNA backbone and KCNQ4 targeting sequence exemplified by SEQ ID NO: 108, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 106 with a microRNA backbone and KCNQ4 targeting sequence exemplified by SEQ ID NO: 104 engineered into the chimeric intron, optionally a cloning site exemplified by SEQ ID NO: 156, an eGFP coding region exemplified by SEQ ID NO: 103, optionally a cloning site exemplified by SEQ ID NO: 157, a microRNA backbone and KCNQ4 targeting sequence exemplified by SEQ ID NO: 109, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a microRNA backbone and KCNQ4 targeting sequence exemplified by SEQ ID NO: 104 engineered into the chimeric intron, optionally a cloning site exemplified by SEQ ID NO: 156, an eGFP coding region exemplified by SEQ ID NO: 103, optionally a cloning site exemplified by SEQ ID NO: 157, a microRNA backbone and KCNQ4 targeting sequence exemplified by SEQ ID NO: 110, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a microRNA backbone and KCNQ4 targeting sequence exemplified by SEQ ID NO: 105 engineered into the chimeric intron, optionally a cloning site exemplified by SEQ ID NO: 156, an eGFP coding region exemplified by SEQ ID NO: 103, optionally a cloning site exemplified by SEQ ID NO: 157, a microRNA backbone and KCNQ4 targeting sequence exemplified by SEQ ID NO: 111, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a microRNA backbone and KCNQ4 targeting sequence exemplified by SEQ ID NO: 106 engineered into the chimeric intron, optionally a cloning site exemplified by SEQ ID NO: 156, an eGFP coding region exemplified by SEQ ID NO: 103, optionally a cloning site exemplified by SEQ ID NO: 157, a microRNA backbone and KCNQ4 targeting sequence exemplified by SEQ ID NO: 112, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a microRNA backbone and KCNQ4 targeting sequence exemplified by SEQ ID NO: 104 engineered into the chimeric intron, optionally a cloning site exemplified by SEQ ID NO: 156, an eGFP coding region exemplified by SEQ ID NO: 103, optionally a cloning site exemplified by SEQ ID NO: 157, a microRNA backbone and KCNQ4 targeting sequence exemplified by SEQ ID NO: 113, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a microRNA backbone and KCNQ4 targeting sequence exemplified by SEQ ID NO: 105 engineered into the chimeric intron, optionally a cloning site exemplified by SEQ ID NO: 156, an eGFP coding region exemplified by SEQ ID NO: 103, optionally a cloning site exemplified by SEQ ID NO: 157, a microRNA backbone and KCNQ4 targeting sequence exemplified by SEQ ID NO: 114, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a microRNA backbone and KCNQ4 targeting sequence exemplified by SEQ ID NO: 106 engineered into the chimeric intron, optionally a cloning site exemplified by SEQ ID NO: 156, an eGFP coding region exemplified by SEQ ID NO: 103, optionally a cloning site exemplified by SEQ ID NO: 157, a microRNA backbone and KCNQ4 targeting sequence exemplified by SEQ ID NO: 115, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a microRNA backbone and KCNQ4 targeting sequence exemplified by SEQ ID NO: 107 engineered into the chimeric intron, optionally a cloning site exemplified by SEQ ID NO: 156, an eGFP coding region exemplified by SEQ ID NO: 103, optionally a cloning site exemplified by SEQ ID NO: 157, a microRNA backbone and KCNQ4 targeting sequence exemplified by SEQ ID NO: 107, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a microRNA backbone and KCNQ4 targeting sequence exemplified by SEQ ID NO: 108 engineered into the chimeric intron, optionally a cloning site exemplified by SEQ ID NO: 156, an eGFP coding region exemplified by SEQ ID NO: 103, optionally a cloning site exemplified by SEQ ID NO: 157, a microRNA backbone and KCNQ4 targeting sequence exemplified by SEQ ID NO: 108, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a microRNA backbone and KCNQ4 targeting sequence exemplified by SEQ ID NO: 109 engineered into the chimeric intron, optionally a cloning site exemplified by SEQ ID NO: 156, an eGFP coding region exemplified by SEQ ID NO: 103, optionally a cloning site exemplified by SEQ ID NO: 157, a microRNA backbone and KCNQ4 targeting sequence exemplified by SEQ ID NO: 109, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a microRNA backbone and KCNQ4 targeting sequence exemplified by SEQ ID NO: 107 engineered into the chimeric intron, optionally a cloning site exemplified by SEQ ID NO: 156, an eGFP coding region exemplified by SEQ ID NO: 103, optionally a cloning site exemplified by SEQ ID NO: 157, a microRNA backbone and KCNQ4 targeting sequence exemplified by SEQ ID NO: 110, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a microRNA backbone and KCNQ4 targeting sequence exemplified by SEQ ID NO: 108 engineered into the chimeric intron, optionally a cloning site exemplified by SEQ ID NO: 156, an eGFP coding region exemplified by SEQ ID NO: 103, optionally a cloning site exemplified by SEQ ID NO: 157, a microRNA backbone and KCNQ4 targeting sequence exemplified by SEQ ID NO: 111, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a microRNA backbone and KCNQ4 targeting sequence exemplified by SEQ ID NO: 109 engineered into the chimeric intron, optionally a cloning site exemplified by SEQ ID NO: 156, an eGFP coding region exemplified by SEQ ID NO: 103, optionally a cloning site exemplified by SEQ ID NO: 157, a microRNA backbone and KCNQ4 targeting sequence exemplified by SEQ ID NO: 112, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a microRNA backbone and KCNQ4 targeting sequence exemplified by SEQ ID NO: 107 engineered into the chimeric intron, optionally a cloning site exemplified by SEQ ID NO: 156, an eGFP coding region exemplified by SEQ ID NO: 103, optionally a cloning site exemplified by SEQ ID NO: 157, a microRNA backbone and KCNQ4 targeting sequence exemplified by SEQ ID NO: 113, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a microRNA backbone and KCNQ4 targeting sequence exemplified by SEQ ID NO: 108 engineered into the chimeric intron, optionally a cloning site exemplified by SEQ ID NO: 156, an eGFP coding region exemplified by SEQ ID NO: 103, optionally a cloning site exemplified by SEQ ID NO: 157, a microRNA backbone and KCNQ4 targeting sequence exemplified by SEQ ID NO: 114, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a microRNA backbone and KCNQ4 targeting sequence exemplified by SEQ ID NO: 109 engineered into the chimeric intron, optionally a cloning site exemplified by SEQ ID NO: 156, an eGFP coding region exemplified by SEQ ID NO: 103, optionally a cloning site exemplified by SEQ ID NO: 157, a microRNA backbone and KCNQ4 targeting sequence exemplified by SEQ ID NO: 114, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a microRNA backbone and KCNQ4 targeting sequence exemplified by SEQ ID NO: 110 engineered into the chimeric intron, optionally a cloning site exemplified by SEQ ID NO: 156, an eGFP coding region exemplified by SEQ ID NO: 103, optionally a cloning site exemplified by SEQ ID NO: 157, a microRNA backbone and KCNQ4 targeting sequence exemplified by SEQ ID NO: 110, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a microRNA backbone and KCNQ4 targeting sequence exemplified by SEQ ID NO: 111 engineered into the chimeric intron, optionally a cloning site exemplified by SEQ ID NO: 156, an eGFP coding region exemplified by SEQ ID NO: 103, optionally a cloning site exemplified by SEQ ID NO: 157, a microRNA backbone and KCNQ4 targeting sequence exemplified by SEQ ID NO: 111, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a microRNA backbone and KCNQ4 targeting sequence exemplified by SEQ ID NO: 112 engineered into the chimeric intron, optionally a cloning site exemplified by SEQ ID NO: 156, an eGFP coding region exemplified by SEQ ID NO: 103, optionally a cloning site exemplified by SEQ ID NO: 157, a microRNA backbone and KCNQ4 targeting sequence exemplified by SEQ ID NO: 112, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a microRNA backbone and KCNQ4 targeting sequence exemplified by SEQ ID NO: 110 engineered into the chimeric intron, optionally a cloning site exemplified by SEQ ID NO: 156, an eGFP coding region exemplified by SEQ ID NO: 103, optionally a cloning site exemplified by SEQ ID NO: 157, a microRNA backbone and KCNQ4 targeting sequence exemplified by SEQ ID NO: 113, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a microRNA backbone and KCNQ4 targeting sequence exemplified by SEQ ID NO: 111 engineered into the chimeric intron, optionally a cloning site exemplified by SEQ ID NO: 156, an eGFP coding region exemplified by SEQ ID NO: 103, optionally a cloning site exemplified by SEQ ID NO: 157, a microRNA backbone and KCNQ4 targeting sequence exemplified by SEQ ID NO: 114, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a microRNA backbone and KCNQ4 targeting sequence exemplified by SEQ ID NO: 112 engineered into the chimeric intron, optionally a cloning site exemplified by SEQ ID NO: 156, an eGFP coding region exemplified by SEQ ID NO: 103, optionally a cloning site exemplified by SEQ ID NO: 157, a microRNA backbone and KCNQ4 targeting sequence exemplified by SEQ ID NO: 115, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a microRNA backbone and KCNQ4 targeting sequence exemplified by SEQ ID NO: 113 engineered into the chimeric intron, optionally a cloning site exemplified by SEQ ID NO: 156, an eGFP coding region exemplified by SEQ ID NO: 103, optionally a cloning site exemplified by SEQ ID NO: 157, a microRNA backbone and KCNQ4 targeting sequence exemplified by SEQ ID NO: 113, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a microRNA backbone and KCNQ4 targeting sequence exemplified by SEQ ID NO: 114 engineered into the chimeric intron, optionally a cloning site exemplified by SEQ ID NO: 156, an eGFP coding region exemplified by SEQ ID NO: 103, optionally a cloning site exemplified by SEQ ID NO: 157, a microRNA backbone and KCNQ4 targeting sequence exemplified by SEQ ID NO: 114, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a microRNA backbone and KCNQ4 targeting sequence exemplified by SEQ ID NO: 115 engineered into the chimeric intron, optionally a cloning site exemplified by SEQ ID NO: 156, an eGFP coding region exemplified by SEQ ID NO: 103, optionally a cloning site exemplified by SEQ ID NO: 157, a microRNA backbone and KCNQ4 targeting sequence exemplified by SEQ ID NO: 115, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

Twenty (20) miRNA Construct Sequences and Background NNNNN Sequences

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a microRNA backbone and KCNQ4 targeting sequence exemplified by SEQ ID NO: 116 engineered into the chimeric intron, optionally a cloning site exemplified by SEQ ID NO: 156, an eGFP coding region exemplified by SEQ ID NO: 103, optionally a cloning site exemplified by SEQ ID NO: 157, a microRNA backbone and KCNQ4 targeting sequence exemplified by SEQ ID NO: 116, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a microRNA backbone and KCNQ4 targeting sequence exemplified by SEQ ID NO: 116 engineered into the chimeric intron, optionally a cloning site exemplified by SEQ ID NO: 156, an eGFP coding region exemplified by SEQ ID NO: 103, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 24, a chimeric intron exemplified by SEQ ID NO: 102, optionally a cloning site exemplified by SEQ ID NO: 156, an eGFP coding region exemplified by SEQ ID NO: 103, a microRNA backbone and KCNQ4 targeting sequence exemplified by SEQ ID NO: 134, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a microRNA backbone and KCNQ4 targeting sequence exemplified by SEQ ID NO: 117 engineered into the chimeric intron, optionally a cloning site exemplified by SEQ ID NO: 156, an eGFP coding region exemplified by SEQ ID NO: 103, optionally a cloning site exemplified by SEQ ID NO: 157, a microRNA backbone and KCNQ4 targeting sequence exemplified by SEQ ID NO: 117, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a microRNA backbone and KCNQ4 targeting sequence exemplified by SEQ ID NO: 118 engineered into the chimeric intron, optionally a cloning site exemplified by SEQ ID NO: 156, an eGFP coding region exemplified by SEQ ID NO: 103, optionally a cloning site exemplified by SEQ ID NO: 157, a microRNA backbone and KCNQ4 targeting sequence exemplified by SEQ ID NO: 118, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a microRNA backbone and KCNQ4 targeting sequence exemplified by SEQ ID NO: 119 engineered into the chimeric intron, optionally a cloning site exemplified by SEQ ID NO: 156, an eGFP coding region exemplified by SEQ ID NO: 103, optionally a cloning site exemplified by SEQ ID NO: 157, a microRNA backbone and KCNQ4 targeting sequence exemplified by SEQ ID NO: 119, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a microRNA backbone and KCNQ4 targeting sequence exemplified by SEQ ID NO: 120 engineered into the chimeric intron, optionally a cloning site exemplified by SEQ ID NO: 156, an eGFP coding region exemplified by SEQ ID NO: 103, optionally a cloning site exemplified by SEQ ID NO: 157, a microRNA backbone and KCNQ4 targeting sequence exemplified by SEQ ID NO: 120, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a microRNA backbone and KCNQ4 targeting sequence exemplified by SEQ ID NO: 121 engineered into the chimeric intron, optionally a cloning site exemplified by SEQ ID NO: 156, an eGFP coding region exemplified by SEQ ID NO: 103, optionally a cloning site exemplified by SEQ ID NO: 157, a microRNA backbone and KCNQ4 targeting sequence exemplified by SEQ ID NO: 121, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a microRNA backbone and KCNQ4 targeting sequence exemplified by SEQ ID NO: 122 engineered into the chimeric intron, optionally a cloning site exemplified by SEQ ID NO: 156, an eGFP coding region exemplified by SEQ ID NO: 103, optionally a cloning site exemplified by SEQ ID NO: 157, a microRNA backbone and KCNQ4 targeting sequence exemplified by SEQ ID NO: 122, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a microRNA backbone and KCNQ4 targeting sequence exemplified by SEQ ID NO: 123 engineered into the chimeric intron, optionally a cloning site exemplified by SEQ ID NO: 156, an eGFP coding region exemplified by SEQ ID NO: 103, optionally a cloning site exemplified by SEQ ID NO: 157, a microRNA backbone and KCNQ4 targeting sequence exemplified by SEQ ID NO: 123, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a microRNA backbone and KCNQ4 targeting sequence exemplified by SEQ ID NO: 124 engineered into the chimeric intron, optionally a cloning site exemplified by SEQ ID NO: 156, an eGFP coding region exemplified by SEQ ID NO: 103, optionally a cloning site exemplified by SEQ ID NO: 157, a microRNA backbone and KCNQ4 targeting sequence exemplified by SEQ ID NO: 124, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a microRNA backbone and KCNQ4 targeting sequence exemplified by SEQ ID NO: 125 engineered into the chimeric intron, optionally a cloning site exemplified by SEQ ID NO: 156, an eGFP coding region exemplified by SEQ ID NO: 103, optionally a cloning site exemplified by SEQ ID NO: 157, a microRNA backbone and KCNQ4 targeting sequence exemplified by SEQ ID NO: 125, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a microRNA backbone and KCNQ4 targeting sequence exemplified by SEQ ID NO: 126 engineered into the chimeric intron, optionally a cloning site exemplified by SEQ ID NO: 156, an eGFP coding region exemplified by SEQ ID NO: 103, optionally a cloning site exemplified by SEQ ID NO: 157, a microRNA backbone and KCNQ4 targeting sequence exemplified by SEQ ID NO: 126, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a microRNA backbone and KCNQ4 targeting sequence exemplified by SEQ ID NO: 127 engineered into the chimeric intron, optionally a cloning site exemplified by SEQ ID NO: 156, an eGFP coding region exemplified by SEQ ID NO: 103, optionally a cloning site exemplified by SEQ ID NO: 157, a microRNA backbone and KCNQ4 targeting sequence exemplified by SEQ ID NO: 127, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a microRNA backbone and KCNQ4 targeting sequence exemplified by SEQ ID NO: 128 engineered into the chimeric intron, optionally a cloning site exemplified by SEQ ID NO: 156, an eGFP coding region exemplified by SEQ ID NO: 103, optionally a cloning site exemplified by SEQ ID NO: 157, a microRNA backbone and KCNQ4 targeting sequence exemplified by SEQ ID NO: 128, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a microRNA backbone and KCNQ4 targeting sequence exemplified by SEQ ID NO: 129 engineered into the chimeric intron, optionally a cloning site exemplified by SEQ ID NO: 156, an eGFP coding region exemplified by SEQ ID NO: 103, optionally a cloning site exemplified by SEQ ID NO: 157, a microRNA backbone and KCNQ4 targeting sequence exemplified by SEQ ID NO: 129, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a microRNA backbone and KCNQ4 targeting sequence exemplified by SEQ ID NO: 130 engineered into the chimeric intron, optionally a cloning site exemplified by SEQ ID NO: 156, an eGFP coding region exemplified by SEQ ID NO: 103, optionally a cloning site exemplified by SEQ ID NO: 157, a microRNA backbone and KCNQ4 targeting sequence exemplified by SEQ ID NO: 130, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a microRNA backbone and KCNQ4 targeting sequence exemplified by SEQ ID NO: 131 engineered into the chimeric intron, optionally a cloning site exemplified by SEQ ID NO: 156, an eGFP coding region exemplified by SEQ ID NO: 103, optionally a cloning site exemplified by SEQ ID NO: 157, a microRNA backbone and KCNQ4 targeting sequence exemplified by SEQ ID NO: 131, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a microRNA backbone and KCNQ4 targeting sequence exemplified by SEQ ID NO: 132 engineered into the chimeric intron, optionally a cloning site exemplified by SEQ ID NO: 156, an eGFP coding region exemplified by SEQ ID NO: 103, optionally a cloning site exemplified by SEQ ID NO: 157, a microRNA backbone and KCNQ4 targeting sequence exemplified by SEQ ID NO: 132, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a microRNA backbone and KCNQ4 targeting sequence exemplified by SEQ ID NO: 133 engineered into the chimeric intron, optionally a cloning site exemplified by SEQ ID NO: 156, an eGFP coding region exemplified by SEQ ID NO: 103, optionally a cloning site exemplified by SEQ ID NO: 157, a microRNA backbone and KCNQ4 targeting sequence exemplified by SEQ ID NO: 133, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a microRNA backbone and KCNQ4 targeting sequence exemplified by SEQ ID NO: 133 engineered into the chimeric intron, optionally a cloning site exemplified by SEQ ID NO: 156, an eGFP coding region exemplified by SEQ ID NO: 103, optionally a cloning site exemplified by SEQ ID NO: 157, a microRNA backbone and KCNQ4 targeting sequence exemplified by SEQ ID NO: 133, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In some embodiments, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a microRNA backbone and KCNQ4 targeting sequence described herein engineered into the chimeric intron, optionally a cloning site exemplified by SEQ ID NO: 156, an eGFP coding region exemplified by SEQ ID NO: 103, optionally a cloning site exemplified by SEQ ID NO: 157, a microRNA backbone and KCNQ4 targeting sequence described herein, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a microRNA backbone and KCNQ4 targeting sequence described herein engineered into the chimeric intron, optionally a cloning site exemplified by SEQ ID NO: 156, an eGFP coding region exemplified by SEQ ID NO: 103, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 24, a chimeric intron exemplified by SEQ ID NO: 102, optionally a cloning site exemplified by SEQ ID NO: 156, an eGFP coding region exemplified by SEQ ID NO: 103, a microRNA backbone and KCNQ4 targeting sequence described herein, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In some embodiments, a microRNA backbone with a KCNQ4 targeting sequence occurs twice within the plasmid construct. For example, a microRNA backbone with a KCNQ4 targeting sequence can occur once in the 3′ untranslated region after the EGFP coding sequence. In some embodiments, a microRNA backbone with a KCNQ4 targeting sequence occurs once in the plasmid construct. For example, a microRNA backbone with a KCNQ4 targeting sequence can occur in the intron of the CAG promoter region. As another example, a microRNA backbone with a KCNQ4 targeting sequence can occur in the 3′ untranslated region after the EGFP coding sequence.

Eight (8) Luciferase and GFP Knockdown Constructs with shRNA Sequences

In one embodiment, an exemplary construct comprises: a 5′ ITR exemplified by SEQ ID NO: 19, optionally a cloning site exemplified by SEQ ID NO: 160, a CMV enhancer exemplified by SEQ ID NO: 98, a CMV promoter exemplified by SEQ ID NO: 99, optionally a cloning site exemplified by SEQ ID NO: 161, a luciferase coding sequence exemplified by SEQ ID NO: 140, optionally a cloning site exemplified by SEQ ID NO: 162, a T2A sequence exemplified by SEQ ID NO: 141, a turboGFP coding region exemplified by SEQ ID NO: 142, optionally a cloning site exemplified by SEQ ID NO: 163, an SV40 poly(A) site exemplified by SEQ ID NO: 143, optionally a cloning site exemplified by SEQ ID NO: 164, a U6 promoter sequence exemplified by SEQ ID NO: 144, an shRNA sequence described herein, a Pol III transcription termination sequence exemplified by SEQ ID NO: 149, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 160, a CMV enhancer exemplified by SEQ ID NO: 98, a CMV promoter exemplified by SEQ ID NO: 99, optionally a cloning site exemplified by SEQ ID NO: 161, a luciferase coding sequence exemplified by SEQ ID NO: 140, optionally a cloning site exemplified by SEQ ID NO: 162, a T2A sequence exemplified by SEQ ID NO: 141, a turboGFP coding region exemplified by SEQ ID NO: 142, optionally a cloning site exemplified by SEQ ID NO: 163, an SV40 poly(A) site exemplified by SEQ ID NO: 143, optionally a cloning site exemplified by SEQ ID NO: 164, a U6 promoter sequence exemplified by SEQ ID NO: 144, an shRNA sequence exemplified by SEQ ID NO: 48, a Pol III transcription termination sequence exemplified by SEQ ID NO: 149, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 160, a CMV enhancer exemplified by SEQ ID NO: 98, a CMV promoter exemplified by SEQ ID NO: 99, optionally a cloning site exemplified by SEQ ID NO: 161, a luciferase coding sequence exemplified by SEQ ID NO: 140, optionally a cloning site exemplified by SEQ ID NO: 162, a T2A sequence exemplified by SEQ ID NO: 141, a turboGFP coding region exemplified by SEQ ID NO: 142, optionally a cloning site exemplified by SEQ ID NO: 163, an SV40 poly(A) site exemplified by SEQ ID NO: 143, optionally a cloning site exemplified by SEQ ID NO: 164, a U6 promoter sequence exemplified by SEQ ID NO: 144, an shRNA sequence exemplified by SEQ ID NO: 49, a Pol III transcription termination sequence exemplified by SEQ ID NO: 149, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 160, a CMV enhancer exemplified by SEQ ID NO: 98, a CMV promoter exemplified by SEQ ID NO: 99, optionally a cloning site exemplified by SEQ ID NO: 161, a luciferase coding sequence exemplified by SEQ ID NO: 140, optionally a cloning site exemplified by SEQ ID NO: 162, a T2A sequence exemplified by SEQ ID NO: 141, a turboGFP coding region exemplified by SEQ ID NO: 142, optionally a cloning site exemplified by SEQ ID NO: 163, an SV40 poly(A) site exemplified by SEQ ID NO: 143, optionally a cloning site exemplified by SEQ ID NO: 164, a U6 promoter sequence exemplified by SEQ ID NO: 144, an shRNA sequence exemplified by SEQ ID NO: 50, a Pol III transcription termination sequence exemplified by SEQ ID NO: 149, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 160, a CMV enhancer exemplified by SEQ ID NO: 98, a CMV promoter exemplified by SEQ ID NO: 99, optionally a cloning site exemplified by SEQ ID NO: 161, a luciferase coding sequence exemplified by SEQ ID NO: 140, optionally a cloning site exemplified by SEQ ID NO: 162, a T2A sequence exemplified by SEQ ID NO: 141, a turboGFP coding region exemplified by SEQ ID NO: 142, optionally a cloning site exemplified by SEQ ID NO: 163, an SV40 poly(A) site exemplified by SEQ ID NO: 143, optionally a cloning site exemplified by SEQ ID NO: 164, a U6 promoter sequence exemplified by SEQ ID NO: 144, an shRNA sequence exemplified by SEQ ID NO: 51, a Pol III transcription termination sequence exemplified by SEQ ID NO: 149, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 160, a CMV enhancer exemplified by SEQ ID NO: 98, a CMV promoter exemplified by SEQ ID NO: 99, optionally a cloning site exemplified by SEQ ID NO: 161, a luciferase coding sequence exemplified by SEQ ID NO: 140, optionally a cloning site exemplified by SEQ ID NO: 162, a T2A sequence exemplified by SEQ ID NO: 141, a turboGFP coding region exemplified by SEQ ID NO: 142, optionally a cloning site exemplified by SEQ ID NO: 163, an SV40 poly(A) site exemplified by SEQ ID NO: 143, optionally a cloning site exemplified by SEQ ID NO: 164, a U6 promoter sequence exemplified by SEQ ID NO: 144, an shRNA sequence exemplified by SEQ ID NO: 52, a Pol III transcription termination sequence exemplified by SEQ ID NO: 149, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 160, a CMV enhancer exemplified by SEQ ID NO: 98, a CMV promoter exemplified by SEQ ID NO: 99, optionally a cloning site exemplified by SEQ ID NO: 161, a luciferase coding sequence exemplified by SEQ ID NO: 140, optionally a cloning site exemplified by SEQ ID NO: 162, a T2A sequence exemplified by SEQ ID NO: 141, a turboGFP coding region exemplified by SEQ ID NO: 142, optionally a cloning site exemplified by SEQ ID NO: 163, an SV40 poly(A) site exemplified by SEQ ID NO: 143, optionally a cloning site exemplified by SEQ ID NO: 164, a U6 promoter sequence exemplified by SEQ ID NO: 144, an shRNA sequence exemplified by SEQ ID NO: 53, a Pol III transcription termination sequence exemplified by SEQ ID NO: 149, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 160, a CMV enhancer exemplified by SEQ ID NO: 98, a CMV promoter exemplified by SEQ ID NO: 99, optionally a cloning site exemplified by SEQ ID NO: 161, a luciferase coding sequence exemplified by SEQ ID NO: 140, optionally a cloning site exemplified by SEQ ID NO: 162, a T2A sequence exemplified by SEQ ID NO: 141, a turboGFP coding region exemplified by SEQ ID NO: 142, optionally a cloning site exemplified by SEQ ID NO: 163, an SV40 poly(A) site exemplified by SEQ ID NO: 143, optionally a cloning site exemplified by SEQ ID NO: 164, a U6 promoter sequence exemplified by SEQ ID NO: 144, an shRNA sequence exemplified by SEQ ID NO: 54, a Pol III transcription termination sequence exemplified by SEQ ID NO: 149, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 160, a CMV enhancer exemplified by SEQ ID NO: 98, a CMV promoter exemplified by SEQ ID NO: 99, optionally a cloning site exemplified by SEQ ID NO: 161, a luciferase coding sequence exemplified by SEQ ID NO: 140, optionally a cloning site exemplified by SEQ ID NO: 162, a T2A sequence exemplified by SEQ ID NO: 141, a turboGFP coding region exemplified by SEQ ID NO: 142, optionally a cloning site exemplified by SEQ ID NO: 163, an SV40 poly(A) site exemplified by SEQ ID NO: 143, optionally a cloning site exemplified by SEQ ID NO: 164, a U6 promoter sequence exemplified by SEQ ID NO: 144, an shRNA sequence exemplified by SEQ ID NO: 55, a Pol III transcription termination sequence exemplified by SEQ ID NO: 149, optionally a cloning site exemplified by SEQ ID NO: 159, and a 3′ ITR exemplified by SEQ ID NO: 20.

Two (2) Wild-Type hKCNQ4 Construct Sequences

In one embodiment, an exemplary construct comprises: a 5′ ITR exemplified by SEQ ID NO: 19, optionally a cloning site exemplified by SEQ ID NO: 160, a CMV enhancer exemplified by SEQ ID NO: 98, a CMV promoter exemplified by SEQ ID NO: 99, optionally a cloning site exemplified by SEQ ID NO: 161, mKCNQ4 wild-type coding sequence exemplified by SEQ ID NO: 91, optionally a cloning site exemplified by SEQ ID NO: 165, a 3×Flag tag sequence exemplified by SEQ ID NO: 145, optionally a cloning site exemplified by SEQ ID NO: 162, a T2A sequence exemplified by SEQ ID NO: 141, an mScarlet coding region exemplified by SEQ ID NO: 146, optionally a cloning site exemplified by SEQ ID NO: 166, a bGHpoly(a) signal exemplified by SEQ ID NO: 36, optionally a cloning site exemplified by SEQ ID NO: 159, and a 3′ ITR exemplified by SEQ ID NO: 18.

In one embodiment, an exemplary construct comprises: a 5′ ITR exemplified by SEQ ID NO: 19, optionally a cloning site exemplified by SEQ ID NO: 160, a CMV enhancer exemplified by SEQ ID NO: 98, a CMV promoter exemplified by SEQ ID NO: 99, optionally a cloning site exemplified by SEQ ID NO: 161, hKCNQ4 wild-type coding sequence exemplified by SEQ ID NO: 90, optionally a cloning site exemplified by SEQ ID NO: 165, a 3×Flag tag sequence exemplified by SEQ ID NO: 145, optionally a cloning site exemplified by SEQ ID NO: 162, a T2A sequence exemplified by SEQ ID NO: 141, an mScarlet coding region exemplified by SEQ ID NO: 146, optionally a cloning site exemplified by SEQ ID NO: 166, a bGHpoly(a) signal exemplified by SEQ ID NO: 36, optionally a cloning site exemplified by SEQ ID NO: 159, and a 3′ ITR exemplified by SEQ ID NO: 18.

Five (5) Codon Optimized hKCNQ4 Construct Sequences

In one embodiment, an exemplary construct comprises: a 5′ ITR exemplified by SEQ ID NO: 19, optionally a cloning site exemplified by SEQ ID NO: 160, a CMV enhancer exemplified by SEQ ID NO: 98, a CMV promoter exemplified by SEQ ID NO: 99, optionally a cloning site exemplified by SEQ ID NO: 161, a hKCNQ4 codon optimized coding sequence exemplified by SEQ ID NO: 9, optionally a cloning site exemplified by SEQ ID NO: 165, a 3xFlag tag sequence exemplified by SEQ ID NO: 145, optionally a cloning site exemplified by SEQ ID NO: 162, a T2A sequence exemplified by SEQ ID NO: 141, a mScarlet coding region exemplified by SEQ ID NO: 146, optionally a cloning site exemplified by SEQ ID NO: 166, a bGHpoly(a) signal exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 160, a CMV enhancer exemplified by SEQ ID NO: 98, a CMV promoter exemplified by SEQ ID NO: 99, optionally a cloning site exemplified by SEQ ID NO: 161, a hKCNQ4 codon optimized coding sequence exemplified by SEQ ID NO: 10, optionally a cloning site exemplified by SEQ ID NO: 165, a 3xFlag tag sequence exemplified by SEQ ID NO: 145, optionally a cloning site exemplified by SEQ ID NO: 162, a T2A sequence exemplified by SEQ ID NO: 141, a mScarlet coding region exemplified by SEQ ID NO: 146, optionally a cloning site exemplified by SEQ ID NO: 166, a bGHpoly(a) signal exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 160, a CMV enhancer exemplified by SEQ ID NO: 98, a CMV promoter exemplified by SEQ ID NO: 100, optionally a cloning site exemplified by SEQ ID NO: 161, a hKCNQ4 codon optimized coding sequence exemplified by SEQ ID NO: 9, optionally a cloning site exemplified by SEQ ID NO: 166, a bGHpoly(a) signal exemplified by SEQ ID NO: 36, optionally a cloning site exemplified by SEQ ID NO: 164, a U6 promoter sequence exemplified by SEQ ID NO: 144, a guide RNA sequence exemplified by SEQ ID NO: 42, a tcrRNA sequence exemplified by SEQ ID NO: 147, a Pol III transcription termination sequence exemplified by SEQ ID NO: 149, optionally a cloning site exemplified by SEQ ID NO: 167, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 160, a CMV enhancer exemplified by SEQ ID NO: 98, a CMV promoter exemplified by SEQ ID NO: 100, optionally a cloning site exemplified by SEQ ID NO: 161, a hKCNQ4 codon optimized coding sequence exemplified by SEQ ID NO: 9, optionally a cloning site exemplified by SEQ ID NO: 166, a bGHpoly(a) signal exemplified by SEQ ID NO: 36, optionally a cloning site exemplified by SEQ ID NO: 164, a U6 promoter sequence exemplified by SEQ ID NO: 144, a guide RNA sequence exemplified by SEQ ID NO: 43, a tcrRNA sequence exemplified by SEQ ID NO: 147, a Pol III transcription termination sequence exemplified by SEQ ID NO: 149, optionally a cloning site exemplified by SEQ ID NO: 167, and a 3′ ITR exemplified by SEQ ID NO: 20

In one embodiment, an exemplary construct comprises: a 5′ ITR exemplified by SEQ ID NO: 19, optionally a cloning site exemplified by SEQ ID NO: 160, a CMV enhancer exemplified by SEQ ID NO: 98, a CMV promoter exemplified by SEQ ID NO: 100, optionally a cloning site exemplified by SEQ ID NO: 161, a hKCNQ4 codon optimized coding sequence exemplified by SEQ ID NO: 9, optionally a cloning site exemplified by SEQ ID NO: 166, a bGHpoly(a) signal exemplified by SEQ ID NO: 36, optionally a cloning site exemplified by SEQ ID NO: 164, a U6 promoter sequence exemplified by SEQ ID NO: 144, a guide RNA sequence exemplified by SEQ ID NO: 150, a tcrRNA sequence exemplified by SEQ ID NO: 147, a Pol III transcription termination sequence exemplified by SEQ ID NO: 149, optionally a cloning site exemplified by SEQ ID NO: 167, and a 3′ ITR exemplified by SEQ ID NO: 20

### Four (4) Cas9 Construct Sequences

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 98, a CMV promoter exemplified by SEQ ID NO: 99, optionally a cloning site exemplified by SEQ ID NO: 161, an SV40 nuclear localization signal exemplified by SEQ ID NO: 151, optionally a cloning site exemplified by SEQ ID NO: 168, a catalytically-inactive saCas9 coding sequence exemplified by SEQ ID NO: 152, optionally a cloning site exemplified by SEQ ID NO: 169, an SV40 nuclear localization signal exemplified by SEQ ID NO: 153, optionally a cloning site exemplified by SEQ ID NO: 166, an SV40 poly(A) signal sequence exemplified by SEQ ID NO: 143, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV promoter exemplified by SEQ ID NO: 99, optionally a cloning site exemplified by SEQ ID NO: 161, an SV40 nuclear localization signal exemplified by SEQ ID NO: 151, optionally a cloning site exemplified by SEQ ID NO: 168, a catalytically-inactive saCas9 coding sequence exemplified by SEQ ID NO: 152, optionally a cloning site exemplified by SEQ ID NO: 169, an SV40 nuclear localization signal exemplified by SEQ ID NO: 153, optionally a cloning site exemplified by SEQ ID NO: 166, an SV40 poly(A) signal sequence exemplified by SEQ ID NO: 143, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 98, a CMV promoter exemplified by SEQ ID NO: 99, optionally a cloning site exemplified by SEQ ID NO: 161, an SV40 nuclear localization signal exemplified by SEQ ID NO: 151, optionally a cloning site exemplified by SEQ ID NO: 168, a catalytically active saCas9 coding sequence exemplified by SEQ ID NO: 154, optionally a cloning site exemplified by SEQ ID NO: 169, an SV40 nuclear localization signal exemplified by SEQ ID NO: 153, optionally a cloning site exemplified by SEQ ID NO: 166, an SV40 poly(A) signal sequence exemplified by SEQ ID NO: 143, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV promoter exemplified by SEQ ID NO: 99, optionally a cloning site exemplified by SEQ ID NO: 161, an SV40 nuclear localization signal exemplified by SEQ ID NO: 151, optionally a cloning site exemplified by SEQ ID NO: 168, a catalytically saCas9 coding sequence exemplified by SEQ ID NO: 154, optionally a cloning site exemplified by SEQ ID NO: 169, an SV40 nuclear localization signal exemplified by SEQ ID NO: 153, optionally a cloning site exemplified by SEQ ID NO: 166, an SV40 poly(A) signal sequence exemplified by SEQ ID NO: 143, optionally a cloning site exemplified by SEQ ID NO: 159, and a 3′ ITR exemplified by SEQ ID NO: 20.

Eight (8) Cas9 with eGFP and sgRNA Construct Sequences

In one embodiment, an exemplary construct comprises: a 5′ ITR exemplified by SEQ ID NO: 19, optionally a cloning site exemplified by SEQ ID NO: 160, a CMV enhancer exemplified by SEQ ID NO: 98, a CMV promoter exemplified by SEQ ID NO: 99, optionally a cloning site exemplified by SEQ ID NO: 161, an SV40 nuclear localization signal exemplified by SEQ ID NO: 151, optionally a cloning site exemplified by SEQ ID NO: 168, a catalytically active saCas9 coding sequence exemplified by SEQ ID NO: 154, optionally a cloning site exemplified by SEQ ID NO: 169, an SV40 nuclear localization signal exemplified by SEQ ID NO: 153, optionally a cloning site exemplified by SEQ ID NO: 162, a T2A sequence exemplified by SEQ ID NO: 141, a turboGFP sequence exemplified by SEQ ID NO: 142, optionally a cloning site exemplified by SEQ ID NO: 158, a SV-40 poly(A) signal sequence exemplified by SEQ ID NO: 143, optionally a cloning site exemplified by SEQ ID NO: 164, a U6 promoter sequence exemplified by SEQ ID NO: 144, a guide RNA sequence exemplified by SEQ ID NO: 43, a tcrRNA sequence exemplified by SEQ ID NO: 147, a Pol III transcription termination sequence exemplified by SEQ ID NO: 149, optionally a cloning site exemplified by SEQ ID NO: 170, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 160, a CMV enhancer exemplified by SEQ ID NO: 98, a CMV promoter exemplified by SEQ ID NO: 99, optionally a cloning site exemplified by SEQ ID NO: 161, an SV40 nuclear localization signal exemplified by SEQ ID NO: 151, optionally a cloning site exemplified by SEQ ID NO: 168, a catalytically-inactive saCas9 coding sequence exemplified by SEQ ID NO: 152, optionally a cloning site exemplified by SEQ ID NO: 169, an SV40 nuclear localization signal exemplified by SEQ ID NO: 153, optionally a cloning site exemplified by SEQ ID NO: 162, a T2A sequence exemplified by SEQ ID NO: 141, a turboGFP sequence exemplified by SEQ ID NO: 142, optionally a cloning site exemplified by SEQ ID NO: 158, a SV-40 poly(A) signal sequence exemplified by SEQ ID NO: 143, optionally a cloning site exemplified by SEQ ID NO: 164, a U6 promoter sequence exemplified by SEQ ID NO: 144, a guide RNA sequence exemplified by SEQ ID NO: 43, a tcrRNA sequence exemplified by SEQ ID NO: 147, a Pol III transcription termination sequence exemplified by SEQ ID NO: 149, optionally a cloning site exemplified by SEQ ID NO: 170, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 160, a CMV enhancer exemplified by SEQ ID NO: 98, a CMV promoter exemplified by SEQ ID NO: 99, optionally a cloning site exemplified by SEQ ID NO: 161, an SV40 nuclear localization signal exemplified by SEQ ID NO: 151, optionally a cloning site exemplified by SEQ ID NO: 168, a catalytically active saCas9 coding sequence exemplified by SEQ ID NO: 154, optionally a cloning site exemplified by SEQ ID NO: 169, an SV40 nuclear localization signal exemplified by SEQ ID NO: 153, optionally a cloning site exemplified by SEQ ID NO: 162, a T2A sequence exemplified by SEQ ID NO: 141, a turboGFP sequence exemplified by SEQ ID NO: 142, optionally a cloning site exemplified by SEQ ID NO: 158, a SV-40 poly(A) signal sequence exemplified by SEQ ID NO: 143, optionally a cloning site exemplified by SEQ ID NO: 164, a U6 promoter sequence exemplified by SEQ ID NO: 144, a guide RNA sequence exemplified by SEQ ID NO: 42, a tcrRNA sequence exemplified by SEQ ID NO: 147, a Pol III transcription termination sequence exemplified by SEQ ID NO: 149, optionally a cloning site exemplified by SEQ ID NO: 170, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 160, a CMV enhancer exemplified by SEQ ID NO: 98, a CMV promoter exemplified by SEQ ID NO: 99, optionally a cloning site exemplified by SEQ ID NO: 161, an SV40 nuclear localization signal exemplified by SEQ ID NO: 151, optionally a cloning site exemplified by SEQ ID NO: 168, a catalytically-inactive saCas9 coding sequence exemplified by SEQ ID NO: 152, optionally a cloning site exemplified by SEQ ID NO: 169, an SV40 nuclear localization signal exemplified by SEQ ID NO: 153, optionally a cloning site exemplified by SEQ ID NO: 162, a T2A sequence exemplified by SEQ ID NO: 141, a turboGFP sequence exemplified by SEQ ID NO: 142, optionally a cloning site exemplified by SEQ ID NO: 158, a SV-40 poly(A) signal sequence exemplified by SEQ ID NO: 143, optionally a cloning site exemplified by SEQ ID NO: 164, a U6 promoter sequence exemplified by SEQ ID NO: 144, a guide RNA sequence exemplified by SEQ ID NO: 42, a tcrRNA sequence exemplified by SEQ ID NO: 147, a Pol III transcription termination sequence exemplified by SEQ ID NO: 149, optionally a cloning site exemplified by SEQ ID NO: 170, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 160, a CMV enhancer exemplified by SEQ ID NO: 98, a CMV promoter exemplified by SEQ ID NO: 99, optionally a cloning site exemplified by SEQ ID NO: 161, an SV40 nuclear localization signal exemplified by SEQ ID NO: 151, optionally a cloning site exemplified by SEQ ID NO: 168, a catalytically active saCas9 coding sequence exemplified by SEQ ID NO: 154, optionally a cloning site exemplified by SEQ ID NO: 169, an SV40 nuclear localization signal exemplified by SEQ ID NO: 153, optionally a cloning site exemplified by SEQ ID NO: 162, a T2A sequence exemplified by SEQ ID NO: 141, a turboGFP sequence exemplified by SEQ ID NO: 142, optionally a cloning site exemplified by SEQ ID NO: 158, a SV-40 poly(A) signal sequence exemplified by SEQ ID NO: 143, optionally a cloning site exemplified by SEQ ID NO: 164, a U6 promoter sequence exemplified by SEQ ID NO: 144, a guide RNA sequence exemplified by SEQ ID NO: 44, a tcrRNA sequence exemplified by SEQ ID NO: 147, a Pol III transcription termination sequence exemplified by SEQ ID NO: 149, optionally a cloning site exemplified by SEQ ID NO: 170, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 160, a CMV enhancer exemplified by SEQ ID NO: 98, a CMV promoter exemplified by SEQ ID NO: 99, optionally a cloning site exemplified by SEQ ID NO: 161, an SV40 nuclear localization signal exemplified by SEQ ID NO: 151, optionally a cloning site exemplified by SEQ ID NO: 168, a catalytically active saCas9 coding sequence exemplified by SEQ ID NO: 154, optionally a cloning site exemplified by SEQ ID NO: 169, an SV40 nuclear localization signal exemplified by SEQ ID NO: 153, optionally a cloning site exemplified by SEQ ID NO: 162, a T2A sequence exemplified by SEQ ID NO: 141, a turboGFP sequence exemplified by SEQ ID NO: 142, optionally a cloning site exemplified by SEQ ID NO: 158, a SV-40 poly(A) signal sequence exemplified by SEQ ID NO: 143, optionally a cloning site exemplified by SEQ ID NO: 164, a U6 promoter sequence exemplified by SEQ ID NO: 144, a guide RNA sequence exemplified by SEQ ID NO: 45, a tcrRNA sequence exemplified by SEQ ID NO: 147, a Pol III transcription termination sequence exemplified by SEQ ID NO: 149, optionally a cloning site exemplified by SEQ ID NO: 170, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 160, a CMV enhancer exemplified by SEQ ID NO: 98, a CMV promoter exemplified by SEQ ID NO: 99, optionally a cloning site exemplified by SEQ ID NO: 161, an SV40 nuclear localization signal exemplified by SEQ ID NO: 151, optionally a cloning site exemplified by SEQ ID NO: 168, a catalytically active saCas9 coding sequence exemplified by SEQ ID NO: 154, optionally a cloning site exemplified by SEQ ID NO: 169, an SV40 nuclear localization signal exemplified by SEQ ID NO: 153, optionally a cloning site exemplified by SEQ ID NO: 162, a T2A sequence exemplified by SEQ ID NO: 141, a turboGFP sequence exemplified by SEQ ID NO: 142, optionally a cloning site exemplified by SEQ ID NO: 158, a SV-40 poly(A) signal sequence exemplified by SEQ ID NO: 143, optionally a cloning site exemplified by SEQ ID NO: 164, a U6 promoter sequence exemplified by SEQ ID NO: 144, a guide RNA sequence exemplified by SEQ ID NO: 46, a tcrRNA sequence exemplified by SEQ ID NO: 147, a Pol III transcription termination sequence exemplified by SEQ ID NO: 149, optionally a cloning site exemplified by SEQ ID NO: 170, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 160, a CMV enhancer exemplified by SEQ ID NO: 98, a CMV promoter exemplified by SEQ ID NO: 99, optionally a cloning site exemplified by SEQ ID NO: 161, an SV40 nuclear localization signal exemplified by SEQ ID NO: 151, optionally a cloning site exemplified by SEQ ID NO: 168, a catalytically active saCas9 coding sequence exemplified by SEQ ID NO: 154, optionally a cloning site exemplified by SEQ ID NO: 169, an SV40 nuclear localization signal exemplified by SEQ ID NO: 153, optionally a cloning site exemplified by SEQ ID NO: 162, a T2A sequence exemplified by SEQ ID NO: 141, a turboGFP sequence exemplified by SEQ ID NO: 142, optionally a cloning site exemplified by SEQ ID NO: 158, a SV-40 poly(A) signal sequence exemplified by SEQ ID NO: 143, optionally a cloning site exemplified by SEQ ID NO: 164, a U6 promoter sequence exemplified by SEQ ID NO: 144, a guide RNA sequence exemplified by SEQ ID NO: 47, a tcrRNA sequence exemplified by SEQ ID NO: 147, a Pol III transcription termination sequence exemplified by SEQ ID NO: 149, optionally a cloning site exemplified by SEQ ID NO: 170, and a 3′ ITR exemplified by SEQ ID NO: 20.

Three (3) eGFP and sgRNA Construct Sequences

In one embodiment, an exemplary construct comprises: a 5′ ITR exemplified by SEQ ID NO: 19, optionally a cloning site exemplified by SEQ ID NO: 160, a CMV enhancer exemplified by SEQ ID NO: 98, a CMV promoter exemplified by SEQ ID NO: 100, optionally a cloning site exemplified by SEQ ID NO: 161, a turboGFP coding sequence exemplified by SEQ ID NO: 142, optionally a cloning site exemplified by SEQ ID NO: 166, a bGHpoly(a) signal exemplified by SEQ ID NO: 36, optionally a cloning site exemplified by SEQ ID NO: 164, a U6 promoter sequence exemplified by SEQ ID NO: 144, a guide RNA sequence exemplified by SEQ ID NO: 42, a tcrRNA sequence exemplified by SEQ ID NO: 147, a Pol III transcription termination sequence exemplified by SEQ ID NO: 149, optionally a cloning site exemplified by SEQ ID NO: 167, and a 3′ ITR exemplified by SEQ ID NO: 20

In one embodiment, an exemplary construct comprises: a 5′ ITR exemplified by SEQ ID NO: 19, optionally a cloning site exemplified by SEQ ID NO: 160, a CMV enhancer exemplified by SEQ ID NO: 98, a CMV promoter exemplified by SEQ ID NO: 100, optionally a cloning site exemplified by SEQ ID NO: 161, a turboGFP coding sequence exemplified by SEQ ID NO: 142, optionally a cloning site exemplified by SEQ ID NO: 166, a bGHpoly(a) signal exemplified by SEQ ID NO: 36, optionally a cloning site exemplified by SEQ ID NO: 164, a U6 promoter sequence exemplified by SEQ ID NO: 144, a guide RNA sequence exemplified by SEQ ID NO: 43, a tcrRNA sequence exemplified by SEQ ID NO: 147, a Pol III transcription termination sequence exemplified by SEQ ID NO: 149, optionally a cloning site exemplified by SEQ ID NO: 167, and a 3′ ITR exemplified by SEQ ID NO: 20

In one embodiment, an exemplary construct comprises: a 5′ ITR exemplified by SEQ ID NO: 19, optionally a cloning site exemplified by SEQ ID NO: 160, a CMV enhancer exemplified by SEQ ID NO: 98, a CMV promoter exemplified by SEQ ID NO: 100, optionally a cloning site exemplified by SEQ ID NO: 161, a turboGFP coding sequence exemplified by SEQ ID NO: 142, optionally a cloning site exemplified by SEQ ID NO: 166, a bGHpoly(a) signal exemplified by SEQ ID NO: 36, optionally a cloning site exemplified by SEQ ID NO: 164, a U6 promoter sequence exemplified by SEQ ID NO: 144, a guide RNA sequence exemplified by SEQ ID NO: 150, a tcrRNA sequence exemplified by SEQ ID NO: 147, a Pol III transcription termination sequence exemplified by SEQ ID NO: 149, optionally a cloning site exemplified by SEQ ID NO: 167, and a 3′ ITR exemplified by SEQ ID NO: 20.

Ten (10) miRNA Construct Sequences Comprising Mouse KCNQ4 Targeting Sequences

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a human microRNA backbone and mouse KCNQ4 targeting sequence exemplified by SEQ ID NO: 135 engineered into the chimeric intron, optionally a cloning site exemplified by SEQ ID NO: 156, an eGFP coding region exemplified by SEQ ID NO: 103, optionally a cloning site exemplified by SEQ ID NO: 157, a microRNA backbone and KCNQ4 targeting sequence exemplified by SEQ ID NO: 135, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a human microRNA backbone and mouse KCNQ4 targeting sequence exemplified by SEQ ID NO: 135 engineered into the chimeric intron, optionally a cloning site exemplified by SEQ ID NO: 156, an KCNQ4 coding region exemplified by SEQ ID NO: 10, optionally a cloning site exemplified by SEQ ID NO: 157, a microRNA backbone and KCNQ4 targeting sequence exemplified by SEQ ID NO: 135, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a human microRNA backbone and mouse KCNQ4 targeting sequence exemplified by SEQ ID NO: 136 engineered into the chimeric intron, optionally a cloning site exemplified by SEQ ID NO: 156, an eGFP coding region exemplified by SEQ ID NO: 103, optionally a cloning site exemplified by SEQ ID NO: 157, a microRNA backbone and KCNQ4 targeting sequence exemplified by SEQ ID NO: 135, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a human microRNA backbone and mouse KCNQ4 targeting sequence exemplified by SEQ ID NO: 136 engineered into the chimeric intron, optionally a cloning site exemplified by SEQ ID NO: 156, an KCNQ4 coding region exemplified by SEQ ID NO: 10, optionally a cloning site exemplified by SEQ ID NO: 157, a microRNA backbone and KCNQ4 targeting sequence exemplified by SEQ ID NO: 135, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a human microRNA backbone and mouse KCNQ4 targeting sequence exemplified by SEQ ID NO: 137 engineered into the chimeric intron, optionally a cloning site exemplified by SEQ ID NO: 156, an eGFP coding region exemplified by SEQ ID NO: 103, optionally a cloning site exemplified by SEQ ID NO: 157, a microRNA backbone and KCNQ4 targeting sequence exemplified by SEQ ID NO: 135, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a human microRNA backbone and mouse KCNQ4 targeting sequence exemplified by SEQ ID NO: 137 engineered into the chimeric intron, optionally a cloning site exemplified by SEQ ID NO: 156, an KCNQ4 coding region exemplified by SEQ ID NO: 10, optionally a cloning site exemplified by SEQ ID NO: 157, a microRNA backbone and KCNQ4 targeting sequence exemplified by SEQ ID NO: 135, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a human microRNA backbone and mouse KCNQ4 targeting sequence exemplified by SEQ ID NO: 136 engineered into the chimeric intron, optionally a cloning site exemplified by SEQ ID NO: 156, an eGFP coding region exemplified by SEQ ID NO: 103, optionally a cloning site exemplified by SEQ ID NO: 157, three copies of microRNA backbone and KCNQ4 targeting sequence exemplified by SEQ ID NO: 138, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 18.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with three copies of human microRNA backbone and mouse KCNQ4 targeting sequence exemplified by SEQ ID NO: 139 engineered into the chimeric intron, optionally a cloning site exemplified by SEQ ID NO: 156, an eGFP coding region exemplified by SEQ ID NO: 103, optionally a cloning site exemplified by SEQ ID NO: 157, three copies of microRNA backbone and KCNQ4 targeting sequence exemplified by SEQ ID NO: 138, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 18.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with a human microRNA backbone and mouse KCNQ4 targeting sequence exemplified by SEQ ID NO: 136 engineered into the chimeric intron, optionally a cloning site exemplified by SEQ ID NO: 156, an KCNQ4 coding region exemplified by SEQ ID NO: 10, optionally a cloning site exemplified by SEQ ID NO: 157, three copies of microRNA backbone and KCNQ4 targeting sequence exemplified by SEQ ID NO: 138, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 18.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a CBA promoter exemplified by SEQ ID NO: 101, a chimeric intron exemplified by SEQ ID NO: 102 with three copies of human microRNA backbone and mouse KCNQ4 targeting sequence exemplified by SEQ ID NO: 139 engineered into the chimeric intron, optionally a cloning site exemplified by SEQ ID NO: 156, an KCNQ4 coding region exemplified by SEQ ID NO: 10, optionally a cloning site exemplified by SEQ ID NO: 157, three copies of microRNA backbone and KCNQ4 targeting sequence exemplified by SEQ ID NO: 138, optionally a cloning site exemplified by SEQ ID NO: 158, a polyA site exemplified by SEQ ID NO: 36, optionally a cloning site exemplified by SEQ ID NO: 159, and a 3′ ITR exemplified by SEQ ID NO: 18.

Two (2) Codon Optimized KCNQ4 with sgRNA Construct Sequences

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a chicken B-actin promoter exemplified by SEQ ID NO: 101, a chimeric intron exemplified by SEQ ID NO: 24, optionally a cloning site exemplified by SEQ ID NO: 156, an KCNQ4 coding region exemplified by SEQ ID NO: 9, optionally a cloning site exemplified by SEQ ID NO: 221, a bGH poly(A) signal exemplified by SEQ ID NO: 36, a U6 promoter sequence exemplified by SEQ ID NO: 144, a guide RNA sequence exemplified by SEQ ID NO: 42, a tracrRNA sequence exemplified by SEQ ID NO: 147, a Pol III transcription termination sequence exemplified by SEQ ID NO: 149, and a 3′ ITR exemplified by SEQ ID NO: 20.

In one embodiment, an exemplary construct comprises: a 5′ ITR exemplified by SEQ ID NO: 19, optionally a cloning site exemplified by SEQ ID NO: 155, a CMV enhancer exemplified by SEQ ID NO: 22, a chicken B-actin promoter exemplified by SEQ ID NO: 101, a chimeric intron exemplified by SEQ ID NO: 24, optionally a cloning site exemplified by SEQ ID NO: 156, an KCNQ4 coding region exemplified by SEQ ID NO: 9, optionally a cloning site exemplified by SEQ ID NO: 221, a bGH poly(A) signal coding region exemplified by SEQ ID NO: 36, a U6 promoter sequence exemplified by SEQ ID NO: 144, a guide RNA sequence exemplified by SEQ ID NO: 150, a tracrRNA sequence exemplified by SEQ ID NO: 147, a Pol III transcription termination sequence exemplified by SEQ ID NO: 149, and a 3′ ITR exemplified by SEQ ID NO: 20.

Exemplary Construct Component Sequences

Exemplary AAV 5′ ITR sequence
(SEQ ID NO: 19)
CTGCGCGCTCGCTCGCTCACTGAGGCCGCCCGGGCGTCGGGCGACCTTTGGTCGCCCGGCCTCA
GTGAGCGAGCGAGCGCGCAGAGAGGGAGTGGCCAACTCCATCACTAGGGGTTCCT
Exemplary AAV 3′ ITR sequence
(SEQ ID NO: 20)
AGGAACCCCTAGTGATGGAGTTGGCCACTCCCTCTCTGCGCGCTCGCTCGCTCACTGAGGCCGG
GCGACCAAAGGTCGCCCGACGCCCGGGCTTTGCCCGGGCGGCCTCAGTGAGCGAGCGAGCGCGC
AG
Exemplary CMV Enhancer sequence
(SEQ ID NO: 22)
GAGATTGATTATTGAGTAGTTATTAATAGTAATCAATTACGGGGTCATTAGTTCATAGCCCATA
TATGGAGTTCCGCGTTACATAACTTACGGTAAATGGCCCGCCTGGCTGACCGCCCAACGACCCC
CGCCCATTGAGGTCAATAATGAGGTATGTTCCCATAGTAACGCCAATAGGGAGTTTCCATTGAG
GTCAATGGGTGGACTATTTACGGTAAACTGCCCACTTGGCAGTACATCAAGTGTATCATATGCC
AAGTACGCCCCCTATTGACGTCAATGACGGTAAATGGCCCGCCTGGCATTATGCCCAGTACATG
ACCTTATGGGACTTTCCTACTTGGCAGTACATCTACGTATTAGTCATCGCTATTACCATGG
Exemplary CMV Enhancer sequence
(SEQ ID NO: 98)
CGTTACATAACTTACGGTAAATGGCCCGCCTGGCTGACCGCCCAACGACCCCCGCCCATTGACG
TCAATAATGACGTATGTTCCCATAGTAACGCCAATAGGGACTTTCCATTGACGTCAATGGGTGG
AGTATTTACGGTAAACTGCCCACTTGGCAGTACATCAAGTGTATCATATGCCAAGTACGCCCCC
TATTGACGTCAATGACGGTAAATGGCCCGCCTGGCATTATGCCCAGTACATGACCTTATGGGAC
TTTCCTACTTGGGAGTAGATCTAGGTATTAGTCATCGCTATTAGCATG
Exemplary CMV Promoter sequence
(SEQ ID NO: 99)
GTGATGCGGTTTTGGCAGTACATCAATGGGCGTGGATAGCGGTTTGACTCACGGGGATTTCCAA
GTCTCCACCCCATTGACGTCAATGGGAGTTTGTTTTGGCACCAAAATCAACGGGACTTTCCAAA
ATGTCGTAACAACTCCGCCCCATTGACGCAAATGGGCGGTAGGCGTGTACGGTGGGAGGTCTAT
ATAAGCAGAGCTCGTTTAGTGAACCGT
Exemplary CMV Promoter sequence
(SEQ ID NO: 100)
GTGATGCGGTTTTGGCAGTACATCAATGGGCGTGGATAGCGGTTTGACTCACGGGGATTTCCAA
GTCTCCACCCCATTGACGTCAATGGGAGTTTGTTTTGGCACCAAAATCAACGGGACTTTCCAAA
ATGTCGTAACAACTCCGCCCCATTGACGCAAATGGGCGGTAGGCGTGTACGGTGGGAGGTCTAT
ATAAGCAGAGCTCGTTTAGTGAACCGTCGTTTAGTGAACCGT
Exemplary Chicken B-actin promoter sequence
(SEQ ID NO: 101)
GTCGAGGTGAGCCCCACGTTCTGCTTCACTCTCCCCATCTCCCCCCCCTCCCCACCCCCAATTT
TGTATTTATTTATTTTTTAATTATTTTGTGCAGCGATGGGGGCGGGGGGGGGGGGGGCGCGCGC
CAGGCGGGGCGGGGCGGGGCGAGGGGCGGGGCGGGGCGAGGCGGAGAGGTGCGGCGGCAGCCAA
TCAGAGCGGCGCGCTCCGAAAGTTTCCTTTTATGGCGAGGCGGCGGCGGCGGCGGCCCTATAAA
AAGCGAAGCGCGCGGCGGGCG
Exemplary Chicken B-actin promoter sequence
(SEQ ID NO: 23)
GTCGAGGTGAGCCCCACGTTCTGCTTCACTCTCCCCATCTCCCCCCCCTCCCCACCCCCAATTT
TGTATTTATTTATTTTTTAATTATTTTGTGCAGCGATGGGGGCGGGGGGGGGGGGGGGGCGCGC
GCCAGGCGGGGCGGGGCGGGGCGAGGGGCGGGGCGGGGCGAGGCGGAGAGGTGCGGCGGCAGCC
AATCAGAGCGGCGCGCTCCGAAAGTTTCCTTTTATGGCGAGGCGGCGGCGGCGGCGGCCCTATA
AAAAGCGAAGCGCGCGGCGGGCG
Exemplary SV-40 chimeric intron sequence
(SEQ ID NO: 102)
GGAGTCGCTGCGTTGCCTTCGCCCCGTGCCCCGCTCCGCGCCGCCTCGCGCCGCCCGCCCCGGC
TCTGACTGACCGCGTTACTCCCACAGGTGAGCGGGCGGGACGGCCCTTCTCCTCCGGGCTGTAA
TTAGCGCTTGGTTTAATGACGGCTCGTTTCTTTTCTGTGGCTGCGTGAAAGCCTTAAAGGGCTC
CGGGAGGGCCCTTTGTGCGGGGGGGAGCGGCTCGGGGGGTGCGTGCGTGTGTGTGTGCGTGGGG
AGCGCCGCGTGCGGCCCGCGCTGCCCGGCGGCTGTGAGCGCTGCGGGCGCGGCGCGGGGCTTTG
TGCGCTCCGCGTGTGCGCGAGGGGAGCGCGGCCGGGGGCGGTGCCCCGCGGTGCGGGGGGGCTG
CGAGGGGAACAAAGGCTGCGTGCGGGGTGTGTGCGTGGGGGGGTGAGCAGGGGGTGTGGGCGCG
GCGGTCGGGCTGTAACCCCCCCCTGCACCCCCCTCCCCGAGTTGCTGAGCACGGCCCGGCTTCG
GGTGCGGGGCTCCGTGCGGGGCGTGGCGCGGGGCTCGCCGTGCCGGGCGGGGGGTGGCGGCAGG
TGGGGGTGCCGGGCGGGGCGGGGCCGCCTCGGGCCGGGGAGGGCTCGGGGGAGGGGCGCGGCGG
CCCCCGGAGCGCCGGCGTCTAGAGCCTCTGCTAACCATGTTCATGCCTTCTTCTTTTTCCTACA
G
Exemplary SV-40 chimeric intron sequence
(SEQ ID NO: 24)
GGAGTCGCTGCGTTGCCTTCGCCCCGTGCCCCGCTCCGCGCCGCCTCGCGCCGCCCGCCCCGGC
TCTGACTGACCGCGTTACTCCCACAGGTGAGCGGGCGGGACGGCCCTTCTCCTCCGGGCTGTAA
TTAGCGCTTGGTTTAATGACGGCTCGTTTCTTTTCTGTGGCTGCGTGAAAGCCTTAAAGGGCTC
CGGGAGGGCCCTTTGTGCGGGGGGGAGCGGCTCGGGGGGTGCGTGCGTGTGTGTGTGCGTGGGG
AGCGCCGCGTGCGGCCCGCGCTGCCCGGCGGCTGTGAGCGCTGCGGGCGCGGCGCGGGGCTTTG
TGCGCTCCGCGTGTGCGCGAGGGGAGCGCGGCCGGGGGCGGTGCCCCGCGGTGCGGGGGGGCTG
CGAGGGGAACAAAGGCTGCGTGCGGGGTGTGTGCGTGGGGGGGTGAGCAGGGGGTGTGGGCGCG
GCGGTCGGGCTGTAACCCCCCCCTGCACCCCCCTCCCCGAGTTGCTGAGCACGGCCCGGCTTCG
GGTGCGGGGCTCCGTGCGGGGCGTGGCGCGGGGCTCGCCGTGCCGGGCGGGGGGTGGCGGCAGG
TGGGGGTGCCGGGCGGGGCGGGGCCGCCTCGGGCCGGGGAGGGCTCGGGGGAGGGGCGCGGCGG
CCCCCGGAGCGCCGGCGGCTGTCGAGGCGCGGCGAGCCGCAGCCATTGCCTTTTATGGTAATCG
TGCGAGAGGGCGCAGGGACTTCCTTTGTCCCAAATCTGTGCGGAGCCGAAATCTGGGAGGCGCC
GCCGCACCCCCTCTAGCGGGCGCGGGGCGAAGCGGTGCGGCGCCGGCAGGAAGGAAATGGGCGG
GGAGGGCCTTCGTGCGTCGCCGCGCCGCCGTCCCCTTCTCCCTCTCCAGCCTCGGGGCTGTCCG
CGGGGGGACGGCTGCCTTCGGGGGGGACGGGGCAGGGCGGGGTTCGGCTTCTGGCGTGTGACCG
GCGGCTCTAGAGCCTCTGCTAACCATGTTCATGCCTTCTTCTTTTTCCTACAG
Exemplary EGFP sequence
(SEQ ID NO: 103)
ATGGTGAGCAAGGGCGAGGAGCTGTTCACCGGGGTGGTGCCCATCCTGGTCGAGCTGGACGGCG
ACGTAAACGGCCACAAGTTCAGCGTGTCCGGCGAGGGCGAGGGCGATGCCACCTACGGCAAGCT
GACCCTGAAGTTCATCTGCACCACCGGCAAGCTGCCCGTGCCCTGGCCCACCCTCGTGACCACC
CTGACCTACGGCGTGCAGTGCTTCAGCCGCTACCCCGACCACATGAAGCAGCACGACTTCTTCA
AGTCCGCCATGCCCGAAGGCTACGTCCAGGAGCGCACCATCTTCTTCAAGGACGACGGCAACTA
CAAGACCCGCGCCGAGGTGAAGTTCGAGGGCGACACCCTGGTGAACCGCATCGAGCTGAAGGGC
ATCGACTTCAAGGAGGACGGCAACATCCTGGGGCACAAGCTGGAGTACAACTACAACAGCCACA
ACGTCTATATCATGGCCGACAAGCAGAAGAACGGCATCAAGGTGAACTTCAAGATCCGCCACAA
CATCGAGGACGGCAGCGTGCAGCTCGCCGACCACTACCAGCAGAACACCCCCATCGGCGACGGC
CCCGTGCTGCTGCCCGACAACCACTACCTGAGCACCCAGTCCGCCCTGAGCAAAGACCCCAACG
AGAAGCGCGATCACATGGTCCTGCTGGAGTTCGTGACCGCCGCCGGGATCACTCTCGGCATGGA
CGAGCTGTACAAGTAA
Exemplary bGH poly(A) signal sequence
(SEQ ID NO: 36)
CTGTGCCTTCTAGTTGCCAGCCATCTGTTGTTTGCCCCTCCCCCGTGCCTTCCTTGACCCTGGA
AGGTGCCACTCCCACTGTCCTTTCCTAATAAAATGAGGAAATTGCATCGCATTGTCTGAGTAGG
TGTCATTCTATTCTGGGGGGTGGGGTGGGGCAGGACAGCAAGGGGGAGGATTGGGAAGACAATA
GCAGGCATGCTGGGGATGCGGTGGGCTCTATGG
Exemplary microRNA-16 backbone with KCNQ4 targeting
seq #2 sequence
(SEQ ID NO: 104)
CATAGCTCTTATGATAGCAATGTCAGCAGTGCCTTTGAGTCTCAGAGATGCCCGGGTTAAGATT
CTAAAATTATCACGGGTATTACTTGAGGCTCAGAAGTAAGGTTGACCATACTCTACAGTTGTG
Exemplary microRNA-26 backbone with KCNQ4 targeting
seq #2 sequence
(SEQ ID NO: 105)
GAGCAGGAGGACCAAGGCCCTGGCGAAGGCCGTGGCCTCGTTGAGTCTCAGAGATGCCCGGTGT
GCAGGTCCCAATGGCCGGGTATTACTGAGATTCCACGGGGACGCGGGCCTGGACGCCGGCATCC
GGGCTCAGGACCCCCCTC
Exemplary microRNA-96 backbone with KCNQ4 targeting
seq #2 sequence
(SEQ ID NO: 106)
GTGTCCCCAGAGAGCCCGCACCAGTGCCATCTGCTTGGCCGATTTGAGTCTCAGAGATGCCCgg
CTTGTGTCTCTCCGCTCTGAGCTGTCTGTCTCGAGGCTCATATGGGAAAAGCAGGACCCGCAGC
TGCGTCCGCCTCCCCTGCAT
Exemplary miRNA-16 backbone with KCNQ4 targeting
seq #4 sequence
(SEQ ID NO: 107)
CATAGCTCTTATGATAGCAATGTCAGCAGTGCCTTTCTCGAAGTGCTTCTGCCGGGTTAAGATT
CTAAAATTATCACGGTAGAAACATCTTCGGGAGAAGTAAGGTTGAGCATACTCTAGAGTTGTG
Exemplary miRNA-26 backbone with KCNQ4 targeting
seq #4 sequence
(SEQ ID NO: 108)
GAGCAGGAGGACCAAGGCCCTGGCGAAGGCCGTGGCCTCGTTCTCGAAGTGCTTCTGCCGGTGT
GCAGGTCCCAATGGCCGGTAGAATCGTTTCGAGCACGGGGACGCGGGCCTGGACGCCGGCATCC
GGGCTCAGGACCCCCCTC
Exemplary miRNA-96 backbone with KCNQ4 targeting
seq #4 sequence
(SEQ ID NO: 109)
GTGTCCCCAGAGAGCCCGCACCAGTGCCATCTGCTTGGCCGATTTCTCGAAGTGCTTCTGCCGG
CTTGTGTCTCTCCGCTCTGAGCTGTGAGGAGCTTTCGGGATATGGGAAAAGCAGGACCCGCAGC
TGCGTCCGCCTCCCCTGCAT
Exemplary miRNA-16 backbone with KCNQ4 targeting
seq #5 sequence
(SEQ ID NO: 110)
CATAGCTCTTATGATAGCAATGTCAGCAGTGCCTTTCTCCACCTTGACCACGCGTGTTAAGATT
CTAAAATTATCTCGTGTGGTAAGTGGTGGAGAGAAGTAAGGTTGACCATACTCTACAGTTGTG
Exemplary miRNA-26 backbone with KCNQ4 targeting
seq #5 sequence
(SEQ ID NO: ill)
GAGCAGGAGGACCAAGGCCCTGGCGAAGGCCGTGGCCTCGTTCTCCACCTTGACCACGCGTTGT
GCAGGTCCCAATGGGCGTGTGGTAGAGGTGGAGCACGGGGACGCGGGCCTGGACGCCGGCATCC
GGGCTCAGGACCCCCCTC
Exemplary miRNA-96 backbone with KCNQ4 targeting
seq #5 sequence
(SEQ ID NO: 112)
GTGTCCCCAGAGAGCCCGCACCAGTGCCATCTGCTTGGCCGATTTCTCCACCTTGACCACGCGT
CTTGTGTCTCTCCGCTCTGAGGTGACTGGTTGGGTGGAGATATGGGAAAAGCAGGACCCGCAGC
TGCGTCCGCCTCCCCTGCAT
Exemplary miRNA-16 backbone with KCNQ4 targeting
seq #6 sequence
(SEQ ID NO: 113)
CATAGCTCTTATGATAGCAATGTCAGCAGTGCCTATACTGTCATAGTAGTACCAGGTTAAGATT
CTAAAATTATCATGGTACTAATGTTGGGAGTGTAAGTAAGGTTGAGCATACTCTAGAGTTGTG
Exemplary miRNA-26 backbone with KCNQ4 targeting
seq #6 sequence
(SEQ ID NO: 114)
GAGCAGGAGGACCAAGGCCCTGGCGAAGGCCGTGGCCTCGATACTGTCATAGTAGTACCAGTGT
GCAGGTCCCAATGGCTGGTACTAATGTGACAGTCTCGGGGACGCGGGCCTGGACGCCGGCATCC
GGGCTCAGGACCCCCCTC
Exemplary miRNA-96 backbone with KCNQ4 targeting
seq #6 sequence
(SEQ ID NO: 115)
GTGTCCCCAGAGAGCCCGCACCAGTGCCATCTGCTTGGCCGATATACTGTCATAGTAGTACCAG
CTTGTGTCTCTCCGCTCTGAGCTGTCACTGCTTGACGGTAAATGGGAAAAGCAGGACCCGCAGC
TGCGTCCGCCTCCCCTGCAT
Exemplary miRNA-155 backbone with KCNQ4 targeting
sequence #1 sequence
(SEQ ID NO: 116)
TCCTCTGAGTGCTGAAGGCTTGCTGTAGGCTGTATGCTGTTGAGAATCCTGATGGAGCGGGTTT
TTGCCTCCAACTGACCCGCTCATAGGATTCTCAACAGTGTATGATGCCTGTTACTAGCATTCAC
ATGGA
Exemplary miRNA-155 backbone with KCNQ4 targeting
sequence #2 sequence
(SEQ ID NO: 117)
TCCTCTGAGTGCTGAAGGCTTGCTGTAGGCTGTATGCTGTTGAGTCTCAGAGATGCCCGGGTTT
TTGCCTCCAACTGACTCGGGATTCTGAGACTCAACAGTGTATGATGCCTGTTACTAGCATTCAC
ATGGA
Exemplary miRNA-155 backbone with KCNQ4 targeting
sequence #2v2 sequence
(SEQ ID NO: 118)
TCCTCTGAGTGCTGAAGGCTTGCTGTAGGCTGTATGCTGTTGAGTCTCAGAGATGCCCGGGTTT
TTGCCTCCAACTGACTCGGGATTCTGAGATTCAACAGTGTATGATGCCTGTTACTAGCATTCAC
ATGGA
Exemplary miRNA-155 backbone with KCNQ4 targeting
sequence #2v3 sequence
(SEQ ID NO: 119)
TCCTCTGAGTGCTGAAGGCTTGCTGTAGGCTGTATGCTGTTGAGTCTCAGAGATGCCCGGGTTT
TTGCCTCCAACTGACTCGGGATCCTGAGATTCAACAGTGTATGATGCCTGTTACTAGCATTCAC
ATGGA
Exemplary miRNA-155 backbone with KCNQ4 targeting
sequence #2v4 sequence
(SEQ ID NO: 120)
TCCTCTGAGTGCTGAAGGCTTGCTGTAGGCTGTATGCTGTTGAGTCTCAGAGATGCCCGGAAGT
TTGCCTCCAACTCTTTCGGGATCCTGAGATTCAACAGTGTATGATGCCTGTTACTAGCATTCAC
ATGGA
Exemplary miRNA-155 backbone with KCNQ4 targeting
sequence #4 sequence
(SEQ ID NO: 121)
TCCTCTGAGTGCTGAAGGCTTGCTGTAGGCTGTATGCTGTTCTCGAAGTGCTTCTGCCGGGTTT
TTGCCTCCAACTGACTCGGCGAATACTTCGAGAACAGTGTATGATGCCTGTTACTAGCATTCAC
ATGGA
Exemplary miRNA-155 backbone with KCNQ4 targeting
sequence #5 sequence
(SEQ ID NO: 122)
TCCTCTGAGTGCTGAAGGCTTGCTGTAGGCTGTATGCTGTTCTCCACCTTGACCACGCGTGTTT
TTGCCTCCAACTGACATGTGGGTAAGGTGGAGAACAGTGTATGATGCCTGTTACTAGCATTCAC
ATGGA
Exemplary miRNA-155 backbone with KCNQ4 targeting
sequence #6 sequence
(SEQ ID NO: 123)
TCCTCTGAGTGCTGAAGGCTTGCTGTAGGCTGTATGCTGATACTGTCATAGTAGTACCAGGTTT
TTGCCTCCAACTGACCTGGTCTATATGACAGTATCAGTGTATGATGCCTGTTACTAGCATTCAC
ATGGA
Exemplary miRNA-155 backbone with KCNQ4 targeting
sequence #7 sequence
(SEQ ID NO: 124)
TCCTCTGAGTGCTGAAGGCTTGCTGTAGGCTGTATGCTGTAGTCGGAGGTGATGTCGGGGGTTT
TTGCCTCCAACTGACTCCTGCATACCTTCGATTACAGTGTATGATGCCTGTTACTAGCATTCAC
ATGGA
Exemplary miRNA-16 backbone with KCNQ4 targeting
sequence #1 sequence
(SEQ ID NO: 125)
CATAGCTCTTATGATAGCAATGTCAGCAGTGCCTTTGAGAATCCTGATGGAGCGGGTTAAGATT
CTAAAATTATCACGCTCCATGAGTGATTCTCAGAAGTAAGGTTGAGCATACTCTAGAGTTGTG
Exemplary miRNA-26 backbone with KCNQ4 targeting
sequence #1 sequence
(SEQ ID NO: 126)
GAGCAGGAGGACCAAGGCCCTGGCGAAGGCCGTGGCCTCGTTGAGAATCCTGATGGAGCGGTGT
GCAGGTCCCAATGGCCGCTCCATAAGGATTCTCCACGGGGACGCGGGCCTGGACGCCGGCATCC
GGGCTCAGGACCCCCCTC
Exemplary miRNA-96 backbone with KCNQ4 targeting
sequence #1 sequence
(SEQ ID NO: 127)
GTGTCCCCAGAGAGCCCGCACCAGTGCCATCTGCTTGGCCGATTTGAGAATCCTGATGGAGCGG
CTTGTGTCTCTCCGCTCTGAGCCGGGCCATCAGATTCTCATATGGGAAAAGCAGGACCCGCAGC
TGCGTCCGCCTCCCCTGCAT
Exemplary miRNA-122 backbone with KCNQ4 targeting
sequence #1 sequence
(SEQ ID NO: 128)
TGAAGTTAACACCTTCGTGGCTACAGAGTTTCCTTAGCAGAGCTGTTGAGAATCCTGATGGAGC
ggGTGTCTAAACTATCccGCTCCATCcGGATTCTACATAGCTACTGCTAGGCAATCCTTCCCTC
GATAAATGTCTTGGCATCG
Exemplary miRNA-135 backbone with KCNQ4 targeting
sequence #1 sequence
(SEQ ID NO: 129)
GCCGCTGGACCCCTCCACTCTGCTGTGGCCTTGAGAATCCTGATGGAGCGGGATTGCTGTCCCA
AACTCCCGCTCCATCGGATTCTCAAGGCTACAGTGAGGGGCGAGCTCCTTCTCCTGC
Exemplary miRNA-182 backbone with KCNQ4 targeting
sequence #1 sequence
(SEQ ID NO: 130)
CTCCCCAGCTCCTGGGGGGAGCTGCTTGCCTCCCCCCGTTTTGAGAATCCTGATGGAGCGGACT
GGTGAGGTAACAGGATCCGCCGTCCATCTTTATTCTCACTATGGGGCGAGGACTCAGCCGGCAC
CCTGTGCACAGCCAGCGAGGG
Exemplary miRNA-183 backbone with KCNQ4 targeting
sequence #1 sequence
(SEQ ID NO: 131)
AGGGTCGGCAGGCCGCAGAGTGTGACTCCTGTTCTGTGTTTGAGAATCCTGATGGAGCGGGTGA
ACAGTCTCAGTCCCGCTCCCAGGAAATGGTCAAAAACAGAGCAGAGACAGATCCACGAGGGCCT
CCGGAGCACCTT
Exemplary miRNA-335 backbone with KCNQ4 targeting
sequence #1 sequence
(SEQ ID NO: 132)
GAAACAGATTGGAAATGATTTGTTTTGAGCGGGGGTTGAGAATCCTGATGGAGCGGGTTTGTCA
TAAACCCCGCTCCCTCAGGATTCCCAACCTCCTCTCATTTGCTATATTCAATTAAGTAA
Exemplary miRNA-451 backbone with KCNQ4 targeting
sequence #1 sequence
(SEQ ID NO: 133)
TGACTGCCAGGGCACTTGGGAATGGCAAGGTTGAGAATCCTGATGGAGCGGATTCCATCAGGAT
TCTCAGTCTTGCTATACCCAGAAAACGTGCCAGGAAGA
Exemplary miRNA-155 backbone with KCNQ4 targeting
sequence #lv2 sequence
(SEQ ID NO: 134)
CCTGCAGGCGCCGGCGTCCTCTGAGTGCTGAAGGCTTGCTGTAGGCTGTATGCTGTTGAGAATC
CTGATGGAGCGGGTTTTTGCCTCCAACTGACCCGCTCATAGGATTCTCAACAGTGTATGATGCC
TGTTAGTAGCATTCACATGGA
Exemplary miRNA-26 backbone with mouse KCNQ4 targeting
sequence #6 sequence
(SEQ ID NO: 135)
GAGCAGGAGGACCAAGGCCCTGGCGAAGGCCGTGGCCTCGATGCTGTCATAGTAATACCAGTGT
GCAGGTCCCAATGGCTGGTATTAATGTGACAGTCTCGGGGACGCGGGCCTGGACGCCGGCATCC
GGGCTCAGGACCCCCCTC
Exemplary microRNA-26 backbone with mouse KCNQ4 targeting
sequence #2 sequence
(SEQ ID NO: 136)
GAGCAGGAGGACCAAGGCCCTGGCGAAGGCCGTGGCCTCGTTGAGTCTTAGAGAGGCCCGGTGT
GCAGGTCCCAATGGCCGGGTCTTACTGAGATTCCACGGGGACGCGGGCCTGGACGCCGGCATCC
GGGCTCAGGACCCCCCTC
Exemplary miRNA-26 backbone with mouse KCNQ4 targeting
sequence #5 sequence
(SEQ ID NO: 137)
GAGCAGGAGGACCAAGGCCCTGGCGAAGGCCGTGGCCTCGTTTTCCACCTTGACTACGCGCTGT
GCAGGTCCCAATGGGCGTGTAGTAGAGGTGGAGCACGGGGACGCGGGCCTGGACGCCGGCATCC
GGGCTCAGGACCCCCCTC
Exemplary three copies of miRNA-26 backbone with mouse
KCNQ4 targeting sequence #6
sequence
(SEQ ID NO: 138)
GTGGCCTCGATGCTGTCATAGTAATACCAGTGTGCAGGTCCCAATGGCTGGTATTAATGTGACA
GTCTCGGGGACGCGCAATTGGGTGGCCTCGATGCTGTCATAGTAATACCAGTGTGCAGGTCCCA
ATGGCTGGTATTAATGTGACAGTCTCGGGGACGCGGGCGCGCCCGTGGCCTCGATGCTGTCATA
GTAATACCAGTGTGCAGGTCCCAATGGCTGGTATTAATGTGACAGTCTCGGGGACGC
Exemplary three copies of miRNA-26 backbone with mouse
KCNQ4 targeting sequence #2
sequence
(SEQ ID NO: 139)
GTGGCCTCGTTGAGTCTTAGAGAGGCCCGGTGTGCAGGTCCCAATGGCCGGGTCTTACTGAGAT
TCCACGGGGACGCGGGATCCGGTGGCCTCGTTGAGTCTTAGAGAGGCCCGGTGTGCAGGTCCCA
ATGGCCGGGTCTTACTGAGATTCCACGGGGACGCGGGTACCCGTGGCCTCGTTGAGTCTTAGAG
AGGCCCGGTGTGCAGGTCCCAATGGCCGGGTCTTACTGAGATTCCACGGGGACGC
Exemplary U6shRNA-hKCNQ4-395 sequence
(SEQ ID NO: 48)
TCCTCATCTTGGAATTCGTGATGcgaaCATCACGAATTCCAAGATGAGGA
Exemplary U6shRNA-hKCNQ4-909 sequence
(SEQ ID NO: 49)
CGCCTTACTGGGCATCTCTTTCTcgaaAGAAAGAGATGCCCAGTAAGGCG
Exemplary U6shRNA-hKCNQ4-1095 sequence
(SEQ ID NO: 50)
CTGGTACTACTATGACAGTATcgaaATACTGTCATAGTAGTAGCAG
Exemplary U6shRNA-hKCNQ4-1531 sequence
(SEQ ID NO: 51)
AAGTAGCAGAGGAGAAGAGCTcgaaAGCTCTTCTCCTCTGCTACTT
Exemplary U6shRNA-hKCNQ4-1593 sequence
(SEQ ID NO: 52)
AAGACAGTCATCCGCTCCATCcgaaGATGGAGCGGATGACTGTCTT
Exemplary U6shRNA-hKCNQ4-1677 sequence
(SEQ ID NO: 53)
AAGGACGTCATTGAGCAGTACcgaaGTACTGCTCAATGACGTCCTT
Exemplary U6shRNA-hKCNQ4-1721 sequence
(SEQ ID NO: 54)
GCCGGATCAAGAGCCTGCAAAcgaaTTTGCAGGCTCTTGATCCGGC
Exemplary U6shRNA-hKCNQ4-395 sequence
(SEQ ID NO: 55)
GGTGGATGAAATCAGCATGATcgaaATCATGCTGATTTCATCCACC
Exemplary Luciferase sequence
(SEQ ID NO: 140)
ATGGAAGATGCCAAAAACATTAAGAAGGGCCCAGCGCCATTCTACCCACTCGAAGACGGGACCG
CCGGCGAGCAGCTGCACAAAGCCATGAAGCGCTACGCCCTGGTGCCCGGCACCATCGCCTTTAC
CGACGCACATATCGAGGTGGACATTACCTACGCCGAGTACTTCGAGATGAGCGTTCGGCTGGCA
GAAGCTATGAAGCGCTATGGGCTGAATACAAACCATCGGATCGTGGTGTGCAGCGAGAATAGCT
TGCAGTTCTTCATGCCCGTGTTGGGTGCCCTGTTCATCGGTGTGGCTGTGGCCCCAGCTAACGA
CATCTACAACGAGCGCGAGCTGCTGAACAGCATGGGCATCAGCCAGCCCACCGTCGTATTCGTG
AGCAAGAAAGGGCTGCAAAAGATCCTCAACGTGCAAAAGAAGCTACCGATCATACAAAAGATCA
TCATCATGGATAGCAAGACCGACTACCAGGGCTTCCAAAGCATGTACACCTTCGTGACTTCCCA
TTTGCCACCCGGCTTCAACGAGTACGACTTCGTGCCCGAGAGCTTCGACCGGGACAAAACCATC
GCCCTGATCATGAACAGTAGTGGCAGTACCGGATTGCCCAAGGGCGTAGCCCTACCGCACCGCA
CCGCTTGTGTCCGATTCAGTCATGCCCGCGACCCCATCTTCGGCAACCAGATCATCCCCGACAC
CGCTATCCTCAGCGTGGTGCCATTTCACCACGGCTTCGGCATGTTCACCACGCTGGGCTACTTG
ATCTGCGGCTTTCGGGTCGTGCTCATGTACCGCTTCGAGGAGGAGCTATTCTTGCGCAGCTTGC
AAGACTATAAGATTCAATCTGCCCTGCTGGTGCCCACACTATTTAGCTTCTTCGCTAAGAGCAC
TCTCATCGACAAGTACGACCTAAGCAACTTGCACGAGATCGCCAGCGGCGGGGCGCCGCTCAGC
AAGGAGGTAGGTGAGGCCGTGGCCAAACGCTTCCACCTACCAGGCATCCGCCAGGGCTACGGCC
TGACAGAAACAACCAGCGCCATTCTGATCACCCCCGAAGGGGACGACAAGCCTGGCGCAGTAGG
CAAGGTGGTGCCCTTCTTCGAGGCTAAGGTGGTGGACTTGGACACAGGTAAGACACTGGGTGTG
AACCAGCGCGGCGAGCTGTGCGTCCGTGGCCCCATGATCATGAGCGGCTACGTTAACAACCCCG
AGGCTACAAACGCTCTCATCGACAAGGACGGCTGGCTGCACAGCGGCGACATCGCCTACTGGGA
CGAGGACGAGCACTTCTTCATCGTGGACCGGCTGAAGAGCCTGATCAAATACAAGGGCTACCAG
GTAGCCCCAGCCGAACTGGAGAGCATCCTGCTGCAACACCCCAACATCTTCGACGCCGGGGTCG
CCGGCCTGCCCGACGACGATGCCGGCGAGCTGCCCGCCGCAGTCGTCGTGCTGGAACACGGTAA
AACCATGACCGAGAAGGAGATCGTGGACTATGTGGCCAGCCAGGTTACAACCGCCAAGAAGCTG
CGCGGTGGTGTTGTGTTCGTGGACGAGGTGCCTAAAGGACTGACCGGCAAGTTGGACGCCCGCA
AGATCCGCGAGATTCTCATTAAGGCCAAGAAGGGCGGCAAGATCGCCGTG
Exemplary T2A sequence
(SEQ ID NO: 141)
GAGGGCAGAGGAAGTCTGCTAACATGCGGTGACGTCGAGGAGAATCCTGGCCCA
Exemplary TurboGFP sequence
(SEQ ID NO: 142)
ATGGAGAGCGACGAGAGCGGCCTGCCCGCCATGGAGATCGAGTGCCGCATCACCGGCACCCTGA
ACGGCGTGGAGTTCGAGCTGGTGGGCGGCGGAGAGGGCACCCCCGAGCAGGGCCGCATGACCAA
CAAGATGAAGAGCACCAAAGGCGCCCTGACCTTCAGCCCCTACCTGCTGAGCCACGTGATGGGC
TACGGCTTCTACCACTTCGGCACCTACCCCAGCGGCTACGAGAACCCCTTCCTGCACGCCATCA
ACAACGGCGGCTACACCAACACCCGCATCGAGAAGTACGAGGACGGCGGCGTGCTGCACGTGAG
CTTCAGCTACCGCTACGAGGCCGGCCGCGTGATCGGCGACTTCAAGGTGATGGGCACCGGCTTC
CCCGAGGACAGCGTGATCTTCACCGACAAGATCATCCGCAGCAACGCCACCGTGGAGCACCTGC
ACCCCATGGGCGATAACGATCTGGATGGCAGCTTCACCCGCACCTTCAGCCTGCGCGACGGCGG
CTACTACAGCTCCGTGGTGGACAGCCACATGCACTTCAAGAGCGCCATCCACCCCAGCATCCTG
CAGAACGGGGGCCCCATGTTCGCCTTCCGCCGCGTGGAGGAGGATCACAGCAACACCGAGCTGG
GCATCGTGGAGTACCAGCACGCCTTCAAGACCCCGGATGCAGATGCCGGTGAAGAATAA
Exemplary SV40 poly(A) signal sequence
(SEQ ID NO: 143)
TAAGATACATTGATGAGTTTGGACAAACCACAACTAGAATGCAGTGAAAAAAATGCTTTATTTG
TGAAATTTGTGATGCTATTGCTTTATTTGTAACCATTATAAGCTGCAATAAACAAGTT
Exemplary U6 promoter sequence
(SEQ ID NO: 144)
AAGGTCGGGCAGGAAGAGGGCCTATTTCCCATGATTCCTTCATATTTGCATATACGATACAAGG
CTGTTAGAGAGATAATTAGAATTAATTTGACTGTAAACACAAAGATATTAGTACAAAATACGTG
ACGTAGAAAGTAATAATTTCTTGGGTAGTTTGCAGTTTTAAAATTATGTTTTAAAATGGACTAT
CATATGCTTACCGTAACTTGAAAGTATTTCGATTTCTTGGCTTTATATATCTTGTGGAAAGGAC
GAAACACCG
Exemplary hKCNQ4 codon optimized sequence
(SEQ ID NO: 9)
ATGGCTGAAGCCCCTCCTAGAAGGCTTGGACTGGGACCTCCTCCTGGGGATGCTCCTAGAGCTG
AACTGGTGGCTCTGACAGCCGTGCAGTCTGAACAAGGCGAAGCTGGTGGCGGCGGATCTCCACG
TAGACTTGGACTGCTGGGAAGCCCTCTTCCTCCTGGTGCTCCACTTCCTGGACCTGGCAGTGGA
TCTGGATCTGCCTGTGGCCAGAGAAGCTCTGCCGCTCACAAGAGATACCGGCGGCTGCAGAACT
GGGTGTACAACGTGCTGGAAAGACCCAGAGGCTGGGCCTTCGTGTACCACGTGTTCATCTTTCT
GCTGGTGTTCAGCTGCCTGGTGCTGTCCGTGCTGAGCACCATCCAAGAACATCAAGAGCTGGCT
AACGAGTGCCTGTTAATACTGGAGTTTGTGATGATTGTGGTGTTCGGCCTCGAGTACATCGTCC
GCGTTTGGAGCGCCGGCTGCTGCTGCAGATATAGAGGTTGGCAAGGCAGATTCCGCTTCGCCAG
AAAGCCCTTCTGCGTGATCGACTTCATCGTGTTCGTGGCCAGCGTGGCCGTGATTGCTGCTGGC
ACACAGGGCAACATCTTCGCCACAAGCGCCCTGCGGAGCATGCGGTTTCTGCAGATCCTGAGAA
TGGTCCGAATGGACAGAAGAGGCGGCACCTGGAAGCTGCTGGGCTCTGTGGTGTACGCCCACAG
CAAAGAGCTGATCACCGCCTGGTACATCGGATTTCTGGTGCTGATCTTCGCCTCCTTCCTGGTG
TACCTGGCCGAGAAGGACGCCAACAGCGACTTTAGCAGCTACGCCGACTCTCTTTGGTGGGGCA
CCATCACACTGACCACCATCGGCTACGGCGACAAGACCCCTCACACATGGCTGGGAAGAGTGCT
GGCCGCTGGATTTGCTCTGCTGGGCATCAGCTTTTTCGCCCTGCCTGCCGGAATCCTCGGATCT
GGCTTTGCCCTGAAGGTGCAAGAGCAGCACCGGCAGAAGCACTTCGAGAAGAGAAGAATGCCTG
CCGCCAACCTGATTCAGGCCGCTTGGAGACTGTACAGCACCGACATGAGCAGAGCCTACCTGAC
CGCCACGTGGTATTATTACGACTCGATCCTGCCTAGCTTCCGCGAACTGGCCCTGCTGTTTGAG
CATGTGCAGAGAGCCAGAAACGGCGGCCTCAGACCTCTGGAAGTTCGGAGAGCACCTGTGCCTG
ATGGCGCCCCTTCTAGATATCCTCCAGTGGCCACCTGTCACAGACCCGGCAGCACATCTTTTTG
CCCTGGCGAGTCTAGCCGGATGGGCATCAAGGACAGAATCAGAATGGGCAGCAGCCAGCGGAGA
ACAGGCCCTTCTAAACAGCATCTGGCCCCTCCAACCATGCCTACAAGCCCTAGCTCTGAGCAAG
TGGGCGAAGCCACCTCTCCTACCAAGGTGCAGAAGTCCTGGTCCTTCAACGACCGGACCAGATT
CAGAGCCAGCCTGAGACTGAAGCCCAGAACCTCTGCCGAGGATGCCCCTTCTGAAGAGGTGGCC
GAAGAGAAGTCCTACCAGTGCGAGCTGACCGTGGACGACATCATGCCAGCCGTGAAAACCGTGA
TACGGTCTATCCGGATCCTGAAGTTCCTGGTGGCCAAGCGGAAGTTCAAAGAGACACTGCGGCC
CTACGACGTGAAGGACGTGATCGAGCAGTATTCTGCCGGCCACCTGGACATGCTGGGCAGAATC
AAGAGCCTGCAGACCAGAGTGGACCAGATCGTTGGAAGAGGCCCAGGCGACAGAAAGGCCAGAG
AGAAGGGCGATAAGGGCCCATCTGATGCCGAGGTTGTCGACGAGATATCAATGATGGGCAGAGT
GGTCAAGGTGGAAAAACAGGTGCAGAGCATCGAGCACAAGCTGGACCTGCTGCTGGGATTCTAC
AGCCGGTGTCTGAGAAGCGGCACATCTGCATCTCTGGGCGCTGTGCAGGTCCCACTGTTCGACC
CTGATATCACCAGCGACTATCACAGCCCCGTGGACCACGAGGACATCTCCGTTTCTGCTCAGAC
CCTGAGCATGAGCAGATCCGTGTCCACCAACATGGAC
Exemplary 3xFlag sequence
(SEQ ID NO: 145)
GACTACAAAGACCATGACGGTGATTATAAAGATCATGACATCGACTACAAGGATGACGATGACA
AG
Exemplary mScarlet sequence
(SEQ ID NO: 146)
ATGGTGAGCAAGGGCGAGGCAGTGATCAAGGAGTTCATGCGGTTCAAGGTGCACATGGAGGGCT
CCATGAACGGCCACGAGTTCGAGATCGAGGGCGAGGGCGAGGGCCGCCCCTACGAGGGCACCCA
GACCGCCAAGCTGAAGGTGACCAAGGGTGGCCCCCTGCCCTTCTCCTGGGACATCCTGTCCCCT
CAGTTCATGTACGGCTCCAGGGCCTTCATCAAGCACCCCGCCGACATCCCCGACTACTATAAGC
AGTCCTTCCCCGAGGGCTTCAAGTGGGAGCGCGTGATGAACTTCGAGGACGGCGGCGCCGTGAC
CGTGACCCAGGACACCTCCCTGGAGGACGGCACCCTGATCTACAAGGTGAAGCTCCGCGGCACC
AACTTCCCTCCTGACGGCCCCGTAATGCAGAAGAAGACAATGGGCTGGGAAGCGTCCACCGAGC
GGTTGTACCCCGAGGACGGCGTGCTGAAGGGCGACATTAAGATGGCCCTGCGCCTGAAGGACGG
CGGCCGCTACCTGGCGGACTTCAAGACCACCTACAAGGCCAAGAAGCCCGTGCAGATGCCCGGC
GCCTACAACGTCGACCGCAAGTTGGACATCACCTCCCACAACGAGGACTACACCGTGGTGGAAC
AGTACGAACGCTCCGAGGGCCGCCACTCCACCGGCGGCATGGACGAGCTGTACAAGTAA
Exemplary hKCNQ4 codon optimized sequence
(SEQ ID NO: 10)
ATGGCTGAAGCCCCTCCTAGAAGGCTTGGACTGGGACCTCCTCCTGGGGATGCTCCTAGAGCTG
AACTGGTGGCTCTGACAGCCGTGCAGTCTGAACAAGGCGAAGCTGGTGGCGGCGGATCTCCACG
TAGACTTGGACTGCTGGGAAGCCCTCTTCCTCCTGGTGCTCCACTTCCTGGACCTGGCAGTGGA
TCTGGATCTGCCTGTGGCCAGAGAAGCTCTGCCGCTCACAAGAGATACCGGCGGCTGCAGAACT
GGGTGTACAACGTGCTGGAAAGACCCAGAGGCTGGGCCTTCGTGTACCACGTGTTCATCTTTCT
GCTGGTGTTCAGCTGCCTGGTGCTGTCCGTGCTGAGCACCATCCAAGAACATCAAGAGCTGGCT
AACGAGTGCCTGTTAATACTGGAGTTTGTGATGATTGTGGTGTTCGGCCTCGAGTACATCGTCC
GCGTTTGGAGCGCCGGCTGCTGCTGCAGATATAGAGGTTGGCAAGGCAGATTCCGCTTCGCCAG
AAAGCCCTTCTGCGTGATCGACTTCATCGTGTTCGTGGCCAGCGTGGCCGTGATTGCTGCTGGC
ACACAGGGCAACATCTTCGCCACAAGCGCCCTGCGGAGCATGCGGTTTCTGCAGATCCTGAGAA
TGGTCCGAATGGACAGAAGAGGCGGCACCTGGAAGCTGCTGGGCTCTGTGGTGTACGCCCACAG
CAAAGAGCTGATCACCGCCTGGTACATCGGATTTCTGGTGCTGATCTTCGCCTCCTTCCTGGTG
TACCTGGCCGAGAAGGACGCCAACAGCGACTTTAGCAGCTACGCCGACTCTCTTTGGTGGGGCA
CCATCACACTGACCACCATCGGCTACGGCGACAAGACCCCTCACACATGGCTGGGAAGAGTGCT
GGCCGCTGGATTTGCTCTGCTGGGCATCAGCTTTTTCGCCCTGCCTGCCGGAATCCTCGGATCT
GGCTTTGCCCTGAAGGTGCAAGAGCAGCATAGACAAAAGCATTTTGAAAAGAGAAGAATGCCTG
CCGCCAACCTGATTCAGGCCGCTTGGAGACTGTACAGCACCGACATGAGCAGAGCCTACCTGAC
CGCCACGTGGTATTATTACGATTCGATCCTGCCTAGCTTCCGCGAACTGGCCCTGCTGTTTGAG
CATGTGCAGAGAGCCAGAAACGGCGGCCTCAGACCTCTGGAAGTTCGGAGAGCACCTGTGCCTG
ATGGCGCCCCTTCTAGATATCCTCCAGTGGCCACCTGTCACAGACCCGGCAGCACATCTTTTTG
CCCTGGCGAGTCTAGCCGGATGGGCATCAAGGACAGAATCAGAATGGGCAGCAGCCAGCGGAGA
ACAGGCCCTTCTAAACAGCATCTGGCCCCTCCAACCATGCCTACAAGCCCTAGCTCTGAGCAAG
TGGGCGAAGCCACCTCTCCTACCAAGGTGCAGAAGTCCTGGTCCTTCAACGACCGGACCAGATT
TAGAGCCAGCCTCCGGCTGAAGCCCAGAACCTCTGCCGAGGATGCCCCTTCTGAAGAGGTGGCC
GAAGAGAAGTCCTACCAGTGCGAGCTGACCGTGGACGACATCATGCCAGCCGTGAAAACCGTGA
TAAGGTCTATACGCATCCTGAAGTTCCTGGTGGCCAAGCGGAAGTTCAAAGAGACACTGCGGCC
CTACGACGTGAAGGACGTGATCGAGCAGTATTCTGCCGGCCACCTGGACATGCTGGGCAGAATC
AAGAGCCTGCAGACCAGAGTGGACCAGATCGTTGGAAGAGGCCCAGGCGACAGAAAGGCCAGAG
AGAAGGGCGATAAGGGCCCATCTGATGCCGAGGTTGTCGACGAGATATCAATGATGGGCAGAGT
CGTGAAAGTCGAAAAACAGGTGCAGAGCATCGAGCACAAGCTGGACCTGCTGCTGGGATTCTAC
AGCCGGTGTCTGAGAAGCGGCACATCTGCATCTCTGGGCGCTGTGCAGGTCCCACTGTTCGATC
CTGATATTACGAGCGATTATCACAGCCCCGTGGACCACGAGGACATCTCCGTTTCTGCTCAGAC
CCTGAGCATCAGCAGATCCGTGTCCACCAACATGGAC
Exemplary guide RNA hsammu386Fw sequence
(SEQ ID NO: 42)
AGGAGCACCAGGAACTTGCCA
Exemplary tracrRNA sequence
(SEQ ID NO: 147)
GTTTAAGTACTCTGTGCTGGAAACAGCACAGAATCTACTTAAACAAGGCAAAATGCCGTGTTTA
TCTCGTCAACTTGTTGGCGAGA
Exemplary U6shRNA-hKCNQ4-395 sequence
(SEQ ID NO: 148)
CTCCCCAGCTCCTGGGGGGAGCTGCTTGCCTCCCCCCGTTTTGAGAATCCTGATGGAGCggACT
GGTGAGGTAACAGGATCCGCCGTCCATCTTTATTCTCACTATGGGGCGAGGACTCAGCCGGCAC
CCTGTGCACAGCCAGCGAGGG
Exemplary Pol III transcriptional termination sequence
(SEQ ID NO: 149)
TTTTTT
Exemplary guide RNA U6-hsa408Rev sequence
(SEQ ID NO: 43)
AAGCCGAAAACCACGATCATC
Exemplary guide RNA U6-mmu408Rev sequence
(SEQ ID NO: 150)
AAGCCAAAGACCACAATCATC
Exemplary SV-40 Nuclear localization signal sequence
(SEQ ID NO: 151)
ATGGCCCCTAAGAAGAAGCGGAAAGTC
Exemplary catalytically inactive saCas9 coding sequence
(SEQ ID NO: 152)
AAGCGGAACTATATCCTGGGCCTCGCCATCGGCATCACCAGCGTTGGCTACGGCATCATCGACT
ACGAGACACGGGACGTGATCGATGCCGGCGTGCGGCTGTTTAAAGAGGCCAACGTCGAGAACAA
CGAGGGCCGCAGATCTAAGAGAGGCGCCAGACGGCTGAAGCGGAGAAGAAGGCACAGAATCCAG
AGAGTGAAGAAGCTGCTGTTCGACTACAACCTGCTGACCGACCACAGCGAGCTGAGCGGCATCA
ATCCTTACGAGGCCAGAGTGAAGGGCCTGAGCCAGAAGCTGAGCGAGGAAGAGTTTAGCGCCGC
TCTGCTGCACCTGGCCAAGAGAAGAGGCGTGCACAACGTGAACGAGGTGGAAGAGGACACCGGC
AACGAGCTGTCCACCAAAGAGCAGATCAGCCGGAACAGCAAGGCCCTGGAAGAGAAATACGTGG
CCGAACTGCAGCTGGAACGGCTGAAAAAGGATGGCGAAGTGCGGGGCAGCATCAATCGGTTCAA
GACCAGCGACTACGTGAAAGAAGCCAAACAGCTGCTGAAGGTGCAGAAGGCCTACCACCAGCTG
GACCAGAGCTTCATCGACACCTACATCGACCTGCTGGAAACCCGGCGGACCTACTATGAAGGAC
CTGGCGAGGGAAGCCCCTTCGGCTGGAAGGACATCAAAGAATGGTACGAGATGCTGATGGGCCA
CTGCACCTACTTTCCCGAGGAACTGCGGAGCGTGAAGTACGCCTACAACGCCGACCTGTACAAC
GCCCTGAACGACCTGAACAACCTCGTGATCACCCGGGACGAGAACGAGAAGCTGGAATATTACG
AGAAGTTCCAGATCATCGAGAACGTGTTCAAGCAGAAGAAGAAACCCACGCTGAAGCAGATCGC
CAAAGAGATCCTGGTCAACGAGGAAGATATCAAGGGCTACAGAGTGACCAGCACCGGCAAGCCC
GAGTTCACCAACCTGAAGGTGTACCACGACATCAAGGATATCACAGCCCGGAAAGAGATTATTG
AGAACGCCGAGCTGCTGGACCAAATCGCCAAGATCCTGACCATCTACCAGAGCAGCGAGGACAT
CCAAGAGGAACTGACCAATCTGAACTCCGAGCTGACCCAAGAAGAGATCGAGCAGATCTCTAAT
CTGAAGGGGTACACCGGCACACACAACCTGAGCCTGAAGGCCATCAACCTGATCCTGGACGAGC
TGTGGCACACCAACGACAACCAGATTGCCATCTTCAACAGGCTGAAGCTGGTGCCCAAGAAGGT
GGACCTGTCTCAGCAGAAAGAAATCCCCACCACACTGGTGGACGACTTCATTCTGAGCCCCGTG
GTCAAGAGGAGCTTCATCCAGAGCATCAAAGTGATCAACGCCATCATCAAGAAGTACGGGCTGC
CCAACGATATCATCATCGAGCTGGCCCGCGAGAAGAACTCCAAGGACGCCCAGAAAATGATCAA
CGAGATGCAGAAGAGGAACCGCCAGACCAACGAGCGGATCGAGGAAATCATCCGGACCACCGGC
AAAGAGAACGCCAAGTACCTGATCGAGAAGATCAAGCTGCACGACATGCAAGAGGGCAAGTGCC
TGTACAGCCTGGAAGCCATTCCTCTGGAAGATCTGCTGAACAATCCCTTCAACTATGAGGTGGA
CCACATCATCCCCAGAAGCGTGTCCTTCGACAACAGCTTCAACAACAAGGTGCTCGTGAAGCAA
GAGGAAGCCAGCAAGAAGGGCAACAGAACCCCATTCCAGTACCTGAGCAGCAGCGACAGCAAGA
TCAGCTACGAAACCTTCAAGAAGGACATCCTGAATCTGGCCAAAGGCAAGGGCAGAATCAGCAA
GACCAAGAAAGAATACCTGCTCGAGGAACGGGACATCAACAGATTCAGCGTGCAGAAAGACTTC
ATCAATCGGAACCTGGTGGACACCAGATACGCCACCAGAGGCCTGATGAATCTGCTGCGGAGCT
ACTTCAGAGTGAACAATCTGGACGTGAAAGTCAAGTCCATCAACGGCGGCTTCACCAGCTTTCT
GCGGCGGAAGTGGAAGTTCAAGAAAGAGCGGAACAAGGGCTATAAGCACCACGCCGAGGACGCC
CTGATCATTGCCAACGCCGATTTCATCTTCAAAGAGTGGAAGAAACTGGACAAGGCCAAAAAAG
TGATGGAAAACCAGATGTTCGAGGAAAAGCAGGCCGAGAGCATGCCCGAGATCGAAACCGAGCA
AGAGTACAAAGAGATTTTCATCACGCCCCACCAGATCAAGCACATTAAGGACTTCAAGGACTAG
AAGTACAGCCACCGCGTGGACAAGAAGCCCAATAGAGAGCTGATTAACGACACCCTGTACTCTA
CCCGGAAGGACGACAAGGGCAATACCCTGATCGTCAACAACCTGAACGGCCTGTACGACAAGGA
CAAGGACAAGCTCAAGAAGCTGATCAAGAAGAGCCCCGAGAAACTGCTGATGTACCACCACGAT
CCTCAGACCTACCAGAAACTGAAGCTCATCATGGAACAGTACGGCGACGAGAAGAATCCCCTGT
ACAAGTACTACGAGGAAACCGGGAACTACCTGACCAAGTACTCCAAAAAGGACAATGGGCCCGT
GATCAAGAAGATTAAGTATTACGGCAACAAGCTGAATGCCCACCTGGACATCACCGACGACTAC
CCCAACAGCAGAAACAAGGTGGTCAAGCTGTCCCTGAAGCCTTACAGATTCGACGTGTACCTGG
ACAACGGCGTGTAGAAGTTCGTGACCGTGAAGAACCTGGACGTCATCAAAAAAGAAAACTAGTA
CGAAGTCAACAGCAAGTGCTATGAGGAAGCCAAGAAACTCAAGAAAATCAGCAACCAGGCCGAG
TTTATCGCCTCCTTCTACAACAACGATCTGATCAAGATCAACGGGGAGCTGTATAGAGTGATCG
GCGTGAACAATGACCTGCTGAACCGGATCGAAGTGAACATGATCGACATCACCTACCGCGAGTA
CCTCGAGAACATGAACGATAAGCGGCCTCCACGGATCATTAAGACAATCGCCAGCAAGACGCAG
AGCATTAAGAAGTACAGCACAGACATCCTGGGCAACCTGTACGAAGTGAAGTCTAAGAAGCACC
CGCAGATTATCAAGAAAGGC
Exemplary SV-40 Nuclear localization sequence
(SEQ ID NO: 153)
CCCAAGAAAAAACGGAAGGTT
Exemplary catalytically active saCas9 coding sequence
(SEQ ID NO: 154)
AAGCGGAACTATATCCTGGGCCTCGACATCGGCATCACCAGCGTTGGCTACGGCATCATCGACT
ACGAGACACGGGACGTGATCGATGCCGGCGTGCGGCTGTTTAAAGAGGCCAACGTCGAGAACAA
CGAGGGCCGCAGATCTAAGAGAGGCGCCAGACGGCTGAAGCGGAGAAGAAGGCACAGAATCCAG
AGAGTGAAGAAGCTGCTGTTCGACTACAACCTGCTGACCGACCACAGCGAGCTGAGCGGCATCA
ATCCTTACGAGGCCAGAGTGAAGGGCCTGAGCCAGAAGCTGAGCGAGGAAGAGTTTAGCGCCGC
TCTGCTGCACCTGGCCAAGAGAAGAGGCGTGCACAACGTGAACGAGGTGGAAGAGGACACCGGC
AACGAGCTGTCCACCAAAGAGCAGATCAGCCGGAACAGCAAGGCCCTGGAAGAGAAATACGTGG
CCGAACTGCAGCTGGAACGGCTGAAAAAGGATGGCGAAGTGCGGGGCAGCATCAATCGGTTCAA
GACCAGCGACTACGTGAAAGAAGCCAAACAGCTGCTGAAGGTGCAGAAGGCCTACCACCAGCTG
GACCAGAGCTTCATCGACACCTACATCGACCTGCTGGAAACCCGGCGGACCTACTATGAAGGAC
CTGGCGAGGGAAGCCCCTTCGGCTGGAAGGACATCAAAGAATGGTACGAGATGCTGATGGGCCA
CTGCACCTACTTTCCCGAGGAACTGCGGAGCGTGAAGTACGCCTACAACGCCGACCTGTACAAC
GCCCTGAACGACCTGAACAACCTCGTGATCACCCGGGACGAGAACGAGAAGCTGGAATATTACG
AGAAGTTCCAGATCATCGAGAACGTGTTCAAGCAGAAGAAGAAACCCACGCTGAAGCAGATCGC
CAAAGAGATCCTGGTCAACGAGGAAGATATCAAGGGCTACAGAGTGACCAGCACCGGCAAGCCC
GAGTTCACCAACCTGAAGGTGTACCACGACATCAAGGATATCACAGCCCGGAAAGAGATTATTG
AGAACGCCGAGCTGCTGGACCAAATCGCCAAGATCCTGACCATCTACCAGAGCAGCGAGGACAT
CCAAGAGGAACTGACCAATCTGAACTCCGAGCTGACCCAAGAAGAGATCGAGCAGATCTCTAAT
CTGAAGGGGTACACCGGCACACACAACCTGAGCCTGAAGGCCATCAACCTGATCCTGGACGAGC
TGTGGCACACCAACGACAACCAGATTGCCATCTTCAACAGGCTGAAGCTGGTGCCCAAGAAGGT
GGACCTGTCTCAGCAGAAAGAAATCCCCACCACACTGGTGGACGACTTCATTCTGAGCCCCGTG
GTCAAGAGGAGCTTCATCCAGAGCATCAAAGTGATCAACGCCATCATCAAGAAGTACGGGCTGC
CCAACGATATCATCATCGAGCTGGCCCGCGAGAAGAACTCCAAGGACGCCCAGAAAATGATCAA
CGAGATGCAGAAGAGGAACCGCCAGACCAACGAGCGGATCGAGGAAATCATCCGGACCACCGGC
AAAGAGAACGCCAAGTACCTGATCGAGAAGATCAAGCTGCACGACATGCAAGAGGGCAAGTGCC
TGTACAGCCTGGAAGCCATTCCTCTGGAAGATCTGCTGAACAATCCCTTCAACTATGAGGTGGA
CCACATCATCCCCAGAAGCGTGTCCTTCGACAACAGCTTCAACAACAAGGTGCTCGTGAAGCAA
GAGGAAAACAGCAAGAAGGGCAACAGAACCCCATTCCAGTACCTGAGCAGCAGCGACAGCAAGA
TCAGCTACGAAACCTTCAAGAAGCACATCCTGAATCTGGCCAAAGGCAAGGGCAGAATCAGCAA
GACCAAGAAAGAATACCTGCTCGAGGAACGGGACATCAACAGATTCAGCGTGCAGAAAGACTTC
ATCAATCGGAACCTGGTGGACACCAGATACGCCACCAGAGGCCTGATGAATCTGCTGCGGAGCT
ACTTCAGAGTGAACAATCTGGACGTGAAAGTCAAGTCCATCAACGGCGGCTTCACCAGCTTTCT
GCGGCGGAAGTGGAAGTTCAAGAAAGAGCGGAACAAGGGCTATAAGCACCACGCCGAGGACGCC
CTGATCATTGCCAACGCCGATTTCATCTTCAAAGAGTGGAAGAAACTGGACAAGGCCAAAAAAG
TGATGGAAAACCAGATGTTCGAGGAAAAGCAGGCCGAGAGCATGCCCGAGATCGAAACCGAGCA
AGAGTACAAAGAGATTTTCATCACGCCCCACCAGATCAAGCACATTAAGGACTTCAAGGACTAG
AAGTACAGCCACCGCGTGGACAAGAAGCCCAATAGAGAGCTGATTAACGACACCCTGTACTCTA
CCCGGAAGGACGACAAGGGCAATACCCTGATCGTCAACAACCTGAACGGCCTGTACGACAAGGA
CAACGACAAGCTCAAGAAGCTGATCAACAAGAGCCCCGAGAAACTGCTGATGTACCACCACGAT
CCTCAGACCTACCAGAAACTGAAGCTCATCATGGAACAGTACGGCGACGAGAAGAATCCCCTGT
ACAAGTACTACGAGGAAACCGGGAACTACCTGACCAAGTACTCCAAAAAGGACAATGGGCCCGT
GATCAAGAAGATTAAGTATTACGGCAACAAGCTGAATGCCCACCTGGACATCACCGACGACTAC
CCCAACAGCAGAAACAAGGTGGTCAAGCTGTCCCTGAAGCCTTACAGATTCGACGTGTACCTGG
ACAACGGCGTGTAGAAGTTCGTGACCGTGAAGAACCTGGACGTCATCAAAAAAGAAAACTAGTA
CGAAGTCAACAGCAAGTGCTATGAGGAAGCCAAGAAACTCAAGAAAATCAGCAACCAGGCCGAG
TTTATCGCCTCCTTCTACAACAACGATCTGATCAAGATCAACGGGGAGCTGTATAGAGTGATCG
GCGTGAACAATGACCTGCTGAACCGGATCGAAGTGAACATGATCGACATCACCTACCGCGAGTA
CCTCGAGAACATGAACGATAAGCGGCCTCCACGGATCATTAAGACAATCGCCAGCAAGACGCAG
AGCATTAAGAAGTACAGCACAGACATCCTGGGCAACCTGTACGAAGTGAAGTCTAAGAAGCACC
CGCAGATTATCAAGAAAGGC
Exemplary guide RNA hsa233Fw sequence
(SEQ ID NO: 44)
GCGCTACCGCCGCCTGCAGAA
Exemplary guide RNA hsa447Fw sequence
(SEQ ID NO: 45)
TTTCGGCTTGGAGTACATCGT
Exemplary guide RNA hsa482Fw sequence
(SEQ ID NO: 46)
CCGGATGCTGCTGCCGCTACC
Exemplary guide RNA hsa4902Fw sequence
(SEQ ID NO: 47)
TGCTGCCGCTACCGAGGATGG

Exemplary Construct Cloning Sites

Exemplary Cloning site A sequence
(SEQ ID NO: 155)
GCGGCCGCACGCGT
Exemplary Cloning site C sequence
(SEQ ID NO: 156)
CTCCTGGGCAACGTGCTGGTTATTGTGACCGGTCGCTAGCCACC
Exemplary Cloning site D sequence
(SEQ ID NO: 157)
CCTGCAGGCGCCGGCG
Exemplary Cloning site E sequence
(SEQ ID NO: 158)
GAGCTCGCTGATCAGCCTCGA
Exemplary Cloning site F sequence
(SEQ ID NO: 159)
AAGCTTGAATTCAGCTGACGTGCCTCGGACCGCT
Exemplary Cloning site G sequence
(SEQ ID NO: 160)
GCGGCCGCACGCGTCTAGATCCCATATATGGAGTTCCG
Exemplary Cloning site H sequence
(SEQ ID NO: 161)
CAGATCGCCTGGAGACGCACCGGTGCCACC
Exemplary Cloning site I sequence
(SEQ ID NO: 162)
GGCTCCGGA
Exemplary Cloning site J sequence
(SEQ ID NO: 163)
GAGCTCGCTGATCAGCCTCGA
Exemplary Cloning site K sequence
(SEQ ID NO: 164)
TTAATT
Exemplary Cloning site L sequence
(SEQ ID NO: 165)
GGATCCCGGGCT
Exemplary Cloning site M sequence
(SEQ ID NO: 166)
TAAGAGCTCGCTGATCAGCCTCGA
Exemplary Cloning site N sequence
(SEQ ID NO: 167)
CTAAAGCTTGAATTCAAGCTTGAATTCAGCTGA
CGTGCCTCGGACCGCT
Exemplary Cloning site O sequence
(SEQ ID NO: 168)
GGCAGCGGC
Exemplary Cloning site P sequence
(SEQ ID NO: 169)
AGCGGCGCT
Exemplary Cloning site Q sequence
(SEQ ID NO: 170)
CTAAAGCTTGAATTCAGCTGACGTGCCTCGGACCGCT
Exemplary Cloning site R sequence
(SEQ ID NO: 171)
TAATAAGAGCTCGCTGATCAGCCTCGA

Exemplary miRNA Construct Sequences (Part 1)

Exemplary pITR.CAG.miR2-16_EGFP Construct sequence
(SEQ ID NO: 172)
CTGCGCGCTCGCTCGCTCACTGAGGCCGCCCGGGCGTCGGGCGACCTTTGGTCGCCCGGCCTCA
GTGAGCGAGCGAGCGCGCAGAGAGGGAGTGGCCAACTCCATCACTAGGGGTTCCTGCGGCCGCA
CGCGTGACATTGATTATTGACTAGTTATTAATAGTAATCAATTACGGGGTCATTAGTTCATAGC
CCATATATGGAGTTCCGCGTTACATAACTTACGGTAAATGGCCCGCCTGGCTGACCGCCCAACG
ACCCCCGCCCATTGACGTCAATAATGACGTATGTTCCCATAGTAACGCCAATAGGGACTTTCCA
TTGACGTCAATGGGTGGACTATTTACGGTAAACTGCCCACTTGGCAGTACATCAAGTGTATCAT
ATGCCAAGTACGCCCCCTATTGACGTCAATGACGGTAAATGGCCCGCCTGGCATTATGCCCAGT
ACATGACCTTATGGGACTTTCCTACTTGGCAGTACATCTACGTATTAGTCATCGCTATTACCAT
GGGTCGAGGTGAGCCCCACGTTCTGCTTCACTCTCCCCATCTCCCCCCCCTCCCCACCCCCAAT
TTTGTATTTATTTATTTTTTAATTATTTTGTGCAGCGATGGGGGCGGGGGGGGGGGGGGCGCGC
GCCAGGCGGGGCGGGGCGGGGCGAGGGGCGGGGCGGGGCGAGGCGGAGAGGTGCGGCGGCAGCC
AATCAGAGCGGCGCGCTCCGAAAGTTTCCTTTTATGGCGAGGCGGCGGCGGCGGCGGCCCTATA
AAAAGCGAAGCGCGCGGCGGGCGGGAGTCGCTGCGTTGCCTTCGCCCCGTGCCCCGCTCCGCGC
CGCCTCGCGCCGCCCGCCCCGGCTCTGACTGACCGCGTTACTCCCACAGGTGAGCGGGCGGGAC
GGCCCTTCTCCTCCGGGCTGTAATTAGCGCTTGGTTTAATGACGGCTCGTTTCTTTTCTGTGGC
TGCGTGAAAGCCTTAAAGGGCTCCGGGAGGGCCCTTTGTGCGGGGGGGAGCGGCTCGGGGGGTG
CGTGCGTGTGTGTGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGCGGCCGGGGGCGG
TGCCCCGCGGTGCGGGGGGGCTGCGAGGGGAACAAAGGCTGCGTGCGGGGTGTGTGCGTGGGGG
GGTGAGCAGGGGGTGTGGGCGCGGCGGTCGGGCTGTAACCCCCCCCTGCACCCCCCTCCCCGAG
TTGCTGAGCACGGCCCGGCTTCGGGTGCGGGGCTCCGTGCGGGGCGTGGCGCGGGGCTCGCCGT
GCCGGGCGGGGGGTGGCGGCAGGTGGGGGTGCCGGGCGGGGCGGGGCCGCCTCGGGCCGGGGAG
GGCTCGGGGGAGGGGCGCGGCGGCCCCCGGAGCGCCGGCGCATAGCTCTTATGATAGCAATGTC
AGCAGTGCCTTTGAGTCTCAGAGATGCCCGGGTTAAGATTCTAAAATTATCACGGGTATTACTT
GAGGCTCAGAAGTAAGGTTGACCATACTCTACAGTTGTGTCTAGAGCCTCTGCTAACCATGTTC
ATGCCTTCTTCTTTTTCCTACAGCTCCTGGGCAACGTGCTGGTTATTGTGACCGGTCGCTAGCC
ACCATGGTGAGCAAGGGCGAGGAGCTGTTCACCGGGGTGGTGCCCATCCTGGTCGAGCTGGACG
GCGACGTAAACGGCCACAAGTTCAGCGTGTCCGGCGAGGGCGAGGGCGATGCCACCTACGGCAA
GCTGACCCTGAAGTTCATCTGCACCACCGGCAAGCTGCCCGTGCCCTGGCCCACCCTCGTGACC
ACCCTGACCTACGGCGTGCAGTGCTTCAGCCGCTACCCCGACCACATGAAGCAGCACGACTTCT
TCAAGTCCGCCATGCCCGAAGGCTACGTCCAGGAGCGCACCATCTTCTTCAAGGACGACGGCAA
CTACAAGACCCGCGCCGAGGTGAAGTTCGAGGGCGACACCCTGGTGAACCGCATCGAGCTGAAG
GGCATCGACTTCAAGGAGGACGGCAACATCCTGGGGCACAAGCTGGAGTACAACTACAACAGCC
ACAACGTCTATATCATGGCCGACAAGCAGAAGAACGGCATCAAGGTGAACTTCAAGATCCGCCA
CAACATCGAGGACGGCAGCGTGCAGCTCGCCGACCACTACCAGCAGAACACCCCCATCGGCGAC
GGCCCCGTGCTGCTGCCCGACAACCACTACCTGAGCACCCAGTCCGCCCTGAGCAAAGACCCCA
ACGAGAAGCGCGATCACATGGTCCTGCTGGAGTTCGTGACCGCCGCCGGGATCACTCTCGGCAT
GGACGAGCTGTACAAGTAAcctgcaggcgccggcgCATAGCTCTTATGATAGCAATGTCAGCAG
TGCCTTTGAGTCTCAGAGATGCCCGGGTTAAGATTCTAAAATTATCACGGGTATTACTTGAGGC
TCAGAAGTAAGGTTGACCATACTCTACAGTTGTGGAGCTCGCTGATCAGCCTCGACTGTGCCTT
CTAGTTGCCAGCCATCTGTTGTTTGCCCCTCCCCCGTGCCTTCCTTGACCCTGGAAGGTGCCAC
TCCCACTGTCCTTTCCTAATAAAATGAGGAAATTGCATCGCATTGTCTGAGTAGGTGTCATTCT
ATTCTGGGGGGTGGGGTGGGGCAGGACAGCAAGGGGGAGGATTGGGAAGACAATAGCAGGCATG
CTGGGGATGCGGTGGGCTCTATGGAAGCTTGAATTCAGCTGACGTGCCTCGGACCGCTAGGAAC
CCCTAGTGATGGAGTTGGCCACTCCCTCTCTGCGCGCTCGCTCGCTCACTGAGGCCGGGCGACC
AAAGGTCGCCCGACGCCCGGGCTTTGCCCGGGCGGCCTCAGTGAGCGAGCGAGCGCGCAG
Exemplary pITR.CAG.miR2-26_EGFP Construct sequence
(SEQ ID NO: 173)
CTGCGCGCTCGCTCGCTCACTGAGGCCGCCCGGGCGTCGGGCGACCTTTGGTCGCCCGGCCTCA
GTGAGCGAGCGAGCGCGCAGAGAGGGAGTGGCCAACTCCATCACTAGGGGTTCCTGCGGCCGCA
CGCGTGACATTGATTATTGACTAGTTATTAATAGTAATCAATTACGGGGTCATTAGTTCATAGC
CCATATATGGAGTTCCGCGTTACATAACTTACGGTAAATGGCCCGCCTGGCTGACCGCCCAACG
ACCCCCGCCCATTGACGTCAATAATGACGTATGTTCCCATAGTAACGCCAATAGGGACTTTCCA
TTGACGTCAATGGGTGGACTATTTACGGTAAACTGCCCACTTGGCAGTACATCAAGTGTATCAT
ATGCCAAGTACGCCCCCTATTGACGTCAATGACGGTAAATGGCCCGCCTGGCATTATGCCCAGT
ACATGACCTTATGGGACTTTCCTACTTGGCAGTACATCTACGTATTAGTCATCGCTATTACCAT
GGGTCGAGGTGAGCCCCACGTTCTGCTTCACTCTCCCCATCTCCCCCCCCTCCCCACCCCCAAT
TTTGTATTTATTTATTTTTTAATTATTTTGTGCAGCGATGGGGGCGGGGGGGGGGGGGGCGCGC
GCCAGGCGGGGCGGGGCGGGGCGAGGGGCGGGGCGGGGCGAGGCGGAGAGGTGCGGCGGCAGCC
AATCAGAGCGGCGCGCTCCGAAAGTTTCCTTTTATGGCGAGGCGGCGGCGGCGGCGGCCCTATA
AAAAGCGAAGCGCGCGGCGGGCGGGAGTCGCTGCGTTGCCTTCGCCCCGTGCCCCGCTCCGCGC
CGCCTCGCGCCGCCCGCCCCGGCTCTGACTGACCGCGTTACTCCCACAGGTGAGCGGGCGGGAC
GGCCCTTCTCCTCCGGGCTGTAATTAGCGCTTGGTTTAATGACGGCTCGTTTCTTTTCTGTGGC
TGCGTGAAAGCCTTAAAGGGCTCCGGGAGGGCCCTTTGTGCGGGGGGGAGCGGCTCGGGGGGTG
CGTGCGTGTGTGTGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGCGGCCGGGGGCGG
TGCCCCGCGGTGCGGGGGGGCTGCGAGGGGAACAAAGGCTGCGTGCGGGGTGTGTGCGTGGGGG
GGTGAGCAGGGGGTGTGGGCGCGGCGGTCGGGCTGTAACCCCCCCCTGCACCCCCCTCCCCGAG
TTGCTGAGCACGGCCCGGCTTCGGGTGCGGGGCTCCGTGCGGGGCGTGGCGCGGGGCTCGCCGT
GCCGGGCGGGGGGTGGCGGCAGGTGGGGGTGCCGGGCGGGGCGGGGCCGCCTCGGGCCGGGGAG
GGCTCGGGGGAGGGGCGCGGCGGCCCCCGGAGCGCCGGCGGAGCAGGAGGACCAAGGCCCTGGC
GAAGGCCGTGGCCTCGTTGAGTCTCAGAGATGCCCGGTGTGCAGGTCCCAATGGCCGGGTATTA
CTGAGATTCCACGGGGACGCGGGCCTGGACGCCGGCATCCGGGCTCAGGACCCCCCTCTCTAGA
GCCTCTGCTAACCATGTTCATGCCTTCTTCTTTTTCCTACAGCTCCTGGGCAACGTGCTGGTTA
TTGTGACCGGTCGCTAGCCACCATGGTGAGCAAGGGCGAGGAGCTGTTCACCGGGGTGGTGCCC
ATCCTGGTCGAGCTGGACGGCGACGTAAACGGCCACAAGTTCAGCGTGTCCGGCGAGGGCGAGG
GCGATGCCACCTACGGCAAGCTGACCCTGAAGTTCATCTGCACCACCGGCAAGCTGCCCGTGCC
CTGGCCCACCCTCGTGACCACCCTGACCTACGGCGTGCAGTGCTTCAGCCGCTACCCCGACCAC
ATGAAGCAGCACGACTTCTTCAAGTCCGCCATGCCCGAAGGCTACGTCCAGGAGCGCACCATCT
TCTTCAAGGACGACGGCAACTACAAGACCCGCGCCGAGGTGAAGTTCGAGGGCGACACCCTGGT
GAACCGCATCGAGCTGAAGGGCATCGACTTCAAGGAGGACGGCAACATCCTGGGGCACAAGCTG
GAGTACAACTACAACAGCCACAACGTCTATATCATGGCCGACAAGCAGAAGAACGGCATCAAGG
TGAACTTCAAGATCCGCCACAACATCGAGGACGGCAGCGTGCAGCTCGCCGACCACTACCAGCA
GAACACCCCCATCGGCGACGGCCCCGTGCTGCTGCCCGACAACCACTACCTGAGCACCCAGTCC
GCCCTGAGCAAAGACCCCAACGAGAAGCGCGATCACATGGTCCTGCTGGAGTTCGTGACCGCCG
CCGGGATCACTCTCGGCATGGACGAGCTGTACAAGTAAcctgcaggcgccggcgGAGCAGGAGG
ACCAAGGCCCTGGCGAAGGCCGTGGCCTCGTTGAGTCTCAGAGATGCCCGGTGTGCAGGTCCCA
ATGGCCGGGTATTACTGAGATTCCACGGGGACGCGGGCCTGGACGCCGGCATCCGGGCTCAGGA
CCCCCCTCGAGCTCGCTGATCAGCCTCGACTGTGCCTTCTAGTTGCCAGCCATCTGTTGTTTGC
CCCTCCCCCGTGCCTTCCTTGACCCTGGAAGGTGCCACTCCCACTGTCCTTTCCTAATAAAATG
AGGAAATTGCATCGCATTGTCTGAGTAGGTGTCATTCTATTCTGGGGGGTGGGGTGGGGCAGGA
CAGCAAGGGGGAGGATTGGGAAGACAATAGCAGGCATGCTGGGGATGCGGTGGGCTCTATGGAA
GCTTGAATTCAGCTGACGTGCCTCGGACCGCTAGGAACCCCTAGTGATGGAGTTGGCCACTCCC
TCTCTGCGCGCTCGCTCGCTCACTGAGGCCGGGCGACCAAAGGTCGCCCGACGCCCGGGCTTTG
CCCGGGCGGCCTCAGTGAGCGAGCGAGCGCGCAG
Exemplary pITR.CAG.miR2-96_EGFP Construct sequence
(SEQ ID NO: 174)
CTGCGCGCTCGCTCGCTCACTGAGGCCGCCCGGGCGTCGGGCGACCTTTGGTCGCCCGGCCTCA
GTGAGCGAGCGAGCGCGCAGAGAGGGAGTGGCCAACTCCATCACTAGGGGTTCCTGCGGCCGCA
CGCGTGACATTGATTATTGACTAGTTATTAATAGTAATCAATTACGGGGTCATTAGTTCATAGC
CCATATATGGAGTTCCGCGTTACATAACTTACGGTAAATGGCCCGCCTGGCTGACCGCCCAACG
ACCCCCGCCCATTGACGTCAATAATGACGTATGTTCCCATAGTAACGCCAATAGGGACTTTCCA
TTGACGTCAATGGGTGGACTATTTACGGTAAACTGCCCACTTGGCAGTACATCAAGTGTATCAT
ATGCCAAGTACGCCCCCTATTGACGTCAATGACGGTAAATGGCCCGCCTGGCATTATGCCCAGT
ACATGACCTTATGGGACTTTCCTACTTGGCAGTACATCTACGTATTAGTCATCGCTATTACCAT
GGGTCGAGGTGAGCCCCACGTTCTGCTTCACTCTCCCCATCTCCCCCCCCTCCCCACCCCCAAT
TTTGTATTTATTTATTTTTTAATTATTTTGTGCAGCGATGGGGGCGGGGGGGGGGGGGGCGCGC
GCCAGGCGGGGCGGGGCGGGGCGAGGGGCGGGGCGGGGCGAGGCGGAGAGGTGCGGCGGCAGCC
AATCAGAGCGGCGCGCTCCGAAAGTTTCCTTTTATGGCGAGGCGGCGGCGGCGGCGGCCCTATA
AAAAGCGAAGCGCGCGGCGGGCGGGAGTCGCTGCGTTGCCTTCGCCCCGTGCCCCGCTCCGCGC
CGCCTCGCGCCGCCCGCCCCGGCTCTGACTGACCGCGTTACTCCCACAGGTGAGCGGGCGGGAC
GGCCCTTCTCCTCCGGGCTGTAATTAGCGCTTGGTTTAATGACGGCTCGTTTCTTTTCTGTGGC
TGCGTGAAAGCCTTAAAGGGCTCCGGGAGGGCCCTTTGTGCGGGGGGGAGCGGCTCGGGGGGTG
CGTGCGTGTGTGTGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGCGGCCGGGGGCGG
TGCCCCGCGGTGCGGGGGGGCTGCGAGGGGAACAAAGGCTGCGTGCGGGGTGTGTGCGTGGGGG
GGTGAGCAGGGGGTGTGGGCGCGGCGGTCGGGCTGTAACCCCCCCCTGCACCCCCCTCCCCGAG
TTGCTGAGCACGGCCCGGCTTCGGGTGCGGGGCTCCGTGCGGGGCGTGGCGCGGGGCTCGCCGT
GCCGGGCGGGGGGTGGCGGCAGGTGGGGGTGCCGGGCGGGGCGGGGCCGCCTCGGGCCGGGGAG
GGCTCGGGGGAGGGGCGCGGCGGCCCCCGGAGCGCCGGCGGTGTCCCCAGAGAGCCCGCACCAG
TGCCATCTGCTTGGCCGATTTGAGTCTCAGAGATGCCCggCTTGTGTCTCTCCGCTCTGAGCTG
TCTGTCTCGAGGCTCATATGGGAAAAGCAGGACCCGCAGCTGCGTCCGCCTCCCCTGCATTCTA
GAGCCTCTGCTAACCATGTTCATGCCTTCTTCTTTTTCCTACAGCTCCTGGGCAACGTGCTGGT
TATTGTGACCGGTCGCTAGCCACCATGGTGAGCAAGGGCGAGGAGCTGTTCACCGGGGTGGTGC
CCATCCTGGTCGAGCTGGACGGCGACGTAAACGGCCACAAGTTCAGCGTGTCCGGCGAGGGCGA
GGGCGATGCCACCTACGGCAAGCTGACCCTGAAGTTCATCTGCACCACCGGCAAGCTGCCCGTG
CCCTGGCCCACCCTCGTGACCACCCTGACCTACGGCGTGCAGTGCTTCAGCCGCTACCCCGACC
ACATGAAGCAGCACGACTTCTTCAAGTCCGCCATGCCCGAAGGCTACGTCCAGGAGCGCACCAT
CTTCTTCAAGGACGACGGCAACTACAAGACCCGCGCCGAGGTGAAGTTCGAGGGCGACACCCTG
GTGAACCGCATCGAGCTGAAGGGCATCGACTTCAAGGAGGACGGCAACATCCTGGGGCACAAGC
TGGAGTACAACTACAACAGCCACAACGTCTATATCATGGCCGACAAGCAGAAGAACGGCATCAA
GGTGAACTTCAAGATCCGCCACAACATCGAGGACGGCAGCGTGCAGCTCGCCGACCACTACCAG
CAGAACACCCCCATCGGCGACGGCCCCGTGCTGCTGCCCGACAACCACTACCTGAGCACCCAGT
CCGCCCTGAGCAAAGACCCCAACGAGAAGCGCGATCACATGGTCCTGCTGGAGTTCGTGACCGC
CGCCGGGATCACTCTCGGCATGGACGAGCTGTACAAGTAAcctgcaggcgccggcgGTGTCCCC
AGAGAGCCCGCACCAGTGCCATCTGCTTGGCCGATTTGAGTCTCAGAGATGCCCggCTTGTGTC
TCTCCGCTCTGAGCTGTCTGTCTCGAGGCTCATATGGGAAAAGCAGGACCCGCAGCTGCGTCCG
CCTCCCCTGCATGAGCTCGCTGATCAGCCTCGACTGTGCCTTCTAGTTGCCAGCCATCTGTTGT
TTGCCCCTCCCCCGTGCCTTCCTTGACCCTGGAAGGTGCCACTCCCACTGTCCTTTCCTAATAA
AATGAGGAAATTGCATCGCATTGTCTGAGTAGGTGTCATTCTATTCTGGGGGGTGGGGTGGGGC
AGGACAGCAAGGGGGAGGATTGGGAAGACAATAGCAGGCATGCTGGGGATGCGGTGGGCTCTAT
GGAAGCTTGAATTCAGCTGACGTGCCTCGGACCGCTAGGAACCCCTAGTGATGGAGTTGGCCAC
TCCCTCTCTGCGCGCTCGCTCGCTCACTGAGGCCGGGCGACCAAAGGTCGCCCGACGCCCGGGC
TTTGCCCGGGCGGCCTCAGTGAGCGAGCGAGCGCGCAG
Exemplary pITR.CAG.miR2i_miR4u-16_EGFP Construct sequence
(SEQ ID NO: 175)
CTGCGCGCTCGCTCGCTCACTGAGGCCGCCCGGGCGTCGGGCGACCTTTGGTCGCCCGGCCTCA
GTGAGCGAGCGAGCGCGCAGAGAGGGAGTGGCCAACTCCATCACTAGGGGTTCCTGCGGCCGCA
CGCGTGACATTGATTATTGACTAGTTATTAATAGTAATCAATTACGGGGTCATTAGTTCATAGC
CCATATATGGAGTTCCGCGTTACATAACTTACGGTAAATGGCCCGCCTGGCTGACCGCCCAACG
ACCCCCGCCCATTGACGTCAATAATGACGTATGTTCCCATAGTAACGCCAATAGGGACTTTCCA
TTGACGTCAATGGGTGGACTATTTACGGTAAACTGCCCACTTGGCAGTACATCAAGTGTATCAT
ATGCCAAGTACGCCCCCTATTGACGTCAATGACGGTAAATGGCCCGCCTGGCATTATGCCCAGT
ACATGACCTTATGGGACTTTCCTACTTGGCAGTACATCTACGTATTAGTCATCGCTATTACCAT
GGGTCGAGGTGAGCCCCACGTTCTGCTTCACTCTCCCCATCTCCCCCCCCTCCCCACCCCCAAT
TTTGTATTTATTTATTTTTTAATTATTTTGTGCAGCGATGGGGGCGGGGGGGGGGGGGGCGCGC
GCCAGGCGGGGCGGGGCGGGGCGAGGGGCGGGGCGGGGCGAGGCGGAGAGGTGCGGCGGCAGCC
AATCAGAGCGGCGCGCTCCGAAAGTTTCCTTTTATGGCGAGGCGGCGGCGGCGGCGGCCCTATA
AAAAGCGAAGCGCGCGGCGGGCGGGAGTCGCTGCGTTGCCTTCGCCCCGTGCCCCGCTCCGCGC
CGCCTCGCGCCGCCCGCCCCGGCTCTGACTGACCGCGTTACTCCCACAGGTGAGCGGGCGGGAC
GGCCCTTCTCCTCCGGGCTGTAATTAGCGCTTGGTTTAATGACGGCTCGTTTCTTTTCTGTGGC
TGCGTGAAAGCCTTAAAGGGCTCCGGGAGGGCCCTTTGTGCGGGGGGGAGCGGCTCGGGGGGTG
CGTGCGTGTGTGTGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGCGGCCGGGGGCGG
TGCCCCGCGGTGCGGGGGGGCTGCGAGGGGAACAAAGGCTGCGTGCGGGGTGTGTGCGTGGGGG
GGTGAGCAGGGGGTGTGGGCGCGGCGGTCGGGCTGTAACCCCCCCCTGCACCCCCCTCCCCGAG
TTGCTGAGCACGGCCCGGCTTCGGGTGCGGGGCTCCGTGCGGGGCGTGGCGCGGGGCTCGCCGT
GCCGGGCGGGGGGTGGCGGCAGGTGGGGGTGCCGGGCGGGGCGGGGCCGCCTCGGGCCGGGGAG
GGCTCGGGGGAGGGGCGCGGCGGCCCCCGGAGCGCCGGCGCATAGCTCTTATGATAGCAATGTC
AGCAGTGCCTTTGAGTCTCAGAGATGCCCGGGTTAAGATTCTAAAATTATCACGGGTATTACTT
GAGGCTCAGAAGTAAGGTTGACCATACTCTACAGTTGTGTCTAGAGCCTCTGCTAACCATGTTC
ATGCCTTCTTCTTTTTCCTACAGCTCCTGGGCAACGTGCTGGTTATTGTGACCGGTCGCTAGCC
ACCATGGTGAGCAAGGGCGAGGAGCTGTTCACCGGGGTGGTGCCCATCCTGGTCGAGCTGGACG
GCGACGTAAACGGCCACAAGTTCAGCGTGTCCGGCGAGGGCGAGGGCGATGCCACCTACGGCAA
GCTGACCCTGAAGTTCATCTGCACCACCGGCAAGCTGCCCGTGCCCTGGCCCACCCTCGTGACC
ACCCTGACCTACGGCGTGCAGTGCTTCAGCCGCTACCCCGACCACATGAAGCAGCACGACTTCT
TCAAGTCCGCCATGCCCGAAGGCTACGTCCAGGAGCGCACCATCTTCTTCAAGGACGACGGCAA
CTACAAGACCCGCGCCGAGGTGAAGTTCGAGGGCGACACCCTGGTGAACCGCATCGAGCTGAAG
GGCATCGACTTCAAGGAGGACGGCAACATCCTGGGGCACAAGCTGGAGTACAACTACAACAGCC
ACAACGTCTATATCATGGCCGACAAGCAGAAGAACGGCATCAAGGTGAACTTCAAGATCCGCCA
CAACATCGAGGACGGCAGCGTGCAGCTCGCCGACCACTACCAGCAGAACACCCCCATCGGCGAC
GGCCCCGTGCTGCTGCCCGACAACCACTACCTGAGCACCCAGTCCGCCCTGAGCAAAGACCCCA
ACGAGAAGCGCGATCACATGGTCCTGCTGGAGTTCGTGACCGCCGCCGGGATCACTCTCGGCAT
GGACGAGCTGTACAAGTAAcctgcaggcgccggcgCATAGCTCTTATGATAGCAATGTCAGCAG
TGCCTTTCTCGAAGTGCTTCTGCCGGGTTAAGATTCTAAAATTATCACGGTAGAAACATCTTCG
GGAGAAGTAAGGTTGACCATACTCTACAGTTGTGGAGCTCGCTGATCAGCCTCGACTGTGCCTT
CTAGTTGCCAGCCATCTGTTGTTTGCCCCTCCCCCGTGCCTTCCTTGACCCTGGAAGGTGCCAC
TCCCACTGTCCTTTCCTAATAAAATGAGGAAATTGCATCGCATTGTCTGAGTAGGTGTCATTCT
ATTCTGGGGGGTGGGGTGGGGCAGGACAGCAAGGGGGAGGATTGGGAAGACAATAGCAGGCATG
CTGGGGATGCGGTGGGCTCTATGGAAGCTTGAATTCAGCTGACGTGCCTCGGACCGCTAGGAAC
CCCTAGTGATGGAGTTGGCCACTCCCTCTCTGCGCGCTCGCTCGCTCACTGAGGCCGGGCGACC
AAAGGTCGCCCGACGCCCGGGCTTTGCCCGGGCGGCCTCAGTGAGCGAGCGAGCGCGCAG
Exemplary pITR.CAG.miR2i_miR4u-26_EGFP Construct sequence
(SEQ ID NO: 176)
CTGCGCGCTCGCTCGCTCACTGAGGCCGCCCGGGCGTCGGGCGACCTTTGGTCGCCCGGCCTCA
GTGAGCGAGCGAGCGCGCAGAGAGGGAGTGGCCAACTCCATCACTAGGGGTTCCTGCGGCCGCA
CGCGTGACATTGATTATTGACTAGTTATTAATAGTAATCAATTACGGGGTCATTAGTTCATAGC
CCATATATGGAGTTCCGCGTTACATAACTTACGGTAAATGGCCCGCCTGGCTGACCGCCCAACG
ACCCCCGCCCATTGACGTCAATAATGACGTATGTTCCCATAGTAACGCCAATAGGGACTTTCCA
TTGACGTCAATGGGTGGACTATTTACGGTAAACTGCCCACTTGGCAGTACATCAAGTGTATCAT
ATGCCAAGTACGCCCCCTATTGACGTCAATGACGGTAAATGGCCCGCCTGGCATTATGCCCAGT
ACATGACCTTATGGGACTTTCCTACTTGGCAGTACATCTACGTATTAGTCATCGCTATTACCAT
GGGTCGAGGTGAGCCCCACGTTCTGCTTCACTCTCCCCATCTCCCCCCCCTCCCCACCCCCAAT
TTTGTATTTATTTATTTTTTAATTATTTTGTGCAGCGATGGGGGCGGGGGGGGGGGGGGCGCGC
GCCAGGCGGGGCGGGGCGGGGCGAGGGGCGGGGCGGGGCGAGGCGGAGAGGTGCGGCGGCAGCC
AATCAGAGCGGCGCGCTCCGAAAGTTTCCTTTTATGGCGAGGCGGCGGCGGCGGCGGCCCTATA
AAAAGCGAAGCGCGCGGCGGGCGGGAGTCGCTGCGTTGCCTTCGCCCCGTGCCCCGCTCCGCGC
CGCCTCGCGCCGCCCGCCCCGGCTCTGACTGACCGCGTTACTCCCACAGGTGAGCGGGCGGGAC
GGCCCTTCTCCTCCGGGCTGTAATTAGCGCTTGGTTTAATGACGGCTCGTTTCTTTTCTGTGGC
TGCGTGAAAGCCTTAAAGGGCTCCGGGAGGGCCCTTTGTGCGGGGGGGAGCGGCTCGGGGGGTG
CGTGCGTGTGTGTGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGCGGCCGGGGGCGG
TGCCCCGCGGTGCGGGGGGGCTGCGAGGGGAACAAAGGCTGCGTGCGGGGTGTGTGCGTGGGGG
GGTGAGCAGGGGGTGTGGGCGCGGCGGTCGGGCTGTAACCCCCCCCTGCACCCCCCTCCCCGAG
TTGCTGAGCACGGCCCGGCTTCGGGTGCGGGGCTCCGTGCGGGGCGTGGCGCGGGGCTCGCCGT
GCCGGGCGGGGGGTGGCGGCAGGTGGGGGTGCCGGGCGGGGCGGGGCCGCCTCGGGCCGGGGAG
GGCTCGGGGGAGGGGCGCGGCGGCCCCCGGAGCGCCGGCGGAGCAGGAGGACCAAGGCCCTGGC
GAAGGCCGTGGCCTCGTTGAGTCTCAGAGATGCCCGGTGTGCAGGTCCCAATGGCCGGGTATTA
CTGAGATTCCACGGGGACGCGGGCCTGGACGCCGGCATCCGGGCTCAGGACCCCCCTCTCTAGA
GCCTCTGCTAACCATGTTCATGCCTTCTTCTTTTTCCTACAGCTCCTGGGCAACGTGCTGGTTA
TTGTGACCGGTCGCTAGCCACCATGGTGAGCAAGGGCGAGGAGCTGTTCACCGGGGTGGTGCCC
ATCCTGGTCGAGCTGGACGGCGACGTAAACGGCCACAAGTTCAGCGTGTCCGGCGAGGGCGAGG
GCGATGCCACCTACGGCAAGCTGACCCTGAAGTTCATCTGCACCACCGGCAAGCTGCCCGTGCC
CTGGCCCACCCTCGTGACCACCCTGACCTACGGCGTGCAGTGCTTCAGCCGCTACCCCGACCAC
ATGAAGCAGCACGACTTCTTCAAGTCCGCCATGCCCGAAGGCTACGTCCAGGAGCGCACCATCT
TCTTCAAGGACGACGGCAACTACAAGACCCGCGCCGAGGTGAAGTTCGAGGGCGACACCCTGGT
GAACCGCATCGAGCTGAAGGGCATCGACTTCAAGGAGGACGGCAACATCCTGGGGCACAAGCTG
GAGTACAACTACAACAGCCACAACGTCTATATCATGGCCGACAAGCAGAAGAACGGCATCAAGG
TGAACTTCAAGATCCGCCACAACATCGAGGACGGCAGCGTGCAGCTCGCCGACCACTACCAGCA
GAACACCCCCATCGGCGACGGCCCCGTGCTGCTGCCCGACAACCACTACCTGAGCACCCAGTCC
GCCCTGAGCAAAGACCCCAACGAGAAGCGCGATCACATGGTCCTGCTGGAGTTCGTGACCGCCG
CCGGGATCACTCTCGGCATGGACGAGCTGTACAAGTAAcctgcaggcgccggcgGAGCAGGAGG
ACCAAGGCCCTGGCGAAGGCCGTGGCCTCGTTCTCGAAGTGCTTCTGCCGGTGTGCAGGTCCCA
ATGGCCGGTAGAATCGTTTCGAGCACGGGGACGCGGGCCTGGACGCCGGCATCCGGGCTCAGGA
CCCCCCTCGAGCTCGCTGATCAGCCTCGACTGTGCCTTCTAGTTGCCAGCCATCTGTTGTTTGC
CCCTCCCCCGTGCCTTCCTTGACCCTGGAAGGTGCCACTCCCACTGTCCTTTCCTAATAAAATG
AGGAAATTGCATCGCATTGTCTGAGTAGGTGTCATTCTATTCTGGGGGGTGGGGTGGGGCAGGA
CAGCAAGGGGGAGGATTGGGAAGACAATAGCAGGCATGCTGGGGATGCGGTGGGCTCTATGGAA
GCTTGAATTCAGCTGACGTGCCTCGGACCGCTAGGAACCCCTAGTGATGGAGTTGGCCACTCCC
TCTCTGCGCGCTCGCTCGCTCACTGAGGCCGGGCGACCAAAGGTCGCCCGACGCCCGGGCTTTG
CCCGGGCGGCCTCAGTGAGCGAGCGAGCGCGCAG
Exemplary pITR.CAG.miR2i_miR4u-96_EGFP Construct sequence
(SEQ ID NO: 177)
CTGCGCGCTCGCTCGCTCACTGAGGCCGCCCGGGCGTCGGGCGACCTTTGGTCGCCCGGCCTCA
GTGAGCGAGCGAGCGCGCAGAGAGGGAGTGGCCAACTCCATCACTAGGGGTTCCTGCGGCCGCA
CGCGTGACATTGATTATTGACTAGTTATTAATAGTAATCAATTACGGGGTCATTAGTTCATAGC
CCATATATGGAGTTCCGCGTTACATAACTTACGGTAAATGGCCCGCCTGGCTGACCGCCCAACG
ACCCCCGCCCATTGACGTCAATAATGACGTATGTTCCCATAGTAACGCCAATAGGGACTTTCCA
TTGACGTCAATGGGTGGACTATTTACGGTAAACTGCCCACTTGGCAGTACATCAAGTGTATCAT
ATGCCAAGTACGCCCCCTATTGACGTCAATGACGGTAAATGGCCCGCCTGGCATTATGCCCAGT
ACATGACCTTATGGGACTTTCCTACTTGGCAGTACATCTACGTATTAGTCATCGCTATTACCAT
GGGTCGAGGTGAGCCCCACGTTCTGCTTCACTCTCCCCATCTCCCCCCCCTCCCCACCCCCAAT
TTTGTATTTATTTATTTTTTAATTATTTTGTGCAGCGATGGGGGCGGGGGGGGGGGGGGCGCGC
GCCAGGCGGGGCGGGGCGGGGCGAGGGGCGGGGCGGGGCGAGGCGGAGAGGTGCGGCGGCAGCC
AATCAGAGCGGCGCGCTCCGAAAGTTTCCTTTTATGGCGAGGCGGCGGCGGCGGCGGCCCTATA
AAAAGCGAAGCGCGCGGCGGGCGGGAGTCGCTGCGTTGCCTTCGCCCCGTGCCCCGCTCCGCGC
CGCCTCGCGCCGCCCGCCCCGGCTCTGACTGACCGCGTTACTCCCACAGGTGAGCGGGCGGGAC
GGCCCTTCTCCTCCGGGCTGTAATTAGCGCTTGGTTTAATGACGGCTCGTTTCTTTTCTGTGGC
TGCGTGAAAGCCTTAAAGGGCTCCGGGAGGGCCCTTTGTGCGGGGGGGAGCGGCTCGGGGGGTG
CGTGCGTGTGTGTGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGCGGCCGGGGGCGG
TGCCCCGCGGTGCGGGGGGGCTGCGAGGGGAACAAAGGCTGCGTGCGGGGTGTGTGCGTGGGGG
GGTGAGCAGGGGGTGTGGGCGCGGCGGTCGGGCTGTAACCCCCCCCTGCACCCCCCTCCCCGAG
TTGCTGAGCACGGCCCGGCTTCGGGTGCGGGGCTCCGTGCGGGGCGTGGCGCGGGGCTCGCCGT
GCCGGGCGGGGGGTGGCGGCAGGTGGGGGTGCCGGGCGGGGCGGGGCCGCCTCGGGCCGGGGAG
GGCTCGGGGGAGGGGCGCGGCGGCCCCCGGAGCGCCGGCGGTGTCCCCAGAGAGCCCGCACCAG
TGCCATCTGCTTGGCCGATTTGAGTCTCAGAGATGCCCggCTTGTGTCTCTCCGCTCTGAGCTG
TCTGTCTCGAGGCTCATATGGGAAAAGCAGGACCCGCAGCTGCGTCCGCCTCCCCTGCATTCTA
GAGCCTCTGCTAACCATGTTCATGCCTTCTTCTTTTTCCTACAGCTCCTGGGCAACGTGCTGGT
TATTGTGACCGGTCGCTAGCCACCATGGTGAGCAAGGGCGAGGAGCTGTTCACCGGGGTGGTGC
CCATCCTGGTCGAGCTGGACGGCGACGTAAACGGCCACAAGTTCAGCGTGTCCGGCGAGGGCGA
GGGCGATGCCACCTACGGCAAGCTGACCCTGAAGTTCATCTGCACCACCGGCAAGCTGCCCGTG
CCCTGGCCCACCCTCGTGACCACCCTGACCTACGGCGTGCAGTGCTTCAGCCGCTACCCCGACC
ACATGAAGCAGCACGACTTCTTCAAGTCCGCCATGCCCGAAGGCTACGTCCAGGAGCGCACCAT
CTTCTTCAAGGACGACGGCAACTACAAGACCCGCGCCGAGGTGAAGTTCGAGGGCGACACCCTG
GTGAACCGCATCGAGCTGAAGGGCATCGACTTCAAGGAGGACGGCAACATCCTGGGGCACAAGC
TGGAGTACAACTACAACAGCCACAACGTCTATATCATGGCCGACAAGCAGAAGAACGGCATCAA
GGTGAACTTCAAGATCCGCCACAACATCGAGGACGGCAGCGTGCAGCTCGCCGACCACTACCAG
CAGAACACCCCCATCGGCGACGGCCCCGTGCTGCTGCCCGACAACCACTACCTGAGCACCCAGT
CCGCCCTGAGCAAAGACCCCAACGAGAAGCGCGATCACATGGTCCTGCTGGAGTTCGTGACCGC
CGCCGGGATCACTCTCGGCATGGACGAGCTGTACAAGTAAcctgcaggcgccggcgGTGTCCCC
AGAGAGCCCGCACCAGTGCCATCTGCTTGGCCGATTTCTCGAAGTGCTTCTGCCGGCTTGTGTC
TCTCCGCTCTGAGCTGTGAGGAGCTTTCGGGATATGGGAAAAGCAGGACCCGCAGCTGCGTCCG
CCTCCCCTGCATGAGCTCGCTGATCAGCCTCGACTGTGCCTTCTAGTTGCCAGCCATCTGTTGT
TTGCCCCTCCCCCGTGCCTTCCTTGACCCTGGAAGGTGCCACTCCCACTGTCCTTTCCTAATAA
AATGAGGAAATTGCATCGCATTGTCTGAGTAGGTGTCATTCTATTCTGGGGGGTGGGGTGGGGC
AGGACAGCAAGGGGGAGGATTGGGAAGACAATAGCAGGCATGCTGGGGATGCGGTGGGCTCTAT
GGAAGCTTGAATTCAGCTGACGTGCCTCGGACCGCTAGGAACCCCTAGTGATGGAGTTGGCCAC
TCCCTCTCTGCGCGCTCGCTCGCTCACTGAGGCCGGGCGACCAAAGGTCGCCCGACGCCCGGGC
TTTGCCCGGGCGGCCTCAGTGAGCGAGCGAGCGCGCAG
Exemplary pITR.CAG.miR2i_miR5u-16_EGFP Construct sequence
(SEQ ID NO: 178)
CTGCGCGCTCGCTCGCTCACTGAGGCCGCCCGGGCGTCGGGCGACCTTTGGTCGCCCGGCCTCA
GTGAGCGAGCGAGCGCGCAGAGAGGGAGTGGCCAACTCCATCACTAGGGGTTCCTGCGGCCGCA
CGCGTGACATTGATTATTGACTAGTTATTAATAGTAATCAATTACGGGGTCATTAGTTCATAGC
CCATATATGGAGTTCCGCGTTACATAACTTACGGTAAATGGCCCGCCTGGCTGACCGCCCAACG
ACCCCCGCCCATTGACGTCAATAATGACGTATGTTCCCATAGTAACGCCAATAGGGACTTTCCA
TTGACGTCAATGGGTGGACTATTTACGGTAAACTGCCCACTTGGCAGTACATCAAGTGTATCAT
ATGCCAAGTACGCCCCCTATTGACGTCAATGACGGTAAATGGCCCGCCTGGCATTATGCCCAGT
ACATGACCTTATGGGACTTTCCTACTTGGCAGTACATCTACGTATTAGTCATCGCTATTACCAT
GGGTCGAGGTGAGCCCCACGTTCTGCTTCACTCTCCCCATCTCCCCCCCCTCCCCACCCCCAAT
TTTGTATTTATTTATTTTTTAATTATTTTGTGCAGCGATGGGGGCGGGGGGGGGGGGGGCGCGC
GCCAGGCGGGGCGGGGCGGGGCGAGGGGCGGGGCGGGGCGAGGCGGAGAGGTGCGGCGGCAGCC
AATCAGAGCGGCGCGCTCCGAAAGTTTCCTTTTATGGCGAGGCGGCGGCGGCGGCGGCCCTATA
AAAAGCGAAGCGCGCGGCGGGCGGGAGTCGCTGCGTTGCCTTCGCCCCGTGCCCCGCTCCGCGC
CGCCTCGCGCCGCCCGCCCCGGCTCTGACTGACCGCGTTACTCCCACAGGTGAGCGGGCGGGAC
GGCCCTTCTCCTCCGGGCTGTAATTAGCGCTTGGTTTAATGACGGCTCGTTTCTTTTCTGTGGC
TGCGTGAAAGCCTTAAAGGGCTCCGGGAGGGCCCTTTGTGCGGGGGGGAGCGGCTCGGGGGGTG
CGTGCGTGTGTGTGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGCGGCCGGGGGCGG
TGCCCCGCGGTGCGGGGGGGCTGCGAGGGGAACAAAGGCTGCGTGCGGGGTGTGTGCGTGGGGG
GGTGAGCAGGGGGTGTGGGCGCGGCGGTCGGGCTGTAACCCCCCCCTGCACCCCCCTCCCCGAG
TTGCTGAGCACGGCCCGGCTTCGGGTGCGGGGCTCCGTGCGGGGCGTGGCGCGGGGCTCGCCGT
GCCGGGCGGGGGGTGGCGGCAGGTGGGGGTGCCGGGCGGGGCGGGGCCGCCTCGGGCCGGGGAG
GGCTCGGGGGAGGGGCGCGGCGGCCCCCGGAGCGCCGGCGCATAGCTCTTATGATAGCAATGTC
AGCAGTGCCTTTGAGTCTCAGAGATGCCCGGGTTAAGATTCTAAAATTATCACGGGTATTACTT
GAGGCTCAGAAGTAAGGTTGACCATACTCTACAGTTGTGTCTAGAGCCTCTGCTAACCATGTTC
ATGCCTTCTTCTTTTTCCTACAGCTCCTGGGCAACGTGCTGGTTATTGTGACCGGTCGCTAGCC
ACCATGGTGAGCAAGGGCGAGGAGCTGTTCACCGGGGTGGTGCCCATCCTGGTCGAGCTGGACG
GCGACGTAAACGGCCACAAGTTCAGCGTGTCCGGCGAGGGCGAGGGCGATGCCACCTACGGCAA
GCTGACCCTGAAGTTCATCTGCACCACCGGCAAGCTGCCCGTGCCCTGGCCCACCCTCGTGACC
ACCCTGACCTACGGCGTGCAGTGCTTCAGCCGCTACCCCGACCACATGAAGCAGCACGACTTCT
TCAAGTCCGCCATGCCCGAAGGCTACGTCCAGGAGCGCACCATCTTCTTCAAGGACGACGGCAA
CTACAAGACCCGCGCCGAGGTGAAGTTCGAGGGCGACACCCTGGTGAACCGCATCGAGCTGAAG
GGCATCGACTTCAAGGAGGACGGCAACATCCTGGGGCACAAGCTGGAGTACAACTACAACAGCC
ACAACGTCTATATCATGGCCGACAAGCAGAAGAACGGCATCAAGGTGAACTTCAAGATCCGCCA
CAACATCGAGGACGGCAGCGTGCAGCTCGCCGACCACTACCAGCAGAACACCCCCATCGGCGAC
GGCCCCGTGCTGCTGCCCGACAACCACTACCTGAGCACCCAGTCCGCCCTGAGCAAAGACCCCA
ACGAGAAGCGCGATCACATGGTCCTGCTGGAGTTCGTGACCGCCGCCGGGATCACTCTCGGCAT
GGACGAGCTGTACAAGTAAcctgcaggcgccggcgCATAGCTCTTATGATAGCAATGTCAGCAG
TGCCTTTCTCCACCTTGACCACGCGTGTTAAGATTCTAAAATTATCTCGTGTGGTAAGTGGTGG
AGAGAAGTAAGGTTGACCATACTCTACAGTTGTGGAGCTCGCTGATCAGCCTCGACTGTGCCTT
CTAGTTGCCAGCCATCTGTTGTTTGCCCCTCCCCCGTGCCTTCCTTGACCCTGGAAGGTGCCAC
TCCCACTGTCCTTTCCTAATAAAATGAGGAAATTGCATCGCATTGTCTGAGTAGGTGTCATTCT
ATTCTGGGGGGTGGGGTGGGGCAGGACAGCAAGGGGGAGGATTGGGAAGACAATAGCAGGCATG
CTGGGGATGCGGTGGGCTCTATGGAAGCTTGAATTCAGCTGACGTGCCTCGGACCGCTAGGAAC
CCCTAGTGATGGAGTTGGCCACTCCCTCTCTGCGCGCTCGCTCGCTCACTGAGGCCGGGCGACC
AAAGGTCGCCCGACGCCCGGGCTTTGCCCGGGCGGCCTCAGTGAGCGAGCGAGCGCGCAG
Exemplary pITR.CAG.miR2i_miR5u-26_EGFP Construct sequence
(SEQ ID NO: 179)
CTGCGCGCTCGCTCGCTCACTGAGGCCGCCCGGGCGTCGGGCGACCTTTGGTCGCCCGGCCTCA
GTGAGCGAGCGAGCGCGCAGAGAGGGAGTGGCCAACTCCATCACTAGGGGTTCCTGCGGCCGCA
CGCGTGACATTGATTATTGACTAGTTATTAATAGTAATCAATTACGGGGTCATTAGTTCATAGC
CCATATATGGAGTTCCGCGTTACATAACTTACGGTAAATGGCCCGCCTGGCTGACCGCCCAACG
ACCCCCGCCCATTGACGTCAATAATGACGTATGTTCCCATAGTAACGCCAATAGGGACTTTCCA
TTGACGTCAATGGGTGGACTATTTACGGTAAACTGCCCACTTGGCAGTACATCAAGTGTATCAT
ATGCCAAGTACGCCCCCTATTGACGTCAATGACGGTAAATGGCCCGCCTGGCATTATGCCCAGT
ACATGACCTTATGGGACTTTCCTACTTGGCAGTACATCTACGTATTAGTCATCGCTATTACCAT
GGGTCGAGGTGAGCCCCACGTTCTGCTTCACTCTCCCCATCTCCCCCCCCTCCCCACCCCCAAT
TTTGTATTTATTTATTTTTTAATTATTTTGTGCAGCGATGGGGGCGGGGGGGGGGGGGGCGCGC
GCCAGGCGGGGCGGGGCGGGGCGAGGGGCGGGGCGGGGCGAGGCGGAGAGGTGCGGCGGCAGCC
AATCAGAGCGGCGCGCTCCGAAAGTTTCCTTTTATGGCGAGGCGGCGGCGGCGGCGGCCCTATA
AAAAGCGAAGCGCGCGGCGGGCGGGAGTCGCTGCGTTGCCTTCGCCCCGTGCCCCGCTCCGCGC
CGCCTCGCGCCGCCCGCCCCGGCTCTGACTGACCGCGTTACTCCCACAGGTGAGCGGGCGGGAC
GGCCCTTCTCCTCCGGGCTGTAATTAGCGCTTGGTTTAATGACGGCTCGTTTCTTTTCTGTGGC
TGCGTGAAAGCCTTAAAGGGCTCCGGGAGGGCCCTTTGTGCGGGGGGGAGCGGCTCGGGGGGTG
CGTGCGTGTGTGTGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGCGGCCGGGGGCGG
TGCCCCGCGGTGCGGGGGGGCTGCGAGGGGAACAAAGGCTGCGTGCGGGGTGTGTGCGTGGGGG
GGTGAGCAGGGGGTGTGGGCGCGGCGGTCGGGCTGTAACCCCCCCCTGCACCCCCCTCCCCGAG
TTGCTGAGCACGGCCCGGCTTCGGGTGCGGGGCTCCGTGCGGGGCGTGGCGCGGGGCTCGCCGT
GCCGGGCGGGGGGTGGCGGCAGGTGGGGGTGCCGGGCGGGGCGGGGCCGCCTCGGGCCGGGGAG
GGCTCGGGGGAGGGGCGCGGCGGCCCCCGGAGCGCCGGCGGAGCAGGAGGACCAAGGCCCTGGC
GAAGGCCGTGGCCTCGTTGAGTCTCAGAGATGCCCGGTGTGCAGGTCCCAATGGCCGGGTATTA
CTGAGATTCCACGGGGACGCGGGCCTGGACGCCGGCATCCGGGCTCAGGACCCCCCTCTCTAGA
GCCTCTGCTAACCATGTTCATGCCTTCTTCTTTTTCCTACAGCTCCTGGGCAACGTGCTGGTTA
TTGTGACCGGTCGCTAGCCACCATGGTGAGCAAGGGCGAGGAGCTGTTCACCGGGGTGGTGCCC
ATCCTGGTCGAGCTGGACGGCGACGTAAACGGCCACAAGTTCAGCGTGTCCGGCGAGGGCGAGG
GCGATGCCACCTACGGCAAGCTGACCCTGAAGTTCATCTGCACCACCGGCAAGCTGCCCGTGCC
CTGGCCCACCCTCGTGACCACCCTGACCTACGGCGTGCAGTGCTTCAGCCGCTACCCCGACCAC
ATGAAGCAGCACGACTTCTTCAAGTCCGCCATGCCCGAAGGCTACGTCCAGGAGCGCACCATCT
TCTTCAAGGACGACGGCAACTACAAGACCCGCGCCGAGGTGAAGTTCGAGGGCGACACCCTGGT
GAACCGCATCGAGCTGAAGGGCATCGACTTCAAGGAGGACGGCAACATCCTGGGGCACAAGCTG
GAGTACAACTACAACAGCCACAACGTCTATATCATGGCCGACAAGCAGAAGAACGGCATCAAGG
TGAACTTCAAGATCCGCCACAACATCGAGGACGGCAGCGTGCAGCTCGCCGACCACTACCAGCA
GAACACCCCCATCGGCGACGGCCCCGTGCTGCTGCCCGACAACCACTACCTGAGCACCCAGTCC
GCCCTGAGCAAAGACCCCAACGAGAAGCGCGATCACATGGTCCTGCTGGAGTTCGTGACCGCCG
CCGGGATCACTCTCGGCATGGACGAGCTGTACAAGTAAcctgcaggcgccggcgGAGCAGGAGG
ACCAAGGCCCTGGCGAAGGCCGTGGCCTCGTTCTCCACCTTGACCACGCGTTGTGCAGGTCCCA
ATGGGCGTGTGGTAGAGGTGGAGCACGGGGACGCGGGCCTGGACGCCGGCATCCGGGCTCAGGA
CCCCCCTCGAGCTCGCTGATCAGCCTCGACTGTGCCTTCTAGTTGCCAGCCATCTGTTGTTTGC
CCCTCCCCCGTGCCTTCCTTGACCCTGGAAGGTGCCACTCCCACTGTCCTTTCCTAATAAAATG
AGGAAATTGCATCGCATTGTCTGAGTAGGTGTCATTCTATTCTGGGGGGTGGGGTGGGGCAGGA
CAGCAAGGGGGAGGATTGGGAAGACAATAGCAGGCATGCTGGGGATGCGGTGGGCTCTATGGAA
GCTTGAATTCAGCTGACGTGCCTCGGACCGCTAGGAACCCCTAGTGATGGAGTTGGCCACTCCC
TCTCTGCGCGCTCGCTCGCTCACTGAGGCCGGGCGACCAAAGGTCGCCCGACGCCCGGGCTTTG
CCCGGGCGGCCTCAGTGAGCGAGCGAGCGCGCAG
Exemplary pITR.CAG.miR2i_miR5u-96_EGFP Construct sequence
(SEQ ID NO: 180)
CTGCGCGCTCGCTCGCTCACTGAGGCCGCCCGGGCGTCGGGCGACCTTTGGTCGCCCGGCCTCA
GTGAGCGAGCGAGCGCGCAGAGAGGGAGTGGCCAACTCCATCACTAGGGGTTCCTGCGGCCGCA
CGCGTGACATTGATTATTGACTAGTTATTAATAGTAATCAATTACGGGGTCATTAGTTCATAGC
CCATATATGGAGTTCCGCGTTACATAACTTACGGTAAATGGCCCGCCTGGCTGACCGCCCAACG
ACCCCCGCCCATTGACGTCAATAATGACGTATGTTCCCATAGTAACGCCAATAGGGACTTTCCA
TTGACGTCAATGGGTGGACTATTTACGGTAAACTGCCCACTTGGCAGTACATCAAGTGTATCAT
ATGCCAAGTACGCCCCCTATTGACGTCAATGACGGTAAATGGCCCGCCTGGCATTATGCCCAGT
ACATGACCTTATGGGACTTTCCTACTTGGCAGTACATCTACGTATTAGTCATCGCTATTACCAT
GGGTCGAGGTGAGCCCCACGTTCTGCTTCACTCTCCCCATCTCCCCCCCCTCCCCACCCCCAAT
TTTGTATTTATTTATTTTTTAATTATTTTGTGCAGCGATGGGGGCGGGGGGGGGGGGGGCGCGC
GCCAGGCGGGGCGGGGCGGGGCGAGGGGCGGGGCGGGGCGAGGCGGAGAGGTGCGGCGGCAGCC
AATCAGAGCGGCGCGCTCCGAAAGTTTCCTTTTATGGCGAGGCGGCGGCGGCGGCGGCCCTATA
AAAAGCGAAGCGCGCGGCGGGCGGGAGTCGCTGCGTTGCCTTCGCCCCGTGCCCCGCTCCGCGC
CGCCTCGCGCCGCCCGCCCCGGCTCTGACTGACCGCGTTACTCCCACAGGTGAGCGGGCGGGAC
GGCCCTTCTCCTCCGGGCTGTAATTAGCGCTTGGTTTAATGACGGCTCGTTTCTTTTCTGTGGC
TGCGTGAAAGCCTTAAAGGGCTCCGGGAGGGCCCTTTGTGCGGGGGGGAGCGGCTCGGGGGGTG
CGTGCGTGTGTGTGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGCGGCCGGGGGCGG
TGCCCCGCGGTGCGGGGGGGCTGCGAGGGGAACAAAGGCTGCGTGCGGGGTGTGTGCGTGGGGG
GGTGAGCAGGGGGTGTGGGCGCGGCGGTCGGGCTGTAACCCCCCCCTGCACCCCCCTCCCCGAG
TTGCTGAGCACGGCCCGGCTTCGGGTGCGGGGCTCCGTGCGGGGCGTGGCGCGGGGCTCGCCGT
GCCGGGCGGGGGGTGGCGGCAGGTGGGGGTGCCGGGCGGGGCGGGGCCGCCTCGGGCCGGGGAG
GGCTCGGGGGAGGGGCGCGGCGGCCCCCGGAGCGCCGGCGGTGTCCCCAGAGAGCCCGCACCAG
TGCCATCTGCTTGGCCGATTTGAGTCTCAGAGATGCCCggCTTGTGTCTCTCCGCTCTGAGCTG
TCTGTCTCGAGGCTCATATGGGAAAAGCAGGACCCGCAGCTGCGTCCGCCTCCCCTGCATTCTA
GAGCCTCTGCTAACCATGTTCATGCCTTCTTCTTTTTCCTACAGCTCCTGGGCAACGTGCTGGT
TATTGTGACCGGTCGCTAGCCACCATGGTGAGCAAGGGCGAGGAGCTGTTCACCGGGGTGGTGC
CCATCCTGGTCGAGCTGGACGGCGACGTAAACGGCCACAAGTTCAGCGTGTCCGGCGAGGGCGA
GGGCGATGCCACCTACGGCAAGCTGACCCTGAAGTTCATCTGCACCACCGGCAAGCTGCCCGTG
CCCTGGCCCACCCTCGTGACCACCCTGACCTACGGCGTGCAGTGCTTCAGCCGCTACCCCGACC
ACATGAAGCAGCACGACTTCTTCAAGTCCGCCATGCCCGAAGGCTACGTCCAGGAGCGCACCAT
qTTCTTCAAGGACGACGGCAACTACAAGACCCGCGCCGAGGTGAAGTTCGAGGGCGACACCCTG
GTGAACCGCATCGAGCTGAAGGGCATCGACTTCAAGGAGGACGGCAACATCCTGGGGCACAAGC
TGGAGTACAACTACAACAGCCACAACGTCTATATCATGGCCGACAAGCAGAAGAACGGCATCAA
GGTGAACTTCAAGATCCGCCACAACATCGAGGACGGCAGCGTGCAGCTCGCCGACCACTACCAG
CAGAACACCCCCATCGGCGACGGCCCCGTGCTGCTGCCCGACAACCACTACCTGAGCACCCAGT
CCGCCCTGAGCAAAGACCCCAACGAGAAGCGCGATCACATGGTCCTGCTGGAGTTCGTGACCGC
CGCCGGGATCACTCTCGGCATGGACGAGCTGTACAAGTAAcctgcaggcgccggcgGTGTCCCC
AGAGAGCCCGCACCAGTGCCATCTGCTTGGCCGATTTCTCCACCTTGACCACGCGTCTTGTGTC
TCTCCGCTCTGAGGTGACTGGTTGGGTGGAGATATGGGAAAAGCAGGACCCGCAGCTGCGTCCG
CCTCCCCTGCATGAGCTCGCTGATCAGCCTCGACTGTGCCTTCTAGTTGCCAGCCATCTGTTGT
TTGCCCCTCCCCCGTGCCTTCCTTGACCCTGGAAGGTGCCACTCCCACTGTCCTTTCCTAATAA
AATGAGGAAATTGCATCGCATTGTCTGAGTAGGTGTCATTCTATTCTGGGGGGTGGGGTGGGGC
AGGACAGCAAGGGGGAGGATTGGGAAGACAATAGCAGGCATGCTGGGGATGCGGTGGGCTCTAT
GGAAGCTTGAATTCAGCTGACGTGCCTCGGACCGCTAGGAACCCCTAGTGATGGAGTTGGCCAC
TCCCTCTCTGCGCGCTCGCTCGCTCACTGAGGCCGGGCGACCAAAGGTCGCCCGACGCCCGGGC
TTTGCCCGGGCGGCCTCAGTGAGCGAGCGAGCGCGCAG
Exemplary pITR.CAG.miR2i_miR6u-16_EGFP Construct sequence
(SEQ ID NO: 181)
CTGCGCGCTCGCTCGCTCACTGAGGCCGCCCGGGCGTCGGGCGACCTTTGGTCGCCCGGCCTCA
GTGAGCGAGCGAGCGCGCAGAGAGGGAGTGGCCAACTCCATCACTAGGGGTTCCTGCGGCCGCA
CGCGTGACATTGATTATTGACTAGTTATTAATAGTAATCAATTACGGGGTCATTAGTTCATAGC
CCATATATGGAGTTCCGCGTTACATAACTTACGGTAAATGGCCCGCCTGGCTGACCGCCCAACG
ACCCCCGCCCATTGACGTCAATAATGACGTATGTTCCCATAGTAACGCCAATAGGGACTTTCCA
TTGACGTCAATGGGTGGACTATTTACGGTAAACTGCCCACTTGGCAGTACATCAAGTGTATCAT
ATGCCAAGTACGCCCCCTATTGACGTCAATGACGGTAAATGGCCCGCCTGGCATTATGCCCAGT
ACATGACCTTATGGGACTTTCCTACTTGGCAGTACATCTACGTATTAGTCATCGCTATTACCAT
GGGTCGAGGTGAGCCCCACGTTCTGCTTCACTCTCCCCATCTCCCCCCCCTCCCCACCCCCAAT
TTTGTATTTATTTATTTTTTAATTATTTTGTGCAGCGATGGGGGCGGGGGGGGGGGGGGCGCGC
GCCAGGCGGGGCGGGGCGGGGCGAGGGGCGGGGCGGGGCGAGGCGGAGAGGTGCGGCGGCAGCC
AATCAGAGCGGCGCGCTCCGAAAGTTTCCTTTTATGGCGAGGCGGCGGCGGCGGCGGCCCTATA
AAAAGCGAAGCGCGCGGCGGGCGGGAGTCGCTGCGTTGCCTTCGCCCCGTGCCCCGCTCCGCGC
CGCCTCGCGCCGCCCGCCCCGGCTCTGACTGACCGCGTTACTCCCACAGGTGAGCGGGCGGGAC
GGCCCTTCTCCTCCGGGCTGTAATTAGCGCTTGGTTTAATGACGGCTCGTTTCTTTTCTGTGGC
TGCGTGAAAGCCTTAAAGGGCTCCGGGAGGGCCCTTTGTGCGGGGGGGAGCGGCTCGGGGGGTG
CGTGCGTGTGTGTGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGCGGCCGGGGGCGG
TGCCCCGCGGTGCGGGGGGGCTGCGAGGGGAACAAAGGCTGCGTGCGGGGTGTGTGCGTGGGGG
GGTGAGCAGGGGGTGTGGGCGCGGCGGTCGGGCTGTAACCCCCCCCTGCACCCCCCTCCCCGAG
TTGCTGAGCACGGCCCGGCTTCGGGTGCGGGGCTCCGTGCGGGGCGTGGCGCGGGGCTCGCCGT
GCCGGGCGGGGGGTGGCGGCAGGTGGGGGTGCCGGGCGGGGCGGGGCCGCCTCGGGCCGGGGAG
GGCTCGGGGGAGGGGCGCGGCGGCCCCCGGAGCGCCGGCGCATAGCTCTTATGATAGCAATGTC
AGCAGTGCCTTTGAGTCTCAGAGATGCCCGGGTTAAGATTCTAAAATTATCACGGGTATTACTT
GAGGCTCAGAAGTAAGGTTGACCATACTCTACAGTTGTGTCTAGAGCCTCTGCTAACCATGTTC
ATGCCTTCTTCTTTTTCCTACAGCTCCTGGGCAACGTGCTGGTTATTGTGACCGGTCGCTAGCC
ACCATGGTGAGCAAGGGCGAGGAGCTGTTCACCGGGGTGGTGCCCATCCTGGTCGAGCTGGACG
GCGACGTAAACGGCCACAAGTTCAGCGTGTCCGGCGAGGGCGAGGGCGATGCCACCTACGGCAA
GCTGACCCTGAAGTTCATCTGCACCACCGGCAAGCTGCCCGTGCCCTGGCCCACCCTCGTGACC
ACCCTGACCTACGGCGTGCAGTGCTTCAGCCGCTACCCCGACCACATGAAGCAGCACGACTTCT
TCAAGTCCGCCATGCCCGAAGGCTACGTCCAGGAGCGCACCATCTTCTTCAAGGACGACGGCAA
CTACAAGACCCGCGCCGAGGTGAAGTTCGAGGGCGACACCCTGGTGAACCGCATCGAGCTGAAG
GGCATCGACTTCAAGGAGGACGGCAACATCCTGGGGCACAAGCTGGAGTACAACTACAACAGCC
ACAACGTCTATATCATGGCCGACAAGCAGAAGAACGGCATCAAGGTGAACTTCAAGATCCGCCA
CAACATCGAGGACGGCAGCGTGCAGCTCGCCGACCACTACCAGCAGAACACCCCCATCGGCGAC
GGCCCCGTGCTGCTGCCCGACAACCACTACCTGAGCACCCAGTCCGCCCTGAGCAAAGACCCCA
ACGAGAAGCGCGATCACATGGTCCTGCTGGAGTTCGTGACCGCCGCCGGGATCACTCTCGGCAT
GGACGAGCTGTACAAGTAAcctgcaggcgccggcgCATAGCTCTTATGATAGCAATGTCAGCAG
TGCCTATACTGTCATAGTAGTACCAGGTTAAGATTCTAAAATTATCATGGTACTAATGTTGGCA
GTGTAAGTAAGGTTGACCATACTCTACAGTTGTGGAGCTCGCTGATCAGCCTCGACTGTGCCTT
CTAGTTGCCAGCCATCTGTTGTTTGCCCCTCCCCCGTGCCTTCCTTGACCCTGGAAGGTGCCAC
TCCCACTGTCCTTTCCTAATAAAATGAGGAAATTGCATCGCATTGTCTGAGTAGGTGTCATTCT
ATTCTGGGGGGTGGGGTGGGGCAGGACAGCAAGGGGGAGGATTGGGAAGACAATAGCAGGCATG
CTGGGGATGCGGTGGGCTCTATGGAAGCTTGAATTCAGCTGACGTGCCTCGGACCGCTAGGAAC
CCCTAGTGATGGAGTTGGCCACTCCCTCTCTGCGCGCTCGCTCGCTCACTGAGGCCGGGCGACC
AAAGGTCGCCCGACGCCCGGGCTTTGCCCGGGCGGCCTCAGTGAGCGAGCGAGCGCGCAG
Exemplary pITR.CAG.miR2i_miR6u-26_EGFP Construct sequence
(SEQ ID NO: 182)
CTGCGCGCTCGCTCGCTCACTGAGGCCGCCCGGGCGTCGGGCGACCTTTGGTCGCCCGGCCTCA
GTGAGCGAGCGAGCGCGCAGAGAGGGAGTGGCCAACTCCATCACTAGGGGTTCCTGCGGCCGCA
CGCGTGACATTGATTATTGACTAGTTATTAATAGTAATCAATTACGGGGTCATTAGTTCATAGC
CCATATATGGAGTTCCGCGTTACATAACTTACGGTAAATGGCCCGCCTGGCTGACCGCCCAACG
ACCCCCGCCCATTGACGTCAATAATGACGTATGTTCCCATAGTAACGCCAATAGGGACTTTCCA
TTGACGTCAATGGGTGGACTATTTACGGTAAACTGCCCACTTGGCAGTACATCAAGTGTATCAT
ATGCCAAGTACGCCCCCTATTGACGTCAATGACGGTAAATGGCCCGCCTGGCATTATGCCCAGT
ACATGACCTTATGGGACTTTCCTACTTGGCAGTACATCTACGTATTAGTCATCGCTATTACCAT
GGGTCGAGGTGAGCCCCACGTTCTGCTTCACTCTCCCCATCTCCCCCCCCTCCCCACCCCCAAT
TTTGTATTTATTTATTTTTTAATTATTTTGTGCAGCGATGGGGGCGGGGGGGGGGGGGGCGCGC
GCCAGGCGGGGCGGGGCGGGGCGAGGGGCGGGGCGGGGCGAGGCGGAGAGGTGCGGCGGCAGCC
AATCAGAGCGGCGCGCTCCGAAAGTTTCCTTTTATGGCGAGGCGGCGGCGGCGGCGGCCCTATA
AAAAGCGAAGCGCGCGGCGGGCGGGAGTCGCTGCGTTGCCTTCGCCCCGTGCCCCGCTCCGCGC
CGCCTCGCGCCGCCCGCCCCGGCTCTGACTGACCGCGTTACTCCCACAGGTGAGCGGGCGGGAC
GGCCCTTCTCCTCCGGGCTGTAATTAGCGCTTGGTTTAATGACGGCTCGTTTCTTTTCTGTGGC
TGCGTGAAAGCCTTAAAGGGCTCCGGGAGGGCCCTTTGTGCGGGGGGGAGCGGCTCGGGGGGTG
CGTGCGTGTGTGTGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGCGGCCGGGGGCGG
TGCCCCGCGGTGCGGGGGGGCTGCGAGGGGAACAAAGGCTGCGTGCGGGGTGTGTGCGTGGGGG
GGTGAGCAGGGGGTGTGGGCGCGGCGGTCGGGCTGTAACCCCCCCCTGCACCCCCCTCCCCGAG
TTGCTGAGCACGGCCCGGCTTCGGGTGCGGGGCTCCGTGCGGGGCGTGGCGCGGGGCTCGCCGT
GCCGGGCGGGGGGTGGCGGCAGGTGGGGGTGCCGGGCGGGGCGGGGCCGCCTCGGGCCGGGGAG
GGCTCGGGGGAGGGGCGCGGCGGCCCCCGGAGCGCCGGCGGAGCAGGAGGACCAAGGCCCTGGC
GAAGGCCGTGGCCTCGTTGAGTCTCAGAGATGCCCGGTGTGCAGGTCCCAATGGCCGGGTATTA
CTGAGATTCCACGGGGACGCGGGCCTGGACGCCGGCATCCGGGCTCAGGACCCCCCTCTCTAGA
GCCTCTGCTAACCATGTTCATGCCTTCTTCTTTTTCCTACAGCTCCTGGGCAACGTGCTGGTTA
TTGTGACCGGTCGCTAGCCACCATGGTGAGCAAGGGCGAGGAGCTGTTCACCGGGGTGGTGCCC
ATCCTGGTCGAGCTGGACGGCGACGTAAACGGCCACAAGTTCAGCGTGTCCGGCGAGGGCGAGG
GCGATGCCACCTACGGCAAGCTGACCCTGAAGTTCATCTGCACCACCGGCAAGCTGCCCGTGCC
CTGGCCCACCCTCGTGACCACCCTGACCTACGGCGTGCAGTGCTTCAGCCGCTACCCCGACCAC
ATGAAGCAGCACGACTTCTTCAAGTCCGCCATGCCCGAAGGCTACGTCCAGGAGCGCACCATCT
TCTTCAAGGACGACGGCAACTACAAGACCCGCGCCGAGGTGAAGTTCGAGGGCGACACCCTGGT
GAACCGCATCGAGCTGAAGGGCATCGACTTCAAGGAGGACGGCAACATCCTGGGGCACAAGCTG
GAGTACAACTACAACAGCCACAACGTCTATATCATGGCCGACAAGCAGAAGAACGGCATCAAGG
TGAACTTCAAGATCCGCCACAACATCGAGGACGGCAGCGTGCAGCTCGCCGACCACTACCAGCA
GAACACCCCCATCGGCGACGGCCCCGTGCTGCTGCCCGACAACCACTACCTGAGCACCCAGTCC
GCCCTGAGCAAAGACCCCAACGAGAAGCGCGATCACATGGTCCTGCTGGAGTTCGTGACCGCCG
CCGGGATCACTCTCGGCATGGACGAGCTGTACAAGTAAcctgcaggcgccggcgGAGCAGGAGG
ACCAAGGCCCTGGCGAAGGCCGTGGCCTCGATACTGTCATAGTAGTACCAGTGTGCAGGTCCCA
ATGGCTGGTACTAATGTGACAGTCTCGGGGACGCGGGCCTGGACGCCGGCATCCGGGCTCAGGA
CCCCCCTCGAGCTCGCTGATCAGCCTCGACTGTGCCTTCTAGTTGCCAGCCATCTGTTGTTTGC
CCCTCCCCCGTGCCTTCCTTGACCCTGGAAGGTGCCACTCCCACTGTCCTTTCCTAATAAAATG
AGGAAATTGCATCGCATTGTCTGAGTAGGTGTCATTCTATTCTGGGGGGTGGGGTGGGGCAGGA
CAGCAAGGGGGAGGATTGGGAAGACAATAGCAGGCATGCTGGGGATGCGGTGGGCTCTATGGAA
GCTTGAATTCAGCTGACGTGCCTCGGACCGCTAGGAACCCCTAGTGATGGAGTTGGCCACTCCC
TCTCTGCGCGCTCGCTCGCTCACTGAGGCCGGGCGACCAAAGGTCGCCCGACGCCCGGGCTTTG
CCCGGGCGGCCTCAGTGAGCGAGCGAGCGCGCAG
Exemplary pITR.CAG.miR2i_miR6u-96_EGFP Construct sequence
(SEQ ID NO: 183)
CTGCGCGCTCGCTCGCTCACTGAGGCCGCCCGGGCGTCGGGCGACCTTTGGTCGCCCGGCCTCA
GTGAGCGAGCGAGCGCGCAGAGAGGGAGTGGCCAACTCCATCACTAGGGGTTCCTGCGGCCGCA
CGCGTGACATTGATTATTGACTAGTTATTAATAGTAATCAATTACGGGGTCATTAGTTCATAGC
CCATATATGGAGTTCCGCGTTACATAACTTACGGTAAATGGCCCGCCTGGCTGACCGCCCAACG
ACCCCCGCCCATTGACGTCAATAATGACGTATGTTCCCATAGTAACGCCAATAGGGACTTTCCA
TTGACGTCAATGGGTGGACTATTTACGGTAAACTGCCCACTTGGCAGTACATCAAGTGTATCAT
ATGCCAAGTACGCCCCCTATTGACGTCAATGACGGTAAATGGCCCGCCTGGCATTATGCCCAGT
ACATGACCTTATGGGACTTTCCTACTTGGCAGTACATCTACGTATTAGTCATCGCTATTACCAT
GGGTCGAGGTGAGCCCCACGTTCTGCTTCACTCTCCCCATCTCCCCCCCCTCCCCACCCCCAAT
TTTGTATTTATTTATTTTTTAATTATTTTGTGCAGCGATGGGGGCGGGGGGGGGGGGGGCGCGC
GCCAGGCGGGGCGGGGCGGGGCGAGGGGCGGGGCGGGGCGAGGCGGAGAGGTGCGGCGGCAGCC
AATCAGAGCGGCGCGCTCCGAAAGTTTCCTTTTATGGCGAGGCGGCGGCGGCGGCGGCCCTATA
AAAAGCGAAGCGCGCGGCGGGCGGGAGTCGCTGCGTTGCCTTCGCCCCGTGCCCCGCTCCGCGC
CGCCTCGCGCCGCCCGCCCCGGCTCTGACTGACCGCGTTACTCCCACAGGTGAGCGGGCGGGAC
GGCCCTTCTCCTCCGGGCTGTAATTAGCGCTTGGTTTAATGACGGCTCGTTTCTTTTCTGTGGC
TGCGTGAAAGCCTTAAAGGGCTCCGGGAGGGCCCTTTGTGCGGGGGGGAGCGGCTCGGGGGGTG
CGTGCGTGTGTGTGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGCGGCCGGGGGCGG
TGCCCCGCGGTGCGGGGGGGCTGCGAGGGGAACAAAGGCTGCGTGCGGGGTGTGTGCGTGGGGG
GGTGAGCAGGGGGTGTGGGCGCGGCGGTCGGGCTGTAACCCCCCCCTGCACCCCCCTCCCCGAG
TTGCTGAGCACGGCCCGGCTTCGGGTGCGGGGCTCCGTGCGGGGCGTGGCGCGGGGCTCGCCGT
GCCGGGCGGGGGGTGGCGGCAGGTGGGGGTGCCGGGCGGGGCGGGGCCGCCTCGGGCCGGGGAG
GGCTCGGGGGAGGGGCGCGGCGGCCCCCGGAGCGCCGGCGGTGTCCCCAGAGAGCCCGCACCAG
TGCCATCTGCTTGGCCGATTTGAGTCTCAGAGATGCCCggCTTGTGTCTCTCCGCTCTGAGCTG
TCTGTCTCGAGGCTCATATGGGAAAAGCAGGACCCGCAGCTGCGTCCGCCTCCCCTGCATTCTA
GAGCCTCTGCTAACCATGTTCATGCCTTCTTCTTTTTCCTACAGCTCCTGGGCAACGTGCTGGT
TATTGTGACCGGTCGCTAGCCACCATGGTGAGCAAGGGCGAGGAGCTGTTCACCGGGGTGGTGC
CCATCCTGGTCGAGCTGGACGGCGACGTAAACGGCCACAAGTTCAGCGTGTCCGGCGAGGGCGA
GGGCGATGCCACCTACGGCAAGCTGACCCTGAAGTTCATCTGCACCACCGGCAAGCTGCCCGTG
CCCTGGCCCACCCTCGTGACCACCCTGACCTACGGCGTGCAGTGCTTCAGCCGCTACCCCGACC
ACATGAAGCAGCACGACTTCTTCAAGTCCGCCATGCCCGAAGGCTACGTCCAGGAGCGCACCAT
CTTCTTCAAGGACGACGGCAACTACAAGACCCGCGCCGAGGTGAAGTTCGAGGGCGACACCCTG
GTGAACCGCATCGAGCTGAAGGGCATCGACTTCAAGGAGGACGGCAACATCCTGGGGCACAAGC
TGGAGTACAACTACAACAGCCACAACGTCTATATCATGGCCGACAAGCAGAAGAACGGCATCAA
GGTGAACTTCAAGATCCGCCACAACATCGAGGACGGCAGCGTGCAGCTCGCCGACCACTACCAG
CAGAACACCCCCATCGGCGACGGCCCCGTGCTGCTGCCCGACAACCACTACCTGAGCACCCAGT
CCGCCCTGAGCAAAGACCCCAACGAGAAGCGCGATCACATGGTCCTGCTGGAGTTCGTGACCGC
CGCCGGGATCACTCTCGGCATGGACGAGCTGTACAAGTAAcctgcaggcgccggcgGTGTCCCC
AGAGAGCCCGCACCAGTGCCATCTGCTTGGCCGATATACTGTCATAGTAGTACCAGCTTGTGTC
TCTCCGCTCTGAGCTGTCACTGCTTGACGGTAAATGGGAAAAGCAGGACCCGCAGCTGCGTCCG
CCTCCCCTGCATGAGCTCGCTGATCAGCCTCGACTGTGCCTTCTAGTTGCCAGCCATCTGTTGT
TTGCCCCTCCCCCGTGCCTTCCTTGACCCTGGAAGGTGCCACTCCCACTGTCCTTTCCTAATAA
AATGAGGAAATTGCATCGCATTGTCTGAGTAGGTGTCATTCTATTCTGGGGGGTGGGGTGGGGC
AGGACAGCAAGGGGGAGGATTGGGAAGACAATAGCAGGCATGCTGGGGATGCGGTGGGCTCTAT
GGAAGCTTGAATTCAGCTGACGTGCCTCGGACCGCTAGGAACCCCTAGTGATGGAGTTGGCCAC
TCCCTCTCTGCGCGCTCGCTCGCTCACTGAGGCCGGGCGACCAAAGGTCGCCCGACGCCCGGGC
TTTGCCCGGGCGGCCTCAGTGAGCGAGCGAGCGCGCAG
Exemplary pITR.CAG.miR4-16_EGFP Construct sequence
(SEQ ID NO: 184)
CTGCGCGCTCGCTCGCTCACTGAGGCCGCCCGGGCGTCGGGCGACCTTTGGTCGCCCGGCCTCA
GTGAGCGAGCGAGCGCGCAGAGAGGGAGTGGCCAACTCCATCACTAGGGGTTCCTGCGGCCGCA
CGCGTGACATTGATTATTGACTAGTTATTAATAGTAATCAATTACGGGGTCATTAGTTCATAGC
CCATATATGGAGTTCCGCGTTACATAACTTACGGTAAATGGCCCGCCTGGCTGACCGCCCAACG
ACCCCCGCCCATTGACGTCAATAATGACGTATGTTCCCATAGTAACGCCAATAGGGACTTTCCA
TTGACGTCAATGGGTGGACTATTTACGGTAAACTGCCCACTTGGCAGTACATCAAGTGTATCAT
ATGCCAAGTACGCCCCCTATTGACGTCAATGACGGTAAATGGCCCGCCTGGCATTATGCCCAGT
ACATGACCTTATGGGACTTTCCTACTTGGCAGTACATCTACGTATTAGTCATCGCTATTACCAT
GGGTCGAGGTGAGCCCCACGTTCTGCTTCACTCTCCCCATCTCCCCCCCCTCCCCACCCCCAAT
TTTGTATTTATTTATTTTTTAATTATTTTGTGCAGCGATGGGGGCGGGGGGGGGGGGGGCGCGC
GCCAGGCGGGGCGGGGCGGGGCGAGGGGCGGGGCGGGGCGAGGCGGAGAGGTGCGGCGGCAGCC
AATCAGAGCGGCGCGCTCCGAAAGTTTCCTTTTATGGCGAGGCGGCGGCGGCGGCGGCCCTATA
AAAAGCGAAGCGCGCGGCGGGCGGGAGTCGCTGCGTTGCCTTCGCCCCGTGCCCCGCTCCGCGC
CGCCTCGCGCCGCCCGCCCCGGCTCTGACTGACCGCGTTACTCCCACAGGTGAGCGGGCGGGAC
GGCCCTTCTCCTCCGGGCTGTAATTAGCGCTTGGTTTAATGACGGCTCGTTTCTTTTCTGTGGC
TGCGTGAAAGCCTTAAAGGGCTCCGGGAGGGCCCTTTGTGCGGGGGGGAGCGGCTCGGGGGGTG
CGTGCGTGTGTGTGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGCGGCCGGGGGCGG
TGCCCCGCGGTGCGGGGGGGCTGCGAGGGGAACAAAGGCTGCGTGCGGGGTGTGTGCGTGGGGG
GGTGAGCAGGGGGTGTGGGCGCGGCGGTCGGGCTGTAACCCCCCCCTGCACCCCCCTCCCCGAG
TTGCTGAGCACGGCCCGGCTTCGGGTGCGGGGCTCCGTGCGGGGCGTGGCGCGGGGCTCGCCGT
GCCGGGCGGGGGGTGGCGGCAGGTGGGGGTGCCGGGCGGGGCGGGGCCGCCTCGGGCCGGGGAG
GGCTCGGGGGAGGGGCGCGGCGGCCCCCGGAGCGCCGGCGCATAGCTCTTATGATAGCAATGTC
AGCAGTGCCTTTCTCGAAGTGCTTCTGCCGGGTTAAGATTCTAAAATTATCACGGTAGAAACAT
CTTCGGGAGAAGTAAGGTTGACCATACTCTACAGTTGTGTCTAGAGCCTCTGCTAACCATGTTC
ATGCCTTCTTCTTTTTCCTACAGCTCCTGGGCAACGTGCTGGTTATTGTGACCGGTCGCTAGCC
ACCATGGTGAGCAAGGGCGAGGAGCTGTTCACCGGGGTGGTGCCCATCCTGGTCGAGCTGGACG
GCGACGTAAACGGCCACAAGTTCAGCGTGTCCGGCGAGGGCGAGGGCGATGCCACCTACGGCAA
GCTGACCCTGAAGTTCATCTGCACCACCGGCAAGCTGCCCGTGCCCTGGCCCACCCTCGTGACC
ACCCTGACCTACGGCGTGCAGTGCTTCAGCCGCTACCCCGACCACATGAAGCAGCACGACTTCT
TCAAGTCCGCCATGCCCGAAGGCTACGTCCAGGAGCGCACCATCTTCTTCAAGGACGACGGCAA
CTACAAGACCCGCGCCGAGGTGAAGTTCGAGGGCGACACCCTGGTGAACCGCATCGAGCTGAAG
GGCATCGACTTCAAGGAGGACGGCAACATCCTGGGGCACAAGCTGGAGTACAACTACAACAGCC
ACAACGTCTATATCATGGCCGACAAGCAGAAGAACGGCATCAAGGTGAACTTCAAGATCCGCCA
CAACATCGAGGACGGCAGCGTGCAGCTCGCCGACCACTACCAGCAGAACACCCCCATCGGCGAC
GGCCCCGTGCTGCTGCCCGACAACCACTACCTGAGCACCCAGTCCGCCCTGAGCAAAGACCCCA
ACGAGAAGCGCGATCACATGGTCCTGCTGGAGTTCGTGACCGCCGCCGGGATCACTCTCGGCAT
GGACGAGCTGTACAAGTAAcctgcaggcgccggcgCATAGCTCTTATGATAGCAATGTCAGCAG
TGCCTTTCTCGAAGTGCTTCTGCCGGGTTAAGATTCTAAAATTATCACGGTAGAAACATCTTCG
GGAGAAGTAAGGTTGACCATACTCTACAGTTGTGGAGCTCGCTGATCAGCCTCGACTGTGCCTT
CTAGTTGCCAGCCATCTGTTGTTTGCCCCTCCCCCGTGCCTTCCTTGACCCTGGAAGGTGCCAC
TCCCACTGTCCTTTCCTAATAAAATGAGGAAATTGCATCGCATTGTCTGAGTAGGTGTCATTCT
ATTCTGGGGGGTGGGGTGGGGCAGGACAGCAAGGGGGAGGATTGGGAAGACAATAGCAGGCATG
CTGGGGATGCGGTGGGCTCTATGGAAGCTTGAATTCAGCTGACGTGCCTCGGACCGCTAGGAAC
CCCTAGTGATGGAGTTGGCCACTCCCTCTCTGCGCGCTCGCTCGCTCACTGAGGCCGGGCGACC
AAAGGTCGCCCGACGCCCGGGCTTTGCCCGGGCGGCCTCAGTGAGCGAGCGAGCGCGCAG
Exemplary pITR.CAG.miR4-26_EGFP Construct sequence
(SEQ ID NO: 185)
CTGCGCGCTCGCTCGCTCACTGAGGCCGCCCGGGCGTCGGGCGACCTTTGGTCGCCCGGCCTCA
GTGAGCGAGCGAGCGCGCAGAGAGGGAGTGGCCAACTCCATCACTAGGGGTTCCTGCGGCCGCA
CGCGTGACATTGATTATTGACTAGTTATTAATAGTAATCAATTACGGGGTCATTAGTTCATAGC
CCATATATGGAGTTCCGCGTTACATAACTTACGGTAAATGGCCCGCCTGGCTGACCGCCCAACG
ACCCCCGCCCATTGACGTCAATAATGACGTATGTTCCCATAGTAACGCCAATAGGGACTTTCCA
TTGACGTCAATGGGTGGACTATTTACGGTAAACTGCCCACTTGGCAGTACATCAAGTGTATCAT
ATGCCAAGTACGCCCCCTATTGACGTCAATGACGGTAAATGGCCCGCCTGGCATTATGCCCAGT
ACATGACCTTATGGGACTTTCCTACTTGGCAGTACATCTACGTATTAGTCATCGCTATTACCAT
GGGTCGAGGTGAGCCCCACGTTCTGCTTCACTCTCCCCATCTCCCCCCCCTCCCCACCCCCAAT
TTTGTATTTATTTATTTTTTAATTATTTTGTGCAGCGATGGGGGCGGGGGGGGGGGGGGCGCGC
GCCAGGCGGGGCGGGGCGGGGCGAGGGGCGGGGCGGGGCGAGGCGGAGAGGTGCGGCGGCAGCC
AATCAGAGCGGCGCGCTCCGAAAGTTTCCTTTTATGGCGAGGCGGCGGCGGCGGCGGCCCTATA
AAAAGCGAAGCGCGCGGCGGGCGGGAGTCGCTGCGTTGCCTTCGCCCCGTGCCCCGCTCCGCGC
CGCCTCGCGCCGCCCGCCCCGGCTCTGACTGACCGCGTTACTCCCACAGGTGAGCGGGCGGGAC
GGCCCTTCTCCTCCGGGCTGTAATTAGCGCTTGGTTTAATGACGGCTCGTTTCTTTTCTGTGGC
TGCGTGAAAGCCTTAAAGGGCTCCGGGAGGGCCCTTTGTGCGGGGGGGAGCGGCTCGGGGGGTG
CGTGCGTGTGTGTGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGCGGCCGGGGGCGG
TGCCCCGCGGTGCGGGGGGGCTGCGAGGGGAACAAAGGCTGCGTGCGGGGTGTGTGCGTGGGGG
GGTGAGCAGGGGGTGTGGGCGCGGCGGTCGGGCTGTAACCCCCCCCTGCACCCCCCTCCCCGAG
TTGCTGAGCACGGCCCGGCTTCGGGTGCGGGGCTCCGTGCGGGGCGTGGCGCGGGGCTCGCCGT
GCCGGGCGGGGGGTGGCGGCAGGTGGGGGTGCCGGGCGGGGCGGGGCCGCCTCGGGCCGGGGAG
GGCTCGGGGGAGGGGCGCGGCGGCCCCCGGAGCGCCGGCGGAGCAGGAGGACCAAGGCCCTGGC
GAAGGCCGTGGCCTCGTTCTCGAAGTGCTTCTGCCGGTGTGCAGGTCCCAATGGCCGGTAGAAT
CGTTTCGAGCACGGGGACGCGGGCCTGGACGCCGGCATCCGGGCTCAGGACCCCCCTCTCTAGA
GCCTCTGCTAACCATGTTCATGCCTTCTTCTTTTTCCTACAGCTCCTGGGCAACGTGCTGGTTA
TTGTGACCGGTCGCTAGCCACCATGGTGAGCAAGGGCGAGGAGCTGTTCACCGGGGTGGTGCCC
ATCCTGGTCGAGCTGGACGGCGACGTAAACGGCCACAAGTTCAGCGTGTCCGGCGAGGGCGAGG
GCGATGCCACCTACGGCAAGCTGACCCTGAAGTTCATCTGCACCACCGGCAAGCTGCCCGTGCC
CTGGCCCACCCTCGTGACCACCCTGACCTACGGCGTGCAGTGCTTCAGCCGCTACCCCGACCAC
ATGAAGCAGCACGACTTCTTCAAGTCCGCCATGCCCGAAGGCTACGTCCAGGAGCGCACCATCT
TCTTCAAGGACGACGGCAACTACAAGACCCGCGCCGAGGTGAAGTTCGAGGGCGACACCCTGGT
GAACCGCATCGAGCTGAAGGGCATCGACTTCAAGGAGGACGGCAACATCCTGGGGCACAAGCTG
GAGTACAACTACAACAGCCACAACGTCTATATCATGGCCGACAAGCAGAAGAACGGCATCAAGG
TGAACTTCAAGATCCGCCACAACATCGAGGACGGCAGCGTGCAGCTCGCCGACCACTACCAGCA
GAACACCCCCATCGGCGACGGCCCCGTGCTGCTGCCCGACAACCACTACCTGAGCACCCAGTCC
GCCCTGAGCAAAGACCCCAACGAGAAGCGCGATCACATGGTCCTGCTGGAGTTCGTGACCGCCG
CCGGGATCACTCTCGGCATGGACGAGCTGTACAAGTAAcctgcaggcgccggcgGAGCAGGAGG
ACCAAGGCCCTGGCGAAGGCCGTGGCCTCGTTCTCGAAGTGCTTCTGCCGGTGTGCAGGTCCCA
ATGGCCGGTAGAATCGTTTCGAGCACGGGGACGCGGGCCTGGACGCCGGCATCCGGGCTCAGGA
CCCCCCTCGAGCTCGCTGATCAGCCTCGACTGTGCCTTCTAGTTGCCAGCCATCTGTTGTTTGC
CCCTCCCCCGTGCCTTCCTTGACCCTGGAAGGTGCCACTCCCACTGTCCTTTCCTAATAAAATG
AGGAAATTGCATCGCATTGTCTGAGTAGGTGTCATTCTATTCTGGGGGGTGGGGTGGGGCAGGA
CAGCAAGGGGGAGGATTGGGAAGACAATAGCAGGCATGCTGGGGATGCGGTGGGCTCTATGGAA
GCTTGAATTCAGCTGACGTGCCTCGGACCGCTAGGAACCCCTAGTGATGGAGTTGGCCACTCCC
TCTCTGCGCGCTCGCTCGCTCACTGAGGCCGGGCGACCAAAGGTCGCCCGACGCCCGGGCTTTG
CCCGGGCGGCCTCAGTGAGCGAGCGAGCGCGCAG
Exemplary pITR.CAG.miR4-96_EGFP Construct sequence
(SEQ ID NO: 186)
CTGCGCGCTCGCTCGCTCACTGAGGCCGCCCGGGCGTCGGGCGACCTTTGGTCGCCCGGCCTCA
GTGAGCGAGCGAGCGCGCAGAGAGGGAGTGGCCAACTCCATCACTAGGGGTTCCTGCGGCCGCA
CGCGTGACATTGATTATTGACTAGTTATTAATAGTAATCAATTACGGGGTCATTAGTTCATAGC
CCATATATGGAGTTCCGCGTTACATAACTTACGGTAAATGGCCCGCCTGGCTGACCGCCCAACG
ACCCCCGCCCATTGACGTCAATAATGACGTATGTTCCCATAGTAACGCCAATAGGGACTTTCCA
TTGACGTCAATGGGTGGACTATTTACGGTAAACTGCCCACTTGGCAGTACATCAAGTGTATCAT
ATGCCAAGTACGCCCCCTATTGACGTCAATGACGGTAAATGGCCCGCCTGGCATTATGCCCAGT
ACATGACCTTATGGGACTTTCCTACTTGGCAGTACATCTACGTATTAGTCATCGCTATTACCAT
GGGTCGAGGTGAGCCCCACGTTCTGCTTCACTCTCCCCATCTCCCCCCCCTCCCCACCCCCAAT
TTTGTATTTATTTATTTTTTAATTATTTTGTGCAGCGATGGGGGCGGGGGGGGGGGGGGCGCGC
GCCAGGCGGGGCGGGGCGGGGCGAGGGGCGGGGCGGGGCGAGGCGGAGAGGTGCGGCGGCAGCC
AATCAGAGCGGCGCGCTCCGAAAGTTTCCTTTTATGGCGAGGCGGCGGCGGCGGCGGCCCTATA
AAAAGCGAAGCGCGCGGCGGGCGGGAGTCGCTGCGTTGCCTTCGCCCCGTGCCCCGCTCCGCGC
CGCCTCGCGCCGCCCGCCCCGGCTCTGACTGACCGCGTTACTCCCACAGGTGAGCGGGCGGGAC
GGCCCTTCTCCTCCGGGCTGTAATTAGCGCTTGGTTTAATGACGGCTCGTTTCTTTTCTGTGGC
TGCGTGAAAGCCTTAAAGGGCTCCGGGAGGGCCCTTTGTGCGGGGGGGAGCGGCTCGGGGGGTG
CGTGCGTGTGTGTGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGCGGCCGGGGGCGG
TGCCCCGCGGTGCGGGGGGGCTGCGAGGGGAACAAAGGCTGCGTGCGGGGTGTGTGCGTGGGGG
GGTGAGCAGGGGGTGTGGGCGCGGCGGTCGGGCTGTAACCCCCCCCTGCACCCCCCTCCCCGAG
TTGCTGAGCACGGCCCGGCTTCGGGTGCGGGGCTCCGTGCGGGGCGTGGCGCGGGGCTCGCCGT
GCCGGGCGGGGGGTGGCGGCAGGTGGGGGTGCCGGGCGGGGCGGGGCCGCCTCGGGCCGGGGAG
GGCTCGGGGGAGGGGCGCGGCGGCCCCCGGAGCGCCGGCGGTGTCCCCAGAGAGCCCGCACCAG
TGCCATCTGCTTGGCCGATTTCTCGAAGTGCTTCTGCCGGCTTGTGTCTCTCCGCTCTGAGCTG
TGAGGAGCTTTCGGGATATGGGAAAAGCAGGACCCGCAGCTGCGTCCGCCTCCCCTGCATTCTA
GAGCCTCTGCTAACCATGTTCATGCCTTCTTCTTTTTCCTACAGCTCCTGGGCAACGTGCTGGT
TATTGTGACCGGTCGCTAGCCACCATGGTGAGCAAGGGCGAGGAGCTGTTCACCGGGGTGGTGC
CCATCCTGGTCGAGCTGGACGGCGACGTAAACGGCCACAAGTTCAGCGTGTCCGGCGAGGGCGA
GGGCGATGCCACCTACGGCAAGCTGACCCTGAAGTTCATCTGCACCACCGGCAAGCTGCCCGTG
CCCTGGCCCACCCTCGTGACCACCCTGACCTACGGCGTGCAGTGCTTCAGCCGCTACCCCGACC
ACATGAAGCAGCACGACTTCTTCAAGTCCGCCATGCCCGAAGGCTACGTCCAGGAGCGCACCAT
CTTCTTCAAGGACGACGGCAACTACAAGACCCGCGCCGAGGTGAAGTTCGAGGGCGACACCCTG
GTGAACCGCATCGAGCTGAAGGGCATCGACTTCAAGGAGGACGGCAACATCCTGGGGCACAAGC
TGGAGTACAACTACAACAGCCACAACGTCTATATCATGGCCGACAAGCAGAAGAACGGCATCAA
GGTGAACTTCAAGATCCGCCACAACATCGAGGACGGCAGCGTGCAGCTCGCCGACCACTACCAG
CAGAACACCCCCATCGGCGACGGCCCCGTGCTGCTGCCCGACAACCACTACCTGAGCACCCAGT
CCGCCCTGAGCAAAGACCCCAACGAGAAGCGCGATCACATGGTCCTGCTGGAGTTCGTGACCGC
CGCCGGGATCACTCTCGGCATGGACGAGCTGTACAAGTAAcctgcaggcgccggcgGTGTCCCC
AGAGAGCCCGCACCAGTGCCATCTGCTTGGCCGATTTCTCGAAGTGCTTCTGCCGGCTTGTGTC
TCTCCGCTCTGAGCTGTGAGGAGCTTTCGGGATATGGGAAAAGCAGGACCCGCAGCTGCGTCCG
CCTCCCCTGCATGAGCTCGCTGATCAGCCTCGACTGTGCCTTCTAGTTGCCAGCCATCTGTTGT
TTGCCCCTCCCCCGTGCCTTCCTTGACCCTGGAAGGTGCCACTCCCACTGTCCTTTCCTAATAA
AATGAGGAAATTGCATCGCATTGTCTGAGTAGGTGTCATTCTATTCTGGGGGGTGGGGTGGGGC
AGGACAGCAAGGGGGAGGATTGGGAAGACAATAGCAGGCATGCTGGGGATGCGGTGGGCTCTAT
GGAAGCTTGAATTCAGCTGACGTGCCTCGGACCGCTAGGAACCCCTAGTGATGGAGTTGGCCAC
TCCCTCTCTGCGCGCTCGCTCGCTCACTGAGGCCGGGCGACCAAAGGTCGCCCGACGCCCGGGC
TTTGCCCGGGCGGCCTCAGTGAGCGAGCGAGCGCGCAG
Exemplary pITR.CAG.miR4i_miR5u-16_EGFP Construct sequence
(SEQ ID NO: 187)
CTGCGCGCTCGCTCGCTCACTGAGGCCGCCCGGGCGTCGGGCGACCTTTGGTCGCCCGGCCTCA
GTGAGCGAGCGAGCGCGCAGAGAGGGAGTGGCCAACTCCATCACTAGGGGTTCCTGCGGCCGCA
CGCGTGACATTGATTATTGACTAGTTATTAATAGTAATCAATTACGGGGTCATTAGTTCATAGC
CCATATATGGAGTTCCGCGTTACATAACTTACGGTAAATGGCCCGCCTGGCTGACCGCCCAACG
ACCCCCGCCCATTGACGTCAATAATGACGTATGTTCCCATAGTAACGCCAATAGGGACTTTCCA
TTGACGTCAATGGGTGGACTATTTACGGTAAACTGCCCACTTGGCAGTACATCAAGTGTATCAT
ATGCCAAGTACGCCCCCTATTGACGTCAATGACGGTAAATGGCCCGCCTGGCATTATGCCCAGT
ACATGACCTTATGGGACTTTCCTACTTGGCAGTACATCTACGTATTAGTCATCGCTATTACCAT
GGGTCGAGGTGAGCCCCACGTTCTGCTTCACTCTCCCCATCTCCCCCCCCTCCCCACCCCCAAT
TTTGTATTTATTTATTTTTTAATTATTTTGTGCAGCGATGGGGGCGGGGGGGGGGGGGGCGCGC
GCCAGGCGGGGCGGGGCGGGGCGAGGGGCGGGGCGGGGCGAGGCGGAGAGGTGCGGCGGCAGCC
AATCAGAGCGGCGCGCTCCGAAAGTTTCCTTTTATGGCGAGGCGGCGGCGGCGGCGGCCCTATA
AAAAGCGAAGCGCGCGGCGGGCGGGAGTCGCTGCGTTGCCTTCGCCCCGTGCCCCGCTCCGCGC
CGCCTCGCGCCGCCCGCCCCGGCTCTGACTGACCGCGTTACTCCCACAGGTGAGCGGGCGGGAC
GGCCCTTCTCCTCCGGGCTGTAATTAGCGCTTGGTTTAATGACGGCTCGTTTCTTTTCTGTGGC
TGCGTGAAAGCCTTAAAGGGCTCCGGGAGGGCCCTTTGTGCGGGGGGGAGCGGCTCGGGGGGTG
CGTGCGTGTGTGTGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGCGGCCGGGGGCGG
TGCCCCGCGGTGCGGGGGGGCTGCGAGGGGAACAAAGGCTGCGTGCGGGGTGTGTGCGTGGGGG
GGTGAGCAGGGGGTGTGGGCGCGGCGGTCGGGCTGTAACCCCCCCCTGCACCCCCCTCCCCGAG
TTGCTGAGCACGGCCCGGCTTCGGGTGCGGGGCTCCGTGCGGGGCGTGGCGCGGGGCTCGCCGT
GCCGGGCGGGGGGTGGCGGCAGGTGGGGGTGCCGGGCGGGGCGGGGCCGCCTCGGGCCGGGGAG
GGCTCGGGGGAGGGGCGCGGCGGCCCCCGGAGCGCCGGCGCATAGCTCTTATGATAGCAATGTC
AGCAGTGCCTTTCTCGAAGTGCTTCTGCCGGGTTAAGATTCTAAAATTATCACGGTAGAAACAT
CTTCGGGAGAAGTAAGGTTGACCATACTCTACAGTTGTGTCTAGAGCCTCTGCTAACCATGTTC
ATGCCTTCTTCTTTTTCCTACAGCTCCTGGGCAACGTGCTGGTTATTGTGACCGGTCGCTAGCC
ACCATGGTGAGCAAGGGCGAGGAGCTGTTCACCGGGGTGGTGCCCATCCTGGTCGAGCTGGACG
GCGACGTAAACGGCCACAAGTTCAGCGTGTCCGGCGAGGGCGAGGGCGATGCCACCTACGGCAA
GCTGACCCTGAAGTTCATCTGCACCACCGGCAAGCTGCCCGTGCCCTGGCCCACCCTCGTGACC
ACCCTGACCTACGGCGTGCAGTGCTTCAGCCGCTACCCCGACCACATGAAGCAGCACGACTTCT
TCAAGTCCGCCATGCCCGAAGGCTACGTCCAGGAGCGCACCATCTTCTTCAAGGACGACGGCAA
CTACAAGACCCGCGCCGAGGTGAAGTTCGAGGGCGACACCCTGGTGAACCGCATCGAGCTGAAG
GGCATCGACTTCAAGGAGGACGGCAACATCCTGGGGCACAAGCTGGAGTACAACTACAACAGCC
ACAACGTCTATATCATGGCCGACAAGCAGAAGAACGGCATCAAGGTGAACTTCAAGATCCGCCA
CAACATCGAGGACGGCAGCGTGCAGCTCGCCGACCACTACCAGCAGAACACCCCCATCGGCGAC
GGCCCCGTGCTGCTGCCCGACAACCACTACCTGAGCACCCAGTCCGCCCTGAGCAAAGACCCCA
ACGAGAAGCGCGATCACATGGTCCTGCTGGAGTTCGTGACCGCCGCCGGGATCACTCTCGGCAT
GGACGAGCTGTACAAGTAAcctgcaggcgccggcgCATAGCTCTTATGATAGCAATGTCAGCAG
TGCCTTTCTCCACCTTGACCACGCGTGTTAAGATTCTAAAATTATCTCGTGTGGTAAGTGGTGG
AGAGAAGTAAGGTTGACCATACTCTACAGTTGTGGAGCTCGCTGATCAGCCTCGACTGTGCCTT
CTAGTTGCCAGCCATCTGTTGTTTGCCCCTCCCCCGTGCCTTCCTTGACCCTGGAAGGTGCCAC
TCCCACTGTCCTTTCCTAATAAAATGAGGAAATTGCATCGCATTGTCTGAGTAGGTGTCATTCT
ATTCTGGGGGGTGGGGTGGGGCAGGACAGCAAGGGGGAGGATTGGGAAGACAATAGCAGGCATG
CTGGGGATGCGGTGGGCTCTATGGAAGCTTGAATTCAGCTGACGTGCCTCGGACCGCTAGGAAC
CCCTAGTGATGGAGTTGGCCACTCCCTCTCTGCGCGCTCGCTCGCTCACTGAGGCCGGGCGACC
AAAGGTCGCCCGACGCCCGGGCTTTGCCCGGGCGGCCTCAGTGAGCGAGCGAGCGCGCAG
Exemplary pITR.CAG.miR4i_miR5u-26_EGFP Construct sequence
(SEQ ID NO: 188)
CTGCGCGCTCGCTCGCTCACTGAGGCCGCCCGGGCGTCGGGCGACCTTTGGTCGCCCGGCCTCA
GTGAGCGAGCGAGCGCGCAGAGAGGGAGTGGCCAACTCCATCACTAGGGGTTCCTGCGGCCGCA
CGCGTGACATTGATTATTGACTAGTTATTAATAGTAATCAATTACGGGGTCATTAGTTCATAGC
CCATATATGGAGTTCCGCGTTACATAACTTACGGTAAATGGCCCGCCTGGCTGACCGCCCAACG
ACCCCCGCCCATTGACGTCAATAATGACGTATGTTCCCATAGTAACGCCAATAGGGACTTTCCA
TTGACGTCAATGGGTGGACTATTTACGGTAAACTGCCCACTTGGCAGTACATCAAGTGTATCAT
ATGCCAAGTACGCCCCCTATTGACGTCAATGACGGTAAATGGCCCGCCTGGCATTATGCCCAGT
ACATGACCTTATGGGACTTTCCTACTTGGCAGTACATCTACGTATTAGTCATCGCTATTACCAT
GGGTCGAGGTGAGCCCCACGTTCTGCTTCACTCTCCCCATCTCCCCCCCCTCCCCACCCCCAAT
TTTGTATTTATTTATTTTTTAATTATTTTGTGCAGCGATGGGGGCGGGGGGGGGGGGGGCGCGC
GCCAGGCGGGGCGGGGCGGGGCGAGGGGCGGGGCGGGGCGAGGCGGAGAGGTGCGGCGGCAGCC
AATCAGAGCGGCGCGCTCCGAAAGTTTCCTTTTATGGCGAGGCGGCGGCGGCGGCGGCCCTATA
AAAAGCGAAGCGCGCGGCGGGCGGGAGTCGCTGCGTTGCCTTCGCCCCGTGCCCCGCTCCGCGC
CGCCTCGCGCCGCCCGCCCCGGCTCTGACTGACCGCGTTACTCCCACAGGTGAGCGGGCGGGAC
GGCCCTTCTCCTCCGGGCTGTAATTAGCGCTTGGTTTAATGACGGCTCGTTTCTTTTCTGTGGC
TGCGTGAAAGCCTTAAAGGGCTCCGGGAGGGCCCTTTGTGCGGGGGGGAGCGGCTCGGGGGGTG
CGTGCGTGTGTGTGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGCGGCCGGGGGCGG
TGCCCCGCGGTGCGGGGGGGCTGCGAGGGGAACAAAGGCTGCGTGCGGGGTGTGTGCGTGGGGG
GGTGAGCAGGGGGTGTGGGCGCGGCGGTCGGGCTGTAACCCCCCCCTGCACCCCCCTCCCCGAG
TTGCTGAGCACGGCCCGGCTTCGGGTGCGGGGCTCCGTGCGGGGCGTGGCGCGGGGCTCGCCGT
GCCGGGCGGGGGGTGGCGGCAGGTGGGGGTGCCGGGCGGGGCGGGGCCGCCTCGGGCCGGGGAG
GGCTCGGGGGAGGGGCGCGGCGGCCCCCGGAGCGCCGGCGGAGCAGGAGGACCAAGGCCCTGGC
GAAGGCCGTGGCCTCGTTCTCGAAGTGCTTCTGCCGGTGTGCAGGTCCCAATGGCCGGTAGAAT
CGTTTCGAGCACGGGGACGCGGGCCTGGACGCCGGCATCCGGGCTCAGGACCCCCCTCTCTAGA
GCCTCTGCTAACCATGTTCATGCCTTCTTCTTTTTCCTACAGCTCCTGGGCAACGTGCTGGTTA
TTGTGACCGGTCGCTAGCCACCATGGTGAGCAAGGGCGAGGAGCTGTTCACCGGGGTGGTGCCC
ATCCTGGTCGAGCTGGACGGCGACGTAAACGGCCACAAGTTCAGCGTGTCCGGCGAGGGCGAGG
GCGATGCCACCTACGGCAAGCTGACCCTGAAGTTCATCTGCACCACCGGCAAGCTGCCCGTGCC
CTGGCCCACCCTCGTGACCACCCTGACCTACGGCGTGCAGTGCTTCAGCCGCTACCCCGACCAC
ATGAAGCAGCACGACTTCTTCAAGTCCGCCATGCCCGAAGGCTACGTCCAGGAGCGCACCATCT
TCTTCAAGGACGACGGCAACTACAAGACCCGCGCCGAGGTGAAGTTCGAGGGCGACACCCTGGT
GAACCGCATCGAGCTGAAGGGCATCGACTTCAAGGAGGACGGCAACATCCTGGGGCACAAGCTG
GAGTACAACTACAACAGCCACAACGTCTATATCATGGCCGACAAGCAGAAGAACGGCATCAAGG
TGAACTTCAAGATCCGCCACAACATCGAGGACGGCAGCGTGCAGCTCGCCGACCACTACCAGCA
GAACACCCCCATCGGCGACGGCCCCGTGCTGCTGCCCGACAACCACTACCTGAGCACCCAGTCC
GCCCTGAGCAAAGACCCCAACGAGAAGCGCGATCACATGGTCCTGCTGGAGTTCGTGACCGCCG
CCGGGATCACTCTCGGCATGGACGAGCTGTACAAGTAAcctgcaggcgccggcgGAGCAGGAGG
ACCAAGGCCCTGGCGAAGGCCGTGGCCTCGTTCTCCACCTTGACCACGCGTTGTGCAGGTCCCA
ATGGGCGTGTGGTAGAGGTGGAGCACGGGGACGCGGGCCTGGACGCCGGCATCCGGGCTCAGGA
CCCCCCTCGAGCTCGCTGATCAGCCTCGACTGTGCCTTCTAGTTGCCAGCCATCTGTTGTTTGC
CCCTCCCCCGTGCCTTCCTTGACCCTGGAAGGTGCCACTCCCACTGTCCTTTCCTAATAAAATG
AGGAAATTGCATCGCATTGTCTGAGTAGGTGTCATTCTATTCTGGGGGGTGGGGTGGGGCAGGA
CAGCAAGGGGGAGGATTGGGAAGACAATAGCAGGCATGCTGGGGATGCGGTGGGCTCTATGGAA
GCTTGAATTCAGCTGACGTGCCTCGGACCGCTAGGAACCCCTAGTGATGGAGTTGGCCACTCCC
TCTCTGCGCGCTCGCTCGCTCACTGAGGCCGGGCGACCAAAGGTCGCCCGACGCCCGGGCTTTG
CCCGGGCGGCCTCAGTGAGCGAGCGAGCGCGCAG
Exemplary pITR.CAG.miR4i_miR5u-96_EGFP Construct sequence
(SEQ ID NO: 189)
CTGCGCGCTCGCTCGCTCACTGAGGCCGCCCGGGCGTCGGGCGACCTTTGGTCGCCCGGCCTCA
GTGAGCGAGCGAGCGCGCAGAGAGGGAGTGGCCAACTCCATCACTAGGGGTTCCTGCGGCCGCA
CGCGTGACATTGATTATTGACTAGTTATTAATAGTAATCAATTACGGGGTCATTAGTTCATAGC
CCATATATGGAGTTCCGCGTTACATAACTTACGGTAAATGGCCCGCCTGGCTGACCGCCCAACG
ACCCCCGCCCATTGACGTCAATAATGACGTATGTTCCCATAGTAACGCCAATAGGGACTTTCCA
TTGACGTCAATGGGTGGACTATTTACGGTAAACTGCCCACTTGGCAGTACATCAAGTGTATCAT
ATGCCAAGTACGCCCCCTATTGACGTCAATGACGGTAAATGGCCCGCCTGGCATTATGCCCAGT
ACATGACCTTATGGGACTTTCCTACTTGGCAGTACATCTACGTATTAGTCATCGCTATTACCAT
GGGTCGAGGTGAGCCCCACGTTCTGCTTCACTCTCCCCATCTCCCCCCCCTCCCCACCCCCAAT
TTTGTATTTATTTATTTTTTAATTATTTTGTGCAGCGATGGGGGCGGGGGGGGGGGGGGCGCGC
GCCAGGCGGGGCGGGGCGGGGCGAGGGGCGGGGCGGGGCGAGGCGGAGAGGTGCGGCGGCAGCC
AATCAGAGCGGCGCGCTCCGAAAGTTTCCTTTTATGGCGAGGCGGCGGCGGCGGCGGCCCTATA
AAAAGCGAAGCGCGCGGCGGGCGGGAGTCGCTGCGTTGCCTTCGCCCCGTGCCCCGCTCCGCGC
CGCCTCGCGCCGCCCGCCCCGGCTCTGACTGACCGCGTTACTCCCACAGGTGAGCGGGCGGGAC
GGCCCTTCTCCTCCGGGCTGTAATTAGCGCTTGGTTTAATGACGGCTCGTTTCTTTTCTGTGGC
TGCGTGAAAGCCTTAAAGGGCTCCGGGAGGGCCCTTTGTGCGGGGGGGAGCGGCTCGGGGGGTG
CGTGCGTGTGTGTGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGCGGCCGGGGGCGG
TGCCCCGCGGTGCGGGGGGGCTGCGAGGGGAACAAAGGCTGCGTGCGGGGTGTGTGCGTGGGGG
GGTGAGCAGGGGGTGTGGGCGCGGCGGTCGGGCTGTAACCCCCCCCTGCACCCCCCTCCCCGAG
TTGCTGAGCACGGCCCGGCTTCGGGTGCGGGGCTCCGTGCGGGGCGTGGCGCGGGGCTCGCCGT
GCCGGGCGGGGGGTGGCGGCAGGTGGGGGTGCCGGGCGGGGCGGGGCCGCCTCGGGCCGGGGAG
GGCTCGGGGGAGGGGCGCGGCGGCCCCCGGAGCGCCGGCGGTGTCCCCAGAGAGCCCGCACCAG
TGCCATCTGCTTGGCCGATTTCTCGAAGTGCTTCTGCCGGCTTGTGTCTCTCCGCTCTGAGCTG
TGAGGAGCTTTCGGGATATGGGAAAAGCAGGACCCGCAGCTGCGTCCGCCTCCCCTGCATTCTA
GAGCCTCTGCTAACCATGTTCATGCCTTCTTCTTTTTCCTACAGCTCCTGGGCAACGTGCTGGT
TATTGTGACCGGTCGCTAGCCACCATGGTGAGCAAGGGCGAGGAGCTGTTCACCGGGGTGGTGC
CCATCCTGGTCGAGCTGGACGGCGACGTAAACGGCCACAAGTTCAGCGTGTCCGGCGAGGGCGA
GGGCGATGCCACCTACGGCAAGCTGACCCTGAAGTTCATCTGCACCACCGGCAAGCTGCCCGTG
CCCTGGCCCACCCTCGTGACCACCCTGACCTACGGCGTGCAGTGCTTCAGCCGCTACCCCGACC
ACATGAAGCAGCACGACTTCTTCAAGTCCGCCATGCCCGAAGGCTACGTCCAGGAGCGCACCAT
CTTCTTCAAGGACGACGGCAACTACAAGACCCGCGCCGAGGTGAAGTTCGAGGGCGACACCCTG
GTGAACCGCATCGAGCTGAAGGGCATCGACTTCAAGGAGGACGGCAACATCCTGGGGCACAAGC
TGGAGTACAACTACAACAGCCACAACGTCTATATCATGGCCGACAAGCAGAAGAACGGCATCAA
GGTGAACTTCAAGATCCGCCACAACATCGAGGACGGCAGCGTGCAGCTCGCCGACCACTACCAG
CAGAACACCCCCATCGGCGACGGCCCCGTGCTGCTGCCCGACAACCACTACCTGAGCACCCAGT
CCGCCCTGAGCAAAGACCCCAACGAGAAGCGCGATCACATGGTCCTGCTGGAGTTCGTGACCGC
CGCCGGGATCACTCTCGGCATGGACGAGCTGTACAAGTAAcctgcaggcgccggcgGTGTCCCC
AGAGAGCCCGCACCAGTGCCATCTGCTTGGCCGATTTCTCCACCTTGACCACGCGTCTTGTGTC
TCTCCGCTCTGAGGTGACTGGTTGGGTGGAGATATGGGAAAAGCAGGACCCGCAGCTGCGTCCG
CCTCCCCTGCATGAGCTCGCTGATCAGCCTCGACTGTGCCTTCTAGTTGCCAGCCATCTGTTGT
TTGCCCCTCCCCCGTGCCTTCCTTGACCCTGGAAGGTGCCACTCCCACTGTCCTTTCCTAATAA
AATGAGGAAATTGCATCGCATTGTCTGAGTAGGTGTCATTCTATTCTGGGGGGTGGGGTGGGGC
AGGACAGCAAGGGGGAGGATTGGGAAGACAATAGCAGGCATGCTGGGGATGCGGTGGGCTCTAT
GGAAGCTTGAATTCAGCTGACGTGCCTCGGACCGCTAGGAACCCCTAGTGATGGAGTTGGCCAC
TCCCTCTCTGCGCGCTCGCTCGCTCACTGAGGCCGGGCGACCAAAGGTCGCCCGACGCCCGGGC
TTTGCCCGGGCGGCCTCAGTGAGCGAGCGAGCGCGCAG
Exemplary pITR.CAG.miR4i_miR6u-16_EGFP Construct sequence
(SEQ ID NO: 190)
CTGCGCGCTCGCTCGCTCACTGAGGCCGCCCGGGCGTCGGGCGACCTTTGGTCGCCCGGCCTCA
GTGAGCGAGCGAGCGCGCAGAGAGGGAGTGGCCAACTCCATCACTAGGGGTTCCTGCGGCCGCA
CGCGTGACATTGATTATTGACTAGTTATTAATAGTAATCAATTACGGGGTCATTAGTTCATAGC
CCATATATGGAGTTCCGCGTTACATAACTTACGGTAAATGGCCCGCCTGGCTGACCGCCCAACG
ACCCCCGCCCATTGACGTCAATAATGACGTATGTTCCCATAGTAACGCCAATAGGGACTTTCCA
TTGACGTCAATGGGTGGACTATTTACGGTAAACTGCCCACTTGGCAGTACATCAAGTGTATCAT
ATGCCAAGTACGCCCCCTATTGACGTCAATGACGGTAAATGGCCCGCCTGGCATTATGCCCAGT
ACATGACCTTATGGGACTTTCCTACTTGGCAGTACATCTACGTATTAGTCATCGCTATTACCAT
GGGTCGAGGTGAGCCCCACGTTCTGCTTCACTCTCCCCATCTCCCCCCCCTCCCCACCCCCAAT
TTTGTATTTATTTATTTTTTAATTATTTTGTGCAGCGATGGGGGCGGGGGGGGGGGGGGCGCGC
GCCAGGCGGGGCGGGGCGGGGCGAGGGGCGGGGCGGGGCGAGGCGGAGAGGTGCGGCGGCAGCC
AATCAGAGCGGCGCGCTCCGAAAGTTTCCTTTTATGGCGAGGCGGCGGCGGCGGCGGCCCTATA
AAAAGCGAAGCGCGCGGCGGGCGGGAGTCGCTGCGTTGCCTTCGCCCCGTGCCCCGCTCCGCGC
CGCCTCGCGCCGCCCGCCCCGGCTCTGACTGACCGCGTTACTCCCACAGGTGAGCGGGCGGGAC
GGCCCTTCTCCTCCGGGCTGTAATTAGCGCTTGGTTTAATGACGGCTCGTTTCTTTTCTGTGGC
TGCGTGAAAGCCTTAAAGGGCTCCGGGAGGGCCCTTTGTGCGGGGGGGAGCGGCTCGGGGGGTG
CGTGCGTGTGTGTGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGCGGCCGGGGGCGG
TGCCCCGCGGTGCGGGGGGGCTGCGAGGGGAACAAAGGCTGCGTGCGGGGTGTGTGCGTGGGGG
GGTGAGCAGGGGGTGTGGGCGCGGCGGTCGGGCTGTAACCCCCCCCTGCACCCCCCTCCCCGAG
TTGCTGAGCACGGCCCGGCTTCGGGTGCGGGGCTCCGTGCGGGGCGTGGCGCGGGGCTCGCCGT
GCCGGGCGGGGGGTGGCGGCAGGTGGGGGTGCCGGGCGGGGCGGGGCCGCCTCGGGCCGGGGAG
GGCTCGGGGGAGGGGCGCGGCGGCCCCCGGAGCGCCGGCGCATAGCTCTTATGATAGCAATGTC
AGCAGTGCCTTTCTCGAAGTGCTTCTGCCGGGTTAAGATTCTAAAATTATCACGGTAGAAACAT
CTTCGGGAGAAGTAAGGTTGACCATACTCTACAGTTGTGTCTAGAGCCTCTGCTAACCATGTTC
ATGCCTTCTTCTTTTTCCTACAGCTCCTGGGCAACGTGCTGGTTATTGTGACCGGTCGCTAGCC
ACCATGGTGAGCAAGGGCGAGGAGCTGTTCACCGGGGTGGTGCCCATCCTGGTCGAGCTGGACG
GCGACGTAAACGGCCACAAGTTCAGCGTGTCCGGCGAGGGCGAGGGCGATGCCACCTACGGCAA
GCTGACCCTGAAGTTCATCTGCACCACCGGCAAGCTGCCCGTGCCCTGGCCCACCCTCGTGACC
ACCCTGACCTACGGCGTGCAGTGCTTCAGCCGCTACCCCGACCACATGAAGCAGCACGACTTCT
TCAAGTCCGCCATGCCCGAAGGCTACGTCCAGGAGCGCACCATCTTCTTCAAGGACGACGGCAA
CTACAAGACCCGCGCCGAGGTGAAGTTCGAGGGCGACACCCTGGTGAACCGCATCGAGCTGAAG
GGCATCGACTTCAAGGAGGACGGCAACATCCTGGGGCACAAGCTGGAGTACAACTACAACAGCC
ACAACGTCTATATCATGGCCGACAAGCAGAAGAACGGCATCAAGGTGAACTTCAAGATCCGCCA
CAACATCGAGGACGGCAGCGTGCAGCTCGCCGACCACTACCAGCAGAACACCCCCATCGGCGAC
GGCCCCGTGCTGCTGCCCGACAACCACTACCTGAGCACCCAGTCCGCCCTGAGCAAAGACCCCA
ACGAGAAGCGCGATCACATGGTCCTGCTGGAGTTCGTGACCGCCGCCGGGATCACTCTCGGCAT
GGACGAGCTGTACAAGTAAcctgcaggcgccggcgCATAGCTCTTATGATAGCAATGTCAGCAG
TGCCTATACTGTCATAGTAGTACCAGGTTAAGATTCTAAAATTATCATGGTACTAATGTTGGCA
GTGTAAGTAAGGTTGACCATACTCTACAGTTGTGGAGCTCGCTGATCAGCCTCGACTGTGCCTT
CTAGTTGCCAGCCATCTGTTGTTTGCCCCTCCCCCGTGCCTTCCTTGACCCTGGAAGGTGCCAC
TCCCACTGTCCTTTCCTAATAAAATGAGGAAATTGCATCGCATTGTCTGAGTAGGTGTCATTCT
ATTCTGGGGGGTGGGGTGGGGCAGGACAGCAAGGGGGAGGATTGGGAAGACAATAGCAGGCATG
CTGGGGATGCGGTGGGCTCTATGGAAGCTTGAATTCAGCTGACGTGCCTCGGACCGCTAGGAAC
CCCTAGTGATGGAGTTGGCCACTCCCTCTCTGCGCGCTCGCTCGCTCACTGAGGCCGGGCGACC
AAAGGTCGCCCGACGCCCGGGCTTTGCCCGGGCGGCCTCAGTGAGCGAGCGAGCGCGCAG
Exemplary pITR.CAG.miR4i_miR6u-26_EGFP Construct sequence
(SEQ ID NO: 191)
CTGCGCGCTCGCTCGCTCACTGAGGCCGCCCGGGCGTCGGGCGACCTTTGGTCGCCCGGCCTCA
GTGAGCGAGCGAGCGCGCAGAGAGGGAGTGGCCAACTCCATCACTAGGGGTTCCTGCGGCCGCA
CGCGTGACATTGATTATTGACTAGTTATTAATAGTAATCAATTACGGGGTCATTAGTTCATAGC
CCATATATGGAGTTCCGCGTTACATAACTTACGGTAAATGGCCCGCCTGGCTGACCGCCCAACG
ACCCCCGCCCATTGACGTCAATAATGACGTATGTTCCCATAGTAACGCCAATAGGGACTTTCCA
TTGACGTCAATGGGTGGACTATTTACGGTAAACTGCCCACTTGGCAGTACATCAAGTGTATCAT
ATGCCAAGTACGCCCCCTATTGACGTCAATGACGGTAAATGGCCCGCCTGGCATTATGCCCAGT
ACATGACCTTATGGGACTTTCCTACTTGGCAGTACATCTACGTATTAGTCATCGCTATTACCAT
GGGTCGAGGTGAGCCCCACGTTCTGCTTCACTCTCCCCATCTCCCCCCCCTCCCCACCCCCAAT
TTTGTATTTATTTATTTTTTAATTATTTTGTGCAGCGATGGGGGCGGGGGGGGGGGGGGCGCGC
GCCAGGCGGGGCGGGGCGGGGCGAGGGGCGGGGCGGGGCGAGGCGGAGAGGTGCGGCGGCAGCC
AATCAGAGCGGCGCGCTCCGAAAGTTTCCTTTTATGGCGAGGCGGCGGCGGCGGCGGCCCTATA
AAAAGCGAAGCGCGCGGCGGGCGGGAGTCGCTGCGTTGCCTTCGCCCCGTGCCCCGCTCCGCGC
CGCCTCGCGCCGCCCGCCCCGGCTCTGACTGACCGCGTTACTCCCACAGGTGAGCGGGCGGGAC
GGCCCTTCTCCTCCGGGCTGTAATTAGCGCTTGGTTTAATGACGGCTCGTTTCTTTTCTGTGGC
TGCGTGAAAGCCTTAAAGGGCTCCGGGAGGGCCCTTTGTGCGGGGGGGAGCGGCTCGGGGGGTG
CGTGCGTGTGTGTGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGCGGCCGGGGGCGG
TGCCCCGCGGTGCGGGGGGGCTGCGAGGGGAACAAAGGCTGCGTGCGGGGTGTGTGCGTGGGGG
GGTGAGCAGGGGGTGTGGGCGCGGCGGTCGGGCTGTAACCCCCCCCTGCACCCCCCTCCCCGAG
TTGCTGAGCACGGCCCGGCTTCGGGTGCGGGGCTCCGTGCGGGGCGTGGCGCGGGGCTCGCCGT
GCCGGGCGGGGGGTGGCGGCAGGTGGGGGTGCCGGGCGGGGCGGGGCCGCCTCGGGCCGGGGAG
GGCTCGGGGGAGGGGCGCGGCGGCCCCCGGAGCGCCGGCGGAGCAGGAGGACCAAGGCCCTGGC
GAAGGCCGTGGCCTCGTTCTCGAAGTGCTTCTGCCGGTGTGCAGGTCCCAATGGCCGGTAGAAT
CGTTTCGAGCACGGGGACGCGGGCCTGGACGCCGGCATCCGGGCTCAGGACCCCCCTCTCTAGA
GCCTCTGCTAACCATGTTCATGCCTTCTTCTTTTTCCTACAGCTCCTGGGCAACGTGCTGGTTA
TTGTGACCGGTCGCTAGCCACCATGGTGAGCAAGGGCGAGGAGCTGTTCACCGGGGTGGTGCCC
ATCCTGGTCGAGCTGGACGGCGACGTAAACGGCCACAAGTTCAGCGTGTCCGGCGAGGGCGAGG
GCGATGCCACCTACGGCAAGCTGACCCTGAAGTTCATCTGCACCACCGGCAAGCTGCCCGTGCC
CTGGCCCACCCTCGTGACCACCCTGACCTACGGCGTGCAGTGCTTCAGCCGCTACCCCGACCAC
ATGAAGCAGCACGACTTCTTCAAGTCCGCCATGCCCGAAGGCTACGTCCAGGAGCGCACCATCT
TCTTCAAGGACGACGGCAACTACAAGACCCGCGCCGAGGTGAAGTTCGAGGGCGACACCCTGGT
GAACCGCATCGAGCTGAAGGGCATCGACTTCAAGGAGGACGGCAACATCCTGGGGCACAAGCTG
GAGTACAACTACAACAGCCACAACGTCTATATCATGGCCGACAAGCAGAAGAACGGCATCAAGG
TGAACTTCAAGATCCGCCACAACATCGAGGACGGCAGCGTGCAGCTCGCCGACCACTACCAGCA
GAACACCCCCATCGGCGACGGCCCCGTGCTGCTGCCCGACAACCACTACCTGAGCACCCAGTCC
GCCCTGAGCAAAGACCCCAACGAGAAGCGCGATCACATGGTCCTGCTGGAGTTCGTGACCGCCG
CCGGGATCACTCTCGGCATGGACGAGCTGTACAAGTAAcctgcaggcgccggcgGAGCAGGAGG
ACCAAGGCCCTGGCGAAGGCCGTGGCCTCGATACTGTCATAGTAGTACCAGTGTGCAGGTCCCA
ATGGCTGGTACTAATGTGACAGTCTCGGGGACGCGGGCCTGGACGCCGGCATCCGGGCTCAGGA
CCCCCCTCGAGCTCGCTGATCAGCCTCGACTGTGCCTTCTAGTTGCCAGCCATCTGTTGTTTGC
CCCTCCCCCGTGCCTTCCTTGACCCTGGAAGGTGCCACTCCCACTGTCCTTTCCTAATAAAATG
AGGAAATTGCATCGCATTGTCTGAGTAGGTGTCATTCTATTCTGGGGGGTGGGGTGGGGCAGGA
CAGCAAGGGGGAGGATTGGGAAGACAATAGCAGGCATGCTGGGGATGCGGTGGGCTCTATGGAA
GCTTGAATTCAGCTGACGTGCCTCGGACCGCTAGGAACCCCTAGTGATGGAGTTGGCCACTCCC
TCTCTGCGCGCTCGCTCGCTCACTGAGGCCGGGCGACCAAAGGTCGCCCGACGCCCGGGCTTTG
CCCGGGCGGCCTCAGTGAGCGAGCGAGCGCGCAG
Exemplary pITR.CAG.miR4i_miR6u-96_EGFP Construct sequence
(SEQ ID NO: 192)
CTGCGCGCTCGCTCGCTCACTGAGGCCGCCCGGGCGTCGGGCGACCTTTGGTCGCCCGGCCTCA
GTGAGCGAGCGAGCGCGCAGAGAGGGAGTGGCCAACTCCATCACTAGGGGTTCCTGCGGCCGCA
CGCGTGACATTGATTATTGACTAGTTATTAATAGTAATCAATTACGGGGTCATTAGTTCATAGC
CCATATATGGAGTTCCGCGTTACATAACTTACGGTAAATGGCCCGCCTGGCTGACCGCCCAACG
ACCCCCGCCCATTGACGTCAATAATGACGTATGTTCCCATAGTAACGCCAATAGGGACTTTCCA
TTGACGTCAATGGGTGGACTATTTACGGTAAACTGCCCACTTGGCAGTACATCAAGTGTATCAT
ATGCCAAGTACGCCCCCTATTGACGTCAATGACGGTAAATGGCCCGCCTGGCATTATGCCCAGT
ACATGACCTTATGGGACTTTCCTACTTGGCAGTACATCTACGTATTAGTCATCGCTATTACCAT
GGGTCGAGGTGAGCCCCACGTTCTGCTTCACTCTCCCCATCTCCCCCCCCTCCCCACCCCCAAT
TTTGTATTTATTTATTTTTTAATTATTTTGTGCAGCGATGGGGGCGGGGGGGGGGGGGGCGCGC
GCCAGGCGGGGCGGGGCGGGGCGAGGGGCGGGGCGGGGCGAGGCGGAGAGGTGCGGCGGCAGCC
AATCAGAGCGGCGCGCTCCGAAAGTTTCCTTTTATGGCGAGGCGGCGGCGGCGGCGGCCCTATA
AAAAGCGAAGCGCGCGGCGGGCGGGAGTCGCTGCGTTGCCTTCGCCCCGTGCCCCGCTCCGCGC
CGCCTCGCGCCGCCCGCCCCGGCTCTGACTGACCGCGTTACTCCCACAGGTGAGCGGGCGGGAC
GGCCCTTCTCCTCCGGGCTGTAATTAGCGCTTGGTTTAATGACGGCTCGTTTCTTTTCTGTGGC
TGCGTGAAAGCCTTAAAGGGCTCCGGGAGGGCCCTTTGTGCGGGGGGGAGCGGCTCGGGGGGTG
CGTGCGTGTGTGTGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGCGGCCGGGGGCGG
TGCCCCGCGGTGCGGGGGGGCTGCGAGGGGAACAAAGGCTGCGTGCGGGGTGTGTGCGTGGGGG
GGTGAGCAGGGGGTGTGGGCGCGGCGGTCGGGCTGTAACCCCCCCCTGCACCCCCCTCCCCGAG
TTGCTGAGCACGGCCCGGCTTCGGGTGCGGGGCTCCGTGCGGGGCGTGGCGCGGGGCTCGCCGT
GCCGGGCGGGGGGTGGCGGCAGGTGGGGGTGCCGGGCGGGGCGGGGCCGCCTCGGGCCGGGGAG
GGCTCGGGGGAGGGGCGCGGCGGCCCCCGGAGCGCCGGCGGTGTCCCCAGAGAGCCCGCACCAG
TGCCATCTGCTTGGCCGATTTCTCGAAGTGCTTCTGCCGGCTTGTGTCTCTCCGCTCTGAGCTG
TGAGGAGCTTTCGGGATATGGGAAAAGCAGGACCCGCAGCTGCGTCCGCCTCCCCTGCATTCTA
GAGCCTCTGCTAACCATGTTCATGCCTTCTTCTTTTTCCTACAGCTCCTGGGCAACGTGCTGGT
TATTGTGACCGGTCGCTAGCCACCATGGTGAGCAAGGGCGAGGAGCTGTTCACCGGGGTGGTGC
CCATCCTGGTCGAGCTGGACGGCGACGTAAACGGCCACAAGTTCAGCGTGTCCGGCGAGGGCGA
GGGCGATGCCACCTACGGCAAGCTGACCCTGAAGTTCATCTGCACCACCGGCAAGCTGCCCGTG
CCCTGGCCCACCCTCGTGACCACCCTGACCTACGGCGTGCAGTGCTTCAGCCGCTACCCCGACC
ACATGAAGCAGCACGACTTCTTCAAGTCCGCCATGCCCGAAGGCTACGTCCAGGAGCGCACCAT
CTTCTTCAAGGACGACGGCAACTACAAGACCCGCGCCGAGGTGAAGTTCGAGGGCGACACCCTG
GTGAACCGCATCGAGCTGAAGGGCATCGACTTCAAGGAGGACGGCAACATCCTGGGGCACAAGC
TGGAGTACAACTACAACAGCCACAACGTCTATATCATGGCCGACAAGCAGAAGAACGGCATCAA
GGTGAACTTCAAGATCCGCCACAACATCGAGGACGGCAGCGTGCAGCTCGCCGACCACTACCAG
CAGAACACCCCCATCGGCGACGGCCCCGTGCTGCTGCCCGACAACCACTACCTGAGCACCCAGT
CCGCCCTGAGCAAAGACCCCAACGAGAAGCGCGATCACATGGTCCTGCTGGAGTTCGTGACCGC
CGCCGGGATCACTCTCGGCATGGACGAGCTGTACAAGTAAcctgcaggcgccggcgGTGTCCCC
AGAGAGCCCGCACCAGTGCCATCTGCTTGGCCGATATACTGTCATAGTAGTACCAGCTTGTGTC
TCTCCGCTCTGAGCTGTCACTGCTTGACGGTAAATGGGAAAAGCAGGACCCGCAGCTGCGTCCG
CCTCCCCTGCATGAGCTCGCTGATCAGCCTCGACTGTGCCTTCTAGTTGCCAGCCATCTGTTGT
TTGCCCCTCCCCCGTGCCTTCCTTGACCCTGGAAGGTGCCACTCCCACTGTCCTTTCCTAATAA
AATGAGGAAATTGCATCGCATTGTCTGAGTAGGTGTCATTCTATTCTGGGGGGTGGGGTGGGGC
AGGACAGCAAGGGGGAGGATTGGGAAGACAATAGCAGGCATGCTGGGGATGCGGTGGGCTCTAT
GGAAGCTTGAATTCAGCTGACGTGCCTCGGACCGCTAGGAACCCCTAGTGATGGAGTTGGCCAC
TCCCTCTCTGCGCGCTCGCTCGCTCACTGAGGCCGGGCGACCAAAGGTCGCCCGACGCCCGGGC
TTTGCCCGGGCGGCCTCAGTGAGCGAGCGAGCGCGCAG
Exemplary pITR.CAG.miR5-16_EGFP Construct sequence
(SEQ ID NO: 193)
CTGCGCGCTCGCTCGCTCACTGAGGCCGCCCGGGCGTCGGGCGACCTTTGGTCGCCCGGCCTCA
GTGAGCGAGCGAGCGCGCAGAGAGGGAGTGGCCAACTCCATCACTAGGGGTTCCTGCGGCCGCA
CGCGTGACATTGATTATTGACTAGTTATTAATAGTAATCAATTACGGGGTCATTAGTTCATAGC
CCATATATGGAGTTCCGCGTTACATAACTTACGGTAAATGGCCCGCCTGGCTGACCGCCCAACG
ACCCCCGCCCATTGACGTCAATAATGACGTATGTTCCCATAGTAACGCCAATAGGGACTTTCCA
TTGACGTCAATGGGTGGACTATTTACGGTAAACTGCCCACTTGGCAGTACATCAAGTGTATCAT
ATGCCAAGTACGCCCCCTATTGACGTCAATGACGGTAAATGGCCCGCCTGGCATTATGCCCAGT
ACATGACCTTATGGGACTTTCCTACTTGGCAGTACATCTACGTATTAGTCATCGCTATTACCAT
GGGTCGAGGTGAGCCCCACGTTCTGCTTCACTCTCCCCATCTCCCCCCCCTCCCCACCCCCAAT
TTTGTATTTATTTATTTTTTAATTATTTTGTGCAGCGATGGGGGCGGGGGGGGGGGGGGCGCGC
GCCAGGCGGGGCGGGGCGGGGCGAGGGGCGGGGCGGGGCGAGGCGGAGAGGTGCGGCGGCAGCC
AATCAGAGCGGCGCGCTCCGAAAGTTTCCTTTTATGGCGAGGCGGCGGCGGCGGCGGCCCTATA
AAAAGCGAAGCGCGCGGCGGGCGGGAGTCGCTGCGTTGCCTTCGCCCCGTGCCCCGCTCCGCGC
CGCCTCGCGCCGCCCGCCCCGGCTCTGACTGACCGCGTTACTCCCACAGGTGAGCGGGCGGGAC
GGCCCTTCTCCTCCGGGCTGTAATTAGCGCTTGGTTTAATGACGGCTCGTTTCTTTTCTGTGGC
TGCGTGAAAGCCTTAAAGGGCTCCGGGAGGGCCCTTTGTGCGGGGGGGAGCGGCTCGGGGGGTG
CGTGCGTGTGTGTGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGCGGCCGGGGGCGG
TGCCCCGCGGTGCGGGGGGGCTGCGAGGGGAACAAAGGCTGCGTGCGGGGTGTGTGCGTGGGGG
GGTGAGCAGGGGGTGTGGGCGCGGCGGTCGGGCTGTAACCCCCCCCTGCACCCCCCTCCCCGAG
TTGCTGAGCACGGCCCGGCTTCGGGTGCGGGGCTCCGTGCGGGGCGTGGCGCGGGGCTCGCCGT
GCCGGGCGGGGGGTGGCGGCAGGTGGGGGTGCCGGGCGGGGCGGGGCCGCCTCGGGCCGGGGAG
GGCTCGGGGGAGGGGCGCGGCGGCCCCCGGAGCGCCGGCGCATAGCTCTTATGATAGCAATGTC
AGCAGTGCCTTTCTCCACCTTGACCACGCGTGTTAAGATTCTAAAATTATCTCGTGTGGTAAGT
GGTGGAGAGAAGTAAGGTTGACCATACTCTACAGTTGTGTCTAGAGCCTCTGCTAACCATGTTC
ATGCCTTCTTCTTTTTCCTACAGCTCCTGGGCAACGTGCTGGTTATTGTGACCGGTCGCTAGCC
ACCATGGTGAGCAAGGGCGAGGAGCTGTTCACCGGGGTGGTGCCCATCCTGGTCGAGCTGGACG
GCGACGTAAACGGCCACAAGTTCAGCGTGTCCGGCGAGGGCGAGGGCGATGCCACCTACGGCAA
GCTGACCCTGAAGTTCATCTGCACCACCGGCAAGCTGCCCGTGCCCTGGCCCACCCTCGTGACC
ACCCTGACCTACGGCGTGCAGTGCTTCAGCCGCTACCCCGACCACATGAAGCAGCACGACTTCT
TCAAGTCCGCCATGCCCGAAGGCTACGTCCAGGAGCGCACCATCTTCTTCAAGGACGACGGCAA
CTACAAGACCCGCGCCGAGGTGAAGTTCGAGGGCGACACCCTGGTGAACCGCATCGAGCTGAAG
GGCATCGACTTCAAGGAGGACGGCAACATCCTGGGGCACAAGCTGGAGTACAACTACAACAGCC
ACAACGTCTATATCATGGCCGACAAGCAGAAGAACGGCATCAAGGTGAACTTCAAGATCCGCCA
CAACATCGAGGACGGCAGCGTGCAGCTCGCCGACCACTACCAGCAGAACACCCCCATCGGCGAC
GGCCCCGTGCTGCTGCCCGACAACCACTACCTGAGCACCCAGTCCGCCCTGAGCAAAGACCCCA
ACGAGAAGCGCGATCACATGGTCCTGCTGGAGTTCGTGACCGCCGCCGGGATCACTCTCGGCAT
GGACGAGCTGTACAAGTAAcctgcaggcgccggcgCATAGCTCTTATGATAGCAATGTCAGCAG
TGCCTTTCTCCACCTTGACCACGCGTGTTAAGATTCTAAAATTATCTCGTGTGGTAAGTGGTGG
AGAGAAGTAAGGTTGACCATACTCTACAGTTGTGGAGCTCGCTGATCAGCCTCGACTGTGCCTT
CTAGTTGCCAGCCATCTGTTGTTTGCCCCTCCCCCGTGCCTTCCTTGACCCTGGAAGGTGCCAC
TCCCACTGTCCTTTCCTAATAAAATGAGGAAATTGCATCGCATTGTCTGAGTAGGTGTCATTCT
ATTCTGGGGGGTGGGGTGGGGCAGGACAGCAAGGGGGAGGATTGGGAAGACAATAGCAGGCATG
CTGGGGATGCGGTGGGCTCTATGGAAGCTTGAATTCAGCTGACGTGCCTCGGACCGCTAGGAAC
CCCTAGTGATGGAGTTGGCCACTCCCTCTCTGCGCGCTCGCTCGCTCACTGAGGCCGGGCGACC
AAAGGTCGCCCGACGCCCGGGCTTTGCCCGGGCGGCCTCAGTGAGCGAGCGAGCGCGCAG
Exemplary pITR.CAG.miR5-26_EGFP Construct sequence
(SEQ ID NO: 194)
CTGCGCGCTCGCTCGCTCACTGAGGCCGCCCGGGCGTCGGGCGACCTTTGGTCGCCCGGCCTCA
GTGAGCGAGCGAGCGCGCAGAGAGGGAGTGGCCAACTCCATCACTAGGGGTTCCTGCGGCCGCA
CGCGTGACATTGATTATTGACTAGTTATTAATAGTAATCAATTACGGGGTCATTAGTTCATAGC
CCATATATGGAGTTCCGCGTTACATAACTTACGGTAAATGGCCCGCCTGGCTGACCGCCCAACG
ACCCCCGCCCATTGACGTCAATAATGACGTATGTTCCCATAGTAACGCCAATAGGGACTTTCCA
TTGACGTCAATGGGTGGACTATTTACGGTAAACTGCCCACTTGGCAGTACATCAAGTGTATCAT
ATGCCAAGTACGCCCCCTATTGACGTCAATGACGGTAAATGGCCCGCCTGGCATTATGCCCAGT
ACATGACCTTATGGGACTTTCCTACTTGGCAGTACATCTACGTATTAGTCATCGCTATTACCAT
GGGTCGAGGTGAGCCCCACGTTCTGCTTCACTCTCCCCATCTCCCCCCCCTCCCCACCCCCAAT
TTTGTATTTATTTATTTTTTAATTATTTTGTGCAGCGATGGGGGCGGGGGGGGGGGGGGCGCGC
GCCAGGCGGGGCGGGGCGGGGCGAGGGGCGGGGCGGGGCGAGGCGGAGAGGTGCGGCGGCAGCC
AATCAGAGCGGCGCGCTCCGAAAGTTTCCTTTTATGGCGAGGCGGCGGCGGCGGCGGCCCTATA
AAAAGCGAAGCGCGCGGCGGGCGGGAGTCGCTGCGTTGCCTTCGCCCCGTGCCCCGCTCCGCGC
CGCCTCGCGCCGCCCGCCCCGGCTCTGACTGACCGCGTTACTCCCACAGGTGAGCGGGCGGGAC
GGCCCTTCTCCTCCGGGCTGTAATTAGCGCTTGGTTTAATGACGGCTCGTTTCTTTTCTGTGGC
TGCGTGAAAGCCTTAAAGGGCTCCGGGAGGGCCCTTTGTGCGGGGGGGAGCGGCTCGGGGGGTG
CGTGCGTGTGTGTGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGCGGCCGGGGGCGG
TGCCCCGCGGTGCGGGGGGGCTGCGAGGGGAACAAAGGCTGCGTGCGGGGTGTGTGCGTGGGGG
GGTGAGCAGGGGGTGTGGGCGCGGCGGTCGGGCTGTAACCCCCCCCTGCACCCCCCTCCCCGAG
TTGCTGAGCACGGCCCGGCTTCGGGTGCGGGGCTCCGTGCGGGGCGTGGCGCGGGGCTCGCCGT
GCCGGGCGGGGGGTGGCGGCAGGTGGGGGTGCCGGGCGGGGCGGGGCCGCCTCGGGCCGGGGAG
GGCTCGGGGGAGGGGCGCGGCGGCCCCCGGAGCGCCGGCGGAGCAGGAGGACCAAGGCCCTGGC
GAAGGCCGTGGCCTCGTTCTCCACCTTGACCACGCGTTGTGCAGGTCCCAATGGGCGTGTGGTA
GAGGTGGAGCACGGGGACGCGGGCCTGGACGCCGGCATCCGGGCTCAGGACCCCCCTCTCTAGA
GCCTCTGCTAACCATGTTCATGCCTTCTTCTTTTTCCTACAGCTCCTGGGCAACGTGCTGGTTA
TTGTGACCGGTCGCTAGCCACCATGGTGAGCAAGGGCGAGGAGCTGTTCACCGGGGTGGTGCCC
ATCCTGGTCGAGCTGGACGGCGACGTAAACGGCCACAAGTTCAGCGTGTCCGGCGAGGGCGAGG
GCGATGCCACCTACGGCAAGCTGACCCTGAAGTTCATCTGCACCACCGGCAAGCTGCCCGTGCC
CTGGCCCACCCTCGTGACCACCCTGACCTACGGCGTGCAGTGCTTCAGCCGCTACCCCGACCAC
ATGAAGCAGCACGACTTCTTCAAGTCCGCCATGCCCGAAGGCTACGTCCAGGAGCGCACCATCT
TCTTCAAGGACGACGGCAACTACAAGACCCGCGCCGAGGTGAAGTTCGAGGGCGACACCCTGGT
GAACCGCATCGAGCTGAAGGGCATCGACTTCAAGGAGGACGGCAACATCCTGGGGCACAAGCTG
GAGTACAACTACAACAGCCACAACGTCTATATCATGGCCGACAAGCAGAAGAACGGCATCAAGG
TGAACTTCAAGATCCGCCACAACATCGAGGACGGCAGCGTGCAGCTCGCCGACCACTACCAGCA
GAACACCCCCATCGGCGACGGCCCCGTGCTGCTGCCCGACAACCACTACCTGAGCACCCAGTCC
GCCCTGAGCAAAGACCCCAACGAGAAGCGCGATCACATGGTCCTGCTGGAGTTCGTGACCGCCG
CCGGGATCACTCTCGGCATGGACGAGCTGTACAAGTAAcctgcaggcgccggcgGAGCAGGAGG
ACCAAGGCCCTGGCGAAGGCCGTGGCCTCGTTCTCCACCTTGACCACGCGTTGTGCAGGTCCCA
ATGGGCGTGTGGTAGAGGTGGAGCACGGGGACGCGGGCCTGGACGCCGGCATCCGGGCTCAGGA
CCCCCCTCGAGCTCGCTGATCAGCCTCGACTGTGCCTTCTAGTTGCCAGCCATCTGTTGTTTGC
CCCTCCCCCGTGCCTTCCTTGACCCTGGAAGGTGCCACTCCCACTGTCCTTTCCTAATAAAATG
AGGAAATTGCATCGCATTGTCTGAGTAGGTGTCATTCTATTCTGGGGGGTGGGGTGGGGCAGGA
CAGCAAGGGGGAGGATTGGGAAGACAATAGCAGGCATGCTGGGGATGCGGTGGGCTCTATGGAA
GCTTGAATTCAGCTGACGTGCCTCGGACCGCTAGGAACCCCTAGTGATGGAGTTGGCCACTCCC
TCTCTGCGCGCTCGCTCGCTCACTGAGGCCGGGCGACCAAAGGTCGCCCGACGCCCGGGCTTTG
CCCGGGCGGCCTCAGTGAGCGAGCGAGCGCGCAG
Exemplary pITR.CAG.miR5-96_EGFP Construct sequence
(SEQ ID NO: 195)
CTGCGCGCTCGCTCGCTCACTGAGGCCGCCCGGGCGTCGGGCGACCTTTGGTCGCCCGGCCTCA
GTGAGCGAGCGAGCGCGCAGAGAGGGAGTGGCCAACTCCATCACTAGGGGTTCCTGCGGCCGCA
CGCGTGACATTGATTATTGACTAGTTATTAATAGTAATCAATTACGGGGTCATTAGTTCATAGC
CCATATATGGAGTTCCGCGTTACATAACTTACGGTAAATGGCCCGCCTGGCTGACCGCCCAACG
ACCCCCGCCCATTGACGTCAATAATGACGTATGTTCCCATAGTAACGCCAATAGGGACTTTCCA
TTGACGTCAATGGGTGGACTATTTACGGTAAACTGCCCACTTGGCAGTACATCAAGTGTATCAT
ATGCCAAGTACGCCCCCTATTGACGTCAATGACGGTAAATGGCCCGCCTGGCATTATGCCCAGT
ACATGACCTTATGGGACTTTCCTACTTGGCAGTACATCTACGTATTAGTCATCGCTATTACCAT
GGGTCGAGGTGAGCCCCACGTTCTGCTTCACTCTCCCCATCTCCCCCCCCTCCCCACCCCCAAT
TTTGTATTTATTTATTTTTTAATTATTTTGTGCAGCGATGGGGGCGGGGGGGGGGGGGGCGCGC
GCCAGGCGGGGCGGGGCGGGGCGAGGGGCGGGGCGGGGCGAGGCGGAGAGGTGCGGCGGCAGCC
AATCAGAGCGGCGCGCTCCGAAAGTTTCCTTTTATGGCGAGGCGGCGGCGGCGGCGGCCCTATA
AAAAGCGAAGCGCGCGGCGGGCGGGAGTCGCTGCGTTGCCTTCGCCCCGTGCCCCGCTCCGCGC
CGCCTCGCGCCGCCCGCCCCGGCTCTGACTGACCGCGTTACTCCCACAGGTGAGCGGGCGGGAC
GGCCCTTCTCCTCCGGGCTGTAATTAGCGCTTGGTTTAATGACGGCTCGTTTCTTTTCTGTGGC
TGCGTGAAAGCCTTAAAGGGCTCCGGGAGGGCCCTTTGTGCGGGGGGGAGCGGCTCGGGGGGTG
CGTGCGTGTGTGTGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGCGGCCGGGGGCGG
TGCCCCGCGGTGCGGGGGGGCTGCGAGGGGAACAAAGGCTGCGTGCGGGGTGTGTGCGTGGGGG
GGTGAGCAGGGGGTGTGGGCGCGGCGGTCGGGCTGTAACCCCCCCCTGCACCCCCCTCCCCGAG
TTGCTGAGCACGGCCCGGCTTCGGGTGCGGGGCTCCGTGCGGGGCGTGGCGCGGGGCTCGCCGT
GCCGGGCGGGGGGTGGCGGCAGGTGGGGGTGCCGGGCGGGGCGGGGCCGCCTCGGGCCGGGGAG
GGCTCGGGGGAGGGGCGCGGCGGCCCCCGGAGCGCCGGCGGTGTCCCCAGAGAGCCCGCACCAG
TGCCATCTGCTTGGCCGATTTCTCCACCTTGACCACGCGTCTTGTGTCTCTCCGCTCTGAGGTG
ACTGGTTGGGTGGAGATATGGGAAAAGCAGGACCCGCAGCTGCGTCCGCCTCCCCTGCATTCTA
GAGCCTCTGCTAACCATGTTCATGCCTTCTTCTTTTTCCTACAGCTCCTGGGCAACGTGCTGGT
TATTGTGACCGGTCGCTAGCCACCATGGTGAGCAAGGGCGAGGAGCTGTTCACCGGGGTGGTGC
CCATCCTGGTCGAGCTGGACGGCGACGTAAACGGCCACAAGTTCAGCGTGTCCGGCGAGGGCGA
GGGCGATGCCACCTACGGCAAGCTGACCCTGAAGTTCATCTGCACCACCGGCAAGCTGCCCGTG
CCCTGGCCCACCCTCGTGACCACCCTGACCTACGGCGTGCAGTGCTTCAGCCGCTACCCCGACC
ACATGAAGCAGCACGACTTCTTCAAGTCCGCCATGCCCGAAGGCTACGTCCAGGAGCGCACCAT
CTTCTTCAAGGACGACGGCAACTACAAGACCCGCGCCGAGGTGAAGTTCGAGGGCGACACCCTG
GTGAACCGCATCGAGCTGAAGGGCATCGACTTCAAGGAGGACGGCAACATCCTGGGGCACAAGC
TGGAGTACAACTACAACAGCCACAACGTCTATATCATGGCCGACAAGCAGAAGAACGGCATCAA
GGTGAACTTCAAGATCCGCCACAACATCGAGGACGGCAGCGTGCAGCTCGCCGACCACTACCAG
CAGAACACCCCCATCGGCGACGGCCCCGTGCTGCTGCCCGACAACCACTACCTGAGCACCCAGT
CCGCCCTGAGCAAAGACCCCAACGAGAAGCGCGATCACATGGTCCTGCTGGAGTTCGTGACCGC
CGCCGGGATCACTCTCGGCATGGACGAGCTGTACAAGTAAcctgcaggcgccggcgGTGTCCCC
AGAGAGCCCGCACCAGTGCCATCTGCTTGGCCGATTTCTCCACCTTGACCACGCGTCTTGTGTC
TCTCCGCTCTGAGGTGACTGGTTGGGTGGAGATATGGGAAAAGCAGGACCCGCAGCTGCGTCCG
CCTCCCCTGCATGAGCTCGCTGATCAGCCTCGACTGTGCCTTCTAGTTGCCAGCCATCTGTTGT
TTGCCCCTCCCCCGTGCCTTCCTTGACCCTGGAAGGTGCCACTCCCACTGTCCTTTCCTAATAA
AATGAGGAAATTGCATCGCATTGTCTGAGTAGGTGTCATTCTATTCTGGGGGGTGGGGTGGGGC
AGGACAGCAAGGGGGAGGATTGGGAAGACAATAGCAGGCATGCTGGGGATGCGGTGGGCTCTAT
GGAAGCTTGAATTCAGCTGACGTGCCTCGGACCGCTAGGAACCCCTAGTGATGGAGTTGGCCAC
TCCCTCTCTGCGCGCTCGCTCGCTCACTGAGGCCGGGCGACCAAAGGTCGCCCGACGCCCGGGC
TTTGCCCGGGCGGCCTCAGTGAGCGAGCGAGCGCGCAG
Exemplary pITR.CAG.miR5i_miR6u-16_EGFP Construct sequence
(SEQ ID NO: 196)
CTGCGCGCTCGCTCGCTCACTGAGGCCGCCCGGGCGTCGGGCGACCTTTGGTCGCCCGGCCTCA
GTGAGCGAGCGAGCGCGCAGAGAGGGAGTGGCCAACTCCATCACTAGGGGTTCCTGCGGCCGCA
CGCGTGACATTGATTATTGACTAGTTATTAATAGTAATCAATTACGGGGTCATTAGTTCATAGC
CCATATATGGAGTTCCGCGTTACATAACTTACGGTAAATGGCCCGCCTGGCTGACCGCCCAACG
ACCCCCGCCCATTGACGTCAATAATGACGTATGTTCCCATAGTAACGCCAATAGGGACTTTCCA
TTGACGTCAATGGGTGGACTATTTACGGTAAACTGCCCACTTGGCAGTACATCAAGTGTATCAT
ATGCCAAGTACGCCCCCTATTGACGTCAATGACGGTAAATGGCCCGCCTGGCATTATGCCCAGT
ACATGACCTTATGGGACTTTCCTACTTGGCAGTACATCTACGTATTAGTCATCGCTATTACCAT
GGGTCGAGGTGAGCCCCACGTTCTGCTTCACTCTCCCCATCTCCCCCCCCTCCCCACCCCCAAT
TTTGTATTTATTTATTTTTTAATTATTTTGTGCAGCGATGGGGGCGGGGGGGGGGGGGGCGCGC
GCCAGGCGGGGCGGGGCGGGGCGAGGGGCGGGGCGGGGCGAGGCGGAGAGGTGCGGCGGCAGCC
AATCAGAGCGGCGCGCTCCGAAAGTTTCCTTTTATGGCGAGGCGGCGGCGGCGGCGGCCCTATA
AAAAGCGAAGCGCGCGGCGGGCGGGAGTCGCTGCGTTGCCTTCGCCCCGTGCCCCGCTCCGCGC
CGCCTCGCGCCGCCCGCCCCGGCTCTGACTGACCGCGTTACTCCCACAGGTGAGCGGGCGGGAC
GGCCCTTCTCCTCCGGGCTGTAATTAGCGCTTGGTTTAATGACGGCTCGTTTCTTTTCTGTGGC
TGCGTGAAAGCCTTAAAGGGCTCCGGGAGGGCCCTTTGTGCGGGGGGGAGCGGCTCGGGGGGTG
CGTGCGTGTGTGTGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGCGGCCGGGGGCGG
TGCCCCGCGGTGCGGGGGGGCTGCGAGGGGAACAAAGGCTGCGTGCGGGGTGTGTGCGTGGGGG
GGTGAGCAGGGGGTGTGGGCGCGGCGGTCGGGCTGTAACCCCCCCCTGCACCCCCCTCCCCGAG
TTGCTGAGCACGGCCCGGCTTCGGGTGCGGGGCTCCGTGCGGGGCGTGGCGCGGGGCTCGCCGT
GCCGGGCGGGGGGTGGCGGCAGGTGGGGGTGCCGGGCGGGGCGGGGCCGCCTCGGGCCGGGGAG
GGCTCGGGGGAGGGGCGCGGCGGCCCCCGGAGCGCCGGCGCATAGCTCTTATGATAGCAATGTC
AGCAGTGCCTTTCTCCACCTTGACCACGCGTGTTAAGATTCTAAAATTATCTCGTGTGGTAAGT
GGTGGAGAGAAGTAAGGTTGACCATACTCTACAGTTGTGTCTAGAGCCTCTGCTAACCATGTTC
ATGCCTTCTTCTTTTTCCTACAGCTCCTGGGCAACGTGCTGGTTATTGTGACCGGTCGCTAGCC
ACCATGGTGAGCAAGGGCGAGGAGCTGTTCACCGGGGTGGTGCCCATCCTGGTCGAGCTGGACG
GCGACGTAAACGGCCACAAGTTCAGCGTGTCCGGCGAGGGCGAGGGCGATGCCACCTACGGCAA
GCTGACCCTGAAGTTCATCTGCACCACCGGCAAGCTGCCCGTGCCCTGGCCCACCCTCGTGACC
ACCCTGACCTACGGCGTGCAGTGCTTCAGCCGCTACCCCGACCACATGAAGCAGCACGACTTCT
TCAAGTCCGCCATGCCCGAAGGCTACGTCCAGGAGCGCACCATCTTCTTCAAGGACGACGGCAA
CTACAAGACCCGCGCCGAGGTGAAGTTCGAGGGCGACACCCTGGTGAACCGCATCGAGCTGAAG
GGCATCGACTTCAAGGAGGACGGCAACATCCTGGGGCACAAGCTGGAGTACAACTACAACAGCC
ACAACGTCTATATCATGGCCGACAAGCAGAAGAACGGCATCAAGGTGAACTTCAAGATCCGCCA
CAACATCGAGGACGGCAGCGTGCAGCTCGCCGACCACTACCAGCAGAACACCCCCATCGGCGAC
GGCCCCGTGCTGCTGCCCGACAACCACTACCTGAGCACCCAGTCCGCCCTGAGCAAAGACCCCA
ACGAGAAGCGCGATCACATGGTCCTGCTGGAGTTCGTGACCGCCGCCGGGATCACTCTCGGCAT
GGACGAGCTGTACAAGTAAcctgcaggcgccggcgCATAGCTCTTATGATAGCAATGTCAGCAG
TGCCTATACTGTCATAGTAGTACCAGGTTAAGATTCTAAAATTATCATGGTACTAATGTTGGCA
GTGTAAGTAAGGTTGACCATACTCTACAGTTGTGGAGCTCGCTGATCAGCCTCGACTGTGCCTT
CTAGTTGCCAGCCATCTGTTGTTTGCCCCTCCCCCGTGCCTTCCTTGACCCTGGAAGGTGCCAC
TCCCACTGTCCTTTCCTAATAAAATGAGGAAATTGCATCGCATTGTCTGAGTAGGTGTCATTCT
ATTCTGGGGGGTGGGGTGGGGCAGGACAGCAAGGGGGAGGATTGGGAAGACAATAGCAGGCATG
CTGGGGATGCGGTGGGCTCTATGGAAGCTTGAATTCAGCTGACGTGCCTCGGACCGCTAGGAAC
CCCTAGTGATGGAGTTGGCCACTCCCTCTCTGCGCGCTCGCTCGCTCACTGAGGCCGGGCGACC
AAAGGTCGCCCGACGCCCGGGCTTTGCCCGGGCGGCCTCAGTGAGCGAGCGAGCGCGCAG
Exemplary pITR.CAG.miR5i_miR6u-26_EGFP Construct sequence
(SEQ ID NO: 197)
CTGCGCGCTCGCTCGCTCACTGAGGCCGCCCGGGCGTCGGGCGACCTTTGGTCGCCCGGCCTCA
GTGAGCGAGCGAGCGCGCAGAGAGGGAGTGGCCAACTCCATCACTAGGGGTTCCTGCGGCCGCA
CGCGTGACATTGATTATTGACTAGTTATTAATAGTAATCAATTACGGGGTCATTAGTTCATAGC
CCATATATGGAGTTCCGCGTTACATAACTTACGGTAAATGGCCCGCCTGGCTGACCGCCCAACG
ACCCCCGCCCATTGACGTCAATAATGACGTATGTTCCCATAGTAACGCCAATAGGGACTTTCCA
TTGACGTCAATGGGTGGACTATTTACGGTAAACTGCCCACTTGGCAGTACATCAAGTGTATCAT
ATGCCAAGTACGCCCCCTATTGACGTCAATGACGGTAAATGGCCCGCCTGGCATTATGCCCAGT
ACATGACCTTATGGGACTTTCCTACTTGGCAGTACATCTACGTATTAGTCATCGCTATTACCAT
GGGTCGAGGTGAGCCCCACGTTCTGCTTCACTCTCCCCATCTCCCCCCCCTCCCCACCCCCAAT
TTTGTATTTATTTATTTTTTAATTATTTTGTGCAGCGATGGGGGCGGGGGGGGGGGGGGCGCGC
GCCAGGCGGGGCGGGGCGGGGCGAGGGGCGGGGCGGGGCGAGGCGGAGAGGTGCGGCGGCAGCC
AATCAGAGCGGCGCGCTCCGAAAGTTTCCTTTTATGGCGAGGCGGCGGCGGCGGCGGCCCTATA
AAAAGCGAAGCGCGCGGCGGGCGGGAGTCGCTGCGTTGCCTTCGCCCCGTGCCCCGCTCCGCGC
CGCCTCGCGCCGCCCGCCCCGGCTCTGACTGACCGCGTTACTCCCACAGGTGAGCGGGCGGGAC
GGCCCTTCTCCTCCGGGCTGTAATTAGCGCTTGGTTTAATGACGGCTCGTTTCTTTTCTGTGGC
TGCGTGAAAGCCTTAAAGGGCTCCGGGAGGGCCCTTTGTGCGGGGGGGAGCGGCTCGGGGGGTG
CGTGCGTGTGTGTGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGCGGCCGGGGGCGG
TGCCCCGCGGTGCGGGGGGGCTGCGAGGGGAACAAAGGCTGCGTGCGGGGTGTGTGCGTGGGGG
GGTGAGCAGGGGGTGTGGGCGCGGCGGTCGGGCTGTAACCCCCCCCTGCACCCCCCTCCCCGAG
TTGCTGAGCACGGCCCGGCTTCGGGTGCGGGGCTCCGTGCGGGGCGTGGCGCGGGGCTCGCCGT
GCCGGGCGGGGGGTGGCGGCAGGTGGGGGTGCCGGGCGGGGCGGGGCCGCCTCGGGCCGGGGAG
GGCTCGGGGGAGGGGCGCGGCGGCCCCCGGAGCGCCGGCGGAGCAGGAGGACCAAGGCCCTGGC
GAAGGCCGTGGCCTCGTTCTCCACCTTGACCACGCGTTGTGCAGGTCCCAATGGGCGTGTGGTA
GAGGTGGAGCACGGGGACGCGGGCCTGGACGCCGGCATCCGGGCTCAGGACCCCCCTCTCTAGA
GCCTCTGCTAACCATGTTCATGCCTTCTTCTTTTTCCTACAGCTCCTGGGCAACGTGCTGGTTA
TTGTGACCGGTCGCTAGCCACCATGGTGAGCAAGGGCGAGGAGCTGTTCACCGGGGTGGTGCCC
ATCCTGGTCGAGCTGGACGGCGACGTAAACGGCCACAAGTTCAGCGTGTCCGGCGAGGGCGAGG
GCGATGCCACCTACGGCAAGCTGACCCTGAAGTTCATCTGCACCACCGGCAAGCTGCCCGTGCC
CTGGCCCACCCTCGTGACCACCCTGACCTACGGCGTGCAGTGCTTCAGCCGCTACCCCGACCAC
ATGAAGCAGCACGACTTCTTCAAGTCCGCCATGCCCGAAGGCTACGTCCAGGAGCGCACCATCT
TCTTCAAGGACGACGGCAACTACAAGACCCGCGCCGAGGTGAAGTTCGAGGGCGACACCCTGGT
GAACCGCATCGAGCTGAAGGGCATCGACTTCAAGGAGGACGGCAACATCCTGGGGCACAAGCTG
GAGTACAACTACAACAGCCACAACGTCTATATCATGGCCGACAAGCAGAAGAACGGCATCAAGG
TGAACTTCAAGATCCGCCACAACATCGAGGACGGCAGCGTGCAGCTCGCCGACCACTACCAGCA
GAACACCCCCATCGGCGACGGCCCCGTGCTGCTGCCCGACAACCACTACCTGAGCACCCAGTCC
GCCCTGAGCAAAGACCCCAACGAGAAGCGCGATCACATGGTCCTGCTGGAGTTCGTGACCGCCG
CCGGGATCACTCTCGGCATGGACGAGCTGTACAAGTAAcctgcaggcgccggcgGAGCAGGAGG
ACCAAGGCCCTGGCGAAGGCCGTGGCCTCGATACTGTCATAGTAGTACCAGTGTGCAGGTCCCA
ATGGCTGGTACTAATGTGACAGTCTCGGGGACGCGGGCCTGGACGCCGGCATCCGGGCTCAGGA
CCCCCCTCGAGCTCGCTGATCAGCCTCGACTGTGCCTTCTAGTTGCCAGCCATCTGTTGTTTGC
CCCTCCCCCGTGCCTTCCTTGACCCTGGAAGGTGCCACTCCCACTGTCCTTTCCTAATAAAATG
AGGAAATTGCATCGCATTGTCTGAGTAGGTGTCATTCTATTCTGGGGGGTGGGGTGGGGCAGGA
CAGCAAGGGGGAGGATTGGGAAGACAATAGCAGGCATGCTGGGGATGCGGTGGGCTCTATGGAA
GCTTGAATTCAGCTGACGTGCCTCGGACCGCTAGGAACCCCTAGTGATGGAGTTGGCCACTCCC
TCTCTGCGCGCTCGCTCGCTCACTGAGGCCGGGCGACCAAAGGTCGCCCGACGCCCGGGCTTTG
CCCGGGCGGCCTCAGTGAGCGAGCGAGCGCGCAG
Exemplary pITR.CAG.miR5i_miR6u-96_EGFP Construct sequence
(SEQ ID NO: 198)
CTGCGCGCTCGCTCGCTCACTGAGGCCGCCCGGGCGTCGGGCGACCTTTGGTCGCCCGGCCTCA
GTGAGCGAGCGAGCGCGCAGAGAGGGAGTGGCCAACTCCATCACTAGGGGTTCCTGCGGCCGCA
CGCGTGACATTGATTATTGACTAGTTATTAATAGTAATCAATTACGGGGTCATTAGTTCATAGC
CCATATATGGAGTTCCGCGTTACATAACTTACGGTAAATGGCCCGCCTGGCTGACCGCCCAACG
ACCCCCGCCCATTGACGTCAATAATGACGTATGTTCCCATAGTAACGCCAATAGGGACTTTCCA
TTGACGTCAATGGGTGGACTATTTACGGTAAACTGCCCACTTGGCAGTACATCAAGTGTATCAT
ATGCCAAGTACGCCCCCTATTGACGTCAATGACGGTAAATGGCCCGCCTGGCATTATGCCCAGT
ACATGACCTTATGGGACTTTCCTACTTGGCAGTACATCTACGTATTAGTCATCGCTATTACCAT
GGGTCGAGGTGAGCCCCACGTTCTGCTTCACTCTCCCCATCTCCCCCCCCTCCCCACCCCCAAT
TTTGTATTTATTTATTTTTTAATTATTTTGTGCAGCGATGGGGGCGGGGGGGGGGGGGGCGCGC
GCCAGGCGGGGCGGGGCGGGGCGAGGGGCGGGGCGGGGCGAGGCGGAGAGGTGCGGCGGCAGCC
AATCAGAGCGGCGCGCTCCGAAAGTTTCCTTTTATGGCGAGGCGGCGGCGGCGGCGGCCCTATA
AAAAGCGAAGCGCGCGGCGGGCGGGAGTCGCTGCGTTGCCTTCGCCCCGTGCCCCGCTCCGCGC
CGCCTCGCGCCGCCCGCCCCGGCTCTGACTGACCGCGTTACTCCCACAGGTGAGCGGGCGGGAC
GGCCCTTCTCCTCCGGGCTGTAATTAGCGCTTGGTTTAATGACGGCTCGTTTCTTTTCTGTGGC
TGCGTGAAAGCCTTAAAGGGCTCCGGGAGGGCCCTTTGTGCGGGGGGGAGCGGCTCGGGGGGTG
CGTGCGTGTGTGTGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGCGGCCGGGGGCGG
TGCCCCGCGGTGCGGGGGGGCTGCGAGGGGAACAAAGGCTGCGTGCGGGGTGTGTGCGTGGGGG
GGTGAGCAGGGGGTGTGGGCGCGGCGGTCGGGCTGTAACCCCCCCCTGCACCCCCCTCCCCGAG
TTGCTGAGCACGGCCCGGCTTCGGGTGCGGGGCTCCGTGCGGGGCGTGGCGCGGGGCTCGCCGT
GCCGGGCGGGGGGTGGCGGCAGGTGGGGGTGCCGGGCGGGGCGGGGCCGCCTCGGGCCGGGGAG
GGCTCGGGGGAGGGGCGCGGCGGCCCCCGGAGCGCCGGCGGTGTCCCCAGAGAGCCCGCACCAG
TGCCATCTGCTTGGCCGATTTCTCCACCTTGACCACGCGTCTTGTGTCTCTCCGCTCTGAGGTG
ACTGGTTGGGTGGAGATATGGGAAAAGCAGGACCCGCAGCTGCGTCCGCCTCCCCTGCATTCTA
GAGCCTCTGCTAACCATGTTCATGCCTTCTTCTTTTTCCTACAGCTCCTGGGCAACGTGCTGGT
TATTGTGACCGGTCGCTAGCCACCATGGTGAGCAAGGGCGAGGAGCTGTTCACCGGGGTGGTGC
CCATCCTGGTCGAGCTGGACGGCGACGTAAACGGCCACAAGTTCAGCGTGTCCGGCGAGGGCGA
GGGCGATGCCACCTACGGCAAGCTGACCCTGAAGTTCATCTGCACCACCGGCAAGCTGCCCGTG
CCCTGGCCCACCCTCGTGACCACCCTGACCTACGGCGTGCAGTGCTTCAGCCGCTACCCCGACC
ACATGAAGCAGCACGACTTCTTCAAGTCCGCCATGCCCGAAGGCTACGTCCAGGAGCGCACCAT
CTTCTTCAAGGACGACGGCAACTACAAGACCCGCGCCGAGGTGAAGTTCGAGGGCGACACCCTG
GTGAACCGCATCGAGCTGAAGGGCATCGACTTCAAGGAGGACGGCAACATCCTGGGGCACAAGC
TGGAGTACAACTACAACAGCCACAACGTCTATATCATGGCCGACAAGCAGAAGAACGGCATCAA
GGTGAACTTCAAGATCCGCCACAACATCGAGGACGGCAGCGTGCAGCTCGCCGACCACTACCAG
CAGAACACCCCCATCGGCGACGGCCCCGTGCTGCTGCCCGACAACCACTACCTGAGCACCCAGT
CCGCCCTGAGCAAAGACCCCAACGAGAAGCGCGATCACATGGTCCTGCTGGAGTTCGTGACCGC
CGCCGGGATCACTCTCGGCATGGACGAGCTGTACAAGTAAcctgcaggcgccggcgGTGTCCCC
AGAGAGCCCGCACCAGTGCCATCTGCTTGGCCGATATACTGTCATAGTAGTACCAGCTTGTGTC
TCTCCGCTCTGAGCTGTCACTGCTTGACGGTAAATGGGAAAAGCAGGACCCGCAGCTGCGTCCG
CCTCCCCTGCATGAGCTCGCTGATCAGCCTCGACTGTGCCTTCTAGTTGCCAGCCATCTGTTGT
TTGCCCCTCCCCCGTGCCTTCCTTGACCCTGGAAGGTGCCACTCCCACTGTCCTTTCCTAATAA
AATGAGGAAATTGCATCGCATTGTCTGAGTAGGTGTCATTCTATTCTGGGGGGTGGGGTGGGGC
AGGACAGCAAGGGGGAGGATTGGGAAGACAATAGCAGGCATGCTGGGGATGCGGTGGGCTCTAT
GGAAGCTTGAATTCAGCTGACGTGCCTCGGACCGCTAGGAACCCCTAGTGATGGAGTTGGCCAC
TCCCTCTCTGCGCGCTCGCTCGCTCACTGAGGCCGGGCGACCAAAGGTCGCCCGACGCCCGGGC
TTTGCCCGGGCGGCCTCAGTGAGCGAGCGAGCGCGCAG
Exemplary pITR.CAG.miR6-16_EGFP Construct sequence
(SEQ ID NO: 199)
CTGCGCGCTCGCTCGCTCACTGAGGCCGCCCGGGCGTCGGGCGACCTTTGGTCGCCCGGCCTCA
GTGAGCGAGCGAGCGCGCAGAGAGGGAGTGGCCAACTCCATCACTAGGGGTTCCTGCGGCCGCA
CGCGTGACATTGATTATTGACTAGTTATTAATAGTAATCAATTACGGGGTCATTAGTTCATAGC
CCATATATGGAGTTCCGCGTTACATAACTTACGGTAAATGGCCCGCCTGGCTGACCGCCCAACG
ACCCCCGCCCATTGACGTCAATAATGACGTATGTTCCCATAGTAACGCCAATAGGGACTTTCCA
TTGACGTCAATGGGTGGACTATTTACGGTAAACTGCCCACTTGGCAGTACATCAAGTGTATCAT
ATGCCAAGTACGCCCCCTATTGACGTCAATGACGGTAAATGGCCCGCCTGGCATTATGCCCAGT
ACATGACCTTATGGGACTTTCCTACTTGGCAGTACATCTACGTATTAGTCATCGCTATTACCAT
GGGTCGAGGTGAGCCCCACGTTCTGCTTCACTCTCCCCATCTCCCCCCCCTCCCCACCCCCAAT
TTTGTATTTATTTATTTTTTAATTATTTTGTGCAGCGATGGGGGCGGGGGGGGGGGGGGCGCGC
GCCAGGCGGGGCGGGGCGGGGCGAGGGGCGGGGCGGGGCGAGGCGGAGAGGTGCGGCGGCAGCC
AATCAGAGCGGCGCGCTCCGAAAGTTTCCTTTTATGGCGAGGCGGCGGCGGCGGCGGCCCTATA
AAAAGCGAAGCGCGCGGCGGGCGGGAGTCGCTGCGTTGCCTTCGCCCCGTGCCCCGCTCCGCGC
CGCCTCGCGCCGCCCGCCCCGGCTCTGACTGACCGCGTTACTCCCACAGGTGAGCGGGCGGGAC
GGCCCTTCTCCTCCGGGCTGTAATTAGCGCTTGGTTTAATGACGGCTCGTTTCTTTTCTGTGGC
TGCGTGAAAGCCTTAAAGGGCTCCGGGAGGGCCCTTTGTGCGGGGGGGAGCGGCTCGGGGGGTG
CGTGCGTGTGTGTGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGCGGCCGGGGGCGG
TGCCCCGCGGTGCGGGGGGGCTGCGAGGGGAACAAAGGCTGCGTGCGGGGTGTGTGCGTGGGGG
GGTGAGCAGGGGGTGTGGGCGCGGCGGTCGGGCTGTAACCCCCCCCTGCACCCCCCTCCCCGAG
TTGCTGAGCACGGCCCGGCTTCGGGTGCGGGGCTCCGTGCGGGGCGTGGCGCGGGGCTCGCCGT
GCCGGGCGGGGGGTGGCGGCAGGTGGGGGTGCCGGGCGGGGCGGGGCCGCCTCGGGCCGGGGAG
GGCTCGGGGGAGGGGCGCGGCGGCCCCCGGAGCGCCGGCGCATAGCTCTTATGATAGCAATGTC
AGGAGTGCCTATACTGTCATAGTAGTACGAGGTTAAGATTCTAAAATTATCATGGTACTAATGT
TGGCAGTGTAAGTAAGGTTGACCATACTCTACAGTTGTGTCTAGAGCCTCTGCTAACCATGTTC
ATGCCTTCTTCTTTTTCCTACAGCTCCTGGGCAACGTGCTGGTTATTGTGACCGGTCGCTAGCC
ACCATGGTGAGCAAGGGCGAGGAGCTGTTCACCGGGGTGGTGCCCATCCTGGTCGAGCTGGACG
GCGACGTAAACGGCCACAAGTTCAGCGTGTCCGGCGAGGGCGAGGGCGATGCCACCTACGGCAA
GCTGACCCTGAAGTTCATCTGCACCACCGGCAAGCTGCCCGTGCCCTGGCCCACCCTCGTGACC
ACCCTGACCTACGGCGTGCAGTGCTTCAGCCGCTACCCCGACCACATGAAGCAGCACGACTTCT
TCAAGTCCGCCATGCCCGAAGGCTACGTCCAGGAGCGCACCATCTTCTTCAAGGACGACGGCAA
CTACAAGACCCGCGCCGAGGTGAAGTTCGAGGGCGACACCCTGGTGAACCGCATCGAGCTGAAG
GGCATCGACTTCAAGGAGGACGGCAACATCCTGGGGCACAAGCTGGAGTACAACTACAACAGCC
ACAACGTCTATATCATGGCCGACAAGCAGAAGAACGGCATCAAGGTGAACTTCAAGATCCGCCA
CAACATCGAGGACGGCAGCGTGCAGCTCGCCGACCACTACCAGCAGAACACCCCCATCGGCGAC
GGCCCCGTGCTGCTGCCCGACAACCACTACCTGAGCACCCAGTCCGCCCTGAGCAAAGACCCCA
ACGAGAAGCGCGATCACATGGTCCTGCTGGAGTTCGTGACCGCCGCCGGGATCACTCTCGGCAT
GGACGAGCTGTACAAGTAAcctgcaggcgccggcgCATAGCTCTTATGATAGCAATGTCAGCAG
TGCCTATACTGTCATAGTAGTACCAGGTTAAGATTCTAAAATTATCATGGTACTAATGTTGGCA
GTGTAAGTAAGGTTGACCATACTCTACAGTTGTGGAGCTCGCTGATCAGCCTCGACTGTGCCTT
CTAGTTGCCAGCCATCTGTTGTTTGCCCCTCCCCCGTGCCTTCCTTGACCCTGGAAGGTGCCAC
TCCCACTGTCCTTTCCTAATAAAATGAGGAAATTGCATCGCATTGTCTGAGTAGGTGTCATTCT
ATTCTGGGGGGTGGGGTGGGGCAGGACAGCAAGGGGGAGGATTGGGAAGACAATAGCAGGCATG
CTGGGGATGCGGTGGGCTCTATGGAAGCTTGAATTCAGCTGACGTGCCTCGGACCGCTAGGAAC
CCCTAGTGATGGAGTTGGCCACTCCCTCTCTGCGCGCTCGCTCGCTCACTGAGGCCGGGCGACC
AAAGGTCGCCCGACGCCCGGGCTTTGCCCGGGCGGCCTCAGTGAGCGAGCGAGCGCGCAG
Exemplary pITR.CAG.miR6-26_EGFP Construct sequence
(SEQ ID NO: 200)
CTGCGCGCTCGCTCGCTCACTGAGGCCGCCCGGGCGTCGGGCGACCTTTGGTCGCCCGGCCTCA
GTGAGCGAGCGAGCGCGCAGAGAGGGAGTGGCCAACTCCATCACTAGGGGTTCCTGCGGCCGCA
CGCGTGACATTGATTATTGACTAGTTATTAATAGTAATCAATTACGGGGTCATTAGTTCATAGC
CCATATATGGAGTTCCGCGTTACATAACTTACGGTAAATGGCCCGCCTGGCTGACCGCCCAACG
ACCCCCGCCCATTGACGTCAATAATGACGTATGTTCCCATAGTAACGCCAATAGGGACTTTCCA
TTGACGTCAATGGGTGGACTATTTACGGTAAACTGCCCACTTGGCAGTACATCAAGTGTATCAT
ATGCCAAGTACGCCCCCTATTGACGTCAATGACGGTAAATGGCCCGCCTGGCATTATGCCCAGT
ACATGACCTTATGGGACTTTCCTACTTGGCAGTACATCTACGTATTAGTCATCGCTATTACCAT
GGGTCGAGGTGAGCCCCACGTTCTGCTTCACTCTCCCCATCTCCCCCCCCTCCCCACCCCCAAT
TTTGTATTTATTTATTTTTTAATTATTTTGTGCAGCGATGGGGGCGGGGGGGGGGGGGGCGCGC
GCCAGGCGGGGCGGGGCGGGGCGAGGGGCGGGGCGGGGCGAGGCGGAGAGGTGCGGCGGCAGCC
AATCAGAGCGGCGCGCTCCGAAAGTTTCCTTTTATGGCGAGGCGGCGGCGGCGGCGGCCCTATA
AAAAGCGAAGCGCGCGGCGGGCGGGAGTCGCTGCGTTGCCTTCGCCCCGTGCCCCGCTCCGCGC
CGCCTCGCGCCGCCCGCCCCGGCTCTGACTGACCGCGTTACTCCCACAGGTGAGCGGGCGGGAC
GGCCCTTCTCCTCCGGGCTGTAATTAGCGCTTGGTTTAATGACGGCTCGTTTCTTTTCTGTGGC
TGCGTGAAAGCCTTAAAGGGCTCCGGGAGGGCCCTTTGTGCGGGGGGGAGCGGCTCGGGGGGTG
CGTGCGTGTGTGTGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGCGGCCGGGGGCGG
TGCCCCGCGGTGCGGGGGGGCTGCGAGGGGAACAAAGGCTGCGTGCGGGGTGTGTGCGTGGGGG
GGTGAGCAGGGGGTGTGGGCGCGGCGGTCGGGCTGTAACCCCCCCCTGCACCCCCCTCCCCGAG
TTGCTGAGCACGGCCCGGCTTCGGGTGCGGGGCTCCGTGCGGGGCGTGGCGCGGGGCTCGCCGT
GCCGGGCGGGGGGTGGCGGCAGGTGGGGGTGCCGGGCGGGGCGGGGCCGCCTCGGGCCGGGGAG
GGCTCGGGGGAGGGGCGCGGCGGCCCCCGGAGCGCCGGCGGAGCAGGAGGACCAAGGCCCTGGC
GAAGGCCGTGGCCTCGATACTGTCATAGTAGTACCAGTGTGCAGGTCCCAATGGCTGGTACTAA
TGTGACAGTCTCGGGGACGCGGGCCTGGACGCCGGCATCCGGGCTCAGGACCCCCCTCTCTAGA
GCCTCTGCTAACCATGTTCATGCCTTCTTCTTTTTCCTACAGCTCCTGGGCAACGTGCTGGTTA
TTGTGACCGGTCGCTAGCCACCATGGTGAGCAAGGGCGAGGAGCTGTTCACCGGGGTGGTGCCC
ATCCTGGTCGAGCTGGACGGCGACGTAAACGGCCACAAGTTCAGCGTGTCCGGCGAGGGCGAGG
GCGATGCCACCTACGGCAAGCTGACCCTGAAGTTCATCTGCACCACCGGCAAGCTGCCCGTGCC
CTGGCCCACCCTCGTGACCACCCTGACCTACGGCGTGCAGTGCTTCAGCCGCTACCCCGACCAC
ATGAAGCAGCACGACTTCTTCAAGTCCGCCATGCCCGAAGGCTACGTCCAGGAGCGCACCATCT
TCTTCAAGGACGACGGCAACTACAAGACCCGCGCCGAGGTGAAGTTCGAGGGCGACACCCTGGT
GAACCGCATCGAGCTGAAGGGCATCGACTTCAAGGAGGACGGCAACATCCTGGGGCACAAGCTG
GAGTACAACTACAACAGCCACAACGTCTATATCATGGCCGACAAGCAGAAGAACGGCATCAAGG
TGAACTTCAAGATCCGCCACAACATCGAGGACGGCAGCGTGCAGCTCGCCGACCACTACCAGCA
GAACACCCCCATCGGCGACGGCCCCGTGCTGCTGCCCGACAACCACTACCTGAGCACCCAGTCC
GCCCTGAGCAAAGACCCCAACGAGAAGCGCGATCACATGGTCCTGCTGGAGTTCGTGACCGCCG
CCGGGATCACTCTCGGCATGGACGAGCTGTACAAGTAAcctgcaggcgccggcgGAGCAGGAGG
ACCAAGGCCCTGGCGAAGGCCGTGGCCTCGATACTGTCATAGTAGTACCAGTGTGCAGGTCCCA
ATGGCTGGTACTAATGTGACAGTCTCGGGGACGCGGGCCTGGACGCCGGCATCCGGGCTCAGGA
CCCCCCTCGAGCTCGCTGATCAGCCTCGACTGTGCCTTCTAGTTGCCAGCCATCTGTTGTTTGC
CCCTCCCCCGTGCCTTCCTTGACCCTGGAAGGTGCCACTCCCACTGTCCTTTCCTAATAAAATG
AGGAAATTGCATCGCATTGTCTGAGTAGGTGTCATTCTATTCTGGGGGGTGGGGTGGGGCAGGA
CAGCAAGGGGGAGGATTGGGAAGACAATAGCAGGCATGCTGGGGATGCGGTGGGCTCTATGGAA
GCTTGAATTCAGCTGACGTGCCTCGGACCGCTAGGAACCCCTAGTGATGGAGTTGGCCACTCCC
TCTCTGCGCGCTCGCTCGCTCACTGAGGCCGGGCGACCAAAGGTCGCCCGACGCCCGGGCTTTG
CCCGGGCGGCCTCAGTGAGCGAGCGAGCGCGCAG
Exemplary pITR.CAG.miR6-96_EGFP Construct sequence
(SEQ ID NO: 201)
CTGCGCGCTCGCTCGCTCACTGAGGCCGCCCGGGCGTCGGGCGACCTTTGGTCGCCCGGCCTCA
GTGAGCGAGCGAGCGCGCAGAGAGGGAGTGGCCAACTCCATCACTAGGGGTTCCTGCGGCCGCA
CGCGTGACATTGATTATTGACTAGTTATTAATAGTAATCAATTACGGGGTCATTAGTTCATAGC
CCATATATGGAGTTCCGCGTTACATAACTTACGGTAAATGGCCCGCCTGGCTGACCGCCCAACG
ACCCCCGCCCATTGACGTCAATAATGACGTATGTTCCCATAGTAACGCCAATAGGGACTTTCCA
TTGACGTCAATGGGTGGACTATTTACGGTAAACTGCCCACTTGGCAGTACATCAAGTGTATCAT
ATGCCAAGTACGCCCCCTATTGACGTCAATGACGGTAAATGGCCCGCCTGGCATTATGCCCAGT
ACATGACCTTATGGGACTTTCCTACTTGGCAGTACATCTACGTATTAGTCATCGCTATTACCAT
GGGTCGAGGTGAGCCCCACGTTCTGCTTCACTCTCCCCATCTCCCCCCCCTCCCCACCCCCAAT
TTTGTATTTATTTATTTTTTAATTATTTTGTGCAGCGATGGGGGCGGGGGGGGGGGGGGCGCGC
GCCAGGCGGGGCGGGGCGGGGCGAGGGGCGGGGCGGGGCGAGGCGGAGAGGTGCGGCGGCAGCC
AATCAGAGCGGCGCGCTCCGAAAGTTTCCTTTTATGGCGAGGCGGCGGCGGCGGCGGCCCTATA
AAAAGCGAAGCGCGCGGCGGGCGGGAGTCGCTGCGTTGCCTTCGCCCCGTGCCCCGCTCCGCGC
CGCCTCGCGCCGCCCGCCCCGGCTCTGACTGACCGCGTTACTCCCACAGGTGAGCGGGCGGGAC
GGCCCTTCTCCTCCGGGCTGTAATTAGCGCTTGGTTTAATGACGGCTCGTTTCTTTTCTGTGGC
TGCGTGAAAGCCTTAAAGGGCTCCGGGAGGGCCCTTTGTGCGGGGGGGAGCGGCTCGGGGGGTG
CGTGCGTGTGTGTGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGCGGCCGGGGGCGG
TGCCCCGCGGTGCGGGGGGGCTGCGAGGGGAACAAAGGCTGCGTGCGGGGTGTGTGCGTGGGGG
GGTGAGCAGGGGGTGTGGGCGCGGCGGTCGGGCTGTAACCCCCCCCTGCACCCCCCTCCCCGAG
TTGCTGAGCACGGCCCGGCTTCGGGTGCGGGGCTCCGTGCGGGGCGTGGCGCGGGGCTCGCCGT
GCCGGGCGGGGGGTGGCGGCAGGTGGGGGTGCCGGGCGGGGCGGGGCCGCCTCGGGCCGGGGAG
GGCTCGGGGGAGGGGCGCGGCGGCCCCCGGAGCGCCGGCGGTGTCCCCAGAGAGCCCGCACCAG
TGCCATCTGCTTGGCCGATATACTGTCATAGTAGTACCAGCTTGTGTCTCTCCGCTCTGAGCTG
TCACTGCTTGACGGTAAATGGGAAAAGCAGGACCCGCAGCTGCGTCCGCCTCCCCTGCATTCTA
GAGCCTCTGCTAACCATGTTCATGCCTTCTTCTTTTTCCTACAGCTCCTGGGCAACGTGCTGGT
TATTGTGACCGGTCGCTAGCCACCATGGTGAGCAAGGGCGAGGAGCTGTTCACCGGGGTGGTGC
CCATCCTGGTCGAGCTGGACGGCGACGTAAACGGCCACAAGTTCAGCGTGTCCGGCGAGGGCGA
GGGCGATGCCACCTACGGCAAGCTGACCCTGAAGTTCATCTGCACCACCGGCAAGCTGCCCGTG
CCCTGGCCCACCCTCGTGACCACCCTGACCTACGGCGTGCAGTGCTTCAGCCGCTACCCCGACC
ACATGAAGCAGCACGACTTCTTCAAGTCCGCCATGCCCGAAGGCTACGTCCAGGAGCGCACCAT
CTTCTTCAAGGACGACGGCAACTACAAGACCCGCGCCGAGGTGAAGTTCGAGGGCGACACCCTG
GTGAACCGCATCGAGCTGAAGGGCATCGACTTCAAGGAGGACGGCAACATCCTGGGGCACAAGC
TGGAGTACAACTACAACAGCCACAACGTCTATATCATGGCCGACAAGCAGAAGAACGGCATCAA
GGTGAACTTCAAGATCCGCCACAACATCGAGGACGGCAGCGTGCAGCTCGCCGACCACTACCAG
CAGAACACCCCCATCGGCGACGGCCCCGTGCTGCTGCCCGACAACCACTACCTGAGCACCCAGT
CCGCCCTGAGCAAAGACCCCAACGAGAAGCGCGATCACATGGTCCTGCTGGAGTTCGTGACCGC
CGCCGGGATCACTCTCGGCATGGACGAGCTGTACAAGTAAcctgcaggcgccggcgGTGTCCCC
AGAGAGCCCGCACCAGTGCCATCTGCTTGGCCGATATACTGTCATAGTAGTACCAGCTTGTGTC
TCTCCGCTCTGAGCTGTCACTGCTTGACGGTAAATGGGAAAAGCAGGACCCGCAGCTGCGTCCG
CCTCCCCTGCATGAGCTCGCTGATCAGCCTCGACTGTGCCTTCTAGTTGCCAGCCATCTGTTGT
TTGCCCCTCCCCCGTGCCTTCCTTGACCCTGGAAGGTGCCACTCCCACTGTCCTTTCCTAATAA
AATGAGGAAATTGCATCGCATTGTCTGAGTAGGTGTCATTCTATTCTGGGGGGTGGGGTGGGGC
AGGACAGCAAGGGGGAGGATTGGGAAGACAATAGCAGGCATGCTGGGGATGCGGTGGGCTCTAT
GGAAGCTTGAATTCAGCTGACGTGCCTCGGACCGCTAGGAACCCCTAGTGATGGAGTTGGCCAC
TCCCTCTCTGCGCGCTCGCTCGCTCACTGAGGCCGGGCGACCAAAGGTCGCCCGACGCCCGGGC
TTTGCCCGGGCGGCCTCAGTGAGCGAGCGAGCGCGCAG

Exemplary miRNA Construct Sequences (Part 2)

Exemplary pITR.CAG.miR1-155_EGFP
sequence
(SEQ ID NO: 202)
CTGCGCGCTCGCTCGCTCACTGAGGCCGCCCGGGCGTCGGGCGACCT
TTGGTCGCCCGGCCTCAGTGAGCGAGCGAGCGCGCAGAGAGGGAGTG
GCCAACTCCATCACTAGGGGTTCCTGCGGCCGCACGCGTGACATTGA
TTATTGACTAGTTATTAATAGTAATCAATTACGGGGTCATTAGTTCA
TAGCCCATATATGGAGTTCCGCGTTACATAACTTACGGTAAATGGCC
CGCCTGGCTGACCGCCCAACGACCCCCGCCCATTGACGTCAATAATG
ACGTATGTTCCCATAGTAACGCCAATAGGGACTTTCCATTGACGTCA
ATGGGTGGACTATTTACGGTAAACTGCCCACTTGGCAGTACATCAAG
TGTATCATATGCCAAGTACGCCCCCTATTGACGTCAATGACGGTAAA
TGGCCCGCCTGGCATTATGCCCAGTACATGACCTTATGGGACTTTCC
TACTTGGCAGTACATCTACGTATTAGTCATCGCTATTACCATGGGTC
GAGGTGAGCCCCACGTTCTGCTTCACTCTCCCCATCTCCCCCCCCTC
CCCACCCCCAATTTTGTATTTATTTATTTTTTAATTATTTTGTGCAG
CGATGGGGGCGGGGGGGGGGGGGGCGCGCGCCAGGCGGGGCGGGGCG
GGGCGAGGGGCGGGGCGGGGCGAGGCGGAGAGGTGCGGCGGCAGCCA
ATCAGAGCGGCGCGCTCCGAAAGTTTCCTTTTATGGCGAGGCGGCGG
CGGCGGCGGCCCTATAAAAAGCGAAGCGCGCGGCGGGCGGGAGTCGC
TGCGTTGCCTTCGCCCCGTGCCCCGCTCCGCGCCGCCTCGCGCCGCC
CGCCCCGGCTCTGACTGACCGCGTTACTCCCACAGGTGAGCGGGCGG
GACGGCCCTTCTCCTCCGGGCTGTAATTAGCGCTTGGTTTAATGACG
GCTCGTTTCTTTTCTGTGGCTGCGTGAAAGCCTTAAAGGGCTCCGGG
AGGGCCCTTTGTGCGGGGGGGAGCGGCTCGGGGGGTGCGTGCGTGTG
TGTGTGCGTGGGGAGCGCCGCGTGCGGCCCGCGCTGCCCGGCGGCTG
TGAGCGCTGCGGGCGCGGCGCGGGGCTTTGTGCGCTCCGCGTGTGCG
CGAGGGGAGCGCGGCCGGGGGCGGTGCCCCGCGGTGCGGGGGGGCTG
CGAGGGGAACAAAGGCTGCGTGCGGGGTGTGTGCGTGGGGGGGTGAG
CAGGGGGTGTGGGCGCGGCGGTCGGGCTGTAACCCCCCCCTGCACCC
CCCTCCCCGAGTTGCTGAGCACGGCCCGGCTTCGGGTGCGGGGCTCC
GTGCGGGGCGTGGCGCGGGGCTCGCCGTGCCGGGCGGGGGGTGGCGG
CAGGTGGGGGTGCCGGGCGGGGCGGGGCCGCCTCGGGCCGGGGAGGG
CTCGGGGGAGGGGCGCGGCGGCCCCCGGAGCGCCGGCGtcctctgag
tgctgaaggcttgctgtaggctgtatgctgTTGAGAATCCTGATGGA
GCgggtttttgcctccaactgacccGCTCATAGGATTCTCAAcagtg
tatgatgcctgttactagcattcacatggaTCTAGAGCCTCTGCTAA
CCATGTTCATGCCTTCTTCTTTTTCCTACAGCTCCTGGGCAACGTGC
TGGTTATTGTGACCGGTCGCTAGCCACCATGGTGAGCAAGGGCGAGG
AGCTGTTCACCGGGGTGGTGCCCATCCTGGTCGAGCTGGACGGCGAC
GTAAACGGCCACAAGTTCAGCGTGTCCGGCGAGGGCGAGGGCGATGC
CACCTACGGCAAGCTGACCCTGAAGTTCATCTGCACCACCGGCAAGC
TGCCCGTGCCCTGGCCCACCCTCGTGACCACCCTGACCTACGGCGTG
CAGTGCTTCAGCCGCTACCCCGACCACATGAAGCAGCACGACTTCTT
CAAGTCCGCCATGCCCGAAGGCTACGTCCAGGAGCGCACCATCTTCT
TCAAGGACGACGGCAACTACAAGACCCGCGCCGAGGTGAAGTTCGAG
GGCGACACCCTGGTGAACCGCATCGAGCTGAAGGGCATCGACTTCAA
GGAGGACGGCAACATCCTGGGGCACAAGCTGGAGTACAACTACAACA
GCCACAACGTCTATATCATGGCCGACAAGCAGAAGAACGGCATCAAG
GTGAACTTCAAGATCCGCCACAACATCGAGGACGGCAGCGTGCAGCT
CGCCGACCACTACCAGCAGAACACCCCCATCGGCGACGGCCCCGTGC
TGCTGCCCGACAACCACTACCTGAGCACCCAGTCCGCCCTGAGCAAA
GACCCCAACGAGAAGCGCGATCACATGGTCCTGCTGGAGTTCGTGAC
CGCCGCCGGGATCACTCTCGGCATGGACGAGCTGTACAAGTAAcctg
caggcgccggcgtcctctgagtgctgaaggcttgctgtaggctgtat
gctgTTGAGAATCCTGATGGAGCgggtttttgcctccaactgacccG
CTCATAGGATTCTCAAcagtgtatgatgcctgttactagcattcaca
tggaGAGCTCGCTGATCAGCCTCGACTGTGCCTTCTAGTTGCCAGCC
ATCTGTTGTTTGCCCCTCCCCCGTGCCTTCCTTGACCCTGGAAGGTG
CCACTCCCACTGTCCTTTCCTAATAAAATGAGGAAATTGCATCGCAT
TGTCTGAGTAGGTGTCATTCTATTCTGGGGGGTGGGGTGGGGCAGGA
CAGCAAGGGGGAGGATTGGGAAGACAATAGCAGGCATGCTGGGGATG
CGGTGGGCTCTATGGAAGCTTGAATTCAGCTGACGTGCCTCGGACCG
CTAGGAACCCCTAGTGATGGAGTTGGCCACTCCCTCTCTGCGCGCTC
GCTCGCTCACTGAGGCCGGGCGACCAAAGGTCGCCCGACGCCCGGGC
TTTGCCCGGGCGGCCTCAGTGAGCGAGCGAGCGCGCAG
Exemplary pITR.CAG.miR1i-155_EGFP
sequence
(SEQ ID NO: 203)
CTGCGCGCTCGCTCGCTCACTGAGGCCGCCCGGGCGTCGGGCGACCT
TTGGTCGCCCGGCCTCAGTGAGCGAGCGAGCGCGCAGAGAGGGAGTG
GCCAACTCCATCACTAGGGGTTCCTGCGGCCGCACGCGTGACATTGA
TTATTGACTAGTTATTAATAGTAATCAATTACGGGGTCATTAGTTCA
TAGCCCATATATGGAGTTCCGCGTTACATAACTTACGGTAAATGGCC
CGCCTGGCTGACCGCCCAACGACCCCCGCCCATTGACGTCAATAATG
ACGTATGTTCCCATAGTAACGCCAATAGGGACTTTCCATTGACGTCA
ATGGGTGGACTATTTACGGTAAACTGCCCACTTGGCAGTACATCAAG
TGTATCATATGCCAAGTACGCCCCCTATTGACGTCAATGACGGTAAA
TGGCCCGCCTGGCATTATGCCCAGTACATGACCTTATGGGACTTTCC
TACTTGGCAGTACATCTACGTATTAGTCATCGCTATTACCATGGGTC
GAGGTGAGCCCCACGTTCTGCTTCACTCTCCCCATCTCCCCCCCCTC
CCCACCCCCAATTTTGTATTTATTTATTTTTTAATTATTTTGTGCAG
CGATGGGGGCGGGGGGGGGGGGGGCGCGCGCCAGGCGGGGCGGGGCG
GGGCGAGGGGCGGGGCGGGGCGAGGCGGAGAGGTGCGGCGGCAGCCA
ATCAGAGCGGCGCGCTCCGAAAGTTTCCTTTTATGGCGAGGCGGCGG
CGGCGGCGGCCCTATAAAAAGCGAAGCGCGCGGCGGGCGGGAGTCGC
TGCGTTGCCTTCGCCCCGTGCCCCGCTCCGCGCCGCCTCGCGCCGCC
CGCCCCGGCTCTGACTGACCGCGTTACTCCCACAGGTGAGCGGGCGG
GACGGCCCTTCTCCTCCGGGCTGTAATTAGCGCTTGGTTTAATGACG
GCTCGTTTCTTTTCTGTGGCTGCGTGAAAGCCTTAAAGGGCTCCGGG
AGGGCCCTTTGTGCGGGGGGGAGCGGCTCGGGGGGTGCGTGCGTGTG
TGTGTGCGTGGGGAGCGCCGCGTGCGGCCCGCGCTGCCCGGCGGCTG
TGAGCGCTGCGGGCGCGGCGCGGGGCTTTGTGCGCTCCGCGTGTGCG
CGAGGGGAGCGCGGCCGGGGGCGGTGCCCCGCGGTGCGGGGGGGCTG
CGAGGGGAACAAAGGCTGCGTGCGGGGTGTGTGCGTGGGGGGGTGAG
CAGGGGGTGTGGGCGCGGCGGTCGGGCTGTAACCCCCCCCTGCACCC
CCCTCCCCGAGTTGCTGAGCACGGCCCGGCTTCGGGTGCGGGGCTCC
GTGCGGGGCGTGGCGCGGGGCTCGCCGTGCCGGGCGGGGGGTGGCGG
CAGGTGGGGGTGCCGGGCGGGGCGGGGCCGCCTCGGGCCGGGGAGGG
CTCGGGGGAGGGGCGCGGCGGCCCCCGGAGCGCCGGCGtcctctgag
tgctgaaggcttgctgtaggctgtatgctgTTGAGAATCCTGATGGA
GCgggtttttgcctccaactgacccGCTCATAGGATTCTCAAcagtg
tatgatgcctgttactagcattcacatggaTCTAGAGCCTCTGCTAA
CCATGTTCATGCCTTCTTCTTTTTCCTACAGCTCCTGGGCAACGTGC
TGGTTATTGTGACCGGTCGCTAGCCACCATGGTGAGCAAGGGCGAGG
AGCTGTTCACCGGGGTGGTGCCCATCCTGGTCGAGCTGGACGGCGAC
GTAAACGGCCACAAGTTCAGCGTGTCCGGCGAGGGCGAGGGCGATGC
CACCTACGGCAAGCTGACCCTGAAGTTCATCTGCACCACCGGCAAGC
TGCCCGTGCCCTGGCCCACCCTCGTGACCACCCTGACCTACGGCGTG
CAGTGCTTCAGCCGCTACCCCGACCACATGAAGCAGCACGACTTCTT
CAAGTCCGCCATGCCCGAAGGCTACGTCCAGGAGCGCACCATCTTCT
TCAAGGACGACGGCAACTACAAGACCCGCGCCGAGGTGAAGTTCGAG
GGCGACACCCTGGTGAACCGCATCGAGCTGAAGGGCATCGACTTCAA
GGAGGACGGCAACATCCTGGGGCACAAGCTGGAGTACAACTACAACA
GCCACAACGTCTATATCATGGCCGACAAGCAGAAGAACGGCATCAAG
GTGAACTTCAAGATCCGCCACAACATCGAGGACGGCAGCGTGCAGCT
CGCCGACCACTACCAGCAGAACACCCCCATCGGCGACGGCCCCGTGC
TGCTGCCCGACAACCACTACCTGAGCACCCAGTCCGCCCTGAGCAAA
GACCCCAACGAGAAGCGCGATCACATGGTCCTGCTGGAGTTCGTGAC
CGCCGCCGGGATCACTCTCGGCATGGACGAGCTGTACAAGTAAGAGC
TCGCTGATCAGCCTCGACTGTGCCTTCTAGTTGCCAGCCATCTGTTG
TTTGCCCCTCCCCCGTGCCTTCCTTGACCCTGGAAGGTGCCACTCCC
ACTGTCCTTTCCTAATAAAATGAGGAAATTGCATCGCATTGTCTGAG
TAGGTGTCATTCTATTCTGGGGGGTGGGGTGGGGCAGGACAGCAAGG
GGGAGGATTGGGAAGACAATAGCAGGCATGCTGGGGATGCGGTGGGC
TCTATGGAAGCTTGAATTCAGCTGACGTGCCTCGGACCGCTAGGAAC
CCCTAGTGATGGAGTTGGCCACTCCCTCTCTGCGCGCTCGCTCGCTC
ACTGAGGCCGGGCGACCAAAGGTCGCCCGACGCCCGGGCTTTGCCCG
GGCGGCCTCAGTGAGCGAGCGAGCGCGCAG
Exemplary pITR.CAG.miR1u-155_EGFP
sequence
(SEQ ID NO: 204)
CTGCGCGCTCGCTCGCTCACTGAGGCCGCCCGGGCGTCGGGCGACCT
TTGGTCGCCCGGCCTCAGTGAGCGAGCGAGCGCGCAGAGAGGGAGTG
GCCAACTCCATCACTAGGGGTTCCTGCGGCCGCACGCGTGACATTGA
TTATTGACTAGTTATTAATAGTAATCAATTACGGGGTCATTAGTTCA
TAGCCCATATATGGAGTTCCGCGTTACATAACTTACGGTAAATGGCC
CGCCTGGCTGACCGCCCAACGACCCCCGCCCATTGACGTCAATAATG
ACGTATGTTCCCATAGTAACGCCAATAGGGACTTTCCATTGACGTCA
ATGGGTGGACTATTTACGGTAAACTGCCCACTTGGCAGTACATCAAG
TGTATCATATGCCAAGTACGCCCCCTATTGACGTCAATGACGGTAAA
TGGCCCGCCTGGCATTATGCCCAGTACATGACCTTATGGGACTTTCC
TACTTGGCAGTACATCTACGTATTAGTCATCGCTATTACCATGGGTC
GAGGTGAGCCCCACGTTCTGCTTCACTCTCCCCATCTCCCCCCCCTC
CCCACCCCCAATTTTGTATTTATTTATTTTTTAATTATTTTGTGCAG
CGATGGGGGCGGGGGGGGGGGGGGCGCGCGCCAGGCGGGGCGGGGCG
GGGCGAGGGGCGGGGCGGGGCGAGGCGGAGAGGTGCGGCGGCAGCCA
ATCAGAGCGGCGCGCTCCGAAAGTTTCCTTTTATGGCGAGGCGGCGG
CGGCGGCGGCCCTATAAAAAGCGAAGCGCGCGGCGGGCGGGAGTCGC
TGCGTTGCCTTCGCCCCGTGCCCCGCTCCGCGCCGCCTCGCGCCGCC
CGCCCCGGCTCTGACTGACCGCGTTACTCCCACAGGTGAGCGGGCGG
GACGGCCCTTCTCCTCCGGGCTGTAATTAGCGCTTGGTTTAATGACG
GCTCGTTTCTTTTCTGTGGCTGCGTGAAAGCCTTAAAGGGCTCCGGG
AGGGCCCTTTGTGCGGGGGGGAGCGGCTCGGGGGGTGCGTGCGTGTG
TGTGTGCGTGGGGAGCGCCGCGTGCGGCCCGCGCTGCCCGGCGGCTG
TGAGCGCTGCGGGCGCGGCGCGGGGCTTTGTGCGCTCCGCGTGTGCG
CGAGGGGAGCGCGGCCGGGGGCGGTGCCCCGCGGTGCGGGGGGGCTG
CGAGGGGAACAAAGGCTGCGTGCGGGGTGTGTGCGTGGGGGGGTGAG
CAGGGGGTGTGGGCGCGGCGGTCGGGCTGTAACCCCCCCCTGCACCC
CCCTCCCCGAGTTGCTGAGCACGGCCCGGCTTCGGGTGCGGGGCTCC
GTGCGGGGCGTGGCGCGGGGCTCGCCGTGCCGGGCGGGGGGTGGCGG
CAGGTGGGGGTGCCGGGCGGGGCGGGGCCGCCTCGGGCCGGGGAGGG
CTCGGGGGAGGGGCGCGGCGGCCCCCGGAGCGCCGGCGGCTGTCGAG
GCGCGGCGAGCCGCAGCCATTGCCTTTTATGGTAATCGTGCGAGAGG
GCGCAGGGACTTCCTTTGTCCCAAATCTGTGCGGAGCCGAAATCTGG
GAGGCGCCGCCGCACCCCCTCTAGCGGGCGCGGGGCGAAGCGGTGCG
GCGCCGGCAGGAAGGAAATGGGCGGGGAGGGCCTTCGTGCGTCGCCG
CGCCGCCGTCCCCTTCTCCCTCTCCAGCCTCGGGGCTGTCCGCGGGG
GGACGGCTGCCTTCGGGGGGGACGGGGCAGGGCGGGGTTCGGCTTCT
GGCGTGTGACCGGCGGCTCTAGAGCCTCTGCTAACCATGTTCATGCC
TTCTTCTTTTTCCTACAGCTCCTGGGCAACGTGCTGGTTATTGTGAC
CGGTCGCTAGCCACCATGGTGAGCAAGGGCGAGGAGCTGTTCACCGG
GGTGGTGCCCATCCTGGTCGAGCTGGACGGCGACGTAAACGGCCACA
AGTTCAGCGTGTCCGGCGAGGGCGAGGGCGATGCCACCTACGGCAAG
CTGACCCTGAAGTTCATCTGCACCACCGGCAAGCTGCCCGTGCCCTG
GCCCACCCTCGTGACCACCCTGACCTACGGCGTGCAGTGCTTCAGCC
GCTACCCCGACCACATGAAGCAGCACGACTTCTTCAAGTCCGCCATG
CCCGAAGGCTACGTCCAGGAGCGCACCATCTTCTTCAAGGACGACGG
CAACTACAAGACCCGCGCCGAGGTGAAGTTCGAGGGCGACACCCTGG
TGAACCGCATCGAGCTGAAGGGCATCGACTTCAAGGAGGACGGCAAC
ATCCTGGGGCACAAGCTGGAGTACAACTACAACAGCCACAACGTCTA
TATCATGGCCGACAAGCAGAAGAACGGCATCAAGGTGAACTTCAAGA
TCCGCCACAACATCGAGGACGGCAGCGTGCAGCTCGCCGACCACTAC
CAGCAGAACACCCCCATCGGCGACGGCCCCGTGCTGCTGCCCGACAA
CCACTACCTGAGCACCCAGTCCGCCCTGAGCAAAGACCCCAACGAGA
AGCGCGATCACATGGTCCTGCTGGAGTTCGTGACCGCCGCCGGGATC
ACTCTCGGCATGGACGAGCTGTACAAGTAAcctgcaggcgccggcgt
cctctgagtgctgaaggcttgctgtaggctgtatgctgTTGAGAATC
CTGATGGAGCgggtttttgcctccaactgacccGCTCATAGGATTCT
CAAcagtgtatgatgcctgttactagcattcacatggaGAGCTCGCT
GATCAGCCTCGACTGTGCCTTCTAGTTGCCAGCCATCTGTTGTTTGC
CCCTCCCCCGTGCCTTCCTTGACCCTGGAAGGTGCCACTCCCACTGT
CCTTTCCTAATAAAATGAGGAAATTGCATCGCATTGTCTGAGTAGGT
GTCATTCTATTCTGGGGGGTGGGGTGGGGCAGGACAGCAAGGGGGAG
GATTGGGAAGACAATAGCAGGCATGCTGGGGATGCGGTGGGCTCTAT
GGAAGCTTGAATTCAGCTGACGTGCCTCGGACCGCTAGGAACCCCTA
GTGATGGAGTTGGCCACTCCCTCTCTGCGCGCTCGCTCGCTCACTGA
GGCCGGGCGACCAAAGGTCGCCCGACGCCCGGGCTTTGCCCGGGCGG
CCTCAGTGAGCGAGCGAGCGCGCAG
Exemplary pITR.CAG.miR2-155_EGFP
sequence
(SEQ ID NO: 205)
CTGCGCGCTCGCTCGCTCACTGAGGCCGCCCGGGCGTCGGGCGACCT
TTGGTCGCCCGGCCTCAGTGAGCGAGCGAGCGCGCAGAGAGGGAGTG
GCCAACTCCATCACTAGGGGTTCCTGCGGCCGCACGCGTGACATTGA
TTATTGACTAGTTATTAATAGTAATCAATTACGGGGTCATTAGTTCA
TAGCCCATATATGGAGTTCCGCGTTACATAACTTACGGTAAATGGCC
CGCCTGGCTGACCGCCCAACGACCCCCGCCCATTGACGTCAATAATG
ACGTATGTTCCCATAGTAACGCCAATAGGGACTTTCCATTGACGTCA
ATGGGTGGACTATTTACGGTAAACTGCCCACTTGGCAGTACATCAAG
TGTATCATATGCCAAGTACGCCCCCTATTGACGTCAATGACGGTAAA
TGGCCCGCCTGGCATTATGCCCAGTACATGACCTTATGGGACTTTCC
TACTTGGCAGTACATCTACGTATTAGTCATCGCTATTACCATGGGTC
GAGGTGAGCCCCACGTTCTGCTTCACTCTCCCCATCTCCCCCCCCTC
CCCACCCCCAATTTTGTATTTATTTATTTTTTAATTATTTTGTGCAG
CGATGGGGGCGGGGGGGGGGGGGGCGCGCGCCAGGCGGGGCGGGGCG
GGGCGAGGGGCGGGGCGGGGCGAGGCGGAGAGGTGCGGCGGCAGCCA
ATCAGAGCGGCGCGCTCCGAAAGTTTCCTTTTATGGCGAGGCGGCGG
CGGCGGCGGCCCTATAAAAAGCGAAGCGCGCGGCGGGCGGGAGTCGC
TGCGTTGCCTTCGCCCCGTGCCCCGCTCCGCGCCGCCTCGCGCCGCC
CGCCCCGGCTCTGACTGACCGCGTTACTCCCACAGGTGAGCGGGCGG
GACGGCCCTTCTCCTCCGGGCTGTAATTAGCGCTTGGTTTAATGACG
GCTCGTTTCTTTTCTGTGGCTGCGTGAAAGCCTTAAAGGGCTCCGGG
AGGGCCCTTTGTGCGGGGGGGAGCGGCTCGGGGGGTGCGTGCGTGTG
TGTGTGCGTGGGGAGCGCCGCGTGCGGCCCGCGCTGCCCGGCGGCTG
TGAGCGCTGCGGGCGCGGCGCGGGGCTTTGTGCGCTCCGCGTGTGCG
CGAGGGGAGCGCGGCCGGGGGCGGTGCCCCGCGGTGCGGGGGGGCTG
CGAGGGGAACAAAGGCTGCGTGCGGGGTGTGTGCGTGGGGGGGTGAG
CAGGGGGTGTGGGCGCGGCGGTCGGGCTGTAACCCCCCCCTGCACCC
CCCTCCCCGAGTTGCTGAGCACGGCCCGGCTTCGGGTGCGGGGCTCC
GTGCGGGGCGTGGCGCGGGGCTCGCCGTGCCGGGCGGGGGGTGGCGG
CAGGTGGGGGTGCCGGGCGGGGCGGGGCCGCCTCGGGCCGGGGAGGG
CTCGGGGGAGGGGCGCGGCGGCCCCCGGAGCGCCGGCGtcctctgag
tgctgaaggcttgctgtaggctgtatgctgTTGAGTCTCAGAGATGC
CCgggtttttgcctccaactgaCTCGGGATTCTGAGACTCAAcagtg
tatgatgcctgttactagcattcacatggaTCTAGAGCCTCTGCTAA
CCATGTTCATGCCTTCTTCTTTTTCCTACAGCTCCTGGGCAACGTGC
TGGTTATTGTGACCGGTCGCTAGCCACCATGGTGAGCAAGGGCGAGG
AGCTGTTCACCGGGGTGGTGCCCATCCTGGTCGAGCTGGACGGCGAC
GTAAACGGCCACAAGTTCAGCGTGTCCGGCGAGGGCGAGGGCGATGC
CACCTACGGCAAGCTGACCCTGAAGTTCATCTGCACCACCGGCAAGC
TGCCCGTGCCCTGGCCCACCCTCGTGACCACCCTGACCTACGGCGTG
CAGTGCTTCAGCCGCTACCCCGACCACATGAAGCAGCACGACTTCTT
CAAGTCCGCCATGCCCGAAGGCTACGTCCAGGAGCGCACCATCTTCT
TCAAGGACGACGGCAACTACAAGACCCGCGCCGAGGTGAAGTTCGAG
GGCGACACCCTGGTGAACCGCATCGAGCTGAAGGGCATCGACTTCAA
GGAGGACGGCAACATCCTGGGGCACAAGCTGGAGTACAACTACAACA
GCCACAACGTCTATATCATGGCCGACAAGCAGAAGAACGGCATCAAG
GTGAACTTCAAGATCCGCCACAACATCGAGGACGGCAGCGTGCAGCT
CGCCGACCACTACCAGCAGAACACCCCCATCGGCGACGGCCCCGTGC
TGCTGCCCGACAACCACTACCTGAGCACCCAGTCCGCCCTGAGCAAA
GACCCCAACGAGAAGCGCGATCACATGGTCCTGCTGGAGTTCGTGAC
CGCCGCCGGGATCACTCTCGGCATGGACGAGCTGTACAAGTAAcctg
caggcgccggcgtcctctgagtgctgaaggcttgctgtaggctgtat
gctgTTGAGTCTCAGAGATGCCCgggtttttgcctccaactgaCTCG
GGATTCTGAGACTCAAcagtgtatgatgcctgttactagcattcaca
tggaGAGCTCGCTGATCAGCCTCGACTGTGCCTTCTAGTTGCCAGCC
ATCTGTTGTTTGCCCCTCCCCCGTGCCTTCCTTGACCCTGGAAGGTG
CCACTCCCACTGTCCTTTCCTAATAAAATGAGGAAATTGCATCGCAT
TGTCTGAGTAGGTGTCATTCTATTCTGGGGGGTGGGGTGGGGCAGGA
CAGCAAGGGGGAGGATTGGGAAGACAATAGCAGGCATGCTGGGGATG
CGGTGGGCTCTATGGAAGCTTGAATTCAGCTGACGTGCCTCGGACCG
CTAGGAACCCCTAGTGATGGAGTTGGCCACTCCCTCTCTGCGCGCTC
GCTCGCTCACTGAGGCCGGGCGACCAAAGGTCGCCCGACGCCCGGGC
TTTGCCCGGGCGGCCTCAGTGAGCGAGCGAGCGCGCAG
Exemplary pITR.CAG.miR2v2-155_EGFP
sequence
(SEQ ID NO: 206)
CTGCGCGCTCGCTCGCTCACTGAGGCCGCCCGGGCGTCGGGCGACCT
TTGGTCGCCCGGCCTCAGTGAGCGAGCGAGCGCGCAGAGAGGGAGTG
GCCAACTCCATCACTAGGGGTTCCTGCGGCCGCACGCGTGACATTGA
TTATTGACTAGTTATTAATAGTAATCAATTACGGGGTCATTAGTTCA
TAGCCCATATATGGAGTTCCGCGTTACATAACTTACGGTAAATGGCC
CGCCTGGCTGACCGCCCAACGACCCCCGCCCATTGACGTCAATAATG
ACGTATGTTCCCATAGTAACGCCAATAGGGACTTTCCATTGACGTCA
ATGGGTGGACTATTTACGGTAAACTGCCCACTTGGCAGTACATCAAG
TGTATCATATGCCAAGTACGCCCCCTATTGACGTCAATGACGGTAAA
TGGCCCGCCTGGCATTATGCCCAGTACATGACCTTATGGGACTTTCC
TACTTGGCAGTACATCTACGTATTAGTCATCGCTATTACCATGGGTC
GAGGTGAGCCCCACGTTCTGCTTCACTCTCCCCATCTCCCCCCCCTC
CCCACCCCCAATTTTGTATTTATTTATTTTTTAATTATTTTGTGCAG
CGATGGGGGCGGGGGGGGGGGGGGCGCGCGCCAGGCGGGGCGGGGCG
GGGCGAGGGGCGGGGCGGGGCGAGGCGGAGAGGTGCGGCGGCAGCCA
ATCAGAGCGGCGCGCTCCGAAAGTTTCCTTTTATGGCGAGGCGGCGG
CGGCGGCGGCCCTATAAAAAGCGAAGCGCGCGGCGGGCGGGAGTCGC
TGCGTTGCCTTCGCCCCGTGCCCCGCTCCGCGCCGCCTCGCGCCGCC
CGCCCCGGCTCTGACTGACCGCGTTACTCCCACAGGTGAGCGGGCGG
GACGGCCCTTCTCCTCCGGGCTGTAATTAGCGCTTGGTTTAATGACG
GCTCGTTTCTTTTCTGTGGCTGCGTGAAAGCCTTAAAGGGCTCCGGG
AGGGCCCTTTGTGCGGGGGGGAGCGGCTCGGGGGGTGCGTGCGTGTG
TGTGTGCGTGGGGAGCGCCGCGTGCGGCCCGCGCTGCCCGGCGGCTG
TGAGCGCTGCGGGCGCGGCGCGGGGCTTTGTGCGCTCCGCGTGTGCG
CGAGGGGAGCGCGGCCGGGGGCGGTGCCCCGCGGTGCGGGGGGGCTG
CGAGGGGAACAAAGGCTGCGTGCGGGGTGTGTGCGTGGGGGGGTGAG
CAGGGGGTGTGGGCGCGGCGGTCGGGCTGTAACCCCCCCCTGCACCC
CCCTCCCCGAGTTGCTGAGCACGGCCCGGCTTCGGGTGCGGGGCTCC
GTGCGGGGCGTGGCGCGGGGCTCGCCGTGCCGGGCGGGGGGTGGCGG
CAGGTGGGGGTGCCGGGCGGGGCGGGGCCGCCTCGGGCCGGGGAGGG
CTCGGGGGAGGGGCGCGGCGGCCCCCGGAGCGCCGGCGtcctctgag
tgctgaaggcttgctgtaggctgtatgctgTTGAGTCTCAGAGATGC
CCgggtttttgcctccaactgaCTCGGGATTCTGAGATTCAAcagtg
tatgatgcctgttactagcattcacatggaTCTAGAGCCTCTGCTAA
CCATGTTCATGCCTTCTTCTTTTTCCTACAGCTCCTGGGCAACGTGC
TGGTTATTGTGACCGGTCGCTAGCCACCATGGTGAGCAAGGGCGAGG
AGCTGTTCACCGGGGTGGTGCCCATCCTGGTCGAGCTGGACGGCGAC
GTAAACGGCCACAAGTTCAGCGTGTCCGGCGAGGGCGAGGGCGATGC
CACCTACGGCAAGCTGACCCTGAAGTTCATCTGCACCACCGGCAAGC
TGCCCGTGCCCTGGCCCACCCTCGTGACCACCCTGACCTACGGCGTG
CAGTGCTTCAGCCGCTACCCCGACCACATGAAGCAGCACGACTTCTT
CAAGTCCGCCATGCCCGAAGGCTACGTCCAGGAGCGCACCATCTTCT
TCAAGGACGACGGCAACTACAAGACCCGCGCCGAGGTGAAGTTCGAG
GGCGACACCCTGGTGAACCGCATCGAGCTGAAGGGCATCGACTTCAA
GGAGGACGGCAACATCCTGGGGCACAAGCTGGAGTACAACTACAACA
GCCACAACGTCTATATCATGGCCGACAAGCAGAAGAACGGCATCAAG
GTGAACTTCAAGATCCGCCACAACATCGAGGACGGCAGCGTGCAGCT
CGCCGACCACTACCAGCAGAACACCCCCATCGGCGACGGCCCCGTGC
TGCTGCCCGACAACCACTACCTGAGCACCCAGTCCGCCCTGAGCAAA
GACCCCAACGAGAAGCGCGATCACATGGTCCTGCTGGAGTTCGTGAC
CGCCGCCGGGATCACTCTCGGCATGGACGAGCTGTACAAGTAAcctg
caggcgccggcgtcctctgagtgctgaaggcttgctgtaggctgtat
gctgTTGAGTCTCAGAGATGCCCgggtttttgcctccaactgaCTCG
GGATTCTGAGATTCAAcagtgtatgatgcctgttactagcattcaca
tggaGAGCTCGCTGATCAGCCTCGACTGTGCCTTCTAGTTGCCAGCC
ATCTGTTGTTTGCCCCTCCCCCGTGCCTTCCTTGACCCTGGAAGGTG
CCACTCCCACTGTCCTTTCCTAATAAAATGAGGAAATTGCATCGCAT
TGTCTGAGTAGGTGTCATTCTATTCTGGGGGGTGGGGTGGGGCAGGA
CAGCAAGGGGGAGGATTGGGAAGACAATAGCAGGCATGCTGGGGATG
CGGTGGGCTCTATGGAAGCTTGAATTCAGCTGACGTGCCTCGGACCG
CTAGGAACCCCTAGTGATGGAGTTGGCCACTCCCTCTCTGCGCGCTC
GCTCGCTCACTGAGGCCGGGCGACCAAAGGTCGCCCGACGCCCGGGC
TTTGCCCGGGCGGCCTCAGTGAGCGAGCGAGCGCGCAG
Exemplary pITR.CAG.miR2v3-155_EGFP
sequence
(SEQ ID NO: 207)
CTGCGCGCTCGCTCGCTCACTGAGGCCGCCCGGGCGTCGGGCGACCT
TTGGTCGCCCGGCCTCAGTGAGCGAGCGAGCGCGCAGAGAGGGAGTG
GCCAACTCCATCACTAGGGGTTCCTGCGGCCGCACGCGTGACATTGA
TTATTGACTAGTTATTAATAGTAATCAATTACGGGGTCATTAGTTCA
TAGCCCATATATGGAGTTCCGCGTTACATAACTTACGGTAAATGGCC
CGCCTGGCTGACCGCCCAACGACCCCCGCCCATTGACGTCAATAATG
ACGTATGTTCCCATAGTAACGCCAATAGGGACTTTCCATTGACGTCA
ATGGGTGGACTATTTACGGTAAACTGCCCACTTGGCAGTACATCAAG
TGTATCATATGCCAAGTACGCCCCCTATTGACGTCAATGACGGTAAA
TGGCCCGCCTGGCATTATGCCCAGTACATGACCTTATGGGACTTTCC
TACTTGGCAGTACATCTACGTATTAGTCATCGCTATTACCATGGGTC
GAGGTGAGCCCCACGTTCTGCTTCACTCTCCCCATCTCCCCCCCCTC
CCCACCCCCAATTTTGTATTTATTTATTTTTTAATTATTTTGTGCAG
CGATGGGGGCGGGGGGGGGGGGGGCGCGCGCCAGGCGGGGCGGGGCG
GGGCGAGGGGCGGGGCGGGGCGAGGCGGAGAGGTGCGGCGGCAGCCA
ATCAGAGCGGCGCGCTCCGAAAGTTTCCTTTTATGGCGAGGCGGCGG
CGGCGGCGGCCCTATAAAAAGCGAAGCGCGCGGCGGGCGGGAGTCGC
TGCGTTGCCTTCGCCCCGTGCCCCGCTCCGCGCCGCCTCGCGCCGCC
CGCCCCGGCTCTGACTGACCGCGTTACTCCCACAGGTGAGCGGGCGG
GACGGCCCTTCTCCTCCGGGCTGTAATTAGCGCTTGGTTTAATGACG
GCTCGTTTCTTTTCTGTGGCTGCGTGAAAGCCTTAAAGGGCTCCGGG
AGGGCCCTTTGTGCGGGGGGGAGCGGCTCGGGGGGTGCGTGCGTGTG
TGTGTGCGTGGGGAGCGCCGCGTGCGGCCCGCGCTGCCCGGCGGCTG
TGAGCGCTGCGGGCGCGGCGCGGGGCTTTGTGCGCTCCGCGTGTGCG
CGAGGGGAGCGCGGCCGGGGGCGGTGCCCCGCGGTGCGGGGGGGCTG
CGAGGGGAACAAAGGCTGCGTGCGGGGTGTGTGCGTGGGGGGGTGAG
CAGGGGGTGTGGGCGCGGCGGTCGGGCTGTAACCCCCCCCTGCACCC
CCCTCCCCGAGTTGCTGAGCACGGCCCGGCTTCGGGTGCGGGGCTCC
GTGCGGGGCGTGGCGCGGGGCTCGCCGTGCCGGGCGGGGGGTGGCGG
CAGGTGGGGGTGCCGGGCGGGGCGGGGCCGCCTCGGGCCGGGGAGGG
CTCGGGGGAGGGGCGCGGCGGCCCCCGGAGCGCCGGCGtcctctgag
tgctgaaggcttgctgtaggctgtatgctgTTGAGTCTCAGAGATGC
CCgggtttttgcctccaactgaCTCGGGATCCTGAGATTCAAcagtg
tatgatgcctgttactagcattcacatggaTCTAGAGCCTCTGCTAA
CCATGTTCATGCCTTCTTCTTTTTCCTACAGCTCCTGGGCAACGTGC
TGGTTATTGTGACCGGTCGCTAGCCACCATGGTGAGCAAGGGCGAGG
AGCTGTTCACCGGGGTGGTGCCCATCCTGGTCGAGCTGGACGGCGAC
GTAAACGGCCACAAGTTCAGCGTGTCCGGCGAGGGCGAGGGCGATGC
CACCTACGGCAAGCTGACCCTGAAGTTCATCTGCACCACCGGCAAGC
TGCCCGTGCCCTGGCCCACCCTCGTGACCACCCTGACCTACGGCGTG
CAGTGCTTCAGCCGCTACCCCGACCACATGAAGCAGCACGACTTCTT
CAAGTCCGCCATGCCCGAAGGCTACGTCCAGGAGCGCACCATCTTCT
TCAAGGACGACGGCAACTACAAGACCCGCGCCGAGGTGAAGTTCGAG
GGCGACACCCTGGTGAACCGCATCGAGCTGAAGGGCATCGACTTCAA
GGAGGACGGCAACATCCTGGGGCACAAGCTGGAGTACAACTACAACA
GCCACAACGTCTATATCATGGCCGACAAGCAGAAGAACGGCATCAAG
GTGAACTTCAAGATCCGCCACAACATCGAGGACGGCAGCGTGCAGCT
CGCCGACCACTACCAGCAGAACACCCCCATCGGCGACGGCCCCGTGC
TGCTGCCCGACAACCACTACCTGAGCACCCAGTCCGCCCTGAGCAAA
GACCCCAACGAGAAGCGCGATCACATGGTCCTGCTGGAGTTCGTGAC
CGCCGCCGGGATCACTCTCGGCATGGACGAGCTGTACAAGTAAcctg
caggcgccggcgtcctctgagtgctgaaggcttgctgtaggctgtat
gctgTTGAGTCTCAGAGATGCCCgggtttttgcctccaactgaCTCG
GGATCCTGAGATTCAAcagtgtatgatgcctgttactagcattcaca
tggaGAGCTCGCTGATCAGCCTCGACTGTGCCTTCTAGTTGCCAGCC
ATCTGTTGTTTGCCCCTCCCCCGTGCCTTCCTTGACCCTGGAAGGTG
CCACTCCCACTGTCCTTTCCTAATAAAATGAGGAAATTGCATCGCAT
TGTCTGAGTAGGTGTCATTCTATTCTGGGGGGTGGGGTGGGGCAGGA
CAGCAAGGGGGAGGATTGGGAAGACAATAGCAGGCATGCTGGGGATG
CGGTGGGCTCTATGGAAGCTTGAATTCAGCTGACGTGCCTCGGACCG
CTAGGAACCCCTAGTGATGGAGTTGGCCACTCCCTCTCTGCGCGCTC
GCTCGCTCACTGAGGCCGGGCGACCAAAGGTCGCCCGACGCCCGGGC
TTTGCCCGGGCGGCCTCAGTGAGCGAGCGAGCGCGCAG
Exemplary pITR.CAG.miR2v4-155_EGFP
sequence
(SEQ ID NO: 208)
CTGCGCGCTCGCTCGCTCACTGAGGCCGCCCGGGCGTCGGGCGACCT
TTGGTCGCCCGGCCTCAGTGAGCGAGCGAGCGCGCAGAGAGGGAGTG
GCCAACTCCATCACTAGGGGTTCCTGCGGCCGCACGCGTGACATTGA
TTATTGACTAGTTATTAATAGTAATCAATTACGGGGTCATTAGTTCA
TAGCCCATATATGGAGTTCCGCGTTACATAACTTACGGTAAATGGCC
CGCCTGGCTGACCGCCCAACGACCCCCGCCCATTGACGTCAATAATG
ACGTATGTTCCCATAGTAACGCCAATAGGGACTTTCCATTGACGTCA
ATGGGTGGACTATTTACGGTAAACTGCCCACTTGGCAGTACATCAAG
TGTATCATATGCCAAGTACGCCCCCTATTGACGTCAATGACGGTAAA
TGGCCCGCCTGGCATTATGCCCAGTACATGACCTTATGGGACTTTCC
TACTTGGCAGTACATCTACGTATTAGTCATCGCTATTACCATGGGTC
GAGGTGAGCCCCACGTTCTGCTTCACTCTCCCCATCTCCCCCCCCTC
CCCACCCCCAATTTTGTATTTATTTATTTTTTAATTATTTTGTGCAG
CGATGGGGGCGGGGGGGGGGGGGGCGCGCGCCAGGCGGGGCGGGGCG
GGGCGAGGGGCGGGGCGGGGCGAGGCGGAGAGGTGCGGCGGCAGCCA
ATCAGAGCGGCGCGCTCCGAAAGTTTCCTTTTATGGCGAGGCGGCGG
CGGCGGCGGCCCTATAAAAAGCGAAGCGCGCGGCGGGCGGGAGTCGC
TGCGTTGCCTTCGCCCCGTGCCCCGCTCCGCGCCGCCTCGCGCCGCC
CGCCCCGGCTCTGACTGACCGCGTTACTCCCACAGGTGAGCGGGCGG
GACGGCCCTTCTCCTCCGGGCTGTAATTAGCGCTTGGTTTAATGACG
GCTCGTTTCTTTTCTGTGGCTGCGTGAAAGCCTTAAAGGGCTCCGGG
AGGGCCCTTTGTGCGGGGGGGAGCGGCTCGGGGGGTGCGTGCGTGTG
TGTGTGCGTGGGGAGCGCCGCGTGCGGCCCGCGCTGCCCGGCGGCTG
TGAGCGCTGCGGGCGCGGCGCGGGGCTTTGTGCGCTCCGCGTGTGCG
CGAGGGGAGCGCGGCCGGGGGCGGTGCCCCGCGGTGCGGGGGGGCTG
CGAGGGGAACAAAGGCTGCGTGCGGGGTGTGTGCGTGGGGGGGTGAG
CAGGGGGTGTGGGCGCGGCGGTCGGGCTGTAACCCCCCCCTGCACCC
CCCTCCCCGAGTTGCTGAGCACGGCCCGGCTTCGGGTGCGGGGCTCC
GTGCGGGGCGTGGCGCGGGGCTCGCCGTGCCGGGCGGGGGGTGGCGG
CAGGTGGGGGTGCCGGGCGGGGCGGGGCCGCCTCGGGCCGGGGAGGG
CTCGGGGGAGGGGCGCGGCGGCCCCCGGAGCGCCGGCGtcctctgag
tgctgaaggcttgctgtaggctgtatgctgTTGAGTCTCAGAGATGC
CCggaagtttgcctccaactctTTCGGGATCCTGAGATTCAAcagtg
tatgatgcctgttactagcattcacatggaTCTAGAGCCTCTGCTAA
CCATGTTCATGCCTTCTTCTTTTTCCTACAGCTCCTGGGCAACGTGC
TGGTTATTGTGACCGGTCGCTAGCCACCATGGTGAGCAAGGGCGAGG
AGCTGTTCACCGGGGTGGTGCCCATCCTGGTCGAGCTGGACGGCGAC
GTAAACGGCCACAAGTTCAGCGTGTCCGGCGAGGGCGAGGGCGATGC
CACCTACGGCAAGCTGACCCTGAAGTTCATCTGCACCACCGGCAAGC
TGCCCGTGCCCTGGCCCACCCTCGTGACCACCCTGACCTACGGCGTG
CAGTGCTTCAGCCGCTACCCCGACCACATGAAGCAGCACGACTTCTT
CAAGTCCGCCATGCCCGAAGGCTACGTCCAGGAGCGCACCATCTTCT
TCAAGGACGACGGCAACTACAAGACCCGCGCCGAGGTGAAGTTCGAG
GGCGACACCCTGGTGAACCGCATCGAGCTGAAGGGCATCGACTTCAA
GGAGGACGGCAACATCCTGGGGCACAAGCTGGAGTACAACTACAACA
GCCACAACGTCTATATCATGGCCGACAAGCAGAAGAACGGCATCAAG
GTGAACTTCAAGATCCGCCACAACATCGAGGACGGCAGCGTGCAGCT
CGCCGACCACTACCAGCAGAACACCCCCATCGGCGACGGCCCCGTGC
TGCTGCCCGACAACCACTACCTGAGCACCCAGTCCGCCCTGAGCAAA
GACCCCAACGAGAAGCGCGATCACATGGTCCTGCTGGAGTTCGTGAC
CGCCGCCGGGATCACTCTCGGCATGGACGAGCTGTACAAGTAAcctg
caggcgccggcgtcctctgagtgctgaaggcttgctgtaggctgtat
gctgTTGAGTCTCAGAGATGCCCggaagtttgcctccaactctTTCG
GGATCCTGAGATTCAAcagtgtatgatgcctgttactagcattcaca
tggaGAGCTCGCTGATCAGCCTCGACTGTGCCTTCTAGTTGCCAGCC
ATCTGTTGTTTGCCCCTCCCCCGTGCCTTCCTTGACCCTGGAAGGTG
CCACTCCCACTGTCCTTTCCTAATAAAATGAGGAAATTGCATCGCAT
TGTCTGAGTAGGTGTCATTCTATTCTGGGGGGTGGGGTGGGGCAGGA
CAGCAAGGGGGAGGATTGGGAAGACAATAGCAGGCATGCTGGGGATG
CGGTGGGCTCTATGGAAGCTTGAATTCAGCTGACGTGCCTCGGACCG
CTAGGAACCCCTAGTGATGGAGTTGGCCACTCCCTCTCTGCGCGCTC
GCTCGCTCACTGAGGCCGGGCGACCAAAGGTCGCCCGACGCCCGGGC
TTTGCCCGGGCGGCCTCAGTGAGCGAGCGAGCGCGCAG
Exemplary pITR.CAG.miR4-155_EGFP
sequence
(SEQ ID NO: 209)
CTGCGCGCTCGCTCGCTCACTGAGGCCGCCCGGGCGTCGGGCGACCT
TTGGTCGCCCGGCCTCAGTGAGCGAGCGAGCGCGCAGAGAGGGAGTG
GCCAACTCCATCACTAGGGGTTCCTGCGGCCGCACGCGTGACATTGA
TTATTGACTAGTTATTAATAGTAATCAATTACGGGGTCATTAGTTCA
TAGCCCATATATGGAGTTCCGCGTTACATAACTTACGGTAAATGGCC
CGCCTGGCTGACCGCCCAACGACCCCCGCCCATTGACGTCAATAATG
ACGTATGTTCCCATAGTAACGCCAATAGGGACTTTCCATTGACGTCA
ATGGGTGGACTATTTACGGTAAACTGCCCACTTGGCAGTACATCAAG
TGTATCATATGCCAAGTACGCCCCCTATTGACGTCAATGACGGTAAA
TGGCCCGCCTGGCATTATGCCCAGTACATGACCTTATGGGACTTTCC
TACTTGGCAGTACATCTACGTATTAGTCATCGCTATTACCATGGGTC
GAGGTGAGCCCCACGTTCTGCTTCACTCTCCCCATCTCCCCCCCCTC
CCCACCCCCAATTTTGTATTTATTTATTTTTTAATTATTTTGTGCAG
CGATGGGGGCGGGGGGGGGGGGGGCGCGCGCCAGGCGGGGCGGGGCG
GGGCGAGGGGCGGGGCGGGGCGAGGCGGAGAGGTGCGGCGGCAGCCA
ATCAGAGCGGCGCGCTCCGAAAGTTTCCTTTTATGGCGAGGCGGCGG
CGGCGGCGGCCCTATAAAAAGCGAAGCGCGCGGCGGGCGGGAGTCGC
TGCGTTGCCTTCGCCCCGTGCCCCGCTCCGCGCCGCCTCGCGCCGCC
CGCCCCGGCTCTGACTGACCGCGTTACTCCCACAGGTGAGCGGGCGG
GACGGCCCTTCTCCTCCGGGCTGTAATTAGCGCTTGGTTTAATGACG
GCTCGTTTCTTTTCTGTGGCTGCGTGAAAGCCTTAAAGGGCTCCGGG
AGGGCCCTTTGTGCGGGGGGGAGCGGCTCGGGGGGTGCGTGCGTGTG
TGTGTGCGTGGGGAGCGCCGCGTGCGGCCCGCGCTGCCCGGCGGCTG
TGAGCGCTGCGGGCGCGGCGCGGGGCTTTGTGCGCTCCGCGTGTGCG
CGAGGGGAGCGCGGCCGGGGGCGGTGCCCCGCGGTGCGGGGGGGCTG
CGAGGGGAACAAAGGCTGCGTGCGGGGTGTGTGCGTGGGGGGGTGAG
CAGGGGGTGTGGGCGCGGCGGTCGGGCTGTAACCCCCCCCTGCACCC
CCCTCCCCGAGTTGCTGAGCACGGCCCGGCTTCGGGTGCGGGGCTCC
GTGCGGGGCGTGGCGCGGGGCTCGCCGTGCCGGGCGGGGGGTGGCGG
CAGGTGGGGGTGCCGGGCGGGGCGGGGCCGCCTCGGGCCGGGGAGGG
CTCGGGGGAGGGGCGCGGCGGCCCCCGGAGCGCCGGCGtcctctgag
tgctgaaggcttgctgtaggctgtatgctgTTCTCGAAGTGCTTCTG
CCgggtttttgcctccaactgacTcGGCGAATACTTCGAGAAcagtg
tatgatgcctgttactagcattcacatggaTCTAGAGCCTCTGCTAA
CCATGTTCATGCCTTCTTCTTTTTCCTACAGCTCCTGGGCAACGTGC
TGGTTATTGTGACCGGTCGCTAGCCACCATGGTGAGCAAGGGCGAGG
AGCTGTTCACCGGGGTGGTGCCCATCCTGGTCGAGCTGGACGGCGAC
GTAAACGGCCACAAGTTCAGCGTGTCCGGCGAGGGCGAGGGCGATGC
CACCTACGGCAAGCTGACCCTGAAGTTCATCTGCACCACCGGCAAGC
TGCCCGTGCCCTGGCCCACCCTCGTGACCACCCTGACCTACGGCGTG
CAGTGCTTCAGCCGCTACCCCGACCACATGAAGCAGCACGACTTCTT
CAAGTCCGCCATGCCCGAAGGCTACGTCCAGGAGCGCACCATCTTCT
TCAAGGACGACGGCAACTACAAGACCCGCGCCGAGGTGAAGTTCGAG
GGCGACACCCTGGTGAACCGCATCGAGCTGAAGGGCATCGACTTCAA
GGAGGACGGCAACATCCTGGGGCACAAGCTGGAGTACAACTACAACA
GCCACAACGTCTATATCATGGCCGACAAGCAGAAGAACGGCATCAAG
GTGAACTTCAAGATCCGCCACAACATCGAGGACGGCAGCGTGCAGCT
CGCCGACCACTACCAGCAGAACACCCCCATCGGCGACGGCCCCGTGC
TGCTGCCCGACAACCACTACCTGAGCACCCAGTCCGCCCTGAGCAAA
GACCCCAACGAGAAGCGCGATCACATGGTCCTGCTGGAGTTCGTGAC
CGCCGCCGGGATCACTCTCGGCATGGACGAGCTGTACAAGTAAcctg
caggcgccggcgtcctctgagtgctgaaggcttgctgtaggctgtat
gctgTTCTCGAAGTGCTTCTGCCgggtttttgcctccaactgacTcG
GCGAATACTTCGAGAAcagtgtatgatgcctgttactagcattcaca
tggaGAGCTCGCTGATCAGCCTCGACTGTGCCTTCTAGTTGCCAGCC
ATCTGTTGTTTGCCCCTCCCCCGTGCCTTCCTTGACCCTGGAAGGTG
CCACTCCCACTGTCCTTTCCTAATAAAATGAGGAAATTGCATCGCAT
TGTCTGAGTAGGTGTCATTCTATTCTGGGGGGTGGGGTGGGGCAGGA
CAGCAAGGGGGAGGATTGGGAAGACAATAGCAGGCATGCTGGGGATG
CGGTGGGCTCTATGGAAGCTTGAATTCAGCTGACGTGCCTCGGACCG
CTAGGAACCCCTAGTGATGGAGTTGGCCACTCCCTCTCTGCGCGCTC
GCTCGCTCACTGAGGCCGGGCGACCAAAGGTCGCCCGACGCCCGGGC
TTTGCCCGGGCGGCCTCAGTGAGCGAGCGAGCGCGCAG
Exemplary pITR.CAG.miR5-155_EGFP
sequence
(SEQ ID NO: 210)
CTGCGCGCTCGCTCGCTCACTGAGGCCGCCCGGGCGTCGGGCGACCT
TTGGTCGCCCGGCCTCAGTGAGCGAGCGAGCGCGCAGAGAGGGAGTG
GCCAACTCCATCACTAGGGGTTCCTGCGGCCGCACGCGTGACATTGA
TTATTGACTAGTTATTAATAGTAATCAATTACGGGGTCATTAGTTCA
TAGCCCATATATGGAGTTCCGCGTTACATAACTTACGGTAAATGGCC
CGCCTGGCTGACCGCCCAACGACCCCCGCCCATTGACGTCAATAATG
ACGTATGTTCCCATAGTAACGCCAATAGGGACTTTCCATTGACGTCA
ATGGGTGGACTATTTACGGTAAACTGCCCACTTGGCAGTACATCAAG
TGTATCATATGCCAAGTACGCCCCCTATTGACGTCAATGACGGTAAA
TGGCCCGCCTGGCATTATGCCCAGTACATGACCTTATGGGACTTTCC
TACTTGGCAGTACATCTACGTATTAGTCATCGCTATTACCATGGGTC
GAGGTGAGCCCCACGTTCTGCTTCACTCTCCCCATCTCCCCCCCCTC
CCCACCCCCAATTTTGTATTTATTTATTTTTTAATTATTTTGTGCAG
CGATGGGGGCGGGGGGGGGGGGGGCGCGCGCCAGGCGGGGCGGGGCG
GGGCGAGGGGCGGGGCGGGGCGAGGCGGAGAGGTGCGGCGGCAGCCA
ATCAGAGCGGCGCGCTCCGAAAGTTTCCTTTTATGGCGAGGCGGCGG
CGGCGGCGGCCCTATAAAAAGCGAAGCGCGCGGCGGGCGGGAGTCGC
TGCGTTGCCTTCGCCCCGTGCCCCGCTCCGCGCCGCCTCGCGCCGCC
CGCCCCGGCTCTGACTGACCGCGTTACTCCCACAGGTGAGCGGGCGG
GACGGCCCTTCTCCTCCGGGCTGTAATTAGCGCTTGGTTTAATGACG
GCTCGTTTCTTTTCTGTGGCTGCGTGAAAGCCTTAAAGGGCTCCGGG
AGGGCCCTTTGTGCGGGGGGGAGCGGCTCGGGGGGTGCGTGCGTGTG
TGTGTGCGTGGGGAGCGCCGCGTGCGGCCCGCGCTGCCCGGCGGCTG
TGAGCGCTGCGGGCGCGGCGCGGGGCTTTGTGCGCTCCGCGTGTGCG
CGAGGGGAGCGCGGCCGGGGGCGGTGCCCCGCGGTGCGGGGGGGCTG
CGAGGGGAACAAAGGCTGCGTGCGGGGTGTGTGCGTGGGGGGGTGAG
CAGGGGGTGTGGGCGCGGCGGTCGGGCTGTAACCCCCCCCTGCACCC
CCCTCCCCGAGTTGCTGAGCACGGCCCGGCTTCGGGTGCGGGGCTCC
GTGCGGGGCGTGGCGCGGGGCTCGCCGTGCCGGGCGGGGGGTGGCGG
CAGGTGGGGGTGCCGGGCGGGGCGGGGCCGCCTCGGGCCGGGGAGGG
CTCGGGGGAGGGGCGCGGCGGCCCCCGGAGCGCCGGCGtcctctgag
tgctgaaggcttgctgtaggctgtatgctgTTCTCCACCTTGACCAC
GCgtgtttttgcctccaactgacaTGTGGGTAAGGTGGAGAACAgtg
tatgatgcctgttactagcattcacatggaTCTAGAGCCTCTGCTAA
CCATGTTCATGCCTTCTTCTTTTTCCTACAGCTCCTGGGCAACGTGC
TGGTTATTGTGACCGGTCGCTAGCCACCATGGTGAGCAAGGGCGAGG
AGCTGTTCACCGGGGTGGTGCCCATCCTGGTCGAGCTGGACGGCGAC
GTAAACGGCCACAAGTTCAGCGTGTCCGGCGAGGGCGAGGGCGATGC
CACCTACGGCAAGCTGACCCTGAAGTTCATCTGCACCACCGGCAAGC
TGCCCGTGCCCTGGCCCACCCTCGTGACCACCCTGACCTACGGCGTG
CAGTGCTTCAGCCGCTACCCCGACCACATGAAGCAGCACGACTTCTT
CAAGTCCGCCATGCCCGAAGGCTACGTCCAGGAGCGCACCATCTTCT
TCAAGGACGACGGCAACTACAAGACCCGCGCCGAGGTGAAGTTCGAG
GGCGACACCCTGGTGAACCGCATCGAGCTGAAGGGCATCGACTTCAA
GGAGGACGGCAACATCCTGGGGCACAAGCTGGAGTACAACTACAACA
GCCACAACGTCTATATCATGGCCGACAAGCAGAAGAACGGCATCAAG
GTGAACTTCAAGATCCGCCACAACATCGAGGACGGCAGCGTGCAGCT
CGCCGACCACTACCAGCAGAACACCCCCATCGGCGACGGCCCCGTGC
TGCTGCCCGACAACCACTACCTGAGCACCCAGTCCGCCCTGAGCAAA
GACCCCAACGAGAAGCGCGATCACATGGTCCTGCTGGAGTTCGTGAC
CGCCGCCGGGATCACTCTCGGCATGGACGAGCTGTACAAGTAAcctg
caggcgccggcgtcctctgagtgctgaaggcttgctgtaggctgtat
gctgTTCTCCACCTTGACCACGCgtgtttttgcctccaactgacaTG
TGGGTAAGGTGGAGAACAgtgtatgatgcctgttactagcattcaca
tggaGAGCTCGCTGATCAGCCTCGACTGTGCCTTCTAGTTGCCAGCC
ATCTGTTGTTTGCCCCTCCCCCGTGCCTTCCTTGACCCTGGAAGGTG
CCACTCCCACTGTCCTTTCCTAATAAAATGAGGAAATTGCATCGCAT
TGTCTGAGTAGGTGTCATTCTATTCTGGGGGGTGGGGTGGGGCAGGA
CAGCAAGGGGGAGGATTGGGAAGACAATAGCAGGCATGCTGGGGATG
CGGTGGGCTCTATGGAAGCTTGAATTCAGCTGACGTGCCTCGGACCG
CTAGGAACCCCTAGTGATGGAGTTGGCCACTCCCTCTCTGCGCGCTC
GCTCGCTCACTGAGGCCGGGCGACCAAAGGTCGCCCGACGCCCGGGC
TTTGCCCGGGCGGCCTCAGTGAGCGAGCGAGCGCGCAG
Exemplary pITR.CAG.miR6-155_EGFP
sequence
(SEQ ID NO: 211)
CTGCGCGCTCGCTCGCTCACTGAGGCCGCCCGGGCGTCGGGCGACCT
TTGGTCGCCCGGCCTCAGTGAGCGAGCGAGCGCGCAGAGAGGGAGTG
GCCAACTCCATCACTAGGGGTTCCTGCGGCCGCACGCGTGACATTGA
TTATTGACTAGTTATTAATAGTAATCAATTACGGGGTCATTAGTTCA
TAGCCCATATATGGAGTTCCGCGTTACATAACTTACGGTAAATGGCC
CGCCTGGCTGACCGCCCAACGACCCCCGCCCATTGACGTCAATAATG
ACGTATGTTCCCATAGTAACGCCAATAGGGACTTTCCATTGACGTCA
ATGGGTGGACTATTTACGGTAAACTGCCCACTTGGCAGTACATCAAG
TGTATCATATGCCAAGTACGCCCCCTATTGACGTCAATGACGGTAAA
TGGCCCGCCTGGCATTATGCCCAGTACATGACCTTATGGGACTTTCC
TACTTGGCAGTACATCTACGTATTAGTCATCGCTATTACCATGGGTC
GAGGTGAGCCCCACGTTCTGCTTCACTCTCCCCATCTCCCCCCCCTC
CCCACCCCCAATTTTGTATTTATTTATTTTTTAATTATTTTGTGCAG
CGATGGGGGCGGGGGGGGGGGGGGCGCGCGCCAGGCGGGGCGGGGCG
GGGCGAGGGGCGGGGCGGGGCGAGGCGGAGAGGTGCGGCGGCAGCCA
ATCAGAGCGGCGCGCTCCGAAAGTTTCCTTTTATGGCGAGGCGGCGG
CGGCGGCGGCCCTATAAAAAGCGAAGCGCGCGGCGGGCGGGAGTCGC
TGCGTTGCCTTCGCCCCGTGCCCCGCTCCGCGCCGCCTCGCGCCGCC
CGCCCCGGCTCTGACTGACCGCGTTACTCCCACAGGTGAGCGGGCGG
GACGGCCCTTCTCCTCCGGGCTGTAATTAGCGCTTGGTTTAATGACG
GCTCGTTTCTTTTCTGTGGCTGCGTGAAAGCCTTAAAGGGCTCCGGG
AGGGCCCTTTGTGCGGGGGGGAGCGGCTCGGGGGGTGCGTGCGTGTG
TGTGTGCGTGGGGAGCGCCGCGTGCGGCCCGCGCTGCCCGGCGGCTG
TGAGCGCTGCGGGCGCGGCGCGGGGCTTTGTGCGCTCCGCGTGTGCG
CGAGGGGAGCGCGGCCGGGGGCGGTGCCCCGCGGTGCGGGGGGGCTG
CGAGGGGAACAAAGGCTGCGTGCGGGGTGTGTGCGTGGGGGGGTGAG
CAGGGGGTGTGGGCGCGGCGGTCGGGCTGTAACCCCCCCCTGCACCC
CCCTCCCCGAGTTGCTGAGCACGGCCCGGCTTCGGGTGCGGGGCTCC
GTGCGGGGCGTGGCGCGGGGCTCGCCGTGCCGGGCGGGGGGTGGCGG
CAGGTGGGGGTGCCGGGCGGGGCGGGGCCGCCTCGGGCCGGGGAGGG
CTCGGGGGAGGGGCGCGGCGGCCCCCGGAGCGCCGGCGtcctctgag
tgctgaaggcttgctgtaggctgtatgctgATACTGTCATAGTAGTA
CCaggtttttgcctccaactgacctGGTCTATATGACAGTATCAgtg
tatgatgcctgttactagcattcacatggaTCTAGAGCCTCTGCTAA
CCATGTTCATGCCTTCTTCTTTTTCCTACAGCTCCTGGGCAACGTGC
TGGTTATTGTGACCGGTCGCTAGCCACCATGGTGAGCAAGGGCGAGG
AGCTGTTCACCGGGGTGGTGCCCATCCTGGTCGAGCTGGACGGCGAC
GTAAACGGCCACAAGTTCAGCGTGTCCGGCGAGGGCGAGGGCGATGC
CACCTACGGCAAGCTGACCCTGAAGTTCATCTGCACCACCGGCAAGC
TGCCCGTGCCCTGGCCCACCCTCGTGACCACCCTGACCTACGGCGTG
CAGTGCTTCAGCCGCTACCCCGACCACATGAAGCAGCACGACTTCTT
CAAGTCCGCCATGCCCGAAGGCTACGTCCAGGAGCGCACCATCTTCT
TCAAGGACGACGGCAACTACAAGACCCGCGCCGAGGTGAAGTTCGAG
GGCGACACCCTGGTGAACCGCATCGAGCTGAAGGGCATCGACTTCAA
GGAGGACGGCAACATCCTGGGGCACAAGCTGGAGTACAACTACAACA
GCCACAACGTCTATATCATGGCCGACAAGCAGAAGAACGGCATCAAG
GTGAACTTCAAGATCCGCCACAACATCGAGGACGGCAGCGTGCAGCT
CGCCGACCACTACCAGCAGAACACCCCCATCGGCGACGGCCCCGTGC
TGCTGCCCGACAACCACTACCTGAGCACCCAGTCCGCCCTGAGCAAA
GACCCCAACGAGAAGCGCGATCACATGGTCCTGCTGGAGTTCGTGAC
CGCCGCCGGGATCACTCTCGGCATGGACGAGCTGTACAAGTAAcctg
caggcgccggcgtcctctgagtgctgaaggcttgctgtaggctgtat
gctgATACTGTCATAGTAGTACCaggtttttgcctccaactgacctG
GTCTATATGACAGTATCAgtgtatgatgcctgttactagcattcaca
tggaGAGCTCGCTGATCAGCCTCGACTGTGCCTTCTAGTTGCCAGCC
ATCTGTTGTTTGCCCCTCCCCCGTGCCTTCCTTGACCCTGGAAGGTG
CCACTCCCACTGTCCTTTCCTAATAAAATGAGGAAATTGCATCGCAT
TGTCTGAGTAGGTGTCATTCTATTCTGGGGGGTGGGGTGGGGCAGGA
CAGCAAGGGGGAGGATTGGGAAGACAATAGCAGGCATGCTGGGGATG
CGGTGGGCTCTATGGAAGCTTGAATTCAGCTGACGTGCCTCGGACCG
CTAGGAACCCCTAGTGATGGAGTTGGCCACTCCCTCTCTGCGCGCTC
GCTCGCTCACTGAGGCCGGGCGACCAAAGGTCGCCCGACGCCCGGGC
TTTGCCCGGGCGGCCTCAGTGAGCGAGCGAGCGCGCAG
Exemplary pITR.CAG.miR7-155_EGFP
sequence
(SEQ ID NO: 212)
CTGCGCGCTCGCTCGCTCACTGAGGCCGCCCGGGCGTCGGGCGACCT
TTGGTCGCCCGGCCTCAGTGAGCGAGCGAGCGCGCAGAGAGGGAGTG
GCCAACTCCATCACTAGGGGTTCCTGCGGCCGCACGCGTGACATTGA
TTATTGACTAGTTATTAATAGTAATCAATTACGGGGTCATTAGTTCA
TAGCCCATATATGGAGTTCCGCGTTACATAACTTACGGTAAATGGCC
CGCCTGGCTGACCGCCCAACGACCCCCGCCCATTGACGTCAATAATG
ACGTATGTTCCCATAGTAACGCCAATAGGGACTTTCCATTGACGTCA
ATGGGTGGACTATTTACGGTAAACTGCCCACTTGGCAGTACATCAAG
TGTATCATATGCCAAGTACGCCCCCTATTGACGTCAATGACGGTAAA
TGGCCCGCCTGGCATTATGCCCAGTACATGACCTTATGGGACTTTCC
TACTTGGCAGTACATCTACGTATTAGTCATCGCTATTACCATGGGTC
GAGGTGAGCCCCACGTTCTGCTTCACTCTCCCCATCTCCCCCCCCTC
CCCACCCCCAATTTTGTATTTATTTATTTTTTAATTATTTTGTGCAG
CGATGGGGGCGGGGGGGGGGGGGGCGCGCGCCAGGCGGGGCGGGGCG
GGGCGAGGGGCGGGGCGGGGCGAGGCGGAGAGGTGCGGCGGCAGCCA
ATCAGAGCGGCGCGCTCCGAAAGTTTCCTTTTATGGCGAGGCGGCGG
CGGCGGCGGCCCTATAAAAAGCGAAGCGCGCGGCGGGCGGGAGTCGC
TGCGTTGCCTTCGCCCCGTGCCCCGCTCCGCGCCGCCTCGCGCCGCC
CGCCCCGGCTCTGACTGACCGCGTTACTCCCACAGGTGAGCGGGCGG
GACGGCCCTTCTCCTCCGGGCTGTAATTAGCGCTTGGTTTAATGACG
GCTCGTTTCTTTTCTGTGGCTGCGTGAAAGCCTTAAAGGGCTCCGGG
AGGGCCCTTTGTGCGGGGGGGAGCGGCTCGGGGGGTGCGTGCGTGTG
TGTGTGCGTGGGGAGCGCCGCGTGCGGCCCGCGCTGCCCGGCGGCTG
TGAGCGCTGCGGGCGCGGCGCGGGGCTTTGTGCGCTCCGCGTGTGCG
CGAGGGGAGCGCGGCCGGGGGCGGTGCCCCGCGGTGCGGGGGGGCTG
CGAGGGGAACAAAGGCTGCGTGCGGGGTGTGTGCGTGGGGGGGTGAG
CAGGGGGTGTGGGCGCGGCGGTCGGGCTGTAACCCCCCCCTGCACCC
CCCTCCCCGAGTTGCTGAGCACGGCCCGGCTTCGGGTGCGGGGCTCC
GTGCGGGGCGTGGCGCGGGGCTCGCCGTGCCGGGCGGGGGGTGGCGG
CAGGTGGGGGTGCCGGGCGGGGCGGGGCCGCCTCGGGCCGGGGAGGG
CTCGGGGGAGGGGCGCGGCGGCCCCCGGAGCGCCGGCGTCCTCTGAG
TGCTGAAGGCTTGCTGTAGGCTGTATGCTGTAGTCGGAGGTGATGTC
GGGGGTTTTTGCCTCCAACTGACTCCTGCATACCTTCGATTACAGTG
TATGATGCCTGTTACTAGCATTCACATGGATCTAGAGCCTCTGCTAA
CCATGTTCATGCCTTCTTCTTTTTCCTACAGCTCCTGGGCAACGTGC
TGGTTATTGTGACCGGTCGCTAGCCACCATGGTGAGCAAGGGCGAGG
AGCTGTTCACCGGGGTGGTGCCCATCCTGGTCGAGCTGGACGGCGAC
GTAAACGGCCACAAGTTCAGCGTGTCCGGCGAGGGCGAGGGCGATGC
CACCTACGGCAAGCTGACCCTGAAGTTCATCTGCACCACCGGCAAGC
TGCCCGTGCCCTGGCCCACCCTCGTGACCACCCTGACCTACGGCGTG
CAGTGCTTCAGCCGCTACCCCGACCACATGAAGCAGCACGACTTCTT
CAAGTCCGCCATGCCCGAAGGCTACGTCCAGGAGCGCACCATCTTCT
TCAAGGACGACGGCAACTACAAGACCCGCGCCGAGGTGAAGTTCGAG
GGCGACACCCTGGTGAACCGCATCGAGCTGAAGGGCATCGACTTCAA
GGAGGACGGCAACATCCTGGGGCACAAGCTGGAGTACAACTACAACA
GCCACAACGTCTATATCATGGCCGACAAGCAGAAGAACGGCATCAAG
GTGAACTTCAAGATCCGCCACAACATCGAGGACGGCAGCGTGCAGCT
CGCCGACCACTACCAGCAGAACACCCCCATCGGCGACGGCCCCGTGC
TGCTGCCCGACAACCACTACCTGAGCACCCAGTCCGCCCTGAGCAAA
GACCCCAACGAGAAGCGCGATCACATGGTCCTGCTGGAGTTCGTGAC
CGCCGCCGGGATCACTCTCGGCATGGACGAGCTGTACAAGTAACCTG
CAGGCGCCGGCGTCCTCTGAGTGCTGAAGGCTTGCTGTAGGCTGTAT
GCTGTAGTCGGAGGTGATGTCGGGGGTTTTTGCCTCCAACTGACTCC
TGCATACCTTCGATTACAGTGTATGATGCCTGTTACTAGCATTCACA
TGGAGAGCTCGCTGATCAGCCTCGACTGTGCCTTCTAGTTGCCAGCC
ATCTGTTGTTTGCCCCTCCCCCGTGCCTTCCTTGACCCTGGAAGGTG
CCACTCCCACTGTCCTTTCCTAATAAAATGAGGAAATTGCATCGCAT
TGTCTGAGTAGGTGTCATTCTATTCTGGGGGGTGGGGTGGGGCAGGA
CAGCAAGGGGGAGGATTGGGAAGACAATAGCAGGCATGCTGGGGATG
CGGTGGGCTCTATGGAAGCTTGAATTCAGCTGACGTGCCTCGGACCG
CTAGGAACCCCTAGTGATGGAGTTGGCCACTCCCTCTCTGCGCGCTC
GCTCGCTCACTGAGGCCGGGCGACCAAAGGTCGCCCGACGCCCGGGC
TTTGCCCGGGCGGCCTCAGTGAGCGAGCGAGCGCGCAG
Exemplary pITR.CAG.miR1-16_EGFP
sequence
(SEQ ID NO: 213)
CTGCGCGCTCGCTCGCTCACTGAGGCCGCCCGGGCGTCGGGCGACCT
TTGGTCGCCCGGCCTCAGTGAGCGAGCGAGCGCGCAGAGAGGGAGTG
GCCAACTCCATCACTAGGGGTTCCTGCGGCCGCACGCGTGACATTGA
TTATTGACTAGTTATTAATAGTAATCAATTACGGGGTCATTAGTTCA
TAGCCCATATATGGAGTTCCGCGTTACATAACTTACGGTAAATGGCC
CGCCTGGCTGACCGCCCAACGACCCCCGCCCATTGACGTCAATAATG
ACGTATGTTCCCATAGTAACGCCAATAGGGACTTTCCATTGACGTCA
ATGGGTGGACTATTTACGGTAAACTGCCCACTTGGCAGTACATCAAG
TGTATCATATGCCAAGTACGCCCCCTATTGACGTCAATGACGGTAAA
TGGCCCGCCTGGCATTATGCCCAGTACATGACCTTATGGGACTTTCC
TACTTGGCAGTACATCTACGTATTAGTCATCGCTATTACCATGGGTC
GAGGTGAGCCCCACGTTCTGCTTCACTCTCCCCATCTCCCCCCCCTC
CCCACCCCCAATTTTGTATTTATTTATTTTTTAATTATTTTGTGCAG
CGATGGGGGCGGGGGGGGGGGGGGCGCGCGCCAGGCGGGGCGGGGCG
GGGCGAGGGGCGGGGCGGGGCGAGGCGGAGAGGTGCGGCGGCAGCCA
ATCAGAGCGGCGCGCTCCGAAAGTTTCCTTTTATGGCGAGGCGGCGG
CGGCGGCGGCCCTATAAAAAGCGAAGCGCGCGGCGGGCGGGAGTCGC
TGCGTTGCCTTCGCCCCGTGCCCCGCTCCGCGCCGCCTCGCGCCGCC
CGCCCCGGCTCTGACTGACCGCGTTACTCCCACAGGTGAGCGGGCGG
GACGGCCCTTCTCCTCCGGGCTGTAATTAGCGCTTGGTTTAATGACG
GCTCGTTTCTTTTCTGTGGCTGCGTGAAAGCCTTAAAGGGCTCCGGG
AGGGCCCTTTGTGCGGGGGGGAGCGGCTCGGGGGGTGCGTGCGTGTG
TGTGTGCGTGGGGAGCGCCGCGTGCGGCCCGCGCTGCCCGGCGGCTG
TGAGCGCTGCGGGCGCGGCGCGGGGCTTTGTGCGCTCCGCGTGTGCG
CGAGGGGAGCGCGGCCGGGGGCGGTGCCCCGCGGTGCGGGGGGGCTG
CGAGGGGAACAAAGGCTGCGTGCGGGGTGTGTGCGTGGGGGGGTGAG
CAGGGGGTGTGGGCGCGGCGGTCGGGCTGTAACCCCCCCCTGCACCC
CCCTCCCCGAGTTGCTGAGCACGGCCCGGCTTCGGGTGCGGGGCTCC
GTGCGGGGCGTGGCGCGGGGCTCGCCGTGCCGGGCGGGGGGTGGCGG
CAGGTGGGGGTGCCGGGCGGGGCGGGGCCGCCTCGGGCCGGGGAGGG
CTCGGGGGAGGGGCGCGGCGGCCCCCGGAGCGCCGGCGCATAGCTCT
TATGATAGCAATGTCAGCAGTGCCTTTGAGAATCCTGATGGAGCggg
TTAAGATTCTAAAATTATCAcGCTCCATGAGTGATTCTCAGAAGTAA
GGTTGACCATACTCTACAGTTGTGTCTAGAGCCTCTGCTAACCATGT
TCATGCCTTCTTCTTTTTCCTACAGCTCCTGGGCAACGTGCTGGTTA
TTGTGACCGGTCGCTAGCCACCATGGTGAGCAAGGGCGAGGAGCTGT
TCACCGGGGTGGTGCCCATCCTGGTCGAGCTGGACGGCGACGTAAAC
GGCCACAAGTTCAGCGTGTCCGGCGAGGGCGAGGGCGATGCCACCTA
CGGCAAGCTGACCCTGAAGTTCATCTGCACCACCGGCAAGCTGCCCG
TGCCCTGGCCCACCCTCGTGACCACCCTGACCTACGGCGTGCAGTGC
TTCAGCCGCTACCCCGACCACATGAAGCAGCACGACTTCTTCAAGTC
CGCCATGCCCGAAGGCTACGTCCAGGAGCGCACCATCTTCTTCAAGG
ACGACGGCAACTACAAGACCCGCGCCGAGGTGAAGTTCGAGGGCGAC
ACCCTGGTGAACCGCATCGAGCTGAAGGGCATCGACTTCAAGGAGGA
CGGCAACATCCTGGGGCACAAGCTGGAGTACAACTACAACAGCCACA
ACGTCTATATCATGGCCGACAAGCAGAAGAACGGCATCAAGGTGAAC
TTCAAGATCCGCCACAACATCGAGGACGGCAGCGTGCAGCTCGCCGA
CCACTACCAGCAGAACACCCCCATCGGCGACGGCCCCGTGCTGCTGC
CCGACAACCACTACCTGAGCACCCAGTCCGCCCTGAGCAAAGACCCC
AACGAGAAGCGCGATCACATGGTCCTGCTGGAGTTCGTGACCGCCGC
CGGGATCACTCTCGGCATGGACGAGCTGTACAAGTAAcctgcaggcg
ccggcgCATAGCTCTTATGATAGCAATGTCAGCAGTGCCTTTGAGAA
TCCTGATGGAGCgggTTAAGATTCTAAAATTATCAcGCTCCATGAGT
GATTCTCAGAAGTAAGGTTGACCATACTCTACAGTTGTGGAGCTCGC
TGATCAGCCTCGACTGTGCCTTCTAGTTGCCAGCCATCTGTTGTTTG
CCCCTCCCCCGTGCCTTCCTTGACCCTGGAAGGTGCCACTCCCACTG
TCCTTTCCTAATAAAATGAGGAAATTGCATCGCATTGTCTGAGTAGG
TGTCATTCTATTCTGGGGGGTGGGGTGGGGCAGGACAGCAAGGGGGA
GGATTGGGAAGACAATAGCAGGCATGCTGGGGATGCGGTGGGCTCTA
TGGAAGCTTGAATTCAGCTGACGTGCCTCGGACCGCTAGGAACCCCT
AGTGATGGAGTTGGCCACTCCCTCTCTGCGCGCTCGCTCGCTCACTG
AGGCCGGGCGACCAAAGGTCGCCCGACGCCCGGGCTTTGCCCGGGCG
GCCTCAGTGAGCGAGCGAGCGCGCAG
Exemplary pITR.CAG.miR1-26_EGFP
sequence
(SEQ ID NO: 214)
CTGCGCGCTCGCTCGCTCACTGAGGCCGCCCGGGCGTCGGGCGACCT
TTGGTCGCCCGGCCTCAGTGAGCGAGCGAGCGCGCAGAGAGGGAGTG
GCCAACTCCATCACTAGGGGTTCCTGCGGCCGCACGCGTGACATTGA
TTATTGACTAGTTATTAATAGTAATCAATTACGGGGTCATTAGTTCA
TAGCCCATATATGGAGTTCCGCGTTACATAACTTACGGTAAATGGCC
CGCCTGGCTGACCGCCCAACGACCCCCGCCCATTGACGTCAATAATG
ACGTATGTTCCCATAGTAACGCCAATAGGGACTTTCCATTGACGTCA
ATGGGTGGACTATTTACGGTAAACTGCCCACTTGGCAGTACATCAAG
TGTATCATATGCCAAGTACGCCCCCTATTGACGTCAATGACGGTAAA
TGGCCCGCCTGGCATTATGCCCAGTACATGACCTTATGGGACTTTCC
TACTTGGCAGTACATCTACGTATTAGTCATCGCTATTACCATGGGTC
GAGGTGAGCCCCACGTTCTGCTTCACTCTCCCCATCTCCCCCCCCTC
CCCACCCCCAATTTTGTATTTATTTATTTTTTAATTATTTTGTGCAG
CGATGGGGGCGGGGGGGGGGGGGGCGCGCGCCAGGCGGGGCGGGGCG
GGGCGAGGGGCGGGGCGGGGCGAGGCGGAGAGGTGCGGCGGCAGCCA
ATCAGAGCGGCGCGCTCCGAAAGTTTCCTTTTATGGCGAGGCGGCGG
CGGCGGCGGCCCTATAAAAAGCGAAGCGCGCGGCGGGCGGGAGTCGC
TGCGTTGCCTTCGCCCCGTGCCCCGCTCCGCGCCGCCTCGCGCCGCC
CGCCCCGGCTCTGACTGACCGCGTTACTCCCACAGGTGAGCGGGCGG
GACGGCCCTTCTCCTCCGGGCTGTAATTAGCGCTTGGTTTAATGACG
GCTCGTTTCTTTTCTGTGGCTGCGTGAAAGCCTTAAAGGGCTCCGGG
AGGGCCCTTTGTGCGGGGGGGAGCGGCTCGGGGGGTGCGTGCGTGTG
TGTGTGCGTGGGGAGCGCCGCGTGCGGCCCGCGCTGCCCGGCGGCTG
TGAGCGCTGCGGGCGCGGCGCGGGGCTTTGTGCGCTCCGCGTGTGCG
CGAGGGGAGCGCGGCCGGGGGCGGTGCCCCGCGGTGCGGGGGGGCTG
CGAGGGGAACAAAGGCTGCGTGCGGGGTGTGTGCGTGGGGGGGTGAG
CAGGGGGTGTGGGCGCGGCGGTCGGGCTGTAACCCCCCCCTGCACCC
CCCTCCCCGAGTTGCTGAGCACGGCCCGGCTTCGGGTGCGGGGCTCC
GTGCGGGGCGTGGCGCGGGGCTCGCCGTGCCGGGCGGGGGGTGGCGG
CAGGTGGGGGTGCCGGGCGGGGCGGGGCCGCCTCGGGCCGGGGAGGG
CTCGGGGGAGGGGCGCGGCGGCCCCCGGAGCGCCGGCGGAGCAGGAG
GACCAAGGCCCTGGCGAAGGCCGTGGCCTCGTTGAGAATCCTGATGG
AGCggTGTGCAGGTCCCAATGGccGCTCCATAAGGATTCTCCACGGG
GACGCGGGCCTGGACGCCGGCATCCGGGCTCAGGACCCCCCTCTCTA
GAGCCTCTGCTAACCATGTTCATGCCTTCTTCTTTTTCCTACAGCTC
CTGGGCAACGTGCTGGTTATTGTGACCGGTCGCTAGCCACCATGGTG
AGCAAGGGCGAGGAGCTGTTCACCGGGGTGGTGCCCATCCTGGTCGA
GCTGGACGGCGACGTAAACGGCCACAAGTTCAGCGTGTCCGGCGAGG
GCGAGGGCGATGCCACCTACGGCAAGCTGACCCTGAAGTTCATCTGC
ACCACCGGCAAGCTGCCCGTGCCCTGGCCCACCCTCGTGACCACCCT
GACCTACGGCGTGCAGTGCTTCAGCCGCTACCCCGACCACATGAAGC
AGCACGACTTCTTCAAGTCCGCCATGCCCGAAGGCTACGTCCAGGAG
CGCACCATCTTCTTCAAGGACGACGGCAACTACAAGACCCGCGCCGA
GGTGAAGTTCGAGGGCGACACCCTGGTGAACCGCATCGAGCTGAAGG
GCATCGACTTCAAGGAGGACGGCAACATCCTGGGGCACAAGCTGGAG
TACAACTACAACAGCCACAACGTCTATATCATGGCCGACAAGCAGAA
GAACGGCATCAAGGTGAACTTCAAGATCCGCCACAACATCGAGGACG
GCAGCGTGCAGCTCGCCGACCACTACCAGCAGAACACCCCCATCGGC
GACGGCCCCGTGCTGCTGCCCGACAACCACTACCTGAGCACCCAGTC
CGCCCTGAGCAAAGACCCCAACGAGAAGCGCGATCACATGGTCCTGC
TGGAGTTCGTGACCGCCGCCGGGATCACTCTCGGCATGGACGAGCTG
TACAAGTAAcctgcaggcgccggcgGAGCAGGAGGACCAAGGCCCTG
GCGAAGGCCGTGGCCTCGTTGAGAATCCTGATGGAGCggTGTGCAGG
TCCCAATGGccGCTCCATAAGGATTCTCCACGGGGACGCGGGCCTGG
ACGCCGGCATCCGGGCTCAGGACCCCCCTCGAGCTCGCTGATCAGCC
TCGACTGTGCCTTCTAGTTGCCAGCCATCTGTTGTTTGCCCCTCCCC
CGTGCCTTCCTTGACCCTGGAAGGTGCCACTCCCACTGTCCTTTCCT
AATAAAATGAGGAAATTGCATCGCATTGTCTGAGTAGGTGTCATTCT
ATTCTGGGGGGTGGGGTGGGGCAGGACAGCAAGGGGGAGGATTGGGA
AGACAATAGCAGGCATGCTGGGGATGCGGTGGGCTCTATGGAAGCTT
GAATTCAGCTGACGTGCCTCGGACCGCTAGGAACCCCTAGTGATGGA
GTTGGCCACTCCCTCTCTGCGCGCTCGCTCGCTCACTGAGGCCGGGC
GACCAAAGGTCGCCCGACGCCCGGGCTTTGCCCGGGCGGCCTCAGTG
AGCGAGCGAGCGCGCAG
Exemplary pITR.CAG.miR1-96_EGFP
sequence
(SEQ ID NO: 215)
CTGCGCGCTCGCTCGCTCACTGAGGCCGCCCGGGCGTCGGGCGACCT
TTGGTCGCCCGGCCTCAGTGAGCGAGCGAGCGCGCAGAGAGGGAGTG
GCCAACTCCATCACTAGGGGTTCCTGCGGCCGCACGCGTGACATTGA
TTATTGACTAGTTATTAATAGTAATCAATTACGGGGTCATTAGTTCA
TAGCCCATATATGGAGTTCCGCGTTACATAACTTACGGTAAATGGCC
CGCCTGGCTGACCGCCCAACGACCCCCGCCCATTGACGTCAATAATG
ACGTATGTTCCCATAGTAACGCCAATAGGGACTTTCCATTGACGTCA
ATGGGTGGACTATTTACGGTAAACTGCCCACTTGGCAGTACATCAAG
TGTATCATATGCCAAGTACGCCCCCTATTGACGTCAATGACGGTAAA
TGGCCCGCCTGGCATTATGCCCAGTACATGACCTTATGGGACTTTCC
TACTTGGCAGTACATCTACGTATTAGTCATCGCTATTACCATGGGTC
GAGGTGAGCCCCACGTTCTGCTTCACTCTCCCCATCTCCCCCCCCTC
CCCACCCCCAATTTTGTATTTATTTATTTTTTAATTATTTTGTGCAG
CGATGGGGGCGGGGGGGGGGGGGGCGCGCGCCAGGCGGGGCGGGGCG
GGGCGAGGGGCGGGGCGGGGCGAGGCGGAGAGGTGCGGCGGCAGCCA
ATCAGAGCGGCGCGCTCCGAAAGTTTCCTTTTATGGCGAGGCGGCGG
CGGCGGCGGCCCTATAAAAAGCGAAGCGCGCGGCGGGCGGGAGTCGC
TGCGTTGCCTTCGCCCCGTGCCCCGCTCCGCGCCGCCTCGCGCCGCC
CGCCCCGGCTCTGACTGACCGCGTTACTCCCACAGGTGAGCGGGCGG
GACGGCCCTTCTCCTCCGGGCTGTAATTAGCGCTTGGTTTAATGACG
GCTCGTTTCTTTTCTGTGGCTGCGTGAAAGCCTTAAAGGGCTCCGGG
AGGGCCCTTTGTGCGGGGGGGAGCGGCTCGGGGGGTGCGTGCGTGTG
TGTGTGCGTGGGGAGCGCCGCGTGCGGCCCGCGCTGCCCGGCGGCTG
TGAGCGCTGCGGGCGCGGCGCGGGGCTTTGTGCGCTCCGCGTGTGCG
CGAGGGGAGCGCGGCCGGGGGCGGTGCCCCGCGGTGCGGGGGGGCTG
CGAGGGGAACAAAGGCTGCGTGCGGGGTGTGTGCGTGGGGGGGTGAG
CAGGGGGTGTGGGCGCGGCGGTCGGGCTGTAACCCCCCCCTGCACCC
CCCTCCCCGAGTTGCTGAGCACGGCCCGGCTTCGGGTGCGGGGCTCC
GTGCGGGGCGTGGCGCGGGGCTCGCCGTGCCGGGCGGGGGGTGGCGG
CAGGTGGGGGTGCCGGGCGGGGCGGGGCCGCCTCGGGCCGGGGAGGG
CTCGGGGGAGGGGCGCGGCGGCCCCCGGAGCGCCGGCGGTGTCCCCA
GAGAGCCCGCACCAGTGCCATCTGCTTGGCCGATTTGAGAATCCTGA
TGGAGCggCTTGTGTCTCTCCGCTCTGAGccGGGCCATCAGATTCTC
ATATGGGAAAAGCAGGACCCGCAGCTGCGTCCGCCTCCCCTGCATTC
TAGAGCCTCTGCTAACCATGTTCATGCCTTCTTCTTTTTCCTACAGC
TCCTGGGCAACGTGCTGGTTATTGTGACCGGTCGCTAGCCACCATGG
TGAGCAAGGGCGAGGAGCTGTTCACCGGGGTGGTGCCCATCCTGGTC
GAGCTGGACGGCGACGTAAACGGCCACAAGTTCAGCGTGTCCGGCGA
GGGCGAGGGCGATGCCACCTACGGCAAGCTGACCCTGAAGTTCATCT
GCACCACCGGCAAGCTGCCCGTGCCCTGGCCCACCCTCGTGACCACC
CTGACCTACGGCGTGCAGTGCTTCAGCCGCTACCCCGACCACATGAA
GCAGCACGACTTCTTCAAGTCCGCCATGCCCGAAGGCTACGTCCAGG
AGCGCACCATCTTCTTCAAGGACGACGGCAACTACAAGACCCGCGCC
GAGGTGAAGTTCGAGGGCGACACCCTGGTGAACCGCATCGAGCTGAA
GGGCATCGACTTCAAGGAGGACGGCAACATCCTGGGGCACAAGCTGG
AGTACAACTACAACAGCCACAACGTCTATATCATGGCCGACAAGCAG
AAGAACGGCATCAAGGTGAACTTCAAGATCCGCCACAACATCGAGGA
CGGCAGCGTGCAGCTCGCCGACCACTACCAGCAGAACACCCCCATCG
GCGACGGCCCCGTGCTGCTGCCCGACAACCACTACCTGAGCACCCAG
TCCGCCCTGAGCAAAGACCCCAACGAGAAGCGCGATCACATGGTCCT
GCTGGAGTTCGTGACCGCCGCCGGGATCACTCTCGGCATGGACGAGC
TGTACAAGTAAcctgcaggcgccggcgGTGTCCCCAGAGAGCCCGCA
CCAGTGCCATCTGCTTGGCCGATTTGAGAATCCTGATGGAGCggCTT
GTGTCTCTCCGCTCTGAGccGGGCCATCAGATTCTCATATGGGAAAA
GCAGGACCCGCAGCTGCGTCCGCCTCCCCTGCATGAGCTCGCTGATC
AGCCTCGACTGTGCCTTCTAGTTGCCAGCCATCTGTTGTTTGCCCCT
CCCCCGTGCCTTCCTTGACCCTGGAAGGTGCCACTCCCACTGTCCTT
TCCTAATAAAATGAGGAAATTGCATCGCATTGTCTGAGTAGGTGTCA
TTCTATTCTGGGGGGTGGGGTGGGGCAGGACAGCAAGGGGGAGGATT
GGGAAGACAATAGCAGGCATGCTGGGGATGCGGTGGGCTCTATGGAA
GCTTGAATTCAGCTGACGTGCCTCGGACCGCTAGGAACCCCTAGTGA
TGGAGTTGGCCACTCCCTCTCTGCGCGCTCGCTCGCTCACTGAGGCC
GGGCGACCAAAGGTCGCCCGACGCCCGGGCTTTGCCCGGGCGGCCTC
AGTGAGCGAGCGAGCGCGCAG
Exemplary pITR.CAG.miR1-122_EGFP
sequence
(SEQ ID NO: 216)
CTGCGCGCTCGCTCGCTCACTGAGGCCGCCCGGGCGTCGGGCGACCT
TTGGTCGCCCGGCCTCAGTGAGCGAGCGAGCGCGCAGAGAGGGAGTG
GCCAACTCCATCACTAGGGGTTCCTGCGGCCGCACGCGTGACATTGA
TTATTGACTAGTTATTAATAGTAATCAATTACGGGGTCATTAGTTCA
TAGCCCATATATGGAGTTCCGCGTTACATAACTTACGGTAAATGGCC
CGCCTGGCTGACCGCCCAACGACCCCCGCCCATTGACGTCAATAATG
ACGTATGTTCCCATAGTAACGCCAATAGGGACTTTCCATTGACGTCA
ATGGGTGGACTATTTACGGTAAACTGCCCACTTGGCAGTACATCAAG
TGTATCATATGCCAAGTACGCCCCCTATTGACGTCAATGACGGTAAA
TGGCCCGCCTGGCATTATGCCCAGTACATGACCTTATGGGACTTTCC
TACTTGGCAGTACATCTACGTATTAGTCATCGCTATTACCATGGGTC
GAGGTGAGCCCCACGTTCTGCTTCACTCTCCCCATCTCCCCCCCCTC
CCCACCCCCAATTTTGTATTTATTTATTTTTTAATTATTTTGTGCAG
CGATGGGGGCGGGGGGGGGGGGGGCGCGCGCCAGGCGGGGCGGGGCG
GGGCGAGGGGCGGGGCGGGGCGAGGCGGAGAGGTGCGGCGGCAGCCA
ATCAGAGCGGCGCGCTCCGAAAGTTTCCTTTTATGGCGAGGCGGCGG
CGGCGGCGGCCCTATAAAAAGCGAAGCGCGCGGCGGGCGGGAGTCGC
TGCGTTGCCTTCGCCCCGTGCCCCGCTCCGCGCCGCCTCGCGCCGCC
CGCCCCGGCTCTGACTGACCGCGTTACTCCCACAGGTGAGCGGGCGG
GACGGCCCTTCTCCTCCGGGCTGTAATTAGCGCTTGGTTTAATGACG
GCTCGTTTCTTTTCTGTGGCTGCGTGAAAGCCTTAAAGGGCTCCGGG
AGGGCCCTTTGTGCGGGGGGGAGCGGCTCGGGGGGTGCGTGCGTGTG
TGTGTGCGTGGGGAGCGCCGCGTGCGGCCCGCGCTGCCCGGCGGCTG
TGAGCGCTGCGGGCGCGGCGCGGGGCTTTGTGCGCTCCGCGTGTGCG
CGAGGGGAGCGCGGCCGGGGGCGGTGCCCCGCGGTGCGGGGGGGCTG
CGAGGGGAACAAAGGCTGCGTGCGGGGTGTGTGCGTGGGGGGGTGAG
CAGGGGGTGTGGGCGCGGCGGTCGGGCTGTAACCCCCCCCTGCACCC
CCCTCCCCGAGTTGCTGAGCACGGCCCGGCTTCGGGTGCGGGGCTCC
GTGCGGGGCGTGGCGCGGGGCTCGCCGTGCCGGGCGGGGGGTGGCGG
CAGGTGGGGGTGCCGGGCGGGGCGGGGCCGCCTCGGGCCGGGGAGGG
CTCGGGGGAGGGGCGCGGCGGCCCCCGGAGCGCCGGCGTGAAGTTAA
CACCTTCGTGGCTACAGAGTTTCCTTAGCAGAGCTGTTGAGAATCCT
GATGGAGCggGTGTCTAAACTATCccGCTCCATCcGGATTCTACATA
GCTACTGCTAGGCAATCCTTCCCTCGATAAATGTCTTGGCATCGTCT
AGAGCCTCTGCTAACCATGTTCATGCCTTCTTCTTTTTCCTACAGCT
CCTGGGCAACGTGCTGGTTATTGTGACCGGTCGCTAGCCACCATGGT
GAGCAAGGGCGAGGAGCTGTTCACCGGGGTGGTGCCCATCCTGGTCG
AGCTGGACGGCGACGTAAACGGCCACAAGTTCAGCGTGTCCGGCGAG
GGCGAGGGCGATGCCACCTACGGCAAGCTGACCCTGAAGTTCATCTG
CACCACCGGCAAGCTGCCCGTGCCCTGGCCCACCCTCGTGACCACCC
TGACCTACGGCGTGCAGTGCTTCAGCCGCTACCCCGACCACATGAAG
CAGCACGACTTCTTCAAGTCCGCCATGCCCGAAGGCTACGTCCAGGA
GCGCACCATCTTCTTCAAGGACGACGGCAACTACAAGACCCGCGCCG
AGGTGAAGTTCGAGGGCGACACCCTGGTGAACCGCATCGAGCTGAAG
GGCATCGACTTCAAGGAGGACGGCAACATCCTGGGGCACAAGCTGGA
GTACAACTACAACAGCCACAACGTCTATATCATGGCCGACAAGCAGA
AGAACGGCATCAAGGTGAACTTCAAGATCCGCCACAACATCGAGGAC
GGCAGCGTGCAGCTCGCCGACCACTACCAGCAGAACACCCCCATCGG
CGACGGCCCCGTGCTGCTGCCCGACAACCACTACCTGAGCACCCAGT
CCGCCCTGAGCAAAGACCCCAACGAGAAGCGCGATCACATGGTCCTG
CTGGAGTTCGTGACCGCCGCCGGGATCACTCTCGGCATGGACGAGCT
GTACAAGTAAcctgcaggcgccggcgTGAAGTTAACACCTTCGTGGC
TACAGAGTTTCCTTAGCAGAGCTGTTGAGAATCCTGATGGAGCggGT
GTCTAAACTATCccGCTCCATCcGGATTCTACATAGCTACTGCTAGG
CAATCCTTCCCTCGATAAATGTCTTGGCATCGGAGCTCGCTGATCAG
CCTCGACTGTGCCTTCTAGTTGCCAGCCATCTGTTGTTTGCCCCTCC
CCCGTGCCTTCCTTGACCCTGGAAGGTGCCACTCCCACTGTCCTTTC
CTAATAAAATGAGGAAATTGCATCGCATTGTCTGAGTAGGTGTCATT
CTATTCTGGGGGGTGGGGTGGGGCAGGACAGCAAGGGGGAGGATTGG
GAAGACAATAGCAGGCATGCTGGGGATGCGGTGGGCTCTATGGAAGC
TTGAATTCAGCTGACGTGCCTCGGACCGCTAGGAACCCCTAGTGATG
GAGTTGGCCACTCCCTCTCTGCGCGCTCGCTCGCTCACTGAGGCCGG
GCGACCAAAGGTCGCCCGACGCCCGGGCTTTGCCCGGGCGGCCTCAG
TGAGCGAGCGAGCGCGCAG
Exemplary pITR.CAG.miR1-135_EGFP
sequence
(SEQ ID NO: 217)
CTGCGCGCTCGCTCGCTCACTGAGGCCGCCCGGGCGTCGGGCGACCT
TTGGTCGCCCGGCCTCAGTGAGCGAGCGAGCGCGCAGAGAGGGAGTG
GCCAACTCCATCACTAGGGGTTCCTGCGGCCGCACGCGTGACATTGA
TTATTGACTAGTTATTAATAGTAATCAATTACGGGGTCATTAGTTCA
TAGCCCATATATGGAGTTCCGCGTTACATAACTTACGGTAAATGGCC
CGCCTGGCTGACCGCCCAACGACCCCCGCCCATTGACGTCAATAATG
ACGTATGTTCCCATAGTAACGCCAATAGGGACTTTCCATTGACGTCA
ATGGGTGGACTATTTACGGTAAACTGCCCACTTGGCAGTACATCAAG
TGTATCATATGCCAAGTACGCCCCCTATTGACGTCAATGACGGTAAA
TGGCCCGCCTGGCATTATGCCCAGTACATGACCTTATGGGACTTTCC
TACTTGGCAGTACATCTACGTATTAGTCATCGCTATTACCATGGGTC
GAGGTGAGCCCCACGTTCTGCTTCACTCTCCCCATCTCCCCCCCCTC
CCCACCCCCAATTTTGTATTTATTTATTTTTTAATTATTTTGTGCAG
CGATGGGGGCGGGGGGGGGGGGGGCGCGCGCCAGGCGGGGCGGGGCG
GGGCGAGGGGCGGGGCGGGGCGAGGCGGAGAGGTGCGGCGGCAGCCA
ATCAGAGCGGCGCGCTCCGAAAGTTTCCTTTTATGGCGAGGCGGCGG
CGGCGGCGGCCCTATAAAAAGCGAAGCGCGCGGCGGGCGGGAGTCGC
TGCGTTGCCTTCGCCCCGTGCCCCGCTCCGCGCCGCCTCGCGCCGCC
CGCCCCGGCTCTGACTGACCGCGTTACTCCCACAGGTGAGCGGGCGG
GACGGCCCTTCTCCTCCGGGCTGTAATTAGCGCTTGGTTTAATGACG
GCTCGTTTCTTTTCTGTGGCTGCGTGAAAGCCTTAAAGGGCTCCGGG
AGGGCCCTTTGTGCGGGGGGGAGCGGCTCGGGGGGTGCGTGCGTGTG
TGTGTGCGTGGGGAGCGCCGCGTGCGGCCCGCGCTGCCCGGCGGCTG
TGAGCGCTGCGGGCGCGGCGCGGGGCTTTGTGCGCTCCGCGTGTGCG
CGAGGGGAGCGCGGCCGGGGGCGGTGCCCCGCGGTGCGGGGGGGCTG
CGAGGGGAACAAAGGCTGCGTGCGGGGTGTGTGCGTGGGGGGGTGAG
CAGGGGGTGTGGGCGCGGCGGTCGGGCTGTAACCCCCCCCTGCACCC
CCCTCCCCGAGTTGCTGAGCACGGCCCGGCTTCGGGTGCGGGGCTCC
GTGCGGGGCGTGGCGCGGGGCTCGCCGTGCCGGGCGGGGGGTGGCGG
CAGGTGGGGGTGCCGGGCGGGGCGGGGCCGCCTCGGGCCGGGGAGGG
CTCGGGGGAGGGGCGCGGCGGCCCCCGGAGCGCCGGCGGCCGCTGGA
CCCCTCCACTCTGCTGTGGCCTTGAGAATCCTGATGGAGCggGATTG
CTGTCCCAAACTCccGCTCCATCGGATTCTCAAGGCTACAGTGAGGG
GCGAGCTCCTTCTCCTGCTCTAGAGCCTCTGCTAACCATGTTCATGC
CTTCTTCTTTTTCCTACAGCTCCTGGGCAACGTGCTGGTTATTGTGA
CCGGTCGCTAGCCACCATGGTGAGCAAGGGCGAGGAGCTGTTCACCG
GGGTGGTGCCCATCCTGGTCGAGCTGGACGGCGACGTAAACGGCCAC
AAGTTCAGCGTGTCCGGCGAGGGCGAGGGCGATGCCACCTACGGCAA
GCTGACCCTGAAGTTCATCTGCACCACCGGCAAGCTGCCCGTGCCCT
GGCCCACCCTCGTGACCACCCTGACCTACGGCGTGCAGTGCTTCAGC
CGCTACCCCGACCACATGAAGCAGCACGACTTCTTCAAGTCCGCCAT
GCCCGAAGGCTACGTCCAGGAGCGCACCATCTTCTTCAAGGACGACG
GCAACTACAAGACCCGCGCCGAGGTGAAGTTCGAGGGCGACACCCTG
GTGAACCGCATCGAGCTGAAGGGCATCGACTTCAAGGAGGACGGCAA
CATCCTGGGGCACAAGCTGGAGTACAACTACAACAGCCACAACGTCT
ATATCATGGCCGACAAGCAGAAGAACGGCATCAAGGTGAACTTCAAG
ATCCGCCACAACATCGAGGACGGCAGCGTGCAGCTCGCCGACCACTA
CCAGCAGAACACCCCCATCGGCGACGGCCCCGTGCTGCTGCCCGACA
ACCACTACCTGAGCACCCAGTCCGCCCTGAGCAAAGACCCCAACGAG
AAGCGCGATCACATGGTCCTGCTGGAGTTCGTGACCGCCGCCGGGAT
CACTCTCGGCATGGACGAGCTGTACAAGTAAcctgcaggcgccggcg
GCCGCTGGACCCCTCCACTCTGCTGTGGCCTTGAGAATCCTGATGGA
GCggGATTGCTGTCCCAAACTCccGCTCCATCGGATTCTCAAGGCTA
CAGTGAGGGGCGAGCTCCTTCTCCTGCGAGCTCGCTGATCAGCCTCG
ACTGTGCCTTCTAGTTGCCAGCCATCTGTTGTTTGCCCCTCCCCCGT
GCCTTCCTTGACCCTGGAAGGTGCCACTCCCACTGTCCTTTCCTAAT
AAAATGAGGAAATTGCATCGCATTGTCTGAGTAGGTGTCATTCTATT
CTGGGGGGTGGGGTGGGGCAGGACAGCAAGGGGGAGGATTGGGAAGA
CAATAGCAGGCATGCTGGGGATGCGGTGGGCTCTATGGAAGCTTGAA
TTCAGCTGACGTGCCTCGGACCGCTAGGAACCCCTAGTGATGGAGTT
GGCCACTCCCTCTCTGCGCGCTCGCTCGCTCACTGAGGCCGGGCGAC
CAAAGGTCGCCCGACGCCCGGGCTTTGCCCGGGCGGCCTCAGTGAGC
GAGCGAGCGCGCAG
Exemplary pITR.CAG.miR1-182_EGFP
sequence
(SEQ ID NO: 218)
CTGCGCGCTCGCTCGCTCACTGAGGCCGCCCGGGCGTCGGGCGACCT
TTGGTCGCCCGGCCTCAGTGAGCGAGCGAGCGCGCAGAGAGGGAGTG
GCCAACTCCATCACTAGGGGTTCCTGCGGCCGCACGCGTGACATTGA
TTATTGACTAGTTATTAATAGTAATCAATTACGGGGTCATTAGTTCA
TAGCCCATATATGGAGTTCCGCGTTACATAACTTACGGTAAATGGCC
CGCCTGGCTGACCGCCCAACGACCCCCGCCCATTGACGTCAATAATG
ACGTATGTTCCCATAGTAACGCCAATAGGGACTTTCCATTGACGTCA
ATGGGTGGACTATTTACGGTAAACTGCCCACTTGGCAGTACATCAAG
TGTATCATATGCCAAGTACGCCCCCTATTGACGTCAATGACGGTAAA
TGGCCCGCCTGGCATTATGCCCAGTACATGACCTTATGGGACTTTCC
TACTTGGCAGTACATCTACGTATTAGTCATCGCTATTACCATGGGTC
GAGGTGAGCCCCACGTTCTGCTTCACTCTCCCCATCTCCCCCCCCTC
CCCACCCCCAATTTTGTATTTATTTATTTTTTAATTATTTTGTGCAG
CGATGGGGGCGGGGGGGGGGGGGGCGCGCGCCAGGCGGGGCGGGGCG
GGGCGAGGGGCGGGGCGGGGCGAGGCGGAGAGGTGCGGCGGCAGCCA
ATCAGAGCGGCGCGCTCCGAAAGTTTCCTTTTATGGCGAGGCGGCGG
CGGCGGCGGCCCTATAAAAAGCGAAGCGCGCGGCGGGCGGGAGTCGC
TGCGTTGCCTTCGCCCCGTGCCCCGCTCCGCGCCGCCTCGCGCCGCC
CGCCCCGGCTCTGACTGACCGCGTTACTCCCACAGGTGAGCGGGCGG
GACGGCCCTTCTCCTCCGGGCTGTAATTAGCGCTTGGTTTAATGACG
GCTCGTTTCTTTTCTGTGGCTGCGTGAAAGCCTTAAAGGGCTCCGGG
AGGGCCCTTTGTGCGGGGGGGAGCGGCTCGGGGGGTGCGTGCGTGTG
TGTGTGCGTGGGGAGCGCCGCGTGCGGCCCGCGCTGCCCGGCGGCTG
TGAGCGCTGCGGGCGCGGCGCGGGGCTTTGTGCGCTCCGCGTGTGCG
CGAGGGGAGCGCGGCCGGGGGCGGTGCCCCGCGGTGCGGGGGGGCTG
CGAGGGGAACAAAGGCTGCGTGCGGGGTGTGTGCGTGGGGGGGTGAG
CAGGGGGTGTGGGCGCGGCGGTCGGGCTGTAACCCCCCCCTGCACCC
CCCTCCCCGAGTTGCTGAGCACGGCCCGGCTTCGGGTGCGGGGCTCC
GTGCGGGGCGTGGCGCGGGGCTCGCCGTGCCGGGCGGGGGGTGGCGG
CAGGTGGGGGTGCCGGGCGGGGCGGGGCCGCCTCGGGCCGGGGAGGG
CTCGGGGGAGGGGCGCGGCGGCCCCCGGAGCGCCGGCGCTCCCCAGC
TCCTGGGGGGAGCTGCTTGCCTCCCCCCGTTTTGAGAATCCTGATGG
AGCggACTGGTGAGGTAACAGGATCCGCCGTCCATCTTTATTCTCAC
TATGGGGCGAGGACTCAGCCGGCACCCTGTGCACAGCCAGCGAGGGT
CTAGAGCCTCTGCTAACCATGTTCATGCCTTCTTCTTTTTCCTACAG
CTCCTGGGCAACGTGCTGGTTATTGTGACCGGTCGCTAGCCACCATG
GTGAGCAAGGGCGAGGAGCTGTTCACCGGGGTGGTGCCCATCCTGGT
CGAGCTGGACGGCGACGTAAACGGCCACAAGTTCAGCGTGTCCGGCG
AGGGCGAGGGCGATGCCACCTACGGCAAGCTGACCCTGAAGTTCATC
TGCACCACCGGCAAGCTGCCCGTGCCCTGGCCCACCCTCGTGACCAC
CCTGACCTACGGCGTGCAGTGCTTCAGCCGCTACCCCGACCACATGA
AGCAGCACGACTTCTTCAAGTCCGCCATGCCCGAAGGCTACGTCCAG
GAGCGCACCATCTTCTTCAAGGACGACGGCAACTACAAGACCCGCGC
CGAGGTGAAGTTCGAGGGCGACACCCTGGTGAACCGCATCGAGCTGA
AGGGCATCGACTTCAAGGAGGACGGCAACATCCTGGGGCACAAGCTG
GAGTACAACTACAACAGCCACAACGTCTATATCATGGCCGACAAGCA
GAAGAACGGCATCAAGGTGAACTTCAAGATCCGCCACAACATCGAGG
ACGGCAGCGTGCAGCTCGCCGACCACTACCAGCAGAACACCCCCATC
GGCGACGGCCCCGTGCTGCTGCCCGACAACCACTACCTGAGCACCCA
GTCCGCCCTGAGCAAAGACCCCAACGAGAAGCGCGATCACATGGTCC
TGCTGGAGTTCGTGACCGCCGCCGGGATCACTCTCGGCATGGACGAG
CTGTACAAGTAAcctgcaggcgccggcgCTCCCCAGCTCCTGGGGGG
AGCTGCTTGCCTCCCCCCGTTTTGAGAATCCTGATGGAGCggACTGG
TGAGGTAACAGGATCCGCCGTCCATCTTTATTCTCACTATGGGGCGA
GGACTCAGCCGGCACCCTGTGCACAGCCAGCGAGGGGAGCTCGCTGA
TCAGCCTCGACTGTGCCTTCTAGTTGCCAGCCATCTGTTGTTTGCCC
CTCCCCCGTGCCTTCCTTGACCCTGGAAGGTGCCACTCCCACTGTCC
TTTCCTAATAAAATGAGGAAATTGCATCGCATTGTCTGAGTAGGTGT
CATTCTATTCTGGGGGGTGGGGTGGGGCAGGACAGCAAGGGGGAGGA
TTGGGAAGACAATAGCAGGCATGCTGGGGATGCGGTGGGCTCTATGG
AAGCTTGAATTCAGCTGACGTGCCTCGGACCGCTAGGAACCCCTAGT
GATGGAGTTGGCCACTCCCTCTCTGCGCGCTCGCTCGCTCACTGAGG
CCGGGCGACCAAAGGTCGCCCGACGCCCGGGCTTTGCCCGGGCGGCC
TCAGTGAGCGAGCGAGCGCGCAG
Exemplary pITR.CAG.miR1-183_EGFP
sequence
(SEQ ID NO: 219)
CTGCGCGCTCGCTCGCTCACTGAGGCCGCCCGGGCGTCGGGCGACCT
TTGGTCGCCCGGCCTCAGTGAGCGAGCGAGCGCGCAGAGAGGGAGTG
GCCAACTCCATCACTAGGGGTTCCTGCGGCCGCACGCGTGACATTGA
TTATTGACTAGTTATTAATAGTAATCAATTACGGGGTCATTAGTTCA
TAGCCCATATATGGAGTTCCGCGTTACATAACTTACGGTAAATGGCC
CGCCTGGCTGACCGCCCAACGACCCCCGCCCATTGACGTCAATAATG
ACGTATGTTCCCATAGTAACGCCAATAGGGACTTTCCATTGACGTCA
ATGGGTGGACTATTTACGGTAAACTGCCCACTTGGCAGTACATCAAG
TGTATCATATGCCAAGTACGCCCCCTATTGACGTCAATGACGGTAAA
TGGCCCGCCTGGCATTATGCCCAGTACATGACCTTATGGGACTTTCC
TACTTGGCAGTACATCTACGTATTAGTCATCGCTATTACCATGGGTC
GAGGTGAGCCCCACGTTCTGCTTCACTCTCCCCATCTCCCCCCCCTC
CCCACCCCCAATTTTGTATTTATTTATTTTTTAATTATTTTGTGCAG
CGATGGGGGCGGGGGGGGGGGGGGCGCGCGCCAGGCGGGGCGGGGCG
GGGCGAGGGGCGGGGCGGGGCGAGGCGGAGAGGTGCGGCGGCAGCCA
ATCAGAGCGGCGCGCTCCGAAAGTTTCCTTTTATGGCGAGGCGGCGG
CGGCGGCGGCCCTATAAAAAGCGAAGCGCGCGGCGGGCGGGAGTCGC
TGCGTTGCCTTCGCCCCGTGCCCCGCTCCGCGCCGCCTCGCGCCGCC
CGCCCCGGCTCTGACTGACCGCGTTACTCCCACAGGTGAGCGGGCGG
GACGGCCCTTCTCCTCCGGGCTGTAATTAGCGCTTGGTTTAATGACG
GCTCGTTTCTTTTCTGTGGCTGCGTGAAAGCCTTAAAGGGCTCCGGG
AGGGCCCTTTGTGCGGGGGGGAGCGGCTCGGGGGGTGCGTGCGTGTG
TGTGTGCGTGGGGAGCGCCGCGTGCGGCCCGCGCTGCCCGGCGGCTG
TGAGCGCTGCGGGCGCGGCGCGGGGCTTTGTGCGCTCCGCGTGTGCG
CGAGGGGAGCGCGGCCGGGGGCGGTGCCCCGCGGTGCGGGGGGGCTG
CGAGGGGAACAAAGGCTGCGTGCGGGGTGTGTGCGTGGGGGGGTGAG
CAGGGGGTGTGGGCGCGGCGGTCGGGCTGTAACCCCCCCCTGCACCC
CCCTCCCCGAGTTGCTGAGCACGGCCCGGCTTCGGGTGCGGGGCTCC
GTGCGGGGCGTGGCGCGGGGCTCGCCGTGCCGGGCGGGGGGTGGCGG
CAGGTGGGGGTGCCGGGCGGGGCGGGGCCGCCTCGGGCCGGGGAGGG
CTCGGGGGAGGGGCGCGGCGGCCCCCGGAGCGCCGGCGAGGGTCGGC
AGGCCGCAGAGTGTGACTCCTGTTCTGTGTTTGAGAATCCTGATGGA
GCggGTGAACAGTCTCAGTCccGCTCCCAGGAAATGGTCAAAAACAG
AGCAGAGACAGATCCACGAGGGCCTCCGGAGCACCTTTCTAGAGCCT
CTGCTAACCATGTTCATGCCTTCTTCTTTTTCCTACAGCTCCTGGGC
AACGTGCTGGTTATTGTGACCGGTCGCTAGCCACCATGGTGAGCAAG
GGCGAGGAGCTGTTCACCGGGGTGGTGCCCATCCTGGTCGAGCTGGA
CGGCGACGTAAACGGCCACAAGTTCAGCGTGTCCGGCGAGGGCGAGG
GCGATGCCACCTACGGCAAGCTGACCCTGAAGTTCATCTGCACCACC
GGCAAGCTGCCCGTGCCCTGGCCCACCCTCGTGACCACCCTGACCTA
CGGCGTGCAGTGCTTCAGCCGCTACCCCGACCACATGAAGCAGCACG
ACTTCTTCAAGTCCGCCATGCCCGAAGGCTACGTCCAGGAGCGCACC
ATCTTCTTCAAGGACGACGGCAACTACAAGACCCGCGCCGAGGTGAA
GTTCGAGGGCGACACCCTGGTGAACCGCATCGAGCTGAAGGGCATCG
ACTTCAAGGAGGACGGCAACATCCTGGGGCACAAGCTGGAGTACAAC
TACAACAGCCACAACGTCTATATCATGGCCGACAAGCAGAAGAACGG
CATCAAGGTGAACTTCAAGATCCGCCACAACATCGAGGACGGCAGCG
TGCAGCTCGCCGACCACTACCAGCAGAACACCCCCATCGGCGACGGC
CCCGTGCTGCTGCCCGACAACCACTACCTGAGCACCCAGTCCGCCCT
GAGCAAAGACCCCAACGAGAAGCGCGATCACATGGTCCTGCTGGAGT
TCGTGACCGCCGCCGGGATCACTCTCGGCATGGACGAGCTGTACAAG
TAAcctgcaggcgccggcgAGGGTCGGCAGGCCGCAGAGTGTGACTC
CTGTTCTGTGTTTGAGAATCCTGATGGAGCggGTGAACAGTCTCAGT
CccGCTCCCAGGAAATGGTCAAAAACAGAGCAGAGACAGATCCACGA
GGGCCTCCGGAGCACCTTGAGCTCGCTGATCAGCCTCGACTGTGCCT
TCTAGTTGCCAGCCATCTGTTGTTTGCCCCTCCCCCGTGCCTTCCTT
GACCCTGGAAGGTGCCACTCCCACTGTCCTTTCCTAATAAAATGAGG
AAATTGCATCGCATTGTCTGAGTAGGTGTCATTCTATTCTGGGGGGT
GGGGTGGGGCAGGACAGCAAGGGGGAGGATTGGGAAGACAATAGCAG
GCATGCTGGGGATGCGGTGGGCTCTATGGAAGCTTGAATTCAGCTGA
CGTGCCTCGGACCGCTAGGAACCCCTAGTGATGGAGTTGGCCACTCC
CTCTCTGCGCGCTCGCTCGCTCACTGAGGCCGGGCGACCAAAGGTCG
CCCGACGCCCGGGCTTTGCCCGGGCGGCCTCAGTGAGCGAGCGAGCG
CGCAG
Exemplary pITR.CAG.miR1-335_EGFP
sequence
(SEQ ID NO: 220)
CTGCGCGCTCGCTCGCTCACTGAGGCCGCCCGGGCGTCGGGCGACCT
TTGGTCGCCCGGCCTCAGTGAGCGAGCGAGCGCGCAGAGAGGGAGTG
GCCAACTCCATCACTAGGGGTTCCTGCGGCCGCACGCGTGACATTGA
TTATTGACTAGTTATTAATAGTAATCAATTACGGGGTCATTAGTTCA
TAGCCCATATATGGAGTTCCGCGTTACATAACTTACGGTAAATGGCC
CGCCTGGCTGACCGCCCAACGACCCCCGCCCATTGACGTCAATAATG
ACGTATGTTCCCATAGTAACGCCAATAGGGACTTTCCATTGACGTCA
ATGGGTGGACTATTTACGGTAAACTGCCCACTTGGCAGTACATCAAG
TGTATCATATGCCAAGTACGCCCCCTATTGACGTCAATGACGGTAAA
TGGCCCGCCTGGCATTATGCCCAGTACATGACCTTATGGGACTTTCC
TACTTGGCAGTACATCTACGTATTAGTCATCGCTATTACCATGGGTC
GAGGTGAGCCCCACGTTCTGCTTCACTCTCCCCATCTCCCCCCCCTC
CCCACCCCCAATTTTGTATTTATTTATTTTTTAATTATTTTGTGCAG
CGATGGGGGCGGGGGGGGGGGGGGCGCGCGCCAGGCGGGGCGGGGCG
GGGCGAGGGGCGGGGCGGGGCGAGGCGGAGAGGTGCGGCGGCAGCCA
ATCAGAGCGGCGCGCTCCGAAAGTTTCCTTTTATGGCGAGGCGGCGG
CGGCGGCGGCCCTATAAAAAGCGAAGCGCGCGGCGGGCGGGAGTCGC
TGCGTTGCCTTCGCCCCGTGCCCCGCTCCGCGCCGCCTCGCGCCGCC
CGCCCCGGCTCTGACTGACCGCGTTACTCCCACAGGTGAGCGGGCGG
GACGGCCCTTCTCCTCCGGGCTGTAATTAGCGCTTGGTTTAATGACG
GCTCGTTTCTTTTCTGTGGCTGCGTGAAAGCCTTAAAGGGCTCCGGG
AGGGCCCTTTGTGCGGGGGGGAGCGGCTCGGGGGGTGCGTGCGTGTG
TGTGTGCGTGGGGAGCGCCGCGTGCGGCCCGCGCTGCCCGGCGGCTG
TGAGCGCTGCGGGCGCGGCGCGGGGCTTTGTGCGCTCCGCGTGTGCG
CGAGGGGAGCGCGGCCGGGGGCGGTGCCCCGCGGTGCGGGGGGGCTG
CGAGGGGAACAAAGGCTGCGTGCGGGGTGTGTGCGTGGGGGGGTGAG
CAGGGGGTGTGGGCGCGGCGGTCGGGCTGTAACCCCCCCCTGCACCC
CCCTCCCCGAGTTGCTGAGCACGGCCCGGCTTCGGGTGCGGGGCTCC
GTGCGGGGCGTGGCGCGGGGCTCGCCGTGCCGGGCGGGGGGTGGCGG
CAGGTGGGGGTGCCGGGCGGGGCGGGGCCGCCTCGGGCCGGGGAGGG
CTCGGGGGAGGGGCGCGGCGGCCCCCGGAGCGCCGGCGGAAACAGAT
TGGAAATGATTTGTTTTGAGCGGGGGTTGAGAATCCTGATGGAGCgg
GTTTGTCATAAACCccGCTCCCTCAGGATTCCCAACCTCCTCTCATT
TGCTATATTCAATTAAGTAATCTAGAGCCTCTGCTAACCATGTTCAT
GCCTTCTTCTTTTTCCTACAGCTCCTGGGCAACGTGCTGGTTATTGT
GACCGGTCGCTAGCCACCATGGTGAGCAAGGGCGAGGAGCTGTTCAC
CGGGGTGGTGCCCATCCTGGTCGAGCTGGACGGCGACGTAAACGGCC
ACAAGTTCAGCGTGTCCGGCGAGGGCGAGGGCGATGCCACCTACGGC
AAGCTGACCCTGAAGTTCATCTGCACCACCGGCAAGCTGCCCGTGCC
CTGGCCCACCCTCGTGACCACCCTGACCTACGGCGTGCAGTGCTTCA
GCCGCTACCCCGACCACATGAAGCAGCACGACTTCTTCAAGTCCGCC
ATGCCCGAAGGCTACGTCCAGGAGCGCACCATCTTCTTCAAGGACGA
CGGCAACTACAAGACCCGCGCCGAGGTGAAGTTCGAGGGCGACACCC
TGGTGAACCGCATCGAGCTGAAGGGCATCGACTTCAAGGAGGACGGC
AACATCCTGGGGCACAAGCTGGAGTACAACTACAACAGCCACAACGT
CTATATCATGGCCGACAAGCAGAAGAACGGCATCAAGGTGAACTTCA
AGATCCGCCACAACATCGAGGACGGCAGCGTGCAGCTCGCCGACCAC
TACCAGCAGAACACCCCCATCGGCGACGGCCCCGTGCTGCTGCCCGA
CAACCACTACCTGAGCACCCAGTCCGCCCTGAGCAAAGACCCCAACG
AGAAGCGCGATCACATGGTCCTGCTGGAGTTCGTGACCGCCGCCGGG
ATCACTCTCGGCATGGACGAGCTGTACAAGTAAcctgcaggcgccgg
cgGAAACAGATTGGAAATGATTTGTTTTGAGCGGGGGTTGAGAATCC
TGATGGAGCggGTTTGTCATAAACCccGCTCCCTCAGGATTCCCAAC
CTCCTCTCATTTGCTATATTCAATTAAGTAAGAGCTCGCTGATCAGC
CTCGACTGTGCCTTCTAGTTGCCAGCCATCTGTTGTTTGCCCCTCCC
CCGTGCCTTCCTTGACCCTGGAAGGTGCCACTCCCACTGTCCTTTCC
TAATAAAATGAGGAAATTGCATCGCATTGTCTGAGTAGGTGTCATTC
TATTCTGGGGGGTGGGGTGGGGCAGGACAGCAAGGGGGAGGATTGGG
AAGACAATAGCAGGCATGCTGGGGATGCGGTGGGCTCTATGGAAGCT
TGAATTCAGCTGACGTGCCTCGGACCGCTAGGAACCCCTAGTGATGG
AGTTGGCCACTCCCTCTCTGCGCGCTCGCTCGCTCACTGAGGCCGGG
CGACCAAAGGTCGCCCGACGCCCGGGCTTTGCCCGGGCGGCCTCAGT
GAGCGAGCGAGCGCGCAG
Exemplary pITR.CAG.miR1-451_EGFP
sequence
(SEQ ID NO: 221)
CTGCGCGCTCGCTCGCTCACTGAGGCCGCCCGGGCGTCGGGCGACCT
TTGGTCGCCCGGCCTCAGTGAGCGAGCGAGCGCGCAGAGAGGGAGTG
GCCAACTCCATCACTAGGGGTTCCTGCGGCCGCACGCGTGACATTGA
TTATTGACTAGTTATTAATAGTAATCAATTACGGGGTCATTAGTTCA
TAGCCCATATATGGAGTTCCGCGTTACATAACTTACGGTAAATGGCC
CGCCTGGCTGACCGCCCAACGACCCCCGCCCATTGACGTCAATAATG
ACGTATGTTCCCATAGTAACGCCAATAGGGACTTTCCATTGACGTCA
ATGGGTGGACTATTTACGGTAAACTGCCCACTTGGCAGTACATCAAG
TGTATCATATGCCAAGTACGCCCCCTATTGACGTCAATGACGGTAAA
TGGCCCGCCTGGCATTATGCCCAGTACATGACCTTATGGGACTTTCC
TACTTGGCAGTACATCTACGTATTAGTCATCGCTATTACCATGGGTC
GAGGTGAGCCCCACGTTCTGCTTCACTCTCCCCATCTCCCCCCCCTC
CCCACCCCCAATTTTGTATTTATTTATTTTTTAATTATTTTGTGCAG
CGATGGGGGCGGGGGGGGGGGGGGCGCGCGCCAGGCGGGGCGGGGCG
GGGCGAGGGGCGGGGCGGGGCGAGGCGGAGAGGTGCGGCGGCAGCCA
ATCAGAGCGGCGCGCTCCGAAAGTTTCCTTTTATGGCGAGGCGGCGG
CGGCGGCGGCCCTATAAAAAGCGAAGCGCGCGGCGGGCGGGAGTCGC
TGCGTTGCCTTCGCCCCGTGCCCCGCTCCGCGCCGCCTCGCGCCGCC
CGCCCCGGCTCTGACTGACCGCGTTACTCCCACAGGTGAGCGGGCGG
GACGGCCCTTCTCCTCCGGGCTGTAATTAGCGCTTGGTTTAATGACG
GCTCGTTTCTTTTCTGTGGCTGCGTGAAAGCCTTAAAGGGCTCCGGG
AGGGCCCTTTGTGCGGGGGGGAGCGGCTCGGGGGGTGCGTGCGTGTG
TGTGTGCGTGGGGAGCGCCGCGTGCGGCCCGCGCTGCCCGGCGGCTG
TGAGCGCTGCGGGCGCGGCGCGGGGCTTTGTGCGCTCCGCGTGTGCG
CGAGGGGAGCGCGGCCGGGGGCGGTGCCCCGCGGTGCGGGGGGGCTG
CGAGGGGAACAAAGGCTGCGTGCGGGGTGTGTGCGTGGGGGGGTGAG
CAGGGGGTGTGGGCGCGGCGGTCGGGCTGTAACCCCCCCCTGCACCC
CCCTCCCCGAGTTGCTGAGCACGGCCCGGCTTCGGGTGCGGGGCTCC
GTGCGGGGCGTGGCGCGGGGCTCGCCGTGCCGGGCGGGGGGTGGCGG
CAGGTGGGGGTGCCGGGCGGGGCGGGGCCGCCTCGGGCCGGGGAGGG
CTCGGGGGAGGGGCGCGGCGGCCCCCGGAGCGCCGGCGTGACTGCCA
GGGCACTTGGGAATGGCAAGGTTGAGAATCCTGATGGAGCGGATTCC
ATCAGGATTCTCAGTCTTGCTATACCCAGAAAACGTGCCAGGAAGAT
CTAGAGCCTCTGCTAACCATGTTCATGCCTTCTTCTTTTTCCTACAG
CTCCTGGGCAACGTGCTGGTTATTGTGACCGGTCGCTAGCCACCATG
GTGAGCAAGGGCGAGGAGCTGTTCACCGGGGTGGTGCCCATCCTGGT
CGAGCTGGACGGCGACGTAAACGGCCACAAGTTCAGCGTGTCCGGCG
AGGGCGAGGGCGATGCCACCTACGGCAAGCTGACCCTGAAGTTCATC
TGCACCACCGGCAAGCTGCCCGTGCCCTGGCCCACCCTCGTGACCAC
CCTGACCTACGGCGTGCAGTGCTTCAGCCGCTACCCCGACCACATGA
AGCAGCACGACTTCTTCAAGTCCGCCATGCCCGAAGGCTACGTCCAG
GAGCGCACCATCTTCTTCAAGGACGACGGCAACTACAAGACCCGCGC
CGAGGTGAAGTTCGAGGGCGACACCCTGGTGAACCGCATCGAGCTGA
AGGGCATCGACTTCAAGGAGGACGGCAACATCCTGGGGCACAAGCTG
GAGTACAACTACAACAGCCACAACGTCTATATCATGGCCGACAAGCA
GAAGAACGGCATCAAGGTGAACTTCAAGATCCGCCACAACATCGAGG
ACGGCAGCGTGCAGCTCGCCGACCACTACCAGCAGAACACCCCCATC
GGCGACGGCCCCGTGCTGCTGCCCGACAACCACTACCTGAGCACCCA
GTCCGCCCTGAGCAAAGACCCCAACGAGAAGCGCGATCACATGGTCC
TGCTGGAGTTCGTGACCGCCGCCGGGATCACTCTCGGCATGGACGAG
CTGTACAAGTAACCTGCAGGCGCCGGCGTGACTGCCAGGGCACTTGG
GAATGGCAAGGTTGAGAATCCTGATGGAGCGGATTCCATCAGGATTC
TCAGTCTTGCTATACCCAGAAAACGTGCCAGGAAGAGAGCTCGCTGA
TCAGCCTCGACTGTGCCTTCTAGTTGCCAGCCATCTGTTGTTTGCCC
CTCCCCCGTGCCTTCCTTGACCCTGGAAGGTGCCACTCCCACTGTCC
TTTCCTAATAAAATGAGGAAATTGCATCGCATTGTCTGAGTAGGTGT
CATTCTATTCTGGGGGGTGGGGTGGGGCAGGACAGCAAGGGGGAGGA
TTGGGAAGACAATAGCAGGCATGCTGGGGATGCGGTGGGCTCTATGG
AAGCTTGAATTCAGCTGACGTGCCTCGGACCGCTAGGAACCCCTAGT
GATGGAGTTGGCCACTCCCTCTCTGCGCGCTCGCTCGCTCACTGAGG
CCGGGCGACCAAAGGTCGCCCGACGCCCGGGCTTTGCCCGGGCGGCC
TCAGTGAGCGAGCGAGCGCGCAG
Exemplary pITR.CAG.miR 2NNNNN_eGFP
sequence
(SEQ ID NO: 222)
CTGCGCGCTCGCTCGCTCACTGAGGCCGCCCGGGCGTCGGGCGACCT
TTGGTCGCCCGGCCTCAGTGAGCGAGCGAGCGCGCAGAGAGGGAGTG
GCCAACTCCATCACTAGGGGTTCCTGCGGCCGCACGCGTGACATTGA
TTATTGACTAGTTATTAATAGTAATCAATTACGGGGTCATTAGTTCA
TAGCCCATATATGGAGTTCCGCGTTACATAACTTACGGTAAATGGCC
CGCCTGGCTGACCGCCCAACGACCCCCGCCCATTGACGTCAATAATG
ACGTATGTTCCCATAGTAACGCCAATAGGGACTTTCCATTGACGTCA
ATGGGTGGACTATTTACGGTAAACTGCCCACTTGGCAGTACATCAAG
TGTATCATATGCCAAGTACGCCCCCTATTGACGTCAATGACGGTAAA
TGGCCCGCCTGGCATTATGCCCAGTACATGACCTTATGGGACTTTCC
TACTTGGCAGTACATCTACGTATTAGTCATCGCTATTACCATGGGTC
GAGGTGAGCCCCACGTTCTGCTTCACTCTCCCCATCTCCCCCCCCTC
CCCACCCCCAATTTTGTATTTATTTATTTTTTAATTATTTTGTGCAG
CGATGGGGGCGGGGGGGGGGGGGGCGCGCGCCAGGCGGGGCGGGGCG
GGGCGAGGGGCGGGGCGGGGCGAGGCGGAGAGGTGCGGCGGCAGCCA
ATCAGAGCGGCGCGCTCCGAAAGTTTCCTTTTATGGCGAGGCGGCGG
CGGCGGCGGCCCTATAAAAAGCGAAGCGCGCGGCGGGCGGGAGTCGC
TGCGTTGCCTTCGCCCCGTGCCCCGCTCCGCGCCGCCTCGCGCCGCC
CGCCCCGGCTCTGACTGACCGCGTTACTCCCACAGGTGAGCGGGCGG
GACGGCCCTTCTCCTCCGGGCTGTAATTAGCGCTTGGTTTAATGACG
GCTCGTTTCTTTTCTGTGGCTGCGTGAAAGCCTTAAAGGGCTCCGGG
AGGGCCCTTTGTGCGGGGGGGAGCGGCTCGGGGGGTGCGTGCGTGTG
TGTGTGCGTGGGGAGCGCCGCGTGCGGCCCGCGCTGCCCGGCGGCTG
TGAGCGCTGCGGGCGCGGCGCGGGGCTTTGTGCGCTCCGCGTGTGCG
CGAGGGGAGCGCGGCCGGGGGCGGTGCCCCGCGGTGCGGGGGGGCTG
CGAGGGGAACAAAGGCTGCGTGCGGGGTGTGTGCGTGGGGGGGTGAG
CAGGGGGTGTGGGCGCGGCGGTCGGGCTGTAACCCCCCCCTGCACCC
CCCTCCCCGAGTTGCTGAGCACGGCCCGGCTTCGGGTGCGGGGCTCC
GTGCGGGGCGTGGCGCGGGGCTCGCCGTGCCGGGCGGGGGGTGGCGG
CAGGTGGGGGTGCCGGGCGGGGCGGGGCCGCCTCGGGCCGGGGAGGG
CTCGGGGGAGGGGCGCGGCGGCCCCCGGAGCGCCGGCGNNNNNTCTA
GAGCCTCTGCTAACCATGTTCATGCCTTCTTCTTTTTCCTACAGCTC
CTGGGCAACGTGCTGGTTATTGTGACCGGTCGCTAGCCACCATGGTG
AGCAAGGGCGAGGAGCTGTTCACCGGGGTGGTGCCCATCCTGGTCGA
GCTGGACGGCGACGTAAACGGCCACAAGTTCAGCGTGTCCGGCGAGG
GCGAGGGCGATGCCACCTACGGCAAGCTGACCCTGAAGTTCATCTGC
ACCACCGGCAAGCTGCCCGTGCCCTGGCCCACCCTCGTGACCACCCT
GACCTACGGCGTGCAGTGCTTCAGCCGCTACCCCGACCACATGAAGC
AGCACGACTTCTTCAAGTCCGCCATGCCCGAAGGCTACGTCCAGGAG
CGCACCATCTTCTTCAAGGACGACGGCAACTACAAGACCCGCGCCGA
GGTGAAGTTCGAGGGCGACACCCTGGTGAACCGCATCGAGCTGAAGG
GCATCGACTTCAAGGAGGACGGCAACATCCTGGGGCACAAGCTGGAG
TACAACTACAACAGCCACAACGTCTATATCATGGCCGACAAGCAGAA
GAACGGCATCAAGGTGAACTTCAAGATCCGCCACAACATCGAGGACG
GCAGCGTGCAGCTCGCCGACCACTACCAGCAGAACACCCCCATCGGC
GACGGCCCCGTGCTGCTGCCCGACAACCACTACCTGAGCACCCAGTC
CGCCCTGAGCAAAGACCCCAACGAGAAGCGCGATCACATGGTCCTGC
TGGAGTTCGTGACCGCCGCCGGGATCACTCTCGGCATGGACGAGCTG
TACAAGTAACCTGCAGGCGCCGGCGNNNNNGAGCTCGCTGATCAGCC
TCGACTGTGCCTTCTAGTTGCCAGCCATCTGTTGTTTGCCCCTCCCC
CGTGCCTTCCTTGACCCTGGAAGGTGCCACTCCCACTGTCCTTTCCT
AATAAAATGAGGAAATTGCATCGCATTGTCTGAGTAGGTGTCATTCT
ATTCTGGGGGGTGGGGTGGGGCAGGACAGCAAGGGGGAGGATTGGGA
AGACAATAGCAGGCATGCTGGGGATGCGGTGGGCTCTATGGAAGCTT
GAATTCAGCTGACGTGCCTCGGACCGCTAGGAACCCCTAGTGATGGA
GTTGGCCACTCCCTCTCTGCGCGCTCGCTCGCTCACTGAGGCCGGGC
GACCAAAGGTCGCCCGACGCCCGGGCTTTGCCCGGGCGGCCTCAGTG
AGCGAGCGAGCGCGCAG
Exemplary pITR.CAG.miR_5NNNNN_eGFP
sequence
(SEQ ID NO: 223)
CTGCGCGCTCGCTCGCTCACTGAGGCCGCCCGGGCGTCGGGCGACCT
TTGGTCGCCCGGCCTCAGTGAGCGAGCGAGCGCGCAGAGAGGGAGTG
GCCAACTCCATCACTAGGGGTTCCTGCGGCCGCACGCGTGACATTGA
TTATTGACTAGTTATTAATAGTAATCAATTACGGGGTCATTAGTTCA
TAGCCCATATATGGAGTTCCGCGTTACATAACTTACGGTAAATGGCC
CGCCTGGCTGACCGCCCAACGACCCCCGCCCATTGACGTCAATAATG
ACGTATGTTCCCATAGTAACGCCAATAGGGACTTTCCATTGACGTCA
ATGGGTGGACTATTTACGGTAAACTGCCCACTTGGCAGTACATCAAG
TGTATCATATGCCAAGTACGCCCCCTATTGACGTCAATGACGGTAAA
TGGCCCGCCTGGCATTATGCCCAGTACATGACCTTATGGGACTTTCC
TACTTGGCAGTACATCTACGTATTAGTCATCGCTATTACCATGGGTC
GAGGTGAGCCCCACGTTCTGCTTCACTCTCCCCATCTCCCCCCCCTC
CCCACCCCCAATTTTGTATTTATTTATTTTTTAATTATTTTGTGCAG
CGATGGGGGCGGGGGGGGGGGGGGCGCGCGCCAGGCGGGGCGGGGCG
GGGCGAGGGGCGGGGCGGGGCGAGGCGGAGAGGTGCGGCGGCAGCCA
ATCAGAGCGGCGCGCTCCGAAAGTTTCCTTTTATGGCGAGGCGGCGG
CGGCGGCGGCCCTATAAAAAGCGAAGCGCGCGGCGGGCGGGAGTCGC
TGCGTTGCCTTCGCCCCGTGCCCCGCTCCGCGCCGCCTCGCGCCGCC
CGCCCCGGCTCTGACTGACCGCGTTACTCCCACAGGTGAGCGGGCGG
GACGGCCCTTCTCCTCCGGGCTGTAATTAGCGCTTGGTTTAATGACG
GCTCGTTTCTTTTCTGTGGCTGCGTGAAAGCCTTAAAGGGCTCCGGG
AGGGCCCTTTGTGCGGGGGGGAGCGGCTCGGGGGGTGCGTGCGTGTG
TGTGTGCGTGGGGAGCGCCGCGTGCGGCCCGCGCTGCCCGGCGGCTG
TGAGCGCTGCGGGCGCGGCGCGGGGCTTTGTGCGCTCCGCGTGTGCG
CGAGGGGAGCGCGGCCGGGGGCGGTGCCCCGCGGTGCGGGGGGGCTG
CGAGGGGAACAAAGGCTGCGTGCGGGGTGTGTGCGTGGGGGGGTGAG
CAGGGGGTGTGGGCGCGGCGGTCGGGCTGTAACCCCCCCCTGCACCC
CCCTCCCCGAGTTGCTGAGCACGGCCCGGCTTCGGGTGCGGGGCTCC
GTGCGGGGCGTGGCGCGGGGCTCGCCGTGCCGGGCGGGGGGTGGCGG
CAGGTGGGGGTGCCGGGCGGGGCGGGGCCGCCTCGGGCCGGGGAGGG
CTCGGGGGAGGGGCGCGGCGGCCCCCGGAGCGCCGGCGNNNNNTCTA
GAGCCTCTGCTAACCATGTTCATGCCTTCTTCTTTTTCCTACAGCTC
CTGGGCAACGTGCTGGTTATTGTGACCGGTCGCTAGCCACCATGGTG
AGCAAGGGCGAGGAGCTGTTCACCGGGGTGGTGCCCATCCTGGTCGA
GCTGGACGGCGACGTAAACGGCCACAAGTTCAGCGTGTCCGGCGAGG
GCGAGGGCGATGCCACCTACGGCAAGCTGACCCTGAAGTTCATCTGC
ACCACCGGCAAGCTGCCCGTGCCCTGGCCCACCCTCGTGACCACCCT
GACCTACGGCGTGCAGTGCTTCAGCCGCTACCCCGACCACATGAAGC
AGCACGACTTCTTCAAGTCCGCCATGCCCGAAGGCTACGTCCAGGAG
CGCACCATCTTCTTCAAGGACGACGGCAACTACAAGACCCGCGCCGA
GGTGAAGTTCGAGGGCGACACCCTGGTGAACCGCATCGAGCTGAAGG
GCATCGACTTCAAGGAGGACGGCAACATCCTGGGGCACAAGCTGGAG
TACAACTACAACAGCCACAACGTCTATATCATGGCCGACAAGCAGAA
GAACGGCATCAAGGTGAACTTCAAGATCCGCCACAACATCGAGGACG
GCAGCGTGCAGCTCGCCGACCACTACCAGCAGAACACCCCCATCGGC
GACGGCCCCGTGCTGCTGCCCGACAACCACTACCTGAGCACCCAGTC
CGCCCTGAGCAAAGACCCCAACGAGAAGCGCGATCACATGGTCCTGC
TGGAGTTCGTGACCGCCGCCGGGATCACTCTCGGCATGGACGAGCTG
TACAAGTAAGAGCTCGCTGATCAGCCTCGACTGTGCCTTCTAGTTGC
CAGCCATCTGTTGTTTGCCCCTCCCCCGTGCCTTCCTTGACCCTGGA
AGGTGCCACTCCCACTGTCCTTTCCTAATAAAATGAGGAAATTGCAT
CGCATTGTCTGAGTAGGTGTCATTCTATTCTGGGGGGTGGGGTGGGG
CAGGACAGCAAGGGGGAGGATTGGGAAGACAATAGCAGGCATGCTGG
GGATGCGGTGGGCTCTATGGAAGCTTGAATTCAGCTGACGTGCCTCG
GACCGCTAGGAACCCCTAGTGATGGAGTTGGCCACTCCCTCTCTGCG
CGCTCGCTCGCTCACTGAGGCCGGGCGACCAAAGGTCGCCCGACGCC
CGGGCTTTGCCCGGGCGGCCTCAGTGAGCGAGCGAGCGCGCAG
Exemplary pITR.CAG.miR 3′NNNNN_eGFP
sequence
(SEQ ID NO: 224)
CTGCGCGCTCGCTCGCTCACTGAGGCCGCCCGGGCGTCGGGCGACCT
TTGGTCGCCCGGCCTCAGTGAGCGAGCGAGCGCGCAGAGAGGGAGTG
GCCAACTCCATCACTAGGGGTTCCTGCGGCCGCACGCGTGACATTGA
TTATTGACTAGTTATTAATAGTAATCAATTACGGGGTCATTAGTTCA
TAGCCCATATATGGAGTTCCGCGTTACATAACTTACGGTAAATGGCC
CGCCTGGCTGACCGCCCAACGACCCCCGCCCATTGACGTCAATAATG
ACGTATGTTCCCATAGTAACGCCAATAGGGACTTTCCATTGACGTCA
ATGGGTGGACTATTTACGGTAAACTGCCCACTTGGCAGTACATCAAG
TGTATCATATGCCAAGTACGCCCCCTATTGACGTCAATGACGGTAAA
TGGCCCGCCTGGCATTATGCCCAGTACATGACCTTATGGGACTTTCC
TACTTGGCAGTACATCTACGTATTAGTCATCGCTATTACCATGGGTC
GAGGTGAGCCCCACGTTCTGCTTCACTCTCCCCATCTCCCCCCCCTC
CCCACCCCCAATTTTGTATTTATTTATTTTTTAATTATTTTGTGCAG
CGATGGGGGCGGGGGGGGGGGGGGCGCGCGCCAGGCGGGGCGGGGCG
GGGCGAGGGGCGGGGCGGGGCGAGGCGGAGAGGTGCGGCGGCAGCCA
ATCAGAGCGGCGCGCTCCGAAAGTTTCCTTTTATGGCGAGGCGGCGG
CGGCGGCGGCCCTATAAAAAGCGAAGCGCGCGGCGGGCGGGAGTCGC
TGCGTTGCCTTCGCCCCGTGCCCCGCTCCGCGCCGCCTCGCGCCGCC
CGCCCCGGCTCTGACTGACCGCGTTACTCCCACAGGTGAGCGGGCGG
GACGGCCCTTCTCCTCCGGGCTGTAATTAGCGCTTGGTTTAATGACG
GCTCGTTTCTTTTCTGTGGCTGCGTGAAAGCCTTAAAGGGCTCCGGG
AGGGCCCTTTGTGCGGGGGGGAGCGGCTCGGGGGGTGCGTGCGTGTG
TGTGTGCGTGGGGAGCGCCGCGTGCGGCCCGCGCTGCCCGGCGGCTG
TGAGCGCTGCGGGCGCGGCGCGGGGCTTTGTGCGCTCCGCGTGTGCG
CGAGGGGAGCGCGGCCGGGGGCGGTGCCCCGCGGTGCGGGGGGGCTG
CGAGGGGAACAAAGGCTGCGTGCGGGGTGTGTGCGTGGGGGGGTGAG
CAGGGGGTGTGGGCGCGGCGGTCGGGCTGTAACCCCCCCCTGCACCC
CCCTCCCCGAGTTGCTGAGCACGGCCCGGCTTCGGGTGCGGGGCTCC
GTGCGGGGCGTGGCGCGGGGCTCGCCGTGCCGGGCGGGGGGTGGCGG
CAGGTGGGGGTGCCGGGCGGGGCGGGGCCGCCTCGGGCCGGGGAGGG
CTCGGGGGAGGGGCGCGGCGGCCCCCGGAGCGCCGGCGGCTGTCGAG
GCGCGGCGAGCCGCAGCCATTGCCTTTTATGGTAATCGTGCGAGAGG
GCGCAGGGACTTCCTTTGTCCCAAATCTGTGCGGAGCCGAAATCTGG
GAGGCGCCGCCGCACCCCCTCTAGCGGGCGCGGGGCGAAGCGGTGCG
GCGCCGGCAGGAAGGAAATGGGCGGGGAGGGCCTTCGTGCGTCGCCG
CGCCGCCGTCCCCTTCTCCCTCTCCAGCCTCGGGGCTGTCCGCGGGG
GGACGGCTGCCTTCGGGGGGGACGGGGCAGGGCGGGGTTCGGCTTCT
GGCGTGTGACCGGCGGCTCTAGAGCCTCTGCTAACCATGTTCATGCC
TTCTTCTTTTTCCTACAGCTCCTGGGCAACGTGCTGGTTATTGTGAC
CGGTCGCTAGCCACCATGGTGAGCAAGGGCGAGGAGCTGTTCACCGG
GGTGGTGCCCATCCTGGTCGAGCTGGACGGCGACGTAAACGGCCACA
AGTTCAGCGTGTCCGGCGAGGGCGAGGGCGATGCCACCTACGGCAAG
CTGACCCTGAAGTTCATCTGCACCACCGGCAAGCTGCCCGTGCCCTG
GCCCACCCTCGTGACCACCCTGACCTACGGCGTGCAGTGCTTCAGCC
GCTACCCCGACCACATGAAGCAGCACGACTTCTTCAAGTCCGCCATG
CCCGAAGGCTACGTCCAGGAGCGCACCATCTTCTTCAAGGACGACGG
CAACTACAAGACCCGCGCCGAGGTGAAGTTCGAGGGCGACACCCTGG
TGAACCGCATCGAGCTGAAGGGCATCGACTTCAAGGAGGACGGCAAC
ATCCTGGGGCACAAGCTGGAGTACAACTACAACAGCCACAACGTCTA
TATCATGGCCGACAAGCAGAAGAACGGCATCAAGGTGAACTTCAAGA
TCCGCCACAACATCGAGGACGGCAGCGTGCAGCTCGCCGACCACTAC
CAGCAGAACACCCCCATCGGCGACGGCCCCGTGCTGCTGCCCGACAA
CCACTACCTGAGCACCCAGTCCGCCCTGAGCAAAGACCCCAACGAGA
AGCGCGATCACATGGTCCTGCTGGAGTTCGTGACCGCCGCCGGGATC
ACTCTCGGCATGGACGAGCTGTACAAGTAANNNNNGAGCTCGCTGAT
CAGCCTCGACTGTGCCTTCTAGTTGCCAGCCATCTGTTGTTTGCCCC
TCCCCCGTGCCTTCCTTGACCCTGGAAGGTGCCACTCCCACTGTCCT
TTCCTAATAAAATGAGGAAATTGCATCGCATTGTCTGAGTAGGTGTC
ATTCTATTCTGGGGGGTGGGGTGGGGCAGGACAGCAAGGGGGAGGAT
TGGGAAGACAATAGCAGGCATGCTGGGGATGCGGTGGGCTCTATGGA
AGCTTGAATTCAGCTGACGTGCCTCGGACCGCTAGGAACCCCTAGTG
ATGGAGTTGGCCACTCCCTCTCTGCGCGCTCGCTCGCTCACTGAGGC
CGGGCGACCAAAGGTCGCCCGACGCCCGGGCTTTGCCCGGGCGGCCT
CAGTGAGCGAGCGAGCGCGCAG

Exemplary miRNA Construct Sequences (Part 3)

Exemplary pITR.CAG.miR1-155_EGFP sequence
(SEQ ID NO: 225)
CCTGCAGGCAGCTGCGCGCTCGCTCGCTCACTGAGGCCGCCCGGGCG
TCGGGCGACCTTTGGTCGCCCGGCCTCAGTGAGCGAGCGAGCGCGCA
GAGAGGGAGTGGCCAACTCCATCACTAGGGGTTCCTGCGGCCGCACG
CGTGACATTGATTATTGACTAGTTATTAATAGTAATCAATTACGGGG
TCATTAGTTCATAGCCCATATATGGAGTTCCGCGTTACATAACTTAC
GGTAAATGGCCCGCCTGGCTGACCGCCCAACGACCCCCGCCCATTGA
CGTCAATAATGACGTATGTTCCCATAGTAACGCCAATAGGGACTTTC
CATTGACGTCAATGGGTGGACTATTTACGGTAAACTGCCCACTTGGC
AGTACATCAAGTGTATCATATGCCAAGTACGCCCCCTATTGACGTCA
ATGACGGTAAATGGCCCGCCTGGCATTATGCCCAGTACATGACCTTA
TGGGACTTTCCTACTTGGCAGTACATCTACGTATTAGTCATCGCTAT
TACCATGGGTCGAGGTGAGCCCCACGTTCTGCTTCACTCTCCCCATC
TCCCCCCCCTCCCCACCCCCAATTTTGTATTTATTTATTTTTTAATT
ATTTTGTGCAGCGATGGGGGCGGGGGGGGGGGGGGCGCGCGCCAGGC
GGGGCGGGGCGGGGCGAGGGGCGGGGCGGGGCGAGGCGGAGAGGTGC
GGCGGCAGCCAATCAGAGCGGCGCGCTCCGAAAGTTTCCTTTTATGG
CGAGGCGGCGGCGGCGGCGGCCCTATAAAAAGCGAAGCGCGCGGCGG
GCGGGAGTCGCTGCGTTGCCTTCGCCCCGTGCCCCGCTCCGCGCCGC
CTCGCGCCGCCCGCCCCGGCTCTGACTGACCGCGTTACTCCCACAGG
TGAGCGGGCGGGACGGCCCTTCTCCTCCGGGCTGTAATTAGCGCTTG
GTTTAATGACGGCTCGTTTCTTTTCTGTGGCTGCGTGAAAGCCTTAA
AGGGCTCCGGGAGGGCCCTTTGTGCGGGGGGGAGCGGCTCGGGGGGT
GCGTGCGTGTGTGTGTGCGTGGGGAGCGCCGCGTGCGGCCCGCGCTG
CCCGGCGGCTGTGAGCGCTGCGGGCGCGGCGCGGGGCTTTGTGCGCT
CCGCGTGTGCGCGAGGGGAGCGCGGCCGGGGGCGGTGCCCCGCGGTG
CGGGGGGGCTGCGAGGGGAACAAAGGCTGCGTGCGGGGTGTGTGCGT
GGGGGGGTGAGCAGGGGGTGTGGGCGCGGCGGTCGGGCTGTAACCCC
CCCCTGCACCCCCCTCCCCGAGTTGCTGAGCACGGCCCGGCTTCGGG
TGCGGGGCTCCGTGCGGGGCGTGGCGCGGGGCTCGCCGTGCCGGGCG
GGGGGTGGCGGCAGGTGGGGGTGCCGGGCGGGGCGGGGCCGCCTCGG
GCCGGGGAGGGCTCGGGGGAGGGGCGCGGCGGCCCCCGGAGCGCCGG
CGTCCTCTGAGTGCTGAAGGCTTGCTGTAGGCTGTATGCTGTTGAGA
ATCCTGATGGAGCGGGTTTTTGCCTCCAACTGACCCGCTCATAGGAT
TCTCAACAGTGTATGATGCCTGTTACTAGCATTCACATGGATCTAGA
GCCTCTGCTAACCATGTTCATGCCTTCTTCTTTTTCCTACAGCTCCT
GGGCAACGTGCTGGTTATTGTGACCGGTCGCTAGCCACCATGGTGAG
CAAGGGCGAGGAGCTGTTCACCGGGGTGGTGCCCATCCTGGTCGAGC
TGGACGGCGACGTAAACGGCCACAAGTTCAGCGTGTCCGGCGAGGGC
GAGGGCGATGCCACCTACGGCAAGCTGACCCTGAAGTTCATCTGCAC
CACCGGCAAGCTGCCCGTGCCCTGGCCCACCCTCGTGACCACCCTGA
CCTACGGCGTGCAGTGCTTCAGCCGCTACCCCGACCACATGAAGCAG
CACGACTTCTTCAAGTCCGCCATGCCCGAAGGCTACGTCCAGGAGCG
CACCATCTTCTTCAAGGACGACGGCAACTACAAGACCCGCGCCGAGG
TGAAGTTCGAGGGCGACACCCTGGTGAACCGCATCGAGCTGAAGGGC
ATCGACTTCAAGGAGGACGGCAACATCCTGGGGCACAAGCTGGAGTA
CAACTACAACAGCCACAACGTCTATATCATGGCCGACAAGCAGAAGA
ACGGCATCAAGGTGAACTTCAAGATCCGCCACAACATCGAGGACGGC
AGCGTGCAGCTCGCCGACCACTACCAGCAGAACACCCCCATCGGCGA
CGGCCCCGTGCTGCTGCCCGACAACCACTACCTGAGCACCCAGTCCG
CCCTGAGCAAAGACCCCAACGAGAAGCGCGATCACATGGTCCTGCTG
GAGTTCGTGACCGCCGCCGGGATCACTCTCGGCATGGACGAGCTGTA
CAAGTAACCTGCAGGCGCCGGCGTCCTCTGAGTGCTGAAGGCTTGCT
GTAGGCTGTATGCTGTTGAGAATCCTGATGGAGCGGGTTTTTGCCTC
CAACTGACCCGCTCATAGGATTCTCAACAGTGTATGATGCCTGTTAC
TAGCATTCACATGGAGAGCTCGCTGATCAGCCTCGACTGTGCCTTCT
AGTTGCCAGCCATCTGTTGTTTGCCCCTCCCCCGTGCCTTCCTTGAC
CCTGGAAGGTGCCACTCCCACTGTCCTTTCCTAATAAAATGAGGAAA
TTGCATCGCATTGTCTGAGTAGGTGTCATTCTATTCTGGGGGGTGGG
GTGGGGCAGGACAGCAAGGGGGAGGATTGGGAAGACAATAGCAGGCA
TGCTGGGGATGCGGTGGGCTCTATGGAAGCTTGAATTCAGCTGACGT
GCCTCGGACCGCTAGGAACCCCTAGTGATGGAGTTGGCCACTCCCTC
TCTGCGCGCTCGCTCGCTCACTGAGGCCGGGCGACCAAAGGTCGCCC
GACGCCCGGGCTTTGCCCGGGCGGCCTCAGTGAGCGAGCGAGCGCGC
AG
Exemplary pITR.CAG.miR1i-155_EGFP sequence
(SEQ ID NO: 226)
CCTGCAGGCAGCTGCGCGCTCGCTCGCTCACTGAGGCCGCCCGGGCG
TCGGGCGACCTTTGGTCGCCCGGCCTCAGTGAGCGAGCGAGCGCGCA
GAGAGGGAGTGGCCAACTCCATCACTAGGGGTTCCTGCGGCCGCACG
CGTGACATTGATTATTGACTAGTTATTAATAGTAATCAATTACGGGG
TCATTAGTTCATAGCCCATATATGGAGTTCCGCGTTACATAACTTAC
GGTAAATGGCCCGCCTGGCTGACCGCCCAACGACCCCCGCCCATTGA
CGTCAATAATGACGTATGTTCCCATAGTAACGCCAATAGGGACTTTC
CATTGACGTCAATGGGTGGACTATTTACGGTAAACTGCCCACTTGGC
AGTACATCAAGTGTATCATATGCCAAGTACGCCCCCTATTGACGTCA
ATGACGGTAAATGGCCCGCCTGGCATTATGCCCAGTACATGACCTTA
TGGGACTTTCCTACTTGGCAGTACATCTACGTATTAGTCATCGCTAT
TACCATGGGTCGAGGTGAGCCCCACGTTCTGCTTCACTCTCCCCATC
TCCCCCCCCTCCCCACCCCCAATTTTGTATTTATTTATTTTTTAATT
ATTTTGTGCAGCGATGGGGGCGGGGGGGGGGGGGGCGCGCGCCAGGC
GGGGCGGGGCGGGGCGAGGGGCGGGGCGGGGCGAGGCGGAGAGGTGC
GGCGGCAGCCAATCAGAGCGGCGCGCTCCGAAAGTTTCCTTTTATGG
CGAGGCGGCGGCGGCGGCGGCCCTATAAAAAGCGAAGCGCGCGGCGG
GCGGGAGTCGCTGCGTTGCCTTCGCCCCGTGCCCCGCTCCGCGCCGC
CTCGCGCCGCCCGCCCCGGCTCTGACTGACCGCGTTACTCCCACAGG
TGAGCGGGCGGGACGGCCCTTCTCCTCCGGGCTGTAATTAGCGCTTG
GTTTAATGACGGCTCGTTTCTTTTCTGTGGCTGCGTGAAAGCCTTAA
AGGGCTCCGGGAGGGCCCTTTGTGCGGGGGGGAGCGGCTCGGGGGGT
GCGTGCGTGTGTGTGTGCGTGGGGAGCGCCGCGTGCGGCCCGCGCTG
CCCGGCGGCTGTGAGCGCTGCGGGCGCGGCGCGGGGCTTTGTGCGCT
CCGCGTGTGCGCGAGGGGAGCGCGGCCGGGGGCGGTGCCCCGCGGTG
CGGGGGGGCTGCGAGGGGAACAAAGGCTGCGTGCGGGGTGTGTGCGT
GGGGGGGTGAGCAGGGGGTGTGGGCGCGGCGGTCGGGCTGTAACCCC
CCCCTGCACCCCCCTCCCCGAGTTGCTGAGCACGGCCCGGCTTCGGG
TGCGGGGCTCCGTGCGGGGCGTGGCGCGGGGCTCGCCGTGCCGGGCG
GGGGGTGGCGGCAGGTGGGGGTGCCGGGCGGGGCGGGGCCGCCTCGG
GCCGGGGAGGGCTCGGGGGAGGGGCGCGGCGGCCCCCGGAGCGCCGG
CGTCCTCTGAGTGCTGAAGGCTTGCTGTAGGCTGTATGCTGTTGAGA
ATCCTGATGGAGCGGGTTTTTGCCTCCAACTGACCCGCTCATAGGAT
TCTCAACAGTGTATGATGCCTGTTACTAGCATTCACATGGATCTAGA
GCCTCTGCTAACCATGTTCATGCCTTCTTCTTTTTCCTACAGCTCCT
GGGCAACGTGCTGGTTATTGTGACCGGTCGCTAGCCACCATGGTGAG
CAAGGGCGAGGAGCTGTTCACCGGGGTGGTGCCCATCCTGGTCGAGC
TGGACGGCGACGTAAACGGCCACAAGTTCAGCGTGTCCGGCGAGGGC
GAGGGCGATGCCACCTACGGCAAGCTGACCCTGAAGTTCATCTGCAC
CACCGGCAAGCTGCCCGTGCCCTGGCCCACCCTCGTGACCACCCTGA
CCTACGGCGTGCAGTGCTTCAGCCGCTACCCCGACCACATGAAGCAG
CACGACTTCTTCAAGTCCGCCATGCCCGAAGGCTACGTCCAGGAGCG
CACCATCTTCTTCAAGGACGACGGCAACTACAAGACCCGCGCCGAGG
TGAAGTTCGAGGGCGACACCCTGGTGAACCGCATCGAGCTGAAGGGC
ATCGACTTCAAGGAGGACGGCAACATCCTGGGGCACAAGCTGGAGTA
CAACTACAACAGCCACAACGTCTATATCATGGCCGACAAGCAGAAGA
ACGGCATCAAGGTGAACTTCAAGATCCGCCACAACATCGAGGACGGC
AGCGTGCAGCTCGCCGACCACTACCAGCAGAACACCCCCATCGGCGA
CGGCCCCGTGCTGCTGCCCGACAACCACTACCTGAGCACCCAGTCCG
CCCTGAGCAAAGACCCCAACGAGAAGCGCGATCACATGGTCCTGCTG
GAGTTCGTGACCGCCGCCGGGATCACTCTCGGCATGGACGAGCTGTA
CAAGTAAGAGCTCGCTGATCAGCCTCGACTGTGCCTTCTAGTTGCCA
GCCATCTGTTGTTTGCCCCTCCCCCGTGCCTTCCTTGACCCTGGAAG
GTGCCACTCCCACTGTCCTTTCCTAATAAAATGAGGAAATTGCATCG
CATTGTCTGAGTAGGTGTCATTCTATTCTGGGGGGTGGGGTGGGGCA
GGACAGCAAGGGGGAGGATTGGGAAGACAATAGCAGGCATGCTGGGG
ATGCGGTGGGCTCTATGGAAGCTTGAATTCAGCTGACGTGCCTCGGA
CCGCTAGGAACCCCTAGTGATGGAGTTGGCCACTCCCTCTCTGCGCG
CTCGCTCGCTCACTGAGGCCGGGCGACCAAAGGTCGCCCGACGCCCG
GGCTTTGCCCGGGCGGCCTCAGTGAGCGAGCGAGCGCGCAG
Exemplary pITR.CAG.miR1u-155_EGFP sequence
(SEQ ID NO: 227)
CCTGCAGGCAGCTGCGCGCTCGCTCGCTCACTGAGGCCGCCCGGGCGT
CGGGCGACCTTTGGTCGCCCGGCCTCAGTGAGCGAGCGAGCGCGCAG
AGAGGGAGTGGCCAACTCCATCACTAGGGGTTCCTGCGGCCGCACGC
GTGACATTGATTATTGACTAGTTATTAATAGTAATCAATTACGGGGT
CATTAGTTCATAGCCCATATATGGAGTTCCGCGTTACATAACTTACG
GTAAATGGCCCGCCTGGCTGACCGCCCAACGACCCCCGCCCATTGAC
GTCAATAATGACGTATGTTCCCATAGTAACGCCAATAGGGACTTTCC
ATTGACGTCAATGGGTGGACTATTTACGGTAAACTGCCCACTTGGCA
GTACATCAAGTGTATCATATGCCAAGTACGCCCCCTATTGACGTCAA
TGACGGTAAATGGCCCGCCTGGCATTATGCCCAGTACATGACCTTAT
GGGACTTTCCTACTTGGCAGTACATCTACGTATTAGTCATCGCTATT
ACCATGGGTCGAGGTGAGCCCCACGTTCTGCTTCACTCTCCCCATCT
CCCCCCCCTCCCCACCCCCAATTTTGTATTTATTTATTTTTTAATTA
TTTTGTGCAGCGATGGGGGCGGGGGGGGGGGGGGCGCGCGCCAGGCG
GGGCGGGGCGGGGCGAGGGGCGGGGCGGGGCGAGGCGGAGAGGTGCG
GCGGCAGCCAATCAGAGCGGCGCGCTCCGAAAGTTTCCTTTTATGGC
GAGGCGGCGGCGGCGGCGGCCCTATAAAAAGCGAAGCGCGCGGCGGG
CGGGAGTCGCTGCGTTGCCTTCGCCCCGTGCCCCGCTCCGCGCCGCC
TCGCGCCGCCCGCCCCGGCTCTGACTGACCGCGTTACTCCCACAGGT
GAGCGGGCGGGACGGCCCTTCTCCTCCGGGCTGTAATTAGCGCTTGG
TTTAATGACGGCTCGTTTCTTTTCTGTGGCTGCGTGAAAGCCTTAAA
GGGCTCCGGGAGGGCCCTTTGTGCGGGGGGGAGCGGCTCGGGGGGTG
CGTGCGTGTGTGTGTGCGTGGGGAGCGCCGCGTGCGGCCCGCGCTGC
CCGGCGGCTGTGAGCGCTGCGGGCGCGGCGCGGGGCTTTGTGCGCTC
CGCGTGTGCGCGAGGGGAGCGCGGCCGGGGGCGGTGCCCCGCGGTGC
GGGGGGGCTGCGAGGGGAACAAAGGCTGCGTGCGGGGTGTGTGCGTG
GGGGGGTGAGCAGGGGGTGTGGGCGCGGCGGTCGGGCTGTAACCCCC
CCCTGCACCCCCCTCCCCGAGTTGCTGAGCACGGCCCGGCTTCGGGT
GCGGGGCTCCGTGCGGGGCGTGGCGCGGGGCTCGCCGTGCCGGGCGG
GGGGTGGCGGCAGGTGGGGGTGCCGGGCGGGGCGGGGCCGCCTCGGG
CCGGGGAGGGCTCGGGGGAGGGGCGCGGCGGCCCCCGGAGCGCCGGC
GGCTGTCGAGGCGCGGCGAGCCGCAGCCATTGCCTTTTATGGTAATC
GTGCGAGAGGGCGCAGGGACTTCCTTTGTCCCAAATCTGTGCGGAGC
CGAAATCTGGGAGGCGCCGCCGCACCCCCTCTAGCGGGCGCGGGGCG
AAGCGGTGCGGCGCCGGCAGGAAGGAAATGGGCGGGGAGGGCCTTCG
TGCGTCGCCGCGCCGCCGTCCCCTTCTCCCTCTCCAGCCTCGGGGCT
GTCCGCGGGGGGACGGCTGCCTTCGGGGGGGACGGGGCAGGGCGGGG
TTCGGCTTCTGGCGTGTGACCGGCGGCTCTAGAGCCTCTGCTAACCA
TGTTCATGCCTTCTTCTTTTTCCTACAGCTCCTGGGCAACGTGCTGG
TTATTGTGACCGGTCGCTAGCCACCATGGTGAGCAAGGGCGAGGAGC
TGTTCACCGGGGTGGTGCCCATCCTGGTCGAGCTGGACGGCGACGTA
AACGGCCACAAGTTCAGCGTGTCCGGCGAGGGCGAGGGCGATGCCAC
CTACGGCAAGCTGACCCTGAAGTTCATCTGCACCACCGGCAAGCTGC
CCGTGCCCTGGCCCACCCTCGTGACCACCCTGACCTACGGCGTGCAG
TGCTTCAGCCGCTACCCCGACCACATGAAGCAGCACGACTTCTTCAA
GTCCGCCATGCCCGAAGGCTACGTCCAGGAGCGCACCATCTTCTTCA
AGGACGACGGCAACTACAAGACCCGCGCCGAGGTGAAGTTCGAGGGC
GACACCCTGGTGAACCGCATCGAGCTGAAGGGCATCGACTTCAAGGA
GGACGGCAACATCCTGGGGCACAAGCTGGAGTACAACTACAACAGCC
ACAACGTCTATATCATGGCCGACAAGCAGAAGAACGGCATCAAGGTG
AACTTCAAGATCCGCCACAACATCGAGGACGGCAGCGTGCAGCTCGC
CGACCACTACCAGCAGAACACCCCCATCGGCGACGGCCCCGTGCTGC
TGCCCGACAACCACTACCTGAGCACCCAGTCCGCCCTGAGCAAAGAC
CCCAACGAGAAGCGCGATCACATGGTCCTGCTGGAGTTCGTGACCGC
CGCCGGGATCACTCTCGGCATGGACGAGCTGTACAAGTAACCTGCAG
GCGCCGGCGTCCTCTGAGTGCTGAAGGCTTGCTGTAGGCTGTATGCT
GTTGAGAATCCTGATGGAGCGGGTTTTTGCCTCCAACTGACCCGCTC
ATAGGATTCTCAACAGTGTATGATGCCTGTTACTAGCATTCACATGG
AGAGCTCGCTGATCAGCCTCGACTGTGCCTTCTAGTTGCCAGCCATC
TGTTGTTTGCCCCTCCCCCGTGCCTTCCTTGACCCTGGAAGGTGCCA
CTCCCACTGTCCTTTCCTAATAAAATGAGGAAATTGCATCGCATTGT
CTGAGTAGGTGTCATTCTATTCTGGGGGGTGGGGTGGGGCAGGACAG
CAAGGGGGAGGATTGGGAAGACAATAGCAGGCATGCTGGGGATGCGG
TGGGCTCTATGGAAGCTTGAATTCAGCTGACGTGCCTCGGACCGCTA
GGAACCCCTAGTGATGGAGTTGGCCACTCCCTCTCTGCGCGCTCGCT
CGCTCACTGAGGCCGGGCGACCAAAGGTCGCCCGACGCCCGGGCTTT
GCCCGGGCGGCCTCAGTGAGCGAGCGAGCGCGCAG
Exemplary pITR.CAG.miR2-155_EGFP sequence
(SEQ ID NO: 228)
CCTGCAGGCAGCTGCGCGCTCGCTCGCTCACTGAGGCCGCCCGGGCG
TCGGGCGACCTTTGGTCGCCCGGCCTCAGTGAGCGAGCGAGCGCGCA
GAGAGGGAGTGGCCAACTCCATCACTAGGGGTTCCTGCGGCCGCACG
CGTGACATTGATTATTGACTAGTTATTAATAGTAATCAATTACGGGG
TCATTAGTTCATAGCCCATATATGGAGTTCCGCGTTACATAACTTAC
GGTAAATGGCCCGCCTGGCTGACCGCCCAACGACCCCCGCCCATTGA
CGTCAATAATGACGTATGTTCCCATAGTAACGCCAATAGGGACTTTC
CATTGACGTCAATGGGTGGACTATTTACGGTAAACTGCCCACTTGGC
AGTACATCAAGTGTATCATATGCCAAGTACGCCCCCTATTGACGTCA
ATGACGGTAAATGGCCCGCCTGGCATTATGCCCAGTACATGACCTTA
TGGGACTTTCCTACTTGGCAGTACATCTACGTATTAGTCATCGCTAT
TACCATGGGTCGAGGTGAGCCCCACGTTCTGCTTCACTCTCCCCATC
TCCCCCCCCTCCCCACCCCCAATTTTGTATTTATTTATTTTTTAATT
ATTTTGTGCAGCGATGGGGGCGGGGGGGGGGGGGGCGCGCGCCAGGC
GGGGCGGGGCGGGGCGAGGGGCGGGGCGGGGCGAGGCGGAGAGGTGC
GGCGGCAGCCAATCAGAGCGGCGCGCTCCGAAAGTTTCCTTTTATGG
CGAGGCGGCGGCGGCGGCGGCCCTATAAAAAGCGAAGCGCGCGGCGG
GCGGGAGTCGCTGCGTTGCCTTCGCCCCGTGCCCCGCTCCGCGCCGC
CTCGCGCCGCCCGCCCCGGCTCTGACTGACCGCGTTACTCCCACAGG
TGAGCGGGCGGGACGGCCCTTCTCCTCCGGGCTGTAATTAGCGCTTG
GTTTAATGACGGCTCGTTTCTTTTCTGTGGCTGCGTGAAAGCCTTAA
AGGGCTCCGGGAGGGCCCTTTGTGCGGGGGGGAGCGGCTCGGGGGGT
GCGTGCGTGTGTGTGTGCGTGGGGAGCGCCGCGTGCGGCCCGCGCTG
CCCGGCGGCTGTGAGCGCTGCGGGCGCGGCGCGGGGCTTTGTGCGCT
CCGCGTGTGCGCGAGGGGAGCGCGGCCGGGGGCGGTGCCCCGCGGTG
CGGGGGGGCTGCGAGGGGAACAAAGGCTGCGTGCGGGGTGTGTGCGT
GGGGGGGTGAGCAGGGGGTGTGGGCGCGGCGGTCGGGCTGTAACCCC
CCCCTGCACCCCCCTCCCCGAGTTGCTGAGCACGGCCCGGCTTCGGG
TGCGGGGCTCCGTGCGGGGCGTGGCGCGGGGCTCGCCGTGCCGGGCG
GGGGGTGGCGGCAGGTGGGGGTGCCGGGCGGGGCGGGGCCGCCTCGG
GCCGGGGAGGGCTCGGGGGAGGGGCGCGGCGGCCCCCGGAGCGCCGG
CGTCCTCTGAGTGCTGAAGGCTTGCTGTAGGCTGTATGCTGTTGAGT
CTCAGAGATGCCCGGGTTTTTGCCTCCAACTGACTCGGGATTCTGAG
ACTCAACAGTGTATGATGCCTGTTACTAGCATTCACATGGATCTAGA
GCCTCTGCTAACCATGTTCATGCCTTCTTCTTTTTCCTACAGCTCCT
GGGCAACGTGCTGGTTATTGTGACCGGTCGCTAGCCACCATGGTGAG
CAAGGGCGAGGAGCTGTTCACCGGGGTGGTGCCCATCCTGGTCGAGC
TGGACGGCGACGTAAACGGCCACAAGTTCAGCGTGTCCGGCGAGGGC
GAGGGCGATGCCACCTACGGCAAGCTGACCCTGAAGTTCATCTGCAC
CACCGGCAAGCTGCCCGTGCCCTGGCCCACCCTCGTGACCACCCTGA
CCTACGGCGTGCAGTGCTTCAGCCGCTACCCCGACCACATGAAGCAG
CACGACTTCTTCAAGTCCGCCATGCCCGAAGGCTACGTCCAGGAGCG
CACCATCTTCTTCAAGGACGACGGCAACTACAAGACCCGCGCCGAGG
TGAAGTTCGAGGGCGACACCCTGGTGAACCGCATCGAGCTGAAGGGC
ATCGACTTCAAGGAGGACGGCAACATCCTGGGGCACAAGCTGGAGTA
CAACTACAACAGCCACAACGTCTATATCATGGCCGACAAGCAGAAGA
ACGGCATCAAGGTGAACTTCAAGATCCGCCACAACATCGAGGACGGC
AGCGTGCAGCTCGCCGACCACTACCAGCAGAACACCCCCATCGGCGA
CGGCCCCGTGCTGCTGCCCGACAACCACTACCTGAGCACCCAGTCCG
CCCTGAGCAAAGACCCCAACGAGAAGCGCGATCACATGGTCCTGCTG
GAGTTCGTGACCGCCGCCGGGATCACTCTCGGCATGGACGAGCTGTA
CAAGTAACCTGCAGGCGCCGGCGTCCTCTGAGTGCTGAAGGCTTGCT
GTAGGCTGTATGCTGTTGAGTCTCAGAGATGCCCGGGTTTTTGCCTC
CAACTGACTCGGGATTCTGAGACTCAACAGTGTATGATGCCTGTTAC
TAGCATTCACATGGAGAGCTCGCTGATCAGCCTCGACTGTGCCTTCT
AGTTGCCAGCCATCTGTTGTTTGCCCCTCCCCCGTGCCTTCCTTGAC
CCTGGAAGGTGCCACTCCCACTGTCCTTTCCTAATAAAATGAGGAAA
TTGCATCGCATTGTCTGAGTAGGTGTCATTCTATTCTGGGGGGTGGG
GTGGGGCAGGACAGCAAGGGGGAGGATTGGGAAGACAATAGCAGGCA
TGCTGGGGATGCGGTGGGCTCTATGGAAGCTTGAATTCAGCTGACGT
GCCTCGGACCGCTAGGAACCCCTAGTGATGGAGTTGGCCACTCCCTC
TCTGCGCGCTCGCTCGCTCACTGAGGCCGGGCGACCAAAGGTCGCCC
GACGCCCGGGCTTTGCCCGGGCGGCCTCAGTGAGCGAGCGAGCGCGC
AG
Exemplary pITR.CAG.miR2v2-155_EGFP sequence
(SEQ ID NO: 229)
CCTGCAGGCAGCTGCGCGCTCGCTCGCTCACTGAGGCCGCCCGGGCG
TCGGGCGACCTTTGGTCGCCCGGCCTCAGTGAGCGAGCGAGCGCGCA
GAGAGGGAGTGGCCAACTCCATCACTAGGGGTTCCTGCGGCCGCACG
CGTGACATTGATTATTGACTAGTTATTAATAGTAATCAATTACGGGG
TCATTAGTTCATAGCCCATATATGGAGTTCCGCGTTACATAACTTAC
GGTAAATGGCCCGCCTGGCTGACCGCCCAACGACCCCCGCCCATTGA
CGTCAATAATGACGTATGTTCCCATAGTAACGCCAATAGGGACTTTC
CATTGACGTCAATGGGTGGACTATTTACGGTAAACTGCCCACTTGGC
AGTACATCAAGTGTATCATATGCCAAGTACGCCCCCTATTGACGTCA
ATGACGGTAAATGGCCCGCCTGGCATTATGCCCAGTACATGACCTTA
TGGGACTTTCCTACTTGGCAGTACATCTACGTATTAGTCATCGCTAT
TACCATGGGTCGAGGTGAGCCCCACGTTCTGCTTCACTCTCCCCATC
TCCCCCCCCTCCCCACCCCCAATTTTGTATTTATTTATTTTTTAATT
ATTTTGTGCAGCGATGGGGGCGGGGGGGGGGGGGGCGCGCGCCAGGC
GGGGCGGGGCGGGGCGAGGGGCGGGGCGGGGCGAGGCGGAGAGGTGC
GGCGGCAGCCAATCAGAGCGGCGCGCTCCGAAAGTTTCCTTTTATGG
CGAGGCGGCGGCGGCGGCGGCCCTATAAAAAGCGAAGCGCGCGGCGG
GCGGGAGTCGCTGCGTTGCCTTCGCCCCGTGCCCCGCTCCGCGCCGC
CTCGCGCCGCCCGCCCCGGCTCTGACTGACCGCGTTACTCCCACAGG
TGAGCGGGCGGGACGGCCCTTCTCCTCCGGGCTGTAATTAGCGCTTG
GTTTAATGACGGCTCGTTTCTTTTCTGTGGCTGCGTGAAAGCCTTAA
AGGGCTCCGGGAGGGCCCTTTGTGCGGGGGGGAGCGGCTCGGGGGGT
GCGTGCGTGTGTGTGTGCGTGGGGAGCGCCGCGTGCGGCCCGCGCTG
CCCGGCGGCTGTGAGCGCTGCGGGCGCGGCGCGGGGCTTTGTGCGCT
CCGCGTGTGCGCGAGGGGAGCGCGGCCGGGGGCGGTGCCCCGCGGTG
CGGGGGGGCTGCGAGGGGAACAAAGGCTGCGTGCGGGGTGTGTGCGT
GGGGGGGTGAGCAGGGGGTGTGGGCGCGGCGGTCGGGCTGTAACCCC
CCCCTGCACCCCCCTCCCCGAGTTGCTGAGCACGGCCCGGCTTCGGG
TGCGGGGCTCCGTGCGGGGCGTGGCGCGGGGCTCGCCGTGCCGGGCG
GGGGGTGGCGGCAGGTGGGGGTGCCGGGCGGGGCGGGGCCGCCTCGG
GCCGGGGAGGGCTCGGGGGAGGGGCGCGGCGGCCCCCGGAGCGCCGG
CGTCCTCTGAGTGCTGAAGGCTTGCTGTAGGCTGTATGCTGTTGAGT
CTCAGAGATGCCCGGGTTTTTGCCTCCAACTGACTCGGGATTCTGAG
ATTCAACAGTGTATGATGCCTGTTACTAGCATTCACATGGATCTAGA
GCCTCTGCTAACCATGTTCATGCCTTCTTCTTTTTCCTACAGCTCCT
GGGCAACGTGCTGGTTATTGTGACCGGTCGCTAGCCACCATGGTGAG
CAAGGGCGAGGAGCTGTTCACCGGGGTGGTGCCCATCCTGGTCGAGC
TGGACGGCGACGTAAACGGCCACAAGTTCAGCGTGTCCGGCGAGGGC
GAGGGCGATGCCACCTACGGCAAGCTGACCCTGAAGTTCATCTGCAC
CACCGGCAAGCTGCCCGTGCCCTGGCCCACCCTCGTGACCACCCTGA
CCTACGGCGTGCAGTGCTTCAGCCGCTACCCCGACCACATGAAGCAG
CACGACTTCTTCAAGTCCGCCATGCCCGAAGGCTACGTCCAGGAGCG
CACCATCTTCTTCAAGGACGACGGCAACTACAAGACCCGCGCCGAGG
TGAAGTTCGAGGGCGACACCCTGGTGAACCGCATCGAGCTGAAGGGC
ATCGACTTCAAGGAGGACGGCAACATCCTGGGGCACAAGCTGGAGTA
CAACTACAACAGCCACAACGTCTATATCATGGCCGACAAGCAGAAGA
ACGGCATCAAGGTGAACTTCAAGATCCGCCACAACATCGAGGACGGC
AGCGTGCAGCTCGCCGACCACTACCAGCAGAACACCCCCATCGGCGA
CGGCCCCGTGCTGCTGCCCGACAACCACTACCTGAGCACCCAGTCCG
CCCTGAGCAAAGACCCCAACGAGAAGCGCGATCACATGGTCCTGCTG
GAGTTCGTGACCGCCGCCGGGATCACTCTCGGCATGGACGAGCTGTA
CAAGTAACCTGCAGGCGCCGGCGTCCTCTGAGTGCTGAAGGCTTGCT
GTAGGCTGTATGCTGTTGAGTCTCAGAGATGCCCGGGTTTTTGCCTC
CAACTGACTCGGGATTCTGAGATTCAACAGTGTATGATGCCTGTTAC
TAGCATTCACATGGAGAGCTCGCTGATCAGCCTCGACTGTGCCTTCT
AGTTGCCAGCCATCTGTTGTTTGCCCCTCCCCCGTGCCTTCCTTGAC
CCTGGAAGGTGCCACTCCCACTGTCCTTTCCTAATAAAATGAGGAAA
TTGCATCGCATTGTCTGAGTAGGTGTCATTCTATTCTGGGGGGTGGG
GTGGGGCAGGACAGCAAGGGGGAGGATTGGGAAGACAATAGCAGGCA
TGCTGGGGATGCGGTGGGCTCTATGGAAGCTTGAATTCAGCTGACGT
GCCTCGGACCGCTAGGAACCCCTAGTGATGGAGTTGGCCACTCCCTC
TCTGCGCGCTCGCTCGCTCACTGAGGCCGGGCGACCAAAGGTCGCCC
GACGCCCGGGCTTTGCCCGGGCGGCCTCAGTGAGCGAGCGAGCGCGC
AG
Exemplary pITR.CAG.miR2v3-155_EGFP sequence
(SEQ ID NO: 230)
CCTGCAGGCAGCTGCGCGCTCGCTCGCTCACTGAGGCCGCCCGGGCG
TCGGGCGACCTTTGGTCGCCCGGCCTCAGTGAGCGAGCGAGCGCGCA
GAGAGGGAGTGGCCAACTCCATCACTAGGGGTTCCTGCGGCCGCACG
CGTGACATTGATTATTGACTAGTTATTAATAGTAATCAATTACGGGG
TCATTAGTTCATAGCCCATATATGGAGTTCCGCGTTACATAACTTAC
GGTAAATGGCCCGCCTGGCTGACCGCCCAACGACCCCCGCCCATTGA
CGTCAATAATGACGTATGTTCCCATAGTAACGCCAATAGGGACTTTC
CATTGACGTCAATGGGTGGACTATTTACGGTAAACTGCCCACTTGGC
AGTACATCAAGTGTATCATATGCCAAGTACGCCCCCTATTGACGTCA
ATGACGGTAAATGGCCCGCCTGGCATTATGCCCAGTACATGACCTTA
TGGGACTTTCCTACTTGGCAGTACATCTACGTATTAGTCATCGCTAT
TACCATGGGTCGAGGTGAGCCCCACGTTCTGCTTCACTCTCCCCATC
TCCCCCCCCTCCCCACCCCCAATTTTGTATTTATTTATTTTTTAATT
ATTTTGTGCAGCGATGGGGGCGGGGGGGGGGGGGGCGCGCGCCAGGC
GGGGCGGGGCGGGGCGAGGGGCGGGGCGGGGCGAGGCGGAGAGGTGC
GGCGGCAGCCAATCAGAGCGGCGCGCTCCGAAAGTTTCCTTTTATGG
CGAGGCGGCGGCGGCGGCGGCCCTATAAAAAGCGAAGCGCGCGGCGG
GCGGGAGTCGCTGCGTTGCCTTCGCCCCGTGCCCCGCTCCGCGCCGC
CTCGCGCCGCCCGCCCCGGCTCTGACTGACCGCGTTACTCCCACAGG
TGAGCGGGCGGGACGGCCCTTCTCCTCCGGGCTGTAATTAGCGCTTG
GTTTAATGACGGCTCGTTTCTTTTCTGTGGCTGCGTGAAAGCCTTAA
AGGGCTCCGGGAGGGCCCTTTGTGCGGGGGGGAGCGGCTCGGGGGGT
GCGTGCGTGTGTGTGTGCGTGGGGAGCGCCGCGTGCGGCCCGCGCTG
CCCGGCGGCTGTGAGCGCTGCGGGCGCGGCGCGGGGCTTTGTGCGCT
CCGCGTGTGCGCGAGGGGAGCGCGGCCGGGGGCGGTGCCCCGCGGTG
CGGGGGGGCTGCGAGGGGAACAAAGGCTGCGTGCGGGGTGTGTGCGT
GGGGGGGTGAGCAGGGGGTGTGGGCGCGGCGGTCGGGCTGTAACCCC
CCCCTGCACCCCCCTCCCCGAGTTGCTGAGCACGGCCCGGCTTCGGG
TGCGGGGCTCCGTGCGGGGCGTGGCGCGGGGCTCGCCGTGCCGGGCG
GGGGGTGGCGGCAGGTGGGGGTGCCGGGCGGGGCGGGGCCGCCTCGG
GCCGGGGAGGGCTCGGGGGAGGGGCGCGGCGGCCCCCGGAGCGCCGG
CGTCCTCTGAGTGCTGAAGGCTTGCTGTAGGCTGTATGCTGTTGAGT
CTCAGAGATGCCCGGGTTTTTGCCTCCAACTGACTCGGGATCCTGAG
ATTCAACAGTGTATGATGCCTGTTACTAGCATTCACATGGATCTAGA
GCCTCTGCTAACCATGTTCATGCCTTCTTCTTTTTCCTACAGCTCCT
GGGCAACGTGCTGGTTATTGTGACCGGTCGCTAGCCACCATGGTGAG
CAAGGGCGAGGAGCTGTTCACCGGGGTGGTGCCCATCCTGGTCGAGC
TGGACGGCGACGTAAACGGCCACAAGTTCAGCGTGTCCGGCGAGGGC
GAGGGCGATGCCACCTACGGCAAGCTGACCCTGAAGTTCATCTGCAC
CACCGGCAAGCTGCCCGTGCCCTGGCCCACCCTCGTGACCACCCTGA
CCTACGGCGTGCAGTGCTTCAGCCGCTACCCCGACCACATGAAGCAG
CACGACTTCTTCAAGTCCGCCATGCCCGAAGGCTACGTCCAGGAGCG
CACCATCTTCTTCAAGGACGACGGCAACTACAAGACCCGCGCCGAGG
TGAAGTTCGAGGGCGACACCCTGGTGAACCGCATCGAGCTGAAGGGC
ATCGACTTCAAGGAGGACGGCAACATCCTGGGGCACAAGCTGGAGTA
CAACTACAACAGCCACAACGTCTATATCATGGCCGACAAGCAGAAGA
ACGGCATCAAGGTGAACTTCAAGATCCGCCACAACATCGAGGACGGC
AGCGTGCAGCTCGCCGACCACTACCAGCAGAACACCCCCATCGGCGA
CGGCCCCGTGCTGCTGCCCGACAACCACTACCTGAGCACCCAGTCCG
CCCTGAGCAAAGACCCCAACGAGAAGCGCGATCACATGGTCCTGCTG
GAGTTCGTGACCGCCGCCGGGATCACTCTCGGCATGGACGAGCTGTA
CAAGTAACCTGCAGGCGCCGGCGTCCTCTGAGTGCTGAAGGCTTGCT
GTAGGCTGTATGCTGTTGAGTCTCAGAGATGCCCGGGTTTTTGCCTC
CAACTGACTCGGGATCCTGAGATTCAACAGTGTATGATGCCTGTTAC
TAGCATTCACATGGAGAGCTCGCTGATCAGCCTCGACTGTGCCTTCT
AGTTGCCAGCCATCTGTTGTTTGCCCCTCCCCCGTGCCTTCCTTGAC
CCTGGAAGGTGCCACTCCCACTGTCCTTTCCTAATAAAATGAGGAAA
TTGCATCGCATTGTCTGAGTAGGTGTCATTCTATTCTGGGGGGTGGG
GTGGGGCAGGACAGCAAGGGGGAGGATTGGGAAGACAATAGCAGGCA
TGCTGGGGATGCGGTGGGCTCTATGGAAGCTTGAATTCAGCTGACGT
GCCTCGGACCGCTAGGAACCCCTAGTGATGGAGTTGGCCACTCCCTC
TCTGCGCGCTCGCTCGCTCACTGAGGCCGGGCGACCAAAGGTCGCCC
GACGCCCGGGCTTTGCCCGGGCGGCCTCAGTGAGCGAGCGAGCGCGC
AG
Exemplary pITR.CAG.miR2v4-155_EGFP sequence
(SEQ ID NO: 231)
CCTGCAGGCAGCTGCGCGCTCGCTCGCTCACTGAGGCCGCCCGGGCG
TCGGGCGACCTTTGGTCGCCCGGCCTCAGTGAGCGAGCGAGCGCGCA
GAGAGGGAGTGGCCAACTCCATCACTAGGGGTTCCTGCGGCCGCACG
CGTGACATTGATTATTGACTAGTTATTAATAGTAATCAATTACGGGG
TCATTAGTTCATAGCCCATATATGGAGTTCCGCGTTACATAACTTAC
GGTAAATGGCCCGCCTGGCTGACCGCCCAACGACCCCCGCCCATTGA
CGTCAATAATGACGTATGTTCCCATAGTAACGCCAATAGGGACTTTC
CATTGACGTCAATGGGTGGACTATTTACGGTAAACTGCCCACTTGGC
AGTACATCAAGTGTATCATATGCCAAGTACGCCCCCTATTGACGTCA
ATGACGGTAAATGGCCCGCCTGGCATTATGCCCAGTACATGACCTTA
TGGGACTTTCCTACTTGGCAGTACATCTACGTATTAGTCATCGCTAT
TACCATGGGTCGAGGTGAGCCCCACGTTCTGCTTCACTCTCCCCATC
TCCCCCCCCTCCCCACCCCCAATTTTGTATTTATTTATTTTTTAATT
ATTTTGTGCAGCGATGGGGGCGGGGGGGGGGGGGGCGCGCGCCAGGC
GGGGCGGGGCGGGGCGAGGGGCGGGGCGGGGCGAGGCGGAGAGGTGC
GGCGGCAGCCAATCAGAGCGGCGCGCTCCGAAAGTTTCCTTTTATGG
CGAGGCGGCGGCGGCGGCGGCCCTATAAAAAGCGAAGCGCGCGGCGG
GCGGGAGTCGCTGCGTTGCCTTCGCCCCGTGCCCCGCTCCGCGCCGC
CTCGCGCCGCCCGCCCCGGCTCTGACTGACCGCGTTACTCCCACAGG
TGAGCGGGCGGGACGGCCCTTCTCCTCCGGGCTGTAATTAGCGCTTG
GTTTAATGACGGCTCGTTTCTTTTCTGTGGCTGCGTGAAAGCCTTAA
AGGGCTCCGGGAGGGCCCTTTGTGCGGGGGGGAGCGGCTCGGGGGGT
GCGTGCGTGTGTGTGTGCGTGGGGAGCGCCGCGTGCGGCCCGCGCTG
CCCGGCGGCTGTGAGCGCTGCGGGCGCGGCGCGGGGCTTTGTGCGCT
CCGCGTGTGCGCGAGGGGAGCGCGGCCGGGGGCGGTGCCCCGCGGTG
CGGGGGGGCTGCGAGGGGAACAAAGGCTGCGTGCGGGGTGTGTGCGT
GGGGGGGTGAGCAGGGGGTGTGGGCGCGGCGGTCGGGCTGTAACCCC
CCCCTGCACCCCCCTCCCCGAGTTGCTGAGCACGGCCCGGCTTCGGG
TGCGGGGCTCCGTGCGGGGCGTGGCGCGGGGCTCGCCGTGCCGGGCG
GGGGGTGGCGGCAGGTGGGGGTGCCGGGCGGGGCGGGGCCGCCTCGG
GCCGGGGAGGGCTCGGGGGAGGGGCGCGGCGGCCCCCGGAGCGCCGG
CGTCCTCTGAGTGCTGAAGGCTTGCTGTAGGCTGTATGCTGTTGAGT
CTCAGAGATGCCCGGAAGTTTGCCTCCAACTCTTTCGGGATCCTGAG
ATTCAACAGTGTATGATGCCTGTTACTAGCATTCACATGGATCTAGA
GCCTCTGCTAACCATGTTCATGCCTTCTTCTTTTTCCTACAGCTCCT
GGGCAACGTGCTGGTTATTGTGACCGGTCGCTAGCCACCATGGTGAG
CAAGGGCGAGGAGCTGTTCACCGGGGTGGTGCCCATCCTGGTCGAGC
TGGACGGCGACGTAAACGGCCACAAGTTCAGCGTGTCCGGCGAGGGC
GAGGGCGATGCCACCTACGGCAAGCTGACCCTGAAGTTCATCTGCAC
CACCGGCAAGCTGCCCGTGCCCTGGCCCACCCTCGTGACCACCCTGA
CCTACGGCGTGCAGTGCTTCAGCCGCTACCCCGACCACATGAAGCAG
CACGACTTCTTCAAGTCCGCCATGCCCGAAGGCTACGTCCAGGAGCG
CACCATCTTCTTCAAGGACGACGGCAACTACAAGACCCGCGCCGAGG
TGAAGTTCGAGGGCGACACCCTGGTGAACCGCATCGAGCTGAAGGGC
ATCGACTTCAAGGAGGACGGCAACATCCTGGGGCACAAGCTGGAGTA
CAACTACAACAGCCACAACGTCTATATCATGGCCGACAAGCAGAAGA
ACGGCATCAAGGTGAACTTCAAGATCCGCCACAACATCGAGGACGGC
AGCGTGCAGCTCGCCGACCACTACCAGCAGAACACCCCCATCGGCGA
CGGCCCCGTGCTGCTGCCCGACAACCACTACCTGAGCACCCAGTCCG
CCCTGAGCAAAGACCCCAACGAGAAGCGCGATCACATGGTCCTGCTG
GAGTTCGTGACCGCCGCCGGGATCACTCTCGGCATGGACGAGCTGTA
CAAGTAACCTGCAGGCGCCGGCGTCCTCTGAGTGCTGAAGGCTTGCT
GTAGGCTGTATGCTGTTGAGTCTCAGAGATGCCCGGAAGTTTGCCTC
CAACTCTTTCGGGATCCTGAGATTCAACAGTGTATGATGCCTGTTAC
TAGCATTCACATGGAGAGCTCGCTGATCAGCCTCGACTGTGCCTTCT
AGTTGCCAGCCATCTGTTGTTTGCCCCTCCCCCGTGCCTTCCTTGAC
CCTGGAAGGTGCCACTCCCACTGTCCTTTCCTAATAAAATGAGGAAA
TTGCATCGCATTGTCTGAGTAGGTGTCATTCTATTCTGGGGGGTGGG
GTGGGGCAGGACAGCAAGGGGGAGGATTGGGAAGACAATAGCAGGCA
TGCTGGGGATGCGGTGGGCTCTATGGAAGCTTGAATTCAGCTGACGT
GCCTCGGACCGCTAGGAACCCCTAGTGATGGAGTTGGCCACTCCCTC
TCTGCGCGCTCGCTCGCTCACTGAGGCCGGGCGACCAAAGGTCGCCC
GACGCCCGGGCTTTGCCCGGGCGGCCTCAGTGAGCGAGCGAGCGCGC
AG
Exemplary pITR.CAG.miR4-155_EGFP sequence
(SEQ ID NO: 232)
CCTGCAGGCAGCTGCGCGCTCGCTCGCTCACTGAGGCCGCCCGGGCG
TCGGGCGACCTTTGGTCGCCCGGCCTCAGTGAGCGAGCGAGCGCGCA
GAGAGGGAGTGGCCAACTCCATCACTAGGGGTTCCTGCGGCCGCACG
CGTGACATTGATTATTGACTAGTTATTAATAGTAATCAATTACGGGG
TCATTAGTTCATAGCCCATATATGGAGTTCCGCGTTACATAACTTAC
GGTAAATGGCCCGCCTGGCTGACCGCCCAACGACCCCCGCCCATTGA
CGTCAATAATGACGTATGTTCCCATAGTAACGCCAATAGGGACTTTC
CATTGACGTCAATGGGTGGACTATTTACGGTAAACTGCCCACTTGGC
AGTACATCAAGTGTATCATATGCCAAGTACGCCCCCTATTGACGTCA
ATGACGGTAAATGGCCCGCCTGGCATTATGCCCAGTACATGACCTTA
TGGGACTTTCCTACTTGGCAGTACATCTACGTATTAGTCATCGCTAT
TACCATGGGTCGAGGTGAGCCCCACGTTCTGCTTCACTCTCCCCATC
TCCCCCCCCTCCCCACCCCCAATTTTGTATTTATTTATTTTTTAATT
ATTTTGTGCAGCGATGGGGGCGGGGGGGGGGGGGGCGCGCGCCAGGC
GGGGCGGGGCGGGGCGAGGGGCGGGGCGGGGCGAGGCGGAGAGGTGC
GGCGGCAGCCAATCAGAGCGGCGCGCTCCGAAAGTTTCCTTTTATGG
CGAGGCGGCGGCGGCGGCGGCCCTATAAAAAGCGAAGCGCGCGGCGG
GCGGGAGTCGCTGCGTTGCCTTCGCCCCGTGCCCCGCTCCGCGCCGC
CTCGCGCCGCCCGCCCCGGCTCTGACTGACCGCGTTACTCCCACAGG
TGAGCGGGCGGGACGGCCCTTCTCCTCCGGGCTGTAATTAGCGCTTG
GTTTAATGACGGCTCGTTTCTTTTCTGTGGCTGCGTGAAAGCCTTAA
AGGGCTCCGGGAGGGCCCTTTGTGCGGGGGGGAGCGGCTCGGGGGGT
GCGTGCGTGTGTGTGTGCGTGGGGAGCGCCGCGTGCGGCCCGCGCTG
CCCGGCGGCTGTGAGCGCTGCGGGCGCGGCGCGGGGCTTTGTGCGCT
CCGCGTGTGCGCGAGGGGAGCGCGGCCGGGGGCGGTGCCCCGCGGTG
CGGGGGGGCTGCGAGGGGAACAAAGGCTGCGTGCGGGGTGTGTGCGT
GGGGGGGTGAGCAGGGGGTGTGGGCGCGGCGGTCGGGCTGTAACCCC
CCCCTGCACCCCCCTCCCCGAGTTGCTGAGCACGGCCCGGCTTCGGG
TGCGGGGCTCCGTGCGGGGCGTGGCGCGGGGCTCGCCGTGCCGGGCG
GGGGGTGGCGGCAGGTGGGGGTGCCGGGCGGGGCGGGGCCGCCTCGG
GCCGGGGAGGGCTCGGGGGAGGGGCGCGGCGGCCCCCGGAGCGCCGG
CGTCCTCTGAGTGCTGAAGGCTTGCTGTAGGCTGTATGCTGTTCTCG
AAGTGCTTCTGCCGGGTTTTTGCCTCCAACTGACTCGGCGAATACTT
CGAGAACAGTGTATGATGCCTGTTACTAGCATTCACATGGATCTAGA
GCCTCTGCTAACCATGTTCATGCCTTCTTCTTTTTCCTACAGCTCCT
GGGCAACGTGCTGGTTATTGTGACCGGTCGCTAGCCACCATGGTGAG
CAAGGGCGAGGAGCTGTTCACCGGGGTGGTGCCCATCCTGGTCGAGC
TGGACGGCGACGTAAACGGCCACAAGTTCAGCGTGTCCGGCGAGGGC
GAGGGCGATGCCACCTACGGCAAGCTGACCCTGAAGTTCATCTGCAC
CACCGGCAAGCTGCCCGTGCCCTGGCCCACCCTCGTGACCACCCTGA
CCTACGGCGTGCAGTGCTTCAGCCGCTACCCCGACCACATGAAGCAG
CACGACTTCTTCAAGTCCGCCATGCCCGAAGGCTACGTCCAGGAGCG
CACCATCTTCTTCAAGGACGACGGCAACTACAAGACCCGCGCCGAGG
TGAAGTTCGAGGGCGACACCCTGGTGAACCGCATCGAGCTGAAGGGC
ATCGACTTCAAGGAGGACGGCAACATCCTGGGGCACAAGCTGGAGTA
CAACTACAACAGCCACAACGTCTATATCATGGCCGACAAGCAGAAGA
ACGGCATCAAGGTGAACTTCAAGATCCGCCACAACATCGAGGACGGC
AGCGTGCAGCTCGCCGACCACTACCAGCAGAACACCCCCATCGGCGA
CGGCCCCGTGCTGCTGCCCGACAACCACTACCTGAGCACCCAGTCCG
CCCTGAGCAAAGACCCCAACGAGAAGCGCGATCACATGGTCCTGCTG
GAGTTCGTGACCGCCGCCGGGATCACTCTCGGCATGGACGAGCTGTA
CAAGTAACCTGCAGGCGCCGGCGTCCTCTGAGTGCTGAAGGCTTGCT
GTAGGCTGTATGCTGTTCTCGAAGTGCTTCTGCCGGGTTTTTGCCTC
CAACTGACTCGGCGAATACTTCGAGAACAGTGTATGATGCCTGTTAC
TAGCATTCACATGGAGAGCTCGCTGATCAGCCTCGACTGTGCCTTCT
AGTTGCCAGCCATCTGTTGTTTGCCCCTCCCCCGTGCCTTCCTTGAC
CCTGGAAGGTGCCACTCCCACTGTCCTTTCCTAATAAAATGAGGAAA
TTGCATCGCATTGTCTGAGTAGGTGTCATTCTATTCTGGGGGGTGGG
GTGGGGCAGGACAGCAAGGGGGAGGATTGGGAAGACAATAGCAGGCA
TGCTGGGGATGCGGTGGGCTCTATGGAAGCTTGAATTCAGCTGACGT
GCCTCGGACCGCTAGGAACCCCTAGTGATGGAGTTGGCCACTCCCTC
TCTGCGCGCTCGCTCGCTCACTGAGGCCGGGCGACCAAAGGTCGCCC
GACGCCCGGGCTTTGCCCGGGCGGCCTCAGTGAGCGAGCGAGCGCGC
AG
Exemplary pITR.CAG.miR5-155_EGFP sequence
(SEQ ID NO: 233)
CCTGCAGGCAGCTGCGCGCTCGCTCGCTCACTGAGGCCGCCCGGGCG
TCGGGCGACCTTTGGTCGCCCGGCCTCAGTGAGCGAGCGAGCGCGCA
GAGAGGGAGTGGCCAACTCCATCACTAGGGGTTCCTGCGGCCGCACG
CGTGACATTGATTATTGACTAGTTATTAATAGTAATCAATTACGGGG
TCATTAGTTCATAGCCCATATATGGAGTTCCGCGTTACATAACTTAC
GGTAAATGGCCCGCCTGGCTGACCGCCCAACGACCCCCGCCCATTGA
CGTCAATAATGACGTATGTTCCCATAGTAACGCCAATAGGGACTTTC
CATTGACGTCAATGGGTGGACTATTTACGGTAAACTGCCCACTTGGC
AGTACATCAAGTGTATCATATGCCAAGTACGCCCCCTATTGACGTCA
ATGACGGTAAATGGCCCGCCTGGCATTATGCCCAGTACATGACCTTA
TGGGACTTTCCTACTTGGCAGTACATCTACGTATTAGTCATCGCTAT
TACCATGGGTCGAGGTGAGCCCCACGTTCTGCTTCACTCTCCCCATC
TCCCCCCCCTCCCCACCCCCAATTTTGTATTTATTTATTTTTTAATT
ATTTTGTGCAGCGATGGGGGCGGGGGGGGGGGGGGCGCGCGCCAGGC
GGGGCGGGGCGGGGCGAGGGGCGGGGCGGGGCGAGGCGGAGAGGTGC
GGCGGCAGCCAATCAGAGCGGCGCGCTCCGAAAGTTTCCTTTTATGG
CGAGGCGGCGGCGGCGGCGGCCCTATAAAAAGCGAAGCGCGCGGCGG
GCGGGAGTCGCTGCGTTGCCTTCGCCCCGTGCCCCGCTCCGCGCCGC
CTCGCGCCGCCCGCCCCGGCTCTGACTGACCGCGTTACTCCCACAGG
TGAGCGGGCGGGACGGCCCTTCTCCTCCGGGCTGTAATTAGCGCTTG
GTTTAATGACGGCTCGTTTCTTTTCTGTGGCTGCGTGAAAGCCTTAA
AGGGCTCCGGGAGGGCCCTTTGTGCGGGGGGGAGCGGCTCGGGGGGT
GCGTGCGTGTGTGTGTGCGTGGGGAGCGCCGCGTGCGGCCCGCGCTG
CCCGGCGGCTGTGAGCGCTGCGGGCGCGGCGCGGGGCTTTGTGCGCT
CCGCGTGTGCGCGAGGGGAGCGCGGCCGGGGGCGGTGCCCCGCGGTG
CGGGGGGGCTGCGAGGGGAACAAAGGCTGCGTGCGGGGTGTGTGCGT
GGGGGGGTGAGCAGGGGGTGTGGGCGCGGCGGTCGGGCTGTAACCCC
CCCCTGCACCCCCCTCCCCGAGTTGCTGAGCACGGCCCGGCTTCGGG
TGCGGGGCTCCGTGCGGGGCGTGGCGCGGGGCTCGCCGTGCCGGGCG
GGGGGTGGCGGCAGGTGGGGGTGCCGGGCGGGGCGGGGCCGCCTCGG
GCCGGGGAGGGCTCGGGGGAGGGGCGCGGCGGCCCCCGGAGCGCCGG
CGTCCTCTGAGTGCTGAAGGCTTGCTGTAGGCTGTATGCTGTTCTCC
ACCTTGACCACGCGTGTTTTTGCCTCCAACTGACATGTGGGTAAGGT
GGAGAACAGTGTATGATGCCTGTTACTAGCATTCACATGGATCTAGA
GCCTCTGCTAACCATGTTCATGCCTTCTTCTTTTTCCTACAGCTCCT
GGGCAACGTGCTGGTTATTGTGACCGGTCGCTAGCCACCATGGTGAG
CAAGGGCGAGGAGCTGTTCACCGGGGTGGTGCCCATCCTGGTCGAGC
TGGACGGCGACGTAAACGGCCACAAGTTCAGCGTGTCCGGCGAGGGC
GAGGGCGATGCCACCTACGGCAAGCTGACCCTGAAGTTCATCTGCAC
CACCGGCAAGCTGCCCGTGCCCTGGCCCACCCTCGTGACCACCCTGA
CCTACGGCGTGCAGTGCTTCAGCCGCTACCCCGACCACATGAAGCAG
CACGACTTCTTCAAGTCCGCCATGCCCGAAGGCTACGTCCAGGAGCG
CACCATCTTCTTCAAGGACGACGGCAACTACAAGACCCGCGCCGAGG
TGAAGTTCGAGGGCGACACCCTGGTGAACCGCATCGAGCTGAAGGGC
ATCGACTTCAAGGAGGACGGCAACATCCTGGGGCACAAGCTGGAGTA
CAACTACAACAGCCACAACGTCTATATCATGGCCGACAAGCAGAAGA
ACGGCATCAAGGTGAACTTCAAGATCCGCCACAACATCGAGGACGGC
AGCGTGCAGCTCGCCGACCACTACCAGCAGAACACCCCCATCGGCGA
CGGCCCCGTGCTGCTGCCCGACAACCACTACCTGAGCACCCAGTCCG
CCCTGAGCAAAGACCCCAACGAGAAGCGCGATCACATGGTCCTGCTG
GAGTTCGTGACCGCCGCCGGGATCACTCTCGGCATGGACGAGCTGTA
CAAGTAACCTGCAGGCGCCGGCGTCCTCTGAGTGCTGAAGGCTTGCT
GTAGGCTGTATGCTGTTCTCCACCTTGACCACGCGTGTTTTTGCCTC
CAACTGACATGTGGGTAAGGTGGAGAACAGTGTATGATGCCTGTTAC
TAGCATTCACATGGAGAGCTCGCTGATCAGCCTCGACTGTGCCTTCT
AGTTGCCAGCCATCTGTTGTTTGCCCCTCCCCCGTGCCTTCCTTGAC
CCTGGAAGGTGCCACTCCCACTGTCCTTTCCTAATAAAATGAGGAAA
TTGCATCGCATTGTCTGAGTAGGTGTCATTCTATTCTGGGGGGTGGG
GTGGGGCAGGACAGCAAGGGGGAGGATTGGGAAGACAATAGCAGGCA
TGCTGGGGATGCGGTGGGCTCTATGGAAGCTTGAATTCAGCTGACGT
GCCTCGGACCGCTAGGAACCCCTAGTGATGGAGTTGGCCACTCCCTC
TCTGCGCGCTCGCTCGCTCACTGAGGCCGGGCGACCAAAGGTCGCCC
GACGCCCGGGCTTTGCCCGGGCGGCCTCAGTGAGCGAGCGAGCGCGC
AG
Exemplary pITR.CAG.miR6-155_EGFP sequence
(SEQ ID NO: 234)
CCTGCAGGCAGCTGCGCGCTCGCTCGCTCACTGAGGCCGCCCGGGCG
TCGGGCGACCTTTGGTCGCCCGGCCTCAGTGAGCGAGCGAGCGCGCA
GAGAGGGAGTGGCCAACTCCATCACTAGGGGTTCCTGCGGCCGCACG
CGTGACATTGATTATTGACTAGTTATTAATAGTAATCAATTACGGGG
TCATTAGTTCATAGCCCATATATGGAGTTCCGCGTTACATAACTTAC
GGTAAATGGCCCGCCTGGCTGACCGCCCAACGACCCCCGCCCATTGA
CGTCAATAATGACGTATGTTCCCATAGTAACGCCAATAGGGACTTTC
CATTGACGTCAATGGGTGGACTATTTACGGTAAACTGCCCACTTGGC
AGTACATCAAGTGTATCATATGCCAAGTACGCCCCCTATTGACGTCA
ATGACGGTAAATGGCCCGCCTGGCATTATGCCCAGTACATGACCTTA
TGGGACTTTCCTACTTGGCAGTACATCTACGTATTAGTCATCGCTAT
TACCATGGGTCGAGGTGAGCCCCACGTTCTGCTTCACTCTCCCCATC
TCCCCCCCCTCCCCACCCCCAATTTTGTATTTATTTATTTTTTAATT
ATTTTGTGCAGCGATGGGGGCGGGGGGGGGGGGGGCGCGCGCCAGGC
GGGGCGGGGCGGGGCGAGGGGCGGGGCGGGGCGAGGCGGAGAGGTGC
GGCGGCAGCCAATCAGAGCGGCGCGCTCCGAAAGTTTCCTTTTATGG
CGAGGCGGCGGCGGCGGCGGCCCTATAAAAAGCGAAGCGCGCGGCGG
GCGGGAGTCGCTGCGTTGCCTTCGCCCCGTGCCCCGCTCCGCGCCGC
CTCGCGCCGCCCGCCCCGGCTCTGACTGACCGCGTTACTCCCACAGG
TGAGCGGGCGGGACGGCCCTTCTCCTCCGGGCTGTAATTAGCGCTTG
GTTTAATGACGGCTCGTTTCTTTTCTGTGGCTGCGTGAAAGCCTTAA
AGGGCTCCGGGAGGGCCCTTTGTGCGGGGGGGAGCGGCTCGGGGGGT
GCGTGCGTGTGTGTGTGCGTGGGGAGCGCCGCGTGCGGCCCGCGCTG
CCCGGCGGCTGTGAGCGCTGCGGGCGCGGCGCGGGGCTTTGTGCGCT
CCGCGTGTGCGCGAGGGGAGCGCGGCCGGGGGCGGTGCCCCGCGGTG
CGGGGGGGCTGCGAGGGGAACAAAGGCTGCGTGCGGGGTGTGTGCGT
GGGGGGGTGAGCAGGGGGTGTGGGCGCGGCGGTCGGGCTGTAACCCC
CCCCTGCACCCCCCTCCCCGAGTTGCTGAGCACGGCCCGGCTTCGGG
TGCGGGGCTCCGTGCGGGGCGTGGCGCGGGGCTCGCCGTGCCGGGCG
GGGGGTGGCGGCAGGTGGGGGTGCCGGGCGGGGCGGGGCCGCCTCGG
GCCGGGGAGGGCTCGGGGGAGGGGCGCGGCGGCCCCCGGAGCGCCGG
CGTCCTCTGAGTGCTGAAGGCTTGCTGTAGGCTGTATGCTGATACTG
TCATAGTAGTACCAGGTTTTTGCCTCCAACTGACCTGGTCTATATGA
CAGTATCAGTGTATGATGCCTGTTACTAGCATTCACATGGATCTAGA
GCCTCTGCTAACCATGTTCATGCCTTCTTCTTTTTCCTACAGCTCCT
GGGCAACGTGCTGGTTATTGTGACCGGTCGCTAGCCACCATGGTGAG
CAAGGGCGAGGAGCTGTTCACCGGGGTGGTGCCCATCCTGGTCGAGC
TGGACGGCGACGTAAACGGCCACAAGTTCAGCGTGTCCGGCGAGGGC
GAGGGCGATGCCACCTACGGCAAGCTGACCCTGAAGTTCATCTGCAC
CACCGGCAAGCTGCCCGTGCCCTGGCCCACCCTCGTGACCACCCTGA
CCTACGGCGTGCAGTGCTTCAGCCGCTACCCCGACCACATGAAGCAG
CACGACTTCTTCAAGTCCGCCATGCCCGAAGGCTACGTCCAGGAGCG
CACCATCTTCTTCAAGGACGACGGCAACTACAAGACCCGCGCCGAGG
TGAAGTTCGAGGGCGACACCCTGGTGAACCGCATCGAGCTGAAGGGC
ATCGACTTCAAGGAGGACGGCAACATCCTGGGGCACAAGCTGGAGTA
CAACTACAACAGCCACAACGTCTATATCATGGCCGACAAGCAGAAGA
ACGGCATCAAGGTGAACTTCAAGATCCGCCACAACATCGAGGACGGC
AGCGTGCAGCTCGCCGACCACTACCAGCAGAACACCCCCATCGGCGA
CGGCCCCGTGCTGCTGCCCGACAACCACTACCTGAGCACCCAGTCCG
CCCTGAGCAAAGACCCCAACGAGAAGCGCGATCACATGGTCCTGCTG
GAGTTCGTGACCGCCGCCGGGATCACTCTCGGCATGGACGAGCTGTA
CAAGTAACCTGCAGGCGCCGGCGTCCTCTGAGTGCTGAAGGCTTGCT
GTAGGCTGTATGCTGATACTGTCATAGTAGTACCAGGTTTTTGCCTC
CAACTGACCTGGTCTATATGACAGTATCAGTGTATGATGCCTGTTAC
TAGCATTCACATGGAGAGCTCGCTGATCAGCCTCGACTGTGCCTTCT
AGTTGCCAGCCATCTGTTGTTTGCCCCTCCCCCGTGCCTTCCTTGAC
CCTGGAAGGTGCCACTCCCACTGTCCTTTCCTAATAAAATGAGGAAA
TTGCATCGCATTGTCTGAGTAGGTGTCATTCTATTCTGGGGGGTGGG
GTGGGGCAGGACAGCAAGGGGGAGGATTGGGAAGACAATAGCAGGCA
TGCTGGGGATGCGGTGGGCTCTATGGAAGCTTGAATTCAGCTGACGT
GCCTCGGACCGCTAGGAACCCCTAGTGATGGAGTTGGCCACTCCCTC
TCTGCGCGCTCGCTCGCTCACTGAGGCCGGGCGACCAAAGGTCGCCC
GACGCCCGGGCTTTGCCCGGGCGGCCTCAGTGAGCGAGCGAGCGCGC
AG
Exemplary pITR.CAG.miR7-155_EGFP sequence
(SEQ ID NO: 235)
CCTGCAGGCAGCTGCGCGCTCGCTCGCTCACTGAGGCCGCCCGGGCG
TCGGGCGACCTTTGGTCGCCCGGCCTCAGTGAGCGAGCGAGCGCGCA
GAGAGGGAGTGGCCAACTCCATCACTAGGGGTTCCTGCGGCCGCACG
CGTGACATTGATTATTGACTAGTTATTAATAGTAATCAATTACGGGG
TCATTAGTTCATAGCCCATATATGGAGTTCCGCGTTACATAACTTAC
GGTAAATGGCCCGCCTGGCTGACCGCCCAACGACCCCCGCCCATTGA
CGTCAATAATGACGTATGTTCCCATAGTAACGCCAATAGGGACTTTC
CATTGACGTCAATGGGTGGACTATTTACGGTAAACTGCCCACTTGGC
AGTACATCAAGTGTATCATATGCCAAGTACGCCCCCTATTGACGTCA
ATGACGGTAAATGGCCCGCCTGGCATTATGCCCAGTACATGACCTTA
TGGGACTTTCCTACTTGGCAGTACATCTACGTATTAGTCATCGCTAT
TACCATGGGTCGAGGTGAGCCCCACGTTCTGCTTCACTCTCCCCATC
TCCCCCCCCTCCCCACCCCCAATTTTGTATTTATTTATTTTTTAATT
ATTTTGTGCAGCGATGGGGGCGGGGGGGGGGGGGGCGCGCGCCAGGC
GGGGCGGGGCGGGGCGAGGGGCGGGGCGGGGCGAGGCGGAGAGGTGC
GGCGGCAGCCAATCAGAGCGGCGCGCTCCGAAAGTTTCCTTTTATGG
CGAGGCGGCGGCGGCGGCGGCCCTATAAAAAGCGAAGCGCGCGGCGG
GCGGGAGTCGCTGCGTTGCCTTCGCCCCGTGCCCCGCTCCGCGCCGC
CTCGCGCCGCCCGCCCCGGCTCTGACTGACCGCGTTACTCCCACAGG
TGAGCGGGCGGGACGGCCCTTCTCCTCCGGGCTGTAATTAGCGCTTG
GTTTAATGACGGCTCGTTTCTTTTCTGTGGCTGCGTGAAAGCCTTAA
AGGGCTCCGGGAGGGCCCTTTGTGCGGGGGGGAGCGGCTCGGGGGGT
GCGTGCGTGTGTGTGTGCGTGGGGAGCGCCGCGTGCGGCCCGCGCTG
CCCGGCGGCTGTGAGCGCTGCGGGCGCGGCGCGGGGCTTTGTGCGCT
CCGCGTGTGCGCGAGGGGAGCGCGGCCGGGGGCGGTGCCCCGCGGTG
CGGGGGGGCTGCGAGGGGAACAAAGGCTGCGTGCGGGGTGTGTGCGT
GGGGGGGTGAGCAGGGGGTGTGGGCGCGGCGGTCGGGCTGTAACCCC
CCCCTGCACCCCCCTCCCCGAGTTGCTGAGCACGGCCCGGCTTCGGG
TGCGGGGCTCCGTGCGGGGCGTGGCGCGGGGCTCGCCGTGCCGGGCG
GGGGGTGGCGGCAGGTGGGGGTGCCGGGCGGGGCGGGGCCGCCTCGG
GCCGGGGAGGGCTCGGGGGAGGGGCGCGGCGGCCCCCGGAGCGCCGG
CGTCCTCTGAGTGCTGAAGGCTTGCTGTAGGCTGTATGCTGTAGTCG
GAGGTGATGTCGGGGGTTTTTGCCTCCAACTGACTCCTGCATACCTT
CGATTACAGTGTATGATGCCTGTTACTAGCATTCACATGGATCTAGA
GCCTCTGCTAACCATGTTCATGCCTTCTTCTTTTTCCTACAGCTCCT
GGGCAACGTGCTGGTTATTGTGACCGGTCGCTAGCCACCATGGTGAG
CAAGGGCGAGGAGCTGTTCACCGGGGTGGTGCCCATCCTGGTCGAGC
TGGACGGCGACGTAAACGGCCACAAGTTCAGCGTGTCCGGCGAGGGC
GAGGGCGATGCCACCTACGGCAAGCTGACCCTGAAGTTCATCTGCAC
CACCGGCAAGCTGCCCGTGCCCTGGCCCACCCTCGTGACCACCCTGA
CCTACGGCGTGCAGTGCTTCAGCCGCTACCCCGACCACATGAAGCAG
CACGACTTCTTCAAGTCCGCCATGCCCGAAGGCTACGTCCAGGAGCG
CACCATCTTCTTCAAGGACGACGGCAACTACAAGACCCGCGCCGAGG
TGAAGTTCGAGGGCGACACCCTGGTGAACCGCATCGAGCTGAAGGGC
ATCGACTTCAAGGAGGACGGCAACATCCTGGGGCACAAGCTGGAGTA
CAACTACAACAGCCACAACGTCTATATCATGGCCGACAAGCAGAAGA
ACGGCATCAAGGTGAACTTCAAGATCCGCCACAACATCGAGGACGGC
AGCGTGCAGCTCGCCGACCACTACCAGCAGAACACCCCCATCGGCGA
CGGCCCCGTGCTGCTGCCCGACAACCACTACCTGAGCACCCAGTCCG
CCCTGAGCAAAGACCCCAACGAGAAGCGCGATCACATGGTCCTGCTG
GAGTTCGTGACCGCCGCCGGGATCACTCTCGGCATGGACGAGCTGTA
CAAGTAACCTGCAGGCGCCGGCGTCCTCTGAGTGCTGAAGGCTTGCT
GTAGGCTGTATGCTGTAGTCGGAGGTGATGTCGGGGGTTTTTGCCTC
CAACTGACTCCTGCATACCTTCGATTACAGTGTATGATGCCTGTTAC
TAGCATTCACATGGAGAGCTCGCTGATCAGCCTCGACTGTGCCTTCT
AGTTGCCAGCCATCTGTTGTTTGCCCCTCCCCCGTGCCTTCCTTGAC
CCTGGAAGGTGCCACTCCCACTGTCCTTTCCTAATAAAATGAGGAAA
TTGCATCGCATTGTCTGAGTAGGTGTCATTCTATTCTGGGGGGTGGG
GTGGGGCAGGACAGCAAGGGGGAGGATTGGGAAGACAATAGCAGGCA
TGCTGGGGATGCGGTGGGCTCTATGGAAGCTTGAATTCAGCTGACGT
GCCTCGGACCGCTAGGAACCCCTAGTGATGGAGTTGGCCACTCCCTC
TCTGCGCGCTCGCTCGCTCACTGAGGCCGGGCGACCAAAGGTCGCCC
GACGCCCGGGCTTTGCCCGGGCGGCCTCAGTGAGCGAGCGAGCGCGC
AG
Exemplary pITR.CAG.miR1-16_EGFP sequence
(SEQ ID NO: 236)
CCTGCAGGCAGCTGCGCGCTCGCTCGCTCACTGAGGCCGCCCGGGCG
TCGGGCGACCTTTGGTCGCCCGGCCTCAGTGAGCGAGCGAGCGCGCA
GAGAGGGAGTGGCCAACTCCATCACTAGGGGTTCCTGCGGCCGCACG
CGTGACATTGATTATTGACTAGTTATTAATAGTAATCAATTACGGGG
TCATTAGTTCATAGCCCATATATGGAGTTCCGCGTTACATAACTTAC
GGTAAATGGCCCGCCTGGCTGACCGCCCAACGACCCCCGCCCATTGA
CGTCAATAATGACGTATGTTCCCATAGTAACGCCAATAGGGACTTTC
CATTGACGTCAATGGGTGGACTATTTACGGTAAACTGCCCACTTGGC
AGTACATCAAGTGTATCATATGCCAAGTACGCCCCCTATTGACGTCA
ATGACGGTAAATGGCCCGCCTGGCATTATGCCCAGTACATGACCTTA
TGGGACTTTCCTACTTGGCAGTACATCTACGTATTAGTCATCGCTAT
TACCATGGGTCGAGGTGAGCCCCACGTTCTGCTTCACTCTCCCCATC
TCCCCCCCCTCCCCACCCCCAATTTTGTATTTATTTATTTTTTAATT
ATTTTGTGCAGCGATGGGGGCGGGGGGGGGGGGGGCGCGCGCCAGGC
GGGGCGGGGCGGGGCGAGGGGCGGGGCGGGGCGAGGCGGAGAGGTGC
GGCGGCAGCCAATCAGAGCGGCGCGCTCCGAAAGTTTCCTTTTATGG
CGAGGCGGCGGCGGCGGCGGCCCTATAAAAAGCGAAGCGCGCGGCGG
GCGGGAGTCGCTGCGTTGCCTTCGCCCCGTGCCCCGCTCCGCGCCGC
CTCGCGCCGCCCGCCCCGGCTCTGACTGACCGCGTTACTCCCACAGG
TGAGCGGGCGGGACGGCCCTTCTCCTCCGGGCTGTAATTAGCGCTTG
GTTTAATGACGGCTCGTTTCTTTTCTGTGGCTGCGTGAAAGCCTTAA
AGGGCTCCGGGAGGGCCCTTTGTGCGGGGGGGAGCGGCTCGGGGGGT
GCGTGCGTGTGTGTGTGCGTGGGGAGCGCCGCGTGCGGCCCGCGCTG
CCCGGCGGCTGTGAGCGCTGCGGGCGCGGCGCGGGGCTTTGTGCGCT
CCGCGTGTGCGCGAGGGGAGCGCGGCCGGGGGCGGTGCCCCGCGGTG
CGGGGGGGCTGCGAGGGGAACAAAGGCTGCGTGCGGGGTGTGTGCGT
GGGGGGGTGAGCAGGGGGTGTGGGCGCGGCGGTCGGGCTGTAACCCC
CCCCTGCACCCCCCTCCCCGAGTTGCTGAGCACGGCCCGGCTTCGGG
TGCGGGGCTCCGTGCGGGGCGTGGCGCGGGGCTCGCCGTGCCGGGCG
GGGGGTGGCGGCAGGTGGGGGTGCCGGGCGGGGCGGGGCCGCCTCGG
GCCGGGGAGGGCTCGGGGGAGGGGCGCGGCGGCCCCCGGAGCGCCGG
CGCATAGCTCTTATGATAGCAATGTCAGCAGTGCCTTTGAGAATCCT
GATGGAGCGGGTTAAGATTCTAAAATTATCACGCTCCATGAGTGATT
CTCAGAAGTAAGGTTGACCATACTCTACAGTTGTGTCTAGAGCCTCT
GCTAACCATGTTCATGCCTTCTTCTTTTTCCTACAGCTCCTGGGCAA
CGTGCTGGTTATTGTGACCGGTCGCTAGCCACCATGGTGAGCAAGGG
CGAGGAGCTGTTCACCGGGGTGGTGCCCATCCTGGTCGAGCTGGACG
GCGACGTAAACGGCCACAAGTTCAGCGTGTCCGGCGAGGGCGAGGGC
GATGCCACCTACGGCAAGCTGACCCTGAAGTTCATCTGCACCACCGG
CAAGCTGCCCGTGCCCTGGCCCACCCTCGTGACCACCCTGACCTACG
GCGTGCAGTGCTTCAGCCGCTACCCCGACCACATGAAGCAGCACGAC
TTCTTCAAGTCCGCCATGCCCGAAGGCTACGTCCAGGAGCGCACCAT
CTTCTTCAAGGACGACGGCAACTACAAGACCCGCGCCGAGGTGAAGT
TCGAGGGCGACACCCTGGTGAACCGCATCGAGCTGAAGGGCATCGAC
TTCAAGGAGGACGGCAACATCCTGGGGCACAAGCTGGAGTACAACTA
CAACAGCCACAACGTCTATATCATGGCCGACAAGCAGAAGAACGGCA
TCAAGGTGAACTTCAAGATCCGCCACAACATCGAGGACGGCAGCGTG
CAGCTCGCCGACCACTACCAGCAGAACACCCCCATCGGCGACGGCCC
CGTGCTGCTGCCCGACAACCACTACCTGAGCACCCAGTCCGCCCTGA
GCAAAGACCCCAACGAGAAGCGCGATCACATGGTCCTGCTGGAGTTC
GTGACCGCCGCCGGGATCACTCTCGGCATGGACGAGCTGTACAAGTA
ACCTGCAGGCGCCGGCGCATAGCTCTTATGATAGCAATGTCAGCAGT
GCCTTTGAGAATCCTGATGGAGCGGGTTAAGATTCTAAAATTATCAC
GCTCCATGAGTGATTCTCAGAAGTAAGGTTGACCATACTCTACAGTT
GTGGAGCTCGCTGATCAGCCTCGACTGTGCCTTCTAGTTGCCAGCCA
TCTGTTGTTTGCCCCTCCCCCGTGCCTTCCTTGACCCTGGAAGGTGC
CACTCCCACTGTCCTTTCCTAATAAAATGAGGAAATTGCATCGCATT
GTCTGAGTAGGTGTCATTCTATTCTGGGGGGTGGGGTGGGGCAGGAC
AGCAAGGGGGAGGATTGGGAAGACAATAGCAGGCATGCTGGGGATGC
GGTGGGCTCTATGGAAGCTTGAATTCAGCTGACGTGCCTCGGACCGC
TAGGAACCCCTAGTGATGGAGTTGGCCACTCCCTCTCTGCGCGCTCG
CTCGCTCACTGAGGCCGGGCGACCAAAGGTCGCCCGACGCCCGGGCT
TTGCCCGGGCGGCCTCAGTGAGCGAGCGAGCGCGCAG
Exemplary pITR.CAG.miR1-26_EGFP sequence
(SEQ ID NO: 237)
CCTGCAGGCAGCTGCGCGCTCGCTCGCTCACTGAGGCCGCCCGGGCG
TCGGGCGACCTTTGGTCGCCCGGCCTCAGTGAGCGAGCGAGCGCGCA
GAGAGGGAGTGGCCAACTCCATCACTAGGGGTTCCTGCGGCCGCACG
CGTGACATTGATTATTGACTAGTTATTAATAGTAATCAATTACGGGG
TCATTAGTTCATAGCCCATATATGGAGTTCCGCGTTACATAACTTAC
GGTAAATGGCCCGCCTGGCTGACCGCCCAACGACCCCCGCCCATTGA
CGTCAATAATGACGTATGTTCCCATAGTAACGCCAATAGGGACTTTC
CATTGACGTCAATGGGTGGACTATTTACGGTAAACTGCCCACTTGGC
AGTACATCAAGTGTATCATATGCCAAGTACGCCCCCTATTGACGTCA
ATGACGGTAAATGGCCCGCCTGGCATTATGCCCAGTACATGACCTTA
TGGGACTTTCCTACTTGGCAGTACATCTACGTATTAGTCATCGCTAT
TACCATGGGTCGAGGTGAGCCCCACGTTCTGCTTCACTCTCCCCATC
TCCCCCCCCTCCCCACCCCCAATTTTGTATTTATTTATTTTTTAATT
ATTTTGTGCAGCGATGGGGGCGGGGGGGGGGGGGGCGCGCGCCAGGC
GGGGCGGGGCGGGGCGAGGGGCGGGGCGGGGCGAGGCGGAGAGGTGC
GGCGGCAGCCAATCAGAGCGGCGCGCTCCGAAAGTTTCCTTTTATGG
CGAGGCGGCGGCGGCGGCGGCCCTATAAAAAGCGAAGCGCGCGGCGG
GCGGGAGTCGCTGCGTTGCCTTCGCCCCGTGCCCCGCTCCGCGCCGC
CTCGCGCCGCCCGCCCCGGCTCTGACTGACCGCGTTACTCCCACAGG
TGAGCGGGCGGGACGGCCCTTCTCCTCCGGGCTGTAATTAGCGCTTG
GTTTAATGACGGCTCGTTTCTTTTCTGTGGCTGCGTGAAAGCCTTAA
AGGGCTCCGGGAGGGCCCTTTGTGCGGGGGGGAGCGGCTCGGGGGGT
GCGTGCGTGTGTGTGTGCGTGGGGAGCGCCGCGTGCGGCCCGCGCTG
CCCGGCGGCTGTGAGCGCTGCGGGCGCGGCGCGGGGCTTTGTGCGCT
CCGCGTGTGCGCGAGGGGAGCGCGGCCGGGGGCGGTGCCCCGCGGTG
CGGGGGGGCTGCGAGGGGAACAAAGGCTGCGTGCGGGGTGTGTGCGT
GGGGGGGTGAGCAGGGGGTGTGGGCGCGGCGGTCGGGCTGTAACCCC
CCCCTGCACCCCCCTCCCCGAGTTGCTGAGCACGGCCCGGCTTCGGG
TGCGGGGCTCCGTGCGGGGCGTGGCGCGGGGCTCGCCGTGCCGGGCG
GGGGGTGGCGGCAGGTGGGGGTGCCGGGCGGGGCGGGGCCGCCTCGG
GCCGGGGAGGGCTCGGGGGAGGGGCGCGGCGGCCCCCGGAGCGCCGG
CGGAGCAGGAGGACCAAGGCCCTGGCGAAGGCCGTGGCCTCGTTGAG
AATCCTGATGGAGCGGTGTGCAGGTCCCAATGGCCGCTCCATAAGGA
TTCTCCACGGGGACGCGGGCCTGGACGCCGGCATCCGGGCTCAGGAC
CCCCCTCTCTAGAGCCTCTGCTAACCATGTTCATGCCTTCTTCTTTT
TCCTACAGCTCCTGGGCAACGTGCTGGTTATTGTGACCGGTCGCTAG
CCACCATGGTGAGCAAGGGCGAGGAGCTGTTCACCGGGGTGGTGCCC
ATCCTGGTCGAGCTGGACGGCGACGTAAACGGCCACAAGTTCAGCGT
GTCCGGCGAGGGCGAGGGCGATGCCACCTACGGCAAGCTGACCCTGA
AGTTCATCTGCACCACCGGCAAGCTGCCCGTGCCCTGGCCCACCCTC
GTGACCACCCTGACCTACGGCGTGCAGTGCTTCAGCCGCTACCCCGA
CCACATGAAGCAGCACGACTTCTTCAAGTCCGCCATGCCCGAAGGCT
ACGTCCAGGAGCGCACCATCTTCTTCAAGGACGACGGCAACTACAAG
ACCCGCGCCGAGGTGAAGTTCGAGGGCGACACCCTGGTGAACCGCAT
CGAGCTGAAGGGCATCGACTTCAAGGAGGACGGCAACATCCTGGGGC
ACAAGCTGGAGTACAACTACAACAGCCACAACGTCTATATCATGGCC
GACAAGCAGAAGAACGGCATCAAGGTGAACTTCAAGATCCGCCACAA
CATCGAGGACGGCAGCGTGCAGCTCGCCGACCACTACCAGCAGAACA
CCCCCATCGGCGACGGCCCCGTGCTGCTGCCCGACAACCACTACCTG
AGCACCCAGTCCGCCCTGAGCAAAGACCCCAACGAGAAGCGCGATCA
CATGGTCCTGCTGGAGTTCGTGACCGCCGCCGGGATCACTCTCGGCA
TGGACGAGCTGTACAAGTAACCTGCAGGCGCCGGCGGAGCAGGAGGA
CCAAGGCCCTGGCGAAGGCCGTGGCCTCGTTGAGAATCCTGATGGAG
CGGTGTGCAGGTCCCAATGGCCGCTCCATAAGGATTCTCCACGGGGA
CGCGGGCCTGGACGCCGGCATCCGGGCTCAGGACCCCCCTCGAGCTC
GCTGATCAGCCTCGACTGTGCCTTCTAGTTGCCAGCCATCTGTTGTT
TGCCCCTCCCCCGTGCCTTCCTTGACCCTGGAAGGTGCCACTCCCAC
TGTCCTTTCCTAATAAAATGAGGAAATTGCATCGCATTGTCTGAGTA
GGTGTCATTCTATTCTGGGGGGTGGGGTGGGGCAGGACAGCAAGGGG
GAGGATTGGGAAGACAATAGCAGGCATGCTGGGGATGCGGTGGGCTC
TATGGAAGCTTGAATTCAGCTGACGTGCCTCGGACCGCTAGGAACCC
CTAGTGATGGAGTTGGCCACTCCCTCTCTGCGCGCTCGCTCGCTCAC
TGAGGCCGGGCGACCAAAGGTCGCCCGACGCCCGGGCTTTGCCCGGG
CGGCCTCAGTGAGCGAGCGAGCGCGCAG
Exemplary pITR.CAG.miR1-96_EGFP sequence
(SEQ ID NO: 238)
CCTGCAGGCAGCTGCGCGCTCGCTCGCTCACTGAGGCCGCCCGGGCG
TCGGGCGACCTTTGGTCGCCCGGCCTCAGTGAGCGAGCGAGCGCGCA
GAGAGGGAGTGGCCAACTCCATCACTAGGGGTTCCTGCGGCCGCACG
CGTGACATTGATTATTGACTAGTTATTAATAGTAATCAATTACGGGG
TCATTAGTTCATAGCCCATATATGGAGTTCCGCGTTACATAACTTAC
GGTAAATGGCCCGCCTGGCTGACCGCCCAACGACCCCCGCCCATTGA
CGTCAATAATGACGTATGTTCCCATAGTAACGCCAATAGGGACTTTC
CATTGACGTCAATGGGTGGACTATTTACGGTAAACTGCCCACTTGGC
AGTACATCAAGTGTATCATATGCCAAGTACGCCCCCTATTGACGTCA
ATGACGGTAAATGGCCCGCCTGGCATTATGCCCAGTACATGACCTTA
TGGGACTTTCCTACTTGGCAGTACATCTACGTATTAGTCATCGCTAT
TACCATGGGTCGAGGTGAGCCCCACGTTCTGCTTCACTCTCCCCATC
TCCCCCCCCTCCCCACCCCCAATTTTGTATTTATTTATTTTTTAATT
ATTTTGTGCAGCGATGGGGGCGGGGGGGGGGGGGGCGCGCGCCAGGC
GGGGCGGGGCGGGGCGAGGGGCGGGGCGGGGCGAGGCGGAGAGGTGC
GGCGGCAGCCAATCAGAGCGGCGCGCTCCGAAAGTTTCCTTTTATGG
CGAGGCGGCGGCGGCGGCGGCCCTATAAAAAGCGAAGCGCGCGGCGG
GCGGGAGTCGCTGCGTTGCCTTCGCCCCGTGCCCCGCTCCGCGCCGC
CTCGCGCCGCCCGCCCCGGCTCTGACTGACCGCGTTACTCCCACAGG
TGAGCGGGCGGGACGGCCCTTCTCCTCCGGGCTGTAATTAGCGCTTG
GTTTAATGACGGCTCGTTTCTTTTCTGTGGCTGCGTGAAAGCCTTAA
AGGGCTCCGGGAGGGCCCTTTGTGCGGGGGGGAGCGGCTCGGGGGGT
GCGTGCGTGTGTGTGTGCGTGGGGAGCGCCGCGTGCGGCCCGCGCTG
CCCGGCGGCTGTGAGCGCTGCGGGCGCGGCGCGGGGCTTTGTGCGCT
CCGCGTGTGCGCGAGGGGAGCGCGGCCGGGGGCGGTGCCCCGCGGTG
CGGGGGGGCTGCGAGGGGAACAAAGGCTGCGTGCGGGGTGTGTGCGT
GGGGGGGTGAGCAGGGGGTGTGGGCGCGGCGGTCGGGCTGTAACCCC
CCCCTGCACCCCCCTCCCCGAGTTGCTGAGCACGGCCCGGCTTCGGG
TGCGGGGCTCCGTGCGGGGCGTGGCGCGGGGCTCGCCGTGCCGGGCG
GGGGGTGGCGGCAGGTGGGGGTGCCGGGCGGGGCGGGGCCGCCTCGG
GCCGGGGAGGGCTCGGGGGAGGGGCGCGGCGGCCCCCGGAGCGCCGG
CGGTGTCCCCAGAGAGCCCGCACCAGTGCCATCTGCTTGGCCGATTT
GAGAATCCTGATGGAGCGGCTTGTGTCTCTCCGCTCTGAGCCGGGCC
ATCAGATTCTCATATGGGAAAAGCAGGACCCGCAGCTGCGTCCGCCT
CCCCTGCATTCTAGAGCCTCTGCTAACCATGTTCATGCCTTCTTCTT
TTTCCTACAGCTCCTGGGCAACGTGCTGGTTATTGTGACCGGTCGCT
AGCCACCATGGTGAGCAAGGGCGAGGAGCTGTTCACCGGGGTGGTGC
CCATCCTGGTCGAGCTGGACGGCGACGTAAACGGCCACAAGTTCAGC
GTGTCCGGCGAGGGCGAGGGCGATGCCACCTACGGCAAGCTGACCCT
GAAGTTCATCTGCACCACCGGCAAGCTGCCCGTGCCCTGGCCCACCC
TCGTGACCACCCTGACCTACGGCGTGCAGTGCTTCAGCCGCTACCCC
GACCACATGAAGCAGCACGACTTCTTCAAGTCCGCCATGCCCGAAGG
CTACGTCCAGGAGCGCACCATCTTCTTCAAGGACGACGGCAACTACA
AGACCCGCGCCGAGGTGAAGTTCGAGGGCGACACCCTGGTGAACCGC
ATCGAGCTGAAGGGCATCGACTTCAAGGAGGACGGCAACATCCTGGG
GCACAAGCTGGAGTACAACTACAACAGCCACAACGTCTATATCATGG
CCGACAAGCAGAAGAACGGCATCAAGGTGAACTTCAAGATCCGCCAC
AACATCGAGGACGGCAGCGTGCAGCTCGCCGACCACTACCAGCAGAA
CACCCCCATCGGCGACGGCCCCGTGCTGCTGCCCGACAACCACTACC
TGAGCACCCAGTCCGCCCTGAGCAAAGACCCCAACGAGAAGCGCGAT
CACATGGTCCTGCTGGAGTTCGTGACCGCCGCCGGGATCACTCTCGG
CATGGACGAGCTGTACAAGTAACCTGCAGGCGCCGGCGGTGTCCCCA
GAGAGCCCGCACCAGTGCCATCTGCTTGGCCGATTTGAGAATCCTGA
TGGAGCGGCTTGTGTCTCTCCGCTCTGAGCCGGGCCATCAGATTCTC
ATATGGGAAAAGCAGGACCCGCAGCTGCGTCCGCCTCCCCTGCATGA
GCTCGCTGATCAGCCTCGACTGTGCCTTCTAGTTGCCAGCCATCTGT
TGTTTGCCCCTCCCCCGTGCCTTCCTTGACCCTGGAAGGTGCCACTC
CCACTGTCCTTTCCTAATAAAATGAGGAAATTGCATCGCATTGTCTG
AGTAGGTGTCATTCTATTCTGGGGGGTGGGGTGGGGCAGGACAGCAA
GGGGGAGGATTGGGAAGACAATAGCAGGCATGCTGGGGATGCGGTGG
GCTCTATGGAAGCTTGAATTCAGCTGACGTGCCTCGGACCGCTAGGA
ACCCCTAGTGATGGAGTTGGCCACTCCCTCTCTGCGCGCTCGCTCGC
TCACTGAGGCCGGGCGACCAAAGGTCGCCCGACGCCCGGGCTTTGCC
CGGGCGGCCTCAGTGAGCGAGCGAGCGCGCAG
Exemplary pITR.CAG.miR1-122_EGFP sequence
(SEQ ID NO: 239)
CCTGCAGGCAGCTGCGCGCTCGCTCGCTCACTGAGGCCGCCCGGGCG
TCGGGCGACCTTTGGTCGCCCGGCCTCAGTGAGCGAGCGAGCGCGCA
GAGAGGGAGTGGCCAACTCCATCACTAGGGGTTCCTGCGGCCGCACG
CGTGACATTGATTATTGACTAGTTATTAATAGTAATCAATTACGGGG
TCATTAGTTCATAGCCCATATATGGAGTTCCGCGTTACATAACTTAC
GGTAAATGGCCCGCCTGGCTGACCGCCCAACGACCCCCGCCCATTGA
CGTCAATAATGACGTATGTTCCCATAGTAACGCCAATAGGGACTTTC
CATTGACGTCAATGGGTGGACTATTTACGGTAAACTGCCCACTTGGC
AGTACATCAAGTGTATCATATGCCAAGTACGCCCCCTATTGACGTCA
ATGACGGTAAATGGCCCGCCTGGCATTATGCCCAGTACATGACCTTA
TGGGACTTTCCTACTTGGCAGTACATCTACGTATTAGTCATCGCTAT
TACCATGGGTCGAGGTGAGCCCCACGTTCTGCTTCACTCTCCCCATC
TCCCCCCCCTCCCCACCCCCAATTTTGTATTTATTTATTTTTTAATT
ATTTTGTGCAGCGATGGGGGCGGGGGGGGGGGGGGCGCGCGCCAGGC
GGGGCGGGGCGGGGCGAGGGGCGGGGCGGGGCGAGGCGGAGAGGTGC
GGCGGCAGCCAATCAGAGCGGCGCGCTCCGAAAGTTTCCTTTTATGG
CGAGGCGGCGGCGGCGGCGGCCCTATAAAAAGCGAAGCGCGCGGCGG
GCGGGAGTCGCTGCGTTGCCTTCGCCCCGTGCCCCGCTCCGCGCCGC
CTCGCGCCGCCCGCCCCGGCTCTGACTGACCGCGTTACTCCCACAGG
TGAGCGGGCGGGACGGCCCTTCTCCTCCGGGCTGTAATTAGCGCTTG
GTTTAATGACGGCTCGTTTCTTTTCTGTGGCTGCGTGAAAGCCTTAA
AGGGCTCCGGGAGGGCCCTTTGTGCGGGGGGGAGCGGCTCGGGGGGT
GCGTGCGTGTGTGTGTGCGTGGGGAGCGCCGCGTGCGGCCCGCGCTG
CCCGGCGGCTGTGAGCGCTGCGGGCGCGGCGCGGGGCTTTGTGCGCT
CCGCGTGTGCGCGAGGGGAGCGCGGCCGGGGGCGGTGCCCCGCGGTG
CGGGGGGGCTGCGAGGGGAACAAAGGCTGCGTGCGGGGTGTGTGCGT
GGGGGGGTGAGCAGGGGGTGTGGGCGCGGCGGTCGGGCTGTAACCCC
CCCCTGCACCCCCCTCCCCGAGTTGCTGAGCACGGCCCGGCTTCGGG
TGCGGGGCTCCGTGCGGGGCGTGGCGCGGGGCTCGCCGTGCCGGGCG
GGGGGTGGCGGCAGGTGGGGGTGCCGGGCGGGGCGGGGCCGCCTCGG
GCCGGGGAGGGCTCGGGGGAGGGGCGCGGCGGCCCCCGGAGCGCCGG
CGTGAAGTTAACACCTTCGTGGCTACAGAGTTTCCTTAGCAGAGCTG
TTGAGAATCCTGATGGAGCGGGTGTCTAAACTATCCCGCTCCATCCG
GATTCTACATAGCTACTGCTAGGCAATCCTTCCCTCGATAAATGTCT
TGGCATCGTCTAGAGCCTCTGCTAACCATGTTCATGCCTTCTTCTTT
TTCCTACAGCTCCTGGGCAACGTGCTGGTTATTGTGACCGGTCGCTA
GCCACCATGGTGAGCAAGGGCGAGGAGCTGTTCACCGGGGTGGTGCC
CATCCTGGTCGAGCTGGACGGCGACGTAAACGGCCACAAGTTCAGCG
TGTCCGGCGAGGGCGAGGGCGATGCCACCTACGGCAAGCTGACCCTG
AAGTTCATCTGCACCACCGGCAAGCTGCCCGTGCCCTGGCCCACCCT
CGTGACCACCCTGACCTACGGCGTGCAGTGCTTCAGCCGCTACCCCG
ACCACATGAAGCAGCACGACTTCTTCAAGTCCGCCATGCCCGAAGGC
TACGTCCAGGAGCGCACCATCTTCTTCAAGGACGACGGCAACTACAA
GACCCGCGCCGAGGTGAAGTTCGAGGGCGACACCCTGGTGAACCGCA
TCGAGCTGAAGGGCATCGACTTCAAGGAGGACGGCAACATCCTGGGG
CACAAGCTGGAGTACAACTACAACAGCCACAACGTCTATATCATGGC
CGACAAGCAGAAGAACGGCATCAAGGTGAACTTCAAGATCCGCCACA
ACATCGAGGACGGCAGCGTGCAGCTCGCCGACCACTACCAGCAGAAC
ACCCCCATCGGCGACGGCCCCGTGCTGCTGCCCGACAACCACTACCT
GAGCACCCAGTCCGCCCTGAGCAAAGACCCCAACGAGAAGCGCGATC
ACATGGTCCTGCTGGAGTTCGTGACCGCCGCCGGGATCACTCTCGGC
ATGGACGAGCTGTACAAGTAACCTGCAGGCGCCGGCGTGAAGTTAAC
ACCTTCGTGGCTACAGAGTTTCCTTAGCAGAGCTGTTGAGAATCCTG
ATGGAGCGGGTGTCTAAACTATCCCGCTCCATCCGGATTCTACATAG
CTACTGCTAGGCAATCCTTCCCTCGATAAATGTCTTGGCATCGGAGC
TCGCTGATCAGCCTCGACTGTGCCTTCTAGTTGCCAGCCATCTGTTG
TTTGCCCCTCCCCCGTGCCTTCCTTGACCCTGGAAGGTGCCACTCCC
ACTGTCCTTTCCTAATAAAATGAGGAAATTGCATCGCATTGTCTGAG
TAGGTGTCATTCTATTCTGGGGGGTGGGGTGGGGCAGGACAGCAAGG
GGGAGGATTGGGAAGACAATAGCAGGCATGCTGGGGATGCGGTGGGC
TCTATGGAAGCTTGAATTCAGCTGACGTGCCTCGGACCGCTAGGAAC
CCCTAGTGATGGAGTTGGCCACTCCCTCTCTGCGCGCTCGCTCGCTC
ACTGAGGCCGGGCGACCAAAGGTCGCCCGACGCCCGGGCTTTGCCCG
GGCGGCCTCAGTGAGCGAGCGAGCGCGCAG
Exemplary Construct name: pITR.CAG.miR1-135_EGFP
sequence
(SEQ ID NO: 240)
CCTGCAGGCAGCTGCGCGCTCGCTCGCTCACTGAGGCCGCCCGGGCG
TCGGGCGACCTTTGGTCGCCCGGCCTCAGTGAGCGAGCGAGCGCGCA
GAGAGGGAGTGGCCAACTCCATCACTAGGGGTTCCTGCGGCCGCACG
CGTGACATTGATTATTGACTAGTTATTAATAGTAATCAATTACGGGG
TCATTAGTTCATAGCCCATATATGGAGTTCCGCGTTACATAACTTAC
GGTAAATGGCCCGCCTGGCTGACCGCCCAACGACCCCCGCCCATTGA
CGTCAATAATGACGTATGTTCCCATAGTAACGCCAATAGGGACTTTC
CATTGACGTCAATGGGTGGACTATTTACGGTAAACTGCCCACTTGGC
AGTACATCAAGTGTATCATATGCCAAGTACGCCCCCTATTGACGTCA
ATGACGGTAAATGGCCCGCCTGGCATTATGCCCAGTACATGACCTTA
TGGGACTTTCCTACTTGGCAGTACATCTACGTATTAGTCATCGCTAT
TACCATGGGTCGAGGTGAGCCCCACGTTCTGCTTCACTCTCCCCATC
TCCCCCCCCTCCCCACCCCCAATTTTGTATTTATTTATTTTTTAATT
ATTTTGTGCAGCGATGGGGGCGGGGGGGGGGGGGGCGCGCGCCAGGC
GGGGCGGGGCGGGGCGAGGGGCGGGGCGGGGCGAGGCGGAGAGGTGC
GGCGGCAGCCAATCAGAGCGGCGCGCTCCGAAAGTTTCCTTTTATGG
CGAGGCGGCGGCGGCGGCGGCCCTATAAAAAGCGAAGCGCGCGGCGG
GCGGGAGTCGCTGCGTTGCCTTCGCCCCGTGCCCCGCTCCGCGCCGC
CTCGCGCCGCCCGCCCCGGCTCTGACTGACCGCGTTACTCCCACAGG
TGAGCGGGCGGGACGGCCCTTCTCCTCCGGGCTGTAATTAGCGCTTG
GTTTAATGACGGCTCGTTTCTTTTCTGTGGCTGCGTGAAAGCCTTAA
AGGGCTCCGGGAGGGCCCTTTGTGCGGGGGGGAGCGGCTCGGGGGGT
GCGTGCGTGTGTGTGTGCGTGGGGAGCGCCGCGTGCGGCCCGCGCTG
CCCGGCGGCTGTGAGCGCTGCGGGCGCGGCGCGGGGCTTTGTGCGCT
CCGCGTGTGCGCGAGGGGAGCGCGGCCGGGGGCGGTGCCCCGCGGTG
CGGGGGGGCTGCGAGGGGAACAAAGGCTGCGTGCGGGGTGTGTGCGT
GGGGGGGTGAGCAGGGGGTGTGGGCGCGGCGGTCGGGCTGTAACCCC
CCCCTGCACCCCCCTCCCCGAGTTGCTGAGCACGGCCCGGCTTCGGG
TGCGGGGCTCCGTGCGGGGCGTGGCGCGGGGCTCGCCGTGCCGGGCG
GGGGGTGGCGGCAGGTGGGGGTGCCGGGCGGGGCGGGGCCGCCTCGG
GCCGGGGAGGGCTCGGGGGAGGGGCGCGGCGGCCCCCGGAGCGCCGG
CGGCCGCTGGACCCCTCCACTCTGCTGTGGCCTTGAGAATCCTGATG
GAGCGGGATTGCTGTCCCAAACTCCCGCTCCATCGGATTCTCAAGGC
TACAGTGAGGGGCGAGCTCCTTCTCCTGCTCTAGAGCCTCTGCTAAC
CATGTTCATGCCTTCTTCTTTTTCCTACAGCTCCTGGGCAACGTGCT
GGTTATTGTGACCGGTCGCTAGCCACCATGGTGAGCAAGGGCGAGGA
GCTGTTCACCGGGGTGGTGCCCATCCTGGTCGAGCTGGACGGCGACG
TAAACGGCCACAAGTTCAGCGTGTCCGGCGAGGGCGAGGGCGATGCC
ACCTACGGCAAGCTGACCCTGAAGTTCATCTGCACCACCGGCAAGCT
GCCCGTGCCCTGGCCCACCCTCGTGACCACCCTGACCTACGGCGTGC
AGTGCTTCAGCCGCTACCCCGACCACATGAAGCAGCACGACTTCTTC
AAGTCCGCCATGCCCGAAGGCTACGTCCAGGAGCGCACCATCTTCTT
CAAGGACGACGGCAACTACAAGACCCGCGCCGAGGTGAAGTTCGAGG
GCGACACCCTGGTGAACCGCATCGAGCTGAAGGGCATCGACTTCAAG
GAGGACGGCAACATCCTGGGGCACAAGCTGGAGTACAACTACAACAG
CCACAACGTCTATATCATGGCCGACAAGCAGAAGAACGGCATCAAGG
TGAACTTCAAGATCCGCCACAACATCGAGGACGGCAGCGTGCAGCTC
GCCGACCACTACCAGCAGAACACCCCCATCGGCGACGGCCCCGTGCT
GCTGCCCGACAACCACTACCTGAGCACCCAGTCCGCCCTGAGCAAAG
ACCCCAACGAGAAGCGCGATCACATGGTCCTGCTGGAGTTCGTGACC
GCCGCCGGGATCACTCTCGGCATGGACGAGCTGTACAAGTAACCTGC
AGGCGCCGGCGGCCGCTGGACCCCTCCACTCTGCTGTGGCCTTGAGA
ATCCTGATGGAGCGGGATTGCTGTCCCAAACTCCCGCTCCATCGGAT
TCTCAAGGCTACAGTGAGGGGCGAGCTCCTTCTCCTGCGAGCTCGCT
GATCAGCCTCGACTGTGCCTTCTAGTTGCCAGCCATCTGTTGTTTGC
CCCTCCCCCGTGCCTTCCTTGACCCTGGAAGGTGCCACTCCCACTGT
CCTTTCCTAATAAAATGAGGAAATTGCATCGCATTGTCTGAGTAGGT
GTCATTCTATTCTGGGGGGTGGGGTGGGGCAGGACAGCAAGGGGGAG
GATTGGGAAGACAATAGCAGGCATGCTGGGGATGCGGTGGGCTCTAT
GGAAGCTTGAATTCAGCTGACGTGCCTCGGACCGCTAGGAACCCCTA
GTGATGGAGTTGGCCACTCCCTCTCTGCGCGCTCGCTCGCTCACTGA
GGCCGGGCGACCAAAGGTCGCCCGACGCCCGGGCTTTGCCCGGGCGG
CCTCAGTGAGCGAGCGAGCGCGCAG
Exemplary pITR.CAG.miR1-182_EGFP sequence
(SEQ ID NO: 241)
CCTGCAGGCAGCTGCGCGCTCGCTCGCTCACTGAGGCCGCCCGGGCG
TCGGGCGACCTTTGGTCGCCCGGCCTCAGTGAGCGAGCGAGCGCGCA
GAGAGGGAGTGGCCAACTCCATCACTAGGGGTTCCTGCGGCCGCACG
CGTGACATTGATTATTGACTAGTTATTAATAGTAATCAATTACGGGG
TCATTAGTTCATAGCCCATATATGGAGTTCCGCGTTACATAACTTAC
GGTAAATGGCCCGCCTGGCTGACCGCCCAACGACCCCCGCCCATTGA
CGTCAATAATGACGTATGTTCCCATAGTAACGCCAATAGGGACTTTC
CATTGACGTCAATGGGTGGACTATTTACGGTAAACTGCCCACTTGGC
AGTACATCAAGTGTATCATATGCCAAGTACGCCCCCTATTGACGTCA
ATGACGGTAAATGGCCCGCCTGGCATTATGCCCAGTACATGACCTTA
TGGGACTTTCCTACTTGGCAGTACATCTACGTATTAGTCATCGCTAT
TACCATGGGTCGAGGTGAGCCCCACGTTCTGCTTCACTCTCCCCATC
TCCCCCCCCTCCCCACCCCCAATTTTGTATTTATTTATTTTTTAATT
ATTTTGTGCAGCGATGGGGGCGGGGGGGGGGGGGGCGCGCGCCAGGC
GGGGCGGGGCGGGGCGAGGGGCGGGGCGGGGCGAGGCGGAGAGGTGC
GGCGGCAGCCAATCAGAGCGGCGCGCTCCGAAAGTTTCCTTTTATGG
CGAGGCGGCGGCGGCGGCGGCCCTATAAAAAGCGAAGCGCGCGGCGG
GCGGGAGTCGCTGCGTTGCCTTCGCCCCGTGCCCCGCTCCGCGCCGC
CTCGCGCCGCCCGCCCCGGCTCTGACTGACCGCGTTACTCCCACAGG
TGAGCGGGCGGGACGGCCCTTCTCCTCCGGGCTGTAATTAGCGCTTG
GTTTAATGACGGCTCGTTTCTTTTCTGTGGCTGCGTGAAAGCCTTAA
AGGGCTCCGGGAGGGCCCTTTGTGCGGGGGGGAGCGGCTCGGGGGGT
GCGTGCGTGTGTGTGTGCGTGGGGAGCGCCGCGTGCGGCCCGCGCTG
CCCGGCGGCTGTGAGCGCTGCGGGCGCGGCGCGGGGCTTTGTGCGCT
CCGCGTGTGCGCGAGGGGAGCGCGGCCGGGGGCGGTGCCCCGCGGTG
CGGGGGGGCTGCGAGGGGAACAAAGGCTGCGTGCGGGGTGTGTGCGT
GGGGGGGTGAGCAGGGGGTGTGGGCGCGGCGGTCGGGCTGTAACCCC
CCCCTGCACCCCCCTCCCCGAGTTGCTGAGCACGGCCCGGCTTCGGG
TGCGGGGCTCCGTGCGGGGCGTGGCGCGGGGCTCGCCGTGCCGGGCG
GGGGGTGGCGGCAGGTGGGGGTGCCGGGCGGGGCGGGGCCGCCTCGG
GCCGGGGAGGGCTCGGGGGAGGGGCGCGGCGGCCCCCGGAGCGCCGG
CGCTCCCCAGCTCCTGGGGGGAGCTGCTTGCCTCCCCCCGTTTTGAG
AATCCTGATGGAGCGGACTGGTGAGGTAACAGGATCCGCCGTCCATC
TTTATTCTCACTATGGGGCGAGGACTCAGCCGGCACCCTGTGCACAG
CCAGCGAGGGTCTAGAGCCTCTGCTAACCATGTTCATGCCTTCTTCT
TTTTCCTACAGCTCCTGGGCAACGTGCTGGTTATTGTGACCGGTCGC
TAGCCACCATGGTGAGCAAGGGCGAGGAGCTGTTCACCGGGGTGGTG
CCCATCCTGGTCGAGCTGGACGGCGACGTAAACGGCCACAAGTTCAG
CGTGTCCGGCGAGGGCGAGGGCGATGCCACCTACGGCAAGCTGACCC
TGAAGTTCATCTGCACCACCGGCAAGCTGCCCGTGCCCTGGCCCACC
CTCGTGACCACCCTGACCTACGGCGTGCAGTGCTTCAGCCGCTACCC
CGACCACATGAAGCAGCACGACTTCTTCAAGTCCGCCATGCCCGAAG
GCTACGTCCAGGAGCGCACCATCTTCTTCAAGGACGACGGCAACTAC
AAGACCCGCGCCGAGGTGAAGTTCGAGGGCGACACCCTGGTGAACCG
CATCGAGCTGAAGGGCATCGACTTCAAGGAGGACGGCAACATCCTGG
GGCACAAGCTGGAGTACAACTACAACAGCCACAACGTCTATATCATG
GCCGACAAGCAGAAGAACGGCATCAAGGTGAACTTCAAGATCCGCCA
CAACATCGAGGACGGCAGCGTGCAGCTCGCCGACCACTACCAGCAGA
ACACCCCCATCGGCGACGGCCCCGTGCTGCTGCCCGACAACCACTAC
CTGAGCACCCAGTCCGCCCTGAGCAAAGACCCCAACGAGAAGCGCGA
TCACATGGTCCTGCTGGAGTTCGTGACCGCCGCCGGGATCACTCTCG
GCATGGACGAGCTGTACAAGTAACCTGCAGGCGCCGGCGCTCCCCAG
CTCCTGGGGGGAGCTGCTTGCCTCCCCCCGTTTTGAGAATCCTGATG
GAGCGGACTGGTGAGGTAACAGGATCCGCCGTCCATCTTTATTCTCA
CTATGGGGCGAGGACTCAGCCGGCACCCTGTGCACAGCCAGCGAGGG
GAGCTCGCTGATCAGCCTCGACTGTGCCTTCTAGTTGCCAGCCATCT
GTTGTTTGCCCCTCCCCCGTGCCTTCCTTGACCCTGGAAGGTGCCAC
TCCCACTGTCCTTTCCTAATAAAATGAGGAAATTGCATCGCATTGTC
TGAGTAGGTGTCATTCTATTCTGGGGGGTGGGGTGGGGCAGGACAGC
AAGGGGGAGGATTGGGAAGACAATAGCAGGCATGCTGGGGATGCGGT
GGGCTCTATGGAAGCTTGAATTCAGCTGACGTGCCTCGGACCGCTAG
GAACCCCTAGTGATGGAGTTGGCCACTCCCTCTCTGCGCGCTCGCTC
GCTCACTGAGGCCGGGCGACCAAAGGTCGCCCGACGCCCGGGCTTTG
CCCGGGCGGCCTCAGTGAGCGAGCGAGCGCGCAG
Exemplary pITR.CAG.miR1-183_EGFP sequence
(SEQ ID NO: 242)
CCTGCAGGCAGCTGCGCGCTCGCTCGCTCACTGAGGCCGCCCGGGCG
TCGGGCGACCTTTGGTCGCCCGGCCTCAGTGAGCGAGCGAGCGCGCA
GAGAGGGAGTGGCCAACTCCATCACTAGGGGTTCCTGCGGCCGCACG
CGTGACATTGATTATTGACTAGTTATTAATAGTAATCAATTACGGGG
TCATTAGTTCATAGCCCATATATGGAGTTCCGCGTTACATAACTTAC
GGTAAATGGCCCGCCTGGCTGACCGCCCAACGACCCCCGCCCATTGA
CGTCAATAATGACGTATGTTCCCATAGTAACGCCAATAGGGACTTTC
CATTGACGTCAATGGGTGGACTATTTACGGTAAACTGCCCACTTGGC
AGTACATCAAGTGTATCATATGCCAAGTACGCCCCCTATTGACGTCA
ATGACGGTAAATGGCCCGCCTGGCATTATGCCCAGTACATGACCTTA
TGGGACTTTCCTACTTGGCAGTACATCTACGTATTAGTCATCGCTAT
TACCATGGGTCGAGGTGAGCCCCACGTTCTGCTTCACTCTCCCCATC
TCCCCCCCCTCCCCACCCCCAATTTTGTATTTATTTATTTTTTAATT
ATTTTGTGCAGCGATGGGGGCGGGGGGGGGGGGGGCGCGCGCCAGGC
GGGGCGGGGCGGGGCGAGGGGCGGGGCGGGGCGAGGCGGAGAGGTGC
GGCGGCAGCCAATCAGAGCGGCGCGCTCCGAAAGTTTCCTTTTATGG
CGAGGCGGCGGCGGCGGCGGCCCTATAAAAAGCGAAGCGCGCGGCGG
GCGGGAGTCGCTGCGTTGCCTTCGCCCCGTGCCCCGCTCCGCGCCGC
CTCGCGCCGCCCGCCCCGGCTCTGACTGACCGCGTTACTCCCACAGG
TGAGCGGGCGGGACGGCCCTTCTCCTCCGGGCTGTAATTAGCGCTTG
GTTTAATGACGGCTCGTTTCTTTTCTGTGGCTGCGTGAAAGCCTTAA
AGGGCTCCGGGAGGGCCCTTTGTGCGGGGGGGAGCGGCTCGGGGGGT
GCGTGCGTGTGTGTGTGCGTGGGGAGCGCCGCGTGCGGCCCGCGCTG
CCCGGCGGCTGTGAGCGCTGCGGGCGCGGCGCGGGGCTTTGTGCGCT
CCGCGTGTGCGCGAGGGGAGCGCGGCCGGGGGCGGTGCCCCGCGGTG
CGGGGGGGCTGCGAGGGGAACAAAGGCTGCGTGCGGGGTGTGTGCGT
GGGGGGGTGAGCAGGGGGTGTGGGCGCGGCGGTCGGGCTGTAACCCC
CCCCTGCACCCCCCTCCCCGAGTTGCTGAGCACGGCCCGGCTTCGGG
TGCGGGGCTCCGTGCGGGGCGTGGCGCGGGGCTCGCCGTGCCGGGCG
GGGGGTGGCGGCAGGTGGGGGTGCCGGGCGGGGCGGGGCCGCCTCGG
GCCGGGGAGGGCTCGGGGGAGGGGCGCGGCGGCCCCCGGAGCGCCGG
CGAGGGTCGGCAGGCCGCAGAGTGTGACTCCTGTTCTGTGTTTGAGA
ATCCTGATGGAGCGGGTGAACAGTCTCAGTCCCGCTCCCAGGAAATG
GTCAAAAACAGAGCAGAGACAGATCCACGAGGGCCTCCGGAGCACCT
TTCTAGAGCCTCTGCTAACCATGTTCATGCCTTCTTCTTTTTCCTAC
AGCTCCTGGGCAACGTGCTGGTTATTGTGACCGGTCGCTAGCCACCA
TGGTGAGCAAGGGCGAGGAGCTGTTCACCGGGGTGGTGCCCATCCTG
GTCGAGCTGGACGGCGACGTAAACGGCCACAAGTTCAGCGTGTCCGG
CGAGGGCGAGGGCGATGCCACCTACGGCAAGCTGACCCTGAAGTTCA
TCTGCACCACCGGCAAGCTGCCCGTGCCCTGGCCCACCCTCGTGACC
ACCCTGACCTACGGCGTGCAGTGCTTCAGCCGCTACCCCGACCACAT
GAAGCAGCACGACTTCTTCAAGTCCGCCATGCCCGAAGGCTACGTCC
AGGAGCGCACCATCTTCTTCAAGGACGACGGCAACTACAAGACCCGC
GCCGAGGTGAAGTTCGAGGGCGACACCCTGGTGAACCGCATCGAGCT
GAAGGGCATCGACTTCAAGGAGGACGGCAACATCCTGGGGCACAAGC
TGGAGTACAACTACAACAGCCACAACGTCTATATCATGGCCGACAAG
CAGAAGAACGGCATCAAGGTGAACTTCAAGATCCGCCACAACATCGA
GGACGGCAGCGTGCAGCTCGCCGACCACTACCAGCAGAACACCCCCA
TCGGCGACGGCCCCGTGCTGCTGCCCGACAACCACTACCTGAGCACC
CAGTCCGCCCTGAGCAAAGACCCCAACGAGAAGCGCGATCACATGGT
CCTGCTGGAGTTCGTGACCGCCGCCGGGATCACTCTCGGCATGGACG
AGCTGTACAAGTAACCTGCAGGCGCCGGCGAGGGTCGGCAGGCCGCA
GAGTGTGACTCCTGTTCTGTGTTTGAGAATCCTGATGGAGCGGGTGA
ACAGTCTCAGTCCCGCTCCCAGGAAATGGTCAAAAACAGAGCAGAGA
CAGATCCACGAGGGCCTCCGGAGCACCTTGAGCTCGCTGATCAGCCT
CGACTGTGCCTTCTAGTTGCCAGCCATCTGTTGTTTGCCCCTCCCCC
GTGCCTTCCTTGACCCTGGAAGGTGCCACTCCCACTGTCCTTTCCTA
ATAAAATGAGGAAATTGCATCGCATTGTCTGAGTAGGTGTCATTCTA
TTCTGGGGGGTGGGGTGGGGCAGGACAGCAAGGGGGAGGATTGGGAA
GACAATAGCAGGCATGCTGGGGATGCGGTGGGCTCTATGGAAGCTTG
AATTCAGCTGACGTGCCTCGGACCGCTAGGAACCCCTAGTGATGGAG
TTGGCCACTCCCTCTCTGCGCGCTCGCTCGCTCACTGAGGCCGGGCG
ACCAAAGGTCGCCCGACGCCCGGGCTTTGCCCGGGCGGCCTCAGTGA
GCGAGCGAGCGCGCAG
Exemplary pITR.CAG.miR1-335_EGFP sequence
(SEQ ID NO: 243)
CCTGCAGGCAGCTGCGCGCTCGCTCGCTCACTGAGGCCGCCCGGGCG
TCGGGCGACCTTTGGTCGCCCGGCCTCAGTGAGCGAGCGAGCGCGCA
GAGAGGGAGTGGCCAACTCCATCACTAGGGGTTCCTGCGGCCGCACG
CGTGACATTGATTATTGACTAGTTATTAATAGTAATCAATTACGGGG
TCATTAGTTCATAGCCCATATATGGAGTTCCGCGTTACATAACTTAC
GGTAAATGGCCCGCCTGGCTGACCGCCCAACGACCCCCGCCCATTGA
CGTCAATAATGACGTATGTTCCCATAGTAACGCCAATAGGGACTTTC
CATTGACGTCAATGGGTGGACTATTTACGGTAAACTGCCCACTTGGC
AGTACATCAAGTGTATCATATGCCAAGTACGCCCCCTATTGACGTCA
ATGACGGTAAATGGCCCGCCTGGCATTATGCCCAGTACATGACCTTA
TGGGACTTTCCTACTTGGCAGTACATCTACGTATTAGTCATCGCTAT
TACCATGGGTCGAGGTGAGCCCCACGTTCTGCTTCACTCTCCCCATC
TCCCCCCCCTCCCCACCCCCAATTTTGTATTTATTTATTTTTTAATT
ATTTTGTGCAGCGATGGGGGCGGGGGGGGGGGGGGCGCGCGCCAGGC
GGGGCGGGGCGGGGCGAGGGGCGGGGCGGGGCGAGGCGGAGAGGTGC
GGCGGCAGCCAATCAGAGCGGCGCGCTCCGAAAGTTTCCTTTTATGG
CGAGGCGGCGGCGGCGGCGGCCCTATAAAAAGCGAAGCGCGCGGCGG
GCGGGAGTCGCTGCGTTGCCTTCGCCCCGTGCCCCGCTCCGCGCCGC
CTCGCGCCGCCCGCCCCGGCTCTGACTGACCGCGTTACTCCCACAGG
TGAGCGGGCGGGACGGCCCTTCTCCTCCGGGCTGTAATTAGCGCTTG
GTTTAATGACGGCTCGTTTCTTTTCTGTGGCTGCGTGAAAGCCTTAA
AGGGCTCCGGGAGGGCCCTTTGTGCGGGGGGGAGCGGCTCGGGGGGT
GCGTGCGTGTGTGTGTGCGTGGGGAGCGCCGCGTGCGGCCCGCGCTG
CCCGGCGGCTGTGAGCGCTGCGGGCGCGGCGCGGGGCTTTGTGCGCT
CCGCGTGTGCGCGAGGGGAGCGCGGCCGGGGGCGGTGCCCCGCGGTG
CGGGGGGGCTGCGAGGGGAACAAAGGCTGCGTGCGGGGTGTGTGCGT
GGGGGGGTGAGCAGGGGGTGTGGGCGCGGCGGTCGGGCTGTAACCCC
CCCCTGCACCCCCCTCCCCGAGTTGCTGAGCACGGCCCGGCTTCGGG
TGCGGGGCTCCGTGCGGGGCGTGGCGCGGGGCTCGCCGTGCCGGGCG
GGGGGTGGCGGCAGGTGGGGGTGCCGGGCGGGGCGGGGCCGCCTCGG
GCCGGGGAGGGCTCGGGGGAGGGGCGCGGCGGCCCCCGGAGCGCCGG
CGGAAACAGATTGGAAATGATTTGTTTTGAGCGGGGGTTGAGAATCC
TGATGGAGCGGGTTTGTCATAAACCCCGCTCCCTCAGGATTCCCAAC
CTCCTCTCATTTGCTATATTCAATTAAGTAATCTAGAGCCTCTGCTA
ACCATGTTCATGCCTTCTTCTTTTTCCTACAGCTCCTGGGCAACGTG
CTGGTTATTGTGACCGGTCGCTAGCCACCATGGTGAGCAAGGGCGAG
GAGCTGTTCACCGGGGTGGTGCCCATCCTGGTCGAGCTGGACGGCGA
CGTAAACGGCCACAAGTTCAGCGTGTCCGGCGAGGGCGAGGGCGATG
CCACCTACGGCAAGCTGACCCTGAAGTTCATCTGCACCACCGGCAAG
CTGCCCGTGCCCTGGCCCACCCTCGTGACCACCCTGACCTACGGCGT
GCAGTGCTTCAGCCGCTACCCCGACCACATGAAGCAGCACGACTTCT
TCAAGTCCGCCATGCCCGAAGGCTACGTCCAGGAGCGCACCATCTTC
TTCAAGGACGACGGCAACTACAAGACCCGCGCCGAGGTGAAGTTCGA
GGGCGACACCCTGGTGAACCGCATCGAGCTGAAGGGCATCGACTTCA
AGGAGGACGGCAACATCCTGGGGCACAAGCTGGAGTACAACTACAAC
AGCCACAACGTCTATATCATGGCCGACAAGCAGAAGAACGGCATCAA
GGTGAACTTCAAGATCCGCCACAACATCGAGGACGGCAGCGTGCAGC
TCGCCGACCACTACCAGCAGAACACCCCCATCGGCGACGGCCCCGTG
CTGCTGCCCGACAACCACTACCTGAGCACCCAGTCCGCCCTGAGCAA
AGACCCCAACGAGAAGCGCGATCACATGGTCCTGCTGGAGTTCGTGA
CCGCCGCCGGGATCACTCTCGGCATGGACGAGCTGTACAAGTAACCT
GCAGGCGCCGGCGGAAACAGATTGGAAATGATTTGTTTTGAGCGGGG
GTTGAGAATCCTGATGGAGCGGGTTTGTCATAAACCCCGCTCCCTCA
GGATTCCCAACCTCCTCTCATTTGCTATATTCAATTAAGTAAGAGCT
CGCTGATCAGCCTCGACTGTGCCTTCTAGTTGCCAGCCATCTGTTGT
TTGCCCCTCCCCCGTGCCTTCCTTGACCCTGGAAGGTGCCACTCCCA
CTGTCCTTTCCTAATAAAATGAGGAAATTGCATCGCATTGTCTGAGT
AGGTGTCATTCTATTCTGGGGGGTGGGGTGGGGCAGGACAGCAAGGG
GGAGGATTGGGAAGACAATAGCAGGCATGCTGGGGATGCGGTGGGCT
CTATGGAAGCTTGAATTCAGCTGACGTGCCTCGGACCGCTAGGAACC
CCTAGTGATGGAGTTGGCCACTCCCTCTCTGCGCGCTCGCTCGCTCA
CTGAGGCCGGGCGACCAAAGGTCGCCCGACGCCCGGGCTTTGCCCGG
GCGGCCTCAGTGAGCGAGCGAGCGCGCAG
Exemplary pITR.CAG.miR1-451_EGFP sequence
(SEQ ID NO: 244)
CCTGCAGGCAGCTGCGCGCTCGCTCGCTCACTGAGGCCGCCCGGGCG
TCGGGCGACCTTTGGTCGCCCGGCCTCAGTGAGCGAGCGAGCGCGCA
GAGAGGGAGTGGCCAACTCCATCACTAGGGGTTCCTGCGGCCGCACG
CGTGACATTGATTATTGACTAGTTATTAATAGTAATCAATTACGGGG
TCATTAGTTCATAGCCCATATATGGAGTTCCGCGTTACATAACTTAC
GGTAAATGGCCCGCCTGGCTGACCGCCCAACGACCCCCGCCCATTGA
CGTCAATAATGACGTATGTTCCCATAGTAACGCCAATAGGGACTTTC
CATTGACGTCAATGGGTGGACTATTTACGGTAAACTGCCCACTTGGC
AGTACATCAAGTGTATCATATGCCAAGTACGCCCCCTATTGACGTCA
ATGACGGTAAATGGCCCGCCTGGCATTATGCCCAGTACATGACCTTA
TGGGACTTTCCTACTTGGCAGTACATCTACGTATTAGTCATCGCTAT
TACCATGGGTCGAGGTGAGCCCCACGTTCTGCTTCACTCTCCCCATC
TCCCCCCCCTCCCCACCCCCAATTTTGTATTTATTTATTTTTTAATT
ATTTTGTGCAGCGATGGGGGCGGGGGGGGGGGGGGCGCGCGCCAGGC
GGGGCGGGGCGGGGCGAGGGGCGGGGCGGGGCGAGGCGGAGAGGTGC
GGCGGCAGCCAATCAGAGCGGCGCGCTCCGAAAGTTTCCTTTTATGG
CGAGGCGGCGGCGGCGGCGGCCCTATAAAAAGCGAAGCGCGCGGCGG
GCGGGAGTCGCTGCGTTGCCTTCGCCCCGTGCCCCGCTCCGCGCCGC
CTCGCGCCGCCCGCCCCGGCTCTGACTGACCGCGTTACTCCCACAGG
TGAGCGGGCGGGACGGCCCTTCTCCTCCGGGCTGTAATTAGCGCTTG
GTTTAATGACGGCTCGTTTCTTTTCTGTGGCTGCGTGAAAGCCTTAA
AGGGCTCCGGGAGGGCCCTTTGTGCGGGGGGGAGCGGCTCGGGGGGT
GCGTGCGTGTGTGTGTGCGTGGGGAGCGCCGCGTGCGGCCCGCGCTG
CCCGGCGGCTGTGAGCGCTGCGGGCGCGGCGCGGGGCTTTGTGCGCT
CCGCGTGTGCGCGAGGGGAGCGCGGCCGGGGGCGGTGCCCCGCGGTG
CGGGGGGGCTGCGAGGGGAACAAAGGCTGCGTGCGGGGTGTGTGCGT
GGGGGGGTGAGCAGGGGGTGTGGGCGCGGCGGTCGGGCTGTAACCCC
CCCCTGCACCCCCCTCCCCGAGTTGCTGAGCACGGCCCGGCTTCGGG
TGCGGGGCTCCGTGCGGGGCGTGGCGCGGGGCTCGCCGTGCCGGGCG
GGGGGTGGCGGCAGGTGGGGGTGCCGGGCGGGGCGGGGCCGCCTCGG
GCCGGGGAGGGCTCGGGGGAGGGGCGCGGCGGCCCCCGGAGCGCCGG
CGTGACTGCCAGGGCACTTGGGAATGGCAAGGTTGAGAATCCTGATG
GAGCGGATTCCATCAGGATTCTCAGTCTTGCTATACCCAGAAAACGT
GCCAGGAAGATCTAGAGCCTCTGCTAACCATGTTCATGCCTTCTTCT
TTTTCCTACAGCTCCTGGGCAACGTGCTGGTTATTGTGACCGGTCGC
TAGCCACCATGGTGAGCAAGGGCGAGGAGCTGTTCACCGGGGTGGTG
CCCATCCTGGTCGAGCTGGACGGCGACGTAAACGGCCACAAGTTCAG
CGTGTCCGGCGAGGGCGAGGGCGATGCCACCTACGGCAAGCTGACCC
TGAAGTTCATCTGCACCACCGGCAAGCTGCCCGTGCCCTGGCCCACC
CTCGTGACCACCCTGACCTACGGCGTGCAGTGCTTCAGCCGCTACCC
CGACCACATGAAGCAGCACGACTTCTTCAAGTCCGCCATGCCCGAAG
GCTACGTCCAGGAGCGCACCATCTTCTTCAAGGACGACGGCAACTAC
AAGACCCGCGCCGAGGTGAAGTTCGAGGGCGACACCCTGGTGAACCG
CATCGAGCTGAAGGGCATCGACTTCAAGGAGGACGGCAACATCCTGG
GGCACAAGCTGGAGTACAACTACAACAGCCACAACGTCTATATCATG
GCCGACAAGCAGAAGAACGGCATCAAGGTGAACTTCAAGATCCGCCA
CAACATCGAGGACGGCAGCGTGCAGCTCGCCGACCACTACCAGCAGA
ACACCCCCATCGGCGACGGCCCCGTGCTGCTGCCCGACAACCACTAC
CTGAGCACCCAGTCCGCCCTGAGCAAAGACCCCAACGAGAAGCGCGA
TCACATGGTCCTGCTGGAGTTCGTGACCGCCGCCGGGATCACTCTCG
GCATGGACGAGCTGTACAAGTAACCTGCAGGCGCCGGCGTGACTGCC
AGGGCACTTGGGAATGGCAAGGTTGAGAATCCTGATGGAGCGGATTC
CATCAGGATTCTCAGTCTTGCTATACCCAGAAAACGTGCCAGGAAGA
GAGCTCGCTGATCAGCCTCGACTGTGCCTTCTAGTTGCCAGCCATCT
GTTGTTTGCCCCTCCCCCGTGCCTTCCTTGACCCTGGAAGGTGCCAC
TCCCACTGTCCTTTCCTAATAAAATGAGGAAATTGCATCGCATTGTC
TGAGTAGGTGTCATTCTATTCTGGGGGGTGGGGTGGGGCAGGACAGC
AAGGGGGAGGATTGGGAAGACAATAGCAGGCATGCTGGGGATGCGGT
GGGCTCTATGGAAGCTTGAATTCAGCTGACGTGCCTCGGACCGCTAG
GAACCCCTAGTGATGGAGTTGGCCACTCCCTCTCTGCGCGCTCGCTC
GCTCACTGAGGCCGGGCGACCAAAGGTCGCCCGACGCCCGGGCTTTG
CCCGGGCGGCCTCAGTGAGCGAGCGAGCGCGCAG

Exemplary miRNA Construct Sequences (Part 4)

Exemplary pITR.CAG.mmu.miR6-26 EGFP
(SEQ ID NO: 245)
CTGCGCGCTCGCTCGCTCACTGAGGCCGCCCGGGCGTCGGGCGACCTTTGGTCGCCCGGCCTCA
GTGAGCGAGCGAGCGCGCAGAGAGGGAGTGGCCAACTCCATCACTAGGGGTTCCTGCGGCCGCA
CGCGTGACATTGATTATTGACTAGTTATTAATAGTAATCAATTACGGGGTCATTAGTTCATAGC
CCATATATGGAGTTCCGCGTTACATAACTTACGGTAAATGGCCCGCCTGGCTGACCGCCCAACG
ACCCCCGCCCATTGACGTCAATAATGACGTATGTTCCCATAGTAACGCCAATAGGGACTTTCCA
TTGACGTCAATGGGTGGACTATTTACGGTAAACTGCCCACTTGGCAGTACATCAAGTGTATCAT
ATGCCAAGTACGCCCCCTATTGACGTCAATGACGGTAAATGGCCCGCCTGGCATTATGCCCAGT
ACATGACCTTATGGGACTTTCCTACTTGGCAGTACATCTACGTATTAGTCATCGCTATTACCAT
GGGTCGAGGTGAGCCCCACGTTCTGCTTCACTCTCCCCATCTCCCCCCCCTCCCCACCCCCAAT
TTTGTATTTATTTATTTTTTAATTATTTTGTGCAGCGATGGGGGCGGGGGGGGGGGGGGCGCGC
GCCAGGCGGGGCGGGGCGGGGCGAGGGGCGGGGCGGGGCGAGGCGGAGAGGTGCGGCGGCAGCC
AATCAGAGCGGCGCGCTCCGAAAGTTTCCTTTTATGGCGAGGCGGCGGCGGCGGCGGCCCTATA
AAAAGCGAAGCGCGCGGCGGGCGGGAGTCGCTGCGTTGCCTTCGCCCCGTGCCCCGCTCCGCGC
CGCCTCGCGCCGCCCGCCCCGGCTCTGACTGACCGCGTTACTCCCACAGGTGAGCGGGCGGGAC
GGCCCTTCTCCTCCGGGCTGTAATTAGCGCTTGGTTTAATGACGGCTCGTTTCTTTTCTGTGGC
TGCGTGAAAGCCTTAAAGGGCTCCGGGAGGGCCCTTTGTGCGGGGGGGAGCGGCTCGGGGGGTG
CGTGCGTGTGTGTGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGCGGCCGGGGGCGG
TGCCCCGCGGTGCGGGGGGGCTGCGAGGGGAACAAAGGCTGCGTGCGGGGTGTGTGCGTGGGGG
GGTGAGCAGGGGGTGTGGGCGCGGCGGTCGGGCTGTAACCCCCCCCTGCACCCCCCTCCCCGAG
TTGCTGAGCACGGCCCGGCTTCGGGTGCGGGGCTCCGTGCGGGGCGTGGCGCGGGGCTCGCCGT
GCCGGGCGGGGGGTGGCGGCAGGTGGGGGTGCCGGGCGGGGCGGGGCCGCCTCGGGCCGGGGAG
GGCTCGGGGGAGGGGCGCGGCGGCCCCCGGAGCGCCGGCGGAGCAGGAGGACCAAGGCCCTGGC
GAAGGCCGTGGCCTCGATGCTGTCATAGTAATACCAGTGTGCAGGTCCCAATGGCTGGTATTAA
TGTGACAGTCTCGGGGACGCGGGCCTGGACGCCGGCATCCGGGCTCAGGACCCCCCTCTCTAGA
GCCTCTGCTAACCATGTTCATGCCTTCTTCTTTTTCCTACAGCTCCTGGGCAACGTGCTGGTTA
TTGTGACCGGTCGCTAGCCACCATGGTGAGCAAGGGCGAGGAGCTGTTCACCGGGGTGGTGCCC
ATCCTGGTCGAGCTGGACGGCGACGTAAACGGCCACAAGTTCAGCGTGTCCGGCGAGGGCGAGG
GCGATGCCACCTACGGCAAGCTGACCCTGAAGTTCATCTGCACCACCGGCAAGCTGCCCGTGCC
CTGGCCCACCCTCGTGACCACCCTGACCTACGGCGTGCAGTGCTTCAGCCGCTACCCCGACCAC
ATGAAGCAGCACGACTTCTTCAAGTCCGCCATGCCCGAAGGCTACGTCCAGGAGCGCACCATCT
TCTTCAAGGACGACGGCAACTACAAGACCCGCGCCGAGGTGAAGTTCGAGGGCGACACCCTGGT
GAACCGCATCGAGCTGAAGGGCATCGACTTCAAGGAGGACGGCAACATCCTGGGGCACAAGCTG
GAGTACAACTACAACAGCCACAACGTCTATATCATGGCCGACAAGCAGAAGAACGGCATCAAGG
TGAACTTCAAGATCCGCCACAACATCGAGGACGGCAGCGTGCAGCTCGCCGACCACTACCAGCA
GAACACCCCCATCGGCGACGGCCCCGTGCTGCTGCCCGACAACCACTACCTGAGCACCCAGTCC
GCCCTGAGCAAAGACCCCAACGAGAAGCGCGATCACATGGTCCTGCTGGAGTTCGTGACCGCCG
CCGGGATCACTCTCGGCATGGACGAGCTGTACAAGTAAcctgcaggcgccggcgGAGCAGGAGG
ACCAAGGCCCTGGCGAAGGCCGTGGCCTCGATGCTGTCATAGTAATACCAGTGTGCAGGTCCCA
ATGGCTGGTATTAATGTGACAGTCTCGGGGACGCGGGCCTGGACGCCGGCATCCGGGCTCAGGA
CCCCCCTCGAGCTCGCTGATCAGCCTCGACTGTGCCTTCTAGTTGCCAGCCATCTGTTGTTTGC
CCCTCCCCCGTGCCTTCCTTGACCCTGGAAGGTGCCACTCCCACTGTCCTTTCCTAATAAAATG
AGGAAATTGCATCGCATTGTCTGAGTAGGTGTCATTCTATTCTGGGGGGTGGGGTGGGGCAGGA
CAGCAAGGGGGAGGATTGGGAAGACAATAGCAGGCATGCTGGGGATGCGGTGGGCTCTATGGAA
GCTTGAATTCAGCTGACGTGCCTCGGACCGCTAGGAACCCCTAGTGATGGAGTTGGCCACTCCC
TCTCTGCGCGCTCGCTCGCTCACTGAGGCCGGGCGACCAAAGGTCGCCCGACGCCCGGGCTTTG
CCCGGGCGGCCTCAGTGAGCGAGCGAGCGCGCAG
Exemplary pITR.CAG.mmu.miR6-26_KCNQ4codop
(SEQ ID NO: 246)
CTGCGCGCTCGCTCGCTCACTGAGGCCGCCCGGGCGTCGGGCGACCTTTGGTCGCCCGGCCTCA
GTGAGCGAGCGAGCGCGCAGAGAGGGAGTGGCCAACTCCATCACTAGGGGTTCCTGCGGCCGCA
CGCGTGACATTGATTATTGACTAGTTATTAATAGTAATCAATTACGGGGTCATTAGTTCATAGC
CCATATATGGAGTTCCGCGTTACATAACTTACGGTAAATGGCCCGCCTGGCTGACCGCCCAACG
ACCCCCGCCCATTGACGTCAATAATGACGTATGTTCCCATAGTAACGCCAATAGGGACTTTCCA
TTGACGTCAATGGGTGGACTATTTACGGTAAACTGCCCACTTGGCAGTACATCAAGTGTATCAT
ATGCCAAGTACGCCCCCTATTGACGTCAATGACGGTAAATGGCCCGCCTGGCATTATGCCCAGT
ACATGACCTTATGGGACTTTCCTACTTGGCAGTACATCTACGTATTAGTCATCGCTATTACCAT
GGGTCGAGGTGAGCCCCACGTTCTGCTTCACTCTCCCCATCTCCCCCCCCTCCCCACCCCCAAT
TTTGTATTTATTTATTTTTTAATTATTTTGTGCAGCGATGGGGGCGGGGGGGGGGGGGGCGCGC
GCCAGGCGGGGCGGGGCGGGGCGAGGGGCGGGGCGGGGCGAGGCGGAGAGGTGCGGCGGCAGCC
AATCAGAGCGGCGCGCTCCGAAAGTTTCCTTTTATGGCGAGGCGGCGGCGGCGGCGGCCCTATA
AAAAGCGAAGCGCGCGGCGGGCGGGAGTCGCTGCGTTGCCTTCGCCCCGTGCCCCGCTCCGCGC
CGCCTCGCGCCGCCCGCCCCGGCTCTGACTGACCGCGTTACTCCCACAGGTGAGCGGGCGGGAC
GGCCCTTCTCCTCCGGGCTGTAATTAGCGCTTGGTTTAATGACGGCTCGTTTCTTTTCTGTGGC
TGCGTGAAAGCCTTAAAGGGCTCCGGGAGGGCCCTTTGTGCGGGGGGGAGCGGCTCGGGGGGTG
CGTGCGTGTGTGTGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGCGGCCGGGGGCGG
TGCCCCGCGGTGCGGGGGGGCTGCGAGGGGAACAAAGGCTGCGTGCGGGGTGTGTGCGTGGGGG
GGTGAGCAGGGGGTGTGGGCGCGGCGGTCGGGCTGTAACCCCCCCCTGCACCCCCCTCCCCGAG
TTGCTGAGCACGGCCCGGCTTCGGGTGCGGGGCTCCGTGCGGGGCGTGGCGCGGGGCTCGCCGT
GCCGGGCGGGGGGTGGCGGCAGGTGGGGGTGCCGGGCGGGGCGGGGCCGCCTCGGGCCGGGGAG
GGCTCGGGGGAGGGGCGCGGCGGCCCCCGGAGCGCCGGCGGAGCAGGAGGACCAAGGCCCTGGC
GAAGGCCGTGGCCTCGATGCTGTCATAGTAATACCAGTGTGCAGGTCCCAATGGCTGGTATTAA
TGTGACAGTCTCGGGGACGCGGGCCTGGACGCCGGCATCCGGGCTCAGGACCCCCCTCTCTAGA
GCCTCTGCTAACCATGTTCATGCCTTCTTCTTTTTCCTACAGCTCCTGGGCAACGTGCTGGTTA
TTGTGACCGGTCGCTAGCCACCATGGCTGAAGCCCCTCCTAGAAGGCTTGGACTGGGACCTCCT
CCTGGGGATGCTCCTAGAGCTGAACTGGTGGCTCTGACAGCCGTGCAGTCTGAACAAGGCGAAG
CTGGTGGCGGCGGATCTCCACGTAGACTTGGACTGCTGGGAAGCCCTCTTCCTCCTGGTGCTCC
ACTTCCTGGACCTGGCAGTGGATCTGGATCTGCCTGTGGCCAGAGAAGCTCTGCCGCTCACAAG
AGATACCGGCGGCTGCAGAACTGGGTGTACAACGTGCTGGAAAGACCCAGAGGCTGGGCCTTCG
TGTACCACGTGTTCATCTTTCTGCTGGTGTTCAGCTGCCTGGTGCTGTCCGTGCTGAGCACCAT
CCAAGAACATCAAGAGCTGGCTAACGAGTGCCTGTTAATACTGGAGTTTGTGATGATTGTGGTG
TTCGGCCTCGAGTACATCGTCCGCGTTTGGAGCGCCGGCTGCTGCTGCAGATATAGAGGTTGGC
AAGGCAGATTCCGCTTCGCCAGAAAGCCCTTCTGCGTGATCGACTTCATCGTGTTCGTGGCCAG
CGTGGCCGTGATTGCTGCTGGCACACAGGGCAACATCTTCGCCACAAGCGCCCTGCGGAGCATG
CGGTTTCTGCAGATCCTGAGAATGGTCCGAATGGACAGAAGAGGCGGCACCTGGAAGCTGCTGG
GCTCTGTGGTGTACGCCCACAGCAAAGAGCTGATCACCGCCTGGTACATCGGATTTCTGGTGCT
GATCTTCGCCTCCTTCCTGGTGTACCTGGCCGAGAAGGACGCCAACAGCGACTTTAGCAGCTAC
GCCGACTCTCTTTGGTGGGGCACCATCACACTGACCACCATCGGCTACGGCGACAAGACCCCTC
ACACATGGCTGGGAAGAGTGCTGGCCGCTGGATTTGCTCTGCTGGGCATCAGCTTTTTCGCCCT
GCCTGCCGGAATCCTCGGATCTGGCTTTGCCCTGAAGGTGCAAGAGCAGCATAGACAAAAGCAT
TTTGAAAAGAGAAGAATGCCTGCCGCCAACCTGATTCAGGCCGCTTGGAGACTGTACAGCACCG
ACATGAGCAGAGCCTACCTGACCGCCACGTGGTATTATTACGATTCGATCCTGCCTAGCTTCCG
CGAACTGGCCCTGCTGTTTGAGCATGTGCAGAGAGCCAGAAACGGCGGCCTCAGACCTCTGGAA
GTTCGGAGAGCACCTGTGCCTGATGGCGCCCCTTCTAGATATCCTCCAGTGGCCACCTGTCACA
GACCCGGCAGCACATCTTTTTGCCCTGGCGAGTCTAGCCGGATGGGCATCAAGGACAGAATCAG
AATGGGCAGCAGCCAGCGGAGAACAGGCCCTTCTAAACAGCATCTGGCCCCTCCAACCATGCCT
ACAAGCCCTAGCTCTGAGCAAGTGGGCGAAGCCACCTCTCCTACCAAGGTGCAGAAGTCCTGGT
CCTTCAACGACCGGACCAGATTTAGAGCCAGCCTCCGGCTGAAGCCCAGAACCTCTGCCGAGGA
TGCCCCTTCTGAAGAGGTGGCCGAAGAGAAGTCCTACCAGTGCGAGCTGACCGTGGACGACATC
ATGCCAGCCGTGAAAACCGTGATAAGGTCTATACGCATCCTGAAGTTCCTGGTGGCCAAGCGGA
AGTTCAAAGAGACACTGCGGCCCTACGACGTGAAGGACGTGATCGAGCAGTATTCTGCCGGCCA
CCTGGACATGCTGGGCAGAATCAAGAGCCTGCAGACCAGAGTGGACCAGATCGTTGGAAGAGGC
CCAGGCGACAGAAAGGCCAGAGAGAAGGGCGATAAGGGCCCATCTGATGCCGAGGTTGTCGACG
AGATATCAATGATGGGCAGAGTCGTGAAAGTCGAAAAACAGGTGCAGAGCATCGAGCACAAGCT
GGACCTGCTGCTGGGATTCTACAGCCGGTGTCTGAGAAGCGGCACATCTGCATCTCTGGGCGCT
GTGCAGGTCCCACTGTTCGATCCTGATATTACGAGCGATTATCACAGCCCCGTGGACCACGAGG
ACATCTCCGTTTCTGCTCAGACCCTGAGCATCAGCAGATCCGTGTCCACCAACATGGACTAAcc
tgcaggcgccggcgGAGCAGGAGGACCAAGGCCCTGGCGAAGGCCGTGGCCTCGATGCTGTCAT
AGTAATACCAGTGTGCAGGTCCCAATGGCTGGTATTAATGTGACAGTCTCGGGGACGCGGGCCT
GGACGCCGGCATCCGGGCTCAGGACCCCCCTCGAGCTCGCTGATCAGCCTCGACTGTGCCTTCT
AGTTGCCAGCCATCTGTTGTTTGCCCCTCCCCCGTGCCTTCCTTGACCCTGGAAGGTGCCACTC
CCACTGTCCTTTCCTAATAAAATGAGGAAATTGCATCGCATTGTCTGAGTAGGTGTCATTCTAT
TCTGGGGGGTGGGGTGGGGCAGGACAGCAAGGGGGAGGATTGGGAAGACAATAGCAGGCATGCT
GGGGATGCGGTGGGCTCTATGGAAGCTTGAATTCAGCTGACGTGCCTCGGACCGCTAGGAACCC
CTAGTGATGGAGTTGGCCACTCCCTCTCTGCGCGCTCGCTCGCTCACTGAGGCCGGGCGACCAA
AGGTCGCCCGACGCCCGGGCTTTGCCCGGGCGGCCTCAGTGAGCGAGCGAGCGCGCAG
Exemplary pITR.CAG.mmu.miR2i_miR6u-26_EGFP
(SEQ ID NO: 247)
CTGCGCGCTCGCTCGCTCACTGAGGCCGCCCGGGCGTCGGGCGACCTTTGGTCGCCCGGCCTCA
GTGAGCGAGCGAGCGCGCAGAGAGGGAGTGGCCAACTCCATCACTAGGGGTTCCTGCGGCCGCA
CGCGTGACATTGATTATTGACTAGTTATTAATAGTAATCAATTACGGGGTCATTAGTTCATAGC
CCATATATGGAGTTCCGCGTTACATAACTTACGGTAAATGGCCCGCCTGGCTGACCGCCCAACG
ACCCCCGCCCATTGACGTCAATAATGACGTATGTTCCCATAGTAACGCCAATAGGGACTTTCCA
TTGACGTCAATGGGTGGACTATTTACGGTAAACTGCCCACTTGGCAGTACATCAAGTGTATCAT
ATGCCAAGTACGCCCCCTATTGACGTCAATGACGGTAAATGGCCCGCCTGGCATTATGCCCAGT
ACATGACCTTATGGGACTTTCCTACTTGGCAGTACATCTACGTATTAGTCATCGCTATTACCAT
GGGTCGAGGTGAGCCCCACGTTCTGCTTCACTCTCCCCATCTCCCCCCCCTCCCCACCCCCAAT
TTTGTATTTATTTATTTTTTAATTATTTTGTGCAGCGATGGGGGCGGGGGGGGGGGGGGCGCGC
GCCAGGCGGGGCGGGGCGGGGCGAGGGGCGGGGCGGGGCGAGGCGGAGAGGTGCGGCGGCAGCC
AATCAGAGCGGCGCGCTCCGAAAGTTTCCTTTTATGGCGAGGCGGCGGCGGCGGCGGCCCTATA
AAAAGCGAAGCGCGCGGCGGGCGGGAGTCGCTGCGTTGCCTTCGCCCCGTGCCCCGCTCCGCGC
CGCCTCGCGCCGCCCGCCCCGGCTCTGACTGACCGCGTTACTCCCACAGGTGAGCGGGCGGGAC
GGCCCTTCTCCTCCGGGCTGTAATTAGCGCTTGGTTTAATGACGGCTCGTTTCTTTTCTGTGGC
TGCGTGAAAGCCTTAAAGGGCTCCGGGAGGGCCCTTTGTGCGGGGGGGAGCGGCTCGGGGGGTG
CGTGCGTGTGTGTGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGCGGCCGGGGGCGG
TGCCCCGCGGTGCGGGGGGGCTGCGAGGGGAACAAAGGCTGCGTGCGGGGTGTGTGCGTGGGGG
GGTGAGCAGGGGGTGTGGGCGCGGCGGTCGGGCTGTAACCCCCCCCTGCACCCCCCTCCCCGAG
TTGCTGAGCACGGCCCGGCTTCGGGTGCGGGGCTCCGTGCGGGGCGTGGCGCGGGGCTCGCCGT
GCCGGGCGGGGGGTGGCGGCAGGTGGGGGTGCCGGGCGGGGCGGGGCCGCCTCGGGCCGGGGAG
GGCTCGGGGGAGGGGCGCGGCGGCCCCCGGAGCGCCGGCGGAGCAGGAGGACCAAGGCCCTGGC
GAAGGCCGTGGCCTCGTTGAGTCTTAGAGAGGCCCGGTGTGCAGGTCCCAATGGCCGGGTCTTA
CTGAGATTCCACGGGGACGCGGGCCTGGACGCCGGCATCCGGGCTCAGGACCCCCCTCTCTAGA
GCCTCTGCTAACCATGTTCATGCCTTCTTCTTTTTCCTACAGCTCCTGGGCAACGTGCTGGTTA
TTGTGACCGGTCGCTAGCCACCATGGTGAGCAAGGGCGAGGAGCTGTTCACCGGGGTGGTGCCC
ATCCTGGTCGAGCTGGACGGCGACGTAAACGGCCACAAGTTCAGCGTGTCCGGCGAGGGCGAGG
GCGATGCCACCTACGGCAAGCTGACCCTGAAGTTCATCTGCACCACCGGCAAGCTGCCCGTGCC
CTGGCCCACCCTCGTGACCACCCTGACCTACGGCGTGCAGTGCTTCAGCCGCTACCCCGACCAC
ATGAAGCAGCACGACTTCTTCAAGTCCGCCATGCCCGAAGGCTACGTCCAGGAGCGCACCATCT
TCTTCAAGGACGACGGCAACTACAAGACCCGCGCCGAGGTGAAGTTCGAGGGCGACACCCTGGT
GAACCGCATCGAGCTGAAGGGCATCGACTTCAAGGAGGACGGCAACATCCTGGGGCACAAGCTG
GAGTACAACTACAACAGCCACAACGTCTATATCATGGCCGACAAGCAGAAGAACGGCATCAAGG
TGAACTTCAAGATCCGCCACAACATCGAGGACGGCAGCGTGCAGCTCGCCGACCACTACCAGCA
GAACACCCCCATCGGCGACGGCCCCGTGCTGCTGCCCGACAACCACTACCTGAGCACCCAGTCC
GCCCTGAGCAAAGACCCCAACGAGAAGCGCGATCACATGGTCCTGCTGGAGTTCGTGACCGCCG
CCGGGATCACTCTCGGCATGGACGAGCTGTACAAGTAAcctgcaggcgccggcgGAGCAGGAGG
ACCAAGGCCCTGGCGAAGGCCGTGGCCTCGATGCTGTCATAGTAATACCAGTGTGCAGGTCCCA
ATGGCTGGTATTAATGTGACAGTCTCGGGGACGCGGGCCTGGACGCCGGCATCCGGGCTCAGGA
CCCCCCTCGAGCTCGCTGATCAGCCTCGACTGTGCCTTCTAGTTGCCAGCCATCTGTTGTTTGC
CCCTCCCCCGTGCCTTCCTTGACCCTGGAAGGTGCCACTCCCACTGTCCTTTCCTAATAAAATG
AGGAAATTGCATCGCATTGTCTGAGTAGGTGTCATTCTATTCTGGGGGGTGGGGTGGGGCAGGA
CAGCAAGGGGGAGGATTGGGAAGACAATAGCAGGCATGCTGGGGATGCGGTGGGCTCTATGGAA
GCTTGAATTCAGCTGACGTGCCTCGGACCGCTAGGAACCCCTAGTGATGGAGTTGGCCACTCCC
TCTCTGCGCGCTCGCTCGCTCACTGAGGCCGGGCGACCAAAGGTCGCCCGACGCCCGGGCTTTG
CCCGGGCGGCCTCAGTGAGCGAGCGAGCGCGCAG
Exemplary pITR.CAG.mmu.miR2i_miR6u-26_KCNQ4codop
(SEQ ID NO: 248)
CTGCGCGCTCGCTCGCTCACTGAGGCCGCCCGGGCGTCGGGCGACCTTTGGTCGCCCGGCCTCA
GTGAGCGAGCGAGCGCGCAGAGAGGGAGTGGCCAACTCCATCACTAGGGGTTCCTGCGGCCGCA
CGCGTGACATTGATTATTGACTAGTTATTAATAGTAATCAATTACGGGGTCATTAGTTCATAGC
CCATATATGGAGTTCCGCGTTACATAACTTACGGTAAATGGCCCGCCTGGCTGACCGCCCAACG
ACCCCCGCCCATTGACGTCAATAATGACGTATGTTCCCATAGTAACGCCAATAGGGACTTTCCA
TTGACGTCAATGGGTGGACTATTTACGGTAAACTGCCCACTTGGCAGTACATCAAGTGTATCAT
ATGCCAAGTACGCCCCCTATTGACGTCAATGACGGTAAATGGCCCGCCTGGCATTATGCCCAGT
ACATGACCTTATGGGACTTTCCTACTTGGCAGTACATCTACGTATTAGTCATCGCTATTACCAT
GGGTCGAGGTGAGCCCCACGTTCTGCTTCACTCTCCCCATCTCCCCCCCCTCCCCACCCCCAAT
TTTGTATTTATTTATTTTTTAATTATTTTGTGCAGCGATGGGGGCGGGGGGGGGGGGGGCGCGC
GCCAGGCGGGGCGGGGCGGGGCGAGGGGCGGGGCGGGGCGAGGCGGAGAGGTGCGGCGGCAGCC
AATCAGAGCGGCGCGCTCCGAAAGTTTCCTTTTATGGCGAGGCGGCGGCGGCGGCGGCCCTATA
AAAAGCGAAGCGCGCGGCGGGCGGGAGTCGCTGCGTTGCCTTCGCCCCGTGCCCCGCTCCGCGC
CGCCTCGCGCCGCCCGCCCCGGCTCTGACTGACCGCGTTACTCCCACAGGTGAGCGGGCGGGAC
GGCCCTTCTCCTCCGGGCTGTAATTAGCGCTTGGTTTAATGACGGCTCGTTTCTTTTCTGTGGC
TGCGTGAAAGCCTTAAAGGGCTCCGGGAGGGCCCTTTGTGCGGGGGGGAGCGGCTCGGGGGGTG
CGTGCGTGTGTGTGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGCGGCCGGGGGCGG
TGCCCCGCGGTGCGGGGGGGCTGCGAGGGGAACAAAGGCTGCGTGCGGGGTGTGTGCGTGGGGG
GGTGAGCAGGGGGTGTGGGCGCGGCGGTCGGGCTGTAACCCCCCCCTGCACCCCCCTCCCCGAG
TTGCTGAGCACGGCCCGGCTTCGGGTGCGGGGCTCCGTGCGGGGCGTGGCGCGGGGCTCGCCGT
GCCGGGCGGGGGGTGGCGGCAGGTGGGGGTGCCGGGCGGGGCGGGGCCGCCTCGGGCCGGGGAG
GGCTCGGGGGAGGGGCGCGGCGGCCCCCGGAGCGCCGGCGGAGCAGGAGGACCAAGGCCCTGGC
GAAGGCCGTGGCCTCGTTGAGTCTTAGAGAGGCCCGGTGTGCAGGTCCCAATGGCCGGGTCTTA
CTGAGATTCCACGGGGACGCGGGCCTGGACGCCGGCATCCGGGCTCAGGACCCCCCTCTCTAGA
GCCTCTGCTAACCATGTTCATGCCTTCTTCTTTTTCCTACAGCTCCTGGGCAACGTGCTGGTTA
TTGTGACCGGTCGCTAGCCACCATGGCTGAAGCCCCTCCTAGAAGGCTTGGACTGGGACCTCCT
CCTGGGGATGCTCCTAGAGCTGAACTGGTGGCTCTGACAGCCGTGCAGTCTGAACAAGGCGAAG
CTGGTGGCGGCGGATCTCCACGTAGACTTGGACTGCTGGGAAGCCCTCTTCCTCCTGGTGCTCC
ACTTCCTGGACCTGGCAGTGGATCTGGATCTGCCTGTGGCCAGAGAAGCTCTGCCGCTCACAAG
AGATACCGGCGGCTGCAGAACTGGGTGTACAACGTGCTGGAAAGACCCAGAGGCTGGGCCTTCG
TGTACCACGTGTTCATCTTTCTGCTGGTGTTCAGCTGCCTGGTGCTGTCCGTGCTGAGCACCAT
CCAAGAACATCAAGAGCTGGCTAACGAGTGCCTGTTAATACTGGAGTTTGTGATGATTGTGGTG
TTCGGCCTCGAGTACATCGTCCGCGTTTGGAGCGCCGGCTGCTGCTGCAGATATAGAGGTTGGC
AAGGCAGATTCCGCTTCGCCAGAAAGCCCTTCTGCGTGATCGACTTCATCGTGTTCGTGGCCAG
CGTGGCCGTGATTGCTGCTGGCACACAGGGCAACATCTTCGCCACAAGCGCCCTGCGGAGCATG
CGGTTTCTGCAGATCCTGAGAATGGTCCGAATGGACAGAAGAGGCGGCACCTGGAAGCTGCTGG
GCTCTGTGGTGTACGCCCACAGCAAAGAGCTGATCACCGCCTGGTACATCGGATTTCTGGTGCT
GATCTTCGCCTCCTTCCTGGTGTACCTGGCCGAGAAGGACGCCAACAGCGACTTTAGCAGCTAC
GCCGACTCTCTTTGGTGGGGCACCATCACACTGACCACCATCGGCTACGGCGACAAGACCCCTC
ACACATGGCTGGGAAGAGTGCTGGCCGCTGGATTTGCTCTGCTGGGCATCAGCTTTTTCGCCCT
GCCTGCCGGAATCCTCGGATCTGGCTTTGCCCTGAAGGTGCAAGAGCAGCATAGACAAAAGCAT
TTTGAAAAGAGAAGAATGCCTGCCGCCAACCTGATTCAGGCCGCTTGGAGACTGTACAGCACCG
ACATGAGCAGAGCCTACCTGACCGCCACGTGGTATTATTACGATTCGATCCTGCCTAGCTTCCG
CGAACTGGCCCTGCTGTTTGAGCATGTGCAGAGAGCCAGAAACGGCGGCCTCAGACCTCTGGAA
GTTCGGAGAGCACCTGTGCCTGATGGCGCCCCTTCTAGATATCCTCCAGTGGCCACCTGTCACA
GACCCGGCAGCACATCTTTTTGCCCTGGCGAGTCTAGCCGGATGGGCATCAAGGACAGAATCAG
AATGGGCAGCAGCCAGCGGAGAACAGGCCCTTCTAAACAGCATCTGGCCCCTCCAACCATGCCT
ACAAGCCCTAGCTCTGAGCAAGTGGGCGAAGCCACCTCTCCTACCAAGGTGCAGAAGTCCTGGT
CCTTCAACGACCGGACCAGATTTAGAGCCAGCCTCCGGCTGAAGCCCAGAACCTCTGCCGAGGA
TGCCCCTTCTGAAGAGGTGGCCGAAGAGAAGTCCTACCAGTGCGAGCTGACCGTGGACGACATC
ATGCCAGCCGTGAAAACCGTGATAAGGTCTATACGCATCCTGAAGTTCCTGGTGGCCAAGCGGA
AGTTCAAAGAGACACTGCGGCCCTACGACGTGAAGGACGTGATCGAGCAGTATTCTGCCGGCCA
CCTGGACATGCTGGGCAGAATCAAGAGCCTGCAGACCAGAGTGGACCAGATCGTTGGAAGAGGC
CCAGGCGACAGAAAGGCCAGAGAGAAGGGCGATAAGGGCCCATCTGATGCCGAGGTTGTCGACG
AGATATCAATGATGGGCAGAGTCGTGAAAGTCGAAAAACAGGTGCAGAGCATCGAGCACAAGCT
GGACCTGCTGCTGGGATTCTACAGCCGGTGTCTGAGAAGCGGCACATCTGCATCTCTGGGCGCT
GTGCAGGTCCCACTGTTCGATCCTGATATTACGAGCGATTATCACAGCCCCGTGGACCACGAGG
ACATCTCCGTTTCTGCTCAGACCCTGAGCATCAGCAGATCCGTGTCCACCAACATGGACTAAcc
tgcaggcgccggcgGAGCAGGAGGACCAAGGCCCTGGCGAAGGCCGTGGCCTCGATGCTGTCAT
AGTAATACCAGTGTGCAGGTCCCAATGGCTGGTATTAATGTGACAGTCTCGGGGACGCGGGCCT
GGACGCCGGCATCCGGGCTCAGGACCCCCCTCGAGCTCGCTGATCAGCCTCGACTGTGCCTTCT
AGTTGCCAGCCATCTGTTGTTTGCCCCTCCCCCGTGCCTTCCTTGACCCTGGAAGGTGCCACTC
CCACTGTCCTTTCCTAATAAAATGAGGAAATTGCATCGCATTGTCTGAGTAGGTGTCATTCTAT
TCTGGGGGGTGGGGTGGGGCAGGACAGCAAGGGGGAGGATTGGGAAGACAATAGCAGGCATGCT
GGGGATGCGGTGGGCTCTATGGAAGCTTGAATTCAGCTGACGTGCCTCGGACCGCTAGGAACCC
CTAGTGATGGAGTTGGCCACTCCCTCTCTGCGCGCTCGCTCGCTCACTGAGGCCGGGCGACCAA
AGGTCGCCCGACGCCCGGGCTTTGCCCGGGCGGCCTCAGTGAGCGAGCGAGCGCGCAG
Exemplary pITR.CAG.mmu.miR5i_miR6u-26_EGFP
(SEQ ID NO: 249)
CTGCGCGCTCGCTCGCTCACTGAGGCCGCCCGGGCGTCGGGCGACCTTTGGTCGCCCGGCCTCA
GTGAGCGAGCGAGCGCGCAGAGAGGGAGTGGCCAACTCCATCACTAGGGGTTCCTGCGGCCGCA
CGCGTGACATTGATTATTGACTAGTTATTAATAGTAATCAATTACGGGGTCATTAGTTCATAGC
CCATATATGGAGTTCCGCGTTACATAACTTACGGTAAATGGCCCGCCTGGCTGACCGCCCAACG
ACCCCCGCCCATTGACGTCAATAATGACGTATGTTCCCATAGTAACGCCAATAGGGACTTTCCA
TTGACGTCAATGGGTGGACTATTTACGGTAAACTGCCCACTTGGCAGTACATCAAGTGTATCAT
ATGCCAAGTACGCCCCCTATTGACGTCAATGACGGTAAATGGCCCGCCTGGCATTATGCCCAGT
ACATGACCTTATGGGACTTTCCTACTTGGCAGTACATCTACGTATTAGTCATCGCTATTACCAT
GGGTCGAGGTGAGCCCCACGTTCTGCTTCACTCTCCCCATCTCCCCCCCCTCCCCACCCCCAAT
TTTGTATTTATTTATTTTTTAATTATTTTGTGCAGCGATGGGGGCGGGGGGGGGGGGGGCGCGC
GCCAGGCGGGGCGGGGCGGGGCGAGGGGCGGGGCGGGGCGAGGCGGAGAGGTGCGGCGGCAGCC
AATCAGAGCGGCGCGCTCCGAAAGTTTCCTTTTATGGCGAGGCGGCGGCGGCGGCGGCCCTATA
AAAAGCGAAGCGCGCGGCGGGCGGGAGTCGCTGCGTTGCCTTCGCCCCGTGCCCCGCTCCGCGC
CGCCTCGCGCCGCCCGCCCCGGCTCTGACTGACCGCGTTACTCCCACAGGTGAGCGGGCGGGAC
GGCCCTTCTCCTCCGGGCTGTAATTAGCGCTTGGTTTAATGACGGCTCGTTTCTTTTCTGTGGC
TGCGTGAAAGCCTTAAAGGGCTCCGGGAGGGCCCTTTGTGCGGGGGGGAGCGGCTCGGGGGGTG
CGTGCGTGTGTGTGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGCGGCCGGGGGCGG
TGCCCCGCGGTGCGGGGGGGCTGCGAGGGGAACAAAGGCTGCGTGCGGGGTGTGTGCGTGGGGG
GGTGAGCAGGGGGTGTGGGCGCGGCGGTCGGGCTGTAACCCCCCCCTGCACCCCCCTCCCCGAG
TTGCTGAGCACGGCCCGGCTTCGGGTGCGGGGCTCCGTGCGGGGCGTGGCGCGGGGCTCGCCGT
GCCGGGCGGGGGGTGGCGGCAGGTGGGGGTGCCGGGCGGGGCGGGGCCGCCTCGGGCCGGGGAG
GGCTCGGGGGAGGGGCGCGGCGGCCCCCGGAGCGCCGGCGGAGCAGGAGGACCAAGGCCCTGGC
GAAGGCCGTGGCCTCGTTTTCCACCTTGACTACGCGCTGTGCAGGTCCCAATGGGCGTGTAGTA
GAGGTGGAGCACGGGGACGCGGGCCTGGACGCCGGCATCCGGGCTCAGGACCCCCCTCTCTAGA
GCCTCTGCTAACCATGTTCATGCCTTCTTCTTTTTCCTACAGCTCCTGGGCAACGTGCTGGTTA
TTGTGACCGGTCGCTAGCCACCATGGTGAGCAAGGGCGAGGAGCTGTTCACCGGGGTGGTGCCC
ATCCTGGTCGAGCTGGACGGCGACGTAAACGGCCACAAGTTCAGCGTGTCCGGCGAGGGCGAGG
GCGATGCCACCTACGGCAAGCTGACCCTGAAGTTCATCTGCACCACCGGCAAGCTGCCCGTGCC
CTGGCCCACCCTCGTGACCACCCTGACCTACGGCGTGCAGTGCTTCAGCCGCTACCCCGACCAC
ATGAAGCAGCACGACTTCTTCAAGTCCGCCATGCCCGAAGGCTACGTCCAGGAGCGCACCATCT
TCTTCAAGGACGACGGCAACTACAAGACCCGCGCCGAGGTGAAGTTCGAGGGCGACACCCTGGT
GAACCGCATCGAGCTGAAGGGCATCGACTTCAAGGAGGACGGCAACATCCTGGGGCACAAGCTG
GAGTACAACTACAACAGCCACAACGTCTATATCATGGCCGACAAGCAGAAGAACGGCATCAAGG
TGAACTTCAAGATCCGCCACAACATCGAGGACGGCAGCGTGCAGCTCGCCGACCACTACCAGCA
GAACACCCCCATCGGCGACGGCCCCGTGCTGCTGCCCGACAACCACTACCTGAGCACCCAGTCC
GCCCTGAGCAAAGACCCCAACGAGAAGCGCGATCACATGGTCCTGCTGGAGTTCGTGACCGCCG
CCGGGATCACTCTCGGCATGGACGAGCTGTACAAGTAAcctgcaggcgccggcgGAGCAGGAGG
ACCAAGGCCCTGGCGAAGGCCGTGGCCTCGATGCTGTCATAGTAATACCAGTGTGCAGGTCCCA
ATGGCTGGTATTAATGTGACAGTCTCGGGGACGCGGGCCTGGACGCCGGCATCCGGGCTCAGGA
CCCCCCTCGAGCTCGCTGATCAGCCTCGACTGTGCCTTCTAGTTGCCAGCCATCTGTTGTTTGC
CCCTCCCCCGTGCCTTCCTTGACCCTGGAAGGTGCCACTCCCACTGTCCTTTCCTAATAAAATG
AGGAAATTGCATCGCATTGTCTGAGTAGGTGTCATTCTATTCTGGGGGGTGGGGTGGGGCAGGA
CAGCAAGGGGGAGGATTGGGAAGACAATAGCAGGCATGCTGGGGATGCGGTGGGCTCTATGGAA
GCTTGAATTCAGCTGACGTGCCTCGGACCGCTAGGAACCCCTAGTGATGGAGTTGGCCACTCCC
TCTCTGCGCGCTCGCTCGCTCACTGAGGCCGGGCGACCAAAGGTCGCCCGACGCCCGGGCTTTG
CCCGGGCGGCCTCAGTGAGCGAGCGAGCGCGCAG
Exemplary pITR.CAG.mmu.miR5i_miR6u-26_KCNQ4codop
(SEQ ID NO: 250)
CTGCGCGCTCGCTCGCTCACTGAGGCCGCCCGGGCGTCGGGCGACCTTTGGTCGCCCGGCCTCA
GTGAGCGAGCGAGCGCGCAGAGAGGGAGTGGCCAACTCCATCACTAGGGGTTCCTGCGGCCGCA
CGCGTGACATTGATTATTGACTAGTTATTAATAGTAATCAATTACGGGGTCATTAGTTCATAGC
CCATATATGGAGTTCCGCGTTACATAACTTACGGTAAATGGCCCGCCTGGCTGACCGCCCAACG
ACCCCCGCCCATTGACGTCAATAATGACGTATGTTCCCATAGTAACGCCAATAGGGACTTTCCA
TTGACGTCAATGGGTGGACTATTTACGGTAAACTGCCCACTTGGCAGTACATCAAGTGTATCAT
ATGCCAAGTACGCCCCCTATTGACGTCAATGACGGTAAATGGCCCGCCTGGCATTATGCCCAGT
ACATGACCTTATGGGACTTTCCTACTTGGCAGTACATCTACGTATTAGTCATCGCTATTACCAT
GGGTCGAGGTGAGCCCCACGTTCTGCTTCACTCTCCCCATCTCCCCCCCCTCCCCACCCCCAAT
TTTGTATTTATTTATTTTTTAATTATTTTGTGCAGCGATGGGGGCGGGGGGGGGGGGGGCGCGC
GCCAGGCGGGGCGGGGCGGGGCGAGGGGCGGGGCGGGGCGAGGCGGAGAGGTGCGGCGGCAGCC
AATCAGAGCGGCGCGCTCCGAAAGTTTCCTTTTATGGCGAGGCGGCGGCGGCGGCGGCCCTATA
AAAAGCGAAGCGCGCGGCGGGCGGGAGTCGCTGCGTTGCCTTCGCCCCGTGCCCCGCTCCGCGC
CGCCTCGCGCCGCCCGCCCCGGCTCTGACTGACCGCGTTACTCCCACAGGTGAGCGGGCGGGAC
GGCCCTTCTCCTCCGGGCTGTAATTAGCGCTTGGTTTAATGACGGCTCGTTTCTTTTCTGTGGC
TGCGTGAAAGCCTTAAAGGGCTCCGGGAGGGCCCTTTGTGCGGGGGGGAGCGGCTCGGGGGGTG
CGTGCGTGTGTGTGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGCGGCCGGGGGCGG
TGCCCCGCGGTGCGGGGGGGCTGCGAGGGGAACAAAGGCTGCGTGCGGGGTGTGTGCGTGGGGG
GGTGAGCAGGGGGTGTGGGCGCGGCGGTCGGGCTGTAACCCCCCCCTGCACCCCCCTCCCCGAG
TTGCTGAGCACGGCCCGGCTTCGGGTGCGGGGCTCCGTGCGGGGCGTGGCGCGGGGCTCGCCGT
GCCGGGCGGGGGGTGGCGGCAGGTGGGGGTGCCGGGCGGGGCGGGGCCGCCTCGGGCCGGGGAG
GGCTCGGGGGAGGGGCGCGGCGGCCCCCGGAGCGCCGGCGGAGCAGGAGGACCAAGGCCCTGGC
GAAGGCCGTGGCCTCGTTTTCCACCTTGACTACGCGCTGTGCAGGTCCCAATGGGCGTGTAGTA
GAGGTGGAGCACGGGGACGCGGGCCTGGACGCCGGCATCCGGGCTCAGGACCCCCCTCTCTAGA
GCCTCTGCTAACCATGTTCATGCCTTCTTCTTTTTCCTACAGCTCCTGGGCAACGTGCTGGTTA
TTGTGACCGGTCGCTAGCCACCATGGCTGAAGCCCCTCCTAGAAGGCTTGGACTGGGACCTCCT
CCTGGGGATGCTCCTAGAGCTGAACTGGTGGCTCTGACAGCCGTGCAGTCTGAACAAGGCGAAG
CTGGTGGCGGCGGATCTCCACGTAGACTTGGACTGCTGGGAAGCCCTCTTCCTCCTGGTGCTCC
ACTTCCTGGACCTGGCAGTGGATCTGGATCTGCCTGTGGCCAGAGAAGCTCTGCCGCTCACAAG
AGATACCGGCGGCTGCAGAACTGGGTGTACAACGTGCTGGAAAGACCCAGAGGCTGGGCCTTCG
TGTACCACGTGTTCATCTTTCTGCTGGTGTTCAGCTGCCTGGTGCTGTCCGTGCTGAGCACCAT
CCAAGAACATCAAGAGCTGGCTAACGAGTGCCTGTTAATACTGGAGTTTGTGATGATTGTGGTG
TTCGGCCTCGAGTACATCGTCCGCGTTTGGAGCGCCGGCTGCTGCTGCAGATATAGAGGTTGGC
AAGGCAGATTCCGCTTCGCCAGAAAGCCCTTCTGCGTGATCGACTTCATCGTGTTCGTGGCCAG
CGTGGCCGTGATTGCTGCTGGCACACAGGGCAACATCTTCGCCACAAGCGCCCTGCGGAGCATG
CGGTTTCTGCAGATCCTGAGAATGGTCCGAATGGACAGAAGAGGCGGCACCTGGAAGCTGCTGG
GCTCTGTGGTGTACGCCCACAGCAAAGAGCTGATCACCGCCTGGTACATCGGATTTCTGGTGCT
GATCTTCGCCTCCTTCCTGGTGTACCTGGCCGAGAAGGACGCCAACAGCGACTTTAGCAGCTAC
GCCGACTCTCTTTGGTGGGGCACCATCACACTGACCACCATCGGCTACGGCGACAAGACCCCTC
ACACATGGCTGGGAAGAGTGCTGGCCGCTGGATTTGCTCTGCTGGGCATCAGCTTTTTCGCCCT
GCCTGCCGGAATCCTCGGATCTGGCTTTGCCCTGAAGGTGCAAGAGCAGCATAGACAAAAGCAT
TTTGAAAAGAGAAGAATGCCTGCCGCCAACCTGATTCAGGCCGCTTGGAGACTGTACAGCACCG
ACATGAGCAGAGCCTACCTGACCGCCACGTGGTATTATTACGATTCGATCCTGCCTAGCTTCCG
CGAACTGGCCCTGCTGTTTGAGCATGTGCAGAGAGCCAGAAACGGCGGCCTCAGACCTCTGGAA
GTTCGGAGAGCACCTGTGCCTGATGGCGCCCCTTCTAGATATCCTCCAGTGGCCACCTGTCACA
GACCCGGCAGCACATCTTTTTGCCCTGGCGAGTCTAGCCGGATGGGCATCAAGGACAGAATCAG
AATGGGCAGCAGCCAGCGGAGAACAGGCCCTTCTAAACAGCATCTGGCCCCTCCAACCATGCCT
ACAAGCCCTAGCTCTGAGCAAGTGGGCGAAGCCACCTCTCCTACCAAGGTGCAGAAGTCCTGGT
CCTTCAACGACCGGACCAGATTTAGAGCCAGCCTCCGGCTGAAGCCCAGAACCTCTGCCGAGGA
TGCCCCTTCTGAAGAGGTGGCCGAAGAGAAGTCCTACCAGTGCGAGCTGACCGTGGACGACATC
ATGCCAGCCGTGAAAACCGTGATAAGGTCTATACGCATCCTGAAGTTCCTGGTGGCCAAGCGGA
AGTTCAAAGAGACACTGCGGCCCTACGACGTGAAGGACGTGATCGAGCAGTATTCTGCCGGCCA
CCTGGACATGCTGGGCAGAATCAAGAGCCTGCAGACCAGAGTGGACCAGATCGTTGGAAGAGGC
CCAGGCGACAGAAAGGCCAGAGAGAAGGGCGATAAGGGCCCATCTGATGCCGAGGTTGTCGACG
AGATATCAATGATGGGCAGAGTCGTGAAAGTCGAAAAACAGGTGCAGAGCATCGAGCACAAGCT
GGACCTGCTGCTGGGATTCTACAGCCGGTGTCTGAGAAGCGGCACATCTGCATCTCTGGGCGCT
GTGCAGGTCCCACTGTTCGATCCTGATATTACGAGCGATTATCACAGCCCCGTGGACCACGAGG
ACATCTCCGTTTCTGCTCAGACCCTGAGCATCAGCAGATCCGTGTCCACCAACATGGACTAAcc
tgcaggcgccggcgGAGCAGGAGGACCAAGGCCCTGGCGAAGGCCGTGGCCTCGATGCTGTCAT
AGTAATACCAGTGTGCAGGTCCCAATGGCTGGTATTAATGTGACAGTCTCGGGGACGCGGGCCT
GGACGCCGGCATCCGGGCTCAGGACCCCCCTCGAGCTCGCTGATCAGCCTCGACTGTGCCTTCT
AGTTGCCAGCCATCTGTTGTTTGCCCCTCCCCCGTGCCTTCCTTGACCCTGGAAGGTGCCACTC
CCACTGTCCTTTCCTAATAAAATGAGGAAATTGCATCGCATTGTCTGAGTAGGTGTCATTCTAT
TCTGGGGGGTGGGGTGGGGCAGGACAGCAAGGGGGAGGATTGGGAAGACAATAGCAGGCATGCT
GGGGATGCGGTGGGCTCTATGGAAGCTTGAATTCAGCTGACGTGCCTCGGACCGCTAGGAACCC
CTAGTGATGGAGTTGGCCACTCCCTCTCTGCGCGCTCGCTCGCTCACTGAGGCCGGGCGACCAA
AGGTCGCCCGACGCCCGGGCTTTGCCCGGGCGGCCTCAGTGAGCGAGCGAGCGCGCAG
Exemplary pITR-CAG.mmu.1xmiR2i_3xmiR6u-26_EGFP
(SEQ ID NO: 251)
CTGCGCGCTCGCTCGCTCACTGAGGCCGCCCGGGCGTCGGGCGACCTTTGGTCGCCCGGCCTCA
GTGAGCGAGCGAGCGCGCAGAGAGGGAGTGGCCAACTCCATCACTAGGGGTTCCTGCGGCCGCA
CGCGTGACATTGATTATTGACTAGTTATTAATAGTAATCAATTACGGGGTCATTAGTTCATAGC
CCATATATGGAGTTCCGCGTTACATAACTTACGGTAAATGGCCCGCCTGGCTGACCGCCCAACG
ACCCCCGCCCATTGACGTCAATAATGACGTATGTTCCCATAGTAACGCCAATAGGGACTTTCCA
TTGACGTCAATGGGTGGACTATTTACGGTAAACTGCCCACTTGGCAGTACATCAAGTGTATCAT
ATGCCAAGTACGCCCCCTATTGACGTCAATGACGGTAAATGGCCCGCCTGGCATTATGCCCAGT
ACATGACCTTATGGGACTTTCCTACTTGGCAGTACATCTACGTATTAGTCATCGCTATTACCAT
GGGTCGAGGTGAGCCCCACGTTCTGCTTCACTCTCCCCATCTCCCCCCCCTCCCCACCCCCAAT
TTTGTATTTATTTATTTTTTAATTATTTTGTGCAGCGATGGGGGCGGGGGGGGGGGGGGCGCGC
GCCAGGCGGGGCGGGGCGGGGCGAGGGGCGGGGCGGGGCGAGGCGGAGAGGTGCGGCGGCAGCC
AATCAGAGCGGCGCGCTCCGAAAGTTTCCTTTTATGGCGAGGCGGCGGCGGCGGCGGCCCTATA
AAAAGCGAAGCGCGCGGCGGGCGGGAGTCGCTGCGTTGCCTTCGCCCCGTGCCCCGCTCCGCGC
CGCCTCGCGCCGCCCGCCCCGGCTCTGACTGACCGCGTTACTCCCACAGGTGAGCGGGCGGGAC
GGCCCTTCTCCTCCGGGCTGTAATTAGCGCTTGGTTTAATGACGGCTCGTTTCTTTTCTGTGGC
TGCGTGAAAGCCTTAAAGGGCTCCGGGAGGGCCCTTTGTGCGGGGGGGAGCGGCTCGGGGGGTG
CGTGCGTGTGTGTGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGCGGCCGGGGGCGG
TGCCCCGCGGTGCGGGGGGGCTGCGAGGGGAACAAAGGCTGCGTGCGGGGTGTGTGCGTGGGGG
GGTGAGCAGGGGGTGTGGGCGCGGCGGTCGGGCTGTAACCCCCCCCTGCACCCCCCTCCCCGAG
TTGCTGAGCACGGCCCGGCTTCGGGTGCGGGGCTCCGTGCGGGGCGTGGCGCGGGGCTCGCCGT
GCCGGGCGGGGGGTGGCGGCAGGTGGGGGTGCCGGGCGGGGCGGGGCCGCCTCGGGCCGGGGAG
GGCTCGGGGGAGGGGCGCGGCGGCCCCCGGAGCGCCGGCGGAGCAGGAGGACCAAGGCCCTGGC
GAAGGCCGTGGCCTCGTTGAGTCTTAGAGAGGCCCGGTGTGCAGGTCCCAATGGCCGGGTCTTA
CTGAGATTCCACGGGGACGCGGGCCTGGACGCCGGCATCCGGGCTCAGGACCCCCCTCTCTAGA
GCCTCTGCTAACCATGTTCATGCCTTCTTCTTTTTCCTACAGCTCCTGGGCAACGTGCTGGTTA
TTGTGACCGGTCGCTAGCCACCATGGTGAGCAAGGGCGAGGAGCTGTTCACCGGGGTGGTGCCC
ATCCTGGTCGAGCTGGACGGCGACGTAAACGGCCACAAGTTCAGCGTGTCCGGCGAGGGCGAGG
GCGATGCCACCTACGGCAAGCTGACCCTGAAGTTCATCTGCACCACCGGCAAGCTGCCCGTGCC
CTGGCCCACCCTCGTGACCACCCTGACCTACGGCGTGCAGTGCTTCAGCCGCTACCCCGACCAC
ATGAAGCAGCACGACTTCTTCAAGTCCGCCATGCCCGAAGGCTACGTCCAGGAGCGCACCATCT
TCTTCAAGGACGACGGCAACTACAAGACCCGCGCCGAGGTGAAGTTCGAGGGCGACACCCTGGT
GAACCGCATCGAGCTGAAGGGCATCGACTTCAAGGAGGACGGCAACATCCTGGGGCACAAGCTG
GAGTACAACTACAACAGCCACAACGTCTATATCATGGCCGACAAGCAGAAGAACGGCATCAAGG
TGAACTTCAAGATCCGCCACAACATCGAGGACGGCAGCGTGCAGCTCGCCGACCACTACCAGCA
GAACACCCCCATCGGCGACGGCCCCGTGCTGCTGCCCGACAACCACTACCTGAGCACCCAGTCC
GCCCTGAGCAAAGACCCCAACGAGAAGCGCGATCACATGGTCCTGCTGGAGTTCGTGACCGCCG
CCGGGATCACTCTCGGCATGGACGAGCTGTACAAGTAAcctgcaggcgccggcgGAGCAGGAGG
ACCAAGGCCCTGGCGAAGGCCGTGGCCTCGATGCTGTCATAGTAATACCAGTGTGCAGGTCCCA
ATGGCTGGTATTAATGTGACAGTCTCGGGGACGCGCAATTGGGTGGCCTCGATGCTGTCATAGT
AATACCAGTGTGCAGGTCCCAATGGCTGGTATTAATGTGACAGTCTCGGGGACGCGGGCGCGCC
CGTGGCCTCGATGCTGTCATAGTAATACCAGTGTGCAGGTCCCAATGGCTGGTATTAATGTGAC
AGTCTCGGGGACGCGGGCCTGGACGCCGGCATCCGGGCTCAGGACCCCCCTCGAGCTCGCTGAT
CAGCCTCGACTGTGCCTTCTAGTTGCCAGCCATCTGTTGTTTGCCCCTCCCCCGTGCCTTCCTT
GACCCTGGAAGGTGCCACTCCCACTGTCCTTTCCTAATAAAATGAGGAAATTGCATCGCATTGT
CTGAGTAGGTGTCATTCTATTCTGGGGGGTGGGGTGGGGCAGGACAGCAAGGGGGAGGATTGGG
AAGACAATAGCAGGCATGCTGGGGATGCGGTGGGCTCTATGGAAGCTTGAATTCAGCTGACGTG
CCTCGGACCGCTAGGAACCCCTAGTGATGGAGTTGGCCACTCCCTCTCTGCGCGCTCGCTCGCT
CACTGAGGCCGGGCGACCAAAGGTCGCCCGACGCCCGGGCTTTGCCCGGGCGGCCTCAGTGAGC
GAGCGAGCGCGCAGCTGCCTGCAGG
Exemplary pITR-CAG.mmu.3xmiR2i_3xmiR6u-26_EGFP
(SEQ ID NO: 252)
CCTGCAGGCAGCTGCGCGCTCGCTCGCTCACTGAGGCCGCCCGGGCGTCGGGCGACCTTTGGTC
GCCCGGCCTCAGTGAGCGAGCGAGCGCGCAGAGAGGGAGTGGCCAACTCCATCACTAGGGGTTC
CTGCGGCCGCACGCGTGACATTGATTATTGACTAGTTATTAATAGTAATCAATTACGGGGTCAT
TAGTTCATAGCCCATATATGGAGTTCCGCGTTACATAACTTACGGTAAATGGCCCGCCTGGCTG
ACCGCCCAACGACCCCCGCCCATTGACGTCAATAATGACGTATGTTCCCATAGTAACGCCAATA
GGGACTTTCCATTGACGTCAATGGGTGGACTATTTACGGTAAACTGCCCACTTGGCAGTACATC
AAGTGTATCATATGCCAAGTACGCCCCCTATTGACGTCAATGACGGTAAATGGCCCGCCTGGCA
TTATGCCCAGTACATGACCTTATGGGACTTTCCTACTTGGCAGTACATCTACGTATTAGTCATC
GCTATTACCATGGGTCGAGGTGAGCCCCACGTTCTGCTTCACTCTCCCCATCTCCCCCCCCTCC
CCACCCCCAATTTTGTATTTATTTATTTTTTAATTATTTTGTGCAGCGATGGGGGCGGGGGGGG
GGGGGGCGCGCGCCAGGCGGGGCGGGGCGGGGCGAGGGGCGGGGCGGGGCGAGGCGGAGAGGTG
CGGCGGCAGCCAATCAGAGCGGCGCGCTCCGAAAGTTTCCTTTTATGGCGAGGCGGCGGCGGCG
GCGGCCCTATAAAAAGCGAAGCGCGCGGCGGGCGGGAGTCGCTGCGTTGCCTTCGCCCCGTGCC
CCGCTCCGCGCCGCCTCGCGCCGCCCGCCCCGGCTCTGACTGACCGCGTTACTCCCACAGGTGA
GCGGGCGGGACGGCCCTTCTCCTCCGGGCTGTAATTAGCGCTTGGTTTAATGACGGCTCGTTTC
TTTTCTGTGGCTGCGTGAAAGCCTTAAAGGGCTCCGGGAGGGCCCTTTGTGCGGGGGGGAGCGG
CTCGGGGGGTGCGTGCGTGTGTGTGTGCGTGGGGAGCGCCGCGTGCGGCCCGCGCTGCCCGGCG
GCTGTGAGCGCTGCGGGCGCGGCGCGGGGCTTTGTGCGCTCCGCGTGTGCGCGAGGGGAGCGCG
GCCGGGGGCGGTGCCCCGCGGTGCGGGGGGGCTGCGAGGGGAACAAAGGCTGCGTGCGGGGTGT
GTGCGTGGGGGGGTGAGCAGGGGGTGTGGGCGCGGCGGTCGGGCTGTAACCCCCCCCTGCACCC
CCCTCCCCGAGTTGCTGAGCACGGCCCGGCTTCGGGTGCGGGGCTCCGTGCGGGGCGTGGCGCG
GGGCTCGCCGTGCCGGGCGGGGGGTGGCGGCAGGTGGGGGTGCCGGGCGGGGCGGGGCCGCCTC
GGGCCGGGGAGGGCTCGGGGGAGGGGCGCGGCGGCCCCCGGAGCGCCGGCGGAGCAGGAGGACC
AAGGCCCTGGCGAAGGCCGTGGCCTCGTTGAGTCTTAGAGAGGCCCGGTGTGCAGGTCCCAATG
GCCGGGTCTTACTGAGATTCCACGGGGACGCGGGATCCGGTGGCCTCGTTGAGTCTTAGAGAGG
CCCGGTGTGCAGGTCCCAATGGCCGGGTCTTACTGAGATTCCACGGGGACGCGGGTACCCGTGG
CCTCGTTGAGTCTTAGAGAGGCCCGGTGTGCAGGTCCCAATGGCCGGGTCTTACTGAGATTCCA
CGGGGACGCGGGCCTGGACGCCGGCATCCGGGCTCAGGACCCCCCTCTCTAGAGCCTCTGCTAA
CCATGTTCATGCCTTCTTCTTTTTCCTACAGCTCCTGGGCAACGTGCTGGTTATTGTGACCGGT
CGCTAGCCACCATGGTGAGCAAGGGCGAGGAGCTGTTCACCGGGGTGGTGCCCATCCTGGTCGA
GCTGGACGGCGACGTAAACGGCCACAAGTTCAGCGTGTCCGGCGAGGGCGAGGGCGATGCCACC
TACGGCAAGCTGACCCTGAAGTTCATCTGCACCACCGGCAAGCTGCCCGTGCCCTGGCCCACCC
TCGTGACCACCCTGACCTACGGCGTGCAGTGCTTCAGCCGCTACCCCGACCACATGAAGCAGCA
CGACTTCTTCAAGTCCGCCATGCCCGAAGGCTACGTCCAGGAGCGCACCATCTTCTTCAAGGAC
GACGGCAACTACAAGACCCGCGCCGAGGTGAAGTTCGAGGGCGACACCCTGGTGAACCGCATCG
AGCTGAAGGGCATCGACTTCAAGGAGGACGGCAACATCCTGGGGCACAAGCTGGAGTACAACTA
CAACAGCCACAACGTCTATATCATGGCCGACAAGCAGAAGAACGGCATCAAGGTGAACTTCAAG
ATCCGCCACAACATCGAGGACGGCAGCGTGCAGCTCGCCGACCACTACCAGCAGAACACCCCCA
TCGGCGACGGCCCCGTGCTGCTGCCCGACAACCACTACCTGAGCACCCAGTCCGCCCTGAGCAA
AGACCCCAACGAGAAGCGCGATCACATGGTCCTGCTGGAGTTCGTGACCGCCGCCGGGATCACT
CTCGGCATGGACGAGCTGTACAAGTAAcctgcaggcgccggcgGAGCAGGAGGACCAAGGCCCT
GGCGAAGGCCGTGGCCTCGATGCTGTCATAGTAATACCAGTGTGCAGGTCCCAATGGCTGGTAT
TAATGTGACAGTCTCGGGGACGCGCAATTGGGTGGCCTCGATGCTGTCATAGTAATACCAGTGT
GCAGGTCCCAATGGCTGGTATTAATGTGACAGTCTCGGGGACGCGGGCGCGCCCGTGGCCTCGA
TGCTGTCATAGTAATACCAGTGTGCAGGTCCCAATGGCTGGTATTAATGTGACAGTCTCGGGGA
CGCGGGCCTGGACGCCGGCATCCGGGCTCAGGACCCCCCTCGAGCTCGCTGATCAGCCTCGACT
GTGCCTTCTAGTTGCCAGCCATCTGTTGTTTGCCCCTCCCCCGTGCCTTCCTTGACCCTGGAAG
GTGCCACTCCCACTGTCCTTTCCTAATAAAATGAGGAAATTGCATCGCATTGTCTGAGTAGGTG
TCATTCTATTCTGGGGGGTGGGGTGGGGCAGGACAGCAAGGGGGAGGATTGGGAAGACAATAGC
AGGCATGCTGGGGATGCGGTGGGCTCTATGGAAGCTTGAATTCAGCTGACGTGCCTCGGACCGC
TAGGAACCCCTAGTGATGGAGTTGGCCACTCCCTCTCTGCGCGCTCGCTCGCTCACTGAGGCCG
GGCGACCAAAGGTCGCCCGACGCCCGGGCTTTGCCCGGGCGGCCTCAGTGAGCGAGCGAGCGCG
CAGCTGCCTGGAGG
Exemplary pITR-CAG.mmu.1xmiR2i_3xmiR6u-26_KCNQ4codop
(SEQ ID NO: 253)
CTGCGCGCTCGCTCGCTCACTGAGGCCGCCCGGGCGTCGGGCGACCTTTGGTCGCCCGGCCTCA
GTGAGCGAGCGAGCGCGCAGAGAGGGAGTGGCCAACTCCATCACTAGGGGTTCCTGCGGCCGCA
CGCGTGACATTGATTATTGACTAGTTATTAATAGTAATCAATTACGGGGTCATTAGTTCATAGC
CCATATATGGAGTTCCGCGTTACATAACTTACGGTAAATGGCCCGCCTGGCTGACCGCCCAACG
ACCCCCGCCCATTGACGTCAATAATGACGTATGTTCCCATAGTAACGCCAATAGGGACTTTCCA
TTGACGTCAATGGGTGGACTATTTACGGTAAACTGCCCACTTGGCAGTACATCAAGTGTATCAT
ATGCCAAGTACGCCCCCTATTGACGTCAATGACGGTAAATGGCCCGCCTGGCATTATGCCCAGT
ACATGACCTTATGGGACTTTCCTACTTGGCAGTACATCTACGTATTAGTCATCGCTATTACCAT
GGGTCGAGGTGAGCCCCACGTTCTGCTTCACTCTCCCCATCTCCCCCCCCTCCCCACCCCCAAT
TTTGTATTTATTTATTTTTTAATTATTTTGTGCAGCGATGGGGGCGGGGGGGGGGGGGGCGCGC
GCCAGGCGGGGCGGGGCGGGGCGAGGGGCGGGGCGGGGCGAGGCGGAGAGGTGCGGCGGCAGCC
AATCAGAGCGGCGCGCTCCGAAAGTTTCCTTTTATGGCGAGGCGGCGGCGGCGGCGGCCCTATA
AAAAGCGAAGCGCGCGGCGGGCGGGAGTCGCTGCGTTGCCTTCGCCCCGTGCCCCGCTCCGCGC
CGCCTCGCGCCGCCCGCCCCGGCTCTGACTGACCGCGTTACTCCCACAGGTGAGCGGGCGGGAC
GGCCCTTCTCCTCCGGGCTGTAATTAGCGCTTGGTTTAATGACGGCTCGTTTCTTTTCTGTGGC
TGCGTGAAAGCCTTAAAGGGCTCCGGGAGGGCCCTTTGTGCGGGGGGGAGCGGCTCGGGGGGTG
CGTGCGTGTGTGTGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGCGGCCGGGGGCGG
TGCCCCGCGGTGCGGGGGGGCTGCGAGGGGAACAAAGGCTGCGTGCGGGGTGTGTGCGTGGGGG
GGTGAGCAGGGGGTGTGGGCGCGGCGGTCGGGCTGTAACCCCCCCCTGCACCCCCCTCCCCGAG
TTGCTGAGCACGGCCCGGCTTCGGGTGCGGGGCTCCGTGCGGGGCGTGGCGCGGGGCTCGCCGT
GCCGGGCGGGGGGTGGCGGCAGGTGGGGGTGCCGGGCGGGGCGGGGCCGCCTCGGGCCGGGGAG
GGCTCGGGGGAGGGGCGCGGCGGCCCCCGGAGCGCCGGCGGAGCAGGAGGACCAAGGCCCTGGC
GAAGGCCGTGGCCTCGTTGAGTCTTAGAGAGGCCCGGTGTGCAGGTCCCAATGGCCGGGTCTTA
CTGAGATTCCACGGGGACGCGGGCCTGGACGCCGGCATCCGGGCTCAGGACCCCCCTCTCTAGA
GCCTCTGCTAACCATGTTCATGCCTTCTTCTTTTTCCTACAGCTCCTGGGCAACGTGCTGGTTA
TTGTGACCGGTCGCTAGCCACCATGGCTGAAGCCCCTCCTAGAAGGCTTGGACTGGGACCTCCT
CCTGGGGATGCTCCTAGAGCTGAACTGGTGGCTCTGACAGCCGTGCAGTCTGAACAAGGCGAAG
CTGGTGGCGGCGGATCTCCACGTAGACTTGGACTGCTGGGAAGCCCTCTTCCTCCTGGTGCTCC
ACTTCCTGGACCTGGCAGTGGATCTGGATCTGCCTGTGGCCAGAGAAGCTCTGCCGCTCACAAG
AGATACCGGCGGCTGCAGAACTGGGTGTACAACGTGCTGGAAAGACCCAGAGGCTGGGCCTTCG
TGTACCACGTGTTCATCTTTCTGCTGGTGTTCAGCTGCCTGGTGCTGTCCGTGCTGAGCACCAT
CCAAGAACATCAAGAGCTGGCTAACGAGTGCCTGTTAATACTGGAGTTTGTGATGATTGTGGTG
TTCGGCCTCGAGTACATCGTCCGCGTTTGGAGCGCCGGCTGCTGCTGCAGATATAGAGGTTGGC
AAGGCAGATTCCGCTTCGCCAGAAAGCCCTTCTGCGTGATCGACTTCATCGTGTTCGTGGCCAG
CGTGGCCGTGATTGCTGCTGGCACACAGGGCAACATCTTCGCCACAAGCGCCCTGCGGAGCATG
CGGTTTCTGCAGATCCTGAGAATGGTCCGAATGGACAGAAGAGGCGGCACCTGGAAGCTGCTGG
GCTCTGTGGTGTACGCCCACAGCAAAGAGCTGATCACCGCCTGGTACATCGGATTTCTGGTGCT
GATCTTCGCCTCCTTCCTGGTGTACCTGGCCGAGAAGGACGCCAACAGCGACTTTAGCAGCTAC
GCCGACTCTCTTTGGTGGGGCACCATCACACTGACCACCATCGGCTACGGCGACAAGACCCCTC
ACACATGGCTGGGAAGAGTGCTGGCCGCTGGATTTGCTCTGCTGGGCATCAGCTTTTTCGCCCT
GCCTGCCGGAATCCTCGGATCTGGCTTTGCCCTGAAGGTGCAAGAGCAGCATAGACAAAAGCAT
TTTGAAAAGAGAAGAATGCCTGCCGCCAACCTGATTCAGGCCGCTTGGAGACTGTACAGCACCG
ACATGAGCAGAGCCTACCTGACCGCCACGTGGTATTATTACGATTCGATCCTGCCTAGCTTCCG
CGAACTGGCCCTGCTGTTTGAGCATGTGCAGAGAGCCAGAAACGGCGGCCTCAGACCTCTGGAA
GTTCGGAGAGCACCTGTGCCTGATGGCGCCCCTTCTAGATATCCTCCAGTGGCCACCTGTCACA
GACCCGGCAGCACATCTTTTTGCCCTGGCGAGTCTAGCCGGATGGGCATCAAGGACAGAATCAG
AATGGGCAGCAGCCAGCGGAGAACAGGCCCTTCTAAACAGCATCTGGCCCCTCCAACCATGCCT
ACAAGCCCTAGCTCTGAGCAAGTGGGCGAAGCCACCTCTCCTACCAAGGTGCAGAAGTCCTGGT
CCTTCAACGACCGGACCAGATTTAGAGCCAGCCTCCGGCTGAAGCCCAGAACCTCTGCCGAGGA
TGCCCCTTCTGAAGAGGTGGCCGAAGAGAAGTCCTACCAGTGCGAGCTGACCGTGGACGACATC
ATGCCAGCCGTGAAAACCGTGATAAGGTCTATACGCATCCTGAAGTTCCTGGTGGCCAAGCGGA
AGTTCAAAGAGACACTGCGGCCCTACGACGTGAAGGACGTGATCGAGCAGTATTCTGCCGGCCA
CCTGGACATGCTGGGCAGAATCAAGAGCCTGCAGACCAGAGTGGACCAGATCGTTGGAAGAGGC
CCAGGCGACAGAAAGGCCAGAGAGAAGGGCGATAAGGGCCCATCTGATGCCGAGGTTGTCGACG
AGATATCAATGATGGGCAGAGTCGTGAAAGTCGAAAAACAGGTGCAGAGCATCGAGCACAAGCT
GGACCTGCTGCTGGGATTCTACAGCCGGTGTCTGAGAAGCGGCACATCTGCATCTCTGGGCGCT
GTGCAGGTCCCACTGTTCGATCCTGATATTACGAGCGATTATCACAGCCCCGTGGACCACGAGG
ACATCTCCGTTTCTGCTCAGACCCTGAGCATCAGCAGATCCGTGTCCACCAACATGGACTAAcc
tgcaggcgccggcgGAGCAGGAGGACCAAGGCCCTGGCGAAGGCCGTGGCCTCGATGCTGTCAT
AGTAATACCAGTGTGCAGGTCCCAATGGCTGGTATTAATGTGACAGTCTCGGGGACGCGCAATT
GGGTGGCCTCGATGCTGTCATAGTAATACCAGTGTGCAGGTCCCAATGGCTGGTATTAATGTGA
CAGTCTCGGGGACGCGGGCGCGCCCGTGGCCTCGATGCTGTCATAGTAATACCAGTGTGCAGGT
CCCAATGGCTGGTATTAATGTGACAGTCTCGGGGACGCGGGCCTGGACGCCGGCATCCGGGCTC
AGGACCCCCCTCGAGCTCGCTGATCAGCCTCGACTGTGCCTTCTAGTTGCCAGCCATCTGTTGT
TTGCCCCTCCCCCGTGCCTTCCTTGACCCTGGAAGGTGCCACTCCCACTGTCCTTTCCTAATAA
AATGAGGAAATTGCATCGCATTGTCTGAGTAGGTGTCATTCTATTCTGGGGGGTGGGGTGGGGC
AGGACAGCAAGGGGGAGGATTGGGAAGACAATAGCAGGCATGCTGGGGATGCGGTGGGCTCTAT
GGAAGCTTGAATTCAGCTGACGTGCCTCGGACCGCTAGGAACCCCTAGTGATGGAGTTGGCCAC
TCCCTCTCTGCGCGCTCGCTCGCTCACTGAGGCCGGGCGACCAAAGGTCGCCCGACGCCCGGGC
TTTGCCCGGGCGGCCTCAGTGAGCGAGCGAGCGCGCAGCTGCCTGCAGG
Exemplary pITR-CAG.mmu.3xmiR2i_3xmiR6u-26_KCNQ4codop
(SEQ ID NO: 254)
CCTGCAGGCAGCTGCGCGCTCGCTCGCTCACTGAGGCCGCCCGGGCGTCGGGCGACCTTTGGTC
GCCCGGCCTCAGTGAGCGAGCGAGCGCGCAGAGAGGGAGTGGCCAACTCCATCACTAGGGGTTC
CTGCGGCCGCACGCGTGACATTGATTATTGACTAGTTATTAATAGTAATCAATTACGGGGTCAT
TAGTTCATAGCCCATATATGGAGTTCCGCGTTACATAACTTACGGTAAATGGCCCGCCTGGCTG
ACCGCCCAACGACCCCCGCCCATTGACGTCAATAATGACGTATGTTCCCATAGTAACGCCAATA
GGGACTTTCCATTGACGTCAATGGGTGGACTATTTACGGTAAACTGCCCACTTGGCAGTACATC
AAGTGTATCATATGCCAAGTACGCCCCCTATTGACGTCAATGACGGTAAATGGCCCGCCTGGCA
TTATGCCCAGTACATGACCTTATGGGACTTTCCTACTTGGCAGTACATCTACGTATTAGTCATC
GCTATTACCATGGGTCGAGGTGAGCCCCACGTTCTGCTTCACTCTCCCCATCTCCCCCCCCTCC
CCACCCCCAATTTTGTATTTATTTATTTTTTAATTATTTTGTGCAGCGATGGGGGCGGGGGGGG
GGGGGGCGCGCGCCAGGCGGGGCGGGGCGGGGCGAGGGGCGGGGCGGGGCGAGGCGGAGAGGTG
CGGCGGCAGCCAATCAGAGCGGCGCGCTCCGAAAGTTTCCTTTTATGGCGAGGCGGCGGCGGCG
GCGGCCCTATAAAAAGCGAAGCGCGCGGCGGGCGGGAGTCGCTGCGTTGCCTTCGCCCCGTGCC
CCGCTCCGCGCCGCCTCGCGCCGCCCGCCCCGGCTCTGACTGACCGCGTTACTCCCACAGGTGA
GCGGGCGGGACGGCCCTTCTCCTCCGGGCTGTAATTAGCGCTTGGTTTAATGACGGCTCGTTTC
TTTTCTGTGGCTGCGTGAAAGCCTTAAAGGGCTCCGGGAGGGCCCTTTGTGCGGGGGGGAGCGG
CTCGGGGGGTGCGTGCGTGTGTGTGTGCGTGGGGAGCGCCGCGTGCGGCCCGCGCTGCCCGGCG
GCTGTGAGCGCTGCGGGCGCGGCGCGGGGCTTTGTGCGCTCCGCGTGTGCGCGAGGGGAGCGCG
GCCGGGGGCGGTGCCCCGCGGTGCGGGGGGGCTGCGAGGGGAACAAAGGCTGCGTGCGGGGTGT
GTGCGTGGGGGGGTGAGCAGGGGGTGTGGGCGCGGCGGTCGGGCTGTAACCCCCCCCTGCACCC
CCCTCCCCGAGTTGCTGAGCACGGCCCGGCTTCGGGTGCGGGGCTCCGTGCGGGGCGTGGCGCG
GGGCTCGCCGTGCCGGGCGGGGGGTGGCGGCAGGTGGGGGTGCCGGGCGGGGCGGGGCCGCCTC
GGGCCGGGGAGGGCTCGGGGGAGGGGCGCGGCGGCCCCCGGAGCGCCGGCGGAGCAGGAGGACC
AAGGCCCTGGCGAAGGCCGTGGCCTCGTTGAGTCTTAGAGAGGCCCGGTGTGCAGGTCCCAATG
GCCGGGTCTTACTGAGATTCCACGGGGACGCGGGATCCGGTGGCCTCGTTGAGTCTTAGAGAGG
CCCGGTGTGCAGGTCCCAATGGCCGGGTCTTACTGAGATTCCACGGGGACGCGGGTACCCGTGG
CCTCGTTGAGTCTTAGAGAGGCCCGGTGTGCAGGTCCCAATGGCCGGGTCTTACTGAGATTCCA
CGGGGACGCGGGCCTGGACGCCGGCATCCGGGCTCAGGACCCCCCTCTCTAGAGCCTCTGCTAA
CCATGTTCATGCCTTCTTCTTTTTCCTACAGCTCCTGGGCAACGTGCTGGTTATTGTGACCGGT
CGCTAGCCACCATGGCTGAAGCCCCTCCTAGAAGGCTTGGACTGGGACCTCCTCCTGGGGATGC
TCCTAGAGCTGAACTGGTGGCTCTGACAGCCGTGCAGTCTGAACAAGGCGAAGCTGGTGGCGGC
GGATCTCCACGTAGACTTGGACTGCTGGGAAGCCCTCTTCCTCCTGGTGCTCCACTTCCTGGAC
CTGGCAGTGGATCTGGATCTGCCTGTGGCCAGAGAAGCTCTGCCGCTCACAAGAGATACCGGCG
GCTGCAGAACTGGGTGTACAACGTGCTGGAAAGACCCAGAGGCTGGGCCTTCGTGTACCACGTG
TTCATCTTTCTGCTGGTGTTCAGCTGCCTGGTGCTGTCCGTGCTGAGCACCATCCAAGAACATC
AAGAGCTGGCTAACGAGTGCCTGTTAATACTGGAGTTTGTGATGATTGTGGTGTTCGGCCTCGA
GTACATCGTCCGCGTTTGGAGCGCCGGCTGCTGCTGCAGATATAGAGGTTGGCAAGGCAGATTC
CGCTTCGCCAGAAAGCCCTTCTGCGTGATCGACTTCATCGTGTTCGTGGCCAGCGTGGCCGTGA
TTGCTGCTGGCACACAGGGCAACATCTTCGCCACAAGCGCCCTGCGGAGCATGCGGTTTCTGCA
GATCCTGAGAATGGTCCGAATGGACAGAAGAGGCGGCACCTGGAAGCTGCTGGGCTCTGTGGTG
TACGCCCACAGCAAAGAGCTGATCACCGCCTGGTACATCGGATTTCTGGTGCTGATCTTCGCCT
CCTTCCTGGTGTACCTGGCCGAGAAGGACGCCAACAGCGACTTTAGCAGCTACGCCGACTCTCT
TTGGTGGGGCACCATCACACTGACCACCATCGGCTACGGCGACAAGACCCCTCACACATGGCTG
GGAAGAGTGCTGGCCGCTGGATTTGCTCTGCTGGGCATCAGCTTTTTCGCCCTGCCTGCCGGAA
TCCTCGGATCTGGCTTTGCCCTGAAGGTGCAAGAGCAGCATAGACAAAAGCATTTTGAAAAGAG
AAGAATGCCTGCCGCCAACCTGATTCAGGCCGCTTGGAGACTGTACAGCACCGACATGAGCAGA
GCCTACCTGACCGCCACGTGGTATTATTACGATTCGATCCTGCCTAGCTTCCGCGAACTGGCCC
TGCTGTTTGAGCATGTGCAGAGAGCCAGAAACGGCGGCCTCAGACCTCTGGAAGTTCGGAGAGC
ACCTGTGCCTGATGGCGCCCCTTCTAGATATCCTCCAGTGGCCACCTGTCACAGACCCGGCAGC
ACATCTTTTTGCCCTGGCGAGTCTAGCCGGATGGGCATCAAGGACAGAATCAGAATGGGCAGCA
GCCAGCGGAGAACAGGCCCTTCTAAACAGCATCTGGCCCCTCCAACCATGCCTACAAGCCCTAG
CTCTGAGCAAGTGGGCGAAGCCACCTCTCCTACCAAGGTGCAGAAGTCCTGGTCCTTCAACGAC
CGGACCAGATTTAGAGCCAGCCTCCGGCTGAAGCCCAGAACCTCTGCCGAGGATGCCCCTTCTG
AAGAGGTGGCCGAAGAGAAGTCCTACCAGTGCGAGCTGACCGTGGACGACATCATGCCAGCCGT
GAAAACCGTGATAAGGTCTATACGCATCCTGAAGTTCCTGGTGGCCAAGCGGAAGTTCAAAGAG
ACACTGCGGCCCTACGACGTGAAGGACGTGATCGAGCAGTATTCTGCCGGCCACCTGGACATGC
TGGGCAGAATCAAGAGCCTGCAGACCAGAGTGGACCAGATCGTTGGAAGAGGCCCAGGCGACAG
AAAGGCCAGAGAGAAGGGCGATAAGGGCCCATCTGATGCCGAGGTTGTCGACGAGATATCAATG
ATGGGCAGAGTCGTGAAAGTCGAAAAACAGGTGCAGAGCATCGAGCACAAGCTGGACCTGCTGC
TGGGATTCTACAGCCGGTGTCTGAGAAGCGGCACATCTGCATCTCTGGGCGCTGTGCAGGTCCC
ACTGTTCGATCCTGATATTACGAGCGATTATCACAGCCCCGTGGACCACGAGGACATCTCCGTT
TCTGCTCAGACCCTGAGCATCAGCAGATCCGTGTCCACCAACATGGACTAAcctgcaggcgccg
gcgGAGCAGGAGGACCAAGGCCCTGGCGAAGGCCGTGGCCTCGATGCTGTCATAGTAATACCAG
TGTGCAGGTCCCAATGGCTGGTATTAATGTGACAGTCTCGGGGACGCGCAATTGGGTGGCCTCG
ATGCTGTCATAGTAATACCAGTGTGCAGGTCCCAATGGCTGGTATTAATGTGACAGTCTCGGGG
ACGCGGGCGCGCCCGTGGCCTCGATGCTGTCATAGTAATACCAGTGTGCAGGTCCCAATGGCTG
GTATTAATGTGACAGTCTCGGGGACGCGGGCCTGGACGCCGGCATCCGGGCTCAGGACCCCCCT
CGAGCTCGCTGATCAGCCTCGACTGTGCCTTCTAGTTGCCAGCCATCTGTTGTTTGCCCCTCCC
CCGTGCCTTCCTTGACCCTGGAAGGTGCCACTCCCACTGTCCTTTCCTAATAAAATGAGGAAAT
TGCATCGCATTGTCTGAGTAGGTGTCATTCTATTCTGGGGGGTGGGGTGGGGCAGGACAGCAAG
GGGGAGGATTGGGAAGACAATAGCAGGCATGCTGGGGATGCGGTGGGCTCTATGGAAGCTTGAA
TTCAGCTGACGTGCCTCGGACCGCTAGGAACCCCTAGTGATGGAGTTGGCCACTCCCTCTCTGC
GCGCTCGCTCGCTCACTGAGGCCGGGCGACCAAAGGTCGCCCGACGCCCGGGCTTTGCCCGGGC
GGCCTCAGTGAGCGAGCGAGCGCGCAGCTGCCTGCAGG

Exemplary hKCNQ4 codon optimized to resist microRNA Construct Sequences

Exemplary pITR-CMV.hKCNQ4codop_v2.mScarlet sequence
(SEQ ID NO: 255)
CCTGCAGGCAGCTGCGCGCTCGCTCGCTCACTGAGGCCGCCCGGGCGTCGGGCGACCTTTGGTC
GCCCGGCCTCAGTGAGCGAGCGAGCGCGCAGAGAGGGAGTGGCCAACTCCATCACTAGGGGTTC
CTGCGGCCGCACGCGTCTAGATCCCATATATGGAGTTCCGCGTTACATAACTTACGGTAAATGG
CCCGCCTGGCTGACCGCCCAACGACCCCCGCCCATTGACGTCAATAATGACGTATGTTCCCATA
GTAACGCCAATAGGGACTTTCCATTGACGTCAATGGGTGGAGTATTTACGGTAAACTGCCCACT
TGGCAGTACATCAAGTGTATCATATGCCAAGTACGCCCCCTATTGACGTCAATGACGGTAAATG
GCCCGCCTGGCATTATGCCCAGTACATGACCTTATGGGACTTTCCTACTTGGCAGTACATCTAC
GTATTAGTCATCGCTATTACCATGGTGATGCGGTTTTGGCAGTACATCAATGGGCGTGGATAGC
GGTTTGACTCACGGGGATTTCCAAGTCTCCACCCCATTGACGTCAATGGGAGTTTGTTTTGGCA
CCAAAATCAACGGGACTTTCCAAAATGTCGTAACAACTCCGCCCCATTGACGCAAATGGGCGGT
AGGCGTGTACGGTGGGAGGTCTATATAAGCAGAGCTCGTTTAGTGAACCGTcagatcgcctgga
gacgcACCGGTGCCACCATGGCTGAAGCCCCTCCTAGAAGGCTTGGACTGGGACCTCCTCCTGG
GGATGCTCCTAGAGCTGAACTGGTGGCTCTGACAGCCGTGCAGTCTGAACAAGGCGAAGCTGGT
GGCGGCGGATCTCCACGTAGACTTGGACTGCTGGGAAGCCCTCTTCCTCCTGGTGCTCCACTTC
CTGGACCTGGCAGTGGATCTGGATCTGCCTGTGGCCAGAGAAGCTCTGCCGCTCACAAGAGATA
CCGGCGGCTGCAGAACTGGGTGTACAACGTGCTGGAAAGACCCAGAGGCTGGGCCTTCGTGTAC
CACGTGTTCATCTTTCTGCTGGTGTTCAGCTGCCTGGTGCTGTCCGTGCTGAGCACCATCCAAG
AACATCAAGAGCTGGCTAACGAGTGCCTGTTAATACTGGAGTTTGTGATGATTGTGGTGTTCGG
CCTCGAGTACATCGTCCGCGTTTGGAGCGCCGGCTGCTGCTGCAGATATAGAGGTTGGCAAGGC
AGATTCCGCTTCGCCAGAAAGCCCTTCTGCGTGATCGACTTCATCGTGTTCGTGGCCAGCGTGG
CCGTGATTGCTGCTGGCACACAGGGCAACATCTTCGCCACAAGCGCCCTGCGGAGCATGCGGTT
TCTGCAGATCCTGAGAATGGTCCGAATGGACAGAAGAGGCGGCACCTGGAAGCTGCTGGGCTCT
GTGGTGTACGCCCACAGCAAAGAGCTGATCACCGCCTGGTACATCGGATTTCTGGTGCTGATCT
TCGCCTCCTTCCTGGTGTACCTGGCCGAGAAGGACGCCAACAGCGACTTTAGCAGCTACGCCGA
CTCTCTTTGGTGGGGCACCATCACACTGACCACCATCGGCTACGGCGACAAGACCCCTCACACA
TGGCTGGGAAGAGTGCTGGCCGCTGGATTTGCTCTGCTGGGCATCAGCTTTTTCGCCCTGCCTG
CCGGAATCCTCGGATCTGGCTTTGCCCTGAAGGTGCAAGAGCAGCATAGACAAAAGCATTTTGA
AAAGAGAAGAATGCCTGCCGCCAACCTGATTCAGGCCGCTTGGAGACTGTACAGCACCGACATG
AGCAGAGCCTACCTGACCGCCACGTGGTATTATTACGATTCGATCCTGCCTAGCTTCCGCGAAC
TGGCCCTGCTGTTTGAGCATGTGCAGAGAGCCAGAAACGGCGGCCTCAGACCTCTGGAAGTTCG
GAGAGCACCTGTGCCTGATGGCGCCCCTTCTAGATATCCTCCAGTGGCCACCTGTCACAGACCC
GGCAGCACATCTTTTTGCCCTGGCGAGTCTAGCCGGATGGGCATCAAGGACAGAATCAGAATGG
GCAGCAGCCAGCGGAGAACAGGCCCTTCTAAACAGCATCTGGCCCCTCCAACCATGCCTACAAG
CCCTAGCTCTGAGCAAGTGGGCGAAGCCACCTCTCCTACCAAGGTGCAGAAGTCCTGGTCCTTC
AACGACCGGACCAGATTTAGAGCCAGCCTCCGGCTGAAGCCCAGAACCTCTGCCGAGGATGCCC
CTTCTGAAGAGGTGGCCGAAGAGAAGTCCTACCAGTGCGAGCTGACCGTGGACGACATCATGCC
AGCCGTGAAAACCGTGATAAGGTCTATACGCATCCTGAAGTTCCTGGTGGCCAAGCGGAAGTTC
AAAGAGACACTGCGGCCCTACGACGTGAAGGACGTGATCGAGCAGTATTCTGCCGGCCACCTGG
ACATGCTGGGCAGAATCAAGAGCCTGCAGACCAGAGTGGACCAGATCGTTGGAAGAGGCCCAGG
CGACAGAAAGGCCAGAGAGAAGGGCGATAAGGGCCCATCTGATGCCGAGGTTGTCGACGAGATA
TCAATGATGGGCAGAGTCGTGAAAGTCGAAAAACAGGTGCAGAGCATCGAGCACAAGCTGGACC
TGCTGCTGGGATTCTACAGCCGGTGTCTGAGAAGCGGCACATCTGCATCTCTGGGCGCTGTGCA
GGTCCCACTGTTCGATCCTGATATTACGAGCGATTATCACAGCCCCGTGGACCACGAGGACATC
TCCGTTTCTGCTCAGACCCTGAGCATCAGCAGATCCGTGTCCACCAACATGGACGGATCCCGGG
CTGACTACAAAGACCATGACGGTGATTATAAAGATCATGACATCGACTACAAGGATGACGATGA
CAAGGGCTCCGGAGAGGGCAGAGGAAGTCTGCTAACATGCGGTGACGTCGAGGAGAATCCTGGC
CCAATGGTGAGCAAGGGCGAGGCAGTGATCAAGGAGTTCATGCGGTTCAAGGTGCACATGGAGG
GCTCCATGAACGGCCACGAGTTCGAGATCGAGGGCGAGGGCGAGGGCCGCCCCTACGAGGGCAC
CCAGACCGCCAAGCTGAAGGTGACCAAGGGTGGCCCCCTGCCCTTCTCCTGGGACATCCTGTCC
CCTCAGTTCATGTACGGCTCCAGGGCCTTCATCAAGCACCCCGCCGACATCCCCGACTACTATA
AGCAGTCCTTCCCCGAGGGCTTCAAGTGGGAGCGCGTGATGAACTTCGAGGACGGCGGCGCCGT
GACCGTGACCCAGGACACCTCCCTGGAGGACGGCACCCTGATCTACAAGGTGAAGCTCCGCGGC
ACCAACTTCCCTCCTGACGGCCCCGTAATGCAGAAGAAGACAATGGGCTGGGAAGCGTCCACCG
AGCGGTTGTACCCCGAGGACGGCGTGCTGAAGGGCGACATTAAGATGGCCCTGCGCCTGAAGGA
CGGCGGCCGCTACCTGGCGGACTTCAAGACCACCTACAAGGCCAAGAAGCCCGTGCAGATGCCC
GGCGCCTACAACGTCGACCGCAAGTTGGACATCACCTCCCACAACGAGGACTACACCGTGGTGG
AACAGTACGAACGCTCCGAGGGCCGCCACTCCACCGGCGGCATGGACGAGCTGTACAAGTAATA
AGAGCTCGCTGATCAGCCTCGACTGTGCCTTCTAGTTGCCAGCCATCTGTTGTTTGCCCCTCCC
CCGTGCCTTCCTTGACCCTGGAAGGTGCCACTCCCACTGTCCTTTCCTAATAAAATGAGGAAAT
TGCATCGCATTGTCTGAGTAGGTGTCATTCTATTCTGGGGGGTGGGGTGGGGCAGGACAGCAAG
GGGGAGGATTGGGAAGACAATAGCAGGCATGCTGGGGATGCGGTGGGCTCTATGGAAGCTTGAA
TTCAGCTGACGTGCCTCGGACCGCTAGGAACCCCTAGTGATGGAGTTGGCCACTCCCTCTCTGC
GCGCTCGCTCGCTCACTGAGGCCGGGCGACCAAAGGTCGCCCGACGCCCGGGCTTTGCCCGGGC
GGCCTCAGTGAGCGAGCGAGCGCGCAGCTGCCTGCAGGGGCGCCTGATGCGGTATTTTCTCCTT
ACGCATCTGTGCGGTATTTCACACCGCATACGTCAAAGCAACCATAGTACGCGCCCTGTAGCGG
CGCATTAAGCGCGGCGGGTGTGGTGGTTACGCGCAGCGTGACCGCTACACTTGCCAGCGCCCTA
GCGCCCGCTCCTTTCGCTTTCTTCCCTTCCTTTCTCGCCACGTTCGCCGGCTTTCCCCGTCAAG
CTCTAAATCGGGGGCTCCCTTTAGGGTTCCGATTTAGTGCTTTACGGCACCTCGACCCCAAAAA
ACTTGATTTGGGTGATGGTTCACGTAGTGGGCCATCGCCCTGATAGACGGTTTTTCGCCCTTTG
ACGTTGGAGTCCACGTTCTTTAATAGTGGACTCTTGTTCCAAACTGGAACAACACTCAACCCTA
TCTCGGGCTATTCTTTTGATTTATAAGGGATTTTGCCGATTTCGGCCTATTGGTTAAAAAATGA
GCTGATTTAACAAAAATTTAACGCGAATTTTAACAAAATATTAACGTTTACAATTTTATGGTGC
ACTCTCAGTACAATCTGCTCTGATGCCGCATAGTTAAGCCAGCCCCGACACCCGCCAACACCCG
CTGACGCGCCCTGACGGGCTTGTCTGCTCCCGGCATCCGCTTACAGACAAGCTGTGACCGTCTC
CGGGAGCTGCATGTGTCAGAGGTTTTCACCGTCATCACCGAAACGCGCGAGACGAAAGGGCCTC
GTGATACGCCTATTTTTATAGGTTAATGTCATGAACAATAAAACTGTCTGCTTACATAAACAGT
AATACAAGGGGTGTTATGAGCCATATTCAACGGGAAACGTCGAGGCCGCGATTAAATTCCAACA
TGGATGCTGATTTATATGGGTATAAATGGGCTCGCGATAATGTCGGGCAATCAGGTGCGACAAT
CTATCGCTTGTATGGGAAGCCCGATGCGCCAGAGTTGTTTCTGAAACATGGCAAAGGTAGCGTT
GCCAATGATGTTACAGATGAGATGGTCAGACTAAACTGGCTGACGGAATTTATGCCTCTTCCGA
CCATCAAGCATTTTATCCGTACTCCTGATGATGCATGGTTACTCACCACTGCGATCCCCGGAAA
AACAGCATTCCAGGTATTAGAAGAATATCCTGATTCAGGTGAAAATATTGTTGATGCGCTGGCA
GTGTTCCTGCGCCGGTTGCATTCGATTCCTGTTTGTAATTGTCCTTTTAACAGCGATCGCGTAT
TTCGTCTCGCTCAGGCGCAATCACGAATGAATAACGGTTTGGTTGATGCGAGTGATTTTGATGA
CGAGCGTAATGGCTGGCCTGTTGAACAAGTCTGGAAAGAAATGCATAAACTTTTGCCATTCTCA
CCGGATTCAGTCGTCACTCATGGTGATTTCTCACTTGATAACCTTATTTTTGACGAGGGGAAAT
TAATAGGTTGTATTGATGTTGGACGAGTCGGAATCGCAGACCGATACCAGGATCTTGCCATCCT
ATGGAACTGCCTCGGTGAGTTTTCTCCTTCATTACAGAAACGGCTTTTTCAAAAATATGGTATT
GATAATCCTGATATGAATAAATTGCAGTTTCATTTGATGCTCGATGAGTTTTTCTAATCTCATG
ACCAAAATCCCTTAACGTGAGTTTTCGTTCCACTGAGCGTCAGACCCCGTAGAAAAGATCAAAG
GATCTTCTTGAGATCCTTTTTTTCTGCGCGTAATCTGCTGCTTGCAAACAAAAAAACCACCGCT
ACCAGCGGTGGTTTGTTTGCCGGATCAAGAGCTACCAACTCTTTTTCCGAAGGTAACTGGCTTC
AGCAGAGCGCAGATACCAAATACTGTCCTTCTAGTGTAGCCGTAGTTAGGCCACCACTTCAAGA
ACTCTGTAGCACCGCCTACATACCTCGCTCTGCTAATCCTGTTACCAGTGGCTGCTGCCAGTGG
CGATAAGTCGTGTCTTACCGGGTTGGACTCAAGACGATAGTTACCGGATAAGGCGCAGCGGTCG
GGCTGAACGGGGGGTTCGTGCACACAGCCCAGCTTGGAGCGAACGACCTACACCGAACTGAGAT
ACCTACAGCGTGAGCTATGAGAAAGCGCCACGCTTCCCGAAGGGAGAAAGGCGGACAGGTATCC
GGTAAGCGGCAGGGTCGGAACAGGAGAGCGCACGAGGGAGCTTCCAGGGGGAAACGCCTGGTAT
CTTTATAGTCCTGTCGGGTTTCGCCACCTCTGACTTGAGCGTCGATTTTTGTGATGCTCGTCAG
GGGGGCGGAGCCTATGGAAAAACGCCAGCAACGCGGCCTTTTTACGGTTCCTGGCCTTTTGCTG
GCCTTTTGCTCACATGT

Exemplary wildtype KCNQ4 Construct Sequences

Exemplary AAV2 ITR-CMV enhancer-CMV promoter-human KCNQ4-3x FLAG-T2A-
mScarlet-bGH poly(A) signal-AAV2 ITR sequence
(SEQ ID NO: 256)
CCTGCAGGCAGCTGCGCGCTCGCTCGCTCACTGAGGCCGCCCGGGCGTCGGGCGACCTTTGGTC
GCCCGGCCTCAGTGAGCGAGCGAGCGCGCAGAGAGGGAGTGGCCAACTCCATCACTAGGGGTTC
CTGCGGCCGCACGCGTCTAGATCCCATATATGGAGTTCCGCGTTACATAACTTACGGTAAATGG
CCCGCCTGGCTGACCGCCCAACGACCCCCGCCCATTGACGTCAATAATGACGTATGTTCCCATA
GTAACGCCAATAGGGACTTTCCATTGACGTCAATGGGTGGAGTATTTACGGTAAACTGCCCACT
TGGCAGTACATCAAGTGTATCATATGCCAAGTACGCCCCCTATTGACGTCAATGACGGTAAATG
GCCCGCCTGGCATTATGCCCAGTACATGACCTTATGGGACTTTCCTACTTGGCAGTACATCTAC
GTATTAGTCATCGCTATTACCATGGTGATGCGGTTTTGGCAGTACATCAATGGGCGTGGATAGC
GGTTTGACTCACGGGGATTTCCAAGTCTCCACCCCATTGACGTCAATGGGAGTTTGTTTTGGCA
CCAAAATCAACGGGACTTTCCAAAATGTCGTAACAACTCCGCCCCATTGACGCAAATGGGCGGT
AGGCGTGTACGGTGGGAGGTCTATATAAGCAGAGCTCGTTTAGTGAACCGTcagatcgcctgga
gacgcACCGGTGCCACCATGGCCGAGGCCCCCCCGCGCCGCCTCGGCCTGGGTCCCCCGCCCGG
GGACGCCCCCCGCGCGGAGCTAGTGGCGCTCACGGCCGTGCAGAGCGAACAGGGCGAGGCGGGC
GGGGGCGGCTCCCCGCGCCGCCTCGGCCTCCTGGGCAGCCCCCTGCCGCCGGGCGCGCCCCTCC
CTGGGCCGGGCTCCGGCTCGGGCTCCGCCTGCGGCCAGCGCTCCTCGGCCGCGCACAAGCGCTA
CCGCCGCCTGCAGAACTGGGTCTACAACGTGCTGGAGCGGCCCCGCGGCTGGGCCTTCGTCTAC
CACGTCTTCATATTTTTGCTGGTCTTCAGCTGCCTGGTGCTGTCTGTGCTGTCCACTATCCAGG
AGCACCAGGAACTTGCCAACGAGTGTCTCCTCATCTTGGAATTCGTGATGATCGTGGTTTTCGG
CTTGGAGTACATCGTCCGGGTCTGGTCCGCCGGATGCTGCTGCCGCTACCGAGGATGGCAGGGT
CGCTTCCGCTTTGCCAGAAAGCCCTTCTGTGTCATCGACTTCATCGTGTTCGTGGCCTCGGTGG
CCGTCATCGCCGCGGGTACCCAGGGCAACATCTTCGCCACGTCCGCGCTGCGCAGCATGCGCTT
CCTGCAGATCCTGCGCATGGTGCGCATGGACCGCCGCGGCGGCACCTGGAAGCTGCTGGGCTCA
GTGGTCTACGCGCATAGCAAGGAGCTGATCACCGCCTGGTACATCGGGTTCCTGGTGCTCATCT
TCGCCTCCTTCCTGGTCTACCTGGCTGAGAAGGACGCCAACTCCGACTTCTCCTCCTACGCCGA
CTCGCTCTGGTGGGGGACGATTACATTGACAACCATCGGCTATGGTGACAAGACACCGCACACA
TGGCTGGGCAGGGTCCTGGCTGCTGGCTTCGCCTTACTGGGCATCTCTTTCTTTGCCCTGCCTG
CCGGCATCCTAGGCTCCGGCTTTGCCCTGAAGGTCCAGGAGCAGCACCGGCAGAAGCACTTCGA
GAAGCGGAGGATGCCGGCAGCCAACCTCATCCAGGCTGCCTGGCGCCTGTACTCCACCGATATG
AGCCGGGCCTACCTGACAGCCACCTGGTACTACTATGACAGTATCCTCCCATCCTTCAGAGAGC
TGGCCCTCTTGTTTGAGCACGTGCAACGGGCCCGCAATGGGGGCCTACGGCCCCTGGAGGTGCG
GCGGGCGCCGGTACCCGACGGAGCACCCTCCCGTTACCCGCCCGTTGCCACCTGCCACCGGCCG
GGCAGCACCTCCTTCTGCCCTGGGGAAAGCAGCCGGATGGGCATCAAAGACCGCATCCGCATGG
GCAGCTCCCAGCGGCGGACGGGTCCTTCCAAGCAGCATCTGGCACCTCCAACAATGCCCACCTC
CCCAAGCAGCGAGCAGGTGGGTGAGGCCACCAGCCCCACCAAGGTGCAAAAGAGCTGGAGCTTC
AATGACCGCACCCGCTTCCGGGCATCTCTGAGACTCAAACCCCGCACCTCTGCTGAGGATGCCC
CCTCAGAGGAAGTAGCAGAGGAGAAGAGCTACCAGTGTGAGCTCACGGTGGACGACATCATGCC
TGCTGTGAAGACAGTCATCCGCTCCATCAGGATTCTCAAGTTCCTGGTGGCCAAAAGGAAATTC
AAGGAGACACTGCGACCGTACGACGTGAAGGACGTCATTGAGCAGTACTCAGCAGGCCACCTGG
ACATGCTGGGCCGGATCAAGAGCCTGCAAACTCGGGTGGACCAAATTGTGGGTCGGGGGCCCGG
GGACAGGAAGGCCCGGGAGAAGGGCGACAAGGGGCCCTCCGACGCGGAGGTGGTGGATGAAATC
AGCATGATGGGACGCGTGGTCAAGGTGGAGAAGCAGGTGCAGTCCATCGAGCACAAGCTGGACC
TGCTGTTGGGCTTCTATTCGCGCTGCCTGCGCTCTGGCACCTCGGCCAGCCTGGGCGCCGTGCA
AGTGCCGCTGTTCGACCCCGACATCACCTCCGACTACCACAGCCCTGTGGACCACGAGGACATC
TCCGTCTCCGCACAGACGCTCAGCATCTCCCGCTCGGTCAGCACCAACATGGACGGATCCCGGG
CTGAGTACAAAGACCATGACGGTGATTATAAAGATCATGACATCGACTACAAGGATGACGATGA
CAAGGGCTCCGGAGAGGGCAGAGGAAGTCTGCTAACATGCGGTGACGTCGAGGAGAATCCTGGC
CCAATGGTGAGCAAGGGCGAGGCAGTGATCAAGGAGTTCATGCGGTTCAAGGTGCACATGGAGG
GCTCCATGAACGGCCACGAGTTCGAGATCGAGGGCGAGGGCGAGGGCCGCCCCTACGAGGGCAC
CCAGACCGCCAAGCTGAAGGTGACCAAGGGTGGCCCCCTGCCCTTCTCCTGGGACATCCTGTCC
CCTCAGTTCATGTACGGCTCCAGGGCCTTCATCAAGCACCCCGCCGACATCCCCGACTACTATA
AGCAGTCCTTCCCCGAGGGCTTCAAGTGGGAGCGCGTGATGAACTTCGAGGACGGCGGCGCCGT
GACCGTGACCCAGGACACCTCCCTGGAGGACGGCACCCTGATCTACAAGGTGAAGCTCCGCGGC
ACCAACTTCCCTCCTGACGGCCCCGTAATGCAGAAGAAGACAATGGGCTGGGAAGCGTCCACCG
AGCGGTTGTACCCCGAGGACGGCGTGCTGAAGGGCGACATTAAGATGGCCCTGCGCCTGAAGGA
CGGCGGCCGCTACCTGGCGGACTTCAAGACCACCTACAAGGCCAAGAAGCCCGTGCAGATGCCC
GGCGCCTACAACGTCGACCGCAAGTTGGACATCACCTCCCACAACGAGGACTACACCGTGGTGG
AACAGTACGAACGCTCCGAGGGCCGCCACTCCACCGGCGGCATGGACGAGCTGTACAAGTAATA
AGAGCTCGCTGATCAGCCTCGACTGTGCCTTCTAGTTGCCAGCCATCTGTTGTTTGCCCCTCCC
CCGTGCCTTCCTTGACCCTGGAAGGTGCCACTCCCACTGTCCTTTCCTAATAAAATGAGGAAAT
TGCATCGCATTGTCTGAGTAGGTGTCATTCTATTCTGGGGGGTGGGGTGGGGCAGGACAGCAAG
GGGGAGGATTGGGAAGACAATAGCAGGCATGCTGGGGATGCGGTGGGCTCTATGGAAGCTTGAA
TTCAGCTGACGTGCCTCGGACCGCTAGGAACCCCTAGTGATGGAGTTGGCCACTCCCTCTCTGC
GCGCTCGCTCGCTCACTGAGGCCGGGCGACCAAAGGTCGCCCGACGCCCGGGCTTTGCCCGGGC
GGCCTCAGTGAGCGAGCGAGCGCGCAGCTGCCTGCAGG
Exemplary AAV2 ITR-CMV enhancer-CMV promoter-mouse KCNQ4-3x FLAG-T2A-
mScarlet-bGH poly(A) signal-AAV2 ITR sequence
(SEQ ID NO: 257)
CCTGCAGGCAGCTGCGCGCTCGCTCGCTCACTGAGGCCGCCCGGGCGTCGGGCGACCTTTGGTC
GCCCGGCCTCAGTGAGCGAGCGAGCGCGCAGAGAGGGAGTGGCCAACTCCATCACTAGGGGTTC
CTGCGGCCGCACGCGTCTAGATCCCATATATGGAGTTCCGCGTTACATAACTTACGGTAAATGG
CCCGCCTGGCTGACCGCCCAACGACCCCCGCCCATTGACGTCAATAATGACGTATGTTCCCATA
GTAACGCCAATAGGGACTTTCCATTGACGTCAATGGGTGGAGTATTTACGGTAAACTGCCCACT
TGGCAGTACATCAAGTGTATCATATGCCAAGTACGCCCCCTATTGACGTCAATGACGGTAAATG
GCCCGCCTGGCATTATGCCCAGTACATGACCTTATGGGACTTTCCTACTTGGCAGTACATCTAC
GTATTAGTCATCGCTATTACCATGGTGATGCGGTTTTGGCAGTACATCAATGGGCGTGGATAGC
GGTTTGACTCACGGGGATTTCCAAGTCTCCACCCCATTGACGTCAATGGGAGTTTGTTTTGGCA
CCAAAATCAACGGGACTTTCCAAAATGTCGTAACAACTCCGCCCCATTGACGCAAATGGGCGGT
AGGCGTGTACGGTGGGAGGTCTATATAAGCAGAGCTCGTTTAGTGAACCGTcagatcgcctgga
gacgcACCGGTGCCACCATGGCCGAGGCCCCCCCGCGCCGCCTCGGCCTGGGCCCCCCGCCCGG
GGACGCCCCCCGCGCGGAGTTGGTGGCGCTCACGGCCGTGCAGAGTGAACAGGGCGAGGCGGGC
GGGGGCGGCTCTCCGCGTCGCCTCGGCCTTCTGGGCAGCCCCCTGCCGCCGGGCGCGCCCCTCC
CTGGGCCGGGCTCCGGCTCGGGCTCCGCCTGCGGCGGCCAGCGCTCCTCCGCCGCGCAGAAGCG
CTACCGCCGCCTGCAGAACTGGGTCTACAACGTGCTGGAGCGGCCCCGCGGGTGGGCCTTCGTC
TACCACGTCTTCATATTTTTGCTAGTCTTCAGCTGCCTGGTGCTGTCTGTACTGTCCACCATCC
AGGAGCACCAGGAACTTGCCAACGAGTGTCTCCTTATCTTGGAATTCGTGATGATTGTGGTCTT
TGGCTTGGAGTATATAATCCGTGTCTGGTCGGCCGGATGCTGTTGTCGCTACAGAGGATGGCAG
GGACGCTTTCGCTTCGCCAGGAAACCCTTCTGTGTCATCGACTTCATCGTGTTCGTGGCCTCGG
TGGCAGTGATAGCTGCGGGCACACAAGGCAACATCTTTGCTACGTCCGCGTTGCGCAGTATGCG
CTTCCTACAGATCCTGCGCATGGTGCGTATGGATCGCCGCGGTGGCACCTGGAAGCTGTTGGGA
TCCGTGGTCTATGCGCACAGTAAGGAGCTGATCACCGCCTGGTACATCGGGTTCCTGGTGCTCA
TCTTTGCCTCTTTCCTGGTCTACCTGGCTGAGAAGGATGCCAACTCTGACTTCTCCTCCTATGC
CGACTCGCTCTGGTGGGGGACGATCACACTGACGACCATTGGCTATGGTGACAAGACGCCACAT
ACATGGCTGGGCAGGGTTCTGGCTGCCGGCTTCGCCTTACTGGGCATCTCCTTCTTTGCCCTGC
CTGCCGGCATCCTGGGCTCTGGCTTTGCCCTGAAGGTCCAGGAGCAGCACAGGCAGAAGCACTT
TGAGAAGCGCAGGATGCCAGCAGCTAATCTCATCCAGGCTGCGTGGCGCCTGTACTCCACGGAC
ACAAGCCGGGCCTATTTGACAGCCACCTGGTATTACTATGACAGCATTCTCCCATCTTTCAGAG
AGCTGGCCCTCTTGTTTGAGCACATACAACGGGCCCGCAATGGGGGCTTACGGCCCCTGGAGGT
GAGGCGGGCGCCAGTACCTGATGGAGCGCCCTCTCGCTACCCGCCCGTTGCCACCTGCCACCGG
CCAGGCAGTGCCTCCTTCTGCCCTGGGGAAAGCAGCCGGATGGGCATCAAAGACCGAATCCGCA
TAAGCAGCTCCCAGAAGCGGACAGGCCCCTCCAAGCAGCATCTGGCCCCTCCGCCGATTCCCAC
CTCGCCAAGCAGTGAGCAGGTGGGCGAGGCATCCAGCCCCAGCAAGGTGCAGAAAAGCTGGAGC
TTCAATGACCGCACCCGCTTCCGGGCCTCTCTAAGACTCAAGCCTCGCTGCTCTGCTGAGGAGG
GCCCCTCAGAGGAAGTGGCCGAGGAGAAGAGCTACCAGTGTGAGCTTACGGTGGATGATGTCAT
GCCTGCTGTGAAGACGGTCATCCGTTCTGTCAGGATTCTGAAGTTCCTGGTAGCCAAAAGGAAA
TTCAAGGAGACGCTGCGGCCATATGATGTGAAGGATGTCATTGAGCAGTATTCAGCAGGACACC
TGGACATGCTGGGACGAATCAAGAGCCTGCAAGCCCGGGTGGACCAAATTGTGGGTCGGGGCCC
TGGGGACCGCAAGACTCGGGAGAAGGGCGATAAGGGTCCTTCAGACACGGAGGCAGTGGATGAA
ATCAGCATGATGGGGCGCGTAGTCAAGGTGGAAAAGCAGGTGCAGTCCATCGAACATAAGTTGG
ATCTGCTGCTGGGCTTCTACTCGCGCTGCCTCCGCTCTGGCACCTCGGCCAGCCTGGGCACTGT
GCAAGTGCCGCTCTTCGACCCCGACATCACCTCGGACTACCACAGCCCTGTAGACCACGAGGAC
ATCTCTGTCTCCGCACAGACGCTCAGCATCTCTCGCTCAGTCAGCACCAACATGGACGGATCCC
GGGCTGACTACAAAGACCATGACGGTGATTATAAAGATCATGACATCGACTACAAGGATGACGA
TGACAAGGGCTCCGGAGAGGGCAGAGGAAGTCTGCTAACATGCGGTGACGTCGAGGAGAATCCT
GGCCCAATGGTGAGCAAGGGCGAGGCAGTGATCAAGGAGTTCATGCGGTTCAAGGTGCACATGG
AGGGCTCCATGAACGGCCACGAGTTCGAGATCGAGGGCGAGGGCGAGGGCCGCCCCTACGAGGG
CACCCAGACCGCCAAGCTGAAGGTGACCAAGGGTGGCCCCCTGCCCTTCTCCTGGGACATCCTG
TCCCCTCAGTTCATGTACGGCTCCAGGGCCTTCATCAAGCACCCCGCCGACATCCCCGACTACT
ATAAGCAGTCCTTCCCCGAGGGCTTCAAGTGGGAGCGCGTGATGAACTTCGAGGACGGCGGCGC
CGTGACCGTGACCCAGGACACCTCCCTGGAGGACGGCACCCTGATCTACAAGGTGAAGCTCCGC
GGCACCAACTTCCCTCCTGACGGCCCCGTAATGCAGAAGAAGACAATGGGCTGGGAAGCGTCCA
CCGAGCGGTTGTACCCCGAGGACGGCGTGCTGAAGGGCGACATTAAGATGGCCCTGCGCCTGAA
GGACGGCGGCCGCTACCTGGCGGACTTCAAGACCACCTACAAGGCCAAGAAGCCCGTGCAGATG
CCCGGCGCCTACAACGTCGACCGCAAGTTGGACATCACCTCCCACAACGAGGACTACACCGTGG
TGGAACAGTACGAACGCTCCGAGGGCCGCCACTCCACCGGCGGCATGGACGAGCTGTACAAGTA
ATAAGAGCTCGCTGATCAGCCTCGACTGTGCCTTCTAGTTGCCAGCCATCTGTTGTTTGCCCCT
CCCCCGTGCCTTCCTTGACCCTGGAAGGTGCCACTCCCACTGTCCTTTCCTAATAAAATGAGGA
AATTGCATCGCATTGTCTGAGTAGGTGTCATTCTATTCTGGGGGGTGGGGTGGGGCAGGACAGC
AAGGGGGAGGATTGGGAAGACAATAGCAGGCATGCTGGGGATGCGGTGGGCTCTATGGAAGCTT
GAATTCAGCTGACGTGCCTCGGACCGCTAGGAACCCCTAGTGATGGAGTTGGCCACTCCCTCTC
TGCGCGCTCGCTCGCTCACTGAGGCCGGGCGACCAAAGGTCGCCCGACGCCCGGGCTTTGCCCG
GGCGGCCTCAGTGAGCGAGCGAGCGCGCAGCTGCCTGCAGG

Exemplary U6shRNA-hKCNQ4 Construct Sequences

Exemplary LucT2AGFP-U6shRNA-hKCNQ4 without a specific shRNA sequence
(SEQ ID NO: 258)
CTGCGCGCTCGCTCGCTCACTGAGGCCGCCCGGGCGTCGGGCGACCTTTGGTCGCCCGGCCTCA
GTGAGCGAGCGAGCGCGCAGAGAGGGAGTGGCCAACTCCATCACTAGGGGTTCCTGCGGCCGCA
CGCGTCTAGATCCCATATATGGAGTTCCGCGTTACATAACTTACGGTAAATGGCCCGCCTGGCT
GACCGCCCAACGACCCCCGCCCATTGACGTCAATAATGACGTATGTTCCCATAGTAACGCCAAT
AGGGACTTTCCATTGACGTCAATGGGTGGAGTATTTACGGTAAACTGCCCACTTGGCAGTACAT
CAAGTGTATCATATGCCAAGTACGCCCCCTATTGACGTCAATGACGGTAAATGGCCCGCCTGGC
ATTATGCCCAGTACATGACCTTATGGGACTTTCCTACTTGGCAGTACATCTACGTATTAGTCAT
CGCTATTACCATGGTGATGCGGTTTTGGCAGTACATCAATGGGCGTGGATAGCGGTTTGACTCA
CGGGGATTTCCAAGTCTCCACCCCATTGACGTCAATGGGAGTTTGTTTTGGCACCAAAATCAAC
GGGACTTTCCAAAATGTCGTAACAACTCCGCCCCATTGACGCAAATGGGCGGTAGGCGTGTACG
GTGGGAGGTCTATATAAGCAGAGCTCGTTTAGTGAACCGTCAGATCGCCTGGAGACGCACCGGT
GCCACCATGGAAGATGCCAAAAACATTAAGAAGGGCCCAGCGCCATTCTACCCACTCGAAGACG
GGACCGCCGGCGAGCAGCTGCACAAAGCCATGAAGCGCTACGCCCTGGTGCCCGGCACCATCGC
CTTTACCGACGCACATATCGAGGTGGACATTACCTACGCCGAGTACTTCGAGATGAGCGTTCGG
CTGGCAGAAGCTATGAAGCGCTATGGGCTGAATACAAACCATCGGATCGTGGTGTGCAGCGAGA
ATAGCTTGCAGTTCTTCATGCCCGTGTTGGGTGCCCTGTTCATCGGTGTGGCTGTGGCCCCAGC
TAACGACATCTACAACGAGCGCGAGCTGCTGAACAGCATGGGCATCAGCCAGCCCACCGTCGTA
TTCGTGAGCAAGAAAGGGCTGCAAAAGATCCTCAACGTGCAAAAGAAGCTACCGATCATACAAA
AGATCATCATCATGGATAGCAAGACCGACTACCAGGGCTTCCAAAGCATGTACACCTTCGTGAC
TTCCCATTTGCCACCCGGCTTCAACGAGTACGACTTCGTGCCCGAGAGCTTCGACCGGGACAAA
ACCATCGCCCTGATCATGAACAGTAGTGGCAGTACCGGATTGCCCAAGGGCGTAGCCCTACCGC
ACCGCACCGCTTGTGTCCGATTCAGTCATGCCCGCGACCCCATCTTCGGCAACCAGATCATCCC
CGACACCGCTATCCTCAGCGTGGTGCCATTTCACCACGGCTTCGGCATGTTCACCACGCTGGGC
TACTTGATCTGCGGCTTTCGGGTCGTGCTCATGTACCGCTTCGAGGAGGAGCTATTCTTGCGCA
GCTTGCAAGACTATAAGATTCAATCTGCCCTGCTGGTGCCCACACTATTTAGCTTCTTCGCTAA
GAGCACTCTCATCGACAAGTACGACCTAAGCAACTTGCACGAGATCGCCAGCGGCGGGGCGCCG
CTCAGCAAGGAGGTAGGTGAGGCCGTGGCCAAACGCTTCCACCTACCAGGCATCCGCCAGGGCT
ACGGCCTGACAGAAACAACCAGCGCCATTCTGATCACCCCCGAAGGGGACGACAAGCCTGGCGC
AGTAGGCAAGGTGGTGCCCTTCTTCGAGGCTAAGGTGGTGGACTTGGACACAGGTAAGACACTG
GGTGTGAACCAGCGCGGCGAGCTGTGCGTCCGTGGCCCCATGATCATGAGCGGCTACGTTAACA
ACCCCGAGGCTACAAACGCTCTCATCGACAAGGACGGCTGGCTGCACAGCGGCGACATCGCCTA
CTGGGACGAGGACGAGCACTTCTTCATCGTGGACCGGCTGAAGAGCCTGATCAAATACAAGGGC
TACCAGGTAGCCCCAGCCGAACTGGAGAGCATCCTGCTGCAACACCCCAACATCTTCGACGCCG
GGGTCGCCGGCCTGCCCGACGACGATGCCGGCGAGCTGCCCGCCGCAGTCGTCGTGCTGGAACA
CGGTAAAACCATGACCGAGAAGGAGATCGTGGACTATGTGGCCAGCCAGGTTACAACCGCCAAG
AAGCTGCGCGGTGGTGTTGTGTTCGTGGACGAGGTGCCTAAAGGACTGACCGGCAAGTTGGACG
CCCGCAAGATCCGCGAGATTCTCATTAAGGCCAAGAAGGGCGGCAAGATCGCCGTGGGCTCCGG
AGAGGGCAGAGGAAGTCTGCTAACATGCGGTGACGTCGAGGAGAATCCTGGCCCAATGGAGAGC
GACGAGAGCGGCCTGCCCGCCATGGAGATCGAGTGCCGCATCACCGGCACCCTGAACGGCGTGG
AGTTCGAGCTGGTGGGCGGCGGAGAGGGCACCCCCGAGCAGGGCCGCATGACCAACAAGATGAA
GAGCACCAAAGGCGCCCTGACCTTCAGCCCCTACCTGCTGAGCCACGTGATGGGCTACGGCTTC
TACCACTTCGGCACCTACCCCAGCGGCTACGAGAACCCCTTCCTGCACGCCATCAACAACGGCG
GCTACACCAACACCCGCATCGAGAAGTACGAGGACGGCGGCGTGCTGCACGTGAGCTTCAGCTA
CCGCTACGAGGCCGGCCGCGTGATCGGCGACTTCAAGGTGATGGGCACCGGCTTCCCCGAGGAC
AGCGTGATCTTCACCGACAAGATCATCCGCAGCAACGCCACCGTGGAGCACCTGCACCCCATGG
GCGATAACGATCTGGATGGCAGCTTCACCCGCACCTTCAGCCTGCGCGACGGCGGCTACTACAG
CTCCGTGGTGGACAGCCACATGCACTTCAAGAGCGCCATCCACCCCAGCATCCTGCAGAACGGG
GGCCCCATGTTCGCCTTCCGCCGCGTGGAGGAGGATCACAGCAACACCGAGCTGGGCATCGTGG
AGTACCAGCACGCCTTCAAGACCCCGGATGCAGATGCCGGTGAAGAATAAGAGCTCGCTGATCA
GCCTCGATAAGATACATTGATGAGTTTGGAGAAACCACAACTAGAATGCAGTGAAAAAAATGCT
TTATTTGTGAAATTTGTGATGCTATTGCTTTATTTGTAACCATTATAAGCTGCAATAAACAAGT
TTTAATTAAGGTCGGGCAGGAAGAGGGCCTATTTCCCATGATTCCTTCATATTTGCATATACGA
TACAAGGCTGTTAGAGAGATAATTAGAATTAATTTGAGTGTAAACACAAAGATATTAGTAGAAA
ATACGTGACGTAGAAAGTAATAATTTCTTGGGTAGTTTGCAGTTTTAAAATTATGTTTTAAAAT
GGACTATCATATGCTTACCGTAACTTGAAAGTATTTCGATTTCTTGGCTTTATATATCTTGTGG
AAAGGACGAAACACCGNNNNNTTTTTTAAGCTTTTTTGAATTCAGCTGACGTGCCTCGGACCGC
TAGGAACCCCTAGTGATGGAGTTGGCCACTCCCTCTCTGCGCGCTCGCTCGCTCACTGAGGCCG
GGCGACCAAAGGTCGCCCGACGCCCGGGCTTTGCCCGGGCGGCCTCAGTGAGCGAGCGAGCGCG
CAG
Exemplary pITR.CMV.LucT2AGFP-U6shRNA-hKCNQ4-395 sequence
(SEQ ID NO: 259)
CTGCGCGCTCGCTCGCTCACTGAGGCCGCCCGGGCGTCGGGCGACCTTTGGTCGCCCGGCCTCA
GTGAGCGAGCGAGCGCGCAGAGAGGGAGTGGCCAACTCCATCACTAGGGGTTCCTGCGGCCGCA
CGCGTCTAGATCCCATATATGGAGTTCCGCGTTACATAACTTACGGTAAATGGCCCGCCTGGCT
GACCGCCCAACGACCCCCGCCCATTGACGTCAATAATGACGTATGTTCCCATAGTAACGCCAAT
AGGGACTTTCCATTGACGTCAATGGGTGGAGTATTTACGGTAAACTGCCCACTTGGCAGTACAT
CAAGTGTATCATATGCCAAGTACGCCCCCTATTGACGTCAATGACGGTAAATGGCCCGCCTGGC
ATTATGCCCAGTACATGACCTTATGGGACTTTCCTACTTGGCAGTACATCTACGTATTAGTCAT
CGCTATTACCATGGTGATGCGGTTTTGGCAGTACATCAATGGGCGTGGATAGCGGTTTGACTCA
CGGGGATTTCCAAGTCTCCACCCCATTGACGTCAATGGGAGTTTGTTTTGGCACCAAAATCAAC
GGGACTTTCCAAAATGTCGTAACAACTCCGCCCCATTGACGCAAATGGGCGGTAGGCGTGTACG
GTGGGAGGTCTATATAAGCAGAGCTCGTTTAGTGAACCGTCAGATCGCCTGGAGACGCACCGGT
GCCACCATGGAAGATGCCAAAAACATTAAGAAGGGCCCAGCGCCATTCTACCCACTCGAAGACG
GGACCGCCGGCGAGCAGCTGCACAAAGCCATGAAGCGCTACGCCCTGGTGCCCGGCACCATCGC
CTTTACCGACGCACATATCGAGGTGGACATTACCTACGCCGAGTACTTCGAGATGAGCGTTCGG
CTGGCAGAAGCTATGAAGCGCTATGGGCTGAATACAAACCATCGGATCGTGGTGTGCAGCGAGA
ATAGCTTGCAGTTCTTCATGCCCGTGTTGGGTGCCCTGTTCATCGGTGTGGCTGTGGCCCCAGC
TAACGACATCTACAACGAGCGCGAGCTGCTGAACAGCATGGGCATCAGCCAGCCCACCGTCGTA
TTCGTGAGCAAGAAAGGGCTGCAAAAGATCCTCAACGTGCAAAAGAAGCTACCGATCATACAAA
AGATCATCATCATGGATAGCAAGACCGACTACCAGGGCTTCCAAAGCATGTACACCTTCGTGAC
TTCCCATTTGCCACCCGGCTTCAACGAGTACGACTTCGTGCCCGAGAGCTTCGACCGGGACAAA
ACCATCGCCCTGATCATGAACAGTAGTGGCAGTACCGGATTGCCCAAGGGCGTAGCCCTACCGC
ACCGCACCGCTTGTGTCCGATTCAGTCATGCCCGCGACCCCATCTTCGGCAACCAGATCATCCC
CGACACCGCTATCCTCAGCGTGGTGCCATTTCACCACGGCTTCGGCATGTTCACCACGCTGGGC
TACTTGATCTGCGGCTTTCGGGTCGTGCTCATGTACCGCTTCGAGGAGGAGCTATTCTTGCGCA
GCTTGCAAGACTATAAGATTCAATCTGCCCTGCTGGTGCCCACACTATTTAGCTTCTTCGCTAA
GAGCACTCTCATCGACAAGTACGACCTAAGCAACTTGCACGAGATCGCCAGCGGCGGGGCGCCG
CTCAGCAAGGAGGTAGGTGAGGCCGTGGCCAAACGCTTCCACCTACCAGGCATCCGCCAGGGCT
ACGGCCTGACAGAAACAACCAGCGCCATTCTGATCACCCCCGAAGGGGACGACAAGCCTGGCGC
AGTAGGCAAGGTGGTGCCCTTCTTCGAGGCTAAGGTGGTGGACTTGGACACAGGTAAGACACTG
GGTGTGAACCAGCGCGGCGAGCTGTGCGTCCGTGGCCCCATGATCATGAGCGGCTACGTTAACA
ACCCCGAGGCTACAAACGCTCTCATCGACAAGGACGGCTGGCTGCACAGCGGCGACATCGCCTA
CTGGGACGAGGACGAGCACTTCTTCATCGTGGACCGGCTGAAGAGCCTGATCAAATACAAGGGC
TACCAGGTAGCCCCAGCCGAACTGGAGAGCATCCTGCTGCAACACCCCAACATCTTCGACGCCG
GGGTCGCCGGCCTGCCCGACGACGATGCCGGCGAGCTGCCCGCCGCAGTCGTCGTGCTGGAACA
CGGTAAAACCATGACCGAGAAGGAGATCGTGGACTATGTGGCCAGCCAGGTTACAACCGCCAAG
AAGCTGCGCGGTGGTGTTGTGTTCGTGGACGAGGTGCCTAAAGGACTGACCGGCAAGTTGGACG
CCCGCAAGATCCGCGAGATTCTCATTAAGGCCAAGAAGGGCGGCAAGATCGCCGTGGGCTCCGG
AGAGGGCAGAGGAAGTCTGCTAACATGCGGTGACGTCGAGGAGAATCCTGGCCCAATGGAGAGC
GACGAGAGCGGCCTGCCCGCCATGGAGATCGAGTGCCGCATCACCGGCACCCTGAACGGCGTGG
AGTTCGAGCTGGTGGGCGGCGGAGAGGGCACCCCCGAGCAGGGCCGCATGACCAACAAGATGAA
GAGCACCAAAGGCGCCCTGACCTTCAGCCCCTACCTGCTGAGCCACGTGATGGGCTACGGCTTC
TACCACTTCGGCACCTACCCCAGCGGCTACGAGAACCCCTTCCTGCACGCCATCAACAACGGCG
GCTACACCAACACCCGCATCGAGAAGTACGAGGACGGCGGCGTGCTGCACGTGAGCTTCAGCTA
CCGCTACGAGGCCGGCCGCGTGATCGGCGACTTCAAGGTGATGGGCACCGGCTTCCCCGAGGAC
AGCGTGATCTTCACCGACAAGATCATCCGCAGCAACGCCACCGTGGAGCACCTGCACCCCATGG
GCGATAACGATCTGGATGGCAGCTTCACCCGCACCTTCAGCCTGCGCGACGGCGGCTACTACAG
CTCCGTGGTGGACAGCCACATGCACTTCAAGAGCGCCATCCACCCCAGCATCCTGCAGAACGGG
GGCCCCATGTTCGCCTTCCGCCGCGTGGAGGAGGATCACAGCAACACCGAGCTGGGCATCGTGG
AGTACCAGCACGCCTTCAAGACCCCGGATGCAGATGCCGGTGAAGAATAAGAGCTCGCTGATCA
GCCTCGATAAGATACATTGATGAGTTTGGACAAACCACAACTAGAATGCAGTGAAAAAAATGCT
TTATTTGTGAAATTTGTGATGCTATTGCTTTATTTGTAACCATTATAAGCTGCAATAAACAAGT
TTTAATTAAGGTCGGGCAGGAAGAGGGCCTATTTCCCATGATTCCTTCATATTTGCATATACGA
TAGAAGGCTGTTAGAGAGATAATTAGAATTAATTTGACTGTAAACACAAAGATATTAGTAGAAA
ATACGTGACGTAGAAAGTAATAATTTCTTGGGTAGTTTGCAGTTTTAAAATTATGTTTTAAAAT
GGACTATCATATGCTTACCGTAACTTGAAAGTATTTCGATTTCTTGGCTTTATATATCTTGTGG
AAAGGACGAAACACCGTCCTCATCTTGGAATTCGTGATGCGAACATCACGAATTCCAAGATGAG
GATTTTTTAAGCTTTTTTGAATTCAGCTGACGTGCCTCGGACCGCTAGGAACCCCTAGTGATGG
AGTTGGCCACTCCCTCTCTGCGCGCTCGCTCGCTCACTGAGGCCGGGCGACCAAAGGTCGCCCG
ACGCCCGGGCTTTGCCCGGGCGGCCTCAGTGAGCGAGCGAGCGCGCAG
Exemplary pITR.CMV.LucT2AGFP-U6shRNA-hKCNQ4-909 sequence
(SEQ ID NO: 260)
CTGCGCGCTCGCTCGCTCACTGAGGCCGCCCGGGCGTCGGGCGACCTTTGGTCGCCCGGCCTCA
GTGAGCGAGCGAGCGCGCAGAGAGGGAGTGGCCAACTCCATCACTAGGGGTTCCTGCGGCCGCA
CGCGTCTAGATCCCATATATGGAGTTCCGCGTTACATAACTTACGGTAAATGGCCCGCCTGGCT
GACCGCCCAACGACCCCCGCCCATTGACGTCAATAATGACGTATGTTCCCATAGTAACGCCAAT
AGGGACTTTCCATTGACGTCAATGGGTGGAGTATTTACGGTAAACTGCCCACTTGGCAGTACAT
CAAGTGTATCATATGCCAAGTACGCCCCCTATTGACGTCAATGACGGTAAATGGCCCGCCTGGC
ATTATGCCCAGTACATGACCTTATGGGACTTTCCTACTTGGCAGTACATCTACGTATTAGTCAT
CGCTATTACCATGGTGATGCGGTTTTGGCAGTACATCAATGGGCGTGGATAGCGGTTTGACTCA
CGGGGATTTCCAAGTCTCCACCCCATTGACGTCAATGGGAGTTTGTTTTGGCACCAAAATCAAC
GGGACTTTCCAAAATGTCGTAACAACTCCGCCCCATTGACGCAAATGGGCGGTAGGCGTGTACG
GTGGGAGGTCTATATAAGCAGAGCTCGTTTAGTGAACCGTcagatcgcctggagacgcACCGGT
GCCACCATGGAAGATGCCAAAAACATTAAGAAGGGCCCAGCGCCATTCTACCCACTCGAAGACG
GGACCGCCGGCGAGCAGCTGCACAAAGCCATGAAGCGCTACGCCCTGGTGCCCGGCACCATCGC
CTTTACCGACGCACATATCGAGGTGGACATTACCTACGCCGAGTACTTCGAGATGAGCGTTCGG
CTGGCAGAAGCTATGAAGCGCTATGGGCTGAATACAAACCATCGGATCGTGGTGTGCAGCGAGA
ATAGCTTGCAGTTCTTCATGCCCGTGTTGGGTGCCCTGTTCATCGGTGTGGCTGTGGCCCCAGC
TAACGACATCTACAACGAGCGCGAGCTGCTGAACAGCATGGGCATCAGCCAGCCCACCGTCGTA
TTCGTGAGCAAGAAAGGGCTGCAAAAGATCCTCAACGTGCAAAAGAAGCTACCGATCATACAAA
AGATCATCATCATGGATAGCAAGACCGACTACCAGGGCTTCCAAAGCATGTACACCTTCGTGAC
TTCCCATTTGCCACCCGGCTTCAACGAGTACGACTTCGTGCCCGAGAGCTTCGACCGGGACAAA
ACCATCGCCCTGATCATGAACAGTAGTGGCAGTACCGGATTGCCCAAGGGCGTAGCCCTACCGC
ACCGCACCGCTTGTGTCCGATTCAGTCATGCCCGCGACCCCATCTTCGGCAACCAGATCATCCC
CGACACCGCTATCCTCAGCGTGGTGCCATTTCACCACGGCTTCGGCATGTTCACCACGCTGGGC
TACTTGATCTGCGGCTTTCGGGTCGTGCTCATGTACCGCTTCGAGGAGGAGCTATTCTTGCGCA
GCTTGCAAGACTATAAGATTCAATCTGCCCTGCTGGTGCCCACACTATTTAGCTTCTTCGCTAA
GAGCACTCTCATCGACAAGTACGACCTAAGCAACTTGCACGAGATCGCCAGCGGCGGGGCGCCG
CTCAGCAAGGAGGTAGGTGAGGCCGTGGCCAAACGCTTCCACCTACCAGGCATCCGCCAGGGCT
ACGGCCTGACAGAAACAACCAGCGCCATTCTGATCACCCCCGAAGGGGACGACAAGCCTGGCGC
AGTAGGCAAGGTGGTGCCCTTCTTCGAGGCTAAGGTGGTGGACTTGGACACAGGTAAGACACTG
GGTGTGAACCAGCGCGGCGAGCTGTGCGTCCGTGGCCCCATGATCATGAGCGGCTACGTTAACA
ACCCCGAGGCTACAAACGCTCTCATCGACAAGGACGGCTGGCTGCACAGCGGCGACATCGCCTA
CTGGGACGAGGACGAGCACTTCTTCATCGTGGACCGGCTGAAGAGCCTGATCAAATACAAGGGC
TACCAGGTAGCCCCAGCCGAACTGGAGAGCATCCTGCTGCAACACCCCAACATCTTCGACGCCG
GGGTCGCCGGCCTGCCCGACGACGATGCCGGCGAGCTGCCCGCCGCAGTCGTCGTGCTGGAACA
CGGTAAAACCATGACCGAGAAGGAGATCGTGGACTATGTGGCCAGCCAGGTTACAACCGCCAAG
AAGCTGCGCGGTGGTGTTGTGTTCGTGGACGAGGTGCCTAAAGGACTGACCGGCAAGTTGGACG
CCCGCAAGATCCGCGAGATTCTCATTAAGGCCAAGAAGGGCGGCAAGATCGCCGTGGGCTCCGG
AGAGGGCAGAGGAAGTCTGCTAACATGCGGTGACGTCGAGGAGAATCCTGGCCCAATGGAGAGC
GACGAGAGCGGCCTGCCCGCCATGGAGATCGAGTGCCGCATCACCGGCACCCTGAACGGCGTGG
AGTTCGAGCTGGTGGGCGGCGGAGAGGGCACCCCCGAGCAGGGCCGCATGACCAACAAGATGAA
GAGCACCAAAGGCGCCCTGACCTTCAGCCCCTACCTGCTGAGCCACGTGATGGGCTACGGCTTC
TACCACTTCGGCACCTACCCCAGCGGCTACGAGAACCCCTTCCTGCACGCCATCAACAACGGCG
GCTACACCAACACCCGCATCGAGAAGTACGAGGACGGCGGCGTGCTGCACGTGAGCTTCAGCTA
CCGCTACGAGGCCGGCCGCGTGATCGGCGACTTCAAGGTGATGGGCACCGGCTTCCCCGAGGAC
AGCGTGATCTTCACCGACAAGATCATCCGCAGCAACGCCACCGTGGAGCACCTGCACCCCATGG
GCGATAACGATCTGGATGGCAGCTTCACCCGCACCTTCAGCCTGCGCGACGGCGGCTACTACAG
CTCCGTGGTGGACAGCCACATGCACTTCAAGAGCGCCATCCACCCCAGCATCCTGCAGAACGGG
GGCCCCATGTTCGCCTTCCGCCGCGTGGAGGAGGATCACAGCAACACCGAGCTGGGCATCGTGG
AGTACCAGCACGCCTTCAAGACCCCGGATGCAGATGCCGGTGAAGAATAAGAGCTCGCTGATCA
GCCTCGATAAGATACATTGATGAGTTTGGAGAAACCACAACTAGAATGCAGTGAAAAAAATGCT
TTATTTGTGAAATTTGTGATGCTATTGCTTTATTTGTAACCATTATAAGCTGCAATAAACAAGT
TTTAATTAAGGTCGGGCAGGAAGAGGGCCTATTTCCCATGATTCCTTCATATTTGCATATACGA
TAGAAGGCTGTTAGAGAGATAATTAGAATTAATTTGACTGTAAACACAAAGATATTAGTAGAAA
ATACGTGACGTAGAAAGTAATAATTTCTTGGGTAGTTTGCAGTTTTAAAATTATGTTTTAAAAT
GGACTATCATATGCTTACCGTAACTTGAAAGTATTTCGATTTCTTGGCTTTATATATCTTGTGG
AAAGGACGAAACACCGCGCCTTACTGGGCATCTCTTTCTCGAAAGAAAGAGATGCCCAGTAAGG
CGTTTTTTAAGCTTTTTTGAATTCAGCTGACGTGCCTCGGACCGCTAGGAACCCCTAGTGATGG
AGTTGGCCACTCCCTCTCTGCGCGCTCGCTCGCTCACTGAGGCCGGGCGACCAAAGGTCGCCCG
ACGCCCGGGCTTTGCCCGGGCGGCCTCAGTGAGCGAGCGAGCGCGCAG
Exemplary pITR.CMV.LucT2AGFP-U6shRNA-hKCNQ4-1095 sequence
(SEQ ID NO: 261)
CTGCGCGCTCGCTCGCTCACTGAGGCCGCCCGGGCGTCGGGCGACCTTTGGTCGCCCGGCCTCA
GTGAGCGAGCGAGCGCGCAGAGAGGGAGTGGCCAACTCCATCACTAGGGGTTCCTGCGGCCGCA
CGCGTCTAGATCCCATATATGGAGTTCCGCGTTACATAACTTACGGTAAATGGCCCGCCTGGCT
GACCGCCCAACGACCCCCGCCCATTGACGTCAATAATGACGTATGTTCCCATAGTAACGCCAAT
AGGGACTTTCCATTGACGTCAATGGGTGGAGTATTTACGGTAAACTGCCCACTTGGCAGTACAT
CAAGTGTATCATATGCCAAGTACGCCCCCTATTGACGTCAATGACGGTAAATGGCCCGCCTGGC
ATTATGCCCAGTACATGACCTTATGGGACTTTCCTACTTGGCAGTACATCTACGTATTAGTCAT
CGCTATTACCATGGTGATGCGGTTTTGGCAGTACATCAATGGGCGTGGATAGCGGTTTGACTCA
CGGGGATTTCCAAGTCTCCACCCCATTGACGTCAATGGGAGTTTGTTTTGGCACCAAAATCAAC
GGGACTTTCCAAAATGTCGTAACAACTCCGCCCCATTGACGCAAATGGGCGGTAGGCGTGTACG
GTGGGAGGTCTATATAAGCAGAGCTCGTTTAGTGAACCGTcagatcgcctggagacgcACCGGT
GCCACCATGGAAGATGCCAAAAACATTAAGAAGGGCCCAGCGCCATTCTACCCACTCGAAGACG
GGACCGCCGGCGAGCAGCTGCACAAAGCCATGAAGCGCTACGCCCTGGTGCCCGGCACCATCGC
CTTTACCGACGCACATATCGAGGTGGACATTACCTACGCCGAGTACTTCGAGATGAGCGTTCGG
CTGGCAGAAGCTATGAAGCGCTATGGGCTGAATACAAACCATCGGATCGTGGTGTGCAGCGAGA
ATAGCTTGCAGTTCTTCATGCCCGTGTTGGGTGCCCTGTTCATCGGTGTGGCTGTGGCCCCAGC
TAACGACATCTACAACGAGCGCGAGCTGCTGAACAGCATGGGCATCAGCCAGCCCACCGTCGTA
TTCGTGAGCAAGAAAGGGCTGCAAAAGATCCTCAACGTGCAAAAGAAGCTACCGATCATACAAA
AGATCATCATCATGGATAGCAAGACCGACTACCAGGGCTTCCAAAGCATGTACACCTTCGTGAC
TTCCCATTTGCCACCCGGCTTCAACGAGTACGACTTCGTGCCCGAGAGCTTCGACCGGGACAAA
ACCATCGCCCTGATCATGAACAGTAGTGGCAGTACCGGATTGCCCAAGGGCGTAGCCCTACCGC
ACCGCACCGCTTGTGTCCGATTCAGTCATGCCCGCGACCCCATCTTCGGCAACCAGATCATCCC
CGACACCGCTATCCTCAGCGTGGTGCCATTTCACCACGGCTTCGGCATGTTCACCACGCTGGGC
TACTTGATCTGCGGCTTTCGGGTCGTGCTCATGTACCGCTTCGAGGAGGAGCTATTCTTGCGCA
GCTTGCAAGACTATAAGATTCAATCTGCCCTGCTGGTGCCCACACTATTTAGCTTCTTCGCTAA
GAGCACTCTCATCGACAAGTACGACCTAAGCAACTTGCACGAGATCGCCAGCGGCGGGGCGCCG
CTCAGCAAGGAGGTAGGTGAGGCCGTGGCCAAACGCTTCCACCTACCAGGCATCCGCCAGGGCT
ACGGCCTGACAGAAACAACCAGCGCCATTCTGATCACCCCCGAAGGGGACGACAAGCCTGGCGC
AGTAGGCAAGGTGGTGCCCTTCTTCGAGGCTAAGGTGGTGGACTTGGACACAGGTAAGACACTG
GGTGTGAACCAGCGCGGCGAGCTGTGCGTCCGTGGCCCCATGATCATGAGCGGCTACGTTAACA
ACCCCGAGGCTACAAACGCTCTCATCGACAAGGACGGCTGGCTGCACAGCGGCGACATCGCCTA
CTGGGACGAGGACGAGCACTTCTTCATCGTGGACCGGCTGAAGAGCCTGATCAAATACAAGGGC
TACCAGGTAGCCCCAGCCGAACTGGAGAGCATCCTGCTGCAACACCCCAACATCTTCGACGCCG
GGGTCGCCGGCCTGCCCGACGACGATGCCGGCGAGCTGCCCGCCGCAGTCGTCGTGCTGGAACA
CGGTAAAACCATGACCGAGAAGGAGATCGTGGACTATGTGGCCAGCCAGGTTACAACCGCCAAG
AAGCTGCGCGGTGGTGTTGTGTTCGTGGACGAGGTGCCTAAAGGACTGACCGGCAAGTTGGACG
CCCGCAAGATCCGCGAGATTCTCATTAAGGCCAAGAAGGGCGGCAAGATCGCCGTGGGCTCCGG
AGAGGGCAGAGGAAGTCTGCTAACATGCGGTGACGTCGAGGAGAATCCTGGCCCAATGGAGAGC
GACGAGAGCGGCCTGCCCGCCATGGAGATCGAGTGCCGCATCACCGGCACCCTGAACGGCGTGG
AGTTCGAGCTGGTGGGCGGCGGAGAGGGCACCCCCGAGCAGGGCCGCATGACCAACAAGATGAA
GAGCACCAAAGGCGCCCTGACCTTCAGCCCCTACCTGCTGAGCCACGTGATGGGCTACGGCTTC
TACCACTTCGGCACCTACCCCAGCGGCTACGAGAACCCCTTCCTGCACGCCATCAACAACGGCG
GCTACACCAACACCCGCATCGAGAAGTACGAGGACGGCGGCGTGCTGCACGTGAGCTTCAGCTA
CCGCTACGAGGCCGGCCGCGTGATCGGCGACTTCAAGGTGATGGGCACCGGCTTCCCCGAGGAC
AGCGTGATCTTCACCGACAAGATCATCCGCAGCAACGCCACCGTGGAGCACCTGCACCCCATGG
GCGATAACGATCTGGATGGCAGCTTCACCCGCACCTTCAGCCTGCGCGACGGCGGCTACTACAG
CTCCGTGGTGGACAGCCACATGCACTTCAAGAGCGCCATCCACCCCAGCATCCTGCAGAACGGG
GGCCCCATGTTCGCCTTCCGCCGCGTGGAGGAGGATCACAGCAACACCGAGCTGGGCATCGTGG
AGTACCAGCACGCCTTCAAGACCCCGGATGCAGATGCCGGTGAAGAATAAGAGCTCGCTGATCA
GCCTCGATAAGATACATTGATGAGTTTGGACAAACCACAACTAGAATGCAGTGAAAAAAATGCT
TTATTTGTGAAATTTGTGATGCTATTGCTTTATTTGTAACCATTATAAGCTGCAATAAACAAGT
TTTAATTAAGGTCGGGCAGGAAGAGGGCCTATTTCCCATGATTCCTTCATATTTGCATATACGA
TAGAAGGCTGTTAGAGAGATAATTAGAATTAATTTGACTGTAAACACAAAGATATTAGTAGAAA
ATACGTGACGTAGAAAGTAATAATTTCTTGGGTAGTTTGCAGTTTTAAAATTATGTTTTAAAAT
GGACTATCATATGCTTACCGTAACTTGAAAGTATTTCGATTTCTTGGCTTTATATATCTTGTGG
AAAGGACGAAACACCGCTGGTACTACTATGACAGTATCGAAATACTGTCATAGTAGTAGCAGTT
TTTTAAGCTTTTTTGAATTCAGCTGACGTGCCTCGGACCGCTAGGAACCCCTAGTGATGGAGTT
GGCCACTCCCTCTCTGCGCGCTCGCTCGCTCACTGAGGCCGGGCGACCAAAGGTCGCCCGACGC
CCGGGCTTTGCCCGGGCGGCCTCAGTGAGCGAGCGAGCGCGCAG
Exemplary pITR.CMV.LucT2AGFP-U6shRNA-hKCNQ4-1531 sequence
(SEQ ID NO: 262)
CTGCGCGCTCGCTCGCTCACTGAGGCCGCCCGGGCGTCGGGCGACCTTTGGTCGCCCGGCCTCA
GTGAGCGAGCGAGCGCGCAGAGAGGGAGTGGCCAACTCCATCACTAGGGGTTCCTGCGGCCGCA
CGCGTCTAGATCCCATATATGGAGTTCCGCGTTACATAACTTACGGTAAATGGCCCGCCTGGCT
GACCGCCCAACGACCCCCGCCCATTGACGTCAATAATGACGTATGTTCCCATAGTAACGCCAAT
AGGGACTTTCCATTGACGTCAATGGGTGGAGTATTTACGGTAAACTGCCCACTTGGCAGTACAT
CAAGTGTATCATATGCCAAGTACGCCCCCTATTGACGTCAATGACGGTAAATGGCCCGCCTGGC
ATTATGCCCAGTACATGACCTTATGGGACTTTCCTACTTGGCAGTACATCTACGTATTAGTCAT
CGCTATTACCATGGTGATGCGGTTTTGGCAGTACATCAATGGGCGTGGATAGCGGTTTGACTCA
CGGGGATTTCCAAGTCTCCACCCCATTGACGTCAATGGGAGTTTGTTTTGGCACCAAAATCAAC
GGGACTTTCCAAAATGTCGTAACAACTCCGCCCCATTGACGCAAATGGGCGGTAGGCGTGTACG
GTGGGAGGTCTATATAAGCAGAGCTCGTTTAGTGAACCGTcagatcgcctggagacgcACCGGT
GCCACCATGGAAGATGCCAAAAACATTAAGAAGGGCCCAGCGCCATTCTACCCACTCGAAGACG
GGACCGCCGGCGAGCAGCTGCACAAAGCCATGAAGCGCTACGCCCTGGTGCCCGGCACCATCGC
CTTTACCGACGCACATATCGAGGTGGACATTACCTACGCCGAGTACTTCGAGATGAGCGTTCGG
CTGGCAGAAGCTATGAAGCGCTATGGGCTGAATACAAACCATCGGATCGTGGTGTGCAGCGAGA
ATAGCTTGCAGTTCTTCATGCCCGTGTTGGGTGCCCTGTTCATCGGTGTGGCTGTGGCCCCAGC
TAACGACATCTACAACGAGCGCGAGCTGCTGAACAGCATGGGCATCAGCCAGCCCACCGTCGTA
TTCGTGAGCAAGAAAGGGCTGCAAAAGATCCTCAACGTGCAAAAGAAGCTACCGATCATACAAA
AGATCATCATCATGGATAGCAAGACCGACTACCAGGGCTTCCAAAGCATGTACACCTTCGTGAC
TTCCCATTTGCCACCCGGCTTCAACGAGTACGACTTCGTGCCCGAGAGCTTCGACCGGGACAAA
ACCATCGCCCTGATCATGAACAGTAGTGGCAGTACCGGATTGCCCAAGGGCGTAGCCCTACCGC
ACCGCACCGCTTGTGTCCGATTCAGTCATGCCCGCGACCCCATCTTCGGCAACCAGATCATCCC
CGACACCGCTATCCTCAGCGTGGTGCCATTTCACCACGGCTTCGGCATGTTCACCACGCTGGGC
TACTTGATCTGCGGCTTTCGGGTCGTGCTCATGTACCGCTTCGAGGAGGAGCTATTCTTGCGCA
GCTTGCAAGACTATAAGATTCAATCTGCCCTGCTGGTGCCCACACTATTTAGCTTCTTCGCTAA
GAGCACTCTCATCGACAAGTACGACCTAAGCAACTTGCACGAGATCGCCAGCGGCGGGGCGCCG
CTCAGCAAGGAGGTAGGTGAGGCCGTGGCCAAACGCTTCCACCTACCAGGCATCCGCCAGGGCT
ACGGCCTGACAGAAACAACCAGCGCCATTCTGATCACCCCCGAAGGGGACGACAAGCCTGGCGC
AGTAGGCAAGGTGGTGCCCTTCTTCGAGGCTAAGGTGGTGGACTTGGACACAGGTAAGACACTG
GGTGTGAACCAGCGCGGCGAGCTGTGCGTCCGTGGCCCCATGATCATGAGCGGCTACGTTAACA
ACCCCGAGGCTACAAACGCTCTCATCGACAAGGACGGCTGGCTGCACAGCGGCGACATCGCCTA
CTGGGACGAGGACGAGCACTTCTTCATCGTGGACCGGCTGAAGAGCCTGATCAAATACAAGGGC
TACCAGGTAGCCCCAGCCGAACTGGAGAGCATCCTGCTGCAACACCCCAACATCTTCGACGCCG
GGGTCGCCGGCCTGCCCGACGACGATGCCGGCGAGCTGCCCGCCGCAGTCGTCGTGCTGGAACA
CGGTAAAACCATGACCGAGAAGGAGATCGTGGACTATGTGGCCAGCCAGGTTACAACCGCCAAG
AAGCTGCGCGGTGGTGTTGTGTTCGTGGACGAGGTGCCTAAAGGACTGACCGGCAAGTTGGACG
CCCGCAAGATCCGCGAGATTCTCATTAAGGCCAAGAAGGGCGGCAAGATCGCCGTGGGCTCCGG
AGAGGGCAGAGGAAGTCTGCTAACATGCGGTGACGTCGAGGAGAATCCTGGCCCAATGGAGAGC
GACGAGAGCGGCCTGCCCGCCATGGAGATCGAGTGCCGCATCACCGGCACCCTGAACGGCGTGG
AGTTCGAGCTGGTGGGCGGCGGAGAGGGCACCCCCGAGCAGGGCCGCATGACCAACAAGATGAA
GAGCACCAAAGGCGCCCTGACCTTCAGCCCCTACCTGCTGAGCCACGTGATGGGCTACGGCTTC
TACCACTTCGGCACCTACCCCAGCGGCTACGAGAACCCCTTCCTGCACGCCATCAACAACGGCG
GCTACACCAACACCCGCATCGAGAAGTACGAGGACGGCGGCGTGCTGCACGTGAGCTTCAGCTA
CCGCTACGAGGCCGGCCGCGTGATCGGCGACTTCAAGGTGATGGGCACCGGCTTCCCCGAGGAC
AGCGTGATCTTCACCGACAAGATCATCCGCAGCAACGCCACCGTGGAGCACCTGCACCCCATGG
GCGATAACGATCTGGATGGCAGCTTCACCCGCACCTTCAGCCTGCGCGACGGCGGCTACTACAG
CTCCGTGGTGGACAGCCACATGCACTTCAAGAGCGCCATCCACCCCAGCATCCTGCAGAACGGG
GGCCCCATGTTCGCCTTCCGCCGCGTGGAGGAGGATCACAGCAACACCGAGCTGGGCATCGTGG
AGTACCAGCACGCCTTCAAGACCCCGGATGCAGATGCCGGTGAAGAATAAGAGCTCGCTGATCA
GCCTCGATAAGATACATTGATGAGTTTGGACAAACCACAACTAGAATGCAGTGAAAAAAATGCT
TTATTTGTGAAATTTGTGATGCTATTGCTTTATTTGTAACCATTATAAGCTGCAATAAACAAGT
TTTAATTAAGGTCGGGCAGGAAGAGGGCCTATTTCCCATGATTCCTTCATATTTGCATATACGA
TAGAAGGCTGTTAGAGAGATAATTAGAATTAATTTGACTGTAAACACAAAGATATTAGTAGAAA
ATACGTGACGTAGAAAGTAATAATTTCTTGGGTAGTTTGCAGTTTTAAAATTATGTTTTAAAAT
GGACTATCATATGCTTACCGTAACTTGAAAGTATTTCGATTTCTTGGCTTTATATATCTTGTGG
AAAGGACGAAACACCGAAGTAGCAGAGGAGAAGAGCTCGAAAGCTCTTCTCCTCTGCTACTTTT
TTTTAAGCTTTTTTGAATTCAGCTGACGTGCCTCGGACCGCTAGGAACCCCTAGTGATGGAGTT
GGCCACTCCCTCTCTGCGCGCTCGCTCGCTCACTGAGGCCGGGCGACCAAAGGTCGCCCGACGC
CCGGGCTTTGCCCGGGCGGCCTCAGTGAGCGAGCGAGCGCGCAG
Exemplary pITR.CMV.LucT2AGFP-U6shRNA-hKCNQ4-1593 sequence
(SEQ ID NO: 263)
CTGCGCGCTCGCTCGCTCACTGAGGCCGCCCGGGCGTCGGGCGACCTTTGGTCGCCCGGCCTCA
GTGAGCGAGCGAGCGCGCAGAGAGGGAGTGGCCAACTCCATCACTAGGGGTTCCTGCGGCCGCA
CGCGTCTAGATCCCATATATGGAGTTCCGCGTTACATAACTTACGGTAAATGGCCCGCCTGGCT
GACCGCCCAACGACCCCCGCCCATTGACGTCAATAATGACGTATGTTCCCATAGTAACGCCAAT
AGGGACTTTCCATTGACGTCAATGGGTGGAGTATTTACGGTAAACTGCCCACTTGGCAGTACAT
CAAGTGTATCATATGCCAAGTACGCCCCCTATTGACGTCAATGACGGTAAATGGCCCGCCTGGC
ATTATGCCCAGTACATGACCTTATGGGACTTTCCTACTTGGCAGTACATCTACGTATTAGTCAT
CGCTATTACCATGGTGATGCGGTTTTGGCAGTACATCAATGGGCGTGGATAGCGGTTTGACTCA
CGGGGATTTCCAAGTCTCCACCCCATTGACGTCAATGGGAGTTTGTTTTGGCACCAAAATCAAC
GGGACTTTCCAAAATGTCGTAACAACTCCGCCCCATTGACGCAAATGGGCGGTAGGCGTGTACG
GTGGGAGGTCTATATAAGCAGAGCTCGTTTAGTGAACCGTcagatcgcctggagacgcACCGGT
GCCACCATGGAAGATGCCAAAAACATTAAGAAGGGCCCAGCGCCATTCTACCCACTCGAAGACG
GGACCGCCGGCGAGCAGCTGCACAAAGCCATGAAGCGCTACGCCCTGGTGCCCGGCACCATCGC
CTTTACCGACGCACATATCGAGGTGGACATTACCTACGCCGAGTACTTCGAGATGAGCGTTCGG
CTGGCAGAAGCTATGAAGCGCTATGGGCTGAATACAAACCATCGGATCGTGGTGTGCAGCGAGA
ATAGCTTGCAGTTCTTCATGCCCGTGTTGGGTGCCCTGTTCATCGGTGTGGCTGTGGCCCCAGC
TAACGACATCTACAACGAGCGCGAGCTGCTGAACAGCATGGGCATCAGCCAGCCCACCGTCGTA
TTCGTGAGCAAGAAAGGGCTGCAAAAGATCCTCAACGTGCAAAAGAAGCTACCGATCATACAAA
AGATCATCATCATGGATAGCAAGACCGACTACCAGGGCTTCCAAAGCATGTACACCTTCGTGAC
TTCCCATTTGCCACCCGGCTTCAACGAGTACGACTTCGTGCCCGAGAGCTTCGACCGGGACAAA
ACCATCGCCCTGATCATGAACAGTAGTGGCAGTACCGGATTGCCCAAGGGCGTAGCCCTACCGC
ACCGCACCGCTTGTGTCCGATTCAGTCATGCCCGCGACCCCATCTTCGGCAACCAGATCATCCC
CGACACCGCTATCCTCAGCGTGGTGCCATTTCACCACGGCTTCGGCATGTTCACCACGCTGGGC
TACTTGATCTGCGGCTTTCGGGTCGTGCTCATGTACCGCTTCGAGGAGGAGCTATTCTTGCGCA
GCTTGCAAGACTATAAGATTCAATCTGCCCTGCTGGTGCCCACACTATTTAGCTTCTTCGCTAA
GAGCACTCTCATCGACAAGTACGACCTAAGCAACTTGCACGAGATCGCCAGCGGCGGGGCGCCG
CTCAGCAAGGAGGTAGGTGAGGCCGTGGCCAAACGCTTCCACCTACCAGGCATCCGCCAGGGCT
ACGGCCTGACAGAAACAACCAGCGCCATTCTGATCACCCCCGAAGGGGACGACAAGCCTGGCGC
AGTAGGCAAGGTGGTGCCCTTCTTCGAGGCTAAGGTGGTGGACTTGGACACAGGTAAGACACTG
GGTGTGAACCAGCGCGGCGAGCTGTGCGTCCGTGGCCCCATGATCATGAGCGGCTACGTTAACA
ACCCCGAGGCTACAAACGCTCTCATCGACAAGGACGGCTGGCTGCACAGCGGCGACATCGCCTA
CTGGGACGAGGACGAGCACTTCTTCATCGTGGACCGGCTGAAGAGCCTGATCAAATACAAGGGC
TACCAGGTAGCCCCAGCCGAACTGGAGAGCATCCTGCTGCAACACCCCAACATCTTCGACGCCG
GGGTCGCCGGCCTGCCCGACGACGATGCCGGCGAGCTGCCCGCCGCAGTCGTCGTGCTGGAACA
CGGTAAAACCATGACCGAGAAGGAGATCGTGGACTATGTGGCCAGCCAGGTTACAACCGCCAAG
AAGCTGCGCGGTGGTGTTGTGTTCGTGGACGAGGTGCCTAAAGGACTGACCGGCAAGTTGGACG
CCCGCAAGATCCGCGAGATTCTCATTAAGGCCAAGAAGGGCGGCAAGATCGCCGTGGGCTCCGG
AGAGGGCAGAGGAAGTCTGCTAACATGCGGTGACGTCGAGGAGAATCCTGGCCCAATGGAGAGC
GACGAGAGCGGCCTGCCCGCCATGGAGATCGAGTGCCGCATCACCGGCACCCTGAACGGCGTGG
AGTTCGAGCTGGTGGGCGGCGGAGAGGGCACCCCCGAGCAGGGCCGCATGACCAACAAGATGAA
GAGCACCAAAGGCGCCCTGACCTTCAGCCCCTACCTGCTGAGCCACGTGATGGGCTACGGCTTC
TACCACTTCGGCACCTACCCCAGCGGCTACGAGAACCCCTTCCTGCACGCCATCAACAACGGCG
GCTACACCAACACCCGCATCGAGAAGTACGAGGACGGCGGCGTGCTGCACGTGAGCTTCAGCTA
CCGCTACGAGGCCGGCCGCGTGATCGGCGACTTCAAGGTGATGGGCACCGGCTTCCCCGAGGAC
AGCGTGATCTTCACCGACAAGATCATCCGCAGCAACGCCACCGTGGAGCACCTGCACCCCATGG
GCGATAACGATCTGGATGGCAGCTTCACCCGCACCTTCAGCCTGCGCGACGGCGGCTACTACAG
CTCCGTGGTGGACAGCCACATGCACTTCAAGAGCGCCATCCACCCCAGCATCCTGCAGAACGGG
GGCCCCATGTTCGCCTTCCGCCGCGTGGAGGAGGATCACAGCAACACCGAGCTGGGCATCGTGG
AGTACCAGCACGCCTTCAAGACCCCGGATGCAGATGCCGGTGAAGAATAAGAGCTCGCTGATCA
GCCTCGATAAGATACATTGATGAGTTTGGACAAACCACAACTAGAATGCAGTGAAAAAAATGCT
TTATTTGTGAAATTTGTGATGCTATTGCTTTATTTGTAACCATTATAAGCTGCAATAAACAAGT
TTTAATTAAGGTCGGGCAGGAAGAGGGCCTATTTCCCATGATTCCTTCATATTTGCATATACGA
TAGAAGGCTGTTAGAGAGATAATTAGAATTAATTTGACTGTAAACACAAAGATATTAGTAGAAA
ATACGTGACGTAGAAAGTAATAATTTCTTGGGTAGTTTGCAGTTTTAAAATTATGTTTTAAAAT
GGACTATCATATGCTTACCGTAACTTGAAAGTATTTCGATTTCTTGGCTTTATATATCTTGTGG
AAAGGACGAAACACCGAAGACAGTCATCCGCTCCATCCGAAGATGGAGCGGATGACTGTCTTTT
TTTTAAGCTTTTTTGAATTCAGCTGACGTGCCTCGGACCGCTAGGAACCCCTAGTGATGGAGTT
GGCCACTCCCTCTCTGCGCGCTCGCTCGCTCACTGAGGCCGGGCGACCAAAGGTCGCCCGACGC
CCGGGCTTTGCCCGGGCGGCCTCAGTGAGCGAGCGAGCGCGCA
Exemplary pITR.CMV.LucT2AGFP-U6shRNA-hKCNQ4-1677 sequence
(SEQ ID NO: 264)
CTGCGCGCTCGCTCGCTCACTGAGGCCGCCCGGGCGTCGGGCGACCTTTGGTCGCCCGGCCTCA
GTGAGCGAGCGAGCGCGCAGAGAGGGAGTGGCCAACTCCATCACTAGGGGTTCCTGCGGCCGCA
CGCGTCTAGATCCCATATATGGAGTTCCGCGTTACATAACTTACGGTAAATGGCCCGCCTGGCT
GACCGCCCAACGACCCCCGCCCATTGACGTCAATAATGACGTATGTTCCCATAGTAACGCCAAT
AGGGACTTTCCATTGACGTCAATGGGTGGAGTATTTACGGTAAACTGCCCACTTGGCAGTACAT
CAAGTGTATCATATGCCAAGTACGCCCCCTATTGACGTCAATGACGGTAAATGGCCCGCCTGGC
ATTATGCCCAGTACATGACCTTATGGGACTTTCCTACTTGGCAGTACATCTACGTATTAGTCAT
CGCTATTACCATGGTGATGCGGTTTTGGCAGTACATCAATGGGCGTGGATAGCGGTTTGACTCA
CGGGGATTTCCAAGTCTCCACCCCATTGACGTCAATGGGAGTTTGTTTTGGCACCAAAATCAAC
GGGACTTTCCAAAATGTCGTAACAACTCCGCCCCATTGACGCAAATGGGCGGTAGGCGTGTACG
GTGGGAGGTCTATATAAGCAGAGCTCGTTTAGTGAACCGTcagatcgcctggagacgcACCGGT
GCCACCATGGAAGATGCCAAAAACATTAAGAAGGGCCCAGCGCCATTCTACCCACTCGAAGACG
GGACCGCCGGCGAGCAGCTGCACAAAGCCATGAAGCGCTACGCCCTGGTGCCCGGCACCATCGC
CTTTACCGACGCACATATCGAGGTGGACATTACCTACGCCGAGTACTTCGAGATGAGCGTTCGG
CTGGCAGAAGCTATGAAGCGCTATGGGCTGAATACAAACCATCGGATCGTGGTGTGCAGCGAGA
ATAGCTTGCAGTTCTTCATGCCCGTGTTGGGTGCCCTGTTCATCGGTGTGGCTGTGGCCCCAGC
TAACGACATCTACAACGAGCGCGAGCTGCTGAACAGCATGGGCATCAGCCAGCCCACCGTCGTA
TTCGTGAGCAAGAAAGGGCTGCAAAAGATCCTCAACGTGCAAAAGAAGCTACCGATCATACAAA
AGATCATCATCATGGATAGCAAGACCGACTACCAGGGCTTCCAAAGCATGTACACCTTCGTGAC
TTCCCATTTGCCACCCGGCTTCAACGAGTACGACTTCGTGCCCGAGAGCTTCGACCGGGACAAA
ACCATCGCCCTGATCATGAACAGTAGTGGCAGTACCGGATTGCCCAAGGGCGTAGCCCTACCGC
ACCGCACCGCTTGTGTCCGATTCAGTCATGCCCGCGACCCCATCTTCGGCAACCAGATCATCCC
CGACACCGCTATCCTCAGCGTGGTGCCATTTCACCACGGCTTCGGCATGTTCACCACGCTGGGC
TACTTGATCTGCGGCTTTCGGGTCGTGCTCATGTACCGCTTCGAGGAGGAGCTATTCTTGCGCA
GCTTGCAAGACTATAAGATTCAATCTGCCCTGCTGGTGCCCACACTATTTAGCTTCTTCGCTAA
GAGCACTCTCATCGACAAGTACGACCTAAGCAACTTGCACGAGATCGCCAGCGGCGGGGCGCCG
CTCAGCAAGGAGGTAGGTGAGGCCGTGGCCAAACGCTTCCACCTACCAGGCATCCGCCAGGGCT
ACGGCCTGACAGAAACAACCAGCGCCATTCTGATCACCCCCGAAGGGGACGACAAGCCTGGCGC
AGTAGGCAAGGTGGTGCCCTTCTTCGAGGCTAAGGTGGTGGACTTGGACACAGGTAAGACACTG
GGTGTGAACCAGCGCGGCGAGCTGTGCGTCCGTGGCCCCATGATCATGAGCGGCTACGTTAACA
ACCCCGAGGCTACAAACGCTCTCATCGACAAGGACGGCTGGCTGCACAGCGGCGACATCGCCTA
CTGGGACGAGGACGAGCACTTCTTCATCGTGGACCGGCTGAAGAGCCTGATCAAATACAAGGGC
TACCAGGTAGCCCCAGCCGAACTGGAGAGCATCCTGCTGCAACACCCCAACATCTTCGACGCCG
GGGTCGCCGGCCTGCCCGACGACGATGCCGGCGAGCTGCCCGCCGCAGTCGTCGTGCTGGAACA
CGGTAAAACCATGACCGAGAAGGAGATCGTGGACTATGTGGCCAGCCAGGTTACAACCGCCAAG
AAGCTGCGCGGTGGTGTTGTGTTCGTGGACGAGGTGCCTAAAGGACTGACCGGCAAGTTGGACG
CCCGCAAGATCCGCGAGATTCTCATTAAGGCCAAGAAGGGCGGCAAGATCGCCGTGGGCTCCGG
AGAGGGCAGAGGAAGTCTGCTAACATGCGGTGACGTCGAGGAGAATCCTGGCCCAATGGAGAGC
GACGAGAGCGGCCTGCCCGCCATGGAGATCGAGTGCCGCATCACCGGCACCCTGAACGGCGTGG
AGTTCGAGCTGGTGGGCGGCGGAGAGGGCACCCCCGAGCAGGGCCGCATGACCAACAAGATGAA
GAGCACCAAAGGCGCCCTGACCTTCAGCCCCTACCTGCTGAGCCACGTGATGGGCTACGGCTTC
TACCACTTCGGCACCTACCCCAGCGGCTACGAGAACCCCTTCCTGCACGCCATCAACAACGGCG
GCTACACCAACACCCGCATCGAGAAGTACGAGGACGGCGGCGTGCTGCACGTGAGCTTCAGCTA
CCGCTACGAGGCCGGCCGCGTGATCGGCGACTTCAAGGTGATGGGCACCGGCTTCCCCGAGGAC
AGCGTGATCTTCACCGACAAGATCATCCGCAGCAACGCCACCGTGGAGCACCTGCACCCCATGG
GCGATAACGATCTGGATGGCAGCTTCACCCGCACCTTCAGCCTGCGCGACGGCGGCTACTACAG
CTCCGTGGTGGACAGCCACATGCACTTCAAGAGCGCCATCCACCCCAGCATCCTGCAGAACGGG
GGCCCCATGTTCGCCTTCCGCCGCGTGGAGGAGGATCACAGCAACACCGAGCTGGGCATCGTGG
AGTACCAGCACGCCTTCAAGACCCCGGATGCAGATGCCGGTGAAGAATAAGAGCTCGCTGATCA
GCCTCGATAAGATACATTGATGAGTTTGGACAAACCACAACTAGAATGCAGTGAAAAAAATGCT
TTATTTGTGAAATTTGTGATGCTATTGCTTTATTTGTAACCATTATAAGCTGCAATAAACAAGT
TTTAATTAAGGTCGGGCAGGAAGAGGGCCTATTTCCCATGATTCCTTCATATTTGCATATACGA
TAGAAGGCTGTTAGAGAGATAATTAGAATTAATTTGACTGTAAACACAAAGATATTAGTAGAAA
ATACGTGACGTAGAAAGTAATAATTTCTTGGGTAGTTTGCAGTTTTAAAATTATGTTTTAAAAT
GGACTATCATATGCTTACCGTAACTTGAAAGTATTTCGATTTCTTGGCTTTATATATCTTGTGG
AAAGGACGAAACACCGAAGGACGTCATTGAGCAGTACCGAAGTACTGCTCAATGACGTCCTTTT
TTTTAAGCTTTTTTGAATTCAGCTGACGTGCCTCGGACCGCTAGGAACCCCTAGTGATGGAGTT
GGCCACTCCCTCTCTGCGCGCTCGCTCGCTCACTGAGGCCGGGCGACCAAAGGTCGCCCGACGC
CCGGGCTTTGCCCGGGCGGCCTCAGTGAGCGAGCGAGCGCGCAG
Exemplary pITR.CMV.LucT2AGFP-U6shRNA-hKCNQ4-1721 sequence
(SEQ ID NO: 265)
CTGCGCGCTCGCTCGCTCACTGAGGCCGCCCGGGCGTCGGGCGACCTTTGGTCGCCCGGCCTCA
GTGAGCGAGCGAGCGCGCAGAGAGGGAGTGGCCAACTCCATCACTAGGGGTTCCTGCGGCCGCA
CGCGTCTAGATCCCATATATGGAGTTCCGCGTTACATAACTTACGGTAAATGGCCCGCCTGGCT
GACCGCCCAACGACCCCCGCCCATTGACGTCAATAATGACGTATGTTCCCATAGTAACGCCAAT
AGGGACTTTCCATTGACGTCAATGGGTGGAGTATTTACGGTAAACTGCCCACTTGGCAGTACAT
CAAGTGTATCATATGCCAAGTACGCCCCCTATTGACGTCAATGACGGTAAATGGCCCGCCTGGC
ATTATGCCCAGTACATGACCTTATGGGACTTTCCTACTTGGCAGTACATCTACGTATTAGTCAT
CGCTATTACCATGGTGATGCGGTTTTGGCAGTACATCAATGGGCGTGGATAGCGGTTTGACTCA
CGGGGATTTCCAAGTCTCCACCCCATTGACGTCAATGGGAGTTTGTTTTGGCACCAAAATCAAC
GGGACTTTCCAAAATGTCGTAACAACTCCGCCCCATTGACGCAAATGGGCGGTAGGCGTGTACG
GTGGGAGGTCTATATAAGCAGAGCTCGTTTAGTGAACCGTcagatcgcctggagacgcACCGGT
GCCACCATGGAAGATGCCAAAAACATTAAGAAGGGCCCAGCGCCATTCTACCCACTCGAAGACG
GGACCGCCGGCGAGCAGCTGCACAAAGCCATGAAGCGCTACGCCCTGGTGCCCGGCACCATCGC
CTTTACCGACGCACATATCGAGGTGGACATTACCTACGCCGAGTACTTCGAGATGAGCGTTCGG
CTGGCAGAAGCTATGAAGCGCTATGGGCTGAATACAAACCATCGGATCGTGGTGTGCAGCGAGA
ATAGCTTGCAGTTCTTCATGCCCGTGTTGGGTGCCCTGTTCATCGGTGTGGCTGTGGCCCCAGC
TAACGACATCTACAACGAGCGCGAGCTGCTGAACAGCATGGGCATCAGCCAGCCCACCGTCGTA
TTCGTGAGCAAGAAAGGGCTGCAAAAGATCCTCAACGTGCAAAAGAAGCTACCGATCATACAAA
AGATCATCATCATGGATAGCAAGACCGACTACCAGGGCTTCCAAAGCATGTACACCTTCGTGAC
TTCCCATTTGCCACCCGGCTTCAACGAGTACGACTTCGTGCCCGAGAGCTTCGACCGGGACAAA
ACCATCGCCCTGATCATGAACAGTAGTGGCAGTACCGGATTGCCCAAGGGCGTAGCCCTACCGC
ACCGCACCGCTTGTGTCCGATTCAGTCATGCCCGCGACCCCATCTTCGGCAACCAGATCATCCC
CGACACCGCTATCCTCAGCGTGGTGCCATTTCACCACGGCTTCGGCATGTTCACCACGCTGGGC
TACTTGATCTGCGGCTTTCGGGTCGTGCTCATGTACCGCTTCGAGGAGGAGCTATTCTTGCGCA
GCTTGCAAGACTATAAGATTCAATCTGCCCTGCTGGTGCCCACACTATTTAGCTTCTTCGCTAA
GAGCACTCTCATCGACAAGTACGACCTAAGCAACTTGCACGAGATCGCCAGCGGCGGGGCGCCG
CTCAGCAAGGAGGTAGGTGAGGCCGTGGCCAAACGCTTCCACCTACCAGGCATCCGCCAGGGCT
ACGGCCTGACAGAAACAACCAGCGCCATTCTGATCACCCCCGAAGGGGACGACAAGCCTGGCGC
AGTAGGCAAGGTGGTGCCCTTCTTCGAGGCTAAGGTGGTGGACTTGGACACAGGTAAGACACTG
GGTGTGAACCAGCGCGGCGAGCTGTGCGTCCGTGGCCCCATGATCATGAGCGGCTACGTTAACA
ACCCCGAGGCTACAAACGCTCTCATCGACAAGGACGGCTGGCTGCACAGCGGCGACATCGCCTA
CTGGGACGAGGACGAGCACTTCTTCATCGTGGACCGGCTGAAGAGCCTGATCAAATACAAGGGC
TACCAGGTAGCCCCAGCCGAACTGGAGAGCATCCTGCTGCAACACCCCAACATCTTCGACGCCG
GGGTCGCCGGCCTGCCCGACGACGATGCCGGCGAGCTGCCCGCCGCAGTCGTCGTGCTGGAACA
CGGTAAAACCATGACCGAGAAGGAGATCGTGGACTATGTGGCCAGCCAGGTTACAACCGCCAAG
AAGCTGCGCGGTGGTGTTGTGTTCGTGGACGAGGTGCCTAAAGGACTGACCGGCAAGTTGGACG
CCCGCAAGATCCGCGAGATTCTCATTAAGGCCAAGAAGGGCGGCAAGATCGCCGTGGGCTCCGG
AGAGGGCAGAGGAAGTCTGCTAACATGCGGTGACGTCGAGGAGAATCCTGGCCCAATGGAGAGC
GACGAGAGCGGCCTGCCCGCCATGGAGATCGAGTGCCGCATCACCGGCACCCTGAACGGCGTGG
AGTTCGAGCTGGTGGGCGGCGGAGAGGGCACCCCCGAGCAGGGCCGCATGACCAACAAGATGAA
GAGCACCAAAGGCGCCCTGACCTTCAGCCCCTACCTGCTGAGCCACGTGATGGGCTACGGCTTC
TACCACTTCGGCACCTACCCCAGCGGCTACGAGAACCCCTTCCTGCACGCCATCAACAACGGCG
GCTACACCAACACCCGCATCGAGAAGTACGAGGACGGCGGCGTGCTGCACGTGAGCTTCAGCTA
CCGCTACGAGGCCGGCCGCGTGATCGGCGACTTCAAGGTGATGGGCACCGGCTTCCCCGAGGAC
AGCGTGATCTTCACCGACAAGATCATCCGCAGCAACGCCACCGTGGAGCACCTGCACCCCATGG
GCGATAACGATCTGGATGGCAGCTTCACCCGCACCTTCAGCCTGCGCGACGGCGGCTACTACAG
CTCCGTGGTGGACAGCCACATGCACTTCAAGAGCGCCATCCACCCCAGCATCCTGCAGAACGGG
GGCCCCATGTTCGCCTTCCGCCGCGTGGAGGAGGATCACAGCAACACCGAGCTGGGCATCGTGG
AGTACCAGCACGCCTTCAAGACCCCGGATGCAGATGCCGGTGAAGAATAAGAGCTCGCTGATCA
GCCTCGATAAGATACATTGATGAGTTTGGACAAACCACAACTAGAATGCAGTGAAAAAAATGCT
TTATTTGTGAAATTTGTGATGCTATTGCTTTATTTGTAACCATTATAAGCTGCAATAAACAAGT
TTTAATTAAGGTCGGGCAGGAAGAGGGCCTATTTCCCATGATTCCTTCATATTTGCATATACGA
TAGAAGGCTGTTAGAGAGATAATTAGAATTAATTTGACTGTAAACACAAAGATATTAGTAGAAA
ATACGTGACGTAGAAAGTAATAATTTCTTGGGTAGTTTGCAGTTTTAAAATTATGTTTTAAAAT
GGACTATCATATGCTTACCGTAACTTGAAAGTATTTCGATTTCTTGGCTTTATATATCTTGTGG
AAAGGACGAAACACCGGCCGGATCAAGAGCCTGCAAACGAATTTGCAGGCTCTTGATCCGGCTT
TTTTAAGCTTTTTTGAATTCAGCTGACGTGCCTCGGACCGCTAGGAACCCCTAGTGATGGAGTT
GGCCACTCCCTCTCTGCGCGCTCGCTCGCTCACTGAGGCCGGGCGACCAAAGGTCGCCCGACGC
CCGGGCTTTGCCCGGGCGGCCTCAGTGAGCGAGCGAGCGCGCAG
Exemplary pITR.CMV.LucT2AGFP-U6shRNA-hKCNQ4-1827 sequence
(SEQ ID NO: 266)
CTGCGCGCTCGCTCGCTCACTGAGGCCGCCCGGGCGTCGGGCGACCTTTGGTCGCCCGGCCTCA
GTGAGCGAGCGAGCGCGCAGAGAGGGAGTGGCCAACTCCATCACTAGGGGTTCCTGCGGCCGCA
CGCGTCTAGATCCCATATATGGAGTTCCGCGTTACATAACTTACGGTAAATGGCCCGCCTGGCT
GACCGCCCAACGACCCCCGCCCATTGACGTCAATAATGACGTATGTTCCCATAGTAACGCCAAT
AGGGACTTTCCATTGACGTCAATGGGTGGAGTATTTACGGTAAACTGCCCACTTGGCAGTACAT
CAAGTGTATCATATGCCAAGTACGCCCCCTATTGACGTCAATGACGGTAAATGGCCCGCCTGGC
ATTATGCCCAGTACATGACCTTATGGGACTTTCCTACTTGGCAGTACATCTACGTATTAGTCAT
CGCTATTACCATGGTGATGCGGTTTTGGCAGTACATCAATGGGCGTGGATAGCGGTTTGACTCA
CGGGGATTTCCAAGTCTCCACCCCATTGACGTCAATGGGAGTTTGTTTTGGCACCAAAATCAAC
GGGACTTTCCAAAATGTCGTAACAACTCCGCCCCATTGACGCAAATGGGCGGTAGGCGTGTACG
GTGGGAGGTCTATATAAGCAGAGCTCGTTTAGTGAACCGTcagatcgcctggagacgcACCGGT
GCCACCATGGAAGATGCCAAAAACATTAAGAAGGGCCCAGCGCCATTCTACCCACTCGAAGACG
GGACCGCCGGCGAGCAGCTGCACAAAGCCATGAAGCGCTACGCCCTGGTGCCCGGCACCATCGC
CTTTACCGACGCACATATCGAGGTGGACATTACCTACGCCGAGTACTTCGAGATGAGCGTTCGG
CTGGCAGAAGCTATGAAGCGCTATGGGCTGAATACAAACCATCGGATCGTGGTGTGCAGCGAGA
ATAGCTTGCAGTTCTTCATGCCCGTGTTGGGTGCCCTGTTCATCGGTGTGGCTGTGGCCCCAGC
TAACGACATCTACAACGAGCGCGAGCTGCTGAACAGCATGGGCATCAGCCAGCCCACCGTCGTA
TTCGTGAGCAAGAAAGGGCTGCAAAAGATCCTCAACGTGCAAAAGAAGCTACCGATCATACAAA
AGATCATCATCATGGATAGCAAGACCGACTACCAGGGCTTCCAAAGCATGTACACCTTCGTGAC
TTCCCATTTGCCACCCGGCTTCAACGAGTACGACTTCGTGCCCGAGAGCTTCGACCGGGACAAA
ACCATCGCCCTGATCATGAACAGTAGTGGCAGTACCGGATTGCCCAAGGGCGTAGCCCTACCGC
ACCGCACCGCTTGTGTCCGATTCAGTCATGCCCGCGACCCCATCTTCGGCAACCAGATCATCCC
CGACACCGCTATCCTCAGCGTGGTGCCATTTCACCACGGCTTCGGCATGTTCACCACGCTGGGC
TACTTGATCTGCGGCTTTCGGGTCGTGCTCATGTACCGCTTCGAGGAGGAGCTATTCTTGCGCA
GCTTGCAAGACTATAAGATTCAATCTGCCCTGCTGGTGCCCACACTATTTAGCTTCTTCGCTAA
GAGCACTCTCATCGACAAGTACGACCTAAGCAACTTGCACGAGATCGCCAGCGGCGGGGCGCCG
CTCAGCAAGGAGGTAGGTGAGGCCGTGGCCAAACGCTTCCACCTACCAGGCATCCGCCAGGGCT
ACGGCCTGACAGAAACAACCAGCGCCATTCTGATCACCCCCGAAGGGGACGACAAGCCTGGCGC
AGTAGGCAAGGTGGTGCCCTTCTTCGAGGCTAAGGTGGTGGACTTGGACACAGGTAAGACACTG
GGTGTGAACCAGCGCGGCGAGCTGTGCGTCCGTGGCCCCATGATCATGAGCGGCTACGTTAACA
ACCCCGAGGCTACAAACGCTCTCATCGACAAGGACGGCTGGCTGCACAGCGGCGACATCGCCTA
CTGGGACGAGGACGAGCACTTCTTCATCGTGGACCGGCTGAAGAGCCTGATCAAATACAAGGGC
TACCAGGTAGCCCCAGCCGAACTGGAGAGCATCCTGCTGCAACACCCCAACATCTTCGACGCCG
GGGTCGCCGGCCTGCCCGACGACGATGCCGGCGAGCTGCCCGCCGCAGTCGTCGTGCTGGAACA
CGGTAAAACCATGACCGAGAAGGAGATCGTGGACTATGTGGCCAGCCAGGTTACAACCGCCAAG
AAGCTGCGCGGTGGTGTTGTGTTCGTGGACGAGGTGCCTAAAGGACTGACCGGCAAGTTGGACG
CCCGCAAGATCCGCGAGATTCTCATTAAGGCCAAGAAGGGCGGCAAGATCGCCGTGGGCTCCGG
AGAGGGCAGAGGAAGTCTGCTAACATGCGGTGACGTCGAGGAGAATCCTGGCCCAATGGAGAGC
GACGAGAGCGGCCTGCCCGCCATGGAGATCGAGTGCCGCATCACCGGCACCCTGAACGGCGTGG
AGTTCGAGCTGGTGGGCGGCGGAGAGGGCACCCCCGAGCAGGGCCGCATGACCAACAAGATGAA
GAGCACCAAAGGCGCCCTGACCTTCAGCCCCTACCTGCTGAGCCACGTGATGGGCTACGGCTTC
TACCACTTCGGCACCTACCCCAGCGGCTACGAGAACCCCTTCCTGCACGCCATCAACAACGGCG
GCTACACCAACACCCGCATCGAGAAGTACGAGGACGGCGGCGTGCTGCACGTGAGCTTCAGCTA
CCGCTACGAGGCCGGCCGCGTGATCGGCGACTTCAAGGTGATGGGCACCGGCTTCCCCGAGGAC
AGCGTGATCTTCACCGACAAGATCATCCGCAGCAACGCCACCGTGGAGCACCTGCACCCCATGG
GCGATAACGATCTGGATGGCAGCTTCACCCGCACCTTCAGCCTGCGCGACGGCGGCTACTACAG
CTCCGTGGTGGACAGCCACATGCACTTCAAGAGCGCCATCCACCCCAGCATCCTGCAGAACGGG
GGCCCCATGTTCGCCTTCCGCCGCGTGGAGGAGGATCACAGCAACACCGAGCTGGGCATCGTGG
AGTACCAGCACGCCTTCAAGACCCCGGATGCAGATGCCGGTGAAGAATAAGAGCTCGCTGATCA
GCCTCGATAAGATACATTGATGAGTTTGGACAAACCACAACTAGAATGCAGTGAAAAAAATGCT
TTATTTGTGAAATTTGTGATGCTATTGCTTTATTTGTAACCATTATAAGCTGCAATAAACAAGT
TTTAATTAAGGTCGGGCAGGAAGAGGGCCTATTTCCCATGATTCCTTCATATTTGCATATACGA
TAGAAGGCTGTTAGAGAGATAATTAGAATTAATTTGACTGTAAACACAAAGATATTAGTAGAAA
ATACGTGACGTAGAAAGTAATAATTTCTTGGGTAGTTTGCAGTTTTAAAATTATGTTTTAAAAT
GGACTATCATATGCTTACCGTAACTTGAAAGTATTTCGATTTCTTGGCTTTATATATCTTGTGG
AAAGGACGAAACACCGGGTGGATGAAATCAGCATGATCGAAATCATGCTGATTTCATCCACCTT
TTTTAAGCTTTTTTGAATTCAGCTGACGTGCCTCGGACCGCTAGGAACCCCTAGTGATGGAGTT
GGCCACTCCCTCTCTGCGCGCTCGCTCGCTCACTGAGGCCGGGCGACCAAAGGTCGCCCGACGC
CCGGGCTTTGCCCGGGCGGCCTCAGTGAGCGAGCGAGCGCGCAG

Exemplary hKCNQ4 codon optimized to resist CRISPR, some with sgRNA Construct Sequences

Exemplary pITR.CMV.hKCNQ4codop.mScarlet
sequence
(SEQ ID NO: 267)
CTGCGCGCTCGCTCGCTCACTGAGGCCGCCCGGGCGTCGGGCGACCT
TTGGTCGCCCGGCCTCAGTGAGCGAGCGAGCGCGCAGAGAGGGAGTG
GCCAACTCCATCACTAGGGGTTCCTGCGGCCGCACGCGTCTAGATCC
CATATATGGAGTTCCGCGTTACATAACTTACGGTAAATGGCCCGCCT
GGCTGACCGCCCAACGACCCCCGCCCATTGACGTCAATAATGACGTA
TGTTCCCATAGTAACGCCAATAGGGACTTTCCATTGACGTCAATGGG
TGGAGTATTTACGGTAAACTGCCCACTTGGCAGTACATCAAGTGTAT
CATATGCCAAGTACGCCCCCTATTGACGTCAATGACGGTAAATGGCC
CGCCTGGCATTATGCCCAGTACATGACCTTATGGGACTTTCCTACTT
GGCAGTACATCTACGTATTAGTCATCGCTATTACCATGGTGATGCGG
TTTTGGCAGTACATCAATGGGCGTGGATAGCGGTTTGACTCACGGGG
ATTTCCAAGTCTCCACCCCATTGACGTCAATGGGAGTTTGTTTTGGC
ACCAAAATCAACGGGACTTTCCAAAATGTCGTAACAACTCCGCCCCA
TTGACGCAAATGGGCGGTAGGCGTGTACGGTGGGAGGTCTATATAAG
CAGAGCTCGTTTAGTGAACCGTCAGATCGCCTGGAGACGCACCGGTG
CCACCATGGCTGAAGCCCCTCCTAGAAGGCTTGGACTGGGACCTCCT
CCTGGGGATGCTCCTAGAGCTGAACTGGTGGCTCTGACAGCCGTGCA
GTCTGAACAAGGCGAAGCTGGTGGCGGCGGATCTCCACGTAGACTTG
GACTGCTGGGAAGCCCTCTTCCTCCTGGTGCTCCACTTCCTGGACCT
GGCAGTGGATCTGGATCTGCCTGTGGCCAGAGAAGCTCTGCCGCTCA
CAAGAGATACCGGCGGCTGCAGAACTGGGTGTACAACGTGCTGGAAA
GACCCAGAGGCTGGGCCTTCGTGTACCACGTGTTCATCTTTCTGCTG
GTGTTCAGCTGCCTGGTGCTGTCCGTGCTGAGCACCATCCAAGAACA
TCAAGAGCTGGCTAACGAGTGCCTGTTAATACTGGAGTTTGTGATGA
TTGTGGTGTTCGGCCTCGAGTACATCGTCCGCGTTTGGAGCGCCGGC
TGCTGCTGCAGATATAGAGGTTGGCAAGGCAGATTCCGCTTCGCCAG
AAAGCCCTTCTGCGTGATCGACTTCATCGTGTTCGTGGCCAGCGTGG
CCGTGATTGCTGCTGGCACACAGGGCAACATCTTCGCCACAAGCGCC
CTGCGGAGCATGCGGTTTCTGCAGATCCTGAGAATGGTCCGAATGGA
CAGAAGAGGCGGCACCTGGAAGCTGCTGGGCTCTGTGGTGTACGCCC
ACAGCAAAGAGCTGATCACCGCCTGGTACATCGGATTTCTGGTGCTG
ATCTTCGCCTCCTTCCTGGTGTACCTGGCCGAGAAGGACGCCAACAG
CGACTTTAGCAGCTACGCCGACTCTCTTTGGTGGGGCACCATCACAC
TGACCACCATCGGCTACGGCGACAAGACCCCTCACACATGGCTGGGA
AGAGTGCTGGCCGCTGGATTTGCTCTGCTGGGCATCAGCTTTTTCGC
CCTGCCTGCCGGAATCCTCGGATCTGGCTTTGCCCTGAAGGTGCAAG
AGCAGCACCGGCAGAAGCACTTCGAGAAGAGAAGAATGCCTGCCGCC
AACCTGATTCAGGCCGCTTGGAGACTGTACAGCACCGACATGAGCAG
AGCCTACCTGACCGCCACGTGGTATTATTACGACTCGATCCTGCCTA
GCTTCCGCGAACTGGCCCTGCTGTTTGAGCATGTGCAGAGAGCCAGA
AACGGCGGCCTCAGACCTCTGGAAGTTCGGAGAGCACCTGTGCCTGA
TGGCGCCCCTTCTAGATATCCTCCAGTGGCCACCTGTCACAGACCCG
GCAGCACATCTTTTTGCCCTGGCGAGTCTAGCCGGATGGGCATCAAG
GACAGAATCAGAATGGGCAGCAGCCAGCGGAGAACAGGCCCTTCTAA
ACAGCATCTGGCCCCTCCAACCATGCCTACAAGCCCTAGCTCTGAGC
AAGTGGGCGAAGCCACCTCTCCTACCAAGGTGCAGAAGTCCTGGTCC
TTCAACGACCGGACCAGATTCAGAGCCAGCCTGAGACTGAAGCCCAG
AACCTCTGCCGAGGATGCCCCTTCTGAAGAGGTGGCCGAAGAGAAGT
CCTACCAGTGCGAGCTGACCGTGGACGACATCATGCCAGCCGTGAAA
ACCGTGATACGGTCTATCCGGATCCTGAAGTTCCTGGTGGCCAAGCG
GAAGTTCAAAGAGACACTGCGGCCCTACGACGTGAAGGACGTGATCG
AGCAGTATTCTGCCGGCCACCTGGACATGCTGGGCAGAATCAAGAGC
CTGCAGACCAGAGTGGACCAGATCGTTGGAAGAGGCCCAGGCGACAG
AAAGGCCAGAGAGAAGGGCGATAAGGGCCCATCTGATGCCGAGGTTG
TCGACGAGATATCAATGATGGGCAGAGTGGTCAAGGTGGAAAAACAG
GTGCAGAGCATCGAGCACAAGCTGGACCTGCTGCTGGGATTCTACAG
CCGGTGTCTGAGAAGCGGCACATCTGCATCTCTGGGCGCTGTGCAGG
TCCCACTGTTCGACCCTGATATCACCAGCGACTATCACAGCCCCGTG
GACCACGAGGACATCTCCGTTTCTGCTCAGACCCTGAGCATCAGCAG
ATCCGTGTCCACCAACATGGACGGATCCCGGGCTGACTACAAAGACC
ATGACGGTGATTATAAAGATCATGACATCGACTACAAGGATGACGAT
GACAAGGGCTCCGGAGAGGGCAGAGGAAGTCTGCTAACATGCGGTGA
CGTCGAGGAGAATCCTGGCCCAATGGTGAGCAAGGGCGAGGCAGTGA
TCAAGGAGTTCATGCGGTTCAAGGTGCACATGGAGGGCTCCATGAAC
GGCCACGAGTTCGAGATCGAGGGCGAGGGCGAGGGCCGCCCCTACGA
GGGCACCCAGACCGCCAAGCTGAAGGTGACCAAGGGTGGCCCCCTGC
CCTTCTCCTGGGACATCCTGTCCCCTCAGTTCATGTACGGCTCCAGG
GCCTTCATCAAGCACCCCGCCGACATCCCCGACTACTATAAGCAGTC
CTTCCCCGAGGGCTTCAAGTGGGAGCGCGTGATGAACTTCGAGGACG
GCGGCGCCGTGACCGTGACCCAGGACACCTCCCTGGAGGACGGCACC
CTGATCTACAAGGTGAAGCTCCGCGGCACCAACTTCCCTCCTGACGG
CCCCGTAATGCAGAAGAAGACAATGGGCTGGGAAGCGTCCACCGAGC
GGTTGTACCCCGAGGACGGCGTGCTGAAGGGCGACATTAAGATGGCC
CTGCGCCTGAAGGACGGCGGCCGCTACCTGGCGGACTTCAAGACCAC
CTACAAGGCCAAGAAGCCCGTGCAGATGCCCGGCGCCTACAACGTCG
ACCGCAAGTTGGACATCACCTCCCACAACGAGGACTACACCGTGGTG
GAACAGTACGAACGCTCCGAGGGCCGCCACTCCACCGGCGGCATGGA
CGAGCTGTACAAGTAATAAGAGCTCGCTGATCAGCCTCGACTGTGCC
TTCTAGTTGCCAGCCATCTGTTGTTTGCCCCTCCCCCGTGCCTTCCT
TGACCCTGGAAGGTGCCACTCCCACTGTCCTTTCCTAATAAAATGAG
GAAATTGCATCGCATTGTCTGAGTAGGTGTCATTCTATTCTGGGGGG
TGGGGTGGGGCAGGACAGCAAGGGGGAGGATTGGGAAGACAATAGCA
GGCATGCTGGGGATGCGGTGGGCTCTATGGAAGCTTGAATTCAGCTG
ACGTGCCTCGGACCGCTAGGAACCCCTAGTGATGGAGTTGGCCACTC
CCTCTCTGCGCGCTCGCTCGCTCACTGAGGCCGGGCGACCAAAGGTC
GCCCGACGCCCGGGCTTTGCCCGGGCGGCCTCAGTGAGCGAGCGAGC
GCGCAG
Exemplary pITR.CMV.hKCNQ4codop_v2.mScarlet
sequence
(SEQ ID NO: 268)
CTGCGCGCTCGCTCGCTCACTGAGGCCGCCCGGGCGTCGGGCGACCT
TTGGTCGCCCGGCCTCAGTGAGCGAGCGAGCGCGCAGAGAGGGAGTG
GCCAACTCCATCACTAGGGGTTCCTGCGGCCGCACGCGTCTAGATCC
CATATATGGAGTTCCGCGTTACATAACTTACGGTAAATGGCCCGCCT
GGCTGACCGCCCAACGACCCCCGCCCATTGACGTCAATAATGACGTA
TGTTCCCATAGTAACGCCAATAGGGACTTTCCATTGACGTCAATGGG
TGGAGTATTTACGGTAAACTGCCCACTTGGCAGTACATCAAGTGTAT
CATATGCCAAGTACGCCCCCTATTGACGTCAATGACGGTAAATGGCC
CGCCTGGCATTATGCCCAGTACATGACCTTATGGGACTTTCCTACTT
GGCAGTACATCTACGTATTAGTCATCGCTATTACCATGGTGATGCGG
TTTTGGCAGTACATCAATGGGCGTGGATAGCGGTTTGACTCACGGGG
ATTTCCAAGTCTCCACCCCATTGACGTCAATGGGAGTTTGTTTTGGC
ACCAAAATCAACGGGACTTTCCAAAATGTCGTAACAACTCCGCCCCA
TTGACGCAAATGGGCGGTAGGCGTGTACGGTGGGAGGTCTATATAAG
CAGAGCTCGTTTAGTGAACCGTcagatcgcctggagacgcACCGGTG
CCACCATGGCTGAAGCCCCTCCTAGAAGGCTTGGACTGGGACCTCCT
CCTGGGGATGCTCCTAGAGCTGAACTGGTGGCTCTGACAGCCGTGCA
GTCTGAACAAGGCGAAGCTGGTGGCGGCGGATCTCCACGTAGACTTG
GACTGCTGGGAAGCCCTCTTCCTCCTGGTGCTCCACTTCCTGGACCT
GGCAGTGGATCTGGATCTGCCTGTGGCCAGAGAAGCTCTGCCGCTCA
CAAGAGATACCGGCGGCTGCAGAACTGGGTGTACAACGTGCTGGAAA
GACCCAGAGGCTGGGCCTTCGTGTACCACGTGTTCATCTTTCTGCTG
GTGTTCAGCTGCCTGGTGCTGTCCGTGCTGAGCACCATCCAAGAACA
TCAAGAGCTGGCTAACGAGTGCCTGTTAATACTGGAGTTTGTGATGA
TTGTGGTGTTCGGCCTCGAGTACATCGTCCGCGTTTGGAGCGCCGGC
TGCTGCTGCAGATATAGAGGTTGGCAAGGCAGATTCCGCTTCGCCAG
AAAGCCCTTCTGCGTGATCGACTTCATCGTGTTCGTGGCCAGCGTGG
CCGTGATTGCTGCTGGCACACAGGGCAACATCTTCGCCACAAGCGCC
CTGCGGAGCATGCGGTTTCTGCAGATCCTGAGAATGGTCCGAATGGA
CAGAAGAGGCGGCACCTGGAAGCTGCTGGGCTCTGTGGTGTACGCCC
ACAGCAAAGAGCTGATCACCGCCTGGTACATCGGATTTCTGGTGCTG
ATCTTCGCCTCCTTCCTGGTGTACCTGGCCGAGAAGGACGCCAACAG
CGACTTTAGCAGCTACGCCGACTCTCTTTGGTGGGGCACCATCACAC
TGACCACCATCGGCTACGGCGACAAGACCCCTCACACATGGCTGGGA
AGAGTGCTGGCCGCTGGATTTGCTCTGCTGGGCATCAGCTTTTTCGC
CCTGCCTGCCGGAATCCTCGGATCTGGCTTTGCCCTGAAGGTGCAAG
AGCAGCATAGACAAAAGCATTTTGAAAAGAGAAGAATGCCTGCCGCC
AACCTGATTCAGGCCGCTTGGAGACTGTACAGCACCGACATGAGCAG
AGCCTACCTGACCGCCACGTGGTATTATTACGATTCGATCCTGCCTA
GCTTCCGCGAACTGGCCCTGCTGTTTGAGCATGTGCAGAGAGCCAGA
AACGGCGGCCTCAGACCTCTGGAAGTTCGGAGAGCACCTGTGCCTGA
TGGCGCCCCTTCTAGATATCCTCCAGTGGCCACCTGTCACAGACCCG
GCAGCACATCTTTTTGCCCTGGCGAGTCTAGCCGGATGGGCATCAAG
GACAGAATCAGAATGGGCAGCAGCCAGCGGAGAACAGGCCCTTCTAA
ACAGCATCTGGCCCCTCCAACCATGCCTACAAGCCCTAGCTCTGAGC
AAGTGGGCGAAGCCACCTCTCCTACCAAGGTGCAGAAGTCCTGGTCC
TTCAACGACCGGACCAGATTTAGAGCCAGCCTCCGGCTGAAGCCCAG
AACCTCTGCCGAGGATGCCCCTTCTGAAGAGGTGGCCGAAGAGAAGT
CCTACCAGTGCGAGCTGACCGTGGACGACATCATGCCAGCCGTGAAA
ACCGTGATAAGGTCTATACGCATCCTGAAGTTCCTGGTGGCCAAGCG
GAAGTTCAAAGAGACACTGCGGCCCTACGACGTGAAGGACGTGATCG
AGCAGTATTCTGCCGGCCACCTGGACATGCTGGGCAGAATCAAGAGC
CTGCAGACCAGAGTGGACCAGATCGTTGGAAGAGGCCCAGGCGACAG
AAAGGCCAGAGAGAAGGGCGATAAGGGCCCATCTGATGCCGAGGTTG
TCGACGAGATATCAATGATGGGCAGAGTCGTGAAAGTCGAAAAACAG
GTGCAGAGCATCGAGCACAAGCTGGACCTGCTGCTGGGATTCTACAG
CCGGTGTCTGAGAAGCGGCACATCTGCATCTCTGGGCGCTGTGCAGG
TCCCACTGTTCGATCCTGATATTACGAGCGATTATCACAGCCCCGTG
GACCACGAGGACATCTCCGTTTCTGCTCAGACCCTGAGCATCAGCAG
ATCCGTGTCCACCAACATGGACGGATCCCGGGCTGACTACAAAGACC
ATGACGGTGATTATAAAGATCATGACATCGACTACAAGGATGACGAT
GACAAGGGCTCCGGAGAGGGCAGAGGAAGTCTGCTAACATGCGGTGA
CGTCGAGGAGAATCCTGGCCCAATGGTGAGCAAGGGCGAGGCAGTGA
TCAAGGAGTTCATGCGGTTCAAGGTGCACATGGAGGGCTCCATGAAC
GGCCACGAGTTCGAGATCGAGGGCGAGGGCGAGGGCCGCCCCTACGA
GGGCACCCAGACCGCCAAGCTGAAGGTGACCAAGGGTGGCCCCCTGC
CCTTCTCCTGGGACATCCTGTCCCCTCAGTTCATGTACGGCTCCAGG
GCCTTCATCAAGCACCCCGCCGACATCCCCGACTACTATAAGCAGTC
CTTCCCCGAGGGCTTCAAGTGGGAGCGCGTGATGAACTTCGAGGACG
GCGGCGCCGTGACCGTGACCCAGGACACCTCCCTGGAGGACGGCACC
CTGATCTACAAGGTGAAGCTCCGCGGCACCAACTTCCCTCCTGACGG
CCCCGTAATGCAGAAGAAGACAATGGGCTGGGAAGCGTCCACCGAGC
GGTTGTACCCCGAGGACGGCGTGCTGAAGGGCGACATTAAGATGGCC
CTGCGCCTGAAGGACGGCGGCCGCTACCTGGCGGACTTCAAGACCAC
CTACAAGGCCAAGAAGCCCGTGCAGATGCCCGGCGCCTACAACGTCG
ACCGCAAGTTGGACATCACCTCCCACAACGAGGACTACACCGTGGTG
GAACAGTACGAACGCTCCGAGGGCCGCCACTCCACCGGCGGCATGGA
CGAGCTGTACAAGTAATAAGAGCTCGCTGATCAGCCTCGACTGTGCC
TTCTAGTTGCCAGCCATCTGTTGTTTGCCCCTCCCCCGTGCCTTCCT
TGACCCTGGAAGGTGCCACTCCCACTGTCCTTTCCTAATAAAATGAG
GAAATTGCATCGCATTGTCTGAGTAGGTGTCATTCTATTCTGGGGGG
TGGGGTGGGGCAGGACAGCAAGGGGGAGGATTGGGAAGACAATAGCA
GGCATGCTGGGGATGCGGTGGGCTCTATGGAAGCTTGAATTCAGCTG
ACGTGCCTCGGACCGCTAGGAACCCCTAGTGATGGAGTTGGCCACTC
CCTCTCTGCGCGCTCGCTCGCTCACTGAGGCCGGGCGACCAAAGGTC
GCCCGACGCCCGGGCTTTGCCCGGGCGGCCTCAGTGAGCGAGCGAGC
GCGCAG
Exemplary pITR-CMV.hKCNQ4codop.U6-hsammu386Fw
sequence
(SEQ ID NO: 269)
CTGCGCGCTCGCTCGCTCACTGAGGCCGCCCGGGCGTCGGGCGACCT
TTGGTCGCCCGGCCTCAGTGAGCGAGCGAGCGCGCAGAGAGGGAGTG
GCCAACTCCATCACTAGGGGTTCCTGCGGCCGCACGCGTCTAGATCC
CATATATGGAGTTCCGCGTTACATAACTTACGGTAAATGGCCCGCCT
GGCTGACCGCCCAACGACCCCCGCCCATTGACGTCAATAATGACGTA
TGTTCCCATAGTAACGCCAATAGGGACTTTCCATTGACGTCAATGGG
TGGAGTATTTACGGTAAACTGCCCACTTGGCAGTACATCAAGTGTAT
CATATGCCAAGTACGCCCCCTATTGACGTCAATGACGGTAAATGGCC
CGCCTGGCATTATGCCCAGTACATGACCTTATGGGACTTTCCTACTT
GGCAGTACATCTACGTATTAGTCATCGCTATTACCATGGTGATGCGG
TTTTGGCAGTACATCAATGGGCGTGGATAGCGGTTTGACTCACGGGG
ATTTCCAAGTCTCCACCCCATTGACGTCAATGGGAGTTTGTTTTGGC
ACCAAAATCAACGGGACTTTCCAAAATGTCGTAACAACTCCGCCCCA
TTGACGCAAATGGGCGGTAGGCGTGTACGGTGGGAGGTCTATATAAG
CAGAGCTCGTTTAGTGAACCGTCGTTTAGTGAACCGTcagatcgcct
ggagacgcACCGGTGCCACCATGGCTGAAGCCCCTCCTAGAAGGCTT
GGACTGGGACCTCCTCCTGGGGATGCTCCTAGAGCTGAACTGGTGGC
TCTGACAGCCGTGCAGTCTGAACAAGGCGAAGCTGGTGGCGGCGGAT
CTCCACGTAGACTTGGACTGCTGGGAAGCCCTCTTCCTCCTGGTGCT
CCACTTCCTGGACCTGGCAGTGGATCTGGATCTGCCTGTGGCCAGAG
AAGCTCTGCCGCTCACAAGAGATACCGGCGGCTGCAGAACTGGGTGT
ACAACGTGCTGGAAAGACCCAGAGGCTGGGCCTTCGTGTACCACGTG
TTCATCTTTCTGCTGGTGTTCAGCTGCCTGGTGCTGTCCGTGCTGAG
CACCATCCAAGAACATCAAGAGCTGGCTAACGAGTGCCTGTTAATAC
TGGAGTTTGTGATGATTGTGGTGTTCGGCCTCGAGTACATCGTCCGC
GTTTGGAGCGCCGGCTGCTGCTGCAGATATAGAGGTTGGCAAGGCAG
ATTCCGCTTCGCCAGAAAGCCCTTCTGCGTGATCGACTTCATCGTGT
TCGTGGCCAGCGTGGCCGTGATTGCTGCTGGCACACAGGGCAACATC
TTCGCCACAAGCGCCCTGCGGAGCATGCGGTTTCTGCAGATCCTGAG
AATGGTCCGAATGGACAGAAGAGGCGGCACCTGGAAGCTGCTGGGCT
CTGTGGTGTACGCCCACAGCAAAGAGCTGATCACCGCCTGGTACATC
GGATTTCTGGTGCTGATCTTCGCCTCCTTCCTGGTGTACCTGGCCGA
GAAGGACGCCAACAGCGACTTTAGCAGCTACGCCGACTCTCTTTGGT
GGGGCACCATCACACTGACCACCATCGGCTACGGCGACAAGACCCCT
CACACATGGCTGGGAAGAGTGCTGGCCGCTGGATTTGCTCTGCTGGG
CATCAGCTTTTTCGCCCTGCCTGCCGGAATCCTCGGATCTGGCTTTG
CCCTGAAGGTGCAAGAGCAGCACCGGCAGAAGCACTTCGAGAAGAGA
AGAATGCCTGCCGCCAACCTGATTCAGGCCGCTTGGAGACTGTACAG
CACCGACATGAGCAGAGCCTACCTGACCGCCACGTGGTATTATTACG
ACTCGATCCTGCCTAGCTTCCGCGAACTGGCCCTGCTGTTTGAGCAT
GTGCAGAGAGCCAGAAACGGCGGCCTCAGACCTCTGGAAGTTCGGAG
AGCACCTGTGCCTGATGGCGCCCCTTCTAGATATCCTCCAGTGGCCA
CCTGTCACAGACCCGGCAGCACATCTTTTTGCCCTGGCGAGTCTAGC
CGGATGGGCATCAAGGACAGAATCAGAATGGGCAGCAGCCAGCGGAG
AACAGGCCCTTCTAAACAGCATCTGGCCCCTCCAACCATGCCTACAA
GCCCTAGCTCTGAGCAAGTGGGCGAAGCCACCTCTCCTACCAAGGTG
CAGAAGTCCTGGTCCTTCAACGACCGGACCAGATTCAGAGCCAGCCT
GAGACTGAAGCCCAGAACCTCTGCCGAGGATGCCCCTTCTGAAGAGG
TGGCCGAAGAGAAGTCCTACCAGTGCGAGCTGACCGTGGACGACATC
ATGCCAGCCGTGAAAACCGTGATACGGTCTATCCGGATCCTGAAGTT
CCTGGTGGCCAAGCGGAAGTTCAAAGAGACACTGCGGCCCTACGACG
TGAAGGACGTGATCGAGCAGTATTCTGCCGGCCACCTGGACATGCTG
GGCAGAATCAAGAGCCTGCAGACCAGAGTGGACCAGATCGTTGGAAG
AGGCCCAGGCGACAGAAAGGCCAGAGAGAAGGGCGATAAGGGCCCAT
CTGATGCCGAGGTTGTCGACGAGATATCAATGATGGGCAGAGTGGTC
AAGGTGGAAAAACAGGTGCAGAGCATCGAGCACAAGCTGGACCTGCT
GCTGGGATTCTACAGCCGGTGTCTGAGAAGCGGCACATCTGCATCTC
TGGGCGCTGTGCAGGTCCCACTGTTCGACCCTGATATCACCAGCGAC
TATCACAGCCCCGTGGACCACGAGGACATCTCCGTTTCTGCTCAGAC
CCTGAGCATCAGCAGATCCGTGTCCACCAACATGGACTAATAAGAGC
TCGCTGATCAGCCTCGACTGTGCCTTCTAGTTGCCAGCCATCTGTTG
TTTGCCCCTCCCCCGTGCCTTCCTTGACCCTGGAAGGTGCCACTCCC
ACTGTCCTTTCCTAATAAAATGAGGAAATTGCATCGCATTGTCTGAG
TAGGTGTCATTCTATTCTGGGGGGTGGGGTGGGGCAGGACAGCAAGG
GGGAGGATTGGGAAGACAATAGCAGGCATGCTGGGGATGCGGTGGGC
TCTATGGTTAATTAAGGTCGGGCAGGAAGAGGGCCTATTTCCCATGA
TTCCTTCATATTTGCATATACGATACAAGGCTGTTAGAGAGATAATT
AGAATTAATTTGACTGTAAACACAAAGATATTAGTACAAAATACGTG
ACGTAGAAAGTAATAATTTCTTGGGTAGTTTGCAGTTTTAAAATTAT
GTTTTAAAATGGACTATCATATGCTTACCGTAACTTGAAAGTATTTC
GATTTCTTGGCTTTATATATCTTGTGGAAAGGACGAAACACCGAGGA
GCACCAGGAACTTGCCAGTTTAAGTACTCTGTGCTGGAAACAGCACA
GAATCTACTTAAACAAGGCAAAATGCCGTGTTTATCTCGTCAACTTG
TTGGCGAGATTTTTTCTAAAGCTTGAATTCAAGCTTGAATTCAGCTG
ACGTGCCTCGGACCGCTAGGAACCCCTAGTGATGGAGTTGGCCACTC
CCTCTCTGCGCGCTCGCTCGCTCACTGAGGCCGGGCGACCAAAGGTC
GCCCGACGCCCGGGCTTTGCCCGGGCGGCCTCAGTGAGCGAGCGAGC
GCGCAG
Exemplary pITR-CMV.hKCNQ4codop.U6-hsa408Rev
sequence
(SEQ ID NO: 270)
CTGCGCGCTCGCTCGCTCACTGAGGCCGCCCGGGCGTCGGGCGACCT
TTGGTCGCCCGGCCTCAGTGAGCGAGCGAGCGCGCAGAGAGGGAGTG
GCCAACTCCATCACTAGGGGTTCCTGCGGCCGCACGCGTCTAGATCC
CATATATGGAGTTCCGCGTTACATAACTTACGGTAAATGGCCCGCCT
GGCTGACCGCCCAACGACCCCCGCCCATTGACGTCAATAATGACGTA
TGTTCCCATAGTAACGCCAATAGGGACTTTCCATTGACGTCAATGGG
TGGAGTATTTACGGTAAACTGCCCACTTGGCAGTACATCAAGTGTAT
CATATGCCAAGTACGCCCCCTATTGACGTCAATGACGGTAAATGGCC
CGCCTGGCATTATGCCCAGTACATGACCTTATGGGACTTTCCTACTT
GGCAGTACATCTACGTATTAGTCATCGCTATTACCATGGTGATGCGG
TTTTGGCAGTACATCAATGGGCGTGGATAGCGGTTTGACTCACGGGG
ATTTCCAAGTCTCCACCCCATTGACGTCAATGGGAGTTTGTTTTGGC
ACCAAAATCAACGGGACTTTCCAAAATGTCGTAACAACTCCGCCCCA
TTGACGCAAATGGGCGGTAGGCGTGTACGGTGGGAGGTCTATATAAG
CAGAGCTCGTTTAGTGAACCGTCGTTTAGTGAACCGTcagatcgcct
ggagacgcACCGGTGCCACCATGGCTGAAGCCCCTCCTAGAAGGCTT
GGACTGGGACCTCCTCCTGGGGATGCTCCTAGAGCTGAACTGGTGGC
TCTGACAGCCGTGCAGTCTGAACAAGGCGAAGCTGGTGGCGGCGGAT
CTCCACGTAGACTTGGACTGCTGGGAAGCCCTCTTCCTCCTGGTGCT
CCACTTCCTGGACCTGGCAGTGGATCTGGATCTGCCTGTGGCCAGAG
AAGCTCTGCCGCTCACAAGAGATACCGGCGGCTGCAGAACTGGGTGT
ACAACGTGCTGGAAAGACCCAGAGGCTGGGCCTTCGTGTACCACGTG
TTCATCTTTCTGCTGGTGTTCAGCTGCCTGGTGCTGTCCGTGCTGAG
CACCATCCAAGAACATCAAGAGCTGGCTAACGAGTGCCTGTTAATAC
TGGAGTTTGTGATGATTGTGGTGTTCGGCCTCGAGTACATCGTCCGC
GTTTGGAGCGCCGGCTGCTGCTGCAGATATAGAGGTTGGCAAGGCAG
ATTCCGCTTCGCCAGAAAGCCCTTCTGCGTGATCGACTTCATCGTGT
TCGTGGCCAGCGTGGCCGTGATTGCTGCTGGCACACAGGGCAACATC
TTCGCCACAAGCGCCCTGCGGAGCATGCGGTTTCTGCAGATCCTGAG
AATGGTCCGAATGGACAGAAGAGGCGGCACCTGGAAGCTGCTGGGCT
CTGTGGTGTACGCCCACAGCAAAGAGCTGATCACCGCCTGGTACATC
GGATTTCTGGTGCTGATCTTCGCCTCCTTCCTGGTGTACCTGGCCGA
GAAGGACGCCAACAGCGACTTTAGCAGCTACGCCGACTCTCTTTGGT
GGGGCACCATCACACTGACCACCATCGGCTACGGCGACAAGACCCCT
CACACATGGCTGGGAAGAGTGCTGGCCGCTGGATTTGCTCTGCTGGG
CATCAGCTTTTTCGCCCTGCCTGCCGGAATCCTCGGATCTGGCTTTG
CCCTGAAGGTGCAAGAGCAGCACCGGCAGAAGCACTTCGAGAAGAGA
AGAATGCCTGCCGCCAACCTGATTCAGGCCGCTTGGAGACTGTACAG
CACCGACATGAGCAGAGCCTACCTGACCGCCACGTGGTATTATTACG
ACTCGATCCTGCCTAGCTTCCGCGAACTGGCCCTGCTGTTTGAGCAT
GTGCAGAGAGCCAGAAACGGCGGCCTCAGACCTCTGGAAGTTCGGAG
AGCACCTGTGCCTGATGGCGCCCCTTCTAGATATCCTCCAGTGGCCA
CCTGTCACAGACCCGGCAGCACATCTTTTTGCCCTGGCGAGTCTAGC
CGGATGGGCATCAAGGACAGAATCAGAATGGGCAGCAGCCAGCGGAG
AACAGGCCCTTCTAAACAGCATCTGGCCCCTCCAACCATGCCTACAA
GCCCTAGCTCTGAGCAAGTGGGCGAAGCCACCTCTCCTACCAAGGTG
CAGAAGTCCTGGTCCTTCAACGACCGGACCAGATTCAGAGCCAGCCT
GAGACTGAAGCCCAGAACCTCTGCCGAGGATGCCCCTTCTGAAGAGG
TGGCCGAAGAGAAGTCCTACCAGTGCGAGCTGACCGTGGACGACATC
ATGCCAGCCGTGAAAACCGTGATACGGTCTATCCGGATCCTGAAGTT
CCTGGTGGCCAAGCGGAAGTTCAAAGAGACACTGCGGCCCTACGACG
TGAAGGACGTGATCGAGCAGTATTCTGCCGGCCACCTGGACATGCTG
GGCAGAATCAAGAGCCTGCAGACCAGAGTGGACCAGATCGTTGGAAG
AGGCCCAGGCGACAGAAAGGCCAGAGAGAAGGGCGATAAGGGCCCAT
CTGATGCCGAGGTTGTCGACGAGATATCAATGATGGGCAGAGTGGTC
AAGGTGGAAAAACAGGTGCAGAGCATCGAGCACAAGCTGGACCTGCT
GCTGGGATTCTACAGCCGGTGTCTGAGAAGCGGCACATCTGCATCTC
TGGGCGCTGTGCAGGTCCCACTGTTCGACCCTGATATCACCAGCGAC
TATCACAGCCCCGTGGACCACGAGGACATCTCCGTTTCTGCTCAGAC
CCTGAGCATCAGCAGATCCGTGTCCACCAACATGGACTAATAAGAGC
TCGCTGATCAGCCTCGACTGTGCCTTCTAGTTGCCAGCCATCTGTTG
TTTGCCCCTCCCCCGTGCCTTCCTTGACCCTGGAAGGTGCCACTCCC
ACTGTCCTTTCCTAATAAAATGAGGAAATTGCATCGCATTGTCTGAG
TAGGTGTCATTCTATTCTGGGGGGTGGGGTGGGGCAGGACAGCAAGG
GGGAGGATTGGGAAGACAATAGCAGGCATGCTGGGGATGCGGTGGGC
TCTATGGTTAATTAAGGTCGGGCAGGAAGAGGGCCTATTTCCCATGA
TTCCTTCATATTTGCATATACGATACAAGGCTGTTAGAGAGATAATT
AGAATTAATTTGACTGTAAACACAAAGATATTAGTACAAAATACGTG
ACGTAGAAAGTAATAATTTCTTGGGTAGTTTGCAGTTTTAAAATTAT
GTTTTAAAATGGACTATCATATGCTTACCGTAACTTGAAAGTATTTC
GATTTCTTGGCTTTATATATCTTGTGGAAAGGACGAAACACCGAAGC
CGAAAACCACGATCATCGTTTAAGTACTCTGTGCTGGAAACAGCACA
GAATCTACTTAAACAAGGCAAAATGCCGTGTTTATCTCGTCAACTTG
TTGGCGAGATTTTTTCTAAAGCTTGAATTCAAGCTTGAATTCAGCTG
ACGTGCCTCGGACCGCTAGGAACCCCTAGTGATGGAGTTGGCCACTC
CCTCTCTGCGCGCTCGCTCGCTCACTGAGGCCGGGCGACCAAAGGTC
GCCCGACGCCCGGGCTTTGCCCGGGCGGCCTCAGTGAGCGAGCGAGC
GCGCAG
Exemplary pITR-CMV.hKCNQ4codop.U6-mmu408Rev
sequence
(SEQ ID NO: 271)
CTGCGCGCTCGCTCGCTCACTGAGGCCGCCCGGGCGTCGGGCGACCT
TTGGTCGCCCGGCCTCAGTGAGCGAGCGAGCGCGCAGAGAGGGAGTG
GCCAACTCCATCACTAGGGGTTCCTGCGGCCGCACGCGTCTAGATCC
CATATATGGAGTTCCGCGTTACATAACTTACGGTAAATGGCCCGCCT
GGCTGACCGCCCAACGACCCCCGCCCATTGACGTCAATAATGACGTA
TGTTCCCATAGTAACGCCAATAGGGACTTTCCATTGACGTCAATGGG
TGGAGTATTTACGGTAAACTGCCCACTTGGCAGTACATCAAGTGTAT
CATATGCCAAGTACGCCCCCTATTGACGTCAATGACGGTAAATGGCC
CGCCTGGCATTATGCCCAGTACATGACCTTATGGGACTTTCCTACTT
GGCAGTACATCTACGTATTAGTCATCGCTATTACCATGGTGATGCGG
TTTTGGCAGTACATCAATGGGCGTGGATAGCGGTTTGACTCACGGGG
ATTTCCAAGTCTCCACCCCATTGACGTCAATGGGAGTTTGTTTTGGC
ACCAAAATCAACGGGACTTTCCAAAATGTCGTAACAACTCCGCCCCA
TTGACGCAAATGGGCGGTAGGCGTGTACGGTGGGAGGTCTATATAAG
CAGAGCTCGTTTAGTGAACCGTCGTTTAGTGAACCGTcagatcgcct
ggagacgcACCGGTGCCACCATGGCTGAAGCCCCTCCTAGAAGGCTT
GGACTGGGACCTCCTCCTGGGGATGCTCCTAGAGCTGAACTGGTGGC
TCTGACAGCCGTGCAGTCTGAACAAGGCGAAGCTGGTGGCGGCGGAT
CTCCACGTAGACTTGGACTGCTGGGAAGCCCTCTTCCTCCTGGTGCT
CCACTTCCTGGACCTGGCAGTGGATCTGGATCTGCCTGTGGCCAGAG
AAGCTCTGCCGCTCACAAGAGATACCGGCGGCTGCAGAACTGGGTGT
ACAACGTGCTGGAAAGACCCAGAGGCTGGGCCTTCGTGTACCACGTG
TTCATCTTTCTGCTGGTGTTCAGCTGCCTGGTGCTGTCCGTGCTGAG
CACCATCCAAGAACATCAAGAGCTGGCTAACGAGTGCCTGTTAATAC
TGGAGTTTGTGATGATTGTGGTGTTCGGCCTCGAGTACATCGTCCGC
GTTTGGAGCGCCGGCTGCTGCTGCAGATATAGAGGTTGGCAAGGCAG
ATTCCGCTTCGCCAGAAAGCCCTTCTGCGTGATCGACTTCATCGTGT
TCGTGGCCAGCGTGGCCGTGATTGCTGCTGGCACACAGGGCAACATC
TTCGCCACAAGCGCCCTGCGGAGCATGCGGTTTCTGCAGATCCTGAG
AATGGTCCGAATGGACAGAAGAGGCGGCACCTGGAAGCTGCTGGGCT
CTGTGGTGTACGCCCACAGCAAAGAGCTGATCACCGCCTGGTACATC
GGATTTCTGGTGCTGATCTTCGCCTCCTTCCTGGTGTACCTGGCCGA
GAAGGACGCCAACAGCGACTTTAGCAGCTACGCCGACTCTCTTTGGT
GGGGCACCATCACACTGACCACCATCGGCTACGGCGACAAGACCCCT
CACACATGGCTGGGAAGAGTGCTGGCCGCTGGATTTGCTCTGCTGGG
CATCAGCTTTTTCGCCCTGCCTGCCGGAATCCTCGGATCTGGCTTTG
CCCTGAAGGTGCAAGAGCAGCACCGGCAGAAGCACTTCGAGAAGAGA
AGAATGCCTGCCGCCAACCTGATTCAGGCCGCTTGGAGACTGTACAG
CACCGACATGAGCAGAGCCTACCTGACCGCCACGTGGTATTATTACG
ACTCGATCCTGCCTAGCTTCCGCGAACTGGCCCTGCTGTTTGAGCAT
GTGCAGAGAGCCAGAAACGGCGGCCTCAGACCTCTGGAAGTTCGGAG
AGCACCTGTGCCTGATGGCGCCCCTTCTAGATATCCTCCAGTGGCCA
CCTGTCACAGACCCGGCAGCACATCTTTTTGCCCTGGCGAGTCTAGC
CGGATGGGCATCAAGGACAGAATCAGAATGGGCAGCAGCCAGCGGAG
AACAGGCCCTTCTAAACAGCATCTGGCCCCTCCAACCATGCCTACAA
GCCCTAGCTCTGAGCAAGTGGGCGAAGCCACCTCTCCTACCAAGGTG
CAGAAGTCCTGGTCCTTCAACGACCGGACCAGATTCAGAGCCAGCCT
GAGACTGAAGCCCAGAACCTCTGCCGAGGATGCCCCTTCTGAAGAGG
TGGCCGAAGAGAAGTCCTACCAGTGCGAGCTGACCGTGGACGACATC
ATGCCAGCCGTGAAAACCGTGATACGGTCTATCCGGATCCTGAAGTT
CCTGGTGGCCAAGCGGAAGTTCAAAGAGACACTGCGGCCCTACGACG
TGAAGGACGTGATCGAGCAGTATTCTGCCGGCCACCTGGACATGCTG
GGCAGAATCAAGAGCCTGCAGACCAGAGTGGACCAGATCGTTGGAAG
AGGCCCAGGCGACAGAAAGGCCAGAGAGAAGGGCGATAAGGGCCCAT
CTGATGCCGAGGTTGTCGACGAGATATCAATGATGGGCAGAGTGGTC
AAGGTGGAAAAACAGGTGCAGAGCATCGAGCACAAGCTGGACCTGCT
GCTGGGATTCTACAGCCGGTGTCTGAGAAGCGGCACATCTGCATCTC
TGGGCGCTGTGCAGGTCCCACTGTTCGACCCTGATATCACCAGCGAC
TATCACAGCCCCGTGGACCACGAGGACATCTCCGTTTCTGCTCAGAC
CCTGAGCATCAGCAGATCCGTGTCCACCAACATGGACTAATAAGAGC
TCGCTGATCAGCCTCGACTGTGCCTTCTAGTTGCCAGCCATCTGTTG
TTTGCCCCTCCCCCGTGCCTTCCTTGACCCTGGAAGGTGCCACTCCC
ACTGTCCTTTCCTAATAAAATGAGGAAATTGCATCGCATTGTCTGAG
TAGGTGTCATTCTATTCTGGGGGGTGGGGTGGGGCAGGACAGCAAGG
GGGAGGATTGGGAAGACAATAGCAGGCATGCTGGGGATGCGGTGGGC
TCTATGGTTAATTAAGGTCGGGCAGGAAGAGGGCCTATTTCCCATGA
TTCCTTCATATTTGCATATACGATACAAGGCTGTTAGAGAGATAATT
AGAATTAATTTGACTGTAAACACAAAGATATTAGTACAAAATACGTG
ACGTAGAAAGTAATAATTTCTTGGGTAGTTTGCAGTTTTAAAATTAT
GTTTTAAAATGGACTATCATATGCTTACCGTAACTTGAAAGTATTTC
GATTTCTTGGCTTTATATATCTTGTGGAAAGGACGAAACACCGAAGC
CAAAGACCACAATCATCGTTTAAGTACTCTGTGCTGGAAACAGCACA
GAATCTACTTAAACAAGGCAAAATGCCGTGTTTATCTCGTCAACTTG
TTGGCGAGATTTTTTCTAAAGCTTGAATTCAAGCTTGAATTCAGCTG
ACGTGCCTCGGACCGCTAGGAACCCCTAGTGATGGAGTTGGCCACTC
CCTCTCTGCGCGCTCGCTCGCTCACTGAGGCCGGGCGACCAAAGGTC
GCCCGACGCCCGGGCTTTGCCCGGGCGGCCTCAGTGAGCGAGCGAGC
GCGCAG
Exemplary pITR-CMV.hKCNQ4codop.U6-hsammu386Fw
sequence
(SEQ ID NO: 272)
CCTGCAGGCAGCTGCGCGCTCGCTCGCTCACTGAGGCCGCCCGGGCG
TCGGGCGACCTTTGGTCGCCCGGCCTCAGTGAGCGAGCGAGCGCGCA
GAGAGGGAGTGGCCAACTCCATCACTAGGGGTTCCTGCGGCCGCACG
CGTCTAGATCCCATATATGGAGTTCCGCGTTACATAACTTACGGTAA
ATGGCCCGCCTGGCTGACCGCCCAACGACCCCCGCCCATTGACGTCA
ATAATGACGTATGTTCCCATAGTAACGCCAATAGGGACTTTCCATTG
ACGTCAATGGGTGGAGTATTTACGGTAAACTGCCCACTTGGCAGTAC
ATCAAGTGTATCATATGCCAAGTACGCCCCCTATTGACGTCAATGAC
GGTAAATGGCCCGCCTGGCATTATGCCCAGTACATGACCTTATGGGA
CTTTCCTACTTGGCAGTACATCTACGTATTAGTCATCGCTATTACCA
TGGTGATGCGGTTTTGGCAGTACATCAATGGGCGTGGATAGCGGTTT
GACTCACGGGGATTTCCAAGTCTCCACCCCATTGACGTCAATGGGAG
TTTGTTTTGGCACCAAAATCAACGGGACTTTCCAAAATGTCGTAACA
ACTCCGCCCCATTGACGCAAATGGGCGGTAGGCGTGTACGGTGGGAG
GTCTATATAAGCAGAGCTCGTTTAGTGAACCGTCGTTTAGTGAACCG
TCAGATCGCCTGGAGACGCACCGGTGCCACCATGGCTGAAGCCCCTC
CTAGAAGGCTTGGACTGGGACCTCCTCCTGGGGATGCTCCTAGAGCT
GAACTGGTGGCTCTGACAGCCGTGCAGTCTGAACAAGGCGAAGCTGG
TGGCGGCGGATCTCCACGTAGACTTGGACTGCTGGGAAGCCCTCTTC
CTCCTGGTGCTCCACTTCCTGGACCTGGCAGTGGATCTGGATCTGCC
TGTGGCCAGAGAAGCTCTGCCGCTCACAAGAGATACCGGCGGCTGCA
GAACTGGGTGTACAACGTGCTGGAAAGACCCAGAGGCTGGGCCTTCG
TGTACCACGTGTTCATCTTTCTGCTGGTGTTCAGCTGCCTGGTGCTG
TCCGTGCTGAGCACCATCCAAGAACATCAAGAGCTGGCTAACGAGTG
CCTGTTAATACTGGAGTTTGTGATGATTGTGGTGTTCGGCCTCGAGT
ACATCGTCCGCGTTTGGAGCGCCGGCTGCTGCTGCAGATATAGAGGT
TGGCAAGGCAGATTCCGCTTCGCCAGAAAGCCCTTCTGCGTGATCGA
CTTCATCGTGTTCGTGGCCAGCGTGGCCGTGATTGCTGCTGGCACAC
AGGGCAACATCTTCGCCACAAGCGCCCTGCGGAGCATGCGGTTTCTG
CAGATCCTGAGAATGGTCCGAATGGACAGAAGAGGCGGCACCTGGAA
GCTGCTGGGCTCTGTGGTGTACGCCCACAGCAAAGAGCTGATCACCG
CCTGGTACATCGGATTTCTGGTGCTGATCTTCGCCTCCTTCCTGGTG
TACCTGGCCGAGAAGGACGCCAACAGCGACTTTAGCAGCTACGCCGA
CTCTCTTTGGTGGGGCACCATCACACTGACCACCATCGGCTACGGCG
ACAAGACCCCTCACACATGGCTGGGAAGAGTGCTGGCCGCTGGATTT
GCTCTGCTGGGCATCAGCTTTTTCGCCCTGCCTGCCGGAATCCTCGG
ATCTGGCTTTGCCCTGAAGGTGCAAGAGCAGCACCGGCAGAAGCACT
TCGAGAAGAGAAGAATGCCTGCCGCCAACCTGATTCAGGCCGCTTGG
AGACTGTACAGCACCGACATGAGCAGAGCCTACCTGACCGCCACGTG
GTATTATTACGACTCGATCCTGCCTAGCTTCCGCGAACTGGCCCTGC
TGTTTGAGCATGTGCAGAGAGCCAGAAACGGCGGCCTCAGACCTCTG
GAAGTTCGGAGAGCACCTGTGCCTGATGGCGCCCCTTCTAGATATCC
TCCAGTGGCCACCTGTCACAGACCCGGCAGCACATCTTTTTGCCCTG
GCGAGTCTAGCCGGATGGGCATCAAGGACAGAATCAGAATGGGCAGC
AGCCAGCGGAGAACAGGCCCTTCTAAACAGCATCTGGCCCCTCCAAC
CATGCCTACAAGCCCTAGCTCTGAGCAAGTGGGCGAAGCCACCTCTC
CTACCAAGGTGCAGAAGTCCTGGTCCTTCAACGACCGGACCAGATTC
AGAGCCAGCCTGAGACTGAAGCCCAGAACCTCTGCCGAGGATGCCCC
TTCTGAAGAGGTGGCCGAAGAGAAGTCCTACCAGTGCGAGCTGACCG
TGGACGACATCATGCCAGCCGTGAAAACCGTGATACGGTCTATCCGG
ATCCTGAAGTTCCTGGTGGCCAAGCGGAAGTTCAAAGAGACACTGCG
GCCCTACGACGTGAAGGACGTGATCGAGCAGTATTCTGCCGGCCACC
TGGACATGCTGGGCAGAATCAAGAGCCTGCAGACCAGAGTGGACCAG
ATCGTTGGAAGAGGCCCAGGCGACAGAAAGGCCAGAGAGAAGGGCGA
TAAGGGCCCATCTGATGCCGAGGTTGTCGACGAGATATCAATGATGG
GCAGAGTGGTCAAGGTGGAAAAACAGGTGCAGAGCATCGAGCACAAG
CTGGACCTGCTGCTGGGATTCTACAGCCGGTGTCTGAGAAGCGGCAC
ATCTGCATCTCTGGGCGCTGTGCAGGTCCCACTGTTCGACCCTGATA
TCACCAGCGACTATCACAGCCCCGTGGACCACGAGGACATCTCCGTT
TCTGCTCAGACCCTGAGCATCAGCAGATCCGTGTCCACCAACATGGA
CTAATAAGAGCTCGCTGATCAGCCTCGACTGTGCCTTCTAGTTGCCA
GCCATCTGTTGTTTGCCCCTCCCCCGTGCCTTCCTTGACCCTGGAAG
GTGCCACTCCCACTGTCCTTTCCTAATAAAATGAGGAAATTGCATCG
CATTGTCTGAGTAGGTGTCATTCTATTCTGGGGGGTGGGGTGGGGCA
GGACAGCAAGGGGGAGGATTGGGAAGACAATAGCAGGCATGCTGGGG
ATGCGGTGGGCTCTATGGTTAATTAAGGTCGGGCAGGAAGAGGGCCT
ATTTCCCATGATTCCTTCATATTTGCATATACGATACAAGGCTGTTA
GAGAGATAATTAGAATTAATTTGAGTGTAAACACAAAGATATTAGTA
GAAAATACGTGACGTAGAAAGTAATAATTTCTTGGGTAGTTTGCAGT
TTTAAAATTATGTTTTAAAATGGACTATCATATGCTTACCGTAACTT
GAAAGTATTTCGATTTCTTGGCTTTATATATCTTGTGGAAAGGACGA
AACACCGAGGAGCACCAGGAACTTGCCAGTTTAAGTACTCTGTGCTG
GAAACAGCACAGAATCTACTTAAACAAGGCAAAATGCCGTGTTTATC
TCGTCAACTTGTTGGCGAGATTTTTTCTAAAGCTTGAATTCAAGCTT
GAATTCAGCTGACGTGCCTCGGACCGCTAGGAACCCCTAGTGATGGA
GTTGGCCACTCCCTCTCTGCGCGCTCGCTCGCTCACTGAGGCCGGGC
GACCAAAGGTCGCCCGACGCCCGGGCTTTGCCCGGGCGGCCTCAGTG
AGCGAGCGAGCGCGCAGCTGCCTGCAGGGGCGCCTGATGCGGTATTT
TCTCCTTACGCATCTGTGCGGTATTTCACACCGCATACGTCAAAGCA
ACCATAGTACGCGCCCTGTAGCGGCGCATTAAGCGCGGCGGGTGTGG
TGGTTACGCGCAGCGTGACCGCTACACTTGCCAGCGCCCTAGCGCCC
GCTCCTTTCGCTTTCTTCCCTTCCTTTCTCGCCACGTTCGCCGGCTT
TCCCCGTCAAGCTCTAAATCGGGGGCTCCCTTTAGGGTTCCGATTTA
GTGCTTTACGGCACCTCGACCCCAAAAAACTTGATTTGGGTGATGGT
TCACGTAGTGGGCCATCGCCCTGATAGACGGTTTTTCGCCCTTTGAC
GTTGGAGTCCACGTTCTTTAATAGTGGACTCTTGTTCCAAACTGGAA
CAACACTCAACCCTATCTCGGGCTATTCTTTTGATTTATAAGGGATT
TTGCCGATTTCGGCCTATTGGTTAAAAAATGAGCTGATTTAACAAAA
ATTTAACGCGAATTTTAACAAAATATTAACGTTTACAATTTTATGGT
GCACTCTCAGTACAATCTGCTCTGATGCCGCATAGTTAAGCCAGCCC
CGACACCCGCCAACACCCGCTGACGCGCCCTGACGGGCTTGTCTGCT
CCCGGCATCCGCTTACAGACAAGCTGTGACCGTCTCCGGGAGCTGCA
TGTGTCAGAGGTTTTCACCGTCATCACCGAAACGCGCGAGACGAAAG
GGCCTCGTGATACGCCTATTTTTATAGGTTAATGTCATGAACAATAA
AACTGTCTGCTTACATAAACAGTAATACAAGGGGTGTTATGAGCCAT
ATTCAACGGGAAACGTCGAGGCCGCGATTAAATTCCAACATGGATGC
TGATTTATATGGGTATAAATGGGCTCGCGATAATGTCGGGCAATCAG
GTGCGACAATCTATCGCTTGTATGGGAAGCCCGATGCGCCAGAGTTG
TTTCTGAAACATGGCAAAGGTAGCGTTGCCAATGATGTTACAGATGA
GATGGTCAGACTAAACTGGCTGACGGAATTTATGCCTCTTCCGACCA
TCAAGCATTTTATCCGTACTCCTGATGATGCATGGTTACTCACCACT
GCGATCCCCGGAAAAACAGCATTCCAGGTATTAGAAGAATATCCTGA
TTCAGGTGAAAATATTGTTGATGCGCTGGCAGTGTTCCTGCGCCGGT
TGCATTCGATTCCTGTTTGTAATTGTCCTTTTAACAGCGATCGCGTA
TTTCGTCTCGCTCAGGCGCAATCACGAATGAATAACGGTTTGGTTGA
TGCGAGTGATTTTGATGACGAGCGTAATGGCTGGCCTGTTGAACAAG
TCTGGAAAGAAATGCATAAACTTTTGCCATTCTCACCGGATTCAGTC
GTCACTCATGGTGATTTCTCACTTGATAACCTTATTTTTGACGAGGG
GAAATTAATAGGTTGTATTGATGTTGGACGAGTCGGAATCGCAGACC
GATACCAGGATCTTGCCATCCTATGGAACTGCCTCGGTGAGTTTTCT
CCTTCATTACAGAAACGGCTTTTTCAAAAATATGGTATTGATAATCC
TGATATGAATAAATTGCAGTTTCATTTGATGCTCGATGAGTTTTTCT
AATCTCATGACCAAAATCCCTTAACGTGAGTTTTCGTTCCACTGAGC
GTCAGACCCCGTAGAAAAGATCAAAGGATCTTCTTGAGATCCTTTTT
TTCTGCGCGTAATCTGCTGCTTGCAAACAAAAAAACCACCGCTACCA
GCGGTGGTTTGTTTGCCGGATCAAGAGCTACCAACTCTTTTTCCGAA
GGTAACTGGCTTCAGCAGAGCGCAGATACCAAATACTGTCCTTCTAG
TGTAGCCGTAGTTAGGCCACCACTTCAAGAACTCTGTAGCACCGCCT
ACATACCTCGCTCTGCTAATCCTGTTACCAGTGGCTGCTGCCAGTGG
CGATAAGTCGTGTCTTACCGGGTTGGACTCAAGACGATAGTTACCGG
ATAAGGCGCAGCGGTCGGGCTGAACGGGGGGTTCGTGCACACAGCCC
AGCTTGGAGCGAACGACCTACACCGAACTGAGATACCTACAGCGTGA
GCTATGAGAAAGCGCCACGCTTCCCGAAGGGAGAAAGGCGGACAGGT
ATCCGGTAAGCGGCAGGGTCGGAACAGGAGAGCGCACGAGGGAGCTT
CCAGGGGGAAACGCCTGGTATCTTTATAGTCCTGTCGGGTTTCGCCA
CCTCTGACTTGAGCGTCGATTTTTGTGATGCTCGTCAGGGGGGCGGA
GCCTATGGAAAAACGCCAGCAACGCGGCCTTTTTACGGTTCCTGGCC
TTTTGCTGGCCTTTTGCTCACATGT
Exemplary pITR-CMV.hKCNQ4codop.U6-hsa408Rev
sequence
(SEQ ID NO: 273)
CCTGCAGGCAGCTGCGCGCTCGCTCGCTCACTGAGGCCGCCCGGGCG
TCGGGCGACCTTTGGTCGCCCGGCCTCAGTGAGCGAGCGAGCGCGCA
GAGAGGGAGTGGCCAACTCCATCACTAGGGGTTCCTGCGGCCGCACG
CGTCTAGATCCCATATATGGAGTTCCGCGTTACATAACTTACGGTAA
ATGGCCCGCCTGGCTGACCGCCCAACGACCCCCGCCCATTGACGTCA
ATAATGACGTATGTTCCCATAGTAACGCCAATAGGGACTTTCCATTG
ACGTCAATGGGTGGAGTATTTACGGTAAACTGCCCACTTGGCAGTAC
ATCAAGTGTATCATATGCCAAGTACGCCCCCTATTGACGTCAATGAC
GGTAAATGGCCCGCCTGGCATTATGCCCAGTACATGACCTTATGGGA
CTTTCCTACTTGGCAGTACATCTACGTATTAGTCATCGCTATTACCA
TGGTGATGCGGTTTTGGCAGTACATCAATGGGCGTGGATAGCGGTTT
GACTCACGGGGATTTCCAAGTCTCCACCCCATTGACGTCAATGGGAG
TTTGTTTTGGCACCAAAATCAACGGGACTTTCCAAAATGTCGTAACA
ACTCCGCCCCATTGACGCAAATGGGCGGTAGGCGTGTACGGTGGGAG
GTCTATATAAGCAGAGCTCGTTTAGTGAACCGTCGTTTAGTGAACCG
TcagatcgcctggagacgcACCGGTGCCACCATGGCTGAAGCCCCTC
CTAGAAGGCTTGGACTGGGACCTCCTCCTGGGGATGCTCCTAGAGCT
GAACTGGTGGCTCTGACAGCCGTGCAGTCTGAACAAGGCGAAGCTGG
TGGCGGCGGATCTCCACGTAGACTTGGACTGCTGGGAAGCCCTCTTC
CTCCTGGTGCTCCACTTCCTGGACCTGGCAGTGGATCTGGATCTGCC
TGTGGCCAGAGAAGCTCTGCCGCTCACAAGAGATACCGGCGGCTGCA
GAACTGGGTGTACAACGTGCTGGAAAGACCCAGAGGCTGGGCCTTCG
TGTACCACGTGTTCATCTTTCTGCTGGTGTTCAGCTGCCTGGTGCTG
TCCGTGCTGAGCACCATCCAAGAACATCAAGAGCTGGCTAACGAGTG
CCTGTTAATACTGGAGTTTGTGATGATTGTGGTGTTCGGCCTCGAGT
ACATCGTCCGCGTTTGGAGCGCCGGCTGCTGCTGCAGATATAGAGGT
TGGCAAGGCAGATTCCGCTTCGCCAGAAAGCCCTTCTGCGTGATCGA
CTTCATCGTGTTCGTGGCCAGCGTGGCCGTGATTGCTGCTGGCACAC
AGGGCAACATCTTCGCCACAAGCGCCCTGCGGAGCATGCGGTTTCTG
CAGATCCTGAGAATGGTCCGAATGGACAGAAGAGGCGGCACCTGGAA
GCTGCTGGGCTCTGTGGTGTACGCCCACAGCAAAGAGCTGATCACCG
CCTGGTACATCGGATTTCTGGTGCTGATCTTCGCCTCCTTCCTGGTG
TACCTGGCCGAGAAGGACGCCAACAGCGACTTTAGCAGCTACGCCGA
CTCTCTTTGGTGGGGCACCATCACACTGACCACCATCGGCTACGGCG
ACAAGACCCCTCACACATGGCTGGGAAGAGTGCTGGCCGCTGGATTT
GCTCTGCTGGGCATCAGCTTTTTCGCCCTGCCTGCCGGAATCCTCGG
ATCTGGCTTTGCCCTGAAGGTGCAAGAGCAGCACCGGCAGAAGCACT
TCGAGAAGAGAAGAATGCCTGCCGCCAACCTGATTCAGGCCGCTTGG
AGACTGTACAGCACCGACATGAGCAGAGCCTACCTGACCGCCACGTG
GTATTATTACGACTCGATCCTGCCTAGCTTCCGCGAACTGGCCCTGC
TGTTTGAGCATGTGCAGAGAGCCAGAAACGGCGGCCTCAGACCTCTG
GAAGTTCGGAGAGCACCTGTGCCTGATGGCGCCCCTTCTAGATATCC
TCCAGTGGCCACCTGTCACAGACCCGGCAGCACATCTTTTTGCCCTG
GCGAGTCTAGCCGGATGGGCATCAAGGACAGAATCAGAATGGGCAGC
AGCCAGCGGAGAACAGGCCCTTCTAAACAGCATCTGGCCCCTCCAAC
CATGCCTACAAGCCCTAGCTCTGAGCAAGTGGGCGAAGCCACCTCTC
CTACCAAGGTGCAGAAGTCCTGGTCCTTCAACGACCGGACCAGATTC
AGAGCCAGCCTGAGACTGAAGCCCAGAACCTCTGCCGAGGATGCCCC
TTCTGAAGAGGTGGCCGAAGAGAAGTCCTACCAGTGCGAGCTGACCG
TGGACGACATCATGCCAGCCGTGAAAACCGTGATACGGTCTATCCGG
ATCCTGAAGTTCCTGGTGGCCAAGCGGAAGTTCAAAGAGACACTGCG
GCCCTACGACGTGAAGGACGTGATCGAGCAGTATTCTGCCGGCCACC
TGGACATGCTGGGCAGAATCAAGAGCCTGCAGACCAGAGTGGACCAG
ATCGTTGGAAGAGGCCCAGGCGACAGAAAGGCCAGAGAGAAGGGCGA
TAAGGGCCCATCTGATGCCGAGGTTGTCGACGAGATATCAATGATGG
GCAGAGTGGTCAAGGTGGAAAAACAGGTGCAGAGCATCGAGCACAAG
CTGGACCTGCTGCTGGGATTCTACAGCCGGTGTCTGAGAAGCGGCAC
ATCTGCATCTCTGGGCGCTGTGCAGGTCCCACTGTTCGACCCTGATA
TCACCAGCGACTATCACAGCCCCGTGGACCACGAGGACATCTCCGTT
TCTGCTCAGACCCTGAGCATCAGCAGATCCGTGTCCACCAACATGGA
CTAATAAGAGCTCGCTGATCAGCCTCGACTGTGCCTTCTAGTTGCCA
GCCATCTGTTGTTTGCCCCTCCCCCGTGCCTTCCTTGACCCTGGAAG
GTGCCACTCCCACTGTCCTTTCCTAATAAAATGAGGAAATTGCATCG
CATTGTCTGAGTAGGTGTCATTCTATTCTGGGGGGTGGGGTGGGGCA
GGACAGCAAGGGGGAGGATTGGGAAGACAATAGCAGGCATGCTGGGG
ATGCGGTGGGCTCTATGGTTAATTAAGGTCGGGCAGGAAGAGGGCCT
ATTTCCCATGATTCCTTCATATTTGCATATACGATACAAGGCTGTTA
GAGAGATAATTAGAATTAATTTGAGTGTAAACACAAAGATATTAGTA
GAAAATACGTGACGTAGAAAGTAATAATTTCTTGGGTAGTTTGCAGT
TTTAAAATTATGTTTTAAAATGGACTATCATATGCTTACCGTAACTT
GAAAGTATTTCGATTTCTTGGCTTTATATATCTTGTGGAAAGGACGA
AACACCGAAGCCGAAAACCACGATCATCGTTTAAGTACTCTGTGCTG
GAAACAGCACAGAATCTACTTAAACAAGGCAAAATGCCGTGTTTATC
TCGTCAACTTGTTGGCGAGATTTTTTCTAAAGCTTGAATTCAAGCTT
GAATTCAGCTGACGTGCCTCGGACCGCTAGGAACCCCTAGTGATGGA
GTTGGCCACTCCCTCTCTGCGCGCTCGCTCGCTCACTGAGGCCGGGC
GACCAAAGGTCGCCCGACGCCCGGGCTTTGCCCGGGCGGCCTCAGTG
AGCGAGCGAGCGCGCAGCTGCCTGCAGGGGCGCCTGATGCGGTATTT
TCTCCTTACGCATCTGTGCGGTATTTCACACCGCATACGTCAAAGCA
ACCATAGTACGCGCCCTGTAGCGGCGCATTAAGCGCGGCGGGTGTGG
TGGTTACGCGCAGCGTGACCGCTACACTTGCCAGCGCCCTAGCGCCC
GCTCCTTTCGCTTTCTTCCCTTCCTTTCTCGCCACGTTCGCCGGCTT
TCCCCGTCAAGCTCTAAATCGGGGGCTCCCTTTAGGGTTCCGATTTA
GTGCTTTACGGCACCTCGACCCCAAAAAACTTGATTTGGGTGATGGT
TCACGTAGTGGGCCATCGCCCTGATAGACGGTTTTTCGCCCTTTGAC
GTTGGAGTCCACGTTCTTTAATAGTGGACTCTTGTTCCAAACTGGAA
CAACACTCAACCCTATCTCGGGCTATTCTTTTGATTTATAAGGGATT
TTGCCGATTTCGGCCTATTGGTTAAAAAATGAGCTGATTTAACAAAA
ATTTAACGCGAATTTTAACAAAATATTAACGTTTACAATTTTATGGT
GCACTCTCAGTACAATCTGCTCTGATGCCGCATAGTTAAGCCAGCCC
CGACACCCGCCAACACCCGCTGACGCGCCCTGACGGGCTTGTCTGCT
CCCGGCATCCGCTTACAGACAAGCTGTGACCGTCTCCGGGAGCTGCA
TGTGTCAGAGGTTTTCACCGTCATCACCGAAACGCGCGAGACGAAAG
GGCCTCGTGATACGCCTATTTTTATAGGTTAATGTCATGAACAATAA
AACTGTCTGCTTACATAAACAGTAATACAAGGGGTGTTATGAGCCAT
ATTCAACGGGAAACGTCGAGGCCGCGATTAAATTCCAACATGGATGC
TGATTTATATGGGTATAAATGGGCTCGCGATAATGTCGGGCAATCAG
GTGCGACAATCTATCGCTTGTATGGGAAGCCCGATGCGCCAGAGTTG
TTTCTGAAACATGGCAAAGGTAGCGTTGCCAATGATGTTACAGATGA
GATGGTCAGACTAAACTGGCTGACGGAATTTATGCCTCTTCCGACCA
TCAAGCATTTTATCCGTACTCCTGATGATGCATGGTTACTCACCACT
GCGATCCCCGGAAAAACAGCATTCCAGGTATTAGAAGAATATCCTGA
TTCAGGTGAAAATATTGTTGATGCGCTGGCAGTGTTCCTGCGCCGGT
TGCATTCGATTCCTGTTTGTAATTGTCCTTTTAACAGCGATCGCGTA
TTTCGTCTCGCTCAGGCGCAATCACGAATGAATAACGGTTTGGTTGA
TGCGAGTGATTTTGATGACGAGCGTAATGGCTGGCCTGTTGAACAAG
TCTGGAAAGAAATGCATAAACTTTTGCCATTCTCACCGGATTCAGTC
GTCACTCATGGTGATTTCTCACTTGATAACCTTATTTTTGACGAGGG
GAAATTAATAGGTTGTATTGATGTTGGACGAGTCGGAATCGCAGACC
GATACCAGGATCTTGCCATCCTATGGAACTGCCTCGGTGAGTTTTCT
CCTTCATTACAGAAACGGCTTTTTCAAAAATATGGTATTGATAATCC
TGATATGAATAAATTGCAGTTTCATTTGATGCTCGATGAGTTTTTCT
AATCTCATGACCAAAATCCCTTAACGTGAGTTTTCGTTCCACTGAGC
GTCAGACCCCGTAGAAAAGATCAAAGGATCTTCTTGAGATCCTTTTT
TTCTGCGCGTAATCTGCTGCTTGCAAACAAAAAAACCACCGCTACCA
GCGGTGGTTTGTTTGCCGGATCAAGAGCTACCAACTCTTTTTCCGAA
GGTAACTGGCTTCAGCAGAGCGCAGATACCAAATACTGTCCTTCTAG
TGTAGCCGTAGTTAGGCCACCACTTCAAGAACTCTGTAGCACCGCCT
ACATACCTCGCTCTGCTAATCCTGTTACCAGTGGCTGCTGCCAGTGG
CGATAAGTCGTGTCTTACCGGGTTGGACTCAAGACGATAGTTACCGG
ATAAGGCGCAGCGGTCGGGCTGAACGGGGGGTTCGTGCACACAGCCC
AGCTTGGAGCGAACGACCTACACCGAACTGAGATACCTACAGCGTGA
GCTATGAGAAAGCGCCACGCTTCCCGAAGGGAGAAAGGCGGACAGGT
ATCCGGTAAGCGGCAGGGTCGGAACAGGAGAGCGCACGAGGGAGCTT
CCAGGGGGAAACGCCTGGTATCTTTATAGTCCTGTCGGGTTTCGCCA
CCTCTGACTTGAGCGTCGATTTTTGTGATGCTCGTCAGGGGGGCGGA
GCCTATGGAAAAACGCCAGCAACGCGGCCTTTTTACGGTTCCTGGCC
TTTTGCTGGCCTTTTGCTCACATGT
Exemplary pITR-CAG.hKCNQ4codop.U6-hsammu386Fw
sequence
(SEQ ID NO: 274)
CTGCGCGCTCGCTCGCTCACTGAGGCCGCCCGGGCGTCGGGCGACCT
TTGGTCGCCCGGCCTCAGTGAGCGAGCGAGCGCGCAGAGAGGGAGTG
GCCAACTCCATCACTAGGGGTTCCTGCGGCCGCACGCGTGACATTGA
TTATTGACTAGTTATTAATAGTAATCAATTACGGGGTCATTAGTTCA
TAGCCCATATATGGAGTTCCGCGTTACATAACTTACGGTAAATGGCC
CGCCTGGCTGACCGCCCAACGACCCCCGCCCATTGACGTCAATAATG
ACGTATGTTCCCATAGTAACGCCAATAGGGACTTTCCATTGACGTCA
ATGGGTGGACTATTTACGGTAAACTGCCCACTTGGCAGTACATCAAG
TGTATCATATGCCAAGTACGCCCCCTATTGACGTCAATGACGGTAAA
TGGCCCGCCTGGCATTATGCCCAGTACATGACCTTATGGGACTTTCC
TACTTGGCAGTACATCTACGTATTAGTCATCGCTATTACCATGGGTC
GAGGTGAGCCCCACGTTCTGCTTCACTCTCCCCATCTCCCCCCCCTC
CCCACCCCCAATTTTGTATTTATTTATTTTTTAATTATTTTGTGCAG
CGATGGGGGCGGGGGGGGGGGGGGCGCGCGCCAGGCGGGGCGGGGCG
GGGCGAGGGGCGGGGCGGGGCGAGGCGGAGAGGTGCGGCGGCAGCCA
ATCAGAGCGGCGCGCTCCGAAAGTTTCCTTTTATGGCGAGGCGGCGG
CGGCGGCGGCCCTATAAAAAGCGAAGCGCGCGGCGGGCGGGAGTCGC
TGCGTTGCCTTCGCCCCGTGCCCCGCTCCGCGCCGCCTCGCGCCGCC
CGCCCCGGCTCTGACTGACCGCGTTACTCCCACAGGTGAGCGGGCGG
GACGGCCCTTCTCCTCCGGGCTGTAATTAGCGCTTGGTTTAATGACG
GCTCGTTTCTTTTCTGTGGCTGCGTGAAAGCCTTAAAGGGCTCCGGG
AGGGCCCTTTGTGCGGGGGGGAGCGGCTCGGGGGGTGCGTGCGTGTG
TGTGTGCGTGGGGAGCGCCGCGTGCGGCCCGCGCTGCCCGGCGGCTG
TGAGCGCTGCGGGCGCGGCGCGGGGCTTTGTGCGCTCCGCGTGTGCG
CGAGGGGAGCGCGGCCGGGGGCGGTGCCCCGCGGTGCGGGGGGGCTG
CGAGGGGAACAAAGGCTGCGTGCGGGGTGTGTGCGTGGGGGGGTGAG
CAGGGGGTGTGGGCGCGGCGGTCGGGCTGTAACCCCCCCCTGCACCC
CCCTCCCCGAGTTGCTGAGCACGGCCCGGCTTCGGGTGCGGGGCTCC
GTGCGGGGCGTGGCGCGGGGCTCGCCGTGCCGGGCGGGGGGTGGCGG
CAGGTGGGGGTGCCGGGCGGGGCGGGGCCGCCTCGGGCCGGGGAGGG
CTCGGGGGAGGGGCGCGGCGGCCCCCGGAGCGCCGGCGGCTGTCGAG
GCGCGGCGAGCCGCAGCCATTGCCTTTTATGGTAATCGTGCGAGAGG
GCGCAGGGACTTCCTTTGTCCCAAATCTGTGCGGAGCCGAAATCTGG
GAGGCGCCGCCGCACCCCCTCTAGCGGGCGCGGGGCGAAGCGGTGCG
GCGCCGGCAGGAAGGAAATGGGCGGGGAGGGCCTTCGTGCGTCGCCG
CGCCGCCGTCCCCTTCTCCCTCTCCAGCCTCGGGGCTGTCCGCGGGG
GGACGGCTGCCTTCGGGGGGGACGGGGCAGGGCGGGGTTCGGCTTCT
GGCGTGTGACCGGCGGCTCTAGAGCCTCTGCTAACCATGTTCATGCC
TTCTTCTTTTTCCTACAGCTCCTGGGCAACGTGCTGGTTATTGTGAC
CGGTCGCTAGCCACCATGGCTGAAGCCCCTCCTAGAAGGCTTGGACT
GGGACCTCCTCCTGGGGATGCTCCTAGAGCTGAACTGGTGGCTCTGA
CAGCCGTGCAGTCTGAACAAGGCGAAGCTGGTGGCGGCGGATCTCCA
CGTAGACTTGGACTGCTGGGAAGCCCTCTTCCTCCTGGTGCTCCACT
TCCTGGACCTGGCAGTGGATCTGGATCTGCCTGTGGCCAGAGAAGCT
CTGCCGCTCACAAGAGATACCGGCGGCTGCAGAACTGGGTGTACAAC
GTGCTGGAAAGACCCAGAGGCTGGGCCTTCGTGTACCACGTGTTCAT
CTTTCTGCTGGTGTTCAGCTGCCTGGTGCTGTCCGTGCTGAGCACCA
TCCAAGAACATCAAGAGCTGGCTAACGAGTGCCTGTTAATACTGGAG
TTTGTGATGATTGTGGTGTTCGGCCTCGAGTACATCGTCCGCGTTTG
GAGCGCCGGCTGCTGCTGCAGATATAGAGGTTGGCAAGGCAGATTCC
GCTTCGCCAGAAAGCCCTTCTGCGTGATCGACTTCATCGTGTTCGTG
GCCAGCGTGGCCGTGATTGCTGCTGGCACACAGGGCAACATCTTCGC
CACAAGCGCCCTGCGGAGCATGCGGTTTCTGCAGATCCTGAGAATGG
TCCGAATGGACAGAAGAGGCGGCACCTGGAAGCTGCTGGGCTCTGTG
GTGTACGCCCACAGCAAAGAGCTGATCACCGCCTGGTACATCGGATT
TCTGGTGCTGATCTTCGCCTCCTTCCTGGTGTACCTGGCCGAGAAGG
ACGCCAACAGCGACTTTAGCAGCTACGCCGACTCTCTTTGGTGGGGC
ACCATCACACTGACCACCATCGGCTACGGCGACAAGACCCCTCACAC
ATGGCTGGGAAGAGTGCTGGCCGCTGGATTTGCTCTGCTGGGCATCA
GCTTTTTCGCCCTGCCTGCCGGAATCCTCGGATCTGGCTTTGCCCTG
AAGGTGCAAGAGCAGCACCGGCAGAAGCACTTCGAGAAGAGAAGAAT
GCCTGCCGCCAACCTGATTCAGGCCGCTTGGAGACTGTACAGCACCG
ACATGAGCAGAGCCTACCTGACCGCCACGTGGTATTATTACGACTCG
ATCCTGCCTAGCTTCCGCGAACTGGCCCTGCTGTTTGAGCATGTGCA
GAGAGCCAGAAACGGCGGCCTCAGACCTCTGGAAGTTCGGAGAGCAC
CTGTGCCTGATGGCGCCCCTTCTAGATATCCTCCAGTGGCCACCTGT
CACAGACCCGGCAGCACATCTTTTTGCCCTGGCGAGTCTAGCCGGAT
GGGCATCAAGGACAGAATCAGAATGGGCAGCAGCCAGCGGAGAACAG
GCCCTTCTAAACAGCATCTGGCCCCTCCAACCATGCCTACAAGCCCT
AGCTCTGAGCAAGTGGGCGAAGCCACCTCTCCTACCAAGGTGCAGAA
GTCCTGGTCCTTCAACGACCGGACCAGATTCAGAGCCAGCCTGAGAC
TGAAGCCCAGAACCTCTGCCGAGGATGCCCCTTCTGAAGAGGTGGCC
GAAGAGAAGTCCTACCAGTGCGAGCTGACCGTGGACGACATCATGCC
AGCCGTGAAAACCGTGATACGGTCTATCCGGATCCTGAAGTTCCTGG
TGGCCAAGCGGAAGTTCAAAGAGACACTGCGGCCCTACGACGTGAAG
GACGTGATCGAGCAGTATTCTGCCGGCCACCTGGACATGCTGGGCAG
AATCAAGAGCCTGCAGACCAGAGTGGACCAGATCGTTGGAAGAGGCC
CAGGCGACAGAAAGGCCAGAGAGAAGGGCGATAAGGGCCCATCTGAT
GCCGAGGTTGTCGACGAGATATCAATGATGGGCAGAGTGGTCAAGGT
GGAAAAACAGGTGCAGAGCATCGAGCACAAGCTGGACCTGCTGCTGG
GATTCTACAGCCGGTGTCTGAGAAGCGGCACATCTGCATCTCTGGGC
GCTGTGCAGGTCCCACTGTTCGACCCTGATATCACCAGCGACTATCA
CAGCCCCGTGGACCACGAGGACATCTCCGTTTCTGCTCAGACCCTGA
GCATCAGCAGATCCGTGTCCACCAACATGGACTAATAAGAGCTCGCT
GATCAGCCTCGACTGTGCCTTCTAGTTGCCAGCCATCTGTTGTTTGC
CCCTCCCCCGTGCCTTCCTTGACCCTGGAAGGTGCCACTCCCACTGT
CCTTTCCTAATAAAATGAGGAAATTGCATCGCATTGTCTGAGTAGGT
GTCATTCTATTCTGGGGGGTGGGGTGGGGCAGGACAGCAAGGGGGAG
GATTGGGAAGACAATAGCAGGCATGCTGGGGATGCGGTGGGCTCTAT
GGTTAATTAAGGTCGGGCAGGAAGAGGGCCTATTTCCCATGATTCCT
TCATATTTGCATATACGATACAAGGCTGTTAGAGAGATAATTAGAAT
TAATTTGACTGTAAACACAAAGATATTAGTACAAAATACGTGACGTA
GAAAGTAATAATTTCTTGGGTAGTTTGCAGTTTTAAAATTATGTTTT
AAAATGGACTATCATATGCTTACCGTAACTTGAAAGTATTTCGATTT
CTTGGCTTTATATATCTTGTGGAAAGGACGAAACACCGAGGAGCACC
AGGAACTTGCCAGTTTAAGTACTCTGTGCTGGAAACAGCACAGAATC
TACTTAAACAAGGCAAAATGCCGTGTTTATCTCGTCAACTTGTTGGC
GAGATTTTTTCTAAAGCTTGAATTCAAGCTTGAATTCAGCTGACGTG
CCTCGGACCGCTAGGAACCCCTAGTGATGGAGTTGGCCACTCCCTCT
CTGCGCGCTCGCTCGCTCACTGAGGCCGGGCGACCAAAGGTCGCCCG
ACGCCCGGGCTTTGCCCGGGCGGCCTCAGTGAGCGAGCGAGCGCGCA
G
Exemplary pITR-CAG.hKCNQ4codop.U6-mmu408Rev
sequence
(SEQ ID NO: 275)
CTGCGCGCTCGCTCGCTCACTGAGGCCGCCCGGGCGTCGGGCGACCT
TTGGTCGCCCGGCCTCAGTGAGCGAGCGAGCGCGCAGAGAGGGAGTG
GCCAACTCCATCACTAGGGGTTCCTGCGGCCGCACGCGTGACATTGA
TTATTGACTAGTTATTAATAGTAATCAATTACGGGGTCATTAGTTCA
TAGCCCATATATGGAGTTCCGCGTTACATAACTTACGGTAAATGGCC
CGCCTGGCTGACCGCCCAACGACCCCCGCCCATTGACGTCAATAATG
ACGTATGTTCCCATAGTAACGCCAATAGGGACTTTCCATTGACGTCA
ATGGGTGGACTATTTACGGTAAACTGCCCACTTGGCAGTACATCAAG
TGTATCATATGCCAAGTACGCCCCCTATTGACGTCAATGACGGTAAA
TGGCCCGCCTGGCATTATGCCCAGTACATGACCTTATGGGACTTTCC
TACTTGGCAGTACATCTACGTATTAGTCATCGCTATTACCATGGGTC
GAGGTGAGCCCCACGTTCTGCTTCACTCTCCCCATCTCCCCCCCCTC
CCCACCCCCAATTTTGTATTTATTTATTTTTTAATTATTTTGTGCAG
CGATGGGGGCGGGGGGGGGGGGGGCGCGCGCCAGGCGGGGCGGGGCG
GGGCGAGGGGCGGGGCGGGGCGAGGCGGAGAGGTGCGGCGGCAGCCA
ATCAGAGCGGCGCGCTCCGAAAGTTTCCTTTTATGGCGAGGCGGCGG
CGGCGGCGGCCCTATAAAAAGCGAAGCGCGCGGCGGGCGGGAGTCGC
TGCGTTGCCTTCGCCCCGTGCCCCGCTCCGCGCCGCCTCGCGCCGCC
CGCCCCGGCTCTGACTGACCGCGTTACTCCCACAGGTGAGCGGGCGG
GACGGCCCTTCTCCTCCGGGCTGTAATTAGCGCTTGGTTTAATGACG
GCTCGTTTCTTTTCTGTGGCTGCGTGAAAGCCTTAAAGGGCTCCGGG
AGGGCCCTTTGTGCGGGGGGGAGCGGCTCGGGGGGTGCGTGCGTGTG
TGTGTGCGTGGGGAGCGCCGCGTGCGGCCCGCGCTGCCCGGCGGCTG
TGAGCGCTGCGGGCGCGGCGCGGGGCTTTGTGCGCTCCGCGTGTGCG
CGAGGGGAGCGCGGCCGGGGGCGGTGCCCCGCGGTGCGGGGGGGCTG
CGAGGGGAACAAAGGCTGCGTGCGGGGTGTGTGCGTGGGGGGGTGAG
CAGGGGGTGTGGGCGCGGCGGTCGGGCTGTAACCCCCCCCTGCACCC
CCCTCCCCGAGTTGCTGAGCACGGCCCGGCTTCGGGTGCGGGGCTCC
GTGCGGGGCGTGGCGCGGGGCTCGCCGTGCCGGGCGGGGGGTGGCGG
CAGGTGGGGGTGCCGGGCGGGGCGGGGCCGCCTCGGGCCGGGGAGGG
CTCGGGGGAGGGGCGCGGCGGCCCCCGGAGCGCCGGCGGCTGTCGAG
GCGCGGCGAGCCGCAGCCATTGCCTTTTATGGTAATCGTGCGAGAGG
GCGCAGGGACTTCCTTTGTCCCAAATCTGTGCGGAGCCGAAATCTGG
GAGGCGCCGCCGCACCCCCTCTAGCGGGCGCGGGGCGAAGCGGTGCG
GCGCCGGCAGGAAGGAAATGGGCGGGGAGGGCCTTCGTGCGTCGCCG
CGCCGCCGTCCCCTTCTCCCTCTCCAGCCTCGGGGCTGTCCGCGGGG
GGACGGCTGCCTTCGGGGGGGACGGGGCAGGGCGGGGTTCGGCTTCT
GGCGTGTGACCGGCGGCTCTAGAGCCTCTGCTAACCATGTTCATGCC
TTCTTCTTTTTCCTACAGCTCCTGGGCAACGTGCTGGTTATTGTGAC
CGGTCGCTAGCCACCATGGCTGAAGCCCCTCCTAGAAGGCTTGGACT
GGGACCTCCTCCTGGGGATGCTCCTAGAGCTGAACTGGTGGCTCTGA
CAGCCGTGCAGTCTGAACAAGGCGAAGCTGGTGGCGGCGGATCTCCA
CGTAGACTTGGACTGCTGGGAAGCCCTCTTCCTCCTGGTGCTCCACT
TCCTGGACCTGGCAGTGGATCTGGATCTGCCTGTGGCCAGAGAAGCT
CTGCCGCTCACAAGAGATACCGGCGGCTGCAGAACTGGGTGTACAAC
GTGCTGGAAAGACCCAGAGGCTGGGCCTTCGTGTACCACGTGTTCAT
CTTTCTGCTGGTGTTCAGCTGCCTGGTGCTGTCCGTGCTGAGCACCA
TCCAAGAACATCAAGAGCTGGCTAACGAGTGCCTGTTAATACTGGAG
TTTGTGATGATTGTGGTGTTCGGCCTCGAGTACATCGTCCGCGTTTG
GAGCGCCGGCTGCTGCTGCAGATATAGAGGTTGGCAAGGCAGATTCC
GCTTCGCCAGAAAGCCCTTCTGCGTGATCGACTTCATCGTGTTCGTG
GCCAGCGTGGCCGTGATTGCTGCTGGCACACAGGGCAACATCTTCGC
CACAAGCGCCCTGCGGAGCATGCGGTTTCTGCAGATCCTGAGAATGG
TCCGAATGGACAGAAGAGGCGGCACCTGGAAGCTGCTGGGCTCTGTG
GTGTACGCCCACAGCAAAGAGCTGATCACCGCCTGGTACATCGGATT
TCTGGTGCTGATCTTCGCCTCCTTCCTGGTGTACCTGGCCGAGAAGG
ACGCCAACAGCGACTTTAGCAGCTACGCCGACTCTCTTTGGTGGGGC
ACCATCACACTGACCACCATCGGCTACGGCGACAAGACCCCTCACAC
ATGGCTGGGAAGAGTGCTGGCCGCTGGATTTGCTCTGCTGGGCATCA
GCTTTTTCGCCCTGCCTGCCGGAATCCTCGGATCTGGCTTTGCCCTG
AAGGTGCAAGAGCAGCACCGGCAGAAGCACTTCGAGAAGAGAAGAAT
GCCTGCCGCCAACCTGATTCAGGCCGCTTGGAGACTGTACAGCACCG
ACATGAGCAGAGCCTACCTGACCGCCACGTGGTATTATTACGACTCG
ATCCTGCCTAGCTTCCGCGAACTGGCCCTGCTGTTTGAGCATGTGCA
GAGAGCCAGAAACGGCGGCCTCAGACCTCTGGAAGTTCGGAGAGCAC
CTGTGCCTGATGGCGCCCCTTCTAGATATCCTCCAGTGGCCACCTGT
CACAGACCCGGCAGCACATCTTTTTGCCCTGGCGAGTCTAGCCGGAT
GGGCATCAAGGACAGAATCAGAATGGGCAGCAGCCAGCGGAGAACAG
GCCCTTCTAAACAGCATCTGGCCCCTCCAACCATGCCTACAAGCCCT
AGCTCTGAGCAAGTGGGCGAAGCCACCTCTCCTACCAAGGTGCAGAA
GTCCTGGTCCTTCAACGACCGGACCAGATTCAGAGCCAGCCTGAGAC
TGAAGCCCAGAACCTCTGCCGAGGATGCCCCTTCTGAAGAGGTGGCC
GAAGAGAAGTCCTACCAGTGCGAGCTGACCGTGGACGACATCATGCC
AGCCGTGAAAACCGTGATACGGTCTATCCGGATCCTGAAGTTCCTGG
TGGCCAAGCGGAAGTTCAAAGAGACACTGCGGCCCTACGACGTGAAG
GACGTGATCGAGCAGTATTCTGCCGGCCACCTGGACATGCTGGGCAG
AATCAAGAGCCTGCAGACCAGAGTGGACCAGATCGTTGGAAGAGGCC
CAGGCGACAGAAAGGCCAGAGAGAAGGGCGATAAGGGCCCATCTGAT
GCCGAGGTTGTCGACGAGATATCAATGATGGGCAGAGTGGTCAAGGT
GGAAAAACAGGTGCAGAGCATCGAGCACAAGCTGGACCTGCTGCTGG
GATTCTACAGCCGGTGTCTGAGAAGCGGCACATCTGCATCTCTGGGC
GCTGTGCAGGTCCCACTGTTCGACCCTGATATCACCAGCGACTATCA
CAGCCCCGTGGACCACGAGGACATCTCCGTTTCTGCTCAGACCCTGA
GCATCAGCAGATCCGTGTCCACCAACATGGACTAATAAGAGCTCGCT
GATCAGCCTCGACTGTGCCTTCTAGTTGCCAGCCATCTGTTGTTTGC
CCCTCCCCCGTGCCTTCCTTGACCCTGGAAGGTGCCACTCCCACTGT
CCTTTCCTAATAAAATGAGGAAATTGCATCGCATTGTCTGAGTAGGT
GTCATTCTATTCTGGGGGGTGGGGTGGGGCAGGACAGCAAGGGGGAG
GATTGGGAAGACAATAGCAGGCATGCTGGGGATGCGGTGGGCTCTAT
GGTTAATTAAGGTCGGGCAGGAAGAGGGCCTATTTCCCATGATTCCT
TCATATTTGCATATACGATACAAGGCTGTTAGAGAGATAATTAGAAT
TAATTTGACTGTAAACACAAAGATATTAGTACAAAATACGTGACGTA
GAAAGTAATAATTTCTTGGGTAGTTTGCAGTTTTAAAATTATGTTTT
AAAATGGACTATCATATGCTTACCGTAACTTGAAAGTATTTCGATTT
CTTGGCTTTATATATCTTGTGGAAAGGACGAAACACCGAAGCCAAAG
ACCACAATCATCGTTTAAGTACTCTGTGCTGGAAACAGCACAGAATC
TACTTAAACAAGGCAAAATGCCGTGTTTATCTCGTCAACTTGTTGGC
GAGATTTTTTCTAAAGCTTGAATTCAAGCTTGAATTCAGCTGACGTG
CCTCGGACCGCTAGGAACCCCTAGTGATGGAGTTGGCCACTCCCTCT
CTGCGCGCTCGCTCGCTCACTGAGGCCGGGCGACCAAAGGTCGCCCG
ACGCCCGGGCTTTGCCCGGGCGGCCTCAGTGAGCGAGCGAGCGCGCA
G

Exemplary Base Cas9 Plasmids

Exemplary pITR.CMVEnhProm-SaCas9_CRISPRi
sequence
(SEQ ID NO: 276)
CTGCGCGCTCGCTCGCTCACTGAGGCCGCCCGGGCGTCGGGCGACCT
TTGGTCGCCCGGCCTCAGTGAGCGAGCGAGCGCGCAGAGAGGGAGTG
GCCAACTCCATCACTAGGGGTTCCTGCGGCCGCACGCGTCGTTACAT
AACTTACGGTAAATGGCCCGCCTGGCTGACCGCCCAACGACCCCCGC
CCATTGAGGTCAATAATGAGGTATGTTCCCATAGTAACGCCAATAGG
GAGTTTCCATTGAGGTCAATGGGTGGAGTATTTACGGTAAACTGCCC
ACTTGGCAGTACATCAAGTGTATCATATGCCAAGTACGCCCCCTATT
GACGTCAATGACGGTAAATGGCCCGCCTGGCATTATGCCCAGTACAT
GACCTTATGGGACTTTCCTACTTGGCAGTACATCTACGTATTAGTCA
TCGCTATTACCATGGTGATGCGGTTTTGGCAGTACATCAATGGGCGT
GGATAGCGGTTTGACTCACGGGGATTTCCAAGTCTCCACCCCATTGA
CGTCAATGGGAGTTTGTTTTGGCACCAAAATCAACGGGACTTTCCAA
AATGTCGTAACAACTCCGCCCCATTGACGCAAATGGGCGGTAGGCGT
GTACGGTGGGAGGTCTATATAAGCAGAGCTCGTTTAGTGAACCGTca
gatcgcctggagacgcACCGGTGCCACCATGGCCCCTAAGAAGAAGC
GGAAAGTCGGCAGCGGCAAGCGGAACTATATCCTGGGCCTCGCCATC
GGCATCACCAGCGTTGGCTACGGCATCATCGACTACGAGACACGGGA
CGTGATCGATGCCGGCGTGCGGCTGTTTAAAGAGGCCAACGTCGAGA
ACAACGAGGGCCGCAGATCTAAGAGAGGCGCCAGACGGCTGAAGCGG
AGAAGAAGGCACAGAATCCAGAGAGTGAAGAAGCTGCTGTTCGACTA
CAACCTGCTGACCGACCACAGCGAGCTGAGCGGCATCAATCCTTACG
AGGCCAGAGTGAAGGGCCTGAGCCAGAAGCTGAGCGAGGAAGAGTTT
AGCGCCGCTCTGCTGCACCTGGCCAAGAGAAGAGGCGTGCACAACGT
GAACGAGGTGGAAGAGGACACCGGCAACGAGCTGTCCACCAAAGAGC
AGATCAGCCGGAACAGCAAGGCCCTGGAAGAGAAATACGTGGCCGAA
CTGCAGCTGGAACGGCTGAAAAAGGATGGCGAAGTGCGGGGCAGCAT
CAATCGGTTCAAGACCAGCGACTACGTGAAAGAAGCCAAACAGCTGC
TGAAGGTGCAGAAGGCCTACCACCAGCTGGACCAGAGCTTCATCGAC
ACCTACATCGACCTGCTGGAAACCCGGCGGACCTACTATGAAGGACC
TGGCGAGGGAAGCCCCTTCGGCTGGAAGGACATCAAAGAATGGTACG
AGATGCTGATGGGCCACTGCACCTACTTTCCCGAGGAACTGCGGAGC
GTGAAGTACGCCTACAACGCCGACCTGTACAACGCCCTGAACGACCT
GAACAACCTCGTGATCACCCGGGACGAGAACGAGAAGCTGGAATATT
AGGAGAAGTTCCAGATCATCGAGAACGTGTTCAAGCAGAAGAAGAAA
CCCACGCTGAAGCAGATCGCCAAAGAGATCCTGGTCAACGAGGAAGA
TATCAAGGGCTACAGAGTGACCAGCACCGGCAAGCCCGAGTTCACCA
ACCTGAAGGTGTACCACGACATCAAGGATATCACAGCCCGGAAAGAG
ATTATTGAGAACGCCGAGCTGCTGGACCAAATCGCCAAGATCCTGAC
CATCTACCAGAGCAGCGAGGACATCCAAGAGGAACTGACCAATCTGA
ACTCCGAGCTGACCCAAGAAGAGATCGAGCAGATCTCTAATCTGAAG
GGGTACACCGGCACACACAACCTGAGCCTGAAGGCCATCAACCTGAT
CCTGGACGAGCTGTGGCACACCAACGACAACCAGATTGCCATCTTCA
ACAGGCTGAAGCTGGTGCCCAAGAAGGTGGACCTGTCTCAGCAGAAA
GAAATCCCCACCACACTGGTGGACGACTTCATTCTGAGCCCCGTGGT
CAAGAGGAGCTTCATCCAGAGCATCAAAGTGATCAACGCCATCATCA
AGAAGTACGGGCTGCCCAACGATATCATCATCGAGCTGGCCCGCGAG
AAGAACTCCAAGGACGCCCAGAAAATGATCAACGAGATGCAGAAGAG
GAACCGCCAGACCAACGAGCGGATCGAGGAAATCATCCGGACCACCG
GCAAAGAGAACGCCAAGTACCTGATCGAGAAGATCAAGCTGCACGAC
ATGCAAGAGGGCAAGTGCCTGTACAGCCTGGAAGCCATTCCTCTGGA
AGATCTGCTGAACAATCCCTTCAACTATGAGGTGGACCACATCATCC
CCAGAAGCGTGTCCTTCGACAACAGCTTCAACAACAAGGTGCTCGTG
AAGCAAGAGGAAGCCAGCAAGAAGGGCAACAGAACCCCATTCCAGTA
CCTGAGCAGCAGCGACAGCAAGATCAGCTACGAAACCTTCAAGAAGC
ACATCCTGAATCTGGCCAAAGGCAAGGGCAGAATCAGCAAGACCAAG
AAAGAATACCTGCTCGAGGAACGGGACATCAACAGATTCAGCGTGCA
GAAAGACTTCATCAATCGGAACCTGGTGGACACCAGATACGCCACCA
GAGGCCTGATGAATCTGCTGCGGAGCTACTTCAGAGTGAACAATCTG
GACGTGAAAGTCAAGTCCATCAACGGCGGCTTCACCAGCTTTCTGCG
GCGGAAGTGGAAGTTCAAGAAAGAGCGGAACAAGGGCTATAAGCACC
ACGCCGAGGACGCCCTGATCATTGCCAACGCCGATTTCATCTTCAAA
GAGTGGAAGAAACTGGACAAGGCCAAAAAAGTGATGGAAAACCAGAT
GTTCGAGGAAAAGCAGGCCGAGAGCATGCCCGAGATCGAAACCGAGC
AAGAGTACAAAGAGATTTTCATCACGCCCCACCAGATCAAGCACATT
AAGGACTTCAAGGACTACAAGTACAGCCACCGCGTGGACAAGAAGCC
CAATAGAGAGCTGATTAACGACACCCTGTACTCTACCCGGAAGGACG
ACAAGGGCAATACCCTGATCGTCAACAACCTGAACGGCCTGTACGAC
AAGGACAACGACAAGCTCAAGAAGCTGATCAACAAGAGCCCCGAGAA
ACTGCTGATGTACCACCACGATCCTCAGACCTACCAGAAACTGAAGC
TCATCATGGAACAGTACGGCGACGAGAAGAATCCCCTGTAGAAGTAG
TAGGAGGAAACCGGGAACTACCTGACCAAGTACTCCAAAAAGGACAA
TGGGCCCGTGATCAAGAAGATTAAGTATTACGGCAACAAGCTGAATG
CCCACCTGGACATCACCGACGACTACCCCAACAGCAGAAACAAGGTG
GTCAAGCTGTCCCTGAAGCCTTACAGATTCGACGTGTACCTGGACAA
CGGCGTGTACAAGTTCGTGACCGTGAAGAACCTGGACGTCATCAAAA
AAGAAAACTAGTAGGAAGTCAACAGCAAGTGCTATGAGGAAGCCAAG
AAACTCAAGAAAATCAGCAACCAGGCCGAGTTTATCGCCTCCTTCTA
CAACAACGATCTGATCAAGATCAACGGGGAGCTGTATAGAGTGATCG
GCGTGAACAATGACCTGCTGAACCGGATCGAAGTGAACATGATCGAC
ATCACCTACCGCGAGTACCTCGAGAACATGAACGATAAGCGGCCTCC
ACGGATCATTAAGACAATCGCCAGCAAGACGCAGAGCATTAAGAAGT
ACAGCACAGACATCCTGGGCAACCTGTACGAAGTGAAGTCTAAGAAG
CACCCGCAGATTATCAAGAAAGGCAGCGGCGCTCCCAAGAAAAAACG
GAAGGTTTAAGAGCTCGCTGATCAGCCTCGATAAGATACATTGATGA
GTTTGGACAAACCACAACTAGAATGCAGTGAAAAAAATGCTTTATTT
GTGAAATTTGTGATGCTATTGCTTTATTTGTAACCATTATAAGCTGC
AATAAACAAGTTAAGCTTGAATTCAGCTGACGTGCCTCGGACCGCTA
GGAACCCCTAGTGATGGAGTTGGCCACTCCCTCTCTGCGCGCTCGCT
CGCTCACTGAGGCCGGGCGACCAAAGGTCGCCCGACGCCCGGGCTTT
GCCCGGGCGGCCTCAGTGAGCGAGCGAGCGCGCAG
Exemplary pITR.CMV-SaCas9_CRISPRi
sequence
(SEQ ID NO: 277)
CTGCGCGCTCGCTCGCTCACTGAGGCCGCCCGGGCGTCGGGCGACCT
TTGGTCGCCCGGCCTCAGTGAGCGAGCGAGCGCGCAGAGAGGGAGTG
GCCAACTCCATCACTAGGGGTTCCTGCGGCCGCACGCGTGTGATGCG
GTTTTGGCAGTACATCAATGGGCGTGGATAGCGGTTTGACTCACGGG
GATTTCCAAGTCTCCACCCCATTGACGTCAATGGGAGTTTGTTTTGG
CACCAAAATCAACGGGACTTTCCAAAATGTCGTAACAACTCCGCCCC
ATTGACGCAAATGGGCGGTAGGCGTGTACGGTGGGAGGTCTATATAA
GCAGAGCTCGTTTAGTGAACCGTcagatcgcctggagacgcACCGGT
GCCACCATGGCCCCTAAGAAGAAGCGGAAAGTCGGCAGCGGCAAGCG
GAACTATATCCTGGGCCTCGCCATCGGCATCACCAGCGTTGGCTACG
GCATCATCGACTACGAGACACGGGACGTGATCGATGCCGGCGTGCGG
CTGTTTAAAGAGGCCAACGTCGAGAACAACGAGGGCCGCAGATCTAA
GAGAGGCGCCAGACGGCTGAAGCGGAGAAGAAGGCACAGAATCCAGA
GAGTGAAGAAGCTGCTGTTCGACTACAACCTGCTGACCGACCACAGC
GAGCTGAGCGGCATCAATCCTTACGAGGCCAGAGTGAAGGGCCTGAG
CCAGAAGCTGAGCGAGGAAGAGTTTAGCGCCGCTCTGCTGCACCTGG
CCAAGAGAAGAGGCGTGCACAACGTGAACGAGGTGGAAGAGGACACC
GGCAACGAGCTGTCCACCAAAGAGCAGATCAGCCGGAACAGCAAGGC
CCTGGAAGAGAAATACGTGGCCGAACTGCAGCTGGAACGGCTGAAAA
AGGATGGCGAAGTGCGGGGCAGCATCAATCGGTTCAAGACCAGCGAC
TACGTGAAAGAAGCCAAACAGCTGCTGAAGGTGCAGAAGGCCTACCA
CCAGCTGGACCAGAGCTTCATCGACACCTACATCGACCTGCTGGAAA
CCCGGCGGACCTACTATGAAGGACCTGGCGAGGGAAGCCCCTTCGGC
TGGAAGGACATCAAAGAATGGTACGAGATGCTGATGGGCCACTGCAC
CTACTTTCCCGAGGAACTGCGGAGCGTGAAGTACGCCTACAACGCCG
ACCTGTACAACGCCCTGAACGACCTGAACAACCTCGTGATCACCCGG
GACGAGAACGAGAAGCTGGAATATTAGGAGAAGTTCCAGATCATCGA
GAACGTGTTCAAGCAGAAGAAGAAACCCACGCTGAAGCAGATCGCCA
AAGAGATCCTGGTCAACGAGGAAGATATCAAGGGCTACAGAGTGACC
AGCACCGGCAAGCCCGAGTTCACCAACCTGAAGGTGTACCACGACAT
CAAGGATATCACAGCCCGGAAAGAGATTATTGAGAACGCCGAGCTGC
TGGACCAAATCGCCAAGATCCTGACCATCTACCAGAGCAGCGAGGAC
ATCCAAGAGGAACTGACCAATCTGAACTCCGAGCTGACCCAAGAAGA
GATCGAGCAGATCTCTAATCTGAAGGGGTACACCGGCACACACAACC
TGAGCCTGAAGGCCATCAACCTGATCCTGGACGAGCTGTGGCACACC
AACGACAACCAGATTGCCATCTTCAACAGGCTGAAGCTGGTGCCCAA
GAAGGTGGACCTGTCTCAGCAGAAAGAAATCCCCACCACACTGGTGG
ACGACTTCATTCTGAGCCCCGTGGTCAAGAGGAGCTTCATCCAGAGC
ATCAAAGTGATCAACGCCATCATCAAGAAGTACGGGCTGCCCAACGA
TATCATCATCGAGCTGGCCCGCGAGAAGAACTCCAAGGACGCCCAGA
AAATGATCAACGAGATGCAGAAGAGGAACCGCCAGACCAACGAGCGG
ATCGAGGAAATCATCCGGACCACCGGCAAAGAGAACGCCAAGTACCT
GATCGAGAAGATCAAGCTGCACGACATGCAAGAGGGCAAGTGCCTGT
ACAGCCTGGAAGCCATTCCTCTGGAAGATCTGCTGAACAATCCCTTC
AACTATGAGGTGGACCACATCATCCCCAGAAGCGTGTCCTTCGACAA
CAGCTTCAACAACAAGGTGCTCGTGAAGCAAGAGGAAGCCAGCAAGA
AGGGCAACAGAACCCCATTCCAGTACCTGAGCAGCAGCGACAGCAAG
ATCAGCTACGAAACCTTCAAGAAGCACATCCTGAATCTGGCCAAAGG
CAAGGGCAGAATCAGCAAGACCAAGAAAGAATACCTGCTCGAGGAAC
GGGACATCAACAGATTCAGCGTGCAGAAAGACTTCATCAATCGGAAC
CTGGTGGACACCAGATACGCCACCAGAGGCCTGATGAATCTGCTGCG
GAGCTACTTCAGAGTGAACAATCTGGACGTGAAAGTCAAGTCCATCA
ACGGCGGCTTCACCAGCTTTCTGCGGCGGAAGTGGAAGTTCAAGAAA
GAGCGGAACAAGGGCTATAAGCACCACGCCGAGGACGCCCTGATCAT
TGCCAACGCCGATTTCATCTTCAAAGAGTGGAAGAAACTGGACAAGG
CCAAAAAAGTGATGGAAAACCAGATGTTCGAGGAAAAGCAGGCCGAG
AGCATGCCCGAGATCGAAACCGAGCAAGAGTACAAAGAGATTTTCAT
CACGCCCCACCAGATCAAGCACATTAAGGACTTCAAGGACTACAAGT
ACAGCCACCGCGTGGACAAGAAGCCCAATAGAGAGCTGATTAACGAC
ACCCTGTACTCTACCCGGAAGGACGACAAGGGCAATACCCTGATCGT
CAACAACCTGAACGGCCTGTACGACAAGGACAACGACAAGCTCAAGA
AGCTGATCAACAAGAGCCCCGAGAAACTGCTGATGTACCACCACGAT
CCTCAGACCTACCAGAAACTGAAGCTCATCATGGAACAGTACGGCGA
CGAGAAGAATCCCCTGTACAAGTACTACGAGGAAACCGGGAACTACC
TGACCAAGTACTCCAAAAAGGACAATGGGCCCGTGATCAAGAAGATT
AAGTATTACGGCAACAAGCTGAATGCCCACCTGGACATCACCGACGA
CTACCCCAACAGCAGAAACAAGGTGGTCAAGCTGTCCCTGAAGCCTT
ACAGATTCGACGTGTACCTGGACAACGGCGTGTACAAGTTCGTGACC
GTGAAGAACCTGGACGTCATCAAAAAAGAAAACTACTACGAAGTCAA
CAGCAAGTGCTATGAGGAAGCCAAGAAACTCAAGAAAATCAGCAACC
AGGCCGAGTTTATCGCCTCCTTCTACAACAACGATCTGATCAAGATC
AACGGGGAGCTGTATAGAGTGATCGGCGTGAACAATGACCTGCTGAA
CCGGATCGAAGTGAACATGATCGACATCACCTACCGCGAGTACCTCG
AGAACATGAACGATAAGCGGCCTCCACGGATCATTAAGACAATCGCC
AGCAAGACGCAGAGCATTAAGAAGTACAGCACAGACATCCTGGGCAA
CCTGTACGAAGTGAAGTCTAAGAAGCACCCGCAGATTATCAAGAAAG
GCAGCGGCGCTCCCAAGAAAAAACGGAAGGTTTAAGAGCTCGCTGAT
CAGCCTCGATAAGATACATTGATGAGTTTGGACAAACCACAACTAGA
ATGCAGTGAAAAAAATGCTTTATTTGTGAAATTTGTGATGCTATTGC
TTTATTTGTAACCATTATAAGCTGCAATAAACAAGTTAAGCTTGAAT
TCAGCTGACGTGCCTCGGACCGCTAGGAACCCCTAGTGATGGAGTTG
GCCACTCCCTCTCTGCGCGCTCGCTCGCTCACTGAGGCCGGGCGACC
AAAGGTCGCCCGACGCCCGGGCTTTGCCCGGGCGGCCTCAGTGAGCG
AGCGAGCGCGCAG
Exemplary pITR.CMVEnhProm.SaCas9
sequence
(SEQ ID NO: 278)
CTGCGCGCTCGCTCGCTCACTGAGGCCGCCCGGGCGTCGGGCGACCT
TTGGTCGCCCGGCCTCAGTGAGCGAGCGAGCGCGCAGAGAGGGAGTG
GCCAACTCCATCACTAGGGGTTCCTGCGGCCGCACGCGTCGTTACAT
AACTTACGGTAAATGGCCCGCCTGGCTGACCGCCCAACGACCCCCGC
CCATTGACGTCAATAATGACGTATGTTCCCATAGTAACGCCAATAGG
GACTTTCCATTGACGTCAATGGGTGGAGTATTTACGGTAAACTGCCC
ACTTGGCAGTACATCAAGTGTATCATATGCCAAGTACGCCCCCTATT
GACGTCAATGACGGTAAATGGCCCGCCTGGCATTATGCCCAGTACAT
GACCTTATGGGACTTTCCTACTTGGCAGTACATCTACGTATTAGTCA
TCGCTATTACCATGGTGATGCGGTTTTGGCAGTACATCAATGGGCGT
GGATAGCGGTTTGACTCACGGGGATTTCCAAGTCTCCACCCCATTGA
CGTCAATGGGAGTTTGTTTTGGCACCAAAATCAACGGGACTTTCCAA
AATGTCGTAACAACTCCGCCCCATTGACGCAAATGGGCGGTAGGCGT
GTACGGTGGGAGGTCTATATAAGCAGAGCTCGTTTAGTGAACCGTca
gatcgcctggagacgcACCGGTGCCACCATGGCCCCTAAGAAGAAGC
GGAAAGTCGGCAGCGGCAAGCGGAACTATATCCTGGGCCTCGACATC
GGCATCACCAGCGTTGGCTACGGCATCATCGACTACGAGACACGGGA
CGTGATCGATGCCGGCGTGCGGCTGTTTAAAGAGGCCAACGTCGAGA
ACAACGAGGGCCGCAGATCTAAGAGAGGCGCCAGACGGCTGAAGCGG
AGAAGAAGGCACAGAATCCAGAGAGTGAAGAAGCTGCTGTTCGACTA
CAACCTGCTGACCGACCACAGCGAGCTGAGCGGCATCAATCCTTACG
AGGCCAGAGTGAAGGGCCTGAGCCAGAAGCTGAGCGAGGAAGAGTTT
AGCGCCGCTCTGCTGCACCTGGCCAAGAGAAGAGGCGTGCACAACGT
GAACGAGGTGGAAGAGGACACCGGCAACGAGCTGTCCACCAAAGAGC
AGATCAGCCGGAACAGCAAGGCCCTGGAAGAGAAATACGTGGCCGAA
CTGCAGCTGGAACGGCTGAAAAAGGATGGCGAAGTGCGGGGCAGCAT
CAATCGGTTCAAGACCAGCGACTACGTGAAAGAAGCCAAACAGCTGC
TGAAGGTGCAGAAGGCCTACCACCAGCTGGACCAGAGCTTCATCGAC
ACCTACATCGACCTGCTGGAAACCCGGCGGACCTACTATGAAGGACC
TGGCGAGGGAAGCCCCTTCGGCTGGAAGGACATCAAAGAATGGTACG
AGATGCTGATGGGCCACTGCACCTACTTTCCCGAGGAACTGCGGAGC
GTGAAGTACGCCTACAACGCCGACCTGTACAACGCCCTGAACGACCT
GAACAACCTCGTGATCACCCGGGACGAGAACGAGAAGCTGGAATATT
AGGAGAAGTTCCAGATCATCGAGAACGTGTTCAAGCAGAAGAAGAAA
CCCACGCTGAAGCAGATCGCCAAAGAGATCCTGGTCAACGAGGAAGA
TATCAAGGGCTACAGAGTGACCAGCACCGGCAAGCCCGAGTTCACCA
ACCTGAAGGTGTACCACGACATCAAGGATATCACAGCCCGGAAAGAG
ATTATTGAGAACGCCGAGCTGCTGGACCAAATCGCCAAGATCCTGAC
CATCTACCAGAGCAGCGAGGACATCCAAGAGGAACTGACCAATCTGA
ACTCCGAGCTGACCCAAGAAGAGATCGAGCAGATCTCTAATCTGAAG
GGGTACACCGGCACACACAACCTGAGCCTGAAGGCCATCAACCTGAT
CCTGGACGAGCTGTGGCACACCAACGACAACCAGATTGCCATCTTCA
ACAGGCTGAAGCTGGTGCCCAAGAAGGTGGACCTGTCTCAGCAGAAA
GAAATCCCCACCACACTGGTGGACGACTTCATTCTGAGCCCCGTGGT
CAAGAGGAGCTTCATCCAGAGCATCAAAGTGATCAACGCCATCATCA
AGAAGTACGGGCTGCCCAACGATATCATCATCGAGCTGGCCCGCGAG
AAGAACTCCAAGGACGCCCAGAAAATGATCAACGAGATGCAGAAGAG
GAACCGCCAGACCAACGAGCGGATCGAGGAAATCATCCGGACCACCG
GCAAAGAGAACGCCAAGTACCTGATCGAGAAGATCAAGCTGCACGAC
ATGCAAGAGGGCAAGTGCCTGTACAGCCTGGAAGCCATTCCTCTGGA
AGATCTGCTGAACAATCCCTTCAACTATGAGGTGGACCACATCATCC
CCAGAAGCGTGTCCTTCGACAACAGCTTCAACAACAAGGTGCTCGTG
AAGCAAGAGGAAAACAGCAAGAAGGGCAACAGAACCCCATTCCAGTA
CCTGAGCAGCAGCGACAGCAAGATCAGCTACGAAACCTTCAAGAAGC
ACATCCTGAATCTGGCCAAAGGCAAGGGCAGAATCAGCAAGACCAAG
AAAGAATACCTGCTCGAGGAACGGGACATCAACAGATTCAGCGTGCA
GAAAGACTTCATCAATCGGAACCTGGTGGACACCAGATACGCCACCA
GAGGCCTGATGAATCTGCTGCGGAGCTACTTCAGAGTGAACAATCTG
GACGTGAAAGTCAAGTCCATCAACGGCGGCTTCACCAGCTTTCTGCG
GCGGAAGTGGAAGTTCAAGAAAGAGCGGAACAAGGGCTATAAGCACC
ACGCCGAGGACGCCCTGATCATTGCCAACGCCGATTTCATCTTCAAA
GAGTGGAAGAAACTGGACAAGGCCAAAAAAGTGATGGAAAACCAGAT
GTTCGAGGAAAAGCAGGCCGAGAGCATGCCCGAGATCGAAACCGAGC
AAGAGTACAAAGAGATTTTCATCACGCCCCACCAGATCAAGCACATT
AAGGACTTCAAGGACTACAAGTACAGCCACCGCGTGGACAAGAAGCC
CAATAGAGAGCTGATTAACGACACCCTGTACTCTACCCGGAAGGACG
ACAAGGGCAATACCCTGATCGTCAACAACCTGAACGGCCTGTACGAC
AAGGACAACGACAAGCTCAAGAAGCTGATCAACAAGAGCCCCGAGAA
ACTGCTGATGTACCACCACGATCCTCAGACCTACCAGAAACTGAAGC
TCATCATGGAACAGTACGGCGACGAGAAGAATCCCCTGTAGAAGTAG
TAGGAGGAAACCGGGAACTACCTGACCAAGTACTCCAAAAAGGACAA
TGGGCCCGTGATCAAGAAGATTAAGTATTACGGCAACAAGCTGAATG
CCCACCTGGACATCACCGACGACTACCCCAACAGCAGAAACAAGGTG
GTCAAGCTGTCCCTGAAGCCTTACAGATTCGACGTGTACCTGGACAA
CGGCGTGTACAAGTTCGTGACCGTGAAGAACCTGGACGTCATCAAAA
AAGAAAACTAGTAGGAAGTCAACAGCAAGTGCTATGAGGAAGCCAAG
AAACTCAAGAAAATCAGCAACCAGGCCGAGTTTATCGCCTCCTTCTA
CAACAACGATCTGATCAAGATCAACGGGGAGCTGTATAGAGTGATCG
GCGTGAACAATGACCTGCTGAACCGGATCGAAGTGAACATGATCGAC
ATCACCTACCGCGAGTACCTCGAGAACATGAACGATAAGCGGCCTCC
ACGGATCATTAAGACAATCGCCAGCAAGACGCAGAGCATTAAGAAGT
ACAGCACAGACATCCTGGGCAACCTGTACGAAGTGAAGTCTAAGAAG
CACCCGCAGATTATCAAGAAAGGCAGCGGCGCTCCCAAGAAAAAACG
GAAGGTTTAAGAGCTCGCTGATCAGCCTCGATAAGATACATTGATGA
GTTTGGACAAACCACAACTAGAATGCAGTGAAAAAAATGCTTTATTT
GTGAAATTTGTGATGCTATTGCTTTATTTGTAACCATTATAAGCTGC
AATAAACAAGTTAAGCTTGAATTCAGCTGACGTGCCTCGGACCGCTA
GGAACCCCTAGTGATGGAGTTGGCCACTCCCTCTCTGCGCGCTCGCT
CGCTCACTGAGGCCGGGCGACCAAAGGTCGCCCGACGCCCGGGCTTT
GCCCGGGCGGCCTCAGTGAGCGAGCGAGCGCGCAG
Exemplary pITR.CMV.SaCas9
sequence
(SEQ ID NO: 279)
CTGCGCGCTCGCTCGCTCACTGAGGCCGCCCGGGCGTCGGGCGACCT
TTGGTCGCCCGGCCTCAGTGAGCGAGCGAGCGCGCAGAGAGGGAGTG
GCCAACTCCATCACTAGGGGTTCCTGCGGCCGCACGCGTGTGATGCG
GTTTTGGCAGTACATCAATGGGCGTGGATAGCGGTTTGACTCACGGG
GATTTCCAAGTCTCCACCCCATTGACGTCAATGGGAGTTTGTTTTGG
CACCAAAATCAACGGGACTTTCCAAAATGTCGTAACAACTCCGCCCC
ATTGACGCAAATGGGCGGTAGGCGTGTACGGTGGGAGGTCTATATAA
GCAGAGCTCGTTTAGTGAACCGTcagatcgcctggagacgcACCGGT
GCCACCATGGCCCCTAAGAAGAAGCGGAAAGTCGGCAGCGGCAAGCG
GAACTATATCCTGGGCCTCGACATCGGCATCACCAGCGTTGGCTACG
GCATCATCGACTACGAGACACGGGACGTGATCGATGCCGGCGTGCGG
CTGTTTAAAGAGGCCAACGTCGAGAACAACGAGGGCCGCAGATCTAA
GAGAGGCGCCAGACGGCTGAAGCGGAGAAGAAGGCACAGAATCCAGA
GAGTGAAGAAGCTGCTGTTCGACTACAACCTGCTGACCGACCACAGC
GAGCTGAGCGGCATCAATCCTTACGAGGCCAGAGTGAAGGGCCTGAG
CCAGAAGCTGAGCGAGGAAGAGTTTAGCGCCGCTCTGCTGCACCTGG
CCAAGAGAAGAGGCGTGCACAACGTGAACGAGGTGGAAGAGGACACC
GGCAACGAGCTGTCCACCAAAGAGCAGATCAGCCGGAACAGCAAGGC
CCTGGAAGAGAAATACGTGGCCGAACTGCAGCTGGAACGGCTGAAAA
AGGATGGCGAAGTGCGGGGCAGCATCAATCGGTTCAAGACCAGCGAC
TACGTGAAAGAAGCCAAACAGCTGCTGAAGGTGCAGAAGGCCTACCA
CCAGCTGGACCAGAGCTTCATCGACACCTACATCGACCTGCTGGAAA
CCCGGCGGACCTACTATGAAGGACCTGGCGAGGGAAGCCCCTTCGGC
TGGAAGGACATCAAAGAATGGTACGAGATGCTGATGGGCCACTGCAC
CTACTTTCCCGAGGAACTGCGGAGCGTGAAGTACGCCTACAACGCCG
ACCTGTACAACGCCCTGAACGACCTGAACAACCTCGTGATCACCCGG
GACGAGAACGAGAAGCTGGAATATTAGGAGAAGTTCCAGATCATCGA
GAACGTGTTCAAGCAGAAGAAGAAACCCACGCTGAAGCAGATCGCCA
AAGAGATCCTGGTCAACGAGGAAGATATCAAGGGCTACAGAGTGACC
AGCACCGGCAAGCCCGAGTTCACCAACCTGAAGGTGTACCACGACAT
CAAGGATATCACAGCCCGGAAAGAGATTATTGAGAACGCCGAGCTGC
TGGACCAAATCGCCAAGATCCTGACCATCTACCAGAGCAGCGAGGAC
ATCCAAGAGGAACTGACCAATCTGAACTCCGAGCTGACCCAAGAAGA
GATCGAGCAGATCTCTAATCTGAAGGGGTACACCGGCACACACAACC
TGAGCCTGAAGGCCATCAACCTGATCCTGGACGAGCTGTGGCACACC
AACGACAACCAGATTGCCATCTTCAACAGGCTGAAGCTGGTGCCCAA
GAAGGTGGACCTGTCTCAGCAGAAAGAAATCCCCACCACACTGGTGG
ACGACTTCATTCTGAGCCCCGTGGTCAAGAGGAGCTTCATCCAGAGC
ATCAAAGTGATCAACGCCATCATCAAGAAGTACGGGCTGCCCAACGA
TATCATCATCGAGCTGGCCCGCGAGAAGAACTCCAAGGACGCCCAGA
AAATGATCAACGAGATGCAGAAGAGGAACCGCCAGACCAACGAGCGG
ATCGAGGAAATCATCCGGACCACCGGCAAAGAGAACGCCAAGTACCT
GATCGAGAAGATCAAGCTGCACGACATGCAAGAGGGCAAGTGCCTGT
ACAGCCTGGAAGCCATTCCTCTGGAAGATCTGCTGAACAATCCCTTC
AACTATGAGGTGGACCACATCATCCCCAGAAGCGTGTCCTTCGACAA
CAGCTTCAACAACAAGGTGCTCGTGAAGCAAGAGGAAAACAGCAAGA
AGGGCAACAGAACCCCATTCCAGTACCTGAGCAGCAGCGACAGCAAG
ATCAGCTACGAAACCTTCAAGAAGCACATCCTGAATCTGGCCAAAGG
CAAGGGCAGAATCAGCAAGACCAAGAAAGAATACCTGCTCGAGGAAC
GGGACATCAACAGATTCAGCGTGCAGAAAGACTTCATCAATCGGAAC
CTGGTGGACACCAGATACGCCACCAGAGGCCTGATGAATCTGCTGCG
GAGCTACTTCAGAGTGAACAATCTGGACGTGAAAGTCAAGTCCATCA
ACGGCGGCTTCACCAGCTTTCTGCGGCGGAAGTGGAAGTTCAAGAAA
GAGCGGAACAAGGGCTATAAGCACCACGCCGAGGACGCCCTGATCAT
TGCCAACGCCGATTTCATCTTCAAAGAGTGGAAGAAACTGGACAAGG
CCAAAAAAGTGATGGAAAACCAGATGTTCGAGGAAAAGCAGGCCGAG
AGCATGCCCGAGATCGAAACCGAGCAAGAGTACAAAGAGATTTTCAT
CACGCCCCACCAGATCAAGCACATTAAGGACTTCAAGGACTACAAGT
ACAGCCACCGCGTGGACAAGAAGCCCAATAGAGAGCTGATTAACGAC
ACCCTGTACTCTACCCGGAAGGACGACAAGGGCAATACCCTGATCGT
CAACAACCTGAACGGCCTGTACGACAAGGACAACGACAAGCTCAAGA
AGCTGATCAACAAGAGCCCCGAGAAACTGCTGATGTACCACCACGAT
CCTCAGACCTACCAGAAACTGAAGCTCATCATGGAACAGTACGGCGA
CGAGAAGAATCCCCTGTACAAGTACTACGAGGAAACCGGGAACTACC
TGACCAAGTACTCCAAAAAGGACAATGGGCCCGTGATCAAGAAGATT
AAGTATTACGGCAACAAGCTGAATGCCCACCTGGACATCACCGACGA
CTACCCCAACAGCAGAAACAAGGTGGTCAAGCTGTCCCTGAAGCCTT
ACAGATTCGACGTGTACCTGGACAACGGCGTGTACAAGTTCGTGACC
GTGAAGAACCTGGACGTCATCAAAAAAGAAAACTACTACGAAGTCAA
CAGCAAGTGCTATGAGGAAGCCAAGAAACTCAAGAAAATCAGCAACC
AGGCCGAGTTTATCGCCTCCTTCTACAACAACGATCTGATCAAGATC
AACGGGGAGCTGTATAGAGTGATCGGCGTGAACAATGACCTGCTGAA
CCGGATCGAAGTGAACATGATCGACATCACCTACCGCGAGTACCTCG
AGAACATGAACGATAAGCGGCCTCCACGGATCATTAAGACAATCGCC
AGCAAGACGCAGAGCATTAAGAAGTACAGCACAGACATCCTGGGCAA
CCTGTACGAAGTGAAGTCTAAGAAGCACCCGCAGATTATCAAGAAAG
GCAGCGGCGCTCCCAAGAAAAAACGGAAGGTTTAAGAGCTCGCTGAT
CAGCCTCGATAAGATACATTGATGAGTTTGGACAAACCACAACTAGA
ATGCAGTGAAAAAAATGCTTTATTTGTGAAATTTGTGATGCTATTGC
TTTATTTGTAACCATTATAAGCTGCAATAAACAAGTTAAGCTTGAAT
TCAGCTGACGTGCCTCGGACCGCTAGGAACCCCTAGTGATGGAGTTG
GCCACTCCCTCTCTGCGCGCTCGCTCGCTCACTGAGGCCGGGCGACC
AAAGGTCGCCCGACGCCCGGGCTTTGCCCGGGCGGCCTCAGTGAGCG
AGCGAGCGCGCAG
Exemplary pITR.CMVEnhProm-SaCas9_CRISPRi
sequence
(SEQ ID NO: 280)
CCTGCAGGCAGCTGCGCGCTCGCTCGCTCACTGAGGCCGCCCGGGCG
TCGGGCGACCTTTGGTCGCCCGGCCTCAGTGAGCGAGCGAGCGCGCA
GAGAGGGAGTGGCCAACTCCATCACTAGGGGTTCCTGCGGCCGCACG
CGTCGTTACATAACTTACGGTAAATGGCCCGCCTGGCTGACCGCCCA
ACGACCCCCGCCCATTGACGTCAATAATGACGTATGTTCCCATAGTA
ACGCCAATAGGGACTTTCCATTGACGTCAATGGGTGGAGTATTTACG
GTAAACTGCCCACTTGGCAGTACATCAAGTGTATCATATGCCAAGTA
CGCCCCCTATTGACGTCAATGACGGTAAATGGCCCGCCTGGCATTAT
GCCCAGTACATGACCTTATGGGACTTTCCTACTTGGCAGTACATCTA
CGTATTAGTCATCGCTATTACCATGGTGATGCGGTTTTGGCAGTACA
TCAATGGGCGTGGATAGCGGTTTGACTCACGGGGATTTCCAAGTCTC
CACCCCATTGACGTCAATGGGAGTTTGTTTTGGCACCAAAATCAACG
GGACTTTCCAAAATGTCGTAACAACTCCGCCCCATTGACGCAAATGG
GCGGTAGGCGTGTACGGTGGGAGGTCTATATAAGCAGAGCTCGTTTA
GTGAACCGTcagatcgcctggagacgcACCGGTGCCACCATGGCCCC
TAAGAAGAAGCGGAAAGTCGGCAGCGGCAAGCGGAACTATATCCTGG
GCCTCGCCATCGGCATCACCAGCGTTGGCTACGGCATCATCGACTAC
GAGACACGGGACGTGATCGATGCCGGCGTGCGGCTGTTTAAAGAGGC
CAACGTCGAGAACAACGAGGGCCGCAGATCTAAGAGAGGCGCCAGAC
GGCTGAAGCGGAGAAGAAGGCACAGAATCCAGAGAGTGAAGAAGCTG
CTGTTCGACTACAACCTGCTGACCGACCACAGCGAGCTGAGCGGCAT
CAATCCTTACGAGGCCAGAGTGAAGGGCCTGAGCCAGAAGCTGAGCG
AGGAAGAGTTTAGCGCCGCTCTGCTGCACCTGGCCAAGAGAAGAGGC
GTGCACAACGTGAACGAGGTGGAAGAGGACACCGGCAACGAGCTGTC
CACCAAAGAGCAGATCAGCCGGAACAGCAAGGCCCTGGAAGAGAAAT
ACGTGGCCGAACTGCAGCTGGAACGGCTGAAAAAGGATGGCGAAGTG
CGGGGCAGCATCAATCGGTTCAAGACCAGCGACTACGTGAAAGAAGC
CAAACAGCTGCTGAAGGTGCAGAAGGCCTACCACCAGCTGGACCAGA
GCTTCATCGACACCTACATCGACCTGCTGGAAACCCGGCGGACCTAC
TATGAAGGACCTGGCGAGGGAAGCCCCTTCGGCTGGAAGGACATCAA
AGAATGGTACGAGATGCTGATGGGCCACTGCACCTACTTTCCCGAGG
AACTGCGGAGCGTGAAGTACGCCTACAACGCCGACCTGTACAACGCC
CTGAACGACCTGAACAACCTCGTGATCACCCGGGACGAGAACGAGAA
GCTGGAATATTAGGAGAAGTTCCAGATCATCGAGAACGTGTTCAAGC
AGAAGAAGAAACCCACGCTGAAGCAGATCGCCAAAGAGATCCTGGTC
AACGAGGAAGATATCAAGGGCTACAGAGTGACCAGCACCGGCAAGCC
CGAGTTCACCAACCTGAAGGTGTACCACGACATCAAGGATATCACAG
CCCGGAAAGAGATTATTGAGAACGCCGAGCTGCTGGACCAAATCGCC
AAGATCCTGACCATCTACCAGAGCAGCGAGGACATCCAAGAGGAACT
GACCAATCTGAACTCCGAGCTGACCCAAGAAGAGATCGAGCAGATCT
CTAATCTGAAGGGGTACACCGGCACACACAACCTGAGCCTGAAGGCC
ATCAACCTGATCCTGGACGAGCTGTGGCACACCAACGACAACCAGAT
TGCCATCTTCAACAGGCTGAAGCTGGTGCCCAAGAAGGTGGACCTGT
CTCAGCAGAAAGAAATCCCCACCACACTGGTGGACGACTTCATTCTG
AGCCCCGTGGTCAAGAGGAGCTTCATCCAGAGCATCAAAGTGATCAA
CGCCATCATCAAGAAGTACGGGCTGCCCAACGATATCATCATCGAGC
TGGCCCGCGAGAAGAACTCCAAGGACGCCCAGAAAATGATCAACGAG
ATGCAGAAGAGGAACCGCCAGACCAACGAGCGGATCGAGGAAATCAT
CCGGACCACCGGCAAAGAGAACGCCAAGTACCTGATCGAGAAGATCA
AGCTGCACGACATGCAAGAGGGCAAGTGCCTGTACAGCCTGGAAGCC
ATTCCTCTGGAAGATCTGCTGAACAATCCCTTCAACTATGAGGTGGA
CCACATCATCCCCAGAAGCGTGTCCTTCGACAACAGCTTCAACAACA
AGGTGCTCGTGAAGCAAGAGGAAGCCAGCAAGAAGGGCAACAGAACC
CCATTCCAGTACCTGAGCAGCAGCGACAGCAAGATCAGCTACGAAAC
CTTCAAGAAGCACATCCTGAATCTGGCCAAAGGCAAGGGCAGAATCA
GCAAGACCAAGAAAGAATACCTGCTCGAGGAACGGGACATCAACAGA
TTCAGCGTGCAGAAAGACTTCATCAATCGGAACCTGGTGGACACCAG
ATACGCCACCAGAGGCCTGATGAATCTGCTGCGGAGCTACTTCAGAG
TGAACAATCTGGACGTGAAAGTCAAGTCCATCAACGGCGGCTTCACC
AGCTTTCTGCGGCGGAAGTGGAAGTTCAAGAAAGAGCGGAACAAGGG
CTATAAGCACCACGCCGAGGACGCCCTGATCATTGCCAACGCCGATT
TCATCTTCAAAGAGTGGAAGAAACTGGACAAGGCCAAAAAAGTGATG
GAAAACCAGATGTTCGAGGAAAAGCAGGCCGAGAGCATGCCCGAGAT
CGAAACCGAGCAAGAGTACAAAGAGATTTTCATCACGCCCCACCAGA
TCAAGCACATTAAGGACTTCAAGGACTACAAGTACAGCCACCGCGTG
GACAAGAAGCCCAATAGAGAGCTGATTAACGACACCCTGTACTCTAC
CCGGAAGGACGACAAGGGCAATACCCTGATCGTCAACAACCTGAACG
GCCTGTACGACAAGGACAACGACAAGCTCAAGAAGCTGATCAACAAG
AGCCCCGAGAAACTGCTGATGTACCACCACGATCCTCAGACCTACCA
GAAACTGAAGCTCATCATGGAACAGTACGGCGACGAGAAGAATCCCC
TGTACAAGTACTACGAGGAAACCGGGAACTACCTGACCAAGTACTCC
AAAAAGGACAATGGGCCCGTGATCAAGAAGATTAAGTATTACGGCAA
CAAGCTGAATGCCCACCTGGACATCACCGACGACTACCCCAACAGCA
GAAACAAGGTGGTCAAGCTGTCCCTGAAGCCTTACAGATTCGACGTG
TACCTGGACAACGGCGTGTACAAGTTCGTGACCGTGAAGAACCTGGA
CGTCATCAAAAAAGAAAACTACTACGAAGTCAACAGCAAGTGCTATG
AGGAAGCCAAGAAACTCAAGAAAATGAGCAACGAGGCCGAGTTTATC
GCCTCCTTCTACAACAACGATCTGATCAAGATCAACGGGGAGCTGTA
TAGAGTGATCGGCGTGAACAATGACCTGCTGAACCGGATCGAAGTGA
ACATGATCGACATCACCTACCGCGAGTACCTCGAGAACATGAACGAT
AAGCGGCCTCCACGGATCATTAAGACAATCGCCAGCAAGACGCAGAG
CATTAAGAAGTACAGCACAGACATCCTGGGCAACCTGTACGAAGTGA
AGTCTAAGAAGCACCCGCAGATTATCAAGAAAGGCAGCGGCGCTCCC
AAGAAAAAACGGAAGGTTTAAGAGCTCGCTGATCAGCCTCGATAAGA
TACATTGATGAGTTTGGACAAACCACAACTAGAATGCAGTGAAAAAA
ATGCTTTATTTGTGAAATTTGTGATGCTATTGCTTTATTTGTAACCA
TTATAAGCTGCAATAAACAAGTTAAGCTTGAATTCAGCTGACGTGCC
TCGGACCGCTAGGAACCCCTAGTGATGGAGTTGGCCACTCCCTCTCT
GCGCGCTCGCTCGCTCACTGAGGCCGGGCGACCAAAGGTCGCCCGAC
GCCCGGGCTTTGCCCGGGCGGCCTCAGTGAGCGAGCGAGCGCGCAGC
TGCCTGCAGGGGCGCCTGATGCGGTATTTTCTCCTTACGCATCTGTG
CGGTATTTCACACCGCATACGTCAAAGCAACCATAGTACGCGCCCTG
TAGCGGCGCATTAAGCGCGGCGGGTGTGGTGGTTACGCGCAGCGTGA
CCGCTACACTTGCCAGCGCCCTAGCGCCCGCTCCTTTCGCTTTCTTC
CCTTCCTTTCTCGCCACGTTCGCCGGCTTTCCCCGTCAAGCTCTAAA
TCGGGGGCTCCCTTTAGGGTTCCGATTTAGTGCTTTACGGCACCTCG
ACCCCAAAAAACTTGATTTGGGTGATGGTTCACGTAGTGGGCCATCG
CCCTGATAGACGGTTTTTCGCCCTTTGACGTTGGAGTCCACGTTCTT
TAATAGTGGACTCTTGTTCCAAACTGGAACAACACTCAACCCTATCT
CGGGCTATTCTTTTGATTTATAAGGGATTTTGCCGATTTCGGCCTAT
TGGTTAAAAAATGAGCTGATTTAACAAAAATTTAACGCGAATTTTAA
CAAAATATTAACGTTTACAATTTTATGGTGCACTCTCAGTACAATCT
GCTCTGATGCCGCATAGTTAAGCCAGCCCCGACACCCGCCAACACCC
GCTGACGCGCCCTGACGGGCTTGTCTGCTCCCGGCATCCGCTTACAG
ACAAGCTGTGACCGTCTCCGGGAGCTGCATGTGTCAGAGGTTTTCAC
CGTCATCACCGAAACGCGCGAGACGAAAGGGCCTCGTGATACGCCTA
TTTTTATAGGTTAATGTCATGAACAATAAAACTGTCTGCTTACATAA
ACAGTAATACAAGGGGTGTTATGAGCCATATTCAACGGGAAACGTCG
AGGCCGCGATTAAATTCCAACATGGATGCTGATTTATATGGGTATAA
ATGGGCTCGCGATAATGTCGGGCAATCAGGTGCGACAATCTATCGCT
TGTATGGGAAGCCCGATGCGCCAGAGTTGTTTCTGAAACATGGCAAA
GGTAGCGTTGCCAATGATGTTACAGATGAGATGGTCAGACTAAACTG
GCTGACGGAATTTATGCCTCTTCCGACCATCAAGCATTTTATCCGTA
CTCCTGATGATGCATGGTTACTCACCACTGCGATCCCCGGAAAAACA
GCATTCCAGGTATTAGAAGAATATCCTGATTCAGGTGAAAATATTGT
TGATGCGCTGGCAGTGTTCCTGCGCCGGTTGCATTCGATTCCTGTTT
GTAATTGTCCTTTTAACAGCGATCGCGTATTTCGTCTCGCTCAGGCG
CAATCACGAATGAATAACGGTTTGGTTGATGCGAGTGATTTTGATGA
CGAGCGTAATGGCTGGCCTGTTGAACAAGTCTGGAAAGAAATGCATA
AACTTTTGCCATTCTCACCGGATTCAGTCGTCACTCATGGTGATTTC
TCACTTGATAACCTTATTTTTGACGAGGGGAAATTAATAGGTTGTAT
TGATGTTGGACGAGTCGGAATCGCAGACCGATACCAGGATCTTGCCA
TCCTATGGAACTGCCTCGGTGAGTTTTCTCCTTCATTACAGAAACGG
CTTTTTCAAAAATATGGTATTGATAATCCTGATATGAATAAATTGCA
GTTTCATTTGATGCTCGATGAGTTTTTCTAATCTCATGACCAAAATC
CCTTAACGTGAGTTTTCGTTCCACTGAGCGTCAGACCCCGTAGAAAA
GATCAAAGGATCTTCTTGAGATCCTTTTTTTCTGCGCGTAATCTGCT
GCTTGCAAACAAAAAAACCACCGCTACCAGCGGTGGTTTGTTTGCCG
GATCAAGAGCTACCAACTCTTTTTCCGAAGGTAACTGGCTTCAGCAG
AGCGCAGATACCAAATACTGTCCTTCTAGTGTAGCCGTAGTTAGGCC
ACCACTTCAAGAACTCTGTAGCACCGCCTACATACCTCGCTCTGCTA
ATCCTGTTACCAGTGGCTGCTGCCAGTGGCGATAAGTCGTGTCTTAC
CGGGTTGGACTCAAGACGATAGTTACCGGATAAGGCGCAGCGGTCGG
GCTGAACGGGGGGTTCGTGCACACAGCCCAGCTTGGAGCGAACGAGC
TACACCGAACTGAGATACCTACAGCGTGAGCTATGAGAAAGCGCCAC
GCTTCCCGAAGGGAGAAAGGCGGACAGGTATCCGGTAAGCGGCAGGG
TCGGAACAGGAGAGCGCACGAGGGAGCTTCCAGGGGGAAACGCCTGG
TATCTTTATAGTCCTGTCGGGTTTCGCCACCTCTGACTTGAGCGTCG
ATTTTTGTGATGCTCGTCAGGGGGGCGGAGCCTATGGAAAAACGCCA
GCAACGCGGCCTTTTTACGGTTCCTGGCCTTTTGCTGGCCTTTTGCT
CACATGT

Exemplary Cas9 with GFP and sgRNA Plasmids

Exemplary pITR-CMV.SaCas9T2AGFP.U6-hsa408Rev
sequence
(SEQ ID NO: 281)
CTGCGCGCTCGCTCGCTCACTGAGGCCGCCCGGGCGTCGGGCGACCT
TTGGTCGCCCGGCCTCAGTGAGCGAGCGAGCGCGCAGAGAGGGAGTG
GCCAACTCCATCACTAGGGGTTCCTGCGGCCGCACGCGTCTAGATCC
CATATATGGAGTTCCGCGTTACATAACTTACGGTAAATGGCCCGCCT
GGCTGACCGCCCAACGACCCCCGCCCATTGACGTCAATAATGACGTA
TGTTCCCATAGTAACGCCAATAGGGACTTTCCATTGACGTCAATGGG
TGGAGTATTTACGGTAAACTGCCCACTTGGCAGTACATCAAGTGTAT
CATATGCCAAGTACGCCCCCTATTGACGTCAATGACGGTAAATGGCC
CGCCTGGCATTATGCCCAGTACATGACCTTATGGGACTTTCCTACTT
GGCAGTACATCTACGTATTAGTCATCGCTATTACCATGGTGATGCGG
TTTTGGCAGTACATCAATGGGCGTGGATAGCGGTTTGACTCACGGGG
ATTTCCAAGTCTCCACCCCATTGACGTCAATGGGAGTTTGTTTTGGC
ACCAAAATCAACGGGACTTTCCAAAATGTCGTAACAACTCCGCCCCA
TTGACGCAAATGGGCGGTAGGCGTGTACGGTGGGAGGTCTATATAAG
CAGAGCTCGTTTAGTGAACCGTcagatcgcctggagacgcACCGGTG
CCACCATGGCCCCTAAGAAGAAGCGGAAAGTCGGCAGCGGCAAGCGG
AACTATATCCTGGGCCTCGACATCGGCATCACCAGCGTTGGCTACGG
CATCATCGACTACGAGACACGGGACGTGATCGATGCCGGCGTGCGGC
TGTTTAAAGAGGCCAACGTCGAGAACAACGAGGGCCGCAGATCTAAG
AGAGGCGCCAGACGGCTGAAGCGGAGAAGAAGGCACAGAATCCAGAG
AGTGAAGAAGCTGCTGTTCGACTACAACCTGCTGACCGACCACAGCG
AGCTGAGCGGCATCAATCCTTACGAGGCCAGAGTGAAGGGCCTGAGC
CAGAAGCTGAGCGAGGAAGAGTTTAGCGCCGCTCTGCTGCACCTGGC
CAAGAGAAGAGGCGTGCACAACGTGAACGAGGTGGAAGAGGACACCG
GCAACGAGCTGTCCACCAAAGAGCAGATCAGCCGGAACAGCAAGGCC
CTGGAAGAGAAATACGTGGCCGAACTGCAGCTGGAACGGCTGAAAAA
GGATGGCGAAGTGCGGGGCAGCATCAATCGGTTCAAGACCAGCGACT
ACGTGAAAGAAGCCAAACAGCTGCTGAAGGTGCAGAAGGCCTACCAC
CAGCTGGACCAGAGCTTCATCGACACCTACATCGACCTGCTGGAAAC
CCGGCGGACCTACTATGAAGGACCTGGCGAGGGAAGCCCCTTCGGCT
GGAAGGACATCAAAGAATGGTACGAGATGCTGATGGGCCACTGCACC
TACTTTCCCGAGGAACTGCGGAGCGTGAAGTACGCCTACAACGCCGA
CCTGTACAACGCCCTGAACGACCTGAACAACCTCGTGATCACCCGGG
AGGAGAACGAGAAGCTGGAATATTAGGAGAAGTTCCAGATCATCGAG
AACGTGTTCAAGCAGAAGAAGAAACCCACGCTGAAGCAGATCGCCAA
AGAGATCCTGGTCAACGAGGAAGATATCAAGGGCTACAGAGTGACCA
GCACCGGCAAGCCCGAGTTCACCAACCTGAAGGTGTACCACGACATC
AAGGATATCACAGCCCGGAAAGAGATTATTGAGAACGCCGAGCTGCT
GGACCAAATCGCCAAGATCCTGACCATCTACCAGAGCAGCGAGGACA
TCCAAGAGGAACTGACCAATCTGAACTCCGAGCTGACCCAAGAAGAG
ATCGAGCAGATCTCTAATCTGAAGGGGTACACCGGCACACACAACCT
GAGCCTGAAGGCCATCAACCTGATCCTGGACGAGCTGTGGCACACCA
ACGACAACCAGATTGCCATCTTCAACAGGCTGAAGCTGGTGCCCAAG
AAGGTGGACCTGTCTCAGCAGAAAGAAATCCCCACCACACTGGTGGA
CGACTTCATTCTGAGCCCCGTGGTCAAGAGGAGCTTCATCCAGAGCA
TCAAAGTGATCAACGCCATCATCAAGAAGTACGGGCTGCCCAACGAT
ATCATCATCGAGCTGGCCCGCGAGAAGAACTCCAAGGACGCCCAGAA
AATGATCAACGAGATGCAGAAGAGGAACCGCCAGACCAACGAGCGGA
TCGAGGAAATCATCCGGACCACCGGCAAAGAGAACGCCAAGTACCTG
ATCGAGAAGATCAAGCTGCACGACATGCAAGAGGGCAAGTGCCTGTA
CAGCCTGGAAGCCATTCCTCTGGAAGATCTGCTGAACAATCCCTTCA
ACTATGAGGTGGACCACATCATCCCCAGAAGCGTGTCCTTCGACAAC
AGCTTCAACAACAAGGTGCTCGTGAAGCAAGAGGAAAACAGCAAGAA
GGGCAACAGAACCCCATTCCAGTACCTGAGCAGCAGCGACAGCAAGA
TCAGCTACGAAACCTTCAAGAAGCACATCCTGAATCTGGCCAAAGGC
AAGGGCAGAATCAGCAAGACCAAGAAAGAATACCTGCTCGAGGAACG
GGACATCAACAGATTCAGCGTGCAGAAAGACTTCATCAATCGGAACC
TGGTGGACACCAGATACGCCACCAGAGGCCTGATGAATCTGCTGCGG
AGCTACTTCAGAGTGAACAATCTGGACGTGAAAGTCAAGTCCATCAA
CGGCGGCTTCACCAGCTTTCTGCGGCGGAAGTGGAAGTTCAAGAAAG
AGCGGAACAAGGGCTATAAGCACCACGCCGAGGACGCCCTGATCATT
GCCAACGCCGATTTCATCTTCAAAGAGTGGAAGAAACTGGACAAGGC
CAAAAAAGTGATGGAAAACCAGATGTTCGAGGAAAAGCAGGCCGAGA
GCATGCCCGAGATCGAAACCGAGCAAGAGTACAAAGAGATTTTCATC
ACGCCCCACCAGATCAAGCACATTAAGGACTTCAAGGACTACAAGTA
CAGCCACCGCGTGGACAAGAAGCCCAATAGAGAGCTGATTAACGACA
CCCTGTACTCTACCCGGAAGGACGACAAGGGCAATACCCTGATCGTC
AACAACCTGAACGGCCTGTACGACAAGGACAACGACAAGCTCAAGAA
GCTGATCAACAAGAGCCCCGAGAAACTGCTGATGTACCACCACGATC
CTCAGACCTACCAGAAACTGAAGCTCATCATGGAACAGTACGGCGAC
GAGAAGAATCCCCTGTACAAGTACTACGAGGAAACCGGGAACTACCT
GACCAAGTACTCCAAAAAGGACAATGGGCCCGTGATCAAGAAGATTA
AGTATTAGGGCAACAAGCTGAATGCCCACCTGGACATCACCGACGAC
TACCCCAACAGCAGAAACAAGGTGGTCAAGCTGTCCCTGAAGCCTTA
CAGATTCGACGTGTACCTGGACAACGGCGTGTACAAGTTCGTGACCG
TGAAGAACCTGGACGTCATCAAAAAAGAAAACTACTAGGAAGTCAAC
AGCAAGTGCTATGAGGAAGCCAAGAAACTCAAGAAAATCAGCAACCA
GGCCGAGTTTATCGCCTCCTTCTACAACAACGATCTGATCAAGATCA
ACGGGGAGCTGTATAGAGTGATCGGCGTGAACAATGACCTGCTGAAC
CGGATCGAAGTGAACATGATCGACATCACCTACCGCGAGTACCTCGA
GAACATGAACGATAAGCGGCCTCCACGGATCATTAAGACAATCGCCA
GCAAGACGCAGAGCATTAAGAAGTACAGCACAGACATCCTGGGCAAC
CTGTACGAAGTGAAGTCTAAGAAGCACCCGCAGATTATCAAGAAAGG
CAGCGGCGCTCCCAAGAAAAAACGGAAGGTTGGCTCCGGAGAGGGCA
GAGGAAGTCTGCTAACATGCGGTGACGTCGAGGAGAATCCTGGCCCA
ATGGAGAGCGACGAGAGCGGCCTGCCCGCCATGGAGATCGAGTGCCG
CATCACCGGCACCCTGAACGGCGTGGAGTTCGAGCTGGTGGGCGGCG
GAGAGGGCACCCCCGAGCAGGGCCGCATGACCAACAAGATGAAGAGC
ACCAAAGGCGCCCTGACCTTCAGCCCCTACCTGCTGAGCCACGTGAT
GGGCTACGGCTTCTACCACTTCGGCACCTACCCCAGCGGCTACGAGA
ACCCCTTCCTGCACGCCATCAACAACGGCGGCTACACCAACACCCGC
ATCGAGAAGTACGAGGACGGCGGCGTGCTGCACGTGAGCTTCAGCTA
CCGCTACGAGGCCGGCCGCGTGATCGGCGACTTCAAGGTGATGGGCA
CCGGCTTCCCCGAGGACAGCGTGATCTTCACCGACAAGATCATCCGC
AGCAACGCCACCGTGGAGCACCTGCACCCCATGGGCGATAACGATCT
GGATGGCAGCTTCACCCGCACCTTCAGCCTGCGCGACGGCGGCTACT
ACAGCTCCGTGGTGGACAGCCACATGCACTTCAAGAGCGCCATCCAC
CCCAGCATCCTGCAGAACGGGGGCCCCATGTTCGCCTTCCGCCGCGT
GGAGGAGGATCACAGCAACACCGAGCTGGGCATCGTGGAGTACCAGC
ACGCCTTCAAGACCCCGGATGCAGATGCCGGTGAAGAATAAGAGCTC
GCTGATCAGCCTCGATAAGATACATTGATGAGTTTGGACAAACCACA
ACTAGAATGCAGTGAAAAAAATGCTTTATTTGTGAAATTTGTGATGC
TATTGCTTTATTTGTAACCATTATAAGCTGCAATAAACAAGTTTTAA
TTAAGGTCGGGCAGGAAGAGGGCCTATTTCCCATGATTCCTTCATAT
TTGCATATACGATACAAGGCTGTTAGAGAGATAATTAGAATTAATTT
GACTGTAAACACAAAGATATTAGTACAAAATACGTGACGTAGAAAGT
AATAATTTCTTGGGTAGTTTGCAGTTTTAAAATTATGTTTTAAAATG
GACTATCATATGCTTACCGTAACTTGAAAGTATTTCGATTTCTTGGC
TTTATATATCTTGTGGAAAGGACGAAACACCGAAGCCGAAAACCACG
ATCATCGTTTAAGTACTCTGTGCTGGAAACAGCACAGAATCTACTTA
AACAAGGCAAAATGCCGTGTTTATCTCGTCAACTTGTTGGCGAGATT
TTttctaAAGCTTGAATTCAGCTGACGTGCCTCGGACCGCTAGGAAC
CCCTAGTGATGGAGTTGGCCACTCCCTCTCTGCGCGCTCGCTCGCTC
ACTGAGGCCGGGCGACCAAAGGTCGCCCGACGCCCGGGCTTTGCCCG
GGCGGCCTCAGTGAGCGAGCGAGCGCGCAG
Exemplary pITR-CMV.deadSaCas9T2AGFP.U6-hsa408Rev
sequence
(SEQ ID NO: 282)
CTGCGCGCTCGCTCGCTCACTGAGGCCGCCCGGGCGTCGGGCGACCT
TTGGTCGCCCGGCCTCAGTGAGCGAGCGAGCGCGCAGAGAGGGAGTG
GCCAACTCCATCACTAGGGGTTCCTGCGGCCGCACGCGTCTAGATCC
CATATATGGAGTTCCGCGTTACATAACTTACGGTAAATGGCCCGCCT
GGCTGACCGCCCAACGACCCCCGCCCATTGACGTCAATAATGACGTA
TGTTCCCATAGTAACGCCAATAGGGACTTTCCATTGACGTCAATGGG
TGGAGTATTTACGGTAAACTGCCCACTTGGCAGTACATCAAGTGTAT
CATATGCCAAGTACGCCCCCTATTGACGTCAATGACGGTAAATGGCC
CGCCTGGCATTATGCCCAGTACATGACCTTATGGGACTTTCCTACTT
GGCAGTACATCTACGTATTAGTCATCGCTATTACCATGGTGATGCGG
TTTTGGCAGTACATCAATGGGCGTGGATAGCGGTTTGACTCACGGGG
ATTTCCAAGTCTCCACCCCATTGACGTCAATGGGAGTTTGTTTTGGC
ACCAAAATCAACGGGACTTTCCAAAATGTCGTAACAACTCCGCCCCA
TTGACGCAAATGGGCGGTAGGCGTGTACGGTGGGAGGTCTATATAAG
CAGAGCTCGTTTAGTGAACCGTcagatcgcctggagacgcACCGGTG
CCACCATGGCCCCTAAGAAGAAGCGGAAAGTCGGCAGCGGCAAGCGG
AACTATATCCTGGGCCTCGCCATCGGCATCACCAGCGTTGGCTACGG
CATCATCGACTACGAGACACGGGACGTGATCGATGCCGGCGTGCGGC
TGTTTAAAGAGGCCAACGTCGAGAACAACGAGGGCCGCAGATCTAAG
AGAGGCGCCAGACGGCTGAAGCGGAGAAGAAGGCACAGAATCCAGAG
AGTGAAGAAGCTGCTGTTCGACTACAACCTGCTGACCGACCACAGCG
AGCTGAGCGGCATCAATCCTTACGAGGCCAGAGTGAAGGGCCTGAGC
CAGAAGCTGAGCGAGGAAGAGTTTAGCGCCGCTCTGCTGCACCTGGC
CAAGAGAAGAGGCGTGCACAACGTGAACGAGGTGGAAGAGGACACCG
GCAACGAGCTGTCCACCAAAGAGCAGATCAGCCGGAACAGCAAGGCC
CTGGAAGAGAAATACGTGGCCGAACTGCAGCTGGAACGGCTGAAAAA
GGATGGCGAAGTGCGGGGCAGCATCAATCGGTTCAAGACCAGCGACT
ACGTGAAAGAAGCCAAACAGCTGCTGAAGGTGCAGAAGGCCTACCAC
CAGCTGGACCAGAGCTTCATCGACACCTACATCGACCTGCTGGAAAC
CCGGCGGACCTACTATGAAGGACCTGGCGAGGGAAGCCCCTTCGGCT
GGAAGGACATCAAAGAATGGTACGAGATGCTGATGGGCCACTGCACC
TACTTTCCCGAGGAACTGCGGAGCGTGAAGTACGCCTACAACGCCGA
CCTGTACAACGCCCTGAACGACCTGAACAACCTCGTGATCACCCGGG
ACGAGAACGAGAAGCTGGAATATTAGGAGAAGTTCCAGATCATCGAG
AACGTGTTCAAGCAGAAGAAGAAACCCACGCTGAAGCAGATCGCCAA
AGAGATCCTGGTCAACGAGGAAGATATCAAGGGCTACAGAGTGACCA
GCACCGGCAAGCCCGAGTTCACCAACCTGAAGGTGTACCACGACATC
AAGGATATCACAGCCCGGAAAGAGATTATTGAGAACGCCGAGCTGCT
GGACCAAATCGCCAAGATCCTGACCATCTACCAGAGCAGCGAGGACA
TCCAAGAGGAACTGACCAATCTGAACTCCGAGCTGACCCAAGAAGAG
ATCGAGCAGATCTCTAATCTGAAGGGGTACACCGGCACACACAACCT
GAGCCTGAAGGCCATCAACCTGATCCTGGACGAGCTGTGGCACACCA
ACGACAACCAGATTGCCATCTTCAACAGGCTGAAGCTGGTGCCCAAG
AAGGTGGACCTGTCTCAGCAGAAAGAAATCCCCACCACACTGGTGGA
CGACTTCATTCTGAGCCCCGTGGTCAAGAGGAGCTTCATCCAGAGCA
TCAAAGTGATCAACGCCATCATCAAGAAGTACGGGCTGCCCAACGAT
ATCATCATCGAGCTGGCCCGCGAGAAGAACTCCAAGGACGCCCAGAA
AATGATCAACGAGATGCAGAAGAGGAACCGCCAGACCAACGAGCGGA
TCGAGGAAATCATCCGGACCACCGGCAAAGAGAACGCCAAGTACCTG
ATCGAGAAGATCAAGCTGCACGACATGCAAGAGGGCAAGTGCCTGTA
CAGCCTGGAAGCCATTCCTCTGGAAGATCTGCTGAACAATCCCTTCA
ACTATGAGGTGGACCACATCATCCCCAGAAGCGTGTCCTTCGACAAC
AGCTTCAACAACAAGGTGCTCGTGAAGCAAGAGGAAGCCAGCAAGAA
GGGCAACAGAACCCCATTCCAGTACCTGAGCAGCAGCGACAGCAAGA
TCAGCTACGAAACCTTCAAGAAGCACATCCTGAATCTGGCCAAAGGC
AAGGGCAGAATCAGCAAGACCAAGAAAGAATACCTGCTCGAGGAACG
GGACATCAACAGATTCAGCGTGCAGAAAGACTTCATCAATCGGAACC
TGGTGGACACCAGATACGCCACCAGAGGCCTGATGAATCTGCTGCGG
AGCTACTTCAGAGTGAACAATCTGGACGTGAAAGTCAAGTCCATCAA
CGGCGGCTTCACCAGCTTTCTGCGGCGGAAGTGGAAGTTCAAGAAAG
AGCGGAACAAGGGCTATAAGCACCACGCCGAGGACGCCCTGATCATT
GCCAACGCCGATTTCATCTTCAAAGAGTGGAAGAAACTGGACAAGGC
CAAAAAAGTGATGGAAAACCAGATGTTCGAGGAAAAGCAGGCCGAGA
GCATGCCCGAGATCGAAACCGAGCAAGAGTACAAAGAGATTTTCATC
ACGCCCCACCAGATCAAGCACATTAAGGACTTCAAGGACTACAAGTA
CAGCCACCGCGTGGACAAGAAGCCCAATAGAGAGCTGATTAACGACA
CCCTGTACTCTACCCGGAAGGACGACAAGGGCAATACCCTGATCGTC
AACAACCTGAACGGCCTGTACGACAAGGACAACGACAAGCTCAAGAA
GCTGATCAACAAGAGCCCCGAGAAACTGCTGATGTACCACCACGATC
CTCAGACCTACCAGAAACTGAAGCTCATCATGGAACAGTACGGCGAC
GAGAAGAATCCCCTGTACAAGTACTACGAGGAAACCGGGAACTACCT
GACCAAGTACTCCAAAAAGGACAATGGGCCCGTGATCAAGAAGATTA
AGTATTAGGGCAACAAGCTGAATGCCCACCTGGACATCACCGACGAC
TACCCCAACAGCAGAAACAAGGTGGTCAAGCTGTCCCTGAAGCCTTA
CAGATTCGACGTGTACCTGGACAACGGCGTGTACAAGTTCGTGACCG
TGAAGAACCTGGACGTCATCAAAAAAGAAAACTACTAGGAAGTCAAC
AGCAAGTGCTATGAGGAAGCCAAGAAACTCAAGAAAATCAGCAACCA
GGCCGAGTTTATCGCCTCCTTCTACAACAACGATCTGATCAAGATCA
ACGGGGAGCTGTATAGAGTGATCGGCGTGAACAATGACCTGCTGAAC
CGGATCGAAGTGAACATGATCGACATCACCTACCGCGAGTACCTCGA
GAACATGAACGATAAGCGGCCTCCACGGATCATTAAGACAATCGCCA
GCAAGACGCAGAGCATTAAGAAGTACAGCACAGACATCCTGGGCAAC
CTGTACGAAGTGAAGTCTAAGAAGCACCCGCAGATTATCAAGAAAGG
CAGCGGCGCTCCCAAGAAAAAACGGAAGGTTGGCTCCGGAGAGGGCA
GAGGAAGTCTGCTAACATGCGGTGACGTCGAGGAGAATCCTGGCCCA
ATGGAGAGCGACGAGAGCGGCCTGCCCGCCATGGAGATCGAGTGCCG
CATCACCGGCACCCTGAACGGCGTGGAGTTCGAGCTGGTGGGCGGCG
GAGAGGGCACCCCCGAGCAGGGCCGCATGACCAACAAGATGAAGAGC
ACCAAAGGCGCCCTGACCTTCAGCCCCTACCTGCTGAGCCACGTGAT
GGGCTACGGCTTCTACCACTTCGGCACCTACCCCAGCGGCTACGAGA
ACCCCTTCCTGCACGCCATCAACAACGGCGGCTACACCAACACCCGC
ATCGAGAAGTACGAGGACGGCGGCGTGCTGCACGTGAGCTTCAGCTA
CCGCTACGAGGCCGGCCGCGTGATCGGCGACTTCAAGGTGATGGGCA
CCGGCTTCCCCGAGGACAGCGTGATCTTCACCGACAAGATCATCCGC
AGCAACGCCACCGTGGAGCACCTGCACCCCATGGGCGATAACGATCT
GGATGGCAGCTTCACCCGCACCTTCAGCCTGCGCGACGGCGGCTACT
ACAGCTCCGTGGTGGACAGCCACATGCACTTCAAGAGCGCCATCCAC
CCCAGCATCCTGCAGAACGGGGGCCCCATGTTCGCCTTCCGCCGCGT
GGAGGAGGATCACAGCAACACCGAGCTGGGCATCGTGGAGTACCAGC
ACGCCTTCAAGACCCCGGATGCAGATGCCGGTGAAGAATAAGAGCTC
GCTGATCAGCCTCGATAAGATACATTGATGAGTTTGGACAAACCACA
ACTAGAATGCAGTGAAAAAAATGCTTTATTTGTGAAATTTGTGATGC
TATTGCTTTATTTGTAACCATTATAAGCTGCAATAAACAAGTTTTAA
TTAAGGTCGGGCAGGAAGAGGGCCTATTTCCCATGATTCCTTCATAT
TTGCATATACGATACAAGGCTGTTAGAGAGATAATTAGAATTAATTT
GACTGTAAACACAAAGATATTAGTACAAAATACGTGACGTAGAAAGT
AATAATTTCTTGGGTAGTTTGCAGTTTTAAAATTATGTTTTAAAATG
GACTATCATATGCTTACCGTAACTTGAAAGTATTTCGATTTCTTGGC
TTTATATATCTTGTGGAAAGGACGAAACACCGAAGCCGAAAACCACG
ATCATCGTTTAAGTACTCTGTGCTGGAAACAGCACAGAATCTACTTA
AACAAGGCAAAATGCCGTGTTTATCTCGTCAACTTGTTGGCGAGATT
TTTTCTAAAGCTTGAATTCAGCTGACGTGCCTCGGACCGCTAGGAAC
CCCTAGTGATGGAGTTGGCCACTCCCTCTCTGCGCGCTCGCTCGCTC
ACTGAGGCCGGGCGACCAAAGGTCGCCCGACGCCCGGGCTTTGCCCG
GGCGGCCTCAGTGAGCGAGCGAGCGCGCAG
Exemplary pITR-CMV.SaCas9T2AGFP.U6-hsammu386Fw
sequence
(SEQ ID NO: 283)
CTGCGCGCTCGCTCGCTCACTGAGGCCGCCCGGGCGTCGGGCGACCT
TTGGTCGCCCGGCCTCAGTGAGCGAGCGAGCGCGCAGAGAGGGAGTG
GCCAACTCCATCACTAGGGGTTCCTGCGGCCGCACGCGTCTAGATCC
CATATATGGAGTTCCGCGTTACATAACTTACGGTAAATGGCCCGCCT
GGCTGACCGCCCAACGACCCCCGCCCATTGACGTCAATAATGACGTA
TGTTCCCATAGTAACGCCAATAGGGACTTTCCATTGACGTCAATGGG
TGGAGTATTTACGGTAAACTGCCCACTTGGCAGTACATCAAGTGTAT
CATATGCCAAGTACGCCCCCTATTGACGTCAATGACGGTAAATGGCC
CGCCTGGCATTATGCCCAGTACATGACCTTATGGGACTTTCCTACTT
GGCAGTACATCTACGTATTAGTCATCGCTATTACCATGGTGATGCGG
TTTTGGCAGTACATCAATGGGCGTGGATAGCGGTTTGACTCACGGGG
ATTTCCAAGTCTCCACCCCATTGACGTCAATGGGAGTTTGTTTTGGC
ACCAAAATCAACGGGACTTTCCAAAATGTCGTAACAACTCCGCCCCA
TTGACGCAAATGGGCGGTAGGCGTGTACGGTGGGAGGTCTATATAAG
CAGAGCTCGTTTAGTGAACCGTcagatcgcctggagacgcACCGGTG
CCACCATGGCCCCTAAGAAGAAGCGGAAAGTCGGCAGCGGCAAGCGG
AACTATATCCTGGGCCTCGACATCGGCATCACCAGCGTTGGCTACGG
CATCATCGACTACGAGACACGGGACGTGATCGATGCCGGCGTGCGGC
TGTTTAAAGAGGCCAACGTCGAGAACAACGAGGGCCGCAGATCTAAG
AGAGGCGCCAGACGGCTGAAGCGGAGAAGAAGGCACAGAATCCAGAG
AGTGAAGAAGCTGCTGTTCGACTACAACCTGCTGACCGACCACAGCG
AGCTGAGCGGCATCAATCCTTACGAGGCCAGAGTGAAGGGCCTGAGC
CAGAAGCTGAGCGAGGAAGAGTTTAGCGCCGCTCTGCTGCACCTGGC
CAAGAGAAGAGGCGTGCACAACGTGAACGAGGTGGAAGAGGACACCG
GCAACGAGCTGTCCACCAAAGAGCAGATCAGCCGGAACAGCAAGGCC
CTGGAAGAGAAATACGTGGCCGAACTGCAGCTGGAACGGCTGAAAAA
GGATGGCGAAGTGCGGGGCAGCATCAATCGGTTCAAGACCAGCGACT
ACGTGAAAGAAGCCAAACAGCTGCTGAAGGTGCAGAAGGCCTACCAC
CAGCTGGACCAGAGCTTCATCGACACCTACATCGACCTGCTGGAAAC
CCGGCGGACCTACTATGAAGGACCTGGCGAGGGAAGCCCCTTCGGCT
GGAAGGACATCAAAGAATGGTACGAGATGCTGATGGGCCACTGCACC
TACTTTCCCGAGGAACTGCGGAGCGTGAAGTACGCCTACAACGCCGA
CCTGTACAACGCCCTGAACGACCTGAACAACCTCGTGATCACCCGGG
ACGAGAACGAGAAGCTGGAATATTAGGAGAAGTTCCAGATCATCGAG
AACGTGTTCAAGCAGAAGAAGAAACCCACGCTGAAGCAGATCGCCAA
AGAGATCCTGGTCAACGAGGAAGATATCAAGGGCTACAGAGTGACCA
GCACCGGCAAGCCCGAGTTCACCAACCTGAAGGTGTACCACGACATC
AAGGATATCACAGCCCGGAAAGAGATTATTGAGAACGCCGAGCTGCT
GGACCAAATCGCCAAGATCCTGACCATCTACCAGAGCAGCGAGGACA
TCCAAGAGGAACTGACCAATCTGAACTCCGAGCTGACCCAAGAAGAG
ATCGAGCAGATCTCTAATCTGAAGGGGTACACCGGCACACACAACCT
GAGCCTGAAGGCCATCAACCTGATCCTGGACGAGCTGTGGCACACCA
ACGACAACCAGATTGCCATCTTCAACAGGCTGAAGCTGGTGCCCAAG
AAGGTGGACCTGTCTCAGCAGAAAGAAATCCCCACCACACTGGTGGA
CGACTTCATTCTGAGCCCCGTGGTCAAGAGGAGCTTCATCCAGAGCA
TCAAAGTGATCAACGCCATCATCAAGAAGTACGGGCTGCCCAACGAT
ATCATCATCGAGCTGGCCCGCGAGAAGAACTCCAAGGACGCCCAGAA
AATGATCAACGAGATGCAGAAGAGGAACCGCCAGACCAACGAGCGGA
TCGAGGAAATCATCCGGACCACCGGCAAAGAGAACGCCAAGTACCTG
ATCGAGAAGATCAAGCTGCACGACATGCAAGAGGGCAAGTGCCTGTA
CAGCCTGGAAGCCATTCCTCTGGAAGATCTGCTGAACAATCCCTTCA
ACTATGAGGTGGACCACATCATCCCCAGAAGCGTGTCCTTCGACAAC
AGCTTCAACAACAAGGTGCTCGTGAAGCAAGAGGAAAACAGCAAGAA
GGGCAACAGAACCCCATTCCAGTACCTGAGCAGCAGCGACAGCAAGA
TCAGCTACGAAACCTTCAAGAAGCACATCCTGAATCTGGCCAAAGGC
AAGGGCAGAATCAGCAAGACCAAGAAAGAATACCTGCTCGAGGAACG
GGACATCAACAGATTCAGCGTGCAGAAAGACTTCATCAATCGGAACC
TGGTGGACACCAGATACGCCACCAGAGGCCTGATGAATCTGCTGCGG
AGCTACTTCAGAGTGAACAATCTGGACGTGAAAGTCAAGTCCATCAA
CGGCGGCTTCACCAGCTTTCTGCGGCGGAAGTGGAAGTTCAAGAAAG
AGCGGAACAAGGGCTATAAGCACCACGCCGAGGACGCCCTGATCATT
GCCAACGCCGATTTCATCTTCAAAGAGTGGAAGAAACTGGACAAGGC
CAAAAAAGTGATGGAAAACCAGATGTTCGAGGAAAAGCAGGCCGAGA
GCATGCCCGAGATCGAAACCGAGCAAGAGTACAAAGAGATTTTCATC
ACGCCCCACCAGATCAAGCACATTAAGGACTTCAAGGACTACAAGTA
CAGCCACCGCGTGGACAAGAAGCCCAATAGAGAGCTGATTAACGACA
CCCTGTACTCTACCCGGAAGGACGACAAGGGCAATACCCTGATCGTC
AACAACCTGAACGGCCTGTACGACAAGGACAACGACAAGCTCAAGAA
GCTGATCAACAAGAGCCCCGAGAAACTGCTGATGTACCACCACGATC
CTCAGACCTACCAGAAACTGAAGCTCATCATGGAACAGTACGGCGAC
GAGAAGAATCCCCTGTACAAGTACTACGAGGAAACCGGGAACTACCT
GACCAAGTACTCCAAAAAGGACAATGGGCCCGTGATCAAGAAGATTA
AGTATTAGGGCAACAAGCTGAATGCCCACCTGGACATCACCGACGAC
TACCCCAACAGCAGAAACAAGGTGGTCAAGCTGTCCCTGAAGCCTTA
CAGATTCGACGTGTACCTGGACAACGGCGTGTACAAGTTCGTGACCG
TGAAGAACCTGGACGTCATCAAAAAAGAAAACTACTAGGAAGTCAAC
AGCAAGTGCTATGAGGAAGCCAAGAAACTCAAGAAAATCAGCAACCA
GGCCGAGTTTATCGCCTCCTTCTACAACAACGATCTGATCAAGATCA
ACGGGGAGCTGTATAGAGTGATCGGCGTGAACAATGACCTGCTGAAC
CGGATCGAAGTGAACATGATCGACATCACCTACCGCGAGTACCTCGA
GAACATGAACGATAAGCGGCCTCCACGGATCATTAAGACAATCGCCA
GCAAGACGCAGAGCATTAAGAAGTACAGCACAGACATCCTGGGCAAC
CTGTACGAAGTGAAGTCTAAGAAGCACCCGCAGATTATCAAGAAAGG
CAGCGGCGCTCCCAAGAAAAAACGGAAGGTTGGCTCCGGAGAGGGCA
GAGGAAGTCTGCTAACATGCGGTGACGTCGAGGAGAATCCTGGCCCA
ATGGAGAGCGACGAGAGCGGCCTGCCCGCCATGGAGATCGAGTGCCG
CATCACCGGCACCCTGAACGGCGTGGAGTTCGAGCTGGTGGGCGGCG
GAGAGGGCACCCCCGAGCAGGGCCGCATGACCAACAAGATGAAGAGC
ACCAAAGGCGCCCTGACCTTCAGCCCCTACCTGCTGAGCCACGTGAT
GGGCTACGGCTTCTACCACTTCGGCACCTACCCCAGCGGCTACGAGA
ACCCCTTCCTGCACGCCATCAACAACGGCGGCTACACCAACACCCGC
ATCGAGAAGTACGAGGACGGCGGCGTGCTGCACGTGAGCTTCAGCTA
CCGCTACGAGGCCGGCCGCGTGATCGGCGACTTCAAGGTGATGGGCA
CCGGCTTCCCCGAGGACAGCGTGATCTTCACCGACAAGATCATCCGC
AGCAACGCCACCGTGGAGCACCTGCACCCCATGGGCGATAACGATCT
GGATGGCAGCTTCACCCGCACCTTCAGCCTGCGCGACGGCGGCTACT
ACAGCTCCGTGGTGGACAGCCACATGCACTTCAAGAGCGCCATCCAC
CCCAGCATCCTGCAGAACGGGGGCCCCATGTTCGCCTTCCGCCGCGT
GGAGGAGGATCACAGCAACACCGAGCTGGGCATCGTGGAGTACCAGC
ACGCCTTCAAGACCCCGGATGCAGATGCCGGTGAAGAATAAGAGCTC
GCTGATCAGCCTCGATAAGATACATTGATGAGTTTGGACAAACCACA
ACTAGAATGCAGTGAAAAAAATGCTTTATTTGTGAAATTTGTGATGC
TATTGCTTTATTTGTAACCATTATAAGCTGCAATAAACAAGTTTTAA
TTAAGGTCGGGCAGGAAGAGGGCCTATTTCCCATGATTCCTTCATAT
TTGCATATACGATACAAGGCTGTTAGAGAGATAATTAGAATTAATTT
GACTGTAAACACAAAGATATTAGTACAAAATACGTGACGTAGAAAGT
AATAATTTCTTGGGTAGTTTGCAGTTTTAAAATTATGTTTTAAAATG
GACTATCATATGCTTACCGTAACTTGAAAGTATTTCGATTTCTTGGC
TTTATATATCTTGTGGAAAGGACGAAACACCGAGGAGCACCAGGAAC
TTGCCAGTTTAAGTACTCTGTGCTGGAAACAGCACAGAATCTAGTTA
AACAAGGCAAAATGCCGTGTTTATCTCGTCAACTTGTTGGCGAGATT
TTTTCTAAAGCTTGAATTCAGCTGACGTGCCTCGGACCGCTAGGAAC
CCCTAGTGATGGAGTTGGCCACTCCCTCTCTGCGCGCTCGCTCGCTC
ACTGAGGCCGGGCGACCAAAGGTCGCCCGACGCCCGGGCTTTGCCCG
GGCGGCCTCAGTGAGCGAGCGAGCGCGCAG
Exemplary pITR-CMV.deadSaCas9T2AGFP.U6-hsammu386Fw
sequence
(SEQ ID NO: 284)
CTGCGCGCTCGCTCGCTCACTGAGGCCGCCCGGGCGTCGGGCGACCT
TTGGTCGCCCGGCCTCAGTGAGCGAGCGAGCGCGCAGAGAGGGAGTG
GCCAACTCCATCACTAGGGGTTCCTGCGGCCGCACGCGTCTAGATCC
CATATATGGAGTTCCGCGTTACATAACTTACGGTAAATGGCCCGCCT
GGCTGACCGCCCAACGACCCCCGCCCATTGACGTCAATAATGACGTA
TGTTCCCATAGTAACGCCAATAGGGACTTTCCATTGACGTCAATGGG
TGGAGTATTTACGGTAAACTGCCCACTTGGCAGTACATCAAGTGTAT
CATATGCCAAGTACGCCCCCTATTGACGTCAATGACGGTAAATGGCC
CGCCTGGCATTATGCCCAGTACATGACCTTATGGGACTTTCCTACTT
GGCAGTACATCTACGTATTAGTCATCGCTATTACCATGGTGATGCGG
TTTTGGCAGTACATCAATGGGCGTGGATAGCGGTTTGACTCACGGGG
ATTTCCAAGTCTCCACCCCATTGACGTCAATGGGAGTTTGTTTTGGC
ACCAAAATCAACGGGACTTTCCAAAATGTCGTAACAACTCCGCCCCA
TTGACGCAAATGGGCGGTAGGCGTGTACGGTGGGAGGTCTATATAAG
CAGAGCTCGTTTAGTGAACCGTcagatcgcctggagacgcACCGGTG
CCACCATGGCCCCTAAGAAGAAGCGGAAAGTCGGCAGCGGCAAGCGG
AACTATATCCTGGGCCTCGCCATCGGCATCACCAGCGTTGGCTACGG
CATCATCGACTACGAGACACGGGACGTGATCGATGCCGGCGTGCGGC
TGTTTAAAGAGGCCAACGTCGAGAACAACGAGGGCCGCAGATCTAAG
AGAGGCGCCAGACGGCTGAAGCGGAGAAGAAGGCACAGAATCCAGAG
AGTGAAGAAGCTGCTGTTCGACTACAACCTGCTGACCGACCACAGCG
AGCTGAGCGGCATCAATCCTTACGAGGCCAGAGTGAAGGGCCTGAGC
CAGAAGCTGAGCGAGGAAGAGTTTAGCGCCGCTCTGCTGCACCTGGC
CAAGAGAAGAGGCGTGCACAACGTGAACGAGGTGGAAGAGGACACCG
GCAACGAGCTGTCCACCAAAGAGCAGATCAGCCGGAACAGCAAGGCC
CTGGAAGAGAAATACGTGGCCGAACTGCAGCTGGAACGGCTGAAAAA
GGATGGCGAAGTGCGGGGCAGCATCAATCGGTTCAAGACCAGCGACT
ACGTGAAAGAAGCCAAACAGCTGCTGAAGGTGCAGAAGGCCTACCAC
CAGCTGGACCAGAGCTTCATCGACACCTACATCGACCTGCTGGAAAC
CCGGCGGACCTACTATGAAGGACCTGGCGAGGGAAGCCCCTTCGGCT
GGAAGGACATCAAAGAATGGTACGAGATGCTGATGGGCCACTGCACC
TACTTTCCCGAGGAACTGCGGAGCGTGAAGTACGCCTACAACGCCGA
CCTGTACAACGCCCTGAACGACCTGAACAACCTCGTGATCACCCGGG
ACGAGAACGAGAAGCTGGAATATTAGGAGAAGTTCCAGATCATCGAG
AACGTGTTCAAGCAGAAGAAGAAACCCACGCTGAAGCAGATCGCCAA
AGAGATCCTGGTCAACGAGGAAGATATCAAGGGCTACAGAGTGACCA
GCACCGGCAAGCCCGAGTTCACCAACCTGAAGGTGTACCACGACATC
AAGGATATCACAGCCCGGAAAGAGATTATTGAGAACGCCGAGCTGCT
GGACCAAATCGCCAAGATCCTGACCATCTACCAGAGCAGCGAGGACA
TCCAAGAGGAACTGACCAATCTGAACTCCGAGCTGACCCAAGAAGAG
ATCGAGCAGATCTCTAATCTGAAGGGGTACACCGGCACACACAACCT
GAGCCTGAAGGCCATCAACCTGATCCTGGACGAGCTGTGGCACACCA
ACGACAACCAGATTGCCATCTTCAACAGGCTGAAGCTGGTGCCCAAG
AAGGTGGACCTGTCTCAGCAGAAAGAAATCCCCACCACACTGGTGGA
CGACTTCATTCTGAGCCCCGTGGTCAAGAGGAGCTTCATCCAGAGCA
TCAAAGTGATCAACGCCATCATCAAGAAGTACGGGCTGCCCAACGAT
ATCATCATCGAGCTGGCCCGCGAGAAGAACTCCAAGGACGCCCAGAA
AATGATCAACGAGATGCAGAAGAGGAACCGCCAGACCAACGAGCGGA
TCGAGGAAATCATCCGGACCACCGGCAAAGAGAACGCCAAGTACCTG
ATCGAGAAGATCAAGCTGCACGACATGCAAGAGGGCAAGTGCCTGTA
CAGCCTGGAAGCCATTCCTCTGGAAGATCTGCTGAACAATCCCTTCA
ACTATGAGGTGGACCACATCATCCCCAGAAGCGTGTCCTTCGACAAC
AGCTTCAACAACAAGGTGCTCGTGAAGCAAGAGGAAGCCAGCAAGAA
GGGCAACAGAACCCCATTCCAGTACCTGAGCAGCAGCGACAGCAAGA
TCAGCTACGAAACCTTCAAGAAGCACATCCTGAATCTGGCCAAAGGC
AAGGGCAGAATCAGCAAGACCAAGAAAGAATACCTGCTCGAGGAACG
GGACATCAACAGATTCAGCGTGCAGAAAGACTTCATCAATCGGAACC
TGGTGGACACCAGATACGCCACCAGAGGCCTGATGAATCTGCTGCGG
AGCTACTTCAGAGTGAACAATCTGGACGTGAAAGTCAAGTCCATCAA
CGGCGGCTTCACCAGCTTTCTGCGGCGGAAGTGGAAGTTCAAGAAAG
AGCGGAACAAGGGCTATAAGCACCACGCCGAGGACGCCCTGATCATT
GCCAACGCCGATTTCATCTTCAAAGAGTGGAAGAAACTGGACAAGGC
CAAAAAAGTGATGGAAAACCAGATGTTCGAGGAAAAGCAGGCCGAGA
GCATGCCCGAGATCGAAACCGAGCAAGAGTACAAAGAGATTTTCATC
ACGCCCCACCAGATCAAGCACATTAAGGACTTCAAGGACTACAAGTA
CAGCCACCGCGTGGACAAGAAGCCCAATAGAGAGCTGATTAACGACA
CCCTGTACTCTACCCGGAAGGACGACAAGGGCAATACCCTGATCGTC
AACAACCTGAACGGCCTGTACGACAAGGACAACGACAAGCTCAAGAA
GCTGATCAACAAGAGCCCCGAGAAACTGCTGATGTACCACCACGATC
CTCAGACCTACCAGAAACTGAAGCTCATCATGGAACAGTACGGCGAC
GAGAAGAATCCCCTGTACAAGTACTACGAGGAAACCGGGAACTACCT
GACCAAGTACTCCAAAAAGGACAATGGGCCCGTGATCAAGAAGATTA
AGTATTAGGGCAACAAGCTGAATGCCCACCTGGACATCACCGACGAC
TACCCCAACAGCAGAAACAAGGTGGTCAAGCTGTCCCTGAAGCCTTA
CAGATTCGACGTGTACCTGGACAACGGCGTGTACAAGTTCGTGACCG
TGAAGAACCTGGACGTCATCAAAAAAGAAAACTACTAGGAAGTCAAC
AGCAAGTGCTATGAGGAAGCCAAGAAACTCAAGAAAATCAGCAACCA
GGCCGAGTTTATCGCCTCCTTCTACAACAACGATCTGATCAAGATCA
ACGGGGAGCTGTATAGAGTGATCGGCGTGAACAATGACCTGCTGAAC
CGGATCGAAGTGAACATGATCGACATCACCTACCGCGAGTACCTCGA
GAACATGAACGATAAGCGGCCTCCACGGATCATTAAGACAATCGCCA
GCAAGACGCAGAGCATTAAGAAGTACAGCACAGACATCCTGGGCAAC
CTGTACGAAGTGAAGTCTAAGAAGCACCCGCAGATTATCAAGAAAGG
CAGCGGCGCTCCCAAGAAAAAACGGAAGGTTGGCTCCGGAGAGGGCA
GAGGAAGTCTGCTAACATGCGGTGACGTCGAGGAGAATCCTGGCCCA
ATGGAGAGCGACGAGAGCGGCCTGCCCGCCATGGAGATCGAGTGCCG
CATCACCGGCACCCTGAACGGCGTGGAGTTCGAGCTGGTGGGCGGCG
GAGAGGGCACCCCCGAGCAGGGCCGCATGACCAACAAGATGAAGAGC
ACCAAAGGCGCCCTGACCTTCAGCCCCTACCTGCTGAGCCACGTGAT
GGGCTACGGCTTCTACCACTTCGGCACCTACCCCAGCGGCTACGAGA
ACCCCTTCCTGCACGCCATCAACAACGGCGGCTACACCAACACCCGC
ATCGAGAAGTACGAGGACGGCGGCGTGCTGCACGTGAGCTTCAGCTA
CCGCTACGAGGCCGGCCGCGTGATCGGCGACTTCAAGGTGATGGGCA
CCGGCTTCCCCGAGGACAGCGTGATCTTCACCGACAAGATCATCCGC
AGCAACGCCACCGTGGAGCACCTGCACCCCATGGGCGATAACGATCT
GGATGGCAGCTTCACCCGCACCTTCAGCCTGCGCGACGGCGGCTACT
ACAGCTCCGTGGTGGACAGCCACATGCACTTCAAGAGCGCCATCCAC
CCCAGCATCCTGCAGAACGGGGGCCCCATGTTCGCCTTCCGCCGCGT
GGAGGAGGATCACAGCAACACCGAGCTGGGCATCGTGGAGTACCAGC
ACGCCTTCAAGACCCCGGATGCAGATGCCGGTGAAGAATAAGAGCTC
GCTGATCAGCCTCGATAAGATACATTGATGAGTTTGGACAAACCACA
ACTAGAATGCAGTGAAAAAAATGCTTTATTTGTGAAATTTGTGATGC
TATTGCTTTATTTGTAACCATTATAAGCTGCAATAAACAAGTTTTAA
TTAAGGTCGGGCAGGAAGAGGGCCTATTTCCCATGATTCCTTCATAT
TTGCATATACGATACAAGGCTGTTAGAGAGATAATTAGAATTAATTT
GACTGTAAACACAAAGATATTAGTACAAAATACGTGACGTAGAAAGT
AATAATTTCTTGGGTAGTTTGCAGTTTTAAAATTATGTTTTAAAATG
GACTATCATATGCTTACCGTAACTTGAAAGTATTTCGATTTCTTGGC
TTTATATATCTTGTGGAAAGGACGAAACACCGAGGAGCACCAGGAAC
TTGCCAGTTTAAGTACTCTGTGCTGGAAACAGCACAGAATCTAGTTA
AACAAGGCAAAATGCCGTGTTTATCTCGTCAACTTGTTGGCGAGATT
TTTTCTAAAGCTTGAATTCAGCTGACGTGCCTCGGACCGCTAGGAAC
CCCTAGTGATGGAGTTGGCCACTCCCTCTCTGCGCGCTCGCTCGCTC
ACTGAGGCCGGGCGACCAAAGGTCGCCCGACGCCCGGGCTTTGCCCG
GGCGGCCTCAGTGAGCGAGCGAGCGCGCAG
Exemplary pITR-CMV.SaCas9T2AGFP.U6-hsa233Fw
sequence
(SEQ ID NO: 285)
CTGCGCGCTCGCTCGCTCACTGAGGCCGCCCGGGCGTCGGGCGACCT
TTGGTCGCCCGGCCTCAGTGAGCGAGCGAGCGCGCAGAGAGGGAGTG
GCCAACTCCATCACTAGGGGTTCCTGCGGCCGCACGCGTCTAGATCC
CATATATGGAGTTCCGCGTTACATAACTTACGGTAAATGGCCCGCCT
GGCTGACCGCCCAACGACCCCCGCCCATTGACGTCAATAATGACGTA
TGTTCCCATAGTAACGCCAATAGGGACTTTCCATTGACGTCAATGGG
TGGAGTATTTACGGTAAACTGCCCACTTGGCAGTACATCAAGTGTAT
CATATGCCAAGTACGCCCCCTATTGACGTCAATGACGGTAAATGGCC
CGCCTGGCATTATGCCCAGTACATGACCTTATGGGACTTTCCTACTT
GGCAGTACATCTACGTATTAGTCATCGCTATTACCATGGTGATGCGG
TTTTGGCAGTACATCAATGGGCGTGGATAGCGGTTTGACTCACGGGG
ATTTCCAAGTCTCCACCCCATTGACGTCAATGGGAGTTTGTTTTGGC
ACCAAAATCAACGGGACTTTCCAAAATGTCGTAACAACTCCGCCCCA
TTGACGCAAATGGGCGGTAGGCGTGTACGGTGGGAGGTCTATATAAG
CAGAGCTCGTTTAGTGAACCGTcagatcgcctggagacgcACCGGTG
CCACCATGGCCCCTAAGAAGAAGCGGAAAGTCGGCAGCGGCAAGCGG
AACTATATCCTGGGCCTCGACATCGGCATCACCAGCGTTGGCTACGG
CATCATCGACTACGAGACACGGGACGTGATCGATGCCGGCGTGCGGC
TGTTTAAAGAGGCCAACGTCGAGAACAACGAGGGCCGCAGATCTAAG
AGAGGCGCCAGACGGCTGAAGCGGAGAAGAAGGCACAGAATCCAGAG
AGTGAAGAAGCTGCTGTTCGACTACAACCTGCTGACCGACCACAGCG
AGCTGAGCGGCATCAATCCTTACGAGGCCAGAGTGAAGGGCCTGAGC
CAGAAGCTGAGCGAGGAAGAGTTTAGCGCCGCTCTGCTGCACCTGGC
CAAGAGAAGAGGCGTGCACAACGTGAACGAGGTGGAAGAGGACACCG
GCAACGAGCTGTCCACCAAAGAGCAGATCAGCCGGAACAGCAAGGCC
CTGGAAGAGAAATACGTGGCCGAACTGCAGCTGGAACGGCTGAAAAA
GGATGGCGAAGTGCGGGGCAGCATCAATCGGTTCAAGACCAGCGACT
ACGTGAAAGAAGCCAAACAGCTGCTGAAGGTGCAGAAGGCCTACCAC
CAGCTGGACCAGAGCTTCATCGACACCTACATCGACCTGCTGGAAAC
CCGGCGGACCTACTATGAAGGACCTGGCGAGGGAAGCCCCTTCGGCT
GGAAGGACATCAAAGAATGGTACGAGATGCTGATGGGCCACTGCACC
TACTTTCCCGAGGAACTGCGGAGCGTGAAGTACGCCTACAACGCCGA
CCTGTACAACGCCCTGAACGACCTGAACAACCTCGTGATCACCCGGG
ACGAGAACGAGAAGCTGGAATATTAGGAGAAGTTCCAGATCATCGAG
AACGTGTTCAAGCAGAAGAAGAAACCCACGCTGAAGCAGATCGCCAA
AGAGATCCTGGTCAACGAGGAAGATATCAAGGGCTACAGAGTGACCA
GCACCGGCAAGCCCGAGTTCACCAACCTGAAGGTGTACCACGACATC
AAGGATATCACAGCCCGGAAAGAGATTATTGAGAACGCCGAGCTGCT
GGACCAAATCGCCAAGATCCTGACCATCTACCAGAGCAGCGAGGACA
TCCAAGAGGAACTGACCAATCTGAACTCCGAGCTGACCCAAGAAGAG
ATCGAGCAGATCTCTAATCTGAAGGGGTACACCGGCACACACAACCT
GAGCCTGAAGGCCATCAACCTGATCCTGGACGAGCTGTGGCACACCA
ACGACAACCAGATTGCCATCTTCAACAGGCTGAAGCTGGTGCCCAAG
AAGGTGGACCTGTCTCAGCAGAAAGAAATCCCCACCACACTGGTGGA
CGACTTCATTCTGAGCCCCGTGGTCAAGAGGAGCTTCATCCAGAGCA
TCAAAGTGATCAACGCCATCATCAAGAAGTACGGGCTGCCCAACGAT
ATCATCATCGAGCTGGCCCGCGAGAAGAACTCCAAGGACGCCCAGAA
AATGATCAACGAGATGCAGAAGAGGAACCGCCAGACCAACGAGCGGA
TCGAGGAAATCATCCGGACCACCGGCAAAGAGAACGCCAAGTACCTG
ATCGAGAAGATCAAGCTGCACGACATGCAAGAGGGCAAGTGCCTGTA
CAGCCTGGAAGCCATTCCTCTGGAAGATCTGCTGAACAATCCCTTCA
ACTATGAGGTGGACCACATCATCCCCAGAAGCGTGTCCTTCGACAAC
AGCTTCAACAACAAGGTGCTCGTGAAGCAAGAGGAAAACAGCAAGAA
GGGCAACAGAACCCCATTCCAGTACCTGAGCAGCAGCGACAGCAAGA
TCAGCTACGAAACCTTCAAGAAGCACATCCTGAATCTGGCCAAAGGC
AAGGGCAGAATCAGCAAGACCAAGAAAGAATACCTGCTCGAGGAACG
GGACATCAACAGATTCAGCGTGCAGAAAGACTTCATCAATCGGAACC
TGGTGGACACCAGATACGCCACCAGAGGCCTGATGAATCTGCTGCGG
AGCTACTTCAGAGTGAACAATCTGGACGTGAAAGTCAAGTCCATCAA
CGGCGGCTTCACCAGCTTTCTGCGGCGGAAGTGGAAGTTCAAGAAAG
AGCGGAACAAGGGCTATAAGCACCACGCCGAGGACGCCCTGATCATT
GCCAACGCCGATTTCATCTTCAAAGAGTGGAAGAAACTGGACAAGGC
CAAAAAAGTGATGGAAAACCAGATGTTCGAGGAAAAGCAGGCCGAGA
GCATGCCCGAGATCGAAACCGAGCAAGAGTACAAAGAGATTTTCATC
ACGCCCCACCAGATCAAGCACATTAAGGACTTCAAGGACTACAAGTA
CAGCCACCGCGTGGACAAGAAGCCCAATAGAGAGCTGATTAACGACA
CCCTGTACTCTACCCGGAAGGACGACAAGGGCAATACCCTGATCGTC
AACAACCTGAACGGCCTGTACGACAAGGACAACGACAAGCTCAAGAA
GCTGATCAACAAGAGCCCCGAGAAACTGCTGATGTACCACCACGATC
CTCAGACCTACCAGAAACTGAAGCTCATCATGGAACAGTACGGCGAC
GAGAAGAATCCCCTGTACAAGTACTACGAGGAAACCGGGAACTACCT
GACCAAGTACTCCAAAAAGGACAATGGGCCCGTGATCAAGAAGATTA
AGTATTAGGGCAACAAGCTGAATGCCCACCTGGACATCACCGACGAC
TACCCCAACAGCAGAAACAAGGTGGTCAAGCTGTCCCTGAAGCCTTA
CAGATTCGACGTGTACCTGGACAACGGCGTGTACAAGTTCGTGACCG
TGAAGAACCTGGACGTCATCAAAAAAGAAAACTACTAGGAAGTCAAC
AGCAAGTGCTATGAGGAAGCCAAGAAACTCAAGAAAATCAGCAACCA
GGCCGAGTTTATCGCCTCCTTCTACAACAACGATCTGATCAAGATCA
ACGGGGAGCTGTATAGAGTGATCGGCGTGAACAATGACCTGCTGAAC
CGGATCGAAGTGAACATGATCGACATCACCTACCGCGAGTACCTCGA
GAACATGAACGATAAGCGGCCTCCACGGATCATTAAGACAATCGCCA
GCAAGACGCAGAGCATTAAGAAGTACAGCACAGACATCCTGGGCAAC
CTGTACGAAGTGAAGTCTAAGAAGCACCCGCAGATTATCAAGAAAGG
CAGCGGCGCTCCCAAGAAAAAACGGAAGGTTGGCTCCGGAGAGGGCA
GAGGAAGTCTGCTAACATGCGGTGACGTCGAGGAGAATCCTGGCCCA
ATGGAGAGCGACGAGAGCGGCCTGCCCGCCATGGAGATCGAGTGCCG
CATCACCGGCACCCTGAACGGCGTGGAGTTCGAGCTGGTGGGCGGCG
GAGAGGGCACCCCCGAGCAGGGCCGCATGACCAACAAGATGAAGAGC
ACCAAAGGCGCCCTGACCTTCAGCCCCTACCTGCTGAGCCACGTGAT
GGGCTACGGCTTCTACCACTTCGGCACCTACCCCAGCGGCTACGAGA
ACCCCTTCCTGCACGCCATCAACAACGGCGGCTACACCAACACCCGC
ATCGAGAAGTACGAGGACGGCGGCGTGCTGCACGTGAGCTTCAGCTA
CCGCTACGAGGCCGGCCGCGTGATCGGCGACTTCAAGGTGATGGGCA
CCGGCTTCCCCGAGGACAGCGTGATCTTCACCGACAAGATCATCCGC
AGCAACGCCACCGTGGAGCACCTGCACCCCATGGGCGATAACGATCT
GGATGGCAGCTTCACCCGCACCTTCAGCCTGCGCGACGGCGGCTACT
ACAGCTCCGTGGTGGACAGCCACATGCACTTCAAGAGCGCCATCCAC
CCCAGCATCCTGCAGAACGGGGGCCCCATGTTCGCCTTCCGCCGCGT
GGAGGAGGATCACAGCAACACCGAGCTGGGCATCGTGGAGTACCAGC
ACGCCTTCAAGACCCCGGATGCAGATGCCGGTGAAGAATAAGAGCTC
GCTGATCAGCCTCGATAAGATACATTGATGAGTTTGGACAAACCACA
ACTAGAATGCAGTGAAAAAAATGCTTTATTTGTGAAATTTGTGATGC
TATTGCTTTATTTGTAACCATTATAAGCTGCAATAAACAAGTTTTAA
TTAAGGTCGGGCAGGAAGAGGGCCTATTTCCCATGATTCCTTCATAT
TTGCATATACGATACAAGGCTGTTAGAGAGATAATTAGAATTAATTT
GACTGTAAACACAAAGATATTAGTACAAAATACGTGACGTAGAAAGT
AATAATTTCTTGGGTAGTTTGCAGTTTTAAAATTATGTTTTAAAATG
GACTATCATATGCTTACCGTAACTTGAAAGTATTTCGATTTCTTGGC
TTTATATATCTTGTGGAAAGGACGAAACACCGGCGCTACCGCCGCCT
GCAGAAGTTTAAGTACTCTGTGCTGGAAACAGCACAGAATCTAGTTA
AACAAGGCAAAATGCCGTGTTTATCTCGTCAACTTGTTGGCGAGATT
TTTTCtaAAGCTTGAATTCAGCTGACGTGCCTCGGACCGCTAGGAAC
CCCTAGTGATGGAGTTGGCCACTCCCTCTCTGCGCGCTCGCTCGCTC
ACTGAGGCCGGGCGACCAAAGGTCGCCCGACGCCCGGGCTTTGCCCG
GGCGGCCTCAGTGAGCGAGCGAGCGCGCAG
Exemplary pITR-CMV.SaCas9T2AGFP.U6-hsa447Fw
sequence
(SEQ ID NO: 286)
CTGCGCGCTCGCTCGCTCACTGAGGCCGCCCGGGCGTCGGGCGACCT
TTGGTCGCCCGGCCTCAGTGAGCGAGCGAGCGCGCAGAGAGGGAGTG
GCCAACTCCATCACTAGGGGTTCCTGCGGCCGCACGCGTCTAGATCC
CATATATGGAGTTCCGCGTTACATAACTTACGGTAAATGGCCCGCCT
GGCTGACCGCCCAACGACCCCCGCCCATTGACGTCAATAATGACGTA
TGTTCCCATAGTAACGCCAATAGGGACTTTCCATTGACGTCAATGGG
TGGAGTATTTACGGTAAACTGCCCACTTGGCAGTACATCAAGTGTAT
CATATGCCAAGTACGCCCCCTATTGACGTCAATGACGGTAAATGGCC
CGCCTGGCATTATGCCCAGTACATGACCTTATGGGACTTTCCTACTT
GGCAGTACATCTACGTATTAGTCATCGCTATTACCATGGTGATGCGG
TTTTGGCAGTACATCAATGGGCGTGGATAGCGGTTTGACTCACGGGG
ATTTCCAAGTCTCCACCCCATTGACGTCAATGGGAGTTTGTTTTGGC
ACCAAAATCAACGGGACTTTCCAAAATGTCGTAACAACTCCGCCCCA
TTGACGCAAATGGGCGGTAGGCGTGTACGGTGGGAGGTCTATATAAG
CAGAGCTCGTTTAGTGAACCGTcagatcgcctggagacgcACCGGTG
CCACCATGGCCCCTAAGAAGAAGCGGAAAGTCGGCAGCGGCAAGCGG
AACTATATCCTGGGCCTCGACATCGGCATCACCAGCGTTGGCTACGG
CATCATCGACTACGAGACACGGGACGTGATCGATGCCGGCGTGCGGC
TGTTTAAAGAGGCCAACGTCGAGAACAACGAGGGCCGCAGATCTAAG
AGAGGCGCCAGACGGCTGAAGCGGAGAAGAAGGCACAGAATCCAGAG
AGTGAAGAAGCTGCTGTTCGACTACAACCTGCTGACCGACCACAGCG
AGCTGAGCGGCATCAATCCTTACGAGGCCAGAGTGAAGGGCCTGAGC
CAGAAGCTGAGCGAGGAAGAGTTTAGCGCCGCTCTGCTGCACCTGGC
CAAGAGAAGAGGCGTGCACAACGTGAACGAGGTGGAAGAGGACACCG
GCAACGAGCTGTCCACCAAAGAGCAGATCAGCCGGAACAGCAAGGCC
CTGGAAGAGAAATACGTGGCCGAACTGCAGCTGGAACGGCTGAAAAA
GGATGGCGAAGTGCGGGGCAGCATCAATCGGTTCAAGACCAGCGACT
ACGTGAAAGAAGCCAAACAGCTGCTGAAGGTGCAGAAGGCCTACCAC
CAGCTGGACCAGAGCTTCATCGACACCTACATCGACCTGCTGGAAAC
CCGGCGGACCTACTATGAAGGACCTGGCGAGGGAAGCCCCTTCGGCT
GGAAGGACATCAAAGAATGGTACGAGATGCTGATGGGCCACTGCACC
TACTTTCCCGAGGAACTGCGGAGCGTGAAGTACGCCTACAACGCCGA
CCTGTACAACGCCCTGAACGACCTGAACAACCTCGTGATCACCCGGG
ACGAGAACGAGAAGCTGGAATATTAGGAGAAGTTCCAGATCATCGAG
AACGTGTTCAAGCAGAAGAAGAAACCCACGCTGAAGCAGATCGCCAA
AGAGATCCTGGTCAACGAGGAAGATATCAAGGGCTACAGAGTGACCA
GCACCGGCAAGCCCGAGTTCACCAACCTGAAGGTGTACCACGACATC
AAGGATATCACAGCCCGGAAAGAGATTATTGAGAACGCCGAGCTGCT
GGACCAAATCGCCAAGATCCTGACCATCTACCAGAGCAGCGAGGACA
TCCAAGAGGAACTGACCAATCTGAACTCCGAGCTGACCCAAGAAGAG
ATCGAGCAGATCTCTAATCTGAAGGGGTACACCGGCACACACAACCT
GAGCCTGAAGGCCATCAACCTGATCCTGGACGAGCTGTGGCACACCA
ACGACAACCAGATTGCCATCTTCAACAGGCTGAAGCTGGTGCCCAAG
AAGGTGGACCTGTCTCAGCAGAAAGAAATCCCCACCACACTGGTGGA
CGACTTCATTCTGAGCCCCGTGGTCAAGAGGAGCTTCATCCAGAGCA
TCAAAGTGATCAACGCCATCATCAAGAAGTACGGGCTGCCCAACGAT
ATCATCATCGAGCTGGCCCGCGAGAAGAACTCCAAGGACGCCCAGAA
AATGATCAACGAGATGCAGAAGAGGAACCGCCAGACCAACGAGCGGA
TCGAGGAAATCATCCGGACCACCGGCAAAGAGAACGCCAAGTACCTG
ATCGAGAAGATCAAGCTGCACGACATGCAAGAGGGCAAGTGCCTGTA
CAGCCTGGAAGCCATTCCTCTGGAAGATCTGCTGAACAATCCCTTCA
ACTATGAGGTGGACCACATCATCCCCAGAAGCGTGTCCTTCGACAAC
AGCTTCAACAACAAGGTGCTCGTGAAGCAAGAGGAAAACAGCAAGAA
GGGCAACAGAACCCCATTCCAGTACCTGAGCAGCAGCGACAGCAAGA
TCAGCTACGAAACCTTCAAGAAGCACATCCTGAATCTGGCCAAAGGC
AAGGGCAGAATCAGCAAGACCAAGAAAGAATACCTGCTCGAGGAACG
GGACATCAACAGATTCAGCGTGCAGAAAGACTTCATCAATCGGAACC
TGGTGGACACCAGATACGCCACCAGAGGCCTGATGAATCTGCTGCGG
AGCTACTTCAGAGTGAACAATCTGGACGTGAAAGTCAAGTCCATCAA
CGGCGGCTTCACCAGCTTTCTGCGGCGGAAGTGGAAGTTCAAGAAAG
AGCGGAACAAGGGCTATAAGCACCACGCCGAGGACGCCCTGATCATT
GCCAACGCCGATTTCATCTTCAAAGAGTGGAAGAAACTGGACAAGGC
CAAAAAAGTGATGGAAAACCAGATGTTCGAGGAAAAGCAGGCCGAGA
GCATGCCCGAGATCGAAACCGAGCAAGAGTACAAAGAGATTTTCATC
ACGCCCCACCAGATCAAGCACATTAAGGACTTCAAGGACTACAAGTA
CAGCCACCGCGTGGACAAGAAGCCCAATAGAGAGCTGATTAACGACA
CCCTGTACTCTACCCGGAAGGACGACAAGGGCAATACCCTGATCGTC
AACAACCTGAACGGCCTGTACGACAAGGACAACGACAAGCTCAAGAA
GCTGATCAACAAGAGCCCCGAGAAACTGCTGATGTACCACCACGATC
CTCAGACCTACCAGAAACTGAAGCTCATCATGGAACAGTACGGCGAC
GAGAAGAATCCCCTGTACAAGTACTACGAGGAAACCGGGAACTACCT
GACCAAGTACTCCAAAAAGGACAATGGGCCCGTGATCAAGAAGATTA
AGTATTAGGGCAACAAGCTGAATGCCCACCTGGACATCACCGACGAC
TACCCCAACAGCAGAAACAAGGTGGTCAAGCTGTCCCTGAAGCCTTA
CAGATTCGACGTGTACCTGGACAACGGCGTGTACAAGTTCGTGACCG
TGAAGAACCTGGACGTCATCAAAAAAGAAAACTACTAGGAAGTCAAC
AGCAAGTGCTATGAGGAAGCCAAGAAACTCAAGAAAATCAGCAACCA
GGCCGAGTTTATCGCCTCCTTCTACAACAACGATCTGATCAAGATCA
ACGGGGAGCTGTATAGAGTGATCGGCGTGAACAATGACCTGCTGAAC
CGGATCGAAGTGAACATGATCGACATCACCTACCGCGAGTACCTCGA
GAACATGAACGATAAGCGGCCTCCACGGATCATTAAGACAATCGCCA
GCAAGACGCAGAGCATTAAGAAGTACAGCACAGACATCCTGGGCAAC
CTGTACGAAGTGAAGTCTAAGAAGCACCCGCAGATTATCAAGAAAGG
CAGCGGCGCTCCCAAGAAAAAACGGAAGGTTGGCTCCGGAGAGGGCA
GAGGAAGTCTGCTAACATGCGGTGACGTCGAGGAGAATCCTGGCCCA
ATGGAGAGCGACGAGAGCGGCCTGCCCGCCATGGAGATCGAGTGCCG
CATCACCGGCACCCTGAACGGCGTGGAGTTCGAGCTGGTGGGCGGCG
GAGAGGGCACCCCCGAGCAGGGCCGCATGACCAACAAGATGAAGAGC
ACCAAAGGCGCCCTGACCTTCAGCCCCTACCTGCTGAGCCACGTGAT
GGGCTACGGCTTCTACCACTTCGGCACCTACCCCAGCGGCTACGAGA
ACCCCTTCCTGCACGCCATCAACAACGGCGGCTACACCAACACCCGC
ATCGAGAAGTACGAGGACGGCGGCGTGCTGCACGTGAGCTTCAGCTA
CCGCTACGAGGCCGGCCGCGTGATCGGCGACTTCAAGGTGATGGGCA
CCGGCTTCCCCGAGGACAGCGTGATCTTCACCGACAAGATCATCCGC
AGCAACGCCACCGTGGAGCACCTGCACCCCATGGGCGATAACGATCT
GGATGGCAGCTTCACCCGCACCTTCAGCCTGCGCGACGGCGGCTACT
ACAGCTCCGTGGTGGACAGCCACATGCACTTCAAGAGCGCCATCCAC
CCCAGCATCCTGCAGAACGGGGGCCCCATGTTCGCCTTCCGCCGCGT
GGAGGAGGATCACAGCAACACCGAGCTGGGCATCGTGGAGTACCAGC
ACGCCTTCAAGACCCCGGATGCAGATGCCGGTGAAGAATAAGAGCTC
GCTGATCAGCCTCGATAAGATACATTGATGAGTTTGGACAAACCACA
ACTAGAATGCAGTGAAAAAAATGCTTTATTTGTGAAATTTGTGATGC
TATTGCTTTATTTGTAACCATTATAAGCTGCAATAAACAAGTTTTAA
TTAAGGTCGGGCAGGAAGAGGGCCTATTTCCCATGATTCCTTCATAT
TTGCATATACGATACAAGGCTGTTAGAGAGATAATTAGAATTAATTT
GACTGTAAACACAAAGATATTAGTACAAAATACGTGACGTAGAAAGT
AATAATTTCTTGGGTAGTTTGCAGTTTTAAAATTATGTTTTAAAATG
GACTATCATATGCTTACCGTAACTTGAAAGTATTTCGATTTCTTGGC
TTTATATATCTTGTGGAAAGGACGAAACACCGTTTCGGCTTGGAGTA
CATCGTGTTTAAGTACTCTGTGCTGGAAACAGCACAGAATCTACTTA
AACAAGGCAAAATGCCGTGTTTATCTCGTCAACTTGTTGGCGAGATT
TTTTCTAAAGCTTGAATTCAGCTGACGTGCCTCGGACCGCTAGGAAC
CCCTAGTGATGGAGTTGGCCACTCCCTCTCTGCGCGCTCGCTCGCTC
ACTGAGGCCGGGCGACCAAAGGTCGCCCGACGCCCGGGCTTTGCCCG
GGCGGCCTCAGTGAGCGAGCGAGCGCGCAG
Exemplary pITR-CMV.SaCas9T2AGFP.U6-hsa482Fw
sequence
(SEQ ID NO: 287)
CTGCGCGCTCGCTCGCTCACTGAGGCCGCCCGGGCGTCGGGCGACCT
TTGGTCGCCCGGCCTCAGTGAGCGAGCGAGCGCGCAGAGAGGGAGTG
GCCAACTCCATCACTAGGGGTTCCTGCGGCCGCACGCGTCTAGATCC
CATATATGGAGTTCCGCGTTACATAACTTACGGTAAATGGCCCGCCT
GGCTGACCGCCCAACGACCCCCGCCCATTGACGTCAATAATGACGTA
TGTTCCCATAGTAACGCCAATAGGGACTTTCCATTGACGTCAATGGG
TGGAGTATTTACGGTAAACTGCCCACTTGGCAGTACATCAAGTGTAT
CATATGCCAAGTACGCCCCCTATTGACGTCAATGACGGTAAATGGCC
CGCCTGGCATTATGCCCAGTACATGACCTTATGGGACTTTCCTACTT
GGCAGTACATCTACGTATTAGTCATCGCTATTACCATGGTGATGCGG
TTTTGGCAGTACATCAATGGGCGTGGATAGCGGTTTGACTCACGGGG
ATTTCCAAGTCTCCACCCCATTGACGTCAATGGGAGTTTGTTTTGGC
ACCAAAATCAACGGGACTTTCCAAAATGTCGTAACAACTCCGCCCCA
TTGACGCAAATGGGCGGTAGGCGTGTACGGTGGGAGGTCTATATAAG
CAGAGCTCGTTTAGTGAACCGTcagatcgcctggagacgcACCGGTG
CCACCATGGCCCCTAAGAAGAAGCGGAAAGTCGGCAGCGGCAAGCGG
AACTATATCCTGGGCCTCGACATCGGCATCACCAGCGTTGGCTACGG
CATCATCGACTACGAGACACGGGACGTGATCGATGCCGGCGTGCGGC
TGTTTAAAGAGGCCAACGTCGAGAACAACGAGGGCCGCAGATCTAAG
AGAGGCGCCAGACGGCTGAAGCGGAGAAGAAGGCACAGAATCCAGAG
AGTGAAGAAGCTGCTGTTCGACTACAACCTGCTGACCGACCACAGCG
AGCTGAGCGGCATCAATCCTTACGAGGCCAGAGTGAAGGGCCTGAGC
CAGAAGCTGAGCGAGGAAGAGTTTAGCGCCGCTCTGCTGCACCTGGC
CAAGAGAAGAGGCGTGCACAACGTGAACGAGGTGGAAGAGGACACCG
GCAACGAGCTGTCCACCAAAGAGCAGATCAGCCGGAACAGCAAGGCC
CTGGAAGAGAAATACGTGGCCGAACTGCAGCTGGAACGGCTGAAAAA
GGATGGCGAAGTGCGGGGCAGCATCAATCGGTTCAAGACCAGCGACT
ACGTGAAAGAAGCCAAACAGCTGCTGAAGGTGCAGAAGGCCTACCAC
CAGCTGGACCAGAGCTTCATCGACACCTACATCGACCTGCTGGAAAC
CCGGCGGACCTACTATGAAGGACCTGGCGAGGGAAGCCCCTTCGGCT
GGAAGGACATCAAAGAATGGTACGAGATGCTGATGGGCCACTGCACC
TACTTTCCCGAGGAACTGCGGAGCGTGAAGTACGCCTACAACGCCGA
CCTGTACAACGCCCTGAACGACCTGAACAACCTCGTGATCACCCGGG
ACGAGAACGAGAAGCTGGAATATTAGGAGAAGTTCCAGATCATCGAG
AACGTGTTCAAGCAGAAGAAGAAACCCACGCTGAAGCAGATCGCCAA
AGAGATCCTGGTCAACGAGGAAGATATCAAGGGCTACAGAGTGACCA
GCACCGGCAAGCCCGAGTTCACCAACCTGAAGGTGTACCACGACATC
AAGGATATCACAGCCCGGAAAGAGATTATTGAGAACGCCGAGCTGCT
GGACCAAATCGCCAAGATCCTGACCATCTACCAGAGCAGCGAGGACA
TCCAAGAGGAACTGACCAATCTGAACTCCGAGCTGACCCAAGAAGAG
ATCGAGCAGATCTCTAATCTGAAGGGGTACACCGGCACACACAACCT
GAGCCTGAAGGCCATCAACCTGATCCTGGACGAGCTGTGGCACACCA
ACGACAACCAGATTGCCATCTTCAACAGGCTGAAGCTGGTGCCCAAG
AAGGTGGACCTGTCTCAGCAGAAAGAAATCCCCACCACACTGGTGGA
CGACTTCATTCTGAGCCCCGTGGTCAAGAGGAGCTTCATCCAGAGCA
TCAAAGTGATCAACGCCATCATCAAGAAGTACGGGCTGCCCAACGAT
ATCATCATCGAGCTGGCCCGCGAGAAGAACTCCAAGGACGCCCAGAA
AATGATCAACGAGATGCAGAAGAGGAACCGCCAGACCAACGAGCGGA
TCGAGGAAATCATCCGGACCACCGGCAAAGAGAACGCCAAGTACCTG
ATCGAGAAGATCAAGCTGCACGACATGCAAGAGGGCAAGTGCCTGTA
CAGCCTGGAAGCCATTCCTCTGGAAGATCTGCTGAACAATCCCTTCA
ACTATGAGGTGGACCACATCATCCCCAGAAGCGTGTCCTTCGACAAC
AGCTTCAACAACAAGGTGCTCGTGAAGCAAGAGGAAAACAGCAAGAA
GGGCAACAGAACCCCATTCCAGTACCTGAGCAGCAGCGACAGCAAGA
TCAGCTACGAAACCTTCAAGAAGCACATCCTGAATCTGGCCAAAGGC
AAGGGCAGAATCAGCAAGACCAAGAAAGAATACCTGCTCGAGGAACG
GGACATCAACAGATTCAGCGTGCAGAAAGACTTCATCAATCGGAACC
TGGTGGACACCAGATACGCCACCAGAGGCCTGATGAATCTGCTGCGG
AGCTACTTCAGAGTGAACAATCTGGACGTGAAAGTCAAGTCCATCAA
CGGCGGCTTCACCAGCTTTCTGCGGCGGAAGTGGAAGTTCAAGAAAG
AGCGGAACAAGGGCTATAAGCACCACGCCGAGGACGCCCTGATCATT
GCCAACGCCGATTTCATCTTCAAAGAGTGGAAGAAACTGGACAAGGC
CAAAAAAGTGATGGAAAACCAGATGTTCGAGGAAAAGCAGGCCGAGA
GCATGCCCGAGATCGAAACCGAGCAAGAGTACAAAGAGATTTTCATC
ACGCCCCACCAGATCAAGCACATTAAGGACTTCAAGGACTACAAGTA
CAGCCACCGCGTGGACAAGAAGCCCAATAGAGAGCTGATTAACGACA
CCCTGTACTCTACCCGGAAGGACGACAAGGGCAATACCCTGATCGTC
AACAACCTGAACGGCCTGTACGACAAGGACAACGACAAGCTCAAGAA
GCTGATCAACAAGAGCCCCGAGAAACTGCTGATGTACCACCACGATC
CTCAGACCTACCAGAAACTGAAGCTCATCATGGAACAGTACGGCGAC
GAGAAGAATCCCCTGTACAAGTACTACGAGGAAACCGGGAACTACCT
GACCAAGTACTCCAAAAAGGACAATGGGCCCGTGATCAAGAAGATTA
AGTATTAGGGCAACAAGCTGAATGCCCACCTGGACATCACCGACGAC
TACCCCAACAGCAGAAACAAGGTGGTCAAGCTGTCCCTGAAGCCTTA
CAGATTCGACGTGTACCTGGACAACGGCGTGTACAAGTTCGTGACCG
TGAAGAACCTGGACGTCATCAAAAAAGAAAACTACTAGGAAGTCAAC
AGCAAGTGCTATGAGGAAGCCAAGAAACTCAAGAAAATCAGCAACCA
GGCCGAGTTTATCGCCTCCTTCTACAACAACGATCTGATCAAGATCA
ACGGGGAGCTGTATAGAGTGATCGGCGTGAACAATGACCTGCTGAAC
CGGATCGAAGTGAACATGATCGACATCACCTACCGCGAGTACCTCGA
GAACATGAACGATAAGCGGCCTCCACGGATCATTAAGACAATCGCCA
GCAAGACGCAGAGCATTAAGAAGTACAGCACAGACATCCTGGGCAAC
CTGTACGAAGTGAAGTCTAAGAAGCACCCGCAGATTATCAAGAAAGG
CAGCGGCGCTCCCAAGAAAAAACGGAAGGTTGGCTCCGGAGAGGGCA
GAGGAAGTCTGCTAACATGCGGTGACGTCGAGGAGAATCCTGGCCCA
ATGGAGAGCGACGAGAGCGGCCTGCCCGCCATGGAGATCGAGTGCCG
CATCACCGGCACCCTGAACGGCGTGGAGTTCGAGCTGGTGGGCGGCG
GAGAGGGCACCCCCGAGCAGGGCCGCATGACCAACAAGATGAAGAGC
ACCAAAGGCGCCCTGACCTTCAGCCCCTACCTGCTGAGCCACGTGAT
GGGCTACGGCTTCTACCACTTCGGCACCTACCCCAGCGGCTACGAGA
ACCCCTTCCTGCACGCCATCAACAACGGCGGCTACACCAACACCCGC
ATCGAGAAGTACGAGGACGGCGGCGTGCTGCACGTGAGCTTCAGCTA
CCGCTACGAGGCCGGCCGCGTGATCGGCGACTTCAAGGTGATGGGCA
CCGGCTTCCCCGAGGACAGCGTGATCTTCACCGACAAGATCATCCGC
AGCAACGCCACCGTGGAGCACCTGCACCCCATGGGCGATAACGATCT
GGATGGCAGCTTCACCCGCACCTTCAGCCTGCGCGACGGCGGCTACT
ACAGCTCCGTGGTGGACAGCCACATGCACTTCAAGAGCGCCATCCAC
CCCAGCATCCTGCAGAACGGGGGCCCCATGTTCGCCTTCCGCCGCGT
GGAGGAGGATCACAGCAACACCGAGCTGGGCATCGTGGAGTACCAGC
ACGCCTTCAAGACCCCGGATGCAGATGCCGGTGAAGAATAAGAGCTC
GCTGATCAGCCTCGATAAGATACATTGATGAGTTTGGACAAACCACA
ACTAGAATGCAGTGAAAAAAATGCTTTATTTGTGAAATTTGTGATGC
TATTGCTTTATTTGTAACCATTATAAGCTGCAATAAACAAGTTTTAA
TTAAGGTCGGGCAGGAAGAGGGCCTATTTCCCATGATTCCTTCATAT
TTGCATATACGATACAAGGCTGTTAGAGAGATAATTAGAATTAATTT
GACTGTAAACACAAAGATATTAGTACAAAATACGTGACGTAGAAAGT
AATAATTTCTTGGGTAGTTTGCAGTTTTAAAATTATGTTTTAAAATG
GACTATCATATGCTTACCGTAACTTGAAAGTATTTCGATTTCTTGGC
TTTATATATCTTGTGGAAAGGACGAAACACCGCCGGATGCTGCTGCC
GCTACCGTTTAAGTACTCTGTGCTGGAAACAGCACAGAATCTACTTA
AACAAGGCAAAATGCCGTGTTTATCTCGTCAACTTGTTGGCGAGATT
TTTTCTAAAGCTTGAATTCAGCTGACGTGCCTCGGACCGCTAGGAAC
CCCTAGTGATGGAGTTGGCCACTCCCTCTCTGCGCGCTCGCTCGCTC
ACTGAGGCCGGGCGACCAAAGGTCGCCCGACGCCCGGGCTTTGCCCG
GGCGGCCTCAGTGAGCGAGCGAGCGCGCAG
Exemplary pITR-CMV.SaCas9T2AGFP.U6-hsa490Fw
sequence
(SEQ ID NO: 288)
CTGCGCGCTCGCTCGCTCACTGAGGCCGCCCGGGCGTCGGGCGACCT
TTGGTCGCCCGGCCTCAGTGAGCGAGCGAGCGCGCAGAGAGGGAGTG
GCCAACTCCATCACTAGGGGTTCCTGCGGCCGCACGCGTCTAGATCC
CATATATGGAGTTCCGCGTTACATAACTTACGGTAAATGGCCCGCCT
GGCTGACCGCCCAACGACCCCCGCCCATTGACGTCAATAATGACGTA
TGTTCCCATAGTAACGCCAATAGGGACTTTCCATTGACGTCAATGGG
TGGAGTATTTACGGTAAACTGCCCACTTGGCAGTACATCAAGTGTAT
CATATGCCAAGTACGCCCCCTATTGACGTCAATGACGGTAAATGGCC
CGCCTGGCATTATGCCCAGTACATGACCTTATGGGACTTTCCTACTT
GGCAGTACATCTACGTATTAGTCATCGCTATTACCATGGTGATGCGG
TTTTGGCAGTACATCAATGGGCGTGGATAGCGGTTTGACTCACGGGG
ATTTCCAAGTCTCCACCCCATTGACGTCAATGGGAGTTTGTTTTGGC
ACCAAAATCAACGGGACTTTCCAAAATGTCGTAACAACTCCGCCCCA
TTGACGCAAATGGGCGGTAGGCGTGTACGGTGGGAGGTCTATATAAG
CAGAGCTCGTTTAGTGAACCGTcagatcgcctggagacgcACCGGTG
CCACCATGGCCCCTAAGAAGAAGCGGAAAGTCGGCAGCGGCAAGCGG
AACTATATCCTGGGCCTCGACATCGGCATCACCAGCGTTGGCTACGG
CATCATCGACTACGAGACACGGGACGTGATCGATGCCGGCGTGCGGC
TGTTTAAAGAGGCCAACGTCGAGAACAACGAGGGCCGCAGATCTAAG
AGAGGCGCCAGACGGCTGAAGCGGAGAAGAAGGCACAGAATCCAGAG
AGTGAAGAAGCTGCTGTTCGACTACAACCTGCTGACCGACCACAGCG
AGCTGAGCGGCATCAATCCTTACGAGGCCAGAGTGAAGGGCCTGAGC
CAGAAGCTGAGCGAGGAAGAGTTTAGCGCCGCTCTGCTGCACCTGGC
CAAGAGAAGAGGCGTGCACAACGTGAACGAGGTGGAAGAGGACACCG
GCAACGAGCTGTCCACCAAAGAGCAGATCAGCCGGAACAGCAAGGCC
CTGGAAGAGAAATACGTGGCCGAACTGCAGCTGGAACGGCTGAAAAA
GGATGGCGAAGTGCGGGGCAGCATCAATCGGTTCAAGACCAGCGACT
ACGTGAAAGAAGCCAAACAGCTGCTGAAGGTGCAGAAGGCCTACCAC
CAGCTGGACCAGAGCTTCATCGACACCTACATCGACCTGCTGGAAAC
CCGGCGGACCTACTATGAAGGACCTGGCGAGGGAAGCCCCTTCGGCT
GGAAGGACATCAAAGAATGGTACGAGATGCTGATGGGCCACTGCACC
TACTTTCCCGAGGAACTGCGGAGCGTGAAGTACGCCTACAACGCCGA
CCTGTACAACGCCCTGAACGACCTGAACAACCTCGTGATCACCCGGG
ACGAGAACGAGAAGCTGGAATATTAGGAGAAGTTCCAGATCATCGAG
AACGTGTTCAAGCAGAAGAAGAAACCCACGCTGAAGCAGATCGCCAA
AGAGATCCTGGTCAACGAGGAAGATATCAAGGGCTACAGAGTGACCA
GCACCGGCAAGCCCGAGTTCACCAACCTGAAGGTGTACCACGACATC
AAGGATATCACAGCCCGGAAAGAGATTATTGAGAACGCCGAGCTGCT
GGACCAAATCGCCAAGATCCTGACCATCTACCAGAGCAGCGAGGACA
TCCAAGAGGAACTGACCAATCTGAACTCCGAGCTGACCCAAGAAGAG
ATCGAGCAGATCTCTAATCTGAAGGGGTACACCGGCACACACAACCT
GAGCCTGAAGGCCATCAACCTGATCCTGGACGAGCTGTGGCACACCA
ACGACAACCAGATTGCCATCTTCAACAGGCTGAAGCTGGTGCCCAAG
AAGGTGGACCTGTCTCAGCAGAAAGAAATCCCCACCACACTGGTGGA
CGACTTCATTCTGAGCCCCGTGGTCAAGAGGAGCTTCATCCAGAGCA
TCAAAGTGATCAACGCCATCATCAAGAAGTACGGGCTGCCCAACGAT
ATCATCATCGAGCTGGCCCGCGAGAAGAACTCCAAGGACGCCCAGAA
AATGATCAACGAGATGCAGAAGAGGAACCGCCAGACCAACGAGCGGA
TCGAGGAAATCATCCGGACCACCGGCAAAGAGAACGCCAAGTACCTG
ATCGAGAAGATCAAGCTGCACGACATGCAAGAGGGCAAGTGCCTGTA
CAGCCTGGAAGCCATTCCTCTGGAAGATCTGCTGAACAATCCCTTCA
ACTATGAGGTGGACCACATCATCCCCAGAAGCGTGTCCTTCGACAAC
AGCTTCAACAACAAGGTGCTCGTGAAGCAAGAGGAAAACAGCAAGAA
GGGCAACAGAACCCCATTCCAGTACCTGAGCAGCAGCGACAGCAAGA
TCAGCTACGAAACCTTCAAGAAGCACATCCTGAATCTGGCCAAAGGC
AAGGGCAGAATCAGCAAGACCAAGAAAGAATACCTGCTCGAGGAACG
GGACATCAACAGATTCAGCGTGCAGAAAGACTTCATCAATCGGAACC
TGGTGGACACCAGATACGCCACCAGAGGCCTGATGAATCTGCTGCGG
AGCTACTTCAGAGTGAACAATCTGGACGTGAAAGTCAAGTCCATCAA
CGGCGGCTTCACCAGCTTTCTGCGGCGGAAGTGGAAGTTCAAGAAAG
AGCGGAACAAGGGCTATAAGCACCACGCCGAGGACGCCCTGATCATT
GCCAACGCCGATTTCATCTTCAAAGAGTGGAAGAAACTGGACAAGGC
CAAAAAAGTGATGGAAAACCAGATGTTCGAGGAAAAGCAGGCCGAGA
GCATGCCCGAGATCGAAACCGAGCAAGAGTACAAAGAGATTTTCATC
ACGCCCCACCAGATCAAGCACATTAAGGACTTCAAGGACTACAAGTA
CAGCCACCGCGTGGACAAGAAGCCCAATAGAGAGCTGATTAACGACA
CCCTGTACTCTACCCGGAAGGACGACAAGGGCAATACCCTGATCGTC
AACAACCTGAACGGCCTGTACGACAAGGACAACGACAAGCTCAAGAA
GCTGATCAACAAGAGCCCCGAGAAACTGCTGATGTACCACCACGATC
CTCAGACCTACCAGAAACTGAAGCTCATCATGGAACAGTACGGCGAC
GAGAAGAATCCCCTGTACAAGTACTACGAGGAAACCGGGAACTACCT
GACCAAGTACTCCAAAAAGGACAATGGGCCCGTGATCAAGAAGATTA
AGTATTAGGGCAACAAGCTGAATGCCCACCTGGACATCACCGACGAC
TACCCCAACAGCAGAAACAAGGTGGTCAAGCTGTCCCTGAAGCCTTA
CAGATTCGACGTGTACCTGGACAACGGCGTGTACAAGTTCGTGACCG
TGAAGAACCTGGACGTCATCAAAAAAGAAAACTACTAGGAAGTCAAC
AGCAAGTGCTATGAGGAAGCCAAGAAACTCAAGAAAATCAGCAACCA
GGCCGAGTTTATCGCCTCCTTCTACAACAACGATCTGATCAAGATCA
ACGGGGAGCTGTATAGAGTGATCGGCGTGAACAATGACCTGCTGAAC
CGGATCGAAGTGAACATGATCGACATCACCTACCGCGAGTACCTCGA
GAACATGAACGATAAGCGGCCTCCACGGATCATTAAGACAATCGCCA
GCAAGACGCAGAGCATTAAGAAGTACAGCACAGACATCCTGGGCAAC
CTGTACGAAGTGAAGTCTAAGAAGCACCCGCAGATTATCAAGAAAGG
CAGCGGCGCTCCCAAGAAAAAACGGAAGGTTGGCTCCGGAGAGGGCA
GAGGAAGTCTGCTAACATGCGGTGACGTCGAGGAGAATCCTGGCCCA
ATGGAGAGCGACGAGAGCGGCCTGCCCGCCATGGAGATCGAGTGCCG
CATCACCGGCACCCTGAACGGCGTGGAGTTCGAGCTGGTGGGCGGCG
GAGAGGGCACCCCCGAGCAGGGCCGCATGACCAACAAGATGAAGAGC
ACCAAAGGCGCCCTGACCTTCAGCCCCTACCTGCTGAGCCACGTGAT
GGGCTACGGCTTCTACCACTTCGGCACCTACCCCAGCGGCTACGAGA
ACCCCTTCCTGCACGCCATCAACAACGGCGGCTACACCAACACCCGC
ATCGAGAAGTACGAGGACGGCGGCGTGCTGCACGTGAGCTTCAGCTA
CCGCTACGAGGCCGGCCGCGTGATCGGCGACTTCAAGGTGATGGGCA
CCGGCTTCCCCGAGGACAGCGTGATCTTCACCGACAAGATCATCCGC
AGCAACGCCACCGTGGAGCACCTGCACCCCATGGGCGATAACGATCT
GGATGGCAGCTTCACCCGCACCTTCAGCCTGCGCGACGGCGGCTACT
ACAGCTCCGTGGTGGACAGCCACATGCACTTCAAGAGCGCCATCCAC
CCCAGCATCCTGCAGAACGGGGGCCCCATGTTCGCCTTCCGCCGCGT
GGAGGAGGATCACAGCAACACCGAGCTGGGCATCGTGGAGTACCAGC
ACGCCTTCAAGACCCCGGATGCAGATGCCGGTGAAGAATAAGAGCTC
GCTGATCAGCCTCGATAAGATACATTGATGAGTTTGGACAAACCACA
ACTAGAATGCAGTGAAAAAAATGCTTTATTTGTGAAATTTGTGATGC
TATTGCTTTATTTGTAACCATTATAAGCTGCAATAAACAAGTTTTAA
TTAAGGTCGGGCAGGAAGAGGGCCTATTTCCCATGATTCCTTCATAT
TTGCATATACGATACAAGGCTGTTAGAGAGATAATTAGAATTAATTT
GACTGTAAACACAAAGATATTAGTACAAAATACGTGACGTAGAAAGT
AATAATTTCTTGGGTAGTTTGCAGTTTTAAAATTATGTTTTAAAATG
GACTATCATATGCTTACCGTAACTTGAAAGTATTTCGATTTCTTGGC
TTTATATATCTTGTGGAAAGGACGAAACACCGTGCTGCCGCTACCGA
GGATGGGTTTAAGTACTCTGTGCTGGAAACAGCACAGAATCTAGTTA
AACAAGGCAAAATGCCGTGTTTATCTCGTCAACTTGTTGGCGAGATT
TTTTCTAAAGCTTGAATTCAGCTGACGTGCCTCGGACCGCTAGGAAC
CCCTAGTGATGGAGTTGGCCACTCCCTCTCTGCGCGCTCGCTCGCTC
ACTGAGGCCGGGCGACCAAAGGTCGCCCGACGCCCGGGCTTTGCCCG
GGCGGCCTCAGTGAGCGAGCGAGCGCGCAG

Exemplary eGFP with sgRNA plasmids

Exemplary pITR.CMV.eGFP.U6-hsammu386Fw
sequence
(SEQ ID NO: 289)
CTGCGCGCTCGCTCGCTCACTGAGGCCGCCCGGGCGTCGGGCGACCT
TTGGTCGCCCGGCCTCAGTGAGCGAGCGAGCGCGCAGAGAGGGAGTG
GCCAACTCCATCACTAGGGGTTCCTGCGGCCGCACGCGTCTAGATCC
CATATATGGAGTTCCGCGTTACATAACTTACGGTAAATGGCCCGCCT
GGCTGACCGCCCAACGACCCCCGCCCATTGACGTCAATAATGACGTA
TGTTCCCATAGTAACGCCAATAGGGACTTTCCATTGACGTCAATGGG
TGGAGTATTTACGGTAAACTGCCCACTTGGCAGTACATCAAGTGTAT
CATATGCCAAGTACGCCCCCTATTGACGTCAATGACGGTAAATGGCC
CGCCTGGCATTATGCCCAGTACATGACCTTATGGGACTTTCCTACTT
GGCAGTACATCTACGTATTAGTCATCGCTATTACCATGGTGATGCGG
TTTTGGCAGTACATCAATGGGCGTGGATAGCGGTTTGACTCACGGGG
ATTTCCAAGTCTCCACCCCATTGACGTCAATGGGAGTTTGTTTTGGC
ACCAAAATCAACGGGACTTTCCAAAATGTCGTAACAACTCCGCCCCA
TTGACGCAAATGGGCGGTAGGCGTGTACGGTGGGAGGTCTATATAAG
CAGAGCTCGTTTAGTGAACCGTCGTTTAGTGAACCGTcagatcgcct
ggagacgcACCGGTGCCACCATGGTGAGCAAGGGCGAGGAGCTGTTC
ACCGGGGTGGTGCCCATCCTGGTCGAGCTGGACGGCGACGTAAACGG
CCACAAGTTCAGCGTGTCCGGCGAGGGCGAGGGCGATGCCACCTACG
GCAAGCTGACCCTGAAGTTCATCTGCACCACCGGCAAGCTGCCCGTG
CCCTGGCCCACCCTCGTGACCACCCTGACCTACGGCGTGCAGTGCTT
CAGCCGCTACCCCGACCACATGAAGCAGCACGACTTCTTCAAGTCCG
CCATGCCCGAAGGCTACGTCCAGGAGCGCACCATCTTCTTCAAGGAC
GACGGCAACTACAAGACCCGCGCCGAGGTGAAGTTCGAGGGCGACAC
CCTGGTGAACCGCATCGAGCTGAAGGGCATCGACTTCAAGGAGGACG
GCAACATCCTGGGGCACAAGCTGGAGTACAACTACAACAGCCACAAC
GTCTATATCATGGCCGACAAGCAGAAGAACGGCATCAAGGTGAACTT
CAAGATCCGCCACAACATCGAGGACGGCAGCGTGCAGCTCGCCGACC
ACTACCAGCAGAACACCCCCATCGGCGACGGCCCCGTGCTGCTGCCC
GACAACCACTACCTGAGCACCCAGTCCGCCCTGAGCAAAGACCCCAA
CGAGAAGCGCGATCACATGGTCCTGCTGGAGTTCGTGACCGCCGCCG
GGATCACTCTCGGCATGGACGAGCTGTACAAGTAATAATAAGAGCTC
GCTGATCAGCCTCGACTGTGCCTTCTAGTTGCCAGCCATCTGTTGTT
TGCCCCTCCCCCGTGCCTTCCTTGACCCTGGAAGGTGCCACTCCCAC
TGTCCTTTCCTAATAAAATGAGGAAATTGCATCGCATTGTCTGAGTA
GGTGTCATTCTATTCTGGGGGGTGGGGTGGGGCAGGACAGCAAGGGG
GAGGATTGGGAAGACAATAGCAGGCATGCTGGGGATGCGGTGGGCTC
TATGGTTAATTAAGGTCGGGCAGGAAGAGGGCCTATTTCCCATGATT
CCTTCATATTTGCATATACGATACAAGGCTGTTAGAGAGATAATTAG
AATTAATTTGACTGTAAACACAAAGATATTAGTACAAAATACGTGAC
GTAGAAAGTAATAATTTCTTGGGTAGTTTGCAGTTTTAAAATTATGT
TTTAAAATGGACTATCATATGCTTACCGTAACTTGAAAGTATTTCGA
TTTCTTGGCTTTATATATCTTGTGGAAAGGACGAAACACCGAGGAGC
ACCAGGAACTTGCCAGTTTAAGTACTCTGTGCTGGAAACAGCACAGA
ATCTACTTAAACAAGGCAAAATGCCGTGTTTATCTCGTCAACTTGTT
GGCGAGATTTTTTCTAAAGCTTGAATTCAAGCTTGAATTCAGCTGAC
GTGCCTCGGACCGCTAGGAACCCCTAGTGATGGAGTTGGCCACTCCC
TCTCTGCGCGCTCGCTCGCTCACTGAGGCCGGGCGACCAAAGGTCGC
CCGACGCCCGGGCTTTGCCCGGGCGGCCTCAGTGAGCGAGCGAGCGC
GCAG
Exemplary pITR.CMV.eGFP.U6-hsa408Rev
sequence
(SEQ ID NO: 290)
CTGCGCGCTCGCTCGCTCACTGAGGCCGCCCGGGCGTCGGGCGACCT
TTGGTCGCCCGGCCTCAGTGAGCGAGCGAGCGCGCAGAGAGGGAGTG
GCCAACTCCATCACTAGGGGTTCCTGCGGCCGCACGCGTCTAGATCC
CATATATGGAGTTCCGCGTTACATAACTTACGGTAAATGGCCCGCCT
GGCTGACCGCCCAACGACCCCCGCCCATTGACGTCAATAATGACGTA
TGTTCCCATAGTAACGCCAATAGGGACTTTCCATTGACGTCAATGGG
TGGAGTATTTACGGTAAACTGCCCACTTGGCAGTACATCAAGTGTAT
CATATGCCAAGTACGCCCCCTATTGACGTCAATGACGGTAAATGGCC
CGCCTGGCATTATGCCCAGTACATGACCTTATGGGACTTTCCTACTT
GGCAGTACATCTACGTATTAGTCATCGCTATTACCATGGTGATGCGG
TTTTGGCAGTACATCAATGGGCGTGGATAGCGGTTTGACTCACGGGG
ATTTCCAAGTCTCCACCCCATTGACGTCAATGGGAGTTTGTTTTGGC
ACCAAAATCAACGGGACTTTCCAAAATGTCGTAACAACTCCGCCCCA
TTGACGCAAATGGGCGGTAGGCGTGTACGGTGGGAGGTCTATATAAG
CAGAGCTCGTTTAGTGAACCGTCGTTTAGTGAACCGTcagatcgcct
ggagacgcACCGGTGCCACCATGGTGAGCAAGGGCGAGGAGCTGTTC
ACCGGGGTGGTGCCCATCCTGGTCGAGCTGGACGGCGACGTAAACGG
CCACAAGTTCAGCGTGTCCGGCGAGGGCGAGGGCGATGCCACCTACG
GCAAGCTGACCCTGAAGTTCATCTGCACCACCGGCAAGCTGCCCGTG
CCCTGGCCCACCCTCGTGACCACCCTGACCTACGGCGTGCAGTGCTT
CAGCCGCTACCCCGACCACATGAAGCAGCACGACTTCTTCAAGTCCG
CCATGCCCGAAGGCTACGTCCAGGAGCGCACCATCTTCTTCAAGGAC
GACGGCAACTACAAGACCCGCGCCGAGGTGAAGTTCGAGGGCGACAC
CCTGGTGAACCGCATCGAGCTGAAGGGCATCGACTTCAAGGAGGACG
GCAACATCCTGGGGCACAAGCTGGAGTACAACTACAACAGCCACAAC
GTCTATATCATGGCCGACAAGCAGAAGAACGGCATCAAGGTGAACTT
CAAGATCCGCCACAACATCGAGGACGGCAGCGTGCAGCTCGCCGACC
ACTACCAGCAGAACACCCCCATCGGCGACGGCCCCGTGCTGCTGCCC
GACAACCACTACCTGAGCACCCAGTCCGCCCTGAGCAAAGACCCCAA
CGAGAAGCGCGATCACATGGTCCTGCTGGAGTTCGTGACCGCCGCCG
GGATCACTCTCGGCATGGACGAGCTGTACAAGTAATAATAAGAGCTC
GCTGATCAGCCTCGACTGTGCCTTCTAGTTGCCAGCCATCTGTTGTT
TGCCCCTCCCCCGTGCCTTCCTTGACCCTGGAAGGTGCCACTCCCAC
TGTCCTTTCCTAATAAAATGAGGAAATTGCATCGCATTGTCTGAGTA
GGTGTCATTCTATTCTGGGGGGTGGGGTGGGGCAGGACAGCAAGGGG
GAGGATTGGGAAGACAATAGCAGGCATGCTGGGGATGCGGTGGGCTC
TATGGTTAATTAAGGTCGGGCAGGAAGAGGGCCTATTTCCCATGATT
CCTTCATATTTGCATATACGATACAAGGCTGTTAGAGAGATAATTAG
AATTAATTTGACTGTAAACACAAAGATATTAGTACAAAATACGTGAC
GTAGAAAGTAATAATTTCTTGGGTAGTTTGCAGTTTTAAAATTATGT
TTTAAAATGGACTATCATATGCTTACCGTAACTTGAAAGTATTTCGA
TTTCTTGGCTTTATATATCTTGTGGAAAGGACGAAACACCGAAGCCG
AAAACCACGATCATCGTTTAAGTACTCTGTGCTGGAAACAGCACAGA
ATCTACTTAAACAAGGCAAAATGCCGTGTTTATCTCGTCAACTTGTT
GGCGAGATTTTTTCTAAAGCTTGAATTCAAGCTTGAATTCAGCTGAC
GTGCCTCGGACCGCTAGGAACCCCTAGTGATGGAGTTGGCCACTCCC
TCTCTGCGCGCTCGCTCGCTCACTGAGGCCGGGCGACCAAAGGTCGC
CCGACGCCCGGGCTTTGCCCGGGCGGCCTCAGTGAGCGAGCGAGCGC
GCAG
Exemplary pITR.CMV.eGFP.U6-mmu408Rev
sequence
(SEQ ID NO: 291)
CTGCGCGCTCGCTCGCTCACTGAGGCCGCCCGGGCGTCGGGCGACCT
TTGGTCGCCCGGCCTCAGTGAGCGAGCGAGCGCGCAGAGAGGGAGTG
GCCAACTCCATCACTAGGGGTTCCTGCGGCCGCACGCGTCTAGATCC
CATATATGGAGTTCCGCGTTACATAACTTACGGTAAATGGCCCGCCT
GGCTGACCGCCCAACGACCCCCGCCCATTGACGTCAATAATGACGTA
TGTTCCCATAGTAACGCCAATAGGGACTTTCCATTGACGTCAATGGG
TGGAGTATTTACGGTAAACTGCCCACTTGGCAGTACATCAAGTGTAT
CATATGCCAAGTACGCCCCCTATTGACGTCAATGACGGTAAATGGCC
CGCCTGGCATTATGCCCAGTACATGACCTTATGGGACTTTCCTACTT
GGCAGTACATCTACGTATTAGTCATCGCTATTACCATGGTGATGCGG
TTTTGGCAGTACATCAATGGGCGTGGATAGCGGTTTGACTCACGGGG
ATTTCCAAGTCTCCACCCCATTGACGTCAATGGGAGTTTGTTTTGGC
ACCAAAATCAACGGGACTTTCCAAAATGTCGTAACAACTCCGCCCCA
TTGACGCAAATGGGCGGTAGGCGTGTACGGTGGGAGGTCTATATAAG
CAGAGCTCGTTTAGTGAACCGTCGTTTAGTGAACCGTcagatcgcct
ggagacgcACCGGTGCCACCATGGTGAGCAAGGGCGAGGAGCTGTTC
ACCGGGGTGGTGCCCATCCTGGTCGAGCTGGACGGCGACGTAAACGG
CCACAAGTTCAGCGTGTCCGGCGAGGGCGAGGGCGATGCCACCTACG
GCAAGCTGACCCTGAAGTTCATCTGCACCACCGGCAAGCTGCCCGTG
CCCTGGCCCACCCTCGTGACCACCCTGACCTACGGCGTGCAGTGCTT
CAGCCGCTACCCCGACCACATGAAGCAGCACGACTTCTTCAAGTCCG
CCATGCCCGAAGGCTACGTCCAGGAGCGCACCATCTTCTTCAAGGAC
GACGGCAACTACAAGACCCGCGCCGAGGTGAAGTTCGAGGGCGACAC
CCTGGTGAACCGCATCGAGCTGAAGGGCATCGACTTCAAGGAGGACG
GCAACATCCTGGGGCACAAGCTGGAGTACAACTACAACAGCCACAAC
GTCTATATCATGGCCGACAAGCAGAAGAACGGCATCAAGGTGAACTT
CAAGATCCGCCACAACATCGAGGACGGCAGCGTGCAGCTCGCCGACC
ACTACCAGCAGAACACCCCCATCGGCGACGGCCCCGTGCTGCTGCCC
GACAACCACTACCTGAGCACCCAGTCCGCCCTGAGCAAAGACCCCAA
CGAGAAGCGCGATCACATGGTCCTGCTGGAGTTCGTGACCGCCGCCG
GGATCACTCTCGGCATGGACGAGCTGTACAAGTAATAATAAGAGCTC
GCTGATCAGCCTCGACTGTGCCTTCTAGTTGCCAGCCATCTGTTGTT
TGCCCCTCCCCCGTGCCTTCCTTGACCCTGGAAGGTGCCACTCCCAC
TGTCCTTTCCTAATAAAATGAGGAAATTGCATCGCATTGTCTGAGTA
GGTGTCATTCTATTCTGGGGGGTGGGGTGGGGCAGGACAGCAAGGGG
GAGGATTGGGAAGACAATAGCAGGCATGCTGGGGATGCGGTGGGCTC
TATGGTTAATTAAGGTCGGGCAGGAAGAGGGCCTATTTCCCATGATT
CCTTCATATTTGCATATACGATACAAGGCTGTTAGAGAGATAATTAG
AATTAATTTGACTGTAAACACAAAGATATTAGTACAAAATACGTGAC
GTAGAAAGTAATAATTTCTTGGGTAGTTTGCAGTTTTAAAATTATGT
TTTAAAATGGACTATCATATGCTTACCGTAACTTGAAAGTATTTCGA
TTTCTTGGCTTTATATATCTTGTGGAAAGGACGAAACACCGAAGCCA
AAGACCACAATCATCGTTTAAGTACTCTGTGCTGGAAACAGCACAGA
ATCTACTTAAACAAGGCAAAATGCCGTGTTTATCTCGTCAACTTGTT
GGCGAGATTTTTTCTAAAGCTTGAATTCAAGCTTGAATTCAGCTGAC
GTGCCTCGGACCGCTAGGAACCCCTAGTGATGGAGTTGGCCACTCCC
TCTCTGCGCGCTCGCTCGCTCACTGAGGCCGGGCGACCAAAGGTCGC
CCGACGCCCGGGCTTTGCCCGGGCGGCCTCAGTGAGCGAGCGAGCGC
GCAG

xxi. AAV Particles

Among other things, the present disclosure provides AAV particles that comprise a construct encoding a KCNQ4 gene or characteristic portion thereof and/or an inhibitory nucleic acid sequence as described herein, and a capsid described herein. In some embodiments, AAV particles can be described as having a serotype, which is a description of a construct strain (e.g., serotype of viral components, e.g., ITRs), and a capsid strain. For example, in some embodiments an AAV particle may be described as AAV2, wherein the particle has an AAV2 capsid and a construct that comprises characteristic AAV2 Inverted Terminal Repeats (ITRs). In some embodiments, an AAV particle may be described as a pseudotype, wherein a capsid and construct are derived from different AAV strains, for example, AAV2/9 would refer to an AAV particle that comprises a construct utilizing AAV2 ITRs and an AAV9 capsid. In some embodiments, an AAV particle is described as Anc80, wherein a particle has an Anc80 capsid and AAV2 ITRs. In some embodiments, a construct may be described, e.g., as provided herein, without specific mention of a serotype of, e.g., an ITR in the name of the construct; however, it will be evident to one of skill in the art, reading the disclosure, what type of ITR is present in a given construct, e.g., AAV2 ITR. In some embodiments, an AAV particle of the present disclosure comprises at least one construct, which construct can be or comprise any sequence disclosed herein.

e. KCNQ4 Genome Editing

In some embodiments, a genome editing system targets nucleotides within a specific target site. In some such embodiments, a target site is or comprises a loss-of-function KCNQ4 variant sequence.

In some embodiments, a genome editing system comprises a nucleic acid strand that is complementary to a target site in a KCNQ4 gene product (e.g., complementary to a nucleotide sequence that is at least 85%, 90%, 91%, 92%, 93%, 94%, 95%, 96%, 97%, 98%, 99%, or 100% identical to a portion of any of SEQ ID NOs: 1-10 and/or 25-30 and/or 90-91) or a characteristic portion thereof. In some embodiments, a target site may be 15-30 nucleotides long, e.g., 15, 16, 17, 18, 19, 20, 21, 22, 23, 24, 25, 26, 27, 28, 29, or 30 nucleotides long, although shorter and longer target sites are also contemplated.

In some embodiments, a genome editing system comprises a nucleic acid strand that comprises a region that is perfectly complementary to at least 6, 7, 8, 9, 10, 11, 12, 13 14, 15, 16, 17, 18, 19, 20, 21, 22, 23, 24, 25, 26, 27, 28, 29, or 30 consecutive nucleotides of a KCNQ4 gene product. In some embodiments, a KCNQ gene product is or comprises any of SEQ ID NOs: 1-10 and/or 25-30 and/or 90-91 or a characteristic portion thereof). In some embodiments such an RNA-guided nuclease system is capable of inhibiting expression of KCNQ4 of one or more non-human species, e.g., a non-human primate KCNQ4, e.g., Macaca fascicularis KCNQ4, in addition to human KCNQ4. The Macaca fascicularis KCNQ4 gene has been assigned NCBI Gene ID: 102143586 and predicted amino acid and nucleotide sequences of Macaca fascicularis KCNQ4 are listed under NCBI RefSeq accession numbers XP_005543852 and XM_005543795.2, respectively.

In some embodiments, a genome editing system is complementary to a target site that is identical in human and Macaca fascicularis KCNQ4 transcripts. In some embodiments, a genome editing system is complementary to a target site of a human KCNQ4 transcript that differs by 1, 2, or 3 nucleotides from a sequence in a Macaca fascicularis KCNQ4 transcript. It will be appreciated that a genome editing system that targets human KCNQ4 may also target non-primate KCNQ4, e.g., rat or mouse KCNQ4, particularly if conserved regions of KCNQ4 transcript are targeted.

i. RNA-Guided Nucleases

RNA-guided nucleases according to the present disclosure include, but are not limited to, naturally-occurring Class 2 CRISPR nucleases such as Cas9, and Cpf1, as well as other nucleases derived or obtained therefrom. In functional terms, RNA-guided nucleases are defined as those nucleases that: (a) interact with (e.g., complex with) a gRNA; and (b) together with gRNA, associate with, and optionally cleave or modify, a target region of a DNA that includes (i) a sequence complementary to a targeting domain of a gRNA and, optionally, (ii) an additional sequence referred to as a “protospacer adjacent motif,” or “PAM,” which is described in greater detail herein.

Naturally occurring CRISPR systems are organized evolutionarily into two classes and five types (Makarova et al. Nat Rev Microbiol. 2011 June; 9(6): 467-477 (“Makarova”), which is incorporated in its entirety herein by reference), and while genome editing systems of the present disclosure may adapt components of any type or class of naturally occurring CRISPR system, embodiments presented herein are generally adapted from Class 2, and type II or V CRISPR systems. Class 2 systems, which encompass types II and V, are characterized by relatively large, multidomain CRISPR proteins (e.g., Cas9 or Cpf1) and one or more gRNAs (e.g., a crRNA and, optionally, a tracrRNA) that form ribonucleoprotein (RNP) complexes that associate with (i.e., target) and cleave specific loci complementary to a targeting (or spacer) sequence of a crRNA. Genome editing systems according to the present disclosure similarly target and edit cellular DNA sequences, but differ significantly from CRISPR systems occurring in nature. For example, unimolecular gRNAs described herein do not occur in nature, and both gRNAs and CRISPR nucleases according to this disclosure may incorporate any number of non-naturally occurring modifications.

As described herein, it should be noted that a genome editing systems of the present disclosure can be targeted to a single specific nucleotide sequence, or may be targeted to—and capable of editing in parallel—two or more specific nucleotide sequences through use of two or more gRNAs. In some embodiments, use of multiple gRNAs is referred to as “multiplexing.” As described herein, multiplexing can be employed, for example, to target multiple, unrelated target sequences of interest, or to form multiple SSBs or DSBs within a single target domain and, in some cases, to generate specific edits within such target domain. For example, International Patent Publication No. WO 2015/138510 by Maeder et al., which is incorporated in its entirety herein by reference; (“Maeder”) describes a genome editing system for correcting a point mutation (C.2991+1655A to G) in human CEP290 that results in t creation of a cryptic splice site, which in turn reduces or eliminates function of the gene. That genome editing system of Maeder utilizes two gRNAs targeted to sequences on either side of (i.e., flanking) the point mutation, and forms DSBs that flank the mutation. This, in turn, promotes deletion of the intervening sequence, including the mutation, thereby eliminating the cryptic splice site and restoring normal gene function.

As another example, WO 2016/073990 by Cotta-Ramusino, et al. (“Cotta-Ramusino”), which is incorporated in its entirety herein by reference. Cotta-Ramusino describes a genome editing system that utilizes two gRNAs in combination with a Cas9 nickase (a Cas9 that makes a single strand nick such as S. pyogenes D10A), an arrangement termed a “dual-nickase system.” The dual-nickase system of Cotta-Ramusino is configured to make two nicks on opposite strands of a sequence of interest that are offset by one or more nucleotides, which nicks combine to create a double strand break having an overhang (5′ in the case of Cotta-Ramusino, though 3′ overhangs are also possible). The overhang, in turn, can facilitate homology directed repair events in some circumstances. And, as another example, WO 2015/070083 by Palestrant et al., which is incorporated in its entirety herein by reference; (“Palestrant”) describes a gRNA targeted to a nucleotide sequence encoding Cas9 (referred to as a “governing RNA”), which can be included in a genome editing system comprising one or more additional gRNAs to permit transient expression of a Cas9 that might otherwise be constitutively expressed, for example in some virally transduced cells. These multiplexing applications are intended to be exemplary, rather than limiting, and the skilled artisan will appreciate that other applications of multiplexing are generally compatible with the genome editing systems described here.

Genome editing systems can, in some instances, form double strand breaks that are repaired by cellular DNA double-strand break mechanisms such as NHEJ or HDR. These mechanisms are described throughout the literature, for example by Davis & Maizels, PNAS, 111(10):E924-932, Mar. 11, 2014, which is incorporated in its entirety herein by reference (“Davis”) (describing Alt-HDR); Frit et al. DNA Repair 17(2014) 81-97, which is incorporated in its entirety herein by reference (“Frit”) (describing Alt-NHEJ); and Iyama and Wilson III, DNA Repair (Amst.) 2013-August; 12(8): 620-636, which is incorporated in its entirety herein by reference (“Iyama”) (describing canonical HDR and NHEJ pathways generally).

Where genome editing systems operate by forming DSBs, such systems optionally include one or more components that promote or facilitate a particular mode of double-strand break repair or a particular repair outcome. For instance, Cotta-Ramusino also describes genome editing systems in which a single stranded oligonucleotide “donor template” is added; a donor template is incorporated into a target region of cellular DNA that is cleaved by a genome editing system, and can result in a change in a target sequence.

In some embodiments, genome editing systems modify a target sequence, or modify expression of a gene in or near a target sequence, without causing single- or double-strand breaks. For example, a genome editing system may include a CRISPR protein fused to a functional domain that acts on DNA, thereby modifying a target sequence or its expression. As one example, a CRISPR protein can be connected to (e.g., fused to) a cytidine deaminase functional domain, and may operate by generating targeted C-to-A substitutions. Exemplary nuclease/deaminase fusions are described in Komor et al. Nature 533, 420-424 (19 May 2016) (“Komor”), which is incorporated in its entirety herein by reference. In some embodiments, a genome editing system may utilize a cleavage-inactivated (i.e., a “dead”) nuclease, such as a dead Cas9 (dCas9), and may operate by forming stable complexes on one or more targeted regions of cellular DNA, thereby interfering with functions involving a targeted region(s) including, without limitation, mRNA transcription, chromatin remodeling, etc. In some embodiments, a genome editing system may be self-inactivating to improve a safety profile, as described by Li et al. “A Self-Deleting AAV-CRISPR System for In vivo Editing” Mol Ther Methods Clin Dev. 2019 Mar. 15; 12: 111-122; published online (2018 Dec. 6), the contents of which are hereby incorporated by reference in its entirety.

As the following examples will illustrate, RNA-guided nucleases can be defined, in broad terms, by their PAM specificity and cleavage activity, even though variations may exist between individual RNA-guided nucleases that share the same PAM specificity or cleavage activity. Skilled artisans will appreciate that some aspects of the present disclosure relate to systems, methods and compositions that can be implemented using any suitable RNA-guided nuclease having a certain PAM specificity and/or cleavage activity. For this reason, unless otherwise specified, the term RNA-guided nuclease should be understood as a generic term, and not limited to any particular type (e.g., Cas9 vs. Cpf1), species (e.g., S. pyogenes vs. S. aureus, etc.) or variation (e.g., full-length vs. truncated or split; naturally-occurring PAM specificity vs. engineered PAM specificity, etc.) of RNA-guided nuclease. In some embodiments, a CRISPR/Cas is derived from a type II CRISPR/Cas system. In some embodiments, a CRISPR/Cas system is derived from a Cas9 protein. A Cas9 protein can be from Streptococcus pyogenes, Streptococcus thermophilus, Staphylococcus aureus, Campylobacter jejuni, or other species. In some embodiments, Cas9 can include: spCas9, Cpf1, CasY, CasX, saCas9, or CjCas9.

Administering bacterial Cas9 in humans presents immunogenicity concerns. Therefore, it is important to develop a codon-optimized CRISPR system as described herein to reduce immunogenicity. In addition, some other limitations include a need to use a two construct system (instead of a single construct system such that is used in shRNA and miRNA protocols), and determination of off-target risk (e.g., even if using dCas9 to reduce expression of KCNQ4, there may be repression of other targets besides from KCNQ4)

A PAM sequence takes its name from its sequential relationship to a “protospacer” sequence that is complementary to gRNA targeting domains (or “spacers”). Together with protospacer sequences, PAM sequences define target regions or sequences for specific RNA-guided nuclease/gRNA combinations.

Various RNA-guided nucleases may require different sequential relationships between PAMs and protospacers. In general, Cas9s recognize PAM sequences that are 3′ of a protospacer. Cpf1, on the other hand, generally recognizes PAM sequences that are 5′ of a protospacer.

In addition to recognizing specific sequential orientations of PAMs and protospacers, RNA-guided nucleases can also recognize specific PAM sequences. S. aureus Cas9, for instance, recognizes a PAM sequence of NNGRRT or NNGRRV, wherein the N residues are immediately 3′ of the region recognized by the gRNA targeting domain. S. pyogenes Cas9 recognizes NGG PAM sequences. And F. novicida Cpf1 recognizes a TTN PAM sequence. PAM sequences have been identified for a variety of RNA-guided nucleases, and a strategy for identifying novel PAM sequences has been described by Shmakov et al., 2015, Molecular Cell 60, 385-397, Nov. 5, 2015. It should also be noted that engineered RNA-guided nucleases can have PAM specificities that differ from\ PAM specificities of reference molecules (for instance, in the case of an engineered RNA-guided nuclease, a reference molecule may be a naturally occurring variant from which an RNA-guided nuclease is derived, or a naturally occurring variant having the greatest amino acid sequence homology to an engineered RNA-guided nuclease).

In addition to their PAM specificity, RNA-guided nucleases can be characterized by their DNA cleavage activity: naturally-occurring RNA-guided nucleases typically form DSBs in target nucleic acids, but engineered variants have been produced that generate only SSBs (discussed above) Ran & Hsu, et al., Cell 154(6), 1380-1389, Sep. 12, 2013 (“Ran”)), or that that do not cut at all.

CRISPR Fusion Proteins

As described herein, in some embodiments, a CRISPR nuclease is part of a fusion protein comprising one or more heterologous protein domains (e.g., about or more than about 1, 2, 3, 4, 5, 6, 7, 8, 9, 10, or more domains in addition to a CRISPR nuclease). A CRISPR nuclease fusion protein may comprise any additional protein sequence, and optionally a linker sequence between any two domains. Examples of protein domains that may be fused to a CRISPR nuclease include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Additional domains that may form part of a fusion protein comprising a CRISPR nuclease are described in US20110059502, incorporated herein by reference. In some embodiments, a tagged CRISPR nuclease is used to identify a location of a target sequence. In some embodiments, a CRISPR nuclease that is part of a fusion protein has been engineered to produce only SSBs as described herein. In some embodiments, a CRISPR nuclease that is part of a fusion protein has been engineered to not cut at all as described herein.

CRISPR Variants

In general, RNA-guided nucleases comprise at least one RNA recognition and/or RNA binding domain. RNA recognition and/or RNA binding domains interact with a guiding RNA. CRISPR/Cas proteins can also comprise nuclease domains (i.e., DNase or RNase domains), DNA binding domains, helicase domains, RNAse domains, protein-protein interaction domains, dimerization domains, as well as other domains. RNA-guided nucleases can be modified to increase nucleic acid binding affinity and/or specificity, alter an enzymatic activity, and/or change another property of a protein. In some embodiments, a CRISPR/Cas-like protein of a fusion protein can be derived from a wild type Cas9 protein or fragment thereof. In other embodiments, a CRISPR/Cas can be derived from modified Cas9 protein. For example, an amino acid sequence of a Cas9 protein can be modified to alter one or more properties (e.g., nuclease activity, affinity, stability, and so forth) of a protein. Alternatively, domains of a Cas9 protein not involved in RNA-guided cleavage can be eliminated from a protein such that a modified Cas9 protein is smaller than a wild type Cas9 protein. In general, a Cas9 protein comprises at least two nuclease (i.e., DNase) domains. For example, a Cas9 protein can comprise a RuvC-like nuclease domain and a HNH-like nuclease domain. RuvC and HNH domains work together to cut single strands to make a double-stranded break in DNA (Jinek et al., 2012, Science, 337:816-821, which is incorporated in its entirety herein by reference).

In some embodiments, a Cas9-derived protein can be modified to contain only one functional nuclease domain (either a RuvC-like or a HNH-like nuclease domain). For example, a Cas9-derived protein can be modified such that one nuclease domain is deleted or mutated such that it is no longer functional (i.e., nuclease activity is absent). In some embodiments in which one nuclease domains is inactive, a Cas9-derived protein is able to introduce a nick into a double-stranded nucleic acid (such protein is termed a “nickase”), but not cleave double-stranded DNA. In any of the above-described embodiments, any or all of nuclease domains can be inactivated by one or more deletion mutations, insertion mutations, and/or substitution mutations using well-known methods, such as site-directed mutagenesis, PCR-mediated mutagenesis, and total gene synthesis, as well as other methods known in the art.

One example of a CRISPR/Cas9 system used to inhibit gene expression, CRISPRi, is described in U.S. Publication No. US2014/0068797, which is incorporated herein by reference in its entirety. CRISPRi induces permanent gene disruption that utilizes the RNA-guided Cas9 endonuclease to introduce DNA double stranded breaks which trigger error-prone repair pathways to result in frame shift mutations. A catalytically dead Cas9 lacks endonuclease activity. When coexpressed with a gRNA, a DNA recognition complex is generated that specifically interferes with transcriptional elongation, RNA polymerase binding, or transcription factor binding. This CRISPRi system efficiently represses expression of targeted genes.

ii. Guide RNAs (gRNAs)

gRNA Sequence Selection

A gRNA sequence may be specific for any gene, such as a gene that would affect (e.g., ameliorate, improve, attenuate, mitigate) hearing loss. In some embodiments, a gene encodes an ion channel subunit. In some such embodiments, a gene is KCNQ4. In some embodiments, a gRNA sequence includes an RNA sequence, a DNA sequence, a combination thereof (a RNA-DNA combination sequence), or a sequence with synthetic nucleotides. A gRNA sequence can be a single molecule or a double molecule. In one embodiment, a gRNA sequence comprises a single guide RNA (sgRNA).

In some embodiments, a gRNA sequence is specific for a gene and targets that gene for Cas endonuclease-induced double strand breaks. A sequence of a gRNA may be within a loci of the gene. In one embodiment, a gRNA sequence is at least 10, 11, 12, 13, 14, 15, 16, 17, 18, 19, 20, 21, 22, 23, 24, 25, 26, 27, 30, 31, 32, 33, 34, 35, 36, 37, 38, 39, 40 or more nucleotides in length. In some embodiments, a gRNA sequence is from about 18 to about 22 nucleotides in length.

As described herein, in some embodiments in the context of formation of a CRISPR complex, “target sequence” refers to a sequence to which a guide sequence is designed to have some complementarity, where hybridization between a target sequence and a guide sequence promotes formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. In some embodiments, a target sequence is located in the nucleus or cytoplasm of a cell. In other embodiments, a target sequence may be within an organelle of a eukaryotic cell, for example, mitochondrion or nucleus. Typically, in the context of an endogenous CRISPR system, formation of a CRISPR complex (comprising a guide sequence hybridized to a target sequence and complexed with one or more Cas proteins) results in cleavage of one or both strands in or near (e.g., within about 1, 2, 3, 4, 5, 6, 7, 8, 9, 10, 20, 50 or more base pairs) a target sequence. As with a target sequence, it is believed that complete complementarity is not needed, provided this is sufficient to be functional. In some embodiments, a tracr sequence has at least 50%, 60%, 70%, 80%, 90%, 95% or 99% of sequence complementarity along the length of a tracr mate sequence when optimally aligned.

In some embodiments, a gRNA sequence targets a KCNQ4 gene.

TABLE 2

| sequence | | 5′-gRNA Target |
|---|---|---|
| Number | Name | sequence-3′ |
| SEQ ID | guide RNA # 386Fw | AGGAGCACCAG |
| NO: 42 | | GAACTTGCCA |
| SEQ ID | guide RNA # | AAGCCGAAAAC |
| NO: 43 | 408Rev | CACGATCATC |
| SEQ ID | guide RNA # 233Fw | GCGCTACCGCC |
| NO: 44 | | GCCTGCAGAA |
| SEQ ID | guide RNA # 447Fw | TTTCGGCTTGG |
| NO: 45 | | AGTACATCGT |
| SEQ ID | guide RNA # 482Fw | CCGGATGCTGC |
| NO: 46 | | TGCCGCTACC |
| SEQ ID | guide RNA # 490Fw | TGCTGCCGCTA |
| NO: 47 | | CCGAGGATGG |

gRNA Design

Methods for selection and validation of target sequences as well as off-target analyses have been described previously, e.g., in Mali; Hsu; Fu et al., 2014 Nat biotechnol 32(3): 279-84, Heigwer et al., 2014 Nat methods 11(2):122-3; Bae et al. (2014) Bioinformatics 30(10): 1473-5; and Xiao A et al. (2014) Bioinformatics 30(8): 1180-1182, each of which is incorporated in its entirety herein by reference. As a non-limiting example, gRNA design may involve use of a software tool to optimize choice of potential target sequences corresponding to a user's target sequence, e.g., to minimize total off-target activity across a genome. While off-target activity is not limited to cleavage, cleavage efficiency at each off-target sequence can be predicted, e.g., using an experimentally-derived weighting scheme. These and other guide selection methods are described in detail in Maeder and Cotta-Ramusino.

For example, methods for selection and validation of target sequences as well as off-target analyses can be performed using cas-offinder (Bae S, Park J, Kim J-S. Cas-OFFinder: a fast and versatile algorithm that searches for potential off-target sites of Cas9 RNA-guided endonucleases. Bioinformatics. 2014; 30:1473-5, which is incorporated in its entirety herein by reference). Cas-offinder is a tool that can quickly identify all sequences in a genome that have up to a specified number of mismatches to a guide sequence.

As another example, methods for scoring how likely a given sequence is to be an off-target (e.g., once candidate target sequences are identified) can be performed. An exemplary score includes a Cutting Frequency Determination (CFD) score, as described by Doench J G, Fusi N, Sullender M, Hegde M, Vaimberg E W, Donovan K F, et al. Optimized sgRNA design to maximize activity and minimize off-target effects of CRISPR-Cas9. Nat Biotechnol. 2016; 34:184-91, which is incorporated in its entirety herein by reference.

gRNA Modifications

Activity, stability, or other characteristics of gRNAs can be altered through incorporation of certain modifications. As one example, transiently expressed or delivered nucleic acids can be prone to degradation by, e.g., cellular nucleases. Accordingly, gRNAs described herein can contain one or more modified nucleosides or nucleotides that can introduce stability toward nucleases. While not wishing to be bound by theory, it is also believed that certain modified gRNAs described herein can exhibit a reduced innate immune response when introduced into cells. Those of skill in the art will be aware of certain cellular responses commonly observed in cells, e.g., mammalian cells, in response to exogenous nucleic acids, particularly those of viral or bacterial origin. Such responses, which can include induction of cytokine expression and release and cell death, may be reduced or eliminated altogether by modifications presented herein.

Certain exemplary modifications discussed in this section can be included at any position within a gRNA sequence including, without limitation at or near its 5′ end (e.g., within 1-10, 1-5, or 1-2 nucleotides of a 5′ end) and/or at or near its 3′ end (e.g., within 1-10, 1-5, or 1-2 nucleotides of a 3′ end). In some cases, modifications are positioned within functional motifs, such as a repeat-anti-repeat duplex of a Cas9 gRNA, a stem loop structure of a Cas9 or Cpf1 gRNA, and/or a targeting domain of a gRNA. Others types of modified nucleobases are described herein.

f. KCNQ4 Knockdown

The present disclosure provides technologies (e.g., comprising compositions) that may, in some embodiments, reduce, suppress or otherwise decrease (“knock down”) expression of one or more gene products. For example, in some embodiments, technologies of the present disclosure may achieve knockdown of a KCNQ4 gene product (e.g., a KCNQ4 gene, mRNA, protein, etc.). In some embodiments, a KCNQ4 gene product may be a wild-type KCNQ4 or may have one or more mutations relative to a wild-type sequence, e.g., a loss-of-function KCNQ4 variant.

In some embodiments, knockdown of a KCNQ4 gene product (e.g., a KCNQ4 gene, mRNA, protein, etc.) is achieved using one or more techniques to inhibit one or more gene products or processes by which gene products are produced. For example, in some embodiments, the present disclosure provides technologies that comprise compositions that are or comprise inhibitory nucleic acid molecules to knock down expression of a gene product (e.g., a KCNQ4 gene product).

In some embodiments, an inhibitory nucleic acid molecule targets nucleotides within a KCNQ4 gene product.

In some embodiments, an inhibitory nucleic acid molecule comprises a nucleic acid strand that is complementary to a target site of a KCNQ4 gene product, e.g., KCNQ4 mRNA (e.g., complementary to a nucleotide sequence that is at least 85%, 90%, 91%, 92%, 93%, 94%, 95%, 96%, 97%, 98%, 99%, or 100% identical to a portion of SEQ ID NOs: 1-10 and/or 25-30 and/or 90-91). In some embodiments, a target site may be 15-30 nucleotides long, e.g., 15, 16, 17, 18, 19, 20, 21, 22, 23, 24, 25, 26, 27, 28, 29, or 30 nucleotides long, although shorter and longer target sites are also contemplated.

In some embodiments, an inhibitory nucleic acid molecule comprises a nucleic acid strand that comprises a region that is perfectly complementary to at least 6, 7, 8, 9, 10, 11, 12, 13 14, 15, 16, 17, 18, 19, 20, 21, 22, 23, 24, 25, 26, 27, 28, 29, or 30 consecutive nucleotides of any of SEQ ID NOs:1-10 or 25-30 or 90-91 or characteristic portions thereof).

In some embodiments an inhibitory nucleic acid molecule is capable of inhibiting expression of a KCNQ4 gene product of one or more non-human species, e.g., a non-human primate KCNQ4, e.g., Macaca fascicularis KCNQ4, in addition to human KCNQ4. A Macaca fascicularis KCNQ4 gene has been assigned NCBI Gene ID: 102143586 and predicted amino acid and nucleotide sequences of Macaca fascicularis KCNQ4 are listed under NCBI RefSeq accession numbers XP 005543852.2 and XM_005543795.2, respectively. In some embodiments, an inhibitory RNA molecule or Genome editing system is complementary to a target portion that is identical in human and Macaca fascicularis KCNQ4 transcripts. In some embodiments, an inhibitory RNA molecule is complementary to a target site of a human KCNQ4 transcript that differs by 1, 2, or 3 nucleotides from a sequence in a Macaca fascicularis KCNQ4 transcript. It will be appreciated that an inhibitory RNA molecule that inhibits expression of human KCNQ4 gene product may also inhibit expression of non-primate KCNQ4, e.g., rat or mouse KCNQ4, particularly if conserved regions of KCNQ4 transcript are targeted.

i. Inhibitory Nucleic Acid Molecules

RNA interference (RNAi) is a process of sequence-specific post-transcriptional gene silencing by which, e.g., double stranded RNA (dsRNA) homologous to a target locus can specifically inactivate gene function (Hammond et al., Nature Genet. 2001; 2:110-119; Sharp, Genes Dev. 1999; 13:139-141). For example, positional location of shRNAs targeting intronic-3XmiR, polyA-3XmiR, or both intronic-3XmiR and PolyA-3XmiR reduced PIZ serum level (% knockdown as compared to GFP control) (Mueller et al 2012). As described herein, positional impacts of miRNAs are tested and evaluated. In some embodiments, dsRNA-induced gene silencing can be mediated by short double-stranded small interfering RNAs (siRNAs) generated from longer dsRNAs by ribonuclease III cleavage (Bernstein et al., Nature 2001; 409:363-366 and Elbashir et al., Genes Dev. 2001; 15:188-200). Without being bound by any particular theory, RNAi-mediated gene silencing is thought to occur via sequence-specific RNA degradation, where sequence specificity is determined by interaction of a siRNA with its complementary sequence within a target RNA (see, e.g., Tuschl, Chem. Biochem. 2001; 2:239-245). In some embodiments, RNAi can involve use of, e.g., siRNAs (Elbashir, et al., Nature 2001; 411: 494-498, which is incorporated in its entirety herein by reference) or short hairpin RNAs (shRNAs) bearing a fold back stem-loop structure (Paddison et al., Genes Dev. 2002; 16: 948-958; Sui et al., Proc. Natl. Acad. Sci. USA 2002; 99:5515-5520; Brummelkamp et al., Science 2002; 296:550-553; Paul et al., Nature Biotechnol. 2002; 20:505-508, each of which is incorporated in its entirety herein by reference).

In some embodiments, an inhibitory nucleic acid molecule is designed on a patient-by-patient basis in accordance with the present disclosure. For example, in some embodiments, a patient with a history (e.g., parent or symptoms of hearing loss) of hearing loss or at risk of hearing loss may be evaluated (e.g., have samples diagnostically analyzed) for one or more variants (e.g., substitutions, additions, deletions, etc.) in one or more genes. In some such embodiments, identified variants (e.g., mutations) may be novel (i.e., not previously described in the literature), and inhibitory nucleic acid therapeutics will be personalized to variant(s) (e.g., mutation(s)) of a particular patient.

In some embodiments an inhibitory nucleic acid is one or more of a short interfering RNA (siRNA), a short hairpin RNA (shRNA), an antisense oligonucleotide, or a ribozyme. In some embodiments, knockdown of KCNQ4 expression is achieved via inhibitory nucleic acids that target a KCNQ4 sequence as described herein. In some such embodiments, a targeted KCNQ4 sequence may be a wild-type and/or pathogenic KCNQ4 variant gene product.

In some embodiments, an inhibitory nucleic acid of the present disclosure may be used to decrease expression of a KCNQ4 gene product (e.g., a loss-of-function KCNQ4 variant gene product). In some such embodiments, a construct encodes an inhibitory nucleic acid that may, in some embodiments, decrease expression of a KCNQ4 gene product, e.g., in a human cell (e.g., a hair cell, e.g., an outer hair cell). Non-limiting examples of siRNAs that can decrease expression of a KCNQ4 gene product, e.g., in a human cell (e.g., a hair cell, e.g., an outer hair cell) are provided herein In some embodiments, after an inhibitory nucleic acid is used to decrease expression of a loss-of-function KCNQ4 variant gene product, another (i.e., non-inhibitory) nucleic acid molecule may be used to express functional KCNQ4 protein. The present disclosure recognizes that, in some such embodiments, it is contemplated that wild-type or other functional (e.g., codon-optimized) KCNQ4 gene products may be vulnerable to miRNA degradation. Accordingly, in some such embodiments, a KCNQ4 sequence that is used to express a functional KCNQ4 gene product may be a codon-optimized KCNQ4 sequence.

siRNA or shRNA

In some embodiments, the present disclosure provides an inhibitory nucleic acid e, e.g., a chemically-modified siRNAs or a construct-driven expression of short hairpin RNA (shRNA) that are then cleaved to siRNA, e.g., within a cell. Accordingly, one of skill in the art will understand that, for purposes of sequences, an shRNA sequence is interchangeable with an siRNA sequence and that where the disclosure refers to an siRNA, an shRNA sequence may be used since the shRNA will be cleaved into siRNA. For example, in some embodiments, an inhibitory nucleic acid can be a dsRNA (e.g., siRNA) including 16-30 nucleotides, e.g., 16, 17, 18, 19, 20, 21, 22, 23, 24, 25, 26, 27, 28, 29, or 30 nucleotides in each strand, where one strand is substantially identical, e.g., at least 80% (or more, e.g., 85%, 90%, 95%, or 100%) identical, e.g., having 3, 2, 1, or 0 mismatched nucleotide(s), to a target region in a potassium-channel (e.g., Kv7.4) encoding mRNA, and the other strand is complementary to the first strand. In some embodiments, dsRNA molecules can be designed using methods known in the art, e.g., Dharmacon.com (see, siDESIGN CENTER) or “The siRNA User Guide,” available on the Internet at mpibpc.gwdg.de/abteilungen/100/105/sirna.html website which is incorporated in its entirety herein by reference. Without being bound by any particular theory, the present disclosure contemplates that siRNA or shRNAs are more “endogenous” (e.g., no foreign proteins) in a way that may be more recognizable to a cell compared to other available techniques that will be known to those of skill in the art. Accordingly, in some embodiments, siRNA or shRNA have lower immunogenicity and/or have less risk of off-target DNA cleavage as compared to other techniques known to those of skill in the art.

Several methods for expressing siRNA duplexes within cells from a construct to achieve long-term target gene suppression in cells are known in the art, e.g., including constructs that use a mammalian Pol III promoter system (e.g., H1 or U6/snRNA promoter systems (Tuschl, Nature Biotechnol., 20:440-448, 2002, which is incorporated in its entirety herein by reference) to express functional double-stranded siRNAs; (Bagella et al., J. Cell. Physiol., 177:206-213, 1998; Lee et al., Nature Biotechnol., 20:500-505, 2002; Paul et al., Nature Biotechnol., 20:505-508, 2002; Yu et al., Proc. Natl. Acad. Sci. U.S.A., 99(9):6047-6052, 2002; Sui et al., Proc. Natl. Acad. Sci. U.S.A. 99(6):5515-5520, 2002, each of which is incorporated in its entirety herein by reference). Transcriptional termination by RNA Pol III occurs at runs of four consecutive T residues in a DNA template, and can be used to provide a mechanism to end the siRNA transcript at a specific sequence. An siRNA is complementary to a sequence of a target gene in 5′-3′ and 3′-5′ orientations, and the two strands of a given siRNA can be expressed in the same construct or in separate constructs. Hairpin siRNAs, driven by H1 or U6 snRNA promoter and expressed in cells, can inhibit target gene expression (Bagella et al., 1998, supra; Lee et al., 2002, supra; Paul et al., 2002, supra; Yu et al., 2002, supra; Sui et al., 2002, supra).

In some embodiments, siRNAs of the present disclosure are double stranded nucleic acid duplexes (of, e.g., 15, 16, 17, 18, 19, 20, 21, 22, 23, 24, 25, 26, or 27 base pairs) comprising annealed complementary single stranded nucleic acid molecules. In some embodiments, siRNAs are short dsRNAs comprising annealed complementary single strand RNAs. In some embodiments, siRNAs comprise an annealed RNA:DNA duplex, wherein the sense strand of a duplex is a DNA molecule and the antisense strand of the same duplex is a RNA molecule.

In some embodiments, duplexed siRNAs comprise a 2 or 3 nucleotide 3′ overhang on each strand of a duplex. In some embodiments, siRNAs comprise 5′-phosphate and 3′-hydroxyl groups.

In some embodiments, a siRNA molecule of the present disclosure includes one or more natural nucleobase and/or one or more modified nucleobases derived from a natural nucleobase. Examples include, but are not limited to, uracil, thymine, adenine, cytosine, and guanine having their respective amino groups protected by acyl protecting groups, 2-fluorouracil, 2-fluorocytosine, 5-bromouracil, 5-iodouracil, 2,6-diaminopurine, azacytosine, pyrimidine analogs such as pseudoisocytosine and pseudouracil and other modified nucleobases such as 8-substituted purines, xanthine, or hypoxanthine (the latter two being natural degradation products). Exemplary modified nucleobases are disclosed in Chiu and Rana, R N A, 2003, 9, 1034-1048, Limbach et al. Nucleic Acids Research, 1994, 22, 2183-2196 and Revankar and Rao, Comprehensive Natural Products Chemistry, vol. 7, 313, each of which is incorporated in its entirety herein by reference.

Modified nucleobases also include expanded-size nucleobases in which one or more aryl rings, such as phenyl rings, have been added. Nucleic base replacements described in the Glen Research catalog (available on the world wide web at glenresearch.com); Krueger AT et al., Acc. Chem. Res., 2007, 40, 141-150; Kool, ET, Acc. Chem. Res., 2002, 35, 936-943; Benner S. A., et al., Nat. Rev. Genet., 2005, 6, 553-543; Romesberg, F. E., et al., Curr. Opin. Chem. Biol., 2003, 7, 723-733; Hirao, I., Curr. Opin. Chem. Biol., 2006, 10, 622-627, each of which is incorporated in its entirety herein by reference, are contemplated as useful for siRNA molecules described herein. In some embodiments, modified nucleobases also encompass structures that are not considered nucleobases but are other moieties such as, but not limited to, corrin- or porphyrin-derived rings. Porphyrin-derived base replacements have been described in Morales-Rojas, H and Kool, ET, Org. Lett., 2002, 4, 4377-4380, which is incorporated in its entirety herein by reference.

In some embodiments, modified nucleobases are of any one of the following structures, optionally substituted:

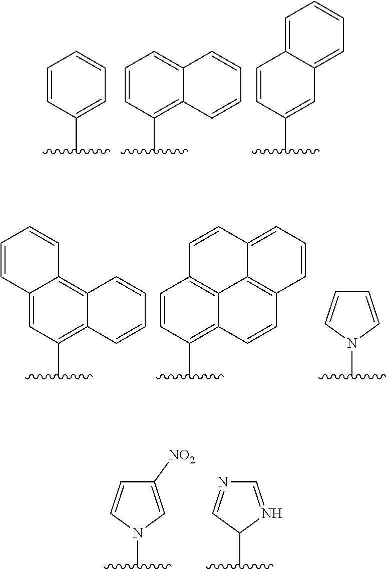

In some embodiments, a modified nucleobase is fluorescent. Exemplary such fluorescent modified nucleobases include phenanthrene, pyrene, stillbene, isoxanthine, isozanthopterin, terphenyl, terthiophene, benzoterthiophene, coumarin, lumazine, tethered stillbene, benzo-uracil, and naphtho-uracil, as shown below:

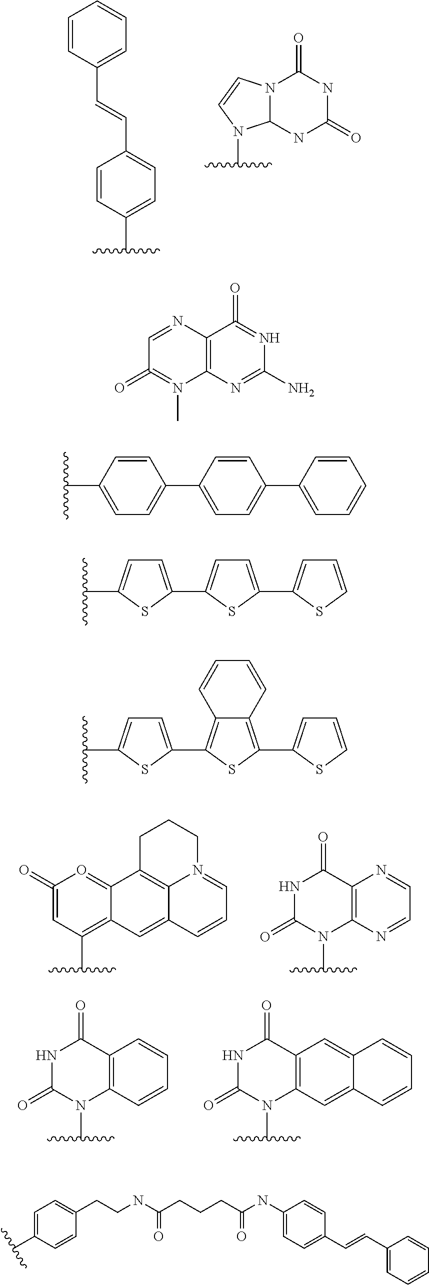

In some embodiments, a modified nucleobase is unsubstituted. In some embodiments, a modified nucleobase is substituted. In some embodiments, a modified nucleobase is substituted such that it contains, e.g., heteroatoms, alkyl groups, or linking moieties connected to fluorescent moieties, biotin or avidin moieties, or other protein or peptides. In some embodiments, a modified nucleobase is a “universal base” that is not a nucleobase in the most classical sense, but that functions similarly to a nucleobase. One representative example of such a universal base is 3-nitropyrrole.

In some embodiments, siRNA molecules described herein include nucleosides that incorporate modified nucleobases and/or nucleobases covalently bound to modified sugars. Some examples of nucleosides that incorporate modified nucleobases include 4-acetylcytidine; 5-(carboxyhydroxylmethyl)uridine; 2′-O-methylcytidine; 5-carboxymethylaminomethyl-2-thiouridine; 5-carboxymethylaminomethyluridine; dihydrouridine; 2′-O-methylpseudouridine; beta,D-galactosylqueosine; 2′-O-methylguanosine; N⁶-isopentenyladenosine; 1-methyladenosine; 1-methylpseudouridine; 1-methylguanosine; 1-methylinosine; 2,2-dimethylguanosine; 2-methyladenosine; 2-methylguanosine; N⁷-methylguanosine; 3-methyl-cytidine; 5-methylcytidine; 5-hydroxymethylcytidine; 5-formylcytosine; 5-carboxylcytosine; N⁶-methyladenosine; 7-methylguanosine; 5-methylaminoethyluridine; 5-methoxyaminomethyl-2-thiouridine; beta,D-mannosylqueosine; 5-methoxycarbonylmethyluridine; 5-methoxyuridine; 2-methylthio-N⁶-isopentenyladenosine; N-((9-beta,D-ribofuranosyl-2-methylthiopurine-6-yl)carbamoyl)threonine; N-((9-beta,D-ribofuranosylpurine-6-yl)-N-methylcarbamoyl)threonine; uridine-5-oxyacetic acid methylester; uridine-5-oxyacetic acid (v); pseudouridine; queosine; 2-thiocytidine; 5-methyl-2-thiouridine; 2-thiouridine; 4-thiouridine; 5-methyluridine; 2′-O-methyl-5-methyluridine; and 2′-O-methyluridine.

In some embodiments, nucleosides include 6′-modified bicyclic nucleoside analogs that have either (R) or (S)-chirality at the 6′-position and include the analogs described in U.S. Pat. No. 7,399,845, which is incorporated in its entirety herein by reference. In other embodiments, nucleosides include 5′-modified bicyclic nucleoside analogs that have either (R) or (S)-chirality at the 5′-position and include the analogs described in U.S. Publ. No. 20070287831, which is incorporated in its entirety herein by reference. In some embodiments, a nucleobase or modified nucleobase is 5-bromouracil, 5-iodouracil, or 2,6-diaminopurine. In some embodiments, a nucleobase or modified nucleobase is modified by substitution with a fluorescent moiety.

Methods of preparing modified nucleobases are described in, e.g., U.S. Pat. Nos. 3,687,808; 4,845,205; 5,130,30; 5,134,066; 5,175,273; 5,367,066; 5,432,272; 5,457,187; 5,457,191; 5,459,255; 5,484,908; 5,502,177; 5,525,711; 5,552,540; 5,587,469; 5,594,121, 5,596,091; 5,614,617; 5,681,941; 5,750,692; 6,015,886; 6,147,200; 6,166,197; 6,222,025; 6,235,887; 6,380,368; 6,528,640; 6,639,062; 6,617,438; 7,045,610; 7,427,672; and 7,495,088, each of which is incorporated in its entirety herein by reference.

In some embodiments, a siRNA molecule described herein includes one or more modified nucleotides wherein a phosphate group or linkage phosphorus in its nucleotides are linked to various positions of a sugar or modified sugar. As non-limiting examples, a phosphate group or linkage phosphorus can be linked to a 2′, 3′, 4′ or 5′ hydroxyl moiety of a sugar or modified sugar. Nucleotides that incorporate modified nucleobases as described herein are also contemplated in this context.

Other modified sugars can also be incorporated within a siRNA molecule. In some embodiments, a modified sugar contains one or more substituents at a 2′ position including one of the following: —F; —CF₃, —CN, —N₃, —NO, —NO₂, —OR′, —SR′, or —N(R′)₂, wherein each R′ is independently as defined above and described herein; —O—(C₁-C₁₀ alkyl), —S—(C₁-C₁₀ alkyl), —NH—(C₁-C₁₀ alkyl), or —N(C₁-C₁₀ alkyl)₂; —O—(C₂-C₁₀ alkenyl), —S—(C₂-C₁₀ alkenyl), —NH—(C₂-C₁₀ alkenyl), or —N(C₂-C₁₀ alkenyl)₂; —O—(C₂-C₁₀ alkynyl), —S—(C₂-C₁₀ alkynyl), —NH—(C₂-C₁₀ alkynyl), or —N(C₂-C₁₀ alkynyl)₂; or —O—(C₁-C₁₀ alkylene)—O—(C₁-C₁₀ alkyl), —O—(C₁-C₁₀ alkylene)—NH—(C₁-C₁₀ alkyl) or —O—(C₁-C₁₀ alkylene)—NH(C₁-C₁₀ alkyl)₂, —NH— alkylene)—O—(C₁-C₁₀ alkyl), or —N(C₁-C₁₀ alkylene)—O—(C₁-C₁₀ alkyl), wherein the alkyl, alkylene, alkenyl and alkynyl may be substituted or unsubstituted. Examples of substituents include, and are not limited to, —O(CH₂)ₙOCH₃, and —O(CH₂)ₙNH₂, wherein n is from 1 to about 10, MOE, DMAOE, DMAEOE. Also contemplated herein are modified sugars described in WO 2001/088198; and Martin et al., Helv. Chim. Acta, 1995, 78, 486-504, each of which is incorporated in its entirety herein by reference. In some embodiments, a modified sugar comprises one or more groups selected from a substituted silyl group, an RNA cleaving group, a reporter group, a fluorescent label, an intercalator, a group for improving pharmacokinetic properties of a nucleic acid, a group for improving pharmacodynamic properties of a nucleic acid, or other substituents having similar properties. In some embodiments, modifications are made at one or more of a 2′, 3′, 4′, 5′, or 6′ positions of a sugar or modified sugar, including a 3′ position of a sugar on a 3′-terminal nucleotide or in a 5′ position of a 5′-terminal nucleotide.

In some embodiments, a 2′-OH of a ribose is replaced with a substituent including one of the following: —H, —CF₃, —CN, —N₃, —NO, —NO₂, —OR′, —SR′, or —N(R′)₂, wherein each R′ is independently as defined above and described herein; —O—(C₁-C₁₀ alkyl), —S—(C₁-C₁₀ alkyl), —NH—(C₁-C₁₀ alkyl), or —N(C₁-C₁₀ alkyl)₂; —O—(C₂-C₁₀ alkenyl), —S—(C₂-C₁₀ alkenyl), —NH—(C₂-C₁₀ alkenyl), or —N(C₂-C₁₀ alkenyl)₂; —O—(C₂-C₁₀ alkynyl), —S—(C₂-C₁₀ alkynyl), —NH—(C₂-C₁₀ alkynyl), or —N(C₂-C₁₀ alkynyl)₂; or —O—(C₁-C₁₀ alkylene)—O—(C₁-C₁₀ alkyl), —O—(C₁-C₁₀ alkylene)—NH—(C₁-C₁₀ alkyl) or —O—(C₁-C₁₀ alkylene)—NH(C₁-C₁₀ alkyl)₂, —NH— alkylene)—O—(C₁-C₁₀ alkyl), or —N(C₁-C₁₀) alkyl)—(C₁-C₁₀ alkylene)—O—(C₁-C₁₀ alkyl), wherein an alkyl, alkylene, alkenyl and alkynyl may be substituted or unsubstituted. In some embodiments, a 2′-OH is replaced with —H (deoxyribose). In some embodiments, a 2′-OH is replaced with —F. In some embodiments, a 2′-OH is replaced with —OR′. In some embodiments, a 2′-OH is replaced with —OMe. In some embodiments, a 2′-OH is replaced with —OCH₂CH₂OMe.

Modified sugars also include locked nucleic acids (LNAs). In some embodiments, a locked nucleic acid has the structure indicated below. A locked nucleic acid of the structure below is indicated, wherein Ba represents a nucleobase or modified nucleobase as described herein, and wherein R^{2s} is —OCH₂C4′—

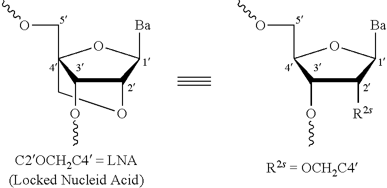

In some embodiments, a modified sugar is an ENA such as those described in, e.g., Seth et al., J Am Chem Soc. 2010 Oct. 27; 132(42): 14942-14950, which is incorporated in its entirety herein by reference. In some embodiments, a modified sugar is any of those found in an XNA (xenonucleic acid), for instance, arabinose, anhydrohexitol, threose, 2′fluoroarabinose, or cyclohexene.

Modified sugars include sugar mimetics such as cyclobutyl or cyclopentyl moieties in place of the pentofuranosyl sugar (see, e.g., U.S. Pat. Nos. 4,981,957; 5,118,800; 5,319,080; and 5,359,044, each of which is incorporated in its entirety herein by reference). Some modified sugars that are contemplated include sugars in which an oxygen atom within a ribose ring is replaced by nitrogen, sulfur, selenium, or carbon. In some embodiments, a modified sugar is a modified ribose wherein an oxygen atom within a ribose ring is replaced with nitrogen, and wherein a nitrogen is optionally substituted with an alkyl group (e.g., methyl, ethyl, isopropyl, etc.).

Non-limiting examples of modified sugars include glycerol, which form glycerol nucleic acid (GNA) analogues. An exemplary GNA analogue is described in Zhang, R et al., J. Am. Chem. Soc., 2008, 130, 5846-5847, which is incorporated in its entirety herein by reference; see also Zhang L, et al., J. Am. Chem. Soc., 2005, 127, 4174-4175 and Tsai CH et al., PNAS, 2007, 14598-14603, each which is incorporated in its entirety herein by reference. Another example of a GNA derived analogue, flexible nucleic acid (FNA) based on mixed acetal aminal of formyl glycerol, is described in each of Joyce GF et al., PNAS, 1987, 84, 4398-4402 and Heuberger BD and Switzer C, J. Am. Chem. Soc., 2008, 130, 412-413, each of which is incorporated in its entirety herein by reference. Additional non-limiting examples of modified sugars include hexopyranosyl (6′ to 4′), pentopyranosyl (4′ to 2′), pentopyranosyl (4′ to 3′), or tetrofuranosyl (3′ to 2′) sugars.

Modified sugars and sugar mimetics can be prepared by methods known in the art, including, but not limited to: A. Eschenmoser, Science (1999), 284:2118; M. Bohringer et al., Helv. Chim. Acta (1992), 75:1416-1477; M. Egli et al., J. Am. Chem. Soc. (2006), 128(33):10847-56; A. Eschenmoser in Chemical Synthesis: Gnosis to Prognosis, C. Chatgilialoglu and V. Sniekus, Ed., (Kluwer Academic, Netherlands, 1996), p. 293; K.-U. Schoning et al., Science (2000), 290:1347-1351; A. Eschenmoser et al., Helv. Chim. Acta (1992), 75:218; J. Hunziker et al., Helv. Chim. Acta (1993), 76:259; G. Otting et al., Helv. Chim. Acta (1993), 76:2701; K. Groebke et al., Helv. Chim. Acta (1998), 81:375; and A. Eschenmoser, Science (1999), 284:2118. Modifications to 2′ modifications can be found in Verma, S. et al. Annu. Rev. Biochem. 1998, 67, 99-134 and all references therein, each of which is incorporated in its entirety herein by reference. Specific modifications to a ribose can be found in the following references: 2′-fluoro (Kawasaki et. al., J. Med. Chem., 1993, 36, 831-841), 2′-MOE (Martin, P. Helv. Chim. Acta 1996, 79, 1930-1938), “LNA” (Wengel, J. Acc. Chem. Res. 1999, 32, 301-310); PCT Publication No. WO2012/030683, each of which is incorporated in its entirety herein by reference.

In some embodiments, a siRNA described herein can be introduced to a target cell as an annealed duplex siRNA. In some embodiments, a siRNA described herein is introduced to a target cell as single stranded sense and antisense nucleic acid sequences that, once within a target cell, anneal to form a siRNA duplex. Alternatively, sense and antisense strands of an siRNA can be encoded by an expression construct (such as an expression construct described herein) that is introduced to a target cell. Upon expression within a target cell, transcribed sense and antisense strands can anneal to reconstitute an siRNA.

In some embodiments, an siRNA molecule as described herein can be synthesized by standard methods known in the art, e.g., by use of an automated synthesizer. Without being bound by any particular theory, RNAs produced by such methodologies tend to be highly pure and to anneal efficiently to form siRNA duplexes. In some embodiments, following chemical synthesis, single stranded RNA molecules can be deprotected, annealed to form siRNAs, and purified (e.g., by gel electrophoresis or HPLC). Alternatively, in some embodiments, standard procedures can be used for in vitro transcription of RNA from DNA templates, e.g., carrying one or more RNA polymerase promoter sequences (e.g., T7 or SP6 RNA polymerase promoter sequences). Protocols for preparation of siRNAs using T7 RNA polymerase are known in the art (see, e.g., Donze and Picard, Nucleic Acids Res. 2002; 30:e46; and Yu et al., Proc. Natl. Acad. Sci. USA 2002; 99:6047-6052, each of which is incorporated in its entirety herein by reference). In some embodiments, sense and antisense transcripts can be synthesized in two independent reactions and annealed later. In some embodiments, sense and antisense transcripts can be synthesized simultaneously in a single reaction.

In some embodiments, an siRNA molecule can also be formed within a cell by transcription of RNA from an expression construct introduced into a cell (see, e.g., Yu et al., Proc. Natl. Acad. Sci. USA 2002; 99:6047-6052, which is incorporated in its entirety herein by reference). For example, in some embodiments, an expression construct for in vivo production of siRNA molecules can include one or more siRNA encoding sequences operably linked to elements necessary for proper transcription of an siRNA encoding sequence(s), including, e.g., promoter elements and transcription termination signals. In some embodiments, preferred promoters for use in such expression constructs may include, e.g., a polymerase-III promoter, e.g., a polymerase-III HI-RNA promoter (see, e.g., Brummelkamp et al., Science 2002; 296:550-553, which is incorporated in its entirety herein by reference), a U6 polymerase-III promoter (see, e.g., Sui et al., Proc. Natl. Acad. Sci. USA 2002; Paul et al., Nature Biotechnol. 2002; 20:505-508; and Yu et al., Proc. Natl. Acad. Sci. USA 2002; 99:6047-6052, each of which is incorporated in its entirety herein by reference). In some embodiments, an siRNA expression construct can comprise one or more construct sequences that facilitate cloning of an expression construct. Standard constructs that can be used include, e.g., pSilencer 2.0-U6 construct (Ambion Inc., Austin, Tex.).

. In some embodiments, an siRNA is or comprises nucleotides of any one of SEQ ID Nos: 48-55. In some embodiments, an siRNA comprises a mature guide strand having a nucleotide sequence that is at least 90%, 91%, 92%, 93%, 94%, 95%, 96%, 97%, 98%, 99%, or 100% identical to any one of SEQ ID NOs:48-55 (or a portion thereof). In some embodiments, a portion is 15, 16, 17, 18, 19, or 20 nucleotides long. In some embodiments, an siRNA comprises a mature guide strand having a nucleotide sequence that is 100% identical to nucleotides 2-8 of any one of SEQ ID NOs: 48-55

In some embodiments, the present disclosure provides shRNA sequences, which, when introduced into a cell will be cleaved to siRNAs. Accordingly, by way of non-limiting example, shRNA sequences of the present disclosure may be or comprise those provided in SEQ ID NOs 48-55 or elsewhere herein the disclosure.

Each of the following shRNA sequences is provided in 5′ to 3′ order:

(SEQ ID NO: 48)
TCCTCATCTTGGAATTCGTGATGCGAACATCACGAATTCCAAGATGA
GGA;
(SEQ ID NO: 49)
CGCCTTACTGGGCATCTCTTTCTCGAAAGAAAGAGATGCCCAGTAAG
GCG-;
(SEQ ID No: 50)
CTGGTACTACTATGACAGTATCGAAATACTGTCATAGTAGTACCAG;
(SEQ ID NO: 51)
AAGTAGCAGAGGAGAAGAGCTCGAAAGCTCTTCTCCTCTGCTACTT;
(SEQ ID NO: 52)
AAGACAGTCATCCGCTCCATCCGAAGATGGAGCGGATGACTGTCTT;
(SEQ ID NO: 53)
AAGGACGTCATTGAGCAGTACCGAAGTACTGCTCAATGACGTCCTT;
(SEQ ID NO: 54)
GCCGGATCAAGAGCCTGCAAACGAATTTGCAGGCTCTTGATCCGGC;
(SEQ ID NO: 55)
GGTGGATGAAATCAGCATGATCGAAATCATGCTGATTTCATCCACC;

miRNA

The present disclosure provides technologies related to or comprising one or more inhibitory nucleic acid molecules such as, e.g., one or more nucleotide sequences that are, comprise, or encode, microRNAs. MicroRNAs (miRNAs) are a highly conserved class of small RNA molecules that are transcribed from DNA in genomes of plants and animals, but are not translated into protein. As is known to those in the art, animal cells express a range of noncoding RNAs of approximately 22 nucleotides termed micro RNA (miRNAs) and can regulate gene expression at a post transcriptional or translational level during animal development. miRNAs are excised from an approximately 70 nucleotide precursor RNA stem-loop. By substituting stem sequences of an miRNA precursor with miRNA sequence complementary to a target mRNA, a construct that expresses a novel miRNA can be used to produce siRNAs to initiate RNAi against specific mRNA targets in mammalian cells (Zeng, Mol. Cell, 9:1327-1333, 2002). In some embodiments, when expressed by DNA constructs containing polymerase III promoters, micro-RNA designed hairpins can silence gene expression (McManus, RNA 8:842-850, 2002).

In some embodiments, miRNAs can be synthesized and locally or systemically administered to a subject, e.g., for therapeutic purposes. In some embodiments, miRNAs can be designed and/or synthesized as mature molecules or precursors (e.g., pri- or pre-miRNAs). In some embodiments, a pre-miRNA includes a guide strand and a passenger strand that are the same length (e.g., about 15, 16, 17, 18, 19, 20, 21, 22, 23, 24, or 25 nucleotides). In some embodiments, a pre-miRNA includes a guide strand and a passenger strand that are different lengths (e.g., one strand is about 19 nucleotides, and the other is about 21 nucleotides). In some embodiments, an miRNA can target a coding region, a 5′ untranslated region, and/or a 3′ untranslated region, of endogenous mRNA. In some embodiments, an miRNA comprises a guide strand comprising a nucleotide sequence having sufficient sequence complementary with an endogenous mRNA of a subject to hybridize with and inhibit expression of endogenous mRNA.

In some embodiments, miRNAs has advantages compared to shRNAs for inhibiting nucleic acids. For example, shRNA requires a high level of expression, can clog Argonaut machinery, is not endogenous, and relies on two promoters. By contrast, in some embodiments, it is contemplated that miRNA is more “endogenous” than shRNA, and therefore, is expressed at similar levels as KCNQ4 driven by a single promoter. That is, miRNAs can be synthetic or naturally occurring and naturally-occurring miRNAs are present in cells across vertebrate and invertebrate species. In contrast, to date, shRNAs have not been detected as a naturally occurring component of a cell. In some embodiments, miRNAs have low or no passenger strand generation, can be used therapeutically, and/or are expressed in neurons and hair cells.

The present disclosure describes that, in some embodiments, minor changes (e.g., one or more substitutions) were made to the passenger strand of a given mRNA. In some such embodiments, such change(s) (e.g., substitution(s)) were made in order to ensure that the synthetic KCNQ4 miRNA would have the same secondary structure as an endogenous miRNA gene. For example, in some constructs, the G at position 73 of the KCNQ miR1 hsa-3miR-335 construct can be substituted for a C to generate a bulge (e.g., Pyr:Pur (G:U) can be substituted for Pyr:Pyr (C:U) base pairing). One or more such base changes (e.g., substitution) can, in some embodiments, result in a secondary structure (e.g., bulge position) in the synthetic miRNA such that the secondary structure is similar or identical to that of an endogenous miRNA gene. One of ordinary skill in the art will appreciate that the exact location (e.g., base or bases) where one or more changes (e.g., substitutions) is made will vary with the exact sequence of a synthetic miRNA, and one of such skill will understand how to modify bases relative to the sequence of a synthesized construct as compared to its endogenous counterpart.

The present disclosure also recognizes the surprising finding that, in some embodiments, an miRNA of the same sequence of an shRNA provides an unexpected increase in efficiency. For example, in some embodiments, if an shRNA and miRNA are designed for a given target, the present disclosure provides the surprising finding that an miRNA is much more efficient (as measured by KD) as compared to an shRNA of the same sequence. In some embodiments, an miRNA comprises a mature guide strand having a nucleotide sequence that is at least 90%, 91%, 92%, 93%, 94%, 95%, 96%, 97%, 98%, 99%, or 100% identical to any one of SEQ ID NOs:56-70(or a portion thereof). In some embodiments, a portion is 15, 16, 17, 18, 19, or 20 nucleotides long. In some embodiments, an miRNA comprises a mature guide strand having a nucleotide sequence that is 100% identical to nucleotides 2-8 of any one of SEQ ID NOs: 56-70.

Each of the following miR sequences is provided in 5′ to 3′ order:

miR1-155
(SEQ ID NO: 56)
TTGAGAATCCTGATGGAGCGG
miR2-155
(SEQ ID NO: 57)
TTGAGTCTCAGAGATGCCC
miR4-155
(SEQ ID NO: 58)
TTCTCGAAGTGCTTCTGCCGG
miR5-155
(SEQ ID NO: 59)
TTCTCCACCTTGACCACGCGT
miR6-155
(SEQ ID NO: 60)
ATACTGTCATAGTAGTAGCAG
miR7-155
(SEQ ID NO: 61)
TAGTCGGAGGTGATGTCGGGG
miR1-16
(SEQ ID NO: 62)
TTGAGAATCCTGATGGAGCGG
miR1-26
(SEQ ID NO: 63)
TTGAGAATCCTGATGGAGCGG
miR1-96
(SEQ ID NO: 64)
TTGAGAATCCTGATGGAGCGG
miR1-122
(SEQ ID NO: 65)
TTGAGAATCCTGATGGAGCGG
miR1-135
(SEQ ID NO: 66)
TTGAGAATCCTGATGGAGCGG
miR1-182
(SEQ ID NO: 67)
TTGAGAATCCTGATGGAGCGG
miR1-183
(SEQ ID NO: 68)
TTGAGAATCCTGATGGAGCGG
miR1-335
(SEQ ID NO: 69)
TTGAGAATCCTGATGGAGCGG
miR1-451
(SEQ ID NO: 70)
TTGAGAATCCTGATGGAGCGG
mouse miR2-26
(SEQ ID NO: 96)
TTGAGTCTTAGAGAGGCCCGG
mouse miR6-26
(SEQ ID NO: 97)
ATGCTGTCATAGTAATACCAG

In some embodiments, the present disclosure provides methods for choosing miRNAs targeting sequences expressed within a variety of compartments within an ear. For example, spiral ganglion: miR-96, -182, -183, -15a, -30b, -99a, -18a, -124a, -194; spiral limbus: miR-96, -182, -193, -205; Reissner's membrane: miR-205; marginal cells: miR-376a, -376b; spiral ligament: miR-205; supporting cells: miR-15a, -30b, -99a; hair cells: miR-96, -182, -183, -15a, -30b, -99a, -18a, -140, 194; basilar membrane: miR-205, -15a, -30b, -99a; inner sulcus: miR-96, -182, -183). Ushakov et al., 2013. As described herein, miRNA scaffolds miR-16, miR-26, miR-96 (hair cells), miR-122, miR-135, miR-155, miR-182 (hair cells), miR-183 (hair cells), miR-335, and miR-451 were considered for initial testing of impact on KCNQ4 expression. In some embodiments, an miRNA may comprise or consist of miR1-155; miR2-155; miR4-155; miR5-155; miR6-155; miR7-155; miR1-16; miR1-26; miR1-96; miR1-122; miR1-135; miR1-182; miR1-183; miR1-335; miR1-451. In some embodiments, a construct of the present disclosure may comprise one or more miRNAs selected from miR1-155; miR2-155; miR4-155; miR5-155; miR6-155; miR7-166 miR1-16; miR1-26; miR1-96; miR1-122; miR1-135; miR1-182; miR1-183; miR1-335; miR1-451 and combinations thereof.

Antisense Nucleic Acid

In some embodiments, an inhibitory nucleic acid molecule may be or comprise an antisense nucleic acid molecule, e.g., nucleic acid molecules whose nucleotide sequence is complementary to all or part of an mRNA encoding a potassium channel (e.g., KCNQ4) protein. In some embodiments, an antisense nucleic acid molecule can be antisense to all or part of a non-coding region of a coding strand of a nucleotide sequence encoding a potassium channel (e.g., KCNQ4) protein. In some embodiments, a non-coding regions (“5′ and 3′ untranslated regions”) are 5′ and 3′ sequences that flank a coding region and are not translated into amino acids. Based upon sequences disclosed herein, one of skill in the art can easily choose and synthesize any of a number of appropriate antisense molecules to target a potassium channel (e.g., KCNQ4) gene described herein. For example, a “gene walk” comprising a series of oligonucleotides of 15-30 nucleotides spanning a length of a nucleic acid (e.g., a potassium channel (e.g., KCNQ4) mRNA) can be prepared, followed by testing for inhibition of expression of to gene. Optionally, gaps of 5-10 nucleotides can be left between oligonucleotides to reduce numbers of oligonucleotides synthesized and tested.

In some embodiments, an antisense oligonucleotide can be, for example, about 5, 10, 15, 20, 25, 30, 35, 40, 45, or 50 nucleotides or more in length. One of skill in the art will recognize that an antisense oligonucleotide can be synthesized using various different chemistries.

Ribozymes

In some embodiments, an inhibitory nucleic acid molecule may be or comprise a ribozyme. As is known to those of skill in the art, ribozymes are catalytic RNA molecules with ribonuclease activity. In some embodiments, a ribozyme may be used as a controllable promoter. In some embodiments, ribozymes are capable of cleaving a single-stranded nucleic acid, such as an mRNA, to which they have a complementary region. Thus, in some embodiments, ribozymes (e.g., hammerhead ribozymes (described in Haselhoff and Gerlach, Nature, 334:585-591, 1988, which is incorporated in its entirety herein by reference)) can be used to catalytically cleave mRNA transcripts to thereby inhibit translation of a protein encoded by a given mRNA. Methods of designing and producing ribozymes are known in the art (see, e.g., Scanlon, 1999, Therapeutic Applications of Ribozymes, Humana Press, which is incorporated in its entirety herein by reference). In some embodiments, for example, a ribozyme having specificity for a KCNQ4 gene product mRNA can be designed based upon nucleotide sequence of a KCNQ4 gene product cDNA (e.g., any exemplary cDNA sequences described herein). For example, a derivative of a Tetrahymena L-19 IVS RNA can be constructed in which nucleotide sequence of an active site is complementary to a nucleotide sequence to be cleaved in a KCNQ4 gene product mRNA (Cech et al. U.S. Pat. No. 4,987,071; and Cech et al., U.S. Pat. No. 5,116,742, each of which is incorporated in its entirety herein by reference). Alternatively, an mRNA encoding a KCNQ4 gene product protein can be used to select a catalytic RNA having a specific ribonuclease activity from a pool of RNA molecules (See, e.g., Bartel and Szostak, Science, 261:1411-1418, 1993, which is incorporated in its entirety herein by reference).

g. Pharmaceutical Compositions and Kits

Pharmaceutical compositions of the present disclosure may include constructs, as described herein. For example, in some embodiments, pharmaceutical compositions may comprise AAV constructs and/or AAV particles. In some such embodiments, such AAV particles comprise one or more constructs, which comprise a nucleic acid, e.g., one or a plurality of AAV constructs. For example, a pharmaceutical composition of the present disclosure comprise as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose, or dextrans; mannitol; proteins; polypeptides or amino acids such as glycine; antioxidants; chelating agents such as EDTA or glutathione; adjuvants (e.g., aluminum hydroxide); and preservatives. In some embodiments, compositions of the present disclosure are formulated for intravenous administration. In some embodiments compositions of the present disclosure are formulated for intra-cochlear administration.

In some embodiments, therapeutic compositions of the present disclosure are formulated for intra-cochlear administration. In some embodiments, a therapeutic composition is formulated to comprise a lipid nanoparticle, a polymeric nanoparticle, a mini-circle DNA or a CELiD DNA.

In some embodiments, a therapeutic composition is formulated to comprise a synthetic perilymph solution. For example, in some embodiments, a synthetic perilymph solution includes 20-200 mM NaCl; 1-5 mM KCl; 0.1-10 mM CaCl₂; 1-10 mM glucose; and 2-50 mM HEPES, with a pH between about 6 and about 9. In some embodiments, any of the pharmaceutical compositions described herein may further comprise one or more agents that promote entry of a nucleic acid or any of the constructs described herein into a mammalian cell (e.g., a liposome or cationic lipid). In some embodiments, any constructs described herein can be formulated using natural and/or synthetic polymers. Non-limiting examples of polymers that may be included in any compositions described herein can include, but are not limited to, DYNAMIC POLYCONJUGATE® (Arrowhead Research Corp., Pasadena, Calif.), formulations from Minis Bio (Madison, Wis.) and Roche Madison (Madison, Wis.), PhaseRX polymer formulations such as, without limitation, SMARTT POLYMER TECHNOLOGY® (PhaseRX, Seattle, Wash.), DMRI/DOPE, poloxamer, VAXFECTIN® adjuvant from Vical (San Diego, Calif.), chitosan, cyclodextrin from Calando Pharmaceuticals (Pasadena, Calif.), dendrimers and poly (lactic-co-glycolic acid) (PLGA) polymers, RONDEL™ (RNAi/Oligonucleotide Nanoparticle Delivery) polymers (Arrowhead Research Corporation, Pasadena, Calif.), and pH responsive co-block polymers, such as, but not limited to, those produced by PhaseRX (Seattle, Wash.). Many of these polymers have demonstrated efficacy in delivering oligonucleotides in vivo into a mammalian cell (see, e.g., deFougerolles, Human Gene Ther. 19:125-132, 2008; Rozema et al., Proc. Natl. Acad. Sci. U.S.A. 104:12982-12887, 2007; Rozema et al., Proc. Natl. Acad. Sci. U.S.A. 104:12982-12887, 2007; Hu-Lieskovan et al., Cancer Res. 65:8984-8982, 2005; Heidel et al., Proc. Natl. Acad. Sci. U.S.A. 104:5715-5721, 2007, each of which is incorporated in its entirety herein by reference). Any compositions described herein can be, e.g., a pharmaceutical composition.

In some embodiments, a composition includes a pharmaceutically acceptable carrier (e.g., phosphate buffered saline, saline, or bacteriostatic water). Upon formulation, solutions can be administered in a manner compatible with a dosage formulation and in such amount as is therapeutically effective. Formulations are easily administered in a variety of dosage forms such as injectable solutions, injectable gels, drug-release capsules, and the like.

Compositions provided herein can be, e.g., formulated to be compatible with their intended route of administration. A non-limiting example of an intended route of administration is local administration (e.g., intra-cochlear administration).

Also provided are kits including any compositions described herein. In some embodiments, a kit can include a solid composition (e.g., a lyophilized composition including at least one construct as described herein) and a liquid for solubilizing a lyophilized composition.

In some embodiments, a kit can include a pre-loaded syringe including any compositions described herein.

In some embodiments, a kit includes a vial comprising any of the compositions described herein (e.g., formulated as an aqueous composition, e.g., an aqueous pharmaceutical composition).

In some embodiments, a kit can include instructions for performing any methods described herein.

h. Cells

In some embodiments, the present disclosure provides a cell (e.g., a mammalian cell, e.g., a human cell, e.g., an ear hair cell, e.g., an OHC, an IHC, etc.) that comprises any nucleic acids, constructs (e.g., at least two different constructs described herein), compositions, etc., as described herein. As will be appreciated by one of skill in the art, nucleic acids and constructs described herein can be introduced into any cell (e.g., a mammalian cell, e.g., a human cell, e.g., an ear hair cell, e.g., an OHC, an IHC, etc.). Non-limiting examples of certain constructs and methods for introducing constructs into cells are described herein.

In some embodiments, a cell is a human cell, a mouse cell, a porcine cell, a rabbit cell, a dog cell, a rat cell, a sheep cell, a cat cell, a horse cell, a non-human primate cell, or an insect cell. In some embodiments, a cell is a specialized cell of the cochlea. In some embodiments, a cell is a cochlear inner hair cell or a cochlear outer hair cell. In some embodiments, a cell is a cochlear inner hair cell. In some embodiments, a cell is a cochlear outer hair cell.

In some embodiments, a cell is in vitro. In some embodiments, a cell is in vivo or ex vivo. For example, in some embodiments, cell is present in a mammal. In some embodiments, a cell (e.g., a mammalian cell) is autologous cell obtained, e.g., from a subject (e.g., a mammal) and cultured ex vivo.

In some embodiments, cells provided by the present disclosure are transfected host cells. In some embodiments, transfection is used to refer to uptake of foreign DNA by a cell, and a cell has been “transfected” when exogenous DNA has been introduced inside a cell membrane. A number of transfection techniques are generally known in the art (see, e.g., Graham et al. (1973) Virology, 52:456; Sambrook et al. (1989) Molecular Cloning, a laboratory manual, Cold Spring Harbor Laboratories, New York, Davis et al. (1986) Basic Methods in Molecular Biology, Elsevier; and Chu et al. (1981) Gene 13:197, each of which is incorporated in its entirety herein by reference). Such techniques can be used to introduce one or more exogenous nucleic acids, such as a nucleotide integration construct and other nucleic acid molecules, into suitable host cells.

In some embodiments, a host cell is a mammalian cell. A host cell may be used as a recipient of an AAV helper construct, an AAV minigene plasmid, an accessory function construct, and/or other transfer DNA associated with production of recombinant AAVs. The term includes progeny of an original cell that has been transfected. In some embodiments, a host cell is a cell that has been transfected with an exogenous DNA sequence. It is understood that progeny of a single parental cell may not necessarily be completely identical in morphology or in genomic or total DNA complement as the original parent, due to natural, accidental, or deliberate mutation.

### 4. Methods

Among other things, the present disclosure provides methods. In some embodiments, a method comprises introducing a composition as described herein into the inner ear (e.g., a cochlea) of a subject. For example, provided herein are methods that in some embodiments include administering to an inner ear (e.g., cochlea) of a subject (e.g., an animal, e.g., a mammal, e.g., a primate, e.g., a human) a therapeutically effective amount of any composition described herein.

In some embodiments, the present disclosure provides methods of generating and/or testing one or more compositions of components thereof. In some such embodiments, compositions may be used to treat a subject with hearing loss. In some embodiments, the present disclosure provides methods of genetically modifying one or more cells. In some embodiments, methods of the present disclosure, including compositions produced or administered using such methods include those in WO 2019/028246 A2, PCT/US2019/060324, and PCT/US2019/060328, each of which is incorporated by reference herein, in its entirety.

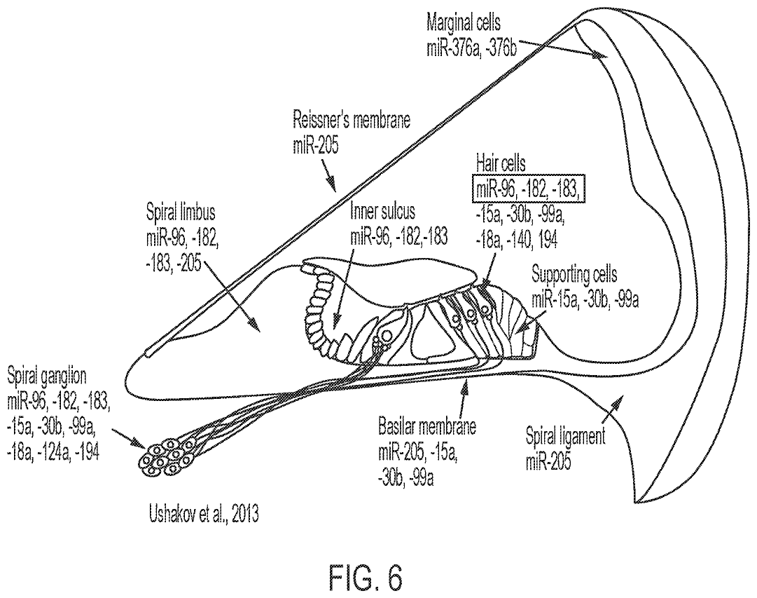

For example, in some embodiments, in addition to the exemplary compositions described herein, FIG. 6 depicts exemplary miRNA constructs that can be used in accordance with the present disclosure. These sequences and constructs can be tested (e.g., via double transfection) in cells (e.g., human cells, e.g., HEK cells, e.g., hair cells, e.g., outer hair cells), in single, and/or in multiplexed format. mRNA and protein levels are or can be measured using appropriate qualitative or quantitative techniques such as PCR, immunofluorescence (of cells) and western blot techniques. As described herein, exemplary miRNA constructs can be delivered as plasmids (or constructs), or via AAV compositions described herein.

a. Methods of Making

In some embodiments, viral constructs are prepared by any methods known to one of skill in the art. For example, in some embodiments, viral constructs are prepared using a standard triple transfection system (e.g., three plasmids/constructs, comprising (i) rep/cap genes, (ii) helper genes, and (iii) payloads (e.g., miRNA, KCNQ4, etc.) respectively, e.g., four plasmids/constructs, etc.) followed by standard isolation and purification methods (e.g., CsCl gradient). In some such embodiments, such viral preparations are formulated for delivery into a subject.

The present disclosure provides, among other things, method of making AAV-based constructs. In some embodiments, such methods include use of host cells. In some embodiments, a host cell is a mammalian cell. A host cell may be used as a recipient of an AAV helper construct, an AAV minigene plasmid, an accessory function construct, and/or other transfer DNA associated with production of recombinant AAVs. The term includes progeny of an original cell that has been transfected. Thus, a “host cell” as used herein may refer to a cell that has been transfected with an exogenous DNA sequence. It is understood that progeny of a single parental cell may not necessarily be completely identical in morphology or in genomic or total DNA complement as the original parent, due to natural, accidental, or deliberate mutation.

Additional methods for generating and isolating AAV viral constructs suitable for delivery to a subject are described in, e.g., U.S. Pat. Nos. 7,790,449; 7,282,199; WO 2003/042397; WO 2005/033321, WO 2006/110689; and U.S. Pat. No. 7,588,772, each of which is incorporated in its entirety herein by reference. In one system, a producer cell line is transiently transfected with a construct that encodes a transgene flanked by ITRs and a construct(s) that encodes rep and cap. In another system, a packaging cell line that stably supplies rep and cap is transiently transfected with a construct encoding a transgene flanked by ITRs. In each of these systems, AAV virions are produced in response to infection with helper adenovirus or herpesvirus, and AAVs are separated from contaminating virus. Other systems do not require infection with helper virus to recover AAV—helper functions (i.e., adenovirus E1, E2a, VA, and E4 or herpesvirus UL5, UL8, UL52, and UL29, and herpesvirus polymerase) are also supplied, in trans, by a given system. In such systems, helper functions can be supplied by transient transfection of cells with constructs that encode helper functions, or cells can be engineered to stably contain genes encoding helper functions, expression of which can be controlled at a transcriptional and/or posttranscriptional level.

In yet another embodiment, the present disclosure provides a system in which transgene flanked by ITRs and rep/cap genes are introduced into insect host cells by infection with insect virus(e.g., baculovirus)-based constructs. Such production systems are known in the art (see generally, e.g., Zhang et al., 2009, Human Gene Therapy 20:922-929). Methods of making and using these and other AAV production systems are also described in U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065, each of which is incorporated in its entirety herein by reference.

b. Methods of Treatment

In some embodiments, technologies of the present disclosure are used to treat subjects with or at risk of hearing loss. For example, in some embodiments, a subject has an autosomal dominant hearing loss attributed to at least one variant of a KCNQ4 gene. It will be understood by those in the art that many different changes (e.g., substitutions, deletions, additions, etc.) in a KCNQ4 gene that can result in or risk causing hearing loss. In some such embodiments, one or more changes in a KCNQ4 sequence results in a loss-of-function KCNQ4 gene variant.

In some embodiments, a subject experience hearing loss can be evaluated to determine if and where one or more mutations may exists that may cause hearing loss. In some such embodiments, status of a KCNQ4 gene product (e.g., polynucleotide, e.g., polypeptide) or function can be evaluated (e.g., via protein or sequencing analyses). In some embodiments of any methods described herein, a subject may be a mammal. In some embodiments, a mammal is a rodent, a non-human primate, or a human. In some embodiments of any methods described herein, a subject is an adult, a teenager, a juvenile, a child, a toddler, an infant, newborn, or fetus. In some embodiments of any methods described herein, a subject is 1-5, 1-10, 1-20, 1-30, 1-40, 1-50, 1-60, 1-70, 1-80, 1-90, 1-100, 1-110, 2-5, 2-10, 10-20, 20-30, 30-40, 40-50, 50-60, 60-70, 70-80, 80-90, 90-100, 100-110, 10-30, 10-40, 10-50, 10-60, 10-70, 10-80, 10-90, 10-100, 10-110, 20-40, 20-50, 20-60, 20-70, 20-80, 20-90, 20-100, 20-110, 30-50, 30-60, 30-70, 30-80, 30-90, 30-100, 40-60, 40-70, 40-80, 40-90, 40-100, 50-70, 50-80, 50-90, 50-100, 60-80, 60-90, 60-100, 70-90, 70-100, 70-110, 80-100, 80-110, or 90-110 years of age. In some embodiments a subject is 1, 2, 3, 4, 5, 6, 7, 8, 9, 10, or 11 months of age. In some embodiments, a subject is 1, 2, 3, 4, 5 or more weeks of age. In some embodiments, a subject is between 23 and 42 gestational weeks of age (i.e., a fetus of a gestational age).

In some embodiments of any methods described herein, such methods may result in improvement in hearing (e.g., any metrics for determining improvement in hearing described herein) in a subject in need thereof for at least 10 days, at least 15 days, at least 20 days, at least 25 days, at least 30 days, at least 35 days, at least 40 days, at least 45 days, at least 50 days, at least 55 days, at least 60 days, at least 65 days, at least 70 days, at least 75 days, at least 80 days, at least 85 days, at least 100 days, at least 105 days, at least 110 days, at least 115 days, at least 120 days, at least 5 months, at least 6 months, at least 7 months, at least 8 months, at least 9 months, at least 10 months, at least 11 months, or at least 12 months.

In some embodiments a subject (e.g., a mammal, e.g., a human) has or is at risk of developing non-syndromic sensorineural hearing loss. In some embodiments a subject (e.g., a mammal, e.g., a human) has been previously identified as having a mutation in a KCNQ4 gene. In some embodiments a subject (e.g., a mammal, e.g., a human) has any mutations in a KCNQ4 gene that are described herein or are known in the art to be associated with non-symptomatic sensorineural hearing loss.

In some embodiments a subject (e.g., a mammal, e.g., a human) has been identified as being a carrier of a mutation in a KCNQ4 gene (e.g., via genetic testing). In some embodiments a subject (e.g., a mammal, e.g., a human) has been identified as having a mutation in a KCNQ4 gene and has been diagnosed with non-symptomatic sensorineural hearing loss. In some embodiments a subject (e.g., a mammal, e.g., a human) has been identified as having non-symptomatic sensorineural hearing loss.

In some embodiments, a subject (e.g., a mammal, e.g., a human) has been identified as being at risk of hearing loss (e.g., at risk of being a carrier of a gene mutation, e.g., a KCNQ4 gene mutation). In some such embodiments, a subject (e.g., a mammal, e.g., a human) may have, e.g., certain risk factors (e.g., parent or symptoms of hearing loss) of hearing loss or risk of hearing loss. In some such embodiments, a subject (e.g., a mammal, e.g., a human) has been identified as being a carrier of a mutation in a KCNQ4 gene (e.g., via genetic testing) that has not previously been identified (i.e., is not a published, known variant of a KCNQ4 gene sequence). In some such embodiments, identified mutations may be novel (i.e., not previously described in the literature), and methods of treatment for a subject suffering from or susceptible to hearing loss can be personalized to one or more mutation(s) of a particular subject (e.g., mammal, e.g., human).

In some embodiments, successful treatment of non-symptomatic sensorineural hearing loss can be determined in a subject using any conventional functional hearing tests known in the art. Non-limiting examples of functional hearing tests are various types of audiometric assays (e.g., pure-tone testing, speech testing, middle ear test, auditory brainstem response, and otoacoustic emissions).

c. Methods of Increasing Expression of KCNQ4

The present disclosure provides methods of increasing expression of a functional (e.g., gain-of-function) KNCQ4 gene product (e.g., a wild type KCNQ4, e.g., a codon optimized KCNQ4, a Kv7.4 protein) in a cell (e.g., a mammalian cell). In some such embodiments, such methods comprise introducing any compositions (e.g., a construct encoding a functional KCNQ4, e.g., a wild type KCNQ4, e.g., a codon optimized KCNQ4) described herein into a cell (e.g., a mammalian cell). In some embodiments, introduction is in vitro. In some embodiments, introduction is ex vivo. In some embodiments, introduction is in vivo.

In some such embodiments, a cell is a mammalian cell. In some embodiments, a mammalian cell is a cochlear cell. In some embodiments, a cochlear cell is a hair cell. In some embodiments, a cochlear hair cell is an inner hair cell. In some embodiments, a cochlear hair cell is an outer hair cell. In such some embodiments, a mammalian cell is a human cell (e.g., a human cochlear outer hair cell).

Methods for introducing any compositions described herein into a mammalian cell are known in the art (e.g., via lipofection or through use of a viral construct, e.g., any viral constructs described herein). In some embodiments, an increase in expression of a KCNQ4 gene product (e.g., a gene product encoding a functional KCNQ4) is determined, e.g., as compared to a control or to a level of expression of a KCNQ4 gene product prior to introduction of a composition as described herein. In some embodiments a decrease in a KCNQ4 variant gene product (e.g., a gene product that occurs as a result of a variant KCNQ4 gene) occurs concomitantly or sequentially with an increase in a functional KCNQ4 gene product.

d. Methods of Decreasing Expression of Endogenous KCNQ4 and/or Replacing Endogenous KCNQ4

The present disclosure provides methods of decreasing expression of an endogenous KNCQ4 gene product (e.g., a wild type KCNQ4, e.g., a loss-of-function KCNQ4 variant) in a cell (e.g., a mammalian cell) and/or replacing it with a different, functional (e.g., gain-of-function) KCNQ4 (e.g., a codon-optimized KCNQ4). In some embodiments, as described herein, endogenous KCNQ4 may be replaced by a codon-optimized version that has been engineered to resist inhibitory nucleic acid-mediated degradation (e.g., miRNA-mediated degradation). In some such embodiments, miRNA-mediated degradation is due to endogenous and/or exogenously introduced miRNAs. In some embodiments as described herein, a genome editing strategy may be or comprise, e.g., introduction of an exogenous miRNA and may be preceding, followed or substantially simultaneously include replacement of endogenous KCNQ4 gene with a codon-optimized KCNQ4 gene.

In some such embodiments, such methods comprise introducing any compositions (e.g., a construct encoding a functional KCNQ4, e.g., a wild type KCNQ4, e.g., a codon optimized KCNQ4) described herein into a cell (e.g., a mammalian cell). In some embodiments, introduction is in vitro. In some embodiments, introduction is ex vivo. In some embodiments, introduction is in vivo.

In some such embodiments, a cell is a mammalian cell. In some embodiments, the mammalian cell is a cochlear cell. In some embodiments, a cochlear cell is a hair cell. In some embodiments, a cochlear hair cell is an inner hair cell. In some embodiments, a cochlear hair cell is an outer hair cell. In some such embodiments, a mammalian cell is a human cell (e.g., a human cochlear outer hair cell).

Methods for introducing any compositions described herein into a mammalian cell are known in the art (e.g., via lipofection or through use of a viral construct, e.g., any viral constructs described herein). In some embodiments, a decrease in expression of an endogenous KCNQ4 gene product is determined, e.g., as compared to a control or to a level of expression of a KCNQ4 gene product prior to introduction of a composition as described herein. In some embodiments a decrease in an endogenous KCNQ4 gene product occurs concomitantly or sequentially with an increase in a codon-optimized KCNQ4 gene product.

e. Methods of Decreasing Expression of a Loss-of-Function KCNQ4 Variant

The present disclosure provides methods of decreasing expression of a loss-of-function KNCQ4 variant gene product in a cell (e.g., a mammalian cell). In some such embodiments, such methods comprise introducing any compositions (e.g., an inhibitory nucleic acid) described herein into a cell (e.g., a mammalian cell). In some embodiments, introduction is in vitro. In some embodiments, introduction is ex vivo. In some embodiments, introduction is in vivo.

In some such embodiments, a cell is a mammalian cell. In some embodiments, a mammalian cell is a cochlear cell. In some embodiments, a cochlear cell is a hair cell. In some embodiments, a cochlear hair cell is an inner hair cell. In some embodiments, a cochlear hair cell is an outer hair cell. In some such embodiments, a mammalian cell is a human cell (e.g., a human cochlear outer hair cell).

Methods for introducing any compositions described herein into a mammalian cell are known in the art (e.g., via lipofection or use of a viral construct, e.g., any viral constructs described herein). A decrease in expression of a KCNQ4 variant gene product is determined, e.g., as compared to a control or to a level of expression of aKCNQ4 variant gene product prior to introduction of a composition as described herein. In some embodiments a decrease in KCNQ4 variant occurs concomitantly or sequentially with an increase in functional KCNQ4.

### 5. Administration

Provided herein are technologies comprising, among other things, therapeutic delivery systems for treating hearing loss (e.g., nonsyndromic sensorineural hearing loss or syndromic sensorineural hearing loss). In some embodiments the present disclosure provides compositions that are part of or comprise at least one construct, e.g., viral construct, e.g., AAV construct. In some embodiments, an inhibitory nucleic acid of the present disclosure is included within or comprises at least one construct. In some such embodiments, a construct is an AAV construct. In some embodiments, an AAV construct is used to deliver an inhibitory nucleic acid to one or more cells. In some embodiments, an inhibitory nucleic acid is included within or comprises at least one construct. In some such embodiments, a construct is an AAV construct. In some embodiments, an AAV construct is used to deliver an inhibitory nucleic acid to one or more cells. In some such embodiments, a construct encodes an inhibitory nucleic acid that may, in some embodiments, decrease expression of a potassium channel gene product, e.g., a potassium channel mRNA, e.g., KCNQ4 mRNA). In some embodiments, a single construct encodes more than one inhibitory nucleic acid molecule (e.g., more than one miRNA targeting sequence, etc.). In some embodiments, a single construct encodes 2, 3, 4, 5, 6, 7, 8, 9, or 10 inhibitory nucleic acid molecules.

Non-limiting examples of siRNAs that can decrease expression of a potassium channel gene product (e.g., protein) in a cell (e.g., a human cell, e.g., an outer hair cell) are described herein. As described herein, in some embodiments, a subject has been previously identified as having a loss-of-function KCNQ4 variant (e.g., a KCNQ4 gene having a sequence variation that results in a defect (e.g., decrease) in expression and/or activity of a KCNQ4 protein encoded by a gene or in a function that causes disease (e.g., DFNA2 or another dysfunction in a potassium channel, e.g., chronic depolarization leading to death). In some embodiments, prior to an introducing or administering step as provided herein, a subject is determined to have a KCNQ4 variant gene. In some embodiments, a method of treatment comprises detecting a variation in a KCNQ4 gene in a subject. In some embodiments, a method of treating comprises identifying or diagnosing a subject as having non-symptomatic sensorineural hearing loss.

As described herein, technologies provided by the present disclosure (e.g., expression systems, inhibitory RNAs or genome editing systems, etc.) can be implemented (e.g., administered or delivered to a cell or a subject) in a variety of ways, and different implementations may be suitable for distinct applications. For example, in some embodiments, technologies of the present disclosure are used to increase a level of a gene, decrease a level of a gene, and/or increase and decrease levels of certain genes or gene variants, simultaneously. For example, in some embodiments, technologies of the present disclosure include increasing expression of a functional KCNQ4 gene product, decreasing expression of a loss-of-function KCNQ4 gene variant, replacing a functional KCNQ4 gene product with a codon-optimized version of a KCNQ4 gene product, or any combination of increasing and decreasing thereof.

a. Routes of Administration

In some embodiments, the present disclosure provides various routes of and formulations for administration. As will be known to one of skill in the art, pharmaceutical forms suitable for injectable use include sterile aqueous solutions or dispersions and sterile powders for extemporaneous preparation of sterile injectable solutions or dispersions. Dispersions may also be prepared in glycerol, liquid polyethylene glycols, and mixtures thereof and in oils.

Under ordinary conditions of storage and use, these preparations contain a preservative to prevent growth of microorganisms. In many cases the form is sterile and fluid to the extent that easy syringability exists. It must be stable under the conditions of manufacture and storage and must be preserved against contaminating action of microorganisms, such as bacteria and fungi. In some embodiments, a carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by use of a coating, such as lecithin, by maintenance of the required particle size in the case of dispersion and by use of surfactants. Prevention of action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride.

Prolonged absorption of injectable compositions can be brought about use in compositions of agents delaying absorption, for example, aluminum monostearate and gelatin. For administration of an injectable aqueous solution, for example, a solution may be suitably buffered, if necessary, and a liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 ml of isotonic NaCl solution and either added to 1000 ml of hypodermoclysis fluid or injected at a proposed site of infusion, (see for example, “Remington's Pharmaceutical Sciences” 15th Edition, pages 1035-1038 and 1570-1580, which is incorporated in its entirety herein by reference). Some variation in dosage will necessarily occur depending on condition of a host. A person responsible for administration will, in any event, determine an appropriate dose for an individual host.

In some embodiments, sterile injectable solutions are prepared by incorporating active rAAV in a required amount in an appropriate solvent with various other ingredients enumerated herein, as required, followed by filtered sterilization. Generally, dispersions are prepared by incorporating various sterilized active ingredients into a sterile vehicle which contains basic dispersion medium and required other ingredients from those enumerated above. In the case of sterile powders for preparation of sterile injectable solutions, in some embodiments, preferred methods of preparation are vacuum-drying and freeze-drying techniques which yield a powder of active ingredient plus any additional desired ingredient from a previously sterile-filtered solution thereof.

In some embodiments, rAAV compositions disclosed herein may also be formulated in a neutral or salt form. Pharmaceutically-acceptable salts, include acid addition salts (formed with free amino groups of a given protein) and which are formed with inorganic acids such as, for example, hydrochloric or phosphoric acids, or such organic acids as acetic, oxalic, tartaric, mandelic, and the like. Salts formed with free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions can be administered in a manner compatible with the dosage formulation and in such amount as is therapeutically effective. Formulations are easily administered in a variety of dosage forms such as injectable solutions, drug-release capsules, and the like.

Delivery vehicles such as liposomes, nanocapsules, microparticles, microspheres, lipid particles, vesicles, and the like, may be used for introduction of compositions of the present disclosure into suitable host cells. In particular, in some embodiments, rAAV-construct delivered transgenes may be formulated for delivery either encapsulated in a lipid particle, a liposome, a vesicle, a nanosphere, or a nanoparticle or the like.

Such formulations may be preferred for introduction of pharmaceutically acceptable formulations of nucleic acids or rAAV constructs disclosed herein. Formation and use of liposomes is generally known to those of skill in the art. Recently, liposomes were developed with improved serum stability and circulation half-times (U.S. Pat. No. 5,741,516, which is incorporated in its entirety herein by reference). Further, various methods of liposome and liposome like preparations as potential drug carriers have been described (U.S. Pat. Nos. 5,567,434; 5,552,157; 5,565,213; 5,738,868 and 5,795,587, each of which is incorporated in its entirety herein by reference).

Liposomes have been used successfully with a number of cell types that are normally resistant to transfection by other procedures. In addition, liposomes are free of DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining efficacy of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 Tm. Sonication of MLVs results in formation of small unilamellar vesicles (SUVs) with diameters in a range of approximately 200 to 500.ANG., containing an aqueous solution in the core.

Alternatively, nanocapsule formulations of rAAV may be used. Nanocapsules can generally entrap substances in a stable and reproducible way. To avoid side effects due to intracellular polymeric overloading, such ultrafine particles (sized around 0.1 Tm) should be designed using polymers able to be degraded in vivo. Biodegradable polyalkyl-cyanoacrylate nanoparticles that meet these requirements are contemplated for use.

In addition to methods of delivery described above, the following techniques are also contemplated as alternative methods of delivering the rAAV compositions to a host. Sonophoresis (i.e., ultrasound) has been used and described in U.S. Pat. No. 5,656,016, which is incorporated in its entirety herein by reference, as a device for enhancing the rate and efficacy of drug permeation into and through a circulatory system. Other drug delivery alternatives contemplated are intraosseous injection (U.S. Pat. No. 5,779,708, which is incorporated in its entirety herein by reference), microchip devices (U.S. Pat. No. 5,797,898, which is incorporated in its entirety herein by reference), ophthalmic formulations (Bourlais et al., 1998, which is incorporated in its entirety herein by reference), transdermal matrices (U.S. Pat. Nos. 5,770,219 and 5,783,208, each of which is incorporated in its entirety herein by reference) and feedback-controlled delivery (U.S. Pat. No. 5,697,899, which is incorporated in its entirety herein by reference).

In some embodiments, administration of any compositions of the present disclosure may be carried out in any convenient manner, including by aerosol inhalation, injection, ingestion, transfusion, implantation or transplantation. Compositions described herein may be administered to a patient trans arterially, subcutaneously, intradermally, intranodally, intramedullary, intramuscularly, by intravenous (i.v.) injection, or intraperitoneally. In some embodiments, a nucleic acid composition of the present disclosure is administered to a patient by intradermal or subcutaneous injection. In some embodiments, a nucleic composition of the present disclosure is administered by i.v. injection.

b. Dosing

i. rAAV-KCNQ4-Inhibitory-RNA

In some embodiments, any of the methods disclosed herein comprise a dose-escalation study to assess safety and tolerability in subjects, e.g., mammals, e.g., humans, e.g., patients, with hearing loss. In some embodiments, a composition disclosed herein, e.g., rAAV-KCNQ4-Inhibitory-RNA, is administered at a dosing regimen disclosed herein. In some embodiments, the dosing regimen comprises either unilateral or bilateral intracochlear administrations of a dose, e.g., as described herein, of a composition disclosed herein, e.g., rAAV-KCNQ4-Inhibitory-RNA. In some embodiments, the dosing regimen comprises delivery in a volume of at least 0.001 mL, 0.005 mL, 0.01 mL, at least 0.02 mL, at least 0.03 mL, at least 0.04 mL, at least 0.05 mL, at least 0.06 mL, at least 0.07 mL, at least 0.08 mL, at least 0.09 mL, at least 0.10 mL, at least 0.11 mL, at least 0.12 mL, at least 0.13 mL, at least 0.14 mL, at least 0.15 mL, at least 0.16 mL, at least 0.17 mL, at least 0.18 mL, at least 0.19 mL, or at least 0.20 mL per cochlea. In some embodiments, the dosing regimen comprises delivery in a volume of at most 0.30 mL, at most 0.25 mL, at most 0.20 mL, at most 0.15 mL, at most 0.14 mL, at most 0.13 mL, at most 0.12 mL, at most 0.11 mL, at most 0.10 mL, at most 0.09 mL, at most 0.08 mL, at most 0.07 mL, at most 0.06 mL, at most 0.05 mL, at most 0.01 mL, at most 0.005 mL, or at most 0.001 mL per cochlea. In some embodiments, the dosing regimen comprises delivery in a volume of about 0.001 mL, 0.005 mL, 0.01 mL, 0.05 mL, about 0.06 mL, about 0.07 mL, about 0.08 mL, about 0.09 mL, about 0.10 mL, about 0.11 mL, about 0.12 mL, about 0.13 mL, about 0.14 mL, or about 0.15 mL per cochlea, depending on the population. In some embodiments, the dosing regimen comprises delivery in a volume of at least 0.001 mL, 0.005 mL, 0.01 mL, at least 0.02 mL, at least 0.03 mL, at least 0.04 mL, at least 0.05 mL, at least 0.06 mL, at least 0.07 mL, at least 0.08 mL, at least 0.09 mL, at least 0.10 mL, at least 0.11 mL, at least 0.12 mL, at least 0.13 mL, at least 0.14 mL, at least 0.15 mL, at least 0.16 mL, at least 0.17 mL, at least 0.18 mL, at least 0.19 mL, or at least 0.20 mL per cochlea. In some embodiments, the dosing regimen comprises delivery in a volume of at most 0.30 mL, at most 0.25 mL, at most 0.20 mL, at most 0.15 mL, at most 0.14 mL, at most 0.13 mL, at most 0.12 mL, at most 0.11 mL, at most 0.10 mL, at most 0.09 mL, at most 0.08 mL, at most 0.07 mL, at most 0.06 mL, at most 0.05 mL, at most 0.01 mL, at most 0.005 mL, or at most 0.001 mL per cochlea. In some embodiments, the dosing regimen comprises delivery in a volume of about 0.001 mL, about 0.005 mL, about 0.01 mL, 0.05 mL, about 0.06 mL, about 0.07 mL, about 0.08 mL, about 0.09 mL, about 0.10 mL, about 0.11 mL, about 0.12 mL, about 0.13 mL, about 0.14 mL, or about 0.15 mL per cochlea, depending on the population.

In some embodiments, a method disclosed herein evaluates the safety and tolerability of escalating doses of a composition disclosed herein, e.g., rAAV-KCNQ4-Inhibitory-RNA, administered via intracochlear injection to a subject, e.g., 18 to 80 years of age, with hearing loss.

In some embodiments, a method disclosed herein evaluates the safety and tolerability of escalating doses of a composition disclosed herein, e.g., rAAV-KCNQ4-Inhibitory-RNA, administered via intraocular injection to a subject, e.g., 18 to 80 years of age, with hearing loss.

In some embodiments, any of the methods disclosed herein comprise an evaluation of the safety and tolerability of a composition disclosed herein, e.g., rAAV-KCNQ4-Inhibitory-RNA. In some embodiments, evaluation of the efficacy of a composition disclosed herein, e.g., rAAV-KCNQ4-Inhibitory-RNA to treat hearing loss, is performed in a randomized, controlled setting (using a concurrent, non-intervention observation arm).

In some embodiments, any of the methods disclosed herein comprise an evaluation of the safety and tolerability of a composition disclosed herein, e.g., rAAV-KCNQ4-Inhibitory-RNA. In some embodiments, evaluation of the efficacy of a composition disclosed herein, e.g., rAAV-KCNQ4-Inhibitory-RNA to treat vision loss, is performed in a randomized, controlled setting (using a concurrent, non-intervention observation arm).

III. rAAV-KCNQ4

In some embodiments, any of the methods disclosed herein comprise a dose-escalation study to assess safety and tolerability in subjects, e.g., mammals, e.g., humans, e.g., patients, with hearing loss. In some embodiments, a composition disclosed herein, e.g., rAAV-KCNQ4, is administered at a dosing regimen disclosed herein. In some embodiments, the dosing regimen comprises either unilateral or bilateral intracochlear administrations of a dose, e.g., as described herein, of a composition disclosed herein, e.g., rAAV-KCNQ4 In some embodiments, the dosing regimen comprises delivery in a volume of at least 0.001 mL, 0.005 mL, 0.01 mL, at least 0.02 mL, at least 0.03 mL, at least 0.04 mL, at least 0.05 mL, at least 0.06 mL, at least 0.07 mL, at least 0.08 mL, at least 0.09 mL, at least 0.10 mL, at least 0.11 mL, at least 0.12 mL, at least 0.13 mL, at least 0.14 mL, at least 0.15 mL, at least 0.16 mL, at least 0.17 mL, at least 0.18 mL, at least 0.19 mL, or at least 0.20 mL per cochlea. In some embodiments, the dosing regimen comprises delivery in a volume of at most 0.30 mL, at most 0.25 mL, at most 0.20 mL, at most 0.15 mL, at most 0.14 mL, at most 0.13 mL, at most 0.12 mL, at most 0.11 mL, at most 0.10 mL, at most 0.09 mL, at most 0.08 mL, at most 0.07 mL, at most 0.06 mL, at most 0.05 mL, at most 0.01 mL, at most 0.005 mL, or at most 0.001 mL per cochlea. In some embodiments, the dosing regimen comprises delivery in a volume of about 0.001 mL, 0.005 mL, 0.01 mL, 0.05 mL, about 0.06 mL, about 0.07 mL, about 0.08 mL, about 0.09 mL, about 0.10 mL, about 0.11 mL, about 0.12 mL, about 0.13 mL, about 0.14 mL, or about 0.15 mL per cochlea, depending on the population. In some embodiments, the dosing regimen comprises delivery in a volume of at least 0.001 mL, 0.005 mL, 0.01 mL, at least 0.02 mL, at least 0.03 mL, at least 0.04 mL, at least 0.05 mL, at least 0.06 mL, at least 0.07 mL, at least 0.08 mL, at least 0.09 mL, at least 0.10 mL, at least 0.11 mL, at least 0.12 mL, at least 0.13 mL, at least 0.14 mL, at least 0.15 mL, at least 0.16 mL, at least 0.17 mL, at least 0.18 mL, at least 0.19 mL, or at least 0.20 mL per cochlea. In some embodiments, the dosing regimen comprises delivery in a volume of at most 0.30 mL, at most 0.25 mL, at most 0.20 mL, at most 0.15 mL, at most 0.14 mL, at most 0.13 mL, at most 0.12 mL, at most 0.11 mL, at most 0.10 mL, at most 0.09 mL, at most 0.08 mL, at most 0.07 mL, at most 0.06 mL, at most 0.05 mL, at most 0.01 mL, at most 0.005 mL, or at most 0.001 mL per cochlea. In some embodiments, the dosing regimen comprises delivery in a volume of about 0.001 mL, about 0.005 mL, about 0.01 mL, 0.05 mL, about 0.06 mL, about 0.07 mL, about 0.08 mL, about 0.09 mL, about 0.10 mL, about 0.11 mL, about 0.12 mL, about 0.13 mL, about 0.14 mL, or about 0.15 mL per cochlea, depending on the population.

In some embodiments, a method disclosed herein evaluates the safety and tolerability of escalating doses of a composition disclosed herein, e.g., rAAV-KCNQ4, administered via intracochlear injection to a subject, e.g., 18 to 80 years of age, with hearing loss.

In some embodiments, a method disclosed herein evaluates the safety and tolerability of escalating doses of a composition disclosed herein, e.g., rAAV-KCNQ4, administered via intraocular injection to a subject, e.g., 18 to 80 years of age, with hearing loss.

In some embodiments, any of the methods disclosed herein comprise an evaluation of the safety and tolerability of a composition disclosed herein, e.g., rAAV-KCNQ4. In some embodiments, evaluation of the efficacy of a composition disclosed herein, e.g., rAAV-KCNQ4 to treat hearing loss, is performed in a randomized, controlled setting (using a concurrent, non-intervention observation arm).

In some embodiments, any of the methods disclosed herein comprise an evaluation of the safety and tolerability of a composition disclosed herein, e.g., rAAV-KCNQ4. In some embodiments, evaluation of the efficacy of a composition disclosed herein, e.g., rAAV-KCNQ4 to treat vision loss, is performed in a randomized, controlled setting (using a concurrent, non-intervention observation arm).

### 6. Delivery Strategies

a. Nucleic Acid-Based Delivery of Inhibitory RNAs or Genome Editing Systems

In some embodiments, a genome editing system is implemented for one or more nucleic acids encoding a CRISPR nuclease and gRNA components described herein (optionally with one or more additional components). In some embodiments, a genome editing system is implemented as one or more constructs comprising such nucleic acids, for instance a viral construct such as an adeno-associated virus; and in some embodiments, a genome editing system is implemented as a combination of any of the foregoing. Additional or modified implementations that operate according to principles set forth herein will be apparent to the skilled artisan and are within the scope of this disclosure.

In one non-limiting embodiment, a construct drives expression of a genome editing system. The art is replete with suitable constructs that are useful in the present invention. Constructs to be used are suitable for replication and, optionally, integration in eukaryotic cells. Typical constructs contain transcription and translation terminators, initiation sequences, and promoters useful for regulation of expression of a desired nucleic acid sequence. Constructs of the present invention may also be used for nucleic acid standard gene delivery protocols. Methods for gene delivery are known in the art (U.S. Pat. Nos. 5,399,346, 5,580,859 & 5,589,466, incorporated by reference herein in their entireties).

CRISPR/Cas gene disruption occurs when a guide nucleic acid sequence specific for a target gene and a Cas endonuclease are introduced into a cell and form a complex that enables a Cas endonuclease to introduce a double strand break at a target gene. In some embodiments, a CRISPR system comprises an expression construct, such as, but not limited to, an pAd5F35-CRISPR construct. In other embodiments, the Cas expression construct induces expression of Cas9 endonuclease. Other endonucleases may also be used, including but not limited to Cpf1, T7, Cas3, Cas8a, Cas8b, Cas10d, Cse1, Csy1, Csn2, Cas4, Cas10, Csm2, Cmr5, Fok1, other nucleases known in the art, and any combination thereof.

In some embodiments, inducing a Cas expression construct comprises exposing a cell to an agent that activates an inducible promoter in a Cas expression construct. In such embodiments, a Cas expression construct includes an inducible promoter, such as one that is inducible by exposure to an antibiotic (e.g., by tetracycline or a derivative of tetracycline, for example doxycycline). However, it should be appreciated that other inducible promoters can be used. An inducing agent can be a selective condition (e.g., exposure to an agent, for example an antibiotic) that results in induction of an inducible promoter. This results in expression of a Cas expression construct.

In other embodiments, one or more constructs driving expression of one or more elements of a CRISPR system are introduced into a host cell, such that expression of elements of a CRISPR system direct formation of a CRISPR complex at one or more target sites. For example, a Cas enzyme, a guide sequence linked to a tracr-mate sequence, and a tracr sequence could each be operably linked to separate regulatory elements on separate constructs. Alternatively, two or more elements expressed from the same or different regulatory elements may be combined in a single construct, with one or more additional constructs providing any components of a CRISPR system not included in a first construct. CRISPR system elements that are combined in a single construct may be arranged in any suitable orientation, such as one element located 5′ with respect to (“upstream” of) or 3′ with respect to (“downstream” of) a second element. A coding sequence of one element may be located on the same or opposite strand of a coding sequence of a second element, and oriented in the same or opposite direction. In some embodiments, a single promoter drives expression of a transcript encoding a CRISPR enzyme and one or more of a guide sequence, tracr mate sequence (optionally operably linked to a guide sequence), and a tracr sequence embedded within one or more intron sequences (e.g., each in a different intron, two or more in at least one intron, or all in a single intron).

b. Viral Delivery of Genome Modifying Systems and/or Transgenes

In some embodiments, a construct may be provided to a cell in the form of a viral construct, e.g., an AAV construct. In some embodiments, a genome editing system is implemented using one or more constructs comprising such nucleic acids, for instance a viral construct such as an adeno-associated virus; and in some embodiments, a genome editing system is implemented as a combination of any of the foregoing. In some embodiments, a transgene, e.g., a gene encoding KCNQ4 (e.g., a functional KCNQ4) is administered using a viral construct. Viral construct technology is well known in the art and is described, for example, in Sambrook et al. (4^{th} Edition, Molecular Cloning: A Laboratory Manual, Cold Spring Harbor Laboratory, New York, 2012, which is incorporated in its entirety herein by reference), and in other virology and molecular biology manuals. Viruses, which are useful as constructs include, but are not limited to, retroviruses, adenoviruses, adeno-associated viruses, herpes viruses, Sindbis virus, gammaretrovirus and lentiviruses. In general, a suitable construct contains an origin of replication functional in at least one organism, a promoter sequence, convenient restriction endonuclease sites, and one or more selectable markers (e.g., WO 01/96584; WO 01/29058; and U.S. Pat. No. 6,326,193, each of which is incorporated in its entirety herein by reference). In some embodiments, the present disclosure provides methods of delivery comprising administering one or more constructs comprising one or more components that inhibit a KCNQ4 variant gene product and/or one or more constructs comprising one or more components that express an exogenous KCNQ4 (e.g., a functional KCNQ4, e.g., a wild type KCNQ4, e.g., a codon optimized KCNQ4). Without being bound by any particular theory, in embodiments where viral delivery of components to inhibit a KCNQ4 variant and express functional KCNQ4, a functional KCNQ4 is codon-optimized to resist impact of KCNQ4 inhibitory components that may exist and/or be introduced into a cell or subject into which or whom they were delivered (e.g., inhibitory nucleic acid, e.g., miRNA).

### 7. Devices and Surgical Methods

The present disclosure provides, among other things technologies (e.g., systems, methods, devices, etc.) that may be used, in some embodiments, for treating deafness and other hearing-associated diseases, disorders and conditions. Examples of such technologies are also included in, e.g., WO2017223193 and WO2019084145, each of which is herein incorporated by reference in its entirety. In some embodiments, for example, the present disclosure provides therapeutic delivery systems for treating hearing loss (e.g., nonsyndromic sensorineural hearing loss or syndromic sensorineural hearing loss). In some such embodiments, a therapeutic delivery system may include: (i) a medical device capable of creating one or a plurality of incisions in a round window membrane of an inner ear of a subject, and (ii) an effective dose of a composition (e.g., any compositions described herein). In some embodiments, a medical device includes a plurality of micro-needles.

AAV constructs are capable of constituting a full-length auditory polypeptide messenger RNA in a target cell of an inner ear. In some embodiments, the present disclosure provides a means for performing a surgical method, a method including the steps of: administering intra-cochlearly to a human subject in need thereof an effective dose of a therapeutic composition of the present disclosure. A therapeutic composition is capable of being administered by using a medical device including: a) means for creating one or a plurality of incisions in a round window membrane, and b) an effective dose of a therapeutic composition.

The present disclosure provides, among other things, surgical methods for treatment (e.g., prevention, reversal, mitigation, attenuation) of hearing loss. In one aspect, methods include the steps of: introducing into a cochlea of a human subject a first incision at a first incision point; and administering intra-cochlearly an effective dose of a therapeutic composition (e.g., any compositions described herein) as provided herein. In one embodiment, a therapeutic composition (e.g., any compositions described herein) is administered to a subject at a first incision point. In some embodiments, a therapeutic composition is administered to a subject into or through a first incision. In one embodiment, a therapeutic composition is administered to a subject into or through a cochlea oval window membrane. In one embodiment, a therapeutic composition is administered to a subject into or through a cochlea round window membrane.

For example, in some embodiments, a therapeutic composition is administered using a medical device capable of creating a plurality of incisions in a round window membrane. In some embodiments, a medical device includes a plurality of micro-needles. In some embodiments, to medical device includes a plurality of micro-needles including a generally circular first aspect, wherein each micro-needle includes a diameter of at least about 10 microns. In some embodiments, a medical device includes a base and/or a reservoir capable of holding a therapeutic composition. In some embodiments, a medical device includes a plurality of hollow micro-needles individually including a lumen capable of transferring a therapeutic composition. In some embodiments, a medical device includes a means for generating at least a partial vacuum.

a. Method of Introduction into Cochlea

The present disclosure provides, among other things, a method of introducing into a cochlea of a mammal (e.g., a human) a therapeutically effective amount of any compositions or systems as described herein. Also provided are methods of increasing expression of a functional KCNQ4 protein (e.g., a KCNQ4 protein that can be part of a functional potassium channel, e.g., a potassium channel that does not result in chronically depolarized cells, e.g., outer hair cells) in a cell (e.g., a hair cell, e.g., an outer hair cell) in a cochlea of a mammal (e.g., a human) that include introducing into a cochlea of a subject a therapeutically effective amount of any compositions described herein.

Also provided are methods of treating non-symptomatic sensorineural hearing loss in a subject (e.g., a human) identified as having a defective (i.e., non-functional) KCNQ4 gene product. In some such embodiments, methods include administering a therapeutically effective amount of any compositions described herein into a cochlea of a subject. In some embodiments, administration may include administering one or more compositions (e.g., one composition comprising an inhibitory nucleic acid and another composition comprising a construct encoding a functional KCNQ4; e.g., one composition comprising a construct encoding a functional KCNQ4 and another composition comprising a growth factor or other agent that will maintain ear hair cell health, etc.). In some embodiments, methods of treating may further comprise administering a cochlear implant to a subject (e.g., at substantially the same time as any compositions described herein are administered to a subject).

In some embodiments a method of treating comprises administering two or more doses of any compositions described herein. In some such embodiments, compositions are introduced or administered into a cochlea of a mammal or subject. In some embodiments a method of treating comprises introducing or administering a first dose of a composition into a cochlea of a subject, assessing hearing function of a subject following introducing or administering of a first dose, and administering at least one additional dose of a composition into a cochlea of a subject if a subject is found not to have a hearing function within a normal range (e.g., as determined using any test for hearing known in the art).

In some embodiments a method of treatment comprises intra-cochlear administration. In some such embodiments of any methods described herein, compositions are administered through use of a medical device (e.g., any exemplary medical devices described herein). In some embodiments, intra-cochlear administration can be performed as described herein or known in the art. For example, in some embodiments, a composition can be administered or introduced into a cochlea using the following surgical technique: first using visualization with a 0 degree, 2.5-mm rigid endoscope, an external auditory canal is cleared and a round knife is used to sharply delineate an approximately 5-mm tympanomeatal flap. A tympanomeatal flap is then elevated and the middle ear is entered posteriorly. The chorda tympani nerve is identified and divided, and a currette is used to remove the scutal bone, exposing the round window membrane. To enhance apical distribution of the administered or introduced composition, a surgical laser may be used to make a small 2-mm fenestration in the oval window to allow for perilymph displacement during trans-round window membrane infusion of the composition. The microinfusion device is then primed and brought into the surgical field. The device is maneuvered to the round window, and the tip is seated within the bony round window overhang to allow for penetration of the membrane by the microneedle(s). The footpedal is engaged to allow for a measured, steady infusion of the composition. The device is then withdrawn and the round window and stapes foot plate are sealed with a gelfoam patch. One of skill in the art will understand that other variations or methods of intra-cochlear administration are available. In some embodiments, any such acceptable methods may be used to deliver one or more compositions and/or treat one or more subjects as described herein.

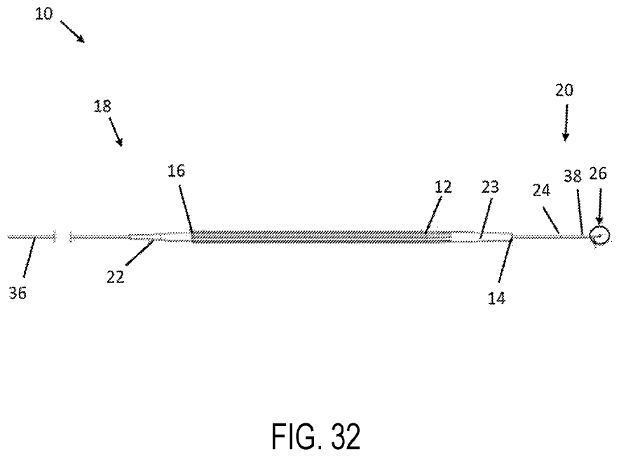

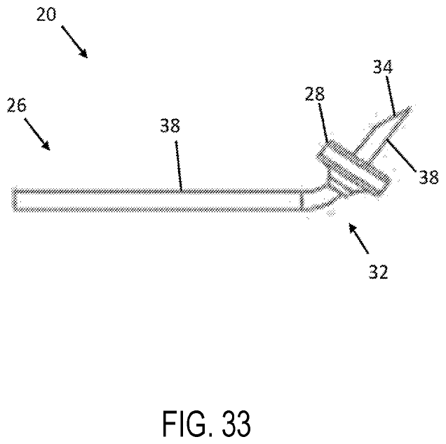

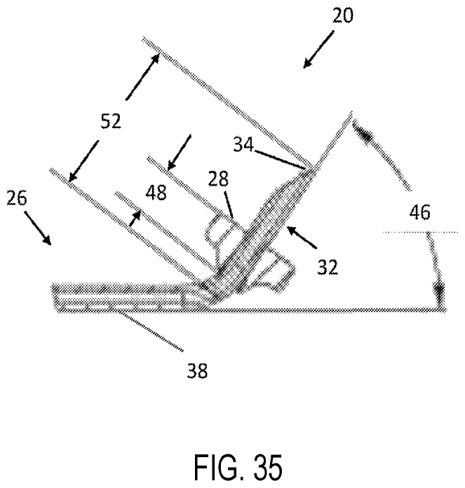

In some embodiments, an exemplary device for use in any of the methods disclosed herein is described in FIGS. 32-35. FIG. 35 illustrates an exemplary device 10 for delivering fluid to an inner ear. Device 10 includes a knurled handle 12, and a distal handle adhesive 14 (for example, an epoxy such as Loctite 4014) that couples to a telescoping hypotube needle support 24. The knurled handle 12 (or handle portion) may include kurling features and/or grooves to enhance the grip. The knurled handle 12 (or handle portion) may be from about 5 mm to about 15 mm thick or from about 5 mm to about 12 mm thick, or from about 6 mm to about 10 mm thick, or from about 6 mm to about 9 mm thick, or from about 7 mm to about 8 mm thick. The knurled handle 12 (or handle portion) may be hollow such that fluid may pass through the device 10 during use. The device 10 may also include a proximal handle adhesive 16 at a proximal end 18 of the knurled handle 12, a needle sub-assembly 26 (shown in FIG. 33) with stopper 28 (shown in FIG. 33) at a distal end 20 of the device 10, and a strain relief feature 22. Strain relief feature 22 may be composed of a Santoprene material, a Pebax material, a polyurethane material, a silicone material, a nylon material, and/or a thermoplastic elastomer. The telescoping hypotube needle support 24 surrounds and supports a bent needle 38 (shown in FIG. 33) disposed therewithin.

Referring still to FIG. 32, the stopper 28 may be composed of a thermoplastic material or plastic polymer (such as a UV-cured polymer), as well as other suitable materials, and may be used to prevent the bent needle 38 from being inserted too far into the ear canal (for example, to prevent insertion of bent needle 38 into the lateral wall or other inner ear structure). Device 10 also may include a tapered portion 23 disposed between the knurled handle 12 and the distal handle adhesive 14 that is coupled to the telescoping hypotube needle support 24. The knurled handle 12 (or handle portion) may include the tapered portion 23 at the distal end of the handle portion 12. Device 10 may also include tubing 36 fluidly connected to the proximal end 16 the device 10 and acts as a fluid inlet line connecting the device to upstream components (for example, a pump, a syringe, and/or upstream components which, in some embodiments, may be coupled to a control system and/or power supply (not shown)). In some embodiments, the bent needle 38 (shown in FIG. 33) extends from the distal end 20, through the telescoping hypotube needle support 24, through the tapered portion 23, through the knurled handle 12, and through the strain relief feature 22 and fluidly connects directly to the tubing 36. In other embodiments, the bent needle 38 fluidly connects with the hollow interior of the knurled handle (for example, via the telescoping hypotube needle support 24) which in turn fluidly connects at a proximal end 16 with tubing 36. In embodiments where the bent needle 38 does not extend all the way through the interior of the device 10, the contact area (for example, between overlapping nested hypotubes 42), the tolerances, and/or sealants between interfacing components must be sufficient to prevent therapeutic fluid from leaking out of the device 10 (which operates at a relatively low pressure (for example, from about 1 Pascal to about 50 Pa, or from about 2 Pa to about 20 Pa, or from about 3 Pa to about 10 Pa)).

FIG. 33 illustrates a sideview of the bent needle sub-assembly 26, according to aspects of the present disclosed embodiments. Bent needle sub-assembly 26 includes a needle 38 that has a bent portion 32. Bent needle sub-assembly 26 may also include a stopper 28 coupled to the bent portion 32. The bent portion 32 includes an angled tip 34 at the distal end 20 of the device 10 for piercing a membrane of the ear (for example, the RWM). The needle 38, bent portion 32, and angled top 34 are hollow such that fluid may flow therethrough. The angle 46 (as shown in FIG. 35) of the bent portion 32 may vary. A stopper 28 geometry may be cylindrical, disk-shaped, annulus-shaped, dome-shaped, and/or other suitable shapes. Stopper 28 may be molded into place onto bent portion 32. For example, stopper 28 may be positioned concentrically around the bent portion 32 using adhesives or compression fitting. Examples of adhesives include an UV cure adhesive (such as Dymax 203A-CTH-F-T), elastomer adhesives, thermoset adhesives (such as epoxy or polyurethane), or emulsion adhesives (such as polyvinyl acetate). Stopper 28 fits concentrically around the bent portion 32 such that angled tip 34 is inserted into the ear at a desired insertion depth. The bent needle 38 may be formed from a straight needle using incremental forming, as well as other suitable techniques.

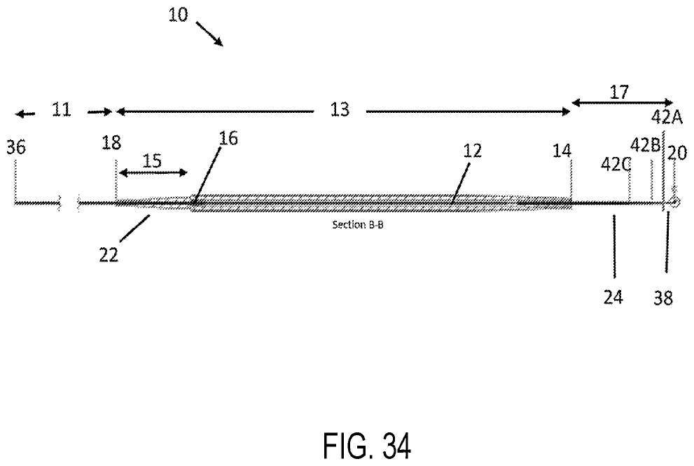

FIG. 34 illustrates a perspective view of exemplary device 10 for delivering fluid to an inner ear. Tubing 36 may be from about 1300 mm in length (dimension 11 in FIG. 34) to about 1600 mm, or from about 1400 mm to about 1500 mm, or from about 1430 mm to about 1450 mm. Strain release feature 22 may be from about 25 mm to about 30 mm in length (dimension 15 in FIG. 34), or from about 20 mm to about 35 mm in length. Handle 12 may be about 155.4 mm in length (dimension 13 in FIG. 34), or from about 150 mm to about 160 mm, or from about 140 mm to about 170 mm. The telescoping hypotube needle support 24 may have two or more nested hypotubes, for example three nested hypotubes 42A, 42B, and 42C, or four nested hypotubes 42A, 42B, 42C, and 42D. The total length of hypotubes 42A, 42B, 42C and tip assembly 26 (dimension 17 in FIG. 34) may be from about 25 mm to about 45 mm, or from about 30 mm to about 40 mm, or about 35 mm. In addition, telescoping hypotube needle support 24 may have a length of about 36 mm, or from about 25 mm to about 45 mm, or form about 30 mm to about 40 mm. The three nested hypotubes 42A, 42B, and 42C each may have a length of 3.5 mm, 8.0 mm, and 19.8 mm, respectively, plus or minus about 20%. The inner-most nested hypotube (or most narrow portion) of the telescoping hypotube needle support 24 may be concentrically disposed around needle 38.

FIG. 35 illustrates a perspective view of bent needle sub-assembly 26 coupled to the distal end 20 of device 10, according to aspects of the present disclosed embodiments. As shown in FIG. 35, bent needle sub-assembly 26 may include a needle 38 coupled to a bent portion 32. In other embodiments, the bent needle 38 may be a single needle (for example, a straight needle that is then bent such that it includes the desired angle 46). Needle 38 may be a 33-gauge needle, or may include a gauge from about 32 to about 34, or from about 31 to 35. At finer gauges, care must be taken to ensure tubing 36 is not kinked or damaged. Needle 38 may be attached to handle 12 for safe and accurate placement of needle 38 into the inner ear. As shown in FIG. 35, bent needle sub-assembly 26 may also include a stopper 28 disposed around bent portion 32. FIG. 35 also shows that bent portion 32 may include an angled tip 34 for piercing a membrane of the ear (for example, the RWM). Stopper 28 may have a height 48 of about 0.5 mm, or from about 0.4 mm to about 0.6 mm, or from about 0.3 mm to about 0.7 mm. Bent portion 32 may have a length 52 of about 1.45 mm, or from about 1.35 mm to about 1.55 mm, or from about 1.2 mm to about 1.7 mm. In other embodiments, the bent portion 32 may have a length greater than 2.0 mm such that the distance between the distal end of the stopper 28 and the distal end of the angled tip 34 is from about 0.5 mm to about 1.7 mm, or from about 0.6 mm to about 1.5 mm, or from about 0.7 mm to about 1.3 mm, or from about 0.8 mm to about 1.2 mm. FIG. 35 shows that stopper 28 may have a geometry that is cylindrical, disk-shaped, and/or dome-shaped. A person of ordinary skill will appreciate that other geometries could be used.

In some embodiments, a delivery approach as disclosed herein comprises a synthetic AAV capsid (e.g., AAV Anc80) for transduction of inner ear cells, and/or a device for targeted delivery directly to the cochlea. In certain embodiments, the present disclosure provides methods and compositions suitable for transduction of inner ear cells.

In some embodiments of any of the methods described herein, any composition described herein is administered to the subject into or through the cochlea oval window membrane. In some embodiments of any of the methods described herein, any of the compositions described herein is administered to the subject into or through the cochlea round window membrane. In some embodiments of any of the methods described herein, the composition is administered using a medical device capable of creating a plurality of incisions in the round window membrane. In some embodiments, the medical device includes a plurality of micro-needles. In some embodiments, the medical device includes a plurality of micro-needles including a generally circular first aspect, where each micro-needle has a diameter of at least about 10 microns. In some embodiments, the medical device includes a base and/or a reservoir capable of holding the composition. In some embodiments, the medical device includes a plurality of hollow micro-needles individually including a lumen capable of transferring the composition. In some embodiments, the medical device includes a means for generating at least a partial vacuum.

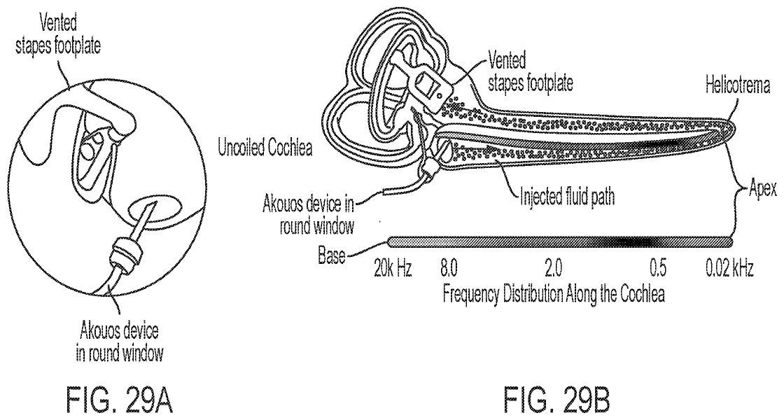

In some embodiments, also disclosed herein is a sterile, one-time use delivery device for intracochlear administration, to deliver a composition disclosed herein, e.g., rAAV-KCNQ4, e.g., rAAV-KCNQ4-Inhibitory-RNA, to perilymph fluid of inner ear through a round window membrane with a vent located in a stapes footplate. In some embodiments, in this intracochlear administration approach, a composition disclosed herein, e.g., e.g., rAAV-KCNQ4, e.g., rAAV-KCNQ4-Inhibitory-RNA, can be administered into the scala tympani through the round window membrane, with a vent in a stapes footplate within the oval window, such that composition is perfused through scala tympani, then through scala vestibuli via connection at the helicotrema, and follows the fluid path to a vent in a stapes footplate (FIGS. 29A-29B).

### 9. Evaluating Hearing Loss and Recovery

a. Auditory Testing

In some embodiments, hearing function is determined using auditory brainstem response measurements (ABRs). In some embodiments, hearing is tested by measuring distortion product optoacoustic emissions (DPOAEs). In some such embodiments, measurements are taken from one or both ears of a subject. In some such embodiments, recordings are compared to prior recordings for the same subject and/or known thresholds on such response measurements used to define, e.g., hearing loss versus acceptable hearing ranges to be defined as normal hearing. In some embodiments, a subject has ABR and/or DPOAE measurements recorded prior to receiving any treatment. In some embodiments a subject treated with one or more technologies described herein will have improvements on ABR and/or DPOAE measurements after treatment as compared to before treatment. In some embodiments, ABR and/or DPOAE measurements are taken after treatment is administered and at regular follow-up intervals post-treatment.

In some embodiments, hearing function is determined using speech pattern recognition or is determined by a speech therapist. In some embodiments, hearing function is determined by pure tone testing. In some embodiments, hearing function is determined by bone conduction testing. In some embodiments, hearing function is determined by acoustic reflex testing. In some embodiments hearing function is determined by tympanometry. In some embodiments, hearing function is determined by any combination of hearing analysis known in the art. In some such embodiments, measurements are taken holistically, and/or from one or both ears of a subject. In some such embodiments, recordings and/or professional analysis are compared to prior recordings and/or analysis for the same subject and/or known thresholds on such response measurements used to define, e.g., hearing loss versus acceptable hearing ranges to be defined as normal hearing. In some embodiments, a subject has speech pattern recognition, pure tone testing, bone conduction testing, acoustic reflex testing and/or tympanometry measurements and/or analysis conducted prior to receiving any treatment.

In some embodiments a subject treated with one or more technologies described herein will have improvements on speech pattern recognition, pure tone testing, bone conduction testing, acoustic reflex testing and/or tympanometry measurements after treatment as compared to before treatment. In some embodiments, speech pattern recognition, pure tone testing, bone conduction testing, acoustic reflex testing and/or tympanometry measurements are taken after treatment is administered and at regular follow-up intervals post-treatment.

b. Methods of Detecting or Characterizing

Methods of detecting expression and/or activity of KCNQ4 are known in the art. In some embodiments, level of expression of KCNQ4 protein can be detected directly (e.g., detecting KCNQ4 protein, detecting KCNQ4 mRNA etc.). Non-limiting examples of techniques that can be used to detect expression and/or activity of KCNQ4 directly include, e.g., real-time PCR, Western blotting, immunoprecipitation, immunohistochemistry, or immunofluorescence. In some embodiments, expression of KCNQ4 protein can be detected indirectly (e.g., through functional hearing tests, ABRs, DPOAEs, etc.).

In some embodiments, tissue samples (e.g., comprising one or more hair cells, e.g., comprising one or more hair cells) can be evaluated for morphology of hair cells before and after administration of any agents (e.g., compositions, e.g., compositions comprising constructs, etc.) as described herein. In some such embodiments, standard immunohistochemical or histological analyses can be performed. In some embodiments, if cells are used in vitro or ex vivo, additional immunocytochemical or immunhistochemical analyses can be performed. In some embodiments, one or more assays of one or more proteins or transcripts (e.g., western blot, ELISA, polymerase chain reactions) can be performed on one or more samples from a subject or an in vitro cell population.

The disclosure is further described in detail by reference to the following experimental examples. These examples are provided for purposes of illustration only, and are not intended to be limiting unless otherwise specified. Thus, the disclosure should in no way be construed as being limited to the following examples, but rather should be construed to encompass any and all variations that become evident as a result of the teaching provided herein.

For example, other assays, including those described in the Example section herein as well as those that are known in the art, can also be used in accordance with the present disclosure.

### EXAMPLES

### Example 1: shRNA-Mediated Knockdown of KCNQ4

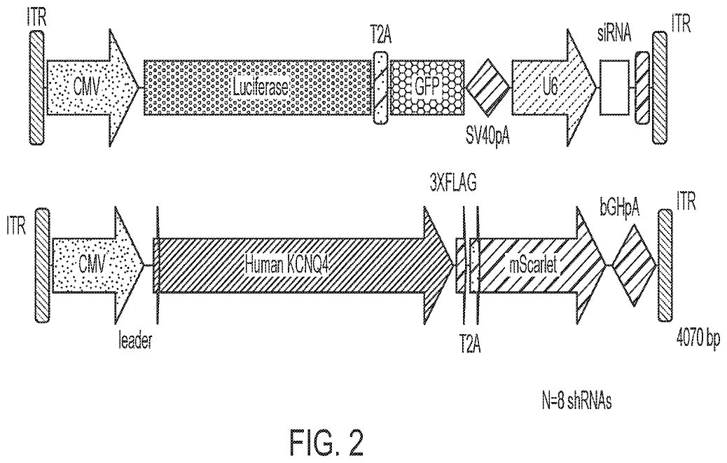

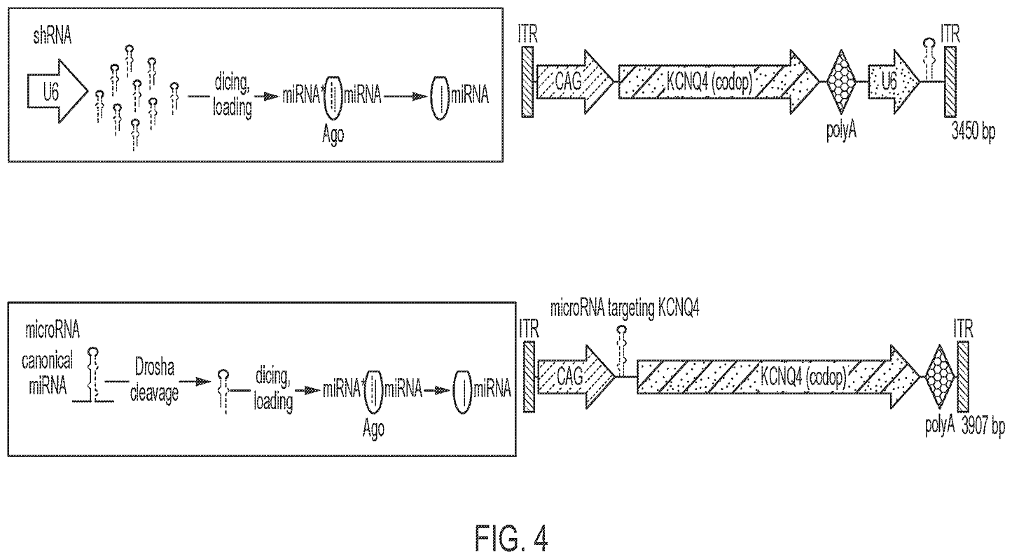

The present example demonstrates that knockdown of KCNQ4 can be achieved using shRNA and a reporter construct to measure KCNQ4 expression in human cells (FIG. 2; see also FIG. 4 (top)). FIGS.

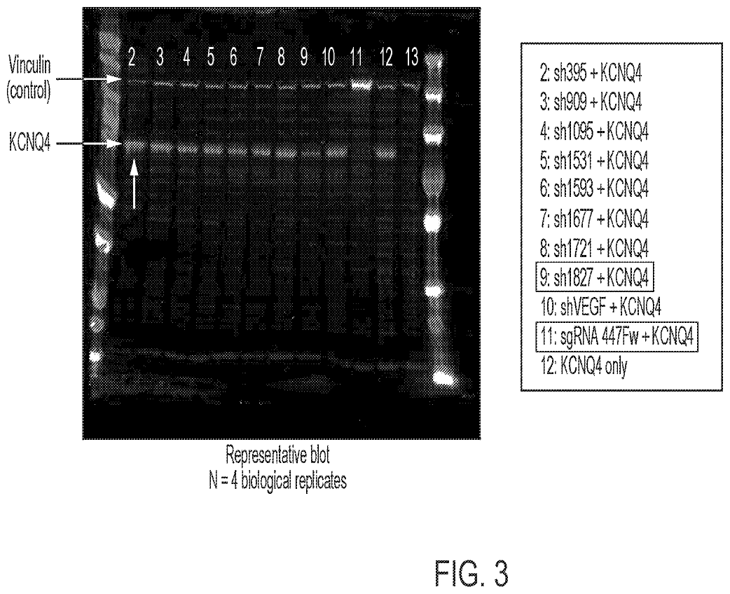

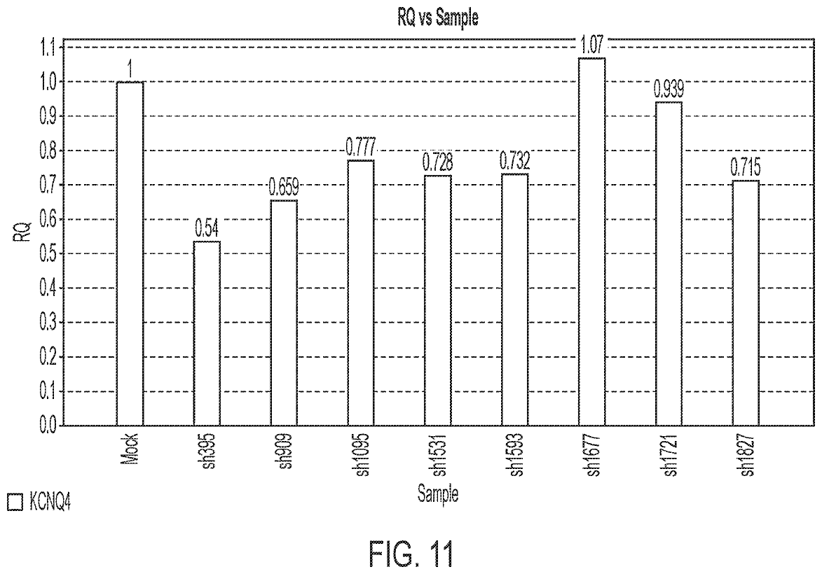

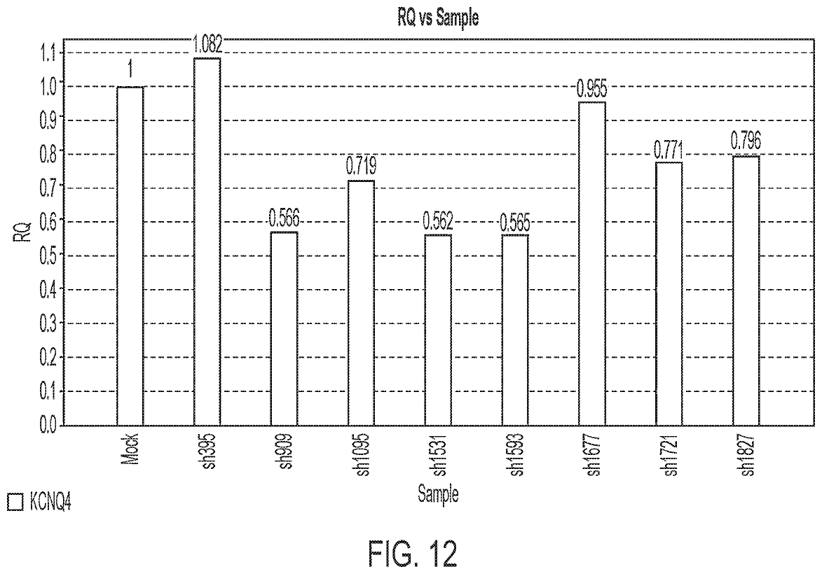

The following shRNA constructs and KCNQ4 constructs were tested in HEK293 cells: sh395, sh909, sh1095, sh1531, sh1593, sh1677, sh1721, and sh1827 (see FIG. 3). The following constructs were tested in HEK293 cells as a positive control (e.g., as a reference for maximum knockdown of KCNQ4 that can be achieved): SaCas9+sgRNA 447Fw+KCNQ4 (lane 11). A shVEGF construct (targeting VEGF) and KCNQ4 construct were tested in HEK293 cells as a negative control (lane 10). In addition, only the KCNQ4 construct was tested in HEK293 cells as a negative control (lane 12). FIG. 3 shows levels of expression of KCNQ4 in HEK293 cells with or without treatment with exemplary shRNA-mediated knockdown (lanes 2-9) and CRISPR-mediated knockdown (lane 11) (N=4 biological replicates) compared to controls (lanes 10 and 12). The level of expression of KCNQ4 was most reduced by sh1827 and sgRNA 447Fw in this example. FIGS. 11 and 12 each show efficacy of knockdown with each of the shRNA constructs as well as an empty shRNA construct (“Mock”). Levels of KCNQ4 were measured using quantitative PCR, normalized to GAPDH. Two independent biological replicates were performed (FIGS. 11 and 12), each of which had three replicates per condition.

### Example 2: miR-Mediated Knockdown of KCNQ4

The present example describes design of compositions for miR-mediated knockdown of KCNQ4. In addition, the present example demonstrates miR-mediated knockdown of KCNQ4 in human cells using compositions and methods described herein.

### Example 2.1: miRNA Construct Design and Methods

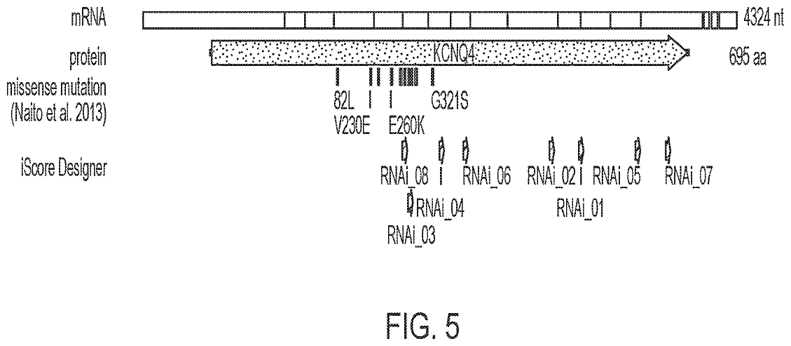

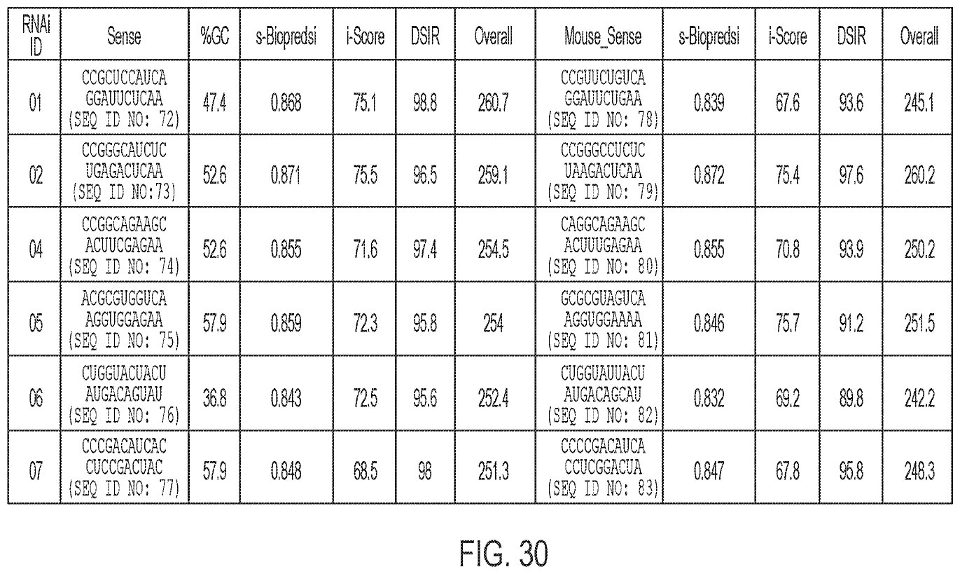

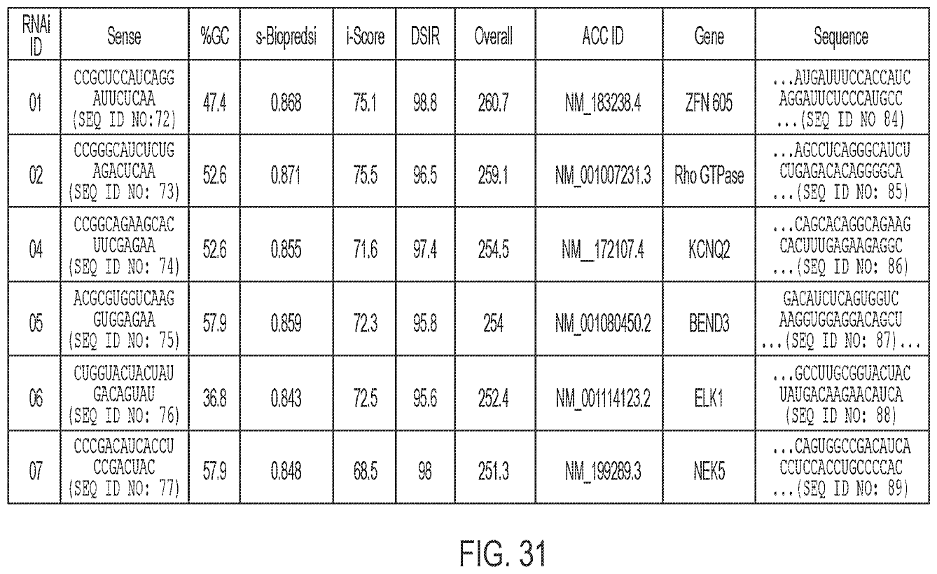

Eight miRNA targeting constructs for inhibiting KCNQ4 expression were designed using iDesigner (see FIG. 5). The top off-targets for sequence 1, 2, and 4-7 shown in FIG. 30 are displayed in FIG. 31.

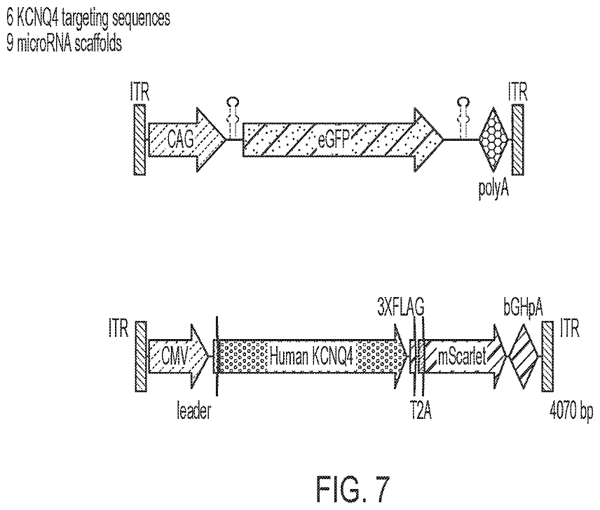

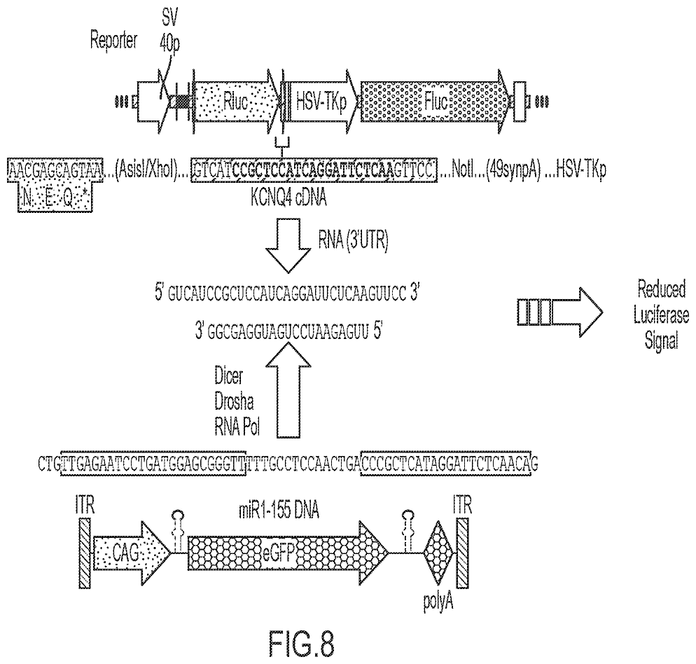

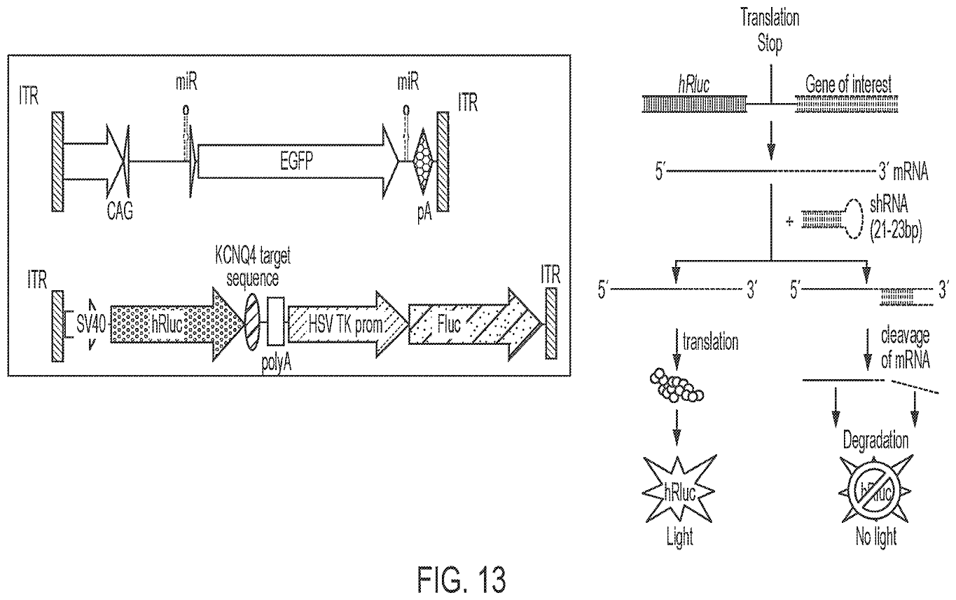

As described herein, miRNA KCNQ4 targeting sequences (based on the KCNQ4 targeting sequences presented in FIG. 30) were engineered into a variety of miRNA scaffolds, and evaluated for efficiency of KCNQ4 knockdown (see FIGS. 2, 4, 7-9). In particular, a KCNQ4-mScarlet knockdown reporter assay (FIG. 7), luciferase reporter assays (FIGS. 8 and 13), and a FLIPR assay were used to assess KCNQ4 knockdown efficiency of microRNA constructs and compositions described herein.

### Example 2.2: Results

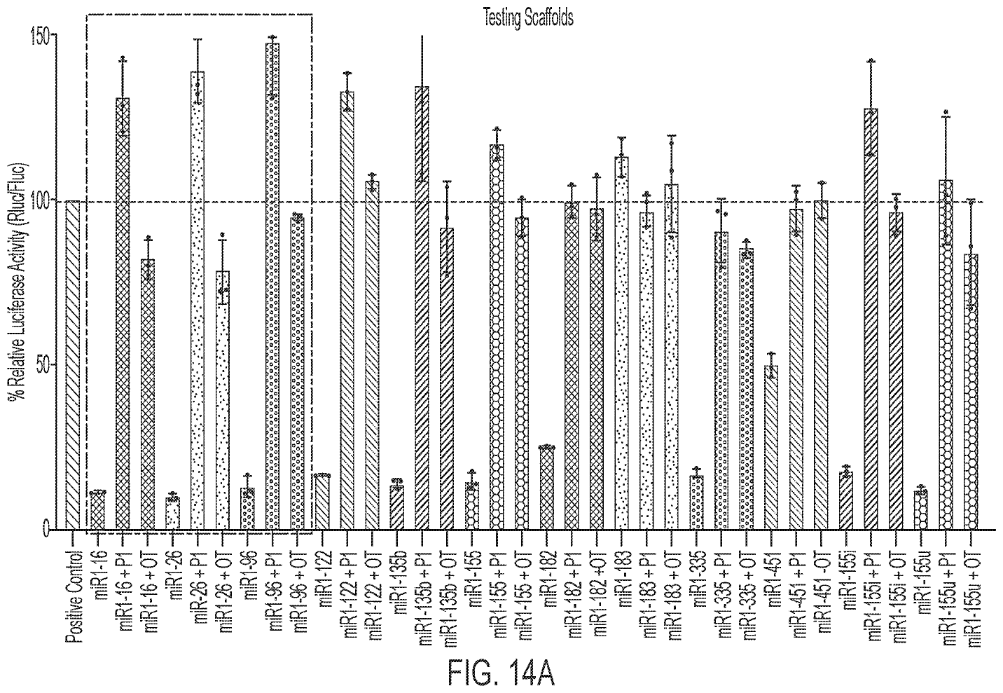

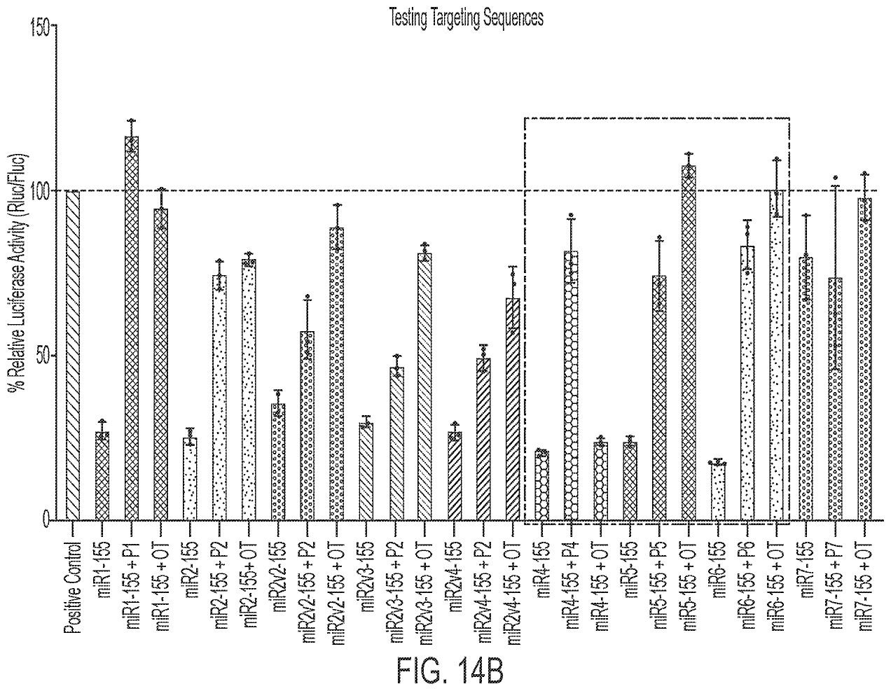

In one experiment, HEK293 cells were contacted with a control or one of fifteen different miRNA constructs targeting KCNQ4 and evaluated using a luciferase reporter assay as described herein (see FIGS. 14A-14B). In particular, six human KCNQ4 targeting sequences within a miR-155 scaffold [miR1-155 (SEQ ID NO: 56), miR2-155 (SEQ ID NO: 57), miR4-155 (SEQ ID NO: 58), miR5-155 (SEQ ID NO: 59), miR6-155 (SEQ ID NO: 60), miR7-155 (SEQ ID NO: 61)], and nine microRNA scaffolds with miR1 targeting sequences [miR1-16 (SEQ ID NO: 62), miR1-26 (SEQ ID NO: 63), miR1-96 (SEQ ID NO: 64), miR1-122 (SEQ ID NO: 65), miR1-135 (SEQ ID NO: 66), miR1-182 (SEQ ID NO: 67), miR1-183 (SEQ ID NO: 68), miR1-335 (SEQ ID NO: 69), miR1-451 (SEQ ID NO: 70)] were tested for KCNQ4 knockdown efficiency (see FIGS. 14A-14B). Immunofluorescent images were captured at 24 and 48 hours post-transfection, and cells were harvested for Dual-Glo Luciferase reporter assay as described by the manufacturer's protocol (Promega). Luminescence was measured for 10 seconds per well from the top of the plate in a standard luminometer.

Among these constructs, scaffolds exhibiting effective KCNQ4 knockdown included miR-26, miR-16, miR-96, miR-135b, and miR-155 (see FIGS. 14A-14B). The level of KCNQ4 remaining in each of these miRs was about 9.8% (miR-26); 11.4% (miR-16); 13.0% (miR-96); 14.1% (miR-135b); and 14.7% (miR-155). These data also show that, among the constructs tested, KCNQ4 targeting sequences that exhibited effective KCNQ4 knockdown were RNAi_ID6 (SEQ ID NO: 76), RNAi_ID4 (SEQ ID NO: 74), RNAi_ID5 (SEQ ID NO: 75), RNAi_ID2 (SEQ ID NO: 72), and RNAi_ID1 (SEQ ID NO: 71), respectively. The level of KCNQ4 remaining in each condition was RNAi_ID6 (17.7%); RNAi_ID4 (20.9%); RNAi_ID5 (23.7%); RNAi_ID2 (25.3%); and RNAi_ID1 (27.4%).

These results surprisingly show, for example, that miR-based constructs demonstrated substantially increased efficiency relative to the shRNA (as measured by KCNQ4 knockdown). For instance, when comparing KCNQ4 knockdown efficiency of an shRNA1095 construct (SEQ ID NO: 50) to an miR6 construct (SEQ ID NO: 211) (each of which comprise a KCNQ4 targeting sequence CTGGTACTACTATGACAGTAT), the efficiency of the miR6 construct was increased as compared to the shRNA construct (see, e.g., FIG. 3 vs FIGS. 14A-14B).

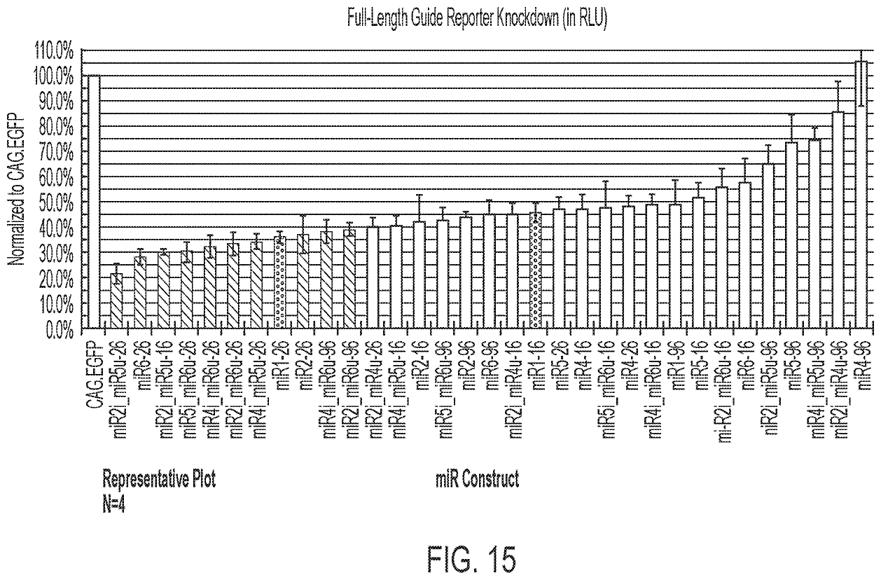

miR-mediated knockdown of KCNQ4 using a set of thirty-three miRNA compositions was also evaluated using a dual luciferase reporter assay (see TABLE 3). HEK cells (seeded at 4×10⁴ cells/well) were transfected with 600 ng of exemplary human microRNA plasmid DNA or control CAG.GFP plasmid DNA, and 400 ng of Dual Luciferase reporter DNA. Two days post-transfection, a dual luciferase assay (Promega) was run following standard protocols, and luciferase signals were read on a standard plate reader. FIG. 15 shows Renilla luciferase signals normalized to Firefly luciferase signals, and then normalized to an CAG.EGFP control plasmid (N=4; also see TABLE 3). A variety of levels of KCNQ4 knockdown with microRNA plasmids was observed, ranging from almost no knockdown to nearly 80% knockdown (see FIG. 15).

TABLE 3
Results of miR-mediated knockdown of KCNQ4 using scaffolds
and targeting sequences described herein.

| MIRNA | ID | AVERAGE | ST DEV | ST ERROR |
|---|---|---|---|---|
| CAG.EGFP | CAG | 100.0% | 0 | 0 |
| miR2i_miR5u-26** | 30 | 21.4% | 7.7% | 3.8% |
| miR6-26** | 20 | 28.0% | 6.3% | 3.1% |
| miR2i_miR5u-16** | 24 | 30.1% | 2.2% | 1.1% |
| miR5i_miR6u-26** | 34 | 30.2% | 7.7% | 3.8% |
| miR4i_miR6u-26** | 33 | 32.3% | 9.2% | 4.6% |
| miR2i_miR6u-26** | 31 | 33.4% | 9.3% | 4.6% |
| miR4i_miR5u-26** | 32 | 34.2% | 6.1% | 3.1% |
| miR1-26 | 22 | 35.9% | 4.6% | 2.3% |
| miR2-26** | 11 | 36.9% | 14.7% | 7.4% |
| miR4i_miR6u-96** | 5 | 38.3% | 8.9% | 4.5% |
| miR2i_miR6u-96 | 3 | 39.0% | 5.3% | 2.7% |
| miR2i_miR4u-26 | 29 | 39.9% | 7.5% | 3.8% |
| miR4i_miR5u-16 | 26 | 40.3% | 8.4% | 4.2% |
| miR2-16 | 10 | 42.0% | 21.7% | 10.9% |
| miR5i_miR6u-96 | 6 | 42.2% | 11.2% | 5.6% |
| miR2-96 | 12 | 43.6% | 4.9% | 2.5% |
| miR6-96 | 21 | 45.0% | 10.9% | 5.5% |
| miR2i_miR4u-16 | 23 | 45.1% | 8.7% | 4.3% |
| miR1-16 | 7 | 45.6% | 7.7% | 3.9% |
| miR5-26 | 17 | 46.7% | 9.9% | 5.0% |
| miR4-16 | 13 | 47.0% | 11.4% | 5.7% |
| miR5i_miR6u-16 | 28 | 47.6% | 21.2% | 10.6% |
| miR4-26 | 14 | 48.2% | 8.2% | 4.1% |
| miR4i_miR6u-16 | 27 | 48.3% | 9.6% | 4.8% |
| miR1-96 | 8 | 48.6% | 19.9% | 10.0% |
| miR5-16 | 16 | 51.4% | 12.4% | 6.2% |
| miR2i_miR6u-16 | 25 | 55.8% | 14.8% | 7.4% |
| miR6-16 | 19 | 57.4% | 19.2% | 9.6% |
| miR2i_miR5u-96 | 2 | 65.1% | 14.4% | 7.2% |
| miR5-96 | 18 | 73.5% | 22.3% | 11.1% |
| miR4i_miR5u-96 | 4 | 74.4% | 9.9% | 5.0% |
| miR2i_miR4u-96 | 1 | 85.5% | 24.2% | 12.1% |
| miR4-96 | 15 | 105.3% | 42.0% | 17.3% |

**MicroRNA plasmids that exhibited residual KCNQ4 reporter activity ranging from 21.4-38.3%.

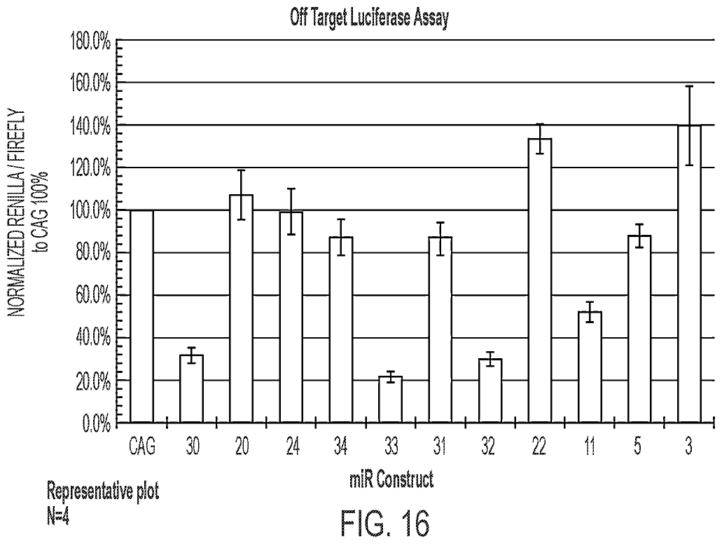

An off-target reporter assay was also used to evaluate miRNA-mediated KCNQ4 knockdown using constructs and compositions described herein (see FIG. 16). HEK cells (seeded at 4×10⁴ cells/well) were transfected with 600 ng of human microRNA plasmid DNA or control CAG.GFP plasmid DNA, and 400 ng of Dual Luciferase off-target reporter DNA. Eleven microRNA plasmids were tested (see FIG. 16, where the number on the x-axis corresponds to the number in the “ID” column of TABLE 3). Two days post-transfection, a dual luciferase assay (Promega) was run following standard protocols, and luciferase signals were read on a standard plate reader. FIG. 16 shows Renilla luciferase signals normalized to Firefly luciferase signals, and then normalized to an CAG.EGFP control construct for eleven miR constructs (N=4). As can be seen in FIG. 16, some miR constructs give more knockdown of the off-target reporter, which suggests more likelihood of off-target effects and led to exclusion of those plasmids. Constructs that showed greater than 75% signal (less than 25% knockdown) were considered for other experiments described herein.

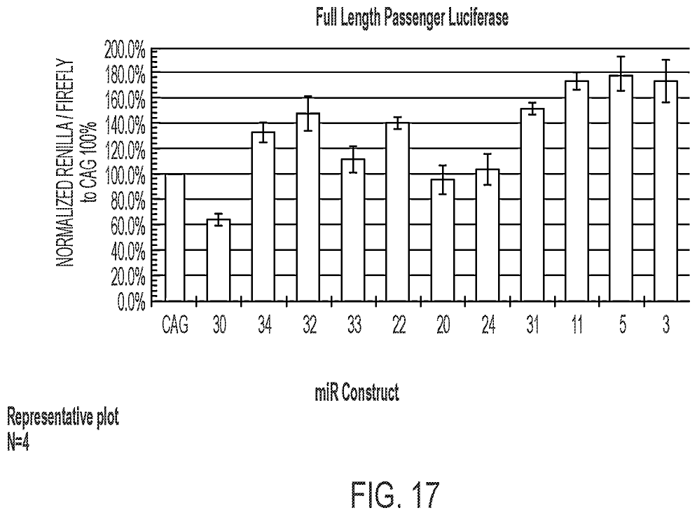

A passenger reporter assay was also used to assess miRNA-mediated KCNQ4 knockdown using constructs and compositions described herein (see FIG. 17). HEK cells (seeded at 4×10⁴ cells/well) were transfected with 600 ng of human microRNA plasmid DNA or control CAG.GFP plasmid DNA, and 400 ng of Dual Luciferase passenger reporter DNA. Eleven microRNA plasmids were tested (see FIG. 17, where the number on the x-axis corresponds to the number in the “ID” column of TABLE 3). Two days post-transfection, a dual luciferase assay (Promega) was run following standard protocols, and luciferase signals were read on a standard plate reader. FIG. 17 shows Renilla luciferase signals normalized to Firefly luciferase signals, and then normalized to a CAG.EGFP control construct. As shown in FIG. 17, some miRNA constructs result in more knockdown of passenger reporter, which suggests that passenger strand is more frequently generated from those microRNA constructs. Constructs that showed greater than 75% signal (less than 25% knockdown) were considered for other experiments described herein.

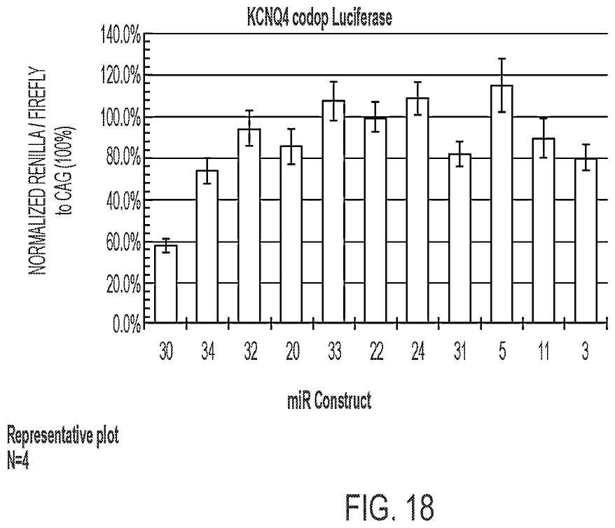

A codon-optimized KCNQ4 reporter assay was also used to evaluate miRNA-mediated KCNQ4 knockdown using constructs and compositions described herein (see FIG. 18). HEK cells (seeded at 4×10⁴ cells/well) were transfected with 600 ng of human microRNA plasmid DNA or control CAG.GFP plasmid DNA, and 400 ng of Dual Luciferase KCNQ4 codon-modified reporter DNA. Eleven microRNA plasmids were tested (see FIG. 18, where the number on the x-axis corresponds to the number in the “ID” column of TABLE 3). Two days post-transfection, a dual luciferase assay (Promega) was run following standard protocols, and luciferase signals were read on a standard plate reader. FIG. 18 shows Renilla luciferase signals normalized to Firefly luciferase signals, and then normalized to a CAG.EGFP control plasmid (N=4). Some miR constructs were observed to give more knockdown of the KCNQ4 codon-modified reporter. miR constructs that showed greater than 75% signal (less than 25% knockdown) were considered for other experiments described herein.

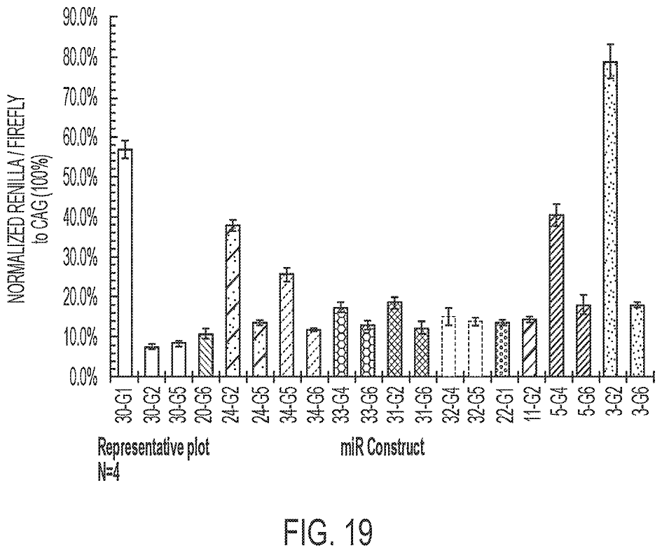

A codon-optimized KCNQ4 30-mer guide reporter assay was also used to evaluate miRNA-mediated KCNQ4 knockdown using constructs and compositions described herein (see FIG. 19). HEK cells (seeded at 4×10⁴ cells/well) were transfected with 600 ng of human microRNA plasmid DNA or control CAG.GFP plasmid DNA, and 400 ng of Dual Luciferase KCNQ4 30-mer reporter DNA. Eleven microRNA plasmids were tested (see FIG. 19, where the number on the x-axis corresponds to the number in the “ID” column of TABLE 3). Two days post-transfection, a dual luciferase assay (Promega) was run following standard protocols, and luciferase signals were read on a standard plate reader. FIG. 19 shows Renilla luciferase signals normalized to Firefly luciferase signals, and then normalized to a CAG.EGFP control plasmid (N=4). As many plasmids have two different microRNAs, each construct was tested against its specific 30-mer reporters to assess the efficacy of knockdown of each microRNA in a given plasmid. Surprisingly, some constructs show one microRNA that is more effective at knockdown than the other microRNA in that same plasmid.

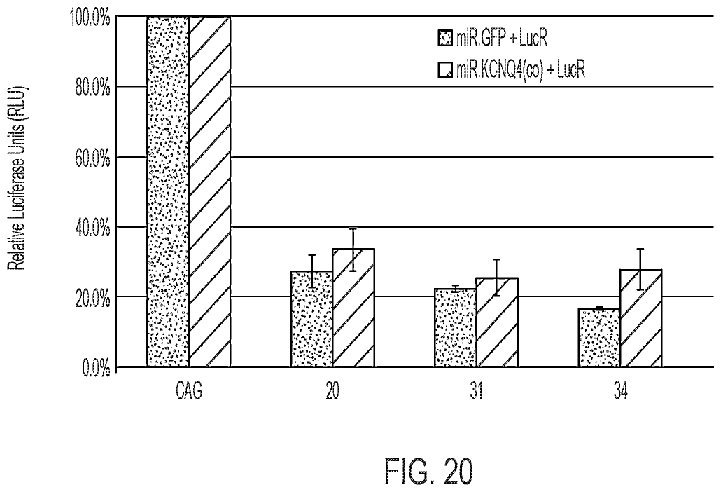

A codon-optimized KCNQ4 reporter assay was also used to evaluate miRNA-mediated KCNQ4 knockdown using constructs and compositions described herein (see FIG. 20). HEK cells (seeded at 4×10⁴ cells/well) were transfected with 600 ng of mouse microRNA plasmid DNA or control CAG.GFP plasmid DNA, and 400 ng of Dual Luciferase reporter DNA. Three microRNA plasmids were tested (see FIG. 20, where the number on the x-axis corresponds to the number in the “ID” column of TABLE 3). Two days post-transfection, a dual luciferase assay (Promega) was run following standard protocols, and luciferase signals were read on a standard plate reader. FIG. 20 shows Renilla luciferase signals normalized to Firefly luciferase signals, and then normalized to an CAG.EGFP control construct (N=3). Similar knockdown levels between microRNA constructs containing GFP and containing codon-modified KCNQ4 were observed.

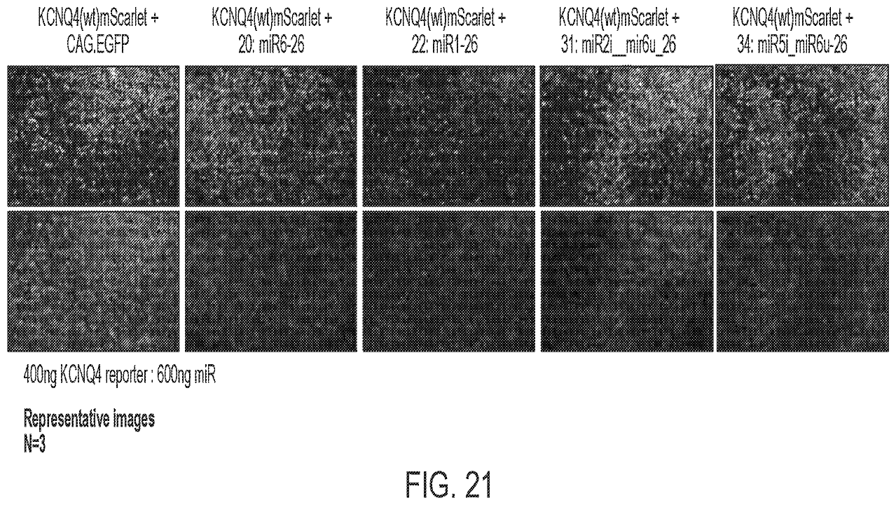

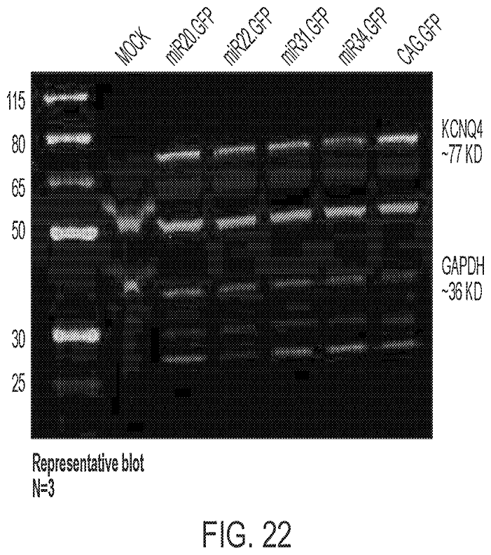

In vitro knockdown of KCNQ4 by miRNA constructs and compositions comprising human KCNQ4 targeting sequences was also evaluated in HEK cells (see FIGS. 21 and 22). HEK cells (seeded at 1.5×10⁵ cells/well) were transfected with 600 ng of human microRNA plasmid DNA or control CAG.GFP plasmid DNA, and 400 ng of human wild-type KCNQ4-mScarlet reporter DNA. Four microRNA constructs were tested (see FIG. 21, N=3). Three days post-transfection, cells were imaged for fluorescence (see FIG. 21, N=3) and then harvested for western blot analysis (FIG. 22, N=3). Reduced KCNQ4-mScarlet signals were observed with the different microRNA constructs compared to the CAG.GFP control construct. Without wishing to be bound to any theory, the present example suggests that miR constructs described herein resulted in microRNA-mediated knockdown of KCNQ4.

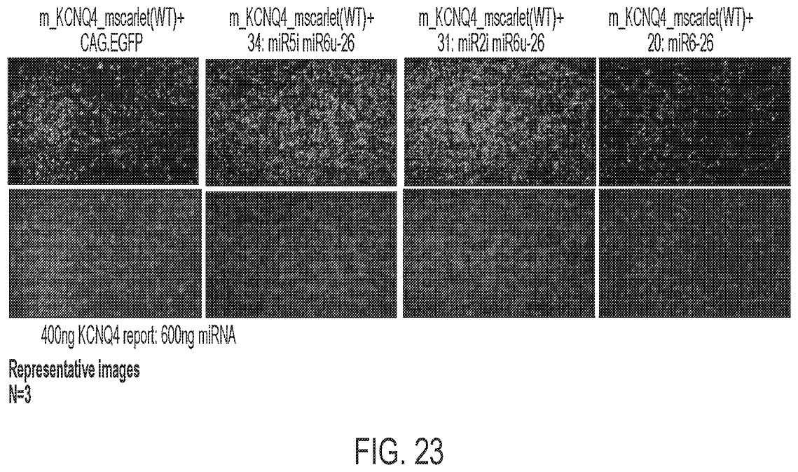

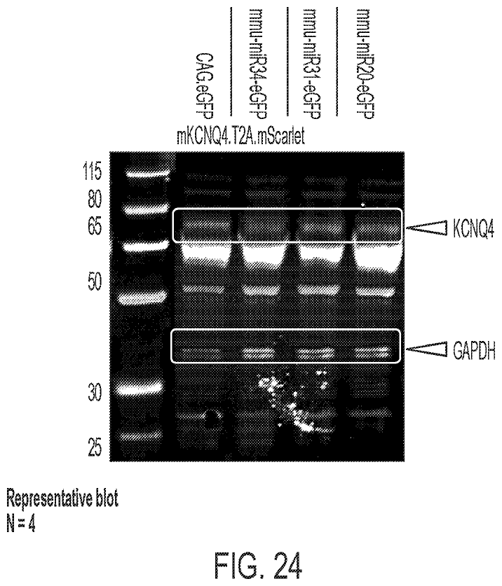

In vitro knockdown of KCNQ4 by miRNA constructs and compositions comprising mouse KCNQ4 targeting sequences were also evaluated in HEK cells (see FIGS. 23 and 24). HEK cells (seeded at 1.5×10⁵ cells/well) were transfected with 600 ng of mouse microRNA plasmid DNA or control CAG.GFP plasmid DNA, and 400 ng of mouse KCNQ4-mScarlet reporter DNA. Three microRNA constructs were tested (see FIG. 23, N=3). Three days post-transfection, cells were imaged for fluorescence (FIG. 23, N=3) and then harvested for western blot analysis (FIG. 24, N=3). Reduced KCNQ4-mScarlet signals were observed with the different microRNA constructs compared to the CAG.GFP control construct. Without wishing to be bound to any theory, the present example suggests that miR constructs described herein resulted in microRNA-mediated knockdown of KCNQ4.

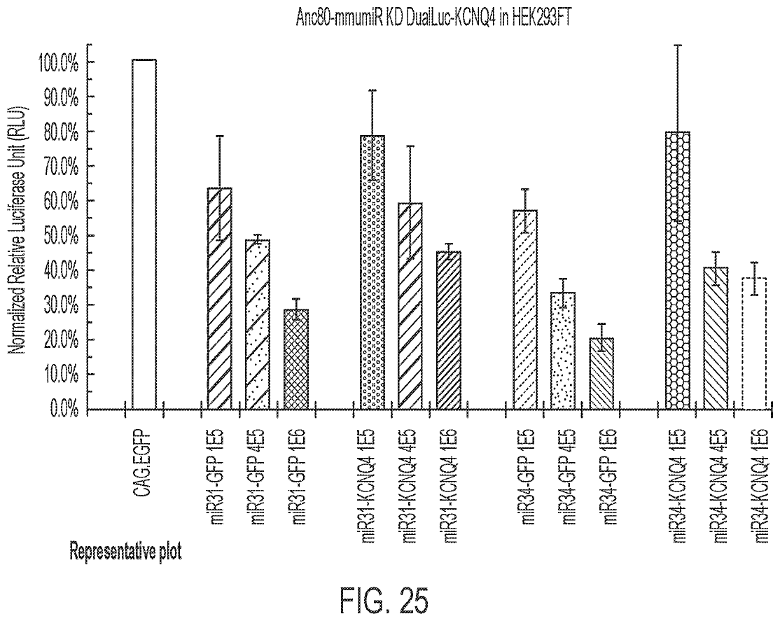

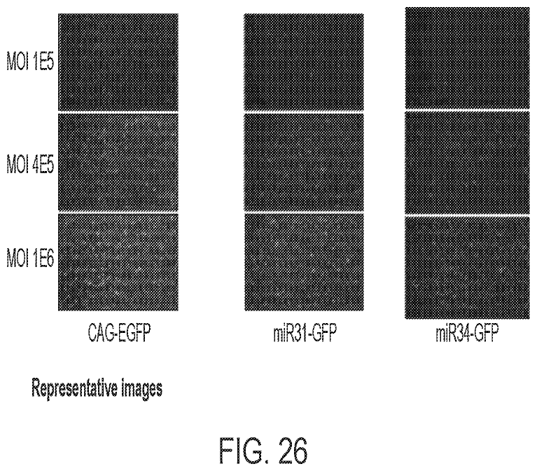

AAVAnc80-mmumiR KCNQ4 knockdown constructs and compositions comprising mouse KCNQ4 targeting sequences were also evaluated using a KCNQ4 reporter assay (see FIGS. 25 and 26). HEK cells (4×10⁴ cells/well) transduced with AAVAnc80-mmumiR knockdown vectors at three MOIs (1E5 vg/cell, 4E5 vg/cell, and 1E6 vg/cell) or AAVAnc80-CAG.EGFP control vector at three MOIs (1E5 vg/cell, 4E5 vg/cell, and 1E6 vg/cell), then transfected with 400 ng of luciferase reporter plasmid. A dual luciferase assay (Promega) was performed 72 h after transduction via standard protocols. FIG. 25 shows Renilla luciferase signals normalized to Firefly luciferase signals, then normalized to AAVAnc80-CAG.EGFP vector signal. Fluorescence images of cells before dual luciferase assay are shown below their respective histograms (see FIG. 26). A dose-dependent knockdown of dual luciferase KCNQ4 reporter with increasing vector MOI across all vectors was observed.

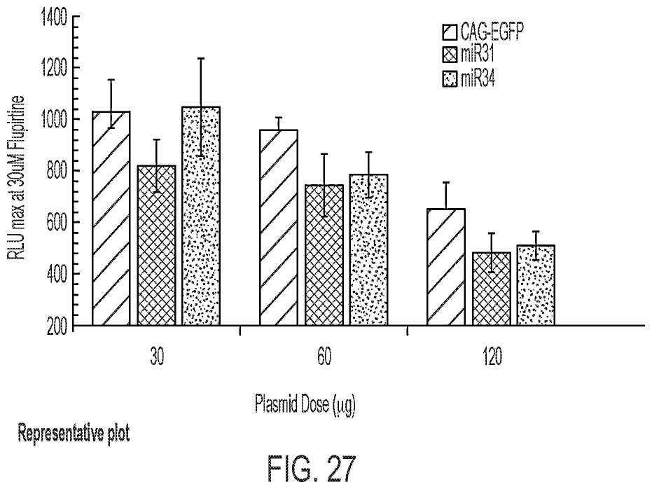

Effects on KCNQ4 channel conductance using miRNA constructs and compositions described herein were also evaluated (see FIG. 27). CHO cells stably expressing human KCNQ4 were electroporated with different amounts of human microRNA plasmid DNA (plasmid dose was in μg of DNA). An FLIPR assay was performed by adding flupirtine (KCNQ4 agonist) at none concentrations −0.01, 0.03, 0.1, 0.3, 1, 3, 10, 30, and 100 μM, N=4 wells per concentration. Fluorescence emission was measured via a thallium flux assay (potassium ion surrogate) after flupirtine addition. FIG. 27 shows maximum Relative Light Units (RLU) of different treatment conditions. A dose-dependent knockdown effect with increasing plasmid dose of the microRNA plasmids was observed, indicated by lower RLU from activated KCNQ4 in microRNA-treated cells compared to CAG.GFP treated cells.

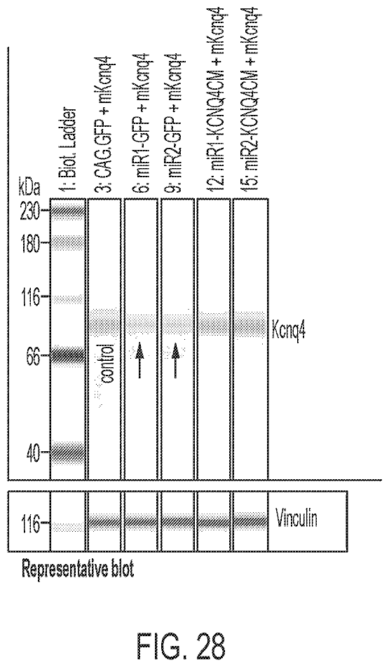

In vitro knockdown of mouse KCNQ4 in HEK cells using mouse AAVAnc80-mmumiR-GFP constructs and compositions described herein was also evaluated (see FIG. 28). HEK cells (1.5×10⁵ cells/well) were transfected with mouse KCNQ4-mScarlet reporter plasmid (“CM” refers to an codon-optimized KCNQ4 sequence), then 24 h later transduced with mouse AAVAnc80-mmumiR-GFP vectors and harvested for Western protein analysis 72 h post-transduction. Expression of codon-modified KCNQ4 protein from the AAVAnc80-mmumiR-KCNQ4CM vectors (lanes 12 and 15) were observed. Importantly, knockdown of mouse KCNQ4 levels after treatment with AAVAnc80-mmumiR-GFP vectors was observed (e.g., comparing lane 3 (control) with lanes 6 and 9).

### Example 3: Generation of a Stable Human Cell Line Expressing WT KCNQ4 and a Loss-of-Function KCNQ4 Variant

The present example seeks to overcome the problem that HEK293 cells express KCNQ4 at near-undetectable levels when assayed by western blot analysis or quantitative PCR. To address this issue, the present example describes generating a stable human cell line that expresses wild-type KCNQ4 and a loss-of-function KCNQ4 variant. A stable human cell line can be produced by knocking-in (e.g., using CRISPR technology, e.g., obtaining a cell from a patient with a loss-of-function mutation, etc.) wild-type KCNQ4 and a loss-of-function KCNQ4 variant, each with a different detectable (e.g., visualizable) reporter system, such as a reporter system provided by the present disclosure. The wild-type KCNQ4 is either a native, wild-type sequence or a codon-optimized sequence (to resist miRNA-mediated degradation). The engineered human cell line is useful for a variety of purposes, such as, e.g., screening human plasmids and viruses for effective KCNQ4 knockdown, such as those described in the present disclosure. In addition, the engineered human cell line is useful for measuring K+ currents before and after treatment in accordance with the present disclosure.

### Example 4: Knockdown of KCNQ4 in Mice

The present example describes a KCNQ4 knockdown strategy to improve hearing in mice. Wild-type (+/+), heterozygous (Dn/+), and homozygous (Dn/Dn) groups of mice will each be tested in one of four conditions Theas shown in TABLE 4, and as follows: (1) administering +/+ and Dn/+ mice eGFP and miRNA constructs described herein (Group 1); (2) administering +/+ mice eGFP only (transduction), and Dn/+ and Dn/Dn mice KCNQ and miRNA constructs described herein; (3) administering Dn/+ and Dn/Dn mice KCNQ construct only (for augmented KCNQ expression); and (4) not administering any constructs to mice (negative control). Dn transgenic (hetero and homozygous) mice have been previously described (see Kharkovets et al., Mice with altered KCNQ4+ channels implicate sensory outer hair cells in human progressive deafness, EMBO, 2006; which is incorporated in its entirety herein by reference).

As one of skill in the art will appreciate, the “Kharkovets et al.,” mice are on a C57BL/6 background, which background harbors a (naturally occurring, spontaneous) point mutation in the Cdh23 gene, which results in age-related hearing loss. The present Example describes studies to be performed in both C57BL/6-based Dn transgenic mice and mice backcrossed onto another background (e.g., FVB), that does not harbor the Cdh23 mutation, such that experiments in older mice would not be confounded by any Cdh23-mediated hearing loss. The miRNA can be delivered using AAV-based constructs comprising AAV2 ITRs and viral particles can be encapsidated with a capsid comprising Anc80.

TABLE 4
KCNQ4 knockdown strategy

| Genotype | Group 1 | Group 2 | Group 3 | Group 4 |
|---|---|---|---|---|
| +/+ | eGFP + | eGFP only | | |
| | microRNA | (transduction) | | |
| Dn/+ | eGFP + | KCNQ4 + | KCNQ4 only | Uninjected |
| | microRNA | microRNA | (augment) | |
| Dn/Dn | | KCNQ4 + | KCNQ4 only | Uninjected |
| | | microRNA | (augment) | |

In this study, miRNA or control constructs are injected into mice at a particular time point. Outer hair cell (OHC) and histological analyses may be performed 2-4 weeks post injection. Auditory brainstem response (ABR) measurements may be measured beginning at two weeks post-injection and continuing at intervals of between 6-12 weeks. miRNA or control constructs are administered to weanling or juvenile (e.g., P21, P30, P36, etc.) mice or adult (P42, P60, etc.) mice. Here, mice are injected at P21 or P30. A first auditory readout is performed at approximately 3 weeks post injection, using outer hair cell (OHC) recordings and histological analyses. Auditory brainstem response measurements are performed at 12 weeks, 24 weeks, 30 weeks, and 42 weeks of age in the mice injected at P30, and at P50, P112, and P160 days of age in the mice injected at P21.

### Example 5: Replacement and Knockdown of KCNQ4 in HEK Cells

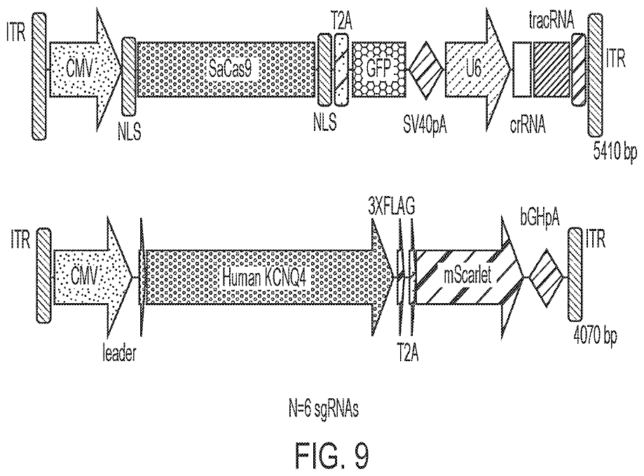

The present example describes a targeting strategy that knocks out both KCNQ4 alleles and replaces wild-type KCNQ4 with a codon-optimized KCNQ4 using a SaCas9/gRNA strategy FIG. (FIG. 9). The present example also describes an in vitro analysis of KCNQ4 knockdown in HEK cells via Western blot and quantitative PCR.

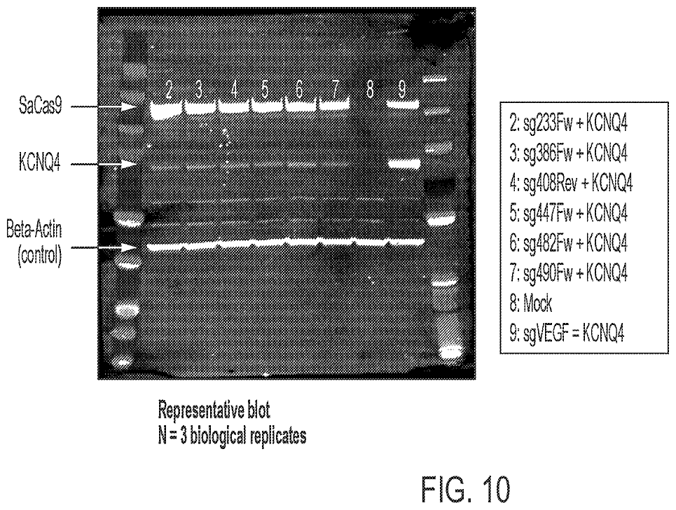

FIG. 10 shows a western blot demonstrating that transduction of SaCas9/gRNA in HEK cells significantly reduced KCNQ4. The following guides were transfected with SaCas9 and KCNQ4 DNA to determine knockdown efficiency: sg233Fw (lane 2), sg386Fw (lane 3), sg408Rev (lane 4), sg447Fw(lane 5), sg482Fw (lane 6), and sg490Fw (lane 7). An untransfected control (lane 8), sgVEGF (targeting VEGF) (lane 9), and Beta-Actin expression (lanes 2-9) were used as controls. Based on TIDE, western blot, and immunofluorescence analyses (data not shown), sgRNA 386Fw and sgRNA 408Rev inhibited KNCQ4 expression more than other sgRNAs that were tested. To expand and/or improve upon results obtained thus far, similar experiments can be performed in 3T3 cells using deadCas9 and mouse sgRNA sequences.

The present example also describes delivery of CRISPR/Cas9 using AAV (AAV-CRISPR). To begin, several constructs can be designed and characterized, in vitro, using TIDE, quantitative and western blot analyses on dual transfected cells to determine extent of DNA and RNA knockdown. The following exemplary constructs can be used: CMV.dCas9; CMV.SaCas9; Hsa KCNQ4codop.U6-386Fw (n=3 in HEK); Hsa KCNQ4codop.U6-408Rev (n=3 in HEK); Mmu KCNQ4codop.U6-386Fw (n=1, then explant); Mmu KCNQ4codop.U6-408Rev (n=1, then explant); Hsa eGFP.U6-386Fw (n=3 in HEK); Hsa eGFP.U6-408Rev (n=3 in HEK); Mmu eGFP.U6-386Fw (n=1, then explant); Mmu eGFP.U6-408Rev (n=1, then explant). Separately, a dCas9.U6 gRNA (human) is designed and characterized, and analyzed using the same methods.

An Anc80-based construct can be used for sgRNA constructs. The AAV-CRISPR construct efficiency is tested in wild-type mouse cochlear explants, human cells, or HEK cells using an eGFP virus to determine knockdown of endogenous KCNQ4 without replacement. Knockdown of KCNQ4 is evaluated using various assays such as, e.g., IHC, quantitative, and/or western blot analyses. AAV2 capsids can also be tested.

The present example also describes mouse AAV-CRISPR experiments (N=24). At 21 and 30 days post-delivery of AAV-CRISPR, mice are sacrificed and analyses (e.g., IHC analyses) for deadCas9, Myo7a (in both inner and outer hair cells) and KCNQ4 can be performed

### Example 6: Treatment of KCNQ4—Mediated Hearing Loss

The present example describes a targeting strategy that knocks out both KCNQ4 alleles to eliminate any KCNQ4 variants and simultaneously or sequentially administers a composition encoding a codon-optimized KCNQ4. This knockdown is achieved using an miRNA or SaCas9/gRNA strategy. The present example also uses, either as a pre-treatment or quality control assay, an in vitro assay of KCNQ4 knockdown in HEK cells via Western blot and quantitative PCR to determine efficacy of approach.

The compositions are administered to a subject with at least one identified loss-of-function KCNQ4 variant gene. The genomic KCNQ4 genes in the subject are suppressed and expression of the administered, codon-optimized wild type KCNQ4 is achieved. Prior to and post-administration, hearing tests may be performed (e.g., ABRs, DPOAEs).

An Anc80-based or AAV2-based particle can be used to deliver all constructs (e.g., miRNA, CRISPR/Cas9, exogenous codon-optimized KCNQ4).

### Example 7: Device Description for Suitable Delivery of Compositions to the Inner Ear

This example relates to a device suitable for the delivery of rAAV particles to the inner ear. A composition comprising rAAV particles is delivered to the cochlea of a subject using a specialized microcatheter designed for consistent and safe penetration of the round window membrane (RWM). The microcatheter is shaped such that the surgeon performing the delivery procedure can enter the middle ear cavity via the external auditory canal and contact the end of the microcatheter with the RWM. The distal end of the microcatheter may include at least one microneedle with a diameter from about 10 microns to about 1,000 microns, which produces perforations in the RWM that are sufficient to allow rAAV particles as described (e.g., comprising an rAAV construct of the present disclosure) to enter the cochlear perilymph of the scala tympani at a rate which does not damage the inner ear (e.g., a physiologically acceptable rate, e.g., a rate of approximately 30 μL/min to approximately 90 μL/min),) but small enough to heal without surgical repair. The remaining portion of the microcatheter, proximal to the microneedle(s), is loaded with the AAV particles/artificial perilymph formulation at at a defined titer (e.g., approximately 1×10¹² to 5×10¹³ vg/mL).) The proximal end of the microcatheter is connected to a micromanipulator that allows for precise, low volume infusions of approximately 30 μL to approximately 100 μL.

### Example 8: Human Clinical Example

A patient is diagnosed as having a loss-of-function KCNQ4 gene variant. Inhibitory nucleic acids are designed to target the loss-of-function KCNQ4 gene variant. The patient is put under general anesthesia. The surgeon approaches the tympanic membrane from external auditory canal, makes a small incision at the inferior edge of the external auditory canal where it meets the tympani membrane, and lifts the tympanic membrane as a flap to expose the middle ear space. A surgical laser is used to make a small opening (approximately 2 mm) in the stapes footplate. The surgeon then penetrates the round window membrane with a microcatheter loaded with a solution of a mixture of AAV-based constructs each comprising inhibitory nucleic acids to KCNQ4 or exogenous, codon-optimized KCNQ4, prepared in artificial perilymph at a titer of 1e13 vg/mL. The microcatheter is connected to a micromanipulator that infuses approximately 20 uL of the mixture at a rate of approximately 1 uL/min. At the conclusion of the infusion, the surgeon withdraws the microcatheter and patches the holes in the stapes foot plate and RWM with a gel foam patch. The procedure concludes with replacement of the tympanic membrane flap before the patient is allowed to withdraw and recover from the anesthesia.

### Example 9: Non-Invasive Prenatal Testing of Maternal Blood to Detect a KCNQ4 Mutation

Maternal blood samples (20-40 mL) are collected into Cell-free DNA tubes. At least 7 mL of plasma is isolated from each sample via a double centrifugation protocol of 2,000 g for 20 minutes, followed by 3,220 g for 30 minutes, with supernatant transfer following the first spin. cfDNA is isolated from 7-20 mL plasma using a QIAGEN QIAmp Circulating Nuclei Acid kit and eluted in 45 μL TE buffer. Pure maternal genomic DNA is isolated from the buffy coat obtained following the first centrifugation.

By combining thermodynamic modeling of the assays to select probes with minimized likelihood of probe-probe interaction with amplification approaches described previously (Stiller et al. 2009 Genome Res 19(10):1843-1848, which is incorporated herein in its entirety), multiplexing of 11,000 assays can be achieved. Maternal cfDNA and maternal genomic DNA samples are pre-amplified for 15 cycles using 11,000 target-specific assays and an aliquot is transferred to a second PCR reaction of 15 cycles using nested primers. Samples are prepared for sequencing by adding barcoded tags in a third 12-cycle round of PCR. The targets include SNPs corresponding to the greater than 30 mutations in KCNQ4 known to lead to KCNQ4 loss-of-function and/or sequences that cover all exons of KCNQ4, in order to detect any presently unknown but potentially pathogenic (e.g., loss-of-function) variant. The amplicons are then sequenced using an Illumina HiSeq sequencer. Genome sequence alignment is performed using commercially available software.

### EMBODIMENTS

Embodiment 1. A construct comprising a coding sequence operably linked to a promoter, wherein the coding sequence encodes a Kv7.4 protein.

Embodiment 2. The construct of embodiment 1, wherein the coding sequence is a KCNQ4 gene.

Embodiment 3. The construct of embodiment 2, wherein the KCNQ4 gene is a primate KCNQ4 gene.

Embodiment 4. The construct of embodiment 2 or 3, wherein the KCNQ4 gene is a human KCNQ4 gene.

Embodiment 5. The construct of embodiment 4, wherein the human KCNQ4 gene comprises a nucleic acid sequence according to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 25, SEQ ID NO: 26, SEQ ID NO: 27, SEQ ID NO: 28, SEQ ID NO: 29, SEQ ID NO: 30 or SEQ ID NO: 90.

Embodiment 6. The construct of embodiment 4 or 5, wherein the human KCNQ4 gene comprises a nucleic acid sequence according to SEQ ID NO: 9 or 10.

Embodiment 7. The construct of embodiment 1, wherein the Kv7.4 protein is a primate Kv7.4 protein.

Embodiment 8. The construct of embodiment 1 or 7, wherein the Kv7.4 protein is a human Kv7.4 protein.

Embodiment 9. The construct of embodiment 8, wherein the Kv7.4 protein comprises an amino acid sequence according to SEQ ID NO: 11, SEQ ID NO: 12, or SEQ ID NO: 13.

Embodiment 10. The construct of any one of claims 1-9, wherein the promoter is an inducible promoter, a constitutive promoter, or a tissue-specific promoter.

Embodiment 11. The construct of any one of embodiments 1-10, wherein the promotor is a cochlear hair cell-specific promoter.

Embodiment 12. The construct of embodiment 11, wherein the cochlear hair cell-specific promoter is a ATOH1 promoter, a POU4F3 promoter, a LHX3 promoter, a MYO7A promoter, a MYO6 promoter, a α9ACHR promoter, or a α10ACHR promoter.

Embodiment 13. The construct of any one of embodiments 1-10, wherein the promoter is a CAG promoter, a CBA promoter, an smCBA promoter, a CMV promoter, or a CB7 promoter.

Embodiment 14. The construct of embodiment 13, wherein the promoter comprises a nucleic acid sequence according to SEQ ID NO: 22, SEQ ID NO: 23, SEQ ID NO: 24, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308, SEQ ID NO: 309, and/or SEQ ID NO: 310.

Embodiment 15. The construct of any one of embodiments 1-14, further comprising two AAV inverted terminal repeats (ITRs), wherein the two AAV ITRs flank the coding sequence and promoter.

Embodiment 16. The construct of embodiment 15, wherein the two AAV ITRs are or are derived from AAV2 ITRs.

Embodiment 17. The construct of embodiment 15, wherein the two AAV ITRs comprise: (i) a 5′ ITR comprising a nucleic acid sequence according to SEQ ID NO: 15 and a 3′ ITR comprising a nucleic acid sequence according to SEQ ID NO: 16; or (ii) a 5′ ITR comprising a nucleic acid sequence according to SEQ ID NO: 19 and a 3′ ITR comprising a nucleic acid sequence according to SEQ ID NO: 20.

Embodiment 18. The construct of embodiment 1, wherein the construct comprises a nucleic acid sequence according to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 25, SEQ ID NO: 26, SEQ ID NO: 27, SEQ ID NO: 28, SEQ ID NO: 29, SEQ ID NO: 30, SEQ ID NO: 90, or SEQ ID NO: 91.

Embodiment 19. The construct of embodiment 1, wherein the construct comprises a nucleic acid sequence according to one or more of SEQ ID NOs: 1-41 and/or 42-70 and/or 96-97.

Embodiment 20. A construct comprising a coding sequence operably linked to a promoter, wherein the coding sequence encodes a KCNQ4 inhibitory nucleic acid, which comprises a nucleotide sequence complementary to a KCNQ4 gene.

Embodiment 21. The construct of embodiment 20, wherein the KCNQ4 inhibitory nucleic acid is an miRNA, an siRNA, or shRNA.

Embodiment 22. The construct of embodiment 20 or 21, wherein the KCNQ4 inhibitory RNA comprises a sequence according to SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO:63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 96, or SEQ ID NO: 97.

Embodiment 23. The construct of embodiment 20, wherein the KCNQ4 inhibitory RNA is a gRNA.

Embodiment 24. The construct of embodiment 20 or 23, wherein the KCNQ4 inhibitory RNA comprises a sequence according to SEQ ID NO: 42, SEQ ID NO: 43, SEQ ID NO: 44, SEQ ID NO: 45, SEQ ID NO: 46, SEQ ID NO: 47, SEQ ID NO: 48.

Embodiment 25. The construct of embodiment 20, wherein the KCNQ4 gene is a primate KCNQ4 gene.

Embodiment 26. The construct of embodiment 20 or 25, wherein the KCNQ4 gene is a human KCNQ4 gene.

Embodiment 27. The construct of embodiment 26, wherein the human KCNQ4 gene comprises a nucleic acid sequence according to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 25, SEQ ID NO: 26, SEQ ID NO: 27, SEQ ID NO: 28, SEQ ID NO: 29, SEQ ID NO: 30 or SEQ ID NO: 90.

Embodiment 28. The construct of any one of claims 20-27, wherein the promoter is an inducible promoter, a constitutive promoter, or a tissue-specific promoter.

Embodiment 29. The construct of any one of embodiments 20-28, wherein the promotor is a cochlear hair cell-specific promoter.

Embodiment 30. The construct of embodiment 29, wherein the cochlear hair cell-specific promoter is a ATOH1 promoter, a POU4F3 promoter, a LHX3 promoter, a MYO7A promoter, a MYO6 promoter, a α9ACHR promoter, or a α10ACHR promoter.

Embodiment 31. The construct of any one of embodiments 20-28, wherein the promoter is an H1 or U6 promoter.

Embodiment 32. The construct of embodiment 31, wherein the promoter comprises a nucleic acid sequence according to SEQ ID NO: 22, SEQ ID NO: 23, SEQ ID NO: 24, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308, SEQ ID NO: 309, and/or SEQ ID NO: 310.

Embodiment 33. The construct of any one of embodiments 20-32, further comprising two AAV inverted terminal repeats (ITRs), wherein the two AAV ITRs flank the coding sequence and promoter.

Embodiment 34. The construct of embodiment 33, wherein the two AAV ITRs are or are derived from AAV2 ITRs.

Embodiment 35. The construct of embodiment 33, wherein the two AAV ITRs comprise: (i) a 5′ ITR comprising a nucleic acid sequence according to SEQ ID NO: 15 and a 3′ ITR comprising a nucleic acid sequence according to SEQ ID NO: 16; or (ii) a 5′ ITR comprising a nucleic acid sequence according to SEQ ID NO: 19 and a 3′ ITR comprising a nucleic acid sequence according to SEQ ID NO: 20.

Embodiment 36. The construct of embodiment 1, wherein the construct comprises a nucleic acid sequence according to any of SEQ ID NOs: 1-10.

Embodiment 37. The construct of embodiment 1, wherein the construct comprises a nucleic acid sequence according to any of SEQ ID NOs: 25-30 or 90-91.

Embodiment 38. An AAV particle comprising the construct of any one of embodiments 1-19.

Embodiment 39. An AAV particle comprising the construct of any one of embodiments 20-37.

Embodiment 40. The AAV particle of embodiment 38 or 39, further comprising an AAV capsid, wherein the AAV capsid is or is derived from an AAV2, AAV3, AAV4, AAV5, AAV6, AAV7, AAV8, AAV9, AAV10, AAV-rh8, AAV-rh10, AAV-rh39, AAV-rh43 or AAV Anc80 capsid.

Embodiment 41. The AAV particle of embodiment 40, wherein the AAV capsid is an AAV Anc80 capsid.

Embodiment 42. A composition comprising: (i) the construct of any one of embodiments 1-19; (ii) the construct of any one of embodiments 20-37; or (iii) a combination thereof.

Embodiment 43. A composition comprising the AAV particle of any one of embodiments 38-41.

Embodiment 44. A composition comprising: (i) the AAV particle of embodiment 38; (ii) the AAV particle of embodiment 39; or (iii) a combination thereof.

Embodiment 45. The composition of embodiment 44, wherein the AAV particle of (i), (ii), or both further comprise an AAV capsid, wherein the AAV capsid is or is derived from an AAV2, AAV3, AAV4, AAV5, AAV6, AAV7, AAV8, AAV9, AAV10, AAV-rh8, AAV-rh10, AAV-rh39, AAV-rh43 or AAV Anc80 capsid.

Embodiment 46. The composition of embodiment 44, wherein the AAV capsid of the AAV particle of (i), (ii), or both is an AAV Anc80 capsid.

Embodiment 47. The composition of any one of embodiments 42-46, wherein the composition is a pharmaceutical composition.

Embodiment 48. The composition of embodiment 47, further comprising a pharmaceutically acceptable carrier.

Embodiment 49. A cell comprising the composition of any one of embodiments 42-48.

Embodiment 50. The cell of embodiment 49, wherein the cell is in vivo, ex vivo, or in vitro.

Embodiment 51. The cell of embodiment 49 or 50, wherein the cell is a mammalian cell.

Embodiment 52. The cell of embodiment 51, wherein the mammalian cell is a human cell.

Embodiment 53. The cell of embodiment 52, wherein the cell is immortalized to generate a stable cell line.

Embodiment 54. The cell of embodiment 52, wherein the human cell is in the ear of a subject.

Embodiment 55. The cell of embodiment 52, wherein at least one copy of an endogenous KCNQ4 gene has at least one sequence variation.

Embodiment 56. The cell of embodiment 55, wherein the at least one sequence variation results in a loss-of-function gene product.

Embodiment 57. A system comprising the composition of any one of embodiments 42-48.

Embodiment 58. A method comprising contacting an inner ear cell with the composition of any one of embodiments 42-48.

Embodiment 59. The method of embodiment 58, where the inner ear cell is an outer hair cell.

Embodiment 60. The method of embodiment 58 or 59, wherein the inner ear cell is in the ear of a subject.

Embodiment 61. The method of embodiment 58 or 59, wherein the inner ear cell is in vitro or ex vivo.

Embodiment 62. A method comprising, contacting a cell with: (i) the construct of any one of embodiments 1-38; and (ii) one or more plasmids comprising an AAV Rep gene, AAV Cap gene, AAV VA gene, AAV E2a gene, and AAV E4 gene.

Embodiment 63. The method of embodiment 62, where the cell is an inner ear cell.

Embodiment 64. The method of embodiment 63, wherein the inner ear cell is outer hair cell.

Embodiment 65. The method of embodiment 63, wherein the inner ear cell is in the ear of a subject.

Embodiment 66. The method of embodiment 58 or 59, wherein the inner ear cell is in vitro or ex vivo.

Embodiment 67. A method comprising introducing the composition of any one of embodiments 42-48 into the inner ear of a subject.

Embodiment 68. The method of embodiment 67, wherein the composition is introduced into the cochlea of the subject.

Embodiment 69. The method of embodiment 67 or 68, wherein the composition is introduced via a round window membrane injection.

Embodiment 70. The method of any one of embodiments 60, 65, or 67-69, further comprising measuring a hearing level of the subject.

Embodiment 71. The method of embodiment 70, a hearing level is measured by performing an auditory brainstem response (ABR) test.

Embodiment 72. The method of embodiment 70 or 71, further comprising comparing the hearing level of the subject to a reference hearing level.

Embodiment 73. The method of embodiment 72, wherein the reference hearing level is a published or historical reference hearing level.

Embodiment 74. The method of embodiment 73, wherein the hearing level of the subject is measured after the construct of any one of embodiments 1-19 is introduced, and the reference hearing level is a hearing level of the subject that was measured before the construct of any one of embodiments 1-19 was introduced.

Embodiment 75. The method of any one of embodiments 58-74, further comprising measuring a level of a KCNQ4 gene product in a subject.

Embodiment 76. The method of embodiment 75, wherein the level of the KCNQ4 gene product is measured in the inner ear of the subject.

Embodiment 77. The method of embodiment 75, wherein the level of the KCNQ4 gene product is measured in the cochlea of the subject.

Embodiment 78. The method of any one of embodiments 58-77, further comprising comparing the level of a KCNQ4 gene product in the subject to a reference KCNQ4 gene product level.

Embodiment 79. The method of embodiment 78, wherein the reference hearing level is a published or historical reference KCNQ4 gene product level.

Embodiment 80. The method of embodiment 75, wherein the level of a KCNQ4 gene product in the subject is measured after the construct of any one of embodiments 1-37 is introduced, and the reference KCNQ4 gene product level is a KCNQ4 gene product level of the subject that was measured before the composition of any one of embodiments 1-37 was introduced.

Embodiment 81. A method of treating hearing loss comprising administering the composition of any one of embodiments 42-48 to a subject in need thereof.

Embodiment 82. A method of treating hearing loss comprising administering a particle of any one of embodiments 38-41 to a subject in need thereof.

Embodiment 83. A construct of any one of embodiments 1-37 for use in the treatment of hearing loss.

Embodiment 84. A composition of any one of embodiments 42-48 for use in the treatment of hearing loss.

Embodiment 85. A particle of any one of embodiments 38-41 for use in the treatment of hearing loss.

Embodiment 86. Use of a construct of any one of embodiments 1-37 for the manufacture of a medicament to treat hearing loss.

Embodiment 87. Use of a composition of any one of embodiments 42-48 for the manufacture of a medicament to treat hearing loss.

Embodiment 88. Use of a particle of any one of embodiments 38-41 for the manufacture of a medicament to treat hearing loss.

Embodiment 89. A cell comprising a coding sequence operably linked to a promoter, wherein the coding sequence encodes a Kv7.4 protein.

Embodiment 90. A cell comprising a coding sequence operably linked to a promoter, wherein the coding sequence encodes a loss-of-function KCNQ4 variant gene product.

Embodiment 91. A population of cells comprising one or more cells according to embodiment 89, wherein the population is or comprises a stable cell line.

Embodiment 92. The method of any one of embodiments 75-80, wherein the KCNQ4 gene product is a Kv7.4 protein.

Embodiment 93. A construct comprising an inhibitory nucleic acid, wherein the inhibitory nucleic acid is or comprises one or more of miR1-155; miR2-155; miR4-155; miR5-155; miR6-155; miR7-155 miR1-16; miR1-26; miR1-96; miR1-122; miR1-135; miR1-182; miR1-183; miR1-335; miR1-451.

Embodiment 94. An miRNA selected from the group consisting of miR1-155; miR2-155; miR4-155; miR5-155; miR6-155; miR7-155; miR1-16; miR1-26; miR1-96; miR1-122; miR1-135; miR1-182; miR1-183; miR1-335; miR1-451 and combinations thereof.

Embodiment 95. A kit comprising a composition of any one of embodiments 1-94.

Embodiment 96. The kit of embodiment 95, wherein the composition is pre-loaded in a device.

Embodiment 97. The kit of embodiment 96, wherein the device is a microcatheter.

Embodiment 98. The kit of embodiment 97, wherein the microcatheter is shaped such that it can enter the middle ear cavity via the external auditory canal and contact the end of the microcatheter with the RWM.

Embodiment 99. The kit of embodiment 97 or 98, wherein a distal end of the microcatheter is comprised of at least one microneedle with diameter of between 10 and 1,000 microns.

Embodiment 100. The kit of any one of embodiments 95-99, further comprising a device.

Embodiment 101. The kit of embodiment 100, wherein the device is a device described in FIGS. 32-35 or a device as described herein.

Embodiment 102. The kit of embodiment 101, wherein the device comprises a needle comprising a bent portion and an angled tip.

### EQUIVALENTS

It is to be understood that the words which have been used are words of description rather than limitation, and that changes may be made within the purview of the appended claims without departing from the true scope and spirit of the invention in its broader aspects.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

It is to be understood that while the disclosure has been described in conjunction with the detailed description thereof, the foregoing description is intended to illustrate and not limit the scope of the present disclosure, which is defined by the scope of the appended claims. Other aspects, advantages, and modifications are within the scope of the following claims.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

1. A construct comprising a coding sequence operably linked to a promoter, wherein the coding sequence encodes a Kv7.4 protein.

2. The construct of claim 1, wherein the coding sequence is a KCNQ4 gene.

3. The construct of claim 2, wherein the KCNQ4 gene is a primate KCNQ4 gene.

4. The construct of claim 2 or 3, wherein the KCNQ4 gene is a human KCNQ4 gene.

5. The construct of claim 4, wherein the human KCNQ4 gene comprises a nucleic acid sequence according to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 25, SEQ ID NO: 26, SEQ ID NO: 27, SEQ ID NO: 28, SEQ ID NO: 29, SEQ ID NO: 30 or SEQ ID NO: 90.

6. The construct of claim 4 or 5, wherein the human KCNQ4 gene comprises a nucleic acid sequence according to SEQ ID NO: 9 or 10.

7. The construct of claim 1, wherein the Kv7.4 protein is a primate Kv7.4 protein.

8. The construct of claim 1 or 7, wherein the Kv7.4 protein is a human Kv7.4 protein.

9. The construct of claim 8, wherein the Kv7.4 protein comprises an amino acid sequence according to SEQ ID NO: 11, SEQ ID NO:12, or SEQ ID NO:13.

10. The construct of any one of claims 1-9, wherein the promoter is an inducible promoter, a constitutive promoter, or a tissue-specific promoter.

11. The construct of any one of claims 1-10, wherein the promotor is a cochlear hair cell-specific promoter.

12. The construct of claim 11, wherein the cochlear hair cell-specific promoter is a ATOH1 promoter, a POU4F3 promoter, a LHX3 promoter, a MYO7A promoter, a MYO6 promoter, a α9ACHR promoter, or a α10ACHR promoter.

13. The construct of any one of claims 1-10, wherein the promoter is a CAG promoter, a CBA promoter, an smCBA promoter, a CMV promoter, or a CB7 promoter.

14. The construct of claim 13, wherein the promoter comprises a nucleic acid sequence according to SEQ ID NO: 22, SEQ ID NO: 23, SEQ ID NO: 24, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308, SEQ ID NO: 309, and/or SEQ ID NO: 310.

15. The construct of any one of claims 1-14, further comprising two AAV inverted terminal repeats (ITRs), wherein the two AAV ITRs flank the coding sequence and promoter.

16. The construct of claim 15, wherein the two AAV ITRs are or are derived from AAV2 ITRs.

17. The construct of claim 15, wherein the two AAV ITRs comprise:
(i) a 5′ ITR comprising a nucleic acid sequence according to SEQ ID NO: 15 and a 3′ ITR comprising a nucleic acid sequence according to SEQ ID NO: 16; or
(ii) a 5′ ITR comprising a nucleic acid sequence according to SEQ ID NO: 19 and a 3′ ITR comprising a nucleic acid sequence according to SEQ ID NO: 20.

18. The construct of claim 1, wherein the construct comprises a nucleic acid sequence according to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 25, SEQ ID NO: 26, SEQ ID NO: 27, SEQ ID NO: 28, SEQ ID NO: 29, SEQ ID NO: 30, SEQ ID NO: 90, or SEQ ID NO: 91.

19. The construct of claim 1, wherein the construct comprises a nucleic acid sequence according to one or more of SEQ ID NOs: 1-41 and/or 42-70 and/or 96-97.

20. A construct comprising a coding sequence operably linked to a promoter, wherein the coding sequence encodes a KCNQ4 inhibitory nucleic acid, which comprises a nucleotide sequence complementary to a KCNQ4 gene.

21. The construct of claim 20, wherein the KCNQ4 inhibitory nucleic acid is an miRNA, an siRNA, or shRNA.

22. The construct of claim 20 or 21, wherein the KCNQ4 inhibitory RNA comprises a sequence according to SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO:63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 96, or SEQ ID NO: 97.

23. The construct of claim 20, wherein the KCNQ4 inhibitory RNA is a gRNA.

24. The construct of claim 20 or 23, wherein the KCNQ4 inhibitory RNA comprises a sequence according to SEQ ID NO: 42, SEQ ID NO: 43, SEQ ID NO: 44, SEQ ID NO: 45, SEQ ID NO: 46, SEQ ID NO: 47, SEQ ID NO: 48.

25. The construct of claim 20, wherein the KCNQ4 gene is a primate KCNQ4 gene.

26. The construct of claim 20 or 25, wherein the KCNQ4 gene is a human KCNQ4 gene.

27. The construct of claim 26, wherein the human KCNQ4 gene comprises a nucleic acid sequence according to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 25, SEQ ID NO: 26, SEQ ID NO: 27, SEQ ID NO: 28, SEQ ID NO: 29, SEQ ID NO: 30 or SEQ ID NO: 90.

28. The construct of any one of claims 20-27, wherein the promoter is an inducible promoter, a constitutive promoter, or a tissue-specific promoter.

29. The construct of any one of claims 20-28, wherein the promotor is a cochlear hair cell-specific promoter.

30. The construct of claim 29, wherein the cochlear hair cell-specific promoter is a ATOH1 promoter, a POU4F3 promoter, a LHX3 promoter, a MYO7A promoter, a MYO6 promoter, a α9ACHR promoter, or a α10ACHR promoter.

31. The construct of any one of claims 20-28, wherein the promoter is an H1 or U6 promoter.

32. The construct of claim 31, wherein the promoter comprises a nucleic acid sequence according to SEQ ID NO: 22, SEQ ID NO: 23, SEQ ID NO: 24, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308, SEQ ID NO: 309, and/or SEQ ID NO: 310.

33. The construct of any one of claims 20-32, further comprising two AAV inverted terminal repeats (ITRs), wherein the two AAV ITRs flank the coding sequence and promoter.

34. The construct of claim 33, wherein the two AAV ITRs are or are derived from AAV2 ITRs.

35. The construct of claim 33, wherein the two AAV ITRs comprise:
(i) a 5′ ITR comprising a nucleic acid sequence according to SEQ ID NO: 15 and a 3′ ITR comprising a nucleic acid sequence according to SEQ ID NO: 16; or
(ii) a 5′ ITR comprising a nucleic acid sequence according to SEQ ID NO: 19 and a 3′ ITR comprising a nucleic acid sequence according to SEQ ID NO: 20.

36. The construct of claim 1, wherein the construct comprises a nucleic acid sequence according to any of SEQ ID NOs: 1-10.

37. The construct of claim 1, wherein the construct comprises a nucleic acid sequence according to any of SEQ ID NOs: 25-30 or 90-91.

38. An AAV particle comprising the construct of any one of claims 1-19.

39. An AAV particle comprising the construct of any one of claims 20-37.

40. The AAV particle of claim 38 or 39, further comprising an AAV capsid, wherein the AAV capsid is or is derived from an AAV2, AAV3, AAV4, AAV5, AAV6, AAV7, AAV8, AAV9, AAV10, AAV-rh8, AAV-rh10, AAV-rh39, AAV-rh43 or AAV Anc80 capsid.

41. The AAV particle of claim 40, wherein the AAV capsid is an AAV Anc80 capsid.

42. A composition comprising:
(i) the construct of any one of claims 1-19;
(ii) the construct of any one of claims 20-37; or
(iii) a combination thereof.

43. A composition comprising the AAV particle of any one of claims 38-41.

44. A composition comprising:
(i) the AAV particle of claim 38;
(ii) the AAV particle of claim 39; or
(iii) a combination thereof.

45. The composition of claim 44, wherein the AAV particle of (i), (ii), or both further comprise an AAV capsid, wherein the AAV capsid is or is derived from an AAV2, AAV3, AAV4, AAV5, AAV6, AAV7, AAV8, AAV9, AAV10, AAV-rh8, AAV-rh10, AAV-rh39, AAV-rh43 or AAV Anc80 capsid.

46. The composition of claim 44, wherein the AAV capsid of the AAV particle of (i), (ii), or both is an AAV Anc80 capsid.

47. The composition of any one of claims 42-46, wherein the composition is a pharmaceutical composition.

48. The composition of claim 47, further comprising a pharmaceutically acceptable carrier.

49. A cell comprising the composition of any one of claims 42-48.

50. The cell of claim 49, wherein the cell is in vivo, ex vivo, or in vitro.

51. The cell of claim 49 or 50, wherein the cell is a mammalian cell.

52. The cell of claim 51, wherein the mammalian cell is a human cell.

53. The cell of claim 52, wherein the cell is immortalized to generate a stable cell line.

54. The cell of claim 52, wherein the human cell is in the ear of a subject.

55. The cell of claim 52, wherein at least one copy of an endogenous KCNQ4 gene has at least one sequence variation.

56. The cell of claim 55, wherein the at least one sequence variation results in a loss-of-function gene product.

57. A system comprising the composition of any one of claims 42-48.

58. A method comprising contacting an inner ear cell with the composition of any one of claims 42-48.

59. The method of claim 58, where the inner ear cell is an outer hair cell.

60. The method of claim 58 or 59, wherein the inner ear cell is in the ear of a subject.

61. The method of claim 58 or 59, wherein the inner ear cell is in vitro or ex vivo.

62. A method comprising, contacting a cell with:
(i) the construct of any one of claims 1-38; and
(ii) one or more plasmids comprising an AAV Rep gene, AAV Cap gene, AAV VA gene, AAV E2a gene, and AAV E4 gene.

63. The method of claim 62, where the cell is an inner ear cell.

64. The method of claim 63, wherein the inner ear cell is outer hair cell.

65. The method of claim 63, wherein the inner ear cell is in the ear of a subject.

66. The method of claim 58 or 59, wherein the inner ear cell is in vitro or ex vivo.

67. A method comprising introducing the composition of any one of claims 42-48 into the inner ear of a subject.

68. The method of claim 67, wherein the composition is introduced into the cochlea of the subject.

69. The method of claim 67 or 68, wherein the composition is introduced via a round window membrane injection.

70. The method of any one of claim 60, 65, or 67-69, further comprising measuring a hearing level of the subject.

71. The method of claim 70, a hearing level is measured by performing an auditory brainstem response (ABR) test.

72. The method of claim 70 or 71, further comprising comparing the hearing level of the subject to a reference hearing level.

73. The method of claim 72, wherein the reference hearing level is a published or historical reference hearing level.

74. The method of claim 73, wherein the hearing level of the subject is measured after the construct of any one of claims 1-19 is introduced, and the reference hearing level is a hearing level of the subject that was measured before the construct of any one of claims 1-19 was introduced.

75. The method of any one of claims 58-74, further comprising measuring a level of a KCNQ4 gene product in a subject.

76. The method of claim 75, wherein the level of the KCNQ4 gene product is measured in the inner ear of the subject.

77. The method of claim 75, wherein the level of the KCNQ4 gene product is measured in the cochlea of the subject.

78. The method of any one of claims 58-77, further comprising comparing the level of a KCNQ4 gene product in the subject to a reference KCNQ4 gene product level.

79. The method of claim 78, wherein the reference hearing level is a published or historical reference KCNQ4 gene product level.

80. The method of claim 75, wherein the level of a KCNQ4 gene product in the subject is measured after the construct of any one of claims 1-37 is introduced, and the reference KCNQ4 gene product level is a KCNQ4 gene product level of the subject that was measured before the composition of any one of claims 1-37 was introduced.

81. A method of treating hearing loss comprising administering the composition of any one of claims 42-48 to a subject in need thereof.

82. A method of treating hearing loss comprising administering a particle of any one of claims 38-41 to a subject in need thereof.

83. A construct of any one of claims 1-37 for use in the treatment of hearing loss.

84. A composition of any one of claims 42-48 for use in the treatment of hearing loss.

85. A particle of any one of claims 38-41 for use in the treatment of hearing loss.

86. Use of a construct of any one of claims 1-37 for the manufacture of a medicament to treat hearing loss.

87. Use of a composition of any one of claims 42-48 for the manufacture of a medicament to treat hearing loss.

88. Use of a particle of any one of claims 38-41 for the manufacture of a medicament to treat hearing loss.

89. A cell comprising a coding sequence operably linked to a promoter, wherein the coding sequence encodes a Kv7.4 protein.

90. A cell comprising a coding sequence operably linked to a promoter, wherein the coding sequence encodes a loss-of-function KCNQ4 variant gene product.

91. A population of cells comprising one or more cells according to claim 89, wherein the population is or comprises a stable cell line.

92. The method of any one of claims 75-80, wherein the KCNQ4 gene product is a Kv7.4 protein.

93. A construct comprising an inhibitory nucleic acid, wherein the inhibitory nucleic acid is or comprises one or more of miR1-155; miR2-155; miR4-155; miR5-155; miR6-155; miR7-155 miR1-16; miR1-26; miR1-96; miR1-122; miR1-135; miR1-182; miR1-183; miR1-335; miR1-451.

94. An miRNA selected from the group consisting of miR1-155; miR2-155; miR4-155; miR5-155; miR6-155; miR7-155; miR1-16; miR1-26; miR1-96; miR1-122; miR1-135; miR1-182; miR1-183; miR1-335; miR1-451 and combinations thereof.

95. A kit comprising a composition of any one of claims 1-94.

96. The kit of claim 95, wherein the composition is pre-loaded in a device.

97. The kit of claim 96, wherein the device is a microcatheter.

98. The kit of claim 97, wherein the microcatheter is shaped such that it can enter the middle ear cavity via the external auditory canal and contact the end of the microcatheter with the RWM.

99. The kit of claim 97 or 98, wherein a distal end of the microcatheter is comprised of at least one microneedle with diameter of between 10 and 1,000 microns.

100. The kit of any one of claims 95-99, further comprising a device.

101. The kit of claim 100, wherein the device is a device described in FIGS. 32-35 or a device as described herein.

102. The kit of claim 101, wherein the device comprises a needle comprising a bent portion and an angled tip.
